(19) United States
(12) Patent Application Publication
(10) Pub. No.: US 2025/0115598 A1
(43) Pub. Date: Apr. 10, 2025

(54) BIFUNCTIONAL ARYLSULPHONAMIDE COMPOUNDS

(71) Applicant: Anaxis Pharma Pty Ltd, Parkville (AU)

(72) Inventors: Jean-Marc Daniel Garnier, Parkville (AU); Christopher Gardner, Parkville (AU); Guillaume Laurent Lessene, Parkville (AU)

(21) Appl. No.: 18/722,099

(22) PCT Filed: Dec. 22, 2022
(86) PCT No.: PCT/AU22/51576

§ 371 Date: Jun. 20, 2024

(30) Foreign Application Priority Data
Dec. 22, 2021 (AU) 2021904198

(51) Int. Cl. C07D 471/04; A61K 31/416; A61K 31/4355; A61K 31/437; A61K 31/4545; A61K 31/4985; A61K 31/506; A61K 31/519; A61K 31/5377; A61P 43/00; C07D 231/56; C07D 487/04; C07D 491/048; C07D 519/00

(52) U.S. Cl. CPC C07D 471/04; A61K 31/416; A61K 31/4355; A61K 31/437; A61K 31/4545; A61K 31/4985; A61K 31/506; A61K 31/519; A61K 31/5377; A61P 43/00; C07D 231/56; C07D 487/04; C07D 491/048; C07D 519/00

(57) ABSTRACT

This invention relates to compounds of formula (X) and salts, solvates, tautomers, N-oxides, stereoisomers, polymorphs and/or prodrugs thereof. Also disclosed is the use of the compounds of formula (X) to treat necroptosis and/or inhibit and/or degrade MLKL.

### CROSS REFERENCE

This application claims priority to Australian provisional application no. 2021904198, filed on 22 Dec. 2021, the entire contents of which are incorporated herein by reference.

### FIELD

The present disclosure relates to compounds which treat necroptosis and/or inhibit and/or degrade mixed lineage kinase domain-like protein (MLKL), and methods for their use.

### BACKGROUND

In many diseases, cell death is mediated through apoptotic and/or necrotic pathways. While much is known about the mechanisms of action that control apoptosis, control of necrosis is not as well understood. Understanding the mechanisms in respect of both necrosis and apoptosis in cells is essential to being able to treat conditions, such as neurodegenerative diseases, stroke—coronary heart disease, kidney disease, liver disease, AIDS and the conditions associated with AIDS.

Cell death has traditionally been categorized as either apoptotic or necrotic based on morphological characteristics (Wyllie et al., Int. Rev. Cytol. 68: 251 (1980)). These two modes of cell death were also initially thought to occur via regulated (caspase-dependent) and non-regulated processes, respectively. More recent studies, however, demonstrate that the underlying cell death mechanisms resulting in these two phenotypes are much more complicated and under some circumstances interrelated. Furthermore, conditions that lead to necrosis can occur by either regulated caspase-independent or non-regulated processes.

One regulated caspase-independent cell death pathway with morphological features resembling necrosis, called necroptosis, has been described (Degterev et al., Nat. Chem. Biol. 1:112, 2005). This cell death modality can be initiated with various stimuli (e.g., TNF-[alpha] and Fas ligand) and in an array of cell types (e.g., monocytes, fibroblasts, lymphocytes, macrophages, epithelial cells and neurons). Necroptosis may represent a significant contributor to and in some cases predominant mode of cellular demise under pathological conditions involving excessive cell stress, rapid energy loss and massive oxidative species generation, where the highly energy-dependent apoptosis process is not operative.

In WO2015/172203, we reported that particular compounds described in US2005/0085637 have been found to be suitable for inhibiting necroptosis. We also discussed particularly suitable compounds for inhibiting necroptosis in WO2016/127213.

All publications, patents and patent applications that may be cited herein are hereby incorporated by reference in their entirety.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

As discussed above, certain compounds described in WO2016/127213, US2005/0085637 and WO2015/172203 have been found to be suitable for inhibiting necroptosis-associated diseases. Surprisingly, the inventors of this invention have now discovered that other types of compounds are also suitable for inhibiting necroptosis-associated diseases. Further, and equally surprisingly, the inventors of this invention have now discovered that compounds that degrade mixed lineage kinase domain-like protein (MLKL) can also inhibit necroptosis. In addition, the compounds described herein are potent inhibitors and degraders of different MLKL homologues, including human MLKL and murine MLKL. The cross-homologue MLKL potency may provide useful pre-clinical safety and efficacy data.

In one aspect, there is provided a compound according to Formula (X):

MLKLi-L-E3L (X)
wherein:
E3L is an E3 ligase binding moiety
L is a linker covalently linking E3L to MLKLi
MLKLi is a radical of formula (I)

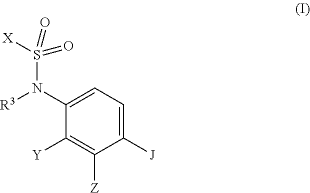

wherein
J is selected from the following moieties J1 and J2:

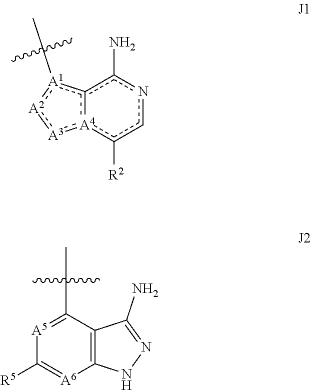

A¹ and A⁴ are independently selected from N and C;
A² and A³ are independently selected from N, NR¹, CH, O and S;
wherein at least one of A¹, A², A³ and A⁴ is selected from N, NR¹, O and S;
A⁵ is CH or N:
A⁶ is N or CR²;
R¹ and R³ are independently selected from H and an optionally substituted C₁₋₆alkyl; R² is selected from:
   (i) H,
   (ii) optionally substituted C₁₋₄alkylamido,
   (iii) optionally substituted C₁₋₄alkylaryl,
   (iv) optionally substituted C₂₋₄alkynyl,
   (v) optionally substituted aryl,
   (vi) optionally substituted 5- or 6-membered heterocyclyl,
   (vii) cyano;
X is selected from optionally substituted C₁₋₆alkyl, optionally substituted haloC₁₋₆alkyl, optionally substituted C₂₋₆alkynyl, optionally substituted cycloalkyl, optionally substituted halocycloalkyl, optionally substituted aryl, optionally substituted alkylaryl, optionally substituted C₁₋₆alkylcycloalkyl and optionally substituted amino;
Y and Z are independently selected from H, R⁴, —OR⁴, —NR⁴R^{4′} and halo;
wherein at least one of Y and Z is H;
R⁴ is selected from optionally substituted C₁₋₆alkyl, optionally substituted aryl, optionally substituted C₁₋₆alkylaryl, optionally substituted heterocyclyl, optionally substituted C₁₋₆alkylheterocyclyl, optionally substituted cycloalkyl, optionally substituted C₁₋₆alkylC₃₋₁₀cycloalkyl, optionally substituted C₃₋₁₀cycloalkylaryl, optionally substituted C₃₋₁₀cycloalkylheterocyclyl, optionally substituted C₃₋₁₀cycloalkylC₃₋₁₀cycloalkyl, optionally substituted 3-6 membered non-aromatic heterocyclyl-aryl, optionally substituted 3-6 membered non-aromatic heterocyclylC₃₋₁₀cycloalkyl and optionally substituted 3-6 membered non-aromatic heterocyclyl-3-10 membered heterocyclyl;
R⁴ is H or optionally substituted C₁₋₆alkyl; and
R⁵ is selected from H, optionally substituted C₁₋₆alkyl, optionally substituted C₃₋₁₀cycloalkyl and optionally substituted aryl.

In some embodiments, R² is selected from:
(i) H,
(ii) optionally substituted C₁₋₄alkylamido,
(iii) optionally substituted C₁₋₄alkylaryl,
(iv) optionally substituted C₂₋₄alkynyl,
(v) optionally substituted aryl,
(vi) optionally substituted 5- or 6-membered heterocyclyl.

In some embodiments, MLKLi is a compound of formula (X) comprising a radical at:
i) R² when J is J1 so that R² and L are covalently bound; or
ii) R⁵ when J is J2 so that R⁵ and L are covalently bound.

In some embodiments, the compound of formula (X) may be provided as a compound of formula (XX):

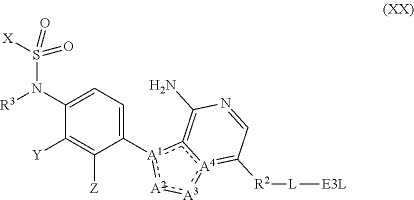

wherein A¹, A², A³, A⁴, n, R¹, R², R³, R⁴, R⁴, X, Y, Z, L and E3L are as defined herein.

In some embodiments, the compound of formula (X) may be provided as a compound of formula (XXa):

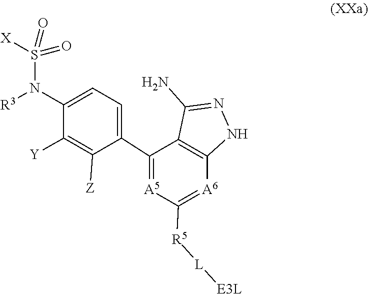

wherein A⁵, A⁶, R¹, R², R³, R⁴, R⁴, R⁵, X, Y, Z, L and E3L are as defined herein.

In any aspect or embodiment described herein, the compound of the invention may be provided in the form of a pharmaceutically acceptable salt, solvate, tautomer, N-oxide, stereoisomer and/or prodrug thereof.

The inventors have found that compounds of Formula (I) are selective degraders of MLKL. Degradation of MLKL may be preferred to inhibition of MLKL in some instances as degradation results in a loss of function of the degraded protein, while the effects of inhibition last only as long as the inhibitor interacts with the protein.

In some embodiments, the compound of the invention is selected from any of compounds 1001 to 1031, 1037, and 1039 to 1145 described herein, preferably from any of compounds 1001 to 1031, 1037, and 1039 to 1137.

In some embodiments, the compound of the invention is selected from any of compounds 1001 to 1031, preferably from any of compounds 1001 to 1023 and 1029 to 1031, more preferably from any of compounds 1003, 1005, 1006, 1008 to 1012, 1014, 1015, 1017 and 1022.

In some embodiments, the compound of the invention comprises a radical of any of compounds 1-173 described herein.

In some embodiments, the compound of the invention comprises a radical of any of compounds 7, 9, 12, 13, 14, 15, 16, 18, 19, 20 and 72.

In some embodiments, the compound of the invention comprises a radical of any of compounds 1, 7, 9, 12-16, 18-21, 23-32, 35-46, 48, 50-52, 55-62, 72-89, 92-109, 111, 113, 115-117, and 119-123, preferably a radical of any of compounds 19, 29-31, 36, 38, 40-41. 75-77, 81, 100-101, 103-104, 109, 120 and 123.

In some embodiments, the compound of the invention comprises a radical of any of compounds 1, 5, 7-9, 11-16, 18-21, 23-32, 35-46, 48, 50-52, 55-62, 66, 69, 70, 72-89, 92-109, 111, 113, 115-117, and 119-123, preferably a radical of any of compounds 5, 7-9, 11-13, 15, 18-20, 60, 61, 66, 69, 70 and 72.

In some embodiments, the compound of the invention comprises a radical of any of compounds 3, 11, 13, 19, 29, 30, 46, 79, 80, 81, 97, 98, 99, 103, 104, 109, 113, 116, 120, 121 and 122.

In another aspect, there is provided a medicament comprising a compound of the invention, and the use of the compound of the invention in the preparation of a medicament. That medicament may be for treating necroptosis; the medicament may also be for degrading MLKL.

In another aspect, there is provided a pharmaceutical composition comprising a compound of the invention and optionally a pharmaceutically acceptable excipient.

In another aspect, there is provided a method of treating necroptosis, comprising administering to a subject in need thereof an effective amount of a compound of the invention.

In another aspect, there is provided a method of degrading MLKL, comprising contacting a cell with a compound of the invention.

In another aspect, there is provided a compound of the invention for use in treating necroptosis, and for use in degrading MLKL.

Any embodiment herein shall be taken to apply mutatis mutandis to any other embodiment unless specifically stated otherwise.

The present disclosure is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### Definitions

Unless otherwise herein defined, the following terms will be understood to have the general meanings which follow.

The term “C₁₋₆alkyl” refers to optionally substituted straight chain or branched chain hydrocarbon groups having from 1 to 6 carbon atoms. Examples include methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, hexyl and the like. Unless the context requires otherwise, the term “C₁₋₆alkyl” also encompasses alkyl groups containing one less hydrogen atom such that the group is attached via two positions i.e. divalent. “C₁₋₄alkyl” and “C₁₋₃alkyl” including methyl, ethyl, propyl, isopropyl, n-butyl, iso-butyl, sec-butyl and tert-butyl are preferred with methyl being particularly preferred.

The term “C₂₋₆alkenyl” refers to optionally substituted straight chain or branched chain hydrocarbon groups having at least one double bond of either E or Z stereochemistry where applicable and 2 to 6 carbon atoms. Examples include vinyl, 1-propenyl, 1- and 2-butenyl and 2-methyl-2-propenyl. Unless the context requires otherwise, the term “C₂₋₆alkenyl” also encompasses alkenyl groups containing one less hydrogen atom such that the group is attached via two positions i.e. divalent. “C₂₋₄alkenyl” and “C₂₋₃alkenyl” including ethenyl, propenyl and butenyl are preferred with ethenyl being particularly preferred.

The term “C₂₋₆alkynyl” refers to optionally substituted straight chain or branched chain hydrocarbon groups having at least one triple bond and 2 to 6 carbon atoms. Examples include ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl and the like. Unless the context indicates otherwise, the term “C₂₋₆alkynyl” also encompasses alkynyl groups containing one less hydrogen atom such that the group is attached via two positions i.e. divalent. C₂₋₄alkynyl or C₂₋₃alkynyl are preferred.

The term “C₃₋₁₀cycloalkyl” refers to non-aromatic cyclic groups having from 3 to 10 carbon atoms, including cyclopropyl, cyclobutyl, cyclopentyl, cyclohexyl, cycloheptyl, cyclooctyl, cyclononyl and cyclodecyl. It will be understood that cycloalkyl groups may be saturated such as cyclohexyl or unsaturated such as cyclohexenyl. C₃₋₆cycloalkyl such as cyclopropyl, cyclobutyl, cyclopentyl and cyclohexyl are preferred. Cycloalkyl groups also include polycyclic carbocycles and include fused, bridged and spirocyclic systems.

The terms “hydroxy” and “hydroxyl” refer to the group —OH.

The term “oxo” refers to the group ═O.

The term “C₁₋₆alkoxy” refers to an alkyl group as defined above covalently bound via an O linkage containing 1 to 6 carbon atoms, such as methoxy, ethoxy, propoxy, isoproxy, butoxy, tert-butoxy and pentoxy. “C₁₋₄alkoxy” and “C₁₋₃alkoxy” including methoxy, ethoxy, propoxy and butoxy are preferred with methoxy being particularly preferred.

The terms “haloC₁₋₆alkyl” and “C₁₋₆alkylhalo” refer to a C₁₋₆alkyl which is substituted with one or more halogens. HaloC₁₋₃alkyl groups are preferred, such as for example, —CH₂CF₃, and —CF₃.

The terms “haloC₁₋₆alkoxy” and “C₁₋₆alkoxyhalo” refer to a C₁₋₆alkoxy which is substituted with one or more halogens. C₁₋₃alkoxyhalo groups are preferred, such as for example, —OCF₃.

The term “carboxylate” or “carboxyl” refers to the group —COO— or —COOH.

The term “ester” refers to a carboxyl group having the hydrogen replaced with, for example a C₁₋₆alkyl group (“carboxylC₁₋₆alkyl” or “alkylester”), an aryl or aralkyl group (“arylester” or “aralkylester”) and so on. CO₂C₁₋₃alkyl groups are preferred, such as for example, methylester (CO₂Me), ethylester (CO₂Et) and propylester (CO₂Pr) and includes reverse esters thereof (e.g. —OC(O)Me, —OC(O)Et and —OC(O)Pr).

The terms “cyano” and “nitrile” refer to the group —CN.

The term “nitro” refers to the group —NO₂.

The term “amino” refers to the group —NH₂.

The term “substituted amino” refers to an amino group having at least one hydrogen replaced with, for example a C₁₋₆alkyl group (“C₁₋₆alkylamino”), an aryl or aralkyl group (“arylamino”, “aralkylamino”) and so on. Substituted amino groups include “monosubstituted amino” (or “secondary amino”) groups, which refer to an amino group having a single hydrogen replaced with, for example a C₁₋₆alkyl group, an aryl or aralkyl group and so on. Preferred secondary amino groups include C₁₋₃alkylamino groups, such as for example, methylamino (NHMe), ethylamino (NHEt) and propylamino (NHPr). Substituted amino groups also include “disubstituted amino” (or “tertiary amino”) groups, which refer to amino groups having both hydrogens replaced with, for example C₁₋₆alkyl groups, which may be the same or different (“dialkylamino”), aryl and alkyl groups (“aryl(alkyl)amino”) and so on. Preferred tertiary amino groups include di(C₁₋₃alkyl)amino groups, such as for example, dimethylamino (NMe₂), diethylamino (NEt₂), dipropylamino (NPr₂) and variations thereof (e.g. N(Me)(Et) and so on).

The term “aldehyde” refers to the group —C(═O)H.

The terms “acyl” and “acetyl” refers to the group —C(O)CH₃.

The term “ketone” refers to a carbonyl group which may be represented by —C(O)—.

The term “substituted ketone” refers to a ketone group covalently linked to at least one further group, for example, a C₁₋₆alkyl group (“C₁₋₆alkylacyl” or “alkylketone” or “ketoalkyl”), an aryl group (“arylketone”), an aralkyl group (“aralkylketone) and so on. C₁₋₃alkylacyl groups are preferred.

The term “amido” or “amide” refers to the group —C(O)NH₂.

The term “substituted amido” or “substituted amide” refers to an amido group having a hydrogen replaced with, for example a C₁₋₆alkyl group (“C₁₋₆alkylamido” or “C₁₋₆alkylamide”), an aryl (“arylamido”), aralkyl group (“aralkylamido”) and so on. C₁₋₃alkylamide groups are preferred, such as for example, methylamide (—C(O)NHMe), ethylamide (—C(O)NHEt) and propylamide (—C(O)NHPr) and includes reverse amides thereof (e.g. —NHMeC(O)—, —NHEtC(O)— and —NHPrC(O)—).

The term “disubstituted amido” or “disubstituted amide” refers to an amido group having the two hydrogens replaced with, for example a C₁₋₆alkyl group (“di(C₁₋₆alkyl)amido” or “di(C₁₋₆ alkyl)amide”), an aralkyl and alkyl group (“alkyl(aralkyl)amido”) and so on. Di(C₁₋₃alkyl)amide groups are preferred, such as for example, dimethylamide (—C(O)NMe₂), diethylamide (—C(O)NEt₂) and dipropylamide ((—C(O)NPr₂) and variations thereof (e.g. —C(O)N(Me)Et and so on) and includes reverse amides thereof.

The term “thiol” refers to the group —SH.

The term “C₁₋₆alkylthio” refers to a thiol group having the hydrogen replaced with a C₁₋₆alkyl group. C₁₋₃alkylthio groups are preferred, such as for example, thiolmethyl, thiolethyl and thiolpropyl.

The terms “thioxo” refer to the group ═S.

The term “sulfinyl” refers to the group —S(═O)H.

The term “substituted sulfinyl” or “sulfoxide” refers to a sulfinyl group having the hydrogen replaced with, for example a C₁₋₆alkyl group (“C₁₋₆alkylsulfinyl” or “C₁₋₆alkylsulfoxide”), an aryl (“arylsulfinyl”), an aralkyl (“aralkyl sulfinyl”) and so on. C₁₋₃alkylsulfinyl groups are preferred, such as for example, —SOmethyl, —SOethyl and —SOpropyl.

The term “sulfonyl” refers to the group —SO₂H.

The term “substituted sulfonyl” refers to a sulfonyl group having the hydrogen replaced with, for example a C₁₋₆alkyl group (“sulfonylC₁₋₆alkyl”), an aryl (“arylsulfonyl”), an aralkyl (“aralkylsulfonyl”) and so on. SulfonylC₁₋₃alkyl groups are preferred, such as for example, —SO₂Me, —SO₂Et and —SO₂Pr.

The term “sulfonylamido” or “sulfonamide” refers to the group —SO₂NH₂.

The term “substituted sulfonamido” or “substituted sulphonamide” refers to an sulfonylamido group having a hydrogen replaced with, for example a C₁₋₆alkyl group (“sulfonylamidoC₁₋₆ alkyl”), an aryl (“arylsulfonamide”), aralkyl (“aralkylsulfonamide”) and so on. SulfonylamidoC₁₋₃alkyl groups are preferred, such as for example, —SO₂NHMe, —SO₂NHEt and —SO₂NHPr and includes reverse sulfonamides thereof (e.g. —NHSO₂Me, —NHSO₂Et and —NHSO₂Pr).

The term “disubstituted sufonamido” or “disubstituted sulphonamide” refers to an sulfonylamido group having the two hydrogens replaced with, for example a C₁₋₆alkyl group, which may be the same or different (“sulfonylamidodi(C₁₋₆alkyl)”), an aralkyl and alkyl group (“sulfonamido(aralkyl)alkyl”) and so on. Sulfonylamidodi(C₁₋₃alkyl) groups are preferred, such as for example, —SO₂NMe₂, —SO₂NEt₂ and —SO₂NPr₂ and variations thereof (e.g. —SO₂N(Me)Et and so on) and includes reserve sulfonamides thereof (e.g. —N(Me)SO₂Me and so on).

The term “sulfate” refers to the group OS(O)₂OH and includes groups having the hydrogen replaced with, for example a C₁₋₆alkyl group (“alkylsulfates”), an aryl (“arylsulfate”), an aralkyl (“aralkylsulfate”) and so on. C₁₋₃sulfates are preferred, such as for example, OS(O)₂OMe, OS(O)₂OEt and OS(O)₂OPr.

The term “sulfonate” refers to the group SO₃H and includes groups having the hydrogen replaced with, for example a C₁₋₆alkyl group (“alkylsulfonate”), an aryl (“arylsulfonate”), an aralkyl (“aralkylsulfonate”) and so on. C₁₋₃sulfonates are preferred, such as for example, SO₃Me, SO₃Et and SO₃Pr.

The term “aryl” refers to a carbocyclic (non-heterocyclic) aromatic ring or mono-, bi- or tri-cyclic ring system. Poly-cyclic ring systems may be referred to as “aryl” provided at least 1 of the rings within the system is aromatic. The aromatic ring or ring system is generally composed of 6 to 10 carbon atoms. Examples of aryl groups include but are not limited to phenyl, biphenyl, naphthyl and tetrahydronaphthyl. 6-membered aryls such as phenyl are preferred. The term “alkylaryl” refers to C₁₋₆alkylaryl such as benzyl.

The term “alkoxyaryl” refers to C₁₋₆alkyloxyaryl such as benzyloxy.

The term “heterocyclyl” refers to a moiety obtained by removing a hydrogen atom from a ring atom of a heterocyclic compound which moiety has from 3 to 10 ring atoms (unless otherwise specified), of which 1, 2, 3 or 4 are ring heteroatoms each heteroatom being independently selected from O, S and N. Heterocyclyl groups include monocyclic and polycyclic (such as bicyclic) ring systems, such as fused, bridged and spirocyclic systems, provided at least one of the rings of the ring systm contains at least one heteroatom.

In this context, the prefixs 3-, 4-, 5-, 6-, 7-, 8-, 9- and 10-membered denote the number of ring atoms, or range of ring atoms, whether carbon atoms or heteroatoms. For example, the term “3-10 membered heterocylyl”, as used herein, pertains to a heterocyclyl group having 3, 4, 5, 6, 7, 8, 9 or 10 ring atoms. Examples of heterocylyl groups include 5-6-membered monocyclic heterocyclyls and 9-10 membered fused bicyclic heterocyclyls.

Examples of monocyclic heterocyclyl groups include, but are not limited to, those containing one nitrogen atom such as aziridine (3-membered ring), azetidine (4-membered ring), pyrrolidine (tetrahydropyrrole), pyrroline (e.g., 3-pyrroline, 2,5-dihydropyrrole), 2H-pyrrole or 3H-pyrrole (isopyrrole, isoazole) or pyrrolidinone (5-membered rings), piperidine, dihydropyridine, tetrahydropyridine (6-membered rings), and azepine (7-membered ring); those containing two nitrogen atoms such as imidazoline, pyrazolidine (diazolidine), imidazoline, pyrazoline (dihydropyrazole) (5-membered rings), piperazine (6-membered ring); those containing one oxygen atom such as oxirane (3-membered ring), oxetane (4-membered ring), oxolane (tetrahydrofuran), oxole (dihydrofuran) (5-membered rings), oxane (tetrahydropyran), dihydropyran, pyran (6-membered rings), oxepin (7-membered ring); those containing two oxygen atoms such as dioxolane (5-membered ring), dioxane (6-membered ring), and dioxepane (7-membered ring); those containing three oxygen atoms such as trioxane (6-membered ring); those containing one sulfur atom such as thiirane (3-membered ring), thietane (4-membered ring), thiolane (tetrahydrothiophene) (5-membered ring), thiane (tetrahydrothiopyran) (6-membered ring), thiepane (7-membered ring); those containing one nitrogen and one oxygen atom such as tetrahydrooxazole, dihydrooxazole, tetrahydroisoxazole, dihydroisoxazole (5-membered rings), morpholine, tetrahydrooxazine, dihydrooxazine, oxazine (6-membered rings); those containing one nitrogen and one sulfur atom such as thiazoline, thiazolidine (5-membered rings), thiomorpholine (6-membered ring); those containing two nitrogen and one oxygen atom such as oxadiazine (6-membered ring); those containing one oxygen and one sulfur such as: oxathiole (5-membered ring) and oxathiane (thioxane) (6-membered ring); and those containing one nitrogen, one oxygen and one sulfur atom such as oxathiazine (6-membered ring).

Heterocyclyls encompass aromatic heterocyclyls and non-aromatic heterocyclyls. Such groups may be substituted or unsubstituted.

The term “aromatic heterocyclyl” may be used interchangeably with the term “heteroaromatic” or the term “heteroaryl” or “hetaryl”. The heteroatoms in the aromatic heterocyclyl group may be independently selected from N, S and O. The aromatic heterocyclyl groups may comprise 1, 2, 3, 4 or more ring heteroatoms. In the case of fused aromatic heterocyclyl groups, only one of the rings may contain a heteroatom and not all rings must be aromatic.

“Heteroaryl” is used herein to denote a heterocyclic group having aromatic character and embraces aromatic monocyclic ring systems and polycyclic (e.g. bicyclic) ring systems containing one or more aromatic rings. The term aromatic heterocyclyl also encompasses pseudoaromatic heterocyclyls. The term “pseudoaromatic” refers to a ring system which is not strictly aromatic, but which is stabilized by means of delocalization of electrons and behaves in a similar manner to aromatic rings. The term aromatic heterocyclyl therefore covers polycyclic ring systems in which all of the fused rings are aromatic as well as ring systems where one or more rings are non-aromatic, provided that at least one ring is aromatic. In polycyclic systems containing both aromatic and non-aromatic rings fused together, the group may be attached to another moiety by the aromatic ring or by a non-aromatic ring.

Examples of heteroaryl groups are monocyclic and bicyclic groups containing from five to ten ring members. The heteroaryl group can be, for example, a five membered or six membered monocyclic ring or a bicyclic structure formed from fused five and six membered rings or two fused six membered rings or two fused five membered rings. Each ring may contain up to about four heteroatoms typically selected from nitrogen, sulphur and oxygen. The heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Aromatic heterocyclyl groups may be 5-membered or 6-membered mono-cyclic aromatic ring systems.

Examples of 5-membered monocyclic heteroaryl groups include but are not limited to furanyl, thienyl, pyrrolyl, oxazolyl, oxadiazolyl (including 1,2,3 and 1,2,4 oxadiazolyls and furazanyl i.e. 1,2,5-oxadiazolyl), thiazolyl, isoxazolyl, isothiazolyl, pyrazolyl, imidazolyl, triazolyl (including 1,2,3, 1,2,4 and 1,3,4 triazolyls), oxatriazolyl, tetrazolyl, thiadiazolyl (including 1,2,3 and 1,3,4 thiadiazolyls) and the like.

Examples of 6-membered monocyclic heteroaryl groups include but are not limited to pyridinyl, pyrimidinyl, pyridazinyl, pyrazinyl, triazinyl, pyranyl, oxazinyl, dioxinyl, thiazinyl, thiadiazinyl and the like. Examples of 6-membered aromatic heterocyclyls containing nitrogen include pyridyl (1 nitrogen), pyrazinyl, pyrimidinyl and pyridazinyl (2 nitrogens).

Aromatic heterocyclyl groups may also be bicyclic or polycyclic heteroaromatic ring systems such as fused ring systems (including purine, pteridinyl, napthyridinyl, 1H thieno[2,3-c]pyrazolyl, thieno[2,3-b]furyl and the like) or linked ring systems (such as oligothiophene, polypyrrole and the like). Fused ring systems may also include aromatic 5-membered or 6-membered heterocyclyls fused to carbocyclic aromatic rings such as phenyl, naphtyl, indenyl, azulenyl, fluorenyl, anthracenyl and the like, such as 5-membered aromatic heterocyclyls containing nitrogen fused to phenyl rings, 5-membered aromatic heterocyclyls containing 1 or 2 nitrogens fused to phenyl ring.

A bicyclic heteroaryl group may be, for example, a group selected from: a) a benzene ring fused to a 5- or 6-membered ring containing 1, 2 or 3 ring heteroatoms; b) a pyridine ring fused to a 5- or 6-membered ring containing 1, 2 or 3 ring heteroatoms; c) a pyrimidine ring fused to a 5- or 6-membered ring containing 1 or 2 ring heteroatoms; d) a pyrrole ring fused to a 5- or 6-membered ring containing 1, 2 or 3 ring heteroatoms; e) a pyrazole ring fused to a 5- or 6-membered ring containing 1 or 2 ring heteroatoms; f an imidazole ring fused to a 5- or 6-membered ring containing 1 or 2 ring heteroatoms; g) an oxazole ring fused to a 5- or 6-membered ring containing 1 or 2 ring heteroatoms; h) an isoxazole ring fused to a 5- or 6-membered ring containing 1 or 2 ring heteroatoms; i) a thiazole ring fused to a 5- or 6-membered ring containing 1 or 2 ring heteroatoms; j) an isothiazole ring fused to a 5- or 6-membered ring containing 1 or 2 ring heteroatoms; k) a thiophene ring fused to a 5- or 6-membered ring containing 1, 2 or 3 ring heteroatoms; l) a furan ring fused to a 5- or 6-membered ring containing 1, 2 or 3 ring heteroatoms; m) a cyclohexyl ring fused to a 5- or 6-membered ring containing 1, 2 or 3 ring heteroatoms; and n) a cyclopentyl ring fused to a 5- or 6-membered ring containing 1, 2 or 3 ring heteroatoms.

Particular examples of bicyclic heteroaryl groups containing a five membered ring fused to another five membered ring include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring include but are not limited to benzofuran, benzothiophene, benzimidazole, benzoxazole, isobenzoxazole, benzisoxazole, benzothiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), benzodioxole and pyrazolopyridine (e.g. pyrazolo[1,5-a]pyridine) groups. A further example of a six membered ring fused to a five membered ring is a pyrrolopyridine group such as a pyrrolo[2,3-b]pyridine group.

Particular examples of bicyclic heteroaryl groups containing two fused six membered rings include but are not limited to quinoline, isoquinoline, chroman, thiochroman, chromene, isochromene, isochroman, benzodioxan, quinolizine, benzoxazine, benzodiazine, pyridopyridine, quinoxaline, quinazoline, cinnoline, phthalazine, naphthyridine and pteridine groups.

Examples of heteroaryl groups containing an aromatic ring and a non-aromatic ring include tetrahydronaphthalene, tetrahydroisoquinoline, tetrahydroquinoline, dihydrobenzothiophene, dihydrobenzofuran, 2,3-dihydro-benzo[1,4]dioxine, benzo[1,3]dioxole, 4,5,6,7-tetrahydrobenzofuran, indoiine, isoindoline and indane groups.

Examples of aromatic heterocyclyls fused to carbocyclic aromatic rings may therefore include but are not limited to benzothiophenyl, indolyl, isoindolyl, benzofuranyl, isobenzofuranyl, benzimidazolyl, indazolyl, benzoxazolyl, benzisoxazolyl, isobenzoxazoyl, benzothiazolyl, benzisothiazolyl, quinolinyl, isoquinolinyl, quinoxalinyl, quinazolinyl, cinnolinyl, benzotriazinyl, phthalazinyl, carbolinyl and the like.

The term “non-aromatic heterocyclyl” encompasses optionally substituted saturated and unsaturated rings which contain at least one heteroatom selected from the group consisting of N, S and O. The ring may contain 1, 2 or 3 heteroatoms. The ring may be a monocyclic ring or part of a polycyclic ring system. Polycyclic ring systems include fused rings and spirocycles. Not every ring in a non-aromatic heterocyclic polycyclic ring system must contain a heteroatom, provided at least one ring contains one or more heteroatoms.

Non-aromatic heterocyclyls may be 3-7 membered mono-cyclic rings.

Examples of 5-membered non-aromatic heterocyclyl rings include 2H-pyrrolyl, 1-pyrrolinyl, 2-pyrrolinyl, 3-pyrrolinyl, pyrrolidinyl, 1-pyrrolidinyl, 2-pyrrolidinyl, 3-pyrrolidinyl, tetrahydrofuranyl, tetrahydrothiophenyl, pyrazolinyl, 2-pyrazolinyl, 3-pyrazolinyl, pyrazolidinyl, 2-pyrazolidinyl, 3-pyrazolidinyl, imidazolidinyl, 3-dioxalanyl, thiazolidinyl, isoxazolidinyl, 2-imidazolinyl and the like.

Examples of 6-membered non-aromatic heterocyclyls include piperidinyl, piperidinonyl, pyranyl, dihyrdopyranyl, tetrahydropyranyl, 2H pyranyl, 4H pyranyl, thianyl, thianyl oxide, thianyl dioxide, piperazinyl, diozanyl, 1,4-dioxinyl, 1,4-dithianyl, 1,3,5-triozalanyl, 1,3,5-trithianyl, 1,4-morpholinyl, thiomorpholinyl, 1,4-oxathianyl, triazinyl, 1,4-thiazinyl and the like.

Examples of 7-membered non-aromatic heterocyclyls include azepanyl, oxepanyl, thiepanyl and the like.

Non-aromatic heterocyclyl rings may also be bicyclic heterocyclyl rings such as linked ring systems (for example uridinyl and the like) or fused ring systems. Fused ring systems include non-aromatic 5-membered, 6-membered or 7-membered heterocyclyls fused to carbocyclic aromatic rings such as phenyl, napthyl, indenyl, azulenyl, fluorenyl, anthracenyl and the like. Examples of non-aromatic 5-membered, 6-membered or 7-membered heterocyclyls fused to carbocyclic aromatic rings include indolinyl, benzodiazepinyl, benzazepinyl, dihydrobenzofuranyl and the like.

The term “halo” refers to fluoro, chloro, bromo or iodo.

Unless otherwise defined, the term “optionally substituted” or “optional substituent” as used herein refers to a group which may or may not be further substituted with 1, 2, 3, 4 or more groups, preferably 1, 2 or 3, more preferably 1 or 2 groups selected from the group consisting of C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆alkoxyaryl, halo, C₁₋₆alkylhalo (such as CF₃), C₁₋₆alkoxyhalo (such as OCF₃), carboxyl, esters, cyano, nitro, amino, substituted amino, disubstituted amino, acyl, ketones, substituted ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arC₁₋₆alkyl, heterocyclyl and heteroaryl wherein each alkyl, alkenyl, alkynyl, cycloalkyl, aryl and heterocyclyl and groups containing them may be further optionally substituted. Optional substituents in the case of heterocycles containing N may also include but are not limited to C₁₋₆alkyl i.e. N—C₁₋₃alkyl, more preferably methyl particularly N-methyl.

For optionally substituted “C₁₋₆alkyl”, “C₂₋₆alkenyl” and “C₂₋₆alkynyl”, the optional substituent or substituents are preferably selected from halo, aryl, heterocyclyl, C₃₋₈cycloalkyl, C₁₋₆ alkoxy, hydroxyl, oxo, aryloxy, haloC₁₋₆alkyl, haloC₁₋₆alkoxyl and carboxyl. Each of these optional substituents may also be optionally substituted with any of the optional substituents referred to above, where nitro, amino, substituted amino, cyano, heterocyclyl (including non-aromatic heterocyclyl and heteroaryl), C₁₋₆alkyl, C₂₋₆akenyl, C₂₋₆alkynyl, C₁₋₆alkoxyl, haloC₁₋₆ alkyl, haloC₁₋₆alkoxy, halo, hydroxyl and carboxyl are preferred.

It will be understood that suitable derivatives of aromatic heterocyclyls containing nitrogen include N-oxides thereof.

In the case of hybrid naming of substituent radicals describing two moieties that may both form a bond attaching the radical to the rest of the compound, such as alkylamino and alkylaryl, no direction in the order of groups is intended, so the point of attachment may be to any of the moieties included in the hybrid radical. For example, the terms “alkylaryl” and “arylalkyl”, are intended to refer to the same group and the point of attachment may be via the alkyl or the aryl moiety (or both in the case of diradical species). The direction of attachment of such a hybrid radical may be denoted by inclusion of a bond, for example, “-alkylaryl” or “arylalkyl-” denotes that the point of attachment of the radical to the rest of the compound is via the alkyl moiety, and “alkylaryl-” or “-arylalkyl” denotes that the point of attachment is via the aryl moiety.

It will be appreciated that [symbol] denotes a single or a double bond, depending on the required valency. Unless specifically stated otherwise, where all bonds in a ring are denoted by [symbol] the ring is intended to be aromatic.

As used herein, except where the context requires otherwise, the term “comprise” and variations of the term, such as “comprising”, “comprises” and “comprised”, are not intended to exclude further additives, components, integers or steps.

It must be noted that as used herein and in the appended claims, the singular forms “a”, “an” and “the” include plural reference unless the context clearly dictates otherwise. Thus, for example, a reference to “a salt” may include a plurality of salts and a reference to “at least one heteroatom” may include one or more heteroatoms, and so forth.

The term “and/or” can mean “and” or “or”.

The term “(s)” following a noun contemplates the singular or plural form, or both.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within ±10%, 5%, 1% or ±0.1% of that value.

Further aspects of the present invention and further embodiments of the aspects described in the preceding paragraphs will become apparent from the following description, given by way of example and with reference to the accompanying drawings.

### DETAILED DESCRIPTION OF EMBODIMENTS

The invention provides a compound of (X):

MLKLi-L-E3L (X)
wherein:
E3L is an E3 ligase binding moiety
L is a linker covalently linking E3L to MLKLi
MLKLi is a radical of formula (I)

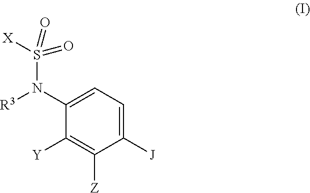

wherein
J is selected from the following moieties J1 and J2:

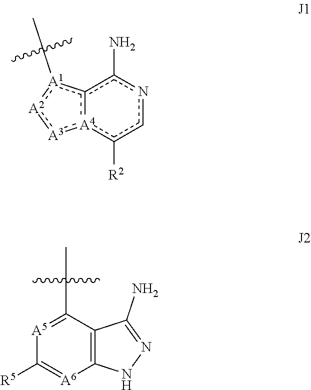

A¹ and A⁴ are independently selected from N and C;
A² and A³ are independently selected from N, NR¹, CH, O and S;
wherein at least one of A¹, A², A³ and A⁴ is selected from N, NR¹, O and S;
A⁵ is CH or N;
A⁶ is N or CR²;
R¹ and R³ are independently selected from H and an optionally substituted C₁₋₆alkyl;
R² is selected from:
   (i) H,
   (ii) optionally substituted C₁₋₄alkylamido,
   (iii) optionally substituted C₁₋₄alkylaryl,
   (iv) optionally substituted C₂₋₄alkynyl,
   (v) optionally substituted aryl,
   (vi) optionally substituted 5- or 6-membered heterocyclyl,
   (vii) cyano;
X is selected from optionally substituted C₁₋₆alkyl, optionally substituted haloC₁₋₆alkyl, optionally substituted C₂₋₆alkynyl, optionally substituted cycloalkyl, optionally substituted halocycloalkyl, optionally substituted aryl, optionally substituted alkylaryl, optionally substituted C₁₋₆alkylcycloalkyl and optionally substituted amino;
Y and Z are independently selected from H, R⁴, —OR⁴, —NR⁴R⁴ and halo;
wherein at least one of Y and Z is H;
R⁴ is selected from optionally substituted C₁₋₆alkyl, optionally substituted aryl, optionally substituted C₁₋₆alkylaryl, optionally substituted heterocyclyl, optionally substituted C₁₋₆alkylheterocyclyl, optionally substituted cycloalkyl, optionally substituted C₁₋₆alkylC₃₋₁₀cycloalkyl, optionally substituted C₃₋₁₀cycloalkylaryl, optionally substituted C₃₋₁₀cycloalkylheterocyclyl, optionally substituted C₃₋₁₀cycloalkylC₃₋₁₀cycloalkyl, optionally substituted 3-6 membered non-aromatic heterocyclyl-aryl, optionally substituted 3-6 membered non-aromatic heterocyclylC₃₋₁₀cycloalkyl and optionally substituted 3-6 membered non-aromatic heterocyclyl-3-10 membered heterocyclyl;
R⁴ is H or optionally substituted C₁₋₆alkyl; and
R⁵ is selected from H, optionally substituted C₁₋₆alkyl, optionally substituted C₃₋₁₀cycloalkyl and optionally substituted aryl.

### MLKLi

In the compounds of the invention, the MLKLi is a radical of a compound of formula (I). Various embodiments of the compound of formula (I) are described below. It will be appreciated that in the compound of formula (X) of the invention, any of the compounds described herein capable of MLKL binding may be included as the MLKLi moiety.

In some embodiments, there is provided a compound of formula (I)

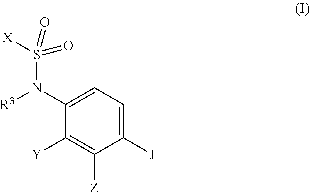

wherein
J is selected from the following moieties J1 and J2:

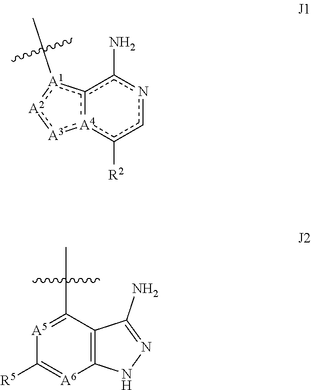

A¹ and A⁴ are independently selected from N and C;
A² and A³ are independently selected from N, NR¹, CH, O and S;
wherein at least one of A¹, A², A³ and A⁴ is selected from N, NR¹, O and S;
A⁵ is CH or N;
A⁶ is N or CR²;
R¹ and R³ are independently selected from H and an optionally substituted C₁₋₆alkyl;
R² is selected from:
   (i) H,
   (ii) optionally substituted C₁₋₄alkylamido,
   (iii) optionally substituted C₁₋₄alkylaryl,
   (iv) optionally substituted C₂₋₄alkynyl,
   (v) optionally substituted aryl,
   (vi) optionally substituted 5- or 6-membered heterocyclyl,
   (vii) cyano;
X is selected from optionally substituted C₁₋₆alkyl, optionally substituted haloC₁₋₆alkyl, optionally substituted C₂₋₆alkynyl, optionally substituted cycloalkyl, optionally substituted halocycloalkyl, optionally substituted aryl, optionally substituted alkylaryl, optionally substituted C₁₋₆alkylcycloalkyl and optionally substituted amino;
Y and Z are independently selected from H, R⁴, —OR⁴, —NR⁴R⁴ and halo;
wherein at least one of Y and Z is H;
R⁴ is selected from optionally substituted C₁₋₆alkyl, optionally substituted aryl, optionally substituted C₁₋₆alkylaryl, optionally substituted heterocyclyl, optionally substituted C₁₋₆alkylheterocyclyl, optionally substituted cycloalkyl, optionally substituted C₁₋₆alkylC₃₋₁₀cycloalkyl, optionally substituted C₃₋₁₀cycloalkylaryl, optionally substituted C₃₋₁₀cycloalkylheterocyclyl, optionally substituted C₃₋₁₀cycloalkylC₃₋₁₀cycloalkyl, optionally substituted 3-6 membered non-aromatic heterocyclyl-aryl, optionally substituted 3-6 membered non-aromatic heterocyclylC₃₋₁₀cycloalkyl and optionally substituted 3-6 membered non-aromatic heterocyclyl-3-10 membered heterocyclyl;
R⁴ is H or optionally substituted C₁₋₆alkyl; and
R⁵ is selected from H, optionally substituted C₁₋₆alkyl, optionally substituted C₃₋₁₀cycloalkyl and optionally substituted aryl.

### X

In some embodiments, X is selected from C₁₋₆alkyl, C₂₋₆alkynyl, C₃₋₆cycloalkyl, aryl, —(CH₂)ₙaryl, —(CH₂)ₙcycloalkyl, and —N(C₁₋₄alkyl)₂;
wherein
n is 1 or 2, and
each alkyl and alkynyl is optionally substituted with one or more groups selected from halo, nitrile, —OR⁶, —N(R⁷)R⁸;
R⁶, R⁷ and R⁸ are independently selected from H, C₁₋₆alkyl and haloC₁₋₆alkyl, and
wherein each aryl and cycloalkyl is optionally substituted with one or more groups that are independently selected from halo, nitrile, C₁₋₄alkyl, C₁₋₄alkoxy, haloC₁₋₄alkyl and haloC₁₋₄ alkoxy.

In some embodiments, X is selected from optionally substituted C₁₋₄alkyl, optionally substituted C₂₋₄alkynyl, optionally substituted C₁₋₄alkylnitrile, optionally substituted haloC₁₋₄alkyl, optionally substituted C₃₋₆cycloalkyl, optionally substituted C₁alkylC₃₋₆cycloalkyl, optionally substituted aryl, optionally substituted haloaryl, optionally substituted C₁alkylaryl, optionally substituted haloC₁alkylaryl, optionally substituted haloC₁alkoxyaryl, optionally substituted benzyl, optionally substituted halobenzyl, optionally substituted C₁alkylbenzyl, optionally substituted C₁alkoybenzyl and optionally substituted haloC₁alkoybenzyl.

In some embodiments, X is selected from an optionally substituted C₁₋₄alkyl, an optionally substituted haloC₁₋₄alkyl and a C₃₋₆cycloalkyl.

In some embodiments, X is selected from an optionally substituted C₁₋₂alkyl, an optionally substituted haloC₁₋₂alkyl and a C₃cycloalkyl.

In some embodiments, X is an optionally substituted haloC₁₋₄alkyl selected from —CHF₂, —CF₃, —CH₂CF₃, —CH₂CHF₂ and —CH₂CH₂CF₃.

In some embodiments, X is an optionally substituted haloC₁₋₂alkyl.

In some embodiments, X is an optionally substituted amino preferably disubstituted amino, such as —N(C₁₋₄alkyl)₂. In some embodiments, X is —N(CH₃)₂.

In some embodiments, X is selected from any one of the following groups:
methyl, ethyl, isopropyl, tert-butyl, —CHF₂, —CF₃, —CH₂CF₃, —CH₂CHF₂, —CH₂CH₂CF₃, —CH₂CH₂OCH₃, —CH₂CH₂NH₂, —CH₂CH₂N(CH₃)₂, cyclohexyl, cyclopropyl, —N(CH₃)₂

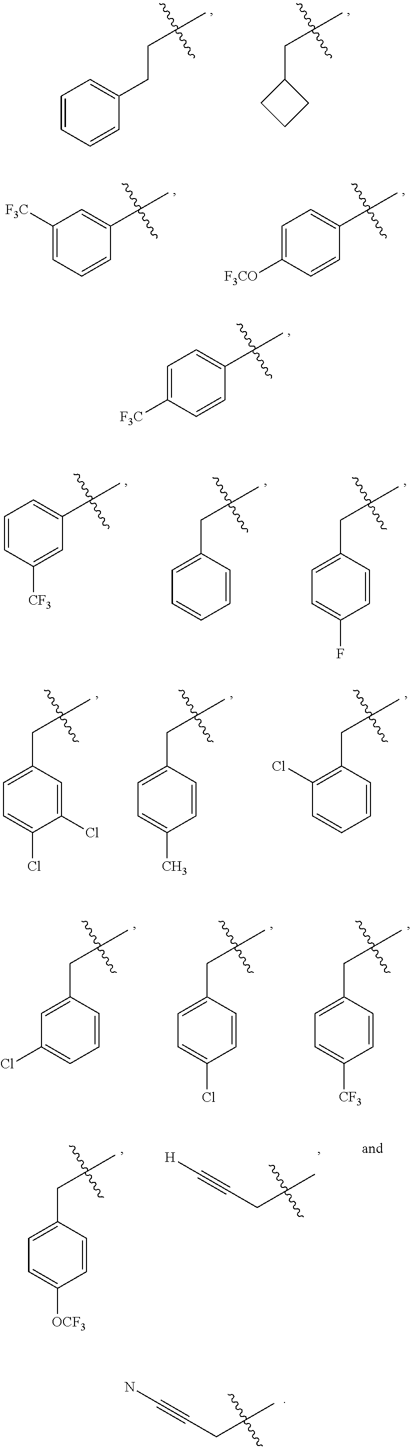

In some embodiments, X is selected from any one of the following groups: ethyl, difluoromethyl, trifluoroethyl and cyclopropyl.

In some embodiments, X is difluoromethyl.

In some embodiments, X is a group that has a longest linear chain extending from the sulfur atom depicted in formula (I) by not more than 6, 5, 4, 3 or 2 atoms, preferably 3-6 atoms. By “longest linear chain” it is meant the number of atoms from the point of attachment not including any branching or rings. For example, when X is benzyl, the longest linear chain is 6 atoms which includes the methylene carbon atom, four ring atoms and the hydrogen atom attached to the carbon at the 4-position of the benzyl, and when X is —CH₂CF₃, the longest linear chain is 3. The longest linear chain in each of these exemplary X-substituents is numbered in the partial formulas shown below:

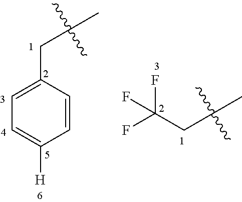 [SMILES: *Cc1ccccc1 ; *CC(F)(F)F]

### Y and Z

In some embodiments, Y and Z are independently selected from H, R⁴, —OR⁴ and —NR⁴R⁴;
wherein at least one of Y and Z is H.

In some embodiments, Y and Z are independently selected from H, R⁴, —OR⁴ and halo;
wherein at least one of Y and Z is H.

In some embodiments, Y and Z are independently selected from H, R⁴, —OR⁴, —NR⁴R⁴ and halo;
wherein at least one of Y and Z is H and the remaining one of Y and Z is selected from R⁴, —OR⁴, —NR⁴R⁴ and halo.

In some embodiments, Y and Z are independently selected from H, R⁴, —OR⁴ and halo;
wherein at least one of Y and Z is H and the remaining one of Y and Z is selected from R⁴, —OR⁴ and halo.

In some embodiments, Y and Z are independently selected from H, R⁴, —OR⁴;
wherein at least one of Y and Z is H.

In some embodiments, Y and Z are independently selected from H, —OR⁴; and halo;
wherein at least one of Y and Z is H.

In some embodiments, Y and Z are independently selected from H and —OR⁴;
wherein at least one of Y and Z is H.

In some embodiments, when Y is —OR⁴; Z is H. In these embodiments, the compound of formula (I) may be provided as a compound of formula (Ia):

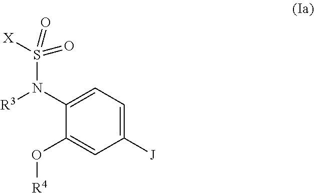

wherein J, A¹, A², A³, A⁴, A⁵, A⁶, n, R¹, R², R³, R⁴, R⁵ and X are as defined for formula (I) or any embodiment thereof.

In some embodiments, when Z is —OR⁴; Y is H. In these embodiments, the compound of formula (I) may be provided as a compound of formula (Ib):

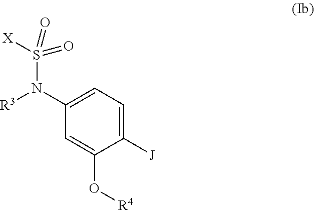

wherein J, A¹, A², A³, A⁴, A⁵, A⁶, n, R¹, R², R³, R⁴, R⁵ and X are as defined for formula (I) or any embodiment thereof.

In some embodiments, when Z is —R⁴; Y is H.

In some embodiments, when Y is —R⁴; Z is H.

In some embodiments, when Z is halo; Y is H.

In some embodiments, when Y is halo; Z is H.

In some embodiments, both of Y and Z are H.

In some embodiments, Y and Z are independently selected from H, R⁴, —OR⁴ and halo;
wherein at least one of Y and Z is H and the remaining one of Y and Z is selected from R⁴, —OR⁴ and halo.

In some embodiments, Y and Z are independently selected from H, R⁴ and —OR⁴;
wherein at least one of Y and Z is H and the remaining one of Y and Z is selected from R⁴, and —OR⁴.

In some embodiments, Y and Z are independently selected from H, —OR⁴ and halo;
wherein at least one of Y and Z is H and the remaining one of Y and Z is selected from —OR⁴ and halo.

In some embodiments, the halo at Y or Z is fluoro.

### R^{4′}

In some embodiments, R⁴ is H. In some embodiments, R⁴ is C₁₋₆alkyl.

### R⁴

In some embodiments, R⁴ is selected from C₁₋₆alkyl, aryl, cycloalkyl, heterocyclyl, C₁₋₆alkylcycloalkyl, C₁₋₆alkylaryl and C₁₋₆alkylheterocyclyl,
wherein each alkyl (including when present as an optional substituent) is optionally substituted with one or more groups independently selected from halo, C₁₋₄alkoxy, hydroxy, nitrile, amino, C₁₋₄alkylamino, (C₁₋₄alkyl)₂amino, aryl, cycloalkyl and heterocyclyl;
wherein each aryl (including when present as an optional substituent) is optionally substituted with one or more groups independently selected from halo, hydroxy, nitrile, amino, C₁₋₄ alkylamino and (C₁₋₄alkyl)₂amino, C₁₋₄alkyl, C₁₋₄alkoxy, haloC₁₋₄alkyl, haloC₁₋₄alkoxy, aryl, cycloalkyl and heterocyclyl;
wherein each cycloalkyl (including when present as an optional substituent) is optionally substituted with one or more groups independently selected from halo, hydroxy, nitrile, amino, C₁₋₄alkylamino and (C₁₋₄alkyl)₂amino, C₁₋₄alkyl, C₁₋₄alkoxy, haloC₁₋₄alkyl, haloC₁₋₄alkoxy, aryl, cycloalkyl and heterocyclyl; and
wherein each heterocyclyl (including when present as an optional substituent) is optionally substituted with one or more groups independently selected from halo, hydroxy, nitrile, amino, C₁₋₄alkylamino and (C₁₋₄alkyl)₂amino, C₁₋₄alkyl, C₁₋₄alkoxy, haloC₁₋₄alkyl, haloC₁₋₄alkoxy, cycloalkyl, heterocyclyl and aryl.

In some embodiments, R⁴ is selected from C₁₋₆alkyl, aryl, cycloalkyl, heterocyclyl, C₁₋₆ alkylcycloalkyl, C₁₋₆alkylaryl, C₁₋₆alkylheterocyclyl, C₃₋₁₀cycloalkylaryl, C₃₋₁₀cycloalkylheterocyclyl, C₃₋₁₀cycloalkylC₃₋₁₀cycloalkyl, 3-6 membered non-aromatic heterocyclyl-aryl, 3-6 membered non-aromatic heterocyclyl-C₃₋₁₀cycloalkyl and 3-6 membered non-aromatic heterocyclyl-3-10 membered heterocyclyl and wherein each cycloalkyl, aryl and heterocyclyl are optionally substituted with one or more groups independently selected from halo, hydroxy, nitrile, amino, C₁₋₄alkylamino and (C₁₋₄alkyl)₂amino, C₁₋₄alkyl, C₁₋₄alkoxy, haloC₁₋₄alkyl and haloC₁₋₄alkoxy.

In some embodiments, R⁴ is selected from C₁₋₆alkyl, aryl, cycloalkyl, heterocyclyl and —(CH₂)ₘR⁹,
R⁹ is selected from C₃₋₁₀cycloalkyl, aryl, heterocyclyl, and
m is an integer selected from 1 to 6,
wherein each cycloalkyl, aryl and heterocyclyl are optionally substituted with one or more groups independently selected from halo, C₁₋₄alkyl, C₁₋₄alkoxy, haloC₁₋₄alkyl and haloC₁₋₄alkoxy.

In some embodiments, R⁴ is selected from C₁₋₄alkyl, cycloalkyl, haloaryl, —C₁₋₂alkylaryl, —C₁₋₂ alkylarylhalo, —C₁₋₂alkylC₃₋₆cycloalkyl, —C₁₋₂alkylheterocyclyl, —C₁₋₂alkylarylC₁alkylhalo, —C₁₋₂alkylarylhaloC₁alkyl, —C₁₋₂alkylaralkylhalo, —C₁₋₂alkylarylhaloalkoxy, cycloalkylaryl, cycloalkylheterocyclyl, cycloalkylcycloalkyl, 3-6 membered non-aromatic heterocyclyl-aryl, 3-6 membered non-aromatic heterocyclylcycloalkyl and 3-6 membered non-aromatic heterocyclyl-3-10 membered heterocyclyl wherein each alkyl, cycloalkyl, aryl, aralkyl, heterocyclyl and alkoxyl is optionally substituted with a group selected from halo, hydroxy, nitrile, amino, C₁₋₄alkylamino and (C₁₋₄alkyl)₂amino, C₁₋₄alkyl, C₁₋₄alkoxy, haloC₁₋₄alkyl, haloC₁₋₄alkoxy and acyl.

In some embodiments, R⁴ is an optionally substituted C₁alkylC₆aryl. In some embodiments, the C₁alkyl moiety is substituted. In some embodiments, the aryl moiety is substituted. In some embodiments, the C₁alkylC₆aryl moiety may be represented by the following partial formula:

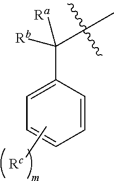

wherein
R¹ and R^{b} are independently selected from H, optionally substituted C₁₋₄alkyl, optionally substituted C₁₋₄alkoxy, optionally substituted C₁₋₄alkylhydroxy, optionally substituted C₁₋₄ alkylnitrile, optionally substituted amino, optionally substituted C₁₋₄alkylamino and optionally substituted (C₁₋₄alkyl)₂amino, or
R^{a} and R^{b} together with the carbon atom to which they are attached form an optionally substituted C₃₋₆cycloalkyl or a 3-6 membered non-aromatic heterocyclyl;
R^{c} is selected from halo and an optionally substituted C₁₋₄alkyl; and
m is 0, 1 or 2.

In some embodiments, R^{a} and R^{b} are independently selected from H, optionally substituted C₁₋₄alkyl, optionally substituted C₁₋₄alkoxy, optionally substituted C₁₋₄alkylhydroxy, optionally substituted C₁₋₄alkylnitrile, optionally substituted C₁₋₄alkylamino and optionally substituted (C₁₋₄alkyl)₂amino. When R^{a} and/or R^{b} are an optionally substituted C₁₋₄alkylamino, either the C₁₋₄alkyl or amino moiety may be optionally substituted.

In some embodiments, R^{a} and R^{b} together with the carbon atom to which they are attached form an optionally substituted C₃₋₆cycloalkyl or a 3-6 membered non-aromatic heterocyclyl selected from an optionally substituted cyclopropyl, an optionally substituted cyclobutyl, an optionally substituted cyclopentyl, an optionally substituted cyclohexyl, an optionally substituted oxetane and an optionally substituted azetidine.

In some embodiments, R^{a} and R^{b} together with the carbon atom to which they are attached form a 3-6 membered non-aromatic heterocyclyl comprising 1 or 2, preferably 1 heteroatom, preferably selected from 0 and N.

In some embodiments, R⁵ is selected from H, C₁₋₆alkyl, C₃₋₈cycloalkyl and aryl. In some embodiments, R⁵ is selected from H and optionally substituted C₁₋₆alkyl. In some embodiments, R⁵ is selected from H and optionally substituted aryl. In some embodiments, R⁵ is selected from H, optionally substituted methyl and optionally substituted phenyl. In some embodiments, R⁵ is H.

In some embodiments, m is 0 or 1.

In some embodiments, m is 1 or 2.

In some embodiments, at least one R^{c} is in the para position relative to the benzyl carbon atom.

In some embodiments, R^{c} is selected from methyl, fluoro and chloro.

In some embodiments, R^{a} is selected from H and methyl, and R^{b} is H.

In some embodiments, R^{a} and R^{b} together with the carbon atom to which they are attached are cyclopropyl.

In some embodiments, R⁴ has partial structure (A):

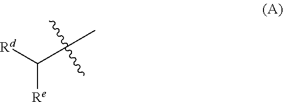

wherein
R^{d} is selected from H, optionally substituted C₁₋₄alkyl, optionally substituted C₁₋₄alkoxy, optionally substituted C₁₋₄alkoxyC₁₋₄alkyl, optionally substituted C₁₋₄alkylhydroxy, optionally substituted C₁₋₄alkylnitrile, optionally substituted C₁₋₄alkylamino and optionally substituted (C₁₋₄alkyl)₂amino, optionally substituted cycloalkyl and optionally substituted C₁₋₄ alkylcycloalkyl; and
R^{e} is selected from optionally substituted aryl, optionally substituted C₁₋₅alkylaryl, optionally substituted heterocyclyl, optionally substituted C₁₋₅alkylheterocyclyl, optionally substituted cycloalkyl, and optionally substituted C₁₋₅alkylC₃₋₁₀cycloalkyl.

In these embodiments, the compound of formula (I) may be provided as a compound of formula (IV):

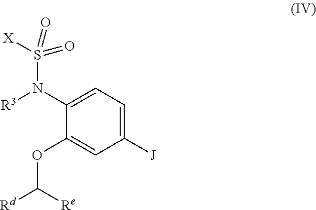

wherein X, R³, R^{d}, R^{e}, and J are as defined for any compound herein.

In some embodiments, R^{d} is methyl.

In some embodiments, R^{e} is selected from optionally substituted aryl, optionally substituted C₁₋₅alkylaryl, optionally substituted heterocyclyl, optionally substituted C₁₋₄alkylheterocyclyl, optionally substituted cycloalkyl, and optionally substituted C₁₋₄alkylC₃₋₁₀cycloalkyl.

In some embodiments, R^{e} is selected from optionally substituted aryl, optionally substituted cycloalkyl and optionally substituted heterocyclyl.

In some embodiments, R^{e} is selected from optionally substituted aryl and optionally substituted heteroaryl.

In some embodiments, R^{d} is selected from optionally substituted C₁₋₄alkyl, optionally substituted cycloalkyl and optionally substituted C₁₋₄alkylcycloalkyl. In these embodiments, where R^{d} and R^{e} are not the same group, the partial structure (A) may contain a chiral centre at the carbon to which R^{d} and R^{e} are attached. Therefore, the carbon atom to which R^{d} and R^{e} are attached may be enantiomerically enriched. In some embodiments, the carbon atom to which R^{d} and R^{e} is attached is enriched as the (S) stereoisomer, for example when R^{e} has a higher ranking than R^{d} in the Cahn-Ingold-Prelog rules for stereochemical assignment. In some embodiments, the carbon atom to which R^{d} and R^{e} is attached is enriched as the (R) stereoisomer, for example when R^{e} has a lower ranking than R^{d} the Cahn-Ingold-Prelog rules for stereochemical assignment. In some embodiments, R^{d} is selected from optionally substituted C₁₋₄alkyl, and the carbon atom to which R^{d} and R^{e} are attached is enriched in the (S) stereoisomer.

In some embodiments, partial structure (A) may have the stereochemical configuration shown in by partial structure (A1):

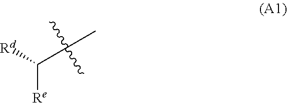

wherein R^{e} has a higher ranking than R^{d} in the Cahn-Ingold-Prelog rules for stereochemical assignment. The inventors have surprisingly found that compounds with this configuration at this position possess greater MLKL activity than those with other configuration(s). In some cases, when R^{e} has a higher ranking than R^{d} in the Cahn-Ingold-Prelog rules for stereochemical assignment MLKL activity may be greater than 2-fold more active than the other corresponding stereoisomer, and in some embodiments, may be at least about 5-fold or about 10-fold more active than other corresponding stereoisomer(s) for MLKL inhibition.

In some embodiments, the compound is provided as a compound of formula (S):

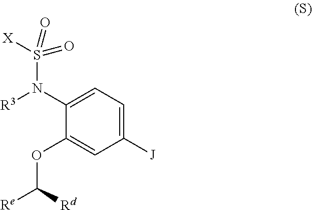

wherein X, J and R³ are as defined for formula (I) and R^{e} and R^{d} are as defined for partial formula (A).

In some embodiments. R⁴ is selected from any one of the following groups:

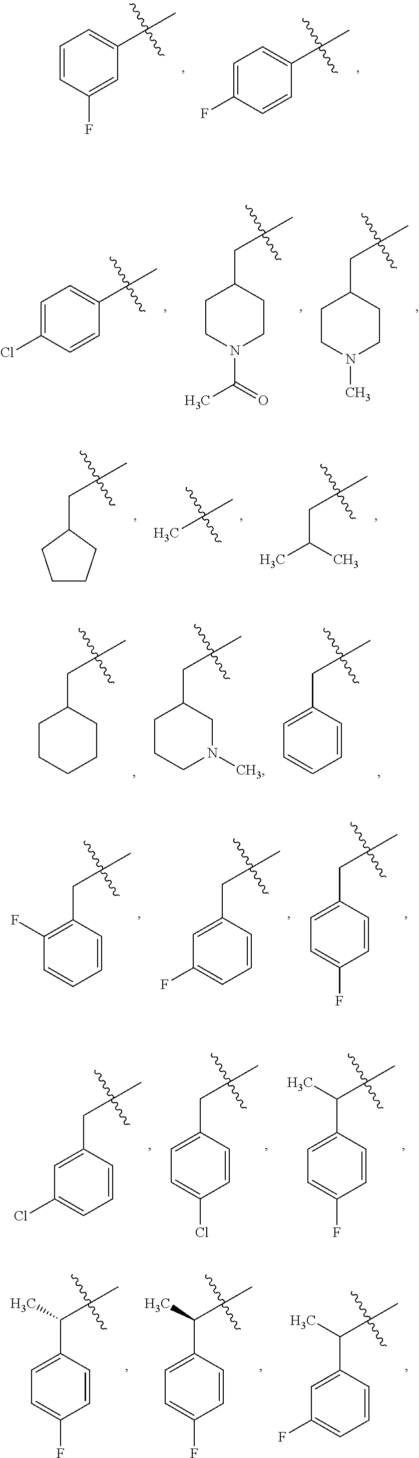

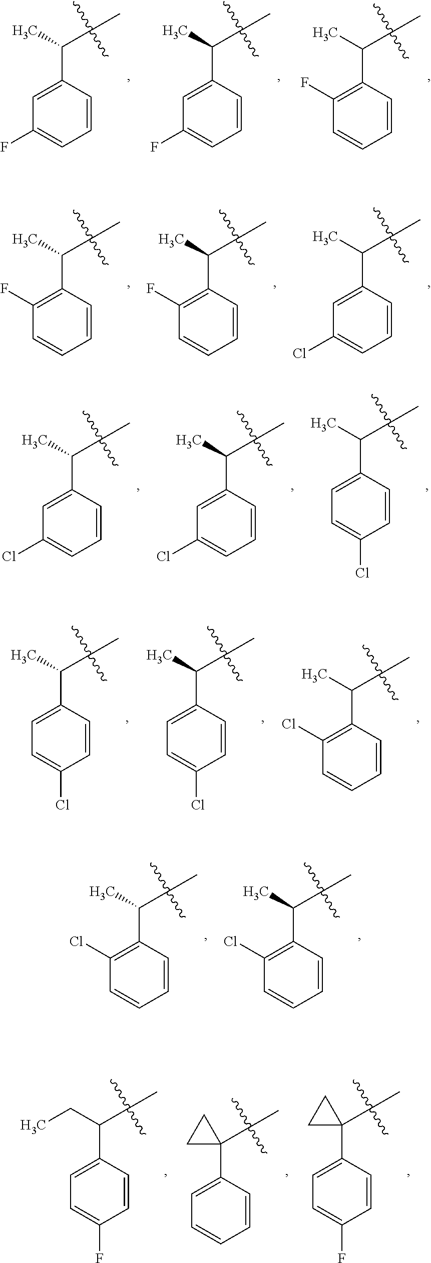

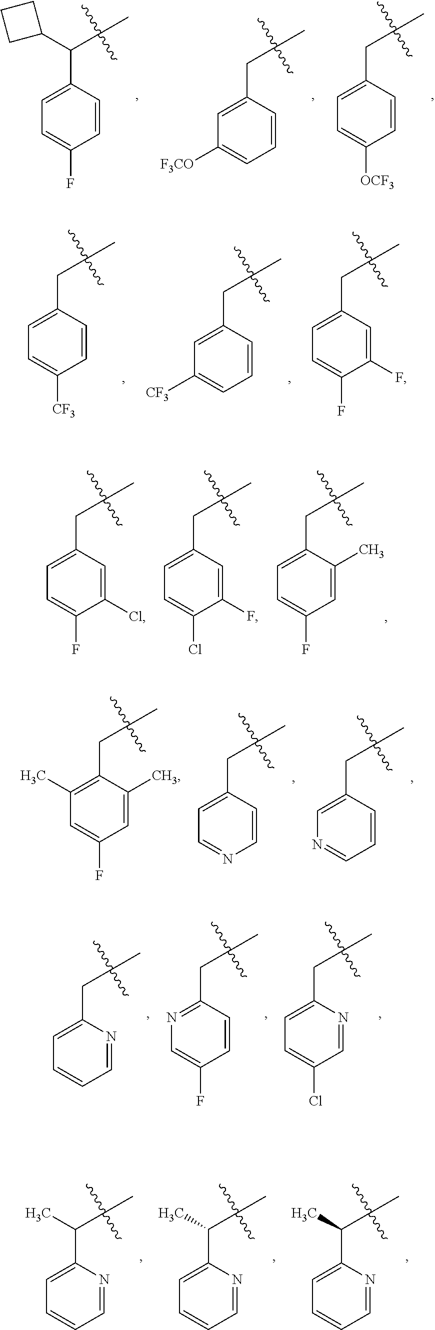

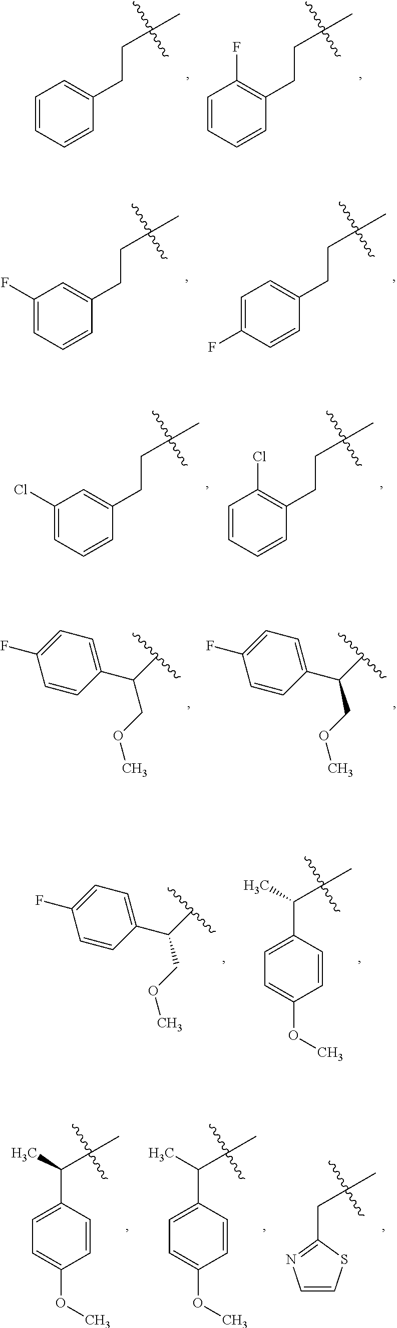

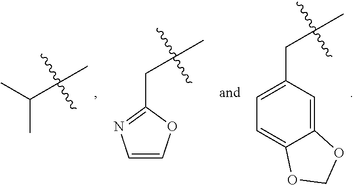

In some embodiments, R⁴ is selected from any one of the following groups:

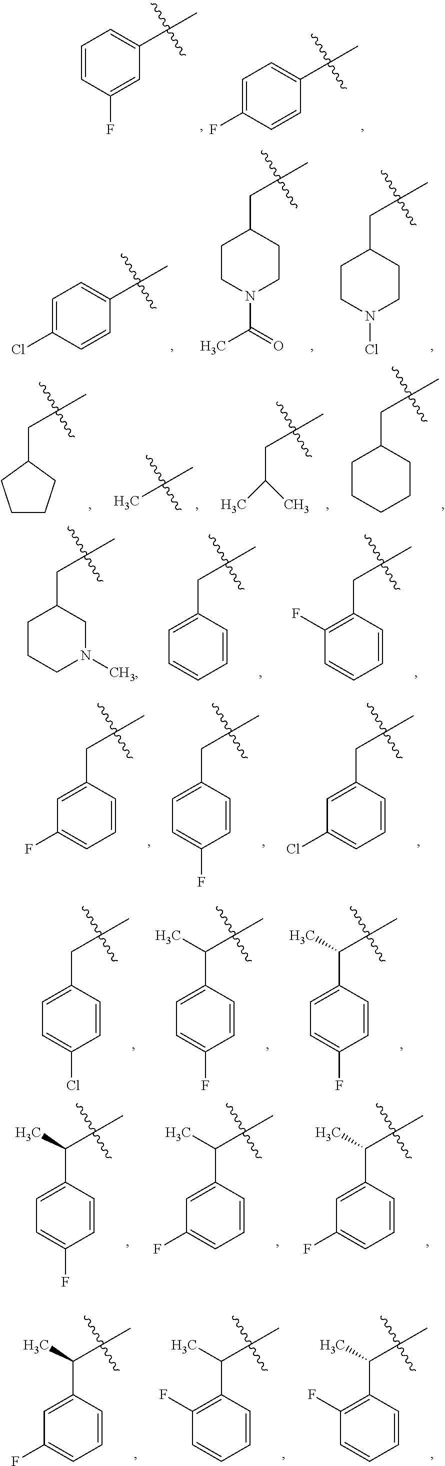

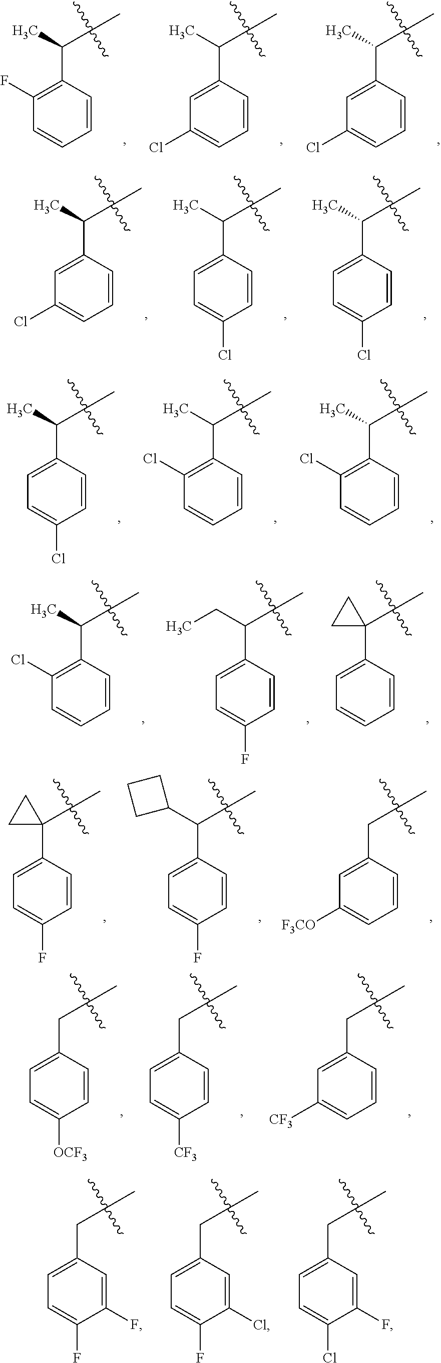

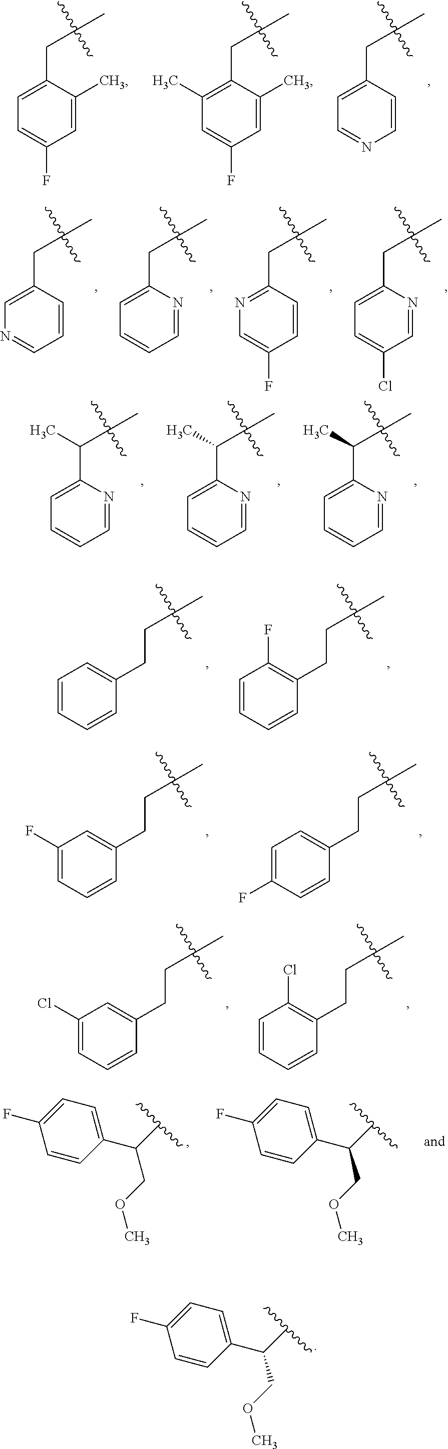

In some embodiments, R⁴ is selected from any one of the following groups:

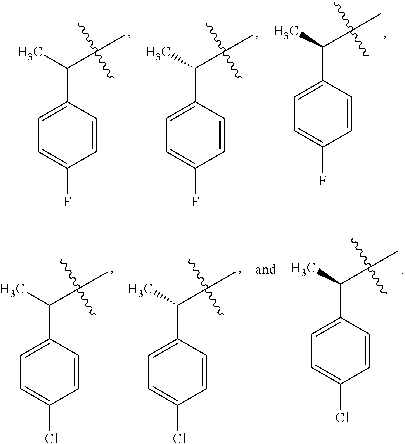

In some embodiments, R⁴ is selected from any one of the following groups:

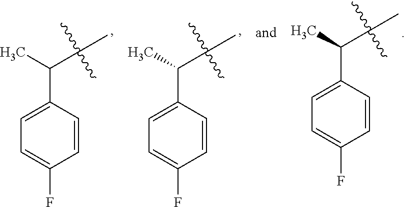

In some embodiments, R⁴ is:

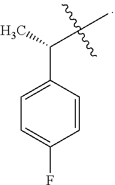 [SMILES: *[C@@H](C)c1ccc(F)cc1]

R¹ and R³

In some embodiments, R¹ and R³ are H.

In some embodiments, R³ is H and R¹ is selected from H, methyl and isopropyl, preferably methyl.

In some embodiments, R¹ is selected from H, methyl and isopropyl. In some embodiments, R¹ is methyl.

### J1

In some embodiments, J is J1. In these embodiments, the compound of formula (I) may be provided as a compound of formula (II)

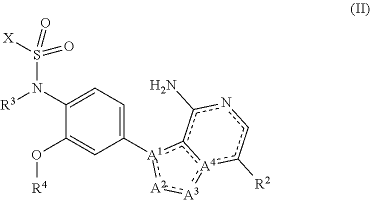

wherein X, R², R³, R⁴, A¹, A², A³ and A⁴ are as defined for formula (I) or any embodiment thereof.

In embodiments where J is J1, the compound of formula (I) may also be provided as a compound of formula (IIe)

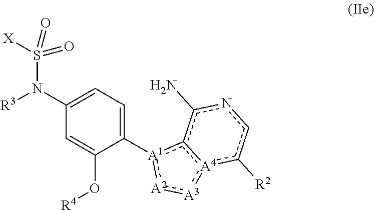

wherein X, R², R³, R⁴, A¹, A², A³ and A⁴ are as defined for formula (I) or any embodiment thereof.

The J1 structure, as shown below (and equivalent structures within other formulas), indicates a conjugated fused aromatic ring system:

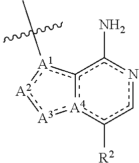

For instance, in some embodiments J1 may have the following substitution pattern:

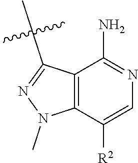

Similarly, in other embodiments J1 may have the following substitution pattern:

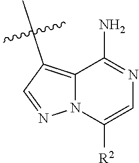

In compounds wherein J is J1, at least one of A¹, A², A³ and A⁴ is selected from N, NR¹, O and S. In some embodiments, A³ is selected from N, NR¹, O and S.

In some embodiments, A¹ is C or N.

In some embodiments, A¹ is C.

In some embodiments, A² is selected from N and CH.

In some embodiments, A⁴ is selected from C and N.

In some embodiments, A¹, A², A³ and A⁴ are selected from the following embodiments:

| No. | A¹ | A² | A³ | A⁴ |
|---|---|---|---|---|
| 1 | C | CH | O | C |
| 2 | C | N | NMe | C |
| 3 | C | CH | N | N |
| 4 | C | N | NⁱPr | C |
| 5 | C | N | CH | N |
| 6 | C | N | S | C |
| 7 | C | CH | S | C |
| 8 | N | CH | N | C |

In some embodiments, any one of embodiment nos. 1-4 are preferred.

In some embodiments, embodiment no. 2 is preferred.

### R²

In some embodiments, R² is selected from:
(i) H
(ii) optionally substituted C₁₋₄alkylamido,
(iii) optionally substituted C₁₋₄alkylaryl,
(iv) optionally substituted C₂₋₄alkynyl,
(v) optionally substituted aryl,
(vi) optionally substituted 5- or 6-membered heterocyclyl.

In some embodiments, R² is selected from:
(i) optionally substituted C₁₋₄alkylamido,
(ii) optionally substituted C₁₋₄alkylaryl,
(iii) optionally substituted C₂₋₄alkynyl,
(iv) optionally substituted aryl,
(v) optionally substituted 5- or 6-membered heterocyclyl.

In some embodiments, R² is selected from optionally substituted C₂₋₄alkynyl and optionally substituted 5- or 6-membered heterocyclyl. In some embodiments, R² is selected from optionally substituted C₂₋₄alkynyl and optionally substituted 5- or 6-membered aromatic heterocyclyl. In some embodiments, R² is selected from optionally substituted C₂₋₄alkynyl and optionally substituted 5-membered aromatic heterocyclyl. In some embodiments, R² is selected from optionally substituted C₂₋₄alkynyl and optionally substituted 5- or 6-membered heterocyclyl, wherein the ring heteroatom(s) of the heterocyclyl are N. In some embodiments, R² is selected from optionally substituted C₂₋₄alkynyl and optionally substituted 5- or 6-membered aromatic heterocyclyl, wherein the ring heteroatom(s) of the heterocyclyl are N.

In some embodiments, R² is optionally substituted with one or more group(s) (preferably 1-3 groups) selected from: halo, an optionally substituted C₁₋₆alkyl, an optionally substituted C₃₋₈cycloalkyl, an optionally substituted C₁₋₆alkyl-N(R¹¹)₂, —N(R¹¹)₂, an optionally substituted (C₁₋₆alkyl)₂amino, an optionally substituted aryl, an optionally substituted heterocyclyl, an optionally substituted haloheterocyclyl, an optionally substituted C₁₋₆alkylheterocyclyl, an optionally substituted haloC₁₋₆alkylheterocyclyl, an optionally substituted C₁₋₄alkoxyheterocyclyl, an optionally substituted acylheterocyclyl, an optionally substituted C₁₋₄alkoxy, an optionally substituted C₁₋₄alkyl-OH, an optionally substituted C₁₋₄alkylhalo, an optionally substituted C₁₋₄alkylheterocyclyl, an optionally substituted C₁₋₄alkylC₃₋₈cycloalkyl, an optionally substituted C₁₋₄alkylaryl, wherein each R¹¹ is independently selected from H, optionally substituted C₁₋₄alkyl and optionally substituted C₁₋₄alkylhalo, or 2 R¹¹ together with the nitrogen atom to which they are attached form a 3-8 membered heterocyclyl.

In some embodiments, R² is a 5- or 6-membered heterocyclyl optionally substituted with one or more group(s) selected from: halo, an optionally substituted C₁₋₆alkyl, an optionally substituted C₃₋₈cycloalkyl, an optionally substituted C₁₋₆alkyl-N(R¹¹)₂, —N(R¹¹)₂, an optionally substituted (C₁₋₆alkyl)₂amino, an optionally substituted aryl, an optionally substituted heterocyclyl, an optionally substituted haloheterocyclyl, an optionally substituted C₁₋₆ alkylheterocyclyl, an optionally substituted haloC₁₋₆alkylheterocyclyl, an optionally substituted C₁₋₄alkoxyheterocyclyl, an optionally substituted acylheterocyclyl, an optionally substituted C₁₋₄alkoxy, an optionally substituted C₁₋₄alkyl-OH, an optionally substituted C₁₋₄alkylhalo, an optionally substituted C₁₋₄alkylheterocyclyl, an optionally substituted C₁₋₄alkylC₃₋₈cycloalkyl, an optionally substituted C₁₋₄alkylaryl, wherein each R¹¹ is independently selected from H, optionally substituted C₁₋₄alkyl and optionally substituted C₁₋₄alkylhalo, or 2 R¹¹ together with the nitrogen atom to which they are attached form a 3-8 membered heterocyclyl.

In some embodiments, R² is a 5- or 6-membered heterocyclyl optionally substituted with one or more group(s) selected from: halo, an optionally substituted C₁₋₆alkyl, an optionally substituted C₃₋₈cycloalkyl, an optionally substituted aryl, an optionally substituted heterocyclyl, an optionally substituted C₁₋₄alkoxy, an optionally substituted C₁₋₄alkyl-OH, an optionally substituted C₁₋₄alkylhalo, an optionally substituted C₁₋₄alkylheterocyclyl, an optionally substituted C₁₋₄alkylC₃₋₈cycloalkyl, an optionally substituted C₁₋₄alkylaryl;

In some embodiments, R² is H.

In some embodiments, R² is cyano.

In some embodiments, R² is H or cyano.

In some embodiments, R² is optionally substituted C₁₋₄alkylamido or optionally substituted C₂₋₄alkynyl.

In some embodiments, R² is selected from optionally substituted C₁₋₄alkylaryl, optionally substituted aryl, and optionally substituted 5- or 6-membered heterocyclyl.

In some embodiments, R² is selected from:
(i) optionally substituted aryl,
(ii) a 5- or 6-membered heterocyclyl optionally substituted with one or more group(s) selected from: halo, an optionally substituted C₁₋₆alkyl, an optionally substituted C₃₋₈cycloalkyl, an optionally substituted aryl, an optionally substituted heterocyclyl, an optionally substituted C₁₋₄alkoxy, an optionally substituted C₁₋₄alkyl-OH, an optionally substituted C₁₋₄alkylhalo, an optionally substituted C₁₋₄alkylheterocyclyl, an optionally substituted C₁₋₄alkylC₃₋₈cycloalkyl, an optionally substituted C₁₋₄alkylaryl;

In some embodiments, R² is a 5- or 6-membered heterocylyl selected from: pyrazolyl, pyridyl, tetrahydropyridyl, isozazolyl, pyrimidinyl, piperidinyl and tetrahydropyranyl, and wherein the 5- or 6-membered heterocyclyl is optionally substituted with one or more group(s) selected from: halo, an optionally substituted C₁₋₆alkyl, an optionally substituted C₃₋₈cycloalkyl, an optionally substituted C₁₋₆alkyl-N(R¹¹)₂, —N(R¹¹)₂, an optionally substituted (C₁₋₆alkyl)₂amino, an optionally substituted aryl, an optionally substituted heterocyclyl, an optionally substituted haloheterocyclyl, an optionally substituted C₁₋₆ alkylheterocyclyl, an optionally substituted haloC₁₋₆alkylheterocyclyl, an optionally substituted C₁₋₄alkoxyheterocyclyl, an optionally substituted acylheterocyclyl, an optionally substituted C₁₋₄alkoxy, an optionally substituted C₁₋₄alkyl-OH, an optionally substituted C₁₋₄alkylhalo, an optionally substituted C₁₋₄alkylheterocyclyl, an optionally substituted C₁₋₄alkylC₃₋₈cycloalkyl, an optionally substituted C₁₋₄alkylaryl, wherein each R¹¹ is independently selected from H, optionally substituted C₁₋₄alkyl and optionally substituted C₁₋₄alkylhalo, or 2 R¹¹ together with the nitrogen atom to which they are attached form a 3-8 membered heterocyclyl.

In some embodiments, R² is a 5- or 6-membered heterocyclyl optionally substituted with one or more substituent(s) selected from: halo, optionally substituted C₁₋₆alkyl, optionally substituted C₁₋₆alkylamido, optionally substituted C₃₋₈cycloalkyl, optionally substituted C₁₋₆ alkylC₃₋₈cycloalkyl, optionally substituted haloC₁₋₆alkylC₃₋₈cycloalkyl, optionally substituted aryl, optionally substituted heterocyclyl, optionally substituted C₁₋₆alkylheterocyclyl, optionally substituted C₁₋₆alkoxy, optionally substituted C₁₋₆alkyl-OH and optionally substituted C₁₋₆ alkylhalo. In some embodiments, the optionally substituted C₁₋₆alkylamido is provided by —C₁₋₆alklC(O)NR′R″, wherein R′ and R″ are independently selected from H and optionally substituted C₁₋₆alkyl. In some embodiments, any heterocyclyl group of these optional substituents may be a 4-7 membered heterocyclyl (including nonaromatic heterocyclyl and heteroaryl groups).

In some embodiments, R² is a 5- or 6-membered heterocyclyl optionally substituted with one, two or three of any of the optional substituent(s) of R² described herein.

In some embodiments, R² is a 5- or 6-membered heterocyclyl optionally substituted with one, two or three groups, preferably one or two groups, selected from: methyl, methoxy, methoxyethyl, trifluoromethyl, difluoromethyl, trifluoroethyl, isopropyl, tert-butyl, 2-hydroxyethyl, dimethylamino, cyclopentyl, oxetanyl, tetrahydrofuranyl, piperidinyl, N-methyl-piperidinyl, tetrahydropyranyl and N-methyl-piperazinyl-2-ethyl.

In some embodiments, R² is an optionally substituted 5- or 6-membered heterocyclyl selected from 4-pyridyl, 3-pyridyl, 4-piperidinyl, 1,4-piperazinyl, 4-tetrahydropyranyl, 2H,4H,5H-3-piperidinyl, 3,4-pyrazolyl, 3H,5H,6H-4-tetrahydropyranyl, 2,4-pyrimidinyl and 3,4-isoxazolyl.

In some embodiments, R² is an optionally substituted pyrazolyl, preferably a substituted pyrazolyl, more preferably a mono-substituted pyrazolyl. Preferably, the mono-substituted pyrazolyl is substituted at N.

In some embodiments, R² is a 5- or 6-membered heterocyclyl optionally substituted with one or more group(s) selected from: optionally substituted C₁₋₄alkylhalo, optionally substituted heterocyclyl, optionally substituted C₁₋₆alkylheterocyclyl and optionally substituted haloC₁₋₆ alkylheterocyclyl. In these embodiments, the 5- or 6-membered heterocyclyl is preferably selected from pyrazolyl, pyridyl, tetrahydropyridyl, isozazolyl, pyrimidinyl, piperidinyl and tetrahydropyranyl, more preferably pyrazolyl, most preferably N-substituted-pyrazolyl.

In some embodiments, R² is a 5- or 6-membered heterocyclyl optionally substituted with one or more group(s) selected from: optionally substituted C₁₋₄alkylhalo, optionally substituted heterocyclyl, optionally substituted C₁₋₆alkylheterocyclyl and optionally substituted haloC₁₋₆ alkylheterocyclyl;
wherein the optionally substituted C₁₋₄alkylhalo, optionally substituted heterocyclyl, optionally substituted C₁₋₆alkylheterocyclyl and optionally substituted haloC₁₋₆ alkylheterocyclyl; are optionally substituted with one or more substituent(s) selected from C₁₋₆alkyl and C₃₋₄cycloalkyl. In these embodiments, the 5- or 6-membered heterocyclyl is preferably selected from pyrazolyl, pyridyl, tetrahydropyridyl, isozazolyl, pyrimidinyl, piperidinyl and tetrahydropyranyl, more preferably pyrazolyl, most preferably N-substituted-pyrazolyl.

In some embodiments, R² is an optionally substituted 6-membered non-aromatic heterocyclyl.

In some embodiments, R² is a 5- or 6-membered heterocyclyl comprising at least 1 nitrogen heteroatom. In some embodiments, R² is a 5- or 6-membered heterocyclyl comprising at least 1 oxygen heteroatom. In some embodiments, R² is a 5- or 6-membered heterocyclyl comprising at least 1 nitrogen and at least 1 oxygen heteroatom. In some embodiments, R² is a 5- or 6-membered heterocyclyl comprising 2 nitrogen heteroatoms.

In some embodiments, R² is an optionally substituted fused heterocyclyl.

In some embodiments, R² is an optionally substituted 5- or 6-membered heteroaryl.

In some embodiments, R² is an optionally substituted pyridyl.

In some embodiments, R² is an optionally substituted isoxazolyl.

In some embodiments, R² is an optionally substituted morpholinyl.

In some embodiments, R² is an optionally substituted pyrimidinyl.

In some embodiments, R² is an optionally substituted pyrrolyl.

In some embodiments, R² is an optionally substituted 1,3-dihydro-2H-benzo[d]imidazol-2-one, preferably including methyl substitution.

In some embodiments, R² is an optionally substituted 1-methylindolinyl-2-one.

In some embodiments, R² is an optionally substituted 1-methyl-1H-indazolyl.

In some embodiments, R² is enriched with one or more of the following minor isotopes: ²H, ³H, ¹³C, ¹⁴C, ¹⁵N and/or¹⁷O, preferably ²H.

In some embodiments, R² is an optionally substituted C₂₋₄alkynyl. The C₂₋₄alkynyl may optionally be substituted with one or more groups (preferably 1 group) selected from optionally substituted C₃₋₈cycloalkyl, optionally substituted haloC₃₋₈cycloalkyl, optionally substituted (C₁₋₆alkyl)₁₋₃C₃₋₈cycloalkyl, optionally substituted heterocyclyl, optionally substituted halo heterocyclyl, optionally substituted (C₁₋₆alkyl)₁₋₃heterocyclyl and optionally substituted (C₁₋₆alkylhalo)₁₋₃heterocyclyl.

In some embodiments, R² is an optionally substituted C₂₋₄alkynyl. The C₂₋₄alkynyl may optionally be substituted with one or more groups (preferably 1 group) selected from optionally substituted C₃₋₈cycloalkyl, optionally substituted haloC₃₋₈cycloalkyl, optionally substituted (C₁₋₆alkyl)₁₋₃C₃₋₈cycloalkyl, optionally substituted heterocyclyl, optionally substituted halo heterocyclyl and optionally substituted (C₁₋₆alkyl)₁₋₃heterocyclyl.

In some embodiments, R² is selected from: H, cyano, methyl, 3-pyridyl, 4-pyridyl, benzyl,

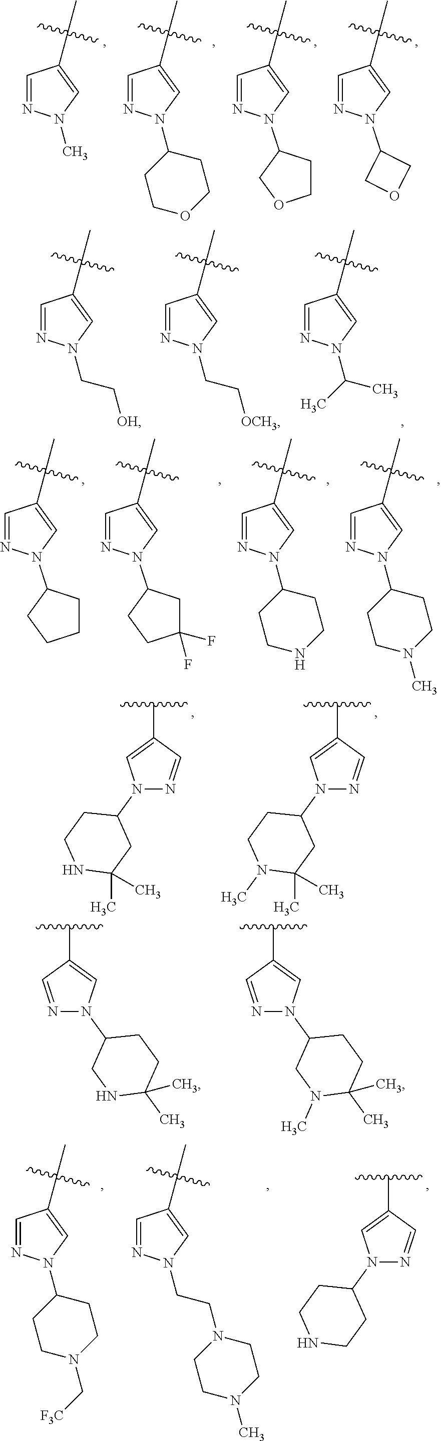

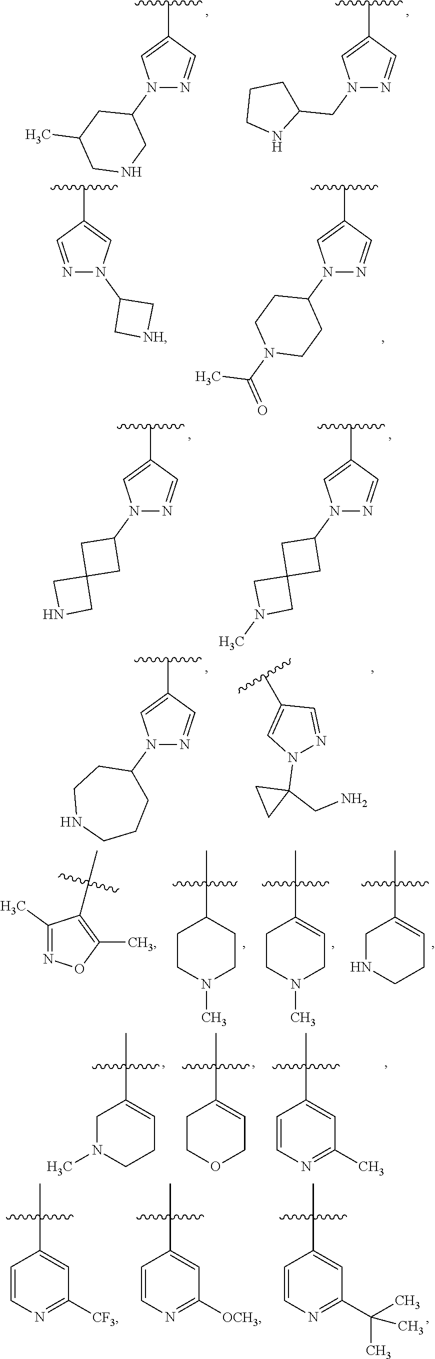

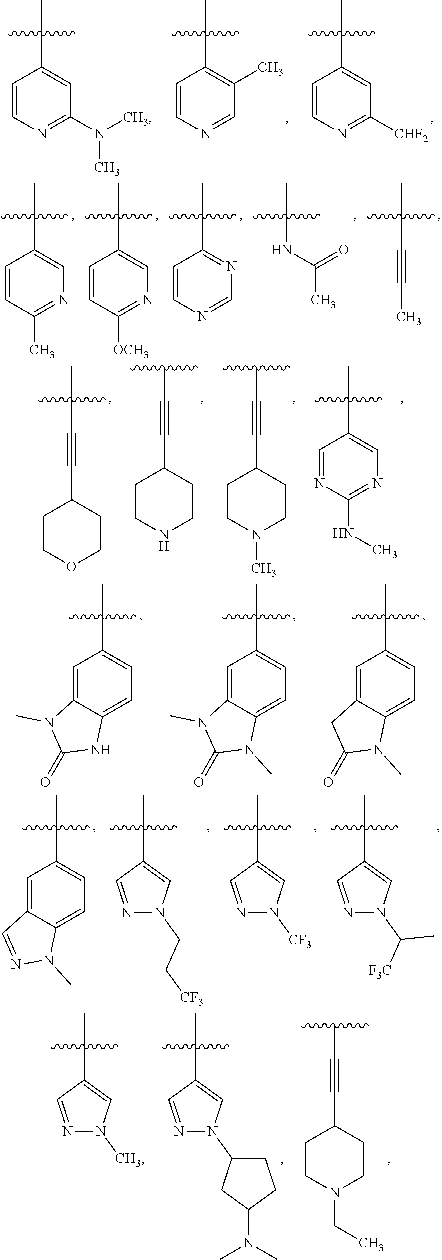

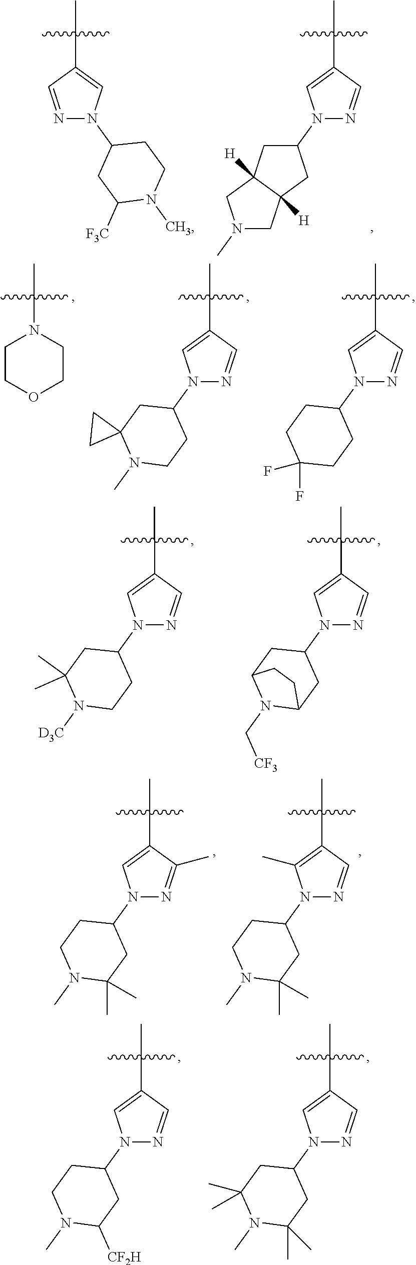

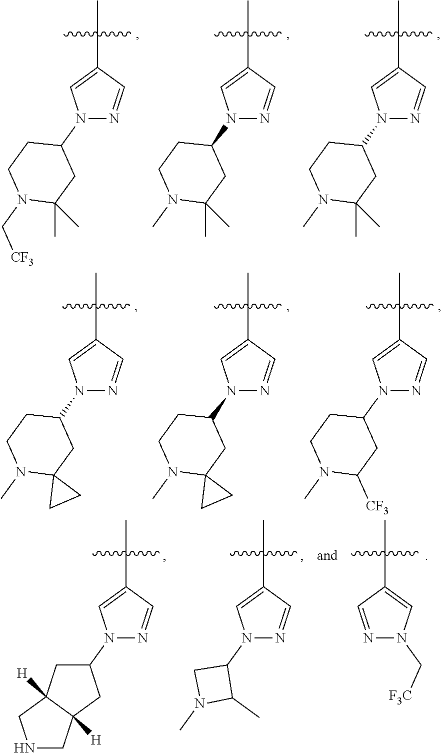

In some embodiments, R² is selected from: H, methyl, 3-pyridyl, 4-pyridyl, benzyl,

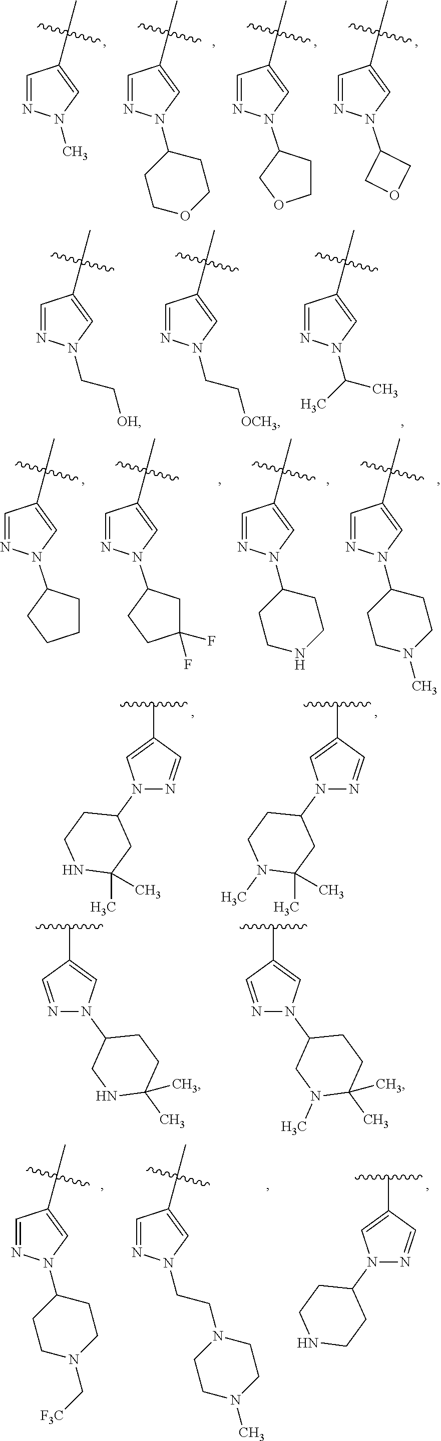

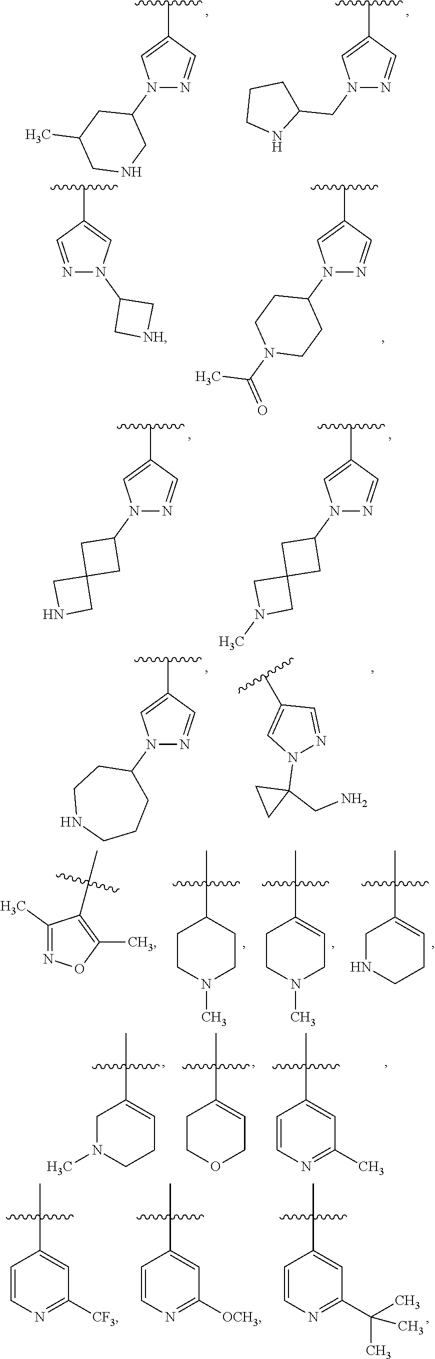

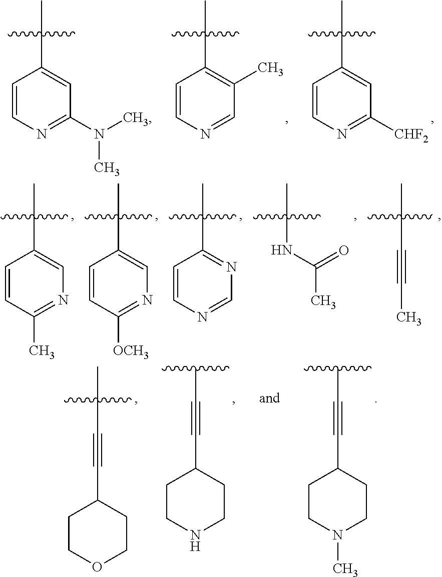

In some embodiments, R² comprises a basic moiety (such as an optionally substituted amine including an optionally substituted cyclic amine).

In some embodiments, R² comprises a nitrogen-containing heterocyclyl and the nitrogen atom of these cyclic amines may be optionally substituted with a group selected from C₁₋₆ alkyl and a substituted ketone (such as acyl).

In some embodiments, R² is represented by the following partial formula:

-G-(R¹⁰)_{w}
wherein
G is selected from:
   (i) C₁₋₄alkylamido,
   (ii) C₁₋₄alkylaryl,
   (iii) C₂₋₄alkynyl,
   (iv) aryl,
   (v) 5- or 6-membered heterocyclyl
each R¹⁰ is independently selected from: halo, an optionally substituted C₁₋₆alkyl, an optionally substituted C₃₋₈cycloalkyl, an optionally substituted aryl, an optionally substituted heterocyclyl, an optionally substituted C₁₋₄alkoxy, an optionally substituted C₁₋₄alkyl-OH, an optionally substituted C₁₋₄alkylhalo, an optionally substituted C₁₋₄alkylheterocyclyl, an optionally substituted C₁₋₄alkylC₃₋₈cycloalkyl, an optionally substituted C₁₋₄alkylaryl; and w is an integer from 0-3.

Accordingly, in some embodiments, the compound is provided as a compound of formula (IIA) or (IIB):

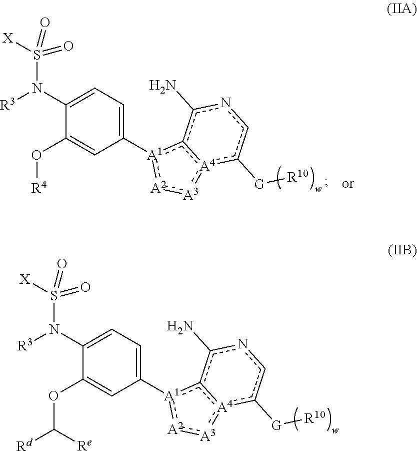

wherein A¹, A², A³, A⁴, R³, R⁴, R^{d}, R^{e}, G, w and R¹⁰ are as defined herein.

In some embodiments, G is a C₂₋₄alkynyl. In some embodiments, G is ethynyl and w is 1.

In some embodiments, G is a 6-membered heterocyclyl. In these embodiments, G may be a 6-membered non-aromatic heterocyclyl or a 6-membered heteroaryl. In these embodiments,
each R¹⁰ may be independently selected from halo, an optionally substituted C₁₋₆alkyl, an optionally substituted C₁₋₄alkoxy, an optionally substituted C₁₋₄alkylhalo and an optionally substituted (C₁₋₆alkyl)₂amine. Typically, in these embodiments, w is 1.

In some embodiments, G is a 6-membered non-aromatic heterocyclyl comprising a heteroatom selected from N and O. When the heteroatom is N, R¹⁰ is preferably bonded to an N ring atom.

In some embodiments, G is a 5-membered heterocyclyl. In these embodiments, G may be a 5-membered non-aromatic heterocyclyl or a 5-membered heteroaryl. In some embodiments, the 5-membered heterocyclyl may comprise heteroatom(s) selected from N and O.

In some embodiments, G is a 5- or 6-membered heterocyclyl comprising at least 1 oxygen heteroatom. In some embodiments, G is a 5- or 6-membered heterocyclyl comprising at least 1 nitrogen and at least 1 oxygen heteroatom. In some embodiments, G is a 5- or 6-membered heterocyclyl comprising 2 nitrogen heteroatoms.

In some embodiments, G is an optionally substituted fused heterocyclyl.

In some embodiments, G is selected from morpholinyl, pyrimidinyl, pyrrolyl, 1,3-dihydro-2H-benzo[d]imidazol-2-one, 1-methylindolinyl-2-one, 1-methyl-1H-indazolyl, pyrazolyl and isoxazolyl.

In some embodiments, G is selected from pyrazolyl and isoxazolyl.

In some embodiments, G is pyrazolyl. In these embodiments, R¹⁰ may be bonded to a nitrogen ring atom. In these embodiments, R² may be represented by the following partial formula:

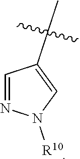

In these embodiments, the compound may be provided as a compound of formula (IIC) or (IID):

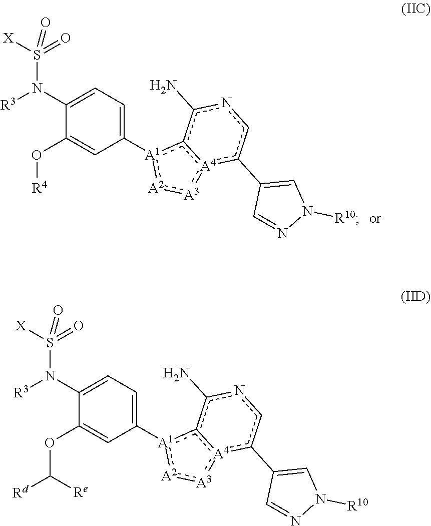

wherein A¹, A², A³, A⁴, R³, R⁴, R^{d}, R^{e} and R¹⁰ are as defined herein.

In some embodiments, R¹⁰ is selected from C₁₋₆alkyl, heterocyclyl, C₁₋₆alkyl-OH, C₁₋₆alkyl-NH₂, C₁₋₆alkoxyC₁₋₆alkyl, C₃₋₈cycloalkyl, wherein the heterocyclyl and C₃₋₈cycloalkyl may be further substituted with one or more groups (preferably 1-3 groups, most preferably 1 group) selected from C₁₋₆alkyl, haloC₁₋₆alkyl, C₁₋₆alkoxy, C₁₋₆alkyl-OH, C₁₋₆alkoxyC₁₋₆alkyl, halo, C₁₋₆alkyl-NH₂, C₁₋₆alkylketone (eg acyl) and —NH₂.

In some embodiments, R¹⁰ is heterocyclyl or alkylheterocyclyl optionally substituted with one or more groups (preferably 1-3 groups, most preferably 1 group) selected from C₁₋₆alkyl, haloC₁₋₆alkyl, C₁₋₆alkoxy, C₁₋₆alkyl-OH, C₁₋₆alkoxyC₁₋₆alkyl, halo, C₁₋₆alkyl-NH₂, C₁₋₆ alkylketone (eg acyl) and —NH₂.

When R¹⁰ is a group comprising a heterocyclyl moiety, the heterocyclyl may preferably be a 4-8 membered heterocyclyl, preferably comprising one heteroatom selected from N and O, preferably N.

In some embodiments, R¹⁰ is an optionally substituted heterocyclyl selected from an optionally substituted spirocyclic heterocyclyl, optionally substituted fused heterocyclyl or an optionally substituted bridged heterocyclyl. In some embodiments, R¹⁰ is an optionally substituted heterocyclyl selected from an optionally substituted spirocyclic heterocyclyl or an optionally substituted bridged heterocyclyl. Preferred optional substituents of the spirocyclic, fused and bridged heterocyclyl groups include C₁₋₆alkyl and haloC₁₋₆alkyl.

In some embodiments, R¹⁰ is enriched with one or more of the following minor isotopes: ²H, ³H, ¹³C, ¹⁴C, ¹⁵N and/or ¹⁷O, preferably ²H

In some embodiments, R¹⁰ is selected from: methyl, difluoromethyl, trifluoromethyl, methoxy, tert-butyl, phenyl, acyl, dimethylamino, tetrahydropyranyl, tetrahydrofuranyl, oxetanyl, hydroxyethyl, methoxyethyl, isopropyl, cyclopentyl, difluorocyclopenyl, piperidinyl, N-methylpiperidinyl, N-acetylpiperidine, azetidinyl,

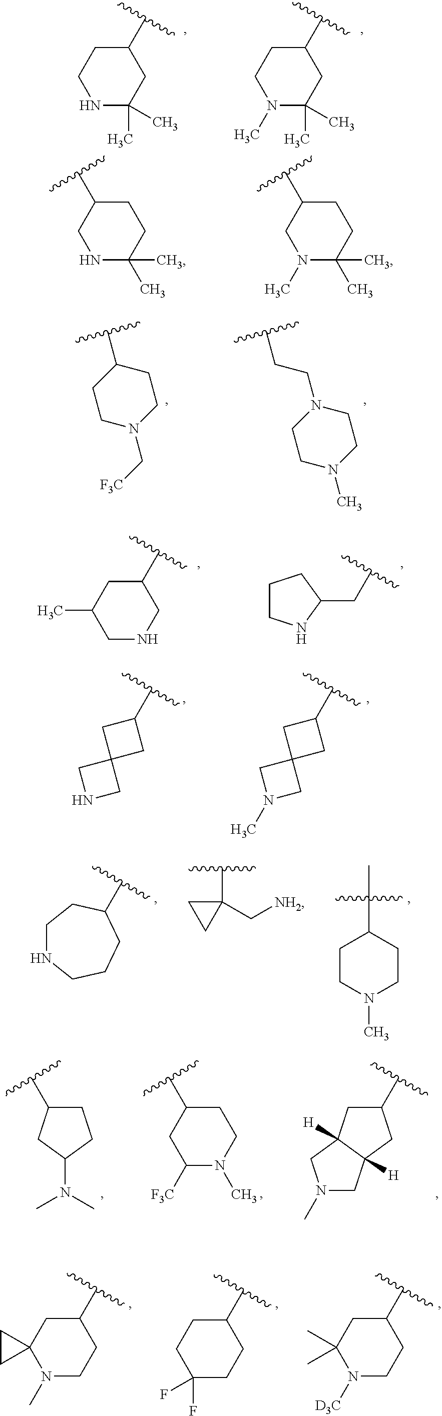

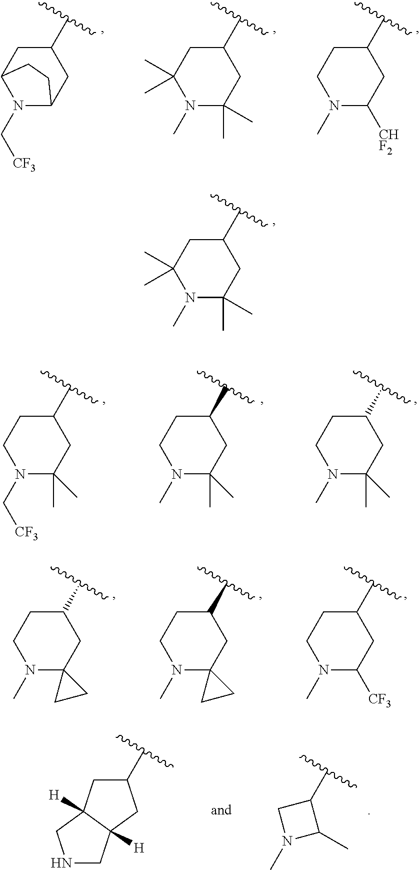

In some embodiments, R¹⁰ is selected from: methyl, difluoromethyl, trifluoromethyl, methoxy, tert-butyl, phenyl, acyl, dimethylamino, tetrahydropyranyl, tetrahydrofuranyl, oxetanyl, hydroxyethyl, methoxyethyl, isopropyl, cyclopentyl, difluorocyclopenyl, piperidinyl, N-methylpiperidinyl, N-acetylpiperidine, azetidinyl,

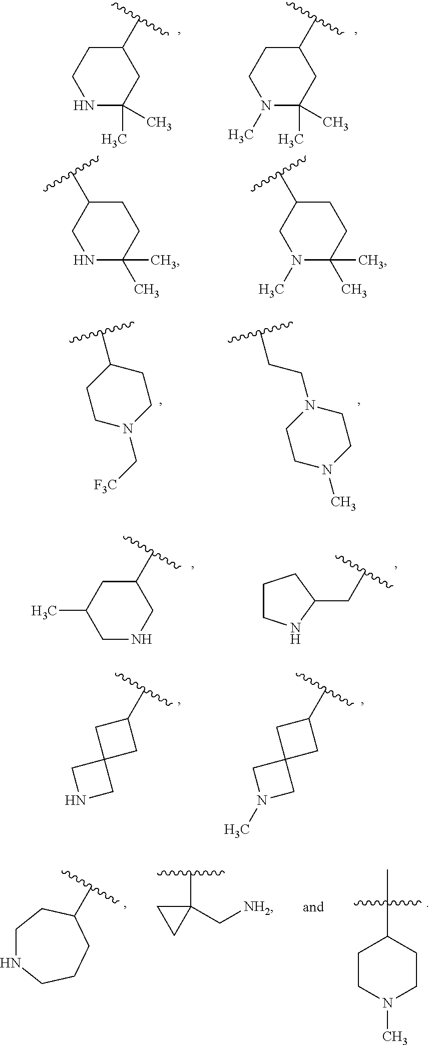

In some embodiments, R¹⁰ is selected from C₃₋₈cycloalkyl, aryl and 4-8 membered heterocyclyl, each of which may be optionally substituted with 1-3 groups selected from C₁₋₄alkyl, C₁₋₄alkyl-OH, C₁₋₄alkyl-NR′R″, wherein R′ and R″ are independently selected from H and C₁₋₄alkyl, haloC₁₋₄alkyl, halo, C₁₋₄alkoxy, and C₁₋₄alkoxyC₁₋₄alkyl.

In some embodiments, w is 0 or 1.

In some embodiments, w is selected from 0, 1 and 3.

In these embodiments, the compound may be provided as a compound of formula (IIF) or (IIG):

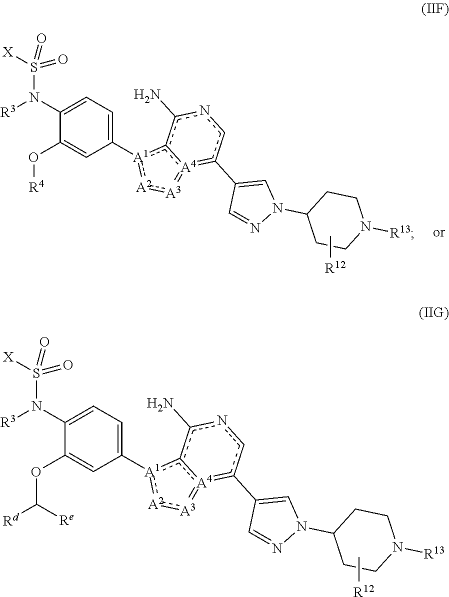

wherein A¹, A², A³, A⁴, R³, R⁴, R^{d} and R^{e} are as defined herein;
R¹² is one or more groups independently selected from H, C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆alkoxyaryl, halo, C₁₋₆alkylhalo, C₁₋₆alkoxyhalo and amino; and
R¹³ is selected from H, C₁₋₆alkyl and haloC₁₋₆alkyl.

In some embodiments, R¹² is one or more groups independently selected from H, C₁₋₆alkyl and C₃₋₈cycloalkyl. In some embodiments, R¹² is one or more groups independently selected from H, C₁₋₃alkyl and C₃₋₄cycloalkyl; preferably C₁alkyl and C₃cycloalkyl.

In some embodiments, R¹² substitutes the carbon alpha to the piperidyl nitrogen.

In some embodiments, R¹³ is selected from H, C₁₋₃alkyl and haloC₁₋₃alkyl; preferably C₁₋₃alkyl; more preferably C₁alkyl.

### J2

In some embodiments, J is J2. In these embodiments, the compound of formula (I) may be provided as a compound of formula (III)

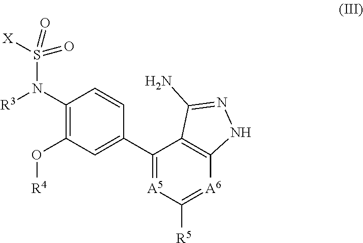

wherein X, R³, R⁴, R⁵, A⁵ and A⁶ are as defined for formula (I) and any embodiment thereof described herein.

In some embodiments, A⁵ is CH.

In some embodiments, A⁵ is N.

In some embodiments, A⁶ is N.

In some embodiments, A⁶ is CR².

In some embodiments, A⁵ is N and A⁶ is N.

In some embodiments, A⁵ is CH and A⁶ is N.

In some embodiments, A⁵ is N and A⁶ is CR².

In some embodiments, at least one of A⁵ and A⁶ is N.

In some embodiments, R⁵ is H.

In some embodiments, the compound of the invention is selected from any of compounds 1001 to 1031, 1037 and 1039 to 1145 described herein, preferably from any of compounds 1001 to 1031, 1037 and 1039 to 1137.

In some embodiments, the compound of the invention is selected from any of compounds 1001 to 1031 described herein, preferably from any of compounds 1001 to 1023 and 1029 to 1031, more preferably from any of compounds 1003, 1005, 1006, 1008 to 1012, 1014, 1015, 1017 and 1022.

In some embodiments, the compound comprises a radical of a compound selected from compounds 1-173 described herein.

In some embodiments, the compound of the invention comprises a radical of a compound selected from compounds 1-125 described herein.

In some embodiments, the compound of the invention comprises a radical of a compound selected from compounds 113, 116, 145, 154, 155, 170 and 171 described herein.

In some embodiments, MLKLi is a compound of formula (X) comprising a radical at:
i) R² when J is J1 so that R² and L are covalently bound; or
ii) R⁵ when J is J2 so that R⁵ and L are covalently bound.

In some embodiments, the compound of formula (X) may be provided as a compound of formula (XX):

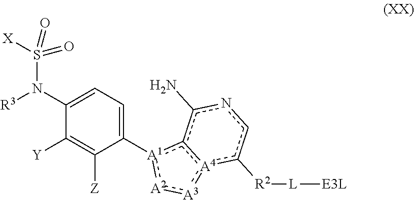

wherein A¹, A², A³, A⁴, n, R¹, R², R³, R⁴, R⁴, X, Y, Z, L and E3L are as defined herein.

In some embodiments, the compound of formula (X) may be provided as a compound of formula (XXI):

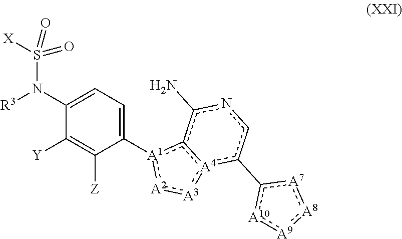

wherein A¹, A², A³, A⁴, n, R¹, R³, R⁴, R⁴, X, Y, Z, L and E3L are as defined herein;
A⁷, A⁸, A⁹ and A¹⁰ are independently selected from C(R¹²)_{q}, O, S, N, NR¹³, C(R¹²)_L-E3L, C-L-E3L and N-L-E3L;
one of A⁷, A⁸, A⁹ and A¹⁰ is selected from C(R¹²)-L-E3L, C-L-E3L and N-L-E3L; at least one of A⁷, A⁸, A⁹ and A¹⁰ is selected from C(R¹²)₂, 0, S, NR¹³, C(R¹²)-L-E3L;
each R¹² is independently selected from H and R¹⁴;
each R¹⁴ is independently selected from halo, C₁₋₆alkyl, C₁₋₆alkoxy, C₃₋₆cycloalkyl, —OC₁₋₆alkylC₁₋₆alkoxy, haloC₁₋₆alkyl, haloC₁₋₆alkoxy, nitrile, amido, C₁₋₆alkylamido, (C₁₋₆alkyl)₂amido, haloC₁₋₆alkylamido, (haloC₁₋₆alkyl)₂amido, acyl, C₁₋₆alkylacyl, haloC₁₋₆ alkylacyl, arylacyl, heterocyclylacyl, cycloalkylacyl, heterocyclyl, haloC₁₋₆alkoxy, C₃₋₁₀cycloalkyl, C₁₋₆alkylC₃₋₁₀cycloalkyl, C₁₋₆alkoxyC₃₋₁₀cycloalkyl, haloC₁₋₆alkylC₃₋₁₀cycloalkyl, haloC₁₋₆alkoxyC₃₋₁₀cycloalkyl, C₁₋₆alkylheterocyclyl, C₁₋₆alkoxyheterocyclyl, haloC₁₋₆ alkylheterocyclyl, haloC₁₋₆alkoxyheterocyclyl, C₁₋₆alkylC₁₋₆alkoxy, and —COOH;
each R¹³ is independently selected from H, C₁₋₆alkyl, haloC₁₋₄alkyl, C₁₋₆alkylacyl and haloC₁₋₆alkylacyl;
or when two adjacent groups selected from A⁷, A⁸, A⁹ and A¹⁰ are selected from CR¹², C(R¹²)-L-E3L and NR¹³, two R¹², two R¹³ or one R¹² and one R¹³ may together form an optionally substituted 5-10 membered ring selected from cycloalkyl, aryl and heterocyclyl;
q is 1 or 2.

In some embodiments, A⁹ is N, A⁸ is N-L-E3L, A⁷ is CR¹² and A¹⁰ is CR¹².

In some embodiments, the compound of formula (X) may be provided as a compound of formula (XXII):

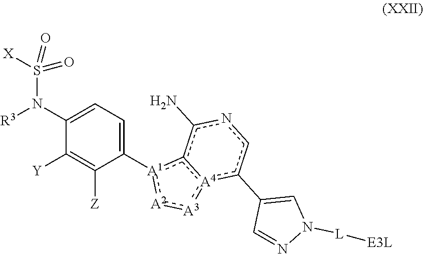

wherein A¹, A², A³, A⁴, n, R¹, R³, R⁴, R⁴, X, Y, Z, L and E3L are as defined herein.

In some embodiments, the compound of formula (X) may be provided as a compound of formula (XXIII):

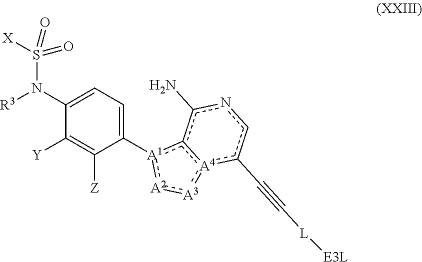

wherein A¹, A², A³, A⁴, n, R¹, R³, R⁴, R⁴, X, Y, Z, L and E3L are as defined herein.

In some embodiments, the compound of formula (X) may be provided as a compound of formula (XXIV):

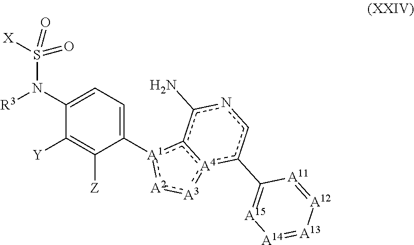

wherein A¹, A², A³, A⁴, n, R¹, R³, R⁴, R⁴, X, Y, Z, L and E3L are as defined herein;
wherein A¹¹-A¹⁵ are independently selected from N, CR¹² and C-L-E3L, one of A¹¹-A¹⁵ is C-L-E3L wherein not more than 2 of A¹, A¹², A¹³, A¹⁴ and A¹⁵ are N;
each R¹² is independently selected from H and R¹⁴;
each R¹⁴ is independently selected from halo, C₁₋₆alkyl, C₁₋₆alkoxy, C₃₋₆cycloalkyl, —OC₁₋₆alkylC₁₋₆alkoxy, haloC₁₋₆alkyl, haloC₁₋₆alkoxy, nitrile, amido, C₁₋₆alkylamido, (C₁₋₆alkyl)₂amido, haloC₁₋₆alkylamido, (haloC₁₋₆alkyl)₂amido, acyl, C₁₋₆alkylacyl, haloC₁₋₆ alkylacyl, arylacyl, heterocyclylacyl, cycloalkylacyl, heterocyclyl, haloC₁₋₆alkoxy, C₃₋₁₀cycloalkyl, C₁₋₆alkylC₃₋₁₀cycloalkyl, C₁₋₆alkoxyC₃₋₁₀cycloalkyl, haloC₁₋₆ alkylC₃-10cycloalkyl, haloC₁₋₆alkoxyC₃-10cycloalkyl, C₁₋₆alkylheterocyclyl, C₁₋₆alkoxyheterocyclyl, haloC₁₋₆alkylheterocyclyl, haloC₁₋₆alkoxyheterocyclyl, C₁₋₆alkylC₁₋₆alkoxy, and —COOH;
or when two adjacent groups selected from A¹¹, A¹², A¹³, A¹⁴, A¹⁵, are CR¹², two R¹² may together form an optionally substituted 5-10 membered ring selected from cycloalkyl, aryl and heterocyclyl.

A¹¹-A¹⁵ may be defined as any one of embodiments 1-4:

| Embodiment | | | | | |
|---|---|---|---|---|---|
| No. | A¹¹ | A¹² | A¹³ | A¹⁴ | A¹⁵ |
| 1 | CR¹² | C-L-E3L | N | CR¹² | CR¹² |
| 2 | N | CR¹² | C-L-E3L | CR¹² | CR¹² |
| 3 | N | CR¹² | C-L-E3L | N | CR¹² |
| 4 | N | C-L-E3L | N | CR¹² | CR¹² |

In some embodiments, the compound of formula (X) may be provided as a compound of formula (XXa):

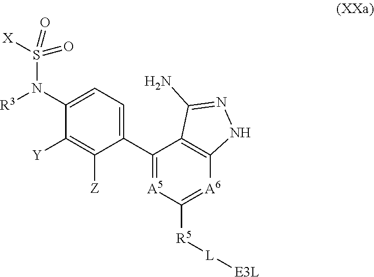

wherein A⁵, A⁶, R¹, R², R³, R⁴, R⁴, R⁵, X, Y, Z, L and E3L are as defined herein.

In some embodiments, the compound of formula (X) may be provided as a compound of formula (XXIa):

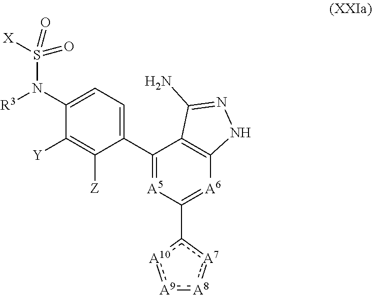

wherein A⁵, A⁶, R¹, R², R³, R⁴, R⁴, X, Y, Z, L and E3L are as defined herein;
A⁷, A⁸, A⁹ and A¹⁰ are independently selected from C(R¹²)_{q}, O, S, N, NR¹³, C(R¹²)_L-E3L, C-L-E3L and N-L-E3L;
one of A⁷, A⁸, A⁹ and A¹⁰ is selected from C(R¹²)-L-E3L, C-L-E3L and N-L-E3L;
at least one of A⁷, A⁸, A⁹ and A¹⁰ is selected from C(R¹²)₂, 0, S, NR¹³, C(R¹²)-L-E3L;
each R¹² is independently selected from H and R¹⁴;
each R¹⁴ is independently selected from halo, C₁₋₆alkyl, C₁₋₆alkoxy, C₃₋₆cycloalkyl, —OC₁₋₆alkylC₁₋₆alkoxy, haloC₁₋₆alkyl, haloC₁₋₆alkoxy, nitrile, amido, C₁₋₆alkylamido, (C₁₋₆alkyl)₂amido, haloC₁₋₆alkylamido, (haloC₁₋₆alkyl)₂amido, acyl, C₁₋₆alkylacyl, haloC₁₋₆ alkylacyl, arylacyl, heterocyclylacyl, cycloalkylacyl, heterocyclyl, haloC₁₋₆alkoxy, C₃₋₁₀cycloalkyl, C₁₋₆alkylC₃₋₁₀cycloalkyl, C₁₋₆alkoxyC₃₋₁₀cycloalkyl, haloC₁₋₆alkylC₃₋₁₀cycloalkyl, haloC₁₋₆alkoxyC₃₋₁₀cycloalkyl, C₁₋₆alkylheterocyclyl, C₁₋₆alkoxyheterocyclyl, haloC₁₋₆ alkylheterocyclyl, haloC₁₋₆alkoxyheterocyclyl, C₁₋₆alkylC₁₋₆alkoxy, and —COOH;
each R¹³ is independently selected from H, C₁₋₆alkyl, haloC₁₋₄alkyl, C₁₋₆alkylacyl and haloC₁₋₆alkylacyl;
or when two adjacent groups selected from A⁷, A⁸, A⁹ and A¹⁰ are selected from CR¹², C(R¹²)-L-E3L and NR¹³, two R¹², two R¹³ or one R¹² and one R¹³ may together form an optionally substituted 5-10 membered ring selected from cycloalkyl, aryl and heterocyclyl;
q is 1 or 2.

In some embodiments, A⁹ is N, A⁸ is N-L-E3L, A⁷ is CR¹² and A¹⁰ is CR¹².

In some embodiments, the compound of formula (X) may be provided as a compound of formula (XXIla):

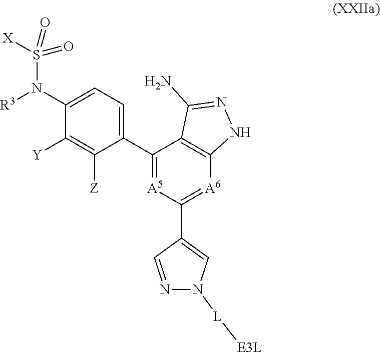

wherein A⁵, A⁶, R¹, R², R³, R⁴, R⁴, X, Y, Z, L and E3L are as defined herein.

In some embodiments, the compound of formula (X) may be provided as a compound of formula (XXIIIa):

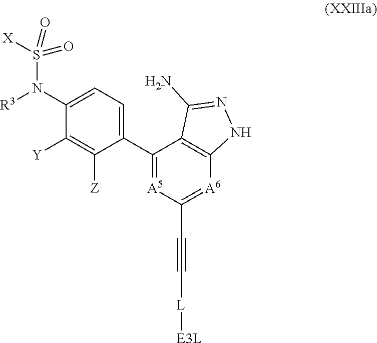

wherein A⁵, A⁶, R¹, R², R³, R⁴, R⁴, X, Y, Z, L and E3L are as defined herein.

In some embodiments, the compound of formula (X) may be provided as a compound of formula (XXIVa):

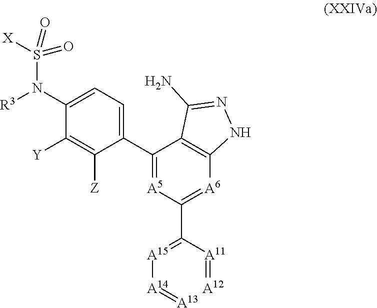

wherein A⁵, A⁶, R¹, R², R³, R⁴, R⁴, X, Y, Z, L and E3L are as defined herein;
wherein A¹¹-A¹⁵ are independently selected from N, CR¹² and C-L-E3L, one of A¹¹-A¹⁵ is C-L-E3L
wherein not more than 2 of A¹, A¹², A¹³, A¹⁴ and A¹⁵ are N;
each R¹² is independently selected from H and R¹⁴;
each R¹⁴ is independently selected from halo, C₁₋₆alkyl, C₁₋₆alkoxy, C₃₋₆cycloalkyl, —OC₁₋₆alkylC₁₋₆alkoxy, haloC₁₋₆alkyl, haloC₁₋₆alkoxy, nitrile, amido, C₁₋₆alkylamido, (C₁₋₆alkyl)₂amido, haloC₁₋₆alkylamido, (haloC₁₋₆alkyl)₂amido, acyl, C₁₋₆alkylacyl, haloC₁₋₆ alkylacyl, arylacyl, heterocyclylacyl, cycloalkylacyl, heterocyclyl, haloC₁₋₆alkoxy, C₃₋₁₀cycloalkyl, C₁₋₆alkylC₃₋₁₀cycloalkyl, C₁₋₆alkoxyC₃₋₁₀cycloalkyl, haloC₁₋₆ alkylC₃-10cycloalkyl, haloC₁₋₆alkoxyC₃-10cycloalkyl, C₁₋₆alkylheterocyclyl, C₁₋₆alkoxyheterocyclyl, haloC₁₋₆alkylheterocyclyl, haloC₁₋₆alkoxyheterocyclyl, C₁₋₆alkylC₁₋₆alkoxy, and —COOH;
or when two adjacent groups selected from A¹¹, A¹², A¹³, A¹⁴, A¹⁵, are CR¹², two R¹² may together form an optionally substituted 5-10 membered ring selected from cycloalkyl, aryl and heterocyclyl.

A¹¹-A¹⁵ may be defined as any one of embodiments 1-4:

| Embodiment | | | | | |
|---|---|---|---|---|---|
| No. | A¹¹ | A¹² | A¹³ | A¹⁴ | A¹⁵ |
| 1 | CR¹² | C-L-E3L | N | CR¹² | CR¹² |
| 2 | N | CR¹² | C-L-E3L | CR¹² | CR¹² |
| 3 | N | CR¹² | C-L-E3L | N | CR¹² |
| 4 | N | C-L-E3L | N | CR¹² | CR¹² |

### E3L

In the compounds of formula (X) E3L denotes an E3 ligase binding moiety. Any suitable E3 ligase binding moiety may be included in the compounds of the invention. Suitable E3 ligase binding moieties include different suitable linker attachment points and/or different suitable stereochemistries of E3 ligase binding moieties. E3 ligase binding moieties have been reviewed in Bricelj et al, Front. Chem., 2021, 9, 707317, Schapira et al, Nat Rev Drug Discovery, 2019, 18, 949, Bricelj, A. et al., Front. Chem. 2021, 9, 707317, Maple, H. J. et al., Med. Chem. Commun., 2019, 10, 1755, and Ishida, T. and Ciulli, A. SLAS Discovery, 2021, 26(4), 484, the entire contents of each of which is incorporated by reference. The skilled person would appreciate that a suitable E3 ligase binding moiety includes the E3 ligase binding structures depicted in Bricelj et al, Front. Chem., 2021, 9, 707317, Schapira et al, Nat Rev Drug Discovery, 2019, 18, 949, Bricelj, A. et al., Front. Chem. 2021, 9, 707317, Maple, H. J. et al., Med. Chem. Commun., 2019, 10, 1755, and Ishida, T. and Ciulli, A. SLAS Discovery, 2021, 26(4), 484; as well as E3 ligase binding structures that features different suitable linker attachment points and/or different suitable stereochemistries.

The human genome includes more than 600 E3 ligases (or E3 ubiquitin ligases). E3 ligases are involved in the protein ubiquitination cascade, whereby one or more molecules of ubiquitin are linked to a substrate protein, marking those proteins for degradation via the ubiquitin-proteasome pathway. The E3 ligases are categorised into 3 broad classes: Really Interesting New Gene (RING), Homologous to E6AP C-terminus (HECT) and RING-between-RING (RBR). Of these, RING E3 ligases are the most common. Accordingly, the E3 ligase binding moiety may bind a RING, HECT or RBR E3 ligase, with E3L typically representing a RING E3 ligase binding moiety. Examples include, without limitation, MDM2 (mouse double minute 2 homologue) and cellular IAP (inhibitors of apoptosis).

E3L may also be a moiety capable of binding any of these E3 ligases. Binders of the following E3 ligases have been described: RING-type zinc-finger protein 114 (RNF114), damage-specific DNA binding protein 1 (DDB1)-CUL4 associated factor 16 (DCAF16), Kelchlike ECH-associated protein 1 (KEAP1), cereblon (CRBL or CRBN) and von Hippel-Landau (VHL) tumor suppressor protein. Preferably when E3L is a moiety capable of binding any of these E3 ligases, it is a moiety capable of binding CRBL and/or VHL.

In some embodiments, the E3 ligase binding moiety is selected from:

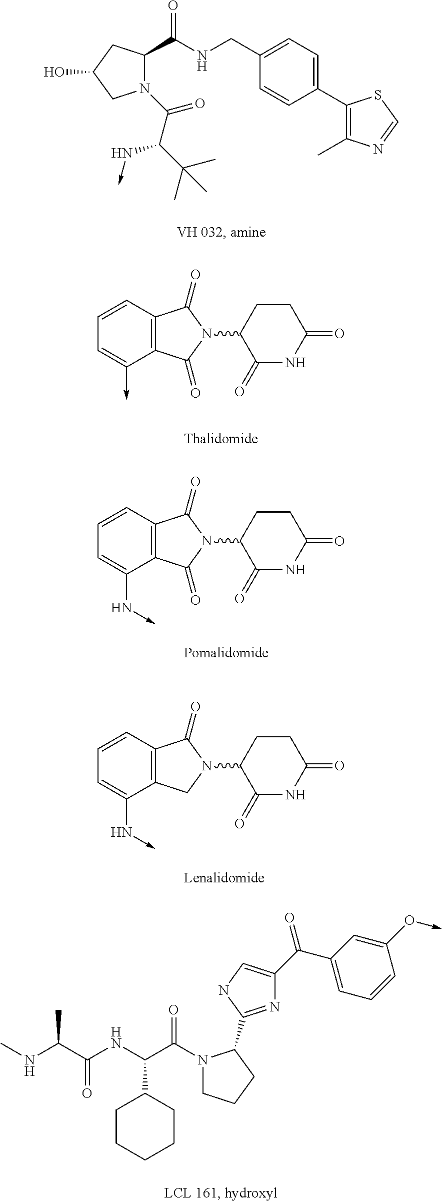

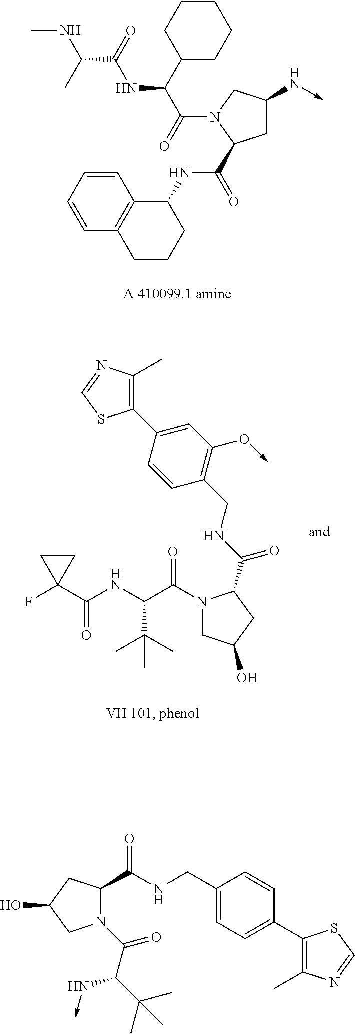

wherein the arrow denotes the covalent bond to L
or an E3 ligase binding derivative thereof.

In some embodiments, the E3 ligase binding moiety is selected from:

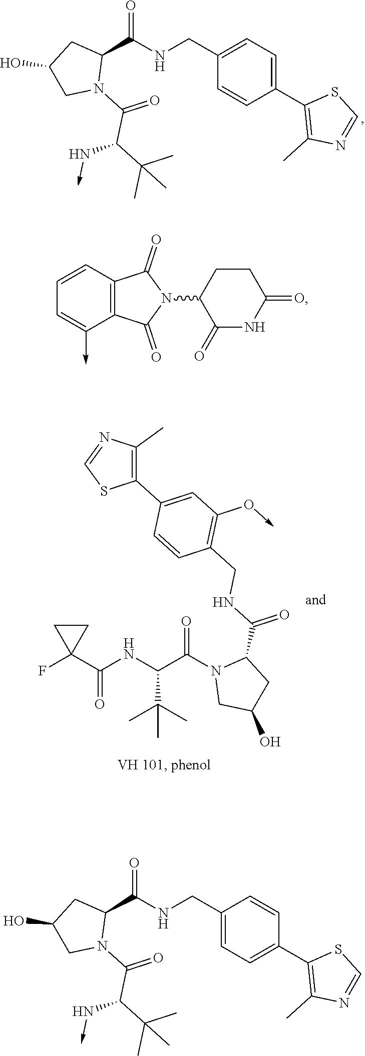

wherein the arrow denotes the covalent bond to L
or an E3 ligase binding derivative thereof.

In some embodiments, the E3 ligase binding moiety is selected from:

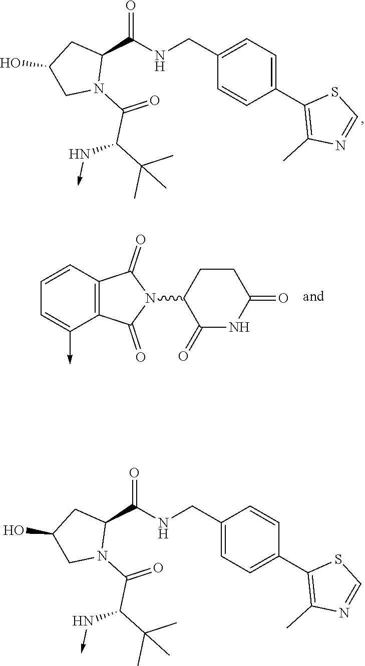

wherein the arrow denotes the covalent bond to L
or an E3 ligase binding derivative thereof.

In some embodiments, the E3 ligase binding moiety is:

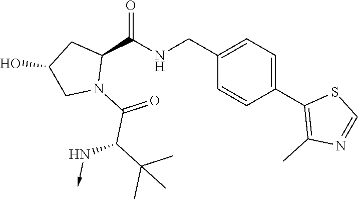

wherein the arrow denotes the covalent bond to L
or an E3 ligase binding derivative thereof.

In some embodiments, the E3 ligase binding moiety is:

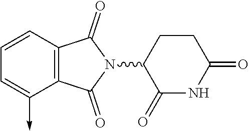

wherein the arrow denotes the covalent bond to L
or an E3 ligase binding derivative thereof.

In some embodiments, the E3 ligase binding moiety is selected from a radical of:

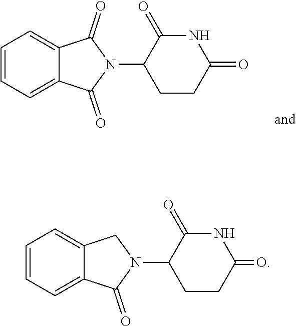

In some embodiments, the E3 ligase binding moiety is selected from:

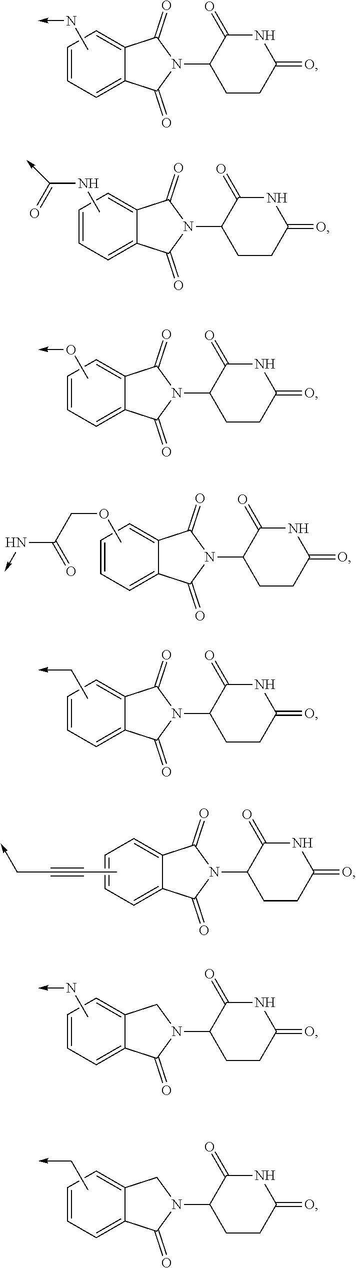

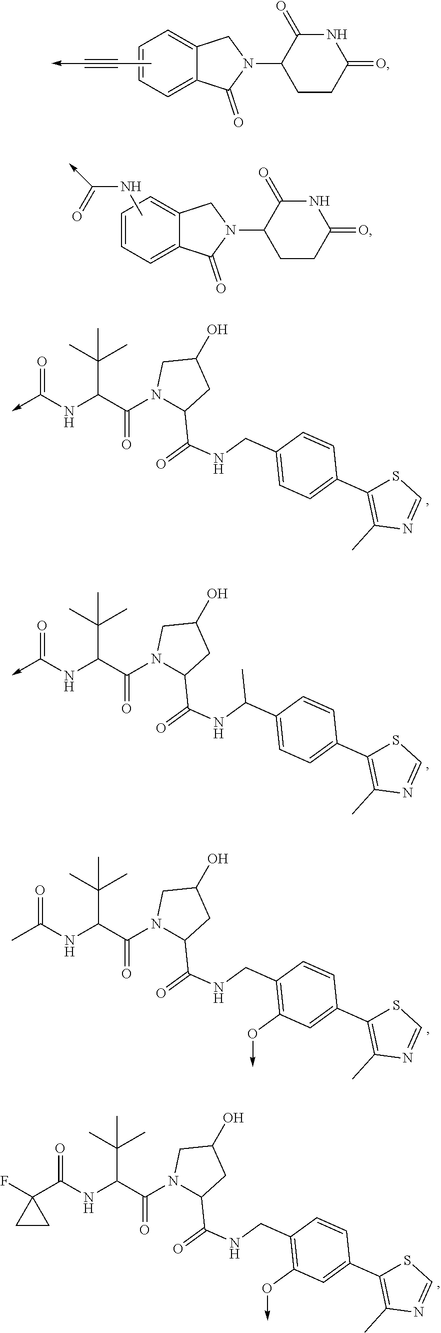

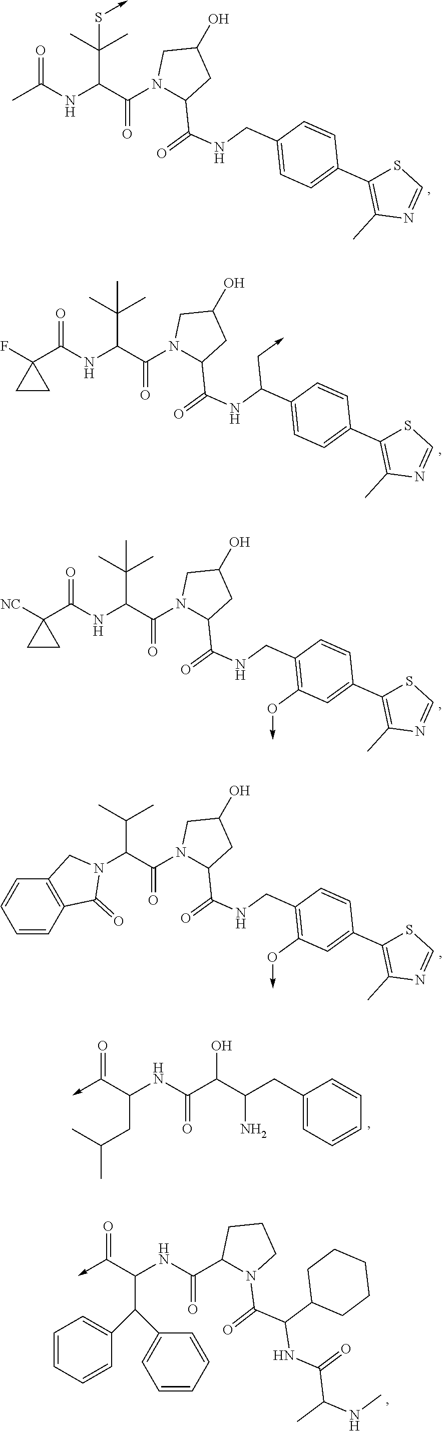

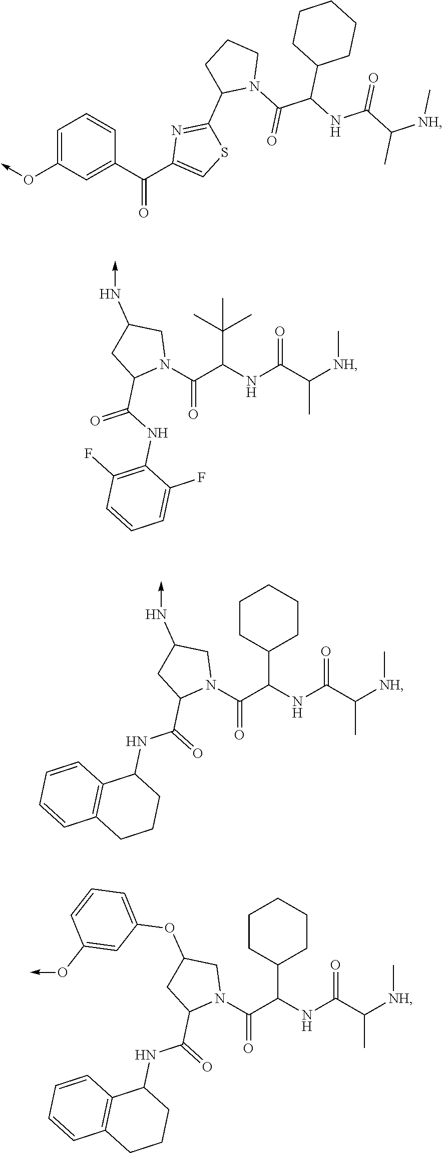

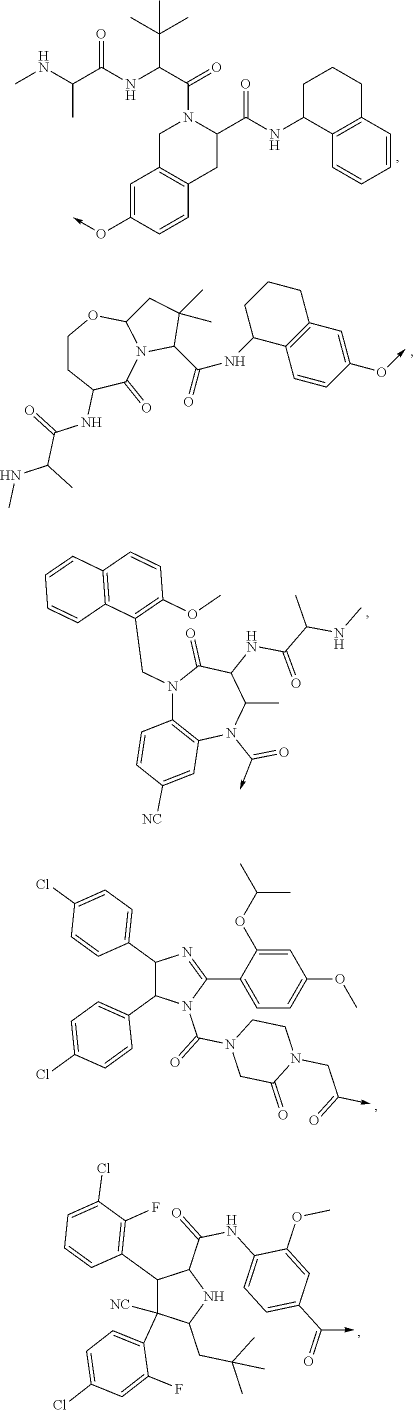

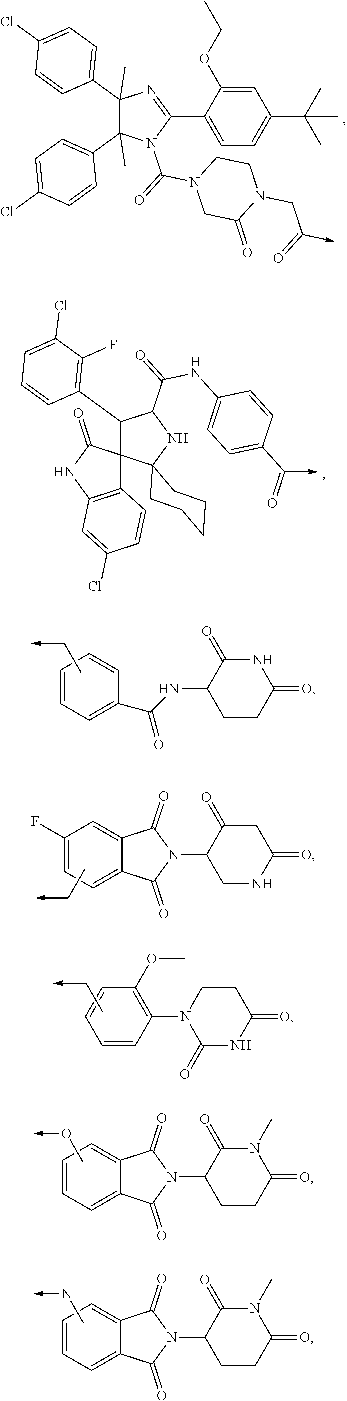

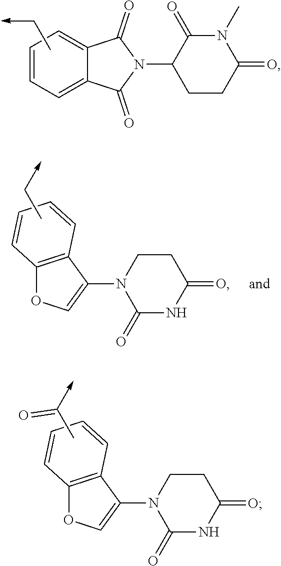

wherein the arrow denotes the covalent bond to L,
the portion of L not depicted in the structure, or
an E3 ligase binding derivative thereof.

In some embodiments, the E3 ligase binding moiety is selected from:

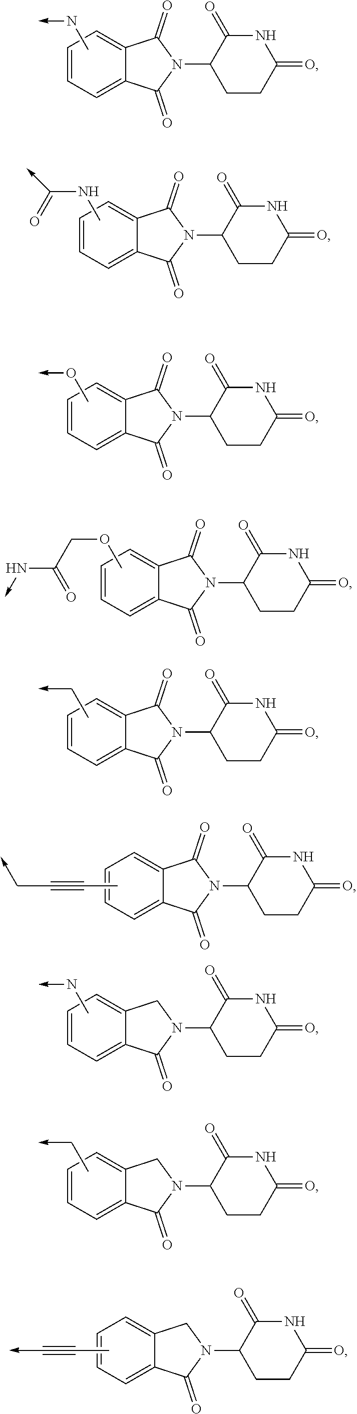

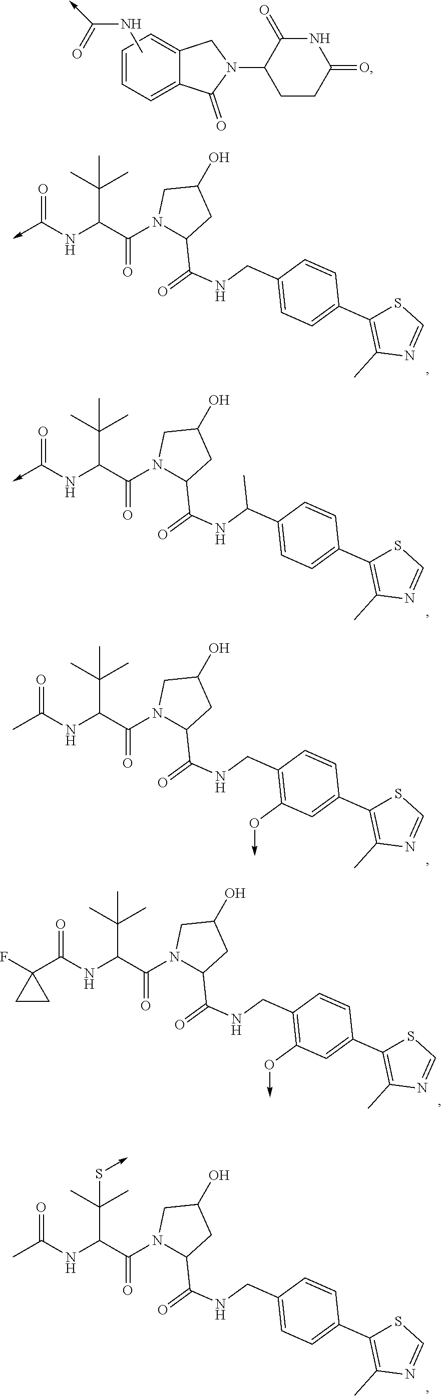

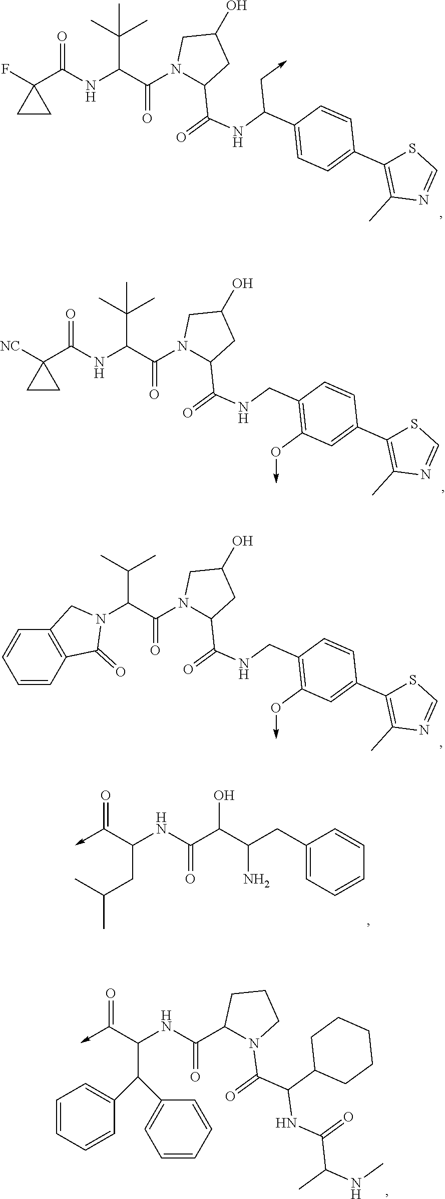

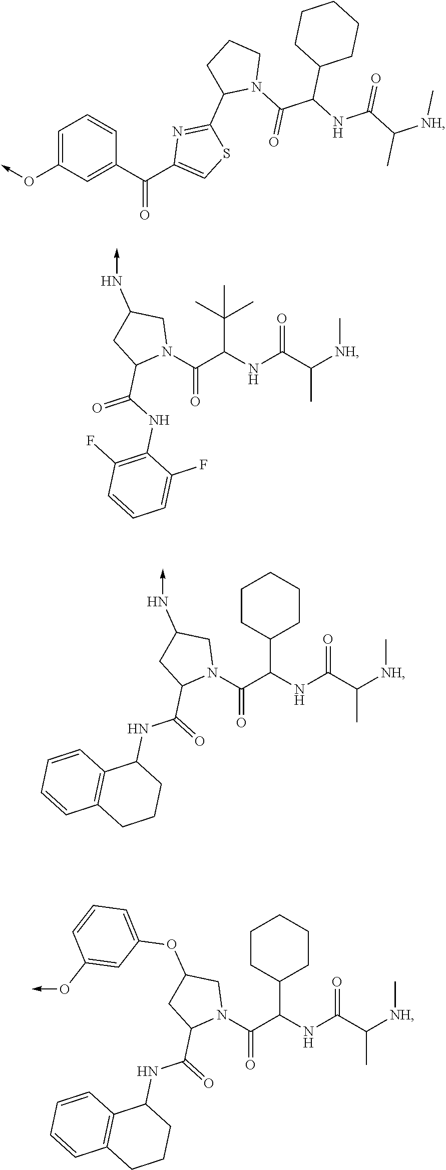

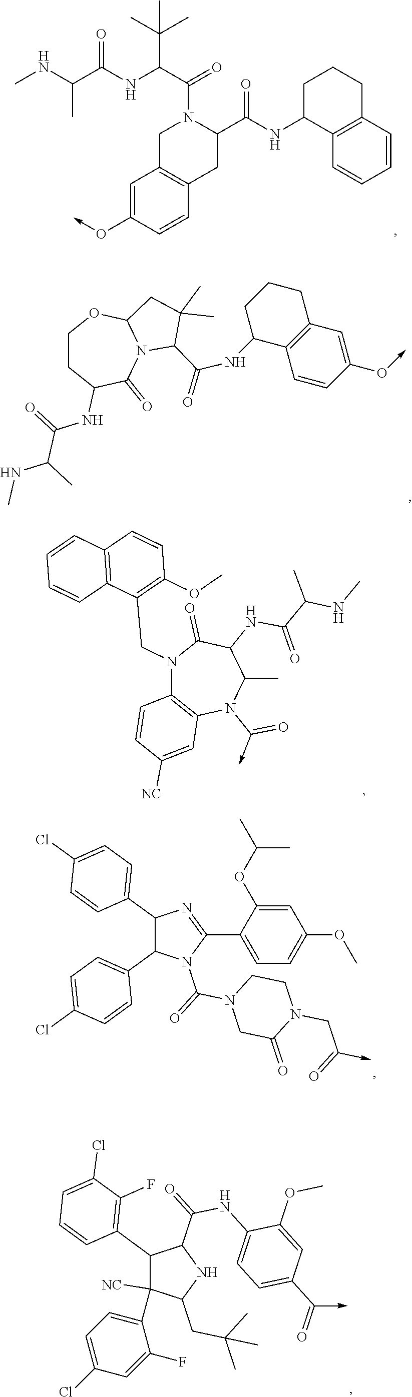

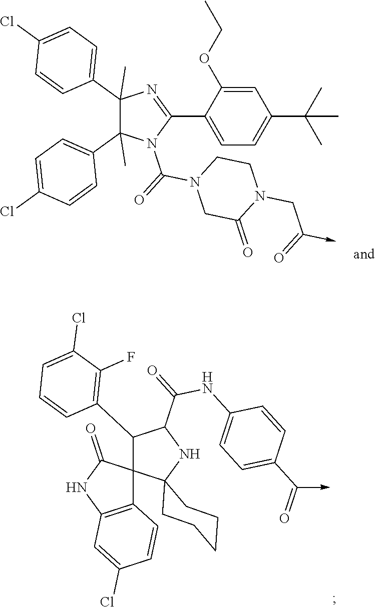

wherein the arrow denotes the covalent bond to L,
the portion of L not depicted in the structure, or
an E3 ligase binding derivative thereof.

In some embodiments, the E3 ligase binding moiety is selected from:

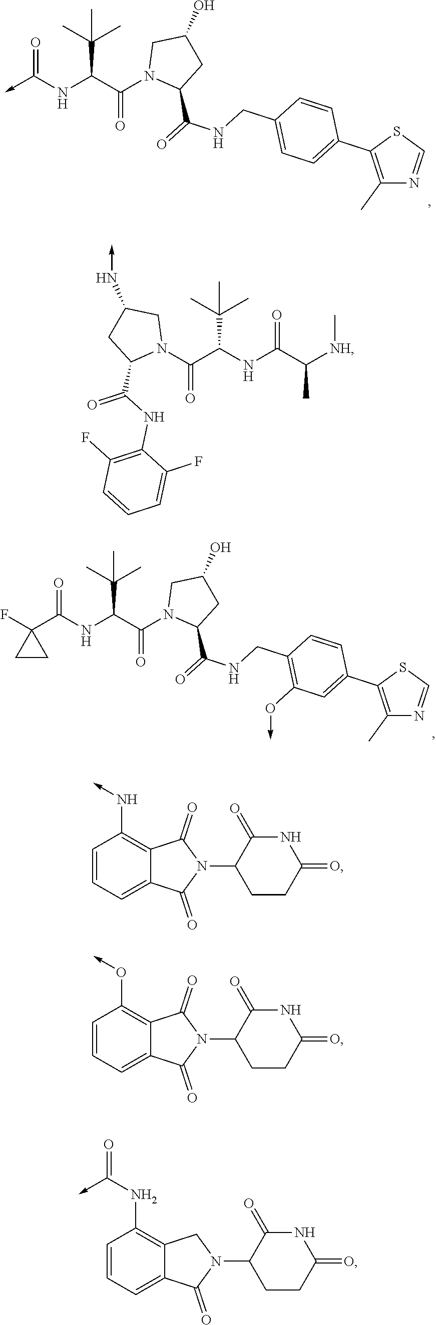

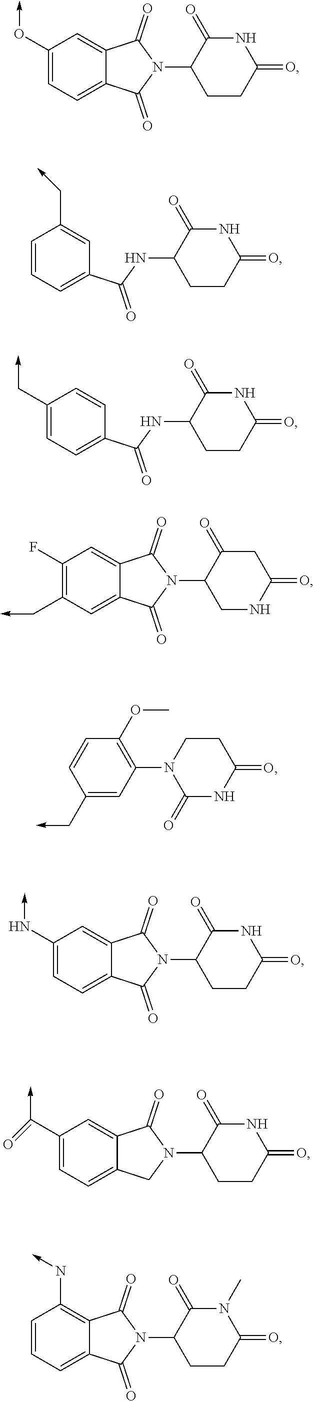

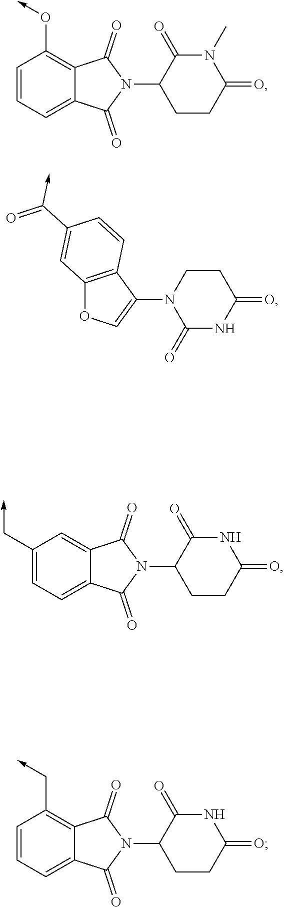

wherein the arrow denotes the covalent bond to L,
the portion of L not depicted in the structure, or
an E3 ligase binding derivative thereof.

In some embodiments, the E3 ligase binding moiety is selected from:

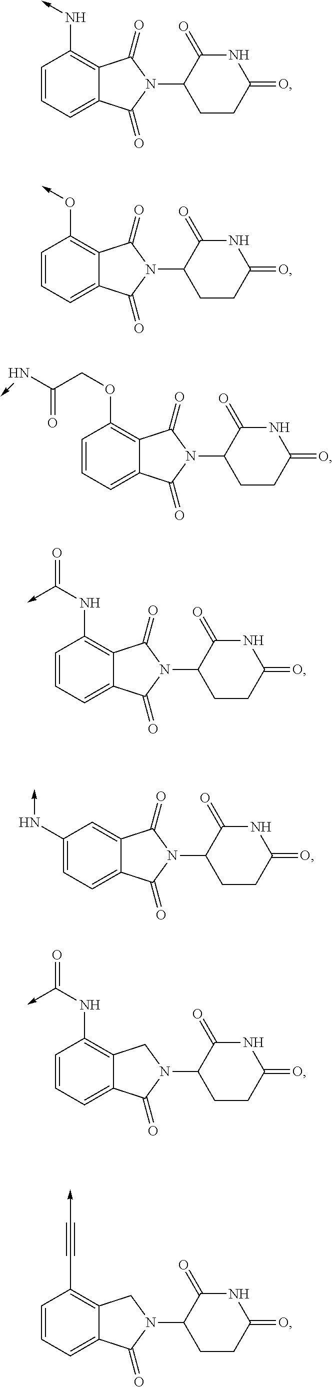

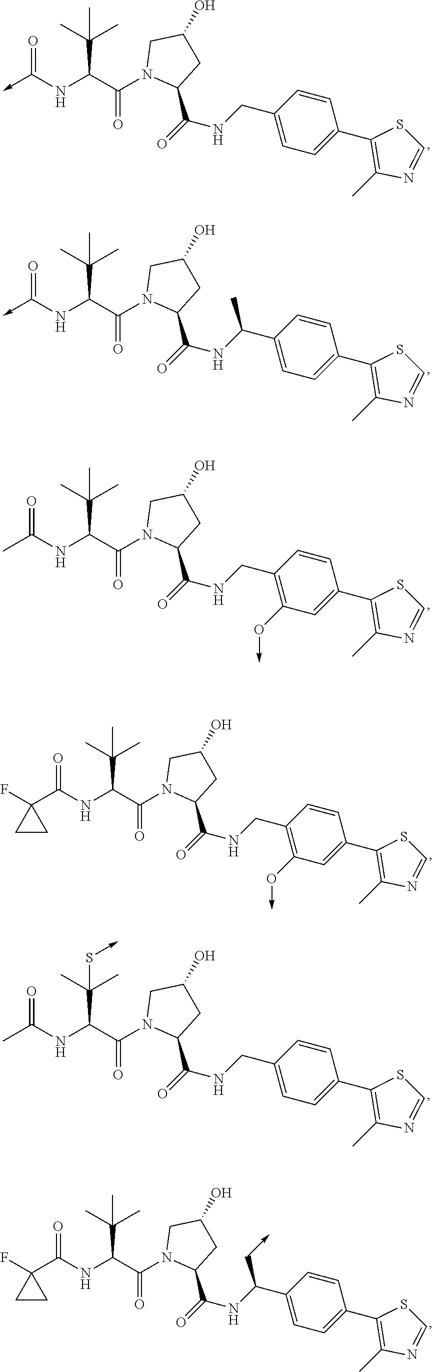

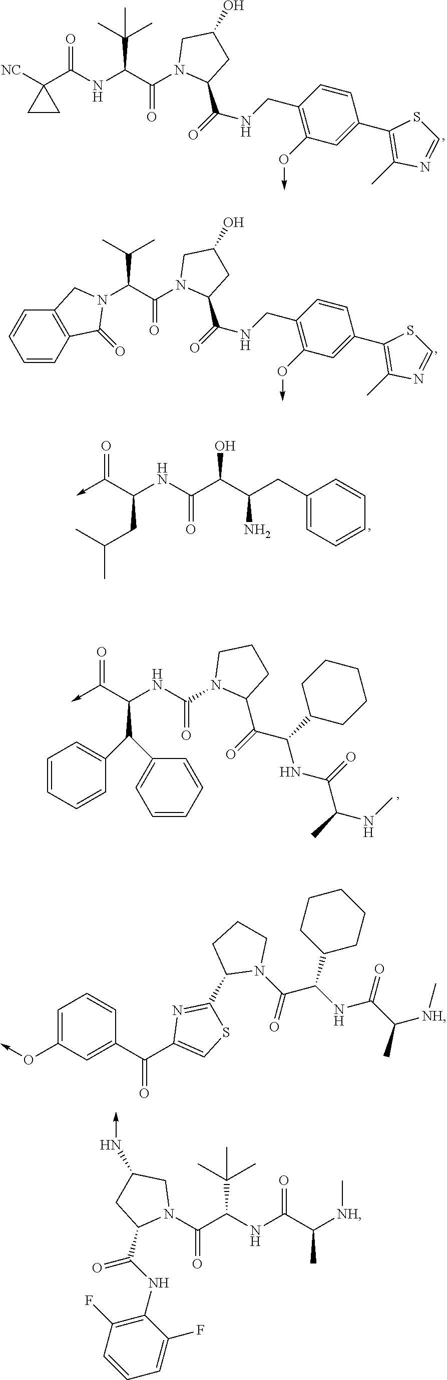

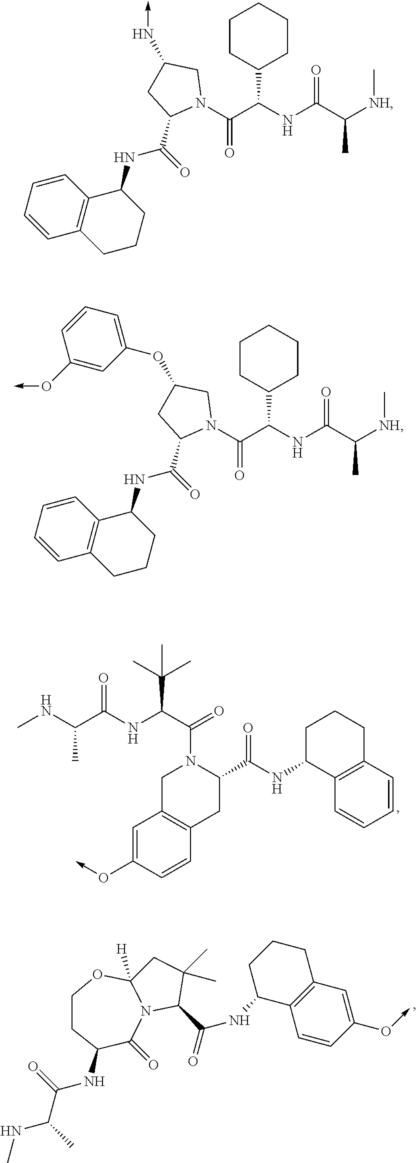

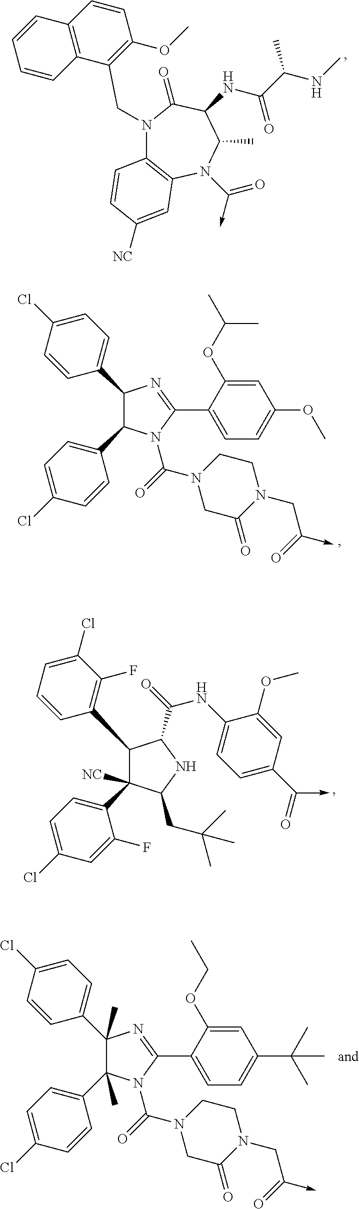

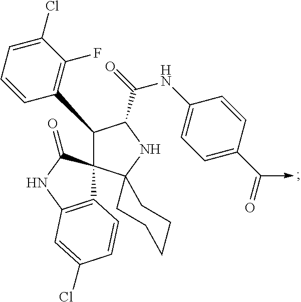

wherein the arrow denotes the covalent bond to L,
the portion of L not depicted in the structure, or
an E3 ligase binding derivative thereof.

In some embodiments, the E3 ligase binding moiety is selected from:

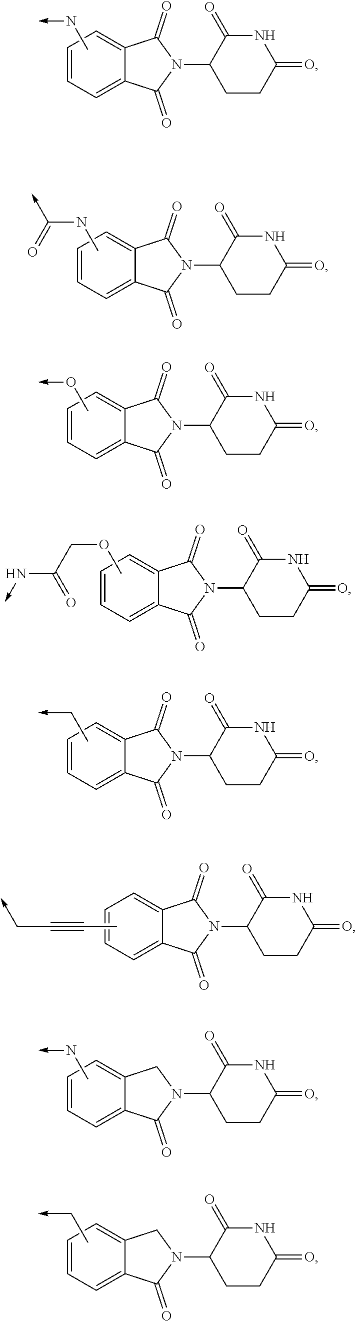

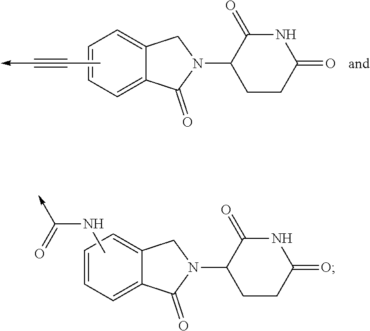

wherein the arrow denotes the covalent bond to L,
the portion of L not depicted in the structure, or
an E3 ligase binding derivative thereof.

In some embodiments, the E3 ligase binding moiety is selected from:

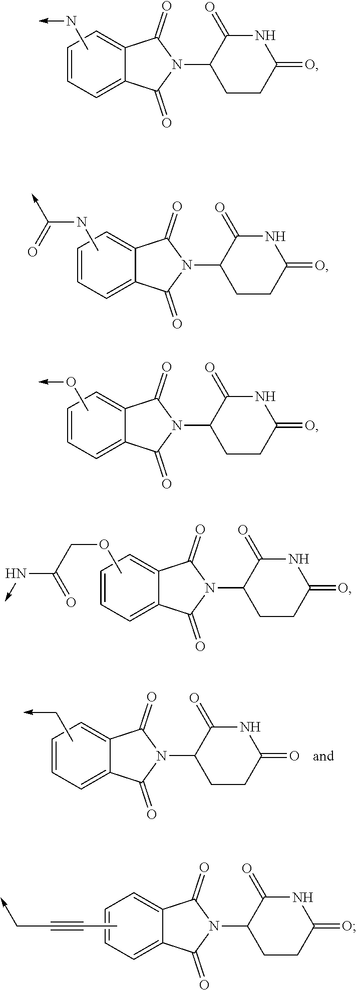

wherein the arrow denotes the covalent bond to L,
the portion of L not depicted in the structure, or
an E3 ligase binding derivative thereof.

In some embodiments, the E3 ligase binding moiety is selected from:

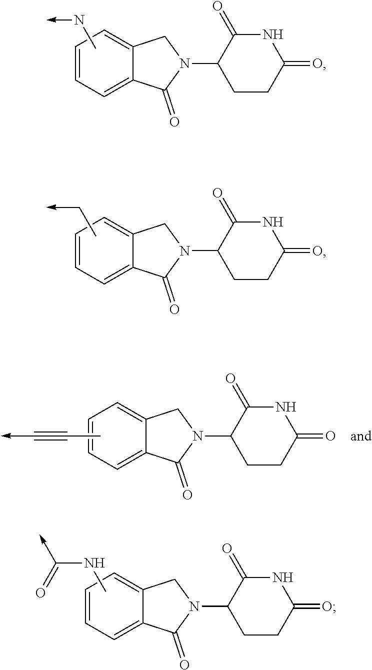

wherein the arrow denotes the covalent bond to L,
the portion of L not depicted in the structure, or
an E3 ligase binding derivative thereof.

In some embodiments, the E3 ligase binding moiety is selected from:

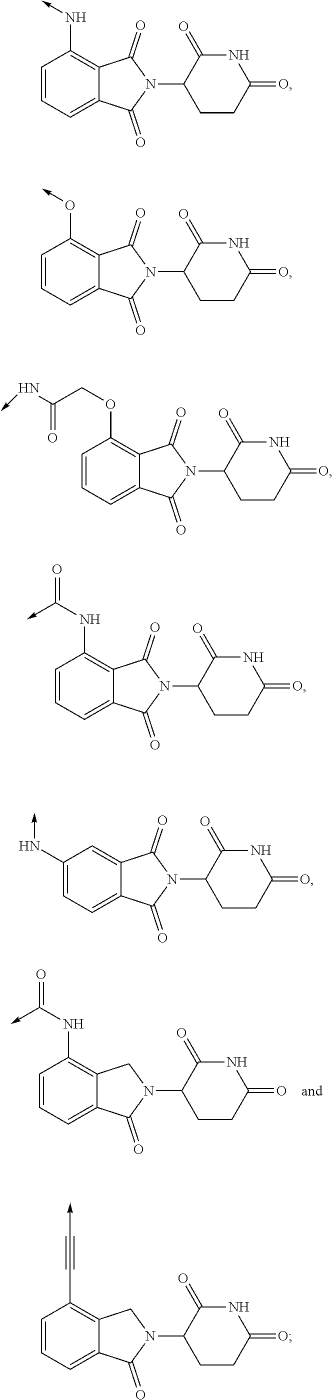

wherein the arrow denotes the covalent bond to L,
the portion of L not depicted in the structure, or
an E3 ligase binding derivative thereof.

In some embodiments, the E3 ligase binding moiety is selected from:

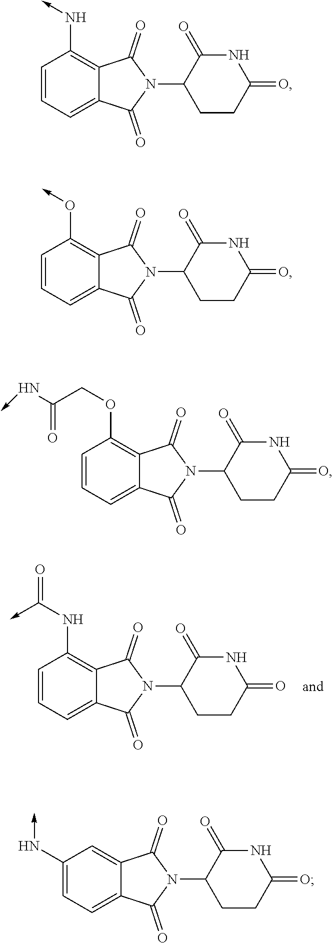

wherein the arrow denotes the covalent bond to L,
the portion of L not depicted in the structure, or
an E3 ligase binding derivative thereof.

In some embodiments, the E3 ligase binding moiety is selected from:

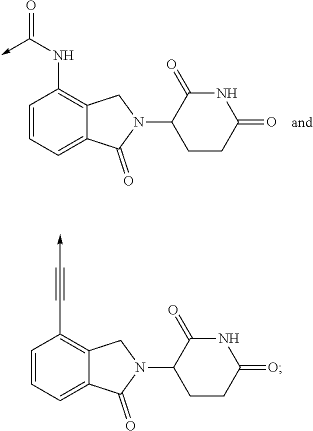

wherein the arrow denotes the covalent bond to L,
the portion of L not depicted in the structure, or
an E3 ligase binding derivative thereof.

In some embodiments, the E3 ligase binding moiety is selected from:

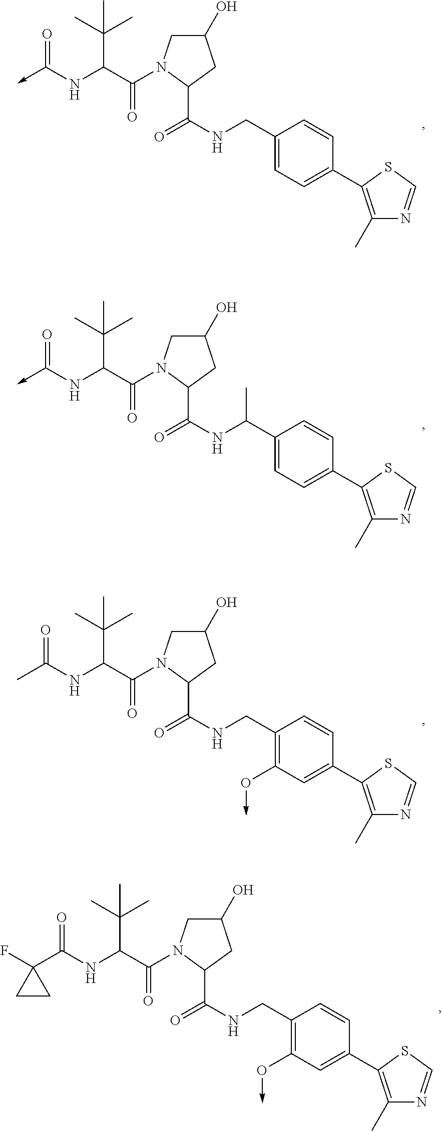

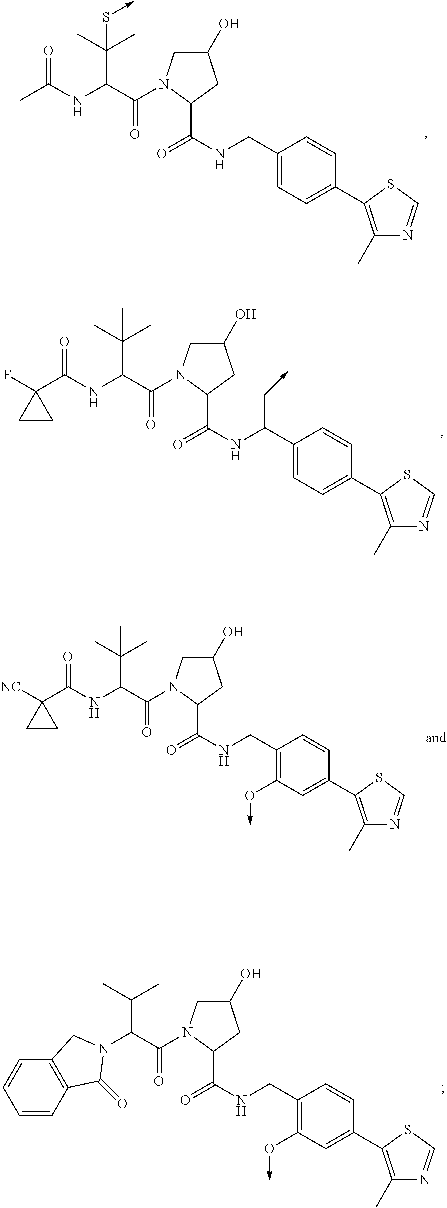

wherein the arrow denotes the covalent bond to L
or an E3 ligase binding derivative thereof.

In some embodiments, the E3 ligase binding moiety is selected from:

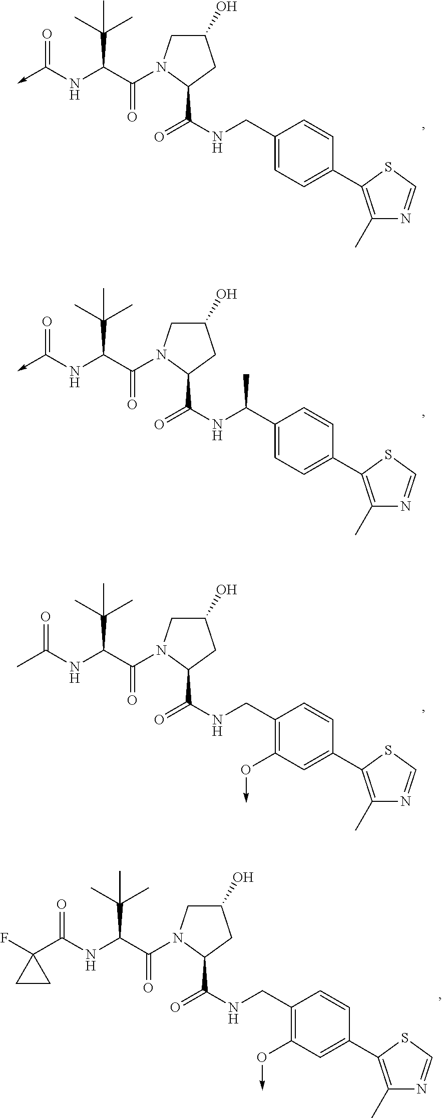

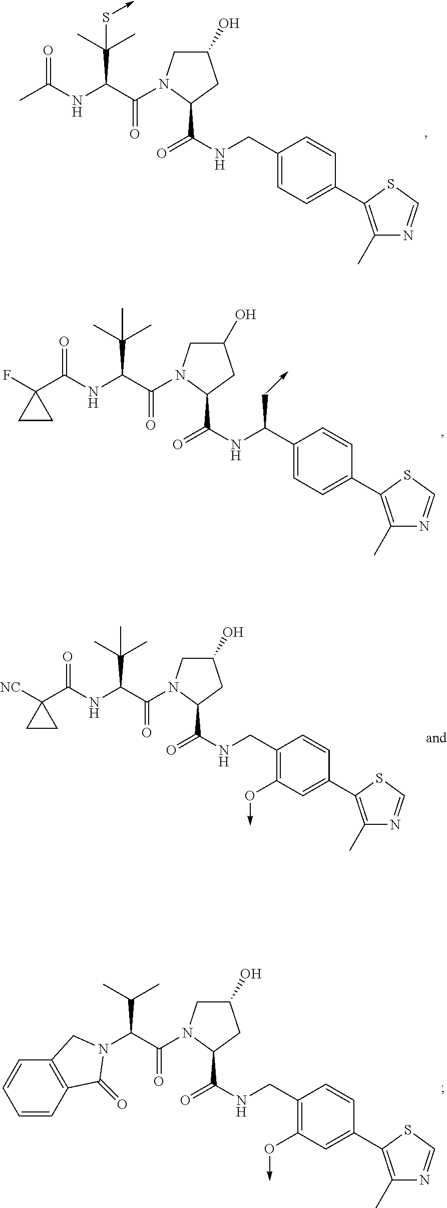

wherein the arrow denotes the covalent bond to L
or an E3 ligase binding derivative thereof.

In some embodiments, the E3 ligase binding moiety is selected from:

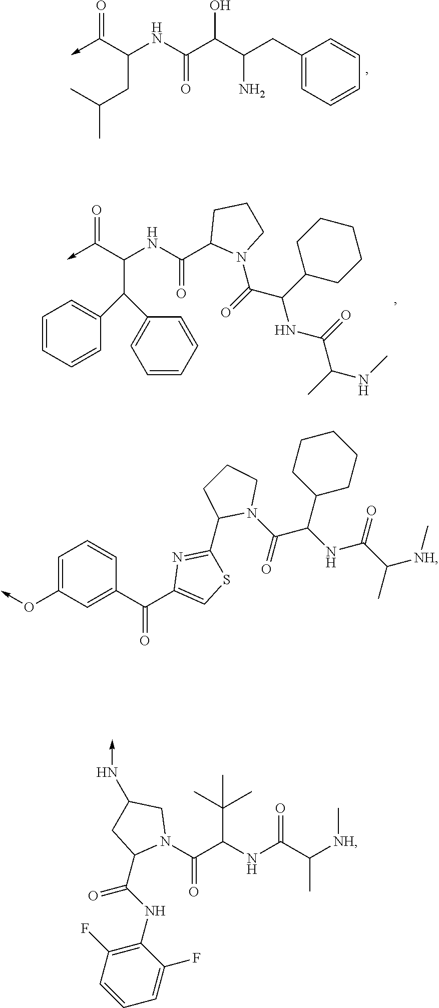

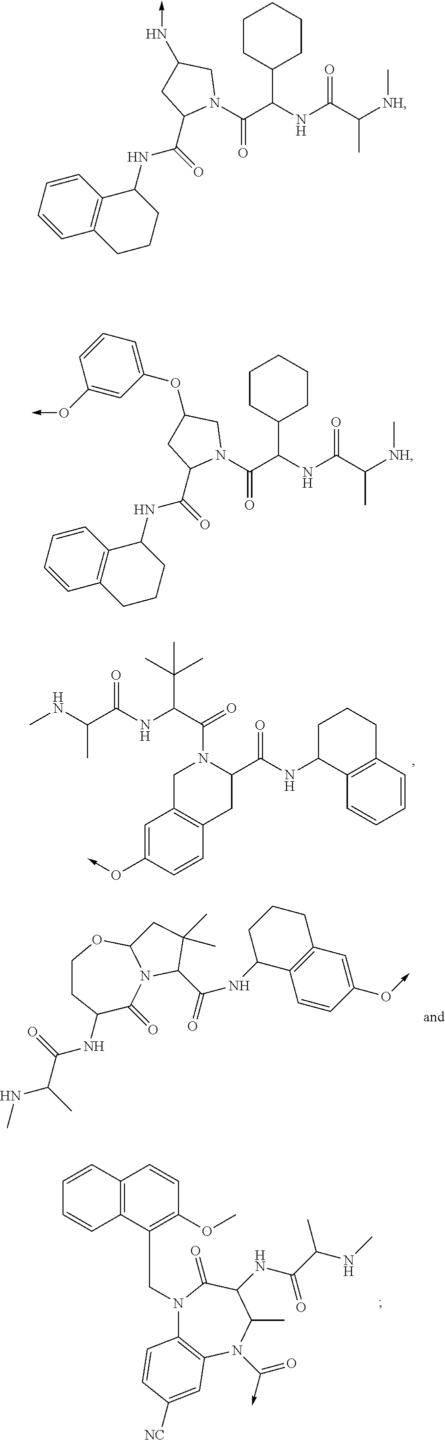

wherein the arrow denotes the covalent bond to L
or an E3 ligase binding derivative thereof.

In some embodiments, the E3 ligase binding moiety is selected from:

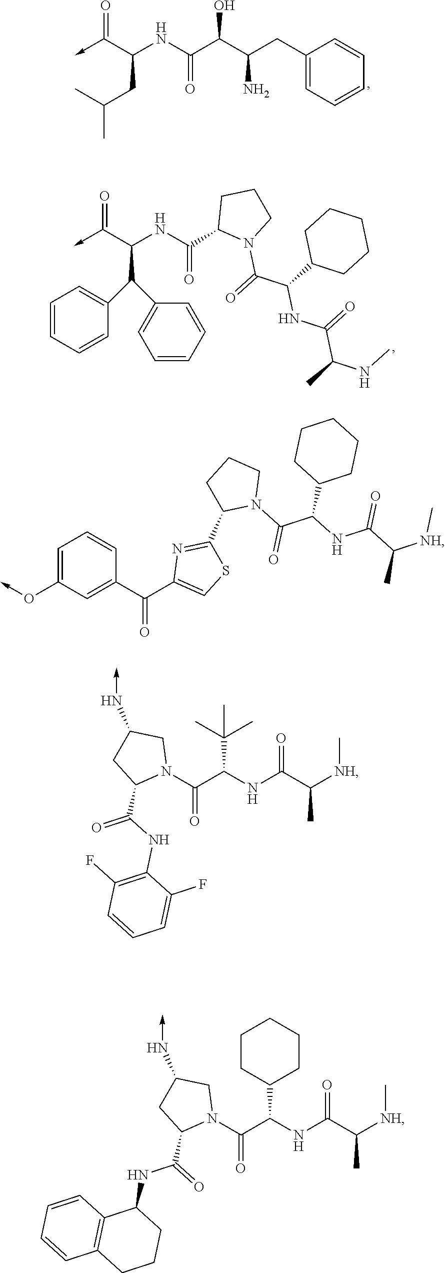

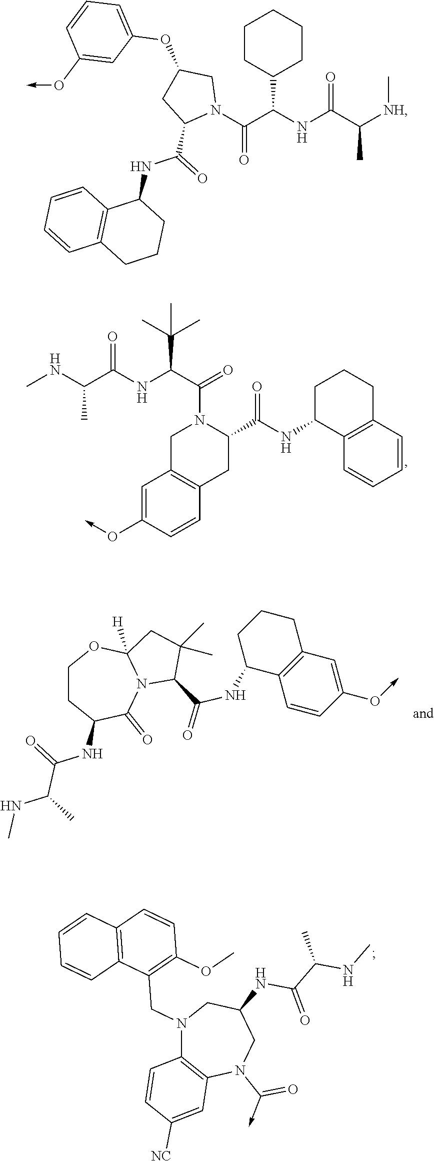

wherein the arrow denotes the covalent bond to L
or an E3 ligase binding derivative thereof.

In some embodiments, the E3 ligase binding moiety is selected from:

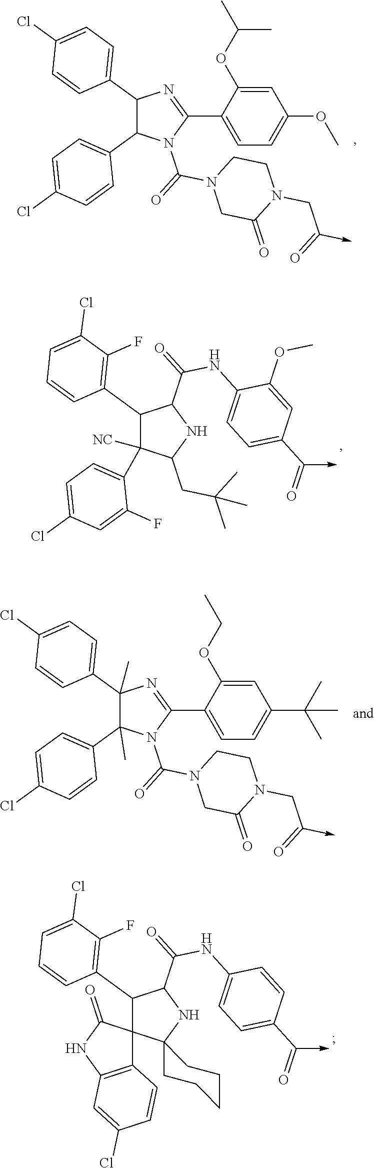

wherein the arrow denotes the covalent bond to L
or an E3 ligase binding derivative thereof.

In some embodiments, the E3 ligase binding moiety is selected from:

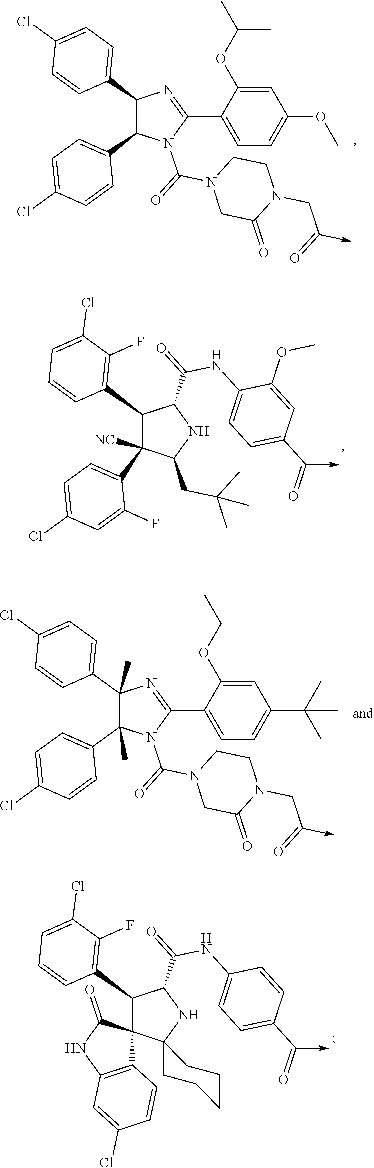

wherein the arrow denotes the covalent bond to L
or an E3 ligase binding derivative thereof.

In some embodiments, the E3 ligase binding derivative is an optionally substituted derivative of any of the E3 ligase binding moieties described herein.

### L

In the compounds of formula (X), L denotes a linker covalently linking MLKLi and E3L. Any suitable linking group may be used that it is compatible with MLKLi and E3L, does not interfere with the binding of MLKLi and E3L to their respective protein targets, and allows ubiquitin transfer from the E3 ligase to MLKL.

In some embodiments, the linker has a shortest linear chain length of 1 to 50 atoms.

As used herein “shortest linear chain length” defines the number of atoms in a chain defining the shortest path from MLKLi to E3L in a compound of the invention. For example, the shortest linear chain length in each of the following structures is 7 atoms (shortest chain length is numbered in each structure):

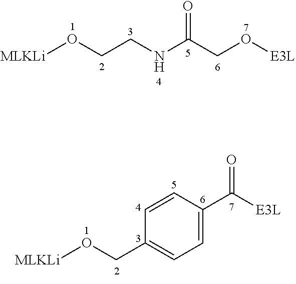

In some embodiments, the linker has a minimum shortest linear chain length of at least 1, 2, 3, 4, 5, 6, or 7 atoms. The linker may have a maximum shortest linear chain length of not more than 50, 45, 40, 35, 30, 25, 20, 15, 10, 9, 8 or 7 atoms. The linker may be characterised by a shortest linear chain length from any of these minimum lengths to any of these maximum lengths provided the minimum is less than the maximum. For example, the linker may be characterised by a shortest linear chain length of 1 to 35 atoms, 1-25 atoms, 1-20 atoms, 1-atoms, 2-10 atoms, 3-10 atoms or 5-9 atoms.

In some embodiments, the linker is a C₁₋₅₀alkyl optionally interrupted by one or more groups selected from:
a. —O—,
b. —NR^{z}—,
c. C₃₋₈cycloalkyl,
d. aryl,
e. C₁₋₄alkaryl,
f. heteroaryl,
g. (C₁₋₄alkoxy)₁₋₄aryl,
h. haloaryl,
i. 4-8-membered non-aromatic heterocyclyl,
j. —C(O)NR^{z}—,
k. alkenyl,
l. alkynyl,
wherein each R^{Z} is independently selected from H and C₁₋₄alkyl,
and wherein each of the one or more groups a-I may be further optionally substituted with a group selected from: C₃₋₆cycloalkyl, halo, —OH, —CN, —NR^{z}₂, C₁₋₄alkyl, C₁₋₄alkoxy, oxo, C₁₋₄alkylketone, —COOH, —C(O)N(R^{z})₂, and —NR^{z}C(O)R^{z}. In some embodiments, the one or more groups a-I may be optionally substituted with one or more groups selected from oxo, —C(O)N(R^{z})₂, and —NR^{z}C(O)R^{z}.

In some embodiments, each of the one or more groups a-I. may be further optionally substituted with a group selected from: halo, —OH, —CN, —NR^{z}₂, C₁₋₄alkyl, C₁₋₄alkoxy, oxo, C₁₋₄alkylketone, —COOH, —C(O)N(R^{z}), and —NR^{z}C(O)R^{z}. In some embodiments, the one or more groups a-I. may be optionally substituted with one or more groups selected from oxo, —C(O)N(R^{z})₂, and —NR^{z}C(O)R^{z}.

C₁₋₅₀alkyl may be optionally interrupted by any number of groups a-I provided the stability of the linker is sufficient to maintain the covalent connection between MLKLi and E3L under physiological conditions. Typically no more than 2 optional interrupting groups are included at consecutive positions along the C₁₋₅₀alkyl chain. In some embodiments, the C₁₋₅₀alkyl linker may be optionally interrupted by any number of groups a-I and optionally substituted.

In some embodiments, the C₁₋₅₀alkyl linker may comprise 0, 1, 2, 3, 4, 5, 6, 7, 8, 9, 10, 11, 12, 13, 14, 15, 16, 17, 18, 19 or 20 of the one or more groups a-I. In some embodiments, the C₁₋₅₀alkyl linker may comprise any number of groups a-I from any of these numbers to any other of these numbers, for example, from 1 to 20 or from 4-12 groups.

In some embodiments, the heteroaryl comprises at least one N heteroatom, such as triazolyl or pyrazolyl, preferably pyrazolyl.

The linker may comprise the moiety —(OCH₂CH₂)ᵥ—, wherein v is an integer from 1 to 15. Inclusion of the repeating ethylene oxide moiety may assist to control the hydrophilicity (and hence solubility) of the compounds of the invention. In some embodiments, the linker may comprise 1-6 ethylene glycol units, preferably 2-5 ethylene glycol units.

The linker may comprises at least one coupling moiety selected from: —C(O)O—, —C(O)NR^{z}—, —OC(O)O—, —NR^{z}C(O)NR^{z}—, —OC(O)NR^{z}—, triazolyl, aryl, α,β-unsubstituted ketone, p-hydroxy-ketone, 4-8-membered heteroaryl, unsaturated C₆-cycloalkyl and optionally substituted C₂alkenyl, wherein each R^{Z} is independently selected from H and C₁₋₄alkyl. The coupling moiety is typically the product of the reaction used to couple MLKLi with E3L. In some embodiments the coupling moiety is selected from: —C(O)O, C(O)NR^{z}—, triazolyl, aryl, 4-8 membered heteroaryl (such as pyrazolyl) and aryl.

In some embodiments, the linker is a C₁₋₅₀alkyl optionally substituted by one or more groups selected from: C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆alkoxyaryl, halo, C₁₋₆alkylhalo, C₁₋₆alkoxyhalo, carboxyl, ester, cyano, nitro, amino, substituted amino, disubstituted amino, acyl, ketone, substituted ketone, amide, aminoacyl, substituted amide, disubstituted amide, thiol, alkylthio, thioxo, sulfate, sulfonate, sulfinyl, substituted sulfinyl, sulfonyl, substituted sulfonyl, sulfonylamide, substituted sulfonamide, disubstituted sulfonamide, aryl, arC₁₋₆alkyl, heterocyclyl and heteroaryl wherein each alkyl, alkenyl, alkynyl, cycloalkyl, aryl and heterocyclyl and groups containing them may be further optionally substituted.

In some embodiments, the linker is a C₁₋₅₀alkyl optionally substituted by one or more groups selected from: C₁₋₄alkyl, C₂₋₄alkenyl, C₂₋₄alkynyl, C₃₋₄cycloalkyl, hydroxyl, oxo, C₁₋₄alkoxy, C₁₋₄alkoxyaryl, halo, C₁₋₄alkylhalo, C₁₋₄alkoxyhalo, carboxyl, ester, cyano, nitro, amino, substituted amino, disubstituted amino, acyl, ketone, substituted ketone, amide, thiol, alkylthio, thioxo, sulfate, sulfonate, sulfinyl, heterocyclyl and heteroaryl wherein each alkyl, alkenyl, alkynyl, cycloalkyl, aryl and heterocyclyl and groups containing them may be further optionally substituted.

The C₁₋₅₀alkyl may be optionally substituted by any number of groups provided the stability of the linker is sufficient to maintain the covalent connection between MLKLi and E3L under physiological conditions. Typically no more than 2 optional substituting groups are included at consecutive positions along the C₁₋₅₀alkyl chain.

Typically, the compounds of the invention may be prepared by techniques known in the art.

In another aspect, there is also provided a process for preparing a compound of formula (I) or a salt, solvate, tautomer, N-oxide, stereoisomer and/or prodrug thereof. In some embodiments, the process comprises any of the following 4 steps:
reacting a compound of formula (V) with a compound of formula (VI)

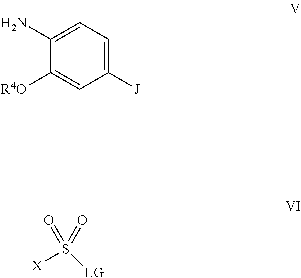

wherein X, J and R⁴ are as defined for formula (I) and
LG is a leaving group, such as halo. The leaving group may be any that is capable of activating the sulphonyl moiety of the compound of formula (VI) as an electrophile capable of reacting under appropriate conditions with the free aniline nitrogen of the compound of formula (V).
reacting a compound of formula (VII) with a compound of formula (VIII)

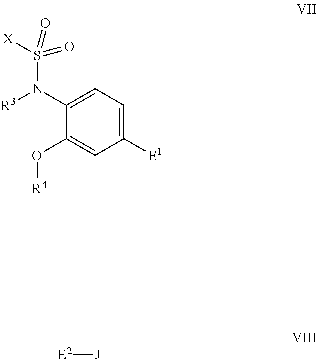

wherein X, R³, R⁴ and J are as defined for formula (I) or any embodiment thereof; E¹ and E² are an electrophilic pair suitable to direct a palladium catalysed cross-coupling reaction. In some embodiments, E¹ and E² are selected from halo, a boronic acid, a boronic ester and triflate, or a group capable of conversion to any of these moieties, such as hydroxyl, a mixed anhydride, tetrafluorophenyl ether, etc. In some embodiments, E¹ is halo, and E² is selected from a boronic acid, a boronic ester and triflate. In some embodiments, E² is halo, and E¹ is selected from a boronic acid, a boronic ester and triflate;
reacting a compound of formula (IX) with a compound of formula (X)

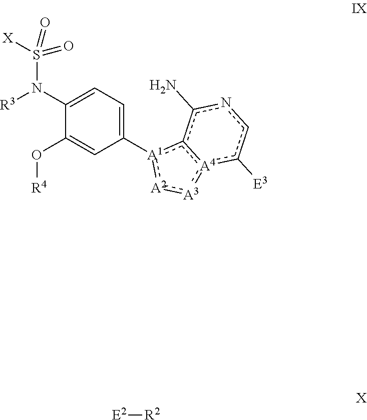

wherein X, R², R³, R⁴, A¹, A², A³ and A⁴ are as defined for formula (I) or any embodiment thereof; E³ and E⁴ are an electrophilic pair suitable to direct a palladium catalysed cross-coupling reaction. In some embodiments, E³ and E⁴ are selected from halo, a boronic acid, a boronic ester and triflate, or a group capable of conversion to any of these moieties, such as hydroxyl, a mixed anhydride, tetrafluorophenyl ether, etc. In some embodiments, E³ is halo, and E⁴ is selected from a boronic acid, a boronic ester and triflate. In some embodiments, E⁴ is halo, and E³ is selected from a boronic acid, a boronic ester and triflate;
converting a compound of formula (I) into one of its salts.

In some embodiments of the above process, reactive moieties in the compounds of formulas (V)—(X) are functionalised with an appropriate protecting group.

Embodiments of these steps are shown in the General Schemes below.

In some embodiments, the process comprises a reaction with one or more of formula (XIII), formula (XIV) and formula (XV)

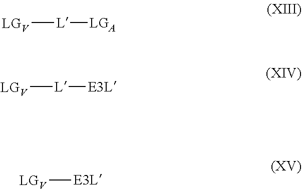

wherein
L′ is a linker moiety or a protected form of a linker moiety;
E3L′ is an E3 ligase binding moiety or a protected form of an E3 ligase binding moiety;
LGᵥ is a group cleavable in the reaction with its coupling partner, wherein the coupling partner is selected from a compound of formula (I), formula (IA), formula (IB), formula (IA′), formula (IB′), formula (II), formula (III), formula (IV), formula (V), formula (VI), formula (VII) and formula (VIII); and
LG_{A} is H or a group cleavable in a subsequent step reacting moiety L′ with E3L′.

In some embodiments, the process comprises reacting a compound of formula (I) with a compound selected from the group consisting of formula (XIII), formula (XIV) and formula (XV).

In some embodiments, the process comprises reacting a compound of formula (IA) with a compound selected from the group consisting of formula (XIII), formula (XIV) and formula (XV).

In some embodiments, the process comprises reacting a compound of formula (IB) with a compound selected from the group consisting of formula (XIII), formula (XIV) and formula (XV).

In some embodiments, the process comprises reacting a compound of formula (IA′) with a compound selected from the group consisting of formula (XIII), formula (XIV) and formula (XV).

In some embodiments, the process comprises reacting a compound of formula (I B′) with a compound selected from the group consisting of formula (XIII), formula (XIV) and formula (XV).

In some embodiments, the process comprises reacting a compound of formula (II) with a compound selected from the group consisting of formula (XIII), formula (XIV) and formula (XV).

In some embodiments, the process comprises reacting a compound of formula (III) with a compound selected from the group consisting of formula (XIII), formula (XIV) and formula (XV).

In some embodiments, the process comprises reacting a compound of formula (IV) with a compound selected from the group consisting of formula (XIII), formula (XIV) and formula (XV).

In some embodiments, the process comprises reacting a compound of formula (V) with a compound selected from the group consisting of formula (XIII), formula (XIV) and formula (XV).

In some embodiments, the process comprises reacting a compound of formula (VI) with a compound selected from the group consisting of formula (XIII), formula (XIV) and formula (XV).

In some embodiments, the process comprises reacting a compound of formula (VII) with a compound selected from the group consisting of formula (XIII), formula (XIV) and formula (XV).

In some embodiments, the process comprises reacting a compound of formula (VIII) with a compound selected from the group consisting of formula (XIII), formula (XIV) and formula (XV).

In some embodiments, L′ may be deprotected before deprotection of E3L′. In some embodiments, L′ may be deprotected subsequent to deprotection of E3L′.

In some embodiments, L′ may be deprotected before coupling with the coupling partner. In some embodiments, L′ may be deprotected subsequent to coupling with the coupling partner.

In some embodiments, L′ may be deprotected before cleavage of LG_{A}. In some embodiments, L′ may be deprotected subsequent to cleavage of LG_{A}.

In some embodiments, E3L′ may be deprotected before coupling with the coupling partner.

In some embodiments, E3L′ may be deprotected subsequent to coupling with the coupling partner.

In some embodiments, E3L′ may be deprotected before cleavage of LG_{A}. In some embodiments, E3L′ may be deprotected subsequent to cleavage of LG_{A}.

In some embodiments, LG_{A} is cleaved prior to coupling with the coupling partner. In preferred embodiments where LG_{A} is cleaved prior to coupling with the coupling partner, the process comprises reacting a compound of formula (XIII) with a compound of formula (XIV), thereby forming a compound of formula (XV). In some embodiments, LG_{A} is cleaved subsequent to coupling with the coupling partner.

In some embodiments, the process involving a reaction with one or more of formula (XIII), formula (XIV) and formula (XV) comprises a palladium mediated cross-coupling reaction.

In some embodiments, the process involving a reaction with one or more of formula (XIII), formula (XIV) and formula (XV) comprises deprotection of an amino protein group.

In some embodiments of the above process, wherein PG¹ is an amino protecting group, the process further comprises a deprotection step.

Embodiments of these steps are shown in the syntheses below.

The specific reagents and conditions for effecting each of these steps will depend on the specific substituents selected for each reaction partner. The skilled person would readily appreciate how to determine and/or optimise these reagents and conditions. Similarly, where a starting material is not commercially available, the skilled person would be able to design and implement its preparation based on techniques and reactions previously described. Embodiments of these steps are provided in the Examples with reference to specific compounds described herein.

### Methods

In another aspect, there is provided a method for inhibiting necroptosis in a subject in need thereof, the method comprising administering a therapeutically effective amount of a compound according to Formula (I) or a pharmaceutically acceptable salt, solvate, tautomer, N-oxide, stereoisomer and/or prodrug thereof.

Without wishing to be bound by theory, it is believed that the compounds of the invention treat necroptosis by binding to the ATP-binding site of the pseudokinase domain of Mixed Lineage Kinase Domain-like (MLKL) protein and triggering its ubiquitination and protein degradation via the ubiquitin-proteasome pathway.

As such, in another aspect, there is a method of degrading mixed lineage kinase domain-like protein (MLKL). As used herein in the context of this invention, the terms “degrading” and “degradation” would be understood by the person skilled in the art to mean partial or complete proteolysis of the protein via the ubiquitin-proteasome pathway. The E³ ubiquitin ligase, prompts transfer of ubiquitin from an E² ubiquitin conjugating enzyme, leading to ubiquitination of the target protein and degradation by the proteasomes.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

In one embodiment of the present disclosure, administration of a compound according to Formula (I) inhibits a conformational change of MLKL. In another embodiment, the conformational change of MLKL involves release of the four-helix bundle (4HB) domain of MLKL. In another embodiment, administration of the compound inhibits oligomerisation of MLKL. In yet another embodiment, administration of the compound inhibits translocation of MLKL to the cell membrane. In a further embodiment, administration of the compound inhibits a conformational change of MLKL, inhibits oligomerisation of MLKL and inhibits translocation of MLKL to the cell membrane.

It is envisaged that some compounds of the present disclosure can bind to MLKL in various species and inhibit necroptosis.

As used herein, the term “pseudokinase domain” as understood by a person skilled in the art, means a protein containing a catalytically-inactive or catalytically-defective kinase domain. “Pseudokinase domains” are often referred to as “protein kinase-like domains” as these domains lack conserved residues known to catalyse phosphoryl transfer. It would be understood by a person skilled in the art that although pseudokinase domains are predicted to function principally as catalysis independent protein-interaction modules, several pseudokinase domains have been attributed unexpected catalytic functions. Accordingly, in the present disclosure the term “pseudokinase domain” includes “pseudokinase domains” which lack kinase activity and “pseudokinase domains” which possess weak kinase activity.

As used herein, the term “ATP-binding site” as understood by a person skilled in the art, means a specific sequence of protein subunits that promotes the attachment of ATP to a target protein. An ATP binding site is a protein micro-environment where ATP is captured and hydrolyzed to ADP, thereby releasing energy that is utilized by the protein to work by changing the protein shape and/or making the enzyme catalytically active. In pseudokinase domains, the “ATP-binding site” is often referred to as the “pseudoactive site”. The term “ATP-binding site” may also be referred to as a “nucleotide-binding site” as binding at this site includes the binding of nucleotides other than ATP. It would be understood by a person skilled in the art that the term “nucleotide” includes any nucleotide. Exemplary nucleotides include, but are not limited to, AMP, ADP, ATP, AMPPNP, GTP, CTP and UTP.

As described herein, inhibition of necroptosis includes both complete and partial inhibition of necroptosis. In one embodiment, inhibition of necroptosis is complete inhibition. In another embodiment, inhibition of necroptosis is partial inhibition.

Binding of the compound to the ATP-binding site of the pseudokinase domain of MLKL may inhibit phosphorylation of MLKL by an effector kinase or binding of the compound to the ATP-binding site of the pseudokinase domain of MLKL may not inhibit phosphorylation of MLKL by an effector kinase. The present disclosure demonstrates that compounds that bind to the ATP-binding site of the pseudokinase domain of the MLKL protein, as described herein, can inhibit necroptosis without inhibiting phosphorylation of MLKL by an effector kinase. In one embodiment, binding of the compound to the ATP-binding site of the pseudokinase domain of MLKL does not inhibit phosphorylation of MLKL by an effector kinase. In another embodiment, binding of the compound to the ATP-binding site of the pseudokinase domain of MLKL inhibits phosphorylation of MLKL by an effector kinase. RIP1, RIP3 and MLKL are three proteins implicated in the necroptotic pathway. Upon necroptotic stimulus (e.g. using the combination of TNF, SMAC mimetic and QVD-OPh on suitable cell lines), RIP1 is auto-phosphorylated leading to association with RIP3, which in turn auto-phosphorylates itself. Activated RIP3 phosphorylates MLKL leading to a putative conformational change that triggers its necroptotic activity (Murphy, Immunity, 39, pp 443-453, 2013). MLKL acts downstream of RIP1 and RIP3, and is therefore understood to be a key effector of necroptosis. Compounds of this invention may bind to MLKL and block this conformational change or any other key event in its activation.

The compounds of the invention may be selective for MLKL. In some embodiments, the compounds of the invention are selective for MLKL over RIP1. In some embodiments, the compounds of the invention are selective for MLKL over RIP3. In some embodiments, the compounds of the invention are selective for MLKL over RIP1 and RIP3. A selective compound may have 5-fold, 10-fold, 50-fold, 100-fold, 500-fold, 1000-fold or greater selectivity for MLKL compared to RIP1 and/or RIP3. Typically, the relative selectivity may be assessed by comparing K_{D} values for each respective compound binding to the relevant protein (ie MLKL and either or both of RIP1 and RIP3). Suitable assay conditions are described in the Examples below. Compounds selective for MLKL may avoid undesired side-effects associated with RIP1 and/or RIP3 loss of function.

In another aspect, there is provided use of a compound of Formula (X) a pharmaceutically acceptable salt, solvate, tautomer, N-oxide, stereoisomer and/or prodrug thereof in the preparation of a medicament for the inhibition of necroptosis in a subject.

In another aspect, there is provided use of a composition comprising a compound of Formula (X) or a pharmaceutically acceptable salt, solvate, tautomer, N-oxide, stereoisomer and/or prodrug thereof for the inhibition of necroptosis in a subject.

In another aspect, there is provided use of a compound of Formula (X) or a pharmaceutically acceptable salt, solvate, tautomer, N-oxide, stereoisomer and/or prodrug thereof for inhibiting necroptosis.

In another aspect, there is provided use of a composition comprising a compound of Formula (X) or a salt, solvate, tautomer, N-oxide, stereoisomer and/or prodrug thereof for inhibiting necroptosis.

In yet another aspect, there is provided a compound according to Formula (X) or a salt, solvate, tautomer, N-oxide, stereoisomer and/or prodrug thereof for use in inhibiting necroptosis.

In yet another aspect, there is provided a composition comprising a compound according to Formula (X) or a pharmaceutically acceptable salt, solvate, tautomer, N-oxide, stereoisomer and/or prodrug thereof for use in inhibiting necroptosis. In some embodiments, the composition is a pharmaceutical composition.

In yet another aspect, there is provided a compound according to Formula (X) or a pharmaceutically acceptable salt, solvate, tautomer, N-oxide, stereoisomer and/or prodrug thereof when used for inhibiting necroptosis.

In yet another aspect, there is provided a composition comprising a compound according to Formula (X) or a pharmaceutically acceptable salt, solvate, tautomer, N-oxide, stereoisomer and/or prodrug thereof when used for inhibiting necroptosis.

In another aspect, there is provided a method of inhibiting MLKL, comprising contacting a cell with an effective amount of a compound of formula (X) or a salt, solvate, tautomer, N-oxide, stereoisomer and/or prodrug thereof.

The salts of the compounds of Formula (X) are preferably pharmaceutically acceptable, but it will be appreciated that non-pharmaceutically acceptable salts also fall within the scope of the present disclosure, for example, as these may be useful as intermediates in the preparation of pharmaceutically acceptable salts or in methods not requiring administration to a subject.

The term “pharmaceutically acceptable” may be used to describe any salt, solvate, tautomer, N-oxide, stereoisomer and/or prodrug thereof, or any other compound which upon administration to a subject, is capable of providing (directly or indirectly) a compound of Formula (X) or an active metabolite or residue thereof and typically that is not deleterious to the subject.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae.

The invention includes all crystalline forms of a compound of Formula (X) including anhydrous crystalline forms, hydrates, solvates and mixed solvates. If any of these crystalline forms demonstrates polymorphism, all polymorphs are within the scope of this invention.

Formula (X) is intended to cover, where applicable, solvated as well as unsolvated forms of the compounds. Thus, Formula (X) includes compounds having the indicated structures, including the hydrated or solvated forms, as well as the non-hydrated and non-solvated forms.

The compounds of Formula (X) or salts, tautomers, N-oxides, polymorphs or prodrugs thereof may be provided in the form of solvates. Solvates contain either stoichiometric or non-stoichiometric amounts of a solvent, and may be formed during the process of crystallization with pharmaceutically acceptable solvents such as water, alcohols such as methanol, ethanol or isopropyl alcohol, DMSO, acetonitrile, dimethyl formamide (DMF), acetic acid, and the like with the solvate forming part of the crystal lattice by either non-covalent binding or by occupying a hole in the crystal lattice. Hydrates are formed when the solvent is water, alcoholates are formed when the solvent is alcohol. Solvates of the compounds of the present invention can be conveniently prepared or formed during the processes described herein. In general, the solvated forms are considered equivalent to the unsolvated forms for the purposes of the invention.

Basic nitrogen-containing groups may be quarternised with such agents as C₁₋₆alkyl halide, such as methyl, ethyl, propyl, and butyl chlorides, bromides and iodides; dialkyl sulfates like dimethyl and diethyl sulfate; and others.

Nitrogen containing groups may also be oxidised to form an N-oxide.

The compound of Formula (X) or salts, tautomers, N-oxides, solvates and/or prodrugs thereof that form crystalline solids may demonstrate polymorphism. All polymorphic forms of the compounds, salts, tautomers, N-oxides, solvates and/or prodrugs are within the scope of the invention.

The compound of Formula (I) (and therefore also the compound of formula (X)) may demonstrate tautomerism. Tautomers are two interchangeable forms of a molecule that typically exist within an equilibrium. Any tautomers of the compounds of Formula (I) are to be understood as being within the scope of the invention when included in a compound of the invention as moiety MLKLi. For example, when R¹ is H the compounds of formula (IA) and (1B) may exist as tautomers, eg in equilibrium with each other. The compounds of formula (IA) and (1B) wherein R¹ is H are depicted below as compounds of formulas (IA′) and (1B′). The proportion of compounds of formula (IA′) to (1B′) in equilibrium may depend on the specific compound and conditions, such as solvent, temperature, concentration, etc. This equilibrium may be described as follows:

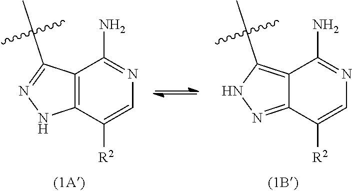

Similar tautomerism may occur for any pyrazole-containing compound described herein, including compounds of formula (II), (Ill), (IV), (VIII), (IX), (XX), (XXI), (XXII), (XXIII) and (XXIV), compounds 1-173 and compounds 1001-1145. All tautomers of these compounds are contemplated and considered within the scope of the present invention. In addition, further tautomeric forms may exist for the compounds described herein for example depending on various substituents selected.

The compound of Formula (X) may contain one or more stereocentres. All stereoisomers of the compounds of formula (X) are within the scope of the invention. Stereoisomers include enantiomers, diastereomers, geometric isomers (E and Z olephinic forms and cis and trans substitution patterns) and atropisomers. In some embodiments, the compound is a stereoisomerically enriched form of the compound of formula (X) at any stereocentre. The compound may be enriched in one stereoisomer over another by at least about 60, 70, 80, 90, 95, 98 or 99%.

The compound of Formula (X) or its salts, tautomers, solvates, N-oxides, and/or stereoisomers, may be isotopically enriched with one or more of the isotopes of the atoms present in the compound. For example, the compound may be enriched with one or more of the following minor isotopes: ²H, ³H, ¹³C, ¹⁴C, ¹⁵N and/or¹⁷0, preferably ²H. An isotope may be considered enriched when its abundance is greater than its natural abundance.

A “prodrug” is a compound that may not fully satisfy the structural requirements of the compounds provided herein, but is modified in vivo, following administration to a subject or patient, to produce a compound of formula (X) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of compounds of Formula (X). The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of Formula (X) through the carbonyl carbon prodrug sidechain.

Pharmaceutical compositions may be formulated from compounds according to Formula (X) for any appropriate route of administration including, for example, oral, rectal, nasal, vaginal, topical (including transdermal, buccal, ocular and sublingual), parenteral (including subcutaneous, intraperitoneal, intradermal, intravascular (for example, intravenous), intramuscular, spinal, intracranial, intrathecal, intraocular, periocular, intraorbital, intrasynovial and intraperitoneal injection, intracisternal injection as well as any other similar injection or infusion techniques), inhalation, insufflation, infusion or implantation techniques (e.g., as sterile injectable aqueous or non-aqueous solutions or suspensions).

In certain embodiments, compositions in a form suitable for oral use or parenteral use are preferred. Suitable oral forms include, for example, tablets, troches, lozenges, aqueous or oily suspensions, dispersible powders or granules, emulsions, hard or soft capsules, or syrups or elixirs. For intravenous, intramuscular, subcutaneous, or intraperitoneal administration, one or more compounds may be combined with a sterile aqueous solution which is preferably isotonic with the blood of the recipient. Such formulations may be prepared by dissolving solid active ingredient in water containing physiologically compatible substances such as sodium chloride or glycine, and having a buffered pH compatible with physiological conditions to produce an aqueous solution, and rendering said solution sterile. The formulations may be present in unit or multi-dose containers such as sealed ampoules or vials. Examples of components are described in Martindale—The Extra Pharmacopoeia (Pharmaceutical Press, London 1993), and Remington: The Science and Practice of Pharmacy, 21st Ed., 2005, Lippincott Williams & Wilkins. All methods include the step of bringing the active ingredient, for example a compound defined by Formula (X), or a pharmaceutically acceptable salt or prodrug thereof, into association with the carrier which constitutes one or more accessory ingredients. In general, the pharmaceutical compositions are prepared by uniformly and intimately bringing the active ingredient, for example a compound defined by Formula (X), or a pharmaceutically acceptable salt or prodrug thereof, into association with a liquid carrier or a finely divided solid carrier or both, and then, if necessary, shaping the product into the desired formulation. In the pharmaceutical composition the active object compound is included in an amount sufficient to produce the desired effect. In some embodiments, the method of the invention comprises administering a pharmaceutical comprising a compound of Formula (X) or a pharmaceutically acceptable salt or solvate thereof and a pharmaceutically acceptable carrier, diluent and/or excipient.

In the context of this specification the term “administering” and variations of that term including “administer” and “administration”, includes contacting, applying, delivering or providing a compound or composition of the invention to an organism, or a surface by any appropriate means.

For the inhibition of necroptosis, the dose of the biologically active compound according to the invention may vary within wide limits and may be adjusted to individual requirements. Active compounds according to the present invention are generally administered in a therapeutically effective amount. The daily dose may be administered as a single dose or in a plurality of doses. The amount of active ingredient that may be combined with the carrier materials to produce a single dosage form will vary depending upon the subject treated and the particular mode of administration.

It will be understood, however, that the specific dose level for any particular subject will depend upon a variety of factors including the activity of the specific compound employed, the age, body weight, general health, sex and diet of the subject, time of administration, route of administration, and rate of excretion, drug combination (i.e. other drugs being used to treat the subject), and the severity of the particular disorder undergoing therapy. Such treatments may be administered as often as necessary and for the period of time judged necessary by the treating physician. A person skilled in the art will appreciate that the dosage regime or therapeutically effective amount of the compound of formula (X) to be administered may need to be optimized for each individual.

It will also be appreciated that different dosages may be required for treating different disorders. An effective amount of an agent is that amount which causes a statistically significant decrease in necroptosis.

For in vitro analysis, the necroptosis inhibition may be determined by assays used to measure TSQ-induced necroptosis, as described in the biological tests defined herein.

The terms “treating”, “treatment” and “therapy” are used herein to refer to curative therapy, prophylactic therapy and preventative therapy. Thus, in the context of the present disclosure the term “treating” encompasses curing, ameliorating or tempering the severity of necroptosis and/or associated diseases or their symptoms.

“Preventing” or “prevention” means preventing the occurrence of the necroptosis or tempering the severity of the necroptosis if it develops subsequent to the administration of the compounds or pharmaceutical compositions of the present invention.

“Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

The term “inhibit” is used to describe any form of inhibition that results in prevention, reduction or otherwise amelioration of necroptosis and/or MLKL function, including complete and partial inhibition.

The term “degrade” is used to describe any degree of degradation of the target protein that results in diminished function of MLKL and otherwise amelioration of necroptosis. In some embodiments, a compound of the invention trigger substantially complete degradation of the target MLKL protein to which it binds. Accordingly, also described herein are methods of degrading MLKL in a subject, comprising administering to the subject a compound of the invention.

As the compounds of the invention include both an MLKL binding moiety—MLKLi—that is based on a series of MLKL inhibitors described in AU 2021904206 (entirely incorporated herein by reference), compounds of the invention may both inhibit and degrade MLKL, which may enhance the amelioration of necroptosis in a subject.

The compounds of the present invention may be administered along with a pharmaceutical carrier, diluent and/or excipient as described above.

The methods of the present disclosure can be used to prevent or treat the following disease(s), condition(s) and/or disorder(s) in a subject:
diseases of the bones, joints, connective tissue and of cartilage, such as osteoporosis, osteomyelitis including chronic recurrent multifocal osteomyelitis, arthritises including for example osteoarthritis, rheumatoid arthritis and psoriatic arthritis, avascular necrosis, progressive fibrodysplasia ossificans, rickets, Cushing's syndrome;
muscular diseases such as muscular dystrophy, such as for example Duchenne's muscular dystrophy, myotonic dystrophies, myopathies and myasthenias;
diseases of the skin, such as dermatitis, eczema, psoriasis, aging or even alterations of scarring;
cardiovascular diseases such as cardiac and/or vascular ischemia, myocardial infarction, ischemic cardiopathy, chronic or acute congestive heart failure, cardiac dysrythmia, atrial fibrillation, ventricular fibrillation, paroxystic tachycardia, congestive heart failure, hypertrophic cardiopathy, anoxia, hypoxia, secondary effects due to therapies with anti-cancer agents;
circulatory diseases such as atherosclerosis, arterial scleroses and peripheral vascular diseases, strokes including cerebrovascular strokes, aneurisms;
haematological and vascular diseases such as: anemia, aplastic anemia, vascular amyloidosis, haemorrhages, drepanocytosis, red cell fragmentation syndrome, neutropenia, leukopenia, medullar aplasia, pantocytopenia, thrombocytopenia, haemophilia;
lung diseases including pneumonia, asthma; obstructive chronic diseases of the lungs such as for example chronic obstructive pulmonary disease (COPD), chronic bronchitis and emphysema;
diseases of the gastro-intestinal tract, such as ulcers; inflammatory bowel diseases (IBD), including Crohn's disease, ulcerative colitis;
diseases of the liver such as for example hepatitis particularly hepatitis of viral origin or having as causative agent other infectious agents, auto-immune hepatitis, fulminating hepatitis, inflammatory hepatitis, certain hereditary metabolic disorders, Wilson's disease, cirrhoses, non-alcoholic fatty liver disease (NAFLD) including non-alcoholic hepatic steatosis and/or non-alcoholic steatohepatitis (NASH), diseases of the liver due to toxins and to drugs such as drug-induced liver injury, ethanol (or alcohol)-induced liver disease;
diseases of the pancreas such as for example acute or chronic pancreatitis;
metabolic diseases such as diabetes, including diabetes mellitus, pre-diabetes and insipid diabetes; thyroiditis;
diseases of the kidneys such as acute renal disorders (such as acute kidney injury (AKI), including ischaemic reperfusion injury (IRI)) or glomerulonephritis;
viral and bacterial infections such as septicemia;
severe intoxications by chemicals, toxins or drugs;
degenerative diseases associated with the Acquired Immune Deficiency Syndrome (AIDS);
disorders associated with aging such as the syndrome of accelerated aging;
inflammatory diseases such as Terminal ileitis including Crohn's disease, rheumatoid polyarthritis, TNF-induced systemic inflammatory syndrome;
auto-immune diseases such as erythematous lupus (including systemic lupus erythematosus), cleavage-resistant RIPK1-induced autoinflammatory (CRIA) syndrome;
dental disorders such as those resulting in degradation of tissues such as for example periodontitis;
ophthalmic diseases or disorders including diabetic retinopathies, glaucoma, macular degenerations, retinal degeneration, retinitis pigmentosa, retinal holes or tears, retinal detachment, retinal ischemia, acute retinopathies associated with trauma, inflammatory degenerations, post-surgical complications, medicinal retinopathies, cataract, cone cell degeneration;
disorders of the audition tracts, such as otosclerosis and deafness induced by antibiotics;
Ischemic reperfusion injury, including retinal ischaemic reperfusion injury;
Neuronal loss, including Alzheimer's disease and neurodegenerative diseases such as amyotrophic lateral sclerosis (ALS; also referred to as motor neuron disease (MND) and Charcot disease);
diseases associated with mitochondria (mitochondrial pathologies), such as Friedrich's ataxia, congenital muscular dystrophy with structural mitochondrial abnormality, certain myopathies (MELAS syndrome, MERFF syndrome, Pearson's syndrome), MIDD (mitochondrial diabetes and deafness) syndrome, Wolfram's syndrome, dystonia;
cancer and metastasis including but not limited to cancers of the lung and bronchus, including non-small cell lung cancer (NSCLC), squamous lung cancer, brochioloalveolar carcinoma (BAC), adenocarcinoma of the lung, and small cell lung cancer (SCLC); prostate cancer, including androgen-dependent and androgen-independent prostate cancer; breast cancer, including metastatic breast cancer; pancreatic cancer; cancers of the colon and rectum; thyroid cancer; cancers of the liver and intrahepatic bile duct; hepatocellular cancer; gastric cancer; endometrial cancer; melanoma; cancers of the kidney, renal pelvis, urinary bladder, uterine corpus and uterine cervix; ovarian cancer, including progressive epithelial or primary peritoneal cancer; multiple myeloma; oesophageal cancer, including squamous cell carcinoma and adenocarcinoma of the oesophagus; acute myelogenous leukemia (AML); chronic myelogenous leukemia (CML), including accelerated CML and CML blast phase (CML-BP); lymphocytic leukemia; myeloid leukemia; acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma, including diffuse large B-cell lymphoma (DLBCL); T-cell lymphoma; multiple myeloma (MM); amyloidosis; Waldenstrom's macroglobulinemia;
myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes; cancers of the brain, including glioma/glioblastoma, anaplastic oligodendroglioma, and adult anaplastic astrocytoma; neuroendocrine cancers, including metastatic neuroendocrine tumors; cancers of the head and neck, including, e.g., squamous cell carcinoma of the head and neck, and nasopharyngeal cancer; cancers of the oral cavity, pharynx and small intestine; bone cancer; soft tissue sarcoma; and villous colon adenoma; and
diseases of the central nervous system (CNS), such as multiple sclerosis (MS).

In some embodiments, the methods of the present disclosure may be for treating and/or preventing any one or more of the diseases, conditions and/or disorders disclosed herein. For example, in some embodiments, there is provided a method for treating and/or preventing any one or more of: retinal ischaemic reperfusion injury, chronic recurrent multifocal osteomyelitis, aplastic anaemia, CRIA, ethanol-induced liver disease, NASH, inflammatory hepatitis, acute kidney injury, IRI, multiple sclerosis, amyotrophic lateral sclerosis, Alzheimer's disease, stroke, systemic lupus erythematosus, myocardial infarction, diabetes, Crohn's disease, inflammatory bowel disease and COPD, comprising administering to a subject in need thereof an effective amount of a compound of the invention or a pharmaceutically acceptable salt, solvate, tautomer, N-oxide, stereoisomer and/or prodrug thereof.

The methods can also be used for protecting cells, tissues and/or transplanted organs, whether before, during (removal, transport and/or re-implantation) or after transplantation.

In some embodiments, the compound of the invention may be administered in combination with a further active pharmaceutical ingredient (API). The API may be any that is suitable for treating any of the diseases, conditions and/or disorders associated with necroptosis, such as those described herein. The compound of the invention may be co-formulated with the further API in any of the pharmaceutical compositions described herein, or the compound of the invention may be administered in a concurrent, sequential or separate manner. Concurrent administration includes administering the compound of the invention at the same time as the other API, whether coformulated or in separate dosage forms administered through the same or different route. Sequential administration includes administering, by the same or different route, the compound of the invention and the other API according to a resolved dosage regimen, such as within about 0.5, 1, 2, 3, 4, 5, or 6 hours of the other. When sequentially administered, the compound of the invention may be administered before or after administration of the other API. Separate administration includes administering the compound of the invention and the other API according to regimens that are independent of each other and by any route suitable for either active, which may be the same or different.

The methods may comprise administering the compound of Formula (X) in any pharmaceutically acceptable form. In some embodiments, the compound of Formula (X) is provided in the form of a pharmaceutically acceptable salt, solvate, N-oxide, polymorph, tautomer or prodrug thereof, or a combination of these forms in any ratio.

The methods may also comprise administering a pharmaceutical composition comprising the compound of formula (X) or a pharmaceutically acceptable salt, solvate, N-oxide, polymorph, tautomer or prodrug thereof to the subject in need thereof. The pharmaceutical composition may comprise any pharmaceutically acceptable carrier, diluent and/or excipient described herein.

The compounds of Formula (X), or a pharmaceutically acceptable salt or prodrug thereof, as defined herein, may be administered by any suitable means, for example, orally, rectally, nasally, vaginally, topically (including buccal and sub-lingual), parenterally, such as by subcutaneous, intraperitoneal, intravenous, intramuscular, or intracisternal injection, inhalation, insufflation, infusion or implantation techniques (e.g., as sterile injectable aqueous or non-aqueous solutions or suspensions).

The compounds of the invention may be provided as pharmaceutical compositions including those for oral, rectal, nasal, topical (including buccal and sub-lingual), parenteral administration (including intramuscular, intraperitoneal, sub-cutaneous and intravenous), or in a form suitable for administration by inhalation or insufflation. The compounds of Formula (X), or a pharmaceutically acceptable salt or prodrug thereof, together with a conventional adjuvant, carrier or diluent, may thus be placed into the form of pharmaceutical compositions and unit dosages thereof, and in such form may be employed as solids, such as tablets or filled capsules, or liquids as solutions, suspensions, emulsions, elixirs or capsules filled with the same, all for oral use, or in the form of sterile injectable solutions for parenteral (including subcutaneous) use.

Also provided is a kit of parts, comprising in separate parts:
a compound of Formula (X) or a pharmaceutically acceptable salt, solvate, N-oxide, polymorph, tautomer or prodrug thereof; and
instructions for its use in any of the methods of the invention.

### EXAMPLES

The compounds, compositions, kits and methods described herein are described by the following illustrative and non-limiting examples.

### Chemistry

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents, which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., 1H or 13C), infrared spectroscopy, spectrophotometry (e.g., UV-visible), or mass spectrometry, or by chromatography such as high-performance liquid chromatography (HPLC) or thin layer chromatography.

The expressions, “ambient temperature,” “room temperature,” and “RT”, as used herein, are understood in the art, and refer generally to a temperature, e.g. a reaction temperature, that is about the temperature of the room in which the reaction is carried out, for example, a temperature from about 20° C. to about 30° C.

Compounds of the invention can be prepared according to numerous preparatory routes known in the literature. Example synthetic methods for preparing compounds of the invention are provided in the Schemes below.

### General Chemistry Methods

### Definitions

AcCl (acetyl chloride);
Ac₂O (acetic anhydride);
AcOH (acetic acid);
atm (atmosphere);
B₂pin₂ (bis(pinacolato)diboron);
BINAP (2,2′-bis(diphenylphosphino)-1,1′-binaphthyl);
BnBr (benzyl bromide);
Boc₂O (di-tert-butyl dicarbonate);
c-Hex (cyclohexane);
CDCl₃ (deuterated chloroform);
CD₃OD (deuterated methanol);
CHCl₃ (chloroform);
Cs₂CO₃ (cesium carbonate)
CsF (cesium fluoride);
conc. (concentrated);
d (day);
DAST (diethylaminosulphur trifluoride);
dba (dibenzylideneacetone);
DCM (dichloromethane);
DEAD (diethyl azodicarboxylate);
DIAD (diisopropyl azodicarboxylate);
DIPEA (N,N-diisopropylethylamine);
DMAP (4-dimethylaminopyridine);
DME (1,2-dimethoxyethane);
DMF (N,N-dimethylformamide);
DMSO (dimethyl sulfoxide);
DMSO-d₆ (deuterated dimethyl sulfoxide);
EDCI (1-ethyl-3-(3-dimethylaminopropyl)carbodiimide);
eq (equivalent);
Et₃N (triethylamine);
Et₂O (diethyl ether);
EtOAc (ethyl acetate);
EtOH (ethanol);
EtSO₂Cl (ethanesulfonyl chloride);
g (gram);
h (hour);
H₂ (hydrogen);
HATU (1-[bis(dimethylamino)methylene]-1H-1,2,3-triazolo[4,5-b]pyridinium 3-oxide hexafluorophosphate);
HCl (hydrochloric acid/hydrogen chloride);
HOBt (hydroxybenzotriazole);
¹H NMR (proton nuclear magnetic resonance);
Hz (hertz);
i-PrOH (iso-propanol);
KF (potassium fluoride);
L (litre);
LCMS (liquid chromatography-mass spectrometry);
LiHMDS (lithium bis(trimethylsilyl)amide);
M (molar);
MeCN (acetonitrile);
Me₂NH (dimethylamine);
MeOH (methanol);
MeOD-d₄ (deuterated methanol);
mg (milligrams);
MHz (megahertz);
min (minutes);
mL (millilitres);
mmol (millimoles);
MsCl (methanesulfonyl chloride);
n-BuLi (n-butyllithium);
NaH (sodium hydride);
NaHCO₃ (sodium bicarbonate);
NaOEt (sodium ethoxide);
NaOH (sodium hydroxide);
NaOMe (sodium methoxide);
Na₂SO₄ (sodium sulphate);
NBS (N-bromosuccinimide);
NIS (N-lodosuccinimide);
NH₄Cl (ammonium chloride);
NMP (N-methyl-2-pyrrolidone);
Pd/C (palladium on activated charcoal);
Pd₂(dba)₃ (tris(dibenzylideneacetone)dipalladium(0));
Pd(dppf)Cl₂ ([1,1′-Bis(diphenylphosphino)ferrocene]dichloropalladium(II));
Pd(dppf)Cl₂—CH₂Cl₂ ([1,1′-Bis(diphenylphosphino)ferrocene]dichloropalladium(II), complex with DCM);
Pd(OH)₂ (palladium(II) hydroxide on carbon—Pearlman's catalyst);
PE (Petroleum Ether)
prep-HPLC (preparative high-performance liquid chromatography);
prep-TLC (preparative thin layer chromatography);
ppm (parts per million);
psi (pounds per square inch);
p-TSA (p-toluenesulfonic acid);
quant. (quantitative yield)
RT (room temperature);
SEMCI (2-(trimethylsilyl)ethoxymethyl chloride);
SPhos (2-Dicyclohexylphosphino-2′,6′-dimethoxybiphenyl);
TBAF (tetra-n-butylammonium fluoride);
t-BuNHNH₂·HCl (tert-butylhydrazine hydrochloride);
TFA (trifluoroacetic acid);
THE (tetrahydrofuran);
TLC (thin layer chromatography);
v/v (volume/volume);
Xantphos (9,9-Dimethyl-4,5-bis(di-tert-butylphosphino)xanthene);

### LCMS Methodology

Electrospray mass spectroscopy (MS) was carried out using the following method.

Method A (5 minutes): LC model: Method A 1200 (Pump type: Binary Pump, Detector type: DAD) MS model: Method A G6110A Quadrupole. Column: Xbridge-C18, 2.5 μm, 2.1×30 mm. Column temperature: 30° C. Acquisition of wavelength: 214 nm, 254 nm. Mobile phase: A: 0.07% HCOOH aqueous solution, B: MeOH. Run time: 5 min. MS: Ion source: ES+(or ES−). MS range: 50-900 m/z. Fragmentor: 60. Drying gas flow: 10 L/min. Nebulizer pressure: 35 psi. Drying gas temperature: 350° C. Vcap: 3.5 kV.

Method B (3.5 minutes): LC model: Method A 1200 (Pump type: Binary Pump, Detector type: DAD) MS model: Method A G6110A Quadrupole. Column: Xbridge-C18, 2.5 μm, 2.1×30 mm. Column temperature: 30° C. Acquisition of wavelength: 214 nm, 254 nm. Mobile phase: A: 0.07% HCOOH aqueous solution, B: MeOH. Run time: 5 min. MS: Ion source: ES+(or ES−). MS range: 50-900 m/z. Fragmentor: 60. Drying gas flow: 10 L/min. Nebulizer pressure: 35 psi. Drying gas temperature: 350° C. Vcap: 3.5 kV.

Method C: (8 minutes) LC model: Waters 2695 alliance (Pump: Quaternary Pump, Detector: 2996 Photodiode Array Detector) MS model: Micromass ZQ LC: Column: Xbridge-C18, 3.5 μm, 2.1×50 mm Column temperature: 20° C. Acquisition of wavelength: 214 nm, 254 nm Mobile phase: A: 0.05% HCOOH aqueous solution, B: CAN Run time: 8 min MS: Ion source: ES+(or ES−) MS range: 100-1000 m/z Capillary: 3 kv Cone: 40 V Extractor: 3 V Drying gas flow: 800 L/hr cone: 50 L/hr Desolvation temperature: 500° C. Source temperature: 120° C.

Method D: Mass detector: Agilent G6120B MSD Pump: 1260 Infinity G1312B Binary pump Autosampler: 1260 Infinity G1367E HiPALS Detector: 1260 Infinity G4212B DAD Column: Poroshell 120 EC-C18, 2.1×30 mm 2.7 Micron Column temperature: 30° C. Injection volume: 2 μL Flowrate: 1.0 ml/min Solvent A: Water 0.1% Formic Acid Solvent B: Acetonitrile 0.1% Formic Acid Gradient: 5-100% B over 3.8 min Acquisition time: 4.1 min Detection: 254 nm and 254 nm Ion source: Single Quadrupole Ion Mode: API-ES Drying gas temperature: 350° C. Capillary voltage (V): 4000 (positive) Capillary voltage (V): 4000 (negative) Scan Range: 100-1000 Step size: 0.1 sec

Method E: Mass detector: Agilent G6120B MSD Pump: 1260 Infinity G1312B Binary pump Autosampler: 1260 Infinity G1367E HiPALS Detector: 1260 Infinity G4212B DAD Column: Atlantis T3, 3 μM, 100A, 3.0×50 mm Column temperature: 30° C. Injection volume: 1 μL Flowrate: 1.0 ml/min Solvent A: Water 0.1% Formic Acid Solvent B: Acetonitrile 0.1% Formic Acid Gradient: 5-50% B over 3.0 min Acquisition time: 4.1 min Detection: 214 and 254 nm Ion source: Single Quadrupole Ion Mode: API-ES Drying gas temperature: 350° C. Capillary voltage (V): 4000 (positive) Capillary voltage (V): 4000 (negative) Scan Range: 100-1000 Step size: 0.1 sec

Method F: Agilent: Mass detector: Agilent G6120B MSD, Pump: 1260 Infinity G1312B Binary pump, Autosampler: 1260 Infinity G1367E HiPALS, Detector: 1260 Infinity G4212B DAD. LC conditions: Column: Poroshell 120 EC-C18, 2.1×30 mm 2.7 Micron, Column temperature: 30° C. Injection volume: 1 uL, Flowrate: 1.0 ml/min, Solvent A: Water 0.1% Formic Acid, Solvent B: Acetonitrile 0.1% Formic Acid, Gradient: 5-100% B over 3.8 min, Acquisition time: 4.1 min, Detection: 214 and 254 nm. MS conditions: Ion source: Single Quadrupole, Ion Mode: API-ES, Drying gas temperature: 350° C., Capillary voltage (V): 4000 (positive), Capillary voltage (V): 4000 (negative), Scan Range: 100-1000, Step size: 0.1 sec

Method G: Agilent High MW: Mass detector: Agilent G6120B MSD, Pump: 1260 Infinity G1312B Binary pump, Autosampler: 1260 Infinity G1367E HiPALS, Detector: 1260 Infinity G4212B DAD. LC conditions: Column: Poroshell 120 EC-C18, 2.1×30 mm 2.7 Micron, Column temperature: 30° C., Injection volume: 1 uL, Flowrate: 1.0 ml/min, Solvent A: Water 0.1% Formic Acid, Solvent B: Acetonitrile 0.1% Formic Acid, Gradient: 5-100% B over 3.8 min, Acquisition time: 4.1 min, Detection: 214 and 254 nm. MS conditions: Ion source: Single Quadrupole, Ion Mode: API-ES, Drying gas temperature: 350° C., Capillary voltage (V): 4000 (positive), Capillary voltage (V): 4000 (negative), Scan Range: 100-2000, Step size: 0.1 sec

Method H: Waters: Waters ZQ 3100—Mass Detector, Waters 2545-Pump, Waters SFO System Fluidics Organizer, Waters 2996 Diode Array Detector, Waters 2767 Sample Manager. LC conditions: Reverse Phase HPLC analysis, Column: Xbridge™ C18 5 μm 4.6×100 mm, Injection Volume 10 μL, Solvent A: Water 0.1% Formic Acid, Solvent B: Acetonitrile 0.1% Formic Acid, Gradient: 10-100% B over 8 min, Flow rate: 1.5 ml/min, Detection: 100-600 nm. MS conditions: Ion Source: Single-quadrupole, Ion Mode: ES positive, Source Temp: 150° C., Desolvation Temp: 350° C., Detection: Ion counting, Capillary (KV)-3.00, Cone (V): 30, Extractor (V): 3, RF Lens (V): 0.1, Scan Range: 100-1000 Amu, Scan Time: 0.5 sec, Acquisition time: 10 min, Gas Flow: Desolvation L/hr-650, Cone L/hr-100

### Preparative HPLC

Method A: Instrument type: VARIAN 940 LC. Pump type: Binary Pump. Detector type: PDA. LC conditions: Column: Waters SunFire prep C18 OBD, 5 μm, 19×100 mm. Acquisition wavelength: 214 nm, 254 nm. Mobile Phase: A: 0.07% TFA aqueous solution (or 0.1% HCOOH aqueous solution), B: MeOH (or CH₃CN).

Method B: Waters ZQ 3100—Mass Detector, Waters 2545-Pump, Waters SFO System Fluidics Organizer, Waters 2996 Diode Array Detector, Waters 2767 Sample Manager. LC conditions: Column: Xbridge™ prep C18 OBD 5 μm 19×100 mm, Solvent A: Water 0.1% Formic Acid, Solvent B: Acetonitrile 0.1% Formic Acid, Gradient: variable, Flow rate: 20 ml/min, Detection: 100-600 nm MS conditions: Ion Source: Single-quadrupole, Ion Mode: ES positive, Source Temp: 150° C., Desolvation Temp: 350° C., Detection: Ion counting, Capillary (KV)-3.00, Cone (V): 30, Extractor (V):3, RF Lens (V): 0.1, Scan Range: 100-1000 Amu, Scan Time: 0.5 sec, Acquisition time: 20 min Gas Flow: Desolvation L/hr-650, Cone L/hr-100

Method C: Waters ZQ 3100—Mass Detector, Waters 2545-Pump, Waters SFO System Fluidics Organizer, Waters 2996 Diode Array Detector, Waters 2767 Sample Manager. LC conditions: Column: Xbridge™ prep C18 OBD 5 μm 19×100 mm, Solvent A: Water, Solvent B: Acetonitrile, Gradient: variable, Flow rate: 20 ml/min, Detection: 100-600 nm MS conditions: Ion Source: Single-quadrupole, Ion Mode: ES positive, Source Temp: 150° C., Desolvation Temp: 350° C., Detection: Ion counting, Capillary (KV)-3.00, Cone (V): 30, Extractor (V):3, RF Lens (V): 0.1, Scan Range: 100-1000 Amu, Scan Time: 0.5 sec, Acquisition time: 20 min Gas Flow: Desolvation L/hr-650, Cone L/hr-100

### NMR

Nuclear magnetic resonance spectra were recorded on a Bruker 400 MHz or 300 MHz for ¹H nuclei as specified. Samples were recorded in deuterated solvent as specified, and data acquired at 25° C. Chemical shifts are reported in ppm on the δ scale and referenced to the appropriate solvent peak. —In reporting spectral data, the following abbreviations have been used: s, singlet; bs, broad singlet; d, doublet; t, triplet; q, quartet; m, multiplet.

### Synthesis of Intermediates A

### Intermediate A1: 3-iodo-1-methyl-1H-pyrazolo[4,3-c]pyridin-4-amine

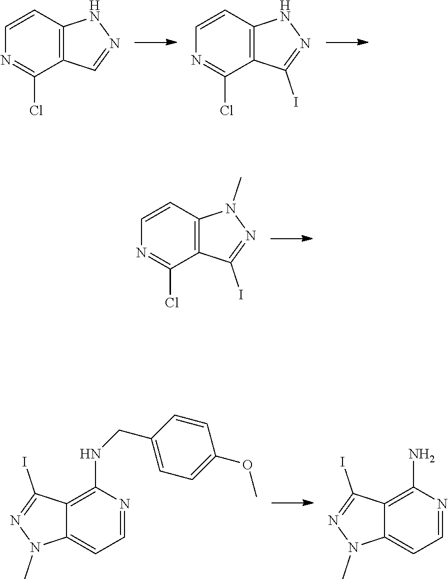 [SMILES: Clc1nccc2[nH]ncc12 ; Clc1nccc2[nH]nc(I)c12 ; Cn1nc(I)c2c(Cl)nccc21 ; COc1ccc(CNc2nccc3c2c(I)nn3C)cc1 ; Cn1nc(I)c2c(N)nccc21]

### Step 1: 4-chloro-3-iodo-1H-pyrazolo[4,3-c]pyridine

To a solution of 4-chloro-1H-pyrazolo[4,3-c]pyridine (25 g, 162 mmol) in DMF (200 mL) was added NIS (43.6 g, 194 mmol) and the mixture was stirred at 85° C. overnight. The mixture was diluted with water (2,500 mL) and the solids were filtered to give the title product (43.0 g, 94%) as a yellow solid. LCMS (Method A): 2.99 min, m/z: 279.9 [M+H]⁺.

### Step 2: 4-chloro-3-iodo-1-methyl-1H-pyrazolo[4,3-c]pyridine

To a pre-cooled solution of 4-chloro-3-iodo-1H-pyrazolo[4,3-c]pyridine (43 g, 153 mmol) in DMF (200 mL) at 0° C. was added NaH (60% in oil, 18.4 g, 765 mmol). The mixture was stirred at 0° C. under N₂ for 10 min. Iodomethane (32.5 g, 229 mmol) was then added slowly and the mixture and stirred at rt for 1 h. Water (2,000 mL) was added and the product was extracted with EtOAc (2×1000 mL). The combined organics were dried over MgSO₄, concentrated under reduced pressure and the residue was purified by column chromatography (DCM/PE=4/1, v/v) to give the title product (23.5 g, 52%) as a white solid. LCMS (Method A): 3.42 min, m/z: 293.8 [M+H]⁺.

### Step 3: 3-iodo-N-(4-methoxybenzyl)-1-methyl-1H-pyrazolo[4,3-c]pyridin-4-amine

To a solution of 4-chloro-3-iodo-1-methyl-1H-pyrazolo[4,3-c]pyridine (23.5 g, 80.0 mmol) in 1-butanol (200 mL) was added (4-methoxyphenyl)methanamine (44.0 g, 320 mmol) and the mixture was stirred at 110° C. overnight. The mixture was concentrated under reduced pressure before the addition of water (2,000 mL). The precipitate was filtered off to give the title product (33.0 g) as a white solid. LCMS (Method A): 2.58 min, m/z: 395.0 [M+H]⁺.

### Step 4: 3-iodo-1-methyl-1H-pyrazolo[4,3-c]pyridin-4-amine

A solution of 3-iodo-N-[(4-methoxyphenyl)methyl]-1-methyl-1H-pyrazolo[4,3-c]pyridin-4-amine (33 g, 83.7 mmol) in TFA (60 mL) was stirred at 70° C. overnight. The mixture was concentrated under reduced pressure and the residue was partitioned between an aqueous solution of Na₂CO₃ (250 mL) and DCM (250 mL). The organics were washed with water, dried over Na₂SO₄ and concentrated under reduced pressure to give the title product (16.4 g, 71% over 2 steps) as a white solid. LCMS (Method A): 0.91 min. m/z: 275.0 [M+H]⁺.

### Intermediate A2: 3-iodo-1H-pyrazolo[4,3-c]pyridin-4-amine

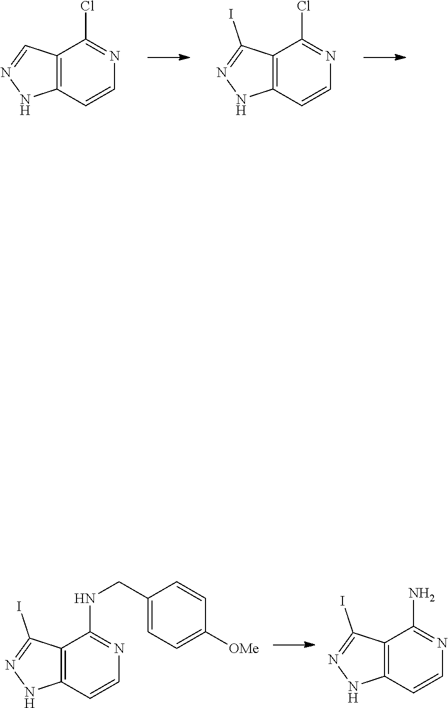 [SMILES: Clc1nccc2[nH]ncc12 ; Clc1nccc2[nH]nc(I)c12 ; COc1ccc(CNc2nccc3[nH]nc(I)c23)cc1 ; Nc1nccc2[nH]nc(I)c12]

### Step 1: 4-chloro-3-iodo-1H-pyrazolo[4,3-c]pyridine

To a solution of 4-chloro-1H-pyrazolo[4,3-c]pyridine (25 g, 162 mmol) in DMF (200 mL) was added NIS (43.6 g, 194 mmol) and the reaction was stirred at 85° C. overnight. The mixture was diluted with water (2500 mL) and the precipitate was collected via filtration to afford the title product (43.0 g, 95%) as a yellow solid. LCMS (method A): 2.99 min, m/z: 279.9 [M+H]⁺.

### Step 2: 3-iodo-N-[(4-methoxyphenyl)methyl]-1H-pyrazolo[4,3-c]pyridin-4-amine

A mixture of 4-chloro-3-iodo-1H-pyrazolo[4,3-c]pyridine (5 g, 17.8 mmol) and 1-(4-methoxyphenyl)methanamine (12.2 g, 89.0 mmol) in DMSO (100 mL) was stirred at 150° C. for 6 h. The mixture was poured into water (100 mL) and extracted with EtOAc (50 mL×2). The combined organic phases were dried (Na₂SO₄) and concentrated. The residue was purified by column chromatography (PE/EtOAc=3/1, v/v) to afford the title product (2.2 g, 33%) as a yellow solid. LCMS (method D): 0.70 min, m/z: 381.0 [M+H]⁺.

### Step 3: 3-iodo-1H-pyrazolo[4,3-c]pyridin-4-amine

A solution of 3-iodo-N-[(4-methoxyphenyl)methyl]-1H-pyrazolo[4,3-c]pyridin-4-amine (1.91 g, 5.02 mmol) in TFA (15 mL) was stirred at 75° C. overnight. The mixture was adjusted to pH=7-8 with sat. aq. Na₂CO₃ and extracted with DCM (50 mL×2). The organics were concentrated, and the residue was purified by Prep-TLC (DCM/MeOH=20/1, v/v), to afford the title product (600 mg, 46%) as a yellow solid. ¹H NMR (400 MHz, DMSO-d₆): 13.38 (bs, 1H), 7.60 (d, J=6.4 Hz, 1H), 6.69 (d, J=6.4 Hz, 1H), 6.17 (bs, 2H).

### Intermediate A3: 3-iodo-1-isopropyl-1H-pyrazolo[4,3-c]pyridin-4-amine

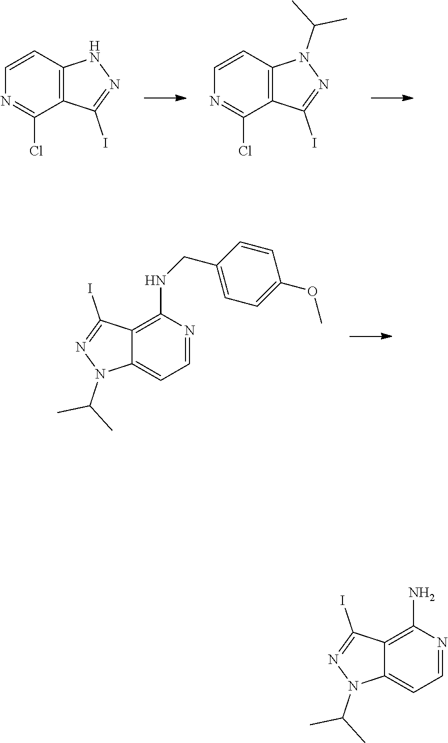

### Step 1: 4-chloro-3-iodo-1-isopropyl-1H-pyrazolo[4,3-c]pyridine

To a mixture of 4-chloro-3-iodo-1H-pyrazolo[4,3-c]pyridine (6 g, 21.4 mmol) and Cs₂CO₃ (20.8 g, 64.1 mmol) in acetonitrile (200 mL) was added 2-bromopropane (5.26 g, 42.8 mmol) and the reaction mixture was stirred at 70° C. overnight. Water (200 mL) was added, and the product was extracted with EtOAc (2×100 mL). The combined organics were dried over Na₂SO₄, concentrated under reduced pressure and the residue was purified by column chromatography (PE/EtOAc=4/1 to 1/4, v/v) to give the title product (4 g, 58%) as a yellow solid. LCMS (Method A): 2.60 min, m/z: 322.0 [M+H]⁺.

### Step 2: 3-iodo-1-isopropyl-N-(4-methoxybenzyl)-1H-pyrazolo[4,3-c]pyridin-4-amine

To a solution of 4-chloro-3-iodo-1-isopropyl-1H-pyrazolo[4,3-c]pyridine (4 g, 12.4 mmol) in 1-butanol (40 mL) was added (4-methoxyphenyl)methanamine (8.50 g, 62.0 mmol) and the mixture was stirred at 110° C. overnight. The reaction mixture was concentrated under reduced pressure and the residue partitioned between water (200 mL) and EtOAc (200 mL). The layers were separated, and the aqueous layer was further extracted with EtOAc (100 mL). The combined organics were washed with brine, dried over Na₂SO₄ and concentrated under reduced pressure to give the crude product (2 g, 38%) as a brown oil. LCMS (Method A): 2.95 min, m/z: 423.1 [M+H]⁺.

### Step 3: 3-iodo-1-isopropyl-1H-pyrazolo[4,3-c]pyridin-4-amine

A solution of 3-iodo-1-isopropyl-N-(4-methoxybenzyl)-1H-pyrazolo[4,3-c]pyridin-4-amine (3.7 g, 8.76 mmol) in TFA (20 mL) was stirred at 70° C. overnight. The mixture was concentrated under reduced pressure and the residue was partitioned between an aqueous solution of Na₂CO₃ (250 mL) and DCM (250 mL). The organics were washed with water, dried over Na₂SO₄ and concentrated under reduced pressure to give the title product (500 mg, 19%) as a yellow solid. LCMS (Method A): 1.83 min, m/z: 303.0 [M+H]⁺.

### Intermediate A4: 3-iodopyrazolo[1,5-a]pyrazin-4-amine

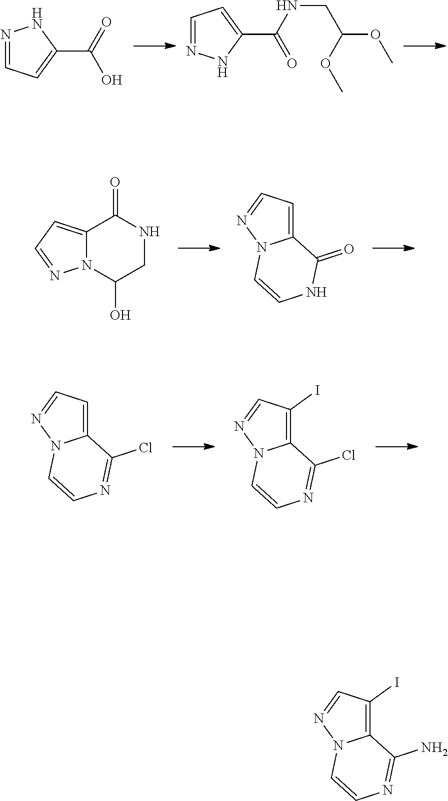

### Step 1: N-(2,2-dimethoxyethyl)-1H-pyrazole-5-carboxamide

A mixture of 1H-pyrazole-5-carboxylic acid (10 g, 89.2 mmol) and carbonyldiimidazole (15.9 g, 98.1 mmol) in 1,4-dioxane (100 ml) was stirred at 50° C. under N₂ for 1 hour before the addition of 2,2-dimethoxyethan-1-amine (10.3 g, 98.1 mmol), and the reaction mixture was stirred at 50° C. overnight. The reaction mixture was concentrated under reduced pressure and the residue was poured into water (500 mL). The aqueous solution was adjusted to pH=5 with 1 M HCl and the organics were extracted with EtOAc (2×250 mL). The combined organics were dried over Na₂SO₄ and concentrated under reduced pressure to give the title product (15 g, 84%) as a white solid. LCMS (Method A): 3.32 min; m/z: 222.1 [M+H]⁺.

### Step 2: 7-hydroxy-6,7-dihydropyrazolo[1,5-a]pyrazin-4(5H)-one

A solution of N-(2,2-dimethoxyethyl)-1H-pyrazole-5-carboxamide (15 g, 75.2 mmol) in DCM (100 ml) and TFA (100 ml) was stirred at RT overnight. The reaction mixture was concentrated under reduced pressure to give the title product (12 g, 100%) as a brown oil.

### Step 3: pyrazolo[1,5-a]pyrazin-4(5H)-one

A solution of 7-hydroxy-6,7-dihydropyrazolo[1,5-a]pyrazin-4(5H)-one (12 g, 78.3 mmol) in polyphosphoric acid (50 mL) was stirred at 145° C. for 5 hours. The reaction mixture was adjusted to pH=10 with an aqueous NaOH solution. The mixture was concentrated under reduced pressure and the residue was purified by column chromatography (DCM/MeOH=20/1) to give the title product (6.1 g, 58%) as a brown solid. ¹H NMR (400 MHz, DMSO-d₆): 11.23 (s, 1H), 7.89 (s, 1H), 7.68 (d, J=5.8 Hz, 1H), 7.00 (s, 1H), 6.86 (d, J=11.5 Hz, 1H).

### Step 4: 4-chloropyrazolo[1,5-a]pyrazine

A solution of pyrazolo[1,5-a]pyrazin-4(5H)-one (6.1 g, 45.1 mmol) in POCl₃ (50 mL) and DMF (4 drops) was stirred at 130° C. overnight. The reaction mixture was slowly poured into ice water (300 mL), then adjusted to pH=10 with an aqueous NaOH solution. The organics were extracted with EtOAc (2×100 mL) and the combined organics were dried over Na₂SO₄ and concentrated under reduced pressure. The residue was purified by column chromatography (PE/EtOAc=10/1) to give the title product (3.6 g, 85%) as a white solid. ¹H NMR (400 MHz, DMSO-d₆): 8.86 (d, J=5.6 Hz, 1H), 8.26 (s, 1H), 7.75 (d, J=4.7 Hz, 1H), 7.05 (d, J=0.9 Hz, 1H).

### Step 5: 4-chloro-3-iodopyrazolo[1,5-a]pyrazine

To a solution of 4-chloropyrazolo[1,5-a]pyrazine (3.6 g, 23.4 mmol) in DMF (100 mL) was added NIS (10.5 g, 46.8 mmol) and the reaction was stirred at 85° C. overnight. The reaction mixture was poured into water (100 mL) and the solids were filtered, washed with water and dried under reduced pressure to give the title product (4.9 g, 75%) as a white solid. ¹H NMR (400 MHz, DMSO-d₆): 8.93 (d, J=4.7 Hz, 1H), 8.37 (s, 1H), 7.75 (d, J=4.7 Hz, 1H).

### Step 6: 3-iodopyrazolo[1,5-a]pyrazin-4-amine

To a solution of 4-chloro-3-iodopyrazolo[1,5-a]pyrazine (4.9 g, 17.5 mmol) in MeOH (50 mL) was added a solution of ammonia in MeOH (7 M, 100 mL) and the mixture was stirred at 110° C. in a sealed tube overnight. The mixture was concentrated under reduced pressure and the residue was purified by column chromatography (DCM/MeOH=50/1, v/v) to give the title product (3.00 g, 11.5 mmol) as a white solid. ¹H NMR (400 MHz, DMSO-d₆): 8.08 (d, J=4.7 Hz, 1H), 8.02 (d, J=11.8 Hz, 1H), 7.26 (t, J=4.7 Hz, 1H), 6.68 (d, J=37.2 Hz, 2H).

### Synthesis of Intermediates B

### Intermediate B1: (S)-1,1-difluoro-N-(2-(1-(4-fluorophenyl)ethoxy)-4-(4,4,5,5-tetramethyl-1,3,2-dioxaborolan-2-yl)phenyl)methanesulfonamide

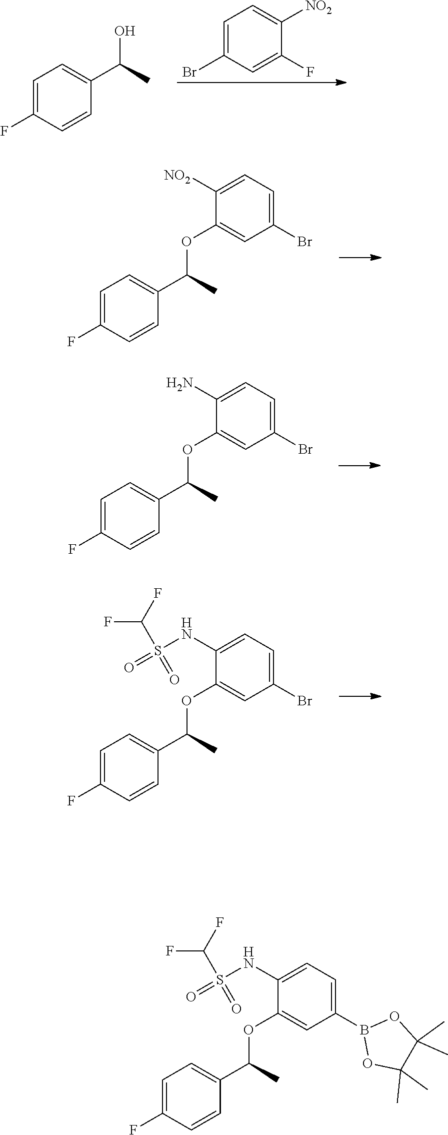

### Step 1: (S)-4-bromo-2-(1-(4-fluorophenyl)ethoxy)-1-nitrobenzene

To a solution of (1S)-1-(4-fluorophenyl)ethan-1-ol (40 g, 285 mmol) in THE (600 mL) was added NaH (60% in oil, 57.1 g, 1425 mmol) and the mixture was stirred at 0° C. under N₂ for 30 min. 4-Bromo-2-fluoro-1-nitrobenzene (62.6 g, 285 mmol) was then added, and the mixture was stirred at RT overnight. The reaction mixture was diluted with water (500 mL) and extracted with EtOAc (3×500 mL). The combined organics were washed with water and brine, dried (Na₂SO₄) and concentrated under reduced pressure to give the crude product (70 g, 72%) as a yellow oil. ¹H NMR (400 MHz, DMSO-d₆): δ 7.80 (d, J=8.4 Hz, 1H), 7.54 (d, J=2.0 Hz, 1H), 7.48 (q, J=4.8 Hz, 2H), 7.26 (dd, J=8.8, 2.0 Hz, 1H), 7.20 (t, J=8.8 Hz, 1H), 5.89 (q, J=6.4 Hz, 1H), 1.54 (d, J=6.0 Hz, 3H).

### Step 2: 4-bromo-2-[(1S)-1-(4-fluorophenyl)ethoxy]aniline

To a solution of 4-bromo-2-[(1S)-1-(4-fluorophenyl)ethoxy]-1-nitrobenzene (70 g, 205 mmol) in MeOH (500 mL), was added Zn dust (67.0 g, 1025 mmol) followed by sat. aq. NH₄Cl (170 mL). The mixture was stirred at 60° C. for 6 h, then diluted with water (500 mL) and extracted with EtOAc (3×500 mL). The combined organics were washed with water and brine, dried (Na₂SO₄) and concentrated under reduced pressure. The residue was purified by column chromatography (PE/EtOAc=100/1, v/v) to give the title product (40 g, 63%) as a brown oil. LCMS (method A): 4.13 min. m/z: 311.0, 312.0 [M+H]⁺.

### Step 3: N-{4-bromo-2-[(1S)-1-(4-fluorophenyl)ethoxy]phenyl}-1,1-difluoromethanesulfonamide

To a solution of 4-bromo-2-[(1S)-1-(4-fluorophenyl)ethoxy]aniline (40 g, 128 mmol) in DCM (200 mL) and pyridine (40.5 g, 512 mmol) was added difluoromethanesulfonyl chloride (24.9 g, 166 mmol). After stirring at RT overnight, the residue was diluted with water (500 mL) and extracted with DCM (3×500 mL). The combined organics were washed with brine, dried (Na₂SO₄) and concentrated under reduced pressure. The residue was purified by column chromatography (PE/EtOAc=100/1) to give the title product (39 g, 72%) as a brown oil. ¹H NMR (400 MHz, DMSO-d₆): 10.43 (s, 1H), 7.57 (q, J=4.8 Hz, 2H), 7.21-7.15 (m, 3H), 7.07-7.04 (m, 2H), 6.98 (t, J=52.4 Hz, 1H), 5.65 (q, J=6.4 Hz, 1H), 1.54 (d, J=6.0 Hz, 3H).

### Step 4: (S)-1,1-difluoro-N-(2-(1-(4-fluorophenyl)ethoxy)-4-(4,4,5,5-tetramethyl-1,3,2-dioxaborolan-2-yl)phenyl)methanesulfonamide

To a solution of N-{4-bromo-2-[(1S)-1-(4-fluorophenyl)ethoxy]phenyl}-1,1-difluoromethanesulfonamide (39 g, 91.9 mmol) in dioxane (300 mL), was added KOAc (26.9 g, 275 mmol), B₂pin₂ (34.7 g, 137 mmol) and Pd(dppf)Cl₂ (2.01 g, 2.75 mmol). The mixture was stirred at 100° C. under N₂ overnight, then concentrated under reduced pressure. The residue was diluted with water (500 mL) and extracted with EtOAc (3×500 mL). The combined organics were washed with water and brine, dried (Na₂SO₄) and concentrated under reduced pressure. The residue was purified by column chromatography (PE/EtOAc=50/1) to give the desired product (41 g, 94%) as a yellow oil. ¹H NMR (400 MHz, DMSO-d₆): 10.41 (s, 1H), 7.58 (q, J=4.8 Hz, 2H), 7.27 (d, J=7.6 Hz, 1H), 7.20-7.16 (m, 3H), 7.12 (s, 1H), 6.98 (t, J=52.4 Hz, 1H), 5.63 (q, J=6.4 Hz, 1H), 1.54 (d, J=6.4 Hz, 3H), 1.25 (d, J=4.8 Hz, 12H). The following intermediates B were similarly prepared from the appropriate benzyl alcohol (step 1) according to the preparation of (S)-1,1-difluoro-N-(2-(1-(4-fluorophenyl)ethoxy)-4-(4,4,5,5-tetramethyl-1,3,2-dioxaborolan-2-yl)phenyl)methanesulfonamide (intermediate B1)

TABLE 1
Intermediates B2 to B16

| Intermediate | Name | Structure | SM |
|---|---|---|---|
| B2 | (R)-1,1-difluoro-N-(2-(1-(4- fluorophenyl)ethoxy)-4-(4,4,5,5- tetramethyl-1,3,2-dioxaborolan-2- yl)phenyl)methanesulfonamide | 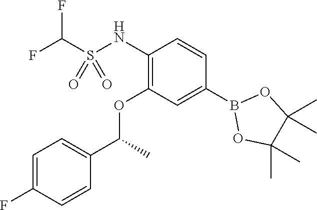 [SMILES: C[C@@H](Oc1cc(B2OC(C)(C)C(C)(C)O2)ccc1NS(=O)(=O)C(F)F)c1ccc(F)cc1] | (1R)-1-(4- fluorophenyl)ethan- 1-ol |
| B3 | (S)-N-(2-(1-(3- chlorophenyl)ethoxy)-4-(4,4,5,5- tetramethyl-1,3,2-dioxaborolan-2- yl)phenyl)-1,1- difluoromethanesulfonamide | 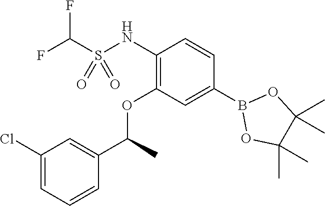 [SMILES: C[C@H](Oc1cc(B2OC(C)(C)C(C)(C)O2)ccc1NS(=O)(=O)C(F)F)c1cccc(Cl)c1] | (S)-1-(3- chlorophenyl)ethan- 1-ol |
| B4 | (S)-1,1-difluoro-N-(2-(1-(2- fluorophenyl)ethoxy)-4-(4,4,5,5- tetramethyl-1,3,2-dioxaborolan-2- yl)phenyl)methanesulfonamide | 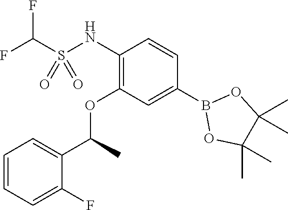 [SMILES: C[C@H](Oc1cc(B2OC(C)(C)C(C)(C)O2)ccc1NS(=O)(=O)C(F)F)c1ccccc1F] | (1S)-1-(2- fluorophenyl)ethan- 1-ol |
| B5 | 1,1-difluoro-N-(2-(1-(4- fluorophenyl)-2-methoxyethoxy)-4- (4,4,5,5-tetramethyl-1,3,2- dioxaborolan-2- yl)phenyl)methanesulfonamide | 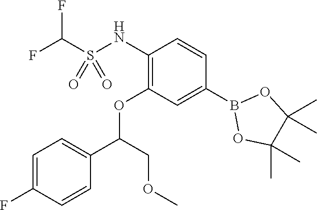 [SMILES: COCC(Oc1cc(B2OC(C)(C)C(C)(C)O2)ccc1NS(=O)(=O)C(F)F)c1ccc(F)cc1] | 1-(4-fluorophenyl)-2- methoxyethan-1-ol |
| B6 | N-(2-((4-chloro-3-fluorobenzyl)oxy)- 4-(4,4,5,5-tetramethyl-1,3,2- dioxaborolan-2-yl)phenyl)-1,1- difluoromethanesulfonamide | 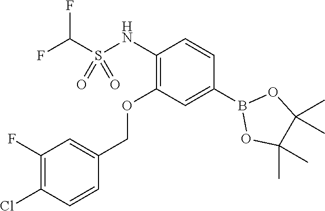 [SMILES: CC1(C)OB(c2ccc(NS(=O)(=O)C(F)F)c(OCc3ccc(Cl)c(F)c3)c2)OC1(C)C] | (4-chloro-3- fluorophenyl)methanol |
| B7 | (S)-1,1-difluoro-N-(2-(1-(4- methoxyphenyl)ethoxy)-4-(4,4,5,5- tetramethyl-1,3,2-dioxaborolan-2- yl)phenyl)methanesulfonamide | 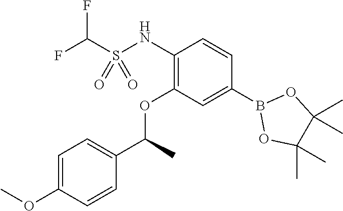 | (S)-1-(4- methoxyphenyl)ethan- 1-ol |
| B8 | N-(2-((3-chlorobenzyl)oxy)-4- (4,4,5,5-tetramethyl-1,3,2- dioxaborolan-2-yl)phenyl)-1,1- difluoromethanesulfonamide | 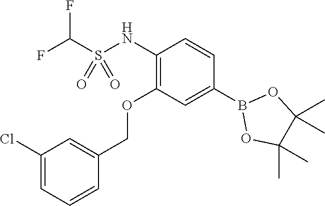 [SMILES: CC1(C)OB(c2ccc(NS(=O)(=O)C(F)F)c(OCc3cccc(Cl)c3)c2)OC1(C)C] | (3-chlorophenyl) methanol |
| B9 | N-(2-((6-chloropyridin-3- yl)methoxy)-4-(4,4,5,5-tetramethyl- 1,3,2-dioxaborolan-2-yl)phenyl)-1,1- difluoromethanesulfonamide | 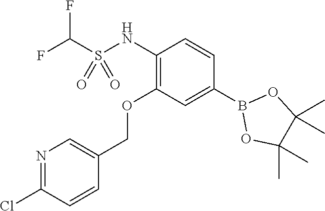 [SMILES: CC1(C)OB(c2ccc(NS(=O)(=O)C(F)F)c(OCc3ccc(Cl)nc3)c2)OC1(C)C] | (6-chloropyridin-3- yl)methanol |
| B10 | 1,1-difluoro-N-(2-(1-(4- fluorophenyl)cyclopropoxy)-4- (4,4,5,5-tetramethyl-1,3,2- dioxaborolan-2- yl)phenyl)methanesulfonamide | 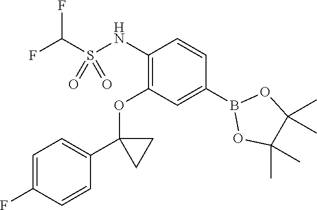 [SMILES: CC1(C)OB(c2ccc(NS(=O)(=O)C(F)F)c(OC3(c4ccc(F)cc4)CC3)c2)OC1(C)C] | 1-(4-fluorophenyl) cyclopropan-1-ol |
| B11 | 1,1-difluoro-N-(4-(4,4,5,5- tetramethyl-1,3,2-dioxaborolan-2- yl)-2-(thiazol-2- ylmethoxy)phenyl)methanesulfona mide | 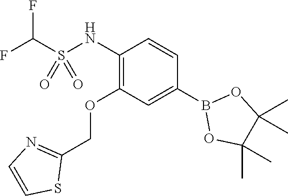 [SMILES: CC1(C)OB(c2ccc(NS(=O)(=O)C(F)F)c(OCc3nccs3)c2)OC1(C)C] | thiazol-2-ylmethanol |
| B12 | 1,1-difluoro-N-(2-isopropoxy-4- (4,4,5,5-tetramethyl-1,3,2- dioxaborolan-2- yl)phenyl)methanesulfonamide | 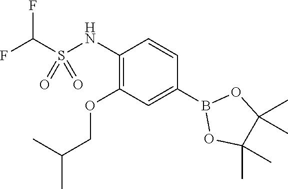 [SMILES: CC(C)COc1cc(B2OC(C)(C)C(C)(C)O2)ccc1NS(=O)(=O)C(F)F] | isopropanol |
| B13 | N-(2-(cyclohexylmethoxy)-4- (4,4,5,5-tetramethyl-1,3,2- dioxaborolan-2-yl)phenyl)-1, 1- difluoromethanesulfonamide | 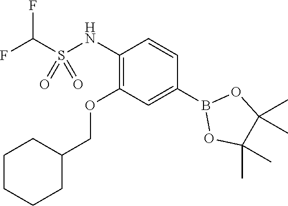 [SMILES: CC1(C)OB(c2ccc(NS(=O)(=O)C(F)F)c(OCC3CCCCC3)c2)OC1(C)C] | cyclohexylmethanol |
| B14 | 1,1-difluoro-N-(2-(oxazol-2- ylmethoxy)-4-(4,4,5,5-tetramethyl- 1,3,2-dioxaborolan-2- yl)phenyl)methanesulfonamide | 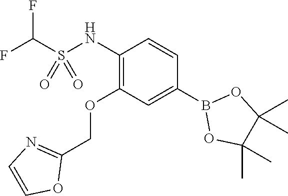 [SMILES: CC1(C)OB(c2ccc(NS(=O)(=O)C(F)F)c(OCc3ncco3)c2)OC1(C)C] | oxazol-2-ylmethanol |
| B15 | (S)-N-(2-(1-(4- chlorophenyl)ethoxy)-4-(4,4,5,5- tetramethyl-1,3,2-dioxaborolan-2- yl)phenyl)-1,1- difluoromethanesulfonamide | 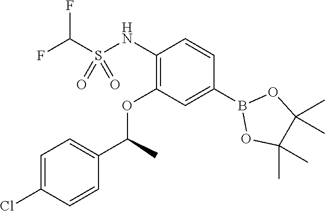 [SMILES: C[C@H](Oc1cc(B2OC(C)(C)C(C)(C)O2)ccc1NS(=O)(=O)C(F)F)c1ccc(Cl)cc1] | (S)-1-(4- chlorophenyl)ethan- 1-ol |
| B16 | N-(2-(benzo[d][1,3]dioxol-5- ylmethoxy)-4-(4,4,5,5-tetramethyl- 1,3,2-dioxaborolan-2-yl)phenyl)-1,1- difluoromethanesulfonamide | 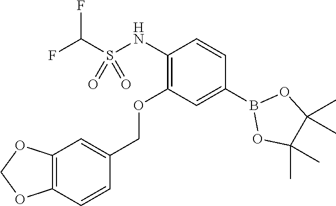 [SMILES: CC1(C)OB(c2ccc(NS(=O)(=O)C(F)F)c(OCc3ccc4c(c3)OCO4)c2)OC1(C)C] | benzo[d][1,3]dioxol- 5-ylmethanol |

### Intermediate B17: N-[4-(4,4,5,5-tetramethyl-1,3,2-dioxaborolan-2-yl)phenyl]ethane-1-sulfonamide

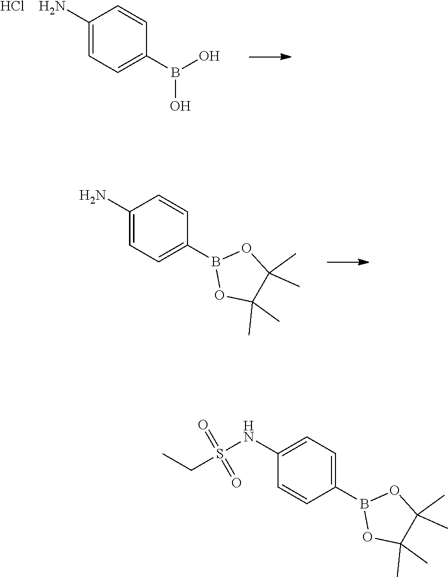 [SMILES: Cl ; Nc1ccc(B(O)O)cc1 ; CC1(C)OB(c2ccc(N)cc2)OC1(C)C ; CCS(=O)(=O)Nc1ccc(B2OC(C)(C)C(C)(C)O2)cc1]

### Step 1: 4-(4,4,5,5-tetramethyl-1,3,2-dioxaborolan-2-yl)aniline

A mixture of (4-aminophenyl)boronic acid hydrochloride (2 g, 11.5 mmol), pinacol (1.48 g, 12.6 mmol), MgSO₄ (4.15 g, 34.5 mmol) and NaHCO₃ (1.93 g, 23.0 mmol) in anhydrous THE (11.5 mL) was stirred under N₂ at RT overnight. The mixture was diluted with EtOAc, filtered over Celite and concentrated to give an off-white solid. The solid was triturated with Et₂O, collected via filtration, washed with Et₂O and air-dried to afford the title compound (1.49 g, 59%) as a white solid. LCMS (method F): 1.44 min, m/z: 220.4 [M+H]⁺.

### Step 2: N-[4-(4,4,5,5-tetramethyl-1,3,2-dioxaborolan-2-yl)phenyl]ethane-1-sulfonamide

To a solution of 4-(4,4,5,5-tetramethyl-1,3,2-dioxaborolan-2-yl)aniline (1.49 g, 6.80 mmol) in anhydrous DCM (13.6 mL) were added pyridine (2.74 mL, 33.9 mmol) and EtSO₂Cl (1.28 mL, 13.6 mmol), and the reaction was stirred at RT under N₂ for 4 h. The mixture was concentrated, and the residue was diluted with H₂O (50 mL) and extracted with EtOAc (3×30 mL). The combined organics were dried over MgSO₄ and concentrated. The residue was purified by flash chromatography (0-50% EtOAc:c-Hex) to afford the title compound (1.88 g, 89%) as a white solid. LCMS (method F): 1.98 min, m/z: 312.2 [M+H]⁺. ¹H NMR (CDCl₃): 7.77 (d, J=8.5 Hz, 2H), 7.19 (d, J=8.5 Hz, 2H), 6.72 (s, 1H), 3.15 (q, J=7.4 Hz, 2H), 1.33 (t, J=7.4 Hz, 3H).

### Intermediate B18: 1,1-difluoro-N-[4-(4,4,5,5-tetramethyl-1,3,2-dioxaborolan-2-yl)phenyl]methanesulfonamide

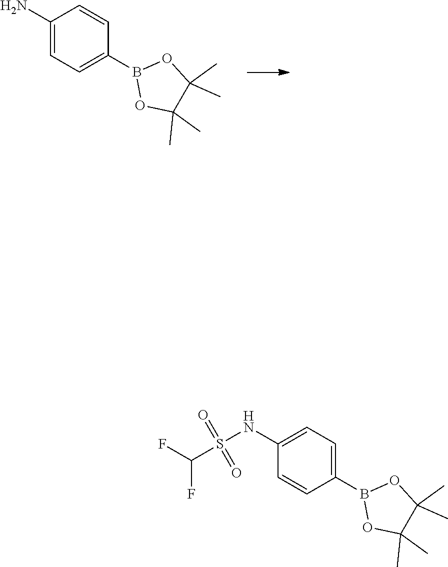 [SMILES: CC1(C)OB(c2ccc(N)cc2)OC1(C)C ; CC1(C)OB(c2ccc(NS(=O)(=O)C(F)F)cc2)OC1(C)C]

### Step 1: 1,1-difluoro-N-[4-(4,4,5,5-tetramethyl-1,3,2-dioxaborolan-2-yl)phenyl]methanesulfonamide

To a solution of 4-(4,4,5,5-tetramethyl-1,3,2-dioxaborolan-2-yl)aniline (500 mg, 2.28 mmol) in DCM (8 mL) were added pyridine (921 μL, 11.4 mmol) and difluoromethanesulfonyl chloride (241 μL, 2.73 mmol), and the reaction was stirred at RT under N₂. After 1 h, the mixture was concentrated and then azeotroped with PhMe (10 mL). The residue was purified by flash chromatography (0-25% EtOAc/heptane) to afford the title compound (555 mg, 73%) as a white solid. LCMS (Method F): 2.47 min, m/z: 334.2 [M+H]⁺. ¹H NMR (300 MHz, CDCl₃): 7.81-7.79 (m, 2H), 7.27-7.24 (m, 2H), 6.71 (br. s, 1H), 6.25 (t, J=53.5 Hz, 1H), 1.33 (s, 12H).

### Intermediates B19: N-(2-((4-fluorobenzyl)oxy)-4-(4,4,5,5-tetramethyl-1,3,2-dioxaborolan-2-yl)phenyl)ethanesulfonamide

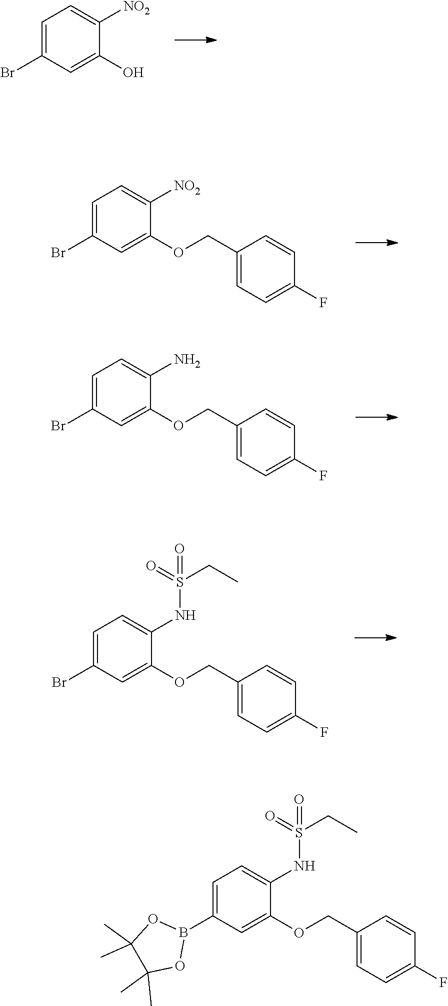 [SMILES: O=[N+]([O-])c1ccc(Br)cc1O ; O=[N+]([O-])c1ccc(Br)cc1OCc1ccc(F)cc1 ; Nc1ccc(Br)cc1OCc1ccc(F)cc1 ; CCS(=O)(=O)Nc1ccc(Br)cc1OCc1ccc(F)cc1 ; CCS(=O)(=O)Nc1ccc(B2OC(C)(C)C(C)(C)O2)cc1OCc1ccc(F)cc1]

### Step 1: 4-bromo-2-((4-fluorobenzyl)oxy)-1-nitrobenzene

A mixture of 5-bromo-2-nitrophenol (10 g, 45.8 mmol), K₂CO₃ (12.6 g, 91.6 mmol) and 1-(bromomethyl)-4-fluorobenzene (8.65 g, 45.8 mmol) in MeCN (100 mL) was stirred at 70° C. under N₂ for 16 h. The mixture was diluted with H₂O (100 mL) extracted with EtOAc (3×200 mL). The combined organic layers were dried (Na₂SO₄) and then concentrated under reduced pressure to afford the title product (15.0 g, 100%) as a white solid.

### Step 2: 4-bromo-2-((4-fluorobenzyl)oxy)aniline

To a solution of 4-bromo-2-((4-fluorobenzyl)oxy)-1-nitrobenzene (15 g, 45.9 mmol) in MeOH (300 mL) and sat. aq. NH₄Cl (100 mL), was added Zn dust (14.9 g, 229 mmol) and the reaction mixture was stirred at 60° C. for 4 h. The mixture was filtered, and the filtrate was concentrated under reduced pressure. The residue was partitioned between H₂O (250 mL) and EtOAc (300 mL) and the organic layer was separated, dried (Na₂SO₄) and then concentrated under reduced pressure to afford the title product (13.0 g, 96%) as a black oil. LCMS (method A): 4.24 min; m/z: 296.0 [M+H]⁺.

### Step 3: N-(4-bromo-2-((4-fluorobenzyl)oxy)phenyl)ethanesulfonamide

A mixture of 4-bromo-2-((4-fluorobenzyl)oxy)aniline (13 g, 43.8 mmol), EtSO₂Cl (8.43 g, 65.6 mmol), pyridine (50 mL) and CHCl₃ (50 mL) was stirred at RT for 3 h. The mixture was concentrated under reduced pressure and the crude residue was purified by silica gel column chromatography (PE:EtOAc, 1:1) to afford the title product (12.5 g, 73%) as a yellow solid. LCMS (method A): 4.24 min; m/z: 410.0 [M+H]+.

### Step 4: N-(2-((4-fluorobenzyl)oxy)-4-(4,4,5,5-tetramethyl-1,3,2-dioxaborolan-2-yl)phenyl)ethanesulfonamide

A mixture of N-(4-bromo-2-((4-fluorobenzyl)oxy)phenyl)ethanesulfonamide (12.5 g, 32.1 mmol) Pd(dppf)Cl₂ (1.46 g, 1.60 mmol), AcOK (6.29 g, 64.2 mmol) and B₂pin₂ (8.96 g, 35.2 mmol) in degassed 1,4-dioxane (200 mL) was stirred at 100° C. under N₂ for 16 h. The mixture was concentrated under reduced pressure and the crude residue was purified by silica gel column chromatography (PE:EtOAc, 2:1) to afford the title product (14.8 g, >100%) as a brown solid. LCMS (method A): 4.51 min; m/z: 453.2 [M+H]⁺. ¹H NMR (400 MHz, DMSO-d₆): 8.99 (s, 1H), 7.63-7.60 (m, 2H), 7.38-7.32 (m, 2H), 7.28-7.21 (m, 3H), 5.15 (s, 2H), 3.03 (q, J=14.8, 7.2 Hz, 2H), 1.29 (s, 12H), 1.11 (t, J=7.2 Hz, 3H).

### Intermediate B20: N-[2-fluoro-4-(4,4,5,5-tetramethyl-1,3,2-dioxaborolan-2-yl)phenyl]ethane-1-sulfonamide

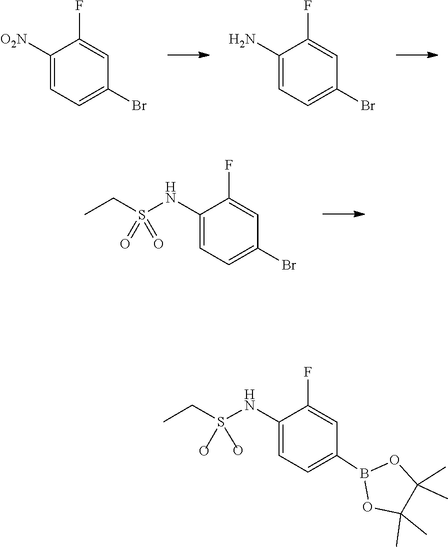

### Step 1: 4-bromo-2-fluoroaniline

To a solution of 4-bromo-2-fluoro-1-nitrobenzene (5 g, 22.7 mmol) in MeOH (12 mL) and sat. aq. NH₄Cl (4 mL) was added Zn (7.39 g, 113 mmol), And the reaction was stirred at 60° C. for 6 h. The mixture was concentrated, and the residue was diluted with water (150 mL) and extracted with EtOAc (150 mL×3). The combined organic phases were washed with water and brine, dried (Na₂SO₄) and concentrated. The residue was purified by column chromatography to afford the title product (2.5 g, 58%) as a brown oil. LCMS (method A): 3.39 min, m/z: 189.9, 191.9[M+H]⁺.

### Step 2: N-(4-bromo-2-fluorophenyl)ethane-1-sulfonamide

To a solution of 4-bromo-2-fluoroaniline (5 g, 26.3 mmol) in pyridine (20 mL) and DCM (80 mL) was added EtSO₂Cl (3.38 g, 26.3 mmol), and the reaction was stirred at RT overnight. The mixture was diluted with water and extracted with EtOAc (×3). The combined organics were dried (Na₂SO₄) and concentrated. The residue was purified by column chromatography (DCM/MeOH=20/1, v/v) to afford the title product (6.67 g, 90%) as a white solid. ¹H NMR (400 MHz, DMSO-d₆): 9.77 (s, 1H), 9.63 (dd, J=10.0, 2.0 Hz, 1H), 7.39-7.35 (m, 2H), 3.11 (q, J=7.6 Hz, 2H), 1.24, (t, J=7.6 Hz, 3H).

### Step 3: N-[2-fluoro-4-(4,4,5,5-tetramethyl-1,3,2-dioxaborolan-2-yl)phenyl]ethane-1-sulfonamide

A mixture of N-(4-bromo-2-fluorophenyl)ethane-1-sulfonamide (1.1 g, 3.89 mmol), B₂(pin)₂ (987 mg, 3.89 mmol), AcOK (763 mg, 7.78 mmol) and Pd(dppf)Cl₂ (317 mg, 389 μmol) in dioxane (10 mL) was stirred at 100° C. under N₂ overnight. The mixture was concentrated, and the residue was diluted with water (200 mL) and extracted with DCM (150 mL×3). The combined organic layers were washed with brine (200 mL), dried (Na₂SO₄), and concentrated. The residue was purified by prep-TLC (DCM/MeOH=20/1, v/v) to afford the title compound (1 g, 78%) as a black oil. ¹H NMR (400 MHz, DMSO-d₆): 9.87 (bs, 1H), 7.45-7.35 (m, 3H), 3.11 (q, J=7.6 Hz, 2H), 1.28 (s, 12H), 1.22, (t, J=7.6 Hz, 3H).

### Intermediate B21: 1,1-difluoro-N-{2-[(4-fluorophenyl)methoxy]-4-(4,4,5,5-tetramethyl-1,3,2-dioxaborolan-2-yl)phenyl}methanesulfonamide

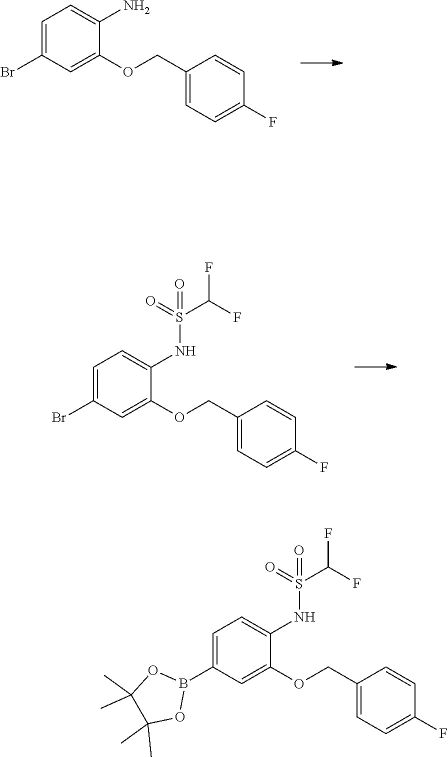 [SMILES: Nc1ccc(Br)cc1OCc1ccc(F)cc1 ; O=S(=O)(Nc1ccc(Br)cc1OCc1ccc(F)cc1)C(F)F ; CC1(C)OB(c2ccc(NS(=O)(=O)C(F)F)c(OCc3ccc(F)cc3)c2)OC1(C)C]

### Step 1: N-{4-bromo-2-[(4-fluorophenyl)methoxy]phenyl)-1,1-difluoromethanesulfonamide

To a solution of 4-bromo-2-[(4-fluorophenyl)methoxy]aniline (8.2 g, 27.6 mmol) in DCM (10 mL) were added pyridine (6.54 g, 82.8 mmol) and difluoromethanesulfonyl chloride (4.98 g, 33.1 mmol), and the resulting mixture was stirred at RT overnight. The reaction mixture was concentrated under reduced pressure and the residue was purified by column chromatography (PE/EtOAc=30/1) to give the title product (7.77 g, 69%) as a white solid.

### Step 2: 1,1-difluoro-N-{2-[(4-fluorophenyl)methoxy]-4-(4,4,5,5-tetramethyl-1,3,2-dioxaborolan-2-yl)phenyl}methanesulfonamide

A mixture of N-{4-bromo-2-[(4-fluorophenyl)methoxy]phenyl}-1,1-difluoromethanesulfonamide (7.77 g, 18.9 mmol), B₂(pin)₂ (5.25 g, 20.7 mmol), AcOK (3.70 g, 37.8 mmol) and Pd(dppf)Cl₂ (1.38 g, 1.89 mmol) in degassed 1,4-dioxane (100 mL) was stirred at 100° C. overnight under N₂. The reaction mixture was concentrated under reduced pressure and the residue was purified by silica gel column chromatography (PE/EtOAc=10/1) to give the title product (7.14 g, 82%) as a white solid.

### Synthesis of Intermediates AB

### Intermediate A1B1: N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide

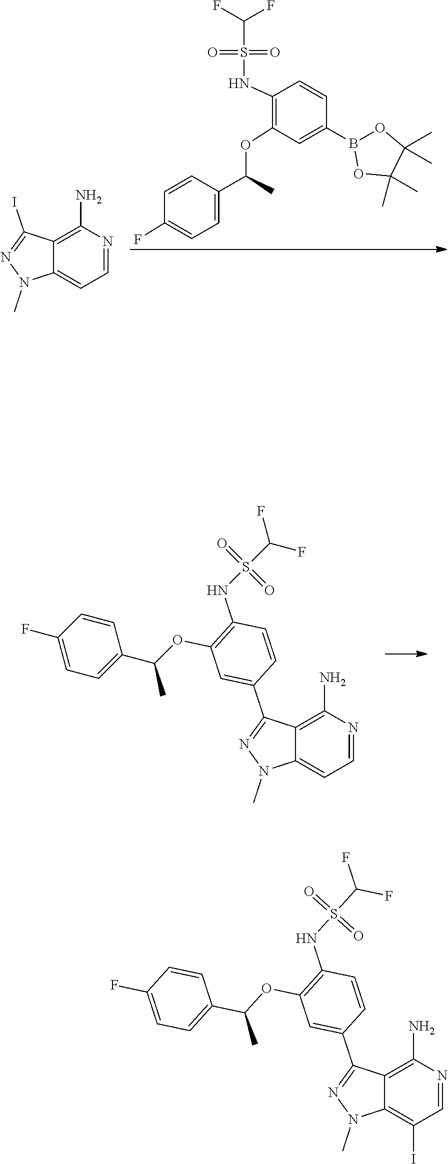

### Step 1: N-(4-{4-amino-1-methyl-1H-pyrazolo[4,3-c]pyridin-3-yl}-2-[(1S)-1-(4-fluorophenyl) ethoxy]phenyl)-1,1-difluoromethanesulfonamide (Compound 17)

To a solution of 3-iodo-1-methyl-1H-pyrazolo[4,3-c]pyridin-4-amine (8 g, 29.1 mmol) in degassed 1,4-dioxane and H₂O (4/1, 150 mL) was added 1,1-difluoro-N-{2-[(1S)-1-(4-fluorophenyl)ethoxy]-4-(4,4,5,5-tetramethyl-1,3,2-dioxaborolan-2-yl)phenyl}methanesulfonamide (15.0 g, 32.0 mmol), Na₂CO₃ (6.16 g, 58.2 mmol) and Pd(dppf)Cl₂ (1.18 g, 1.45 mmol) and the mixture was stirred at 100° C. overnight. The mixture was cooled to RT and concentrated under reduced pressure. The residue was purified by column chromatography (DCM/MeOH=40/1) to give the title product (12.0 g, 84%) as a yellow solid. LCMS (Method A): 2.92 min. m/z: 492.2 [M+H]⁺.

### Step 2: N-(4-{4-amino-7-iodo-1-methyl-1H-pyrazolo[4,3-c]pyridin-3-yl}-2-[(1 S)-1-(4-fluorophenyl)ethoxy]phenyl)-1,1-difluoromethanesulfonamide

To a solution of N-(4-{4-amino-1-methyl-1H-pyrazolo[4,3-c]pyridin-3-yl}-2-[(1S)-1-(4-fluorophenyl)ethoxy]phenyl)-1,1-difluoromethanesulfonamide (12 g, 24.4 mmol) in DMF (150 mL) was added NIS (6.57 g, 29.2 mmol) and the mixture was stirred at 85° C. overnight. Water (1500 mL) was added and the solids were filtered off, washed with water (200 mL), and dried under reduced pressure. The solids were further purified by column chromatography (PE/EA=1/1) to give the title product (8.00 g, 53%) as a yellow solid. LCMS (Method A): 3.35 min, m/z: 618.1 [M+H]⁺. ¹H NMR (400 MHz, DMSO-d₆): 8.00 (s, 1H), 7.56 (q, J=4.8 Hz, 2H), 7.40 (dd, J=8.8, 2.0 Hz, 1H), 7.18-7.10 (m, 4H), 7.02 (t, J=52.4 Hz, 1H), 5.85 (s, 2H), 5.64 (q, J=6.4 Hz, 1H), 4.24 (s, 3H), 1.58 (d, J=6.4 Hz, 3H).

The following intermediates were similarly prepared from the appropriate Intermediate A and the appropriate Intermediate B according to the procedures for the synthesis of Intermediate A1B1.

TABLE 2
Intermediates AB

| Intermediate | Name | Structure | Analytical Data | SM |
|---|---|---|---|---|
| A1B17 | N-(4-{4-amino-7-iodo- 1-methyl-1H-pyrazolo[4,3- c]pyridin-3-yl}phenyl) ethane-1-sulfonamide | 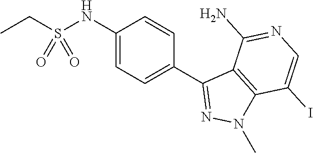 [SMILES: CCS(=O)(=O)Nc1ccc(-c2nn(C)c3c(I)cnc(N)c23)cc1] | LCMS (method A): 2.63 min, m/z 458.0 [M + H]⁺. | Intermediate A1 & Intermediate B17 |
| A1B18 | N-(4-(4-amino-7-iodo- 1-methyl-1H-pyrazolo[4,3- c]pyridin-3-yl)phenyl)-1,1- difluoromethanesulfonamide | 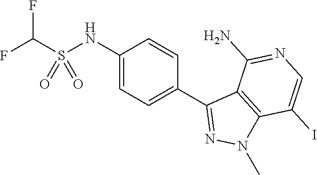 [SMILES: Cn1nc(-c2ccc(NS(=O)(=O)C(F)F)cc2)c2c(N)ncc(I)c21] | LCMS (method A): 2.80 min, m/z: 480.0 [M + H]⁺. | Intermediate A1 & Intermediate B18 |
| A1B20 | N-(4-{4-amino-7-iodo- 1-methyl-1H-pyrazolo[4,3- c]pyridin-3-yl}-2-f luorophenyl)ethane-1- sulfonamide | 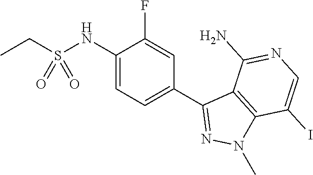 [SMILES: CCS(=O)(=O)Nc1ccc(-c2nn(C)c3c(I)cnc(N)c23)cc1F] | LCMS (method A): 2.65 min, m/z: 475.8 [M + H]⁺. | Intermediate A1 & Intermediate B20 |
| A2B19 | N-(4-(4-amino-7-iodo- 1-methyl-1H-pyrazolo[4,3- c]pyridin-3-yl)-2-((4- fluorobenzyl)oxy)phenyl) ethanesulfonamide | 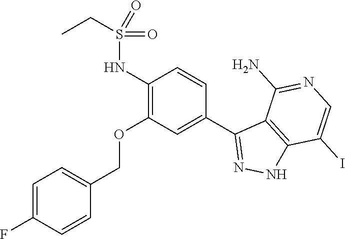 | LCMS (method A): 3.31 min, m/z: 582.0 [M + H]⁺. ¹H NMR (400 MHz, DMSO-d₆): 9.13 (s, 1H), 7.63-7.60 (m, 2H), 7.45 (d, J = 8.0 Hz, 1H), 7.33 (d, J = 1.6 Hz, 1H), 7.26-7.21 (m, 2 H, 7.18-7.15 (m, 1H), 6.08 (bs, 2H), 5.19 (s, 2H), 4.29 (s, 3H), 2.89 (q, J = 7.4 Hz, 2H), 1.12 (t, J = 7.4 Hz, 3H). | Intermediate A2 & Intermediate B19 |

### Intermediate A1B19: N-(4-(4-amino-7-iodo-1-methyl-1H-pyrazolo[4,3-c]pyridin-3-yl)-2-((4-fluorobenzyl)oxy)phenyl)ethanesulfonamide

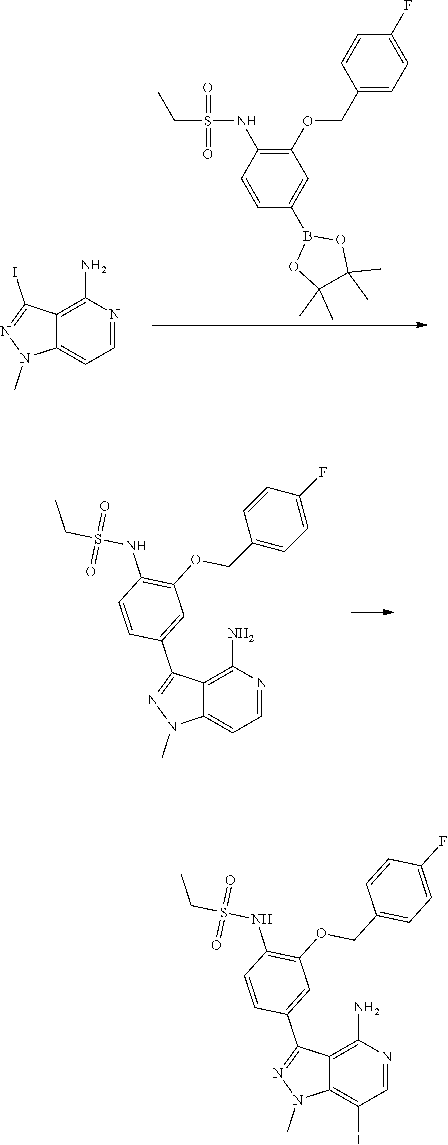 [SMILES: Cn1nc(I)c2c(N)nccc21 ; CCS(=O)(=O)Nc1ccc(B2OC(C)(C)C(C)(C)O2)cc1OCc1ccc(F)cc1 ; CCS(=O)(=O)Nc1ccc(-c2nn(C)c3ccnc(N)c23)cc1OCc1ccc(F)cc1 ; CCS(=O)(=O)Nc1ccc(-c2nn(C)c3c(I)cnc(N)c23)cc1OCc1ccc(F)cc1]

### Step 1: N-(4-{4-amino-1-methyl-1H-pyrazolo[4,3-c]pyridin-3-yl}-2-[(4-fluorophenyl)methoxy]phenyl)ethane-1-sulfonamide

A mixture of 3-iodo-1-methyl-1H-pyrazolo[4,3-c]pyridin-4-amine (50 mg, 182 μmol), N-{2-[(4-fluorophenyl)methoxy]-4-(4,4,5,5-tetramethyl-1,3,2-dioxaborolan-2-yl)phenyl}ethane-1-sulfonamide (94.8 mg, 218 μmol), Pd(dppf)Cl₂ (14.8 mg, 18.2 μmol) and Na₂CO₃ (57.8 mg, 546 μmol) in degassed 1,4-dioxane/H₂O (4/1, 10 mL) was stirred at 100° C. for 1 h under μW. The mixture was poured into water (10 mL) and extracted with EtOAc (2×10 mL). The combined organics were dried over Na₂SO₄ and concentrated under reduced pressure. The residue was purified by Prep-TLC (DCM/MeOH=20/1) to give the title product (20 mg, 24%) as a brown solid.

### Step 2: N-(4-(4-amino-7-iodo-1-methyl-1H-pyrazolo[4,3-c]pyridin-3-yl)-2-((4-fluorobenzyl) oxy)phenyl)ethanesulfonamide

A mixture of N-(4-{4-amino-1-methyl-1H-pyrazolo[4,3-c]pyridin-3-yl}-2-[(4-fluorophenyl) methoxy]phenyl)ethane-1-sulfonamide (3.5 g, 7.68 mmol) and NIS (3.44 g, 15.3 mmol) in DMF (100 mL) was stirred at 85° C. overnight. The mixture was poured into water (100 mL) and the organics were extracted with EtOAc (2×50 mL). The combined organics were dried over Na₂SO₄ and concentrated under reduced pressure. The residue was purified by column chromatography (PE/EtOAc=4/1 to 1/4, v/v) to give the title product (1.5 g, 47%) as a yellow solid.

### Intermediate A1B21: N-(4-(4-amino-7-iodo-1-methyl-1H-pyrazolo[4,3-c]pyridin-3-yl)-2-((4-fluorobenzyl)oxy)phenyl)-1,1-difluoromethanesulfonamide

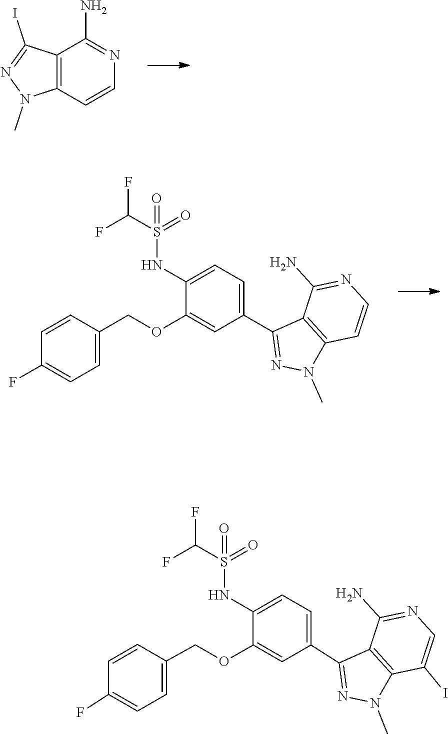 [SMILES: Cn1nc(I)c2c(N)nccc21 ; Cn1nc(-c2ccc(NS(=O)(=O)C(F)F)c(OCc3ccc(F)cc3)c2)c2c(N)nccc21 ; Cn1nc(-c2ccc(NS(=O)(=O)C(F)F)c(OCc3ccc(F)cc3)c2)c2c(N)ncc(I)c21]

### Step 1: N-(4-{4-amino-1-methyl-1H-pyrazolo[4,3-c]pyridin-3-yl}-2-[(4-fluorophenyl)methoxy]phenyl)-1,1-difluoromethanesulfonamide (Compound 10)

A mixture of methyl 3-iodo-1-methyl-1H-pyrazolo[4,3-c]pyridin-4-amine (100 mg, 0.365 mmol), 1,1-difluoro-N-{2-[(4-fluorophenyl)methoxy]-4-(4,4,5,5-tetramethyl-1,3,2-dioxaborolan-2-yl)phenyl}methanesulfonamide (166 mg, 0.365 mmol), Na₂CO₃ (77.3 mg, 0.7296 mmol) and Pd(dppf)Cl₂ (29.7 mg, 0.03648 mmol) in 1,4-dioxane/H₂O (4/1, 7.5 mL) was stirred at 100° C. for 16 h under N₂. The mixture was poured into water (100 mL) and extracted with EtOAc (2×50 mL). The combined organic phases were dried over Na₂SO₄ and concentrated. The residue was purified by column chromatography (DCM/MeOH=20/1) to give the title product (130 mg, 75%) as a grey solid. LCMS (Method A): 3.04 min, m/z: 478.1 [M+H]⁺. ¹H NMR (400 MHz, DMSO-d₆): 7.81 (d, J=8.0 Hz, 1H), 7.64-7.60 (m, 2H), 7.45-7.40 (m, 2H), 7.28-7.24 (m, 3H), 7.03-6.76 (m, 2H), 6.33 (s, 2H), 5.23 (s, 2H), 4.03 (s, 3H).

### Step 2: N-(4-(4-amino-7-iodo-1-methyl-1H-pyrazolo[4,3-c]pyridin-3-yl)-2-((4-fluorobenzyl) oxy)phenyl)-1,1-difluoromethanesulfonamide

A mixture of N-(4-{4-amino-1-methyl-1H-pyrazolo[4,3-c]pyridin-3-yl}-2-[(4-fluorophenyl) methoxy]phenyl)-1,1-difluoromethanesulfonamide (1.28 g, 2.68 mmol), NIS (661 mg, 2.94 mmol) in DMF (15 mL) was stirred at 85° C. for 16 h under N₂. The mixture was poured into water (100 mL) and extracted with EtOAc (2×50 mL). The combined organic phases were dried over Na₂SO₄ and concentrated under reduced pressure. The residue was purified by column chromatography (DCM/MeOH=20/1) to give the title product (550 mg, 34%) as a yellow solid. LCMS (Method A): 3.60 min, m/z: 604.2 [M+H]⁺.

### Intermediate A3B1: (S)—N-(4-(4-amino-7-iodo-1-isopropyl-1H-pyrazolo[4,3-c]pyridin-3-yl)-2-(1-(4-fluorophenyl)ethoxy)phenyl)-1,1-difluoromethanesulfonamide

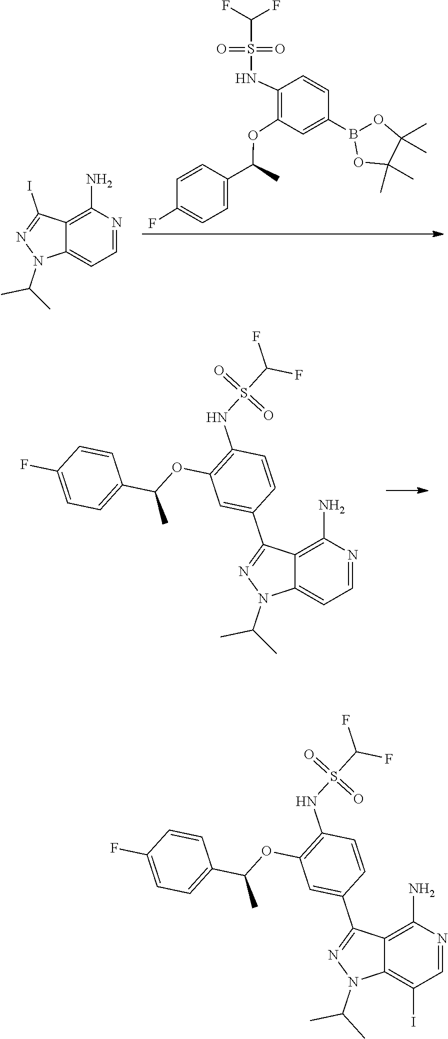

### Step 1: (S)—N-(4-(4-amino-1-isopropyl-1H-pyrazolo[4,3-c]pyridin-3-yl)-2-(1-(4-fluorophenyl)ethoxy)phenyl)-1,1-difluoromethanesulfonamide (Compound 53)

To a solution of 3-iodo-1-(propan-2-yl)-1H-pyrazolo[4,3-c]pyridin-4-amine (500 mg, 1.65 mmol) in degassed 1,4-dioxane/water (4/1, 20 mL) were added 1,1-difluoro-N-{2-[(1S)-1-(4-fluorophenyl)ethoxy]-4-(4,4,5,5-tetramethyl-1,3,2-dioxaborolan-2-yl)phenyl}methanesulfonamide (1.16 g, 1.82 mmol), Na₂CO₃ (98.9 mg, 0.9334 mmol) and Pd(dppf)Cl₂ (38.1 mg, 0.04667 mmol) and the mixture was stirred at 100° C. overnight. The mixture was cooled to RT and concentrated under reduced pressure. The residue was purified by column chromatography (DCM/MeOH=40/1) to give the title product (950 mg, >100%) as a yellow solid. LCMS (Method A): 3.17 min, m/z: 520.2 [M+H]⁺. ¹H NMR (400 MHz, DMSO-d₆): 11.32 (s, 1H), 7.76 (d, J=6.8 Hz, 1H), 7.57 (t, J=8.4 Hz, 2H), 7.40 (d, J=8.0 Hz, 1H), 7.20-7.09 (m, 5H), 7.00 (t, J=52.4 Hz, 1H), 6.55 (s, 2H), 5.64 (q, J=5.6 Hz, 1H), 4.95-4.89 (m, 1H), 1.58 (d, J=6.0 Hz, 3H), 1.45 (q, J=4.8 Hz, 6H).

### Step 2: (S)—N-(4-(4-amino-7-iodo-1-isopropyl-1H-pyrazolo[4,3-c]pyridin-3-yl)-2-(1-(4-fluorophenyl)ethoxy)phenyl)-1,1-difluoromethanesulfonamide

To a solution of N-{4-[4-amino-1-(propan-2-yl)-1H-pyrazolo[4,3-c]pyridin-3-yl]-2-[(1S)-1-(4-fluorophenyl)ethoxy]phenyl}-1,1-difluoromethanesulfonamide (980 mg, 1.88 mmol) in DMF (30 mL) was added NIS (1.05 g, 4.70 mmol) and the mixture was stirred at 85° C. overnight. Water (300 mL) was added and the solids were filtered, washed with water (200 mL) and dried under reduced pressure. The solids were further purified by column chromatography (DCM/MeOH=200/1) to give the title product (450 mg, 37%) as a brown solid. LCMS (Method A): 3.65 min. m/z: 646.1 [M+H]⁺.

### Intermediate A4B1: (S)—N-(4-(4-amino-7-bromopyrazolo[1,5-a]pyrazin-3-yl)-2-(1-(4-fluorophenyl)ethoxy)phenyl)-1,1-difluoromethanesulfonamide

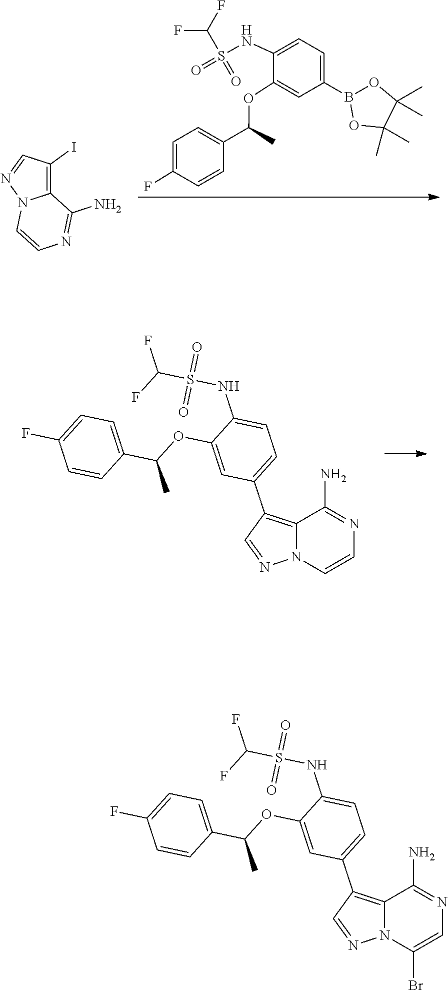

### Step 1: (S)—N-(4-(4-aminopyrazolo[1,5-a]pyrazin-3-yl)-2-(1-(4-fluorophenyl)ethoxy)phenyl)-1,1-difluoromethanesulfonamide (Compound 22)

A mixture of 3-iodopyrazolo[1,5-a]pyrazin-4-amine (2.3 g, 8.84 mmol), 1,1-difluoro-N-{2-[(1S)-1-(4-fluorophenyl)ethoxy]-4-(4,4,5,5-tetramethyl-1,3,2-dioxaborolan-2-yl)phenyl}methanesulfonamide (4.16 g, 8.84 mmol), Pd(dppf)Cl₂ (1.43 g, 1.76 mmol) and Na₂CO₃ (1.86 g, 17.6 mmol) in degassed 1,4-dioxane/H₂O (4/1, 100 mL) was stirred at 100° C. under N₂ overnight. The mixture was poured into water (100 mL) and extracted with EtOAc (2×70 mL). The combined organics were dried over Na₂SO₄ and concentrated under reduced pressure. The residue was purified by column chromatography (DCM/MeOH=80/1) to give the title product (3 g, 71%) as a brown solid. LCMS (Method A): 4.62 min, m/z: 478.1 [M+H]⁺. ¹H NMR (400 MHz, DMSO-d₆): 10.45 (s, 1H), 8.02 (d, J=4.7 Hz, 1H), 7.89 (s, 1H), 7.58 (dd, J=8.7, 5.6 Hz, 2H), 7.34 (d, J=8.0 Hz, 1H), 7.30 (d, J=4.7 Hz, 1H), 7.17 (t, J=8.9 Hz, 2H), 7.05-6.88 (m, 3H), 5.99 (s, 2H), 5.68 (q, J=6.2 Hz, 1H), 1.58 (d, J=6.3 Hz, 3H).

### Step 2: (S)—N-(4-(4-amino-7-bromopyrazolo[1,5-a]pyrazin-3-yl)-2-(1-(4-fluorophenyl)ethoxy) phenyl)-1,1-difluoromethanesulfonamide

To a solution of (S)—N-(4-(4-aminopyrazolo[1,5-a]pyrazin-3-yl)-2-(1-(4-fluorophenyl)ethoxy) phenyl)-1,1-difluoromethanesulfonamide (300 mg, 628 μmol) in DMF (10 mL) was added NBS (122 mg, 690 μmol) and the reaction was stirred at room temperature for 1 h. The mixture was poured into water (100 mL) and extracted with EtOAc (4×50 mL). The combined organics were dried over Na₂SO₄ and concentrated under reduced pressure. The residue was purified by prep-TLC (DCM/MeOH=50/1) to give the title product (100 mg, 28%) as a brown solid.

### General Procedure A1 for the Suzuki Reaction Between Intermediate A1B1 and Boronate Esters

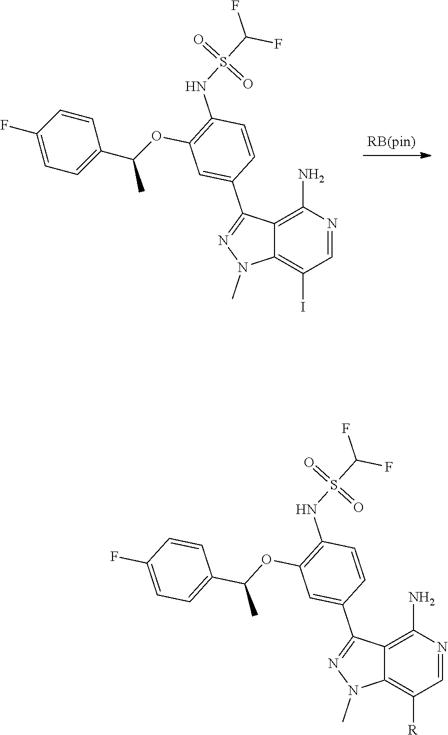

A mixture of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (1 eq), boronate ester (1.1 eq), Na₂CO₃ (3 eq) and Pd(dppf)Cl₂ (0.1 eq) in degassed 1,4-dioxane/H₂O (4/1, 0.2 M) was stirred at 100° C. under N₂ overnight (or at 80° C. for 2 h under μW irradiation). The mixture was diluted with water and extracted with EtOAc (3×). The combined organics were dried over Na₂SO₄ and concentrated under reduced pressure. The residue was purified by column chromatography (DCM/MeOH) to give the title compounds.

The following compounds were prepared following the General procedure A1 with intermediate A1B1 and corresponding boronate esters.

TABLE 3
Compounds prepared following the General procedure A with intermediate A1B1 and corresponding boronate esters

| Compound | | | | | | |
|---|---|---|---|---|---|---|
| No | Name | Structure | Yield | LCMS data | ¹H NMR data | Boronate ester |
| 24 | (S)-N-(4-(4-amino-1-methyl-7-(2- methylpyridin-4-yl)-1H- pyrazolo[4,3-c]pyridin-3-yl)-2-(1-(4- fluorophenyl)ethoxy)phenyl)-1,1- difluoromethanesulfonamide | 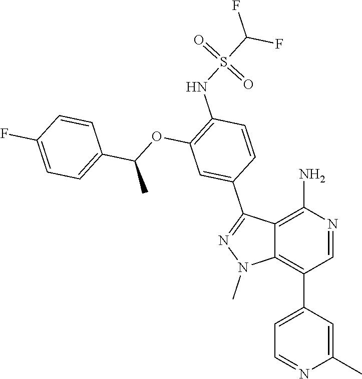 [SMILES: Cc1cc(-c2cnc(N)c3c(-c4ccc(NS(=O)(=O)C(F)F)c(O[C@@H](C)c5ccc(F)cc5)c4)nn(C)c23)ccn1] | 42% | (Method B): 2.92 min, m/z: 583.2 [M + H]⁺. | (400 MHz, DMSO-d₆): 10.59 (s, 1H), 8.50 (d, J = 5.1 Hz, 1H), 7.63 (s, 1H), 7.60-7.51 (m, 2H), 7.44- 7.36 (m, 2H), 7.30 (d, J = 5.1 Hz, 1H), 7.22-6.86 (m, 5H), 5.91 (s, 2H), 5.66 (q, J = 6.5 Hz, 1H), 3.95 (s, 1H), 3.72-3.58 (m, 3H), 2.53 (s, 3H), 1.58 (d, J = 6.3 Hz, 3H). | 2-methyl-4-(4,4,5,5- tetramethyl-1,3,2- dioxaborolan-2-yl)pyridine |
| 30 | (S)-N-(4-(4-amino-7-(1- cyclopentyl-1H-pyrazol-4-yl)-1- methyl-1H-pyrazolo[4,3-c]pyridin- 3-yl)-2-(1-(4- fluorophenyl)ethoxy)phenyl)-1,1- difluoromethanesulfonamide | 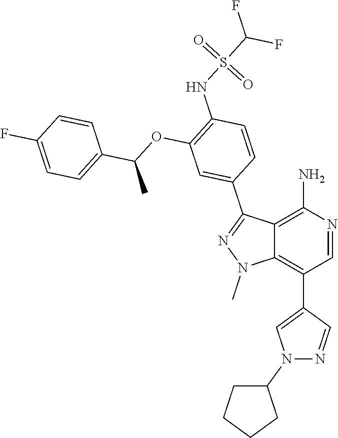 [SMILES: C[C@H](Oc1cc(-c2nn(C)c3c(-c4cnn(C5CCCC5)c4)cnc(N)c23)ccc1NS(=O)(=O)C(F)F)c1ccc(F)cc1] | 39% | (Method A): 3.14 min, m/z: 626.3 [M + H]⁺. | (400 MHz, DMSO-d₆): 7.97 (s, 1H), 7.63-7.52 (m, 4H), 7.40 (d, J = 8.0 Hz, 1H), 7.15 (dddd, J = 17.7, 9.7, 7.2, 2.1 Hz, 4H), 5.82 (s, 2H), 5.65 (d, J = 6.4 Hz, 1H), 4.75 (p, J = 6.9 Hz, 1H), 3.70 (s, 3H), 2.10 (dt, J = 12.3, 6.2 Hz, 2H), 1.97 (dqd, J = 12.9, 6.2, 3.2 Hz, 2H), 1.87-1.76 (m, 2H), 1.72- 1.62 (m, 2H), 1.58 (d, J = 6.3 Hz, 3H). | 1-cyclopentyl-4-(4,4,5,5- tetramethyl-1,3,2- dioxaborolan-2-yl)-1H- pyrazole |
| 31 | (S)-N-(4-(4-amino-7-(1-isopropyl- 1H-pyrazol-4-yl)-1-methyl-1H- pyrazolo[4,3-c]pyridin-3-yl)-2-(1-(4- fluorophenyl)ethoxy)phenyl)-1,1- difluoromethanesulfonamide | 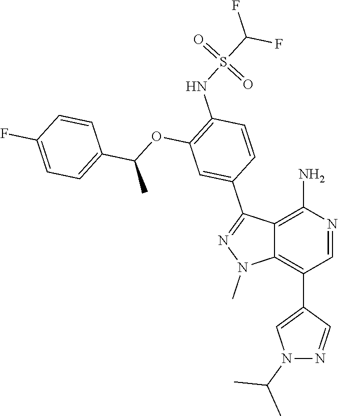 [SMILES: CC(C)n1cc(-c2cnc(N)c3c(-c4ccc(NS(=O)(=O)C(F)F)c(O[C@@H](C)c5ccc(F)cc5)c4)nn(C)c23)cn1] | 46% | (Method A): 3.22 min, m/z: 600.3 [M + H]⁺. | (400 MHz, DMSO-d₆): 10.64 (s, 1H), 7.97 (s, 1H), 7.61-7.52 (m, 4H), 7.40 (d, J = 8.0 Hz, 1H), 7.20-7.09 (m, 4H), 6.94 (d, J = 52.6 Hz, 1H), 5.79 (s, 2H), 5.65 (q, J = 6.0 Hz, 1H), 4.55 (p, J = 6.7 Hz, 1H), 3.69 (s, 3H), 1.58 (d, J = 6.2 Hz, 3H), 1.47 (d, J = 6.7 Hz, 6H). | 1-(propan-2-yl)-4-(4,4,5,5- tetramethyl-1,3,2- dioxaborolan-2-yl)-1H- pyrazole |
| 38 | (S)-N-(4-(4-amino-1-methyl-7-(1- (oxetan-3-yl)-1H-pyrazol-4-yl)-1H- pyrazolo[4,3-c]pyridin-3-yl)-2-(1-(4- fluorophenyl)ethoxy)phenyl)-1,1- difluoromethanesulfonamide | 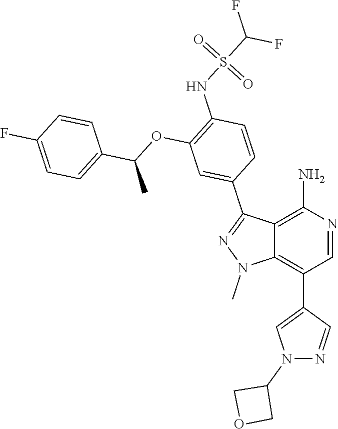 [SMILES: C[C@H](Oc1cc(-c2nn(C)c3c(-c4cnn(C5COC5)c4)cnc(N)c23)ccc1NS(=O)(=O)C(F)F)c1ccc(F)cc1] | 68% | (Method A): 2.94 min, m/z: 614.2 [M + H]⁺. | (400 MHz, DMSO-d₆): 8.12 (s, 1H), 7.77 (s, 1H), 7.56 (t, J = 8.8 Hz, 3H), 7.41 (d, J = 8.0 Hz, 1H), 7.17 (t, J = 8.8 Hz, 3H), 7.12 (d, J = 6.8 Hz, 2H), 7.02 (t, J = 52.4 Hz, 1H), 5.90 (s, 2H), 5.68-5.61 (m, 2H), 4.96 (d, J = 6.8 Hz, 4H), 3.71 (s, 3H), 1.58 (d, J = 6.4 Hz, 3H). | 1-(oxetan-3-yl)-4-(4,4,5,5- tetramethyl-1,3,2- dioxaborolan-2-yl)-1H- pyrazole |
| 19 | (S)-N-(4-(4-amino-1-methyl-7-(1- (tetrahydro-2H-pyrazin-4-yl)-1H- pyrazol-4-yl)-1H-pyrazolo[4,3- c]pyridin-3-yl)-2-(1-(4- fluorophenyl)ethoxy)phenyl)-1,1- difluoromethanesulfonamide | 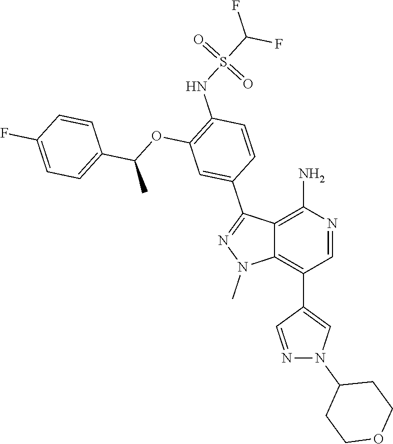 [SMILES: C[C@H](Oc1cc(-c2nn(C)c3c(-c4cnn(C5CCOCC5)c4)cnc(N)c23)ccc1NS(=O)(=O)C(F)F)c1ccc(F)cc1] | 49% | (Method A): 3.08 min, m/z: 642.2 [M + H]⁺. | (400 MHz, DMSO-d₆): 8.01 (s, 1H), 7.61 (s, 1H), 7.58-7.54 (m, 3H), 7.41 (d, J = 8.0 Hz, 1H), 7.18- 7.10 (m, 4H), 6.99 (t, J = 52.4 Hz, 1H), 5.74 (s, 2H), 5.65 (q, J = 6.4 Hz, 1H), 4.49-4.24 (m, 1H), 3.99-3.95 (m, 2H), 3.69 (s, 3H), 3.52-3.45 (m, 4H), 2.04-2.00 (m, 4H), 1.58 (d, J = 6.4 Hz, 3H) | 1-(tetrahydro-2H-pyran-4- yl)-4-(4,4,5,5-tetramethyl- 1,3,2-dioxaborolan-2-yl)- 1H-pyrazole |
| 41 | N-(4-(4-amino-1-methyl-7-(1- (tetrahydrofuran-3-yl)-1H-pyrazol- 4-yl)-1H-pyrazolo[4,3-c]pyridin-3- yl)-2-((S)-1-(4- fluorophenyl)ethoxy)phenyl)-1,1- difluoromethanesulfonamide | 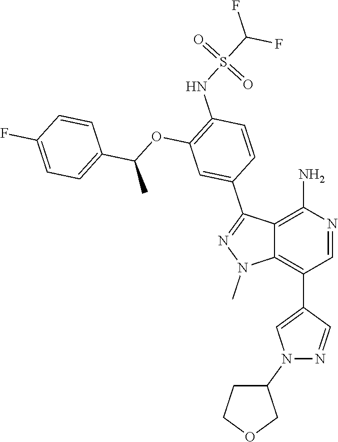 [SMILES: C[C@H](Oc1cc(-c2nn(C)c3c(-c4cnn(C5CCOC5)c4)cnc(N)c23)ccc1NS(=O)(=O)C(F)F)c1ccc(F)cc1] | 30% | (Method A): 2.97 min, m/z: 628.2 [M + H]⁺. | (400 MHz, DMSO-d₆): 10.61 (s, 1H), 8.13 (s, 1H), 7.75 (s, 1H), 7.66- 7.54 (m, 5H), 7.44 (d, J = 8.1 Hz, 1H), 7.21-6.90 (m, 6H), 5.11 (ddt, J = 9.2, 6.7, 3.5 Hz, 1H), 4.04-3.92 (m, 3H), 3.85 (td, J = 8.3, 5.3 Hz, 1H), 3.75 (s, 3H), 2.54 (s, 1H), 2.47-2.38 (m, 1H), 2.33 (dddd, J = 13.1, 8.1, 5.3, 3.6 Hz, 1H), 1.59 (d, J = 6.2 Hz, 3H). | 1-(oxolan-3-yl)-4-(4,4,5,5- tetramethyl-1,3,2- dioxaborolan-2-yl)-1H- pyrazole |
| 46 | (S)-N-(4-(4-amino-1-methyl-7-(1- (1-methylpiperidin-4-yl)-1H- pyrazol-4-yl)-1H-pyrazolo[4,3- c]pyridin-3-yl)-2-(1-(4- fluorophenyl)ethoxy)phenyl)-1,1- difluoromethanesulfonamide | 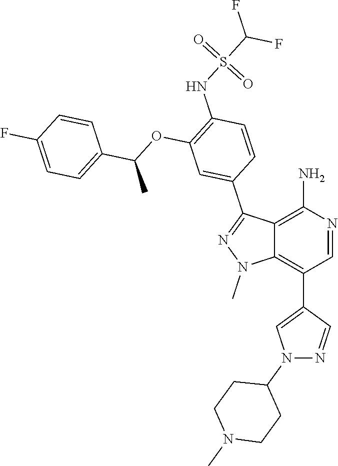 [SMILES: C[C@H](Oc1cc(-c2nn(C)c3c(-c4cnn(C5CCN(C)CC5)c4)cnc(N)c23)ccc1NS(=O)(=O)C(F)F)c1ccc(F)cc1] | 36% | (Method A): 2.53 min, m/z: 655.3 [M + H]⁺. | (400 MHz, DMSO-d₆): 7.98 (s, 1H), 7.63 (s, 1H), 7.58-7.52 (m, 3H), 7.38 (d, J = 8.0 Hz, 1H), 7.15 (t, J = 8.8 Hz, 2H), 7.05 (dd, J = 8.0, 1.6 Hz, 1H), 7.02 (d, J = 2.0 Hz, 1H), 6.77 (t, J = 53.2 Hz, 1H), 5.67 (s, 2H), 5.64 (q, J = 6 Hz, 1H), 4.40-4.32 (m, 1H), 3.68 (s, 3H), 3.23- 3.20 (m, 2H), 2.75-2.68 (m, 2H), 2.54 (s, 3H), 2.22-2.17 (m, 4H), 1.56 (d, J = 6.4 Hz, 3H) | 1-methyl-4-(4-(4,4,5,5- tetramethyl-1,3,2- dioxaborolan-2-yl)-1H- pyrazol-1-yl)piperidine |
| 21 | (S)-N-(4-(4-amino-1-methyl-7-(1- methyl-1,2,3,6-tetrahydropyridin-4- yl)-1H-pyrazolo[4,3-c]pyridin-3-yl)- 2-(1-(4- fluorophenyl)ethoxy)phenyl)-1,1- difluoromethanesulfonamide | 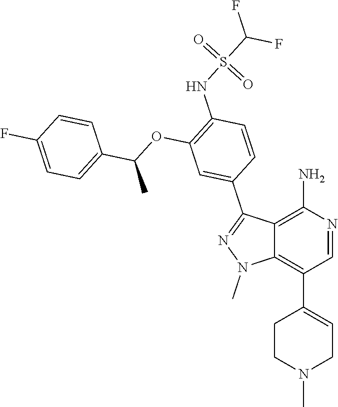 [SMILES: C[C@H](Oc1cc(-c2nn(C)c3c(C4=CCN(C)CC4)cnc(N)c23)ccc1NS(=O)(=O)C(F)F)c1ccc(F)cc1] | 58% | (Method A): 2.60 min, m/z: 587.2 [M + H]⁺. | (400 MHz, DMSO-d₆): 7.56-7.51 (m, 2H), 7.45 (s, 1H), 7.37 (d, J = 8.1 Hz, 1H), 7.15 (t, J = 8.9 Hz, 2H), 7.04 (d, J = 9.8 Hz, 1H), 7.00 (s, 1H), 6.77 (t, J = 53.3 Hz, 1H), 5.73 (s, 1H), 5.66 (s, 1H), 5.65-5.61 (m, 1H), 3.94 (s, 3H), 3.47 (s, 2H), 3.05 (s, 2H), 2.62 (s, 3H), 2.52 (d, J = 6.8 Hz, 2H), 1.55 (d, J = 6.3 Hz, 3H). | 1-methyl-4-(4,4,5,5- tetramethyl-1,3,2- dioxaborolan-2-yl)-1,2,3,6- tetrahydropyridine |
| 43 | (S)-N-(4-(4-amino-7-(3,6-dihydro- 2H-pyran-4-yl)-1-methyl-1H- pyrazolo[4,3-c]pyridin-3-yl)-2-(1-(4- fluorophenyl)ethoxy)phenyl)-1,1- difluoromethanesulfonamide | 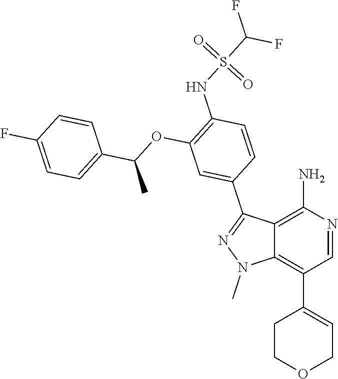 [SMILES: C[C@H](Oc1cc(-c2nn(C)c3c(C4=CCOCC4)cnc(N)c23)ccc1NS(=O)(=O)C(F)F)c1ccc(F)cc1] | 90% | (Method A): 3.14 min, m/z: 574.2 [M + H]⁺. | (400 MHz, DMSO-d₆): 7.57 (dd, J = 8.7, 5.6 Hz, 2H), 7.48 (s, 1H), 7.40 (d, J = 8.0 Hz, 1H), 7.17 (t, J = 8.9 Hz, 2H), 7.14-7.09 (m, 2H), 6.95 (d, J = 52.6 Hz, 1H), 5.87 (s, 1H), 5.84 (s, 1H), 5.65 (q, J = 6.2 Hz, 1H), 4.23 (d, J = 2.5 Hz, 2H), 3.97 (s, 3H), 3.86 (t, J = 5.3 Hz, 2H), 2.34 (s, 2H), 1.57 (d, J = 6.3 Hz, 3H). | 2-(3,6-dihydro-2H-pyran-4- yl)-4,4,5,5-tetramethyl- 1,3,2-dioxaborolane |
| 28 | (S)-N-(4-(4-amino-7-(6- methoxypyridin-3-yl)-1-methyl-1H- pyrazolo[4,3-c]pyridin-3-yl)-2-(1-(4- fluorophenyl)ethoxy)phenyl)-1,1- difluoromethanesulfonamide | 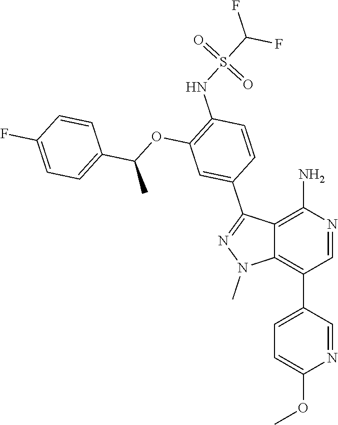 [SMILES: COc1ccc(-c2cnc(N)c3c(-c4ccc(NS(=O)(=O)C(F)F)c(O[C@@H](C)c5ccc(F)cc5)c4)nn(C)c23)cn1] | 69% | (Method A): 3.24 min, m/z: 599.2 [M + H]⁺. | (400 MHz, DMSO-d₆): 10.98 (s, 1H), 8.94 (d, J = 2.2 Hz, 1H), 8.80 (dd, J = 4.8, 1.6 Hz, 1H), 8.21- 8.14 (m, 2H), 7.69-7.57 (m, 2H), 7.55 (s, 1H), 7.41 (s, 1H), 7.21 (dd, J = 8.3, 1.3 Hz, 1H), 6.17 (s, 2H), 4.08 (d, J = 7.2 Hz, 2H), 3.88 (s, 3H), 3.85- 3.79 (m, 2H), 3.23 (td, J = 11.6, 2.1 Hz, 2H), 2.13 (ddp, J = 11.4, 7.9, 3.7 Hz, 1H), 1.46-1.38 (m, 2H), 1.29 (td, J = 12.3, 4.4 Hz, 2H). | 2-methoxy-5-(4,4,5,5- tetramethyl-1,3,2- dioxaborolan-2-yl)pyridine |
| 26 | (S)-N-(4-(4-amino-1-methyl-7-(6- methylpyridin-3-yl)-1H- pyrazolo[4,3-c]pyridin-3-yl)-2-(1-(4- fluorophenyl)ethoxy)phenyl)-1,1- difluoromethanesulfonamide | 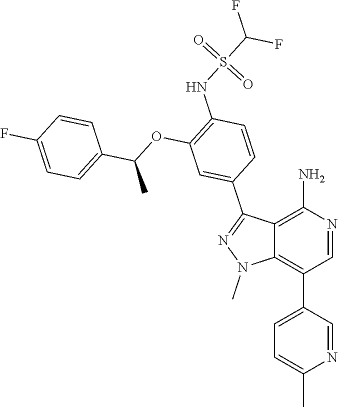 [SMILES: Cc1ccc(-c2cnc(N)c3c(-c4ccc(NS(=O)(=O)C(F)F)c(O[C@@H](C)c5ccc(F)cc5)c4)nn(C)c23)cn1] | 50% | (Method A): 2.80 min, m/z: 583.2 [M + H]⁺. | (400 MHz, DMSO-d₆): 8.53 (d, J = 2.3 Hz, 1H), 7.80 (dd, J = 7.9, 2.4 Hz, 1H), 7.57 (q, J = 5.6, 6.0 Hz, 3H), 7.42 (d, J = 8.0 Hz, 1H), 7.36 (d, J = 7.9 Hz, 1H), 7.21-7.12 (m, 4H), 5.95 (s, 2H), 5.66 (d, J = 6.3 Hz, 1H), 3.56 (s, 3H), 2.54 (s, 3H), 1.58 (d, J = 6.2 Hz, 3H). | 2-methyl-5-(4,4,5,5- tetramethyl-1,3,2- dioxaborolan-2-yl)pyridine |
| 25 | (S)-N-(4-(4-amino-1-methyl-7- (pyridin-3-yl)-1H-pyrazolo[4,3- c]pyridin-3-yl)-2-(1-(4- fluorophenyl)ethoxy)phenyl)-1,1- difluoromethanesulfonamide | 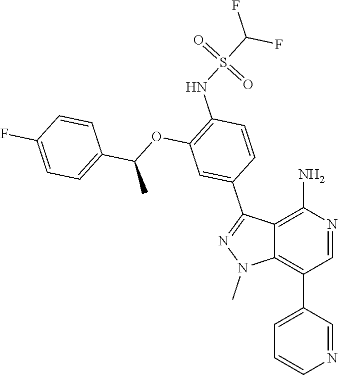 [SMILES: C[C@H](Oc1cc(-c2nn(C)c3c(-c4cccnc4)cnc(N)c23)ccc1NS(=O)(=O)C(F)F)c1ccc(F)cc1] | 49% | (Method A): 2.94 min, m/z: 569.2 [M + H]⁺. | (400 MHz, DMSO-d₆): 8.68 (d, J = 2.3 Hz, 1H), 8.63 (dd, J = 4.8, 1.7 Hz, 1H), 7.93 (dt, J = 7.8, 2.0 Hz, 1H), 7.61 (s, 1H), 7.59-7.54 (m, 2H), 7.51 (dd, J = 7.8, 4.8 Hz, 1H), 7.42 (d, J = 8.0 Hz, 1H), 7.20-6.89 (m, 5H), 5.99 (s, 2H), 5.65 (q, J = 6.2 Hz, 1H), 3.56 (s, 3H), 1.58 (d, J = 6.3 Hz, 3H). | 3-(4,4,5,5-tetramethyl- 1,3,2-dioxaborolan-2- yl)pyridine |
| 32 | (S)-N-(4-(4-amino-1-methyl-7-(3- methylpyridin-4-yl)-1H- pyrazolo[4,3-c]pyridin-3-yl)-2-(1-(4- fluorophenyl)ethoxy)phenyl)-1,1- difluoromethanesulfonamide | 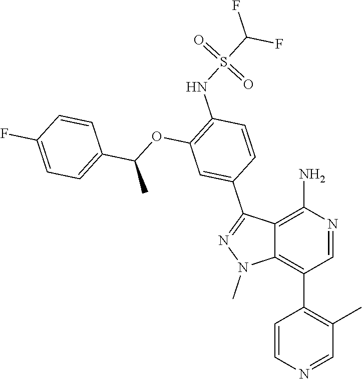 [SMILES: Cc1cnccc1-c1cnc(N)c2c(-c3ccc(NS(=O)(=O)C(F)F)c(O[C@@H](C)c4ccc(F)cc4)c3)nn(C)c12] | 25% | (Method A): 3.04 min, m/z: 583.2 [M + H]⁺. | (400 MHz, MeOD-d₄): 8.54 (s, 1H), 8.48 (d, J = 5.0 Hz, 1H), 7.49 (dd, J = 5.8, 2.3 Hz, 3H), 7.39 (dd, J = 5.1, 2.0 Hz, 1H), 7.17 (d, J = 7.9 Hz, 1H), 7.11-7.03 (m, 3H), 6.64 (s, 1H), 5.57 (d, J = 6.4 Hz, 1H), 3.48 (s, 3H), 2.17 (s, 3H), 1.69 (d, J = 6.4 Hz, 3H). | 3-methylpyridin-4-ylboronic acid |
| 27 | (S)-N-(4-(4-amino-7-(2- methoxypyridin-4-yl)-1-methyl-1H- pyrazolo[4,3-c]pyridin-3-yl)-2-(1-(4- fluorophenyl)ethoxy)phenyl)-1,1- difluoromethanesulfonamide | 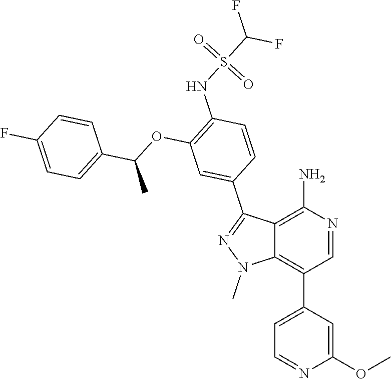 [SMILES: COc1cc(-c2cnc(N)c3c(-c4ccc(NS(=O)(=O)C(F)F)c(O[C@@H](C)c5ccc(F)cc5)c4)nn(C)c23)ccn1] | 63% | (Method A): 3.25 min, m/z: 599.2 [M + H]⁺. | (400 MHz, DMSO-d₆): 8.22 (d, J = 4.4 Hz, 1H), 9.59 (s, 1H), 7.55 (m, 3H), 7.18 (d, J = 7.6 Hz, 1H), 7.17 (d, J = 7.6 Hz, 2H), 7.11 (m, 3H), 6.89 (s, 1H), 6.83 (s, J = 52.3 Hz, 1H), 5.57 (d, J = 6.0 Hz, 1H), 5.49 (s, 1H), 3.97 (s, 3H), 3.68 (s, 3H), 1.69 (d, J = 5.6 Hz, 1H) | 2-methoxy-4-(4,4,5,5- tetramethyl-1,3,2- dioxaborolan-2-yl)pyridine |
| 23 | (S)-N-(4-(4-amino-1-methyl-7-(2- (trifluoromethyl)pyridin-4-yl)-1H- pyrazolo[4,3-c]pyridin-3-yl)-2-(1-(4- fluorophenyl)ethoxy)phenyl)-1,1- difluoromethanesulfonamide | 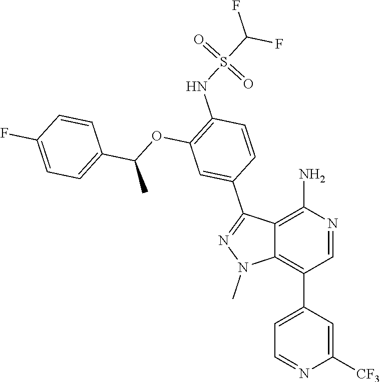 [SMILES: C[C@H](Oc1cc(-c2nn(C)c3c(-c4ccnc(C(F)(F)F)c4)cnc(N)c23)ccc1NS(=O)(=O)C(F)F)c1ccc(F)cc1] | 50% | (Method A): 3.26 min, m/z: 637.2 [M + H]⁺. | (400 MHz, DMSO-d₆): 10.58 (s, 1H), 8.81 (d, J = 5.0 Hz, 1H), 8.06 (s, 1H), 7.85 (d, J = 5.9 Hz, 1H), 7.75 (s, 1H), 7.57 (dd, J = 8.6, 5.6 Hz, 2H), 7.42 (d, J = 8.0 Hz, 1H), 7.21- 7.13 (m, 3H), 7.12-6.89 (m, 2H), 6.07 (s, 2H), 5.65 (d, J = 6.3 Hz, 1H), 3.62 (s, 3H), 1.59 (d, J = 6.3 Hz, 3H). | 4-(4,4,5,5-tetramethyl- 1,3,2-dioxaborolan-2-yl)-2- (trifluoromethyl)pyridine |
| 57 | (S)-N-(4-(4-amino-7-(2- (difluoromethyl)pyridin-4-yl)-1- methyl-1H-pyrazolo[4,3-c]pyridin- 3-yl)-2-(1-(4- fluorophenyl)ethoxy)phenyl)-1,1- difluoromethanesulfonamide | 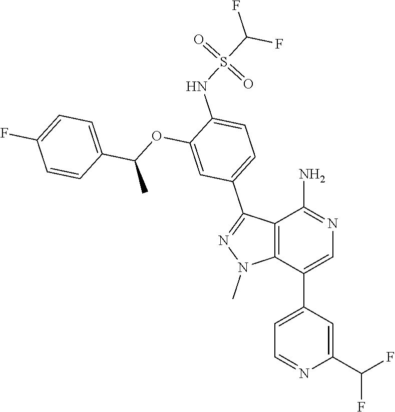 [SMILES: C[C@H](Oc1cc(-c2nn(C)c3c(-c4ccnc(C(F)F)c4)cnc(N)c23)ccc1NS(=O)(=O)C(F)F)c1ccc(F)cc1] | 34% | (Method A): 3.28 min, m/z: 619.2 [M + H]⁺. | (400 MHz, DMSO-d₆): 8.74 (d, J = 5.2 Hz, 1H), 7.80 (s, 1H), 7.71 (s, 2H), 7.57 (q, J = 4.8 Hz, 2H), 7.42 (d, J = 8.0 Hz, 1H), 7.19-7.12 (m, 4H), 7.01 (t, J = 52.8 Hz, 1H), 5.99 (s, 2H), 5.66 (q, J = 6.0 Hz, 1H), 3.62 (s, 3H), 1.59 (d, J = 6.4 Hz, 3H). | 2-(difluoromethyl)-4- (4,4,5,5-tetramethyl-1,3,2- dioxaborolan-2-yl)pyridine |
| 48 | (S)-N-(4-(4-amino-7-(2-(tert- butyl)pyridin-4-yl)-1-methyl-1H- pyrazolo[4,3-c]pyridin-3-yl)-2-(1-(4- fluorophenyl)ethoxy)phenyl)-1,1- difluoromethanesulfonamide | 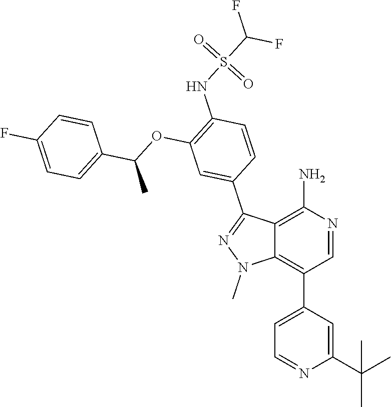 [SMILES: C[C@H](Oc1cc(-c2nn(C)c3c(-c4ccnc(C(C)(C)C)c4)cnc(N)c23)ccc1NS(=O)(=O)C(F)F)c1ccc(F)cc1] | 22% | (Method A): 3.42 min, m/z: 625.6 [M + H]⁺. | (400 MHz, MeOH-d₄): 8.58 (dd, J = 5.0, 0.8 Hz, 1H), 7.61 (s, 1H), 7.57- 7.53 (m, 2H), 7.49 (dd, J = 8.6, 5.4 Hz, 2H), 7.33 (dd, J = 5.1, 1.7 Hz, 1H), 7.19 (dd, J = 8.1, 1.8 Hz, 1H), 7.12 (d, J = 1.8 Hz, 1H), 7.06 (t, J = 8.8 Hz, 2H), 6.70 (s, 1H), 5.57 (q, J = 6.3 Hz, 1H), 3.65 (s, 3H), 1.70 (d, J = 6.4 Hz, 3H), 1.42 (s, 9H). | 2-(tert-butyl)-4-(4,4,5,5- tetramethyl-1,3,2- dioxaborolan-2-yl)pyridine |
| 49 | (S)-N-(4-(4-amino-7-(2- (dimethylamino)pyridin-4-yl)-1- methyl-1H-pyrazolo[4,3-c]pyridin- 3-yl)-2-(1-(4- fluorophenyl)ethoxy)phenyl)-1,1- difluoromethanesulfonamide | 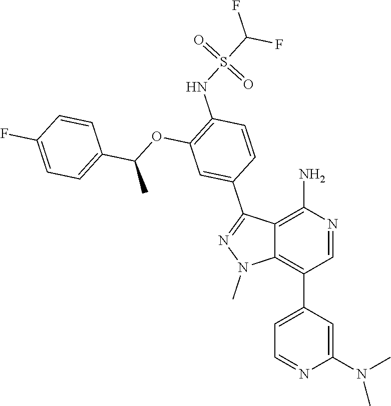 [SMILES: C[C@H](Oc1cc(-c2nn(C)c3c(-c4ccnc(N(C)C)c4)cnc(N)c23)ccc1NS(=O)(=O)C(F)F)c1ccc(F)cc1] | 57% | (Method A): 2.75 min, m/z: 612.2 [M + H]⁺. | (400 MHz, DMSO-d₆): 8.14 (d, J = 4.8 Hz, 1H), 7.64 (s, 1H), 7.57 (t, J = 5.6 Hz, 2H), 7.42 (d, J = 8.0 Hz, 1H), 7.19-7.12 (m, 4H), 7.03 (t, J = 52.4 Hz, 1H), 6.67 (s, 2H), 6.02 (s, 2H), 5.65 (q, J = 6.0 Hz, 1H), 3.66 (s, 3H), 3.07 (s, 6H), 1.59 (d, J = 6.0 Hz, 3H). | N,N-dimethyl-4-(4,4,5,5- tetramethyl-1,3,2- dioxaborolan-2-yl)pyridin- 2-amine |
| 29 | (S)-N-(4-(4-amino-7-(1-(2- methoxyethyl)-1H-pyrazol-4-yl)-1- methyl-1H-pyrazolo[4,3-c]pyridin- 3-yl)-2-(1-(4- fluorophenyl)ethoxy)phenyl)-1,1- difluoromethanesulfonamide | 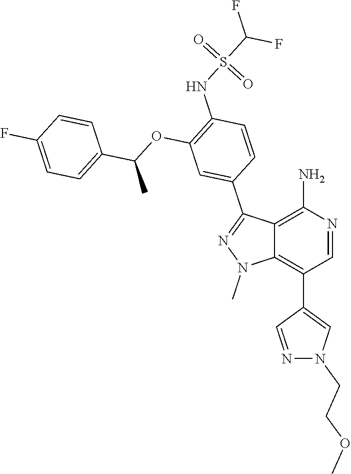 [SMILES: COCCn1cc(-c2cnc(N)c3c(-c4ccc(NS(=O)(=O)C(F)F)c(O[C@@H](C)c5ccc(F)cc5)c4)nn(C)c23)cn1] | 60% | (Method A): 2.99 min, m/z: 616.2 [M + H]⁺. | (400 MHz, DMSO-d₆): 7.93 (s, 1H), 7.61 (s, 1H), 7.59-7.55 (m, 3H), 7.41 (d, J = 8.0 Hz, 1H), 7.19- 7.12 (m, 4H), 7.02 (t, J = 52.4 Hz, 1H), 5.94 (s, 2H), 5.65 (q, J = 6.0 Hz, 1H), 4.32 (t, J = 5.6 Hz, 2H), 3.73 (t, J = 5.6 Hz, 2H), 3.70 (s, 3H), 1.58 (q, J = 6.0 Hz, 3H). | 1-(2-methoxyethyl)-4- (4,4,5,5-tetramethyl-1,3,2- dioxaborolan-2-yl)-1H- pyrazole |
| 33 | (S)-N-(4-(4-amino-7-(3,5- dimethylisoxazol-4-yl)-1-methyl- 1H-pyrazolo[4,3-c]pyridin-3-yl)-2- (1-(4-fluorophenyl)ethoxy)phenyl)- 1,1-difluoromethanesulfonamide | 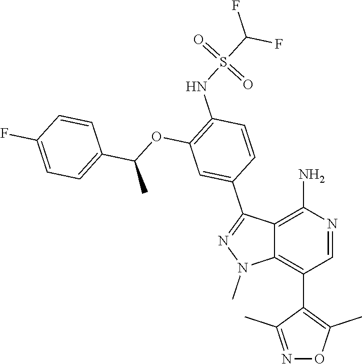 [SMILES: Cc1noc(C)c1-c1cnc(N)c2c(-c3ccc(NS(=O)(=O)C(F)F)c(O[C@@H](C)c4ccc(F)cc4)c3)nn(C)c12] | 21% | (Method A): 3.15 min, m/z: 587.2 [M + H]⁺. | (400 MHz, MeOD-d₄): δ 10.82 (s, 1H), 7.58 (t, J = 8.4 Hz, 2H), 7.55 (s, 1H), 7.40 (d, J = 8.4 Hz, 1H), 7.18 (t, J = 8.8 Hz, 4H), 7.14-6.88 (m, 1H), 6.06 (s, 2H), 5.68 (q, J = 6.0 Hz, 1H), 3.61 (s, 3H), 2.28 (s, 3H), 2.06 (s, 3H), 1.57 (d, J = 6.0 Hz, 3H). | 3,5-dimethyl-4-(4,4,5,5- tetramethyl-1,3,2- dioxaborolan-2-yl)-1,2- oxazole |
| 15 | (S)-N-(4-(4-amino-7-(1-(2- hydroxyethyl)-1H-pyrazol-4-yl)-1- methyl-1H-pyrazolo[4,3-c]pyridin- 3-yl)-2-(1-(4- fluorophenyl)ethoxy)phenyl)-1,1- difluoromethanesulfonamide | 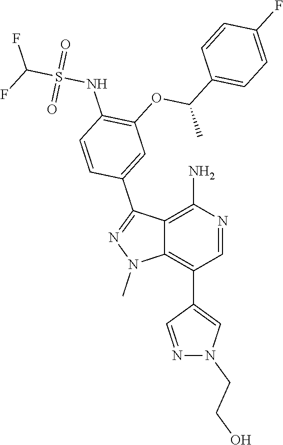 [SMILES: C[C@H](Oc1cc(-c2nn(C)c3c(-c4cnn(CCO)c4)cnc(N)c23)ccc1NS(=O)(=O)C(F)F)c1ccc(F)cc1] | 51% | (Method A): 3.23 min, m/z: 602.2 [M + H]⁺. | (400 MHz, DMSO-d₆): 7.90 (s, 1H), 7.60-7.54 (m, 4H), 7.41 (d, J = 8.0 Hz, 1H), 7.19-6.88 (m, 5H), 5.81 (s, 2H), 5.68- 5.63 (m, 1H), 4.94 (t, J = 4.4 Hz, 1H), 4.20 (t, J = 5.6 Hz, 2H), 3.80-3.77 (m, 2H), 3.71 (s, 3H), 1.58 (d, J = 6.4 Hz, 3H). | 2-[4-(4,4,5,5-tetramethyl- 1,3,2-dioxaborolan-2-yl)- 1H-pyrazol-1-yl)ethan-1-ol |
| 20 | (S)-N-(4-(4-amino-1-methyl-7-(1- methyl-1H-pyrazol-4-yl)-1H- pyrazolo[4,3-c]pyridin-3-yl)-2-(1-(4- fluorophenyl)ethoxy)phenyl)-1,1- difluoromethanesulfonamide | 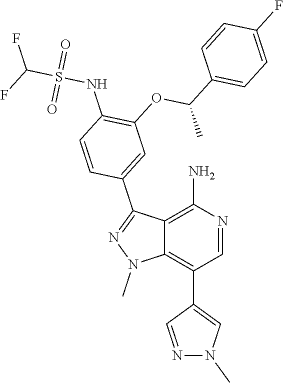 [SMILES: C[C@H](Oc1cc(-c2nn(C)c3c(-c4cnn(C)c4)cnc(N)c23)ccc1NS(=O)(=O)C(F)F)c1ccc(F)cc1] | 35% | (Method A): 3.04 min, m/z: 572.2 [M + H]⁺. | (400 MHz, DMSO-d₆): 7.88 (s, 1H), 7.61-7.52 (m, 4H), 7.40 (d, J = 8.0 Hz, 1H), 7.20-6.84 (m, 5H), 5.89 (s, 1H), 5.63 (q, J = 6.2 Hz, 1H), 3.90 (s, 3H), 3.71 (s, 3H), 1.57 (d, J = 6.2 Hz, 3H). | 1-methyl-4-(4,4,5,5- tetramethyl-1,3,2- dioxaborolan-2-yl)-1H- pyrazole |
| 18 | (S)-N-(4-(4-amino-1-methyl-7- (pyridin-4-yl)-1H-pyrazolo[4,3- c]pyridin-3-yl)-2-(1-(4- fluorophenyl)ethoxy)phenyl)-1,1- difluoromethanesulfonamide | 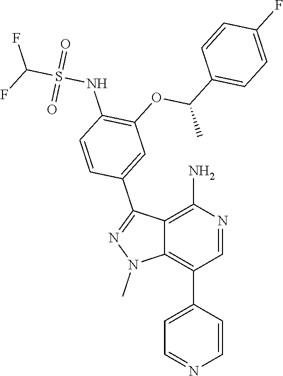 [SMILES: C[C@H](Oc1cc(-c2nn(C)c3c(-c4ccncc4)cnc(N)c23)ccc1NS(=O)(=O)C(F)F)c1ccc(F)cc1] | 42% | (Method A): 2.95 min, m/z: 569.2 [M + H]⁺. | (400 MHz, DMSO-d₆): 8.64 (d, J = 6.0 Hz, 2H), 7.64 (s, 1H), 7.56 (dd, J = 8.6, 5.6 Hz, 2H), 7.53- 7.50 (m, 2H), 7.41 (d, J = 8.0 Hz, 1H), 7.20-6.86 (m, 5H), 5.94 (s, 2H), 5.65 (q, J = 6.3 Hz, 1H), 3.61 (s, 3H), 1.58 (d, J = 6.3 Hz, 3H). | 4-(4,4,5,5-tetramethyl- 1,3,2-dioxaborolan-2- yl)pyridine |
| 34 | (S)-N-(4-(4-amino-7-benzyl-1- methyl-1H-pyrazolo[4,3-c]pyridin- 3-yl)-2-(1-(4- fluorophenyl)ethoxy)phenyl)-1,1- difluoromethanesulfonamide | 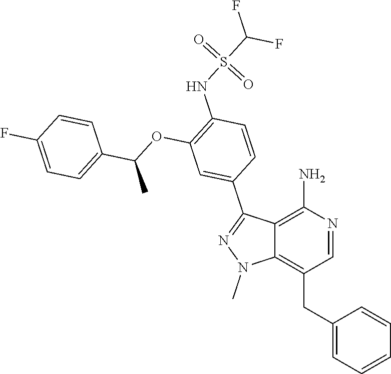 [SMILES: C[C@H](Oc1cc(-c2nn(C)c3c(Cc4ccccc4)cnc(N)c23)ccc1NS(=O)(=O)C(F)F)c1ccc(F)cc1] | 21% | (Method A): 3.35 min, m/z: 582.2 [M + H]⁺. | (400 MHz, DMSO-d₆ ): 11.35 (s, 1H), 7.56 (s, 3H), 7.39 (d, J = 8.4 Hz, 1H), 7.33 (d, J = 7.2 Hz, 2H), 7.25-7.10 (m, 8H), 6.98 (t, J = 52.4 Hz, 1H), 6.27 (s, 2H), 5.65 (q, J = 6.0 Hz, 1H), 4.31 (s, 2H), 3.89 (s, 3H), 1.56 (d, J = 6.4 Hz, 3H). | 2-benzyl-4,4,5,5- tetramethyl-1,3,2- dioxaborolane |
| 61 | (S)-N-(4-(4-amino-1-methyl-7-(1- (1-(2,2,2-trifluoroethyl)piperidin-4- yl)-1H-pyrazol-4-yl)-1H- pyrazolo[4,3-c]pyridin-3-yl)-2-(1-(4- fluorophenyl)ethoxy)phenyl)-1,1- difluoromethanesulfonamide | 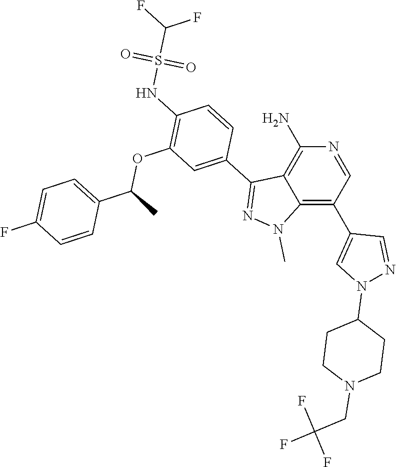 [SMILES: C[C@H](Oc1cc(-c2nn(C)c3c(-c4cnn(C5CCN(CC(F)(F)F)CC5)c4)cnc(N)c23)ccc1NS(=O)(=O)C(F)F)c1ccc(F)cc1] | 51% | (Method A): 2.95 min, m/z: 723.2 [M + H]⁺. | (400 MHz, DMSO-d₆): 10.62 (bs, 1H), 8.00 (s, 1H), 7.60-7.55 (m, 4H), 7.41-7.39 (m, 1H), 7.20- 6.86 (m, 5H), 5.73 (bs, 2H), 5.65 (q, J = 12.8, 6.4 Hz, 1H), 4.26-4.19 (m, 1H), 3.30-3.20 (m, 5H), 3.06-3.02 (m, 2H), 2.59- 2.52 (m, 2H), 2.05-1.96 (m, 4H), 1.58 (d, J = 6.4 Hz, 3H). | Intermediate C1 |
| 60 | (S)-N-(4-(4-amino-1-methyl-7-(1- (2-(4-metyhlpiperazin-1-yl)ethyl)- 1H-pyrazol-4-yl)-1H-pyrazolo[4,3- c]pyridin-3-yl)-2-(1-(4- fluorophenyl)ethoxy)phenyl)-1,1- difluoromethanesulfonamide | 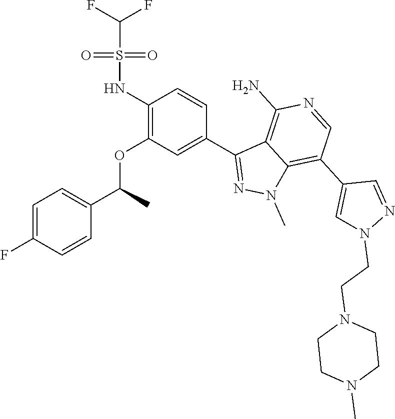 [SMILES: C[C@H](Oc1cc(-c2nn(C)c3c(-c4cnn(CCN5CCN(C)CC5)c4)cnc(N)c23)ccc1NS(=O)(=O)C(F)F)c1ccc(F)cc1] | 17% | (Method A): 2.55 min, m/z: 684.1 [M + H]⁺. | (400 MHz, DMSO-d₆): 7.92 (s, 1H), 7.57-7.52 (m, 4H), 7.39 (d, J = 8.0 Hz, 1H), 7.16 (t, J = 8.8 Hz, 2H), 7.07 (dd, J = 8.0, 1.6 Hz, 1H), 7.03 (s, 1H), 6.82 (t, J = 53.2 Hz, 1H), 5.66-5.63 (m, 3H), 4.28 (t, J = 6.0 Hz, 2H), 3.70 (s, 3H), 2.79 (t, J = 6.4 Hz, 2H), 2.70 (bs, 4H), 2.55 (bs, 4H), 2.44 (s, 3H), 1.56 (d, J = 6.0 Hz, 3H). | Intermediate C2 |
| 84 | (S)-N-(4-(4-amino-7-(3- chlorophenyl)-1-methyl-1H- pyrazolo[4,3-c]pyridin-3-yl)-2-(1-(4- fluorophenyl)ethoxy)phenyl)-1,1- difluoromethanesulfonamide | 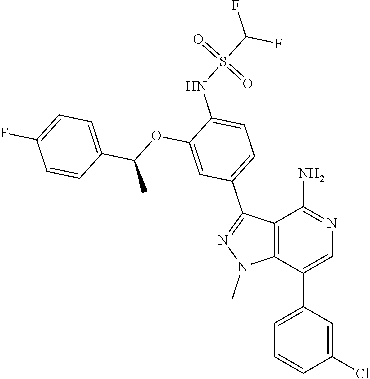 [SMILES: C[C@H](Oc1cc(-c2nn(C)c3c(-c4cccc(Cl)c4)cnc(N)c23)ccc1NS(=O)(=O)C(F)F)c1ccc(F)cc1] | 26% | (Method A): 2.99 min, m/z: 602.1 [M + H]⁺. | (400 MHz, DMSO-d₆) 10.60 (s, 1H), 7.53 (dt, J = 25.8, 8.3 Hz, 7H), 7.41 (d, J = 7.9 Hz, 1H), 7.21- 7.08 (m, 4H), 5.83 (s, 2H), 5.66 (q, J = 6.3 Hz, 1H), 3.55 (s, 3H), 1.58 (d, J = 6.1 Hz, 3H). | 2-(3-chlorophenyl)-4,4,5,5- tetramethyl-1,3,2- dioxaborolane |
| 85 | (S)-N-(4-(4-amino-7-(2- chlorophenyl)-1-methyl-1H- pyrazolo[4,3-c]pyridin-3-yl)-2-(1-(4- fluorophenyl)ethoxy)phenyl)-1,1- difluoromethanesulfonamide | 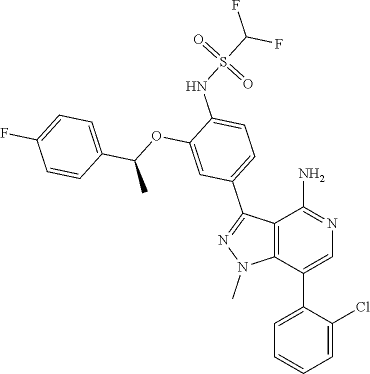 [SMILES: C[C@H](Oc1cc(-c2nn(C)c3c(-c4ccccc4Cl)cnc(N)c23)ccc1NS(=O)(=O)C(F)F)c1ccc(F)cc1] | 35% | (Method A): 2.93 min, m/z: 602.1 [M + H]⁺. | (400 MHz, DMSO-d₆) 7.63 (dd, J = 6.3, 3.0 Hz, 1H), 7.59 (td, J = 5.7, 2.7 Hz, 2H), 7.55 (s, 1H), 7.50 (qd, J = 6.4, 5.6, 2.1 Hz, 2H), 7.45-7.39 (m, 1H), 7.22-7.17 (m, 3H), 7.16-6.88 (m, 1H), 6.01 (s, 2H), 5.76-5.66 (m, 1H), 3.46 (s, 3H), 1.58 (d, J = 6.2 Hz, 3H). | 2-(2-chlorophenyl)-4,4,5,5- tetramethyl-1,3,2- dioxaborolane |
| 86 | (S)-N-(4-(4-amino-1-methyl-7- phenyl-1H-pyrazolo[4,3-c]pyridin- 3-yl)-2-(1-(4- fluorophenyl)ethoxy)phenyl)-1,1- difluoromethanesulfonamide | 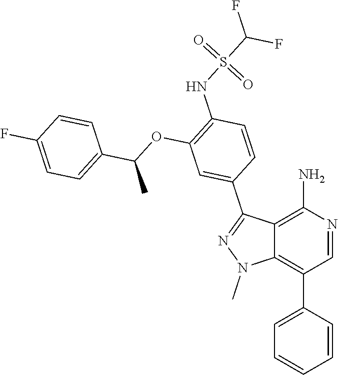 [SMILES: C[C@H](Oc1cc(-c2nn(C)c3c(-c4ccccc4)cnc(N)c23)ccc1NS(=O)(=O)C(F)F)c1ccc(F)cc1] | 55% | (Method A): 2.81 min, m/z: 568.2 [M + H]⁺. | (400 MHz, DMSO-d₆) 10.80 (s, 1H), 7.60-7.56 (m, 3H), 7.58-7.41 (m, 6H), 7.20-7.15 (m, 4H), 7.03 (t, J = 52 Hz, 1H), 6.04 (s, 2H), 5.66 (d, J = 8 Hz, 1H), 3.53 (s, 3H), 1.59 (t, J = 8 Hz, 3H). | phenylboronic acid |
| 87 | (S)-N-(4-(4-amino-7-(4- chlorophenyl)-1-methyl-1H- pyrazolo[4,3-c]pyridin-3-yl)-2-(1-(4- fluorophenyl)ethoxy)phenyl)-1,1- difluoromethanesulfonamide | 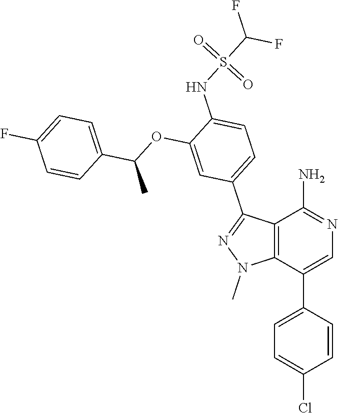 [SMILES: C[C@H](Oc1cc(-c2nn(C)c3c(-c4ccc(Cl)cc4)cnc(N)c23)ccc1NS(=O)(=O)C(F)F)c1ccc(F)cc1] | 27% | (Method A): 2.99 min, m/z: 602.1 [M + H]⁺. | (400 MHz, DMSO-d₆) 10.60 (s, 1H), 7.53 (dt, J = 25.8, 8.3 Hz, 7H), 7.41 (d, J = 7.9 Hz, 1H), 7.21- 7.08 (m, 4H), 5.83 (s, 2H), 5.66 (q, J = 6.3 Hz, 1H), 3.55 (s, 3H), 1.58 (d, J = 6.1 Hz, 3H). | 2-(4-chlorophenyl)-4,4,5,5- tetramethyl-1,3,2- dioxaborolane |
| 105 | (S)-N-(4-(4-amino-1-methyl-7-(2- (4-methylpiperazin-1-yl)pyrimidin- 5-yl)-1H-pyrazolo[4,3-c]pyridin-3- yl)-2-(1-(4- fluorophenyl)ethoxy)phenyl)-1,1- difluoromethanesulfonamide | 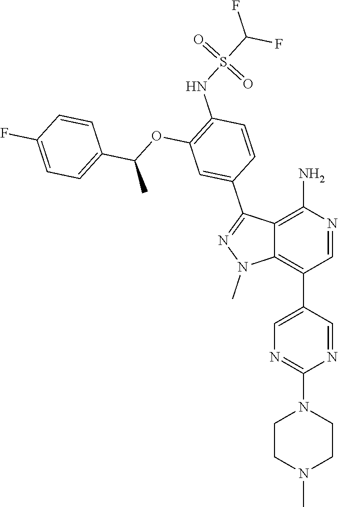 [SMILES: C[C@H](Oc1cc(-c2nn(C)c3c(-c4cnc(N5CCN(C)CC5)nc4)cnc(N)c23)ccc1NS(=O)(=O)C(F)F)c1ccc(F)cc1] | 46% | (Method A): 2.65 min, m/z: 669.0 [M + H]⁺. | (400 MHz, DMSO-d₆) 8.29 (s, 1H), 8.16 (s, 1H), 7.82 (d, J = 8.3 Hz, 1H), 7.58 (s, 1H), 7.42 (s, 1H), 7.24 (d, J = 8.3 Hz, 1H), 6.19 (s, 2H), 4.08 (d, J = 7.2 Hz, 2H), 3.96 (s, 3H), 3.53 (q, J = 6.8 Hz, 4H), 2.81 (d, J = 11.1 Hz, 2H), 2.19 (s, 3H), 1.95-1.88 (m, 2H), 1.50 (d, J = 12.4 Hz, 2H), 1.33 (d, J = 6.8 Hz, 6H). | 2-(4-methylpiperazin-1-yl)- 5-(4,4,5,5-tetramethyl- 1,3,2-dioxaborolan-2- yl)pyrimidine |
| 107 | (S)-N-(4-(4-amino-1-methyl-7-(6- (4-methylpiperazin-1-yl)pyridin-3- yl)-1H-pyrazolo[4,3-c]pyridin-3-yl)- 2-(1-(4- fluorophenyl)ethoxy)phenyl)-1,1- difluoromethanesulfonamide | 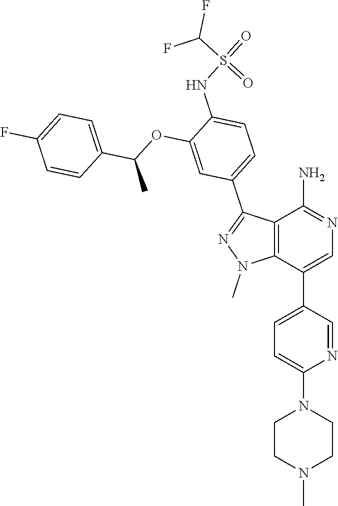 [SMILES: C[C@H](Oc1cc(-c2nn(C)c3c(-c4ccc(N5CCN(C)CC5)nc4)cnc(N)c23)ccc1NS(=O)(=O)C(F)F)c1ccc(F)cc1] | 75% | (Method A): 2.56 min, m/z: 667.2 [M + H]⁺. | (400 MHz, DMSO-d₆) 81.9 (s, 1H), 7.68 (d, J = 7.2 Hz, 1H), 7.55 (t, J = 6.4 Hz, 2H), 7.51 (s, 1H), 7.39 (d, J = 8.0 Hz, 1H), 7.16 (t, J = 8.8 Hz, 2H), 7.10-7.05 (m, 2H), 6.98 (d, J = 8.8 Hz, 1H), 6.87 (s, 0.5H), 6.74 (s, 0.2H), 5.77 (s, 2H), 5.65 (q, J = 6.0 Hz, 1H), 3.70 (s, 4H), 3.61 (s, 3H), 2.83 (s, 4H), 1.56 (d, J = 6.0 Hz, 3H), 1.23 (s, 3H). | 1-methyl-4-[5-(4,4,5,5- tetramethyl-1,3,2- dioxaborolan-2-yl)pyridin- 2-yl]piperazine |
| 110 | (S)-N-(4-(4-amino-1-methyl-7-(2- morpholinopyrimidin-5-yl)-1H- pyrazolo[4,3-c]pyridin-3-yl)-2-(1-(4- fluorophenyl)ethoxy)phenyl)-1,1- difluoromethanesulfonamide | 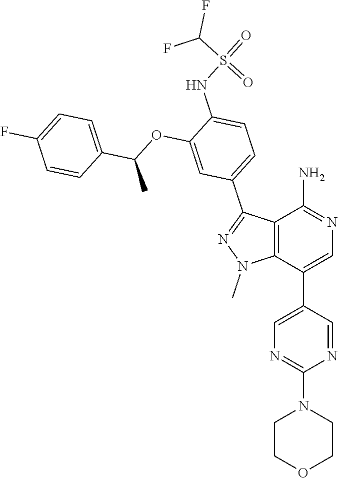 [SMILES: C[C@H](Oc1cc(-c2nn(C)c3c(-c4cnc(N5CCOCC5)nc4)cnc(N)c23)ccc1NS(=O)(=O)C(F)F)c1ccc(F)cc1] | 71% | (Method A): 3.05 min, m/z: 655.0 [M + H]⁺. | (400 MHz, DMSO-d₆) 10.77 (s, 1H), 8.50 (s, 2H), 7.58-7.55 (m, 3H), 7.40 (d, J = 8.0 Hz, 1H), 7.19-7.14 (m, 3H), 7.12 (s, 0.5H), 7.09 (s, 1H), 7.01 (s, 0.3H), 6.88 (s, 0.2H), 5.91 (s, 2H), 5.65 (q, J = 5.6 Hz, 1H), 3.75 (d, J = 4.8 Hz, 4H), 3.70 (d, J = 4.4 Hz, 4H), 3.68 (s, 3H), 1.58 (d, J = 6.4 Hz, 3H). | 4-[5-(4,4,5,5-tetramethyl- 1,3,2-dioxaborolan-2- yl)pyrimidin-2- yl]morpholine |
| 114 | (S)-N-(4-(4-amino-1-methyl-7-(2- (pyrrolidin-1-yl)pyrimidin-5-yl)-1H- pyrazolo[4,3-c]pyridin-3-yl)-2-(1-(4- fluorophenyl)ethoxy)phenyl)-1,1- difluoromethanesulfonamide | 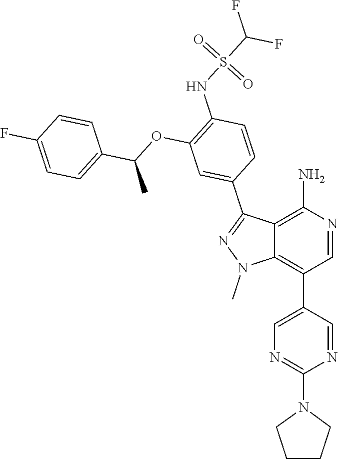 [SMILES: C[C@H](Oc1cc(-c2nn(C)c3c(-c4cnc(N5CCCC5)nc4)cnc(N)c23)ccc1NS(=O)(=O)C(F)F)c1ccc(F)cc1] | 49% | (Method A): 3.31 min, m/z: 639.0 [M + H]⁺. | (400 MHz, DMSO-d₆) 8.45, 7.60-7.56 (m), 7.42 (d, J = 8.0 Hz), 7.21-7.11 (m), 6.98 (t, J = 52.6 Hz), 6.14, 5.66 (q, J = 6.1 Hz), 3.69, 3.54 (t, J = 6.5 Hz), 1.99-1.95 (m), 1.59 (d, J = 6.3 Hz). | 2-(pyrrolidin-1-yl)-5- (4,4,5,5-tetramethyl-1,3,2- dioxaborolan-2- yl)pyrimidine |
| 106 | (S)-N-(4-(4-amino-7-(2- (dimethylamino)pyrimidin-5-yl)-1- methyl-1H-pyrazolo[4,3-c]pyridin- 3-yl)-2-(1-(4- fluorophenyl)ethoxy)phenyl)-1,1- difluoromethanesulfonamide | 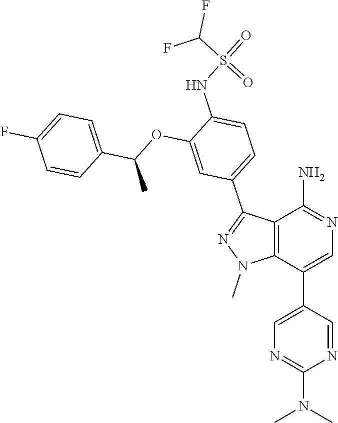 [SMILES: C[C@H](Oc1cc(-c2nn(C)c3c(-c4cnc(N(C)C)nc4)cnc(N)c23)ccc1NS(=O)(=O)C(F)F)c1ccc(F)cc1] | 47% | (Method A): 3.00 min, m/z: 613.0 [M + H]⁺. | (400 MHz, DMSO-d₆) 10.58 (s, 1H), 8.45 (s, 1H), 7.56 (q, J = 4 Hz, 2H), 7.52 (s, 1H), 7.40 (d, J = 8 Hz, 1H), 7.17 (t, J = 10 Hz, 2H), 7.10 (d, J = 8 Hz, 1H), 7.07 (s, 1H), 6.92 (t, J = 88 Hz, 1H), 5.77 (s, 2H), 5.65 (q, J = 20 Hz, 1H), 3.67 (s, 3H), 3.18 (s, 6H), 1.57 (d, J = 8 Hz, 3H) | N,N-dimethyl-5-(4,4,5,5- tetramethyl-1,3,2- dioxaborolan-2- yl)pyrimidin-2-amine |
| 138 | (S)-N-(4-(4-amino-1-methyl-7-(1- (2-(methylamino)pyrimidin-5-yl)- 1H-pyrazol-4-yl)-1H-pyrazolo[4,3- c]pyridin-3-yl)-2-(1-(4- fluorophenyl)ethoxy)phenyl)-1,1- difluoromethanesulfonamide | 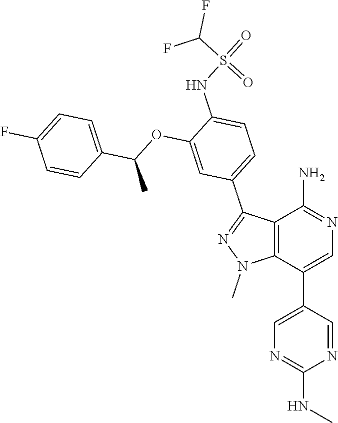 | 41% | (Method A), 2.92 min, m/z 665.1 [M + H]⁺. | (400 MHz, DMSO-d₆) 8.95 (s, 1H), 8.37 (s, 2H), 7.92 (s, 1H), 7.58-7.53 (m, 3H), 7.41 (d, J = 8 Hz, 1H), 7.23 (q, J = 5.2 Hz, 1H), 7.19-7.10 (m, 4H), 6.99 (t, J = 52 Hz, 1H), 6.39 (s, 1H), 5.78 (s, 2H), 5.75 (s, 1H), 5.65 (q, J = 5.2 Hz, 1H), 3.68 (s, 3H), 2.85 (d, J = 4 Hz, 3 H), 1.58 (d, J = 8 Hz, 3H). | N-methyl-5-(4,4,5,5- tetramethyl-1,3,2- dioxaborolan-2- yl)pyrimidin-2-amine |
| 115 | (S)-N-(4-(4-amino-1-methyl-7-(6- morpholinopyridin-3-yl)-1H- pyrazolo[4,3-c]pyridin-3-yl)-2-(1-(4- fluorophenyl)ethoxy)phenyl)-1,1- difluoromethanesulfonamide | 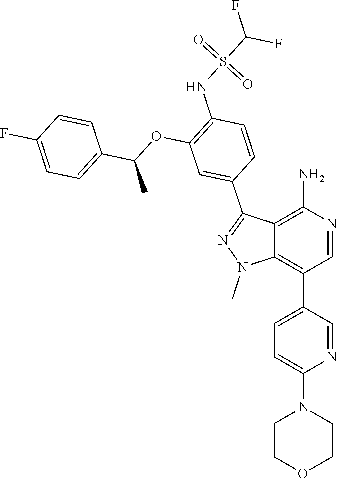 [SMILES: C[C@H](Oc1cc(-c2nn(C)c3c(-c4ccc(N5CCOCC5)nc4)cnc(N)c23)ccc1NS(=O)(=O)C(F)F)c1ccc(F)cc1] | 38% | (Method A): 3.07 min, m/z: 654.0 [M + H]⁺. | (400 MHz, DMSO-d₆) 8.19 (d, J = 1.9 Hz), 7.68 (dd, J = 8.7, 2.1 Hz), 7.60-7.54 (m), 7.41 (d, J = 8.0 Hz), 7.21-7.09 (m), 6.93 (d, J = 8.8 Hz), 5.76, 5.66 (d, J = 6.4 Hz), 3.76- 3.70 (m), 3.62, 3.53-3.49 (m), 1.58 (d, J = 6.1 Hz). | 4-[5-(4,4,5,5-tetramethyl- 1,3,2-dioxaborolan-2- yl)pyridin-2-yl]morpholine |
| 117 | (S)-N-(4-(4-amino-1-methyl-7-(6- (trifluoromethyl)pyridin-3-yl)-1H- pyrazolo[4,3-c]pyridin-3-yl)-2-(1-(4- fluorophenyl)ethoxy)phenyl)-1,1- difluoromethanesulfonamide/ | 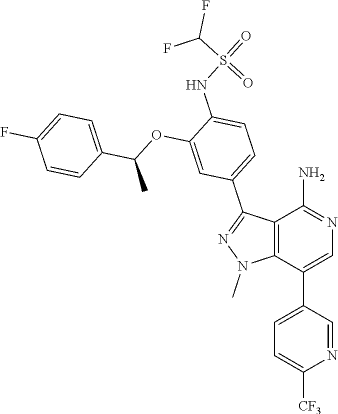 [SMILES: C[C@H](Oc1cc(-c2nn(C)c3c(-c4ccc(C(F)(F)F)nc4)cnc(N)c23)ccc1NS(=O)(=O)C(F)F)c1ccc(F)cc1] | 25% | (Method A): 3.44 min, m/z: 637.0 [M + H]⁺. | (400 MHz, DMSO-d₆) 8.90 (d, J = 2.1 Hz, 1H), 8.22 (dd, J = 8.1, 2.1 Hz, 1H), 7.99 (d, J = 8.1 Hz, 1H), 7.69 (s, 1H), 7.61- 7.53 (m, 2H), 7.42 (d, J = 8.0 Hz, 1H), 7.21-7.11 (m, 4H), 6.95 (d, J = 52.6 Hz, 1H), 5.96 (s, 2H), 5.65 (d, J = 6.4 Hz, 1H), 3.59 (s, 3H), 1.59 (d, J = 6.2 Hz, 3H). | 5-(4,4,5,5-tetramethyl- 1,3,2-dioxaborolan-2-yl)-2- (trifluoromethyl)pyridine |
| 160 | (S)-N-(4-(4-amino-1-methyl-7-(3- methyl-2-oxo-2,3-dihydro-1H- benzo[d]imidazol-5-yl)-1H- pyrazolo[4,3-c]pyridin-3-yl)-2-(1-(4- fluorophenyl)ethoxy)phenyl)-1,1- difluoromethanesulfonamide | 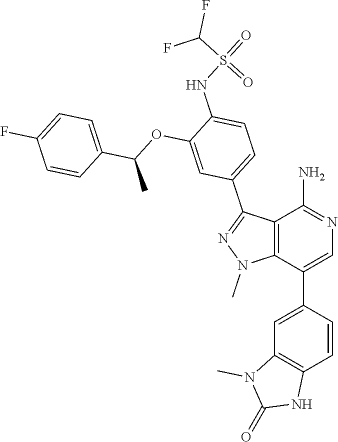 [SMILES: C[C@H](Oc1cc(-c2nn(C)c3c(-c4ccc5[nH]c(=O)n(C)c5c4)cnc(N)c23)ccc1NS(=O)(=O)C(F)F)c1ccc(F)cc1] | | (Method A): 2.62 min, m/z: 638.2 [M + H]⁺. | (400 MHz, MDSO-d₆) 10.94 (s, 1H), 7.58-7.55 (m, 3H), 7.41 (d, J = 8 Hz, 1H), 7.19-7.06 (m, 6H), 7.01 (s, 1H), 6.99 (t, J = 52 Hz, 1H), 5.78 (s, 2H), 5.65 (q, J = 6.8 Hz, 1H), 3.51 (s, 3H), 3.33 (s, 3H), 1.58 (d, J = 8 Hz, 3H) | 1-methyl-6-(4,4,5,5- tetramethyl-1,3,2- dioxaborolan-2-yl)-1,3- dihydro-2H- benzo[d]imidazol-2-one |
| 161 | (S)-N-(4-(4-amino-7-(1,3-dimethyl- 2-oxo-2,3-dihydro-1H- benzo[d]imidazol-5-yl)-1-methyl- 1H-pyrazolo[4,3-c]pyridin-3-yl)-2- (1-(4-fluorophenyl)ethoxy)phenyl)- 1,1-difluoromethanesulfonamide | 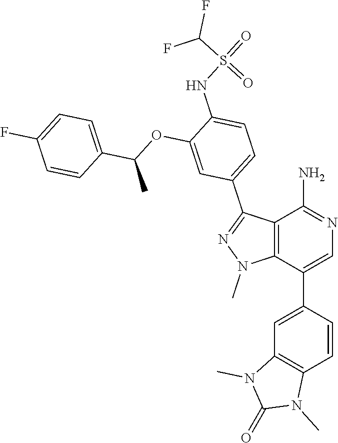 [SMILES: C[C@H](Oc1cc(-c2nn(C)c3c(-c4ccc5c(c4)n(C)c(=O)n5C)cnc(N)c23)ccc1NS(=O)(=O)C(F)F)c1ccc(F)cc1] | | (Method A): 2.72 min, m/z: 652.2 [M + H]⁺. | (400 MHz, DMSO-d₆) 7.58-7.55 (m, 3H), 7.41 (d, J = 8 Hz, 1H), 7.25- 7.11 (m, 7H), 6.99 (t, J = 52 Hz, 1H), 5.79 (s, 2H), 5.65 (q, J = 6.8 Hz, 1H), 3.52 (s, 3H), 3.38 (s, 3H), 3.35 (s, 3H), 1.58 (d, J = 4 Hz, 3H) | 1,3-dimethyl-5-(4,4,5,5- tetramethyl-1,3,2- dioxaborolan-2-yl)-1,3- dihydro-2H- benzo[d]imidazol-2-one |
| 165 | (S)-N-(4-(4-amino-1-methyl-7-(1- methyl-2-oxoindolin-5-yl)-1H- pyrazolo[4,3-c]pyridin-3-yl)-2-(1-(4- fluorophenyl)ethoxy)phenyl)-1,1- difluoromethanesulfonamide | 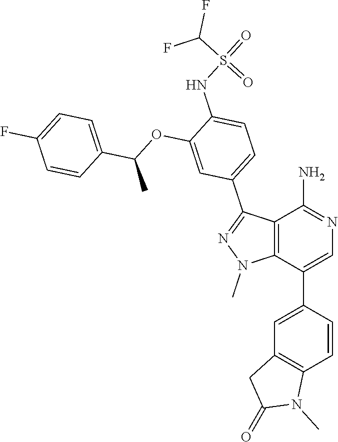 [SMILES: C[C@H](Oc1cc(-c2nn(C)c3c(-c4ccc5c(c4)CC(=O)N5C)cnc(N)c23)ccc1NS(=O)(=O)C(F)F)c1ccc(F)cc1] | | (Method A): 2.76 min, m/z: 637.2 [M + H]⁺. | (400 MHz, DMSO-d₆) 10.66 (s, 1H), 7.59-7.54 (m, 3H), 7.41 (d, J = 8 Hz, 1H), 7.34 (d, J = 8 Hz, 2H), 7.20-7.13 (m, 4H), 7.07 (d, J = 8 Hz, 1H), 7.01 (t, J = 52 Hz, 1H), 5.79 (s, 2H), 5.66 (q, J = 6.8 Hz, 1H), 3.62 (s, 2H), 3.56 (s, 3H), 3.17 (s, 3H), 1.58 (d, J = 4 Hz, 3H) | 1-methyl-5-(4,4,5,5- tetramethyl-1,3,2- dioxaborolan-2-yl)indoline-2- one |
| 166 | (S)-N-(4-(4-amino-1-methyl-7-(1- methyl-1H-indazol-5-yl)-1H- pyrazolo[4,3-c]pyridin-3-yl)-2-(1-(4- fluorophenyl)ethoxy)phenyl)-1,1- difluoromethanesulfonamide | 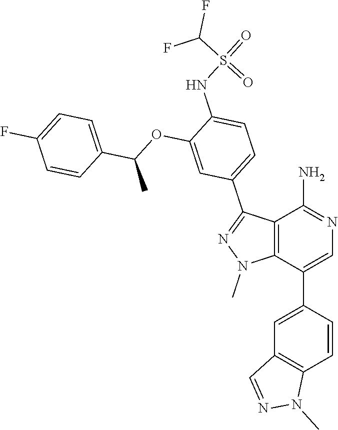 [SMILES: C[C@H](Oc1cc(-c2nn(C)c3c(-c4ccc5c(cnn5C)c4)cnc(N)c23)ccc1NS(=O)(=O)C(F)F)c1ccc(F)cc1] | | (Method A): 2.74 min, m/z: 622.2 [M + H]⁺. | (400 MHz, DMSO-d₆) 10.66 (s, 1H), 8.10 (s, 1H), 7.80 (s, 1H), 7.73 (d, J = 12 Hz, 1H), 7.59-7.56 (m, 3H), 7.48 (d, J = 8 Hz, 1H), 7.42 (d, J = 8 Hz, 1H), 7.20-7.14 (m, 4H), 7.01 (t, J = 52 Hz, 1H), 5.81 (s, 2H), 5.67 (q, J = 5.2 Hz, 1H), 4.10 (s, 3H), 3.49 (s, 3H), 1.59 (d, J = 4 Hz, 3H) | 1-methyl-5-(4,4,5,5- tetramethyl-1,3,2- dioxaborolan-2-yl)-1H- indazole |
| 116 | (S)-N-(4-(4-amino-1-methyl-7-(1- (2,2,2-trifluoroethyl)-1H-pyrazol-4- yl)-1H-pyrazolo[4,3-c]pyridin-3-yl)- 2-(1-(4- fluorophenyl)ethoxy)phenyl)-1,1- difluoromethanesulfonamide | 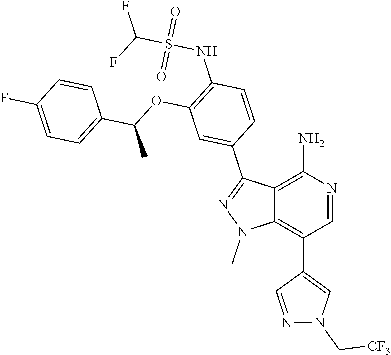 [SMILES: C[C@H](Oc1cc(-c2nn(C)c3c(-c4cnn(CC(F)(F)F)c4)cnc(N)c23)ccc1NS(=O)(=O)C(F)F)c1ccc(F)cc1] | 39% | (Method A): 3.17 min, m/z: 640.0 [M + H]⁺. | (400 MHz, DMSO-d₆) 8.07, 7.77, 7.57, 7.41 (d, J = 8.0 Hz), 7.20-7.10 (m), 6.94 (d, J = 52.6 Hz), 5.78, 5.66 (d, J = 6.3 Hz), 5.21 (q, J = 9.1 Hz), 3.68, 1.58 (d, J = 6.2 Hz). | 4-(4,4,5,5-tetramethyl- 1,3,2-dioxaborolan-2-yl)-1- (2,2,2-trifluoroethyl)-1H- pyrazole |
| 113 | (S)-N-(4-(4-amino-1-methyl-7-(1- (3,3,3-trifluoropropyl)-1H-pyrazol- 4-yl)-1H-pyrazolo[4,3-c]pyridin-3- yl)-2-(1-(4- fluorophenyl)ethoxy)phenyl)-1,1- difluoromethanesulfonamide | 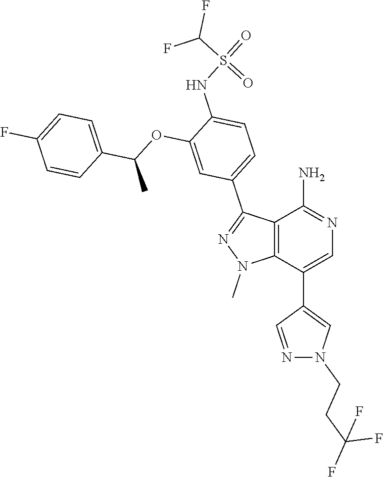 [SMILES: C[C@H](Oc1cc(-c2nn(C)c3c(-c4cnn(CCC(F)(F)F)c4)cnc(N)c23)ccc1NS(=O)(=O)C(F)F)c1ccc(F)cc1] | | (Method A): 3.27 min, m/z: 654.1 [M + H]⁺. | (400 MHz, DMSO-d₆) 8.02 (s, 1H), 7.67 (s, 1H), 7.59 (dd, J = 8.2, 5.8 Hz, 2H), 7.55 (s, 1H), 7.43 (d, J = 8.0 Hz, 1H), 7.18 (dd, J = 19.6, 10.6 Hz, 4H), 6.95 (d, J = 52.7 Hz, 1H), 5.76 (s, 2H), 5.68 (q, J = 6.2 Hz, 1H), 4.48 (t, J = 6.7 Hz, 2H), 3.71 (s, 3H), 3.02-2.89 (m, 2H), 1.61 (d, J = 6.2 Hz, 3H). | 4-(4,4,5,5-tetramethyl- 1,3,2-dioxaborolan-2-yl)-1- (3,3,3-trifluoropropyl)-1H- pyrazole |
| 134 | (S)-N-(4-(4-amino-1-methyl-7-(1- (trifluoromethyl)-1H-pyrazol-4-yl)- 1H-pyrazolo[4,3-c]pyridin-3-yl)-2- (1-(4-fluorophenyl)ethoxy)phenyl)- 1,1-difluoromethanesulfonamide | 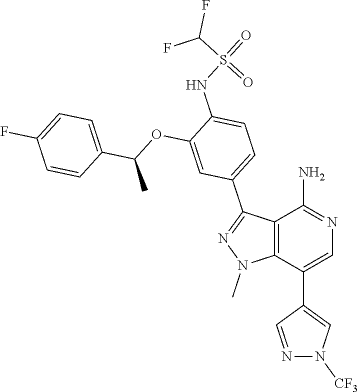 [SMILES: C[C@H](Oc1cc(-c2nn(C)c3c(-c4cnn(C(F)(F)F)c4)cnc(N)c23)ccc1NS(=O)(=O)C(F)F)c1ccc(F)cc1] | | (Method A): 3.37 min, m/z: 626.0 [M + H]⁺. | (400 MHz, DMSO-d₆) 8.72 (s, 1H), 8.21 (s, 1H), 7.64 (d, J = 1.5 Hz, 1H), 7.57 (dd, J = 8.4, 5.6 Hz, 2H), 7.43-7.38 (m, 1H), 7.21-7.08 (m, 4H), 6.96 (d, J = 52.6 Hz, 1H), 5.86 (s, 2H), 5.65 (q, J = 6.2 Hz, 1H), 3.72 (d, J = 1.5 Hz, 3H), 1.58 (d, J = 6.2 Hz, 3H) | 4-(4,4,5,5-tetramethyl- 1,3,2-dioxaborolan-2-yl)-1- (trifluoromethyl)-1H- pyrazole |
| 123 | (S)-N-(4-(4-amino-7-(1- (difluoromethyl)-1H-pyrazol-4-yl)- 1-methyl-1H-pyrazolo[4,3- c]pyridin-3-yl)-2-(1-(4- fluorophenyl)ethoxy)phenyl)-1,1- difluoromethanesulfonamide | 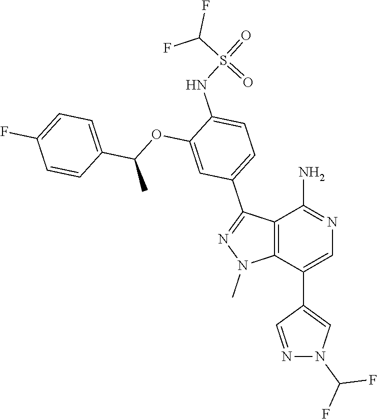 [SMILES: C[C@H](Oc1cc(-c2nn(C)c3c(-c4cnn(C(F)F)c4)cnc(N)c23)ccc1NS(=O)(=O)C(F)F)c1ccc(F)cc1] | 30% | (Method A): 3.39 min, m/z: 608.0 [M + H]⁺. | (400 MHz, DMSO-d₆) 8.46 (s, 1H), 8.01 (s, 1H), 7.79 (d, J = 59.1 Hz, 1H), 7.60 (s, 3H), 7.41 (d, J = 8.0 Hz, 1H), 7.20-7.09 (m, 4H), 6.94 (d, J = 52.7 Hz, 1H), 5.81 (s, 2H), 5.65 (q, J = 6.2 Hz, 1H), 3.70 (s, 3H), 1.58 (d, J = 6.2 Hz, 3H). | 1-(Difluoromethyl)-4- (4,4,5,5-tetramethyl-1,3,2- dioxaborolan-2-yl)-1H- pyrazole |
| 142 | N-(4-(4-amino-1-methyl-7-(1- (1,1,1-trifluoropropan-2-yl)-1H- pyrazol-4-yl)-1H-pyrazolo[4,3- c]pyridin-3-yl)-2-((S)-1-(4- fluorophenyl)ethoxy)phenyl)-1,1- difluoromethanesulfonamide | 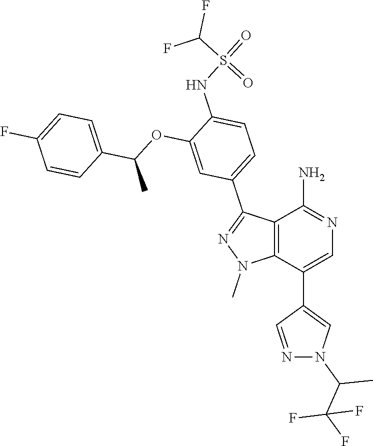 [SMILES: CC(n1cc(-c2cnc(N)c3c(-c4ccc(NS(=O)(=O)C(F)F)c(O[C@@H](C)c5ccc(F)cc5)c4)nn(C)c23)cn1)C(F)(F)F] | | (Method A): 3.31 min, m/z: 654.1 [M + H]⁺. | (400 MHz, DMSO-d₆) 8.16 (s, 1H), 7.76 (s, 2H), 7.59-7.55 (m, 3H), 7.41 (d, J = 8 Hz, 1H), 7.19- 7.11 (m, 4H), 7.10 (t, J = 32 Hz, 1H), 5.86 (s, 2H), 5.66 (q, J = 6.8 Hz, 1H), 5.49-5.41 (m, 1H), 3.68 (s, 3H), 1.74 (d, J = 8 Hz, 3H), 1.58 (d, J = 4 Hz, 3H). | 4-(4,4,5,5-tetramethyl- 1,3,2-dioxaborolan-2-yl)-1- (1,1,1-trifluoropropan-2-yl)- 1H-pyrazole |
| 97 | (S)-N-(4-(4-amino-7-(1-(1-hydroxy- 2-methylpropan-2-yl)-1H-pyrazol- 4-yl)-1-methyl-1H-pyrazolo[4,3- c]pyridin-3-yl)-2-(1-(4- fluorophenyl)ethoxy)phenyl)-1,1- difluoromethanesulfonamide | 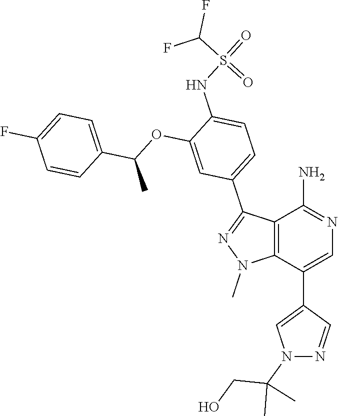 [SMILES: C[C@H](Oc1cc(-c2nn(C)c3c(-c4cnn(C(C)(C)CO)c4)cnc(N)c23)ccc1NS(=O)(=O)C(F)F)c1ccc(F)cc1] | 53% | (Method A): 3.07 min, m/z: 630.3 [M + H]⁺. | (400 MHz, DMSO-d₆) 10.69 (s, 1H), 7.97 (s, 1H), 7.59-7.55 (m, 4H), 7.41 (d, J = 8 Hz, 1H), 7.19-7.10 (m, 4H), 6.99 (t, J = 52 Hz, 1H), 5.73 (s, 2H), 5.66 (q, J = 8 Hz, 1H), 4.97 (t, J = 4 Hz, 1H), 3.69 (s, 3H), 3.63 (d, J = 4 Hz, 2 H), 1.59 (d, J = 4 Hz, 3H), 1.52 (s, 6H). | 2-methyl-2-[4-(4,4,5,5- tetramethyl-1,3,2- dioxaborolan-2-yl)-1H- pyrazol-1-yl]propan-1-ol |
| 75 | (S)-N-(4-(4-amino-7-(1-(2-(3,3- dimethylazetidin-1-yl)ethyl)-1H- pyrazol-4-yl)-1-methyl-1H- pyrazolo[4,3-c]pyridin-3-yl)-2-(1-(4- fluorophenyl)ethoxy)phenyl)-1,1- difluoromethanesulfonamide | 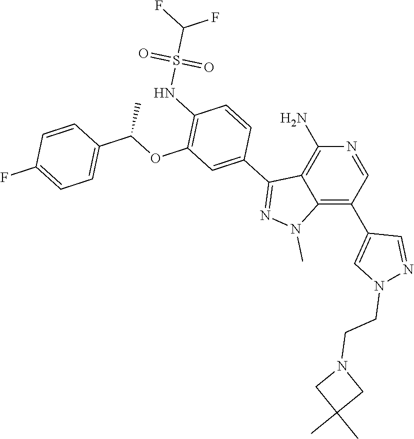 [SMILES: C[C@H](Oc1cc(-c2nn(C)c3c(-c4cnn(CCN5CC(C)(C)C5)c4)cnc(N)c23)ccc1NS(=O)(=O)C(F)F)c1ccc(F)cc1] | 52% | (Method A): 2.58 min, m/z: 669.2 [M + H]⁺. | (400 MHz, DMSO-d₆) 7.91 (s, 1H), 7.61 (s, 1H), 7.56-7.51 (m, 3H), 7.38 (d, J = 8.4 Hz, 1H), 7.15 (t, J = 9.2 Hz, 2H), 7.05 (dd, J = 1.6, 8.0 Hz, 1H), 7.01 (s, 1H), 6.76 (t, J = 53.6 Hz, 1H), 5.66-5.63 (m, 3H), 4.18 (t, J = 6.0 Hz, 2H), 3.71 (s, 3H), 3.11 (s, 4H), 3.07-3.04 (m, 2H), 1.56 (d, J = 6.4 Hz, 3H), 1.17 (s, 6H). | Intermediate C3 |
| 82 | (S)-N-(4-(4-amino-1-methyl-7-(1- (2-(methylamino)ethyl)-1H- pyrazol-4-yl)-1H-pyrazolo[4,3- c]pyridin-3-yl)-2-(1-(4- fluorophenyl)ethoxy)phenyl)-1,1- difluoromethanesulfonamide | 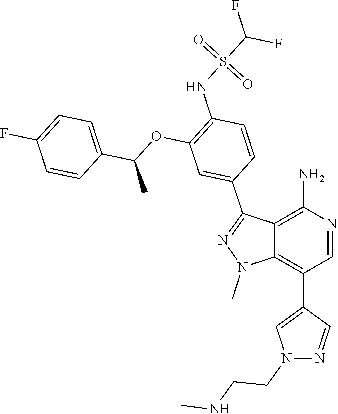 [SMILES: CNCCn1cc(-c2cnc(N)c3c(-c4ccc(NS(=O)(=O)C(F)F)c(O[C@@H](C)c5ccc(F)cc5)c4)nn(C)c23)cn1] | 19% | (Method A): 2.01 min, m/z: 615.3 [M + H]⁺. | (400 MHz, DMSO-d₆) 7.95 (s, 1H), 7.68 (s, 1H), 7.57-7.49 (m, 3 H), 7.37- 7.35 (m, 1H), 7.16-7.11 (m, 2H), 6.96-6.93 (m, 1H), 6.88-6.87 (m, 1H), 6.57-6.30 (m, 1H), 5.68- 5.64 (m, 3H), 4.40-4.37 (m, 2H), 3.70 (s, 3H), 3.29-3.26 (m, 2H), 2.52 (s, 3H), 1.53-1.51 (d, J = 6.4 Hz, 2H). | Intermediate C4 |
| 100 | (S)-N-(4-(4-amino-7-(1-(2- (dimethylamino)ethyl)-1H-pyrazol- 4-yl)-1-methyl-1H-pyrazolo[4,3- c]pyridin-3-yl)-2-(1-(4- fluorophenyl)ethoxy)phenyl)-1,1- difluoromethanesulfonamide | 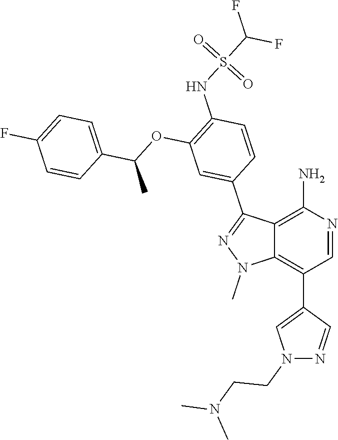 [SMILES: C[C@H](Oc1cc(-c2nn(C)c3c(-c4cnn(CCN(C)C)c4)cnc(N)c23)ccc1NS(=O)(=O)C(F)F)c1ccc(F)cc1] | 33% | (Method A): 1.99 min, m/z: 629.2 [M + H]⁺. | (400 MHz, DMSO-d₆) 7.93 (s, 1H), 7.59 (s, 1 H), 7.57-7.52 (m, 3H), 7.42-7.38 (m, 1H), 7.19- 7.14 (m, 2H), 7.13-7.04 (m, 2H), 6.96-6.69 (m, 1H), 5.75-5.63 (m, 3H), 4.31-4.28 (m, 2H), 3.70 (s, 3 H), 2.85-2.82 (m, 2 H), 2.29 (s, 6 H), 1.57- 1.56 (d, J = 6.4 Hz, 3 H). | Intermediate C5 |
| 73 | N-(4-(4-amino-1-methyl-7-(1-(8- methyl-8-azabicyclo[3.2.1]octan-3- yl)-1H-pyrazol-4-yl)-1H- pyrazolo[4,3-c]pyridin-3-yl)-2-((S)- 1-(4-fluorophenyl)ethoxy)phenyl)- 1,1-difluoromethanesulfonamide | 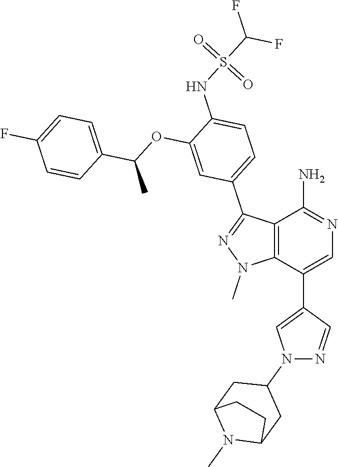 [SMILES: C[C@H](Oc1cc(-c2nn(C)c3c(-c4cnn(C5CC6CCC(C5)N6C)c4)cnc(N)c23)ccc1NS(=O)(=O)C(F)F)c1ccc(F)cc1] | 24% | (Method A): 2.80 min, m/z: 681.5 [M + H]⁺. | (400 MHz, DMSO-d₆) 10.96 (s, 1H), 8.29 (s, 1H), 8.00 (d, J = 47.3 Hz, 1H), 7.80 (s, 1H), 7.70 (s, 2H), 7.60-7.53 (m, 3H), 7.40 (d, J = 8.1 Hz, 1H), 7.13 (dt, J = 27.3, 9.1 Hz, 4H), 5.65 (q, J = 6.2 Hz, 1H), 3.72 (s, 3H), 3.06- 2.93 (m, 2H), 2.82 (d, J = 20.6 Hz, 3H), 2.71-2.56 (m, 4H), 2.09-2.00 (m, 2H), 1.73 (t, J = 7.1 Hz, 2H), 1.57 (d, J = 6.2 Hz, 3H). | Intermediate C6 |
| 126 | (S)-N-(4-(4-amino-1-methyl-7-(1- methyl-1H-pyrrol-3-yl)-1H- pyrazolo[4,3-c]pyridin-3-yl)-2-(1-(4- fluorophenyl)ethoxy)phenyl)-1,1- difluoromethanesulfonamide | 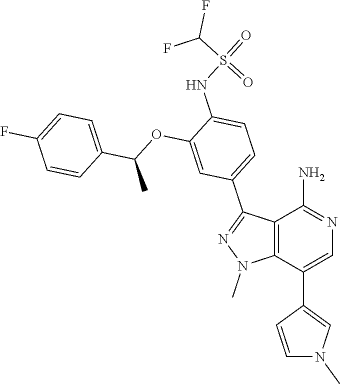 [SMILES: C[C@H](Oc1cc(-c2nn(C)c3c(-c4ccn(C)c4)cnc(N)c23)ccc1NS(=O)(=O)C(F)F)c1ccc(F)cc1] | 3% | (Method A): 3.21 min, m/z: 571.1 [M + H]⁺. | (400 MHz, CDCl₃) 7.66 (d, J = 8.2 Hz, 1H), 7.53 (s, 1H), 7.33-7.28 (m, 2H), 7.16 (dd, J = 8.2, 1.8 Hz, 1H), 7.03 (d, J = 1.8 Hz, 1H), 6.98 (t, J = 8.6 Hz, 2H), 6.65 (dt, J = 15.5, 2.2 Hz, 2H), 6.33 (t, J = 53.8 Hz, 1H), 6.16- 6.13 (m, 1H), 5.92 (s, 1H), 5.44 (q, J = 6.4 Hz, 1H), 3.81 (s, 3H), 3.71 (s, 3H), 1.64 (d, J = 6.4 Hz, 3H). | 1-methyl-3-(4,4,5,5- tetramethyl-1,3,2- dioxaborolan-2-yl)-1H- pyrrole |
| 83 | (S)-N-(4-(4-amino-7-(5-cyano-1- methyl-1H-pyrrol-3-yl)-1-methyl- 1H-pyrazolo[4,3-c]pyridin-3-yl)-2- (1-(4-fluorophenyl)ethoxy)phenyl)- 1,1-difluoromethanesulfonamide | 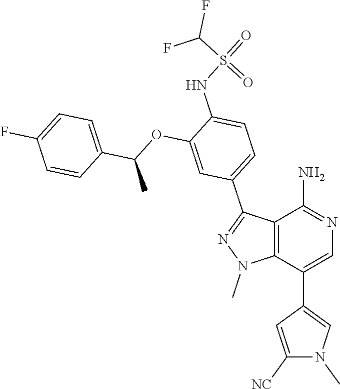 [SMILES: C[C@H](Oc1cc(-c2nn(C)c3c(-c4cc(C#N)n(C)c4)cnc(N)c23)ccc1NS(=O)(=O)C(F)F)c1ccc(F)cc1] | 3% | (Method C): 4.08 min, m/z: 596.0 [M + H]⁺. | (400 MHz, DMSO-d₆) 7.63-7.49 (m, 3H), 7.44- 7.33 (m, 2H), 7.15 (td, J = 15.5, 13.5, 6.9 Hz, 5H), 6.95 (d, J = 52.6 Hz, 1H), 5.77 (s, 2H), 5.66 (d, J = 6.4 Hz, 1H), 3.83 (s, 3H), 3.72 (s, 3H), 1.58 (d, J = 6.2 Hz, 3H). | Intermediate C7 |
| 98 | (S)-N-(4-(4-amino-7-(1-(3- (cyanomethyl)oxetan-3-yl)-1H- pyrazol-4-yl)-1-methyl-1H- pyrazolo[4,3-c]pyridin-3-yl)-2-(1-(4- fluorophenyl)ethoxy)phenyl)-1,1- difluoromethanesulfonamide | 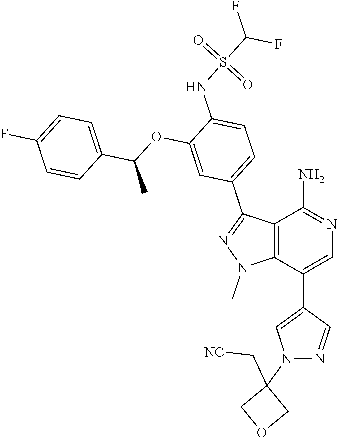 [SMILES: C[C@H](Oc1cc(-c2nn(C)c3c(-c4cnn(C5(CC#N)COC5)c4)cnc(N)c23)ccc1NS(=O)(=O)C(F)F)c1ccc(F)cc1] | 9% | (Method A): 3.04 min, m/z: 653.0 [M + H]⁺. | (400 MHz, DMSO-d₆) 8.31 (s, 1H), 7.82 (s, 1H), 7.60 (s, 1H), 7.57 (dd, J = 8.6, 5.7 Hz, 2H), 7.41 (d, J = 8.0 Hz, 1H), 7.19- 7.11 (m, 4H), 7.01 (t, J = 52 Hz, 1H), 5.78 (s, 2H), 5.66 (q, J = 6.3 Hz, 1H), 5.08 (d, J = 7.2 Hz, 2H), 4.82 (d, J = 7.2 Hz, 2H), 3.71 (s, 3H), 3.66 (s, 2H), 1.59 (d, J = 6.2 Hz, 3H). | Intermediate C8 |
| 101 | N-(4-(4-amino-7-(1-(1- (isopropyl(methyl)amino)propan-2- yl)-1H-pyrazol-4-yl)-1-methyl-1H- pyrazolo[4,3-c]pyridin-3-yl)-2-((S)- 1-(4-fluorophenyl)ethoxy)phenyl)- 1,1-difluoromethanesulfonamide | 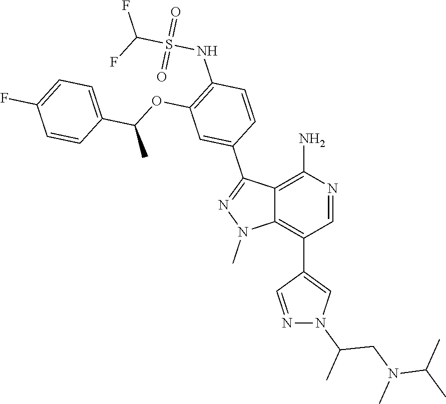 [SMILES: CC(C)N(C)CC(C)n1cc(-c2cnc(N)c3c(-c4ccc(NS(=O)(=O)C(F)F)c(O[C@@H](C)c5ccc(F)cc5)c4)nn(C)c23)cn1] | 46% | (Method C): 2.16 min, m/z: 671.3 [M + H]⁺. | (400 MHz, DMSO-d₆) 7.93 (s, 1H), 7.61-7.53 (m, 4H), 7.40-7.38 (m, 1H), 7.18-7.06 (m, 3 H), 7.02-6.56 (m, 1H), 5.67- 5.63 (m, 3H), 4.54-4.49 (m, 1H), 3.70 (s, 3 H), 2.83-2.67 (m, 3 H), 2.20 (s, 3 H), 1.58-1.56 (d, J = 6.4 Hz, 3 H), 1.46-1.44 (d, J = 6.8 Hz, 3 H), 0.95- 0.84 (m, 6 H). | Intermediate C9 |
| 96 | (S)-N-(4-(4-amino-7-(1-(1- cyanocyclopropyl)-1H-pyrazol-4- yl)-1-methyl-1H-pyrazolo[4,3- c]pyridin-3-yl)-2-(1-(4- fluorophenyl)ethoxy)phenyl)-1,1- difluoromethanesulfonamide | 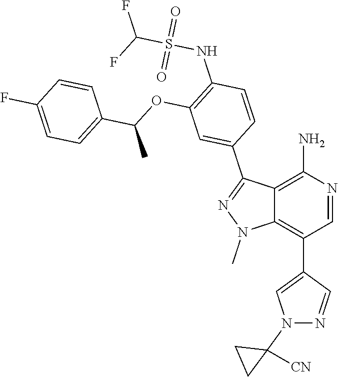 [SMILES: C[C@H](Oc1cc(-c2nn(C)c3c(-c4cnn(C5(C#N)CC5)c4)cnc(N)c23)ccc1NS(=O)(=O)C(F)F)c1ccc(F)cc1] | 43% | (Method C): 2.94 min, m/z: 623.3 [M + H]⁺. | (400 MHz, DMSO-d₆) 10.59 (brs, 1H), 8.23 (s, 1H), 7.79 (s, 1H), 7.59- 7.55 (m, 3H), 7.41 (d, J = 7.6 Hz, 1H), 7.19-6.88 (m, 5H), 5.77 (brs, 2H), 5.66 (q, J = 12.8, 6.4 Hz, 1H), 3.70 (s, 3H), 1.96- 1.93 (m, 2H), 1.90-1.86 (m, 2H), 1.58 (d, J = 6.0 Hz, 3H). | Intermediate C10 |
| 103 | (S)-N-(4-(4-amino-1-methyl-7-(1- (1-(methyl-d3)piperidin-4-yl)-1H- pyrazol-4-yl)-1H-pyrazolo[4,3- c]pyridin-3-yl)-2-(1-(4- fluorophenyl)ethoxy)phenyl)-1,1- difluoromethanesulfonamide | 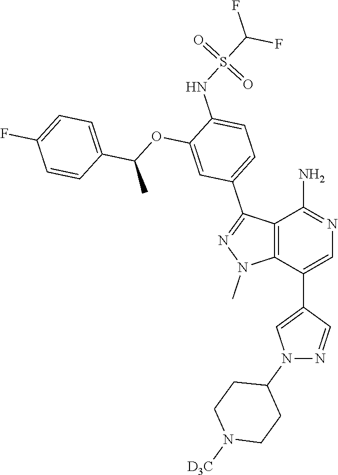 [SMILES: [2H]C([2H])([2H])N1CCC(n2cc(-c3cnc(N)c4c(-c5ccc(NS(=O)(=O)C(F)F)c(O[C@@H](C)c6ccc(F)cc6)c5)nn(C)c34)cn2)CC1] | 19% | (Method A): 2.54 min, m/z: 658.1 [M + H]⁺. | (400 MHz, DMSO-d₆) 7.97 (s, 1H), 7.61 (s, 1H), 7.55-7.52 (m, 3H), 7.37 (d, J = 8 Hz, 1H), 7.15 (t, J = 8.8 Hz, 2H), 7.02 (dd, J = 1.2, 8.4 Hz, 2H), 7.98 (s, 1H), 6.70 (t, J = 53.6 Hz, 1H), 5.67-5.62 (m, 3H), 4.33-4.26 (m, 1H), 3.67 (s, 3 H), 3.13-3.10 (m, 3H), 2.11-2.00 (m, 5H), 1.54 (d, J = 5.6 Hz, 3H). | Intermediate C11 |
| 93 | N-(4-(4-amino-1-methyl-7-(1-(1- methylpyrrolidin-3-yl)-1H-pyrazol- 4-yl)-1H-pyrazolo[4,3-c]pyridin-3- yl)-2-((S)-1-(4- fluorophenyl)ethoxy)phenyl)-1,1- difluoromethanesulfonamide | 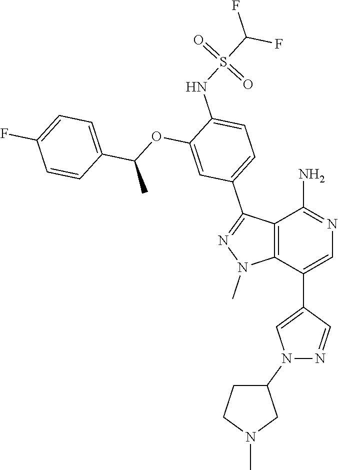 [SMILES: C[C@H](Oc1cc(-c2nn(C)c3c(-c4cnn(C5CCN(C)C5)c4)cnc(N)c23)ccc1NS(=O)(=O)C(F)F)c1ccc(F)cc1] | 25% | (Method A): 2.79 min, m/z: 641.3 [M + H]⁺. | (400 MHz, DMSO-d₆) 8.03 (s, 1H), 7.66 (s, 1H), 7.57-7.52 (m, 3H), 7.39 (d, J = 8.1 Hz, 1H), 7.16 (t, J = 8.9 Hz, 2H), 7.07 (dd, J = 8.1, 1.7 Hz, 1H), 7.03 (d, J = 1.5 Hz, 1H), 6.84 (t, J = 53.2 Hz, 1H), 5.71 (s, 2H), 5.65 (q, J = 6.2 Hz, 1H), 5.10 (tt, J = 8.8, 4.6 Hz, 1H), 3.69 (s, 4H), 3.33-3.27 (m, 3H), 3.20 (d, J = 4.2 Hz, 1H), 3.14 (d, J = 16.4 Hz, 3H), 2.98-2.88 (m, 2H), 2.58 (s, 3H), 1.56 (d, J = 6.3 Hz, 3H). | Intermediate C16 |
| 76 | (S)-N-(4-(4-amino-7-(1-(1- isopropylazetidin-3-yl)-1H-pyrazol- 4-yl)-1-methyl-1H-pyrazolo[4,3- c]pyridin-3-yl)-2-(1-(4- fluorophenyl)ethoxy)phenyl)-1,1- difluoromethanesulfonamide | 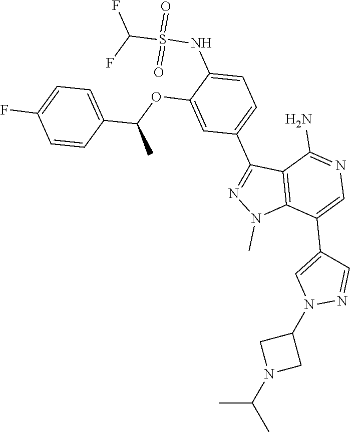 [SMILES: CC(C)N1CC(n2cc(-c3cnc(N)c4c(-c5ccc(NS(=O)(=O)C(F)F)c(O[C@@H](C)c6ccc(F)cc6)c5)nn(C)c34)cn2)C1] | 21% | (Method A): 3.10 min, m/z: 655.1 [M + H]⁺. | (400 MHz, DMSO-d₆) 8.13 (s, 1H), 7.79 (s, 1H), 7.58-7.54 (m, 3H), 7.40 (d, J = 8.0 Hz, 1H), 7.19- 6.82 (m, 5H), 5.74 (s, 2H), 5.67-5.63 (m, 1H), 5.26-5.19 (m, 1H), 4.27- 4.22 (br, 2H), 4.06 (br, 2H), 3.72 (s, 3H), 3.19- 3.13 (m, 1H), 1.58 (d, J = 6.4 Hz, 3H), 1.11 (d, J = 6.4 Hz, 6H). | Intermediate C17 |
| 92 | (S)-N-(4-(4-amino-1-methyl-7-(1- (1-methylazetidin-3-yl)-1H-pyrazol- 4-yl)-1H-pyrazolo[4,3-c]pyridin-3- yl)-2-(1-(4- fluorophenyl)ethoxy)phenyl)-1,1- difluoromethanesulfonamide | 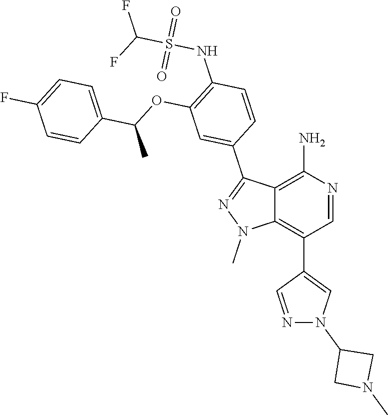 [SMILES: C[C@H](Oc1cc(-c2nn(C)c3c(-c4cnn(C5CN(C)C5)c4)cnc(N)c23)ccc1NS(=O)(=O)C(F)F)c1ccc(F)cc1] | 18% | (Method A): 3.05 min, m/z: 627.0 [M + H]⁺. | (400 MHz, MeOD-d₄) 7.94 (s, 1H), 7.69 (s, 1H), 7.54-7.45 (m, 3H), 7.36 (d, J = 8.0 Hz, 1H), 7.08- 6.99 (m, 3H), 6.90 (d, J = 1.8 Hz, 1H), 6.36 (t, J = 54.8 Hz, 1H), 5.51 (q, J = 6.3 Hz, 1H), 5.09 (p, J = 7.2 Hz, 1H), 3.90 (td, J = 7.3, 1.8 Hz, 2H), 3.72 (s, 3H), 3.67 (dd, J = 8.9, 7.0 Hz, 2H), 2.49 (s, 3H), 1.65 (d, J = 6.4 Hz, 3H). | Intermediate C18 |
| 77 | (S)-N-(4-(4-amino-1-methyl-7-(1- (1-(2,2,2-trifluoroethyl)azetidin-3- yl)-1H-pyrazol-4-yl)-1H- pyrazolo[4,3-c]pyridin-3-yl)-2-(1-(4- fluorophenyl)ethoxy)phenyl)-1,1- difluoromethanesulfonamide | 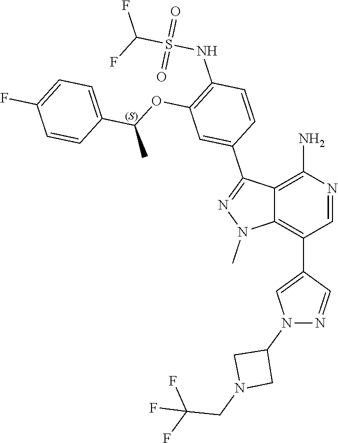 | 33% | (Method C): 3.00 min, m/z: 695.1 [M + H]⁺. | (400 MHz, DMSO-d₆) 10.62 (s, 1H), 8.11 (s, 1H), 7.7 (s, 1H), 7.59- 7.55 (m, 3H), 7.40 (d, J = 8.0 Hz, 1H), 7.19-7.11 (m, 4 H), 7.00-6.87 (m, 1H), 5.75 (s, 2H), 5.67- 5.63 (m, 1H), 5.17-5.09 (m, 1H), 3.92 (t, J = 7.6 Hz, 2H), 3.74-3.71 (m, 5H), 3.40-3.35 (m, 2H), 1.58 (d, J = 6.4 Hz, 3H). | Intermediate C19 |
| 81 | 2-(4-(4-amino-3-(4- ((difluoromethyl)sulfonamido)-3- ((S)-1-(4- fluorophenyl)ethoxy)phenyl)-1- methyl-1H-pyrazolo[4,3-c]pyridin- 7-yl)-1H-pyrazol-1-yl)-N-isopropyl- N-methylpropanamide | 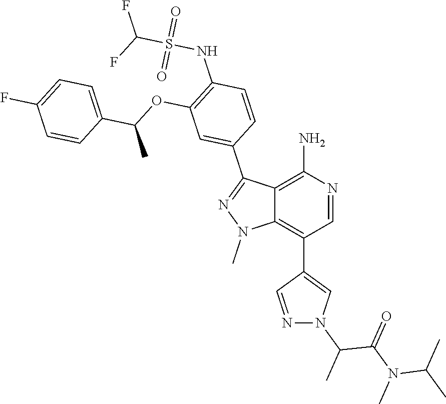 [SMILES: CC(C)N(C)C(=O)C(C)n1cc(-c2cnc(N)c3c(-c4ccc(NS(=O)(=O)C(F)F)c(O[C@@H](C)c5ccc(F)cc5)c4)nn(C)c23)cn1] | 36% | (Method A): 2.66 min, m/z: 685.3 [M + H]⁺ | (400 MHz, DMSO-d₆) 10.61 (s, 1H), 8.00-7.94 (m, 1H), 7.59-7.53 (m, 4H), 7.42-7.40 (m, 1H), 7.19-6.86 (m, 5H), 5.75- 5.74 (m, 2H), 5.70-5.63 (m, 1.5H), 5.58-5.53 (m, 0.5H), 4.67-4.60 | Intermediate C20 |
| 168 | N-(4-(4-amino-7-(1-(3- (dimethylamino)cyclopentyl)-1H- pyrazol-4-yl)-1-methyl-1H- pyrazolo[4,3-c]pyridin-3-yl)-2-((S)- 1-(4-fluorophenyl)ethoxy)phenyl)- 1,1-difluoromethanesulfonamide | 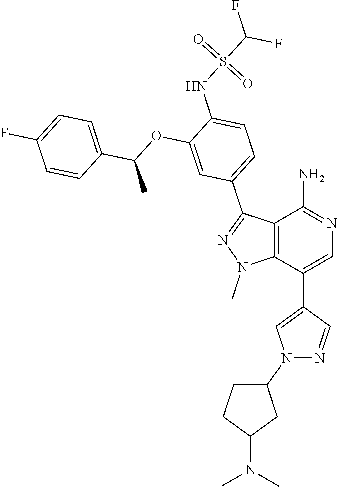 [SMILES: C[C@H](Oc1cc(-c2nn(C)c3c(-c4cnn(C5CCC(N(C)C)C5)c4)cnc(N)c23)ccc1NS(=O)(=O)C(F)F)c1ccc(F)cc1] | 11% | (Method A): 2.34 min, m/z: 669.2 [M + H]⁺ | (400 MHz, DMSO-d₆) 8.25 (s, 1H), 7.97 (s, 1H), 7.62 (s, 1H), 7.56-7.51 (m, 3H), 7.36 (d, J = 8 Hz, 1H), 7.14 (t, J = 8.4 Hz, 2H), 7.01 (dd, J = 2, 8 Hz, 1H), 6.96 (d, J = 2 Hz, 1H), 6.62 (t, J = 54 Hz, 1H), 5.75-5.62 (m, 3H), 4.88 (s, 1H), 3.67 (s, 3H), 3.57-3.49 (m, 1H), 2.58 (s, 6H), 2.32-1.74 (m, 7H), 1.54 (d, J = 6 Hz, 3H) | Intermediate C22 |

### General Procedure A2 for the Suzuki Reaction Followed by Acidic Deprotection Between Intermediate A1B1 and Boc Protected Boronate Esters

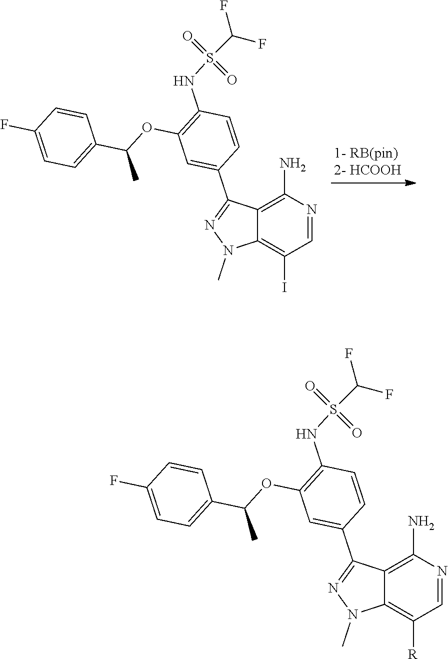

A mixture of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (1 eq), Boc protected boronate ester (1.1 eq), Na₂CO₃ (3 eq) and Pd(dppf)Cl₂ (0.1 eq) in degassed 1,4-dioxane/H₂O (4/1, 0.2 M) was stirred at 100° C. under N₂ overnight (or at 80° C. for 2 h under μW irradiation). The mixture was diluted with water and extracted with EtOAc (3×). The combined organics were dried over Na₂SO₄ and concentrated under reduced pressure. The residue was purified by column chromatography (DCM/MeOH) to give the Boc protected products.

The solids were dissolved in HCOOH neat (2 M) and the reaction mixture was stirred at RT for 30 min or until completion. The reaction mixture was concentrated under reduced pressure and the residue was adjusted to pH=8 with sat. aq. Na₂CO₃ solution. The residue was further diluted with water and the organics were extracted with EtOAc (3 times). The combined organics were washed with brine, dried over Na₂SO₄ and concentrated under reduced pressure. The residue was purified by prep-TLC (DCM/MeOH) followed by preparative HPLC (neutral condition method C) if needed to give the desired products

The following compounds were prepared following the General procedure A2.

TABLE 4
Compounds prepared following General procedure A2

| Com- | | | Yield | | | |
|---|---|---|---|---|---|---|
| pound | | | (over 2 | LCMS | | Boronate |
| No | Name | Structure | steps) | data | ¹H NMR data | ester |
| 59 | (S)-N-(4- (4-amino-1- methyl-7- (1-(piperidin-4- yl)-1H- pyrazol-4-yl)-1H- pyrazolo [4,3-c]pyridin-3- yl)-2-(1-(4- fluorophenyl) ethoxy) phenyl)-1,1- difluoromethane- sulfonamide | 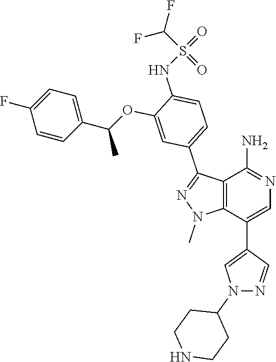 [SMILES: C[C@H](Oc1cc(-c2nn(C)c3c(-c4cnn(C5CCNCC5)c4)cnc(N)c23)ccc1NS(=O)(=O)C(F)F)c1ccc(F)cc1] | 17% | LCMS (Method A): 2.72 min, m/z: 641.2 [M + H]⁺. | (400 MHz, DMSO-d₆): 7.89 (s, 1H), 7.63 (s, 1H), 7.51- 7.48 (m, 3H), 7.35 (d, J = 8.4 Hz, 1H), 7.12 (t, J = 8.8 Hz, 2H), 6.91 (dd, J = 8.0, 0.8 Hz, 1H), 6.84 (s, 1H), 6.32 (t, J = 54.8 Hz, 1H), 5.64- 5.62 (m, 3H), 4.49-4.44 (m, 1H), 3.65 (s, 3H), 3.34-3.32 (m, 2H), 3.02-2.96 (m, 2H), 2.20-2.02 (m, 4H), 1.51 (d, J = 6.4 Hz, 3H). | tert-butyl 4-[4- (4,4,5,5- tetramethyl- 1,3,2- dioxa- borolan- 2-yl)- 1H-pyrazol- 1-yl] piperidine- 1- carboxylate |
| 95 | N-(4-(4-amino- 1-methyl- 7-(1-(5- methylpiperidin-3- yl)-1H-pyrazol- 4-yl)-1H- pyrazolo[4,3-c] pyridin-3- yl)-2-((S)-1-(4- fluorophenyl) ethoxy) phenyl)-1,1- difluoromethane- sulfonamide | 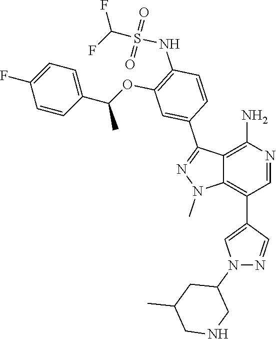 [SMILES: CC1CNCC(n2cc(-c3cnc(N)c4c(-c5ccc(NS(=O)(=O)C(F)F)c(O[C@@H](C)c6ccc(F)cc6)c5)nn(C)c34)cn2)C1] | 26% | (Method A): 2.67 min, m/z: 655.2 [M + H]⁺. | (400 MHz, DMSO-d₆): 9.26 (s, 1H), 8.15 (s, 1H), 7.74 (s, 1H), 7.56-7.53 (m, 3H), 7.39 (d, J = 8.0 Hz, 1H), 7.16 (t, J = 8.8 Hz, 2H), 7.07 (d, J = 8.0 Hz, 1H), 7.02 (s, 1H), 6.96 (s, 0.3H), 6.83 (s, 0.4H), 6.70 (s, 0.3H), 5.74 (s, 1H), 5.64 (q, J = 6.0 Hz, 1H), 4.78 (s, 1H), 3.77 (s, 2H), 3.72 (s, 3H), 3.17 (d, J = 12.0 Hz, 2H), 2.73 (t, J = 10.8 Hz, 1H), 2.24 (d, J = 14.0 Hz, 1H), 2.02- 1.97 (m, 1H), 1.84-1.78 (m, 1H), 1.56 (d, J = 6.0 Hz, 3H), 0.97 (d, J = 6.8 Hz, 3H). | Intermediate C12 |
| 88 | N-(4-(4- amino-1-methyl- 7-(1-(pyrrolidin- 2- ylmethyl)- 1H-pyrazol-4- yl)-1H- pyrazolo[4,3- c]pyridin-3- yl)-2-((S)-1-(4- fluorophenyl) ethoxy) phenyl)-1,1- difluoromethane- sulfonamide | 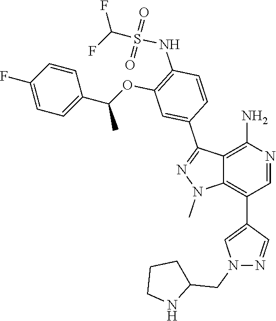 [SMILES: C[C@H](Oc1cc(-c2nn(C)c3c(-c4cnn(CC5CCCN5)c4)cnc(N)c23)ccc1NS(=O)(=O)C(F)F)c1ccc(F)cc1] | 17% | (Method A): 2.06 min, m/z: 641.2 [M + H]⁺. | (400 MHz, DMSO-d₆): 7.96 (s, 1H), 7.71 (s, 1H), 7.50 (d, J = 8.0 Hz, 3H), 7.36-7.34 (m, 1H), 7.12 (d, J = 12.0 Hz, 2H), 6.94-6.92 (m, 1H), 6.87- 6.86 (brs, 1H), 5.65-5.64 (m, 3H), 4.46-4.33 (m, 2 H), 3.88- 3.84 (m, 1H), 3.88 (s, 3H), 3.31-3.09 (m, 2H), 2.04-1.86 (m, 1H), 1.84-1.81 (m, 2H), 1.67-1.50 (m, 4 H). | Intermediate C13 |
| 94 | (S)-N-(4-(4- amino-7-(1- (azetidin-3- yl)-1H- pyrazol-4-yl)- 1-methyl- 1H-pyrazolo[4,3- c]pyridin-3-yl)- 2-(1-(4- fluorophenyl) ethoxy) phenyl)-1,1- difluoromethane- sulfonamide | 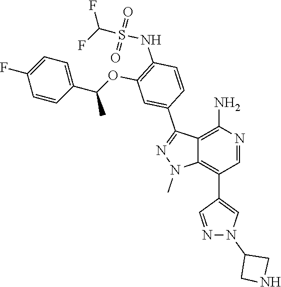 [SMILES: C[C@H](Oc1cc(-c2nn(C)c3c(-c4cnn(C5CNC5)c4)cnc(N)c23)ccc1NS(=O)(=O)C(F)F)c1ccc(F)cc1] | 13% | (Method A): 2.94 min, m/z: 613.0 [M + H]⁺. | (400 MHz, DMSO-d₆): 9.23 (s, 1H), 8.12 (s, 1H), 7.88 (s, 1H), 7.61-7.54 (m, 3H), 7.42 (d, J = 8.0 Hz, 1H), 7.22-6.89 (m, 5H), 6.36 (s, 1H), 5.64 (q, J = 6.2 Hz, 1H), 5.49 (p, J = 7.6 Hz, 1H), 4.48-4.37 (m, 4H), 3.73 (s, 3H), 1.58 (d, J = 6.3 Hz, 3H). | tert-butyl 3-[4- (4,4,5,5- tetramethyl- 1,3,2- dioxa- borolan- 2-yl)- 1H-pyrazol- 1- yl]azetidine- 1- carboxylate |
| 90 | (S)-N-(4-(4- amino-1- methyl-7- (4,5,6,7- tetrahydro- pyrazolo[1,5- a]pyrazin-3-yl)-1H- pyrazolo[4,3-c] pyridin-3- yl)-2-(1-(4- fluorophenyl)ethoxy) phenyl)-1,1- difluoromethane- sulfonamide | 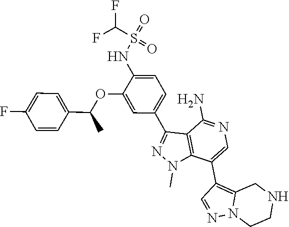 [SMILES: C[C@H](Oc1cc(-c2nn(C)c3c(-c4cnn5c4CNCC5)cnc(N)c23)ccc1NS(=O)(=O)C(F)F)c1ccc(F)cc1] | 14% | (Method A): 2.74 min, m/z: 613.2 [M + H]⁺. | (400 MHz, MeOD-d₄): 8.29 (s, 1H), 7.66 (s, 1H), 7.55 (d, J = 8.1 Hz, 1H), 7.51-7.46 (m, 3H), 7.18 (d, J = 8.1 Hz, 1H), 7.11-7.04 (m, 3H), 5.57 (q, J = 6.4 Hz, 1H), 4.31 (t, J = 5.7 Hz, 2H), 4.06 (s, 2H), 3.74 (s, 3H), 3.47 (t, J = 5.7 Hz, 2H), 1.70 (d, J = 6.3 Hz, 3H). | tert-butyl 3- bromo- 6,7- dihydro- pyrazolo [1,5-a] pyrazine- 5(4H)- carboxylate |
| 78 | (S)-N-(4-(7- (1-(2- azaspiro[3.3] heptan-6- yl)-1H-pyrazol- 4-yl)-4- amino-1- methyl-1H- pyrazolo[4,3-c] pyridin-3- yl)-2-(1-(4- fluorophenyl) ethoxy) phenyl)-1,1- difluoromethane- sulfonamide | 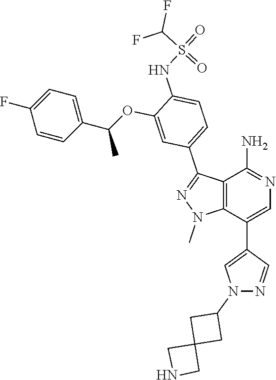 [SMILES: C[C@H](Oc1cc(-c2nn(C)c3c(-c4cnn(C5CC6(CNC6)C5)c4)cnc(N)c23)ccc1NS(=O)(=O)C(F)F)c1ccc(F)cc1] | 26% | (Method A): 2.75 min, m/z: 653.2 [M + H]⁺. | (400 MHz, DMSO-d₆): 8.00 (d, J = 2.4 Hz, 1H), 7.64 (d, J = 1.8 Hz, 1H), 7.58-7.52 (m, 3H), 7.40 (d, J = 8.0 Hz, 1H), 7.20-7.06 (m, 5H), 5.68 (d, J = 9.5 Hz, 2H), 5.65 (d, J = 6.3 Hz, 1H), 4.83 (p, J = 8.0 Hz, 1H), 4.23 (s, 1H), 4.14 (s, 1H), 3.95 (s, 1H), 3.86 (s, 1H), 3.70 (s, 3H), 2.74-2.65 (m, 4H), 1.74 (d, J = 5.2 Hz, 3H). | Intermediate C14 |
| 74 | N-(4-(4-amino- 7-(1- (azepan-4-yl)-1H- pyrazol-4-yl)- 1-methyl- 1H-pyrazolo[4,3- c]pyridin-3-yl)- 2-((S)-1-(4- fluorophenyl) ethoxy) phenyl)-1,1- difluoromethane- sulfonamide | 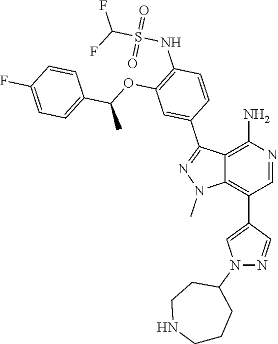 [SMILES: C[C@H](Oc1cc(-c2nn(C)c3c(-c4cnn(C5CCCNCC5)c4)cnc(N)c23)ccc1NS(=O)(=O)C(F)F)c1ccc(F)cc1] | 14% | (Method C): 3.15 min, m/z: 655.2 [M + H]⁺. | (400 MHz, DMSO-d₆) 8.52 (s, 1H), 7.94-7.88 (m, 1H), 7.53-7.48 (m, 4H), 7.34-7.29 (m, 1H), 7.11 (t, J = 8.0 Hz, 2H), 6.89-6.81 (m, 2H), 6.38- 6.10 (m, 1H), 5.67-5.60 (m, 3H), 4.48-4.42 (m, 1H), 3.65 (s, 3H), 2.91-2.70 (m, 3H), 2.11-1.97 (m, 6H), 1.76-1.72 (m, 1H), 1.63-1.57 (m, 1H), 1.49 (d, J = 6.4 Hz, 3H). | Intermediate C15 |

The following compounds were prepared following the General procedure A1 with 1-(tetrahydro-2H-pyran-4-yl)-4-(4,4,5,5-tetramethyl-1,3,2-dioxaborolan-2-yl)-1H-pyrazole and intermediate B32-B35

TABLE 5
Compounds prepared following the General procedure A1 with 1-(tetrahydro-2H-pyran-4-yl)-4-(4,4,5,5-tetramethyl-1,3,2-
dioxaborolan-2-yl)-1H-pyrazole and intermediates B2-B16

| Com- | | | | | Inter- |
|---|---|---|---|---|---|
| pound | | | LCMS | | med- |
| No | Name | Structure | data | 1H NMR data | iate B |
| 121 | (R)-N-(4- (4-amino- 1-methyl-7-(1- (tetrahydro-2H- pyran-4-yl)-1H- pyrazol-4-yl)-1H- pyrazolo[4,3-c] pyridin-3-yl)-2- (1-(4- fluorophenyl) ethoxy)phenyl)- 1,1- difluoromethane- sulfonamide | 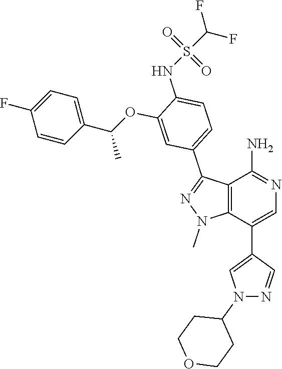 [SMILES: C[C@@H](Oc1cc(-c2nn(C)c3c(-c4cnn(C5CCOCC5)c4)cnc(N)c23)ccc1NS(=O)(=O)C(F)F)c1ccc(F)cc1] | (Method C): 3.10 min, m/z: 642.0 [M + H]⁺. | (400 MHz, DMSO-d₆) 10.64 (s, 1H), 8.02 (s, 1H), 7.61 (s, 1H), 7.58-7.53 (m, 3H), 7.40 (d, J = 8.0 Hz, 1H), 7.20-7.09 (m, 4H), 6.93 (d, J = 52.7 Hz, 1H), 5.74 (s, 2H), 5.65 (q, J = 6.2 Hz, 1H), 4.45 (tt, J = 10.3, 5.3 Hz, 1H), 4.00-3.93 (m, 2H), 3.69 (s, 3H), 3.48 (td, J = 11.4, 3.5 Hz, 2H), 2.00 (td, J = 11.2, 10.3, 4.5 Hz, 4H), 1.57 (d, J = 6.3 Hz, 3H). | B2 |
| 122 | (S)-N-(4- (4-amino- 1-methyl-7-(1- (tetrahydro-2H- pyran-4-yl)-1H- pyrazolo[4,3-c] pyridin-3-yl)-2- (1-(3- chlorophenyl) ethoxy)phenyl)- 1,1- difluoromethane- sulfonamide | 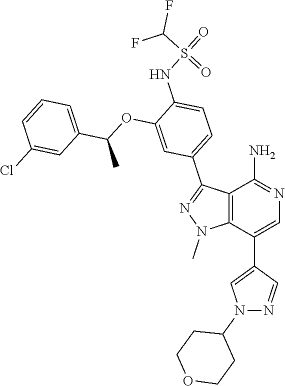 [SMILES: C[C@H](Oc1cc(-c2nn(C)c3c(-c4cnn(C5CCOCC5)c4)cnc(N)c23)ccc1NS(=O)(=O)C(F)F)c1cccc(Cl)c1] | (Method A): 3.07 min, m/z: 658.0 [M + H]⁺. | (400 MHz, DMSO-d₆) 8.03 (s, 1H), 7.67 (s, 1H), 7.62 (s, 1H), 7.55 (s, 1H), 7.48 (d, J = 8.0 Hz, 1H), 7.43-7.31 (m, 3H), 7.15-7.09 (m, 2H), 7.00 (t, J = 52 Hz, 1H), 5.71-5.66 (m, 2H), 4.47-4.44 (m, 1H), 4.04-3.96 (m, 2H), 3.70 (s, 3H), 3.50-3.38 (m, 2H), 2.03- 1.99 (m, 4H), 1.58 (d, J = 6.4 Hz, 3H). | B3 |
| 118 | (S)-N-(4- (4-amino- 1-methyl-7-(1- (tetrahydro-2H- pyran-4-yl)-1H- pyrazol-4-yl)-1H- pyrazolo[4,3-c] pyridin-3-yl)-2- (1-(2-fluoro- phenyl)ethoxy) phenyl)-1,1- difluoromethane- sulfonamide | 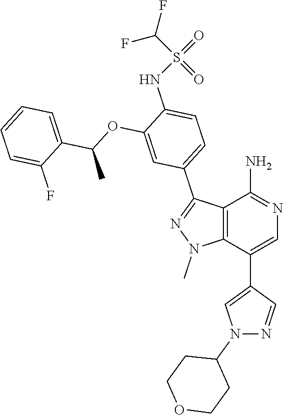 [SMILES: C[C@H](Oc1cc(-c2nn(C)c3c(-c4cnn(C5CCOCC5)c4)cnc(N)c23)ccc1NS(=O)(=O)C(F)F)c1ccccc1F] | (Method A): 2.98 min, m/z: 642.0 [M + H]⁺. | (400 MHz, DMSO-d₆) 8.06 (s, 1H), 7.67 (d, J = 8.0 Hz, 1H), 7.63 (s, 1H), 7.57 (s, 1H), 7.42 (d, J = 8.0 Hz, 1H), 7.35-7.30 (m, 1H), 7.22-7.14 (m, 3H), 7.02 (s, 1H), 6.88 (s, 0.2H), 6.23 (s, 1.5H), 5.81 (q, J = 6.0 Hz, 1H), 4.50-4.42 (m, 1H), 3.97 (d, J = 11.2 Hz, 2H), 3.69 (s, 3H), 3.50 (d, J = 11.2 Hz, 2H), 2.02-1.99 (m, 4H), 1.63 (d, J = 6.4 Hz, 3H). | B4 |
| 102 | N-(4-(4-amino- 1-methyl-7-(1- (tetrahydro-2H- pyran-4-yl)-1H- pyrazol-4-yl)-1H- pyrazolo[4,3-c] pyridin-3-yl)-2- (1-(4- fluorophenyl)- 2-methoxyethoxy) phenyl)-1,1- difuoromethane- sulfonamide | 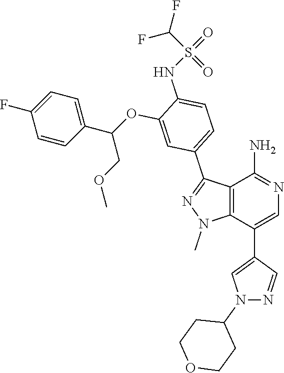 [SMILES: COCC(Oc1cc(-c2nn(C)c3c(-c4cnn(C5CCOCC5)c4)cnc(N)c23)ccc1NS(=O)(=O)C(F)F)c1ccc(F)cc1] | (Method A): 3.11 min, m/z: 672.1 [M + H]⁺. | (400 MHz, CDCl₃) 7.75 (d, J = 8.4 Hz, 1H), 7.61 (s, 1H), 7.55 (s, 1H), 7.50 (s, 1H), 7.44-7.37 (m, 2H), 7.33 (dd, J = 8.4, 1.9 Hz, 1H), 7.08 (t, J = 8.6 Hz, 2H), 6.97 (d, J = 1.9 Hz, 1H), 6.38 (t, J = 53.6 Hz, 1H), 5.00 (dd, J = 9.4, 2.7 Hz, 1H), 4.42 (tt, J = 10.7, 4.8 Hz, 1H), 4.19-4.09 (m, 2H), 3.81 (t, J = 9.9 Hz, 1H), 3.73 (s, 3H), 3.65 (t, J = 6.6 Hz, 1H), 3.61-3.55 (m, 2H), 3.54 (s, 3H), 2.15 (qd, J = 12.6, 11.8, 4.3 Hz, 4H). | B5 |
| 132 | N-(4-(4-amino- 1-methyl-7-(1- (tetrahydro-2H- pyran-4-yl)-1H- pyrazol-4-yl)-1H- pyrazolo[4,3-c] pyridin-3-yl)- 2-((4- chloro-3-fluoro- benzyl)oxy) phenyl)-1,1- difluoromethane- sulfonamide | 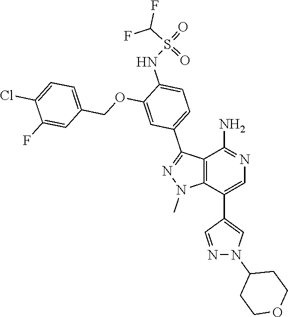 [SMILES: Cn1nc(-c2ccc(NS(=O)(=O)C(F)F)c(OCc3ccc(Cl)c(F)c3)c2)c2c(N)ncc(-c3cnn(C4CCOCC4)c3)c21] | (Method A): 3.08 min, m/z: 662.0 [M + H]+. | (400 MHz, DMSO-d₆) 8.04 (s, 1H), 7.73- 7.66 (m, 1H), 7.64 (d, J = 7.7 Hz, 2H), 7.60 (d, J = 12.1 Hz, 1H), 7.43 (dd, J = 14.4, 8.3 Hz, 2H), 7.34 (d, J = 1.8 Hz, 1H), 7.23 (dd, J = 8.0, 1.8 Hz, 1H), 7.08-6.76 (m, 1H), 5.89 (s, 2H), 5.26 (s, 2H), 4.47 (tt, J = 10.3, 5.5 Hz, 1H), 4.04-3.94 (m, 2H), 3.74 (s, 3H), 3.50 (dt, J = 10.8, 5.4 Hz, 3H), 2.03 (q, J = 4.4 Hz, 3H). | B6 |
| 129 | (S)-N-(4- (4-amino- 1-methyl-7-(1- (tetrahydro-2H- pyran-4-yl)-1H- pyrazol-4-yl)-1H- pyrazolo[4,3-c] pyridin-3-yl)- 2-(1- (4-methoxy- phenyl)ethoxy) phenyl)-1,1- difluoromethane- sulfonamide | 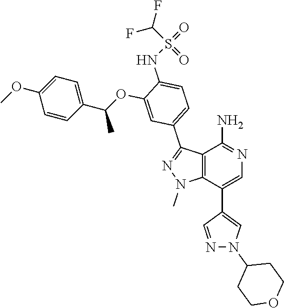 [SMILES: COc1ccc([C@H](C)Oc2cc(-c3nn(C)c4c(-c5cnn(C6CCOCC6)c5)cnc(N)c34)ccc2NS(=O)(=O)C(F)F)cc1] | (Method A): 1.70 min, m/z: 652.2 [M + H]⁺. | (400 MHz, DMSO-d₆) 8.04 (d, J = 8.1 Hz, 1H), 7.64 (d, J = 6.9 Hz, 1H), 7.57 (s, 1H), 7.47-7.23 (m, 3H), 7.15-7.07 (m, 2H), 6.99 (d, J = 6.8 Hz, 1H), 6.93-6.86 (m, 2H), 6.08-5.51 (m, 3H), 4.49 (s, 1H), 4.00 (d, J = 10.9 Hz, 3H), 3.74 (s, 3H), 3.71 (s, 3H), 2.03 (d, J = 18.1 Hz, 5H), 1.60 (d, J = 6.2 Hz, 1H), 1.38-1.17 (m, 3H). | B7 |
| 130 | N-(4-(4-amino- 1-methyl-7-(1- (tetrahydro-2H- pyran-4-yl)-1H- pyrazol-4-yl)-1H- pyrazolo[4,3-c] pyridin-3-yl)- 2-((3- chlorobenzyl) oxy)phenyl)-1,1- difluoromethane- sulfonamide | 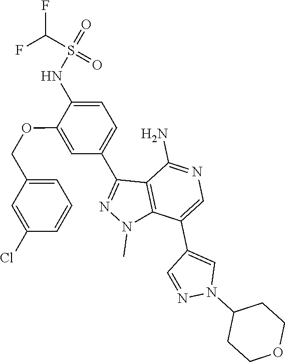 [SMILES: Cn1nc(-c2ccc(NS(=O)(=O)C(F)F)c(OCc3cccc(Cl)c3)c2)c2c(N)ncc(-c3cnn(C4CCOCC4)c3)c21] | (Method A): 3.17 min, m/z: 644.1 [M + H]⁺. | (400 MHz, DMSO-d₆) 8.03 (s, 1H), 7.68 (s, 1H), 7.63 (s, 1H), 7.58 (s, 1H), 7.50 (d, J = 7.2 Hz, 1H), 7.46-7.32 (m, 4H), 7.23 (dd, J = 8.1, 1.8 Hz, 1H), 6.93 (t, J = 52.8 Hz, 1H), 5.86 (s, 2H), 5.25 (s, 2H), 4.47 (tt, J = 10.5, 5.5 Hz, 1H), 3.98 (dt, J = 12.2, 3.0 Hz, 2H), 3.74 (s, 3H), 3.51 (dd, J = 11.2, 3.3 Hz, 2H), 2.03 (q, J = 4.4 Hz, 4H). | B8 |
| 136 | N-(4-(4-amino- 1-methyl-7-(1- (tetrahydro-2H- pyran-4-yl)-1H- pyrazol-4-yl)-1H- pyrazolo[4,3-c] pyridin-3-yl)- 2-((5- chloropyridin-2- yl)methoxy) phenyl)-1,1- difluoromethane- sulfonamide | 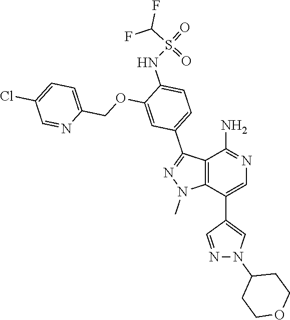 [SMILES: Cn1nc(-c2ccc(NS(=O)(=O)C(F)F)c(OCc3ccc(Cl)cn3)c2)c2c(N)ncc(-c3cnn(C4CCOCC4)c3)c21] | (Method A): 2.90 min, m/z: 645.1 [M + H]⁺. | (400 MHz, DMSO-d₆) 8.64 (d, J = 2.4 Hz, 1H), 8.05 (s, 2H), 7.80 (d, J = 8.4 Hz, 1H), 7.62 (d, J = 22.2 Hz, 2H), 7.45 (d, J = 8.0 Hz, 1H), 7.33 (d, J = 8.7 Hz, 1H), 7.22 (d, J = 7.8 Hz, 1H), 6.89 (t, J = 53.1 Hz, 1H), 5.88 (s, 2H), 5.32 (s, 2H), 4.55-4.44 (m, 1H), 3.99 (d, J = 5.7 Hz, 2H), 3.75 (s, 3H), 3.54 (d, J = 3.4 Hz, 2H), 2.05 (d, J = 5.1 Hz, 4H). | B9 |
| 140 | N-(4-(4-amino- 1-methyl-7-(1- (tetrahydro-2H- pyran-4-yl)-1H- pyrazol-4-yl)-1H- pyrazolo[4,3-c] pyridin-3-yl)- 2-(1- (4-fluorophenyl) cyclopropoxy) phenyl)-1,1- difluoromethane- sulfonamide | 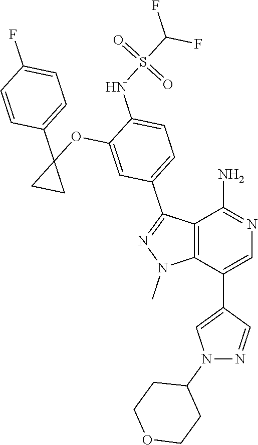 [SMILES: Cn1nc(-c2ccc(NS(=O)(=O)C(F)F)c(OC3(c4ccc(F)cc4)CC3)c2)c2c(N)ncc(-c3cnn(C4CCOCC4)c3)c21] | (Method A): 3.10 min, m/z: 654.1 [M + H]⁺. | (400 MHz, CDCl₃) 7.65 (d, J = 8.2 Hz, 1H), 7.60 (s, 1H), 7.54 (s, 1H), 7.46 (s, 1H), 7.24 (d, J = 1.9 Hz, 1H), 7.18 (d, J = 1.8 Hz, 1H), 7.12 (dd, J = 8.7, 5.1 Hz, 2H), 6.98 (t, J = 8.6 Hz, 2H), 6.35 (t, J = 53.8 Hz, 1H), 4.95 (s, 2H), 4.40 (ddd, J = 15.8, 10.7, 4.5 Hz, 1H), 4.18-4.09 (m, 2H), 3.73 (s, 3H), 3.57 (td, J = 11.6, 2.8 Hz, 2H), 2.20-2.02 (m, 5H), 1.43 (d, J = 5.4 Hz, 2H), 1.34 (d, J = 6.1 Hz, 3H). | B10 |
| 143 | N-(4-(4-amino- 1-methyl-7-(1- (tetrahydro-2H- pyran-4-yl)-1H- pyrazol-4-yl)-1H- pyrazolo[4,3-c] pyridin-3-yl)-2- (thiazol-2-yl- methoxy)phenyl)- 1,1- difluoromethane- sulfonamide | 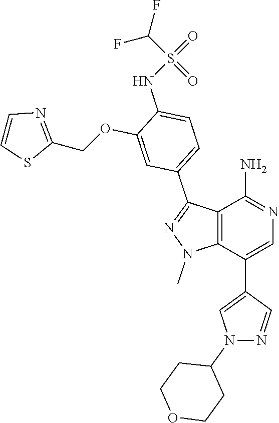 [SMILES: Cn1nc(-c2ccc(NS(=O)(=O)C(F)F)c(OCc3nccs3)c2)c2c(N)ncc(-c3cnn(C4CCOCC4)c3)c21] | (Method A): 2.77 min, m/z: 617.1 [M + H]⁺. | (400 MHz, CDCl₃) 7.83 (d, J = 3.2 Hz, 1H), 7.72 (d, J = 8.2 Hz, 1H), 7.62 (s, 1H), 7.59 (s, 1H), 7.55 (s, 1H), 7.43 (d, J = 3.2 Hz, 1H), 7.39 (d, J = 1.8 Hz, 1H), 7.32 (d, J = 8.2 Hz, 1H), 6.33 (t, J = 53.6 Hz, 1H), 5.80 (s, 1H), 5.53 (s, 2H), 4.44 (dt, J = 11.0, 5.8 Hz, 1H), 4.15 (d, J = 11.9 Hz, 2H), 3.79 (s, 3H), 3.65-3.53 (m, 2H), 2.22-2.10 (m, 4H) | B11 |
| 144 | N-(4-(4-amino- 1-methyl-7-(1- (tetrahydro-2H- pyran-4-yl)-1H- pyrazol-4-yl)-1H- pyrazolo[4,3-c] pyridin-3-yl)-2- isopropoxy- phenyl)- 1,1- difluoromethane- sulfonamide | 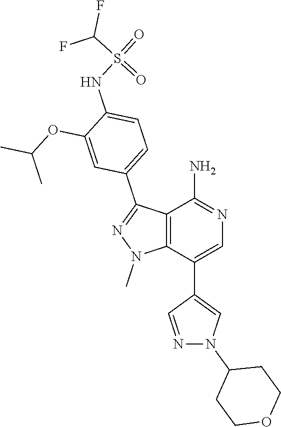 [SMILES: CC(C)Oc1cc(-c2nn(C)c3c(-c4cnn(C5CCOCC5)c4)cnc(N)c23)ccc1NS(=O)(=O)C(F)F] | (Method A): 4.25 min, m/z: 562.2 [M + H]⁺. | (400 MHz, DMSO-d₆) 8.03 (s, 1H), 7.62 (s, 1H), 7.58 (s, 1H), 7.39 (dd, J = 8.1, 3.0 Hz, 1H), 7.26-7.22 (m, 1H), 7.15 (dd, J = 8.0, 1.8 Hz, 1H), 5.96 (s, 2H), 4.73 (p, J = 6.0 Hz, 1H), 4.47 (td, J = 10.2, 5.0 Hz, 1H), 4.01-3.96 (m, 2H), 3.74 (s, 3H), 3.49 (d, J = 3.2 Hz, 2H), 3.17 (s, 1H), 2.02 (td, J = 10.8, 10.0, 4.0 Hz, 4H), 1.33 (d, J = 5.9 Hz, 6H). | B12 |
| 150 | N-(4-(4-amino- 1-methyl-7-(1- (tetrahydro-2H- pyran-4-yl)-1H- pyrazol-4-yl)-1H- pyrazolo[4,3-c] pyridin-3-yl)-2- (cyclohexyl- methoxy)phenyl)- 1,1- difluoromethane- sulfonamide | 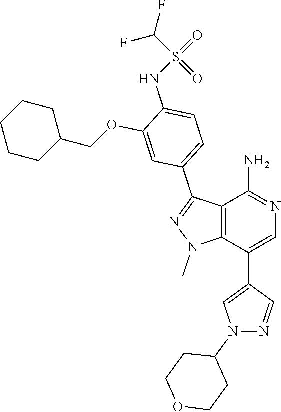 [SMILES: Cn1nc(-c2ccc(NS(=O)(=O)C(F)F)c(OCC3CCCCC3)c2)c2c(N)ncc(-c3cnn(C4CCOCC4)c3)c21] | (Method A): 2.40 min, m/z: 616.1 [M + H]⁺. | (400 MHz, MeOD-d₄) 7.91 (d, J = 3.7 Hz, 1H), 7.64 (s, 1H), 7.61-7.45 (m, 4H), 7.28 (d, J = 1.8 Hz, 1H), 7.20 (dd, J = 8.1, 1.8 Hz, 1H), 6.62 (t, J = 53.3 Hz, 1H), 4.50 (dd, J = 10.4, 5.0 Hz, 1H), 4.09 (dt, J = 11.5, 3.3 Hz, 2H), 3.92 (d, J = 6.2 Hz, 2H), 3.77 (s, 3H), 3.60 (td, J = 11.3, 3.8 Hz, 2H), 3.35 (s, 1H), 2.12 (q, J = 4.0 Hz, 3H), 1.95 (d, J = 12.8 Hz, 2H), 1.78 (d, J = 14.9 Hz, 2H), 1.42-1.22 (m, 6H), 1.12 (td, J = 12.1, 11.5, 4.4 Hz, 3H). | B13 |
| 151 | N-(4-(4-amino- 1-methyl-7-(1- (tetrahydro-2H- pyran-4-yl)-1H- pyrazol-4-yl)-1H- pyrazolo[4,3-c] pyridin-3-yl)-2- (oxazol-2-yl- methoxy)phenyl)- 1,1- difluoromethane- sulfonamide | 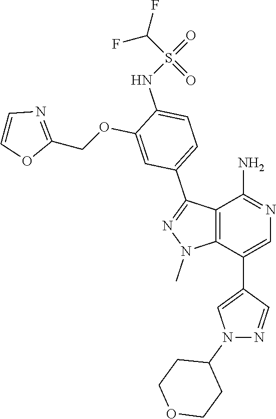 [SMILES: Cn1nc(-c2ccc(NS(=O)(=O)C(F)F)c(OCc3ncco3)c2)c2c(N)ncc(-c3cnn(C4CCOCC4)c3)c21] | (Method A): 2.34 min, m/z: 601.2 [M + H]⁺. | (400 MHz, DMSO-d₆) 10.98 (s, 1H), 8.21 (s, 1H), 8.06 (s, 1H), 7.65 (s, 1H), 7.60 (s, 1H), 7.48 (s, 1H), 7.42 (d, J = 12 Hz, 1H), 7.30 (s, 1H), 7.25 (d, J = 8 Hz, 1H), 6.96 (t, J = 52 Hz, 1H), 6.20 (s, 2H), 5.37 (s, 2H), 4.51-5.43 (m, 1H), 4.00-3.97 (m, 2H), 3.75 (s, 3H), 3.53-3.46 (m, 2H), 2.05-1.99 (m, 4H). | B14 |
| 137 | (S)-N-(4-(4- amino-1-methyl-7- (1-(tetrahydro-2H- pyran-4-yl)-1H- pyrazolo[4,3-c] pyridin-3-yl)-2-(1- (4-(chlorophenyl) ethoxy)phenyl)- 1,1- difluoromethane- sulfonamide | 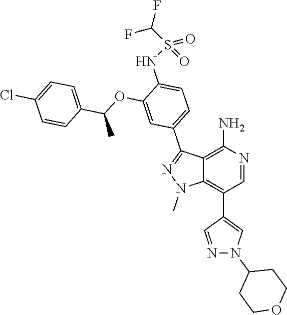 [SMILES: C[C@H](Oc1cc(-c2nn(C)c3c(-c4cnn(C5CCOCC5)c4)cnc(N)c23)ccc1NS(=O)(=O)C(F)F)c1ccc(Cl)cc1] | (Method A): 3.08 min, m/z: 658.1 [M + H]⁺. | (400 MHz, DMSO-d₆) 10.66 (s, 1H), 8.02 (s, 1H), 7.61 (s, 1H), 7.55 (d, J = 7.4 Hz, 3H), 7.41 (d, J = 7.9 Hz, 3H), 7.14 (d, J = 7.5 Hz, 1H), 7.09 (s, 1H), 7.00 (s, 1H), 5.66 (d, J = 6.6 Hz, 1H), 4.46 (dt, J = 10.2, 5.6 Hz, 1H), 3.98 (d, J = 11.8 Hz, 2H), 3.69 (s, 3H), 3.49 (ddd, J = 14.0, 10.8, 3.3 Hz, 2H), 2.06-1.97 (m, 4H), 1.58 (d, J = 6.2 Hz, 3H). | B15 |
| 169 | N-(4-(4-amino- 1-methyl-7-(1- (tetrahydro-2H- pyran-4-yl)-1H- pyrazol-4-yl)-1H- pyrazolo[4,3-c] pyridin-3-yl)-2- (benzo[d][1,3] dioxol-5- ylmethoxy) phenyl)-1,1- difluoromethane- sulfonamide | 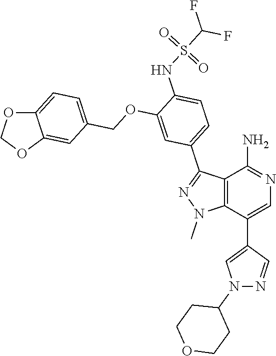 [SMILES: Cn1nc(-c2ccc(NS(=O)(=O)C(F)F)c(OCc3ccc4c(c3)OCO4)c2)c2c(N)ncc(-c3cnn(C4CCOCC4)c3)c21] | (Method A): 2.91 min, m/z: 654.1 [M + H]⁺. | (400 MHz, DMSO-d₆) 8.04 (s, 1H), 7.63 (s, 1H), 7.58 (s, 1H), 7.41 (d, J = 8 Hz, 1H), 7.34 (s, 1H), 7.21 (d, J = 8 Hz, 1H), 7.17 (s, 1H), 7.01-6.90 (m, 2H), 6.77 (t, J = 52 Hz, 1H), 6.02 (s, 2H), 5.87 (s, 1H), 5.13 (s, 2H), 3.98 (d, J = 12 Hz, 2H), 3.67 (s, 3H), 3.46-3.42 (m, 2H), 2.05-1.99 (m, 4H). | B16 |

### Synthesis of Boronate Ester Intermediates C

### Intermediate C1: 4-(4-(4,4,5,5-tetramethyl-1,3,2-dioxaborolan-2-yl)-1H-pyrazol-1-yl)-1-(2,2,2-trifluoroethyl)piperidine

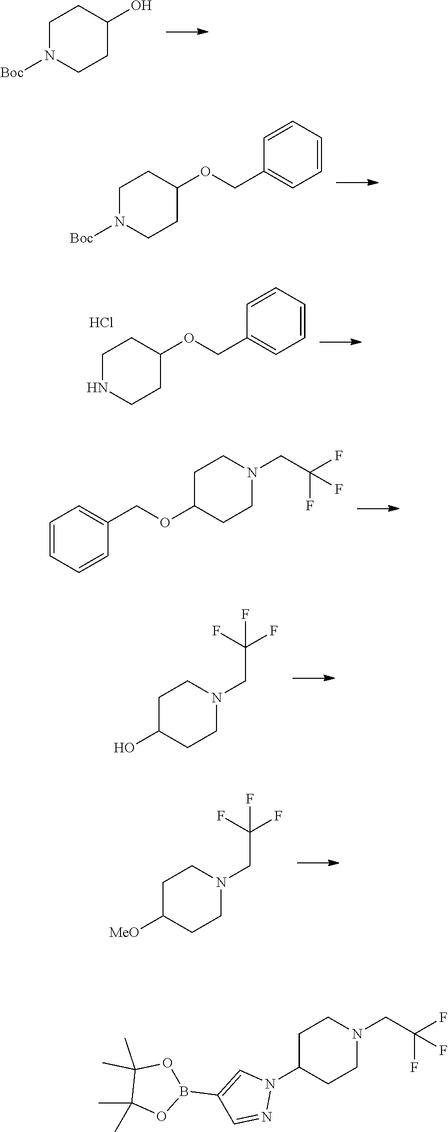

### Step 1: tert-butyl 4-(benzyloxy)piperidine-1-carboxylate

To a solution of tert-butyl 4-hydroxypiperidine-1-carboxylate (1 g, 4.96 mmol) in dry DMF (10 mL) at 0° C. was added NaH (60% in oil, 238 mg, 9.92 mmol). The mixture was stirred at 0° C. for 30 mins, then (chloromethyl)benzene (755 mg, 5.95 mmol) was added and the mixture was stirred at RT overnight. The reaction mixture was partitioned between water (10 mL) and EtOAc (10 mL). The aqueous layer was extracted with EtOAc (3×30 mL) and the combined organics were washed with brine, dried over Na₂SO₄ and concentrated under reduced pressure. The residue was purified by column chromatography (PE/EtOAc=10/1) to give the title product (1.30 g, 92%) as a colourless oil. ¹HNMR (400 MHz, MeOD-d₄): 7.27-7.18 (m, 5H), 4.48 (s, 2H), 3.73-3.67 (m, 2H), 3.51-3.46 (m, 1H), 3.06-3.00 (m, 2H), 1.80-1.75 (m, 2H), 1.55-1.48 (m, 2H), 1.38 (s, 9H).

### Step 2: 4-(benzyloxy)piperidine hydrochloride

To a solution of tert-butyl 4-(benzyloxy)piperidine-1-carboxylate (1.3 g, 4.46 mmol) in dioxane (5 mL) was added HCl in dioxane (4 M, 10 mL) and the reaction mixture was stirred at RT overnight. The solution was concentrated under reduced pressure to give the title product (2.10 g, 100%) as a white solid. ¹HNMR (400 MHz, MeOD-d₄): 7.38-7.26 (m, 5H), 4.58 (s, 2H), 3.81-3.76 (m, 1H), 3.37-3.31 (m, 2H), 3.16-3.10 (m, 2H), 2.08-2.01 (m, 2H), 1.97-1.91 (m, 2H).

### Step 3: 4-(benzyloxy)-1-(2,2,2-trifluoroethyl)piperidine

To a solution of 4-(benzyloxy)piperidine hydrochloride (2.1 g, 9.22 mmol) in DMF (15 mL) was added 1,1,1-trifluoro-2-iodoethane (5.79 g, 27.6 mmol) and K₂CO₃ (3.81 g, 27.6 mmol). The reaction mixture was stirred at 130° C. for 1.5 h with μW irradiation. The reaction mixture was partitioned between water (10 mL) and EtOAc (10 ml), and the aqueous layer was extracted with EtOAc (2×20 mL). The combined organics were washed with brine, dried over Na₂SO₄ and concentrated under reduced pressure. The residue was purified by column chromatography (PE/EtOAc=10/1) to give the title product (896 mg, 36%) as a yellow oil. ¹HNMR (400 MHz, MeOD-d₄): 7.36-7.24 (m, 5H), 4.54 (s, 2H), 3.50-3.43 (m, 1H), 3.07-2.99 (m, 2H), 2.92-2.87 (m, 2H), 2.51-2.45 (m, 2H), 1.95-1.89 (m, 2H), 1.70-1.61 (m, 2H).

### Step 4: 1-(2,2,2-trifluoroethyl)piperidin-4-ol

To a solution of 4-(benzyloxy)-1-(2,2,2-trifluoroethyl)piperidine (890 mg, 3.25 mmol) in MeOH (10 mL) was added Pd(OH)₂ (91.2 mg). The mixture was stirred at 55° C. under H₂ overnight. The reaction mixture was filtered, and the organics were concentrated under reduced pressure to give the title product (590 mg, 98%) as colorless oil. ¹HNMR (400 MHz, CDCl₃): 3.72-3.67 (m, 1H), 3.00-2.91 (m, 2H), 2.90-2.85 (m, 2H), 2.51-2.44 (m, 2H), 1.91-1.84 (m, 2H), 1.64-1.55 (m, 2H).

### Step 5: 4-(4-(4,4,5,5-tetramethyl-1,3,2-dioxaborolan-2-yl)-1H-pyrazol-1-yl)-1-(2,2,2-trifluoroethyl)piperidine

To a solution of 1-(2,2,2-trifluoroethyl)piperidin-4-ol (590 mg, 3.22 mmol) in DCM (10 mL) was added Et₃N (1.45 g, 14.4 mmol), followed by MsCl (1.10 g, 9.66 mmol), and the mixture was stirred at RT overnight. The mixture was partitioned between water (10 mL) and DCM (10 mL) and the organic layer was washed with brine, dried over Na₂SO₄ and concentrated under reduced pressure to give the crude mesylate (880 mg, 100%) as a yellow oil. ¹HNMR (400 MHz, CDCl₃): 4.78-4.73 (m, 1H), 3.13 (s, 1H), 3.01-2.94 (m, 7H), 2.90-2.85 (m, 2H), 2.64-2.60 (m, 2H), 2.05-1.99 (m, 2H), 1.94-1.88 (m, 2H).

The crude mesylate was dissolved in DMF (10 mL), and 4-(4,4,5,5-tetramethyl-1,3,2-dioxaborolan-2-yl)-1H-pyrazole (465 mg, 2.40 mmol), Cs₂CO₃ (1.30 g, 4.00 mmol) and KI (44.3 mg, 267 μmol) were added. The reaction mixture was stirred at 100° C. in a sealed tube overnight. Water (10 mL) was added, and the mixture was extracted with EtOAc (2×40 mL). The combined organics were washed with water and brine, dried over Na₂SO₄ and concentrated under reduced pressure. The residue was purified by prep-TLC (PE/EtOAc=70/30, v/v) to give the title product (70.0 mg, 7%) as a colourless oil. LCMS (method A): 1.36 min, m/z: 360.2 [M+H]⁺.

### Intermediate C2: 1-methyl-4-(2-(4-(4,4,5,5-tetramethyl-1,3,2-dioxaborolan-2-yl)-1H-pyrazol-1-yl)ethyl)piperazine

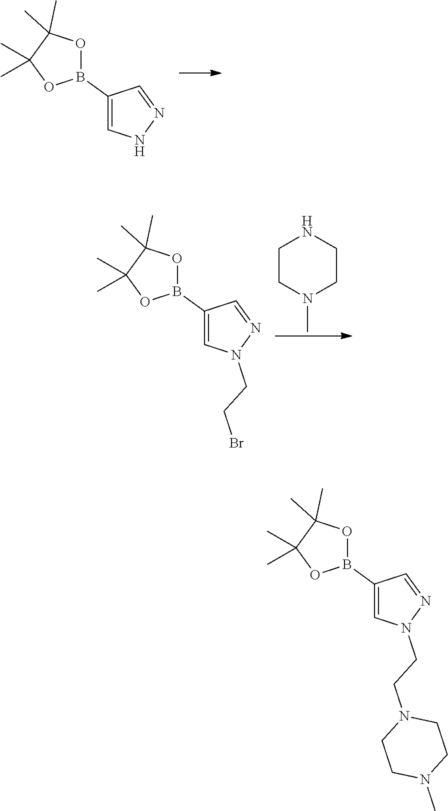

### Step 1: 1-(2-bromoethyl)-4-(4,4,5,5-tetramethyl-1,3,2-dioxaborolan-2-yl)-1H-pyrazole

To a solution of 4-(4,4,5,5-tetramethyl-1,3,2-dioxaborolan-2-yl)-1H-pyrazole (500 mg, 2.57 mmol) in DMSO (5 mL) was added KOH (1.43 g, 25.6 mmol). After stirring at RT for 1 h, 1,2-dibromoethane (9.63 g, 51.3 mmol) was added. The mixture was stirred at RT overnight, then diluted with water (10 mL) and extracted with EtOAc (3×15 mL). The combined organics were washed with brine, dried over Na₂SO₄ and concentrated under reduced pressure. The residue was purified by column chromatography (PE/EtOAc=5/1) to give the title product (320 mg, 41%) as a colourless oil. LCMS (Method A): 2.03 min, m/z 301.1/303.1 [M+H]⁺. ¹H NMR (400 MHz, DMSO-d₆): 7.99 (s, 1H), 7.62 (s, 1H), 4.52 (d, J=6.4 Hz, 2H), 3.85 (d, J=6.0 Hz, 2H), 1.26 (s, 12H).

### Step 2: 1-methyl-4-(2-(4-(4,4,5,5-tetramethyl-1,3,2-dioxaborolan-2-yl)-1H-pyrazol-1-yl)ethyl)piperazine

To a solution of 1-(2-bromoethyl)-4-(4,4,5,5-tetramethyl-1,3,2-dioxaborolan-2-yl)-1H-pyrazole (100 mg, 332 μmol) in MeCN (3 mL) were added 1-methylpiperazine (49.8 mg, 498 μmol) and Cs₂CO₃ (216 mg, 664 μmol). The mixture was stirred at 90° C. for 6 h. Water (10 mL) was added, and the organics were extracted with EtOAc (3×15 mL). The combined organics were washed with brine, dried over Na₂SO₄ and concentrated under reduced pressure to give the title product (60.0 mg, 56%) as a light-yellow oil, which was used directly without further purification. LCMS (Method B): 1.43 min, m/z: 321.3 [M+H]⁺.

### Intermediate C3: 1-(2-(3,3-dimethylazetidin-1-yl)ethyl)-4-(4,4,5,5-tetramethyl-1,3,2-dioxaborolan-2-yl)-1H-pyrazole

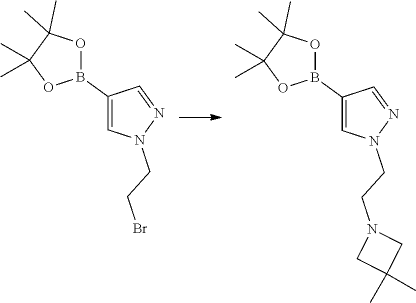

### Step 1: 1-(2-(3,3-dimethylazetidin-1-yl)ethyl)-4-(4,4,5,5-tetramethyl-1,3,2-dioxaborolan-2-yl)-1H-pyrazole

To a solution of 1-(2-bromoethyl)-4-(4,4,5,5-tetramethyl-1,3,2-dioxaborolan-2-yl)-1H-pyrazole (100 mg, 332 μmol) in MeCN (3 mL) was added cesium carbonate (540 mg, 1.66 mmol) and 3,3-dimethylazetidine hydrochloride (60.5 mg, 498 μmol). The reaction mixture was refluxed for 4 h. The mixture was cooled to RT, filtered, and the residue was washed with MeCN (2×3 mL). The combined filtrates were concentrated under reduced pressure to give the title product (105 mg, >100%) as a light-yellow solid. Used directly in the next step without further purification. LCMS (Method A): 0.32 min; m/z: 307.1 [M+H]⁺. ¹H NMR (400 MHz, DMSO-d₆): 7.88 (s, 1H), 7.54 (s, 1H), 4.04-4.01 (m, 2H), 2.81-2.80 (m, 4H), 2.72 (t, J=6.4 Hz, 2H), 1.26-1.24 (m, 12H), 1.12 (s, 6H).

### Intermediate C4: N-methyl-2-(4-(4,4,5,5-tetramethyl-1,3,2-dioxaborolan-2-yl)-1H-pyrazol-1-yl)ethan-1-amine

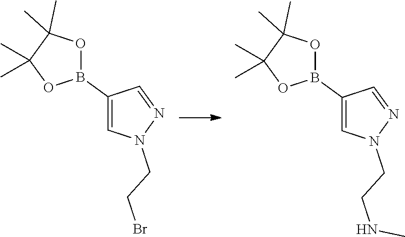

### Step 1: N-methyl-2-(4-(4,4,5,5-tetramethyl-1,3,2-dioxaborolan-2-yl)-1H-pyrazol-1-yl)ethan-1-amine

To a solution of 1-(2-bromoethyl)-4-(4,4,5,5-tetramethyl-1,3,2-dioxaborolan-2-yl)-1H-pyrazole (100 mg, 332 μmol) in MeCN (3 mL) were added methylamine hydrochloride (44.1 mg, 664 μmol), Cs₂CO₃ (540 mg, 1.66 mmol) and KI (5.51 mg, 33.2 μmol) at RT. The reaction mixture was refluxed for 4 h under N₂ atmosphere. The reaction mixture was filtered, and the filtrate was concentrated under reduced pressure to give the title product (118 mg, >100%) as a yellow oil. Used as crude in the next step. LCMS (Method A): 0.30 min; m/z 169.9 [M+H]⁺.

### Intermediate C5: N,N-dimethyl-2-(4-(4,4,5,5-tetramethyl-1,3,2-dioxaborolan-2-yl)-1H-pyrazol-1-yl)ethan-1-amine

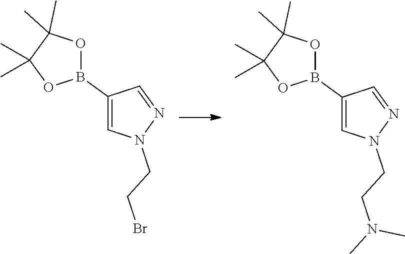

### Step 1: N,N-dimethyl-2-(4-(4,4,5,5-tetramethyl-1,3,2-dioxaborolan-2-yl)-1H-pyrazol-1-yl)ethan-1-amine

To a solution of 1-(2-bromoethyl)-4-(4,4,5,5-tetramethyl-1,3,2-dioxaborolan-2-yl)-1H-pyrazole (100 mg, 0.332 mmol) in MeCN (3 mL) were added dimethylamine (22.4 mg, 0.498 mmol), Cs₂CO₃ (540 mg, 1.66 mmol) and KI (5.51 mg, 0.033 mmol) at RT. The reaction mixture was stirred at 90° C. for 4 h under N₂. The solids were removed by filtration, washed with EtOAc (5 mL) and the combined organics were concentrated under reduced pressure to afford the title product (120 mg, >100%) as a black oil. Used directly as crude in the next step. LCMS (Method A): 0.45 min, m/z 266.1 [M+H]⁺.

### Intermediate C6: 8-methyl-3-[4-(4,4,5,5-tetramethyl-1,3,2-dioxaborolan-2-yl)-1H-pyrazol-1-yl]-8-azabicyclo[3.2.1]octane

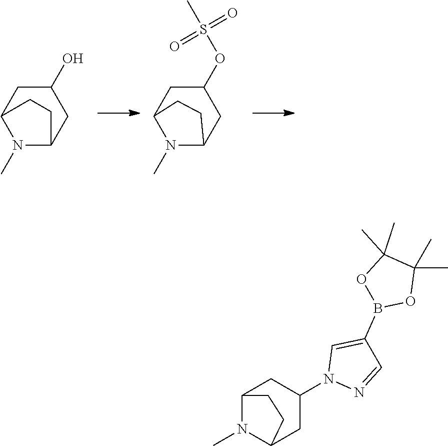

### Step 1: 8-methyl-8-azabicyclo[3.2.1]octan-3-yl methanesulfonate

A mixture of 8-methyl-8-azabicyclo[3.2.1]octan-3-ol (3 g, 21.2 mmol) and Et₃N (5.57 g, 55.1 mmol) in DCM (30 mL) was stirred at 0° C. MsCl (3.14 g, 27.5 mmol) was added at 0° C. and the reaction mixture was stirred at RT for 2 h. The mixture was poured into water (40 mL) and extracted with EtOAc (40 mL×2). The combined organics were dried over Na₂SO₄ and concentrated under reduced pressure to give the crude title product (2.00 g, 9.11 mmol) as an orange solid. Used as crude in the next step. LCMS (Method A): 0.72 min, m/z 219.9 [M+H]⁺.

### Step 2: 8-methyl-3-[4-(4,4,5,5-tetramethyl-1,3,2-dioxaborolan-2-yl)-1H-pyrazol-1-yl]-8-azabicyclo[3.2.1]octane

A mixture of 8-methyl-8-azabicyclo[3.2.1]octan-3-yl methanesulfonate (500 mg, 2.27 mmol), 4-(4,4,5,5-tetramethyl-1,3,2-dioxaborolan-2-yl)-1H-pyrazole (440 mg, 2.27 mmol) and K₂CO₃ (941 mg, 6.81 mmol) in MeCN (10 mL) was stirred at 80° C. for 12 h. The reaction mixture was concentrated under reduced pressure and the residue was poured into water (40 mL) and extracted with EtOAc (40 mL×2). The combined organic phases were dried over Na₂SO₄ and concentrated under reduced pressure. The residue was purified by silica gel column chromatography (PE:EtOAc; 4/1 to 1/4) to give the title product (200 mg, 28%) as a brown solid. LCMS (Method B): 2.58 min, m/z 318.2 [M+H]⁺

### Intermediate C7: 1-methyl-4-(4,4,5,5-tetramethyl-1,3,2-dioxaborolan-2-yl)-1H-pyrrole-2-carbonitrile

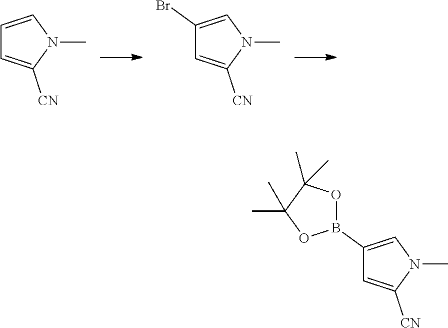

### Step 1: 4-bromo-1-methyl-1H-pyrrole-2-carbonitrile

To a solution of 1-methyl-1H-pyrrole-2-carbonitrile (500 mg, 4.71 mmol) in DMF (5 mL) was added NBS (838 mg, 4.71 mmol). The reaction mixture was stirred at RT overnight. The mixture was poured into water (20 mL) and extracted with EtOAc (50 mL×2). The combined organic phases were dried over Na₂SO₄ and concentrated under reduced pressure. The residue was purified by column chromatography on silica gel (PE:EtOAc; 4:1) to give the title product (700 mg, 80%) as a white solid. ¹H NMR (400 MHz, CDCl₃): 6.81 (s, 1H), 6.76 (s, 1H), 3.77 (s, 3H).

### Step 2: 1-methyl-4-(4,4,5,5-tetramethyl-1,3,2-dioxaborolan-2-yl)-1H-pyrrole-2-carbonitrile

To a solution of 4-bromo-1-methyl-1H-pyrrole-2-carbonitrile (300 mg, 1.62 mmol), B₂pin₂ (431 mg, 1.70 mmol) and AcOK (476 mg, 4.86 mmol) in degassed 1,4-dioxane (10 mL) was added Pd(dppf)Cl₂ (74.1 mg, 81.0 μmol). The mixture was stirred at 100° C. under N₂ overnight. The mixture was concentrated under reduced pressure and the residue was purified by silica gel column chromatography (PE:EtOAc; 2:1) to give the title product (200 mg, 53%) as a white solid. No mass ion

### Intermediate C8: 2-{3-[4-(4,4,5,5-tetramethyl-1,3,2-dioxaborolan-2-yl)-1H-pyrazol-1-yl]oxetan-3-yl)acetonitrile

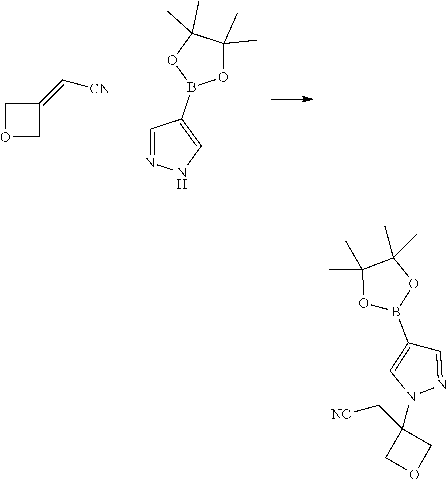 [SMILES: N#CC=C1COC1 ; CC1(C)OB(c2cn[nH]c2)OC1(C)C ; CC1(C)OB(c2cnn(C3(CC#N)COC3)c2)OC1(C)C]

### Step 1: 2-{3-[4-(4,4,5,5-tetramethyl-1,3,2-dioxaborolan-2-yl)-1H-pyrazol-1-yl]oxetan-3-yl}acetonitrile

To a mixture of 2-(oxetan-3-ylidene)acetonitrile (200 mg, 2.10 mmol) and 4-(4,4,5,5-tetramethyl-1,3,2-dioxaborolan-2-yl)-1H-pyrazole (520 mg, 2.68 mmol) in MeCN (15 mL) was added DBU (639 mg, 4.20 mmol) and the reaction mixture was stirred at 60° C. for 18 h. The mixture was concentrated under reduced pressure and the residue was diluted with water (20 mL) and extracted with EtOAc (3×20 mL). The combined organics were washed with brine (50 mL), dried over Na₂SO₄ and concentrated under reduced pressure. The residue was purified by Prep-TLC (PE:EtOAc; 4:1) to give the title product (250 mg, 41%) as a yellow oil. ¹H NMR (400 MHz, DMSO-d₆): 8.30 (s, 1H), 7.76 (s, 1H), 5.01 (d, J=7.2 Hz, 2H), 4.74 (d, J=7.2 Hz, 2H), 3.61 (s, 2H), 1.27 (s, 12H).

### Intermediate C9: N-isopropyl-N-methyl-2-(4-(4,4,5,5-tetramethyl-1,3,2-dioxaborolan-2-yl)-1H-pyrazol-1-yl)propan-1-amine

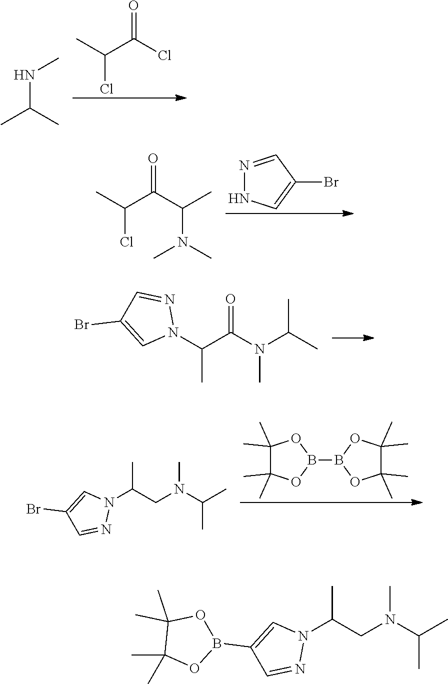

### Step 1: 2-chloro-N-isopropyl-N-methylpropanamide

To a solution of methyl(propan-2-yl)amine (1 g, 13.6 mmol) in DCM (14 mL) was added Et₃N (2.28 g, 22.6 mmol) followed by 2-chloropropanoyl chloride (1.43 g, 11.3 mmol) at 0° C. The solution was stirred at 0° C. for 1 h under N₂. The solution was diluted with EtOAc (50 mL), washed with water (10 mL) and brine (3 mL), dried over Na₂SO₄ and concentrated under reduced pressure to give the title product (1.70 g, 76%) as a white oil. LCMS (Method C): 0.74 min; m/z: 163.9 [M+H]⁺

### Step 2: 2-(4-bromo-1H-pyrazol-1-yl)-N-isopropyl-N-methylpropanamide

To a solution of 2-chloro-N-methyl-N-(propan-2-yl)propanamide (1.56 g, 9.53 mmol) in DMF (25 mL) were added 4-bromo-1H-pyrazole (1.16 g, 7.94 mmol) and K₂CO₃ (3.28 g, 23.8 mmol) at RT. The solution was stirred at 130° C. overnight under N₂. The solution was diluted with water (25 mL) and the organics were extracted with EtOAc (2×10 mL). The combined organics were washed with water (10 mL) and brine (10 mL), dried over Na₂SO₄ and concentrated under reduced pressure. The residue was purified by silica gel column chromatography (PE:EtOAC; 5:1) to give the title product (2.00 g, 76%) as a colourless oil. LCMS (Method C): 1.20 min; m/z: 275.9 [M+H]⁺

### Step 3: 2-(4-bromo-1H-pyrazol-1-yl)-N-isopropyl-N-methylpropan-1-amine

To a solution of 2-(4-bromo-1H-pyrazol-1-yl)-N-methyl-N-(propan-2-yl)propenamide (500 mg, 1.82 mmol) in THE (8 mL) was added LiAlH₄ (138 mg, 3.64 mmol) at RT. The solution was stirred at RT for 1 h under N₂. The solution was diluted with water (25 mL) and the organics were extracted with EtOAC (2×10 mL). The combined organics were washed with water (10 mL) and brine (10 mL), dried over Na₂SO₄ and concentrated under reduced pressure. The residue was purified by silica gel column chromatography (DCM:MeOH; 100/1) to give the title product (205 mg, 43%) as a white solid. LCMS (Method C): 0.29 min; m/z 259.9 [M+H]⁺

### Step 4: methyl(propan-2-yl){2-[4-(4,4,5,5-tetramethyl-1,3,2-dioxaborolan-2-yl)-1H-pyrazol-1-yl]propyl}amine

To a solution of 2-(4-bromo-1H-pyrazol-1-yl)-N-isopropyl-N-methylpropan-1-amine (100 mg, 384 μmol) in 1,4-dioxane (4 mL) were added B₂pin₂ (146 mg, 576 μmol), Pd(dppf)Cl₂ (28.0 mg, 38.4 μmol) and AcOK (94.2 mg, 960 μmol). The solution was stirred at 100° C. for 8 h under N₂. The solution was concentrated and the crude was purified by silica gel column chromatography (PE/EtOAc=1/1) to give the title compound (18.0 mg, 15%) as a white oil. LCMS (method C): 0.36 min; m/z: 307.9 [M+H]⁺

### Intermediate C10: 1-(4-(4,4,5,5-tetramethyl-1,3,2-dioxaborolan-2-yl)-1H-pyrazol-1-yl)cyclopropane-1-carbonitrile

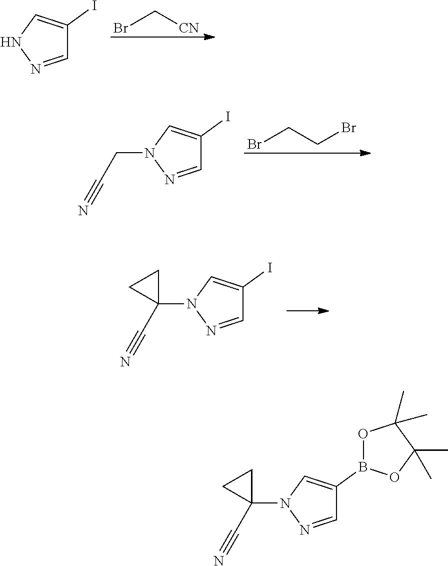

### Step 1: 2-(4-iodo-1H-pyrazol-1-yl)acetonitrile

To a solution of 4-iodo-1H-pyrazole (1 g, 5.15 mmol) in DMF (10 mL) were added K₂CO₃ (1.42 g, 10.3 mmol) and 2-bromoacetonitrile (678 mg, 5.66 mmol) at RT. The reaction mixture was stirred at 50° C. overnight. The reaction mixture was extracted with EtOAc (2×30 mL) and the combined organics were washed with water and brine, dried over Na₂SO₄ and concentrated under reduced pressure. The crude product was purified by silica gel column chromatography (PE:EtOAc; 5:1) to give title product (1.10 g, 92%) as a white solid. ¹HNMR (400 MHz, DMSO-d₆): 8.02 (s, 1H), 7.68 (s, 1H), 5.49 (s, 2H).

### Step 2: 1-(4-iodo-1H-pyrazol-1-yl)cyclopropane-1-carbonitrile

To a solution of 2-(4-iodo-1H-pyrazol-1-yl)acetonitrile (1.1 g, 4.72 mmol) in DMSO (8 mL) was added NaH (60% in oil, 451 mg, 18.8 mmol) at 0° C. and the mixture was stirred at 0° C. for 30 mins. 1,2-dibromoethane (2.64 g, 14.1 mmol) in DMSO (2 mL) was added dropwise and the reaction was stirred at RT for 8 h. The reaction was quenched with water (10 mL) and the organics were extracted with EtOAc (30 mL×2). The combined organics were washed with brine, dried over Na₂SO₄ and concentrated under reduced pressure. The crude product was purified by silica gel column chromatography (PE: EtOAc; 10/1) to give title product (274 mg, 22%) as yellow oil. LCMS (method E): 0.95 min; m/z: 259.9 [M+H]⁺

### Step 3: 1-(4-(4,4,5,5-tetramethyl-1,3,2-dioxaborolan-2-yl)-1H-pyrazol-1-yl)cyclopropane-1-carbonitrile

To a solution of 1-(4-iodo-1H-pyrazol-1-yl)cyclopropane-1-carbonitrile (255 mg, 984 μmol) in degassed DMSO (10 mL) were added B₂(pin)₂ (373 mg, 1.47 mmol), Pd(dppf)Cl₂ (71.9 mg, 98.3 μmol) and AcOK (289 mg, 2.95 mmol) at RT and the reaction mixture was stirred at 80° C. for 3 h. The mixture was partitioned between H₂O (10 mL) and EtOAc (10 mL), and the layers were separated. The aqueous fraction was extracted with EtOAc (30 mL×2). The combined organics were washed with water and brine, dried over Na₂SO₄ and concentrated under reduced pressure. The crude product was purified by silica gel column chromatography (PE: EtOAc; 20/1 to 5/1) to give title product (150 mg, 59%) as a white solid. LCMS (Method E): 1.03 min; m/z: 259.9 [M+H]⁺

### Intermediate C11: 1-(methyl-d3)-4-(4-(4,4,5,5-tetramethyl-1,3,2-dioxaborolan-2-yl)-1H-pyrazol-1-yl)piperidine

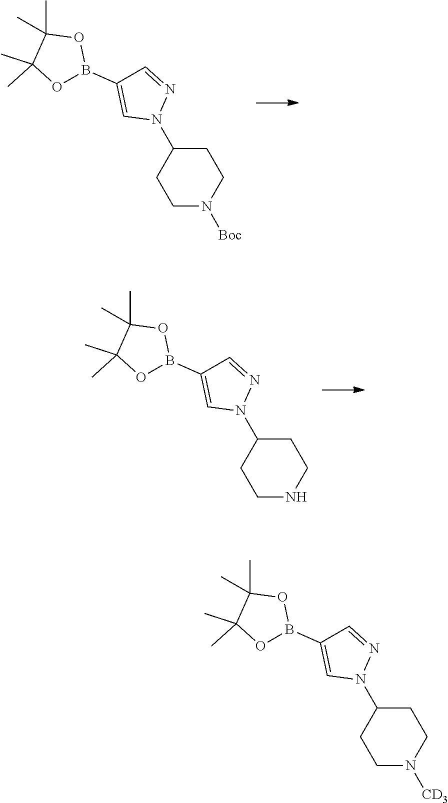 [SMILES: CC(C)(C)OC(=O)N1CCC(n2cc(B3OC(C)(C)C(C)(C)O3)cn2)CC1 ; CC1(C)OB(c2cnn(C3CCNCC3)c2)OC1(C)C ; [2H]C([2H])([2H])N1CCC(n2cc(B3OC(C)(C)C(C)(C)O3)cn2)CC1]

### Step 1: 4-(4˜4,4,5,5-tetramethyl-1,3,2-dioxaborolan-2-yl)-1H-pyrazol-yl)piperidine

A solution of tert-butyl 4-[4-(4,4,5,5-tetramethyl-1,3,2-dioxaborolan-2-yl)-1H-pyrazol-1-yl]piperidine-1-carboxylate (377 mg, 999 μmol) in HCOOH (2 mL) was stirred at RT for 1 h. The mixture was concentrated under reduced pressure to give the title compound as a formate salt (270 mg, 97%) as a colorless oil. LCMS (Method A): 2.17 min, m/z 278.1 [M+H]⁺

### Step 2: 1-(methyl-d3)-4-(4-(4,4,5,5-tetramethyl-1,3,2-dioxaborolan-2-yl)-1H-pyrazol-1-yl)piperidine

To a solution of 4-[4-(4,4,5,5-tetramethyl-1,3,2-dioxaborolan-2-yl)-1H-pyrazol-1-yl]piperidine (270 mg, 974 μmol) in MeCN (5 mL) was added K₂CO₃ (134 mg, 974 μmol) and CD₃I (141 mg, 974 μmol). The reaction mixture was stirred at 80° C. for 2 h. The mixture was poured into water (5 mL) and extracted with EtOAc (3×5 mL). The combined organics were washed with brine, dried over Na₂SO₄ and concentrated under reduced pressure to give the title product (290 mg, >100%) as a white solid. Used as crude in the next step. LCMS (Method A): 2.09 min, m/z 295.1 [M+H]⁺

### Intermediate C12: tert-butyl 3-methyl-5-(4-(4,4,5,5-tetramethyl-1,3,2-dioxaborolan-2-yl)-1H-pyrazol-1-yl)piperidine-1-carboxylate

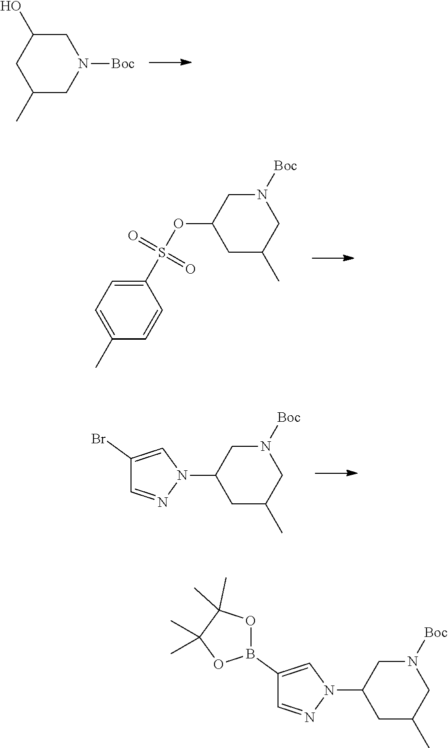 [SMILES: CC1CC(O)CN(C(=O)OC(C)(C)C)C1 ; Cc1ccc(S(=O)(=O)OC2CC(C)CN(C(=O)OC(C)(C)C)C2)cc1 ; CC1CC(n2cc(Br)cn2)CN(C(=O)OC(C)(C)C)C1 ; CC1CC(n2cc(B3OC(C)(C)C(C)(C)O3)cn2)CN(C(=O)OC(C)(C)C)C1]

### Step 1: tert-butyl 3-methyl-5-(tosyloxy)piperidine-1-carboxylate

To a solution of tert-butyl 3-hydroxy-5-methylpiperidine-1-carboxylate (575 mg, 2.67 mmol) in DCM (10 mL) and pyridine (10 mL) was added TsCl (610 mg, 3.20 mmol). After stirring at RT overnight, the reaction mixture was concentrated under reduced pressure. The residue was purified by silica gel column chromatography (PE:EtOAc; 25/1) to give the title product (800 mg, 81%) as a white solid. LCMS (Method A): 4.28 min; 392.2 [M+Na]⁺

### Step 2: tert-butyl 3-(4-bromo-1H-pyrazol-1-yl)-5-methylpiperidine-1-carboxylate

To a solution of tert-butyl 3-methyl-5-(tosyloxy)piperidine-1-carboxylate (500 mg, 1.35 mmol) in DMF (10 mL) were added 4-bromo-1H-pyrazole (257 mg, 1.75 mmol) and Cs₂CO₃ (1.31 g, 4.05 mmol). After stirring at 100° C. overnight under N₂, the reaction mixture was concentrated under reduced pressure. The residue was purified by silica gel column chromatography (PE:EtOAc; 5/1) to give the title product (230 mg, 50%) as a colorless oil. LCMS (Method A): 4.19 min; m/z: 288.0, 290.0 [M+H]⁺

### Step 3: tert-butyl 3-methyl-5-(4-(4,4,5,5-tetramethyl-1,3,2-dioxaborolan-2-yl)-1H-pyrazol-1-yl)piperidine-1-carboxylate

To a solution of tert-butyl 3-(4-bromo-1H-pyrazol-1-yl)-5-methylpiperidine-1-carboxylate (200 mg, 0.581 mmol) in DME (10 mL) were added AcOK (113 mg, 1.16 mmol), B₂pin₂ (221 mg, 871 μmol) and Pd(dppf)Cl₂ (42.4 mg, 58.0 μmol). After stirring at 100° C. overnight under N₂, the mixture was concentrated under reduced pressure. The residue was diluted with water (100 mL) and extracted with EtOAc (100 mL×3). The combined organic phases were washed with water and brine, dried over Na₂SO₄ and concentrated under reduced pressure to give the title product (400 mg, >100%) as a black oil. Used as crude in the next step. LCMS (Method A): 3.50 min; m/z: 392.3 [M+H]⁺

### Intermediate C13: tert-butyl 2-((4-(4,4,5,5-tetramethyl-1,3,2-dioxaborolan-2-yl)-1H-pyrazol-1-yl)methyl)pyrrolidine-1-carboxylate

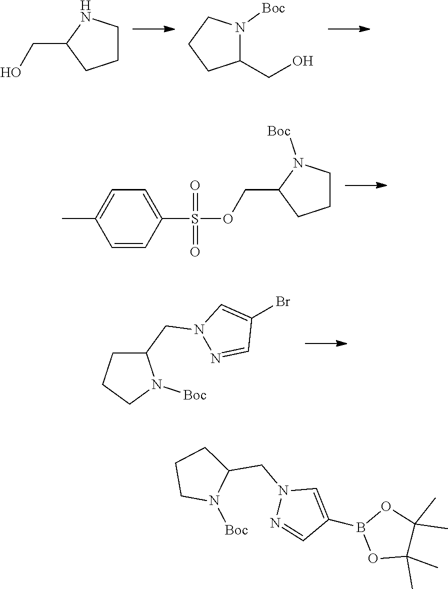 [SMILES: OCC1CCCN1 ; CC(C)(C)OC(=O)N1CCCC1CO ; Cc1ccc(S(=O)(=O)OCC2CCCN2C(=O)OC(C)(C)C)cc1 ; CC(C)(C)OC(=O)N1CCCC1Cn1cc(Br)cn1 ; CC(C)(C)OC(=O)N1CCCC1Cn1cc(B2OC(C)(C)C(C)(C)O2)cn1]

### Step 1: tert-butyl 2-(hydroxymethyl)pyrrolidine-1-carboxylate

To a solution of (pyrrolidin-2-yl)methanol (500 mg, 4.94 mmol) in DCM (5 mL) was added Et₃N (2.49 g, 24.7 mmol) followed by Boc₂O (1.29 g, 5.92 mmol). The reaction mixture was stirred at RT for 16 h. The solvents were removed under reduced pressure and the residue was purified by silica gel column chromatography (PE:EA, 1:1) to give the title product (850 mg, 85%) as a yellow oil.

### Step 2: tert-butyl 2-{[(4-methylbenzenesulfonyl)oxy]methyl}pyrrolidine-1-carboxylate

To a solution of tert-butyl 2-(hydroxymethyl)pyrrolidine-1-carboxylate (850 mg, 4.22 mmol) in DCM (5 mL) were added TsCl (2.40 g, 12.6 mmol) and Et₃N (2.56 g, 25.3 mmol). The mixture was stirred at RT overnight. Water (10 mL) was added and the organics were extracted with DCM (2×10 mL). The combined organics were dried over Na₂SO₄ and concentrated under reduced pressure. The residue was purified by silica gel column chromatography (PE:EA, 4:1) to give the title product (1.40 g, 93%) as a yellow oil.

### Step 3: tert-butyl 2-[(4-bromo-1H-pyrazol-1-yl)methyl]pyrrolidine-1-carboxylate

To a solution of tert-butyl 2-{[(4-methylbenzenesulfonyl)oxy]methyl}pyrrolidine-1-carboxylate (1.4 g, 3.93 mmol) in DMF (10 mL) were added Cs₂CO₃ (3.80 g, 11.7 mmol) and 4-bromo-1H-pyrazole (692 mg, 4.71 mmol). The reaction mixture was stirred at 100° C. for 16 h. Water (10 mL) was added and the organics were extracted with EtOAc (2×20 mL). The combined organics were washed with brine, dried over Na₂SO₄ and concentrated under reduced pressure. The residue was purified by silica gel column chromatography (PE:EA, 2:1) to give the title product (1.30 g, 100%) as a colorless oil.

### Step 4: tert-butyl 2-((4-(4,4,5,5-tetramethyl-1,3,2-dioxaborolan-2-yl)-1H-pyrazol-1-yl)methyl)pyrrolidine-1-carboxylate

To a solution of tert-butyl 2-[(4-bromo-1H-pyrazol-1-yl)methyl]pyrrolidine-1-carboxylate (1.3 g, 3.93 mmol) in DME (10 mL) were added AcOK (1.14 g, 11.7 mmol), B₂(pin)₂ (1.19 g, 4.71 mmol) and Pd(dppf)Cl₂ (287 mg, 393 μmol). The reaction mixture was stirred at 100° C. for 16 h under N₂. The reaction mixture was concentrated under reduced pressure and the residue was purified by silica gel column chromatography (PE:EtOAc, 3:1) to give the title compound (1.0 g, 67%) as a colorless oil. ¹H NMR (400 MHz, DMSO-d₆): 7.82 (s, 1H), 7.58 (s, 1H), 4.22-4.13 (m, 3H), 3.20-3.10 (m, 2H), 1.75-1.69 (m, 4H), 1.41-1.37 (m, 9H), 1.24 (s, 12H)

### Intermediate C14: 6-[4-(4,4,5,5-tetramethyl-1,3,2-dioxaborolan-2-yl)-1H-pyrazol-1-yl]-2-azaspiro[3.3]heptane-2-carboxylate

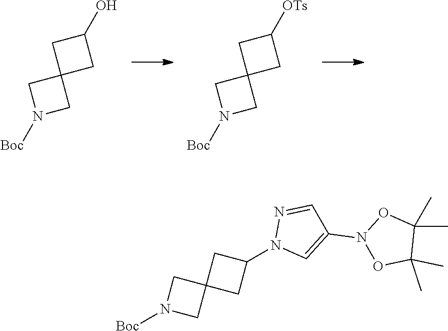 [SMILES: CC(C)(C)OC(=O)N1CC2(CC(O)C2)C1 ; Cc1ccc(S(=O)(=O)OC2CC3(C2)CN(C(=O)OC(C)(C)C)C3)cc1 ; CC(C)(C)OC(=O)N1CC2(CC(n3cc(N4OC(C)(C)C(C)(C)O4)cn3)C2)C1]

### Step 1: tert-butyl 6-[(4-methylbenzenesulfonyl)oxy]-2-azaspiro[3.3]heptane-2-carboxylate

To a solution of tert-butyl 6-hydroxy-2-azaspiro [3.3]heptane-2-carboxylate (3.0 g, 14.0 mmol) in DCM/pyridine (4/1, 100 mL) was added TsCl (2.93 g, 15.4 mmol). The mixture was stirred at RT for 12 h. Water (50 mL) was added and the organics were extracted with EtOAc (3×50 mL). The combined organics were washed with brine (50 mL), dried over Na₂SO₄ and concentrated under reduced pressure. The residue was purified by column chromatography on silica gel (PE:EA; 5:1) to give the title product (3.00 g, 58%) as a white solid.

### Step 2: tert-butyl 6-(4-(4,4,5,5-tetramethyl-1,3,2-dioxaborolan-2-yl)-1H-pyrazol-1-yl)-2-azaspiro[3.3]heptane-2-carboxylate

To a solution of tert-butyl 6-[(4-methylbenzenesulfonyl)oxy]-2-azaspiro[3.3]heptane-2-carboxylate (1.2 g, 3.26 mmol) in DMF (40 mL) were added Cs₂CO₃ (2.12 g, 6.52 mmol) and 4-(4,4,5,5-tetramethyl-1,3,2-dioxaborolan-2-yl)-1H-pyrazole (632 mg, 3.26 mmol), and the mixture was stirred at 100° C. overnight. The solvent was removed under reduced pressure and the residue was purified by silica gel column chromatography (PE:EA; 3:1) to give the title product (800 mg, 63%) as a yellow solid.

### Intermediate C15: tert-butyl 4-[4-(4,4,5,5-tetramethyl-1,3,2-dioxaborolan-2-yl)-1H-pyrazol-1-yl]azepane-1-carboxylate

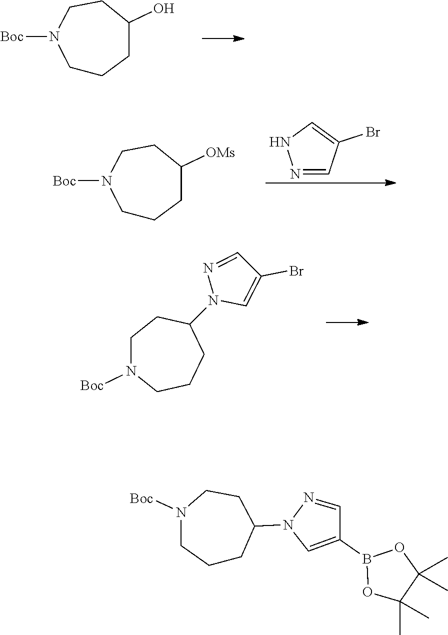 [SMILES: CC(C)(C)OC(=O)N1CCCC(O)CC1 ; CC(C)(C)OC(=O)N1CCCC(OS(C)(=O)=O)CC1 ; Brc1cn[nH]c1 ; CC(C)(C)OC(=O)N1CCCC(n2cc(Br)cn2)CC1 ; CC(C)(C)OC(=O)N1CCCC(n2cc(B3OC(C)(C)C(C)(C)O3)cn2)CC1]

### Step 1: tert-butyl 4-((methylsulfonyl)oxy)azepane-1-carboxylate

To a 0° C. solution of tert-butyl 4-hydroxyazepane-1-carboxylate (860 mg, 3.99 mmol) in DCM (10 mL) were added Et₃N (808 mg, 7.98 mmol) and MsCl (684 mg, 5.98 mol). The reaction was stirred at room temperature for 1 h under N₂. Water (10 mL) was added and the organics were extracted with DCM (3×15 mL). The combined organics were washed with brine, dried over Na₂SO₄ and concentrated under reduced pressure to give the title compound (1.1 g, 94%) as a yellow oil. ¹HNMR: (400 MHz, DMSO-d₆): 3.36-3.32 (m, 3H), 3.22 (brs, 2H), 3.16 (s, 3H), 2.00-1.95 (m, 1H), 1.84-1.77 (m, 4H), 1.64-1.61 (m, 1H), 1.40 (s, 9H).

### Step 2: tert-butyl 4-(4-bromo-1H-pyrazol-1-yl)azepane-1-carboxylate

To a solution of tert-butyl 4-(methanesulfonyloxy)azepane-1-carboxylate (1.1 g, 3.74 mmol) in DMF (15 mL) were added Cs₂CO₃ (3.64 g, 11.2 mmol) and 4-bromo-1H-pyrazole (549 mg, 3.74 mmol) at RT. The reaction mixture was refluxed for 4 h. The solvent was removed under reduced pressure and the residue was purified by silica gel column chromatography (PE:EtOAc; 5:1) to give the title product (1.0 g, 77%) as a yellow oil. LCMS (method A): 1.20 min; m/z 287.7, 289.7 [M+H−tBu]⁺. ¹HNMR: (400 MHz, DMSO-d₆): 8.03 (s, 1H), 7.51 (s, 1H), 4.32-4.26 (m, 1H), 3.60-3.52 (m, 1H), 3.38-3.36 (m, 2H), 3.23-3.17 (m, 1H), 2.03-1.81 (m, 5H), 1.61-1.62 (m, 1H), 1.41 (s, 9H).

### Step 3: tert-butyl 4-[4-(4,4,5,5-tetramethyl-1,3,2-dioxaborolan-2-yl)-1H-pyrazol-1-yl]azepane-1-carboxylate

A mixture of tert-butyl 4-(4-bromo-1H-pyrazol-1-yl)azepane-1-carboxylate (700 mg, 2.03 mmol), AcOK (595 mg, 6.08 mmol), B₂(pin)₂ (771 mg, 3.04 mmol) and Pd(dppf)Cl₂ (147 mg, 202 μmol) in degassed 1,4-dioxane (15 mL) was stirred at 80° C. for 8 h under N₂. The reaction mixture was filtered and the filtrate was concentrated under reduced pressure. The residue was purified by silica gel column chromatography (PE:EtOAc; 5:1) to give the title compound (700 mg, 1.78 mmol) as a yellow oil. LCMS (method A): 1.80 min; m/z 391.9 [M+H]⁺

### Intermediate C16: 1-(1-methylpyrrolidin-3-yl)-4-(4,4,5,5-tetramethyl-1,3,2-dioxaborolan-2-yl)-1H-pyrazole

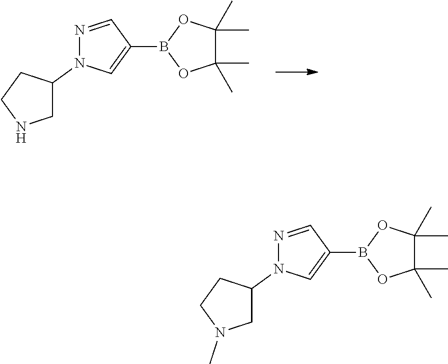 [SMILES: CC1(C)OB(c2cnn(C3CCNC3)c2)OC1(C)C ; CN1CCC(n2cc(B3OC(C)(C)C(C)(C)O3)cn2)C1]

### Step 1: 1-(1-methylpyrrolidin-3-yl)-4-(4,4,5,5-tetramethyl-1,3,2-dioxaborolan-2-yl)-1H-pyrazole

A mixture of 1-(pyrrolidin-3-yl)-4-(4,4,5,5-tetramethyl-1,3,2-dioxaborolan-2-yl)-1H-pyrazole (200 mg, 760 μmol), formaldehyde (184 mg, 2.28 mmol) and NaBH(OAc)₃ (1.28 g, 6.07 mmol) in DCM (10 mL) was stirred at RT overnight. The mixture was poured into water and extracted with EtOAc (50 mL×3). The combined organics were dried over Na₂SO₄ and concentrated under reduced pressure. The residue was purified by column chromatography on silica gel (PE/EA: 95/5) to give the title product (140 mg, 67%) as a yellow oil. LCMS (Method A): 3.04 min; m/z 278.2 [M+H]⁺

### Intermediate C17: 1-(1-isopropylazetidin-3-yl)-4-(4,4,5,5-tetramethyl-1,3,2-dioxaborolan-2-yl)-1H-pyrazole

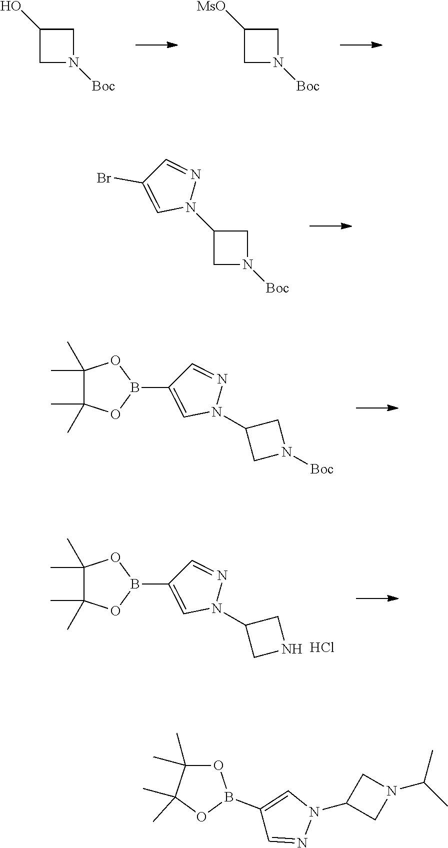

### Step 1: tert-butyl 3-((methylsulfonyl)oxy)azetidine-1-carboxylate

To a mixture of tert-butyl 3-hydroxyazetidine-1-carboxylate (5.0 g, 28.9 mmol) and Et₃N (8.75 g, 86.60 mmol) in DCM (50 mL) at 0° C. was added MsCl (4.94 g, 43.30 mmol) and the reaction was stirred at 0° C. for 3 h. Water (20 mL) was added and the organics were extracted with DCM (2×20 mL). The combined organics were washed with brine, dried over Na₂SO₄ and concentrated under reduced pressure to give the title compound (7.2 g, >100%) as a white solid. LCMS (Method C): 1.07 min; m/z 195.8 [M+H-tbu]⁺

### Step 2: tert-butyl 3-(4-bromo-1H-pyrazol-1-yl)azetidine-1-carboxylate

To a solution of tert-butyl 3-((methylsulfonyl)oxy)azetidine-1-carboxylate (7.2 g, 28.65 mmol) in DMF (200 ml) were added Cs₂CO₃ (27.9 g, 85.95 mmol) and 4-bromo-1H-pyrazole (4.63 g, 31.51 mmol) and the reaction mixture was stirred at 80° C. for 3 h. Water (200 mL) was added and the organics were extracted with EtOAc (2×100 mL). The combined organics were washed with brine, dried over Na₂SO₄ and concentrated under reduced pressure. The residue was purified by silica gel column chromatography (PE:EtOAc; 5/1) to give the title compound (7.7 g). LCMS (Method C): 0.79 min; m/z 245.0 [M+H-tbu]⁺

### Step 3: tert-butyl 3-(4-(4,4,5,5-tetramethyl-1,3,2-dioxaborolan-2-yl)-1H-pyrazol-1-yl)azetidine-1-carboxylate

To a mixture of tert-butyl 3-(4-bromo-1H-pyrazol-1-yl)azetidine-1-carboxylate (7.7 g, 25.48 mmol), Pd(dppf)Cl₂ (1.86 g, 2.55 mmol) and AcOK (7.49 g, 76.44 mmol) in degassed 1,4-dioxane (500 mL) was added B₂(pin)₂ (9.71 g, 38.22 mmol) and the reaction was stirred at 100° C. for 6 h under N₂. The reaction mixture was concentrated under reduced pressure and the residue was purified by silica gel column chromatography (PE:EtOAc; 3:1) to give the title compound (8.2 g) as a colorless oil. LCMS (Method C): 1.04 min; m/z 293.5 [M+H-tbu]⁺

### Step 4: 1-(azetidin-3-yl)-4-(4,4,5,5-tetramethyl-1,3,2-dioxaborolan-2-yl)-1H-pyrazole hydrochloride

To a solution of tert-butyl 3-(4-(4,4,5,5-tetramethyl-1,3,2-dioxaborolan-2-yl)-1H-pyrazol-1-yl)azetidine-1-carboxylate (8.2 g, 23.47 mmol) in 1,4-dioxane (10 mL) was added HCl (4 M in 1,4-dioxane; 20 mL) and the reaction mixture was stirred at RT for 4 h. The reaction mixture was concentrated under reduced pressure to afford the title product (6.7 g) as a yellow oil. LCMS (Method C): 0.76 min; m/z 250.2 [M+H]⁺

### Step 5: 1-(1-isopropylazetidin-3-yl)-4-(4,4,5,5-tetramethyl-1,3,2-dioxaborolan-2-yl)-1H-pyrazole

To a solution of 1-(azetidin-3-yl)-4-(4,4,5,5-tetramethyl-1,3,2-dioxaborolan-2-yl)-1H-pyrazole (500 mg, 2.00 mmol) in MeCN (10 mL) were added 2-iodopropane (1.01 g, 6.00 mmol) and K₂CO₃ (276 mg, 2.00 mmol) and the reaction was stirred at 45° C. overnight. Water (10 mL) was added and the organics were extracted with EtOAc (2×40 mL). The combined organics were washed with water and brine, dried over Na₂SO₄ and concentrated under reduced pressure. The residue was purified by silica gel column chromatography (DCM:MeOH; 50/1) to give the title product (70 mg) as a colorless oil. LCMS (Method C): 0.44 min; m/z 292.1 [M+H]⁺

### Intermediate C18: 1-(1-methylazetidin-3-yl)-4-(4,4,5,5-tetramethyl-1,3,2-dioxaborolan-2-yl)-1H-pyrazole

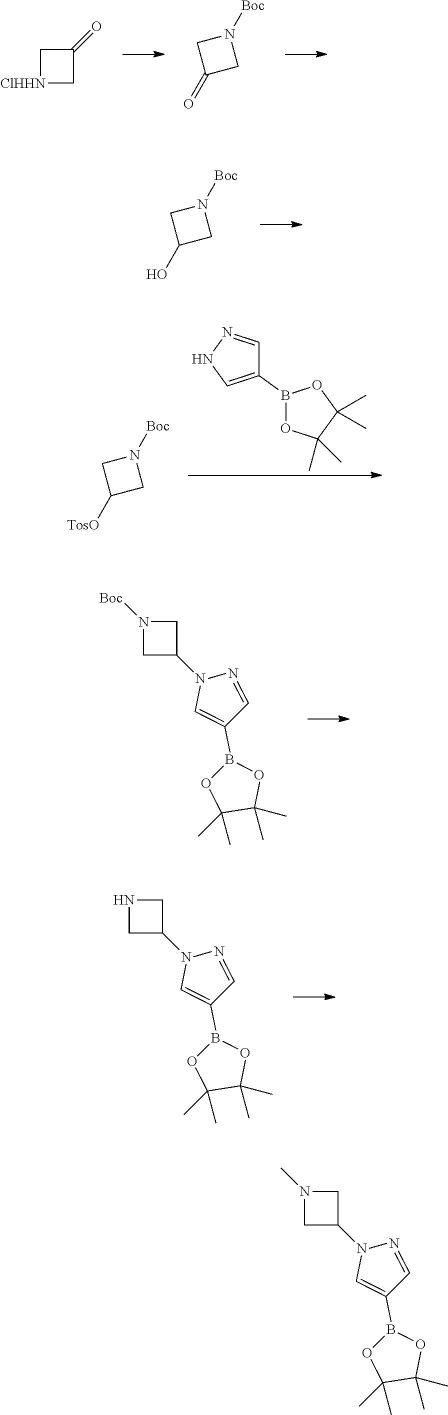

### Step 1: tert-butyl 3-oxoazetidine-1-carboxylate

A mixture of azetidin-3-one hydrochloride (1 g, 9.29 mmol), Boc₂O (3.03 g, 13.9 mmol) and Et₃N (2.81 g, 27.8 mmol) in DCM (40 mL) was stirred at RT overnight. The mixture was washed with brine (2×20 mL) and concentrated under reduced pressure to give the title product (1.17 g, 73.5%) as a yellow oil. ¹H NMR (400 MHz, DMSO-d₆): 4.67 (s, 4H), 1.42 (s, 9H).

### Step 2: tert-butyl 3-hydroxyazetidine-1-carboxylate

A mixture of tert-butyl 3-oxoazetidine-1-carboxylate (6.77 g, 39.5 mmol) and NaBH₄ (2.98 g, 79.0 mmol) in MeOH (80 mL) was stirred at RT for 30 min. The mixture was concentrated under reduced pressure to give the title product (4.30 g, 63%) as a yellowish oil. ¹H NMR (400 MHz, DMSO-d₆): 5.62 (d, J=6.5 Hz, 1H), 4.36 (qt, J=6.7, 4.5 Hz, 1H), 4.03-3.92 (m, 2H), 3.57 (dd, J=9.2, 4.5 Hz, 2H), 1.36 (s, 9H).

### Step 3: tert-butyl 3-[(4-methylbenzenesulfonyl)oxy]azetidine-1-carboxylate

A mixture of tert-butyl 3-hydroxyazetidine-1-carboxylate (2 g, 11.5 mmol), TsCl (2.28 g, 12.0 mmol) and Et₃N (2.32 g, 23.0 mmol) in DCM (5 mL) was stirred at RT overnight. The reaction mixture was washed with brine (50 mL×2), dried over Na₂SO₄ and concentrated under reduced pressure to give the title product (1.25 g, 33%) as a colorless oil. ¹H NMR (400 MHz, DMSO-d₆): 7.81 (d, J=8.4 Hz, 2H), 7.50 (d, J=8.2 Hz, 2H), 5.08 (tt, J=6.6, 3.9 Hz, 1H), 4.13-4.00 (m, 2H), 3.81-3.67 (m, 2H), 2.43 (s, 3H), 1.34 (s, 9H).

### Step 4: tert-butyl 3-[4-(4,4,5,5-tetramethyl-1,3,2-dioxaborolan-2-yl)-1H-pyrazol-1-yl]azetidine-1-carboxylate

A mixture of tert-butyl 3-[(4-methylbenzenesulfonyl)oxy]azetidine-1-carboxylate (1.45 g, 4.42 mmol), Cs₂CO₃ (2.88 g, 8.84 mmol) and 4-(4,4,5,5-tetramethyl-1,3,2-dioxaborolan-2-yl)-1H-pyrazole (1.28 g, 6.63 mmol) in DMF (40 mL) was stirred at 110° C. overnight. The mixture was poured into water (150 mL) and extracted with EtOAc (2×100 mL). The combined organics were washed with brine (2×100 mL), dried over Na₂SO₄ and concentrated under reduced pressure. The residue was purified by silica gel column chromatography (PE:EtOAc; 10:1) to give the title product (750 mg, 49%) as a yellow oil. ¹H NMR (400 MHz, DMSO-d₆): 8.07 (s, 1H), 7.70 (s, 1H), 5.22 (ddd, J=13.2, 8.0, 5.3 Hz, 1H), 4.26 (t, J=8.3 Hz, 2H), 4.11 (s, 2H), 1.40 (s, 9H), 1.25 (s, 12H).

### Step 5: 1-(azetidin-3-yl)-4-(4,4,5,5-tetramethyl-1,3,2-dioxaborolan-2-yl)-1H-pyrazole

A mixture of tert-butyl 3-[4-(4,4,5,5-tetramethyl-1,3,2-dioxaborolan-2-yl)-1H-pyrazol-1-yl]azetidine-1-carboxylate (500 mg, 1.43 mmol) in aqueous HCl (1 M, 2 mL) was stirred at RT for 1 h. The reaction mixture was concentrated under reduced pressure to give the title product (350 mg, 1.40 mmol, 98.3%) as a yellow oil. LCMS (Method A): 1.03 min, m/z 250.0 [M+H]⁺.

### Step 6: 1-(1-methylazetidin-3-yl)-4-(4,4,5,5-tetramethyl-1,3,2-dioxaborolan-2-yl)-1H-pyrazole

A mixture of 1-(azetidin-3-yl)-4-(4,4,5,5-tetramethyl-1,3,2-dioxaborolan-2-yl)-1H-pyrazole (500 mg, 1.40 mmol), paraformaldehyde (337 mg, 4.19 mmol) and NaBH(OAc)₃ (2.37 g, 11.2 mmol) in DCM (30 mL) was stirred at RT for 45 min. The solution was diluted with DCM (30 mL) before the addition of sat. aq. NaHCO₃ (30 mL). The aqueous layer was extracted with a DCM/MeOH mixture (9:1; 3×10 mL). The combined organic layers were concentrated under reduced pressure to give the title product (70 mg, 19%) as a yellow solid. LCMS (Method A): 1.39 min, m/z 264.1 [M+H]⁺.

### Intermediate C19: 4-(4,4,5,5-tetramethyl-1,3,2-dioxaborolan-2-yl)-1-(1-(2,2,2-trifluoroethyl)azetidin-3-yl)-1H-pyrazole

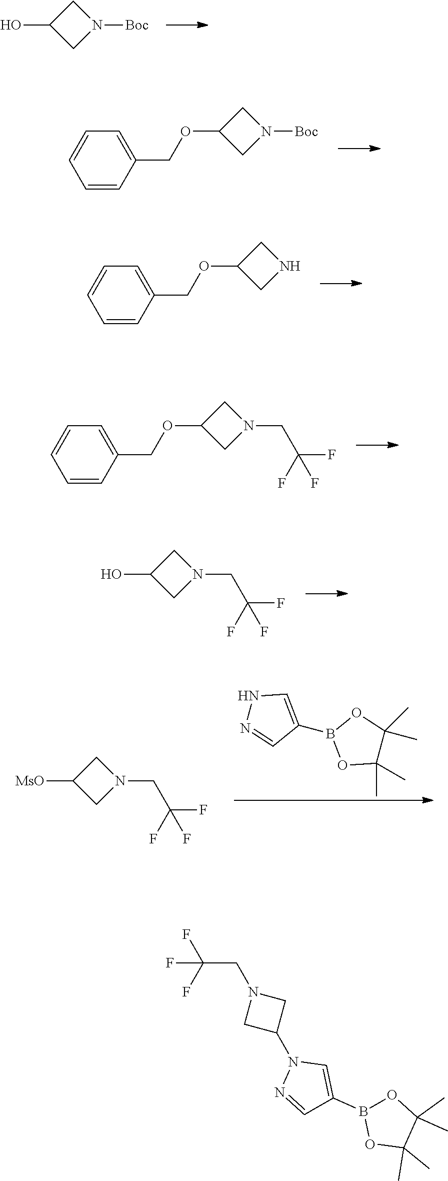

### Step 1: tert-butyl 3-(benzyloxy)azetidine-1-carboxylate

To a 0° C. solution of tert-butyl 3-hydroxyazetidine-1-carboxylate (5 g, 28.9 mmol) in DMF (50 mL) was added NaH (60% in oil, 2.3 g, 57.8 mmol). The solution was stirred at 0° C. for 1 h. benzyl chloride (4.4 g, 34.7 mmol) was added to the solution at 0° C. and the reaction mixture was stirred at RT for 2 h. Water (250 mL) was added and the organics were extracted with EtOAc (2×300 mL). The combined organic layers were washed with brine (500 mL), dried over Na₂SO₄ and concentrated under reduced pressure to afford the title product (7.1 g, 93%) as a white solid. LCMS (Method C): 1.53 min; m/z 208 [M+H−tBu]⁺

### Step 2: 3-(benzyloxy)azetidine

To a solution of tert-butyl 3-(benzyloxy)azetidine-1-carboxylate (4.8 g, 18.2 mmol) in DCM (30 mL) was added TFA (10 mL) and the solution was stirred at RT for 2 h. The solution was concentrated under reduced pressure and the residue was dissolved in MeOH (3 mL). The pH was adjust to pH=8 with the addition of sat. aq. NaHCO₃. The organics were extracted with DCM (2×50 mL), and the combined organics were washed with brine (100 mL), dried over Na₂SO₄ and concentrated under reduced pressure to give the title product (4.10 g, >100%) as a colorless oil. Used as crude in the next step. ¹H NMR (400 MHz, DMSO-d₆): 7.36-7.35 (m, 5H), 4.46 (s, 2H), 4.43-4.38 (m, 1H), 4.03-3.98 (m, 2H), 3.77-3.74 (m, 2H).

### Step 3: 3-(benzyloxy)-1-(2,2,2-trifluoroethyl)azetidine

To a solution of 3-(benzyloxy)azetidine (200 mg, 1.22 mmol) and 1,1,1-trifluoro-2-iodoethane (768 mg, 3.66 mmol) in DMF (3 mL) was added K₂CO₃ (337 mg, 2.44 mmol) and the reaction was stirred at 130° C. for 90 min. Water (20 mL) was added, and the organics were extracted with EtOAc (3×20 mL). The combined organics were washed with water (50 mL) and brine (50 mL), dried over Na₂SO₄ and concentrated under reduced pressure. The residue was purified by silica gel column chromatography (PE:EtOAc; 10:1) to give the title compound (80 mg, 27%) as a light yellow oil. LCMS (Method A): 1.82 min; m/z: 246.0 [M+H]⁺. ¹H NMR (400 MHz, DMSO-d₆): 7.37-7.29 (m, 5H), 4.39 (s, 2H), 4.19-4.15 (m, 1H), 3.60-3.57 (m, 2H), 3.22-3.08 (m, 4H).

### Step 4: 1-(2,2,2-trifluoroethyl)azetidin-3-ol

To a solution of 3-(benzyloxy)-1-(2,2,2-trifluoroethyl)azetidine (130 mg, 530 μmol) in MeOH (10 mL) was added Pd(OH)₂ (37.2 mg, 265 μmol) and the reaction mixture was stirred under H₂ overnight. The mixture was filtered over Celite, and the filtrate was concentrated under reduced pressure to afford the title product (80.0 mg, 97%) as a colorless oil. ¹H NMR (400 MHz, DMSO-d₆): 5.37 (s, 1H), 4.23-4.19 (m, 1H), 3.61-3.58 (m, 2H), 3.17-3.09 (m, 2H), 3.00-2.96 (m, 2H).

### Step 5: 1-(2,2,2-trifluoroethyl)azetidin-3-yl methanesulfonate

To a 0° C. solution of 1-(2,2,2-trifluoroethyl)azetidin-3-ol (80 mg, 515 μmol) in DCM (5 mL) were added Et₃N (103 mg, 1.02 mmol) and MsCl (70.7 mg, 618 μmol). The solution was stirred at RT for 1 h, then poured into water (10 mL) and extracted with DCM (2×20 mL). The combined organic phases were dried over Na₂SO₄ and concentrated under reduced pressure to give the title product (100 mg, 83%) as a light yellow oil. LCMS (Method C): 0.72 min; m/z: 233.8 [M+H]⁺

### Step 6: 4-(4,4,5,5-tetramethyl-1,3,2-dioxaborolan-2-yl)-1-[1-(2,2,2-trifluoroethyl)azetidin-3-yl]-1H-pyrazole

To a solution of 1-(2,2,2-trifluoroethyl)azetidin-3-yl methanesulfonate (500 mg, 2.14 mmol) in DMF (2 mL) were added Cs₂CO₃ (1.04 g, 3.21 mmol), KI (935 mg, 0.21 mmol) and 4-(4,5,5-tetramethyl-1,3,2-dioxaborolan-2-yl)-1H-pyrazole (372 mg, 1.92 mmol), and the reaction was stirred at 80° C. for 2 h. Water (10 mL) was added and the organics were extraxted with EtOAc (2×10 mL). The combined organics were washed with brine, dried over Na₂SO₄ and concentrated under reduced pressure. The residue was purified by silica gel column chromatography (PE:EA; 5:1 to 1:1) to give the title product (130 mg, 20%) as a colourless oil. LCMS (Method C): 1.25 min; m/z: 331.9 [M+H]⁺

### Intermediate C20: N-methyl-N-(propan-2-yl)-2-[4-(4,4,5,5-tetramethyl-1,3,2-dioxaborolan-2-yl)-1H-pyrazol-1-yl]propenamide

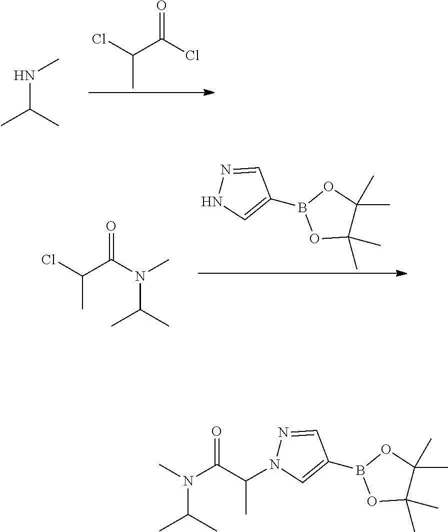

### Step 1: Give 2-chloro-N-methyl-N-(propan-2-yl) propanamide

To a solution of methyl (propan-2-yl)amine (1 g, 13.6 mmol) in DCM (14 mL) were added Et₃N (2.28 g, 22.6 mmol) and 2-chloropropanoyl chloride (1.43 g, 11.3 mmol) at 0° C. The solution was stirred at 0° C. for 1 h under N₂. The solution was partitioned between water (20 ml) and DCM (20 mL), then the organics were collected, wash with brine (10 mL), dried over Na₂SO₄ and concentrated under reduced pressure. The residue was purified by silica gel column chromatography (PE/EtOAc; 5/1) to give the title product (1.46 g, 66%) as a white oil.

### Step 2: N-methyl-N-(propan-2-yl)-2-[4-(4,4,5,5-tetramethyl-1,3,2-dioxaborolan-2-yl)-1H-pyrazol-1-yl]propanamide

To a solution of 2-chloro-N-methyl-N-(propan-2-yl)propanamide (1.45 g, 8.86 mmol) in DMF (25 mL) were added K₂CO₃ (3.05 g, 22.1 mmol) and 4-(4,4,5,5-tetramethyl-1,3,2-dioxaborolan-2-yl)-1H-pyrazole (1.43 g, 7.38 mmol). The solution was stirred at 130° C. overnight under N₂. Water (20 mL) was added and the organics were extracted with EtOAC (2×10 mL). The combined organics were washed with brine (10 mL), dried over Na₂SO₄ and concentrated under reduced pressure. The residue was purified by silica gel column chromatography (PE/EtOAC, 5/1) to give the title product (1.46 g, 51%) as a white solid.

### Intermediate C21: tert-butyl 2,2-dimethyl-5-(4-(4,4,5,5-tetramethyl-1,3,2-dioxaborolan-2-yl)-1H-pyrazol-1-yl)piperidine-1-carboxylate

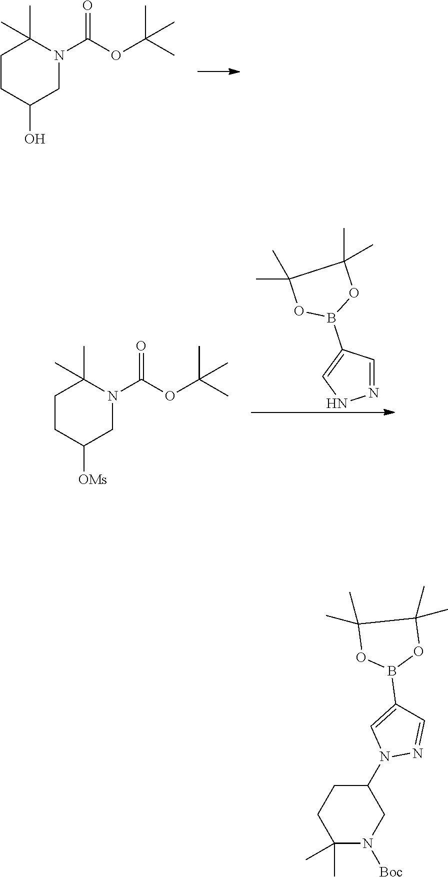

### Step 1: tert-butyl 2,2-dimethyl-5-((methylsulfonyl)oxy)piperidine-1-carboxylate

To a mixture of tert-butyl 5-hydroxy-2,2-dimethylpiperidine-1-carboxylate (50 mg, 218 μmol) in DCM (5 mL) were added Et₃N (51.7 mg, 654 μmol) and MsCl (29.8 mg, 261 μmol), and the mixture was stirred at RT overnight. The mixture was poured into water (10 mL) and the organics were extracted with EtOAc (3×10 mL). The combined organics were washed with brine (10 mL) and concentrated under reduced pressure to give the title compound (60.0 mg, 89%) as a colorless oil.

### Step 2: tert-butyl 2,2-dimethyl-5-(4-(4,4,5,5-tetramethyl-1,3,2-dioxaborolan-2-yl)-1H-pyrazol-1-yl)piperidine-1-carboxylate

A mixture of tert-butyl 2,2-dimethyl-5-((methylsulfonyl)oxy)piperidine-1-carboxylate (370 mg, 1.20 mmol), 4-(4,4,5,5-tetramethyl-1,3,2-dioxaborolan-2-yl)-1H-pyrazole (242 mg, 1.25 mmol) and Cs₂CO₃ (779 mg, 2.40 mmol) in DMF (15 mL) was stirred at 90° C. overnight. The mixture was poured into water (20 mL) and extracted with EtOAc (10 mL×2). The combined organic phases were dried over Na₂SO₄ and concentrated. The residue was purified by prep-TLC (DCM/MeOH=99/1) to give the title product as a yellow solid.

### Intermediate C22: N,N-dimethyl-3-[4-(4,4,5,5-tetramethyl-1,3,2-dioxaborolan-2-yl)-1H-pyrazol-1-yl]cyclopentan-1-amine

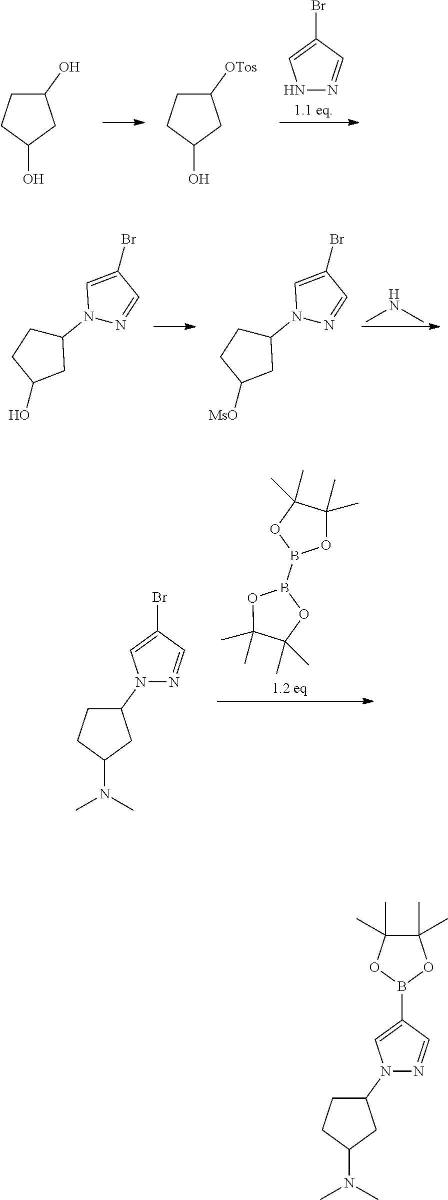

### Step 1: 3-hydroxycyclopentyl 4-methylbenzene-1-sulfonate

A mixture of cyclopentane-1,3-diol (1 g, 9.79 mmol), Et₃N (3.95 g, 39.1 mmol) and TsCl (1.94 g, 10.2 mmol) in DCM (5 mL) was stirred at 0° C. for 2 h. The mixture was diluted with H₂O (100 mL) and extracted with EtOAc (3×200 mL). The combined organic layers were dried over Na₂SO₄ and concentrated under reduced pressure. The residue was purified by silica gel column chromatography (PE:EtOAc=2:1) to afford the title product (1.20 g, 48%) as a yellow oil. LCMS (Method A): 2.93 min, m/z 279.0 [M+Na]⁺

### Step 2: 3-(4-bromo-1H-pyrazol-1-yl)cyclopentan-1-ol

A mixture of 3-hydroxycyclopentyl 4-methylbenzene-1-sulfonate (1.2 g, 4.68 mmol), Cs₂CO₃ (2.77 g, 4.68 mmol) and 4-bromo-1H-pyrazole (687 mg, 4.68 mmol) in DMF (30 mL) was stirred at 50° C. overnight. The reaction mixture was concentrated under reduced pressure, and the residue was diluted with water (30 mL) and extracted with EtOAc (3×30 mL). The combined organic phases were dried over Na₂SO₄ and concentrated under reduced pressure. The residue was purified by Prep-TLC (DCM/MeOH=20/1) to give the title product (750 mg, 69%) as a yellow solid. LCMS (Method A): 4.25 min; m/z 231.0 [M+H]⁺

### Step 3: 3-(4-bromo-1H-pyrazol-1-yl)cyclopentyl 4-methylbenzene-1-sulfonate

A mixture of 3-(4-bromo-1H-pyrazol-1-yl)cyclopentan-1-ol (750 mg, 3.24 mmol), Et₃N (1.30 g, 12.9 mmol) and TsCl (739 mg, 3.88 mmol) in DCM (5 mL) was stirred at 0° C. for 2 h. The mixture was diluted with H₂O (100 mL) and extracted with EtOAc (3×200 mL). The combined organic layers were dried over Na₂SO₄ and concentrated under reduced pressure. The residue was purified by silica gel column chromatography (PE:EtOAc=2:1) to afford the title product (100 mg, 8%) as a yellow oil. LCMS (Method A): 2.93 min, m/z 386.0 [M+H]⁺.

### Step 4: 3-(4-bromo-1H-pyrazol-1-yl)-N,N-dimethylcyclopentan-1-amine

To a mixture of 3-(4-bromo-1H-pyrazol-1-yl)cyclopentyl 4-methylbenzene-1-sulfonate (100 mg, 259 μmol) and K₂CO₃ (253 mg, 777 μmol) in DMF (3 mL) was added dimethylamine (1.2 eq) and the mixture was stirred at 80° C. overnight. The reaction mixture was concentrated under reduced pressure, and the residue was diluted with water (30 mL) and extracted with EtOAc (3×30 mL). The combined organic phases were dried over Na₂SO₄ and concentrated under reduced pressure. The residue was purified by Prep-TLC (DCM/MeOH=20/1) to give the title product (110 mg, >100%) as a yellow solid. LCMS (Method B): 1.00 min; m/z 258.0 [M+H]⁺.

### Step 5: N,N-dimethyl-3-[4-(4,4,5,5-tetramethyl-1,3,2-dioxaborolan-2-yl)-1H-pyrazol-1-yl]cyclopentan-1-amine

A mixture of 3-(4-bromo-1H-pyrazol-1-yl)-N,N-dimethylcyclopentan-1-amine (110 mg, 426 μmol), B₂(pin)₂ (129 mg, 511 μmol), Pd(dppf)Cl₂ (31.1 mg, 42.6 μmol) and AcOK (83.6 mg, 852 μmol) in degassed 1,4-dioxane (2 mL) was stirred at 100° C. under N₂ overnight. The mixture was concentrated under reduced pressure and the residue was purified by Prep-TLC (DCM/MeOH=20/1) to give the title product (60 mg, 46%) as a yellow solid. LCMS (Method B): 3.5 min; m/z 306.0 [M+H]⁺.

### General Procedure A3 for the Suzuki Reaction Between Intermediate A3B1 and Boronate Esters

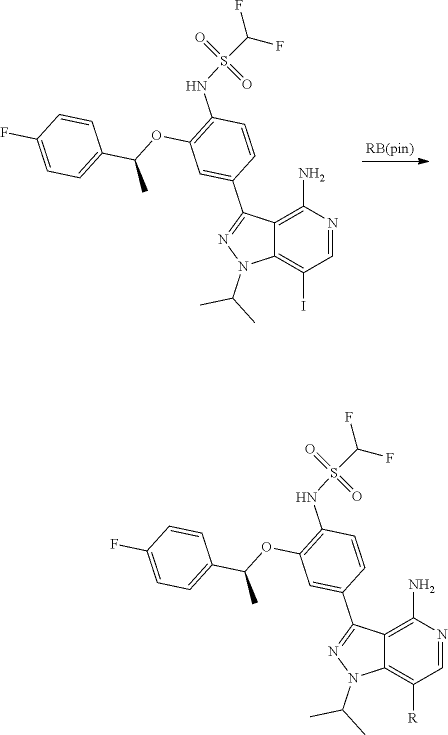

A mixture of (S)—N-(4-(4-amino-7-iodo-1-isopropyl-1H-pyrazolo[4,3-c]pyridin-3-yl)-2-(1-(4-fluorophenyl)ethoxy)phenyl)-1,1-difluoromethanesulfonamide (1 eq), boronate ester (1.1 eq), Na₂CO₃ (3 eq) and Pd(dppf)Cl₂ (0.1 eq) in degassed 1,4-dioxane/H₂O (4/1, 0.2 M) was stirred at 100° C. overnight under N₂. The mixture was diluted with water and the organics were extracted with EtOAc (3×). The combined organics were dried over Na₂SO₄ and concentrated under reduced pressure. The residue was purified by column chromatography (DCM/MeOH) to give the title product as a yellow solid.

The following compounds were prepared following the General procedure A3

| Com- | | | | LCMS | | Boronate |
|---|---|---|---|---|---|---|
| pound | Name | Structure | Yield | data | ¹H NMR data | ester |
| 50 | (S)-N-(4-(4-amino- 1-isopropyl-7-(pyridin- 4-yl)-1H-pyrazolo[4,3- c]pyridin-3-yl)-2-(1- (4-fluorophenyl) ethoxy)phenyl)-1,1- difluoromethane- sulfonamide | 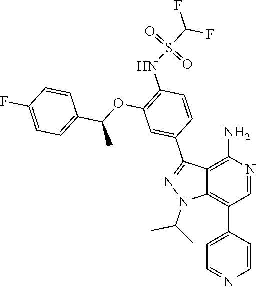 [SMILES: CC(C)n1nc(-c2ccc(NS(=O)(=O)C(F)F)c(O[C@@H](C)c3ccc(F)cc3)c2)c2c(N)ncc(-c3ccncc3)c21] | 55% | (Method A): 3.07 min, m/z: 597.2 [M + H]⁺. | (400 MHz, DMSO-d₆): 8.68 (d, J = 5.6 Hz, 2H), 7.59-7.56 (m, 3H), 7.53 (d, J = 5.6 Hz, 2H), 7.42 (d, J = 8.0 Hz, 1H), 7.18 (t, J = 8.8 Hz, 3H), 7.14 (t, J = 52.4 Hz, 1H), 6.14 (s, 2H), 5.64 (q, J = 6.0 Hz, 1H), 4.27-4.20 (m, 1H), 1.60 (d, J = 6.4 Hz, 3H), 1.24 (dd, J = 2.4, 2.0 Hz, 6H). | 4-(4,4,5,5- tetramethyl- 1,3,2- dioxa- borolan-2- yl) pyridine |
| 51 | (S)-N-(4-(4-amino- 1-isopropyl-7-(1- (tetrahydro-2H-pyran- 4-yl)-1H-pyrazol-4- yl)-1H-pyrazolo[4,3-c] pyridin-3-yl)-2-(1-(4- fluorophenyl)ethoxy) phenyl)-1,1- difluoromethane- sulfonamide | 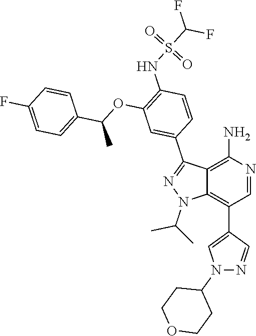 [SMILES: CC(C)n1nc(-c2ccc(NS(=O)(=O)C(F)F)c(O[C@@H](C)c3ccc(F)cc3)c2)c2c(N)ncc(-c3cnn(C4CCOCC4)c3)c21] | 19% | (Method A): 3.28 min, m/z: 670.2 [M + H]⁺. | (400 MHz, DMSO-d₆): 8.05 (s, 1H), 7.62 (s, 1H), 7.57 (t, J = 5.6 Hz, 3H), 7.53 (s, 1H), 7.41 (d, J = 8.0 Hz, 1H), 7.19-7.14 (m, 4H), 7.11 (s, 1H), 7.03 (t, J = 52.4 Hz, 1 H), 6.21 (s, 2H), 5.63 (q, J = 5.2 Hz, 1H), 4.53-4.44 (m, 1H), 3.98 (d, J = 11.2 Hz, 2H), 5.22 (d, J = 10.8 Hz, 2H), 2.02 (s, 4H), 1.59 (d, J = 6.0 Hz, 3H), 1.27 (d, J = 5.6 Hz, 6H). | 1- (tetrahydro- 2H-pyran- 4-yl)-4- (4,4,5,5- tetramethyl- 1,3,2-dioxa- borolan-2- yl)-1H- pyrazole |
| 52 | (S)-N-(4-(4-amino- 1-isopropyl-7-(1- methyl-1H-pyrazol-4- yl)-1H-pyrazolo[4,3- c]pyridin-3-yl)-2-(1- (4-fluorophenyl) ethoxy)phenyl)-1,1- difluoromethane- sulfonamide | 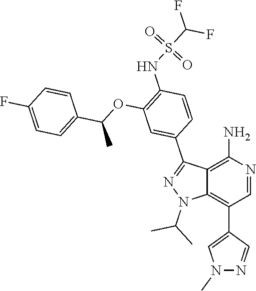 [SMILES: CC(C)n1nc(-c2ccc(NS(=O)(=O)C(F)F)c(O[C@@H](C)c3ccc(F)cc3)c2)c2c(N)ncc(-c3cnn(C)c3)c21] | 16% | LCMS (Method A): 3.19 min, m/z: 600.2 [M + H]⁺. | (400 MHz, DMSO-d₆): 7.92 (s, 1H), 7.64-7.56 (m, 4H), 7.50 (s, 1H), 7.41 (d, J = 8.0 Hz, 1H), 7.19-7.14 (m, 4H), 7.12 (s, 1H), 7.03 (s, 0.4H), 6.90 (s, 0.2H), 6.33 (s, 2H), 5.62 (q, J = 6.0 Hz, 1H), 4.59-4.53 (m, 1H), 3.92 (s, 3H), 1.59 (d, J = 5.6 Hz, 3H), 1.28 (t, J = 5.6 Hz, 6H). | 1-methyl-4- (4,4,5,5- tetramethyl- 1,3,2-dioxa- borolan- 2-yl)-1H- pyrazole |

### General Procedure A4 for the Suzuki Reaction Between Intermediate A4B1 and Boronate Esters

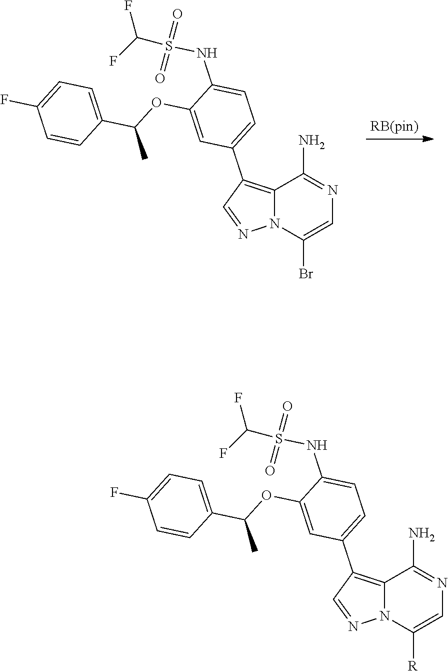

A mixture of (S)—N-(4-(4-amino-7-bromopyrazolo[1,5-a]pyrazin-3-yl)-2-(1-(4-fluorophenyl) ethoxy)phenyl)-1,1-difluoromethanesulfonamide (1 eq), boronate ester (1.1 eq), Na₂CO₃ (3 eq) and Pd(dppf)Cl₂ (0.1 eq) in degassed 1,4-dioxane/H₂O (4/1, 0.2 M) was stirred at 100° C. overnight under N₂. The mixture was diluted with water and the organics were extracted with EtOAc (3×). The combined organics were dried over Na₂SO₄ and concentrated under reduced pressure. The residue was purified by column chromatography (DCM/MeOH or PE/EtOAc) to give the title product as a yellow solid.

The following compounds were prepared following the General procedure A4

| Com- | | | | LCMS | | Boronate |
|---|---|---|---|---|---|---|
| pound | Name | Structure | Yield | data | ¹H NMR data | ester |
| 35 | (S)-N-(4-(4-amino- 7-(1-methyl-1H- pyrazol-4-yl) pyrazolo[1,5-a] pyrazin-3-yl)-2- (1-(4- fluorophenyl) ethoxy)phenyl)- 1,1- difluoromethane- sulfonamide | 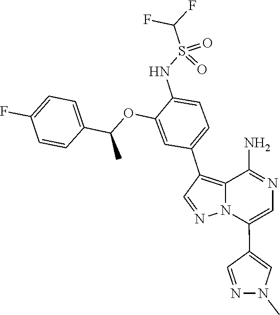 [SMILES: C[C@H](Oc1cc(-c2cnn3c(-c4cnn(C)c4)cnc(N)c23)ccc1NS(=O)(=O)C(F)F)c1ccc(F)cc1] | 31% | (Method A): 3.36 min, m/z: 558.2 [M + H]⁺. | (400 MHz, CDCl₃): 8.40 (s, 1H), 8.01 (s, 1H), 7.79 (s, 1H), 7.65-7.60 (m, 2H), 7.31 (dd, J = 8.6, 5.2 Hz, 2H), 7.10-7.03 (m, 3H), 6.86 (s, 1H), 6.37 (t, J = 53.5 Hz, 1H), 5.45-5.39 (m, 1H), 4.78 (s, 2H), 4.00 (s, 3H), 1.70 (d, J = 6.4 Hz, 3H). | 1-methyl- 4-(4,4,5,5- tetramethyl- 1,3,2-dioxa- borolan-2- yl)-1H- pyrazole |
| 36 | (S)-N-(4-(4-amino- 7-(1-(tetrahydro- 2H-pyran-4-yl)- 1H-pyrazol-4-yl) pyrazolo[1,5-a] pyrazin-3-yl)-2-(1- (4-fluorophenyl) ethoxy)phenyl)- 1,1- difluoromethane- sulfonamide | 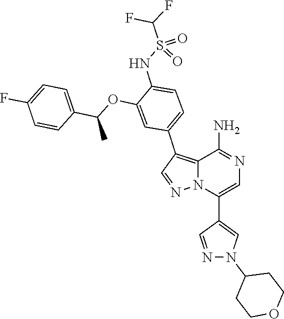 [SMILES: C[C@H](Oc1cc(-c2cnn3c(-c4cnn(C5CCOCC5)c4)cnc(N)c23)ccc1NS(=O)(=O)C(F)F)c1ccc(F)cc1] | 52% | (Method A): 3.41 min, m/z: 628.2 [M + H]⁺. | (400 MHz, DMSO-d₆): 10.47 (s, 1H), 8.64 (s, 1H), 8.24 (s, 1H), 8.01 (s, 1H), 7.82 (s, 1H), 7.64-7.54 (m, 2H), 7.36 (d, J = 7.9 Hz, 1H), 7.18 (t, J = 8.5 Hz, 2H), 7.11-6.64 (m, 3H), 5.91 (s, 2H), 5.68 (d, J = 6.2 Hz, 1H), 4.51 (td, J = 10.9, 6.0 Hz, 1H), 3.98 (d, J = 10.6 Hz, 2H), 3.50 (dd, J = 11.2, 3.2 Hz, 2H), 2.10-1.92 (m, 5H), 1.59 (d, J = 6.1 Hz, 3H). | 1- (tetrahydro- 2H-pyran- 4-yl)-4- (4,4,5,5- tetramethyl- 1,3,2-dioxa- borolan-2- yl)-1H- pyrazole |
| 37 | (S)-N-(4-(4- amino-7-(1-(2- hydroxyethyl)-1H- pyrazol-4-yl) pyrazolo[1,5-a] pyrazin-3-yl)-2-(1- (4-fluorophenyl) ethoxy)phenyl)- 1,1- difluoromethane- sulfonamide | 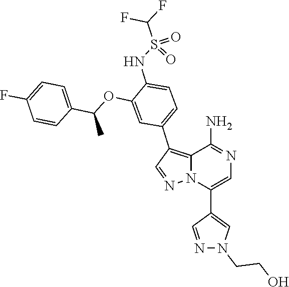 [SMILES: C[C@H](Oc1cc(-c2cnn3c(-c4cnn(CCO)c4)cnc(N)c23)ccc1NS(=O)(=O)C(F)F)c1ccc(F)cc1] | 32% | (Method A): 3.10 min, m/z: 588.2 [M + H]⁺. | (400 MHz, DMSO-d₆): 10.48 (s, 1H), 8.60 (s, 1H), 8.21 (s, 1H), 8.01 (s, 1H), 7.82 (s, 1H), 7.58 (dd, J = 8.6, 5.6 Hz, 2H), 7.35 (d, J = 8.0 Hz, 1H), 7.18 (t, J = 8.9 Hz, 2H), 7.10-6.59 (m, 4H), 5.90 (s, 2H), 5.68 (q, J = 6.2 Hz, 1H), 4.97 (t, J = 5.2 Hz, 1H), 4.23 (t, J = 5.4 Hz, 2H), 3.78 (q, J = 5.3 Hz, 2H), 1.58 (d, J = 6.2 Hz, 3H). | 2-[4- (4,4,5,5- tetramethyl- 1,3,2-dioxa- borolan-2- yl)pyrazol- 1-yl] ethanol |
| 39 | (S)-N-(4-(4- amino-7-(pyridin- 4-yl)pyrazolo[1,5- a]pyrazin-3-yl)-2- (1-(4-fluoro- phenyl)ethoxy) phenyl)-1,1- difluoromethane- sulfonamide | 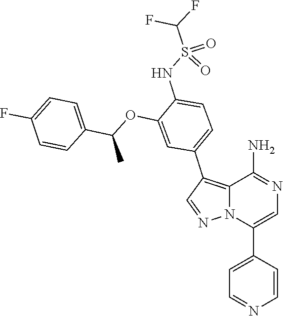 [SMILES: C[C@H](Oc1cc(-c2cnn3c(-c4ccncc4)cnc(N)c23)ccc1NS(=O)(=O)C(F)F)c1ccc(F)cc1] | 33% | (Method A): 3.15 min, m/z: 555.2 [M + H]⁺. | (400 MHz, DMSO-d₆): 10.50 (s, 1H), 8.71 (d, J = 22.1 Hz, 2H), 7.99 (d, J = 4.2 Hz, 3H), 7.77 (s, 1H), 7.58 (dd, J = 8.5, 5.6 Hz, 2H), 7.37 (d, J = 8.0 Hz, 1H), 7.23-7.14 (m, 2H), 7.10-6.86 (m, 3H), 6.42-6.22 (m, 2H), 5.68 (q, J = 6.1 Hz, 1H), 1.59 (d, J = 6.2 Hz, 3H). | 4-(4,4,5,5- tetramethyl- 1,3,2-dioxa- borolan-2- yl) pyridine |

### Synthesis of Halogenopyrazole Intermediates D

### Intermediate D1: tert-butyl (3aR,6aS)-5-(4-bromo-1H-pyrazol-1-yl)-octahydrocyclopenta [c]pyrrole-2-carboxylate

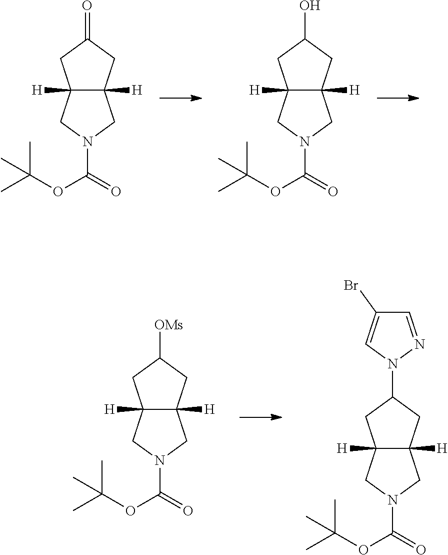 [SMILES: CC(C)(C)OC(=O)N1C[C@H]2CC(=O)C[C@H]2C1 ; CC(C)(C)OC(=O)N1C[C@H]2CC(O)C[C@H]2C1 ; CC(C)(C)OC(=O)N1C[C@H]2CC(OS(C)(=O)=O)C[C@H]2C1 ; CC(C)(C)OC(=O)N1C[C@H]2CC(n3cc(Br)cn3)C[C@H]2C1]

### Step 1: tert-butyl (3aR,6aS)-5-hydroxy-octahydrocyclopenta[c]pyrrole-2-carboxylate

To a solution of tert-butyl (3aR,6aS)-5-oxo-octahydrocyclopenta[c]pyrrole-2-carboxylate (2.3 g, 10.2 mmol) in MeOH (50 mL) was added NaBH₄ (1.92 g, 50.9 mmol) and the mixture was stirred at 60° C. overnight. The mixture was concentrated under reduced pressure and the residue was partitioned between DCM (10 mL) and H₂O (10 mL). The organic phase was removed and the aqueous was extracted with DCM (2×25 ml). The combined organics were dried over Na₂SO₄ and concentrated under reduced pressure to give the title product (2.20 g, 95%) as a yellow oil. LCMS: (Method A): 2.70 min; m/z: 228.1 [M+H]⁺

### Step 2: tert-butyl (3aR,6aS)-5-(methanesulfonyloxy)-octahydrocyclopenta[c]pyrrole-2-carboxylate

To a solution of tert-butyl (3aR,6aS)-5-hydroxy-octahydrocyclopenta[c]pyrrole-2-carboxylate (2.2 g, 9.67 mmol) and Et₃N (2.93 g, 29.0 mmol) in DCM (50 mL) was added MsCl (1.65 g, 14.5 mmol). The mixture was stirred at RT for 1 h. The organics were extracted with DCM (2×100 mL), and the combined organics were dried over Na₂SO₄ and concentrated under reduced pressure to give the title product (3.00 g, >100%) as a yellow oil. LCMS: (Method A), 3.22 min; m/z: 306.1 [M+H]⁺.

### Step 3: tert-butyl (3aR,6aS)-5-(4-bromo-1H-pyrazol-1-yl)-octahydrocyclopenta[c]pyrrole-2-carboxylate

To a mixture of tert-butyl (3aR,6aS)-5-(methanesulfonyloxy)-octahydrocyclopenta[c]pyrrole-2-carboxylate (3 g, 9.82 mmol) and Cs₂CO₃ (6.38 g, 19.6 mmol) in MeCN (60 mL) was added 4-bromo-1H-pyrazole (2.16 g, 14.7 mmol) and the mixture was stirred at 80° C. overnight. The mixture was concentrated under reduced pressure and the residue was partitioned in DCM (10 mL) and H₂O (10 mL). The organic phase was collected and the aqueous was extracted with DCM (2×25 ml). The combined organics were dried over Na₂SO₄ and concentrated under reduced pressure. The residue was purified by column chromatography on silica gel (PE/EtOAc=1/1) to give the title product (2.17 g, 62%) as a yellow solid. LCMS: (Method A), 2.19 min; m/z: 378.1, 380.1 [M+Na]⁺

The following intermediate D13 were prepared according to the synthesis of intermediate D1 with 4-iodo-1H-pyrazole and tert-butyl 4-oxo-2-(trifluoromethyl)piperidine-1-carboxylate

| Halogenopyrazole | LCMS Data | Intermediate D |
|---|---|---|
| 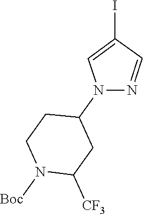 [SMILES: CC(C)(C)OC(=O)N1CCC(n2cc(I)cn2)CC1C(F)(F)F] | (Method A): 4.13 min, m/z: 446.1 [M + H]⁺ | Intermediate D13 |

### Intermediate D2: 7-(4-bromo-1H-pyrazol-1-yl)-4-methyl-4-azaspiro[2.5]octane

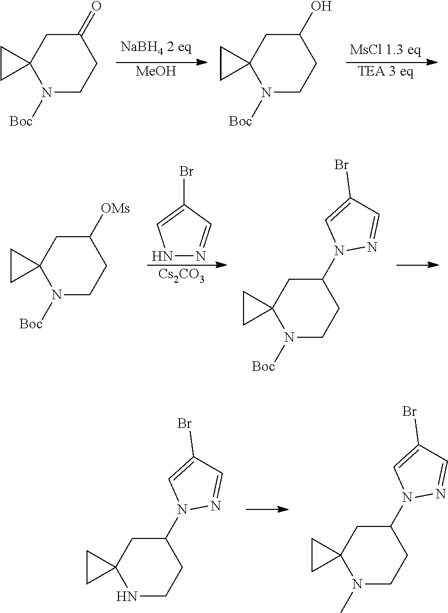

### Step 1: of tert-butyl 7-hydroxy-4-azaspiro[2.5]octane-4-carboxylate

To a solution of tert-butyl 7-oxo-4-azaspiro[2.5]octane-4-carboxylate (25 g, 110 mmol) in MeOH (400 mL) was added NaBH₄ (12.4 g, 330 mmol) and the mixture was stirred at RT overnight. The mixture was concentrated under reduced pressure and the residue was diluted with EtOAc (200 mL). The organics were washed with water (3×200 mL), dried over Na₂SO₄ and concentrated under reduced pressure to give the title product (26.0 g, >100%) as a colourless oil. Used as crude in the next step. LCMS: (Method A): 1.29 min, m/z 250.2 [M+Na]⁺

### Step 2: tert-butyl 7-(methanesulfonyloxy)-4-azaspiro[2.5]octane-4-carboxylate

To a solution of tert-butyl 7-hydroxy-4-azaspiro[2.5]octane-4-carboxylate (26 g, 114 mmol) and Et₃N (34.5 g, 342 mmol) in DCM (300 mL) was added MsCl (15.5 g, 136 mmol) and the mixture was stirred at RT for 1 h. The mixture was washed with water (3×200 mL), then the organics were dried over Na₂SO₄ and concentrated under reduced pressure to give the title product (34.0 g, 97%) as a yellow oil. LCMS: (Method A): 2.04 min, m/z 328.2 [M+Na]⁺

### Step 3: tert-butyl 7-(4-bromo-1H-pyrazol-1-yl)-4-azaspiro[2.5]octane-4-carboxylate

A mixture of tert-butyl 7-(methanesulfonyloxy)-4-azaspiro[2.5]octane-4-carboxylate (34 g, 111 mmol), 4-bromo-1H-pyrazole (19.5 g, 133 mmol) and Cs₂CO₃ (72.3 g, 222 mmol) in MeCN (350 mL) was stirred at 80° C. overnight. The reaction mixture was concentrated under reduced pressure and the residue was purified by silica gel column chromatography (PE/EA=20/1 to 1/1) to give the title product (28.0 g, 71%) as a colourless oil. LCMS: (Method A): 1.90 min, m/z 378.2, 380.2 [M+Na]⁺

### Step 4: 7-(4-bromo-1H-pyrazol-1-yl)-4-azaspiro[2.5]octane

A solution of tert-butyl 7-(4-bromo-1H-pyrazol-1-yl)-4-azaspiro[2.5]octane-4-carboxylate (28 g, 78.5 mmol) in HCl in dioxane (4 M, 60 mL) was stirred at RT for 1 h. The reaction mixture was concentrated under reduce pressure to give the title product as a HCl salt (26.0 g) as a white solid. LCMS: (Method A) 0.93 min, m/z 256.1, 258.1 [M+H]⁺

### Step 5: 7-(4-bromo-1H-pyrazol-1-yl)-4-methyl-4-azaspiro[2.5]octane

To a solution of 7-(4-bromo-1H-pyrazol-1-yl)-4-azaspiro[2.5]octane (26 g, 101 mmol) and (CH₂O)ₙ (15.2 g, 505 mmol) in DCM (300 mL) was added NaBH(OAc)₃ (31.9 g, 151 mmol) and the mixture was stirred at RT for 1 h. H₂O (100 mL) was added and the organics were separated. The organic layer was washed with water (2×100 mL), dried over Na₂SO₄ and concentrated under reduced pressure to give the title product (20.0 g, 73%) as a colorless oil. LCMS: (Method A) 0.64 min, m/z 270.2, 272.2 [M+H]⁺

The following intermediates D3-D10, D15 were prepared according to the synthesis of intermediate D2

| | | Intermediate |
|---|---|---|
| Halogenopyrazole | LCMS Data | D |
| 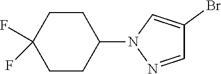 [SMILES: FC1(F)CCC(n2cc(Br)cn2)CC1] | (Method A): 1.37 min, m/z: 270.1, 272.1 [M + H]⁺ | Intermediate D3 |
| 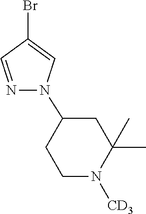 [SMILES: [2H]C([2H])([2H])N1CCC(n2cc(Br)cn2)CC1(C)C] | (Method A): 4.09 min, m/z: 275.1, 277.1 [M + H]⁺ | Intermediate D4 |
| 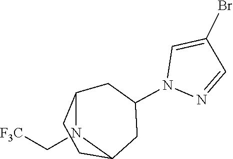 [SMILES: FC(F)(F)CN1C2CCC1CC(n1cc(Br)cn1)C2] | (Method A): 3.65 min, m/z: 338.1, 340.1 [M + H]⁺ | Intermediate D5 |
| 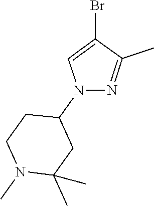 | (Method A): 1.88 min, m/z: 286.1, 288.1 [M + H]⁺ | Intermediate D6 |
| 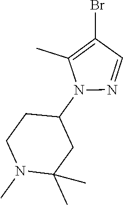 [SMILES: Cc1c(Br)cnn1C1CCN(C)C(C)(C)C1] | (Method A): 1.82 min, m/z: 286.1, 288.1 [M + H]⁺ | Intermediate D7 |
| 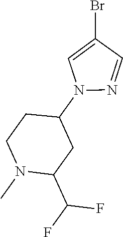 [SMILES: CN1CCC(n2cc(Br)cn2)CC1C(F)F] | (Method A): 1.26 min, m/z: 294.0, 296.0 [M + H]⁺ | Intermediate D8 |
| 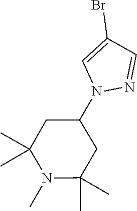 [SMILES: CN1C(C)(C)CC(n2cc(Br)cn2)CC1(C)C] | (Method A): 1.97 min, m/z: 300.1, 302.1 [M + H]⁺ | Intermediate D9 |
| 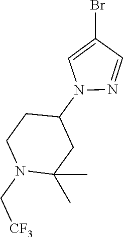 [SMILES: CC1(C)CC(n2cc(Br)cn2)CCN1CC(F)(F)F] | (Method A): 3.86 min, m/z: 340.1, 342.1 [M + H]⁺ | Intermediate D10 |
| 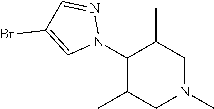 | (Method B): 1.49 min, m/z: 272.1, 274.1 [M + H]⁺ | Intermediate D15 |

### Intermediate D11 and D12: (R)-4-(4-bromo-1H-pyrazol-1-yl)-1,2,2-trimethylpiperidine and (S)-4-(4-bromo-1H-pyrazol-1-yl)-1,2,2-trimethylpiperidine

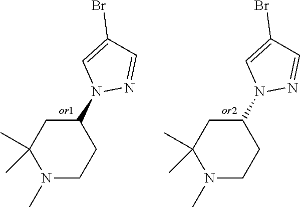

(R)-4-(4-bromo-1H-pyrazol-1-yl)-1,2,2-trimethylpiperidine and (S)-4-(4-bromo-1H-pyrazol-1-yl)-1,2,2-trimethylpiperidine were obtained by chiral SFC purification of the racemic mixture (4-(4-bromo-1H-pyrazol-1-yl)-2,2-dimethylpiperidine-1-carboxylate (obtained from tert-butyl 4-(4-(4-amino-3-(4-((difluoromethyl)sulfonamido)-3-((S)-1-(4-fluorophenyl)ethoxy)phenyl)-1-methyl-1H-pyrazolo[4,3-c]pyridin-7-yl)-1H-pyrazol-1-yl)-2,2-dimethylpiperid ine-1-carboxylate step 4 compound 13, following the synthesis of intermediate D2 step 4 and 5). Instrument SFC-150 mgm, column: YMC Cellulose-SC (20*250 mm, 5 um), temperature: 30° C., Mobile phase: C02/IPA[0.5% NH3(7 M in MeOH)]=95/5, Flow rate: 40 ml/min, Back pressure: 100 bar. Peak 1=0.89 min, Peak 2=1.09 min

The enantiomeric excess was calculated on UPCC (Waters), Column: YMC Cellulose-SC (4.6*100 mm 3 um), Temperature: 40° C., Mobile phase: CO₂/IPA [1.0% NH₃ (7 M in MeOH)]=90/10, Flow rate: 3 mL/min. Both enantiomers have an enantiomeric excess >98% ee.

### Intermediate D13 and D14: (R)-7-(4-bromo-1H-pyrazol-1-yl)-4-methyl-4-azaspiro[2.5]octane and (S)-7-(4-bromo-1H-pyrazol-1-yl)-4-methyl-4-azaspiro[2.5]octane

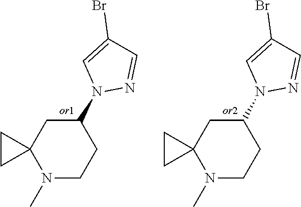

(R)-7-(4-bromo-1H-pyrazol-1-yl)-4-methyl-4-azaspiro[2.5]octane and (S)-7-(4-bromo-1H-pyrazol-1-yl)-4-methyl-4-azaspiro[2.5]octane were obtained by chiral HPLC purification of the racemic 7-(4-bromo-1H-pyrazol-1-yl)-4-methyl-4-azaspiro[2.5]octane (intermediate D2). Column: CHIRALCEL® AD-H; Column size: 21.2×250 mm, 5 μm; mobile phase: n-Hexane/Ethanol/Dietylamine=70/30/0.1 (v/v/v); flow rate: 20 mL/min; temperature: 35° C.

The enantiomeric excess was calculated: CHIRALCEL® AD-H, column size: 0.46 cm I.D.×25 cm L×5 μm, injection: 5 μL, mobile phase: n-Hexane/Ethanol/Dietylamine=70/30/0.1 (v/v/v), flow rate: 1.0 mL/min, sample solution: 1.1 mg/mL in MeOH/EtOH=1/1 (v/v). Peak 1=4.72 min, Peak 2=9.64 min. Both enantiomers have an enantiomeric excess >99% ee.

### General Procedure B for the Sonogashira Reaction Between Intermediate A1B1 and Alkynes

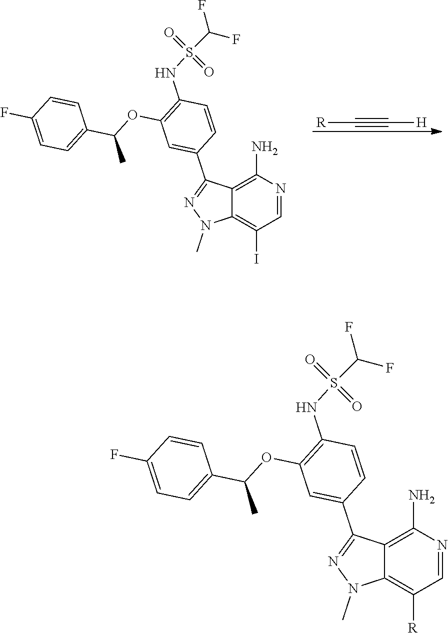

To a solution of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (1 eq.), Cul (1 eq.), Et₃N (3 eq) and the alkyne (2 eq) in degassed DMF (0.2 M) was added Pd(dppf)Cl₂ (0.1 eq) and the mixture was stirred at 130° C. with μW irradiation for 1 h, or until reaction completion as checked by TLC. The mixture was concentrated under reduced pressure and the residue was purified by column chromatography (DCM/MeOH) or prep-TLC to give the title compound as a yellow solid.

The following compounds were prepared following the General procedure B.

TABLE 6
Compounds prepared following the General procedure B

| Com- | | | | LCMS | | Alkyne |
|---|---|---|---|---|---|---|
| pound | Name | Structure | Yield | data | ¹H NMR data | SM |
| 56 | (S)-N-(4-(4-amino- 1-methyl-7-(prop-1- yn-1-yl)-1H- pyrazolo[4,3-c] pyridin-3-yl)-2-(1- (4-fluorophenyl) ethoxy)phenyl)-1,1- difluoromethane- sulfonamide | 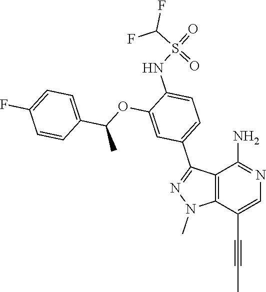 [SMILES: CC#Cc1cnc(N)c2c(-c3ccc(NS(=O)(=O)C(F)F)c(O[C@@H](C)c4ccc(F)cc4)c3)nn(C)c12] | 35% | (Method A): 3.37 min, m/z: 530.2 [M + H]⁺. | (400 MHz, DMSO-d₆): 7.85 (s, 1H), 7.64-7.55 (m, 1H), 7.40 (d, J = 8.8 Hz, 1H), 7.18-7.12 (m, 4H), 7.01 (t, J = 52.8 Hz, 1H), 5.99 (s, 2H), 5.66 (q, J = 6.4 Hz, 1H), 4.22 (s, 3H), 2.12 (s, 3H), 1.58 (d, J = 6.4 Hz, 3H) | Propyne (1M in DMF) |
| 58 | (S)-N-(4-(4-amino- 1-methyl-7- ((tetrahydro-2H- pyran-4-yl)ethynyl)- 1H-pyrazolo[4,3-c] pyridin-3-yl)-2-(1-(4- fluorophenyl) ethoxy)phenyl)-1,1- difluoromethane- sulfonamide | 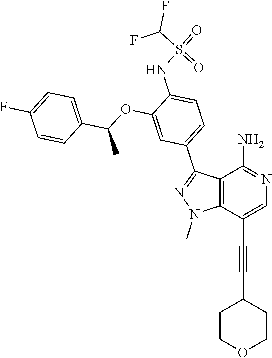 [SMILES: C[C@H](Oc1cc(-c2nn(C)c3c(C#CC4CCOCC4)cnc(N)c23)ccc1NS(=O)(=O)C(F)F)c1ccc(F)cc1] | 62% | (Method A): 3.29 min, m/z: 600.2 [M + H]⁺. | (400 MHz, DMSO-d₆): 7.66-7.16 (m, 8H), 6.09-5.74 (m, 3H), 4.23 (s, 3H), 3.84-3.80 (m, 2H), 3.48-3.42 (m, 3H), 3.32-2.96 (m, 1H), 1.89-1.85 (m, 3H) | 4- ethynyl- tetrahydro- 2H- pyran |
| 128 | (S)-N-(4-(4-amino- 1-methyl-7-((1-(2,2,2- trifluoroethyl) piperidin-4-yl) ethynyl)-1H- pyrazolo[4,3-c] pyridin-3-yl)-2-(1-(4- fluorophenyl) ethoxy)phenyl)-1,1- difluoromethane- sulfonamide | 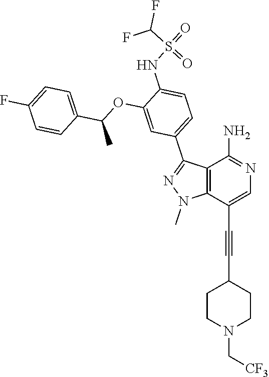 [SMILES: C[C@H](Oc1cc(-c2nn(C)c3c(C#CC4CCN(CC(F)(F)F)CC4)cnc(N)c23)ccc1NS(=O)(=O)C(F)F)c1ccc(F)cc1] | 11% | (Method A): 3.54 min, m/z: 681.1 [M + H]⁺. | (400 MHz, DMSO-d₆) 10.56 (s, 1H), 7.82 (s, 1H), 8.06 (s, 1H), 7.56 (q, J = 5.2 Hz, 2H), 7.40 (d, J = 8 Hz, 1H), 7.18-7.11 (m, 4H), 7.01 (t, J = 52 Hz, 1H), 6.02 (s, 2H), 5.65 (q, J = 6.8 Hz, 1H), 4.23 (s, 3H), 3.17 (q, J = 10.7 Hz, 2H), 2.89-2.84 (m, 2H), 2.75-2.71 (m, 1H), 2.53 (s, 2H), 1.92-1.88 (m, 2H), 1.71-1.62 (m, 2H), 1.59 (d, J = 4 Hz, 3H). | 4-ethynyl- 1-(2,2,2- trifluoro- ethyl) piperidine |

### Compounds 79 and 80

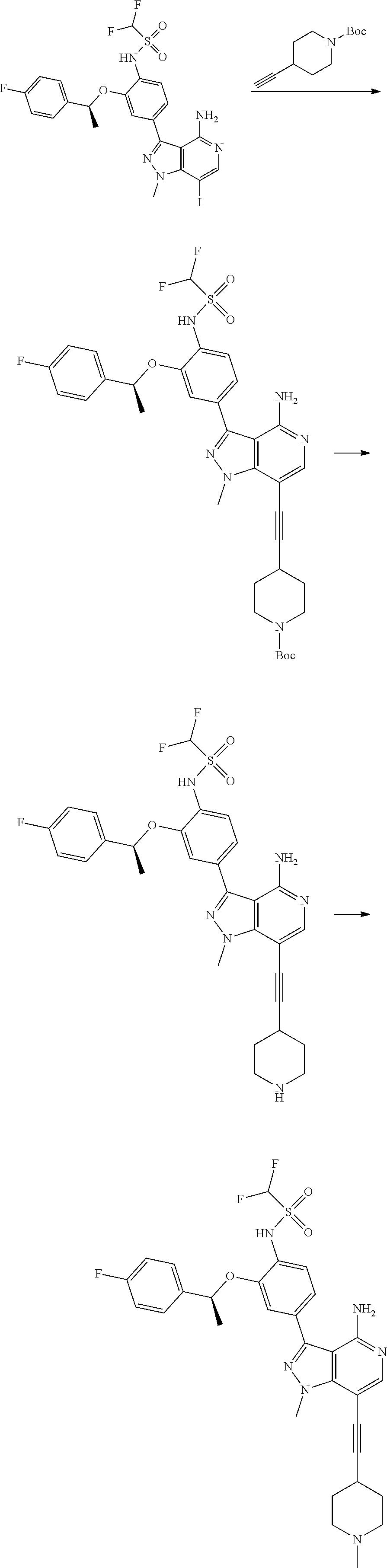 [SMILES: C[C@H](Oc1cc(-c2nn(C)c3c(I)cnc(N)c23)ccc1NS(=O)(=O)C(F)F)c1ccc(F)cc1 ; C#CC1CCN(C(=O)OC(C)(C)C)CC1 ; C[C@H](Oc1cc(-c2nn(C)c3c(C#CC4CCN(C(=O)OC(C)(C)C)CC4)cnc(N)c23)ccc1NS(=O)(=O)C(F)F)c1ccc(F)cc1 ; C[C@H](Oc1cc(-c2nn(C)c3c(C#CC4CCNCC4)cnc(N)c23)ccc1NS(=O)(=O)C(F)F)c1ccc(F)cc1 ; C[C@H](Oc1cc(-c2nn(C)c3c(C#CC4CCN(C)CC4)cnc(N)c23)ccc1NS(=O)(=O)C(F)F)c1ccc(F)cc1]

### Step 1: tert-butyl 4-(2-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}ethynyl)piperidine-1-carboxylate

Following general procedure B with tert-butyl 4-ethynylpiperidine-1-carboxylate, the title compound (88%) was obtained as a yellow solid.

### Step 2: (S)—N-(4-(4-amino-1-methyl-7-(piperidin-4-ylethynyl)-1H-pyrazolo[4,3-c]pyridin-3-yl)-2-(1-(4-fluorophenyl)ethoxy)phenyl)-1,1-difluoromethanesulfonamide (Compound 79)

To a solution of tert-butyl 4-(2-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}ethynyl)piperidine-1-carboxylate (300 mg, 429 μmol) in HCO₂H (2 mL) was stirred at RT for 1 h. The reaction mixture was concentrated under reduced pressure and the residue was adjusted to pH=8 with sat. aq. Na₂CO₃. The residue was diluted with water (5 mL) and the organics were extracted with EtOAc (3×5 mL). The combined organics were washed with brine, dried over Na₂SO₄ and concentrated under reduced pressure. The residue was purified by prep-TLC (DCM/MeOH: 95/5) to give the title product (140 mg, 54%) as a white solid. LCMS (Method A): 2.83 min, m/z 599.1 [M+H]⁺. ¹H NMR (400 MHz, DMSO-d₆): 7.80 (s, 1H), 7.64-7.53 (m, 1H), 7.50 (q, J=4 Hz, 2H), 7.34 (d, J=8 Hz, 1H), 7.12 (t, J=8 Hz, 1H), 6.90 (d, J=8 Hz, 1H), 7.18-7.10 (m, 1H), 6.84 (s, 1H), 6.33 (t, J=52 Hz, 1H), 6.00 (s, 2H), 5.63 (q, J=8 Hz, 1H), 4.15 (s, 3H), 3.23 (s, 2H), 3.00 (s, 3H), 2.04 (s, 2H), 1.76 (s, 2H), 1.50 (d, J=6.2 Hz, 3H)

### Step 3: (S)—N-(4-(4-amino-1-methyl-7-((1-methylpiperidin-4-yl)ethynyl)-1H-pyrazolo[4,3-c]pyridin-3-yl)-2-(1-(4-fluorophenyl)ethoxy)phenyl)-1,1-difluoromethanesulfonamide (Compound 80)

To a solution of N-(4-{4-amino-1-methyl-7-[2-(piperidin-4-yl)ethynyl]-1H-pyrazolo[4,3-c]pyridin-3-yl}-2-[(1S)-1-(4-fluorophenyl)ethoxy]phenyl)-1,1-difluoromethanesulfonamide (120 mg, 0.2 mmol) in DCM (10 mL) were added paraformaldehyde (11.9 mg, 0.4 mmol) and NaBH(OAc)₃ (127 mg, 0.6 mmol), and the reaction mixture was stirred at RT overnight. The mixture was concentrated under reduced pressure and the residue was purified by prep-TLC (MeOH/DCM: 1/20) to give the title compound (40 mg, 32%) as a yellow solid. LCMS (Method A): 2.80 min, m/z 613.3 [M+H]⁺. ¹H NMR (400 MHz, DMSO-d₆): 10.96 (s, 1H), 7.88 (s, 1H), 7.57 (q, J=4 Hz, 2H), 7.40 (d, J=8 Hz, 1H), 7.18-7.10 (m, 4H), 7.13 (t, J=52 Hz, 1H), 6.11 (s, 2H), 5.65 (q, J=4 Hz, 1H), 4.22 (s, 3H), 3.16 (s, 2H), 2.89 (s, 1H), 2.70 (s, 3H), 2.18-1.98 (m, 4H), 1.57 (d, J=6.2 Hz, 3H), 1.23 (s, 2H).

### Compound 91: (S)—N-(4-(4-amino-1-methyl-7-(5-methyl-4,5,6,7-tetrahydropyrazolo[1,5-a]pyrazin-3-yl)-1H-pyrazolo[4,3-c]pyridin-3-yl)-2-(1-(4-fluorophenyl)ethoxy)phenyl)-1,1-difluoromethanesulfonamide

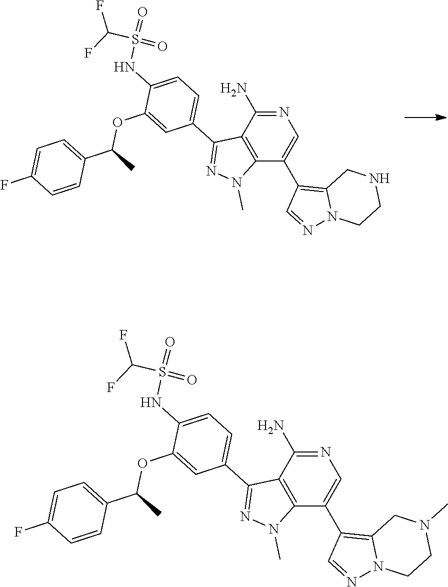 [SMILES: C[C@H](Oc1cc(-c2nn(C)c3c(-c4cnn5c4CNCC5)cnc(N)c23)ccc1NS(=O)(=O)C(F)F)c1ccc(F)cc1 ; C[C@H](Oc1cc(-c2nn(C)c3c(-c4cnn5c4CN(C)CC5)cnc(N)c23)ccc1NS(=O)(=O)C(F)F)c1ccc(F)cc1]

### Step 1: (S)—N-(4-(4-amino-1-methyl-7-(5-methyl-4,5,6,7-tetrahydropyrazolo[1,5-a]pyrazin-3-yl)-1H-pyrazolo[4,3-c]pyridin-3-yl)-2-(1-(4-fluorophenyl)ethoxy)phenyl)-1,1-difluoromethanesulfonamide

To a solution of (S)—N-(4-(4-amino-1-methyl-7-(4,5,6,7-tetrahydropyrazolo[1,5-a]pyrazin-3-yl)-1H-pyrazolo[4,3-c]pyridin-3-yl)-2-(1-(4-fluorophenyl)ethoxy)phenyl)-1,1-difluoromethanesulfonamide (Compound 90, 34 mg, 55.4 μmol) in DCM (5 mL) were added paraformaldehyde (5.37 mg, 66.4 μmol) and NaBH(OAc)₃ (58.7 mg, 277 μmol). The reaction mixture was stirred at RT for 45 min before sat. aq. NaHCO₃ (10 mL) was added, and the aqueous layer was extracted with EtOAc (3×10 mL). The combined organics were concentrated under reduced pressure and the residue was purified by silica gel column chromatography (DCM:MeOH; 10:1) to give the title compound (10 mg, 29%) as a yellow solid. LCMS (Method B): 2.85 min; m/z: 627.2 [M+H]⁺. ¹H NMR (400 MHz, DMSO-d₆): 7.60 (s, 1H), 7.53 (d, J=8.1 Hz, 1H), 7.48 (dd, J=8.4, 5.4 Hz, 3H), 7.16 (d, J=8.1 Hz, 1H), 7.10-7.02 (m, 3H), 5.56 (q, J=6.2 Hz, 1H), 4.29 (t, J=5.5 Hz, 2H), 3.70 (s, 3H), 3.57 (s, 2H), 3.04 (t, J=5.4 Hz, 2H), 2.48 (s, 3H), 1.69 (d, J=6.3 Hz, 3H).

The following compounds were prepared similarly to compound 91

| Com- | | | | LCMS | | |
|---|---|---|---|---|---|---|
| pound | Name | Structure | Yield | data | ¹H NMR data | SM |
| 99 | (S)-N-(4-(4-amino-1- methyl-7-(1-(2-methyl- 2-azaspiro[3.3]heptan- 6-yl)-1H-pyrazol-4- yl)-1H-pyrazolo[4,3- c]pyridin-3-yl)-2-(1-(4- fluorophenyl)eth- oxy)phenyl)-1,1- difluoromethane- sulfonamide | 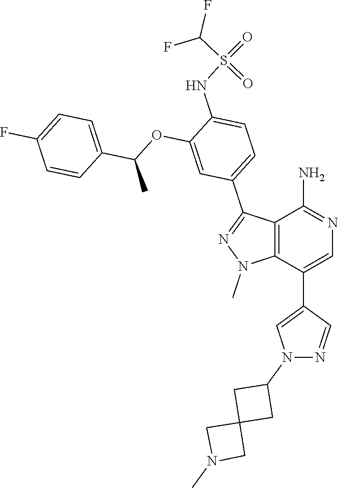 [SMILES: C[C@H](Oc1cc(-c2nn(C)c3c(-c4cnn(C5CC6(C5)CN(C)C6)c4)cnc(N)c23)ccc1NS(=O)(=O)C(F)F)c1ccc(F)cc1] | 19% | (Method A): 2.78 min, m/z: 667.2 [M + H]⁺. | (400 MHz, MeOD-d₄): 7.83 (s, 1H), 7.66 (s, 1H), 7.49 (t, J = 4.5 Hz, 4H), 7.14 (dd, J = 8.2, 1.8 Hz, 1H), 7.08-7.02 (m, 3H), 5.55 (q, J = 6.3 Hz, 1H), 4.20 (s, 2H), 4.14 (s, 2H), 3.72 (s, 3H), 2.95-2.85 (m, 4H), 2.84 (s, 4H), 1.69 (d, J = 6.4 Hz, 3H). | 78 |
| 148 | N-(4-(4-amino-1-methyl- 7-(1-(1-methyl-2- (trifluoromethyl)piperidin- 4-yl)-1H-pyrazol-4- yl)-1H-pyrazolo[4,3- c]pyridin-3-yl)-2-((S)-1- (4-fluorophenyl)eth- oxy)phenyl)-1,1- difluoromethane- sulfonamide | 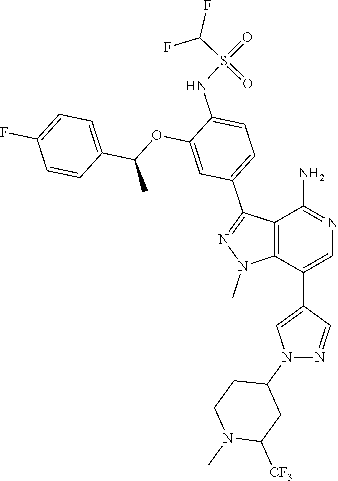 [SMILES: C[C@H](Oc1cc(-c2nn(C)c3c(-c4cnn(C5CCN(C)C(C(F)(F)F)C5)c4)cnc(N)c23)ccc1NS(=O)(=O)C(F)F)c1ccc(F)cc1] | 23% | (Method A): 2.92 min, m/z: 723.2 [M + H]⁺ | (400 MHz, MeOD-d₄): 7.83 (s, 1H), 7.54 (s, 1H), 7.42 (t, J = 4.1 Hz, 2H), 7.41-7.35 (m, 2H), 7.06 (dd, J = 8.0, 1.8 Hz, 1H), 7.02-6.92 (m, 3H), 6.58 (t, J = 53.3 Hz, 1H), 5.46 (q, J = 6.4 Hz, 1H), 4.55-4.44 (m, 1H), 3.63 (s, 3H), 3.55-3.46 (m, 1H), 2.96 (d, J = 11.7 Hz, 1H), 2.76 (d, J = 12.8 Hz, 1H), 2.54 (d, J = 2.2 Hz, 3H), 2.38-2.29 (m, 1H), 2.22 (d, J = 14.1 Hz, 1H), 2.12 (td, J = 13.8, 12.8, 5.0 Hz, 1H), 2.00 (dd, J = 12.7, 4.0 Hz, 1H), 1.59 (d, J = 6.4 Hz, 3H). | 149 |
| 164 | N-(4-(4-amino-1-methyl- 7-(1-((3aR,6aS)-2- methyloctahydrocyclo- penta[c]pyrrol-5-yl)- 1H-pyrazol-4-yl)-1H- pyrazolo[4,3-c]pyridin- 3-yl)-2-((S)-1-(4- fluorophenyl)eth- oxy)phenyl)-1,1- difluoromethane- sulfonamide | 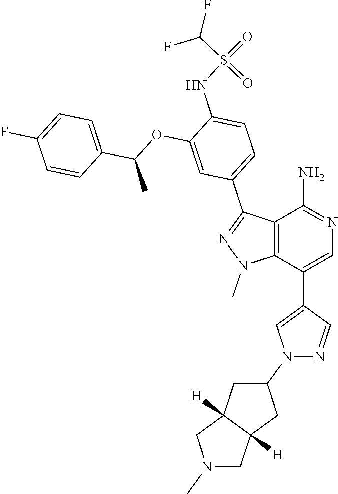 [SMILES: C[C@H](Oc1cc(-c2nn(C)c3c(-c4cnn(C5C[C@@H]6CN(C)C[C@@H]6C5)c4)cnc(N)c23)ccc1NS(=O)(=O)C(F)F)c1ccc(F)cc1] | 31% | (Method A): 2.29 min, m/z: 681.2 [M + H]⁺ | (400 MHz, DMSO-d₆): 10.69 (s, 1H), 7.96 (s, 1H), 7.60 (s, 1H), 7.56-7.53 (m, 3H), 7.38 (d, J = 8 Hz), 7.17-7.01 (m, 4H), 6.78 (t, J = 52 Hz, 1H), 5.68 (s, 2H), 5.65 (q, J = 6.8 Hz, 1H), 5.05-4.98 (m, 1H), 3.68 (s, 3H), 3.24 (s, 2 H), 2.96 (s, 4H), 2.66 (s, 3H), 2.28- 2.08 (m, 4H), 1.55 (d, J = 4 Hz, 3H) | 163 |

### Compound 54 and Compound 55

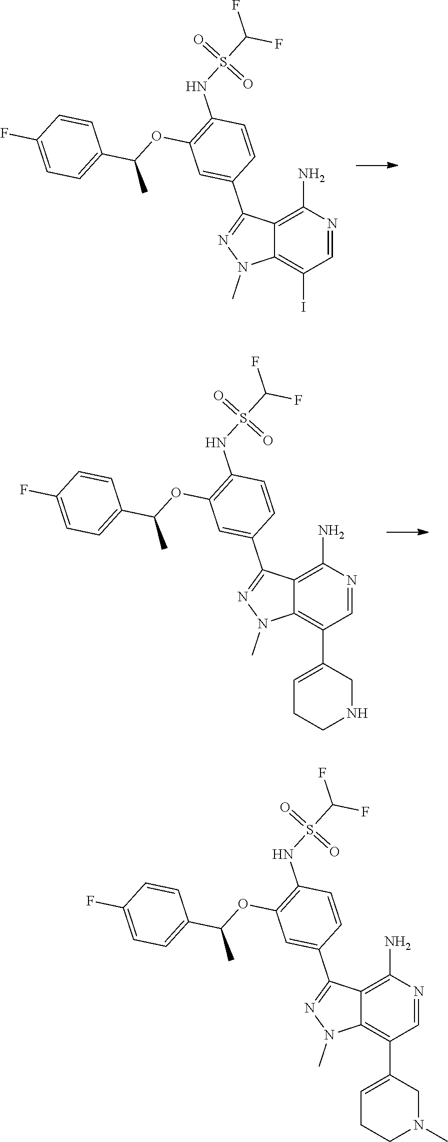

### Step 1: (S)—N-(4-(4-amino-1-methyl-7-(1,2,5,6-tetrahydropyridin-3-yl)-1H-pyrazolo[4,3-c]pyridin-3-yl)-2-(1-(4-fluorophenyl)ethoxy)phenyl)-1,1-difluoromethanesulfonamide (Compound 54)

To a solution of tert-butyl 5-(4,4,5,5-tetramethyl-1,3,2-dioxaborolan-2-yl)-3,6-dihydropyridine-1(2H)-carboxylate (1 g, 3.23 mmol) in 1,4-dioxane (15 mL) was added a 2 M HCl in dioxane (10 mL) and the mixture was stirred at RT for 10 min. The solution was concentrated under reduced pressure to give a white solid. The solids were dissolved in degassed 1,4-dioxane/H₂O (4/1, 10 mL) and (S)—N-(4-(4-amino-7-iodo-1-methyl-1H-pyrazolo[4,3-c]pyridin-3-yl)-2-(1-(4-fluorophenyl)ethoxy)phenyl)-1,1-difluoromethanesulfonamide (700 mg, 1.13 mmol), Na₂CO₃ (358 mg, 3.38 mmol) and Pd(dppf)Cl₂ (92.2 mg, 113 μmol) were added. The solution was then stirred at 100° C. for 12 h. The reaction mixture was concentrated under reduced pressure, and the residue was poured into water (20 mL) and extracted with EtOAc (2×20 mL). The combined organics were dried over Na₂SO₄ and concentrated under reduced pressure. The residue was purified by prep-TLC (DCM/MeOH=10/1) to give the title product (300 mg, 46%) as a brown solid. LCMS (Method A): 2.63 min, m/z: 573.2 [M+H]⁺. ¹H NMR (400 MHz, DMSO-d₆): 10.85 (s, 1H), 7.54 (dd, J=8.7, 5.6 Hz, 2H), 7.48 (s, 1H), 7.37 (d, J=8.1 Hz, 1H), 7.15 (t, J=8.9 Hz, 2H), 7.06-6.98 (m, 2H), 5.87 (s, 1H), 5.64 (q, J=6.3 Hz, 1H), 3.97 (s, 3H), 3.52 (s, 2H), 2.99 (s, 2H), 2.61 (s, 3H), 2.47 (s, 2H), 1.55 (d, J=6.3 Hz, 3H).

### Step 2: (S)—N-(4-(4-amino-1-methyl-7-(1-methyl-1,2,5,6-tetrahydropyridin-3-yl)-1H-pyrazolo[4,3-c]pyridin-3-yl)-2-(1-(4-fluorophenyl)ethoxy)phenyl)-1,1-difluoromethanesulfonamide (Compound 55)

A mixture of (S)—N-(4-(4-amino-1-methyl-7-(1,2,5,6-tetrahydropyridin-3-yl)-1H-pyrazolo[4,3-c]pyridin-3-yl)-2-(1-(4-fluorophenyl)ethoxy)phenyl)-1,1-difluoromethanesulfonamide (20 mg, 34.9 μmol) and paraformaldehyde (20.8 mg, 348 μmol) in MeOH (3 mL) was stirred at RT for 12 h. NaBH₃(CN) (8.73 mg, 139 μmol) was added and the mixture was stirred at RT for 20 min. The reaction mixture was poured into water (40 mL) and the organics were extracted with EtOAc (2×40 mL). The combined organics were dried over Na₂SO₄ and concentrated under reduced pressure. The residue was purified by column chromatography (DCM/MeOH=15/1) to give the title product (10.0 mg, 49%) as a brown solid. LCMS (Method A): 3.73 min, m/z: 611.2 [M+Na]⁺. ¹H NMR (400 MHz, DMSO-d₆): 10.85 (s, 1H), 7.54 (dd, J=8.7, 5.6 Hz, 2H), 7.48 (s, 1H), 7.37 (d, J=8.1 Hz, 1H), 7.15 (t, J=8.9 Hz, 2H), 7.06-6.98 (m, 2H), 5.87 (s, 1H), 5.64 (q, J=6.3 Hz, 1H), 3.97 (s, 3H), 3.52 (s, 2H), 2.99 (s, 2H), 2.61 (s, 3H), 2.47 (s, 2H), 1.55 (d, J=6.3 Hz, 3H).

### Compound 42: (S)—N-(4-(4-amino-1-methyl-7-(1-methylpiperidin-4-yl)-1H-pyrazolo[4,3-c]pyridin-3-yl)-2-(1-(4-fluorophenyl)ethoxy)phenyl)-1,1-difluoromethanesulfonamide

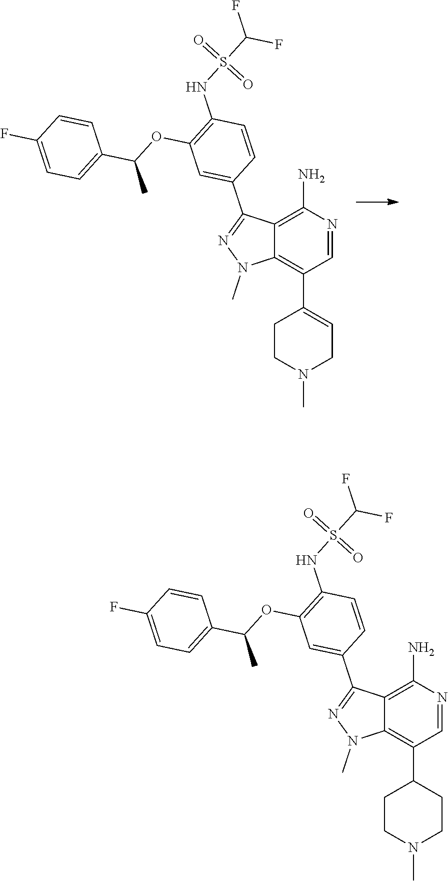 [SMILES: C[C@H](Oc1cc(-c2nn(C)c3c(C4=CCN(C)CC4)cnc(N)c23)ccc1NS(=O)(=O)C(F)F)c1ccc(F)cc1 ; C[C@H](Oc1cc(-c2nn(C)c3c(C4CCN(C)CC4)cnc(N)c23)ccc1NS(=O)(=O)C(F)F)c1ccc(F)cc1]

### Step 1: (S)—N-(4-(4-amino-1-methyl-7-(1-methylpiperidin-4-yl)-1H-pyrazolo[4,3-c]pyridin-3-yl)-2-(1-(4-fluorophenyl)ethoxy)phenyl)-1,1-difluoromethanesulfonamide

A mixture of (S)—N-(4-(4-amino-1-methyl-7-(1-methyl-1,2,3,6-tetrahydropyridin-4-yl)-1H-pyrazolo[4,3-c]pyridin-3-yl)-2-(1-(4-fluorophenyl)ethoxy)phenyl)-1,1-difluoromethanesulfonamide (90 mg, 153 μmol), NaBH₄ (11.5 mg, 306 μmol) and AcOH (27.5 mg, 459 μmol) in THE (10 mL) was stirred at 45° C. overnight. HCl (1 M, 5 mL) was added, and the reaction mixture was stirred for 5 min. The reaction mixture was adjusted to pH=8 with sat. aq. Na₂CO₃ and the organics were extracted with EtOAc (2×30 mL). The combined organic phases were dried over Na₂SO₄ and concentrated. The residue was purified by prep-TLC (DCM/MeOH=15/1) to give the title product (7 mg, 7%) as a white solid. LCMS (Method A): 3.30 min, m/z 589.2 [M+H]⁺. ¹H NMR (400 MHz, CDCl₃): 7.86 (s, 1H), 7.69 (d, J=8.2 Hz, 1H), 7.34-7.29 (m, 2H), 7.15 (d, J=8.2 Hz, 1H), 7.08 (t, J=8.6 Hz, 2H), 6.92 (s, 1H), 6.35 (t, J=53.6 Hz, 1H), 5.72 (s, 2H), 5.43 (q, J=6.1 Hz, 1H), 4.21 (s, 3H), 3.26 (d, J=8.1 Hz, 2H), 2.99 (s, 2H), 2.74 (s, 3H), 2.22 (d, J=15.3 Hz, 1H), 2.00 (s, 2H), 1.82 (s, 2H), 1.70 (d, J=6.4 Hz, 3H).

### Compound 40: (S)—N-(4-(4-amino-1-methyl-7-(pyrimidin-4-yl)-1H-pyrazolo[4,3-c]pyridin-3-yl)-2-(1-(4-fluorophenyl)ethoxy)phenyl)-1,1-difluoromethanesulfonamide

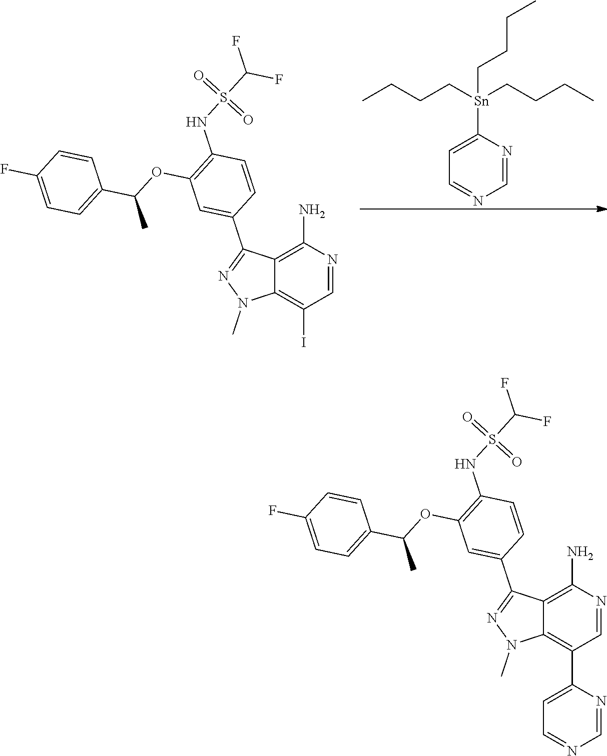 [SMILES: C[C@H](Oc1cc(-c2nn(C)c3c(I)cnc(N)c23)ccc1NS(=O)(=O)C(F)F)c1ccc(F)cc1 ; CCC[CH2][Sn]([CH2]CCC)([CH2]CCC)[c]1ccncn1 ; C[C@H](Oc1cc(-c2nn(C)c3c(-c4ccncn4)cnc(N)c23)ccc1NS(=O)(=O)C(F)F)c1ccc(F)cc1]

### Step 1: (S)—N-(4-(4-amino-1-methyl-7-(pyrimidin-4-yl)-1H-pyrazolo[4,3-c]pyridin-3-yl)-2-(1-(4-fluorophenyl)ethoxy)phenyl)-1,1-difluoromethanesulfonamide

To a solution of (S)—N-(4-(4-amino-7-iodo-1-methyl-1H-pyrazolo[4,3-c]pyridin-3-yl)-2-(1-(4-fluorophenyl)ethoxy)phenyl)-1,1-difluoromethanesulfonamide (70 mg, 113 μmol) in degassed MePh (2 mL) were added Pd(PPh₃)₄ (13.0 mg, 11.3 μmol), KF (32.8 mg, 565 μmol) and 4-(tributylstannyl)pyrimidine (41.7 mg, 113 μmol), and the mixture was stirred at 130° C. with μW irradiation for 2 h. The mixture was concentrated under reduced pressure and the residue was purified by Prep TLC (DCM/MeOH=40/1) to give the title product (7.0 mg, 11%) as a yellow solid. LCMS (method C): 3.30 min, m/z 570.1 [M+H]⁺. ¹H NMR (400 MHz, CDCl₃): 9.30 (s, 1H), 8.81 (d, J=5.2 Hz, 1H), 7.92 (s, 1H), 7.68 (d, J=8.4 Hz, 1H), 7.59 (d, J=4.4 Hz, 1H), 7.32 (q, J=4.4 Hz, 2H), 7.21 (dd, J=8.4, 1.6 Hz, 1H), 7.07-7.02 (m, 3H), 6.35 (t, J=53.6 Hz, 1H), 5.47 (q, J=6.4 Hz, 1H), 5.13 (s, 2H), 3.95 (s, 3H), 1.69 (d, J=6.4 Hz, 3H).

### Compound 109: (S)—N-(4-(7-(1-(1-acetylpiperidin-4-yl)-1H-pyrazol-4-yl)-4-amino-1-methyl-1H-pyrazolo[4,3-c]pyridin-3-yl)-2-(1-(4-fluorophenyl)ethoxy)phenyl)-1,1-difluoromethanesulfonamide

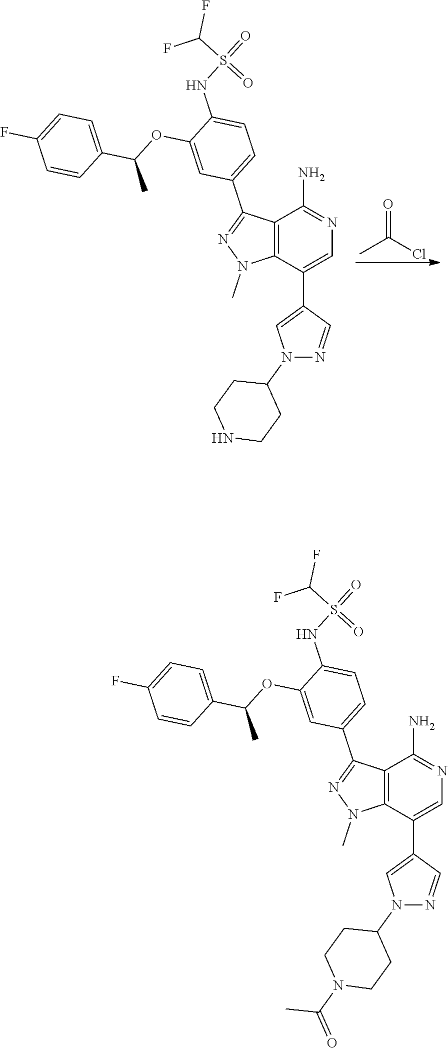

### Step 1: (S)—N-(4-(7-(1-(1-acetylpiperidin-4-yl)-1H-pyrazol-4-yl)-4-amino-1-methyl-1H-pyrazolo[4,3-c]pyridin-3-yl)-2-(1-(4-fluorophenyl)ethoxy)phenyl)-1,1-difluoromethanesulfonamide

To a solution of compound 59 (150 mg, 234 μmol) and Et₃N (70.9 mg, 702 μmol) in DCM (10 mL) was added AcCl (18.3 mg, 234 μmol). The reaction mixture was stirred at RT for 10 min. The mixture was concentrated under reduced pressure and the residue was purified by prep-TLC (DCM:MeOH; 20:1) to give the title product (80.0 mg, 50%) as a yellow solid. LCMS (Method A) 2.88 min; m/z 683.2 [M+H]⁺. ¹H NMR (400 MHz, DMSO-d₆): 7.99 (s, 1H), 7.61 (s, 1H), 7.59-7.55 (m, 3H), 7.43-7.41 (d, J=8 Hz, 1H), 7.18-7.14 (m, 4H), 6.99 (t, J=52 Hz, 1H), 5.82 (s, 2H), 5.66 (q, J=4 Hz, 1H), 4.55-4.44 (m, 2H), 3.70 (s, 3H), 2.80-2.74 (m, 1H), 2.15-2.11 (m, 2H), 2.05 (s, 3H), 1.99-1.80 (m, 2H), 1.60 (d, J=8 Hz, 3H).

### Compound 89: (S)—N-(4-(4-amino-7-(1-(1-(aminomethyl)cyclopropyl)-1H-pyrazol-4-yl)-1-methyl-1H-pyrazolo[4,3-c]pyridin-3-yl)-2-(1-(4-fluorophenyl)ethoxy)phenyl)-1,1-difluoromethanesulfonamide

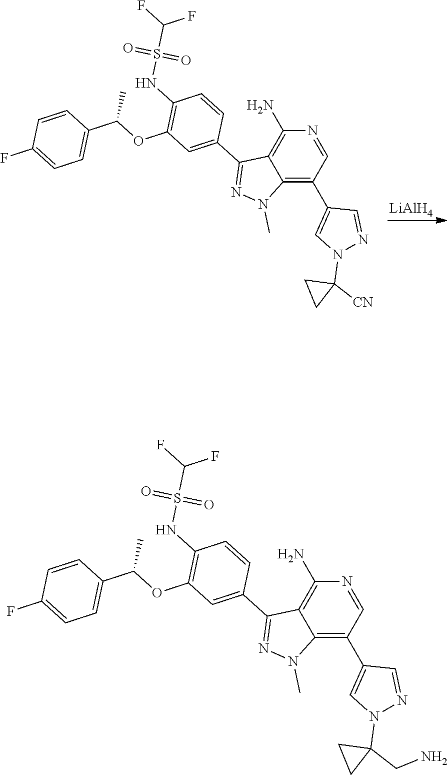

### Step 1: (S)—N-(4-(4-amino-7-(1-(1-(aminomethyl)cyclopropyl)-1H-pyrazol-4-yl)-1-methyl-1H-pyrazolo[4,3-c]pyridin-3-yl)-2-(1-(4-fluorophenyl)ethoxy)phenyl)-1,1-difluoromethanesulfonamide

To a 0° C. solution of N-(4-{4-amino-7-[1-(1-cyanocyclopropyl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide 96 (30 mg, 48.1 μmol) in dry THE (2 mL) was added LiAlH₄ (3.65 mg, 96.2 μmol). The reaction mixture was stirred at RT for 1 h under N₂, then concentrated under reduced pressure. The residue was purified by prep-TLC (DCM:MeOH; 98:2) to give the title product (15.0 mg, 50%) as yellow solid. LCMS (Method E): 0.34 min, m/z: 627.3 [M+H]⁺. ¹H NMR (400 MHz, DMSO-d₆): 7.99 (s,1H), 7.71 (s, 1H), 7.53-7.49 (m, 3H), 7.36-7.34 (m, 1H), 7.13 (t, J=8.4 Hz, 2H), 6.95-6.87 (m, 2H), 6.40 (t, J=14.4 Hz, 1H), 5.65 (brs, 3H), 3.72 (s, 3H), 3.27 (s, 2H), 1.52-1.23 (m, 7H).

### Compound 141: N-{4-[4-amino-1-methyl-7-(morpholin-4-yl)-1H-pyrazolo[4,3-c]pyridin-3-yl]-2-[(1S)-1-(4-fluorophenyl)ethoxy]phenyl}-1,1-difluoromethanesulfonamide

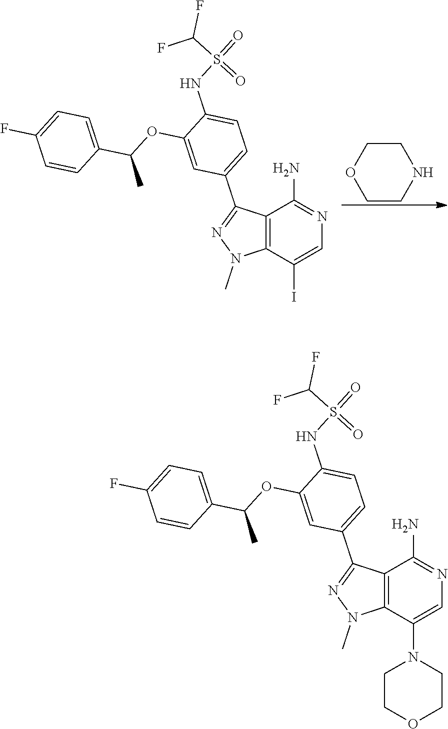 [SMILES: C[C@H](Oc1cc(-c2nn(C)c3c(I)cnc(N)c23)ccc1NS(=O)(=O)C(F)F)c1ccc(F)cc1 ; C1COCCN1 ; C[C@H](Oc1cc(-c2nn(C)c3c(N4CCOCC4)cnc(N)c23)ccc1NS(=O)(=O)C(F)F)c1ccc(F)cc1]

### Step 1: N-{4-[4-amino-1-methyl-7-(morpholin-4-yl)-1H-pyrazolo[4,3-c]pyridin-3-yl]-2-[(1S)-1-(4-fluorophenyl)ethoxy]phenyl}-1,1-difluoromethanesulfonamide

A mixture of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (300 mg, 485 μmol), morpholine (422 mg, 4.85 mmol) and K₂CO₃ (200 mg, 1.45 mmol) in ethylene glycol (3 mL) was stirred at 130° C. overnight. The reaction mixture was concentrated under reduced pressure and the crude mixture was purified by Prep-TLC (DCM/MeOH: 99/1) to give the title compound (8 mg, 3%) as a yellow solid. LCMS (Method A): 3.04 min, m/z: 577.1 [M+H]⁺. ¹H NMR (400 MHz, CDCl₃): 7.67 (d, J=8.2 Hz, 1H), 7.50 (s, 1H), 7.35-7.29 (m, 2H), 7.16 (dd, J=8.2, 1.8 Hz, 1H), 7.07-6.99 (m, 3H), 6.35 (t, J=53.6 Hz, 1H), 5.83 (s, 2H), 5.46 (q, J=6.3 Hz, 1H), 4.35 (s, 3H), 3.97 (d, J=9.4 Hz, 2H), 3.82-3.67 (m, 2H), 3.00 (d, J=16.7 Hz, 4H), 1.68 (d, J=6.4 Hz, 3H).

### Compound 133: N-(4-{4-amino-7-cyano-1-methyl-1H-pyrazolo[4,3-c]pyridin-3-yl}-2-[(1S)-1-(4-fluorophenyl)ethoxy]phenyl)-1,1-difluoromethanesulfonamide

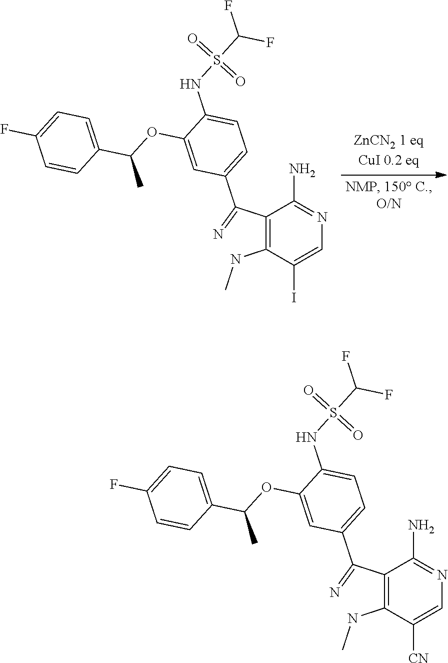 [SMILES: CNc1c(I)cnc(N)c1C(=N)c1ccc(NS(=O)(=O)C(F)F)c(O[C@@H](C)c2ccc(F)cc2)c1 ; CNc1c(C#N)cnc(N)c1C(=N)c1ccc(NS(=O)(=O)C(F)F)c(O[C@@H](C)c2ccc(F)cc2)c1]

### Step 1: N-(4-{4-amino-7-cyano-1-methyl-1H-pyrazolo[4,3-c]pyridin-3-yl}-2-[(1S)-1-(4-fluorophenyl)ethoxy]phenyl)-1,1-difluoromethanesulfonamide

A mixture of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (50 mg, 80.9 μmol), Cul (15.4 mg, 80.9 μmol) and Zn(CN)₂ (9.49 mg, 80.9 μmol) in NMP (4 mL) was stirred at 130° C. under N₂ overnight. The reaction was poured into EtOAc (20 mL), then the organics were washed with water (20 mL×3) and brine (10 mL), dried over Na₂SO₄ and concentrated under reduced pressure. The residue was purified by Prep-TLC (DCM/MeOH=15/1) to give the title product (7 mg, 17%) as a brown solid. LCMS (Method A): 3.69 min, m/z 517.0 [M+H]⁺. ¹H NMR (400 MHz, CDCl₃): 8.21 (s, 1H), 7.70 (d, J=8.5 Hz, 1H), 7.32 (t, J=6.9 Hz, 2H), 7.18 (d, J=8.5 Hz, 1H), 7.06 (t, J=8.4 Hz, 2H), 7.01 (s, 1H), 6.35 (t, J=52 Hz, 1H), 5.47 (q, J=6.6 Hz, 1H), 5.30 (s, 2H), 4.27 (s, 3H), 1.71 (d, J=6.4 Hz, 3H).

### Compound 44: (S)—N-(4-(4-amino-1-methyl-7-(1-(tetrahydro-2H-pyran-4-yl)-1H-pyrazol-4-yl)-1H-pyrazolo[4,3-c]pyridin-3-yl)-2-(1-(4-fluorophenyl)ethoxy)phenyl)-2,2,2-trifluoroethane-1-sulfonamide

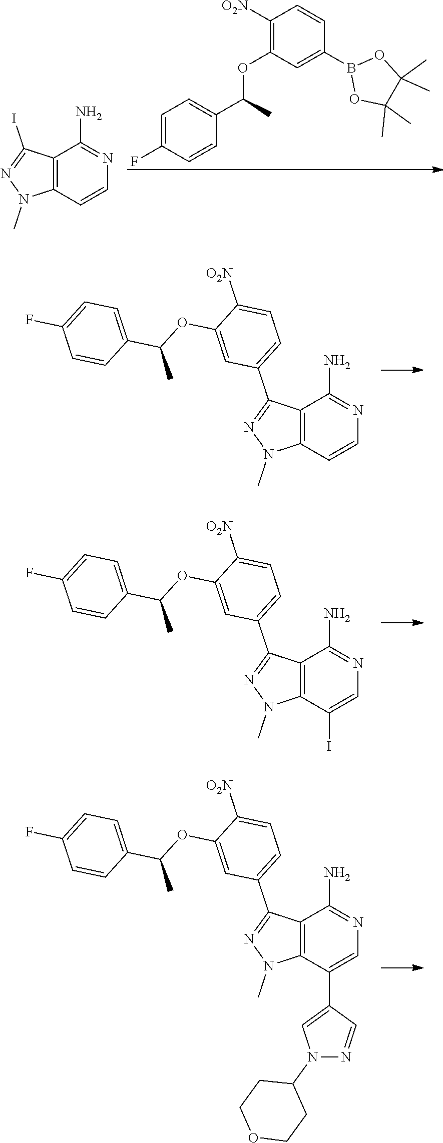

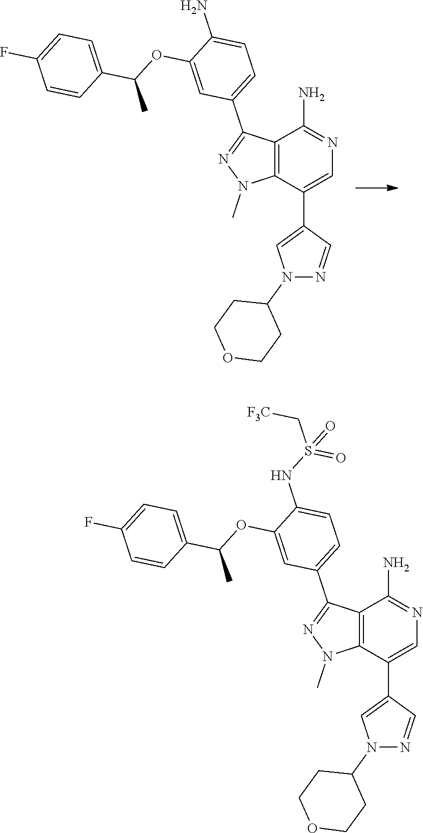 [SMILES: C[C@H](Oc1cc(-c2nn(C)c3c(-c4cnn(C5CCOCC5)c4)cnc(N)c23)ccc1N)c1ccc(F)cc1 ; C[C@H](Oc1cc(-c2nn(C)c3c(-c4cnn(C5CCOCC5)c4)cnc(N)c23)ccc1NS(=O)(=O)CC(F)(F)F)c1ccc(F)cc1]

### Step 1: (S)-3-(3-(1-(4-fluorophenyl)ethoxy)-4-nitrophenyl)-1-methyl-1H-pyrazolo[4,3-c]pyridin-4-amine

A mixture of 3-iodo-1-methyl-1H-pyrazolo[4,3-c]pyridin-4-amine (4 g, 14.5 mmol), (S)-2-(3-(1-(4-fluorophenyl)ethoxy)-4-nitrophenyl)-4,4,5,5-tetramethyl-1,3,2-dioxaborolane (5.61 g, 14.5 mmol), K₂CO₃ (6.01 g, 43.5 mmol) and Pd(dppf)Cl₂ (1.18 g, 1.45 mmol) in degassed 1,4-dioxane/H₂O (4/1, 125 mL) was stirred at 100° C. for 10 h under N₂. The mixture was concentrated under reduced pressure and the residue was purified by column chromatography (PE/EtOAc=4:1 to 1:4) to give the title product (5.2 g, 88%) as a yellow solid. LCMS (Method A): 3.31 min, m/z: 408.4 [M+H]⁺.

### Step 2: (S)-3-(3-(1-(4-fluorophenyl)ethoxy)-4-nitrophenyl)-7-iodo-1-methyl-1H-pyrazolo[4,3-c]pyridin-4-amine

A mixture of (S)-3-(3-(1-(4-fluorophenyl)ethoxy)-4-nitrophenyl)-1-methyl-1H-pyrazolo[4,3-c]pyridin-4-amine (2.5 g, 6.13 mmol) and NIS (2.74 g, 12.2 mmol) in DMF (50 mL) was stirred at 85° C. for 6 h under N₂. The mixture was concentrated under reduced pressure and the residue was purified by column chromatography (PE/EtOAc=4:1 to 1:4) to give the title product (1.1 g, 33%) as a yellow solid. LCMS (Method A): 3.51 min, m/z: 534.3 [M+H]⁺.

### Step 3: (S)-3-(3-(1-(4-fluorophenyl)ethoxy)-4-nitrophenyl)-1-methyl-7-(1-(tetrahydro-2H-pyran-4-yl)-1H-pyrazol-4-yl)-1H-pyrazolo[4,3-c]pyridin-4-amine

A mixture of (S)-3-(3-(1-(4-fluorophenyl)ethoxy)-4-nitrophenyl)-7-iodo-1-methyl-1H-pyrazolo[4,3-c]pyridin-4-amine (1.1 g, 2.06 mmol), 1-(tetrahydro-2H-pyran-4-yl)-4-(4,4,5,5-tetramethyl-1,3,2-dioxaborolan-2-yl)-1H-pyrazole (573 mg, 2.06 mmol), K₂CO₃ (854 mg, 6.18 mmol) and Pd(dppf)Cl₂ (168 mg, 206 μmol) in 1,4-dioxane/H₂O (4/1, 25 mL) was stirred at 100° C. for 10 h under N₂. The mixture was concentrated under reduced pressure and the residue was purified was purified by column chromatography (PE/EtOAc=4:1 to 1:4) to give the title product (0.9 g, 76%) as a yellow solid. LCMS (Method A): 3.10 min, m/z: 558.6 [M+H]⁺.

### Step 4: (S)-3-(4-amino-3-(1-(4-fluorophenyl)ethoxy)phenyl)-1-methyl-7-(1-(tetrahydro-2H-pyran-4-yl)-1H-pyrazol-4-yl)-1H-pyrazolo[4,3-c]pyridin-4-amine

A mixture of (S)-3-(3-(1-(4-fluorophenyl)ethoxy)-4-nitrophenyl)-1-methyl-7-(1-(tetrahydro-2H-pyran-4-yl)-1H-pyrazol-4-yl)-1H-pyrazolo[4,3-c]pyridin-4-amine (0.9 g, 1.61 mmol) and Zn dust (525 mg, 8.04 mmol) in MeOH (18 mL) and sat. aq. NH₄Cl (6 mL) was stirred at 60° C. for 8 h. The mixture was poured into water (20 mL) and the organics were extracted with DCM (2×10 mL). The combined organic phases were dried over Na₂SO₄ and concentrated under reduced pressure. The residue was purified by column chromatography (PE/EtOAc=1:4 to 1:10) to give the title product (330 mg 39%) as a white solid. LCMS (Method A): 2.85 min, m/z: 528.6 [M+H]⁺.

### Step 5: (S)—N-(4-(4-amino-1-methyl-7-(1-(tetrahydro-2H-pyran-4-yl)-1H-pyrazol-4-yl)-1H-pyrazolo[4,3-c]pyridin-3-yl)-2-(1-(4-fluorophenyl)ethoxy)phenyl)-2,2,2-trifluoroethane-1-sulfonamide

A solution of (S)-3-(4-amino-3-(1-(4-fluorophenyl)ethoxy)phenyl)-1-methyl-7-(1-(tetrahydro-2H-pyran-4-yl)-1H-pyrazol-4-yl)-1H-pyrazolo[4,3-c]pyridin-4-amine (100 mg, 189 μmol) and 2,2,2-trifluoroethanesulfonyl chloride (51.6 mg, 283 μmol) in DCM (2.5 mL) and pyridine (0.5 mL) was stirred at 35° C. for 10 h. The mixture was concentrated under reduced pressure and the residue was purified by column chromatography (DCM/MeOH=20/1) to give the title product (7 mg, 6%) as a white solid. LCMS (Method A): 3.17 min, m/z: 674.7 [M+H]⁺. ¹H NMR (400 MHz, CDCl₃): 7.71 (d, J=8.2 Hz, 1H), 7.62 (s, 1H), 7.58 (s, 1H), 7.54 (s, 1H), 7.36-7.28 (m, 2H), 7.25-7.20 (m, 1H), 7.11-7.00 (m, 3H), 5.47 (q, J=6.4 Hz, 1H), 4.43 (tt, J=10.4, 4.8 Hz, 1H), 4.15 (dt, J=11.8, 3.1 Hz, 2H), 3.92 (q, J=8.7 Hz, 2H), 3.77 (s, 3H), 3.59 (td, J=11.6, 2.8 Hz, 2H), 2.26-2.09 (m, 4H), 1.69 (d, J=6.4 Hz, 3H).

The following compounds were prepared similarly to the synthesis of (S)—N-(4-(4-amino-1-methyl-7-(1-(tetrahydro-2H-pyran-4-yl)-1H-pyrazol-4-yl)-1H-pyrazolo[4,3-c]pyridin-3-yl)-2-(1-(4-fluorophenyl)ethoxy)phenyl)-2,2,2-trifluoroethane-1-sulfonamide (compound 44) with the indicated sulfonyl chloride in step 5.

| Com- | | | | | | |
|---|---|---|---|---|---|---|
| pound | | | | LCMS | | Sulfonyl |
| No | Name | Structure | Yield | data | ¹H NMR data | chloride |
| 45 | (S)-N-(4-(4-amino-1- methyl-7-(1-(tetrahydro-2H- pyran-4-yl)-1H-pyrazol- 4-yl)-1H-pyrazolo[4,3- c]pyridin-3-yl)-2-(1-(4- fluorophenyl)ethoxy)phe- nyl)ethanesulfonamide | 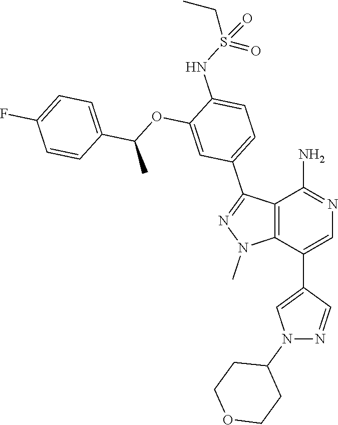 [SMILES: CCS(=O)(=O)Nc1ccc(-c2nn(C)c3c(-c4cnn(C5CCOCC5)c4)cnc(N)c23)cc1O[C@@H](C)c1ccc(F)cc1] | 26% | LCMS (Method A): 2.97 min, m/z: 620.7 [M + H]⁺. | (400 MHz, DMSO-d₆): 9.24 (s, 1H), 8.04 (s, 1H), 7.64-7.55 (m, 4H), 7.45 (d, J = 7.9 Hz, 1H), 7.17 (t, J = 8.9 Hz, 2H), 7.12 (s, 2H), 5.89 (s, 2H), 5.66 (d, J = 6.3 Hz, 1H), 4.47 (tt, J = 10.4, 5.3 Hz, 1H), 4.03-3.94 (m, 2H), 3.70 (s, 3H), 3.51 (dd, J = 11.2, 3.2 Hz, 2H), 3.16-3.10 (m, 2H), 2.02 (q, J = 4.3 Hz, 4H), 1.60 (d, J = 6.2 Hz, 3H), 1.34-1.28 (m, 3H). | ethane- sulfonyl chloride |
| 119 | (S)-N-(4-(4-amino-1- methyl-7-(1-(tetrahydro-2H- pyran-4-yl)-1H-pyrazol- 4-yl)-1H-pyrazolo[4,3- c]pyridin-3-yl)-2-(1-(4- fluorophenyl)ethoxy)phe- nyl)methanesulfonamide | 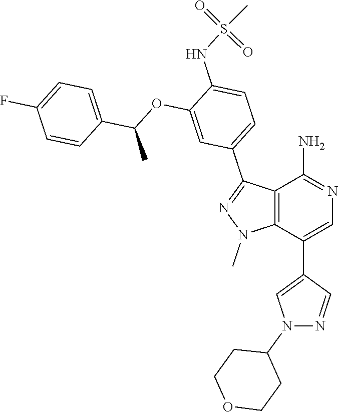 [SMILES: C[C@H](Oc1cc(-c2nn(C)c3c(-c4cnn(C5CCOCC5)c4)cnc(N)c23)ccc1NS(C)(=O)=O)c1ccc(F)cc1] | 43% | LCMS (Method A): 3.04 min, m/z: 606.1 [M + H]⁺. | (400 MHz, DMSO-d₆): 9.22 (s, 1H), 8.05 (s, 1H), 7.65-7.56 (m, 4H), 7.43 (d, J = 8.3 Hz, 1H), 7.21-7.10 (m, 4H), 6.34-6.05 (m, 2H), 5.65 (q, J = 6.3 Hz, 1H), 4.47 (tt, J = 10.4, 5.3 Hz, 1H), 3.98 (d, J = 10.5 Hz, 2H), 3.70 (s, 3H), 3.49 (td, J = 11.4, 3.4 Hz, 3H), 3.05 (s, 3H), 2.01 (tt, J = 11.6, 5.6 Hz, 4H), 1.60 (d, J = 6.2 Hz, 3H). | methane- sulfonyl chloride |
| 124 | 3-{4-[(dimethyl- sulfamoyl)amino]-3- [(1S)-1-(4-fluoro- phenyl)ethoxy]phenyl}- 1-methyl-7-[1-9oxan- 4-yl)-1H-pyrazol-4-yl]- 1H-pyrazolo[4,3- c]pyridin-4-amine | 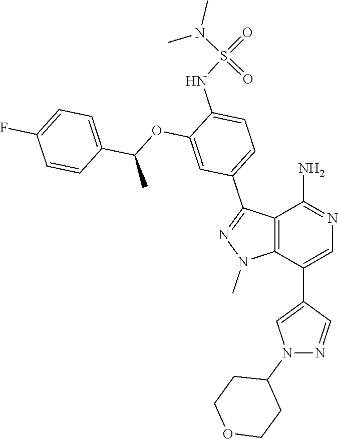 [SMILES: C[C@H](Oc1cc(-c2nn(C)c3c(-c4cnn(C5CCOCC5)c4)cnc(N)c23)ccc1NS(=O)(=O)N(C)C)c1ccc(F)cc1] | 78% | LCMS (Method A): 3.03 min, m/z: 635.1 [M + H]⁺. | (400 MHz, DMSO-d₆): 9.16 (s, 1H), 8.00 (s, 1H), 7.61 (d, J = 6.3 Hz, 3H), 7.54 (s, 1H), 7.49 (d, J = 8.0 Hz, 1H), 7.16 (t, J = 8.9 Hz, 2H), 7.09 (d, J = 10.3 Hz, 2H), 5.67 (q, J = 6.2 Hz, 1H), 5.60 (s, 2H), 4.46 (tt, J = 9.8, 4.7 Hz, 1H), 4.01-3.92 (m, 2H), 3.69 (s, 3H), 3.49 (td, J = 11.0, 3.3 Hz, 2H), 2.73 (s, 6H), 2.09-1.98 (m, 4H), 1.62 (d, J = 6.2 Hz, 3H). | N,N- dimethyl- sulfamoyl chloride |
| 125 | (S)-N-(4-(4-amino- 1-methyl-7-(1-(tetra- hydro-2H-pyran-4-yl)- 1H-pyrazol-4-yl)- 1H-pyrazolo[4,3- c]pyridin-3-yl)- 2-(1-(4-fluoro- phenyl)eth- oxy)phenyl)-1- phenylmethane- sulfonamide | 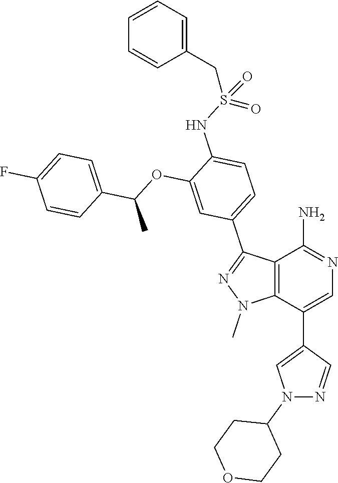 [SMILES: C[C@H](Oc1cc(-c2nn(C)c3c(-c4cnn(C5CCOCC5)c4)cnc(N)c23)ccc1NS(=O)(=O)Cc1ccccc1)c1ccc(F)cc1] | | LCMS (Method A): 3.32 min; m/z: 682.0 [M + H]+. | (400 MHz, DMSO-d₆): 9.18 (s, 1H), 8.02 (s, 1H), 7.61-7.58 (m, 2H), 7.55 (s, 1H), 7.37-7.34 (m, 6H), 7.17 (t, J = 8.8 Hz, 2H), 7.11 (s, 1H), 7.04 (d, J = 8 Hz, 1H), 5.67 (q, J = 6.4 Hz, 3H), 4.54 (s, 2H), 4.50-4.43 (m, 1H), 3.99-3.93 (m, 2H), 3.69 (s, 3H), 3.52-3.46 (m, 2H), 2.04-1.98 (m, 4H), 1.62 (d, J = 6 Hz, 3H). | phenyl- methane- sulfonyl chloride |

### General Procedure C₁ for the Cross Coupling Reaction Between Intermediate A1B1 and Halogenopyrazoles D

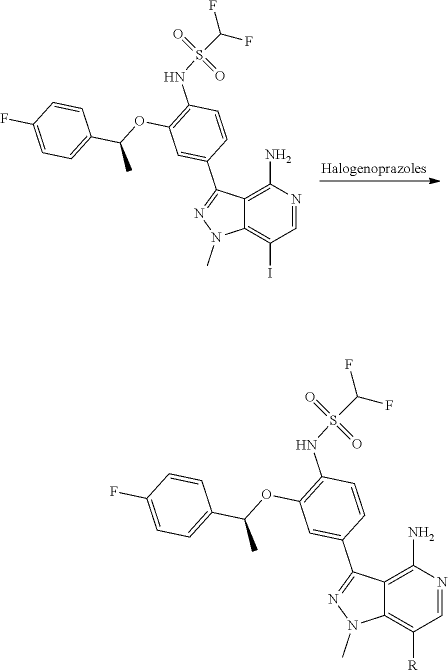

A mixture of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (1 eq), halogenopyrazole (1.0 eq), B₂(pin)₂ (2 eq), Pd(OAc)₂ (0.2 eq) bis(adamantan-1-yl)(butyl)phosphane (0.4 eq) and K₂CO₃ (2 eq) were dissolved in degassed 1,4-dioxane/H₂O (4/1, 0.2 M) and the solution was stirred at 100° C. under N₂ overnight (or at 80° C. for 2 h under μW irradiation). The mixture was diluted with water and extracted with EtOAc (3×). The combined organics were dried over Na₂SO₄ and concentrated under reduced pressure. The residue was purified by column chromatography (DCM/MeOH or EtOAc/PE) or prep TLC (DCM/MeOH)

The following compounds were prepared following the General procedure C1

| Compound | Name | Structure | LCMS data | ¹H NMR data | Intermediate D |
|---|---|---|---|---|---|
| 145 | N-(4-(4-amino-1-methyl-7-(1-(4- methyl-4-azaspiro[2.5]octan-7-yl)-1H- pyrazol-4-yl)-1H-pyrazolo[4,3- c]pyridin-3-yl)-2-((S)-1-(4- fluorophenyl)ethoxy)phenyl)-1,1- difluoromethanesulfonamide | 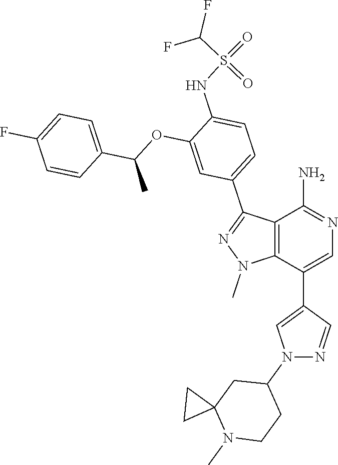 [SMILES: C[C@H](Oc1cc(-c2nn(C)c3c(-c4cnn(C5CCN(C)C6(CC6)C5)c4)cnc(N)c23)ccc1NS(=O)(=O)C(F)F)c1ccc(F)cc1] | (Method A): 2.31 min, m/z 681.1 [M + H]⁺ | (400 MHz, CDCl₃): 8.09 (s, 1H), 7.59-7.53 (m, 3H), 7.39 (d, J = 8 Hz, 1H), 7.18-7.02 (m, 4H), 6.89 (t, J = 52 Hz, 1H), 5.67-5.63 (m, 3H), 4.45-4.37 (m, 1H), 3.69 (s, 3H), 2.97-2.89 (m, 2H), 2.39 (s, 3H), 2.23-2.15 (m, 1H), 2.02 (s, 1H), 1.82-1.74 (m, 2H), 1.57 (d, J = 8 Hz, 3H), 1.25-1.23 (m, 1H), 0.66-0.40 (m, 4H). | D2 |
| 146 | (S)-N-(4-(4-amino-7-(1-(4,4- difluorocyclohexyl)-1H-pyrazol-4-yl)-1- methyl-1H-pyrazolo[4,3-c]pyridin-3-yl)- 2-(1-(4-fluorophenyl)ethoxy)phenyl)- 1,1-difluoromethanesulfonamide | 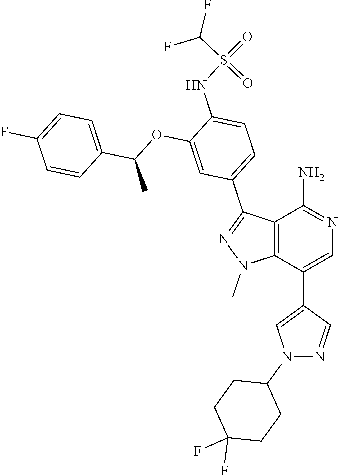 [SMILES: C[C@H](Oc1cc(-c2nn(C)c3c(-c4cnn(C5CCC(F)(F)CC5)c4)cnc(N)c23)ccc1NS(=O)(=O)C(F)F)c1ccc(F)cc1] | (Method A): 3.00 min, m/z: 676.2 [M + H]⁺ | (400 MHz, DMSO-d₆): 8.02 (s, 1H), 7.62-7.55 (m, 4H), 7.40 (d, J = 8 Hz, 1H), 7.19-7.11 (m, 4H), 7.00 (d, J = 52 Hz, 1H), 5.76 (s, 2H), 5.65 (q, J = 8 Hz, 1H), 4.46 (s, 1H), 3.69 (s, 3H), 2.17-1.99 (m, 8H), 1.58 (d, J = 8 Hz, 3H). | D3 |
| 152 | N-(4-(4-amino-7-(1-(2,2-dimethyl-1- (methyl-d3)piperidin-4-yl)-1H-pyrazol- 4-yl)-1-methyl-1H-pyrazolo[4,3- c]pyridin-3-yl)-2-((S)-1-(4- fluorophenyl)ethoxy)phenyl)-1,1- difluoromethanesulfonamide | 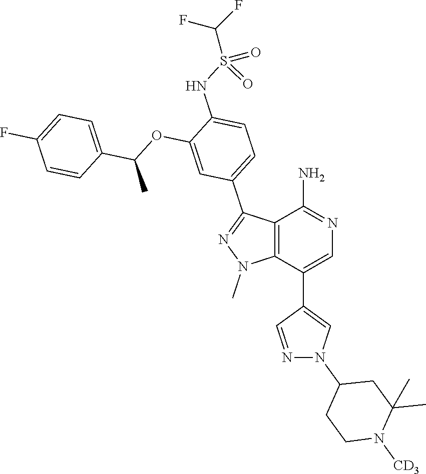 [SMILES: [2H]C([2H])([2H])N1CCC(n2cc(-c3cnc(N)c4c(-c5ccc(NS(=O)(=O)C(F)F)c(O[C@@H](C)c6ccc(F)cc6)c5)nn(C)c34)cn2)CC1(C)C] | (Method A): 2.29 min, m/z: 686.2 [M + H]⁺ | (400 MHz, DMSO-d₆): 7.95 (s, 1H), 7.62 (s, 1H), 7.55-7.51 (m, 3H), 7.37 (d, J = 8 Hz, 1H), 7.15 (t, J = 8 Hz, 1H), 7.04-6.96 (m, 2H), 6.69 (t, J = 52 Hz, 1H), 5.67-5.64 (m, 3H), 4.59 (s, 1H), 3.67 (s, 3H), 3.09-3.01 (m, 2H), 2.17-2.06 (m, 4H), 1.55 (d, J = 4 Hz, 3H), 1.27 (d, J = 20 Hz, 6H) | D4 |
| 153 | N-(4-(4-amino-1-methyl-7-(1-(8-(2,2,2- trifluoroethyl)-8- azabicyclo[3.2.1]octan-3-yl)-1H- pyrazol-4-yl)-1H-pyrazolo[4,3- c]pyridin-3-yl)-2-((S)-1-(4- fluorophenyl)ethoxy)phenyl)-1,1- difluoromethanesulfonamide | 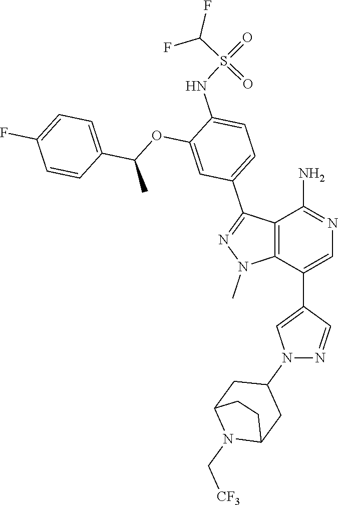 [SMILES: C[C@H](Oc1cc(-c2nn(C)c3c(-c4cnn(C5CC6CCC(C5)N6CC(F)(F)F)c4)cnc(N)c23)ccc1NS(=O)(=O)C(F)F)c1ccc(F)cc1] | (Method A): 2.99 min, m/z: 749.2 [M + H]⁺ | (400 MHz, DMSO-d₆): 8.17 (s, 1H), 7.64-7.51 (m, 4H), 7.41 (d, J = 8.0 Hz, 1H), 7.22-7.07 (m, 5H), 6.93 (d, J = 52.6 Hz, 1H), 5.65 (d, J = 6.3 Hz, 1H), 4.39 (t, J = 3.2 Hz, 1H), 3.70 (d, J = 11.1 Hz, 3H), 3.08 (d, J = 10.1 Hz, 2H), 2.61- 2.52 (m, 2H), 2.32 (dt, J = 14.2, 4.1 Hz, 2H), 2.19-2.07 (m, 1H), 2.04-1.86 (m, 2H), 1.81- 1.69 (m, 2H), 1.58 (d, J = 6.2 Hz, 3H), 1.47 (d, J = 7.7 Hz, 2H), 0.91-0.75 (m, 1H) | D5 |
| 156 | N-(4-(4-amino-1-methyl-7-(3-methyl-1- (1,2,2-trimethylpiperidin-4-yl)-1H- pyrazol-4-yl)-1H-pyrazolo[4,3- c]pyridin-3-yl)-2-((S)-1-(4- fluorophenyl)ethoxy)phenyl)-1,1- difluoromethanesulfonamide | 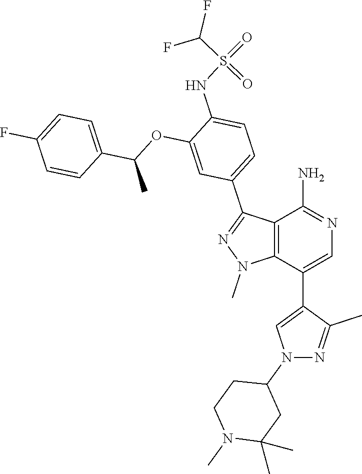 [SMILES: Cc1nn(C2CCN(C)C(C)(C)C2)cc1-c1cnc(N)c2c(-c3ccc(NS(=O)(=O)C(F)F)c(O[C@@H](C)c4ccc(F)cc4)c3)nn(C)c12] | (Method A): 2.35 min, m/z: 697.3 [M + H]⁺ | (400 MHz, DMSO-d₆): 10.81 (s, 1H), 7.60-7.56 (m, 3H), 7.50 (s, 1H), 7.41 (d, J = 4 Hz, 1H), 7.20-7.14 (m, 4H), 7.02 (t, J = 52 Hz, 1H), 6.34 (s, 2H), 5.66 (q, J = 5.2 Hz, 1H), 4.70-4.63 (m, 1H), 3.58 (s, 3H), 3.38-3.35 (m, 2H), 2.67 (s, 3H), 2.21 (s, 3H), 2.08-2.05 (m, 2H), 1.58 (d, J = 4 Hz, 3H), 1.44 (d, J = 16 Hz, 6H), 1.30-1.21 (m, 2H) | D6 |
| 157 | N-(4-(4-amino-1-methyl-7-(5-methyl-1- (1,2,2-trimethylpiperidin-4-yl)-1H- pyrazol-4-yl)-1H-pyrazolo[4,3- c]pyridin-3-yl)-2-((S)-1-(4- fluorophenyl)ethoxy)phenyl)-1,1- difluoromethanesulfonamide | 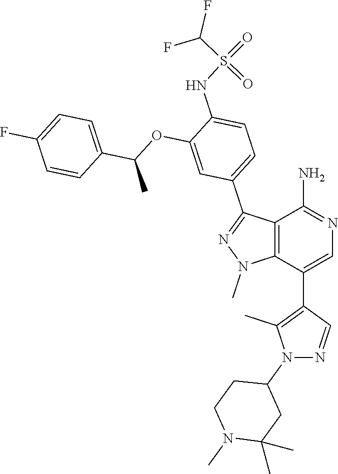 [SMILES: Cc1c(-c2cnc(N)c3c(-c4ccc(NS(=O)(=O)C(F)F)c(O[C@@H](C)c5ccc(F)cc5)c4)nn(C)c23)cnn1C1CCN(C)C(C)(C)C1] | (Method A): 2.29 min, m/z: 697.3 [M + H]⁺ | (400 MHz, DMSO-d₆): 7.81 (s, 1H), 7.59-7.55 (m, 2H), 7.49 (s, 1H), 7.40 (d, J = 8 Hz, 1H), 7.19-7.11 (m, 4H), 6.95 (t, J = 52 Hz, 1H), 5.77 (s, 2H), 5.66 (q, J = 6.8 Hz, 1H), 4.64-4.57 (m, 1H), 3.58 (s, 3H), 3.38-3.35 (m, 2H), 2.65 (s, 3H), 2.31-2.16 (m, 4H), 2.05 (s, 3H), 1.58 (d, J = 4 Hz, 3H), 1.39 (d, J = 16 Hz, 6H) | D7 |
| 158 | N-(4-(4-amino-7-(1-(2-(difluoromethyl)- 1-methylpiperidin-4-yl)-1H-pyrazol-4- yl)-1-methyl-1H-pyrazolo[4,3-c]pyridin- 3-yl)-2-((S)-1-(4- fluorophenyl)ethoxy)phenyl)-1,1- difluoromethanesulfonamide | 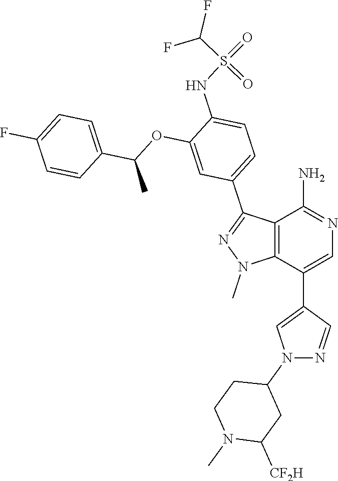 [SMILES: C[C@H](Oc1cc(-c2nn(C)c3c(-c4cnn(C5CCN(C)C(C(F)F)C5)c4)cnc(N)c23)ccc1NS(=O)(=O)C(F)F)c1ccc(F)cc1] | (Method A): 2.36 min, m/z: 706.2 [M + H]⁺ | (400 MHz, DMSO-d₆): 8.01 (s, 1H), 7.62 (s, 1H), 7.58-7.55 (m, 3H), 7.40 (d, J = 8 Hz, 1H), 7.19-7.10 (m, 4H), 6.98 (t, J = 52 Hz, 1H), 6.25 (m, 1H), 5.73 (s, 2H), 5.65 (q, J = 6.8 Hz, 1H), 4.37-4.28 (m, 1H), 3.69 (s, 3H), 2.95 (d, J = 12 Hz, 1H), 2.58-2.53 (m, 1H), 2.37-2.33 (m, 1H), 2.32 (s, 3H), 2.22-1.82 (m, 5H), 1.58 (d, J = 4 Hz, 3H) | D8 |
| 162 | (S)-N-(4-(4-amino-1-methyl-7-(1- (1,2,2,6,6-pentamethylpiperidin-4-yl)- 1H-pyrazol-4-yl)-1H-pyrazolo[4,3- c]pyridin-3-yl)-2-(1-(4- fluorophenyl)ethoxy)phenyl)-1,1- difluoromethanesulfonamide | 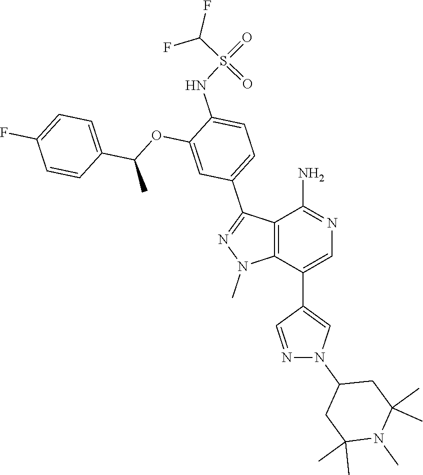 [SMILES: C[C@H](Oc1cc(-c2nn(C)c3c(-c4cnn(C5CC(C)(C)N(C)C(C)(C)C5)c4)cnc(N)c23)ccc1NS(=O)(=O)C(F)F)c1ccc(F)cc1] | (Method A): 2.30 min, m/z: 711.3 [M + H]⁺ | (400 MHz, DMSO-d₆): 7.98 (s, 1H), 7.62 (s, 1H), 7.55-7.51 (m, 3H), 7.37 (d, J = 8 Hz, 1H), 7.17-7.12 (m, 2H), 7.04-6.98 (m, 2H), 6.68 (t, J = 52 Hz, 1H), 5.67-5.63 (m, 3H), 4.74 (s, 1H), 3.68 (s, 3H), 2.45 (s, 3H), 2.09-2.02 (m, 4H), 1.54 (d, J = 4 Hz, 3H), 1.26 (d, J = 12 Hz, 12H) | D9 |
| 167 | N-(4-(4-amino-7-(1-(2,2-dimethyl-1- (2,2,2-trifluoroethyl)piperidin-4-yl)-1H- pyrazol-4-yl)-1-methyl-1H- pyrazolo[4,3-c]pyridin-3-yl)-2-((S)-1-(4- fluorophenyl)ethoxy)phenyl)-1,1- difluoromethanesulfonamide | 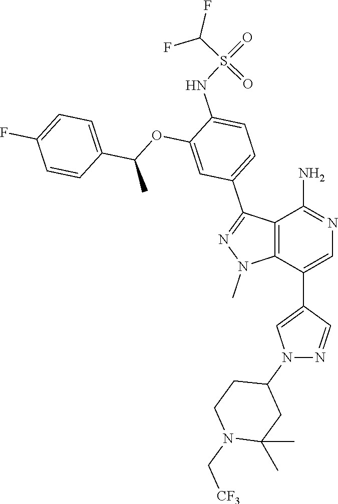 [SMILES: C[C@H](Oc1cc(-c2nn(C)c3c(-c4cnn(C5CCN(CC(F)(F)F)C(C)(C)C5)c4)cnc(N)c23)ccc1NS(=O)(=O)C(F)F)c1ccc(F)cc1] | (Method A): 3.12 min, m/z: 751.3 [M + H]⁺ | (400 MHz, DMSO-d₆): 10.68 (s, 1H), 8.01 (s, 1H), 7.59-7.54 (m, 4H), 7.40 (d, J = 8 Hz, 1H), 7.18-7.09 (m, 4H), 6.96 (t, J = 52 Hz, 1H), 5.87 (s, 2H), 5.65 (q, J = 6.4 Hz, 1H), 4.49-4.43 (m, 1H), 3.68 (s, 3H), 3.56-3.52 (m, 1H), 2.88- 2.73 (m, 3H), 2.02-1.86 (m, 4 H), 1.57 (d, J = 6 Hz, 3H), 1.15 (s, 3H), 1.06 (s, 3H) | D10 |
| 154 | N-(4-(4-amino-1-methyl-7-(1-((R*)- 1,2,2-trimethylpiperidin-4-yl)-1H- pyrazol-4-yl)-1H-pyrazolo[4,3- c]pyridin-3-yl)-2-((S)-1-(4- fluorophenyl)ethoxy)phenyl)-1,1- difluoromethanesulfonamide | 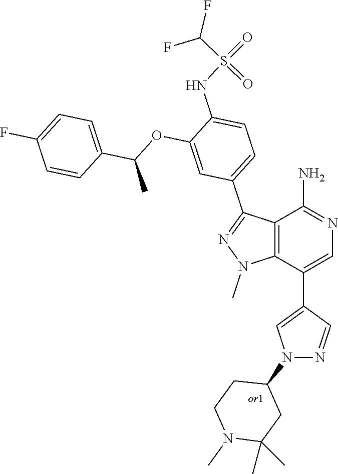 [SMILES: C[C@H](Oc1cc(-c2nn(C)c3c(-c4cnn([C@@H]5CCN(C)C(C)(C)C5)c4)cnc(N)c23)ccc1NS(=O)(=O)C(F)F)c1ccc(F)cc1] | (Method A): 2.36 min, m/z: 683.3 [M + H]⁺ | (400 MHz, DMSO-d₆): 7.98 (s, 1H), 7.66 (s, 1H), 7.58-7.54 (m, 3H), 7.40 (d, J = 8 Hz, 1H), 7.19-7.08 (m, 4H), 6.97 (t, J = 52 Hz, 1H), 5.77 (s, 2H), 5.65 (q, J = 5.2 Hz, 1H), 4.73-4.67 (m, 1H), 3.69 (s, 3H), 3.39-3.35 (m, 2H), 2.66 (s, 3H), 2.35-2.20 (m, 4H), 1.58 (d, J = 8 Hz, 3H), 1.40 (d, J = 16 Hz, 6H) | D11 |
| 155 | N-(4-(4-amino-1-methyl-7-(1-((S*)- 1,2,2-trimethylpiperidin-4-yl)-1H- pyrazol-4-yl)-1H-pyrazolo[4,3- c]pyridin-3-yl)-2-((S)-1-(4- fluorophenyl)ethoxy)phenyl)-1,1- difluoromethanesulfonamide | 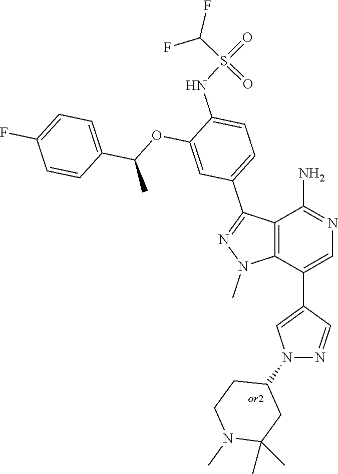 | (Method A): 2.34 min, m/z: 683.1 [M + H]⁺ | (400 MHz, DMSO-d₆): 7.99 (s, 1H), 7.66 (s, 1H), 7.58-7.55 (m, 3H), 7.41 (d, J = 8 Hz, 1H), 7.19-7.09 (m, 4H), 6.99 (t, J = 52 Hz, 1H), 5.83 (s, 2H), 5.65 (q, J = 5.2 Hz, 1H), 4.75-4.67 (m, 1H), 3.69 (s, 3H), 3.38-3.35 (m, 2H), 2.67 (s, 3H), 2.35-2.21 (m, 4H), 1.58 (d, J = 8 Hz, 3H), 1.42 (d, J = 16 Hz, 6H) | D12 |
| 170 | N-(4-(4-amino-1-methyl-7-(1-((S*)-4- methyl-4-azaspiro[2.5]octan-7-yl)-1H- pyrazol-4-yl)-1H-pyrazolo[4,3- c]pyridin-3-yl)-2-((S)-1-(4- fluorophenyl)ethoxy)phenyl)-1,1- difluoromethanesulfonamide | 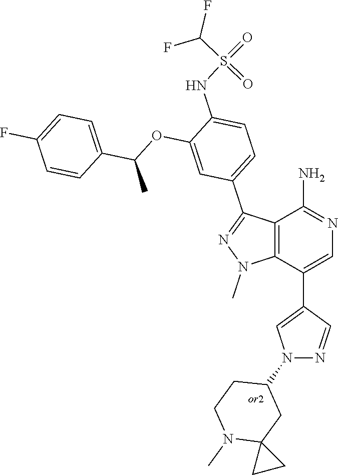 | (Method B): 1.45 min, m/z: 341.2 [M + 2H]²⁺ | (400 MHz, DMSO-d₆): 8.03 (s, 1H), 7.59 (s, 1H), 7.60-7.55 (m, 3H), 7.39 (d, J = 8 Hz, 1H), 7.16 (m, 2H), 7.09 (m, 2H), 6.93 (t, J = 52 Hz, 1H), 5.70-5.63 (m, 3H), 4.47-4.41 (m, 1H), 3.69 (s, 3H), 3.06-2.98 (m, 2H), 2.56 (m, 1H), 2.45 (s, 3H), 2.24 (m, 1H), 1.88-1.85 (m, 2H), 1.57 (d, J = 8 Hz, 3H), 1.30 (m, 1H), 0.70 (m, 2H) 0.54-0.45 (m, 2H) | D13 |
| 171 | N-(4-(4-amino-1-methyl-7-(1-((R*)-4- methyl-4-azaspiro[2.5]octan-7-yl)-1H- pyrazol-4-yl)-1H-pyrazolo[4,3- c]pyridin-3-yl)-2-((S)-1-(4- fluorophenyl)ethoxy)phenyl)-1,1- difluoromethanesulfonamide | 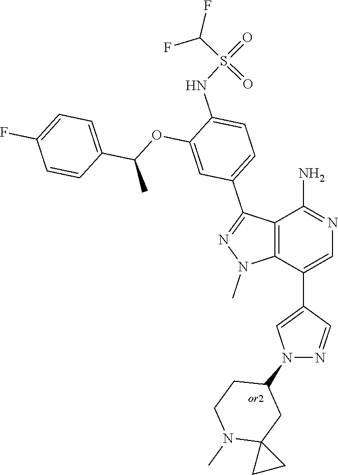 | (Method B): 1.42 min, m/z: 341.2 [M + 2H]²⁺ | (400 MHz, DMSO-d₆): 8.03 (s, 1H), 7.59 (s, 1H), 7.57-7.53 (m, 3H), 7.39 (d, J = 8 Hz, 1H), 7.15 (m, 2H), 7.07 (m, 2H), 6.87 (t, J = 52 Hz, 1H), 5.67-5.63 (m, 3H), 4.45-4.37 (m, 1H), 3.69 (s, 3H), 3.02-2.89 (m, 2H), 2.60 (m, 1H), 2.45 (s, 3H), 2.22 (m, 1H), 1.85-1.82 (m, 2H), 1.56 (d, J = 8 Hz, 3H), 1.31 (m, 1H), 0.70 (m, 2H), 0.55-0.46 (m, 2H) | D14 |
| 172 | N-(4-(4-amino-1-methyl-7-[1-(1,3,5- trimethylpiperidin-4-yl)-1H-pyrazol-4- yl]-1H-pyrazolo[4,3-c]pyridin-3-yl}-2- [(1S)-1-(4- fluorophenyl)ethoxy]phenyl)-1,1- difluoromethanesulfonamide | 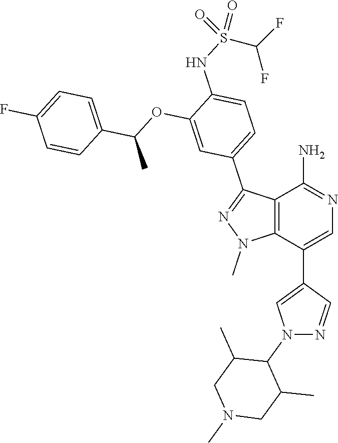 [SMILES: CC1CN(C)CC(C)C1n1cc(-c2cnc(N)c3c(-c4ccc(NS(=O)(=O)C(F)F)c(O[C@@H](C)c5ccc(F)cc5)c4)nn(C)c23)cn1] | (Method B): 1.76 min, m/z: 683.1 [M + H]⁺ | (400 MHz, DMSO) δ 10.94 (s, 1H), 8.02 (s, 1H), 7.75 (s, 1 H), 7.58-7.54 (m, 3H), 7.40 (d, J = 8 Hz, 1H), 7.18-7.09 (m, 5H), 6.99 (t, J = 52.8 Hz, 1H), 5.88 (s, 2H), 5.65 (q, J = 6.4 Hz, 1H), 4.48 (t, J = 4 Hz, 1H), 3.69 (s, 3H), 2.82 (s, 3H), 2.64-2.60 (m, 2H), 2.48 (s, 1H), 2.15 (s, 2H), 1.58 (d, J = 6 Hz, 3H), 0.67 (d, J = 6.8 Hz, 6H). | D15 |

### General Procedure C2 for the Cross Coupling Reaction Followed by Acidic Deprotection Between Intermediate A1B1 and Boc Protected Halogenopyrazoles D

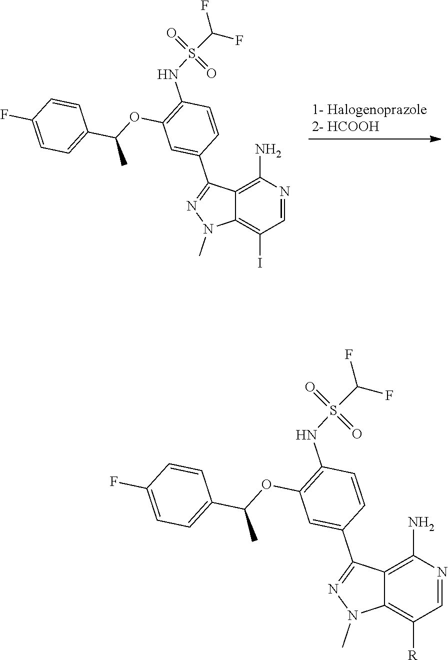

A mixture of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (1 eq), halogenopyrazole (1.0 eq), B₂(pin)₂ (2 eq), Pd(OAc)₂ (0.2 eq) bis(adamantan-1-yl)(butyl)phosphane (0.4 eq) and K₂CO₃ (2 eq) were dissolved in degassed 1,4-dioxane/H₂O (4/1, 0.2 M) and the solution was stirred at 100° C. under N₂ overnight (or at 80° C. for 2 h under μW irradiation). The mixture was diluted with water and extracted with EtOAc (3×). The combined organics were dried over Na₂SO₄ and concentrated under reduced pressure. The residue was purified by column chromatography (DCM/MeOH or EtOAc/PE) or prep-TLC (DCM/MeOH)

The solids were dissolved in HCOOH neat (2 M) and the reaction mixture was stirred at RT for 30 min or until completion. The reaction mixture was concentrated under reduced pressure and the residue was adjusted to pH=8 with sat. aq. Na₂CO₃. The residue was further diluted with water and the organics were extracted with EtOAc (3 times). The combined organics were washed with brine, dried over Na₂SO₄ and concentrated under reduced pressure. The residue was purified by prep-TLC (DCM/MeOH) followed by preparative HPLC (neutral condition method C) if needed to give the desired products

The following compounds were prepared following the General procedure C2

| | | | | | Inter - |
|---|---|---|---|---|---|
| Com- | | | LCMS | | mediate |
| pound | Name | Structure | data | ¹H NMR data | D |
| 149 | N-(4-(4-amino-1- methyl-7-(1-(2- (trifluorometh- yl)piperidin-4-yl)- 1H-pyrazol-4-yl)-1H- pyrazolo[4,3-c]pyridin- 3-yl)-2- ((S)-1-(4-fluoro- phenyl)ethoxy)phenyl)- 1,1-difluoro- methanesulfonamide | 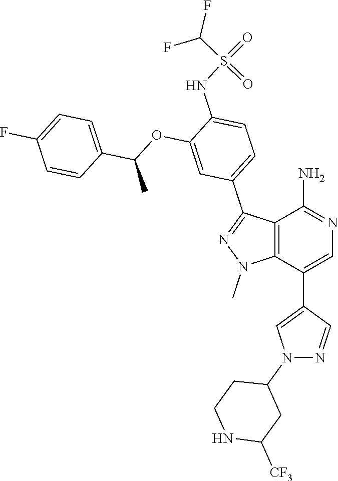 [SMILES: C[C@H](Oc1cc(-c2nn(C)c3c(-c4cnn(C5CCNC(C(F)(F)F)C5)c4)cnc(N)c23)ccc1NS(=O)(=O)C(F)F)c1ccc(F)cc1] | (Method A): 2.61 min, m/z: 709.2 [M + H]⁺ | (400 MHz, MeOD-d₄): 7.92 (s, 1H), 7.66 (s, 1H), 7.57-7.46 (m, 4H), 7.18 (dd, J = 8.1, 1.8 Hz, 1H), 7.13-7.02 (m, 3H), 6.69 (t, J = 53.3 Hz, 1H), 5.58 (q, J = 6.3 Hz, 1H), 4.48 (ddd, J = 12.0, 7.9, 4.1 Hz, 1H), 3.75 (s, 3H), 3.49 (ddd, J = 11.9, 7.0, 2.6 Hz, 1H), 3.28 (s, 1H), 2.86 (td, J = 12.8, 2.8 Hz, 1H), 2.41-2.34 (m, 1H), 2.19-2.14 (m, 1H), 2.02 (d, J = 8.1 Hz, 1H), 1.70 (d, J = 6.4 Hz, 3H) | D13 |
| 163 | N-(4-(4-amino-1- methyl-7-(1- ((3aR,6aS)- octahydrocyclo- penta[c]pyrrol-5-yl)- 1H-pyrazol-4-yl)-1H- pyrazolo[4,3-c]pyridin- 3-yl)-2-((S)-1-(4- fluoropohenyl)eth- oxy)phenyl)- 1,1-difluoromethane- sulfonamide | 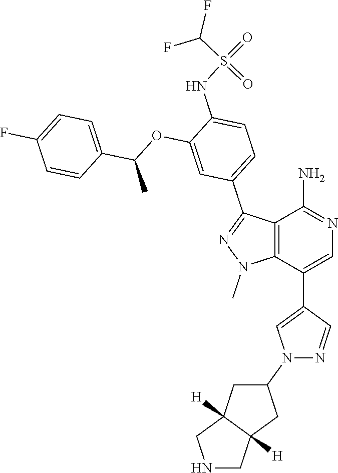 [SMILES: C[C@H](Oc1cc(-c2nn(C)c3c(-c4cnn(C5C[C@H]6CNC[C@H]6C5)c4)cnc(N)c23)ccc1NS(=O)(=O)C(F)F)c1ccc(F)cc1] | (Method A): 2.27 min, m/z: 667.2 [M + H]⁺ | (400 MHz, DMSO-d₆): 9.29 (s, 1H), 7.96 (s, 1H), 7.61 (s, 1H), 7.57-7.53 (m, 3H), 7.39 (d, J = 8 Hz, 1H), 7.18-7.04 (m, 4H), 6.86 (t, J = 52 Hz, 1H), 5.71 (s, 2H), 5.65 (q, J = 6.8 Hz, 1H), 5.00-4.97 (m, 1H), 3.69 (s, 3H), 3.00 (d, J = 8 Hz, 4H), 2.34-2.27 (m, 2H), 2.13-2.07 (m, 2H), 1.56 (d, J = 4 Hz, 3H) | D1 |

### Compound 120: N-(4-{4-amino-7-[1-(3,3-difluorocyclopentyl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

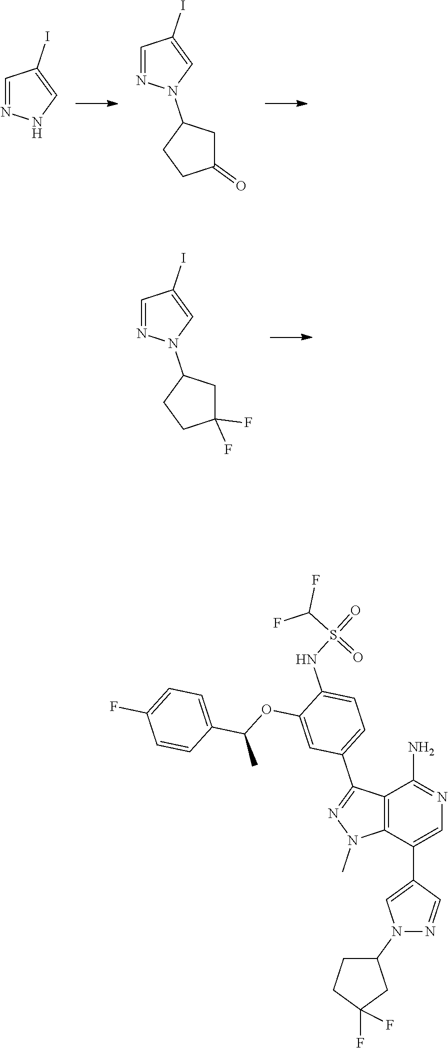 [SMILES: Ic1cn[nH]c1 ; O=C1CCC(n2cc(I)cn2)C1 ; FC1(F)CCC(n2cc(I)cn2)C1 ; C[C@H](Oc1cc(-c2nn(C)c3c(-c4cnn(C5CCC(F)(F)C5)c4)cnc(N)c23)ccc1NS(=O)(=O)C(F)F)c1ccc(F)cc1]

### Step 1: 3-(4-iodo-1H-pyrazol-1-yl)cyclopentan-1-one

To a mixture of 4-iodo-1H-pyrazole (500 mg, 2.57 mmol) and cyclopent-2-en-1-one (316 mg, 3.85 mmol) in DCM (10 mL) was added ScCl₃ (77.7 mg, 514 μmol). The reaction mixture was stirred at RT for 4 h. The mixture was concentrated under reduced pressure and the residue was purified by silica gel column chromatography (PE/EtOAc 95/5 to 20/80) to give the title product (657 mg, 93%) as a clear oil. LCMS (Method A): 3.21 min, m/z 277.0 [M+H]⁺

### Step 2: 1-(3,3-difluorocyclopentyl)-4-iodo-1H-pyrazole

To a 0° C. solution of 3-(4-iodo-1H-pyrazol-1-yl)cyclopentan-1-one (1.43 g, 5.17 mmol) in anhydrous DCM (10 mL) was added DAST (2.49 g, 15.5 mmol) in a dropwise manner, and the reaction mixture was allowed to warm to RT and stirred overnight under N₂. The reaction mixture was quenched with 1.5 M KH₂PO₄ (10 mL) and the organics were extracted 3 times with EtOAc. The combined organics were washed with brine, dried over Na₂SO₄ and concentrated under reduced pressure. The residue was purified by silica gel column chromatography (PE/EtOAc 95/5 to 20/80) to give the title product (978 mg, 63%) as a clear oil.

### Step 3: N-(4-{4-amino-7-[1-(3,3-difluorocyclopentyl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

A mixture of 1-(3,3-difluorocyclopentyl)-4-iodo-1H-pyrazole (47.9 mg, 161 μmol), N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (1 eq), Pd(OAc)_{z}(3.61 mg, 16.1 μmol), K₂CO₃ (22.2 mg, 161 μmol), Butyldi-1-adamantylphosphine (11.5 mg, 32.3 μmol) and B₂Pin₂ (40.8 mg, 161 μmol) in degassed 1,4-dioxane (4 mL) and H₂O (1 mL) was heated at 100° C. for 12 h under N₂. The solvents were evaporated, and the residue was purified by silica gel column chromatography (PE/EtOAc 80/20 to 20/80) to give the title product (5.00 mg, 9%) as a yellow solid. LCMS (Method A): 3.86 min, m/z 662.0 [M+H]⁺. ¹H NMR (400 MHz, DMSO-d₆): 8.03 (s, 1H), 7.66 (s, 1H), 7.60-7.55 (m, 2H), 7.54 (d, J=1.7 Hz, 1H), 7.40 (d, J=8.0 Hz, 1H), 7.20-7.10 (m, 4H), 7.10-6.85 (m, 1H), 5.75 (s, 2H), 5.65 (q, J=6.3 Hz, 1H), 5.00 (p, J=7.4 Hz, 1H), 2.80-2.70 (m, 1H), 2.64 (dd, J=15.7, 7.5 Hz, 1H), 2.37 (dddd, J=19.1, 9.7, 6.0, 4.0 Hz, 2H), 2.28-2.16 (m, 2H), 1.58 (d, J=6.3 Hz, 3H).

### Compound 113: N-(4-(4-amino-1-methyl-7-(1-(1,2,2-trimethylpiperidin-4-yl)-1H-pyrazol-4-yl)-1H-pyrazolo[4,3-c]pyridin-3-yl)-2-((S)-1-(4-fluorophenyl)ethoxy)phenyl)-1,1-difluoromethanesulfonamide

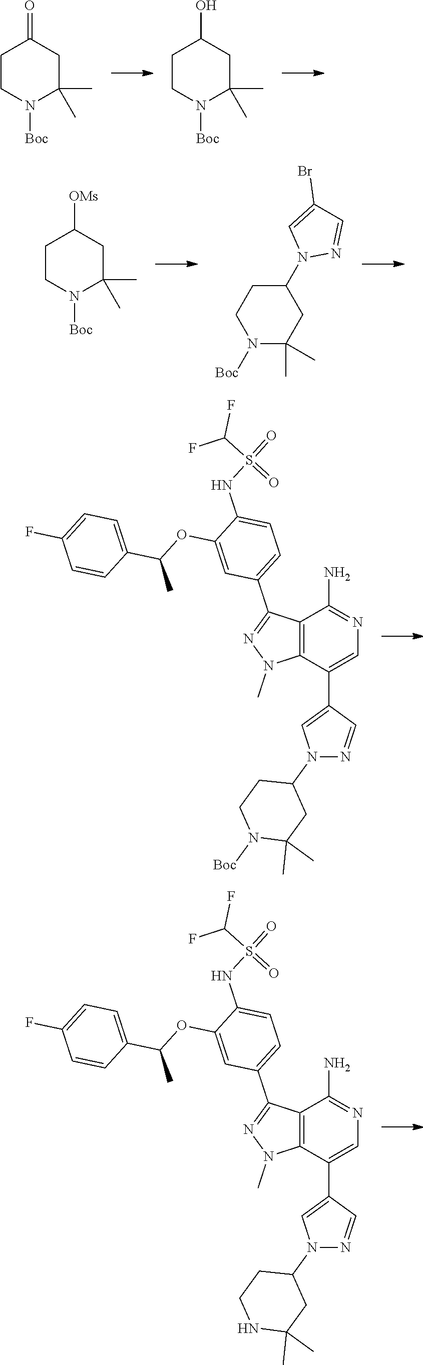

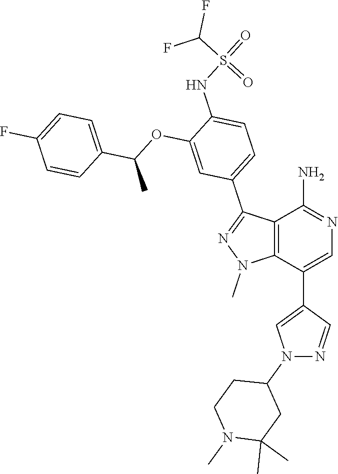 [SMILES: C[C@H](Oc1cc(-c2nn(C)c3c(-c4cnn(C5CCN(C)C(C)(C)C5)c4)cnc(N)c23)ccc1NS(=O)(=O)C(F)F)c1ccc(F)cc1]

### Step 1: tert-butyl 4-hydroxy-2,2-dimethylpiperidine-1-carboxylate

To a solution of tert-butyl 2,2-dimethyl-4-oxopiperidine-1-carboxylate (1 g, 4.39 mmol) in MeOH (20 mL) was added NaBH₄ (495 mg, 13.1 mmol). The reaction mixture was stirred at RT overnight. The reaction mixture was concentrated under reduced pressure and the residue was purified by silica gel column chromatography (PE/EtOAc: 90/10 to 10/90) to give the title product (740 mg) as yellow oil.

### Step 2: tert-butyl 4-(methanesulfonyloxy)-2,2-dimethylpiperidine-1-carboxylate

To a solution of tert-butyl 4-hydroxy-2,2-dimethylpiperidine-1-carboxylate (740 mg, 3.22 mmol) in DCM (5 mL) were added Et₃N (764 mg, 9.66 mmol) and MsCl (553 mg, 4.83 mmol). The mixture was stirred at RT for 16 h. The mixture was poured into water (20 mL) and the organics were extracted with EtOAc (3×50 mL). The combined organics were washed with brine (50 mL), dried over Na₂SO₄ and concentrated under reduced pressure to give the title product (900 mg, 2.92 mmol) as a colourless oil. Used as crude in the next step. LCMS (Method A): 3.96 min, m/z 674.4 [M+H]⁺

### Step 3: tert-butyl 4-(4-bromo-1H-pyrazol-1-yl)-2,2-dimethylpiperidine-1-carboxylate

A mixture of tert-butyl 4-(methanesulfonyloxy)-2,2-dimethylpiperidine-1-carboxylate (900 mg, 2.92 mmol), 4-bromo-1H-pyrazole (557 mg, 3.79 mmol) and Cs₂CO₃ (2.84 g, 8.76 mmol) in DMF (20 mL) was stirred at 100° C. overnight. The mixture was poured into water (50 mL) and the organics were extracted with EtOAc (50 mL×2). The combined organics were dried over Na₂SO₄ and concentrated under reduced pressure. The residue was purified by prep-TLC (PE/EtOAc; 10/1) to give the title product (420 mg, 40%) as a white solid.

### Step 4: tert-butyl 4-(4-(4-amino-3-(4-((difluoromethyl)sulfonamido)-3-((S)-1-(4-fluorophenyl)ethoxy)phenyl)-1-methyl-1H-pyrazolo[4,3-c]pyridin-7-yl)-1H-pyrazol-1-yl)-2,2-dimethylpiperidine-1-carboxylate

To a mixture of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (344 mg, 558 μmol) and tert-butyl 4-(4-bromo-1H-pyrazol-1-yl)-2,2-dimethylpiperidine-1-carboxylate (200 mg, 558 μmol) in degassed 1,4-dioxane (8 mL) and H₂O (2 mL) were added Pd(OAc)₂ (24.9 mg, 111 μmol), K₂CO₃ (153 mg, 1.11 mmol), butyldi-1-adamantylphosphine (79.9 mg, 223 μmol) and B₂pin₂ (281 mg, 1.11 mmol). The mixture was stirred at 100° C. for 12 h under N₂. The mixture was poured into water (50 mL) and the organics were extracted with EtOAc (50 mL×2). The combined organics were dried over Na₂SO₄ and concentrated under reduced pressure. The residue was purified by silica gel column chromatography (DCM/MeOH=95/5) to give the title product (170 mg, 39%) as a yellow solid. LCMS (Method A): 3.57 min, m/z 769.1 [M+H]⁺

### Step 5: N-(4-(4-amino-7-(1-(2,2-dimethylpiperidin-4-yl)-1H-pyrazol-4-yl)-1-methyl-1H-pyrazolo[4,3-c]pyridin-3-yl)-2-((S)-1-(4-fluorophenyl)ethoxy)phenyl)-1,1-difluoromethanesulfonamide

A solution of tert-butyl 4-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)-2,2-dimethylpiperidine-1-carboxylate (150 mg, 195 μmol) in HCOOH (3 mL) was stirred at RT for 4 h under N₂. The mixture was adjusted to pH=8 with the addition of sat. aq. Na₂CO₃ and concentrated under reduced pressure. The solids were washed with EtOAc (2×5 mL) and dried under reduced pressure to give the title product (120 mg, 92%) as a white solid. LCMS (Method A): 2.73 min, m/z 670.1 [M+H]⁺

### Step 6: N-(4-(4-amino-1-methyl-7-(1-(1,2,2-trimethylpiperidin-4-yl)-1H-pyrazol-4-yl)-1H-pyrazolo[4,3-c]pyridin-3-yl)-2-((S)-1-(4-fluorophenyl)ethoxy)phenyl)-1,1-difluoromethanesulfonamide

A mixture of N-(4-{4-amino-7-[1-(2,2-dimethylpiperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide (120 mg, 179.4 μmol), paraformaldehyde (72.7 mg, 897 μmol) and NaBH(OAc)₃ (190 mg, 897 μmol) in MeOH (5 mL) was stirred at RT for 45 min. The mixture was concentrated under reduced pressure and the residue was purified by silica gel column chromatography (DCM/MeOH; 95/5) to give the title product (30 mg, 25%) as a yellow solid. LCMS (Method A): 2.88 min, m/z 683.1 [M+H]⁺. ¹H NMR (400 MHz, DMSO-d₆): 7.93 (s, 1H), 7.62 (s, 1H), 7.56-7.50 (m, 2H), 7.50 (d, J=1.5 Hz, 1H), 7.36 (d, J=8.1 Hz, 1H), 7.18-7.12 (m, 2H), 7.01 (dd, J=8.1, 1.9 Hz, 1H), 6.96 (d, J=1.9 Hz, 1H), 6.65 (s, 1H), 5.66 (s, 2H), 5.64 (d, J=6.3 Hz, 1H), 4.58 (dd, J=10.9, 5.4 Hz, 1H), 3.67 (s, 3H), 3.13-3.02 (m, 3H), 2.19-2.11 (m, 2H), 2.10-2.07 (m, 1H), 2.04-1.92 (m, 1H), 1.54 (d, J=6.3 Hz, 3H), 1.29 (s, 3H), 1.24 (s, 3H).

The following compound was prepared similarly to compound 113

| Compound | Name | Structure | LCMS data | ¹H NMR data |
|---|---|---|---|---|
| 147 | N-(4-(4-amino-7-(1-(1,2- dimethylazetidin-3-yl)-1H- pyrazol-4-yl)-1-methyl-1H- pyrazolo[4,3-c]pyridin-3-yl)- 2-((S)-1-(4- fluorophenyl)ethoxy)phenyl)- 1,1- difluoromethanesulfonamide | 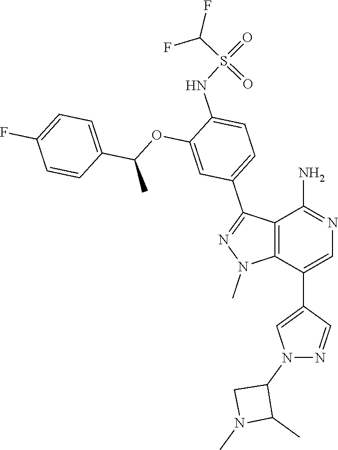 [SMILES: CC1C(n2cc(-c3cnc(N)c4c(-c5ccc(NS(=O)(=O)C(F)F)c(O[C@@H](C)c6ccc(F)cc6)c5)nn(C)c34)cn2)CN1C] | (Method A): 2.19 min, m/z: 641.2 [M + H]⁺. | (400 MHz, DMSO- d₆): 8.21 (s, 1H), 7.81 (s, 1H), 7.58-7.55 (m, 3H), 7.40 (d, J = 8 Hz, 1H), 7.19-7.08 (m, 4H), 6.98 (d, J = 52.6 Hz, 1H), 5.96 (s, 1H), 5.65 (q, J = 5.2 Hz, 1H), 4.35 (s, 1 H), 3.72 (s, 3H), 2.81 (s, 3H), 1.78-1.65 (m, 4H), 1.57 (d, J = 8 Hz, 3H), 1.54 (d, J = 8 Hz, 3H) |

### Compound 104: N-(4-{4-amino-1-methyl-7-[1-(1,6,6-trimethylpiperidin-3-yl)-1H-pyrazol-4-yl]-1H-pyrazolo[4,3-c]pyridin-3-yl}-2-[(1 S)-1-(4-fluorophenyl)ethoxy]phenyl)-1,1-difluoromethanesulfonamide

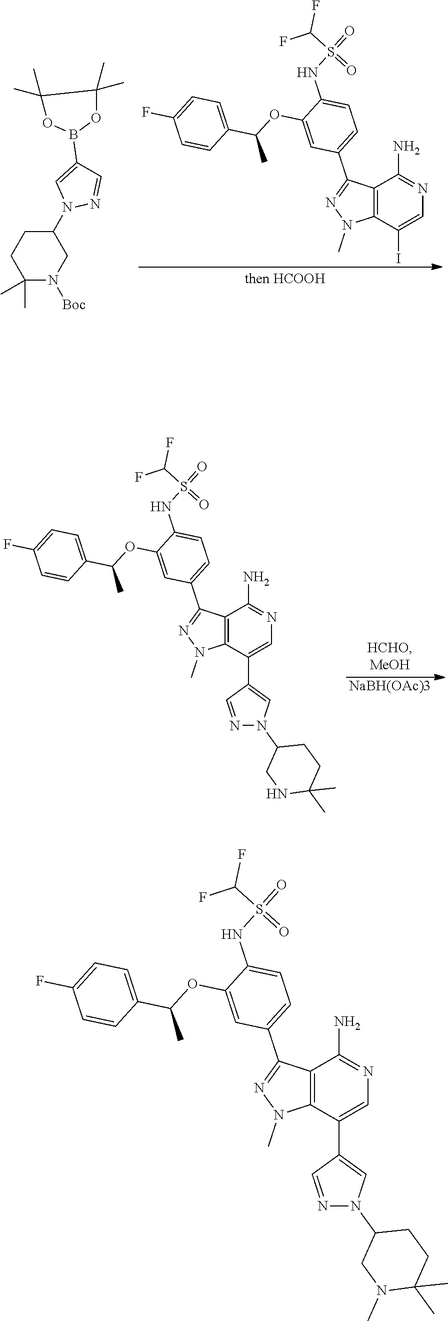

### Step 1: N-(4-{4-amino-7-[1-(6,6-dimethylpiperidin-3-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

Following General procedure A2 for the Suzuki reaction followed by acidic deprotection between intermediate A1B1 and tert-butyl 2,2-dimethyl-4-[4-(4,4,5,5-tetramethyl-1,3,2-dioxaborolan-2-yl)-1H-pyrazol-1-yl]piperidine-1-carboxylate (Intermediate C21, 100 mg, 246 μmol), the title compound (20 mg, 19%) was obtained as a colorless oil. LCMS (Method A): 2.71 min, m/z 335.2 [M+2H]²⁺

### Step 2: N-(4-{4-amino-1-methyl-7-[1-(1,6,6-trimethylpiperidin-3-yl)-1H-pyrazol-4-yl]-1H-pyrazolo[4,3-c]pyridin-3-yl}-2-[(1S)-1-(4-fluorophenyl)ethoxy]phenyl)-1,1-difluoromethanesulfonamide

A mixture of N-(4-{4-amino-7-[1-(6,6-dimethylpiperidin-3-yl)-1H-pyrazol-4-yl]-1-methyl-1H-pyrazolo[4,3-c]pyridin-3-yl-2-[(1S)-1-(4-fluorophenyl)ethoxy]phenyl)-1,1-difluoromethanesulfonamide (30 mg, 44.8 μmol), paraformaldehyde (5.43 mg, 67.2 μmol) and NaBH(OAc)₃ (47.4 mg, 224 μmol) in MeOH (5 mL) was stirred at RT for 45 min. The mixture was diluted with sat. aq. NaHCO₃ (30 mL) and DCM (30 mL). The aqueous layerwas extracted with a DCM/MeOH mixture (9/1; 3×10 mL). The combined organic layers were concentrated under reduced pressure and the residue was purified by silica gel column chromatography (DCM/MeOH 97/3) to give the title product (10.0 mg, 19%) as a yellow solid. LCMS (Method A): 2.64 min; m/z 683.1 [M+H]⁺. ¹H NMR (400 MHz, DMSO-d₆): 7.65 (d, J=3.6 Hz, 2H), 7.56 (m, 2H), 7.34-7.26 (m, 2H), 7.20 (dd, J=2.0, 8.0 Hz, 1H), 7.06-7.01 (m, 3H), 6.35 (t, J=53.6 Hz, 1H), 5.47 (q, J=2.4 Hz, 1H), 3.75 (s, 3H), 3.30-3.22 (m, 2H), 2.56 (s, 3H), 2.30-2.12 (m, 4H), 1.80-1.76 (m, 1H), 1.69 (d, J=6.4 Hz, 3H), 1.29 (s, 6H).

### Compound 111: (R)—N-(4-(4-amino-1-methyl-7-(1-(tetrahydro-2H-pyran-4-yl)-1H-pyrazol-4-yl)-1H-pyrazolo[4,3-c]pyridin-3-yl)-2-(1-(4-fluorophenyl)-2-methoxyethoxy)phenyl)-1,1-difluoromethanesulfonamide

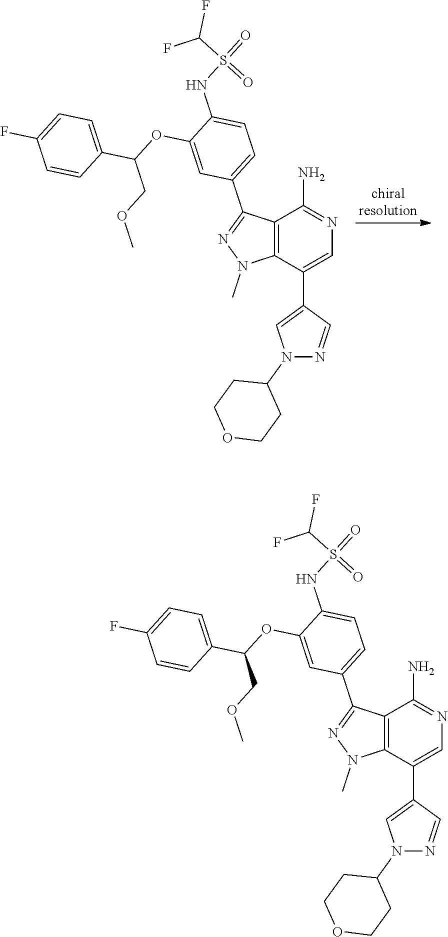 [SMILES: COCC(Oc1cc(-c2nn(C)c3c(-c4cnn(C5CCOCC5)c4)cnc(N)c23)ccc1NS(=O)(=O)C(F)F)c1ccc(F)cc1 ; COC[C@H](Oc1cc(-c2nn(C)c3c(-c4cnn(C5CCOCC5)c4)cnc(N)c23)ccc1NS(=O)(=O)C(F)F)c1ccc(F)cc1]

(R)—N-(4-(4-amino-1-methyl-7-(1-(tetrahydro-2H-pyran-4-yl)-1H-pyrazol-4-yl)-1H-pyrazolo[4,3-c]pyridin-3-yl)-2-(1-(4-fluorophenyl)-2-methoxyethoxy)phenyl)-1,1-difluoromethanesulfonamide was obtained by chiral HPLC purification of the racemic mixture (compound 102). The separation was performed on a UniChiral CND-5H (column size 21.2 mm I.D.×250 mm L) using n-Hexane/Ethanol/TFA=50/50/0.1 as the mobile phase and flow rate 25 mL/min at 25° C. Product retention time=5.38 min.

The residue was adjusted to pH=8 with sat. aq. Na₂CO₃ solution. The residue was further diluted with water and the organics were extracted with EtOAc (3 times). The combined organics were washed with brine, dried over Na₂SO₄ and concentrated under reduced pressure.

The enantiomeric excess was calculated on UniChiral CND-5H,4.6*250 mm (50% n-hexane/50% Ethanol/0.1% TFA, flow rate 1 mL/min, injection 10 μL, temperature 30° C., retention time 5.38 min) ee>98% LCMS (Method A): 3.10 min, m/z 672.1 [M+H]⁺. ¹H NMR (400 MHz, CDCl³): 7.80 (d, J=8.3 Hz, 1H), 7.58 (s, 1H), 7.56 (s, 1H), 7.41 (dd, J=8.5, 5.2 Hz, 2H), 7.28-7.27 (m, 1H), 7.12 (t, J=8.6 Hz, 2H), 6.86 (d, J=1.5 Hz, 1H), 6.40 (t, J=53.5 Hz, 1H), 4.96 (dd, J=9.6, 2.7 Hz, 1H), 4.43 (tt, J=10.2, 4.8 Hz, 1H), 4.19-4.11 (m, 2H), 3.76 (s, 3H), 3.58 (t, J=2.8 Hz, 4H), 3.55 (s, 3H), 2.17-2.09 (m, 4H).

### Compound 112: (S)—N-(4-(4-amino-1-methyl-7-(1-(tetrahydro-2H-pyran-4-yl)-1H-pyrazol-4-yl)-1H-pyrazolo[4,3-c]pyridin-3-yl)-2-(1-(4-fluorophenyl)-2-methoxyethoxy)phenyl)-1,1-difluoromethanesulfonamide

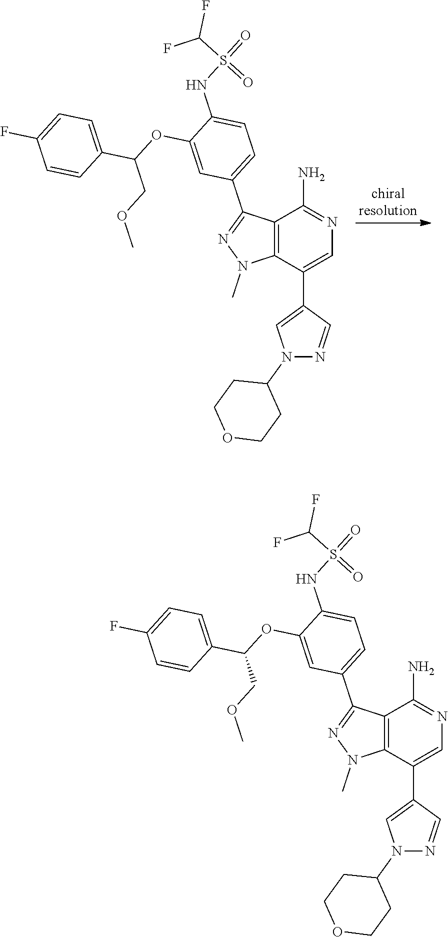 [SMILES: COCC(Oc1cc(-c2nn(C)c3c(-c4cnn(C5CCOCC5)c4)cnc(N)c23)ccc1NS(=O)(=O)C(F)F)c1ccc(F)cc1 ; COC[C@@H](Oc1cc(-c2nn(C)c3c(-c4cnn(C5CCOCC5)c4)cnc(N)c23)ccc1NS(=O)(=O)C(F)F)c1ccc(F)cc1]

(S)—N-(4-(4-amino-1-methyl-7-(1-(tetrahydro-2H-pyran-4-yl)-1H-pyrazol-4-yl)-1H-pyrazolo[4,3-c]pyridin-3-yl)-2-(1-(4-fluorophenyl)-2-methoxyethoxy)phenyl)-1,1-difluoromethanesulfonamide was obtained by chiral HPLC purification of the racemic mixture (compound 102). The separation was performed on a UniChiral CND-5H (column size 21.2 mm I.D.×250 mm L) using n-Hexane/Ethanol/TFA=50/50/0.1 as the mobile phase and flow rate 25 mL/min at 25° C. Product retention time=7.14 min.

The residue was adjusted to pH=8 with sat. aq. Na₂CO₃ solution. The residue was further diluted with water and the organics were extracted with EtOAc (3 times). The combined organics were washed with brine, dried over Na₂SO₄ and concentrated under reduced pressure.

The enantiomeric excess was calculated on UniChiral CND-5H,4.6*250 mm (50% n-hexane/50% Ethanol/0.1% TFA, flow rate 1 mL/min, injection 10 μL, temperature 30° C., retention time 5.38 min) ee>98%

LCMS (Method A): 3.12 min, m/z 672.1 [M+H]⁺. ¹H NMR (400 MHz, CDCl₃): 7.80 (d, J=8.3 Hz, 1H), 7.68 (s, 1H), 7.60 (s, 1H), 7.41 (dd, J=8.5, 5.2 Hz, 2H), 7.28-7.27 (m, 1H), 7.10 (t, J=8.6 Hz, 2H), 6.86 (d, J=1.5 Hz, 1H), 6.37 (t, J=53.5 Hz, 1H), 4.99 (dd, J=9.6, 2.7 Hz, 1H), 4.43 (tt, J=10.2, 4.8 Hz, 1H), 4.19-4.11 (m, 2H), 3.76 (s, 3H), 3.58 (t, J=2.8 Hz, 4H), 3.55 (s, 3H), 2.17-2.09 (m, 4H).

Further exemplary compounds prepared by the method similar to those described herein are detailed in Table 7 below

TABLE 7
Compounds 139, 159, 135 and 127

| Com- | | | | |
|---|---|---|---|---|
| pound | Name | Structure | LCMS data | ¹H NMR data |
| 139 | N-(4-(4-amino-1- methyl-7-(1-(1-methyl- piperidin- 4-yl)-1H-pyrazol-4-yl)- 1H-pyrazolo[4,3-c]pyridin- 3-yl)-2-(1-(4- fluorophenyl)-2- methoxyethoxy)phe- nyl)-1,1-difluoro- methanesulfonamide | 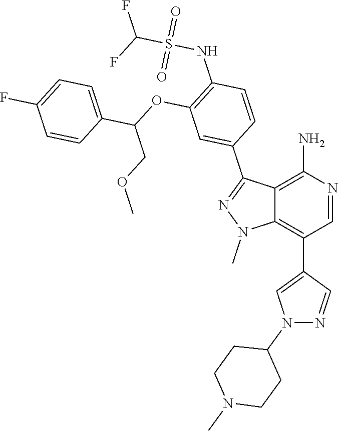 [SMILES: COCC(Oc1cc(-c2nn(C)c3c(-c4cnn(C5CCN(C)CC5)c4)cnc(N)c23)ccc1NS(=O)(=O)C(F)F)c1ccc(F)cc1] | LCMS: (Method A), 2.57 min, m/z: 685.1 [M + H]⁺ | (400 MHz, DMSO-d₆): 7.97 (s, 1H), 7.61 (s, 2H), 7.57-7.52 (m, 3H), 7.37 (d, J = 8 Hz, 1H), 7.18-7.13 (m, 2H), 7.06-7.01 (m, 2H), 6.77 (t, J = 52 Hz, 1H), 5.69 (t, J = 8 Hz, 1H), 5.62 (s, 2H), 4.29 (s, 1H), 3.81-3.77 (m, 1H), 3.67 (s, 3H), 3.62-3.58 (m, 2H), 3.17 (s, 1H), 3.13-3.10 (m, 2H), 2.45 (s, 3H), 2.12-2.07 (m, 3H). |
| 159 | N-(4-(4-amino-1- methyl-7-(1-(1,2,2- trimethylpiperidin-4- yl)-1H-pyrazol-4-yl)-1H- pyrazolo[4,3-c]pyridin- 3-yl)-2-((S)-1-(4-chloro- phenyl)ethoxy)phenyl)- 1,1-difluoro- methanesulfonamide | 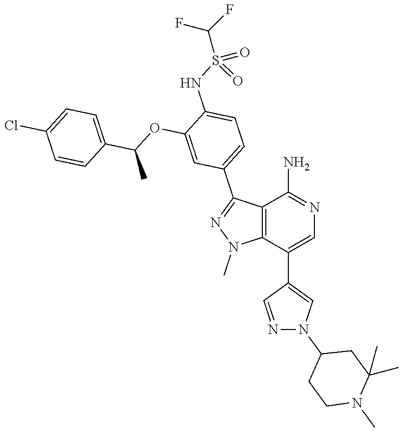 [SMILES: C[C@H](Oc1cc(-c2nn(C)c3c(-c4cnn(C5CCN(C)C(C)(C)C5)c4)cnc(N)c23)ccc1NS(=O)(=O)C(F)F)c1ccc(Cl)cc1] | LCMS: (Method A), 3.03 min, m/z 699.2 [M + H]⁺ | (400 MHz, DMSO-d₆): 7.96 (s, 1H), 7.61 (s, 1H), 7.52-7.50 (m, 3H), 7.39-7.36 (m, 3H), 7.01-6.94 (m, 2H), 6.59 (t, J = 52 Hz, 1H), 5.66-5.64 (m, 2H), 4.55 (s, 1H), 3.67 (s, 3H), 2.44 (s, 3H), 2.12-2.01 (m, 6H), 1.74-1.66 (m, 3H), 1.53 (d, J = 4 Hz, 3H), 1.25 (d, J = 12 Hz, 6H) |
| 135 | N-(4-(4-amino-1- methyl-7-(1-(tetrahydro- 2H-pyran-4-yl)-1H- pyrazol-4-yl)-1H- pyrazolo[4,3- c]pyridin-3-yl)-3-((3- fluorobenzyl)oxy)phenyl)- 1,1-difluoro- methanesulfonamide | 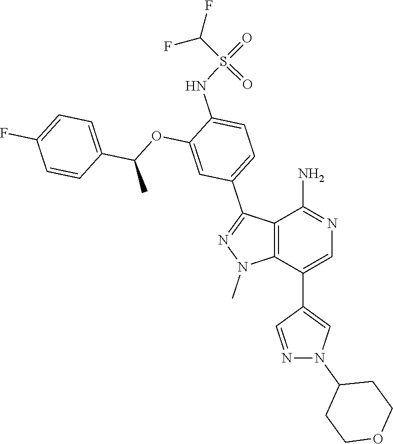 | LCMS: (Method A), 2.95 min, m/z 628.1 [M + H]⁺ | (400 MHz, DMSO-d₆): 8.05 (s, 1H), 7.64 (s, 1H), 7.53 (s, 1H), 7.32 (q, J = 7.4, 6.9 Hz, 1H), 7.26 (d, J = 8.1 Hz, 1H), 7.14 (d, J = 7.8 Hz, 1H), 7.09 (d, J = 9.7 Hz, 2H), 7.01 (s, 1H), 6.95 (s, 1H), 5.92 (s, 2H), 5.11 (s, 2H), 4.46 (dt, J = 10.5, 5.0 Hz, 1H), 3.98 (d, J = 11.0 Hz, 2H), 3.73 (s, 3H), 3.50 (d, J = 12.1 Hz, 2H), 3.46 (s, 1H), 2.02 (dt, J = 12.0, 5.5 Hz, 4H). |
| 127 | (S)-N-(4-(4-amino-1- methyl-7-(1-(tetrahydro- 2H-pyran-4-yl)-1H-pyrazol- 4-yl)-1H-pyrazolo[4,3- c]pyridin-3-yl)-2-(1-(4- fluorophenyl)eth- oxy)phenyl)-1-phenyl- methanesulfonamide | 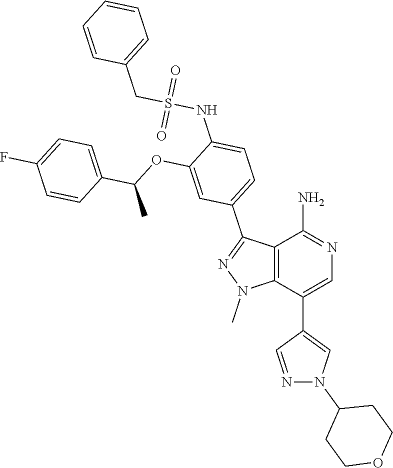 [SMILES: C[C@H](Oc1cc(-c2nn(C)c3c(-c4cnn(C5CCOCC5)c4)cnc(N)c23)ccc1NS(=O)(=O)Cc1ccccc1)c1ccc(F)cc1] | LCMS: (Method A), 3.16 min, m/z 682.1 [M + H]⁺ | (400 MHz, DMSO-d₆): 9.18 (s, 1H), 8.02 (s, 1H), 7.61-7.58 (m, 3H), 7.55 (s, 1H), 7.37-7.44 (m, 6H), 7.17 (t, J = 8.4 Hz, 2H), 7.11 (s, 1H), 7.04 (d, J = 8 Hz, 2H), 5.68-5.66 (m, 3H), 4.54 (s, 2H), 4.50-4.43 (m, 1H), 3.99-3.94 (m, 2H), 3.69 (s, 3H), 3.49 (td, J = 10.8, 2.8 Hz, 2H), 2.04-1.98 (m, 4H), 1.62 (d, J = 6 Hz, 3H) |

### General Procedure A5 for the Suzuki Reaction Between Intermediate A1B21 and Boronate Esters

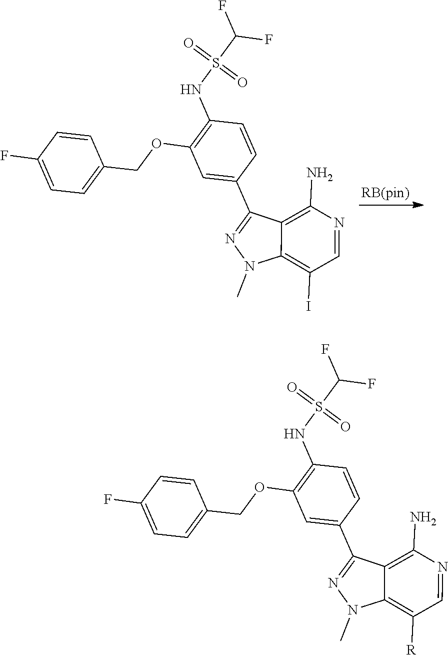

A mixture of N-(4-(4-amino-7-iodo-1-methyl-1H-pyrazolo[4,3-c]pyridin-3-yl)-2-((4-fluorobenzyl)oxy)phenyl)-1,1-difluoromethanesulfonamide (1 eq), boronate ester (1.1 eq), Na₂CO₃ (3 eq) and Pd(dppf)Cl₂ (0.1 eq) in degassed 1,4-dioxane/H₂O (4/1, 0.2 M) was stirred at 100° C. overnight under N₂. The mixture was diluted with water and the organics were extracted with EtOAc (3×). The combined organics were dried over Na₂SO₄ and concentrated under reduced pressure. The residue was purified by column chromatography (DCM/MeOH) to give the title product as a yellow solid.

The following compounds were prepared following the General procedure A5

| Com- | | | | LCMS | | |
|---|---|---|---|---|---|---|
| pound | Name | Structure | Yield | data | ¹H NMR data | Boronate ester |
| 13 | N-(4-(4-amino-1-methyl- 7-(1-(tetrahydro- 2H-pyran-4-yl)-1H- pyrazol-4-yl)-1H- pyrazolo[4,3-c]pyridin- 3-yl)-2-((4-fluoro- benzyl)oxy)phenyl)-1,1- difluoromethane- sulfonamide | 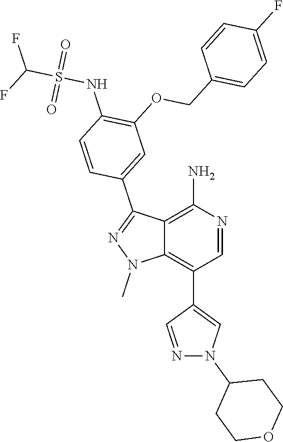 [SMILES: Cn1nc(-c2ccc(NS(=O)(=O)C(F)F)c(OCc3ccc(F)cc3)c2)c2c(N)ncc(-c3cnn(C4CCOCC4)c3)c21] | 48% | (Method A): 3.14 min, m/z: 628.2 [M + H]⁺. | (400 MHz, DMSO-d₆): 8.04 (s, 1H), 7.63-7.57 (m, 4H), 7.42 (d, J = 8 Hz, 1H), 7.36 (d, J = 1.6 Hz, 1H), 7.26-7.21 (m, 3H), 6.89 (t, J = 52.8 Hz, 1H), 5.93 (s, 2H), 5.21 (s, 2H), 4.50-4.43 (m, 1H), 3.99-3.96 (m, 3H), 3.74 (s, 3H), 2.04-1.95 (m, 4H). | 1-(oxan-4-yl)- 4-(3,3,4,4- tetramethylborolan- 1-yl)- 1H-pyrazole |
| 12 | N-(4-(4-amino-7-(1- (2-hydroxyethyl)-1H- pyrazol-4-yl)-1-methyl- 1H-pyrazolo[4,3- c]pyridin-3-yl)-2-((4- fluorobenzyl)oxy)phenyl)- 1,1-difluoro- methanesulfonamide | 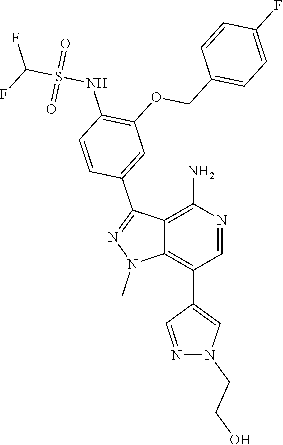 [SMILES: Cn1nc(-c2ccc(NS(=O)(=O)C(F)F)c(OCc3ccc(F)cc3)c2)c2c(N)ncc(-c3cnn(CCO)c3)c21] | 51% | (Method A): 3.07 min, m/z: 588.1 [M + H]⁺. | (400 MHz, DMSO-d₆): 7.93 (s, 1H), 7.62-7.57 (m, 4H), 7.43 (d, J = 8.0 Hz, 1H), 7.38 (d, J = 1.2 Hz, 1H), 7.27-7.22 (m, 3H), 6.92 (t, J = 52 Hz, 1H), 5.99 (s, 1H), 5.22 (s, 2H), 4.96-4.93 (m, 3H), 4.23-4.20 (m, 2H), 3.81-3.77 (m, 2H), 3.76 (s, 2H). | 2-[4-(4,4,5,5- tetramethyl- 1,3,2-dioxaborolan- 2-yl)- 1H-pyrazol-1- yl]ethan-1- ol |
| 16 | N-(4-(4-amino-1- methyl-7-(1-methyl-1H- pyrazol-4-yl)-1H- pyrazolo[4,3-c]pyridin-3- yl)-2-((4-fluoro- benzyl)oxy)phenyl)-1,1- difluoromethane- sulfonamide | 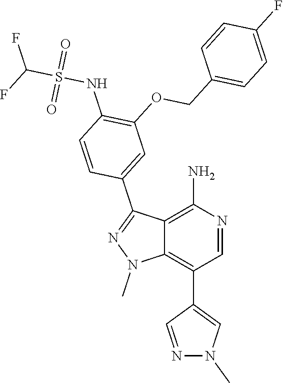 [SMILES: Cn1cc(-c2cnc(N)c3c(-c4ccc(NS(=O)(=O)C(F)F)c(OCc5ccc(F)cc5)c4)nn(C)c23)cn1] | 38% | (Method A): 3.12 min, m/z: 558.2 [M + H]⁺. | (400 MHz, DMSO-d₆): 7.93 (s, 1H), 7.87 (s, 1H), 7.59-7.52 (m, 3H), 7.49 (s, 1H), 7.35 (d, J = 8.1 Hz, 1H), 7.19 (t, J = 8.9 Hz, 2H), 7.11 (d, J = 2.0 Hz, 1H), 7.03 (dd, J = 8.1, 1.9 Hz, 1H), 6.44 (t, J = 54.1 Hz, 1H), 5.75 (s, 2H), 5.11 (s, 2H), 3.89 (s, 3H), 3.71 (s, 3H). | 1-methyl-4- (4,4,5,5- tetramethyl- 1,3,2- dioxaborolan- 2-yl)-1H- pyrazole |
| 14 | N-(4-(4-amino-1- methyl-7-(pyridin-4-yl)- 1H-pyrazolo[4,3-c]pyridin- 3-yl)-2-((4-fluoro- benzyl)oxy)phenyl)-1,1- difluoromethane- sulfonamide | 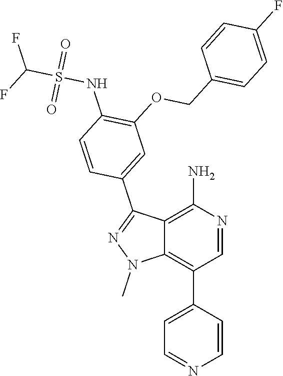 [SMILES: Cn1nc(-c2ccc(NS(=O)(=O)C(F)F)c(OCc3ccc(F)cc3)c2)c2c(N)ncc(-c3ccncc3)c21] | 38% | (Method A): 2.93 min, m/z: 554.1 [M + H]⁺. | (400 MHz, DMSO-d₆): 8.66 (d, J = 6.0 Hz, 2H), 7.68 (s, 1H), 7.63-7.59 (m, 2H), 7.55-7.53 (m, 2H), 7.44 (d, J = 8.0 Hz, 1H), 7.39 (d, J = 1.6 Hz, 1H), 7.27-7.23 (m, 3H), 6.93 (t, J = 52.8 Hz, 1H), 6.10 (s, 2H), 5.23 (s, 2H), 3.66 (s, 3H). | 4-(4,4,5,5- tetramethyl-1,3,2- dioxaborolan- 2-yl)pyridine |

### General Procedure A6 for the Suzuki Reaction Between Intermediate A1B19 and Boronate Esters/Boronic Acids

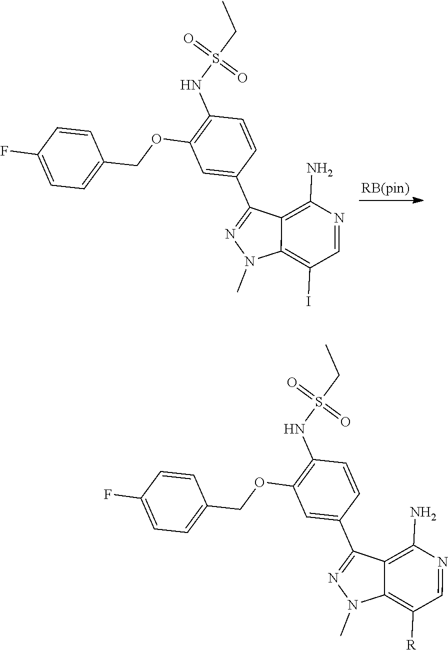

A mixture of N-(4-(4-amino-7-iodo-1-methyl-1H-pyrazolo[4,3-c]pyridin-3-yl)-2-((4-fluorobenzyl)oxy)phenyl)ethanesulfonamide (1 eq), boronate ester/boronic acid (1.1 eq), Na₂CO₃ (3 eq) and Pd(dppf)Cl₂ (0.1 eq) in degassed 1,4-dioxane/H₂O (4/1, 0.2 M) was stirred at 100° C. overnight under N₂. The mixture was diluted with water and the organics were extracted with EtOAc (3×). The combined organics were dried over Na₂SO₄ and concentrated under reduced pressure. The residue was purified by column chromatography (DCM/MeOH) to give the desired product as a yellow solid.

The following compounds were prepared following the General procedure A6

| Com- | | | | LCMS | | |
|---|---|---|---|---|---|---|
| pound | Name | Structure | Yield | data | ¹H NMR data | SM |
| 1 | N-(4-(4-amino-1- methyl-7-(pyridin- 4-yl)-1H-pyrazolo[4,3- c]pyridin-3-yl)-2-((4- fluorobenzyl)oxy)phe- nyl)ethanesulfonamide | 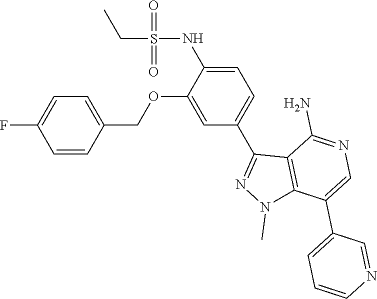 [SMILES: CCS(=O)(=O)Nc1ccc(-c2nn(C)c3c(-c4cccnc4)cnc(N)c23)cc1OCc1ccc(F)cc1] | 39% | (Method A): 2.95 min, m/z: 533.2 [M + H]⁺. | (400 MHz, CDCl₃): 9.13 (s, 1H), 8.65 (d, J = 6.0 Hz, 1H), 7.67 (s, 1H), 7.62 (q, J = 14.4 HZ, 2H), 7.52 (m, 2H), 7.47 (d, 8 Hz, 1H), 7.37 (d, J = 1.6 Hz, 1H), 7.23 (m, 3H), 6.04 (s, 2H), 5.21 (s, 2H), 3.66 (s, 3H), 3.06 (q, J = 7.4 Hz), 1.16 (t, J = 7.4 Hz). | 4-(4,4,5,5- tetramethyl- 1,3,2- dioxaborolan- 3-yl)pyridine |

### Compound 64: N-(4-(4-Aminopyrazolo[1,5-a]pyrazin-3-yl)-2-((4-fluorophenyl)methoxy) phenyl)ethane-1-sulfonamide

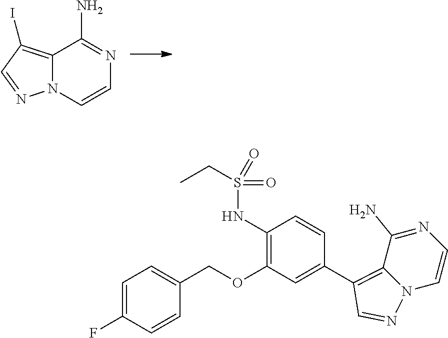 [SMILES: Nc1nccn2ncc(I)c12 ; CCS(=O)(=O)Nc1ccc(-c2cnn3ccnc(N)c23)cc1OCc1ccc(F)cc1]

### Step 1: N-(4-(4-Aminopyrazolo[1,5-a]pyrazin-3-yl)-2-((4-fluorophenyl)methoxy)phenyl) ethane-1-sulfonamide

To a mixture of 3-iodopyrazolo[1,5-a]pyrazin-4-amine (20 mg, 0.08 mmol), N-(2-((4-fluorophenyl)methoxy)-4-(4,4,5,5-tetramethyl-1,3,2-dioxaborolan-2-yl)phenyl)ethane-1-sulfonamide (36.8 mg, 0.08 mmol) and Na₂CO₃ (16.3 mg, 0.15 mmol) in 1,4-dioxane/water (4/1, 2 mL) was added Pd(dppf)Cl₂ (5.62 mg, 8 μmol), and the reaction was heated at 120° C. with μW irradiation for 1 h. The mixture was filtered over Celite, and the filtrate was concentrated under reduced pressure. The residue was purified by column chromatography (0-100% EtOAc:c-Hex) to give the title product (5.6 mg, 17%) as a brown solid. LCMS (Method D): 1.45 min, m/z 442.2 [M+H]⁺. ¹H NMR (300 MHz, CDCl₃): 7.88 (d, J=5.0 Hz, 1H), 7.84 (s, 1H), 7.66 (d, J=8.2 Hz, 1H), 7.37 (dd, J=8.7, 5.3 Hz, 2H), 7.31 (d, J=5.0 Hz, 1H), 7.13-7.07 (m, 4H), 5.12 (s, 2H), 3.15 (q, J=7.4 Hz, 2H), 1.36 (t, J=7.4 Hz, 3H).

### Compound 63: N-(4-(8-Aminoimidazo[1,5-a]pyrazin-1-yl)-2-((4-fluorophenyl)methoxy) phenyl) ethane-1-sulfonamide

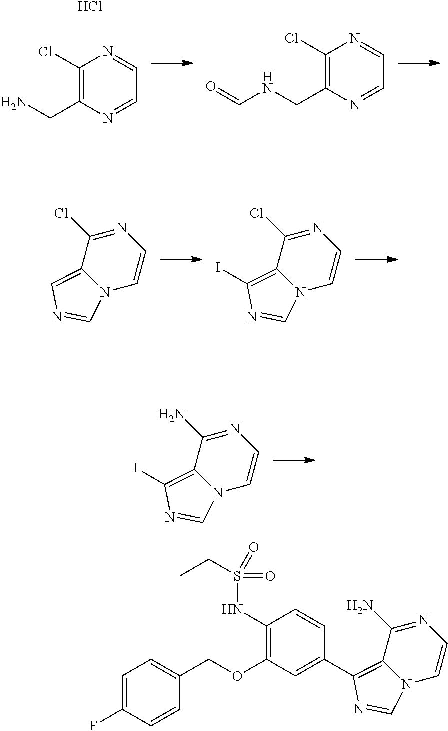

### Step 1: N-((3-Chloropyrazin-2-yl)methyl)formamide

A solution of 1-(3-chloropyrazin-2-yl)methanamine hydrochloride (1.0 g, 5.55 mmol) in HC(OMe)₃ (6.06 mL, 55.5 mmol) was heated at 110° C. under an atmosphere of N₂ overnight. The mixture was concentrated in vacuo and the residue was taken up in DCM. The organic phase was washed with water and brine, dried over MgSO₄ and concentrated in vacuo to give the title product (772 mg, 81%) as a yellow solid. LCMS (Method E): 0.33 min, m/z 172.0 [M+H]⁺. ¹H NMR (300 MHz, CDCl₃): 8.46 (d, J=2.6 Hz, 1H), 8.38 (d, J=1.0 Hz, 1H), 8.35-8.33 (m, 1H), 6.94 (bs, 1H), 4.76-4.74 (m, 2H).

### Step 2: 8-Chloroimidazo[1,5-a]pyrazine

A solution of N-((3-chloropyrazin-2-yl)methyl)formamide (0.83 g, 4.83 mmol) in MeCN (20 mL) was cooled to 0° C. before POCl₃ (1.34 mL, 14.4 mmol) was added dropwise. The reaction was stirred at 0° C. for 5 min before DMF (2 drops) was added. The reaction was warmed to RT and stirred overnight. The mixture was concentrated in vacuo and the residue was diluted with water. The pH was adjusted to ˜8 with sat. aq. NaHCO₃ and the aqueous mixture was extracted with DCM. The combined organics were dried over MgSO₄ and concentrated in vacuo to give the title product (631 mg, 85%) as a pink solid. LCMS (Method D): 0.62 min, m/z 154.2 [M+H]⁺. ¹H NMR (300 MHz, DMSO-d₆): 8.70 (d, J=0.6 Hz, 1H), 8.39 (dd, J=4.9, 1.0 Hz, 1H), 7.88 (d, J=1.0 Hz, 1H), 7.41 (d, J=4.9 Hz, 1H).

### Step 3: 8-Chloro-1-iodoimidazo[1,5-a]pyrazine

To a solution of 8-chloroimidazo[1,5-a]pyrazine (150 mg, 0.98 mmol) in DMF (20 mL) was added NIS (240 mg, 1.07 mmol) and the reaction was stirred at RT overnight. The mixture was diluted with water (40 mL) and extracted with EtOAc (2×50 mL). The combined organics were dried over MgSO₄ and concentrated in vacuo. The residue was purified by column chromatography (0-5% MeOH:DCM) to give the title product (51.9 mg, 19%) as a pale-yellow solid. LCMS (Method D): 1.15 min, m/z 280.0 [M+H]⁺. ¹H NMR (300 MHz, DMSO-d₆): 8.68 (s, 1H), 8.43 (d, J=4.9 Hz, 1H), 7.40 (d, J=4.9 Hz, 1H).

### Step 4: 1-lodoimidazo[1,5-a]pyrazin-8-amine

To a solution of 8-chloro-1-iodoimidazo[1,5-a]pyrazine (50 mg, 0.18 mmol) in MeOH (1 mL) was added 7 M NH₃ in MeOH (3 mL) and the reaction was heated at 100° C. in a sealed tube overnight. Once cooled, the mixture was concentrated in vacuo and the residue was purified by column chromatography (0-10% MeOH:DCM) to give the title product (24.9 mg, 54%) as a yellow solid. LCMS (Method D): 0.21 min, m/z 261.0 [M+H]⁺. ¹H NMR (300 MHz, DMSO-d₆): 8.35 (s, 1H), 7.23 (d, J=4.9 Hz, 1H), 6.98 (d, J=4.9 Hz, 1H), 6.58 (bs, 2H).

### Step 5: N-(4-(8-Aminoimidazo[1,5-a]pyrazin-1-yl)-2-((4-fluorophenyl)methoxy)phenyl) ethane-1-sulfonamide

To a mixture of 1-iodoimidazo[1,5-a]pyrazin-8-amine (50 mg, 0.19 mmol), N-(2-((4-fluorophenyl)methoxy)-4-(4,4,5,5-tetramethyl-1,3,2-dioxaborolan-2-yl)phenyl)ethane-1-sulfonamide (92.0 mg, 0.21 mmol) and Na₂CO₃ (40.7 mg, 0.38 mmol) in degassed 1,4-dioxane/water (4/1, 3 mL) was added Pd(dppf)Cl₂ (14.0 mg, 19.2 μmol), and the reaction was heated at 120° C. with μW irradiation for 1 h. The mixture was filtered over Celite, and the filtrate was concentrated in vacuo. The residue was purified by column chromatography (0-5% MeOH:DCM), followed by prep-HPLC (method B, gradient 30-60% MeCN) to give the title product (12.1 mg, 14%) as an orange solid (formate salt). LCMS (Method D): 1.26 min, m/z 442.2 [M+H]⁺. ¹H NMR (300 MHz, DMSO-d₆): 9.06 (s, 1H), 8.43 (s, 1H), 8.14 (s, 1H), 7.70 (d, J=4.8 Hz, 1H), 7.64-7.59 (m, 2H), 7.40 (d, J=8.1 Hz, 1H), 7.36 (s, 1H), 7.27-7.16 (m, 3H), 7.04 (d, J=4.8 Hz, 1H), 6.13 (s, 2H), 5.19 (s, 2H), 3.04 (q, J=7.3 Hz, 2H), 1.15 (t, J=7.3 Hz, 3H).

### Compound 2: N-(4-amino-3-(4-(ethylsulfonamido)-3-((4-fluorobenzyl)oxy)phenyl)-1-methyl-1H-pyrazolo[4,3-c]pyridin-7-yl)acetamide

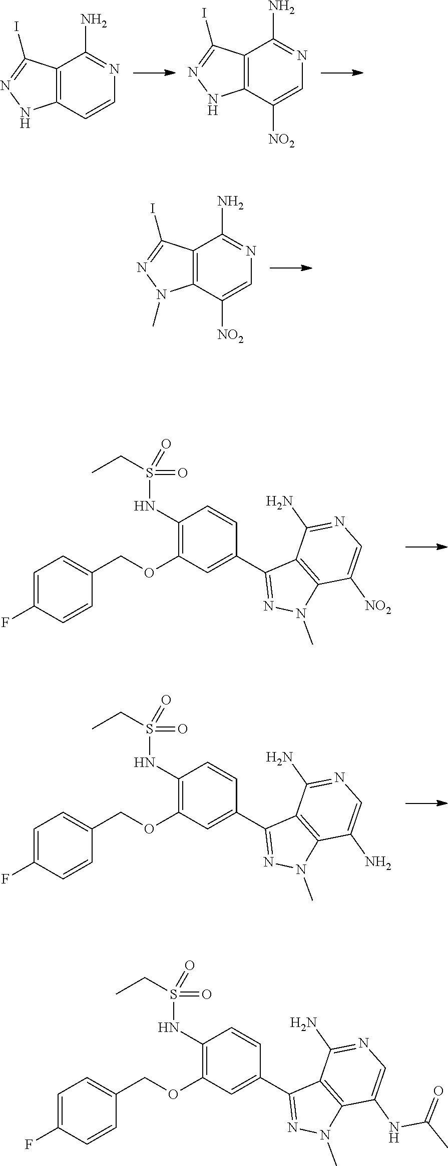 [SMILES: Nc1nccc2[nH]nc(I)c12 ; Nc1ncc([N+](=O)[O-])c2[nH]nc(I)c12 ; Cn1nc(I)c2c(N)ncc([N+](=O)[O-])c21 ; CCS(=O)(=O)Nc1ccc(-c2nn(C)c3c([N+](=O)[O-])cnc(N)c23)cc1OCc1ccc(F)cc1 ; CCS(=O)(=O)Nc1ccc(-c2nn(C)c3c(N)cnc(N)c23)cc1OCc1ccc(F)cc1 ; CCS(=O)(=O)Nc1ccc(-c2nn(C)c3c(NC(C)=O)cnc(N)c23)cc1OCc1ccc(F)cc1]

### Step 1: 3-iodo-7-nitro-1H-pyrazolo[4,3-c]pyridin-4-amine

To a solution of 3-iodo-1H-pyrazolo[4,3-c]pyridin-4-amine (500 mg, 1.92 mmol) in H₂SO₄ (50 mL) was added HNO₃ (238 μL, 5.76 mmol) and the solution was stirred at 25° C. overnight. The reaction mixture was poured into water (150 mL) and adjusted to pH=8 with an aq. NaOH solution. The mixture was extracted with EtOAc (250 mL×3) and the combined organics were washed with water and brine, dried over Na₂SO₄ and concentrated under reduced pressure to give the title product (270 mg, 46%) as a yellow solid. LCMS (method A): 2.34 min, m/z: 305.9 [M+H]⁺.

### Step 2: 3-iodo-1-methyl-7-nitro-1H-pyrazolo[4,3-c]pyridin-4-amine

A mixture of 3-iodo-7-nitro-1H-pyrazolo[4,3-c]pyridin-4-amine (160 mg, 0.52 mmol), K₂CO₃ (72.3 mg, 0.52 mmol) and iodomethane (74.4 mg, 0.52 mmol) in DMF (4 mL) was stirred at RT overnight. Water (20 mL) was added and the solids were filtered. The filter cake was washed with water (5 mL) and dried under reduced pressure to give the title product (160 mg 96%). LCMS (method A): 3.14 min, m/z: 319.9 [M+H]⁺.

### Step 3: N-(4-{4-amino-1-methyl-7-nitro-1H-pyrazolo[4,3-c]pyridin-3-yl}-2-[(4-fluorophenyl) methoxy]phenyl)ethane-1-sulfonamide

A mixture of 3-iodo-1-methyl-7-nitro-1H-pyrazolo[4,3-c]pyridin-4-amine (190 mg, 0.59 mmol), N-{2-[(4-fluorophenyl)methoxy]-4-(4,4,5,5-tetramethyl-1,3,2-dioxaborolan-2-yl)phenyl} ethane-1-sulfonamide (259 mg, 0.60 mmol), Pd(dppf)Cl₂ (24.3 mg, 0.03 mmol) and K₂CO₃ (245 mg, 1.78 mmol) in degassed 1,4-dioxane/H₂O (4/1, 2.5 mL) was stirred at 100° C. overnight. The mixture was poured into water (20 mL) and extracted with EtOAc (2×50 mL). The combined organics were dried over Na₂SO₄ and concentrated under reduced pressure. The residue was purified by column chromatography (PE/EA=1/1) to give the title product (200 mg 67%) as a yellow solid. LCMS (method A): 3.78 min, m/z: 501.1 [M+H]⁺.

### Step 4: N-(4-{4,7-diamino-1-methyl-1H-pyrazolo[4,3-c]pyridin-3-yl}-2-[(4-fluorophenyl) methoxy]phenyl)ethane-1-sulfonamide

A mixture of N-(4-{4-amino-1-methyl-7-nitro-1H-pyrazolo[4,3-c]pyridin-3-yl}-2-[(4-fluorophenyl) methoxy]phenyl)ethane-1-sulfonamide (125 mg, 0.25 mmol) and Zn dust (81.0 mg, 1.24 mmol) in MeOH (5 mL) and aqueous NH₄Cl (5 mL) was stirred at 60° C. for 2 h. The reaction mixture was poured into water (50 mL) and the organics were extracted with EtOAc (2×50 mL). The combined organics were dried over Na₂SO₄ and concentrated under reduced pressure. The residue was purified by prep-TLC (DCM/MeOH=10/1) to give the title product (27 mg, 23%) as a brown solid. LCMS (Method A): 2.97 min, m/z: 471.1 [M+H]⁺.

### Step 5: N-(4-amino-3-(4-(ethylsulfonamido)-3-((4-fluorobenzyl)oxy)phenyl)-1-methyl-1H-pyrazolo[4,3-c]pyridin-7-yl)acetamide

To a solution of N-(4-{4,7-diamino-1-methyl-1H-pyrazolo[4,3-c]pyridin-3-yl}-2-[(4-fluorophenyl)methoxy]phenyl)ethane-1-sulfonamide (27 mg, 0.057 mmol), AcOH (3.44 mg, 57 μmol) and DIEA (22.2 mg, 0.17 mmol) in DMF (1 mL) was added HATU (30.5 mg, 0.12 mmol), and the reaction was stirred at RT for 6 h. The mixture was poured into water (10 mL) and extracted with EtOAc (3×50 mL). The combined organics were dried over Na₂SO₄ and concentrated under reduced pressure. The residue was purified by prep-TLC (DCM/MeOH=20/1) to give the title product (8 mg, 27%) as a brown solid. LCMS (Method B): 2.80 min, m/z: 513.1 [M+H]⁺. ¹H NMR (400 MHz, DMSO-d₆): 9.60 (s, 1H), 9.08 (bs, 1H), 7.64-7.60 (m, 2H), 7.46-7.44 (m, 2H), 7.35 (d, J=1.6 Hz, 1H), 7.26-7.19 (m, 3H), 5.78 (s, 2H), 5.20 (s, 2H), 3.99 (s, 3H), 3.05 (q, J=7.6 Hz, 2H), 2.09 (s, 3H), 1.15 (t, J=7.2 Hz, 3H).

Similarly, the compound below was prepared using intermediate B1 (S)-1,1-difluoro-N-(2-(1-(4-fluorophenyl)ethoxy)-4-(4,4,5,5-tetramethyl-1,3,2-dioxaborolan-2-yl)phenyl)methane sulfonamide in step 3 of Compound 2 synthesis.

| Com- | | | LCMS | |
|---|---|---|---|---|
| pound | Name | Structure | data | ¹H NMR data |
| 47 | (S)-N-(4-amino-3-(4- ((difluoromethyl)sulfonamido)- 3-(1-(4-fluorophenyl)eth- oxy)phenyl)-1-methyl-1H- pyrazolo[4,3-c]pyridin-7- yl)acetamide | 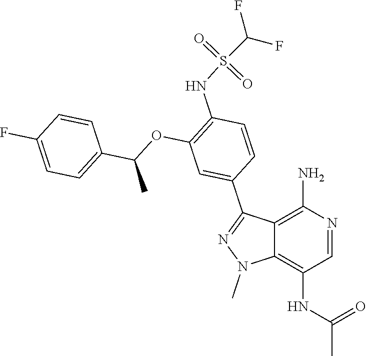 [SMILES: CC(=O)Nc1cnc(N)c2c(-c3ccc(NS(=O)(=O)C(F)F)c(O[C@@H](C)c4ccc(F)cc4)c3)nn(C)c12] | (Method A): 2.92 min, m/z: 549.2 [M + H]⁺. | (400 MHz, DMSO-d₆): 9.61 (s, 1H), 7.59-7.54 (m, 2H), 7.44 (s, 1H), 7.39 (d, J = 8.5 Hz, 1H), 7.23-7.08 (m, 5H), 6.92 (t, J = 52.7 Hz, 1H), 5.72-5.62 (m, 3H), 3.95 (s, 3H), 2.08 (s, 3H), 1.57 (d, J = 6.3 Hz, 3H). |

### Intermediate E1: N-(4-{4-amino-7-iodofuro[3,2-c]pyridin-3-yl}-2-[(1S)-1-(4-fluorophenyl) ethoxy]phenyl)-1,1-difluoromethanesulfonamide

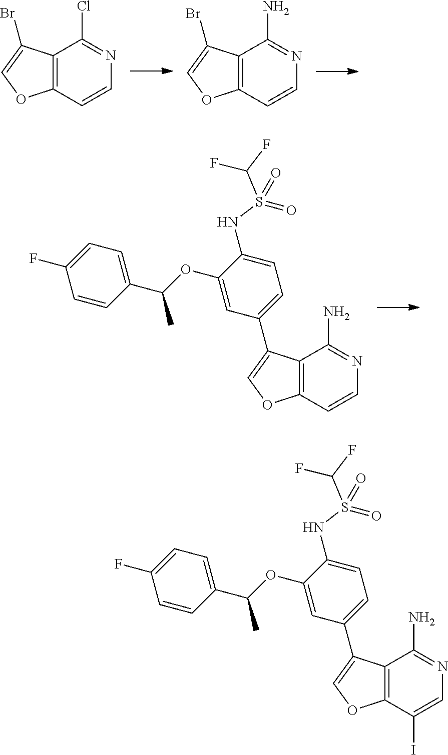

### Step 1: 3-bromofuro[3,2-c]pyridin-4-amine

A solution of 3-bromo-4-chlorofuro[3,2-c]pyridine (1 g, 4.30 mmol) in aq. NH₃ (200 mL) and 1,4-dioxane (20 mL) was stirred at 150° C. for 36 h in a sealed tube. The mixture was concentrated under reduced pressure and the residue was purified by column chromatography (PE/EA=5/1) to give the title product (400 mg, 44%) as a white solid. LCMS (Method A): 0.89 min, m/z: 212.9, 214.9 [M+H]⁺.

### Step 2: N-(4-{4-aminofuro[3,2-c]pyridin-3-yl}-2-[(1S)-1-(4-fluorophenyl)ethoxy]phenyl)-1,1-difluoromethanesulfonamide

To a solution of 3-bromofuro[3,2-c]pyridin-4-amine (350 mg, 1.64 mmol) in degassed 1,4-dioxane/H₂O (4/1, 20 mL) were added 1,1-difluoro-N-{2-[(1S)-1-(4-fluorophenyl)ethoxy]-4-(4,4,5,5-tetramethyl-1,3,2-dioxaborolan-2-yl)phenyl}methanesulfonamide (772 mg, 0.542 mmol), Na₂CO₃ (396 mg, 3.74 mmol) and Pd(dppf)Cl₂ (152 mg, 0.187 mmol). The reaction was stirred at 100° C. under N₂ overnight. The mixture was concentrated under reduced pressure and the residue was diluted with water (50 mL). The organics were extracted with EtOAc (2×50 mL), dried over Na₂SO₄ and concentrated under reduced pressure. The residue was purified by column chromatography (DCM/MeOH=50/1) to give the title product (400 mg, 51%) as a black solid. LCMS (Method A): 3.02 min, m/z: 478.1 [M+H]⁺.

### Step 3: N-(4-{4-amino-7-iodofuro[3,2-c]pyridin-3-yl}-2-[(1S)-1-(4-fluorophenyl)ethoxy]phenyl)-1,1-difluoromethanesulfonamide

To a solution of N-(4-{4-aminofuro[3,2-c]pyridin-3-yl}-2-[(1S)-1-(4-fluorophenyl)ethoxy]phenyl)-1,1-difluoromethanesulfonamide (400 mg, 0.838 mmol) in DMF (5 mL) was added NIS (375 mg, 1.67 mmol). After stirring at 85° C. for 4 h under N₂, the mixture was concentrated in vacuo. The residue was diluted with water (50 mL) and the organics were extracted with EtOAc (3×50 mL). The combined organics were washed with water and brine, dried over Na₂SO₄ and concentrated in vacuo. The residue was purified by prep-TLC (DCM/MeOH=25/1) to give the title product (90 mg, 18%) as a black solid. LCMS (Method A): 3.82 min, m/z: 604.0 [M+H]⁺.

### General Procedure A7 for the Suzuki Reaction Between Intermediate E1 and Boronate Esters

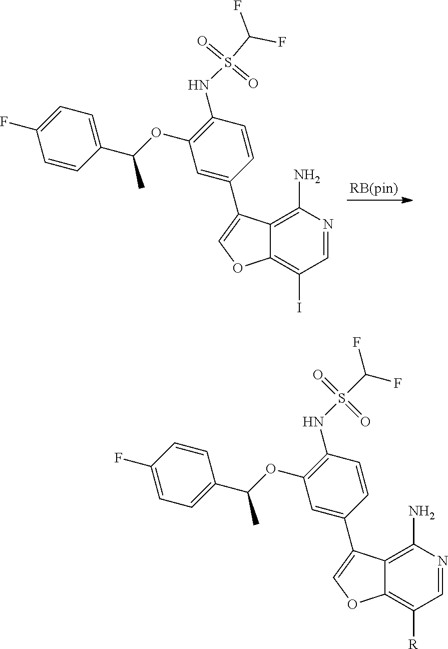

A mixture of N-(4-{4-amino-7-iodofuro[3,2-c]pyridin-3-yl}-2-[(1S)-1-(4-fluorophenyl)ethoxy]phenyl)-1,1-difluoromethanesulfonamide (1 eq), boronate ester (1.1 eq), Na₂CO₃ (3 eq) and Pd(dppf)Cl₂ (0.1 eq) in degassed 1,4-dioxane/H₂O (4/1, 0.2 M) was stirred at 100° C. overnight under N₂. The mixture was diluted with water and the organics were extracted with EtOAc (3×). The combined organics were dried over Na₂SO₄ and concentrated under reduced pressure. The residue was purified by column chromatography (DCM/MeOH) to give the title product as a yellow solid.

The following compounds were prepared following the General procedure A7

| Com- | | | | LCMS | | |
|---|---|---|---|---|---|---|
| pound | Name | Structure | Yield | data | ¹H NMR data | SM |
| 7 | (S)-N-(4-(4-amino- 7-(1-(tetrahydro-2H- pyran-4-yl)- 1H-pyrazol- 4-yl)furo[3,2- c]pyridin-3-yl)- 2-(1-(4-fluoro- phenyl)eth- oxy)phenyl)- 1,1-difluoro- methanesulfonamide | 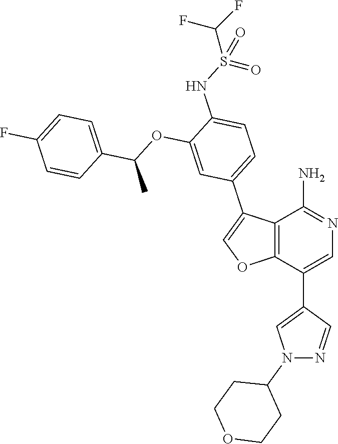 [SMILES: C[C@H](Oc1cc(-c2coc3c(-c4cnn(C5CCOCC5)c4)cnc(N)c23)ccc1NS(=O)(=O)C(F)F)c1ccc(F)cc1] | 48% | (Method A): 3.28 min, m/z: 628.2 [M + H]⁺. | (400 MHz, DMSO-d₆): 10.56 (s, 1H), 8.28 (s, 1H), 8.21 (s, 1H), 7.99 (d, J = 4.0 Hz, 2H), 7.59 (t, J = 4.0 Hz, 2H), 7.37 (d, J = 8.0 Hz, 1H), 7.17 (t, J = 8.0 Hz, 2H), 7.07 (m, 2H), 7.01 (t, J = 54.8 Hz, 1H), 5.67 (t, J = 8.0 Hz, 1H), 5.45 (s, 2H), 4.51-4.44 (m, 1H), 3.98 (d, J = 8.0 Hz, 2H), 3.48 (t, J = 8.0 Hz, 2H), 2.01 (s, 4H), 1.58 (d, J = 4.0 Hz, 3H). | 1-(oxan-4-yl)-4- (4,4,5,5- tetramethyl-1,3,2- dioxaborolan-2- yl)-1H-pyrazole |
| 9 | (S)-N-(4-(4-amino- 7-(1-(2-hydroxyethyl)- 1H-pyrazol-4- yl)furo[3,2-c]pyridin- 3-yl)-2-(1-(4-fluoro- phenyl)eth- oxy)phenyl)- 1,1-difluoromethane- sulfonamide | 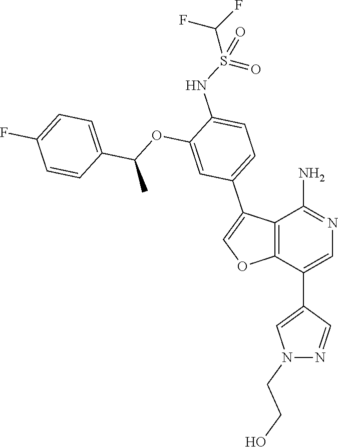 [SMILES: C[C@H](Oc1cc(-c2coc3c(-c4cnn(CCO)c4)cnc(N)c23)ccc1NS(=O)(=O)C(F)F)c1ccc(F)cc1] | 34% | (Method A): 3.07 min, m/z: 588.2 [M + H]⁺. | (400 MHz, DMSO-d₆): 8.19 (s, 2H), 7.96 (d, J = 8.0 Hz, 2H), 7.57 (t, J = 4.0 Hz, 2H), 7.36 (d, J = 4.0 Hz, 1H), 7.16 (t, J = 8.0 Hz, 2H), 7.02 (d, J = 8.0 Hz, 2H), 6.89 (t, J = 52.4 Hz, 1H), 5.67 (q, J = 4.0 Hz, 1H), 5.38 (s, 2H), 4.96 (t, J = 8.0 Hz, 1H), 4.20 (t, J = 4.0 Hz, 2H), 3.76 (q, J = 8.0 Hz, 2H), 1.57 (d, J = 8.0 Hz, 3H). | 2-[4-(4,4,5,5- tetramethyl-1,3,2- dioxaborolan-2- yl)-1H-pyrazol-1- yl]ethan-1-ol |
| 8 | (S)-N-(4-(4-amino- 7-(1-methyl-1H- pyrazol-4-yl)furo[3,2- c]pyridin-3-yl)-2-(1- (4-fluorophenyl)eth- oxy)phenyl)-1,1- difluoromethane- sulfonamide | 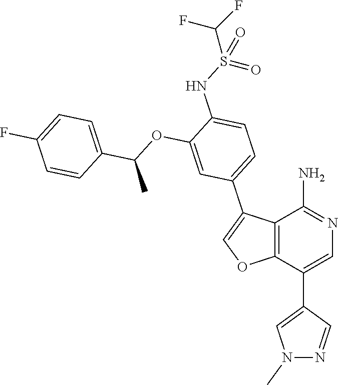 [SMILES: C[C@H](Oc1cc(-c2coc3c(-c4cnn(C)c4)cnc(N)c23)ccc1NS(=O)(=O)C(F)F)c1ccc(F)cc1] | 45% | (Method A): 3.22 min, m/z: 558.1 [M + H]⁺. | (400 MHz, DMSO-d₆): 10.48 (s, 1H), 8.18 (s, 2H), 7.96 (d, J = 4.0 Hz, 2H), 7.57 (t, J = 8.0 Hz, 2H), 7.37 (d, J = 8.0 Hz, 1H), 7.17 (t, J = 8.0 Hz, 2H), 7.06 (s, 2H), 6.97 (t, J = 52.8 Hz, 1H), 5.58 (q, J = 4.0 Hz, 1H), 5.40 (s, 2H), 3.90 (s, 3H), 1.58 (d, J = 8.0 Hz, 3H). | 1-methyl-4- (4,4,5,5- tetramethyl- 1,3,2-dioxa- borolan-2-yl)- 1H-pyrazole |

### Intermediate E2: N-(4-{4-amino-7-iodofuro[3,2-c]pyridin-3-yl}-2-[(4-fluorophenyl)methoxy]phenyl)-1,1-difluoromethanesulfonamide

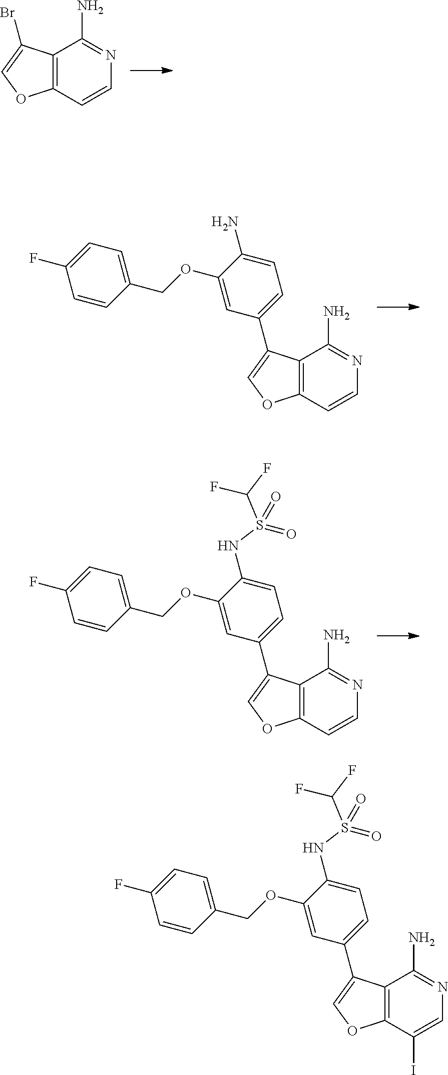

### Step 1: 3-{4-amino-3-[(4-fluorophenyl)methoxy]phenyl}furo[3,2-c]pyridin-4-amine

To a solution of 3-bromofuro[3,2-c]pyridin-4-amine (400 mg, 1.87 mmol) in degassed 1,4-dioxane/H₂O (4/1, 20 mL) were added 2-[(4-fluorophenyl)methoxy]-4-(4,4,5,5-tetramethyl-1,3,2-dioxaborolan-2-yl)aniline (641 mg, 0.5418 mmol), Na₂CO₃ (396 mg, 3.74 mmol) and Pd(dppf)Cl₂ (152 mg, 0.187 mmol). After stirring at 100° C. overnight under N₂, the mixture was concentrated under reduced pressure. The residue was diluted with water (50 mL) and extracted with EtOAc (3×50 mL). The combined organics were dried over Na₂SO₄ and concentrated under reduced pressure. The residue was purified by column chromatography (DCM/MeOH=50/1) to give the title product (460 mg, 70%) as a brown oil. LCMS (Method A): 2.89 min, m/z: 350.1 [M+H]⁺.

### Step 2: N-(4-{4-aminofuro[3,2-c]pyridin-3-yl}-2-[(4-fluorophenyl)methoxy]phenyl)-1,1-difluoromethanesulfonamide

To a solution of 3-{4-amino-3-[(4-fluorophenyl)methoxy]phenyl}furo[3,2-c]pyridin-4-amine (480 mg, 1.37 mmol) in DCM (10 mL) and pyridine (541 mg, 6.85 mmol) was added difluoromethanesulfonyl chloride (550 μL, 2.05 mmol). After stirring at RT overnight, the mixture was concentrated under reduced pressure. The residue was purified by column chromatography (DCM/MeOH=50/1) to give the title product (240 mg, 38%) as a yellow solid. LCMS (Method A): 3.22 min, m/z: 464.1 [M+H]⁺.

### Step 3: N-(4-{4-amino-7-iodofuro[3,2-c]pyridin-3-yl}-2-[(4-fluorophenyl)methoxy]phenyl)-1,1-difluoromethanesulfonamide

To a solution of N-(4-{4-aminofuro[3,2-c]pyridin-3-yl}-2-[(4-fluorophenyl)methoxy]phenyl)-1,1-difluoromethanesulfonamide (120 mg, 0.259 mmol) in DMF (3 mL) was added NIS (116 mg, 0.518 mmol). After stirring at 85° C. for 6 h under N₂, the mixture was diluted with water (50 mL) and extracted with EtOAc (3×50 mL). The combined organics were dried over Na₂SO₄ and concentrated under reduced pressure. The residue was purified by column chromatography (DCM/MeOH=25/1) to give the title product (20 mg, 13%) as a yellow solid. LCMS (Method A): 3.78 min. m/z: 590.0 [M+H]⁺.

### General Procedure A8 for the Suzuki Reaction Between Intermediate E2 and Boronate Esters

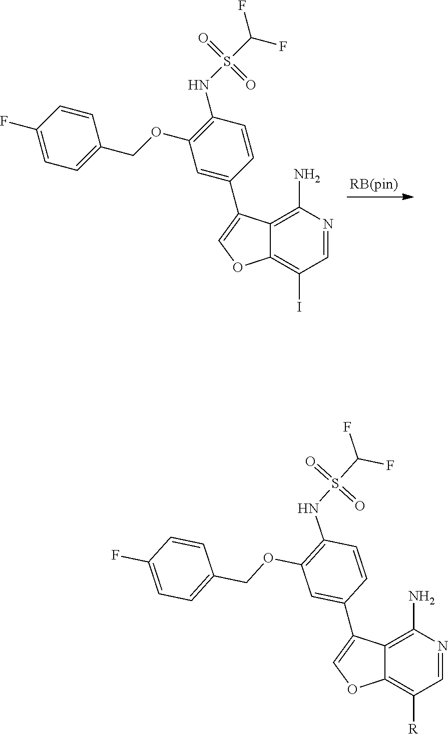

A mixture of N-(4-{4-amino-7-iodofuro[3,2-c]pyridin-3-yl}-2-[(4-fluorophenyl)methoxy]phenyl)-1,1-difluoromethanesulfonamide (1 eq), boronate ester (1.1 eq), Na₂CO₃ (3 eq) and Pd(dppf)Cl₂—CH₂Cl₂ (0.1 eq) in degassed 1,4-dioxane/H₂O (4/1, 0.2 M) was stirred at 100° C. overnight under N₂. The mixture was diluted with water and the organics were extracted with EtOAc (3×). The combined organics were dried over Na₂SO₄ and concentrated under reduced pressure. The residue was purified by column chromatography (DCM/MeOH) to give the title product as a yellow solid.

The following compounds were prepared following the General procedure A8

| Com- | | | | LCMS | | |
|---|---|---|---|---|---|---|
| pound | Name | Structure | Yield | data | ¹H NMR data | Boronic ester |
| 5 | N-(4-(4-amino-7- (1-(tetrahydro-2H- pyran-4-yl)-1H- pyrazol-4-yl)furo[3,2- c]pyridin-3-yl)-2-((4- fluorobenzyl)oxy)phenyl)- 1,1-difluoro- methanesulfonamide | 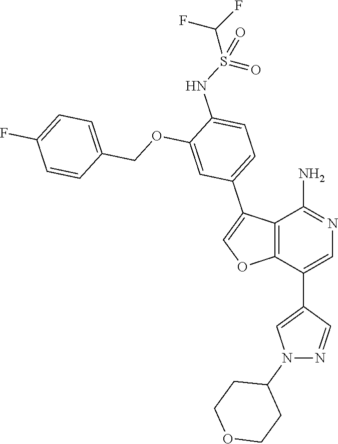 [SMILES: Nc1ncc(-c2cnn(C3CCOCC3)c2)c2occ(-c3ccc(NS(=O)(=O)C(F)F)c(OCc4ccc(F)cc4)c3)c12] | 48% | (Method A): 3.28 min, m/z: 514.2 [M + H]⁺. | (400 MHz, DMSO-d₆): 10.53 (s, 1H), 8.30 (s, 1H), 8.23 (s, 1H), 8.10 (s, 1H), 8.01 (s, 1H), 7.60 (s, 2H), 7.40 (d, J = 8.0 Hz, 1H), 7.34 (s, 2H), 7.25 (t, J = 7.2 Hz, 3H), 7.14 (d, J = 8.0 Hz, 1H), 6.90 (t, J = 52.8 Hz, 1H), 5.56 (s, 2H), 5.23 (s, 2H), 4.49 (s, 1H), 3.98 (d, J = 10.0 Hz, 2H), 2.02 (s, 4H). | 1-(tetrahydro- 2H-pyran- 4-yl)-3- (4,4,5,5- tetramethyl- 1,3,2-dioxa- borolan-2- yl)-1H- pyrazole |
| 6 | N-(4-(4-amino-7- (1-(2-hydroxyethyl)-1H- pyrazol-4-yl)furo[3,2- c]pyridin-3-yl)-2-((4- fluorobenzyl)oxy)phenyl)- 1,1-difluoro- methanesulfonamide | 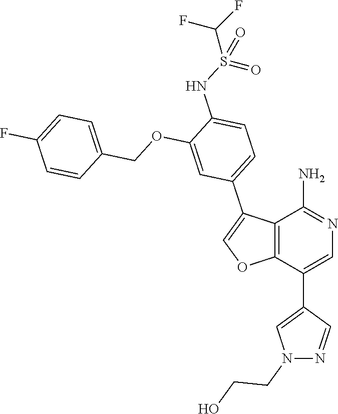 [SMILES: Nc1ncc(-c2cnn(CCO)c2)c2occ(-c3ccc(NS(=O)(=O)C(F)F)c(OCc4ccc(F)cc4)c3)c12] | 51% | (Mehod A): 3.04 min, m/z: 574.1 [M + H]⁺. | (400 MHz, DMSO-d₆): 10.55 (s, 1H), 8.23 (d, J = 2.0 Hz, 2H), 8.12 (s, 1H), 8.00 (s, 1H), 7.63-7.59 (m, 2H), 7.40 (d, J = 8.0 Hz, 1H), 7.35 (s, 1H), 7.25 (t, J = 8.8 Hz, 2H), 7.17-7.14 (m, 1H), 6.92 (t, J = 52.8 Hz, 1H), 5.57 (s, 2H), 5.23 (s, 2H), 4.94 (t, J = 5.2 Hz, 1H), 4.21 (t, J = 5.6 Hz, 2H), 3.81-3.77 (m, 2H). | 2-[4-(4,4,5,5- tetramethyl- 1,3,2-dioxa- borolan-2-yl)- 1H-pyrazol-1- yl]ethan-1-ol |

### Compound 3: N-(4-(4-amino-7-(1-(tetrahydro-2H-pyran-4-yl)-1H-pyrazol-4-yl)furo[3,2-c]pyridin-3-yl)-2-((4-fluorobenzyl)oxy)phenyl)ethanesulfonamide

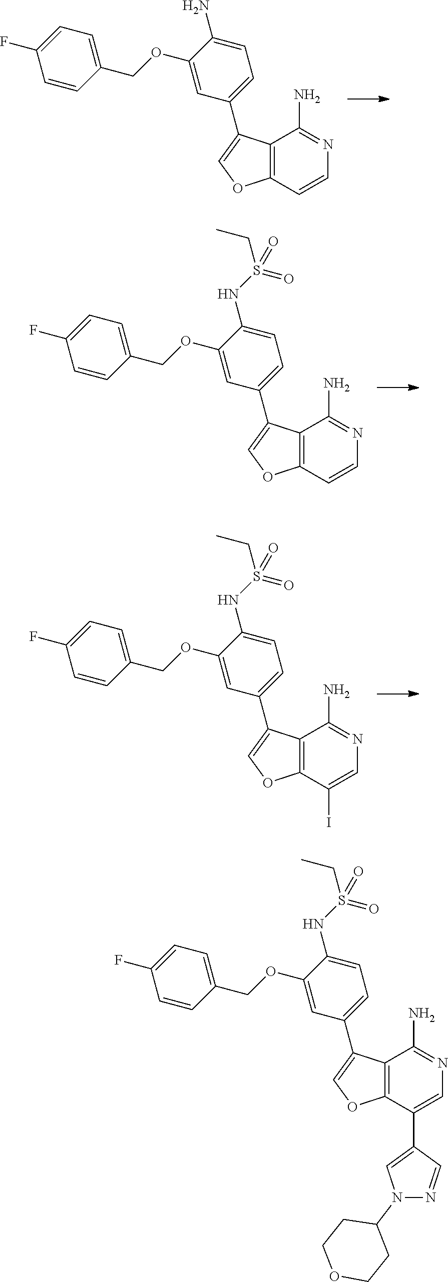

### Step 1: N-(4-{4-aminofuro[3,2-c]pyridin-3-yl}-2-[(4-fluorophenyl)methoxy]phenyl) ethane-1-sulfonamide

To a solution of 3-{4-amino-3-[(4-fluorophenyl)methoxy]phenyl}furo[3,2-c]pyridin-4-amine (420 mg, 1.20 mmol) in DCM (20 mL) and pyridine (510 μL, 5.99 mmol) was added EtSO₂Cl (230 mg, 1.79 mmol). The solution was stirred at RT overnight before being diluted with water (50 mL) and extracted with EtOAc (3×50 mL). The combined organics were washed with brine, dried over Na₂SO₄ and concentrated under reduced pressure. The residue was purified by column chromatography (DCM/MeOH=50/1) to give the title product (400 mg, 76%) as a yellow solid. LCMS (Method A): 3.04 min, m/z: 442.1 [M+H]⁺.

### Step 2: N-(4-{4-amino-7-iodofuro[3,2-c]pyridin-3-yl}-2-[(4-fluorophenyl)methoxy]phenyl) ethane-1-sulfonamide

To a solution of N-(4-{4-aminofuro[3,2-c]pyridin-3-yl}-2-[(4-fluorophenyl)methoxy]phenyl)ethane-1-sulfonamide (200 mg, 0.45 mmol) in DMF (5 mL) was added NIS (203 mg, 0.91 mmol). After stirring at 85° C. for 6 h under N₂, the mixture was concentrated under reduced pressure. The residue was diluted with water (50 mL) and the organics were extracted with EtOAc (3×50 mL). The combined organics were dried over Na₂SO₄ and concentrated under reduced pressure. The residue was purified by prep-TLC (DCM/MeOH=25/1) to give the title product (80 mg, 31%) as a yellow solid. LCMS (Method A): 3.63 min, m/z: 568.0 [M+H]⁺.

### Step 3: N-(4-(4-amino-7-(1-(tetrahydro-2H-pyran-4-yl)-1H-pyrazol-4-yl)furo[3,2-c]pyridin-3-yl)-2-((4-fluorobenzyl)oxy)phenyl)ethanesulfonamide

To a solution of N-(4-{4-amino-7-iodofuro[3,2-c]pyridin-3-yl}-2-[(4-fluorophenyl) methoxy]phenyl)ethane-1-sulfonamide (50 mg, 88 μmol) in degassed 1,4-dioxane/H₂O (4/1, 1.25 mL) were added 1-(tetrahydro-2H-pyran-4-yl)-4-(4,4,5,5-tetramethyl-1,3,2-dioxaborolan-2-yl)-1H-pyrazole (24.0 mg, 88 μmol), Pd(dppf)₂Cl₂ (7.19 mg, 8.8 μmol) and Na₂CO₃ (18.6 mg, 0.1762 mmol). After stirring at 100° C. for 1 h with μW irradiation, the mixture was concentrated under reduced pressure. The residue was diluted with water (5 mL) and the organics were extracted with EtOAc (2×5 mL). The combined organic phases were dried over Na₂SO₄ and concentrated under reduced pressure. The residue was purified by chromatography on silica gel (DCM/MeOH=20/1) to give the title product (30 mg, 58%) as a brown solid. LCMS (Method A): 3.13 min, m/z: 592.2 [M+H]⁺. ¹H NMR (400 MHz, DMSO-d₆): 9.10 (s, 1H), 8.30 (s, 1H), 8.23 (s, 1H), 8.07 (s, 1H), 8.01 (s, 1H), 7.63 (t, J=5.6 Hz, 2H), 7.43 (d, J=8.0 Hz, 1H), 7.33 (s, 1H), 7.24 (t, J=9.2 Hz, 2H), 7.12 (d, J=8.0 Hz, 1H), 5.58 (s, 2H), 5.21 (s, 2H), 4.51-4.46 (m, 1H), 3.99 (d, J=8.8 Hz, 2H), 3.52-3.46 (m, 2H), 3.02 (q, J=7.6 Hz, 2H), 2.04-1.98 (m, 4H), 1.15 (t, J=7.2 Hz, 3H).

The following compound was similarly prepared following step 3 of Compound 3

| Com- | | | | LCMS | | |
|---|---|---|---|---|---|---|
| pound | Name | Structure | Yield | data | ¹H NMR data | Boronic ester |
| 4 | N-(4-(4-amino-7- (1-(2-hydroxyethyl)-1H- pyrazol-4-yl)furo[3,2- c]pyridin-3-yl)-2-((4- fluorobenzyl)oxy)phe- nyl)ethanesulfonamide | 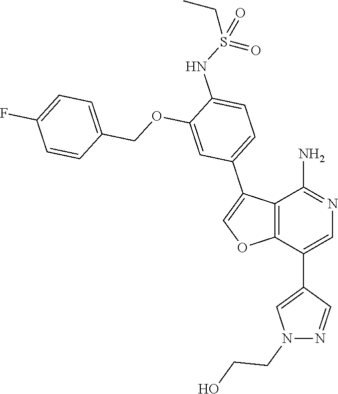 [SMILES: CCS(=O)(=O)Nc1ccc(-c2coc3c(-c4cnn(CCO)c4)cnc(N)c23)cc1OCc1ccc(F)cc1] | 69% | (Method A): 2.93 min, m/z: 552.2 [M + H]⁺. | (400 MHz, DMSO-d₆): 9.10 (s, 1H), 8.23 (s, 1H), 8.09 (s, 1H), 8.00 (s, 1H), 7.63 (t, J = 5.6 Hz, 2H), 7.43 (d, J = 8.0 Hz, 1H), 7.33 (s, 1H), 7.24 (t, J = 8.8 Hz, 2H), 7.13 (d, J = 8.0 Hz, 1H), 5.62 (s, 2H), 5.21 (s, 2H), 4.95 (t, J = 5.2 Hz, 1H), 4.22 (t, J = 5.6 Hz, 2H), 3.78 (q, J = 5.6 Hz, 2H), 3.04 (q, J = 7.2 Hz, 2H), 1.15 (t, J = 7.2 Hz, 3H). | 2-(4-(4,4,5,5- tetramethyl-1,3,2- dioxaborolan-2- yl)-1H-pyrazol-1- yl)ethan-1-ol |

### Compound 11: N-(4-(4-amino-7-(1-(tetrahydro-2H-pyran-4-yl)-1H-pyrazol-4-yl)furo[3,2-c]pyridin-3-yl)-2-((4-fluorobenzyl)oxy)phenyl)-2,2,2-trifluoroethane-1-sulfonamide

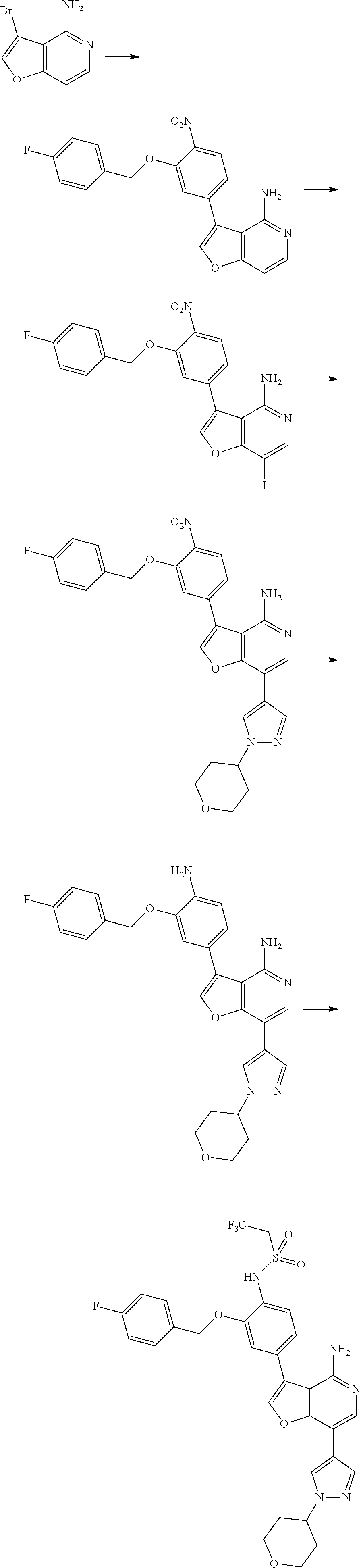 [SMILES: Nc1nccc2occ(Br)c12 ; Nc1nccc2occ(-c3ccc([N+](=O)[O-])c(OCc4ccc(F)cc4)c3)c12 ; Nc1ncc(I)c2occ(-c3ccc([N+](=O)[O-])c(OCc4ccc(F)cc4)c3)c12 ; Nc1ncc(-c2cnn(C3CCOCC3)c2)c2occ(-c3ccc([N+](=O)[O-])c(OCc4ccc(F)cc4)c3)c12 ; Nc1ccc(-c2coc3c(-c4cnn(C5CCOCC5)c4)cnc(N)c23)cc1OCc1ccc(F)cc1 ; Nc1ncc(-c2cnn(C3CCOCC3)c2)c2occ(-c3ccc(NS(=O)(=O)CC(F)(F)F)c(OCc4ccc(F)cc4)c3)c12]

### Step 1: 3-{3-[(4-fluorophenyl)methoxy]-4-nitrophenyl}furo[3,2-c]pyridin-4-amine

To a solution of 3-bromofuro[3,2-c]pyridin-4-amine (570 mg, 2.67 mmol) in degassed 1,4-dioxane/H₂O (4/1, 30 mL) were added 2-{3-[(4-fluorophenyl)methoxy]-4-nitrophenyl}-4,4,5,5-tetramethyl-1,3,2-dioxaborolane (996 mg 2.67 mmol), Na₂CO₃ (396 mg, 3.74 mmol) and Pd(dppf)Cl₂ (152 mg, 0.19 mmol). After stirring at 100° C. overnight under N₂, the mixture was concentrated under reduced pressure and the residue was purified by column chromatography (DCM/MeOH=100/1) to give the title product (610 mg, 60%) as a yellow solid. LCMS (Method A): 3.13 min, m/z: 380.1 [M+H]⁺.

### Step 2: 3-{3-[(4-fluorophenyl)methoxy]-4-nitrophenyl}-7-iodofuro[3,2-c]pyridin-4-amine

To a solution of 3-{3-[(4-fluorophenyl)methoxy]-4-nitrophenyl}furo[3,2-c]pyridin-4-amine (550 mg, 1.44 mmol) in DMF (11 mL) was added NIS (647 mg, 2.88 mmol). After stirring at 85° C. for 4 h under N₂, the reaction mixture was concentrated under reduced pressure. The residue was diluted with water (50 mL) and the organics were extracted with EtOAc (3×50 mL). The combined organic phases were dried over Na₂SO₄ and concentrated under reduced pressure. The residue was purified by column chromatography (PE/EA=1/1) to give the title product (530 mg, 73%) as a brown solid. LCMS (Method A): 4.01 min, m/z: 506.0 [M+H]⁺.

### Step 3: 3-{3-[(4-fluorophenyl)methoxy]-4-nitrophenyl}-7-[1-(oxan-4-yl)-1H-pyrazol-4-yl]furo[3,2-c]pyridin-4-amine

To a solution of 3-{3-[(4-fluorophenyl)methoxy]-4-nitrophenyl}-7-iodofuro[3,2-c]pyridin-4-amine (200 mg, 396 μmol) in degassed 1,4-dioxane/H₂O (4/1, 5 mL) were added 1-(oxan-4-yl)-4-(4,4,5,5-tetramethyl-1,3,2-dioxaborolan-2-yl)-1H-pyrazole (110 mg, 396 μmol), Pd(dppf)Cl₂ (32.3 mg, 39.6 μmol) and Na₂CO₃ (83.9 mg, 0.79 mmol). After stirring at 100° C. for 2 h with μW irradiation, the mixture was concentrated under reduced pressure. The residue was diluted with water (5 mL) and the organics were extracted with EtOAc (3×5 mL). The combined organic phases were dried over Na₂SO₄ and concentrated under reduced pressure. The residue was purified by column chromatography (DCM/MeOH=20/1) to give the title product (45 mg, 22%) as a yellow solid. LCMS (Method A): 3.40 min, m/z: 530.2 [M+H]⁺.

### Step 4: 3-{4-amino-3-[(4-fluorophenyl)methoxy]phenyl}-7-[1-(oxan-4-yl)-1H-pyrazol-4-yl]furo[3,2-c]pyridin-4-amine

To a solution of 3-{3-[(4-fluorophenyl)methoxy]-4-nitrophenyl}-7-[1-(oxan-4-yl)-1H-pyrazol-4-yl]furo[3,2-c]pyridin-4-amine (45 mg, 0.085 mmol) in MeOH (6 mL) was added sat. aq. NH₄Cl (2 mL) followed by Zn dust (27.7 mg, 0.425 mmol). After stirring at 60° C. overnight, the reaction mixture was concentrated under reduced pressure. The residue was diluted with water (5 mL) and extracted with EtOAc (3×5 mL). The combined organic phases were dried over Na₂SO₄ and concentrated under reduced pressure. The residue was purified by column chromatography (DCM/MeOH=20/1) to give the title product (40 mg, 94%) as a yellow solid. LCMS (Method A): 3.16 min, m/z: 500.2 [M+H]⁺.

### Step 5: N-(4-(4-amino-7-(1-(tetrahydro-2H-pyran-4-yl)-1H-pyrazol-4-yl)furo[3,2-c]pyridin-3-yl)-2-((4-fluorobenzyl)oxy)phenyl)-2,2,2-trifluoroethane-1-sulfonamide

To a solution of 3-{4-amino-3-[(4-fluorophenyl)methoxy]phenyl}-7-[1-(oxan-4-yl)-1H-pyrazol-4-yl]furo[3,2-c]pyridin-4-amine (50 mg, 0.1 mmol) in DCM (5 mL) and pyridine (40.2 μL, 0.5 mmol) was added 2,2,2-trifluoroethane-1-sulfonyl chloride (18.2 mg, 0.1 mmol). After stirring at RT overnight, the mixture was concentrated under reduced pressure and the residue was purified by column chromatography (DCM/MeOH=20/1) to give the title product (30 mg, 47%) as a yellow solid. LCMS (Method A): 3.32 min, m/z: 646.2 [M+H]⁺. ¹H NMR (400 MHz, DMSO-d₆): 9.92 (s, 1H), 8.29 (s, 1H), 8.22 (s, 1H), 8.09 (s, 1H), 8.01 (s, 1H), 7.60 (t, J=8.0 Hz, 2H), 7.40 (d, J=1.0 Hz, 1H), 7.34 (s, 1H), 7.23 (t, J=8.0 Hz, 2H), 7.15 (d, J=8.0 Hz, 1H), 5.59 (s, 2H), 5.21 (s, 2H), 4.51-4.44 (m, 1H), 4.29 (q, J=9.6 Hz, 2H), 3.98 (d, J=11.2 Hz, 4H), 2.01 (s, 4H).

### Compound 62: N-(4-(3-Amino-1H-indazol-4-yl)-2-((4-fluorophenyl)methoxy)phenyl)ethane-1-sulfonamide

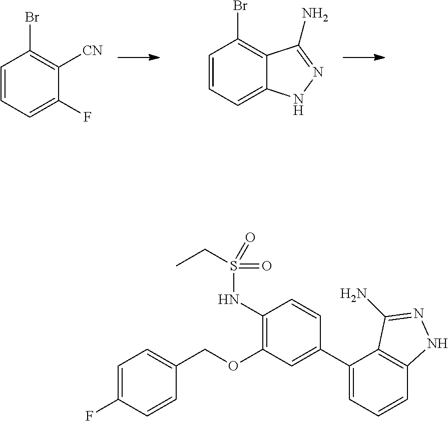 [SMILES: N#Cc1c(F)cccc1Br ; Nc1n[nH]c2cccc(Br)c12 ; CCS(=O)(=O)Nc1ccc(-c2cccc3[nH]nc(N)c23)cc1OCc1ccc(F)cc1]

### Step 1: 4-Bromo-1H-indazol-3-amine

To a solution of 2-bromo-6-fluorobenzonitrile (200 mg, 1.00 mmol) in EtOH (2 mL) was added NH₂NH₂—H₂O (241 μL, 4.99 mmol) and the reaction was heated at reflux for 12 h. Water was added, and the mixture was chilled on ice. The precipitate was collected via filtration and air-dried to give the title product (195 mg, 92%) as a white solid. LCMS (Method D): 1.07 min, m/z 214.0 [M+H]⁺. ¹H NMR (300 MHz, DMSO-d₆): 7.26 (dd, J=7.5, 1.6 Hz, 1H), 7.13-7.05 (m, 2H), 5.13 (s, 2H).

### Step 2: N-(4-(3-Amino-1H-indazol-4-yl)-2-((4-fluorophenyl)methoxy)phenyl)ethane-1-sulfonamide

To a mixture of 4-bromo-1H-indazol-3-amine (50 mg, 0.24 mmol), N-(2-((4-fluorophenyl) methoxy)-4-(4,4,5,5-tetramethyl-1,3,2-dioxaborolan-2-yl)phenyl)ethane-1-sulfonamide (112 mg, 0.26 mmol) and Na₂CO₃ (49.9 mg, 0.47 mmol) in 1,4-dioxane/H₂O (4/1, 5 mL) was added Pd(dppf)Cl₂ (17.2 mg, 0.02 mmol), and the reaction was heated at 100° C. with μW irradiation for 30 min. The mixture was filtered over Celite, and the filtrate was concentrated in vacuo. The residue was purified by column chromatography (0-5% MeOH:DCM) to give the title compound (89.9 mg, 87%) as a brown solid. LCMS (Method D): 1.88 min, m/z 441.2 [M+H]⁺. ¹H NMR (300 MHz, DMSO-d₆): 9.06 (s, 1H), 7.61 (dd, J=8.8, 5.6 Hz, 2H), 7.40 (d, J=8.1 Hz, 1H), 7.21-7.29 (m, 5H), 7.04 (dd, J=8.1, 1.8 Hz, 1H), 6.82 (dd, J=3.8, 3.8 Hz, 1H), 5.21 (s, 2H), 4.34 (s, 2H), 3.04 (q, J=7.3 Hz, 2H), 1.16 (t, J=7.3 Hz, 3H).

### Compound 67: N-(4-(3-Amino-1H-pyrazolo[3,4-b]pyridin-4-yl)-2-((4-fluorophenyl)methoxy) phenyl)-2,2,2-trifluoroethane-1-sulfonamide

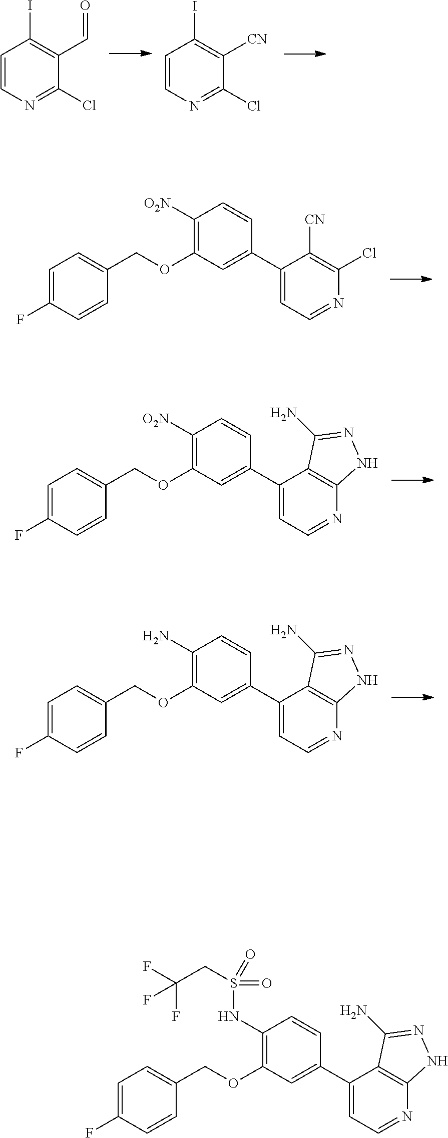 [SMILES: O=Cc1c(I)ccnc1Cl ; N#Cc1c(I)ccnc1Cl ; N#Cc1c(-c2ccc([N+](=O)[O-])c(OCc3ccc(F)cc3)c2)ccnc1Cl ; Nc1n[nH]c2nccc(-c3ccc([N+](=O)[O-])c(OCc4ccc(F)cc4)c3)c12 ; Nc1ccc(-c2ccnc3[nH]nc(N)c23)cc1OCc1ccc(F)cc1 ; Nc1n[nH]c2nccc(-c3ccc(NS(=O)(=O)CC(F)(F)F)c(OCc4ccc(F)cc4)c3)c12]

### Step 1: 2-Chloro-4-iodopyridine-3-carbonitrile

To a solution of 2-chloro-4-iodopyridine-3-carbaldehyde (1.0 g, 3.73 mmol) in THE (11 mL) were added 25% NH₄OH (11 mL, 69.9 mmol) and 12 (1.04 g, 4.10 mmol), and the reaction was stirred at RT for 18 h. The reaction was quenched by the addition of sat. NaHSO₃ and extracted with EtOAc. The organic phase was washed with brine, dried over MgSO₄ and concentrated in vacuo to give the title compound (970 mg, 98%) as a yellow solid. LCMS (Method D): 1.54 min, m/z 264.8 [M+H]⁺. ¹H NMR (300 MHz, CDCl₃): 8.18 (d, J=5.3 Hz, 1H), 7.83 (d, J=5.3 Hz, 1H).

### Step 2: 2-Chloro-4-(3-((4-fluorophenyl)methoxy)-4-nitrophenyl)pyridine-3-carbonitrile

To a mixture of 2-chloro-4-iodopyridine-3-carbonitrile (970 mg, 3.66 mmol), 2-(3-((4-fluorophenyl)methoxy)-4-nitrophenyl)-4,4,5,5-tetramethyl-1,3,2-dioxaborolane (1.50 g, 4.02 mmol) and Na₂CO₃ (775 mg, 7.32 mmol) in 1,4-dioxane/water (9/1, 37 mL) was added Pd(dppf)Cl₂—CH₂Cl₂ (298 mg, 0.37 mmol), and the reaction was heated at 100° C. under N₂ for 20 h. Once cooled, the mixture was concentrated in vacuo and the residue was partitioned between EtOAc (50 mL) and water (50 mL). The phases were separated, and the aqueous phase was extracted with EtOAc (3×50 mL). The combined organics were washed with brine, dried over MgSO₄ and concentrated in vacuo. The residue was purified by column chromatography (0-50% EtOAc: n-Hep) to give the title compound (1.08 g, 77%) as a yellow solid. LCMS (Method D): 2.52 min, molecular ion not observed. ¹H NMR (300 MHz, DMSO-d₆): 8.82 (d, J=5.2 Hz, 1H), 8.12 (d, J=8.3 Hz, 1H), 7.83 (d, J=1.6 Hz, 1H), 7.80 (d, J=5.2 Hz, 1H), 7.54-7.48 (m, 2H), 7.46 (d, J=1.6 Hz, 1H), 7.26 (dd, J=8.9 Hz, 2H), 5.34 (s, 2H).

### Step 3: 4-(3-((4-Fluorophenyl)methoxy)-4-nitrophenyl)-1H-pyrazolo[3,4-b]pyridin-3-amine

To a solution of 2-chloro-4-(3-((4-fluorophenyl)methoxy)-4-nitrophenyl)pyridine-3-carbonitrile (1.05 g, 2.73 mmol) in i-PrOH (27 mL) was added NH₂NH₂—H₂O (264 μL, 5.46 mmol) and the reaction was heated at 80° C. with μW irradiation for 1 h. Once cooled, the precipitate was collected via filtration to give the title product (878 mg, 85%) as an orange solid. LCMS (Method D): 1.97 min, m/z 380.0 [M+H]⁺. ¹H NMR (300 MHz, DMSO-d₆): 12.45 (bs, 1H), 8.46 (d, J=4.7 Hz, 1H), 8.06 (d, J=8.3 Hz, 1H), 7.67 (d, J=1.6 Hz, 1H), 7.52 (dd, J=8.8, 5.5 Hz, 2H), 7.35 (dd, J=8.3, 1.6 Hz, 1H), 7.26 (dd, J=8.8 Hz, 2H), 7.03 (d, J=4.7 Hz, 1H), 5.39 (s, 2H), 4.76 (s, 2H).

### Step 4: 4-(4-Amino-3-((4-fluorophenyl)methoxy)phenyl)-1H-pyrazolo[3,4-b]pyridin-3-amine

To a solution of 4-(3-((4-fluorophenyl)methoxy)-4-nitrophenyl)-1H-pyrazolo[3,4-b]pyridin-3-amine (878 mg, 2.31 mmol) in EtOH:THF:H₂O (4:4:1, 36 mL) were added Fe (642 mg, 11.5 mmol) and NH₄Cl (123 mg, 2.31 mmol), and the reaction was heated at reflux overnight. Whilst hot, the mixture was filtered over Celite and the pad was rinsed with EtOH. The combined filtrates were concentrated in vacuo and the residue was purified by column chromatography (0-20% MeOH:DCM) to give the title product (445 mg, 55%) as a yellow glass. LCMS (Method D): 1.50 min, m/z 350.2 [M+H]⁺. ¹H NMR (300 MHz, DMSO-d₆): 12.16 (s, 1H), 8.29 (d, J=4.8 Hz, 1H), 7.57 (dd, J=8.8, 5.6 Hz, 2H), 7.72 (dd, J=8.8 Hz, 2H), 7.16 (d, J=1.8 Hz, 1H), 6.99 (dd, J=8.0, 1.8 Hz, 1H), 6.84 (d, J=4.8 Hz, 1H), 6.81 (d, J=8.0 Hz, 1H), 5.17 (s, 2H), 4.61 (s, 2H).

### Step 5: N-(4-(3-Amino-1H-pyrazolo[3,4-b]pyridin-4-yl)-2-((4-fluorophenyl)methoxy) phenyl)-2,2,2-trifluoroethane-1-sulfonamide

To a solution of 4-(4-amino-3-((4-fluorophenyl)methoxy)phenyl)-1H-pyrazolo[3,4-b]pyridin-3-amine (103.5 mg, 0.29 mmol) in CHCl₃ (3 mL) were added pyridine (71.1 μL, 0.88 mmol) and 2,2,2-trifluoroethanesulfonyl chloride (80.7 mg, 0.44 mmol), and the reaction was stirred under N₂ overnight. The mixture was concentrated in vacuo and the residue was purified by column chromatography (0-10% MeOH:DCM) followed by prep-HPLC purification (method B, gradient 30-60% MeCN) to give the title compound (13.3 mg, 9%) as a yellow solid. LCMS (Method D): 1.93 min, m/z 496.0 [M+H]⁺. ¹H NMR (300 MHz, DMSO-d₆): 12.33 (s, 1H), 9.94 (s, 1H), 8.40 (d, J=4.7 Hz, 1H), 7.65-7.55 (m, 2H), 7.44 (d, J=8.1 Hz, 1H), 7.40 (d, J=1.8 Hz, 1H), 7.29-7.15 (m, 3H), 6.95 (d, J=4.7 Hz, 1H), 5.24 (s, 2H), 4.67 (s, 2H), 4.33 (q, J=9.8 Hz, 2H).

### Compound 66: N-(4-(3-Amino-1H-pyrazolo[3,4-b]pyridin-4-yl)-2-((4-fluorophenyl)methoxy) phenyl)-1,1-difluoromethanesulfonamide

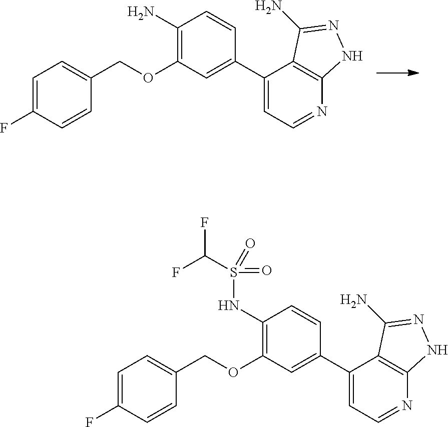 [SMILES: Nc1ccc(-c2ccnc3[nH]nc(N)c23)cc1OCc1ccc(F)cc1 ; Nc1n[nH]c2nccc(-c3ccc(NS(=O)(=O)C(F)F)c(OCc4ccc(F)cc4)c3)c12]

### Step 1: N-(4-(3-Amino-1H-pyrazolo[3,4-b]pyridin-4-yl)-2-((4-fluorophenyl)methoxy) phenyl)-1,1-difluoromethanesulfonamide

To a solution of 4-(4-amino-3-((4-fluorophenyl)methoxy)phenyl)-1H-pyrazolo[3,4-b]pyridin-3-amine (90 mg, 0.26 mmol) in pyridine (2.5 mL) was added difluoromethanesulfonyl chloride (45.6 μL, 0.52 mmol), and the reaction was stirred at RT under N₂ overnight. The mixture was concentrated in vacuo and the residue was azeotropically distilled with MePh. The residue was purified by column chromatography (0-20% MeOH:DCM) followed by prep-HPLC purification (Method B, gradient 30-60% MeCN) to give the title product (3.50 mg, 3%) as a yellow solid. LCMS (Method D): 1.78 min, m/z 464.0 [M+H]⁺. ¹H NMR (300 MHz, DMSO-d₆): 10.54 (s, 1H), 8.38 (d, J=4.8 Hz, 1H), 7.59 (dd, J=8.6, 5.6 Hz, 2H), 7.41 (d, J=8.1 Hz, 1H), 7.33 (s, 1H), 7.23 (t, J=8.9 Hz, 2H), 7.15 (d, J=7.6 Hz, 1H), 6.93 (d, J=4.8 Hz, 1H), 5.22 (s, 2H), 4.62 (s, 2H).

### Compound 72: N-(4-(3-Amino-1H-pyrazolo[3,4-b]pyridin-4-yl)-2-((1 S)-1-(4-fluorophenyl) ethoxy) phenyl)-1,1-difluoromethanesulfonamide

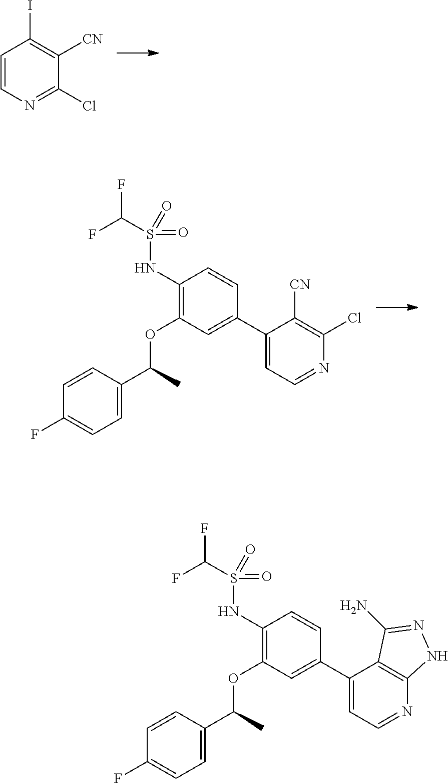 [SMILES: N#Cc1c(I)ccnc1Cl ; C[C@H](Oc1cc(-c2ccnc(Cl)c2C#N)ccc1NS(=O)(=O)C(F)F)c1ccc(F)cc1 ; C[C@H](Oc1cc(-c2ccnc3[nH]nc(N)c23)ccc1NS(=O)(=O)C(F)F)c1ccc(F)cc1]

### Step 1: N-(4-(2-Chloro-3-cyanopyridin-4-yl)-2-((1S)-1-(4-fluorophenyl)ethoxy)phenyl)-1,1-difluoromethanesulfonamide

To a mixture of 2-chloro-4-iodopyridine-3-carbonitrile (150 mg, 0.57 mmol), 1,1-difluoro-N-(2-((1 S)-1-(4-fluorophenyl)ethoxy)-4-(4,4,5,5-tetramethyl-1,3,2-dioxaborolan-2-yl)phenyl) methanesulfonamide (294 mg, 0.62 mmol) and Na₂CO₃ (119 mg, 1.13 mmol) in 1,4-dioxane/water (9/1, 6 mL) was added Pd(dppf)Cl₂—CH₂Cl₂ (46.3 mg, 0.06 mmol), and the reaction was heated at 100° C. under N₂ for 23 h. Once cooled, the mixture was concentrated in vacuo and the residue was partitioned between EtOAc (20 mL) and water (20 mL). The phases were separated, and the aqueous phase was extracted with EtOAc (3×20 mL). The combined organic fractions were washed with brine, dried over MgSO₄ and concentrated in vacuo. The residue was purified by column chromatography (0-50% EtOAc:n-Hep) to give the title product (214 mg, 78%) as a yellow solid. LCMS (Method D): 2.46 min, m/z 482.0 [M+H]⁺. ¹H NMR (300 MHz, DMSO-d₆): 10.61 (s, 1H), 8.70 (d, J=4.1 Hz, 1H), 7.61-7.57 (m, 3H), 7.43 (d, J=8.2 Hz, 1H), 7.31 (d, J=1.8 Hz, 1H), 7.21-7.15 (m, 3H), 7.04 (t, J=52.3 Hz, 1H), 5.70 (q, J=6.2 Hz, 1H), 1.59 (d, J=6.2 Hz, 3H).

### Step 2: N-(4-(3-Amino-1H-pyrazolo[3,4-b]pyridin-4-yl)-2-((1 S)-1-(4-fluorophenyl) ethoxy)phenyl)-1,1-difluoromethanesulfonamide

To a solution of N-(4-(2-chloro-3-cyanopyridin-4-yl)-2-((1 S)-1-(4-fluorophenyl) ethoxy)phenyl)-1,1-difluoromethanesulfonamide (210 mg, 0.44 mmol) in i-PrOH (5 mL) was added NH₂NH₂—H₂O (63.1 μL, 1.30 mmol), and the reaction was heated at 80° C. with μW irradiation for 1 h. Once cooled, the mixture was diluted with water (25 mL) and extracted with EtOAc (3×25 mL). The combined organics were washed with brine, dried over MgSO₄ and concentrated in vacuo. The residue was purified by column chromatography (0-10% MeOH:DCM) to give the title product (145 mg, 70%) as a yellow solid. LCMS (Method D): 1.86 min, m/z 478.0 [M+H]⁺. ¹H NMR (300 MHz, DMSO-d₆): 10.51 (s, 1H), 8.35 (d, J=4.7 Hz, 1H), 7.58 (dd, J=8.7, 5.5 Hz, 2H), 7.40 (d, J=8.0 Hz, 1H), 7.18 (d, J=8.9 Hz, 2H), 7.15-7.07 (m, 2H), 7.03 (t, J=51.6 Hz, 1H), 6.77 (d, J=4.7 Hz, 1H), 5.72 (q, J=6.3 Hz, 1H), 4.47 (s, 2H), 1.59 (d, J=6.3 Hz, 3H).

### Compound 65: N-(4-(3-amino-1H-pyrazolo[4,3-c]pyridin-4-yl)-2-((4-fluorophenyl)methoxy) phenyl)-2,2,2-trifluoroethane-1-sulfonamide

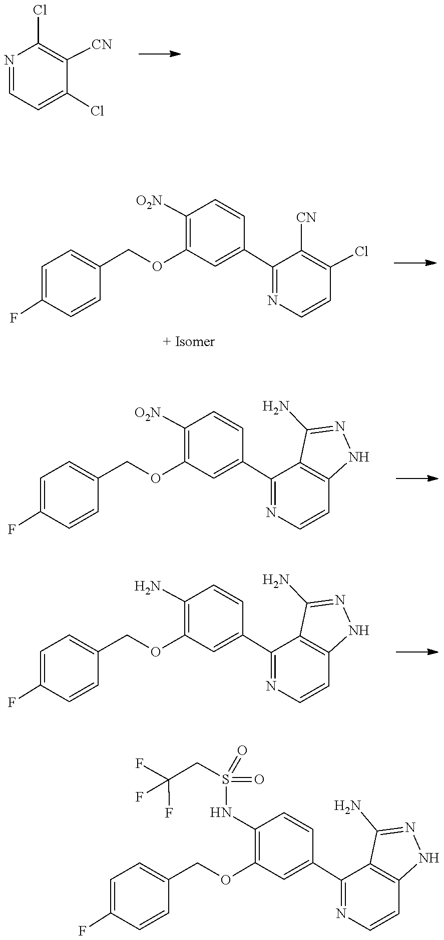 [SMILES: N#Cc1c(Cl)ccnc1Cl ; N#Cc1c(Cl)ccnc1-c1ccc([N+](=O)[O-])c(OCc2ccc(F)cc2)c1 ; Nc1n[nH]c2ccnc(-c3ccc([N+](=O)[O-])c(OCc4ccc(F)cc4)c3)c12 ; Nc1ccc(-c2nccc3[nH]nc(N)c23)cc1OCc1ccc(F)cc1 ; Nc1n[nH]c2ccnc(-c3ccc(NS(=O)(=O)CC(F)(F)F)c(OCc4ccc(F)cc4)c3)c12]

### Step 1: 4-Chloro-2-(3-((4-fluorophenyl)methoxy)-4-nitrophenyl)pyridine-3-carbonitrile

To a mixture of 2,4-dichloropyridine-3-carbonitrile (692 mg, 4.0 mmol), 2-(3-((4-fluorophenyl) methoxy)-4-nitrophenyl)-4,4,5,5-tetramethyl-1,3,2-dioxaborolane (1.64 g, 4.40 mmol) and Na₂CO₃ (847 mg, 8.00 mmol) in 1,4-dioxane:water (9:1, 40 mL) was added Pd(dppf)Cl₂ (292 mg, 0.4 mmol), and the reaction was heated at 100° C. under N₂ for 18 h. Once cooled, the mixture was diluted with EtOAc, filtered over Celite and concentrated in vacuo. The residue was purified by column chromatography (0-50% EtOAc:c-Hex) to give the title compound & it's regioisomer (5:2, 1.02 g, 48%) as a white solid. LCMS (Method D): 2.47 min, m/z 384.0 [M+H]⁺.

### Step 2: 4-(3-((4-Fluorophenyl)methoxy)-4-nitrophenyl)-1H-pyrazolo[4,3-c]pyridin-3-amine

To a mixture of 4-chloro-2-(3-((4-fluorophenyl)methoxy)-4-nitrophenyl)pyridine-3-carbonitrile & it's regioisomer (191 mg, 0.5 mmol) in i-PrOH (5 mL) was added NH₂NH₂—H₂O (48.4 μL, 1.0 mmol), and the reaction was heated at 80° C. with μW irradiation for 1 h. Once cooled, the precipitate was collected via filtration to give the title product (133 mg, 70%) as a yellow solid. LCMS (Method D): 1.46 min, m/z 380.9 [M+H]⁺. ¹H NMR (300 MHz, DMSO-d₆): 12.34 (s, 1H), 8.29 (d, J=5.9 Hz, 1H), 8.06 (d, J=8.3 Hz, 1H), 7.77 (d, J=1.6 Hz, 1H), 7.55-7.47 (m, 3H), 7.33 (d, J=5.9 Hz, 1H), 7.26 (dd, J=8.9 Hz, 2H), 5.38 (s, 2H), 4.96 (s, 2H).

### Step 3: 4-(4-Amino-3-((4-fluorophenyl)methoxy)phenyl)-1H-pyrazolo[4,3-c]pyridin-3-amine

To a solution of 4-(3-((4-fluorophenyl)methoxy)-4-nitrophenyl)-1H-pyrazolo[4,3-c]pyridin-3-amine (210 mg, 0.55 mmol) in EtOH:THF:H₂O (4:4:1, 18 mL) were added Fe (154 mg, 2.76 mmol) and NH₄Cl (29.6 mg, 0.55 mmol), and the reaction was heated at reflux overnight. Whilst hot, the mixture was filtered over Celite and the pad was rinsed with EtOH. The combined filtrates were concentrated in vacuo and the residue was purified by column chromatography (0-20% MeOH:DCM) to give the title product (127 mg, 66%) as a yellow glass. LCMS (Method D): 1.21 min, m/z 350.0 [M+H]⁺. ¹H NMR (300 MHz, DMSO-d₆): 12.33 (bs, 1H), 8.13 (d, J=6.2 Hz, 1H), 7.57 (dd, J=8.9, 5.6 Hz, 2H), 7.32 (d, J=1.8 Hz, 1H), 7.25-7.19 (m, 3H), 7.12 (dd, J=8.0, 1.8 Hz, 1H), 6.82 (d, J=8.0 Hz, 1H), 5.28 (s, 2H), 5.16 (s, 2H), 4.94 (s, 2H).

### Step 4: N-(4-(3-amino-1H-pyrazolo[4,3-c]pyridin-4-yl)-2-((4-fluorophenyl)methoxy) phenyl)-2,2,2-trifluoroethane-1-sulfonamide

To a solution of 4-(4-amino-3-((4-fluorophenyl)methoxy)phenyl)-1H-pyrazolo [4,3-c]pyridin-3-amine (120 mg, 0.34 mmol) in pyridine (3.43 mL) was added 2,2,2-trifluoroethanesulfonyl chloride (125 mg, 0.69 mmol), and the reaction was stirred at RT under N₂ overnight. The mixture was concentrated in vacuo and the residue was azeotropically distilled with MePh. The residue was purified by column chromatography (0-20% MeOH:DCM) followed by prep-HPLC purification (method B, gradient 30-60% MeCN) to give the title product (13.4 mg, 8%) as a yellow solid (as a formate salt). LCMS (Method D): 1.50 min, m/z 496.0 [M+H]⁺. ¹H NMR (300 MHz, DMSO-d₆): 12.35 (s, 2H), 9.90 (s, 1H), 8.24 (d, J=6.0 Hz, 1H), 8.13 (s, 1H), 7.66-7.56 (m, 2H), 7.51 (d, J=1.8 Hz, 1H), 7.44 (d, J=8.1 Hz, 1H), 7.31 (dd, J=8.1, 1.8 Hz, 1H), 7.29-7.18 (m, 3H), 5.23 (s, 2H), 4.88 (s, 2H), 4.35 (q, J=9.9 Hz, 2H).

### Compound 68: N-(4-(3-amino-1H-pyrazolo[4,3-c]pyridin-4-yl)-2-((4-fluorophenyl)methoxy) phenyl)-1,1-difluoromethanesulfonamide

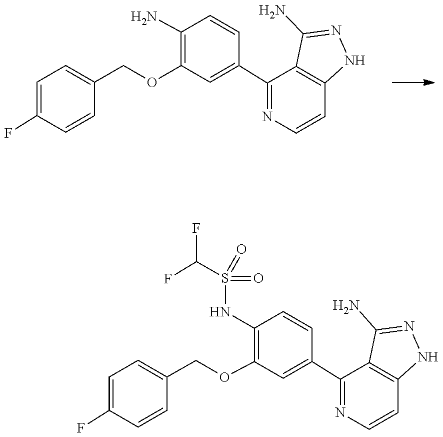 [SMILES: Nc1ccc(-c2nccc3[nH]nc(N)c23)cc1OCc1ccc(F)cc1 ; Nc1n[nH]c2ccnc(-c3ccc(NS(=O)(=O)C(F)F)c(OCc4ccc(F)cc4)c3)c12]

### Step 1: N-(4-(3-amino-1H-pyrazolo[4,3-c]pyridin-4-yl)-2-((4-fluorophenyl)methoxy) phenyl)-1,1-difluoromethanesulfonamide

To a solution of 4-(4-amino-3-((4-fluorophenyl)methoxy)phenyl)-1H-pyrazolo [4,3-c]pyridin-3-amine (160.3 mg, 0.46 mmol) in CHCl₃ (4.57 mL) were added pyridine (109 μL, 1.37 mmol) and difluoromethanesulfonyl chloride (60.7 μL, 0.69 mmol), and the reaction was stirred at RT under N₂ overnight. The mixture was concentrated in vacuo and the residue was purified by column chromatography (0-10% MeOH:DCM) followed by prep-HPLC purification (method B, gradient 30-60% MeCN) to give the title product (5.34 g, 3%) as a yellow solid. LCMS (Method D): 1.98 min, m/z 464.0 [M+H]⁺. ¹H NMR (300 MHz, DMSO-d₆): 8.62 (d, J=5.8 Hz, 1H), 7.64 (d, J=5.8 Hz, 1H), 7.58 (dd, J=8.7, 5.6 Hz, 2H), 7.28 (d, J=1.9 Hz, 1H), 7.26-7.16 (m, 2H), 7.07 (dd, J=8.0, 1.8 Hz, 1H), 6.82 (d, J=8.1 Hz, 1H), 5.89 (s, 2H), 5.30 (s, 2H), 5.14 (s, 2H).

### Compound 69: N-(4-(3-Amino-1H-pyrazolo[3,4-d]pyrimidin-4-yl)-2-((4-fluorophenyl)methoxy) phenyl)-1,1-difluoromethanesulfonamide

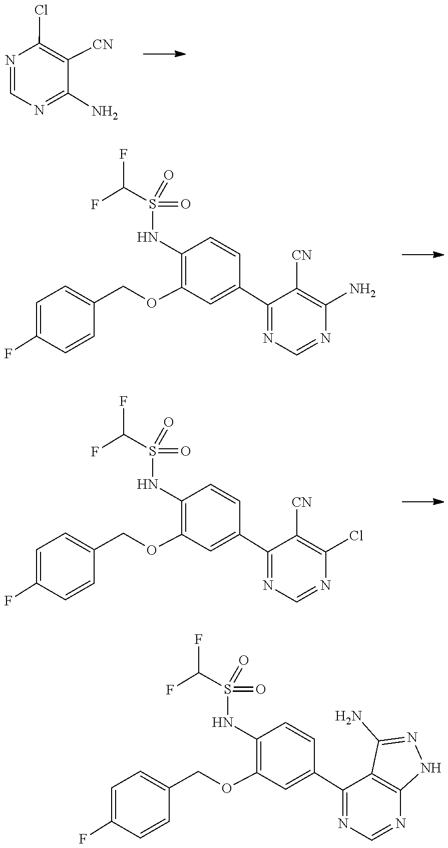 [SMILES: N#Cc1c(N)ncnc1Cl ; N#Cc1c(N)ncnc1-c1ccc(NS(=O)(=O)C(F)F)c(OCc2ccc(F)cc2)c1 ; N#Cc1c(Cl)ncnc1-c1ccc(NS(=O)(=O)C(F)F)c(OCc2ccc(F)cc2)c1 ; Nc1n[nH]c2ncnc(-c3ccc(NS(=O)(=O)C(F)F)c(OCc4ccc(F)cc4)c3)c12]

### Step 1: N-(4-(6-Amino-5-cyanopyrimidin-4-yl)-2-((4-fluorophenyl) methoxy)phenyl)-1,1-difluoromethanesulfonamide

To a mixture of 4-amino-6-chloropyrimidine-5-carbonitrile (76.0 mg, 0.49 mmol), 1,1-difluoro-N-(2-((4-fluorophenyl)methoxy)-4-(4,4,5,5-tetramethyl-1,3,2-dioxaborolan-2-yl)phenyl) methanesulfonamide (225 mg, 0.49 mmol) and Na₂CO₃ (104 mg, 0.98 mmol) in dioxane:H₂O (9:1, 5.0 mL) was added and Pd(dppf)Cl₂—CH₂Cl₂ (40.1 mg, 0.05 mmol), and the reaction was heated at 100° C. under N₂ for 24 h. Once cooled, the mixture was diluted with water (20 mL) and extracted with EtOAc (3×20 mL). The combined organics were washed with brine, dried over MgSO₄ and concentrated in vacuo. The residue was purified by column chromatography (0-100% EtOAc:n-Hep) to give the title product (93.1 mg, 42%) as a yellow solid. LCMS (Method D): 1.92 min, m/z 450.0 [M+H]⁺. ¹H NMR (300 MHz, DMSO-d₆): 10.61 (s, 1H), 8.60 (d, J=2.8 Hz, 1H), 7.94 (bs, 2H), 7.63-7.58 (m, 3H), 7.49 (dd, J=8.2, 1.9 Hz, 1H), 7.42 (d, J=8.2 Hz, 1H), 7.27-7.21 (m, 2H), 6.96 (t, J=52.4 Hz, 1H), 5.21 (s, 2H).

### Step 2: N-(4-(6-Chloro-5-cyanopyrimidin-4-yl)-2-((4-fluorophenyl)methoxy)phenyl)-1,1-difluoromethanesulfonamide

To a solution of N-(4-(6-amino-5-cyanopyrimidin-4-yl)-2-((4-fluorophenyl)methoxy) phenyl)-1,1-difluoromethanesulfonamide (93.1 mg, 0.21 mmol) in MeCN (2.0 mL) were added CuCl₂ (55.6 mg, 0.41 mmol) and Isoamyl nitrite (55.6 μL, 0.41 mmol), and the reaction was heated at 65° C. for 5 h. Once cooled, the mixture was acidified to pH 3 with 1 M HCl and extracted with DCM (10 mL). The organic phase was dried over MgSO₄ and concentrated in vacuo. The residue was purified by column chromatography (0-50% EtOAc:n-Hep) to give the title product (26.0 mg, 27%) as a yellow solid. LCMS (Method D): 2.45 min, m/z 469.0 [M+H]⁺. ¹H NMR (300 MHz, CDCl₃): 9.12 (s, 1H), 7.87-7.75 (m, 3H), 7.44-7.40 (m, 2H), 7.30 (s, 1H), 7.13 (dd, J=8.7 Hz, 2H), 6.31 (t, J=53.5 Hz, 1H), 5.21 (s, 2H).

### Step 3: N-(4-(3-Amino-1H-pyrazolo[3,4-d]pyrimidin-4-yl)-2-((4-fluorophenyl)methoxy) phenyl)-1,1-difluoromethanesulfonamide

To a solution of N-(4-(6-chloro-5-cyanopyrimidin-4-yl)-2-((4-fluorophenyl)methoxy) phenyl)-1,1-difluoromethanesulfonamide (26 mg, 0.06 mmol) in i-PrOH (554 μL) was added NH₂NH₂—H₂O (5.37 μL, 0.11 mmol) and the reaction was heated at 80° C. in a sealed tube for 1 h. Once cooled, the precipitate was collected via filtration to give the title product (4.20 mg, 16%) as a yellow solid. LCMS (Method D): 1.68 min, m/z 465.0 [M+H]⁺. ¹H NMR (300 MHz, DMSO-d₆): 9.29 (s, 1H), 8.59 (s, 1H), 7.58 (ddd, J=8.6, 5.5, 2.6 Hz, 2H), 7.52 (d, J=2.0 Hz, 1H), 7.44 (dd, J=8.3, 2.0 Hz, 1H), 7.38 (d, J=8.3 Hz, 1H), 7.26-7.16 (m, 2H), 6.61 (t, J=53.8 Hz, 1H), 5.12 (s, 2H).

### Compound 70: N-(4-(3-Amino-6-methyl-1H-pyrazolo[3,4-b]pyridin-4-yl)-2-((4-fluorophenyl) methoxy)phenyl)-1,1-difluoromethanesulfonamide

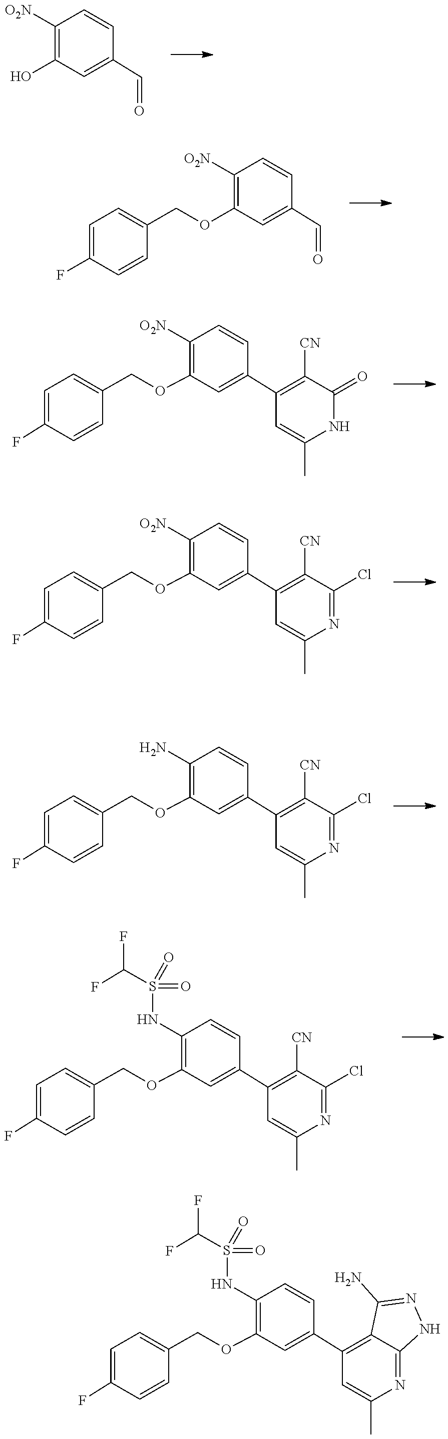

### Step 1: 3-((4-Fluorophenyl)methoxy)-4-nitrobenzaldehyde

To a solution of 3-hydroxy-4-nitrobenzaldehyde (2.0 g, 11.9 mmol) in MeCN (24 mL) were added K₂CO₃ (1.96 g, 14.2 mmol) and 4-fluorobenzyl bromide (1.54 mL, 12.4 mmol), and the reaction was heated at reflux for 2 h. Once cooled, water (100 mL) was added and the mixture was extracted with Et₂O (4×50 mL). The combined organics were washed with brine, dried over MgSO₄ and concentrated in vacuo to give the title product (7.33 g, 99%) as a pale yellow solid. LCMS (Method D): 2.24 min, m/z 276.0 [M+H]⁺. ¹H NMR (300 MHz, CDCl₃): 10.03 (s, 1H), 7.94 (d, J=8.1 Hz, 1H), 7.63 (d, J=1.5 Hz, 1H), 7.56 (dd, 8.1, 1.5 Hz, 1H), 7.46-7.41 (m, 2H), 7.12-7.06 (m, 2H), 5.26 (s, 2H).

### Step 2: 4-(3-((4-Fluorophenyl)methoxy)-4-nitrophenyl)-6-methyl-2-oxo-1,2-dihydropyridine-3-carbonitrile

To a suspension of AcONH₄ (2.23 g, 29.0 mmol) in n-BuOH (15 mL) were added acetone (267 μL, 3.63 mmol), methyl 2-cyanoacetate (319 μL, 3.63 mmol) and 3-((4-fluorophenyl)methoxy)-4-nitrobenzaldehyde (1.0 g, 3.63 mmol), and the reaction was heated at reflux for 3 h. Once cooled, the precipitate was collected via filtration, washed with EtOH and n-Hep, and air-dried to give the title product (351 mg, 26%) as a pale orange solid. LCMS (Method D): 1.99 min, m/z 378.8 [M−H]⁻. ¹H NMR (300 MHz, DMSO-d₆): 8.04 (d, J=8.4 Hz, 1H), 7.65 (d, J=1.6 Hz, 1H), 7.53-7.49 (m, 2H), 7.34 (dd, J=8.4, 1.6 Hz, 1H), 7.28-7.22 (m, 2H), 6.36 (s, 1H) 5.33 (s, 2H), 2.33 (s, 3H).

### Step 3: 2-Chloro-4-(3-((4-fluorophenyl)methoxy)-4-nitrophenyl)-6-methylpyridine-3-carbonitrile

A mixture of 4-(3-((4-fluorophenyl)methoxy)-4-nitrophenyl)-6-methyl-2-oxo-1,2-dihydropyridine-3-carbonitrile (0.43 g, 1.13 mmol, from multiple iterations) and PCl₅ (470 mg, 2.26 mmol) in POCl₃ (5 mL) was heated at reflux for 5 h. Once cooled, the mixture was poured into ice-water and neutralised with sat. NaHCO₃. The precipitate was collected via filtration, washed with water and dried under vacuum for 15 h to give the title product (425 mg, 95%) as a light orange solid. LCMS (Method D): 2.64 min, m/z 398.0 [M+H]⁺. ¹H NMR (300 MHz, DMSO-d₆): 8.09 (d, J=8.3 Hz, 1H), 7.81 (d, J=1.6 Hz, 1H), 7.70 (s, 1H), 7.54-7.49 (m, 2H), 7.45 (dd, J=8.3, 1.6 Hz, 1H), 7.28-7.23 (m, 2H), 5.34 (s, 2H), 2.63 (s, 3H).

### Step 4: 4-(4-Amino-3-((4-fluorophenyl)methoxy)phenyl)-2-chloro-6-methylpyridine-3-carbonitrile

To a solution of 2-chloro-4-(3-((4-fluorophenyl)methoxy)-4-nitrophenyl)-6-methylpyridine-3-carbonitrile (425 mg, 1.06 mmol) in EtOH (10.6 mL) was added SnCl₂·2H₂O (1.19 g, 5.30 mmol) in portions, and the reaction was heated at 70° C. under N₂ for 2 h. Once cooled, the mixture was poured into NH₄OH (25%, 25 mL) and extracted with EtOAc (3×25 mL). The combined organics were washed with brine (2×25 mL), dried over MgSO₄ and concentrated in vacuo. The residue was purified by column chromatography (0-10% MeOH:DCM) to give the title product (94.7 mg, 24%) as a brown solid. LCMS (Method D): 2.45 min, m/z 368.0 [M+H]⁺. ¹H NMR (300 MHz, DMSO-d₆): 7.59-7.54 (m, 3H), 7.30 (d, J=2.0 Hz, 1H), 7.24-7.14 (m, 3H), 6.78 (d, J=8.2 Hz, 1H), 5.48 (s, 2H), 5.15 (s, 2H), 2.54 (s, 3H).

### Step 5: N-(4-(2-Chloro-3-cyano-6-methylpyridin-4-yl)-2-((4-fluorophenyl)methoxy) phenyl)-1,1-difluoromethanesulfonamide

To a solution of 4-(4-amino-3-((4-fluorophenyl)methoxy)phenyl)-2-chloro-6-methyl pyridine-3-carbonitrile (90 mg, 0.24 mmol) in CHCl₃ (2.5 mL) were added pyridine (98.2 μL, 1.22 mmol) and difluoromethanesulfonyl chloride (43.3 μL, 0.49 mmol), and the reaction was stirred at RT under N₂ overnight. The mixture was concentrated in vacuo and the residue was azeotropically distilled with MePh. The residue was purified by column chromatography (0-50% EtOAc:n-Hep) to give the title product (65.9 mg, 56%) as a yellow solid. LCMS (Method D): 2.80 min, m/z 480.8 [M−H]⁻. ¹H NMR (300 MHz, DMSO-d₆): 7.72-7.64 (m, 3H), 7.57-7.52 (m, 3H), 7.49 (t, J=51.5 Hz, 1H), 7.44 (dd, J=8.2, 1.9 Hz, 1H), 7.26 (dd, J=8.9 Hz, 2H), 5.29 (s, 2H), 2.62 (s, 3H).

### Step 6: N-(4-(3-Amino-6-methyl-1H-pyrazolo[3,4-b]pyridin-4-yl)-2-((4-fluorophenyl)methoxy) phenyl)-1,1-difluoromethanesulfonamide

To a solution of N-(4-(2-chloro-3-cyano-6-methylpyridin-4-yl)-2-((4-fluorophenyl)methoxy) phenyl)-1,1-difluoromethanesulfonamide (65 mg, 0.13 mmol) in i-PrOH (2 mL) was added NH₂NH₂—H₂O (12.9 μL, 0.27 mmol) and the reaction was heated at 80° C. with μW irradiation for 1 h. Once cooled, the mixture was partitioned between EtOAc (10 mL) and water (10 mL). The phases were separated, and the aqueous fraction was extracted with EtOAc (2×10 mL). The combined organics were washed with brine (10 mL), dried over MgSO₄ and concentrated in vacuo. The residue was purified by column chromatography (0-10% MeOH:DCM) to give the title product (16.3 mg, 25%) as a yellow solid. LCMS (Method D): 1.83 min. m/z 478.0 [M+H]⁺. ¹H NMR (300 MHz, DMSO-d₆): 12.14 (s, 1H), 10.48 (s, 1H), 7.61 (dd, J=8.5, 5.7 Hz, 2H), 7.42 (d, J=8.0 Hz, 1H), 7.38 (d, J=1.8 Hz, 1H), 7.25 (t, J=8.9 Hz, 2H), 7.18 (dd, J=8.1, 1.8 Hz, 1H), 6.94 (t, J=52.7 Hz, 1H), 6.85 (s, 1H), 5.25 (s, 2H), 4.55 (s, 2H), 2.54 (s, 3H).

### Compound 71: N-(4-(3-Amino-6-phenyl-1H-pyrazolo[3,4-b]pyridin-4-yl)-2-((4-fluorophenyl) methoxy)phenyl)-1,1-difluoromethanesulfonamide

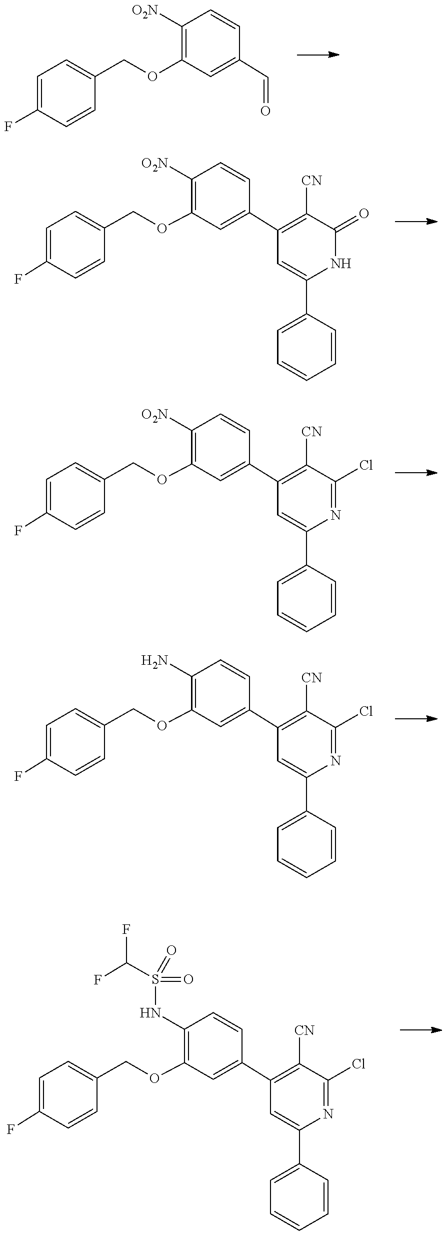

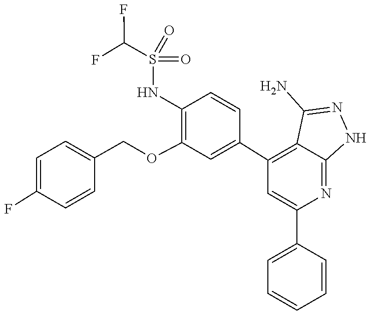 [SMILES: Nc1n[nH]c2nc(-c3ccccc3)cc(-c3ccc(NS(=O)(=O)C(F)F)c(OCc4ccc(F)cc4)c3)c12]

### Step 1: 4-(3-((4-Fluorophenyl)methoxy)-4-nitrophenyl)-2-oxo-6-phenyl-1,2-dihydropyridine-3-carbonitrile

To a suspension of AcONH₄ (2.23 g, 29.0 mmol) in n-BuOH (15 mL) were successively added acetophenone (423 μL, 3.63 mmol), methyl 2-cyanoacetate (319 μL, 3.63 mmol) and 3-((4-fluorophenyl)methoxy)-4-nitrobenzaldehyde (1.0 g, 3.63 mmol), and the reaction was heated at reflux for 3 h. Once cooled, the precipitate was collected via filtration, washed with EtOH and n-Hep, and air-dried to give the title product (687 mg, 43%) as a pale orange solid. LCMS (Method D): 2.38 min, m/z 442.0 [M+H]⁺. ¹H NMR (300 MHz, DMSO-d₆): 8.07 (d, J=8.3 Hz, 1H), 7.90 (d, J=7.9 Hz, 2H), 7.78 (d, J=1.7 Hz, 1H), 7.59-7.46 (m, 6H), 7.29-7.21 (m, 2H), 6.90 (d, J=1.7 Hz, 1H) 5.37 (s, 2H).

### Step 2: 2-Chloro-4-(3-((4-fluorophenyl)methoxy)-4-nitrophenyl)-6-phenylpyridine-3-carbonitrile

A mixture of 4-(3-((4-fluorophenyl)methoxy)-4-nitrophenyl)-2-oxo-6-phenyl-1,2-dihydropyridine-3-carbonitrile (680 mg, 1.54 mmol) and PCl₅ (641 mg, 3.08 mmol) in POCl₃ (7 mL) was heated at reflux for 5 h. Once cooled, the mixture was poured into ice-water and neutralised with sat. NaHCO₃. The precipitate was collected via filtration, washed with water and dried under vacuum for 15 h to give the title product (703 mg, 99%) as a pale yellow solid. LCMS (Method D): 2.37 min, molecular ion not observed. ¹H NMR (300 MHz, DMSO-d₆): 8.08 (d, J=8.3 Hz, 1H), 7.91 (d, J=7.0 Hz, 2H), 7.79 (d, J=1.6 Hz, 1H), 7.60-7.47 (m, 6H), 7.28-7.22 (m, 2H), 6.92 (s, 1H) 5.37 (s, 2H).

### Step 3: 4-(4-Amino-3-((4-fluorophenyl)methoxy)phenyl)-2-chloro-6-phenylpyridine-3-carbonitrile

To a solution of 2-chloro-4-(3-((4-fluorophenyl)methoxy)-4-nitrophenyl)-6-phenylpyridine-3-carbonitrile (700 mg, 1.52 mmol) in EtOH (15 mL) was added SnCl₂·2H₂O (1.73 g, 7.70 mmol) in portions, and the reaction was heated at 70° C. under N₂ for 2 h. Once cooled, the mixture was poured into NH₄OH (25%, 25 mL) and extracted with EtOAc (3×50 mL). The combined organics were washed with brine (2×50 mL), dried over MgSO₄ and concentrated in vacuo. The residue was purified by column chromatography (0-10% MeOH:DCM) to give the title product (48.5 mg, 7%) as a yellow glass. LCMS (Method D): 3.04 min, m/z 430.0 [M+H]⁺. ¹H NMR (300 MHz, DMSO-d₆): 8.21-8.18 (m, 2H), 8.08 (s, 1H), 7.61-7.54 (m, 5H), 7.41 (d, J=2.0 Hz, 1H), 7.29 (dd, J=8.2, 2.0 Hz, 1H), 7.25-7.19 (m, 2H), 6.82 (d, J=8.2 Hz, 1H) 5.52 (s, 2H), 5.19 (s, 2H).

### Step 4: N-(4-(2-Chloro-3-cyano-6-phenylpyridin-4-yl)-2-((4-fluorophenyl)methoxy) phenyl)-1,1-difluoromethanesulfonamide

To a solution of 4-(4-amino-3-((4-fluorophenyl)methoxy)phenyl)-2-chloro-6-phenylpyridine-3-carbonitrile (45 mg, 0.10 mmol) in CHCl₃ (1.0 mL) were added pyridine (42.0 μL, 0.52 mmol) and difluoromethanesulfonyl chloride (18.4 μL, 0.21 mmol), and the reaction was stirred at RT under N₂ overnight. The mixture was concentrated in vacuo and the residue was azeotropically distilled with MePh. The residue was purified by column chromatography (0-20% EtOAc:n-Hep) to give the title product (48.5 mg, 85%) as a yellow solid. LCMS (Method D): 3.21 min, m/z 544.8 [M+H]⁺. ¹H NMR (300 MHz, DMSO-d₆): 8.34 (s, 1H), 8.28-8.25 (m, 2H), 7.79 (d, J=1.8 Hz, 1H), 7.69 (dd, J=8.1, 5.3 Hz, 1H), 7.60-7.53 (m, 7H), 7.51 (t, J=51.5 Hz, 1H), 7.29-7.23 (m, 2H), 5.33 (s, 2H).

### Step 5: N-(4-(3-Amino-6-phenyl-1H-pyrazolo[3,4-b]pyridin-4-yl)-2-((4-fluorophenyl)methoxy) phenyl)-1,1-difluoromethanesulfonamide

To a solution of N-(4-(2-chloro-3-cyano-6-phenylpyridin-4-yl)-2-((4-fluorophenyl)methoxy) phenyl)-1,1-difluoromethanesulfonamide (48 mg, 0.09 mmol) in i-PrOH (1.0 mL) was added NH₂NH₂—H₂O (8.55 μL, 0.18 mmol) and the reaction was heated at 80° C. with μW irradiation for 1 h. Once cooled, the mixture was partitioned between EtOAc (10 mL) and water (10 mL). The phases were separated, and the aqueous fraction was extracted with EtOAc (2×10 mL). The combined organics were washed with brine (10 mL), dried over MgSO₄ and concentrated in vacuo. The residue was purified by column chromatography (0-10% MeOH:DCM) to give the title product (6.50 mg, 14%) as a yellow solid. LCMS (Method D): 2.35 min, m/z 540.0 [M+H]⁺. ¹H NMR (300 MHz, DMSO-d₆): 12.40 (s, 1H), 10.53 (s, 1H), 8.16 (dd, J=8.3, 1.7 Hz, 2H), 7.64-7.55 (m, 2H), 7.52-7.46 (m, 6H), 7.31-7.22 (m, 3H), 6.95 (t, J=52.7 Hz, 1H), 5.29 (s, 2H), 4.65 (s, 2H).

### Compound 173: 4-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)cyclohexane-1-carboxylic acid

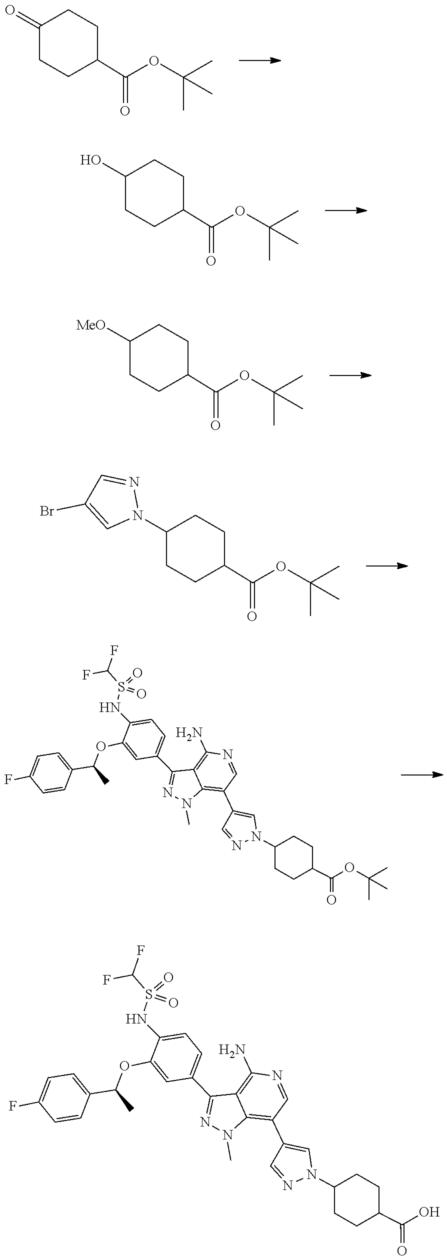 [SMILES: CC(C)(C)OC(=O)C1CCC(=O)CC1 ; CC(C)(C)OC(=O)C1CCC(O)CC1 ; COC1CCC(C(=O)OC(C)(C)C)CC1 ; CC(C)(C)OC(=O)C1CCC(n2cc(Br)cn2)CC1 ; C[C@H](Oc1cc(-c2nn(C)c3c(-c4cnn(C5CCC(C(=O)OC(C)(C)C)CC5)c4)cnc(N)c23)ccc1NS(=O)(=O)C(F)F)c1ccc(F)cc1 ; C[C@H](Oc1cc(-c2nn(C)c3c(-c4cnn(C5CCC(C(=O)O)CC5)c4)cnc(N)c23)ccc1NS(=O)(=O)C(F)F)c1ccc(F)cc1]

### Step 1: tert-butyl 4-hydroxycyclohexane-1-carboxylate

To a 0° C. solution of tert-butyl 4-oxocyclohexane-1-carboxylate (3 g, 15.1 mmol) in MeOH (20 mL) was added NaBH₄ (2.85 g, 75.5 mmol) in portions, and the mixture was stirred at 0° C. for 1 h. The mixture was concentrated under reduced pressure, then diluted with H₂O (60 mL) and extracted with Et₂O (3×80 mL). The combined organics were concentrated under reduced pressure to afford the title product (3.08 g, >100%) as yellow oil.

### Step 2: tert-butyl 4-(methanesulfonyloxy)cyclohexane-1-carboxylate

To a 0° C. mixture of tert-butyl 4-hydroxycyclohexane-1-carboxylate (4.36 g, 21.7 mmol) and Et₃N (10.9 g, 108 mmol) in MeCN (40 mL) was slowly added a solution of MsCl (4.97 g, 43.4 mmol) in MeCN (15 mL), and the reaction was stirred at 0° C. for 1 h. The mixture was concentrated under reduced pressure, then diluted with H₂O (120 mL) and extracted with EtOAc (3×150 mL). The combined organics were concentrated under reduced pressure to afford the title product (6.62 g, >100%) as yellow oil.

### Step 3: tert-butyl 4-(4-bromo-1H-pyrazol-1-yl)cyclohexane-1-carboxylate

A mixture of tert-butyl 4-(methanesulfonyloxy)cyclohexane-1-carboxylate (6.11 g, 21.9 mmol), Cs₂CO₃ (21.3 g, 65.6 mmol) and 4-bromo-1H-pyrazole (3.21 g, 21.9 mmol) in MeCN (60 mL) was stirred at 80° C. overnight. The mixture was concentrated under reduced pressure, then diluted with H₂O (100 mL) and extracted with EtOAc (3×120 mL). The combined organics were concentrated under reduced pressure and purified by column chromatography (PE:EA=30/1 to 20/1) to afford the title product (4.07 g, 56%) as a white solid. LCMS (Method B): 2.68 min, m/z 329.1, 331.1 [M+H]⁺.

### Step 4: tert-butyl 4-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)cyclohexane-1-carboxylate

A mixture of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (850 mg, 1.37 mmol), tert-butyl 4-(4-bromo-1H-pyrazol-1-yl)cyclohexane-1-carboxylate (583 mg, 1.78 mmol), B₂pin₂ (1.08 g, 4.26 mmol), Pd(OAc)₂ (260 mg, 1.16 mmol), butyldi-1-adamantylphosphine (695 mg, 1.94 mmol) and K₂CO₃ (803 mg, 5.81 mmol) in 1,4-Dioxane/H₂O (4/1, 30 mL) was stirred at 80° C. under N₂ for 2.5 h. The mixture was concentrated under reduced pressure, then diluted with H₂O (80 mL) and extracted with EtOAc (3×100 mL). The combined organics were concentrated under reduced pressure, and the residue was purified by column chromatography (DCM/MeOH=70/1 to 60:1) to afford the title product (488 mg, 24%) as a black solid. LCMS (Method B): 2.37 min, m/z 740.3 [M+H]⁺.

### Step 5: 4-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)cyclohexane-1-carboxylic acid

A solution of tert-butyl 4-(4-{4-amino-3-[4-(difluoromethanesulfonamido)-3-[(1S)-1-(4-fluoro phenyl)ethoxy]phenyl]-1-methyl-1H-pyrazolo[4,3-c]pyridin-7-yl}-1H-pyrazol-1-yl)cyclohexane-1-carboxylate (488 mg, 0.66 mmol) in HCOOH (15 mL) was stirred at RT under N₂ for 4 h. The mixture was concentrated under reduced pressure and the residue was purified by Prep-TLC (DCM/MeOH=10/1) to give the title product (80 mg, 18%) as a black solid. LCMS (Method B): 1.93 min; 682.2 [M−H]⁻. ¹H NMR (400 MHz, DMSO-d₆): 7.98 (s, 1H), 7.62-7.53 (m, 4H), 7.40 (d, J=8.0 Hz, 1H), 7.20-7.08 (m, 4H), 5.97 (s, 2H), 5.65 (q, J=6.3 Hz, 1H), 4.27 (dd, J=8.9, 4.4 Hz, 1H), 3.69 (s, 3H), 2.62-2.55 (m, 1H), 2.48 (s, 1H), 2.12 (s, 1H), 2.00 (dt, J=25.2, 8.5 Hz, 5H), 1.58 (d, J=6.2 Hz, 3H), 1.23 (s, 1H).

### Synthesis of VHL intermediates

### Intermediate 1: (2S,4R)-1-[(2S)-2-Amino-3,3-dimethylbutanoyl]-4-hydroxy-N-[[4-(4-methyl-1,3-thiazol-5-yl)phenyl]methyl]pyrrolidine-2-carboxamide hydrochloride

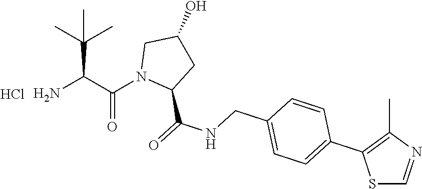 [SMILES: Cl ; Cc1ncsc1-c1ccc(CNC(=O)[C@@H]2C[C@@H](O)CN2C(=O)[C@@H](N)C(C)(C)C)cc1]

Intermediate 1 was prepared according to known methods exemplified in Carles Galdeano, Morgan S. Gadd, Pedro Soares, Salvatore Scaffidi, Inge Van Molle, Ipek Birced, Sarah Hewitt, David M. Dias, and Alessio Ciulli, Journal of Medicinal Chemistry, 2014, 57 (20), 8657-8663. LCMS (method E): 2.54 min, m/z: 431.2 [M+H]⁺.

### Intermediate 2: (2S,4S)-1-[(2S)-2-Amino-3,3-dimethylbutanoyl]-4-hydroxy-N-[[4-(4-methyl-1,3-thiazol-5-yl)phenyl]methyl]pyrrolidine-2-carboxamide hydrochloride

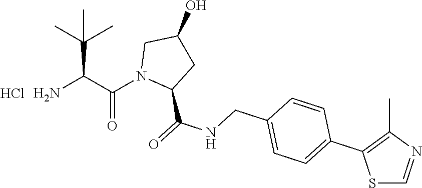 [SMILES: Cl ; Cc1ncsc1-c1ccc(CNC(=O)[C@@H]2C[C@H](O)CN2C(=O)[C@@H](N)C(C)(C)C)cc1]

Intermediate 2 was prepared according to the known procedure for Intermediate 1, using N-Boc-(2S,4S)-4-hydroxyproline in the appropriate step. LCMS (method G): 0.95 min, m/z: 431.0 [M+H]⁺.

### Intermediate 3: 2-(2-{[(2S,4R)-1-[(2S)-2-[(1-fluorocyclopropyl)formamido]-3,3-dimethyl butanoyl]-4-hydroxypyrrolidin-2-yl]formamido}-2-[4-(4-methyl-1,3-thiazol-5-yl)phenyl]ethoxy) acetic acid

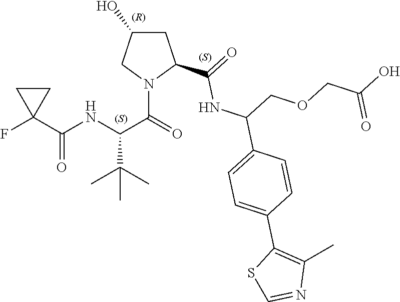

Intermediate 3 was prepared according to known methods exemplified in Kofink, C., Trainor, N., Mair, B. et al. Nat Commun, 13, 5969 (2022). LCMS (Method G): 1.49 min, m/z: 605.2 [M+H]⁺.

### Intermediate F1: tert-butyl 3-[4-(4,4,5,5-tetramethyl-1,3,2-dioxaborolan-2-yl)-1H-pyrazol-1-yl]propanoate

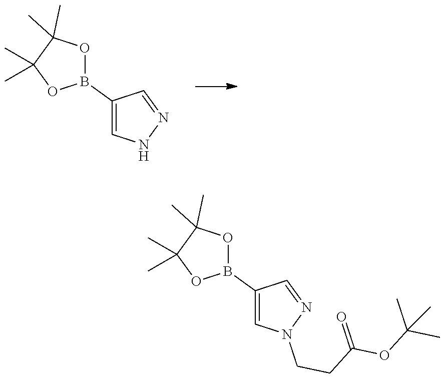 [SMILES: CC1(C)OB(c2cn[nH]c2)OC1(C)C ; CC(C)(C)OC(=O)CCn1cc(B2OC(C)(C)C(C)(C)O2)cn1]

### Step 1: tert-butyl 3-[4-(4,4,5,5-tetramethyl-1,3,2-dioxaborolan-2-yl)-1H-pyrazol-1-yl]propanoate

A mixture of 4-(4,4,5,5-tetramethyl-1,3,2-dioxaborolan-2-yl)-1H-pyrazole (200 mg, 1.03 mmol), tert-butyl 3-bromopropanoate (215 mg, 1.03 mmol), Cs₂CO₃ (1 g, 3.09 mmol) and NaI (77.1 mg, 515 μmol) in MeCN (40 mL) was stirred at 70° C. overnight. The mixture was concentrated and the residue was diluted with water (100 mL), then extracted with EtOAc (50 mL×2). The combined organics were dried (Na₂SO₄) and concentrated. The residue was purified by Prep-TLC (DCM/MeOH=20/1) to afford the title product (280 mg, 85%) as a brown solid. LCMS (method A): 3.99 min, m/z: 323.2 [M+H]⁺.

The following intermediates were similarly prepared from 4-(4,4,5,5-tetramethyl-1,3,2-dioxaborolan-2-yl)-1H-pyrazole and the appropriate ester according to the procedure for the synthesis of Intermediate F1.

TABLE 8
Intermediates F2-F5

| Inter- | | | | |
|---|---|---|---|---|
| mediate | Name | Structure | LCMS data | SM |
| F2 | tert-butyl 5-[4-(4,4,5,5- tetramethyl-1,3,2- dioxaborolan-2-yl)-1H- pyrazol-1-yl]pentanoate | 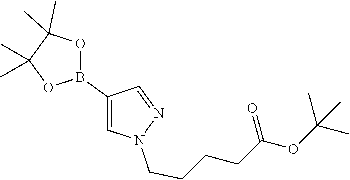 [SMILES: CC(C)(C)OC(=O)CCCCn1cc(B2OC(C)(C)C(C)(C)O2)cn1] | LCMS (method A): 4.20 min, m/z: 351.2 [M + H]⁺. | tert-butyl 5- bromopentanoate |
| F3 | N-(4-(4-amino-7-iodo- 1-methyl-1H- pyrazolo[4,3-c]pyridin- 3-yl)-2-((4- fluorobenzyl)oxy)phe- nyl)ethanesulfonamide | 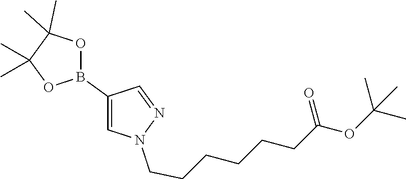 [SMILES: CC(C)(C)OC(=O)CCCCCCn1cc(B2OC(C)(C)C(C)(C)O2)cn1] | LCMS (method A): 3.69 min, m/z: 379.2 [M + H]⁺. | tert-butyl 7- bromoheptanoate |
| F4 | tert-butyl 6-[4-(4,4,5,5- tetramethyl-1,3,2- dioxaborolan-2-yl)-1H- pyrazol-1-yl]hexanoate | 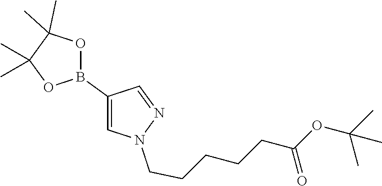 [SMILES: CC(C)(C)OC(=O)CCCCCn1cc(B2OC(C)(C)C(C)(C)O2)cn1] | LCMS (method A): 2.45 min, m/z: 365.2 [M + H]+. | tert-butyl 6- bromohexanoate |
| F5 | tert-butyl 8-[4-(4,4,5,5- tetramethyl-1,3,2- dioxaborolan-2-yl)-1H- pyrazol-1-yl]octanoate | 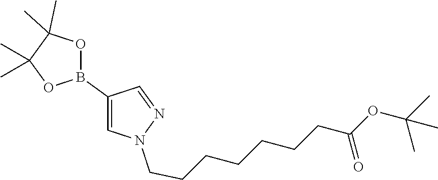 [SMILES: CC(C)(C)OC(=O)CCCCCCCn1cc(B2OC(C)(C)C(C)(C)O2)cn1] | LCMS (method A): 2.82 min, m/z: 393.2 [M + H]+. | tert-butyl 8- bromooctanoate |

### Intermediate F6: tert-butyl 2-{2-[4-(4,4,5,5-tetramethyl-1,3,2-dioxaborolan-2-yl)-1H-pyrazol-1-yl]ethoxy}acetate

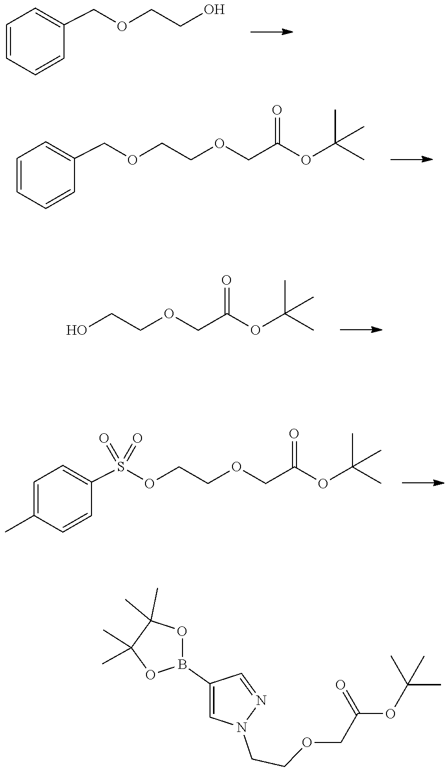

### Step 1: tert-butyl 2-[2-(benzyloxy)ethoxy]acetate

To a solution of 2-(benzyloxy)ethan-1-ol (1.0 g, 6.57 mmol) in DCM (13.1 mL) was added 37% aq. NaOH (13.1 mL), followed by tert-butyl 2-bromoacetate (3.86 mL, 26.2 mmol) and Bu₄NCl (1.82 g, 6.57 mmol), and the reaction was stirred at RT overnight. The mixture was extracted with EtOAc (3×25 mL), and the combined organics were washed with brine, dried (MgSO₄) and concentrated. The residue was purified by flash chromatography (0-20% EtOAc:heptane) to afford the title product (1.85 g, >100%) as a colourless oil. ¹H NMR (300 MHz, CDCl₃): 7.35-7.33 (m, 5H), 4.58 (s, 2H), 4.40 (s, 2H), 3.76-3.73 (m, 2H), 3.68-3.65 (m, 2H), 1.47 (s, 9H).

### Step 2: tert-butyl 2-(2-hydroxyethoxy)acetate

To a solution of tert-butyl 2-[2-(benzyloxy)ethoxy]acetate (1.86 g, 6.98 mmol) in EtOH (20 mL) was added 10% Pd/C (742 mg), and the reaction was stirred at RT under H₂ overnight. The reaction was filtered over Celite and concentrated to afford the title product (1.34 g, >100%) as a colourless oil. ¹H NMR (300 MHz, CDCl₃): 4.09 (s, 1H), 4.01 (s, 2H), 3.75-3.72 (m, 2H), 3.70-3.66 (m, 2H), 1.48 (s, 9H).

### Step 3: tert-butyl 2-{2-[(4-methylbenzenesulfonyl)oxy]ethoxy}acetate

To a solution of tert-butyl 2-(2-hydroxyethoxy)acetate (1.35 g, 7.66 mmol) in DCM (10 mL) were added p-TsCl (1.60 g, 8.42 mmol) and Et₃N (1.58 mL, 11.4 mmol), and the reaction was stirred at RT overnight. The mixture was concentrated and the residue was purified by flash chromatography (0-100% EtOAc:n-Hep) to afford the title product (1.66 g, 66%) as a colourless oil. ¹H NMR (300 MHz, CDCl₃): 7.80 (d, J=8.3 Hz, 2H), 7.34 (dd, J=0.7, 8.0 Hz, 2H), 4.21-4.17 (m, 2H), 3.94 (s, 2H), 3.78-3.75 (m, 2H), 2.44 (s, 3H), 1.46 (s, 9H).

### Step 4: tert-butyl 2-{2-[4-(4,4,5,5-tetramethyl-1,3,2-dioxaborolan-2-yl)-1H-pyrazol-1-yl]ethoxy}acetate

A mixture of tert-butyl 2-{2-[(4-methylbenzenesulfonyl)oxy]ethoxy}acetate (500 mg, 1.51 mmol), 4-(4,4,5,5-tetramethyl-1,3,2-dioxaborolan-2-yl)-1H-pyrazole (293 mg, 1.51 mmol), Cs₂CO₃ (1.47 g, 4.53 mmol) and NaI (113 mg, 755 μmol) in MeCN (40 mL) was stirred at 70° C. overnight. The mixture was diluted with H₂O (50 mL) and extracted with EtOAc (100 mL×3). The combined organics was dried (Na₂SO₄) and concentrated. The residue was purified by column chromatography (DCM/MeOH=20/1) to afford the title product (400 mg, 75%) as a yellow oil. LCMS: 2.64 min, m/z: 271.0 [M+H]⁺.

The following intermediates were similarly prepared from the appropriate alcohol according to the procedure for the synthesis of Intermediate F6.

TABLE 9
Intermediates F7 and F8

| Inter- | | | | |
|---|---|---|---|---|
| mediate | Name | Structure | LCMS data | SM |
| F7 | tert-butyl 2-(2-{2-[4- (4,4,5,5-tetramethyl-1,3,2- dioxaborolan-2-yl)-1H- pyrazol-1-yl]eth- oxy}ethoxy)acetate | 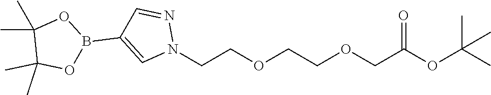 [SMILES: CC(C)(C)OC(=O)COCCOCCn1cc(B2OC(C)(C)C(C)(C)O2)cn1] | LCMS (method A): 3.94 min, m/z: 397.1 [M + H]⁺. | Diethylene Glycol Monobenzyl Ether |
| F8 | N-(4-(4-amino-7-iodo-1- methyl-1H-pyrazolo[4,3- c]pyridin-3-yl)-2-((4- fluorobenzyl)oxy)phe- nyl)ethanesulfoanmide | 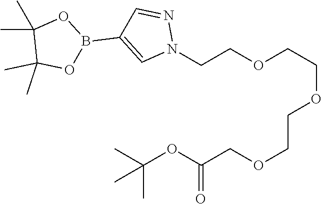 [SMILES: CC(C)(C)OC(=O)COCCOCCOCCn1cc(B2OC(C)(C)C(C)(C)O2)cn1] | LCMS (method A): 3.71 min, m/z: 441.3 [M + H]⁺. | Triethylene Glycol Monobenzyl Ether |

### Intermediate F9: tert-butyl 3-{3-[4-(4,4,5,5-tetramethyl-1,3,2-dioxaborolan-2-yl)-1H-pyrazol-1-yl]azetidin-1-yl}propanoate

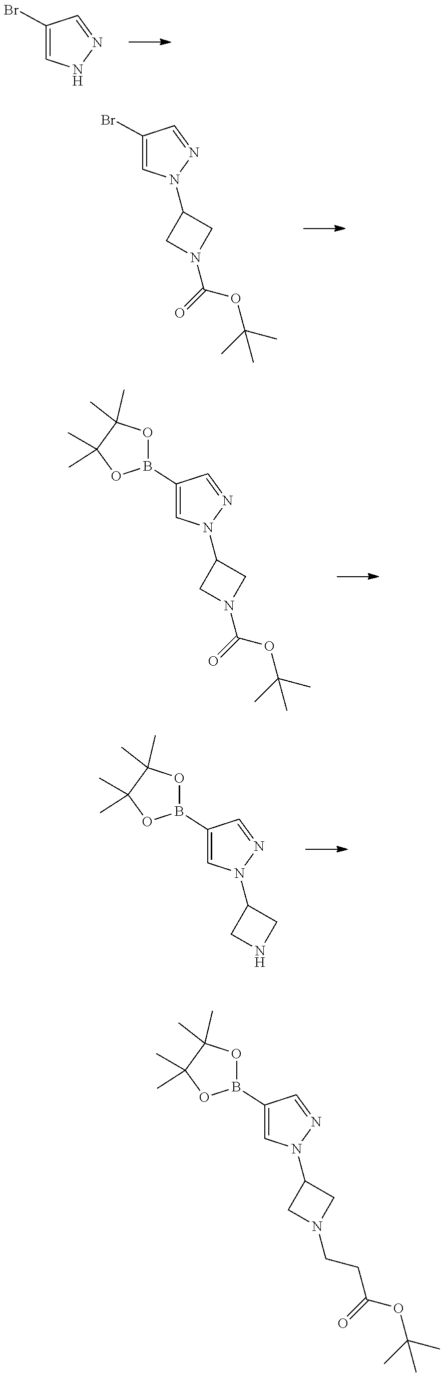 [SMILES: Brc1cn[nH]c1 ; CC(C)(C)OC(=O)N1CC(n2cc(Br)cn2)C1 ; CC(C)(C)OC(=O)N1CC(n2cc(B3OC(C)(C)C(C)(C)O3)cn2)C1 ; CC1(C)OB(c2cnn(C3CNC3)c2)OC1(C)C ; CC(C)(C)OC(=O)CCN1CC(n2cc(B3OC(C)(C)C(C)(C)O3)cn2)C1]

### Step 1: tert-butyl 3-(4-bromo-1H-pyrazol-1-yl)azetidine-1-carboxylate

To a mixture of tert-butyl 3-(methanesulfonyloxy)azetidine-1-carboxylate (7 g, 27.8 mmol) and 4-bromo-1H-pyrazole (4.48 g, 30.5 mmol) in DMF (200 ml) was added Cs₂CO₃ (27.1 g, 83.4 mmol), and the reaction was stirred at 80° C. for 3 h. Water was added and the mixture was extracted with EtOAc. The organic fraction was washed with brine, dried (Na₂SO₄) and concentrated. The residue was purified by column chromatography (PE/EtOAc=5/1, v/v) to afford the title compound (7.70 g, 91%) as a colourless oil. ¹H NMR (400 MHz, CDCl₃): 7.55 (s, 1H), 7.66 (s, 1H), 4.99 (p, 1H), 4.38-4.33 (m, 2H), 4.28-4.24 (m, 2H), 1.45 (s, 9H)

### Step 2: tert-butyl 3-[4-(4,4,5,5-tetramethyl-1,3,2-dioxaborolan-2-yl)-1H-pyrazol-1-yl]azetidine-1-carboxylate

To a mixture of tert-butyl 3-(4-bromo-1H-pyrazol-1-yl)azetidine-1-carboxylate (7.7 g, 25.4 mmol), AcOK (7.45 g, 76.1 mmol) and bis(pinacolato)diboron (9.64 g, 38.0 mmol) in 1,4-dioxane (500 mL) was added Pd(dppf)Cl₂ (1.85 g, 2.54 mmol), and the reaction was stirred at 100° C. under N₂ for 6 h. The mixture was concentrated and the residue was purified by column chromatography (PE/EtOAc=3/1, v/v) to afford the title compound (8.20 g, 92%) as a colorless oil. LCMS (method D): 1.39 min, m/z: 349.4 [M+H]⁺.

### Step 3: 1-(azetidin-3-yl)-4-(4,4,5,5-tetramethyl-1,3,2-dioxaborolan-2-yl)-1H-pyrazole

To a solution of tert-butyl 3-[4-(4,4,5,5-tetramethyl-1,3,2-dioxaborolan-2-yl)-1H-pyrazol-1-yl]azetidine-1-carboxylate (8.1 g, 23.1 mmol) in 1,4-dioxane (10 ml) was added 4 M HCl in dioxane (8.43 ml, 231 mmol), and the reaction was stirred at RT for 4 h. The mixture was concentrated to afford the title compound (6.20 g, >100%) as a light yellow oil. LCMS (method A): 1.30 min, m/z: 250.2 [M+H]⁺.

### Step 4: tert-butyl 3-{3-[4-(4,4,5,5-tetramethyl-1,3,2-dioxaborolan-2-yl)-1H-pyrazol-1-yl]azetidin-1-yl}propanoate

A mixture of 1-(azetidin-3-yl)-4-(4,4,5,5-tetramethyl-1,3,2-dioxaborolan-2-yl)-1H-pyrazole (500 mg, 2.00 mmol) and tert-butyl prop-2-enoate (512 mg, 4.00 mmol) in MeOH (5 mL) was stirred at 30° C. overnight. The mixture was concentrated to afford the title product (450 mg, 60%) as a yellow oil. LCMS (method A): 3.37 min, m/z: 378.3 [M+H]⁺.

### Intermediate F10: tert-butyl 2-{3-[4-(4,4,5,5-tetramethyl-1,3,2-dioxaborolan-2-yl)-1H-pyrazol-1-yl]pyrrolidin-1-yl}acetate

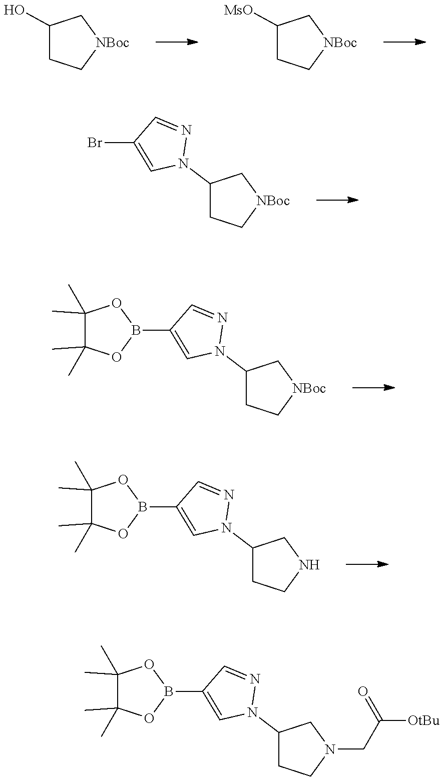 [SMILES: CC(C)(C)OC(=O)N1CCC(O)C1 ; CC(C)(C)OC(=O)N1CCC(OS(C)(=O)=O)C1 ; CC(C)(C)OC(=O)N1CCC(n2cc(Br)cn2)C1 ; CC(C)(C)OC(=O)N1CCC(n2cc(B3OC(C)(C)C(C)(C)O3)cn2)C1 ; CC1(C)OB(c2cnn(C3CCNC3)c2)OC1(C)C ; CC(C)(C)OC(=O)CN1CCC(n2cc(B3OC(C)(C)C(C)(C)O3)cn2)C1]

### Step 1: tert-butyl 3-(methanesulfonyloxy)pyrrolidine-1-carboxylate

To a 0° C. solution of tert-butyl 3-hydroxypyrrolidine-1-carboxylate (5 g, 26.7 mmol) in DCM (50 mL) were added Et₃N (5.40 g, 53.4 mmol) and MsCl (4.58 g, 40.0 mmol). The reaction was stirred at 0° C. for 1 h under N₂. The mixture was diluted with water and extracted with DCM (100 mL×2). The combined organics were washed with water (100 mL) and brine (30 mL), dried (Na₂SO₄) and concentrated to afford the title compound (7.90 g, >100%) as a white oil. ¹H NMR (400 MHz, DMSO-d₆): 5.24 (s, 1H), 3.48 (bs, 1H), 3.44-3.39 (m, 2H), 3.31-3.25 (m, 1H), 3.23 (s, 3H), 2.12 (bs, 2H), 1.40 (s, 9H).

### Step 2: tert-butyl 3-(4-bromo-1H-pyrazol-1-yl)pyrrolidine-1-carboxylate

To a solution of tert-butyl 3-(methanesulfonyloxy)pyrrolidine-1-carboxylate (7.9 g, 29.7 mmol) in DMF (90 mL) were added 4-bromo-1H-pyrazole (5.23 g, 35.6 mmol) and Cs₂CO₃ (29.0 g, 89.1 mmol), and the reaction was stirred at 100° C. under N₂ overnight. The mixture was diluted with water and extracted with EtOAc (100 ml×2). The combined organics were washed with water (100 mL) and brine (30 mL), dried (Na₂SO₄) and concentrated. The residue was purified by column chromatography (PE/EtOAc=10:1) to afford (7.70 g, 82%) as a colourless oil. ¹H NMR (400 MHz, DMSO-d₆): 8.06 (s, 1H), 7.58 (s, 1H), 4.93 (bs, 1H), 3.69-3.66 (m, 1H), 3.55-3.51 (m, 1H), 3.46-3.33 (m, 2H), 2.32-2.22 (m, 1H), 1.39 (s, 9H).

### Step 3: tert-butyl 3-[4-(4,4,5,5-tetramethyl-1,3,2-dioxaborolan-2-yl)-1H-pyrazol-1-yl]pyrrolidine-1-carboxylate

To a solution of tert-butyl 3-(4-bromo-1H-pyrazol-1-yl)pyrrolidine-1-carboxylate (4 g, 12.6 mmol) in 1,4-dioxane (60 mL) were added AcOK (3.70 g, 37.8 mmol), bis(pinacolato)diboron (3.83 g, 15.1 mmol) and Pd(dppf)Cl₂ (921 mg, 1.26 mmol), and the reaction was stirred at 100° C. under N₂ overnight. The mixture was concentrated and the residue was purified by column chromatography (PE/EtOAc=1/1, v/v) to afford the title compound (3.80 g, 83%) as a yellow oil. LCMS (method D): 1.59 min, m/z: 364.0 [M+H]⁺.

### Step 4: 1-(pyrrolidin-3-yl)-4-(4,4,5,5-tetramethyl-1,3,2-dioxaborolan-2-yl)-1H-pyrazole

To a solution of tert-butyl 3-[4-(4,4,5,5-tetramethyl-1,3,2-dioxaborolan-2-yl)-1H-pyrazol-1-yl]pyrrolidine-1-carboxylate (1 g, 2.75 mmol) in 1,4-dioxane (11 mL) was added 4M HCl in dioxane (11 mL), and the reaction was stirred at RT for 1 h under N₂. The mixture was concentrated to afford the title product (824 mg, >100%) as a white oil. LCMS (method B): 0.43 min, m/z: 264.0 [M+H]⁺.

### Step 5: tert-butyl 2-{3-[4-(4,4,5,5-tetramethyl-1,3,2-dioxaborolan-2-yl)-1H-pyrazol-1-yl]pyrrolidin-1-yl}acetate

To a solution of 1-(pyrrolidin-3-yl)-4-(4,4,5,5-tetramethyl-1,3,2-dioxaborolan-2-yl)-1H-pyrazole (145 mg, 551 μmol) in MeCN (5 mL) were added K₂CO₃ (379 mg, 2.75 mmol) and tert-butyl 2-bromoacetate (139 mg, 716 μmol), and the reaction was stirred at 25° C. for 1 h. The mixture was concentrated, and the residue was purified by column chromatography (DCM/MeOH=20/1, v/v) to afford the title compound (110 mg, 53%) as a yellow oil. LCMS (method D): 1.40 min, m/z: 378.1 [M+H]⁺.

Intermediate F11: tert-butyl 3-{3-[4-(4,4,5,5-tetramethyl-1,3,2-dioxaborolan-2-yl)-1H-pyrazol-1-yl]pyrrolidin-1-yl}propanoate

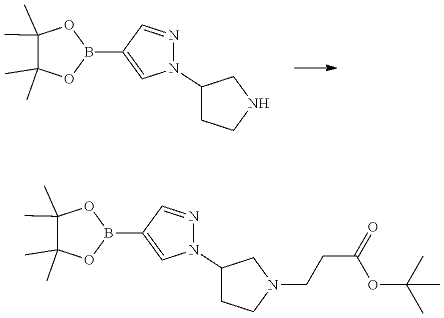 [SMILES: CC1(C)OB(c2cnn(C3CCNC3)c2)OC1(C)C ; CC(C)(C)OC(=O)CCN1CCC(n2cc(B3OC(C)(C)C(C)(C)O3)cn2)C1]

### Step 1: tert-butyl 3-{3-[4-(4,4,5,5-tetramethyl-1,3,2-dioxaborolan-2-yl)-1H-pyrazol-1-yl]pyrrolidin-1-yl}propanoate

A mixture of 1-(pyrrolidin-3-yl)-4-(4,4,5,5-tetramethyl-1,3,2-dioxaborolan-2-yl)-1H-pyrazole (280 mg, 1.06 mmol) and tert-butyl prop-2-enoate (271 mg, 2.12 mmol) in MeOH (8 mL) was stirred at RT overnight. The reaction was concentrated afford the title product (380 mg, 92%) as a yellow oil. LCMS (method D): 1.21 min, m/z: 392.0 [M+H]⁺.

### Intermediate F12: tert-butyl 2-(2-{3-[4-(4,4,5,5-tetramethyl-1,3,2-dioxaborolan-2-yl)-1H-pyrazol-1-yl]pyrrolidin-1-yl}ethoxy)acetate

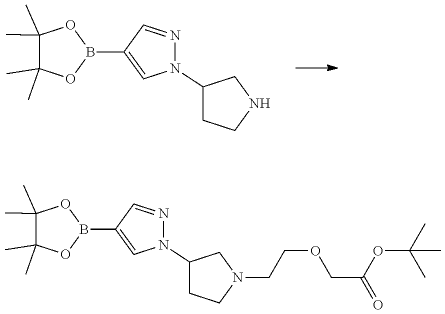 [SMILES: CC1(C)OB(c2cnn(C3CCNC3)c2)OC1(C)C ; CC(C)(C)OC(=O)COCCN1CCC(n2cc(B3OC(C)(C)C(C)(C)O3)cn2)C1]

### Step 1: tert-butyl 2-(2-{3-[4-(4,4,5,5-tetramethyl-1,3,2-dioxaborolan-2-yl)-1H-pyrazol-1-yl]pyrrolidin-1-yl}ethoxy)acetate

To a solution of 1-(pyrrolidin-3-yl)-4-(4,4,5,5-tetramethyl-1,3,2-dioxaborolan-2-yl)-1H-pyrazole (549 mg, 2.09 mmol) in MeCN (10 mL) was added K₂CO₃ (1.43 g, 10.4 mmol), and the mixture was stirred at RT for 20 minutes under N₂ before tert-butyl 2-(2-bromoethoxy)acetate (500 mg, 2.09 mmol) was added. The reaction was stirred at RT for 2 h under N₂, Then filtered. The filtrate was concentrated to afford the title compound (510 mg, 58%) as a white oil. LCMS (method D): 1.14 min, m/z: 422.1 [M+H]⁺.

### Intermediate F13: tert-butyl 3-{3-[4-(4,4,5,5-tetramethyl-1,3,2-dioxaborolan-2-yl)-1H-pyrazol-1-yl]piperidin-1-yl}propanoate

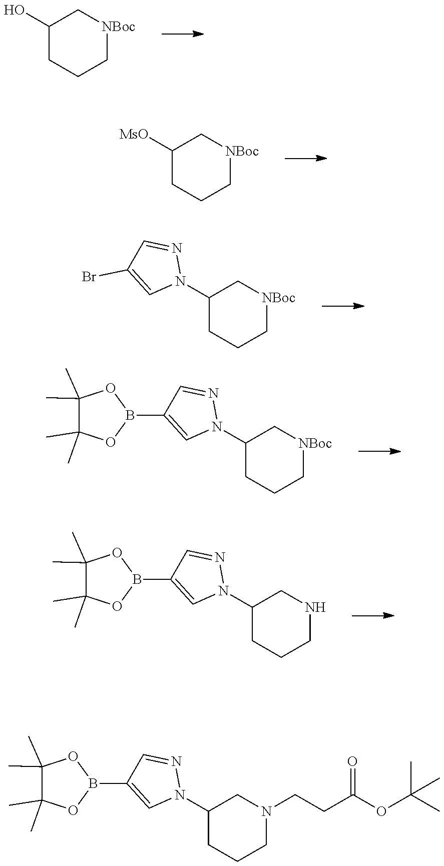 [SMILES: CC(C)(C)OC(=O)N1CCCC(O)C1 ; CC(C)(C)OC(=O)N1CCCC(OS(C)(=O)=O)C1 ; CC(C)(C)OC(=O)N1CCCC(n2cc(Br)cn2)C1 ; CC(C)(C)OC(=O)N1CCCC(n2cc(B3OC(C)(C)C(C)(C)O3)cn2)C1 ; CC1(C)OB(c2cnn(C3CCCNC3)c2)OC1(C)C ; CC(C)(C)OC(=O)CCN1CCCC(n2cc(B3OC(C)(C)C(C)(C)O3)cn2)C1]

### Step 1: tert-butyl 3-(methanesulfonyloxy)piperidine-1-carboxylate

To a solution of tert-butyl 3-hydroxypiperidine-1-carboxylate (5 g, 24.8 mmol) in DCM (30 mL) were added MsCl (4.26 g, 37.2 mmol) and Et₃N (5.01 g, 49.6 mmol), and the resulting mixture was stirred at 0° C. for 1 h. The mixture was concentrated, and the residue was diluted with DCM. The organic fraction was washed with water, dried (Na₂SO₄) and concentrated to afford the title product (6.90 g, 99%) as a yellow oil.

### Step 2: tert-butyl 3-(4-bromo-1H-pyrazol-1-yl)piperidine-1-carboxylate

A mixture of tert-butyl 3-(methanesulfonyloxy)piperidine-1-carboxylate (6.75 g, 24.1 mmol), 4-bromo-1H-pyrazole (3.54 g, 24.1 mmol) and NaH (1.71 g, 43.3 mmol) in DMF (60 mL) was stirred at 100° C. for 16 h. The mixture was concentrated, and the residue was taken up in DCM. The organic fraction was washed with water, dried (Na₂SO₄) and concentrated. The residue was purified by column chromatography (EtOAc/PE=1/5, v/v) to afford the title product (2.50 g, 31%) as a yellow oil. ¹H NMR (400 MHz, MeOD-d₄): 7.82 (s, 1H), 7.49 (s, 1H), 4.24-4.13 (m, 1H), 3.86 (bs, 1H), 3.86 (bs, 1H), 3.04-2.98 (m, 1H), 2.14-2.09 (m, 2H), 1.80 (bs, 1H), 1.63-1.56 (m, 1H), 1.45 (s, 9H).

### Step 3: 4-(4,4,5,5-tetramethyl-1,3,2-dioxaborolan-2-yl)-1H-pyrazole

A mixture of tert-butyl 3-(4-bromo-1H-pyrazol-1-yl)piperidine-1-carboxylate (2.8 g, 8.47 mmol), bis(pinacolato)diboron (2.56 g, 10.1 mmol), AcOK (2.49 g, 25.4 mmol) and Pd(dppf)Cl₂ (619 mg, 847 μmol) in 1,4-dioxane (15 mL) was stirred at 100° C. for 3 h under N₂. The mixture was concentrated and the residue was purified by column chromatography (EtOAc/PE=1/5, v/v) to afford the title product (2.50 g, 78%) as a yellow oil.

### Step 4: 3-[4-(4,4,5,5-tetramethyl-1,3,2-dioxaborolan-2-yl)-1H-pyrazol-1-yl]piperidine

To a solution of 4-(4,4,5,5-tetramethyl-1,3,2-dioxaborolan-2-yl)-1H-pyrazole (0.3 g, 795 μmol) in DCM (3 mL) was added 4M HCl in dioxane (2.27 mL, 9.08 mmol), and the reaction was stirred at RT for 1 h. The mixture was concentrated to afford the title compound (200 mg, 96%) as a yellow oil, which was used directly in the next step. LCMS (method B): 0.72 min, m/z: 278.0 [M+H]⁺.

### Step 5: tert-butyl 3-{3-[4-(4,4,5,5-tetramethyl-1,3,2-dioxaborolan-2-yl)-1H-pyrazol-1-yl]piperidin-1-yl}propanoate

To a solution of 3-[4-(4,4,5,5-tetramethyl-1,3,2-dioxaborolan-2-yl)-1H-pyrazol-1-yl]piperidine (0.2 g, 721 μmol) in MeOH (3 mL) was added tert-butyl prop-2-enoate (208 μL, 1.44 mmol), and the reaction was stirred at RT for 16 h. The reaction was concentrated to afford the title product (280 mg, 96%) as a yellow oil. LCMS (method B): 0.94 min, m/z: 406.4 [M+H]⁺. ¹H NMR (400 MHz, DMSO-d₆): 7.95 (s, 1H), 7.66 (s, 1H), 4.35 (t, 1H), 3.08 (d, 1H), 2.83 (d, 1H), 2.72 (q, 2H), 2.43 (m, 3H), 2.19 (t, 1H), 2.08 (t, 1H), 1.83 (m, 2H), 1.68 (m, 1H), 1.44 (s, 9H), 1.30 (s, 12H).

### Intermediate F14: tert-butyl 2-(2-{3-[4-(4,4,5,5-tetramethyl-1,3,2-dioxaborolan-2-yl)-1H-pyrazol-1-yl]piperidin-1-yl}ethoxy)acetate

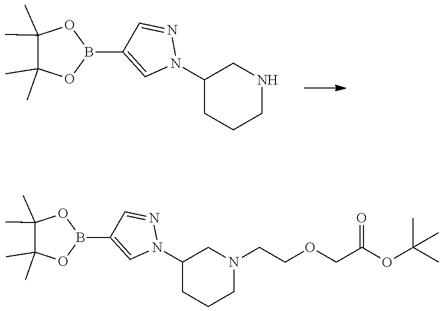 [SMILES: CC1(C)OB(c2cnn(C3CCCNC3)c2)OC1(C)C ; CC(C)(C)OC(=O)COCCN1CCCC(n2cc(B3OC(C)(C)C(C)(C)O3)cn2)C1]

### Step 1: tert-butyl 2-(2-{3-[4-(4,4,5,5-tetramethyl-1,3,2-dioxaborolan-2-yl)-1H-pyrazol-1-yl]piperidin-1-yl}ethoxy)acetate

To a solution of 3-[4-(4,4,5,5-tetramethyl-1,3,2-dioxaborolan-2-yl)-1H-pyrazol-1-yl]piperidine (0.22 g, 793 μmol) in MeCN were added K₂CO₃ (327 mg, 2.37 mmol) and tert-butyl 2-(2-bromoethoxy)acetate (189 mg, 793 μmol), and the reaction was stirred at RT for 16 h. The mixture was diluted with water and extracted with DCM. The organic phase was dried (Na₂SO₄) and concentrated afforded (300 mg, 87%) as yellow oil.

### Intermediate F15: tert-butyl 2-{4-[4-(4,4,5,5-tetramethyl-1,3,2-dioxaborolan-2-yl)-1H-pyrazol-1-yl]piperidin-1-yl}acetate

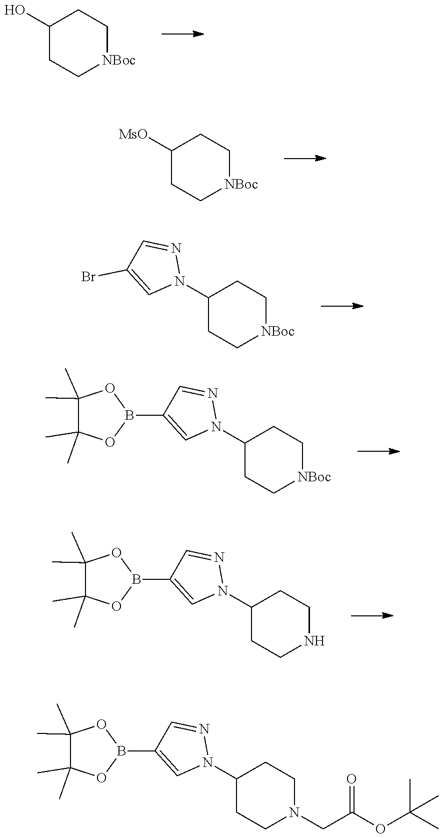 [SMILES: CC(C)(C)OC(=O)N1CCC(O)CC1 ; CC(C)(C)OC(=O)N1CCC(OS(C)(=O)=O)CC1 ; CC(C)(C)OC(=O)N1CCC(n2cc(Br)cn2)CC1 ; CC(C)(C)OC(=O)N1CCC(n2cc(B3OC(C)(C)C(C)(C)O3)cn2)CC1 ; CC1(C)OB(c2cnn(C3CCNCC3)c2)OC1(C)C ; CC(C)(C)OC(=O)CN1CCC(n2cc(B3OC(C)(C)C(C)(C)O3)cn2)CC1]

### Step 1: tert-butyl 4-(methanesulfonyloxy)piperidine-1-carboxylate

A mixture of tert-butyl 4-hydroxypiperidine-1-carboxylate (15 g, 74.5 mmol), MsCl (10.2 g, 89.3 mmol) and Et₃N (11.2 g, 111 mmol) in DCM (200 mL) was stirred at RT for 2 h. The mixture was poured into water (200 mL) and extracted with DCM (50 mL×3). The combined organic phases were dried (Na₂SO₄) and concentrated to afford the title product (19.5 g, 94%) as a yellow solid. LCMS (method A): 3.38 min, m/z: 302.1 [M+Na]⁺.

### Step 2: tert-butyl 4-(4-bromo-1H-pyrazol-1-yl)piperidine-1-carboxylate

A mixture of 4-bromo-1H-pyrazole (5 g, 34.0 mmol), tert-butyl 4-(methanesulfonyloxy) piperidine-1-carboxylate (10.4 g, 37.4 mmol) and Cs₂CO₃ (22.1 g, 68.0 mmol) in DMF (100 mL) was stirred at 100° C. under N₂ overnight. The mixture was poured into water (40 mL) and extracted with EtOAc (40 mL×3). The combined organic phases were dried (Na₂SO₄) and concentrated. The residue was purified by column chromatography (PE/EtOAc=5/1) to afford the title product (9 g, 80%) as a yellow oil. ¹H NMR (400 MHz, DMSO-d₆): 8.05 (d, J=0.8 Hz, 1H), 7.54 (d, J=0.4 Hz, 1H), 4.34 (p, J=7.2 Hz, 1H), 4.02 (d, J=13.6 Hz, 2H), 2.88 (bs, 2H), 1.99-1.95 (m, 2H), 1.76-1.72 (m, 2H), 1.41 (s, 9H).

### Step 3: tert-butyl 4-[4-(4,4,5,5-tetramethyl-1,3,2-dioxaborolan-2-yl)-1H-pyrazol-1-yl]piperidine-1-carboxylate

To a solution of tert-butyl 4-(4-bromo-1H-pyrazol-1-yl)piperidine-1-carboxylate (3.8 g, 11.5 mmol) in 1,4-dioxane (10 mL) were added AcOK (3.38 g, 34.5 mmol), Pd(dppfCl₂ (841 mg, 1.15 mmol) and bis(pinacolato)diboron (4.36 g, 17.2 mmol), and the reaction was stirred at 110° C. overnight. The mixture was concentrated, and the residue was purified by column chromatography (PE/EtOAc=5/1) to afford the title compound (2.80 g, 7.42 mmol) as an oil.

### Step 4: 4-[4-(4,4,5,5-tetramethyl-1,3,2-dioxaborolan-2-yl)-1H-pyrazol-1-yl]piperidine

To a solution of tert-butyl 4-[4-(4,4,5,5-tetramethyl-1,3,2-dioxaborolan-2-yl)-1H-pyrazol-1-yl]piperidine-1-carboxylate (3 g, 7.95 mmol) in 1,4-dioxane (15 mL) was added 4M HCl in dioxane (20 mL), and the reaction was stirred at RT for 2 h. The mixture was concentrated and the residue was adjusted to pH=8 with sat. aq. Na₂CO₃. The precipitate was collected via filtration to afford the title product (2 g, 91%) as a yellow gum. LCMS (method A): 3.81 min, m/z: 278.1 [M+H]⁺.

### Step 5: tert-butyl 2-{4-[4-(4,4,5,5-tetramethyl-1,3,2-dioxaborolan-2-yl)-1H-pyrazol-1-yl]piperidin-1-yl}acetate

To a mixture of 4-[4-(4,4,5,5-tetramethyl-1,3,2-dioxaborolan-2-yl)-1H-pyrazol-1-yl]piperidine (1.1 g, 3.96 mmol) and K₂CO₃ (2.72 g, 19.7 mmol) in MeCN (10 mL) at 0° C. was added tert-butyl 2-bromoacetate (1.15 g, 5.93 mmol), and the reaction was stirred at RT for 2 h. The mixture was concentrated, then the residue was poured into water (10 mL) and extracted with EtOAc (10 mL×3). The combined organics were dried (Na₂SO₄) and concentrated. The residue was purified by Prep-TLC (PE/EA=2/1, v/v) to afford the title product (550 mg, 36%) as a yellow oil. LCMS (method A): 2.88 min, m/z: 392.1 [M+H]⁺.

### Intermediate F16: tert-butyl 3-{4-[4-(4,4,5,5-tetramethyl-1,3,2-dioxaborolan-2-yl)-1H-pyrazol-1-yl]piperidin-1-yl}propanoate

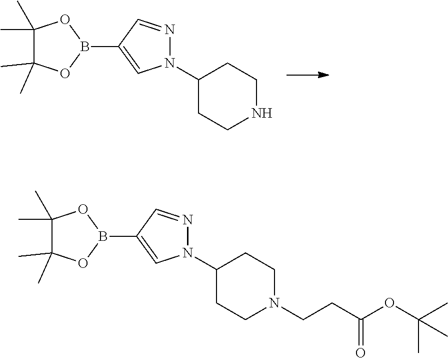 [SMILES: CC1(C)OB(c2cnn(C3CCNCC3)c2)OC1(C)C ; CC(C)(C)OC(=O)CCN1CCC(n2cc(B3OC(C)(C)C(C)(C)O3)cn2)CC1]

### Step 1: tert-butyl 3-{4-[4-(4,4,5,5-tetramethyl-1,3,2-dioxaborolan-2-yl)-1H-pyrazol-1-yl]piperidin-1-yl}propanoate

To a solution of 4-[4-(4,4,5,5-tetramethyl-1,3,2-dioxaborolan-2-yl)-1H-pyrazol-1-yl]piperidine (235 mg, 847 μmol) in MeOH (5 mL) was added tert-butyl prop-2-enoate (108 mg, 847 μmol), and the reaction was stirred at RT overnight. The mixture was concentrated to afford the title compound (340 mg, 99%) as an oil. LCMS (method B): 0.71 min, m/z: 406.0 [M+H]⁺.

### Intermediate F17: tert-butyl 2-(2-{4-[4-(4,4,5,5-tetramethyl-1,3,2-dioxaborolan-2-yl)-1H-pyrazol-1-yl]piperidin-1-yl}ethoxy)acetate

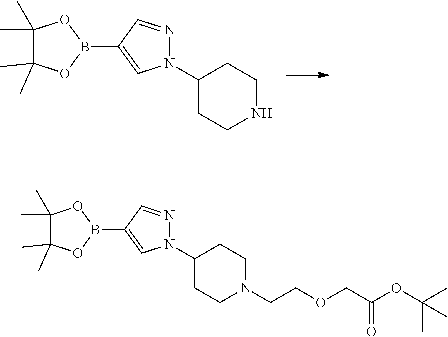 [SMILES: CC1(C)OB(c2cnn(C3CCNCC3)c2)OC1(C)C ; CC(C)(C)OC(=O)COCCN1CCC(n2cc(B3OC(C)(C)C(C)(C)O3)cn2)CC1]

### Step 1: tert-butyl 2-(2-{4-[4-(4,4,5,5-tetramethyl-1,3,2-dioxaborolan-2-yl)-1H-pyrazol-1-yl]piperidin-1-yl}ethoxy)acetate

To a solution of 4-[4-(4,4,5,5-tetramethyl-1,3,2-dioxaborolan-2-yl)-1H-pyrazol-1-yl]piperidine (231 mg, 836 μmol) in MeCN (5 mL) was added K₂CO₃ (288 mg, 2.09 mmol), and the mixture was stirred at RT for 30 min. Then, tert-butyl 2-(2-bromoethoxy)acetate (100 mg, 418 μmol) was added at 0° C., and the solution was stirred at RT under N₂ for 2 h. The mixture was extracted with EtOAc (5 mL×2), and the combined organics were washed with water (5 mL) and brine (3 mL), dried (Na₂SO₄) and concentrated. The residue was purified by reverse-phase chromatography to afford the title compound (59.0 mg, 32%) as a yellow oil. LCMS (method A): 1.23 min, m/z: 436.3 [M+H]⁺.

### Intermediate F18: tert-butyl 3-{3-[4-(4,4,5,5-tetramethyl-1,3,2-dioxaborolan-2-yl)-1H-pyrazol-1-yl]propoxy}propanoate

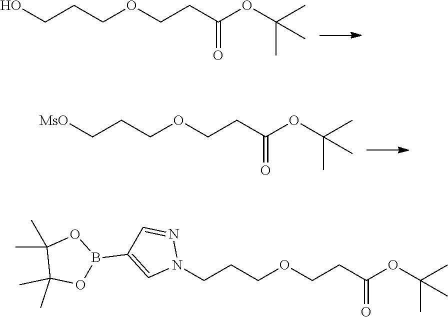

### Step 1: tert-butyl 3-[3-(methanesulfonyloxy)propoxy]propanoate

To a 0° C. mixture of tert-butyl 3-(3-hydroxypropoxy)propanoate (500 mg, 2.44 mmol) and Et₃N (987 mg, 9.76 mmol) in DCM (5 mL) was MsCl (558 mg, 4.88 mmol). The reaction was warmed to RT and stirred for 2 h. The mixture was concentrated, and the residue was poured into water (40 mL) and extracted with DCM (40 mL×3). The combined organic phases were dried over Na₂SO₄ and concentrated to afford the title product (500 mg, 58%) as a yellow oil. LCMS (method A): 3.81 min, m/z: 305.2 [M+Na]⁺.

### Step 2: tert-butyl 3-{3-[4-(4,4,5,5-tetramethyl-1,3,2-dioxaborolan-2-yl)-1H-pyrazol-1-yl]propoxy}propanoate

A mixture of 4-(4,4,5,5-tetramethyl-1,3,2-dioxaborolan-2-yl)-1H-pyrazole (300 mg, 1.54 mmol), tert-butyl 3-[3-(methanesulfonyloxy)propoxy]propanoate (519 mg, 1.84 mmol) and Cs₂CO₃ (1 g, 3.08 mmol) in MeCN (10 mL) was stirred at 70° C. under N₂ overnight. The mixture was poured into water (40 mL) and extracted with EtOAc (40 mL×3). The combined organic phases were dried over Na₂SO₄ and concentrated. The residue was purified by reverse-phase chromatography to afford the title product (400 mg, 68%) as a colourless oil. LCMS (method A): 3.74 min, m/z: 381.2 [M+H]⁺.

### Intermediate F19: tert-butyl 2-{4-[4-(4,4,5,5-tetramethyl-1,3,2-dioxaborolan-2-yl)-1H-pyrazol-1-yl]butoxy}acetate

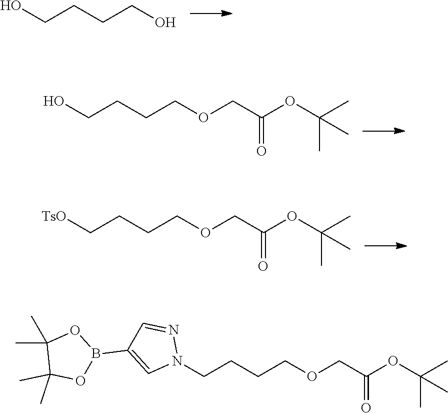

### Step 1: tert-butyl 2-(4-hydroxybutoxy)acetate

To a solution of tert-butyl 2-bromoacetate (2.0 g, 10.2 mmol) MeCN (10 mL) were added butane-1,4-diol (1.09 g, 12.2 mmol) and Cs₂CO₃ (9.93 g, 30.5 mmol), and the reaction was stirred overnight at RT overnight. The mixture was concentrated, and the residue was diluted with water (500 mL) and extracted with EtOAc (3×500 mL). The combined organics were dried over Na₂SO₄ and concentrated to afford the title product (3.00 g, >100%) as a colourless oil.

### Step 2: tert-butyl 2-{4-[(4-methylbenzenesulfonyl)oxy]butoxy}acetate

To a solution of tert-butyl 2-(4-hydroxybutoxy)acetate (3.0 g, 14.6 mmol) in DCm (20 mL) were added Et3N (4.43 g, 43.8 mmol) and TsCl (3.05 g, 16.0 mmol), and the reaction was stirred at RT overnight. The mixture was concentrated, and the residue was diluted with water (500 mL) and extracted with EtOAc (3×500 mL). The combined organics were dried over Na2SO4 and concentrated to afford the title product (2.50 g, 48%) as a colourless oil.

### Step 3: tert-butyl 2-{4-[4-(4,4,5,5-tetramethyl-1,3,2-dioxaborolan-2-yl)-1H-pyrazol-1-yl]butoxy}acetate

To a solution of 4-(4,4,5,5-tetramethyl-1,3,2-dioxaborolan-2-yl)-1H-pyrazole (40 mg, 185 μmol) in MeCN (10 mL) were added tert-butyl 2-{4-[(4-methylbenzenesulfonyl)oxy]butoxy}acetate (86.0 mg, 240 μmol) and Cs₂CO₃ (180 mg, 555 μmol), and the reaction was stirred at 80° C. for 16 hours. The mixture was filtered and concentrated to afford the title product (30 mg, 43%) as a colourless oil. LCMS (method A): 2.90 min, m/z: 381.0 [M+H]⁺.

### Intermediate F20: tert-butyl 6-[4-(4,4,5,5-tetramethyl-1,3,2-dioxaborolan-2-yl)phenoxy]hexanoate

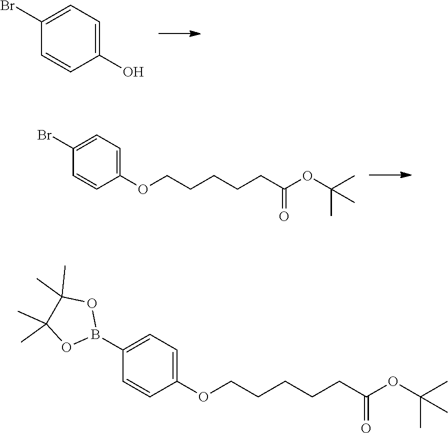

### Step 1: tert-butyl 6-(4-bromophenoxy)hexanoate

A mixture of 4-bromophenol (500 mg, 2.89 mmol), tert-butyl 6-bromohexanoate (869 mg, 3.46 mmol), and Cs₂CO₃ (1.88 g, 5.78 mmol) in DMF (10 mL) was stirred at 80° C. under N₂ for 16 h. The mixture was poured into water (100 mL) and extracted with EtOAc (50 mL×2). The combined organic phases were dried over Na₂SO₄ and concentrated. The residue was purified by column chromatography to afford the title product (545 mg, 55%) as a white oil.

### Step 2: tert-butyl 6-[4-(4,4,5,5-tetramethyl-1,3,2-dioxaborolan-2-yl)phenoxy]hexanoate

A mixture of tert-butyl 6-(4-bromophenoxy)hexanoate (200 mg, 582 μmol), bis(pinacolato)diboron (221 mg, 872 μmol), AcOK (170 mg, 1.74 mmol) and Pd(PPh₃)₂Cl₂ (40.8 mg, 58.2 μmol) in dioxane (5 ml) was stirred at 100° C. under N₂ for 16 h. The mixture was poured into water (100 mL) and extracted with EtOAc (50 mL×2). The combined organic phases were dried over Na₂SO₄ and concentrated. The residue was purified by prep-TLC (DCM:MeOH=20:1) to afford the title product (93 mg, 41%) as a yellow oil.

### Intermediate F21: tert-butyl 2-{2-[4-(4,4,5,5-tetramethyl-1,3,2-dioxaborolan-2-yl)phenoxy]ethoxy}acetate

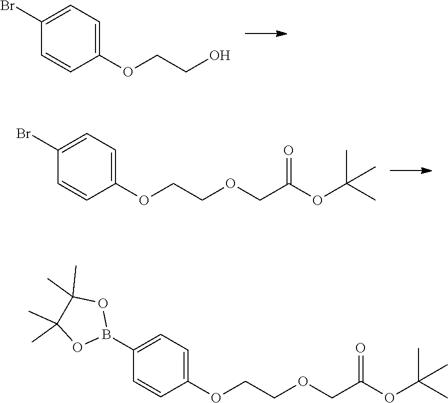

### Step 1: tert-butyl 2-[2-(4-bromophenoxy)ethoxy]acetate

A mixture of 2-(4-bromophenoxy)ethan-1-ol (2.0 g, 9.21 mmol), tert-butyl 2-bromoacetate (2.69 g, 13.8 mmol) and Cs₂CO₃ (8.99 g, 27.6 mmol) in MeCN (40 mL) was stirred at 100° C. for 16 h. The mixture was poured into water (100 mL) and extracted with EtOAc (50 mL×2). The combined organic phases were dried over Na2SO4 and concentrated. The residue was purified by column chromatography to afford the title product (2.2 g, 72%) as a colourless oil.

### Step 2: tert-butyl 2-{2-[4-(4,4,5,5-tetramethyl-1,3,2-dioxaborolan-2-yl)phenoxy]ethoxy}acetate

A mixture of tert-butyl 2-[2-(4-bromophenoxy)ethoxy]acetate (1.5 g, 4.52 mmol), bis(pinacolato)diboron (1.49 g, 5.87 mmol) Pd(OAc)₂ (50.7 mg, 226 μmol) and AcOK (1.32 g, 13.5 mmol) in dioxane (20 mL) was stirred 80° C. overnight. The mixture was poured into water (100 mL) and extracted with EtOAc (50 mL×2). The combined organic phases were dried over Na2SO4 and concentrated. The residue was purified by prep-TLC (DCM:MeOH=20:1, v/v) to afford the title product (800 mg, 2.11 mmol) as a colourless oil.

### Intermediate F22: tert-butyl 6-{[5-(4,4,5,5-tetramethyl-1,3,2-dioxaborolan-2-yl)pyridin-2-yl]oxy}hexanoate

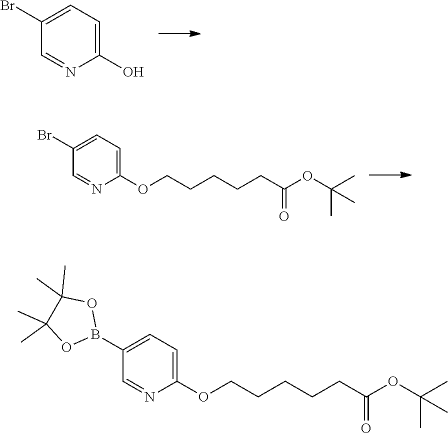

### Step 1: tert-butyl 6-[(5-bromopyridin-2-yl)oxy]hexanoate

A mixture of tert-butyl 6-bromohexanoate (500 mg, 1.99 mmol), Cs₂CO₃ (537 mg, 1.65 mmol) and 5-bromopyridin-2-ol (287 mg, 1.65 mmol) in MeCN (10 mL) was stirred at 80° C. under N₂ overnight. The mixture was diluted with H₂O (100 mL) and extracted with EtOAc (3×200 mL). The combined organic layers were dried (Na₂SO₄) and concentrated, and the crude residue was purified by column chromatography (PE:EtOAc, 2:1) to afford the title product (512 mg, 90%) as a white solid. LCMS (method A): 3.56 min; m/z: 366.0 [M+H]⁺.

### Step 2: tert-butyl 6-{[5-(4,4,5,5-tetramethyl-1,3,2-dioxaborolan-2-yl)pyridin-2-yl]oxy}hexanoate

To a solution of tert-butyl 6-[(5-bromopyridin-2-yl)oxy]hexanoate (250 mg, 726 μmol) in dioxane (5 mL) were added bis(pinacolato)diboron (274 mg, 1.08 mmol) Pd(dppf)Cl₂—CH₂Cl₂ (59.2 mg, 72.6 μmol) and AcOK (142 mg, 1.45 mmol), and the mixture was stirred at 100° C. under N₂ overnight. The mixture was diluted with H₂O (100 mL) extracted with EtOAc (3×200 mL). The combined organic layers were dried (Na₂SO₄) and concentrated, and the residue was purified by column chromatography (PE:EtOAc, 2:1) to afford the title product (70.0 mg, 25%) as a white oil. LCMS (method A): 3.96 min, m/z: 392.2 [M+H]⁺.

### Intermediate F23: ethyl 9-[4-(4,4,5,5-tetramethyl-1,3,2-dioxaborolan-2-yl)-1H-pyrazol-1-yl]nonanoate

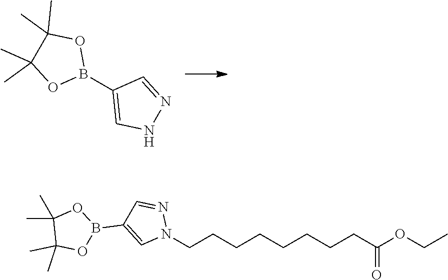 [SMILES: CC1(C)OB(c2cn[nH]c2)OC1(C)C ; CCOC(=O)CCCCCCCCn1cc(B2OC(C)(C)C(C)(C)O2)cn1]

### Step 1: ethyl 9-[4-(4,4,5,5-tetramethyl-1,3,2-dioxaborolan-2-yl)-1H-pyrazol-1-yl]nonanoate

A mixture of ethyl 9-bromononanoate (1.5 g, 5.65 mmol), 4-(4,4,5,5-tetramethyl-1,3,2-dioxaborolan-2-yl)-1H-pyrazole (995 mg, 5.13 mmol) and Cs₂CO₃ (5.01 g, 15.4 mmol) in MeCN (25 mL) was stirred at 80° C. for 16 h. The mixture was concentrated and the residue was purified by column chromatography (DCM/MeOH=200/1) to afford the title compound (1.50 g, 90%) as a yellow solid. LCMS (Method A): 4.09 min. m/z: 379.3 [M+H]⁺.

### Synthesis of Further Compounds

### Compound 1001: (2S,4R)-1-[(2S)-2-{3-[4-(4-amino-3-{4-ethanesulfonamido-3-[(4-fluorophenyl)methoxy]phenyl}-1H-pyrazolo[4,3-c]pyridin-7-yl)-1H-pyrazol-1-yl]propanamido}-3,3-dimethylbutanoyl]-4-hydroxy-N-{[4-(4-methyl-1,3-thiazol-5-yl)phenyl]methyl}pyrrolidine-2-carboxamide

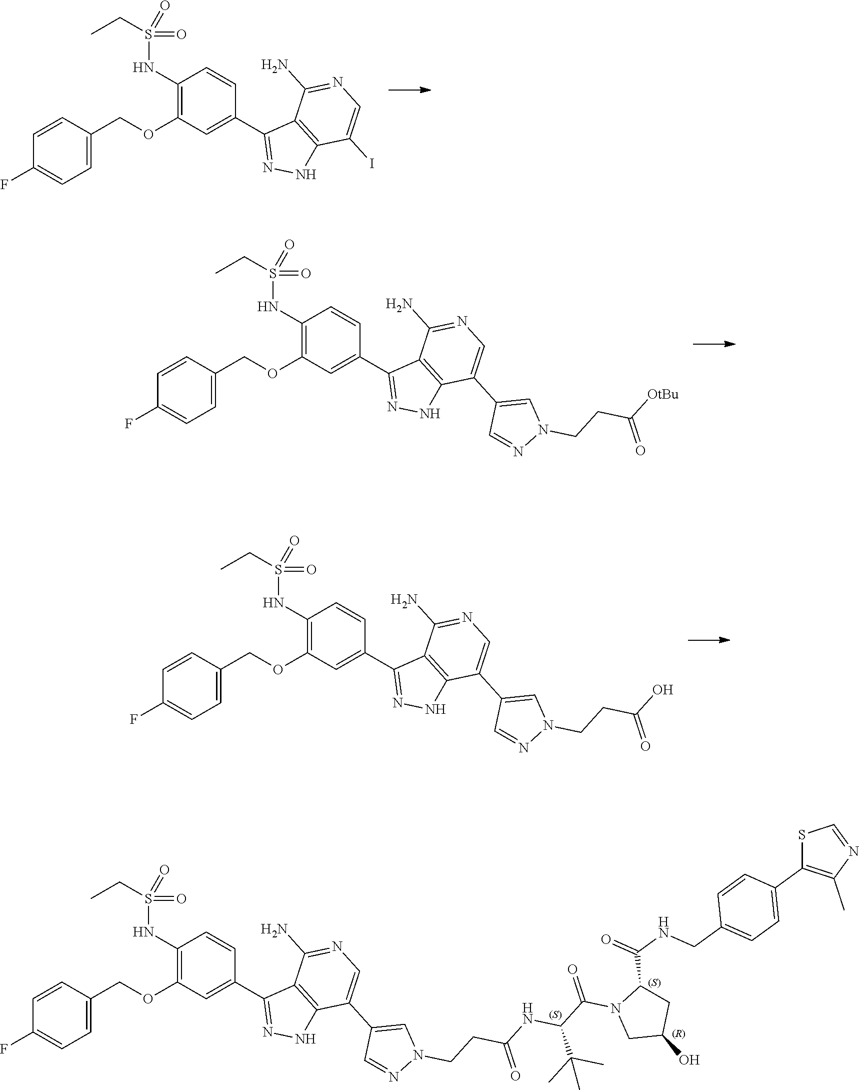

### Step 1: tert-butyl 3-[4-(4-amino-3-{4-ethanesulfonamido-3-[(4-fluorophenyl)methoxy]phenyl}-1H-pyrazolo[4,3-c]pyridin-7-yl)-1H-pyrazol-1-yl]propanoate

A mixture of N-(4-{4-amino-7-iodo-1H-pyrazolo[4,3-c]pyridin-3-yl}-2-[(4-fluorophenyl) methoxy]phenyl)ethane-1-sulfonamide (351 mg, 620 μmol), tert-butyl 3-[4-(4,4,5,5-tetramethyl-1,3,2-dioxaborolan-2-yl)-1H-pyrazol-1-yl]propanoate (200 mg, 620 μmol), Na₂CO₃ (153 mg, 1.24 mmol) and Pd(dppf)Cl₂ (50.6 mg, 62.0 μmol) in 1,4-dioxane (8 mL) and H₂O (2 mL) was stirred under N₂ at 100° C. with μW irradiation for 1 h. The mixture was concentrated and the residue was poured into water (100 mL), then extracted with EtOAc (50 mL×2). The combined organics were dried (Na₂SO₄) and concentrated. The residue was purified by column chromatography (DCM/MeOH=20:1) to afford the title product (270 mg, 69%) as a brown solid. LCMS (method A): 3.10 min, m/z: 636.3 [M+H]⁺. ¹H NMR (400 MHz, DMSO-d₆) 9.16 (s, 1H), 8.28 (s, 1H), 7.96 (d, J=12.4 Hz, 2H), 7.65-7.61 (m, 2H), 7.48 (d, J=8.0 Hz, 1H), 7.43 (s, 1H), 7.25 (t, J=9.2 Hz, 3H), 6.30 (s, 2H), 5.23 (s, 2H), 4.37 (t, J=6.8 Hz, 2H), 3.07 (q, J=7.2 Hz, 2H), 2.85 (t, J=6.8 Hz, 2H), 1.38 (s, 9H), 1.18 (t, J=7.2 Hz, 3H).

### Step 2: 3-[4-(4-amino-3-{4-ethanesulfonamido-3-[(4-fluorophenyl)methoxy]phenyl}-1H-pyrazolo[4,3-c]pyridin-7-yl)-1H-pyrazol-1-yl]propanoic acid

A solution of tert-butyl 3-[4-(4-amino-3-{4-ethanesulfonamido-3-[(4-fluorophenyl)methoxy]phenyl}-1H-pyrazolo[4,3-c]pyridin-7-yl)-1H-pyrazol-1-yl]propanoate (148 mg, 0.23 mmol) in DCM/TFA (1.16 mL, 3/1) was stirred at RT. After 16 h, the reaction mixture was concentrated to afford the TFA salt of the title product (156 mg, 96%) as a beige solid. LCMS (method C): 1.61 min, m/z: 580.2 [M+H]⁺.

### Step 3: (2S,4R)-1-[(2S)-2-{3-[4-(4-amino-3-{4-ethanesulfonamido-3-[(4-fluorophenyl) methoxy]phenyl}-1H-pyrazolo[4,3-c]pyridin-7-yl)-1H-pyrazol-1-yl]propanamido}-3,3-dimethylbutanoyl]-4-hydroxy-N-{[4-(4-methyl-1,3-thiazol-5-yl)phenyl]methyl}pyrrolidine-2-carboxamide

A mixture of 3-[4-(4-amino-3-{4-ethanesulfonamido-3-[(4-fluorophenyl)methoxy]phenyl}-1H-pyrazolo[4,3-c]pyridin-7-yl)-1H-pyrazol-1-yl]propanoic acid (26 mg, 44.9 μmol), (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide (20.2 mg, 47.1 μmol), HATU (22.1 mg, 58.3 μmol) and DIPEA (23.3 μL, 0.13 mmol) in DMF (448 μL) was stirred at RT. After 16 h, the mixture was concentrated and the residue was purified by preparative-HPLC (Method C) to afford the title compound (11.8 mg, 26%) as a pale brown powder. LCMS (method C): 1.76 min, m/z: 993.4 [M+H]⁺, 496.9 [M+2H]²⁺.

The following examples were similarly prepared from Intermediate A2B19 and the appropriate Intermediate F according to the procedure for the synthesis of Compound 1001.

TABLE 10
Compounds 1002 to 1005

| Compound | Structure | Compound Name and Analytical Data |
|---|---|---|
| 1002 | 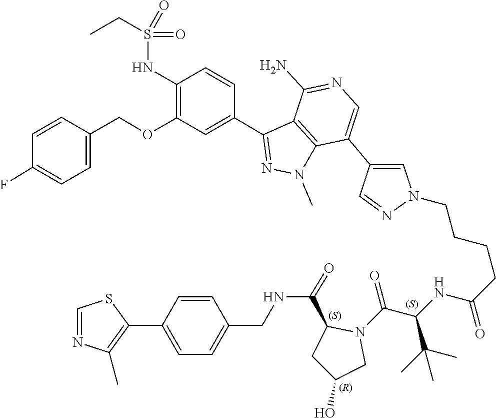 | (2S,4R)-1-[(2S)-2-{5-[4-(4-amino-3-{4- ethanesulfonamido-3-[(4- fluorophenyl)methoxy]phenyl}-1H- pyrazolo[4,3-c]pyridin-7-yl)-1H-pyrazol-1- yl]pentanamido}-3,3-dimethylbutanoyl]-4- hydroxy-N-{[4-(4-methyl-1,3-thiazol-5- yl)phenyl]methyl}pyrrolidine-2-carboxamide From Intermediate F2. LCMS (method D): 1.80 min, m/z: 510.8 [M + 2H]²⁺. |
| 1003 | 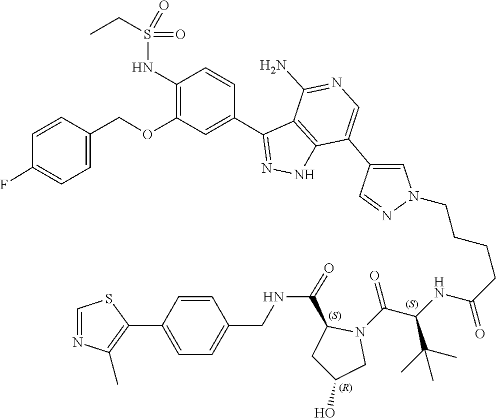 | (2S,4R)-1-[(2S)-2-{7-[4-(4-amino-3-{4- ethanesulfonamido-3-[(4- fluorophenyl)methoxy]phenyl}-1H- pyrazolo[4,3-c]pyridin-7-yl)-1H-pyrazol-1- yl]heptanamido}-3,3-dimethylbutanoyl]-4- hydroxy-N-{[4-(4-methyl-1,3-thiazol-5- yl)phenyl]methyl}pyrrolidine-2-carboxamide From Intermediate F3. LCMS (method D): 1.86 min, m/z: 1049.4 [M + H]⁺, 525.2 [M + 2H]²⁺. |
| 1004 | 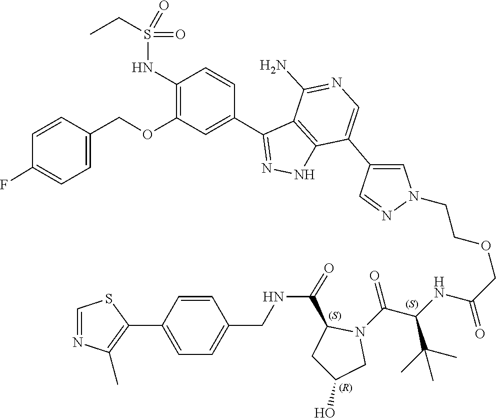 | (2S,4R)-1-[(2S)-2-(2-{2-[4-(4-amino-3-{4- ethanesulfonamido-3-[(4- fluorophenyl)methoxy]phenyl}-1H- pyrazolo[4,3-c]pyridin-7-yl)-1H-pyrazol-1- yl]ethoxy}acetamido)-3,3-dimethylbutanoyl]-4- hydroxy-N-{[4-(4-methyl-1,3-thiazol-5- yl)phenyl]methyl}pyrrolidine-2-carboxamide From Intermediate F6. LCMS (method D): 1.75 min, m/z: 1022.2 [M + H]⁺, 512.2 [M + 2H]²⁺. |
| 1005 | 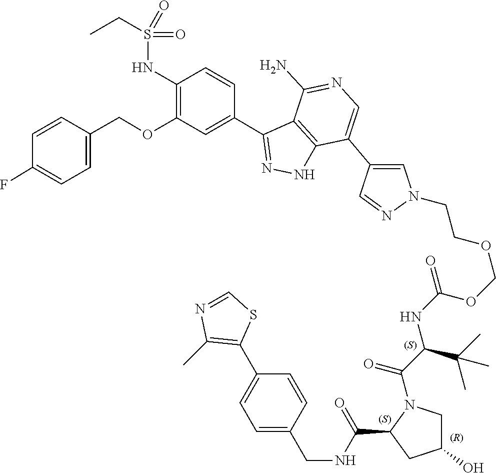 | (2S,4R)-1-[(2S)-2-[2-(2-{2-[4-(4-amino-3-{4- ethanesulfonamido-3-[(4- fluorophenyl)methoxy]phenyl}-1H- pyrazolo[4,3-c]pyridin-7-yl)-1H-pyrazol-1- yl]ethoxy}ethoxy)acetamido]-3,3- dimethylbutanoyl]-4-hydroxy-N-{[4-(4-methyl- 1,3-thiazol-5-yl)phenyl]methyl}pyrrolidine-2- carboxamide From Intermediate F7. LCMS (method D): 1.78 min, m/z: 1067.2 [M + H]⁺, 534.2 [M + 2H]²⁺. |

### Compound 1006: (2S,4R)-1-[(2S)-2-(2-{2-[2-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1-yl)ethoxy]ethoxy}acetamido)-3,3-dimethylbutanoyl]-4-hydroxy-N-{[4-(4-methyl-1,3-thiazol-5-yl)phenyl]methyl}pyrrolidine-2-carboxamide

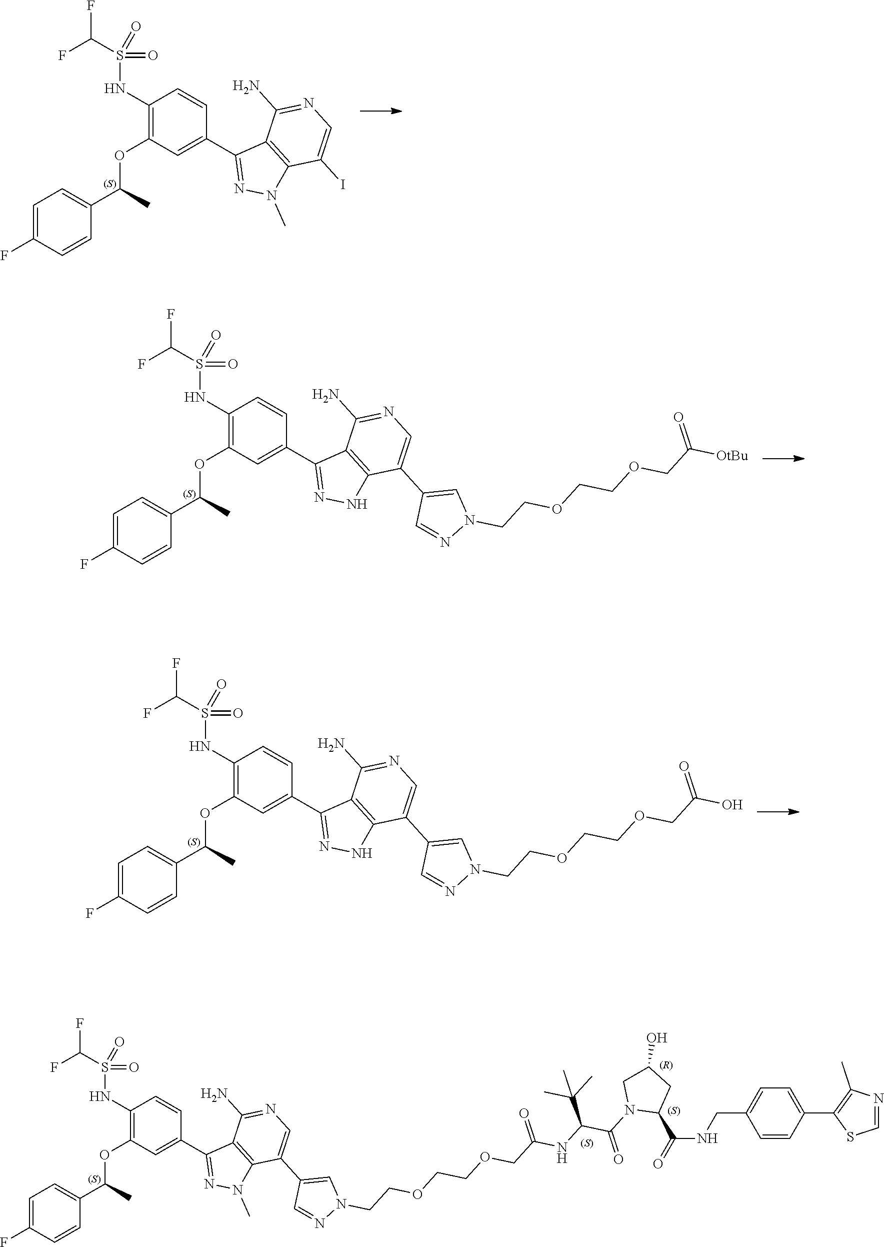

### Step 1: tert-butyl 2-{2-[2-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)ethoxy]ethoxy}acetate

To a mixture of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (200 mg, 323 μmol), tert-butyl 2-(2-{2-[4-(4,4,5,5-tetramethyl-1,3,2-dioxaborolan-2-yl)-1H-pyrazol-1-yl]ethoxy}ethoxy) acetate (141 mg, 356 μmol) and Na₂CO₃ (68.5 mg, 647 μmol) in 1,4-Dioxane (16 mL) and water (4 mL) was added Pd(dppf)Cl₂ (52.8 mg, 64.7 μmol), and the reaction was heated at 100° C. under N₂ for 40 h. The mixture was concentrated, and the residue partitioned between EtOAc (25 mL) and water (25 mL). The layers were separated, and the aqueous phase was extracted with EtOAc (2×25 mL). The combined organics were washed with brine, dried (MgSO₄) and concentrated. The residue was purified by flash chromatography (0-5% MeOH:DCM) to afford the title compound (129 mg, 53%) as a yellow solid. LCMS (method F): 1.97 min, m/z: 760.2 [M+H]⁺. ¹H NMR (300 MHz, CDCl₃): 7.85 (bs, 1H), 7.74 (d, J=7.7 Hz, 1H), 7.60 (s, 1H), 7.40 (bs, 1H), 7.31 (dd, J=6.9 Hz, 3H), 7.14 (d, J=8.2 Hz, 1H), 7.06 (dd, J=8.0 Hz, 2H), 6.96 (s, 1H), 6.37 (t, J=53.4 Hz, 1H), 5.45 (bs, 1H), 4.44 (s, 2H), 3.96 (s, 2H). 3.91 (s. 2H). 3.81 (s. 3H). 3.66 (s. 4H). 1.72 (d. J=1.6 Hz. 3H). 1.39 (s. 9H).

### Step 2: 2-{2-[2-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)ethoxy]ethoxy}acetic acid

A solution of tert-butyl 2-{2-[2-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)ethoxy]ethoxy}acetate (125 mg, 165 μmol) in HCO₂H (5 mL) was stirred at RT for 1 h. The mixture was concentrated and the residual formic acid removed by lyophilisation to afford the title product (90.6 mg, 79%) as a light tan solid. LCMS (method F): 1.59 min, m/z: 704.2 [M+H]⁺. ¹H NMR (300 MHz, DMSO-d₆): 7.93 (s, 1H), 7.63-7.54 (m, 4H), 7.41 (d, J=7.9 Hz, 1H), 7.19-7.12 (m 4H), 7.02 (t, J=52.6 Hz, 1H), 5.89 (bs, 2H), 5.65 (q, J=6.7 Hz, 1H), 4.32 (t, J=5.2 Hz, 2H), 3.99 (s, 2H), 3.82 (t, J=5.2 Hz, 2H), 3.69 (s, 3H), 3.55 (s, 4H), 1.58 (d, J=6.7 Hz, 3H).

### Step 3: (2S,4R)-1-[(2S)-2-(2-{2-[2-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl) ethoxy]ethoxy}acetamido)-3,3-dimethylbutanoyl]-4-hydroxy-N-{[4-(4-methyl-1,3-thiazol-5-yl) phenyl]methyl}pyrrolidine-2-carboxamide

To a solution of 2-{2-[2-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)ethoxy]ethoxy}acetic acid (50 mg, 71 μmol) in dry DMF (354 μL) were added DIPEA (37.0 μL, 213 μmol), HATU (35.0 mg, 92.3 μmol) and (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide hydrochloride (36.4 mg, 78.1 μmol), sequentially. Then, the reaction was stirred at RT under N₂ for 21 h. The mixture was concentrated and the residue was purified by flash chromatography (0-20% (10% NH₃ in MeOH):DCM), followed by further purification by prep-HPLC to afford the title compound (18.5 mg, 23%) as a white solid. LCMS (method G): 1.81 min, m/z 1117.2 [M+H]⁺. ¹H NMR (300 MHz, DMSO-d₆): 8.96 (s, 1H), 8.58 (t, J=6.0 Hz, 1H), 7.97 (s, 1H), 7.63 (s, 1H), 7.60-7.52 (m, 3H), 7.47-7.40 (m, 2H), 7.37 (s, 4H), 7.23-7.08 (m, 3H), 6.94 (t, J=52.6 Hz, 1H), 6.59 (s, 2H), 5.63 (q, J=6.2 Hz, 1H), 5.16 (s, 1H), 4.56 (d, J=9.6 Hz, 1H), 4.47-4.31 (m, 4H), 4.23 (dd, J=15.8, 5.6 Hz, 1H), 4.10 (s, 1H), 3.95 (s, 2H), 3.85 (t, J=5.5 Hz, 2H), 3.70 (s, 3H), 3.66-3.54 (m, 5H), 3.17 (s, 4H), 2.42 (s, 2H), 2.12-1.99 (m, 1H), 1.89 (ddd, J=13.0, 8.8, 4.4 Hz, 1H), 1.58 (d, J=6.3 Hz, 3H), 0.91 (s, 9H).

### Compound 1007: (2S,4S)-1-[(2S)-2-(2-{2-[2-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)ethoxy]ethoxy}acetamido)-3,3-dimethylbutanoyl]-4-hydroxy-N-{[4-(4-methyl-1,3-thiazol-5-yl)phenyl]methyl}pyrrolidine-2-carboxamide

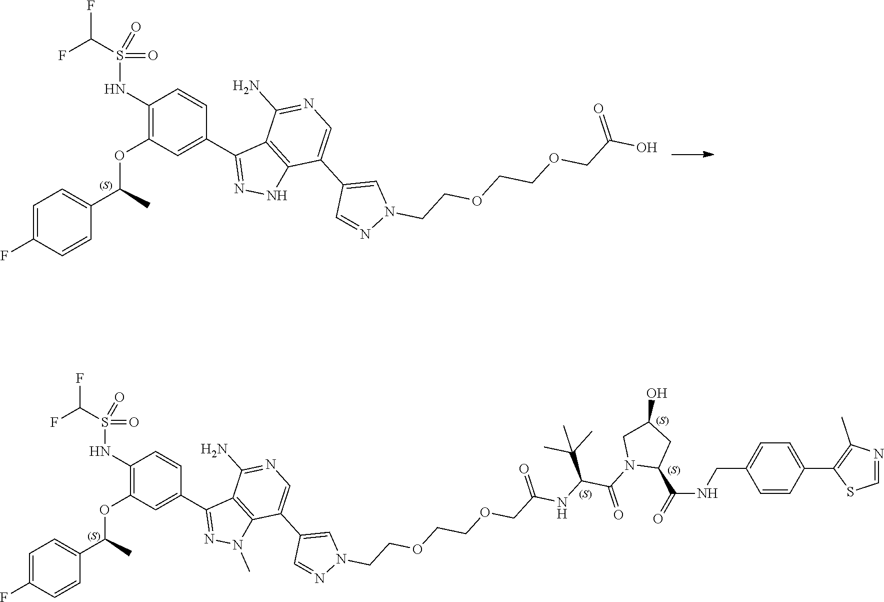

### Step 1: (2S,4S)-1-[(2S)-2-(2-{2-[2-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)ethoxy]ethoxy}acetamido)-3,3-dimethylbutanoyl]-4-hydroxy-N-{[4-(4-methyl-1,3-thiazol-5-yl)phenyl]methyl}pyrrolidine-2-carboxamide

To a solution of 2-{2-[2-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)ethoxy]ethoxy}acetic acid (39 mg, 55.4 μmol) in dry DMF (276 μL) were added DIPEA (28.8 μL, 166 μmol), HATU (27.3 mg, 72.0 μmol) and (2S,4S)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide hydrochloride (28.4 mg, 60.9 μmol), sequentially. Then, the reaction was stirred at RT under N₂ for 21 h. The mixture was concentrated and the residue was purified by flash chromatography (0-20% (10% NH₃ in MeOH):DCM), followed by further purification by prep-HPLC to afford the title compound (10.9 mg, 18%) as an off-white solid. LCMS (method G): 1.91 min, m/z: 1117.2 [M+H]⁺. ¹H NMR (300 MHz, DMSO-d₆): 8.95 (s, 1H), 8.62 (t, J=6.0 Hz, 1H), 8.02 (d, J=0.8 Hz, 1H), 7.67 (d, J=0.8 Hz, 1H), 7.61-7.52 (m, 2H), 7.48-7.38 (m, 2H), 7.37 (s, 4H), 7.22-7.10 (m, 3H), 6.93 (t, J=52.6 Hz, 1H), 5.62 (q, J=6.2 Hz, 1H), 5.49 (bs, 1H), 4.50 (d, J=9.2 Hz, 3H), 4.45-4.22 (m, 6H), 3.94 (s, 2H), 3.85 (t, J=5.0 Hz, 4H), 3.72 (s, 6H), 2.42 (s, 3H), 2.34 (td, J=8.4, 4.3 Hz, 1H), 1.59 (d, J=6.3 Hz, 3H), 0.91 (s, 9H).

### Compound 1008: (2S,4R)-1-[(2S)-2-[7-(4-{4-amino-3-[4-(difluoromethanesulfonamido)-3-[(1 S)-1-(4-fluorophenyl)ethoxy]phenyl]-1-methyl-1H-pyrazolo[4,3-c]pyridin-7-yl}-1H-pyrazol-1-yl)heptanamido]-3,3-dimethylbutanoyl]-4-hydroxy-N-{[4-(4-methyl-1,3-thiazol-5-yl)phenyl]methyl}pyrrolidine-2-carboxamide

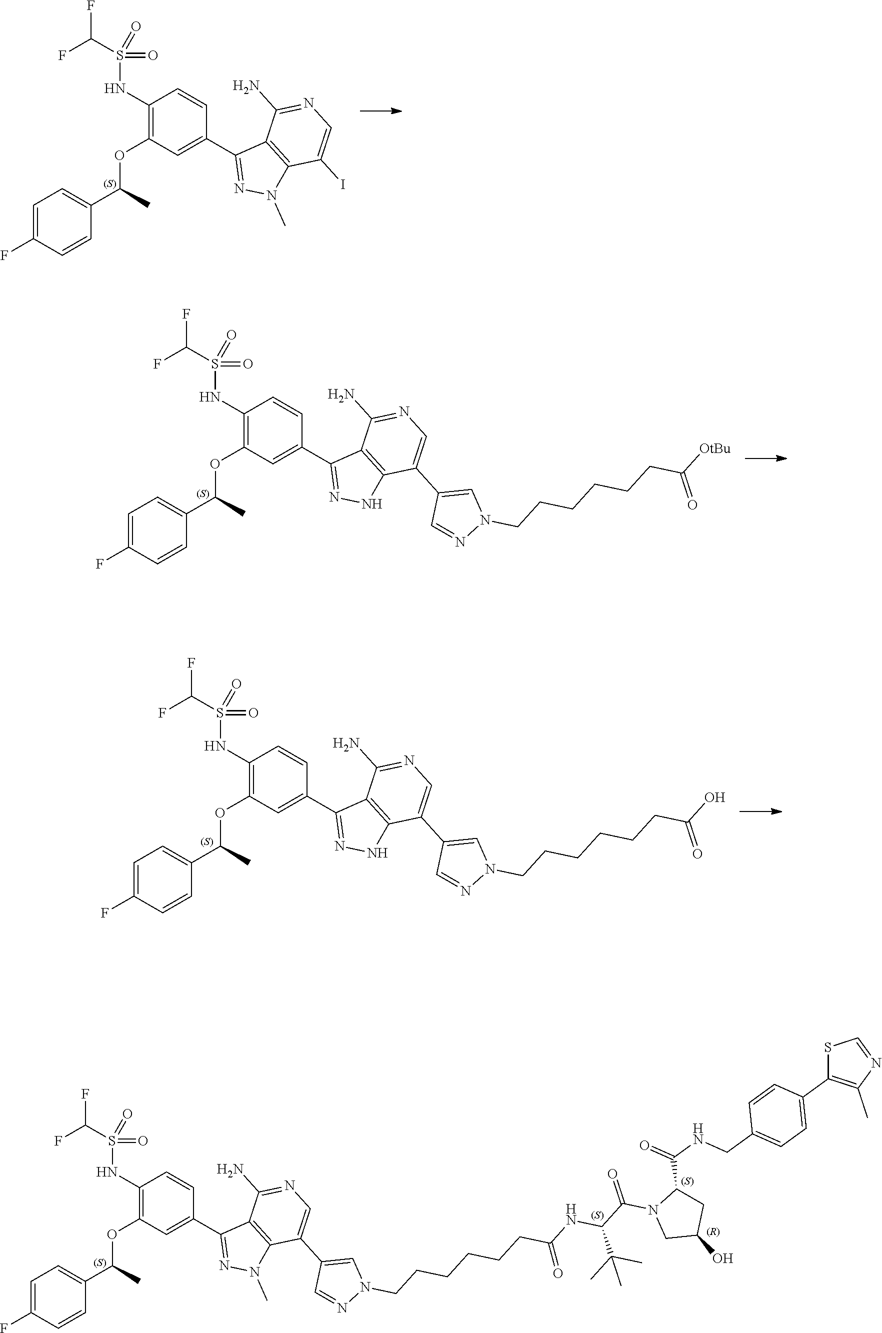

### Step 1: tert-butyl 7-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)heptanoate

To a mixture of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (200 mg, 324 μmol), tert-butyl 7-[4-(4,4,5,5-tetramethyl-1,3,2-dioxaborolan-2-yl)-1H-pyrazol-1-yl]heptanoate (134 mg, 356 μmol) and Na₂CO₃ (68.5 mg, 647 μmol) in DME (5 mL) and water (1 mL) was added Pd(PPh₃)₄ (37.3 mg, 32.3 μmol), and the reaction was heated at 80° C. under N₂ for 22 h. Once cooled, the mixture was concentrated and the residue was partitioned between EtOAc and water. The layers were separated and the aqueous phase was extracted twice with EtOAc. The combined organics were dried (MgSO₄) and concentrated. The residue was purified by flash chromatography to afford the title product (116 mg, 49%) as a yellow glass. LCMS (method F): 2.33 min, m/z: 742.8 [M+H]⁺. ¹H NMR (300 MHz, CDCl₃): 7.67 (d, J=8.2 Hz, 1H), 7.56 (s, 1H), 7.55 (d, J=0.8 Hz, 1H), 7.45 (d, J=0.8 Hz, 1H), 7.37-7.27 (m, 2H), 7.20 (dd, J=8.2, 1.8 Hz, 1H), 7.08-6.97 (m, 3H), 6.35 (t, J=53.6 Hz, 1H), 5.46 (q, J=6.3 Hz, 1H), 4.19 (t, J=7.1 Hz, 2H), 3.77 (s, 2H), 2.21 (t, J=7.4 Hz, 2H), 1.99-1.88 (m, 2H), 1.68 (d, J=6.4 Hz, 3H), 1.65-1.51 (m, 2H), 1.41-1.33 (m, 2H), 1.24 (s, 9H).

### Step 2: 7-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)heptanoic acid

A solution of tert-butyl 7-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl-}1H-pyrazol-1-yl) heptanoate (110 mg, 148 μmol) in HCO₂H (5 mL) was stirred at RT for 2 h. The mixture was concentrated and the residual formic acid removed by lyophilisation to afford the title product (94.3 mg, 93%) as a white solid. LCMS (method F): 1.79 min, m/z: 686.8 [M+H]⁺.

### Step 3: (2S,4R)-1-[(2S)-2-[7-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl) heptanamido]-3,3-dimethylbutanoyl]-4-hydroxy-N-{[4-(4-methyl-1,3-thiazol-5-yl)phenyl]methyl}pyrrolidine-2-carboxamide

To a solution of 7-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)heptanoic acid (90 mg, 131 μmol) in dry DMF (654 μL) were added DIPEA (68.3 μL, 393 μmol), HATU (64.6 mg, 170 μmol) and (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide hydrochloride (67.2 mg, 144 μmol), sequentially. Then, the reaction was stirred at RT under an atmosphere of nitrogen for 21 h. The mixture was concentrated and the residue was purified by flash chromatography (0-20% (10% NH3 in MeOH):DCM), followed by further purification by prep-HPLC to afford the title compound (37.4 mg, 26%) as a white solid. LCMS (method G): 1.94 min, m/z: 1099.6 [M+H]⁺. ¹H NMR (300 MHz, DMSO-d₆): 8.97 (s, 1H), 8.56 (t, J=6.0 Hz, 1H), 7.94 (d, J=0.8 Hz, 1H), 7.86 (d, J=9.4 Hz, 1H), 7.62-7.51 (m, 4H), 7.45-7.34 (m, 4H), 7.22-7.09 (m, 4H), 6.94 (t, J=52.6 Hz, 1H), 6.00 (s, 2H), 5.65 (q, J=6.1 Hz, 1H), 5.13 (s, 1H), 4.54 (d, J=9.4 Hz, 1H), 4.48-4.38 (m, 2H), 4.35 (s, 1H), 4.23 (d, J=5.4 Hz, 1H), 4.16 (q, J=6.9, 6.4 Hz, 3H), 3.71 (s, 3H), 3.69-3.63 (m, 2H), 2.44 (s, 3H), 2.29-2.19 (m, 1H), 2.19-1.97 (m, 2H), 1.95-1.84 (m, 1H), 1.81 (m, 1H), 1.58 (d, J=6.3 Hz, 3H), 1.48 (d, J=7.2 Hz, 2H), 1.27 (s, 4H), 0.93 (s, 9H).

The following examples were similarly prepared from Intermediate A1B1 and the appropriate Intermediate F according to the procedure for the synthesis of Compound 1008.

TABLE 11
Compounds 10009 to 1018

| Example | | |
|---|---|---|
| No | Structure | Compound Name and Analytical Data |
| 1009 | 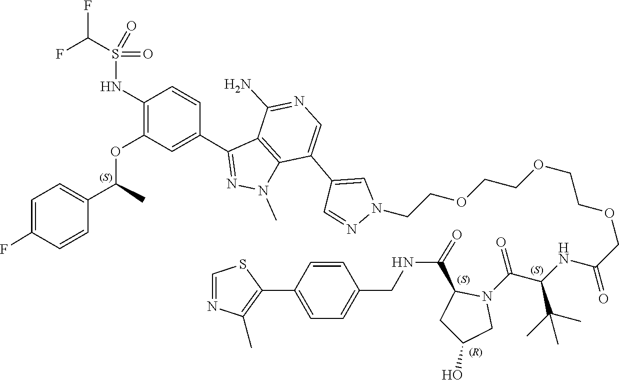 | (2S,4R)-1-[(2S)-2-[2-(2-{2-[2-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1- yl)ethoxy]ethoxy}ethoxy)acetamido]-3,3-dimethylbutanoyl]-4-hydroxy-N-{[4-(4-methyl-1,3- thiazol-5-yl)phenyl]methyl}pyrrolidine-2-carboxamide From Intermediate F8. LCMS (method G): 1.86 min, m/z: 1161.6 [M + H]⁺. ¹H NMR (300 MHz, DMSO-d₆): 8.97 (s, 1H), 8.58 (t, J = 6.0 Hz, 1H), 7.98 (d, J = 0.8 Hz, 1H), 7.66 (d, J = 0.7 Hz, 1H), 7.63-7.52 (m, 3H), 7.47-7.40 (m, 2H), 7.38 (s, 4H), 7.24-7.09 (m, 4H), 6.95 (t, J = 52.6 Hz, 1H), 6.85 (s, 2H), 5.64 (q, J = 6.3 Hz, 1H), 5.16 (s, 1H), 4.55 (d, J = 9.6 Hz, 1H), 4.49-4.17 (m, 6H), 3.92 (s, 2H), 3.79 (t, J = 5.3 Hz, 2H), 3.72 (s, 3H), 3.68-3.60 (m, 2H), 3.60-3.49 (m, 8H), 2.43 (s, 3H), 2.12-1.99 (m, 1H), 1.97-1.82 (m, 1H), 1.59 (d, J = 6.2 Hz, 3H), 0.92 (s, 9H). |
| 1010 | 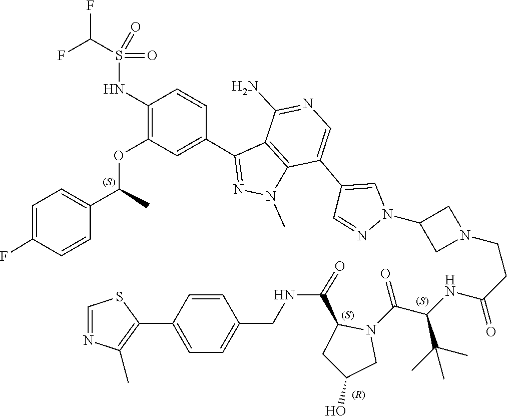 | (2S,4R)-1-[(2S)-2-{3-[3-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1-yl) azetidin-1-yl]propanamido}-3,3-dimethylbutanoyl]-4-hydroxy-N-{[4-(4-methyl-1,3-thiazol- 5-yl)phenyl]methyl}pyrrolidine-2-carboxamide From Intermediate F9. LCMS (method G): 1.46 min, m/z: 1098.6 [M + H]⁺. ¹H NMR (300 MHz, DMSO-d₆): 8.97 (s, 1H), 8.58 (t, J = 6.3 Hz, 1H), 8.16 (s, 1H), 7.95 (s, 1H) 7.59-7.54 (m, 2 H), 7.46-7.36 (m, 6H), 7.20-7.14 (m, 2H), 7.10 (d, J = 1.8 Hz, 1H), 7.02 (t, J = 52.6 Hz, 1H), 5.62, (q, J = 6.2 Hz, 1H), 5.45-5.39 (m, 1H), 4.65 (bs, 1H), 4.57 (d, J = 9.3 Hz, 2H), 4.48-4.36 (m, 5H), 4.21 (dd, J = 15.8, 5.4 Hz, 1H), 3.75 (s, 3H), 2.70-2.61 (m, 2H), 2.44 (s, 3H), 2.06-2.02 (m, 1H), 1.96-1.87 (m, 1H) 1.59 (d, J = 6.2 Hz, 3H), 0.96 (s, 9H). [Four protons not observed] |
| 1011 | 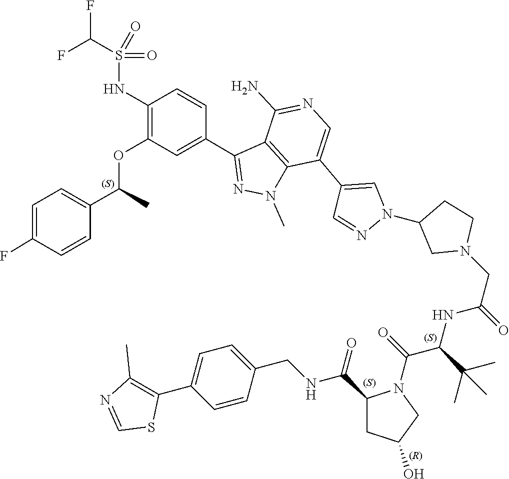 | (2S,4R)-1-[(2S)-2-{2-[3-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1- yl)pyrrolidin-1-yl]acetamido}-3,3-dimethylbutanoyl]-4-hydroxy-N-{[4-(4-methyl-1,3-thiazol-5- yl)phenyl]methyl}pyrrolidine-2-carboxamide From Intermediate F10. LCMS (method G): 1.56 min, m/z: 1098.2 [M + H]⁺. ¹H NMR (300 MHz, DMSO-d₆): 8.92 (d, J = 9.8 Hz, 1 H), 8.59 (q, J = 6.3 Hz, 1H), 8.13 (s, 1 H), 8.01 (d, J = 7.3 Hz, 1H), 7.81 (dd, J = 23.1, 9.8 Hz, 1H), 7.60-7.51 (m, 3H), 7.40-7.32 (m, 4H), 7.17-7.07 (m, 3H), 6.95 (t, J = 52.8 Hz, 1H), 5.69 (bs, 1H), 5.62 (q, J = 6.4 Hz, 1H), 5.17 (bs, 1H), 5.01 (bs, 1H), 4.52 (d, J = 9.5 Hz, 1H), 4.45-4.33 (m, 2H), 4.28-4.17 (m, 2H), 3.66 (d, J = 3.8 Hz, 3H),3.55 (s, 2H), 2.72-2.65 (m, 2H), 2.48 (s, 3H), 2.23-2.17 (m, 2H), 2.08-2.00 (m, 1H), 1.93-1.89 (m, 1H), 1.57 (d, J = 6.2 Hz, 3H), 0.91 (d, J = 12.5 Hz, 9H). |
| 1012 | 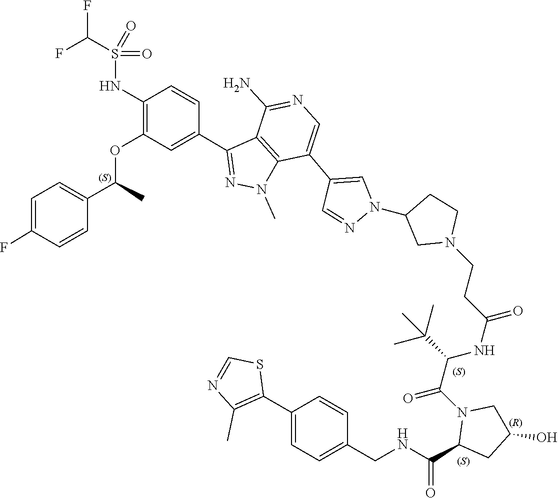 | (2S,4R)-1-[(2S)-2-{3-[3-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1- yl)pyrrolidin-1-yl]propanamido}-3,3-dimethylbutanoyl]-4-hydroxy-N-{[4-(4-methyl-1,3-thiazol- 5-yl)phenyl]methyl}pyrrolidine-2-carboxamide From Intermediate F11. LCMS (method G): 1.58 min, m/z: 1110.0 [M − H]⁻. ¹H NMR (300 MHz, DMSO-d₆): 10.60 (s, 1H), 9.89 (d, J = 44.3 Hz, 2H), 9.00 (s, 1H), 8.58 (t, J = 6.1 Hz, 1H), 8.36 (d, J = 9.3 Hz, 1H), 8.18 (s, 1H), 7.86 (s, 1H), 7.58 (dt, J = 8.7, 2.7 Hz, 3H), 7.50-7.34 (m, 6H), 7.26-7.14 (m, 3H), 7.12 (d, J = 1.8 Hz, 1H), 7.05 (t, J = 52.5 Hz, 1H), 5.64 (q, J = 6.3 Hz, 1H), 5.33 (d, J = 26.8 Hz, 2H), 5.16 (s, 1H), 4.59 (d, J = 9.3 Hz, 1H), 4.44 (td, J = 14.7, 12.8, 5.5 Hz, 3H), 4.22 (dd, J = 15.9, 5.4 Hz, 2H), 3.76 (s, 3H), 3.70 (dd, J = 7.1, 3.5 Hz, 2H), 3.61 (d, J = 10.7 Hz, 2H), 3.48 (s, 1H), 2.80-2.74 (m, 3H), 2.45 (s, 3H), 2.09 (s, 2H), 2.04 (d, J = 8.3 Hz, 1H), 1.93 (ddd, J = 13.1, 8.8, 4.5 Hz, 1H), 1.60 (d, J = 6.3 Hz, 3H), 0.97 (s, 9H). |
| 1013 | 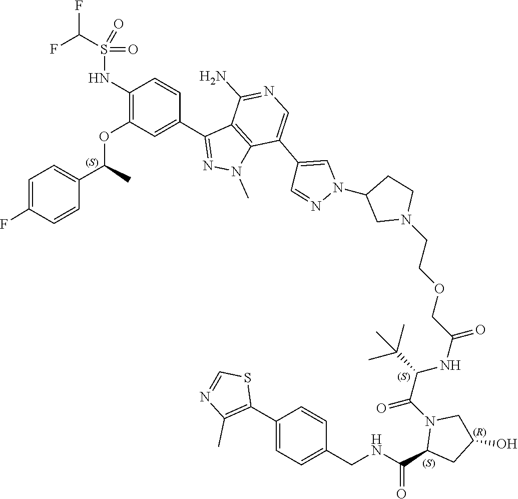 | (2S,4R)-1-[(2S)-2-(2-{2-[3-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1- yl)pyrrolidin-1-yl]ethoxy}acetamido)-3,3-dimethylbutanoyl]-4-hydroxy-N-{[4-(4-methyl-1,3- thiazol-5-yl)phenyl]methyl}pyrrolidine-2-carboxamide From Intermediate F12. LCMS: rt 1.59 min, m/z 1142.2 [M + H]⁺. |
| 1014 | 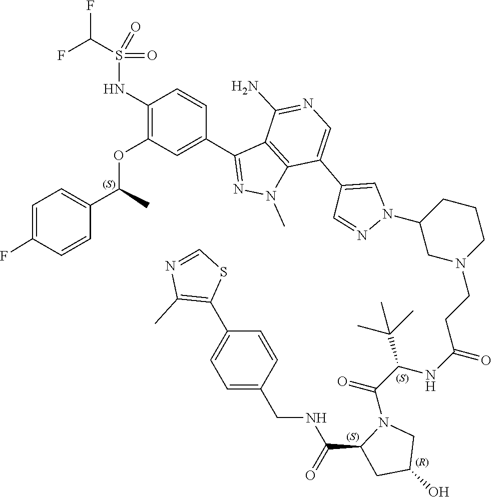 | (2S,4R)-1-[(2S)-2-{3-[3-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1-yl) piperidin-1-yl]propanamido}-3,3-dimethylbutanoyl]-4-hydroxy-N-{[4-(4-methyl-1,3-thiazol- 5-yl)phenyl]methyl}pyrrolidine-2-carboxamide From Intermediate F13. LCMS (method G): 1.61 min, m/z: 563.8 [M + 2H]²⁺. ¹H NMR (300 MHz, DMSO-d₆): 8.96 (s, 1H), 8.57 (t, J = 5.9 Hz, 1H), 8.46-8.32 (m, 1H), 8.13 (s, 1H), 7.98 (d, J = 6.7 Hz, 1H), 7.62-7.48 (m, 4H), 7.47-7.31 (m, 5H), 7.22-7.01 (m, 5H), 6.53 (s, 1H), 5.66-5.63 (m, 3H), 5.13 (s, 1H), 4.60-4.50 (m, 4H), 4.45-4.40 (m, 2H), 4.34 (s, 1H), 4.19 (s, 2H), 3.67 (s, 3H), 3.61 (s, 2H), 2.76 (s, 1H), 2.42 (s, 3H), 2.29 (s, 1H), 2.11-1.99 (m, 3H), 1.88 (ddd, J = 13.3, 8.9, 4.8 Hz, 2H), 1.73 (s, 1H), 1.56 (d, J = 6.2 Hz, 3H), 0.94 (d, J = 10.5 Hz, 9H). [2 protons not observed]. |
| 1015 | 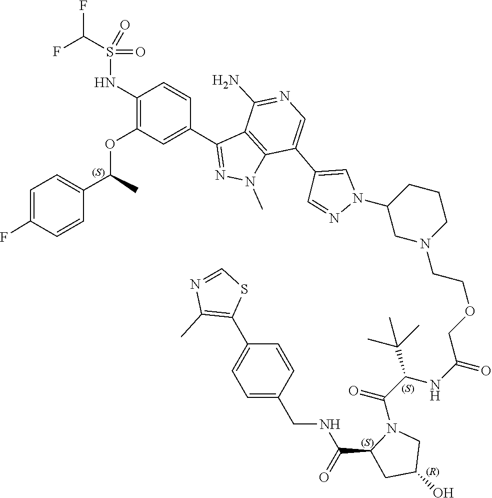 | (2S,4R)-1-[(2S)-2-(2-{2-[3-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1-yl) piperidin-1-yl]ethoxy}acetamido)-3,3-dimethylbutanoyl]-4-hydroxy-N-{[4-(4-methyl-1,3- thiazol-5-yl)phenyl]methyl}pyrrolidine-2-carboxamide From Intermediate F14. LCMS (method G): 1.58 min, m/z: 579.0 [M + 2H]²⁺. ¹H NMR (300 MHz, DMSO-d₆): 8.98 (s, 1H), 8.60 (t, J = 5.5 Hz, 1H), 8.10 (d, J = 3.5 Hz, 1H), 7.73 (s, 1H), 7.57 (td, J = 5.6, 2.6 Hz, 3H), 7.48-7.34 (m, 6H), 7.26-7.08 (m, 5H), 7.05 (t, J = 52.3 Hz, 1H), 5.65 (q, J = 6.4 Hz, 1H), 5.17 (s, 1H), 4.57 (dd, J = 9.6, 2.9 Hz, 2H), 4.42 (q, J = 7.1, 6.7 Hz, 4H), 4.34 (s, 1H), 4.23 (dd, J = 15.8, 5.4 Hz, 2H), 4.09 (s, 2H), 3.80 (s, 2H), 3.72 (d, J = 2.2 Hz, 3H), 3.60 (s, 2H), 2.44 (s, 3H), 2.15 (s, 2H), 2.06 (t, J = 10.1 Hz, 2H), 1.97-1.78 (m, 4H), 1.59 (dd, J = 6.4, 2.1 Hz, 3H), 0.94 (s, 9H). [2 protons not observed] |
| 1016 | 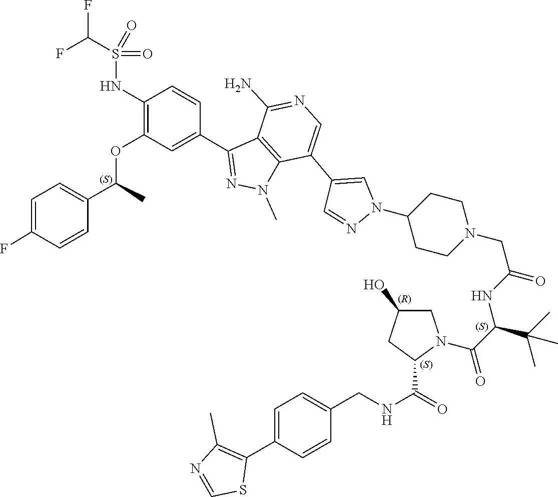 | (2S,4R)-1-[(2S)-2-{2-[4-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1-yl) piperidin-1-yl]acetamido}-3,3-dimethylbutanoyl]-4-hydroxy-N-{[4-(4-methyl-1,3-thiazol-5- yl)phenyl]methyl}pyrrolidine-2-carboxamide From Intermediate F15. LCMS (method G): 1.57 min, m/z: 1112.8 [M + H]⁺. ¹H NMR (300 MHz, DMSO-d₆): 8.94 (s, 1H), 8.60 (t, J = 6.0 Hz, 1H), 8.02 (d, J = 0.8 Hz, 1H), 7.83 (d, J = 9.5 Hz, 1H), 7.62-7.51 (m, 3H), 7.40 (d, J = 3.7 Hz, 4H), 7.24-7.12 (m, 3H), 7.10 (d, J = 2.4 Hz, 1H), 6.96 (t, J = 52.8 Hz, 1H), 5.72-5.60 (m, 3H), 5.16 (d, J = 3.4 Hz, 1H), 4.54 (d, J = 9.6 Hz, 1H), 4.50- 4.38 (m, 2H), 4.38-4.22 (m, 3H), 3.73-3.61 (m, 5H), 2.99 (d, J = 10.2 Hz, 2H), 2.43 (s, 3H), 2.09-2.07 (m, 6H), 1.91 (ddd, J = 13.0, 8.9, 4.4 Hz, 1H), 1.58 (d, J = 6.2 Hz, 3H), 0.97 (s, 9H). |
| 1017 | 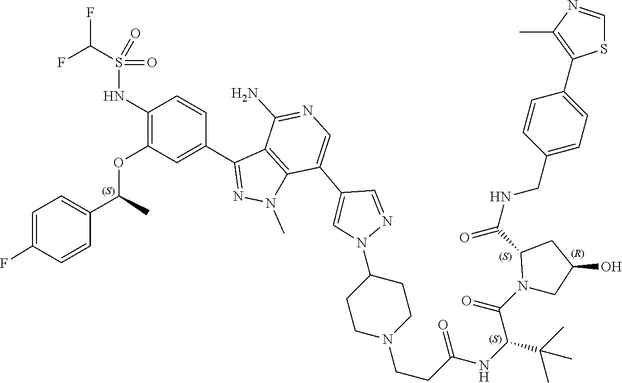 | (2S,4R)-1-[(2S)-2-{3-[4-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1-yl) piperidin-1-yl]propanamido}-3,3-dimethylbutanoyl]-4-hydroxy-N-{[4-(4-methyl-1,3-thiazol- 5-yl)phenyl]methyl}pyrrolidine-2-carboxamide From Intermediate F16. LCMS (method G): 1.56 min, m/z: 1124.2 [M − H]⁻. ¹H NMR (300 MHz, DMSO-d₆): 8.95 (s, 1H), 8.61-8.46 (m, 2H), 8.14 (s, 1H), 7.94 (s, 1H), 7.59-7.48 (m, 4H), 7.42-7.30 (m, 5H), 7.20-7.04 (m, 3H), 6.91 (t, J = 53.0 Hz, 1H), 5.72-5.59 (m, 3H), 5.16 (bs, 1H), 4.57 (d, J = 9.4 Hz, 1H), 4.44 (q, J = 7.2, 6.3 Hz, 2H), 4.36 (d, J = 6.1 Hz, 2H), 4.26 (bs, 1H), 4.17 (dd, J = 16.0, 5.5 Hz, 2H), 3.64 (s, 3H), 3.09 (d, J = 12.9 Hz, 4H), 2.71 (dt, J = 14.2, 6.2 Hz, 3H), 2.42 (s, 3H), 2.39-2.27 (m, 2H), 2.16-1.99 (m, 6H), 1.91 (ddd, J = 12.9, 8.7, 4.5 Hz, 1H), 1.57 (d, J = 6.3 Hz, 3H), 0.97 (s, 9H). |
| 1018 | 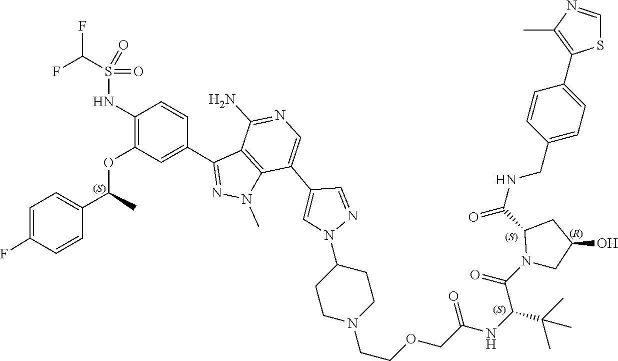 | (2S,4R)-1-[(2S)-2-(2-{2-[4-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1-yl) piperidin-1-yl]ethoxy}acetamido)-3,3-dimethylbutanoyl]-4-hydroxy-N-{[4-(4-methyl-1,3- thiazol-5-yl)phenyl]methyl}pyrrolidine-2-carboxamide From Intermediate F17. LCMS (method G): 1.57 min, m/z: 1156.2 [M + H]⁺. ¹H NMR (300 MHz, DMSO-d₆): 8.96 (s, 1H), 8.59 (t, J = 6.0 Hz, 1H), 8.14 (s, 1H), 7.96 (d, J = 0.8 Hz, 1H), 7.62-7.46 (m, 5H), 7.38 (s, 4H), 7.20-7.11 (m, 2H), 7.11-7.02 (m, 3H), 6.88 (t, J = 53.0 Hz, 1H), 5.71- 5.59 (m, 3H), 4.57 (d, J = 9.5 Hz, 1H), 4.50-4.31 (m, 4H), 4.23 (dd, J= 15.7, 5.6 Hz, 3H), 4.00 (d, J = 1.8 Hz, 2H), 3.66 (d, J = 4.5 Hz, 7H), 3.13 (s, 2H), 2.73 (s, 2H), 2.43 (s, 3H), 2.08 (d, J = 10.1 Hz, 5H), 1.90 (ddd, J = 13.0, 8.9, 4.4 Hz, 2H), 1.56 (d, J = 6.3 Hz, 3H), 0.95 (s, 9H). |
| 1040 | 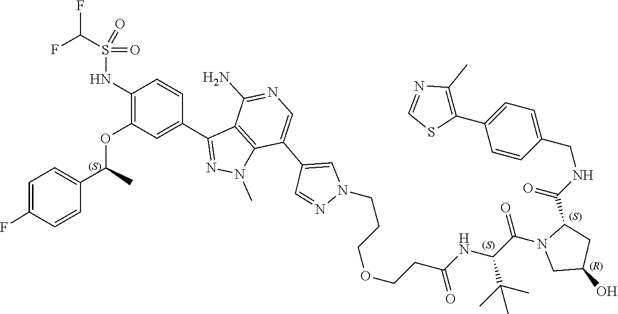 | (2S,4R)-1-[(2S)-2-{3-[3-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1- yl)propoxy]propanamido}-3,3-dimethylbutanoyl]-4-hydroxy-N-{[4-(4-methyl-1,3-thiazol- 5-yl)phenyl]methyl}pyrrolidine-2-carboxamide From Intermediate F18. LCMS (method A): 2.83 min, m/z: 550.2 [M + 2H]²⁺. ¹H NMR (400 MHz, CDCl₃): 8.65 (s, 1H), 7.67 (d, J = 0.8 Hz, 1H), 7.61 (d, J = 8.2 Hz, 1H), 7.56 (s, 1H), 7.50 (s, 1H), 7.34- 7.29 (m, 6H), 7.16 (dd, J = 8.2, 1.6 Hz, 1H), 7.05 (d, J = 1.6 Hz, 1H), 7.03-6.92 (m, 3H), 6.34 (t, J = 53.8 Hz, 1H), 5.46 (q, J = 6.3 Hz, 1H), 4.62 (t, J = 8.0 Hz, 1H), 4.56 (d, J = 8.9 Hz, 1H), 4.50 (d, J = 8.2 Hz, 2H), 4.31-4.28 (m, 2H), 4.01 (d, J = 11.3 Hz, 1H), 3.77 (s, 2H), 3.64-3.61 (m, 2H), 3.41 (t, J = 5.6 Hz, 2H), 3.16 (d, J = 7.3 Hz, 6H), 2.47 (s, 3H), 2.15 (dd, J = 11.3, 5.3 Hz, 2H), 1.65 (d, J = 6.4 Hz, 3H), 0.94 (s, 9H). |
| 1056 | 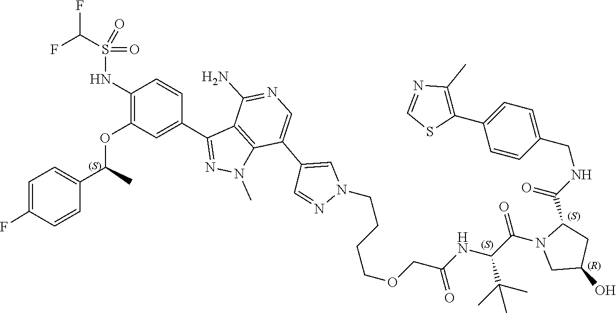 | (2S,4R)-1-[(2S)-2-{2-[4-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1- yl)butoxy]acetamido}-3,3-dimethylbutanoyl]-4-hydroxy-N-{[4-(4-methyl-1,3-thiazol-5- yl)phenyl]methyl}pyrrolidine-2-carboxamide From Intermediate F19. LCMS (method A2) 2.53 min, m/z: 1100.3 [M + H]⁺. ¹H NMR (400 MHz, DMSO-d₆): 9.12 (s, 1H), 8.95 (s, 1H), 8.62 (t, J = 6.0 Hz, 1H), 7.97 (s, 1H), 7.60 (s, 1H), 7.57 (s, 3H), 7.46-7.38 (m, 2H), 7.37 (s, 4H), 7.19-7.11 (m, 4H), 6.36 (s, 2H), 5.65 (q, J = 6.3 Hz, 1H), 5.19 (s, 1H), 4.57 (d, J = 9.6 Hz, 1H), 4.46 (t, J = 8.1 Hz, 1H), 4.41-4.31 (m, 2H), 4.26 (d, J = 5.7 Hz, 1H), 4.21 (t, J = 7.1 Hz, 2H), 4.02 (q, J = 7.1 Hz, 1H), 3.94 (d, J = 2.5 Hz, 2H), 3.71 (s, 3H), 3.61 (tt, J = 13.2, 7.0 Hz, 6H), 3.51 (t, J = 6.3 Hz, 3H), 3.12 (q, J = 7.4 Hz, 4H), 2.42 (s, 2H), 1.91 (td, J = 6.5, 5.3, 3.2 Hz, 3H), 1.59 (d, J = 6.3 Hz, 3H), 0.93 (s, 9H). |
| 1042 | 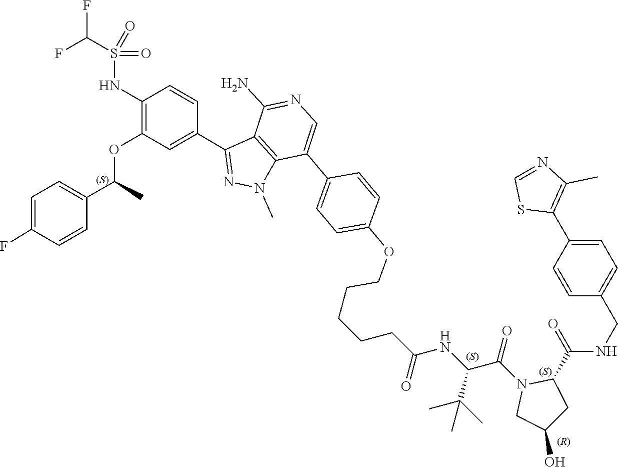 | (2S,4R)-1-[(2S)-2-[6-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}phenoxy) hexanamido]-3,3-dimethylbutanoyl]-4-hydroxy-N-{[4-(4-methyl-1,3-thiazol-5-yl) phenyl]methyl}pyrrolidine-2-carboxamide From Intermediate F20. LCMS (Method A2): 2.94 min, m/z 1110.3 [M + H]⁺. ¹H NMR (400 MHz, DMSO-d₆): 10.61 (s, 1H), 8.98 (s, 1H), 8.57 (s, 1H), 7.89 (d, J = 9.4 Hz, 1H), 7.59 (d, J = 15.6 Hz, 7H), 7.47-7.37 (m, 9H), 7.19 (d, J = 4.7 Hz, 6H), 7.10-7.03 (m, 4H), 5.65 (d, J = 6.2 Hz, 1H), 4.56 (d, J = 9.3 Hz, 1H), 4.45 (d, J = 13.2 Hz, 2H), 4.36 (s, 1H), 4.23 (s, 1H), 4.03 (d, J = 5.3 Hz, 3H), 3.69-3.65 (m, 2H), 3.58-3.56 (m, 4H), 2.82 (s, 1H), 2.55 (s, 2H), 2.46-2.43 (m, 3H), 2.30 (d, J = 20.7 Hz, 2H), 2.18 (s, 1H), 2.01 (s, 2H), 1.91 (ddd, J = 12.9, 8.6, 4.5 Hz, 1H), 1.75 (d, J = 6.8 Hz, 3H), 1.60 (d, J = 6.3 Hz, 4H), 1.55 (t, J = 5.0 Hz, 3H), 1.45 (s, 3H), 1.00-0.91 (m, 9H). |
| 1049 | 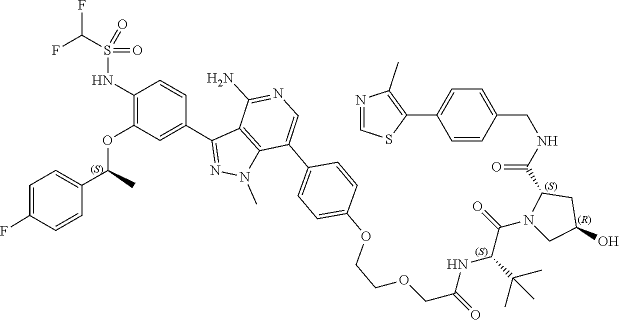 | (2S,4R)-1-[(2S)-2-{2-[2-(4-{4-amino-3-[4-(difluoromethanesulfonamido)-3-[(1S)- 1-(4-fluorophenyl)ethoxy]phenyl]-1-methyl-1H-pyrazolo[4,3-c]pyridin-7- yl}phenoxy)ethoxy]acetamido}-3,3-dimethylbutanoyl]-4-hydroxy-N-{[4-(4- methyl-1,3-thiazol-5-yl)phenyl]methyl}pyrrolidine-2-carboxamide From Intermediate F21. LCMS (Method A2) 3.17 min, m/z 550.3 [M + 2H]²⁺. ¹H NMR (400 MHz, CDCl₃): 8.66 (s, 1H), 7.71 (d, J = 8.3 Hz, 1H), 7.50 (s, 1H), 7.34 (s, 7H), 7.25-7.17 (m, 3H), 7.09-7.02 (m, 5H), 6.36 (t, J = 53.5 Hz, 1H), 5.49 (d, J = 6.4 Hz, 1H), 4.75 (t, J = 7.9 Hz, 1H), 4.55 (q, J = 8.9, 8.5 Hz, 3H), 4.34 (dd, J = 14.9, 5.2 Hz, 1H), 4.23 (t, J = 4.5 Hz, 2H), 4.16-4.08 (m, 3H), 3.94 (dd, J = 5.9, 3.2 Hz, 2H), 3.77-3.57 (m, 6H), 2.64-2.50 (m, 2H), 2.47 (s, 3H), 2.33-2.07 (m, 4H), 1.71 (d, J = 6.4 Hz, 3H), 0.97 (s, 9H). |
| 1050 | 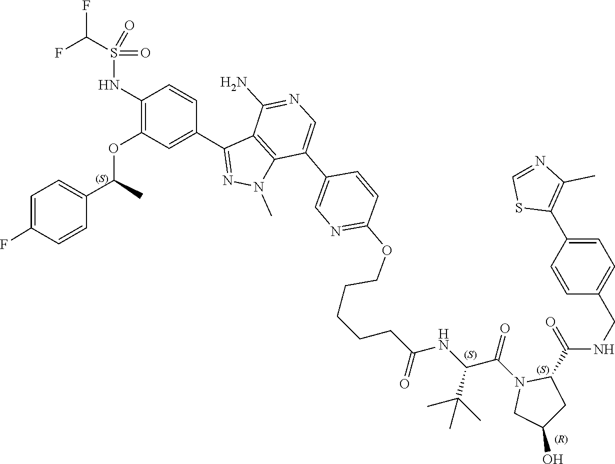 | (2S,4R)-1-[(2S)-2-{6-[(5-{4-amino-3-[4-(difluoromethanesulfonamido)-3-[(1S)- 1-(4-fluorophenyl)ethoxy]phenyl]-1-methyl-1H-pyrazolo[4,3-c]pyridin-7-yl}pyridin- 2-yl)oxy]hexanamido}-3,3-dimethylbutanoyl]-4-hydroxy-N-{[4-(4-methyl-1,3-thiazol- 5-yl)phenyl]methyl}pyrrolidine-2-carboxamide From Intermediate F22. LCMS (Method A2): 3.04 min; m/z: 556.3 [M + 2H]²⁺. ¹H NMR (400 MHz, DMSO-d₆): 8.97 (s, 1H), 8.56 (t, J = 6.1 Hz, 1H), 8.20 (d, J = 2.4 Hz, 1H), 8.14 (s, 1H), 7.88 (d, J = 9.4 Hz, 1H), 7.80 (dd, J = 8.5, 2.5 Hz, 1H), 7.59-7.55 (m, 2H), 7.54 (s, 1H), 7.40 (q, J = 8.2 Hz, 5H), 7.16 (t, J = 8.8 Hz, 2H), 7.11 (d, J = 8.1 Hz, 1H), 7.08 (s, 1H), 6.87 (d, J = 8.5 Hz, 1H), 5.76 (s, 2H), 5.65 (q, J = 6.4 Hz, 1H), 5.13 (s, 1H), 4.55 (d, J = 9.3 Hz, 1H), 4.48-4.39 (m, 2H), 4.35 (s, 1H), 4.29 (t, J = 6.6 Hz, 2H), 4.21 (dd, J = 15.9, 5.5 Hz, 1H), 3.66 (s, 2H), 3.58 (s, 3H), 2.44 (s, 3H), 2.23 (ddt, J = 58.0, 14.1, 7.2 Hz, 3H), 2.07- 1.87 (m, 3H), 1.75 (p, J = 7.3 Hz, 2H), 1.57 (d, J = 6.4 Hz, 6H), 1.41 (q, J = 7.5 Hz, 2H). |
| 1066 | 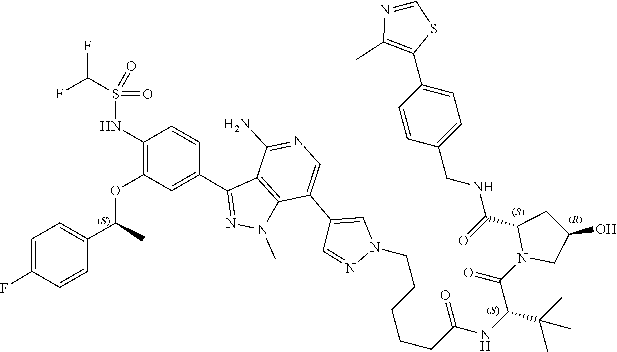 | (2S,4R)-1-[(2S)-2-[6-(4-{4-amino-3-[4-(difluoromethanesulfonamido)-3-[(1S)- 1-(4-fluorophenyl)ethoxy]phenyl]-1-methyl-1H-pyrazolo[4,3-c]pyridin-7-yl}- 1H-pyrazol-1-yl)hexanamido]-3,3-dimethylbutanoyl]-4-hydroxy-N-{[4-(4- methyl-1,3-thiazol-5-yl)phenyl]methyl}pyrrolidine-2-carboxamide From Intermediate F4. LCMS (Method H): 1.99 min, m/z: 1085.2 [M + H]⁺. ¹H NMR (400 MHz, DMSO-d₆): 8.99 (s, 1H), 8.57 (t, J = 6.0 Hz, 1H), 8.15 (s, 1H), 7.95-7.90 (m, 1H), 7.88 (d, J = 9.0 Hz, 1H), 7.63-7.51 (m, 4H), 7.47-7.34 (m, 5H), 7.23-7.12 (m, 3H), 7.12 (q, J = 1.8 Hz, 1H), 7.00 (t, J = 52.1 Hz, 1H), 5.73 (s, 1H), 5.66 (q, J = 6.2 Hz, 1H), 4.54 (d, J = 9.3 Hz, 1H), 4.51-4.39 (m, 2H), 4.36 (s, 1H), 4.29-4.10 (m, 4H), 3.69 (d, J = 17.2 Hz, 5H), 2.45 (s, 3H), 2.28 (dt, J = 14.6, 7.6 Hz, 1H), 2.15 (q, J = 7.0 Hz, 1H), 2.04 (t, J = 10.8 Hz, 1H), 1.91 (td, J = 8.4, 4.1 Hz, 1H), 1.83 (t, J = 7.6 Hz, 2H), 1.59 (d, J = 6.3 Hz, 3H), 1.56-1.48 (m, 1H), 1.24 (t, J = 7.7 Hz, 2H), 0.93 (s, 9H). |
| 1067 | 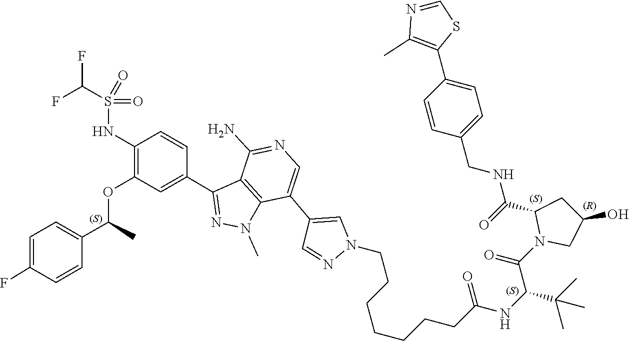 | (2S,4R)-1-[(2S)-2-[8-(4-{4-amino-3-[4-(difluoromethanesulfonamido)-3-[(1S)- 1-(4-fluorophenyl)ethoxy]phenyl]-1-methyl-1H-pyrazolo[4,3-c]pyridin-7-yl}- 1H-pyrazol-1-yl)octanamido]-3,3-dimethylbutanoyl]-4-hydroxy-N-{[4-(4- methyl-1,3-thiazol-5-yl)phenyl]methyl}pyrrolidine-2-carboxamide From Intermediate F5. LCMS (Method H): 2.12 min, m/z: 1112.2 [M + H]⁺. ¹H NMR (400 MHz, DMSO-d₆): 8.99 (s, 1H), 8.57 (t, J = 6.1 Hz, 1H), 8.15 (s, 1H), 7.93 (s, 1H), 7.85 (d, J = 9.4 Hz, 1H), 7.63-7.51 (m, 5H), 7.47-7.34 (m, 6H), 7.17 (td, J = 8.5, 1.9 Hz, 4H), 7.11 (d, J = 2.4 Hz, 2H), 7.00 (t, J = 53.1 Hz, 2H), 5.71 (s, 2H), 5.66 (d, J = 6.4 Hz, 1H), 4.55 (d, J = 9.4 Hz, 1H), 4.50-4.39 (m, 3H), 4.35 (s, 1H), 4.28-4.01 (m, 7H), 3.70 (s, 3H), 3.67 (d, J = 10.1 Hz, 3H), 2.45 (s, 4H), 2.33-2.20 (m, 2H), 2.19-1.87 (m, 5H), 1.83 (d, J = 8.1 Hz, 2H), 1.58 (d, J = 6.2 Hz, 4H), 1.47 (t, J = 7.4 Hz, 3H), 1.30-1.22 (m, 5H), 0.93 (s, 9H). |

### Compound 1019: (2S,4R)-1-[(2S)-2-{2-[2-(2-{4-[4-amino-3-(4-ethanesulfonamidophenyl)-1-methyl-1H-pyrazolo[4,3-c]pyridin-7-yl]-1H-pyrazol-1-yl}ethoxy)ethoxy]acetamido}-3,3-dimethylbutanoyl]-4-hydroxy-N-{[4-(4-methyl-1,3-thiazol-5-yl)phenyl]methyl}pyrrolidine-2-carboxamide

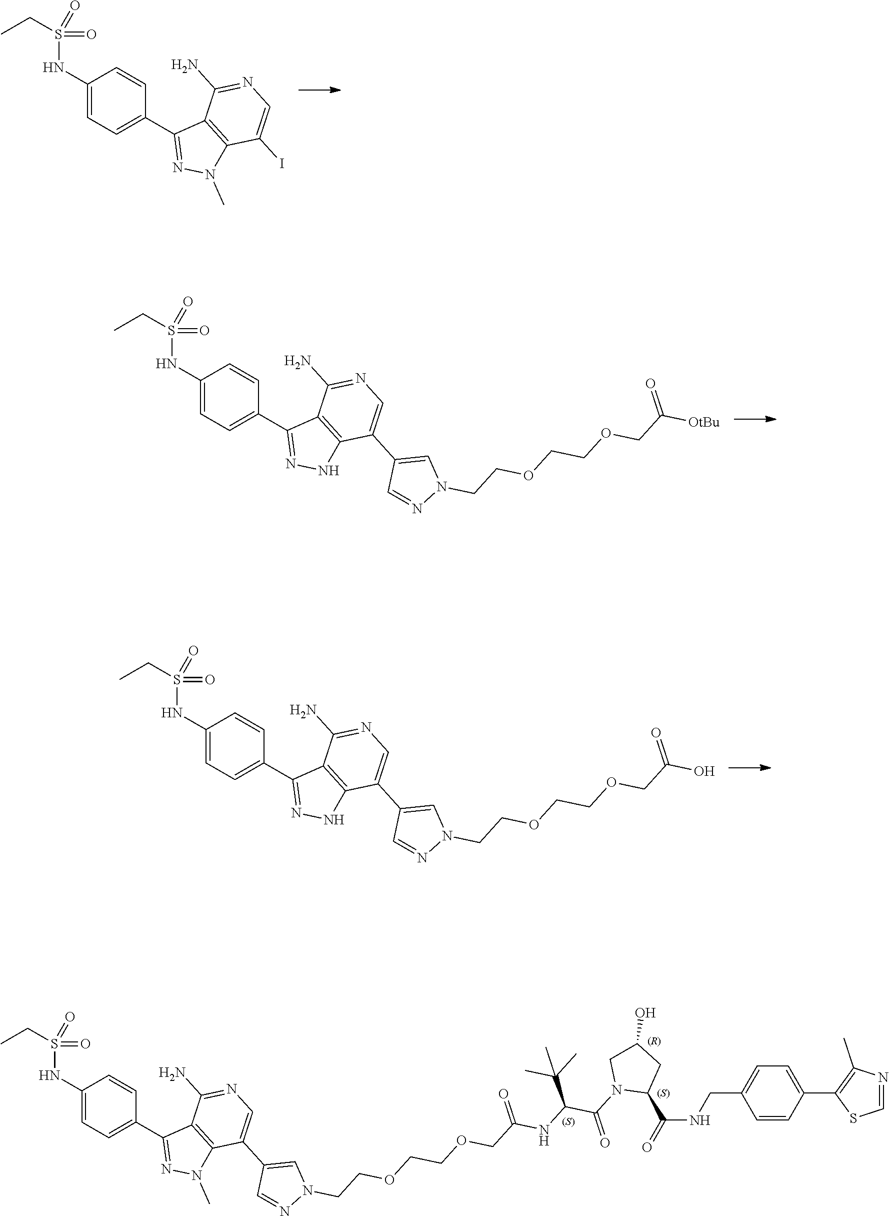

### Step 1: tert-butyl 2-[2-(2-{4-[4-amino-3-(4-ethanesulfonamidophenyl)-1-methyl-1H-pyrazolo [4,3-c]pyridin-7-yl]-1H-pyrazol-1-yl}ethoxy)ethoxy]acetate

To a solution of N-(4-{4-amino-7-iodo-1-methyl-1H-pyrazolo[4,3-c]pyridin-3-yl}phenyl)ethane-1-sulfonamide (500 mg, 1.09 mmol) in 1,4-dioxane (12 mL) and water (4 mL) were added tert-10 butyl 2-(2-{2-[4-(4,4,5,5-tetramethyl-1,3,2-dioxaborolan-2-yl)-1H-pyrazol-1-yl]ethoxy}ethoxy) acetate (645 mg, 1.63 mmol), Na₂CO₃ (346 mg, 3.27 mmol) and Pd(dpp)Cl₂ (79.7 mg, 109 μmol), and the reaction was heated at 80° C. with microwave irradiation for 2 h. The mixture was concentrated, and the residue was purified by column chromatography (DCM/MeOH=20/1) to afford the title product (220 mg, 34%) as a yellow solid. LCMS (method A): 2.88 min, 15 m/z: 600.3 [M+H]⁺.

### Step 2: 2-[2-(2-{4-[4-amino-3-(4-ethanesulfonamidophenyl)-1-methyl-1H-pyrazolo[4,3-c]pyridin-7-yl]-1H-pyrazol-1-yl}ethoxy)ethoxy]acetic acid

A solution of tert-butyl 2-[2-(2-{4-[4-amino-3-(4-ethanesulfonamidophenyl)-1-methyl-1H-pyrazolo[4,3-c]pyridin-7-yl]-1H-pyrazol-1-yl}ethoxy)ethoxy]acetate (220 mg, 0.37 mmol) in MeOH (9 mL) and 37% aq. NaOH (3 mL) was stirred at 80° C. for 1 h. The mixture was concentrated and the residue was purified by column chromatography to afford the title compound (60 ma. 30%) as a white solid. LCMS (method A): 2.43 min, m/z: 544.2 [M+H]⁺.

### Step 3: (2S,4R)-1-[(2S)-2-{2-[2-(2-{4-[4-amino-3-(4-ethanesulfonamidophenyl)-1-methyl-1H-pyrazolo[4,3-c]pyridin-7-yl]-1H-pyrazol-1-yl}ethoxy)ethoxy]acetamido}-3,3-dimethylbutanoyl]-4-hydroxy-N-{[4-(4-methyl-1,3-thiazol-5-yl)phenyl]methyl}pyrrolidine-2-carboxamide

To a solution of 2-[2-(2-{4-[4-amino-3-(4-ethanesulfonamidophenyl)-1-methyl-1H-pyrazolo [4,3-c]pyridin-7-yl]-1H-pyrazol-1-yl}ethoxy)ethoxy]acetic acid (30 mg, 55.2 μmol) in DMF (2 mL) were added HATU (41.8 mg, 110 μmol) and Et₃N (16.6 mg, 165 μmol). The reaction mixture was stirred at RT for 30 min, then (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide (23.7 mg, 55.1 μmol) was added. After stirring at RT overnight, the mixture was concentrated and the residue was purified by Prep-TLC (DCM/MeOH=10/1) to afford the title product (40 mg, 76%) as a yellow solid. LCMS (method A): 2.66 min, m/z: 478.7 [M+H]⁺. ¹H NMR (400 MHz, DMSO-d₆): 10.19 (s, 1H), 10.13 (s, 1H), 8.97 (s, 1H), 8.63 (t, J=4.8 Hz, 1H), 8.00 (s, 1H), 7.64 (s, 1H), 7.57 (d, J=6.0 Hz, 3H), 7.46 (d, J=9.6 Hz, 1H), 7.39 (s, 1H), 7.37 (s, 4H), 6.75 (s, 1H), 5.20 (s, 1H), 4.57 (d, J=9.6 Hz, 1H), 4.44 (q, J=4.0 Hz, 1H), 4.40-4.35 (m, 3H), 4.25-4.20 (m, 1H), 3.95 (s, 2H), 3.85 (q, J=4.8 Hz, 2H), 3.73 (s, 3H), 3.67-3.58 (m, 6H), 3.19 (q, J=7.2 Hz, 2H), 2.42 (s, 2H), 2.08-2.03 (m, 1H), 1.92-1.85 (m, 1H), 1.23 (s, 3H), 0.91 (s, 9H).

The following compounds were prepared according to the procedure for the synthesis of Compound 1019 using the indicated Intermediate AB in Step 1.

TABLE 12
Compounds 1021 and 1029

| Example | | |
|---|---|---|
| No | Structure | Compound Name and Analytical Data |
| 1021 | 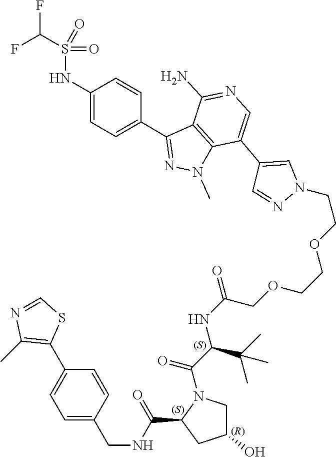 | (2S,4R)-1-[(2S)-2-[7-(4-{4-amino-3-[4-(difluoromethanesulfonamido) phenyl]-1-methyl-1H-pyrazolo[4,3-c]pyridin-7-yl}-1H-pyrazol-1- yl)heptanamido]-3,3-dimethylbutanoyl]-4-hydroxy-N-{[4-(4-methyl- 1,3-thiazol-5-yl)phenyl]methyl}pyrrolidine-2-carboxamide From Intermediate A1B18. LCMS (method A): 2.76 min, m/z: 489.6 [M + 2H]²⁺. ¹H NMR (400 MHz, DMSO-d₆): 8.97 (s, 1H), 8.66 (s, 1H), 8.00 (s, 1H), 7.64- 7.58 (m, 5H), 7.47-7.37 (m, 8H), 7.30-7.04 (m, 1H), 6.77 (s, 1H), 5.24 (s, 1H), 4.55 (d, J = 9.2 Hz, 1H),4.46-4.24 (m, 8H), 3.95 (s, 2H), 3.85 (s, 3H), 3.73 (s, 3H), 3.58 (s, 5H), 2.42 (s, 3H), 0.91 (s, 9H). |
| 1029 | 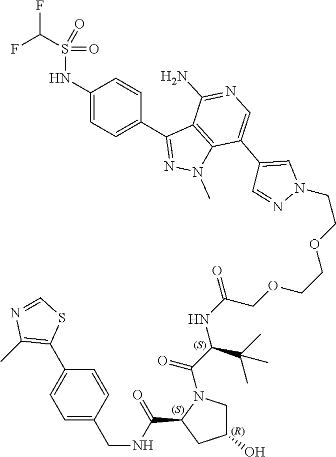 | (2S,4R)-1-[(2S)-2-{2-[2-(2-{4-[3-(4-ethanesulfonamido-3-fluorophenyl)- 1,4-dimethyl-1H-pyrazolo[4,3-c]pyridin-7-yl]-1H-pyrazol-1-yl}ethoxy) ethoxy]acetamido}-3,3-dimethylbutanoyl]-4-hydroxy-N-{[4-(4-methyl- 1,3-thiazol-5-yl)phenyl]methyl}pyrrolidine-2-carboxamide From Intermediate A1B20. LCMS (method A): 2.65 min, m/z: 487.7 [M + 2H]²⁺. ¹H NMR (400 MHz, DMSO-d₆): 9.83 (s, 1H), 8.96 (s, 1H), 8.59 (t, J = 6.1 Hz, 1H), 7.92 (s, 1H), 7.61-7.52 (m, 3H), 7.50-7.44 (m, J = 17.3, 9.9 Hz, 3H), 7.37 (s, 4H), 5.91 (s, 2H), 5.75 (s, 1H), 5.16 (s, 1H), 4.56 (d, J = 9.6 Hz, 1H), 4.43-4.35 (m, J = 33.6 Hz, 5H), 4.25-4.20 (m, J = 15.8, 5.4 Hz, 2H), 4.10 (d, J = 5.4 Hz, 1H), 3.95 (s, 2H), 3.86 (d, J = 6.0 Hz, 2H), 3.72 (s, 3H), 3.64-3.58(m, J = 7.0 Hz, 6H), 3.18 (q, J= 6.3, 5.3 Hz, 4H), 2.42 (s, 3H), 2.08-1.85 (m, 4H), 1.29 (t, J = 7.4 Hz, 3H), 0.91 (s, 9H). |

### Compound 1020: (2S,4R)-1-[(2S)-2-(7-{4-[4-amino-3-(4-ethanesulfonamidophenyl)-1-methyl-1H-pyrazolo[4,3-c]pyridin-7-yl]-1H-pyrazol-1-yl}heptanamido)-3,3-dimethylbutanoyl]-4-hydroxy-N-{[4-(4-methyl-1,3-thiazol-5-yl)phenyl]methyl}pyrrolidine-2-carboxamide

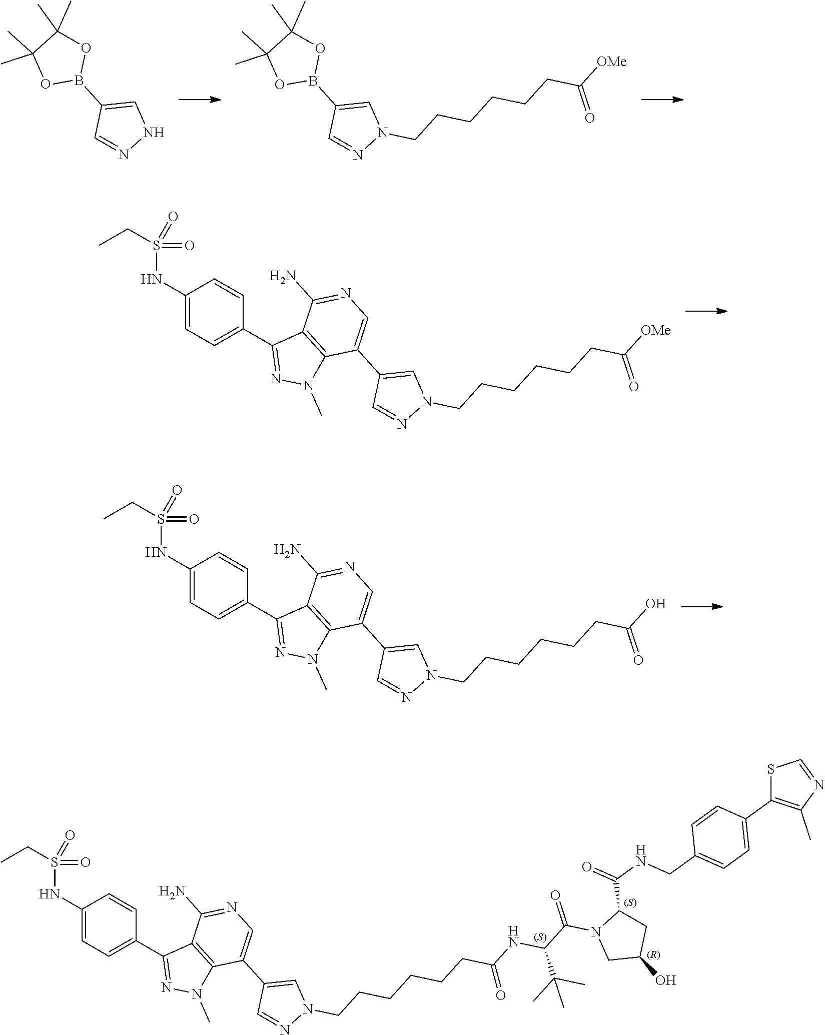

### Step 1: tert-butyl 7-[4-(4,4,5,5-tetramethyl-1,3,2-dioxaborolan-2-yl)-1H-pyrazol-1-yl]heptanoate

To a solution of methyl 7-bromoheptanoate (5 g, 22.4 mmol) in DMF (15 mL) were added 4-(4,4,5,5-tetramethyl-1,3,2-dioxaborolan-2-yl)-1H-pyrazole (4.34 g, 22.4 mmol) and Cs₂CO₃ (14.5 g, 44.8 mmol), and the reaction was heated at 90° C. for 1.5 h. Once cooled, the mixture was concentrated and the residue was diluted with water (50 mL). The aqueous mixture was extracted with EtOAc (50 mL×3), and the combined organics were washed with water and brine, dried (Na₂SO₄) and concentrated. The residue was purified by column (PE/EA=5/1) to afford the title compound (6 g, 71%) as a yellow oil. LCMS (Method A): 4.03 min, m/z: 337.3 [M+H]⁺.

### Step 2: methyl 7-{4-[4-amino-3-(4-ethanesulfonamidophenyl)-1-methyl-1H-pyrazolo[4,3-c]pyridin-7-yl]-1H-pyrazol-1-yl}heptanoate

To a solution of N-(4-{4-amino-7-iodo-1-methyl-1H-pyrazolo[4,3-c]pyridin-3-yl}phenyl)ethane-1-sulfonamide (500 mg, 1.09 mmol) in 1,4-dioxane (12 mL) and H₂O (4 mL) were added methyl 7-[4-(4,4,5,5-tetramethyl-1,3,2-dioxaborolan-2-yl)-1H-pyrazol-1-yl]heptanoate (548 mg, 1.63 mmol), Na₂CO₃ (346 mg, 3.27 mmol) and Pd(dppf)Cl₂ (79.7 mg, 109 μmol). After stirring at 80° C. for 2 h under microwave, the mixture was concentrated and the residue was purified by Prep-TLC (DCM/MeOH=20/1, v/v) to afford the title compound (330 mg, 56%) as a yellow solid. LCMS (Method A): 2.93 min. m/z: 540.3 [M+H]⁺.

### Step 3: 7-{4-[4-amino-3-(4-ethanesulfonamidophenyl)-1-methyl-1H-pyrazolo[4,3-c]pyridin-7-yl]-1H-pyrazol-1-yl}heptanoic acid

A solution of methyl 7-{4-[4-amino-3-(4-ethanesulfonamidophenyl)-1-methyl-1H-pyrazolo[4,3-c]pyridin-7-yl]-1H-pyrazol-1-yl}heptanoate (300 mg, 0.56 mmol) in MeOH (9 mL) and NaOH (3 mL) was stirred at 80° C. for 1 h. The mixture was concentrated in vacuo, then the mixture was adjusted to pH=3-4 with 2M HCl. The precipitate was collected via filtration, washed with water and dried to afford the title compound (230 mg, 79%) as a yellow solid. LCMS (method A): 2.68 min, m/z: 526.2 [M+H]⁺.

### Step 4: (2S,4R)-1-[(2S)-2-(7-{4-[4-amino-3-(4-ethanesulfonamidophenyl)-1-methyl-1H-pyrazolo[4,3-c]pyridin-7-yl]-1H-pyrazol-1-yl}heptanamido)-3,3-dimethylbutanoyl]-4-hydroxy-N-{[4-(4-methyl-1,3-thiazol-5-yl)phenyl]methyl}pyrrolidine-2-carboxamide

To a solution of 7-{4-[4-amino-3-(4-ethanesulfonamidophenyl)-1-methyl-1H-pyrazolo[4,3-c]pyridin-7-yl]-1H-pyrazol-1-yl}heptanoic acid (150 mg, 0.29 mmol) in DMF (2 mL) were added HATU (216 mg, 570 μmol) and Et₃N (86.5 mg, 855 μmol). The reaction mixture was stirred at RT for 30 min before (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide (122 mg, 285 μmol) was added.The reaction was stirred at RT overnight, then concentrated in vacuo. The residue was purified by Prep-TLC (DCM/MeOH=10/1) to afford the title compound (80 mg, 30%) as a yellow solid. LCMS (method A): 2.92 min, m/z: 469.7 [M+H]²⁺. ¹H NMR (400 MHz, DMSO-d₆): 10.08 (s, 1H), 8.98 (s, 1H), 8.58 (t, J=6.0 Hz, 1H), 7.96 (s, 1H), 7.88 (d, J=9.6 Hz, 1H), 7.61-7.56 (m, 4H), 7.43-7.36 (m, 6H), 6.15 (s, 2H), 5.15 (s, 1H), 4.54 (d, J=9.6 Hz, 1H), 4.43 (q, J=8.0 Hz, 2H), 4.34 (s, 1H), 4.22 (d, J=5.6 Hz, 1H), 4.18-4.13 (m, 3H), 3.74 (s, 3H), 3.65 (s, 2H), 3.19 (q, J=7.2 Hz, 2H), 2.44 (s, 3H), 2.29-2.22 (m, 1H), 2.15-2.06 (m, 1H), 2.06-1.97 (m, 1H), 1.92-1.86 (m, 1H), 1.84-1.78 (m, 2H), 1.53-1.42 (m, 2H), 1.23 (t, J=7.2 Hz, 5H), 0.93 (s, 9H).

### Compound 1022: (2S,4R)-1-[(2S)-2-[7-(4-{4-amino-3-[4-(difluoromethanesulfonamido) phenyl]-1-methyl-1H-pyrazolo[4,3-c]pyridin-7-yl}-1H-pyrazol-1-yl)heptanamido]-3,3-dimethylbutanoyl]-4-hydroxy-N-{[4-(4-methyl-1,3-thiazol-5-yl)phenyl]methyl}pyrrolidine-2-carboxamide

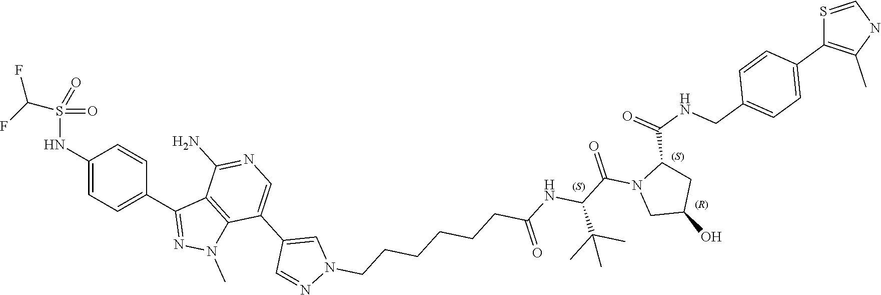

Compound 1022 was obtained as a yellow solid (70 mg, 45%) from Intermediate A1B18 according to the procedure for the synthesis of compound 1020. LCMS (method A): 1.44 min, m/z: 480.9 [M+H]²⁺. ¹H NMR (400 MHz, DMSO-d₆): 8.98 (s, 1H), 8.61 (t, J=5.6 Hz, 1H), 7.99 (s, 1H), 7.88 (d, J=9.2 Hz, 1H), 7.63 (s, 1H), 7.59 (d, J=6.4 Hz, 3H), 7.39 (q, J=8.0 Hz, 6H), 7.16 (t, J=52.6 Hz, 1H), 6.56 (s, 2H), 5.18 (s, 1H), 4.54 (d, J=9.6 Hz, 1H), 4.44-4.40 (m, 2H), 4.34 (s, 1H), 4.23-4.14 (m, 3H), 3.75 (s, 3H), 3.65 (s, 2H), 2.44 (s, 3H), 2.27-2.08 (m, 2H), 2.06-1.86 (m, 2H), 1.89-1.79 (m, 2H), 1.50-1.45 (s, 2H), 0.93 (s, 9H).

### Compound 1037: (2S,4R)-1-[(2S)-2-({4-[2-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)ethoxy]phenyl}formamido)-3,3-dimethylbutanoyl]-4-hydroxy-N-{[4-(4-methyl-1,3-thiazol-5-yl)phenyl]methyl}pyrrolidine-2-carboxamide

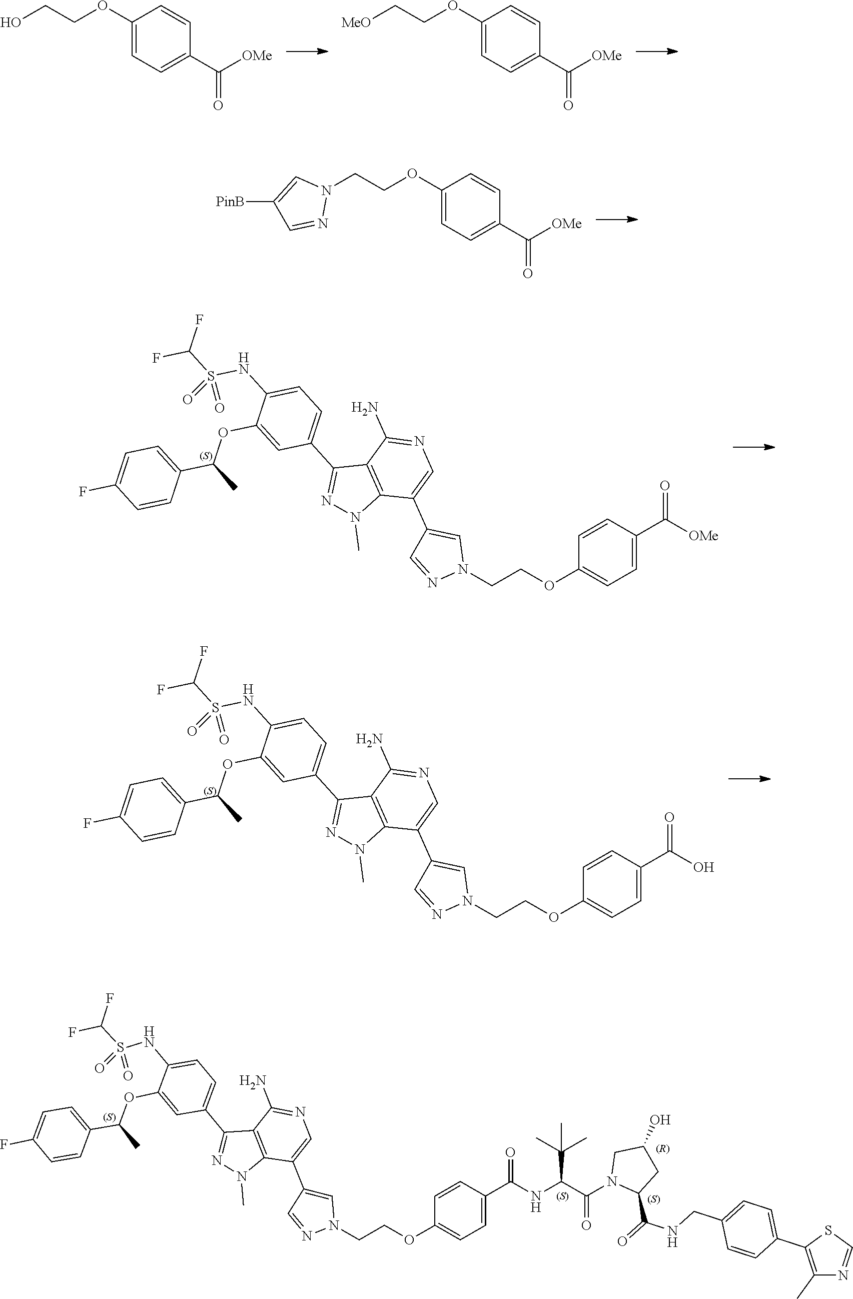

### Step 1: methyl 4-[2-(methanesulfonyloxy)ethoxy]benzoate

A mixture of methyl 4-(2-hydroxyethoxy)benzoate (2 g, 10.1 mmol), Et₃N (4.08 g, 40.4 mmol) and MsCl (2.31 g, 20.2 mmol) in DCM (30 mL) was stirred at RT overnight. The mixture was concentrated, and the residue was purified by column chromatography to afford the title product (2.70 g, 97%) as a yellow oil. LCMS (method A): 3.17 min, m/z 275.0 [M+H]⁺.

### Step 2: methyl 4-{2-[4-(4,4,5,5-tetramethyl-1,3,2-dioxaborolan-2-yl)-1H-pyrazol-1-yl]ethoxy}benzoate

A mixture of 4-(4,4,5,5-tetramethyl-1,3,2-dioxaborolan-2-yl)-1H-pyrazole (500 mg, 2.57 mmol), methyl 4-[2-(methanesulfonyloxy)ethoxy]benzoate (1.40 g, 5.14 mmol) and Cs₂CO₃ (3.32 g, 10.2 mmol) in MeCN (30 mL) was stirred at 80° C. overnight. Once cooled, the mixture was filtered and concentrated. The residue was purified by column chromatography to afford the title product (800 mg, 84%) as a yellow oil. LCMS (method A): 3.76 min, m/z 373.1 [M+H]⁺

### Step 3: methyl 4-[2-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)ethoxy]benzoate

A mixture of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (Intermediate AB4, 623 mg, 1.01 mmol), methyl 4-{2-[4-(4,4,5,5-tetramethyl-1,3,2-dioxaborolan-2-yl)-1H-pyrazol-1-yl]ethoxy}benzoate (720 mg, 1.27 mmol), Na₂CO₃ (269 mg, 2.54 mmol), and Pd (dppf)Cl₂—CH₂Cl₂ (103 mg, 127 μmol) 80% aq. Dioxane (12 mL) was stirred at 100° C. overnight. The mixture was concentrated and the residue was purified by Prep-TLC (DCM:MeOH, 20:1) to afford the title product (250 mg, 27%) as a yellow oil. LCMS (method A): 3.44 min, m/z 736.1 [M+H]⁺.

### Step 4: 4-[2-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)ethoxy]benzoic acid

A solution of methyl 4-[2-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)ethoxy]benzoate (200 mg, 271 μmol) and NaOH (43.1 mg, 1.08 mmol) in 66% aq. MeOH (6 mL) was stirred at 60° C. under N₂ overnight. The mixture was poured into water (20 mL) and washed with EtOAc (2×20 mL). The aqueous layer was adjust to pH=1 and extracted with EtOAc (3×20 mL). The combined organic extracts were dried over Na₂SO₄ and concentrated to afford the title product (16.0 mg, 8%) as a yellow oil. LCMS (method A): 3.47 min, m/z 721.8 [M+H]⁺.

### Step 5: (2S,4R)-1-[(2S)-2-({4-[2-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl-}1H-pyrazol-1-yl)ethoxy]phenyl}formamido)-3,3-dimethylbutanoyl]-4-hydroxy-N-{[4-(4-methyl-1,3-thiazol-5-yl)phenyl]methyl}pyrrolidine-2-carboxamide

A mixture of 4-[2-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)ethoxy]benzoic acid (20 mg, 27.7 μmol) and (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide hydrochloride (13.5 mg, 29.0 μmol) Et₃N (11.1 mg, 110 μmol) and HATU (11.5 mg, 30.4 μmol) in MeCN (3 mL) was stirred at RT overnight. The mixture was poured into water and extracted with ethyl acetate (2×30 mL). The combined organic layers was washed with brine (2×50 mL) and concentrated. The residue was purified by prep-TLC to afford the title product (10.0 mg, 32%) as a yellow solid. LCMS (method A): 3.21 min, m/z 1134.1 [M+H]⁺. ¹H NMR (400 MHz, CDCl₃): 8.66 (s, 1H), 7.68-7.61 (m, 3H), 7.55 (d, J=3.4 Hz, 2H), 7.48 (s, 1H), 7.39-7.27 (m, 7H), 7.18 (dd, J=8.2, 1.8 Hz, 1H), 7.04 (d, J=1.8 Hz, 1H), 6.98 (t, J=8.4 Hz, 2H), 6.81 (d, J=8.4 Hz, 2H), 6.73 (d, J=8.8 Hz, 1H), 6.34 (t, J=53.7 Hz, 1H), 5.43 (q, J=6.5 Hz, 1H), 5.01 (s, 1H), 4.71 (t, J=9.0 Hz, 2H), 4.54 (dd, J=14.9, 5.6 Hz, 4H), 4.43 (t, J=4.9 Hz, 2H), 4.32 (dd, J=14.9, 5.2 Hz, 1H), 4.13 (d, J=11.4 Hz, 1H), 3.65 (s, 3H), 3.47 (s, 2H), 2.49 (s, 4H), 1.64 (d, J=6.3 Hz, 3H), 0.99 (s, 9H).

### Compound 1039: (2S,4R)-1-[(2S)-2-({3-[2-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)ethoxy]phenyl}formamido)-3,3-dimethylbutanoyl]-4-hydroxy-N-{[4-(4-methyl-1,3-thiazol-5-yl)phenyl]methyl}pyrrolidine-2-carboxamide

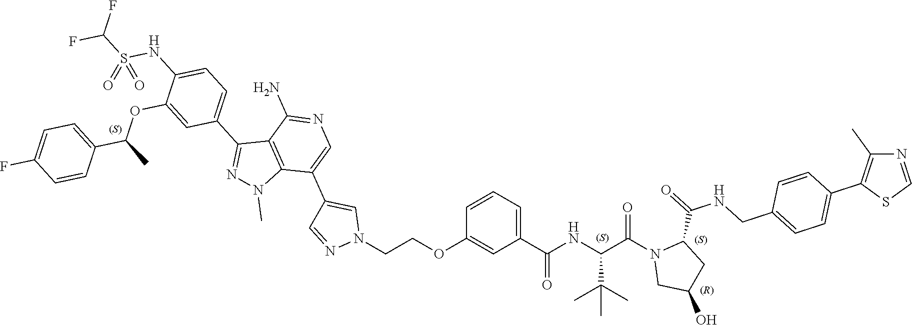

The title product was prepared from methyl 3-[2-(methanesulfonyloxy)ethoxy]benzoate according to the procedures for the synthesis of Compound 1037. The compound was obtained as a yellow solid (21.0 mg, 17%). LCMS (method A): 2.93 min, m/z 1134.1 [M+H]⁺. ¹H NMR (400 MHz, CDCl₃): 8.98 (s, 1H), 8.59 (t, J=6 Hz, 1H), 8.06 (s, 2H), 7.66 (s, 1H), 7.59-7.55 (m, 3H), 7.48-7.38 (m, 8H), 7.19-7.07 (m, 5H), 7.04 (t, J=52.8 Hz, 1H), 6.11 (s, 2H), 5.65 (q, J=6.4 Hz, 1H), 5.17 (s, 1H), 4.78 (d, J=9.2 Hz, 1H), 4.61-4.21 (m, 9H), 3.72 (d, J=2.8 Hz, 2H), 3.67 (s, 3H), 2.44 (s, 3H), 1.59 (d, J=6 Hz, 3H), 1.03 (s, 9H).

### Synthesis of Intermediates G

### Intermediate G1: (2S,4R)-1-[(2S)-3,3-dimethyl-2-(pent-4-ynamido)butanoyl]-4-hydroxy-N-{[4-(4-methyl-1,3-thiazol-5-yl)phenyl]methyl}pyrrolidine-2-carboxamide

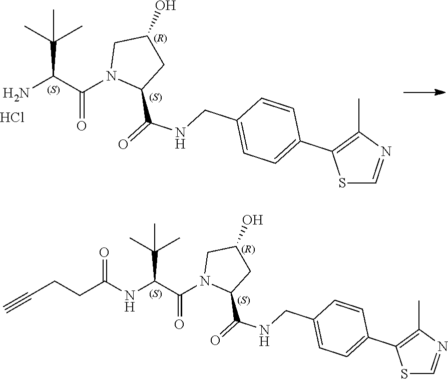

### Step 1: (2S,4R)-1-[(2S)-3,3-dimethyl-2-(pent-4-ynamido)butanoyl]-4-hydroxy-N-{[4-(4-methyl-1,3-thiazol-5-yl)phenyl]methyl}pyrrolidine-2-carboxamide

A mixture of 4-pentynoic acid (45.5 mg, 464 μmol), Et₃N (187 mg, 1.85 mmol) and HATU (353 mg, 929 μmol) in DMF (5 mL) was stirred at RT for 30 mins before (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide (200 mg, 465 μmol) was added. The reaction was stirred at RT overnight, then poured into water (20 mL) and extracted with EtOAc (30 mL×3). The combined organic phases were dried (Na₂SO₄) and concentrated, and the residue was purified by prep-TLC (DCM/MeOH=10/1, v/v) to afford the title product (190 mg, 80%) as a yellow solid.

The following intermediates were similarly prepared from the appropriate acid according to the procedure for the synthesis of Intermediate G1.

TABLE 13
Intermediates G2 to G4

| Intermediate | Name | Structure | LCMS data | SM |
|---|---|---|---|---|
| G2 | (2S,4R)-1-[(2S)-2-(hept-6- ynamido)-3,3-dimethyl- butanoyl]-4-hydroxy-N-{[4- (4- methyl-1,3-thiazol-5-yl) phenyl]methyl}pyrrolidine- 2-carboxamide | 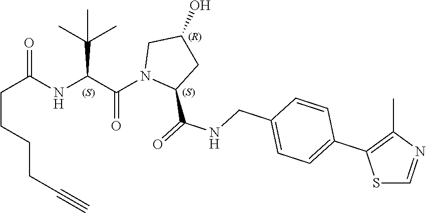 | (method A): 3.17 min, 561.2 [M + Na]+. | 6- Heptynoic acid |
| G3 | (2S,4R)-1-[(2S)-3,3-dimethyl- 2-(non-8-ynamido)butanoyl]- 4-hydroxy-N-{[4-(4-methyl- 1,3-thiazol-5-yl)phenyl] methyl}pyrrolidine-2- carboxamide | 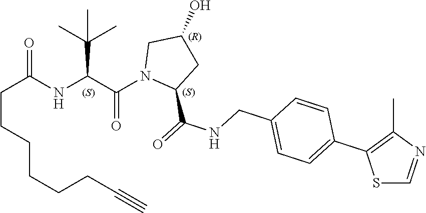 | (method A): 3.46 min, 590.2 [M + Na]+. | 8- Nonynoic acid |
| G4 | (2S,4R)-1-((S)-3,3-dimethyl- 2-(undec-10-ynamido) butanoyl)-4-hydroxy-N- (4-(4-methyl-thiazol-5-yl) benzyl)pyrrolidine- 2-carboxamide | 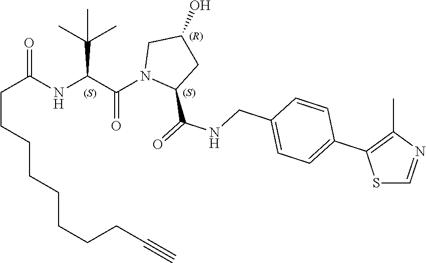 | | 10- Undecynoic acid |

### Intermediate G5: (2S,4R)-1-[(2S)-2-[2-(4-ethynylpiperidin-1-yl)acetamido]-3,3-dimethylbutanoyl]-4-hydroxy-N-{[4-(4-methyl-1,3-thiazol-5-yl)phenyl]methyl}pyrrolidine-2-carboxamide

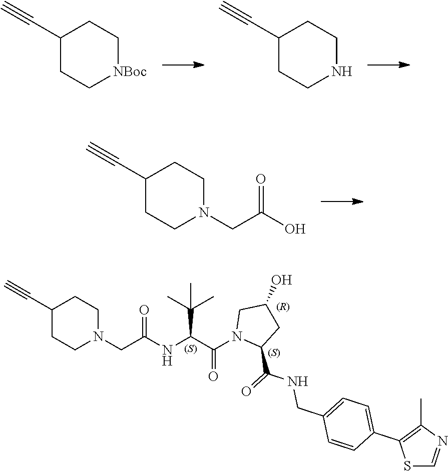

### Step 1: 4-ethynylpiperidine

To a mixture of tert-butyl 4-ethynylpiperidine-1-carboxylate (2.2 g, 10.5 mmol) in dioxane (2 mL) was added 4M HCl in dioxane (2 mL) and the reaction was stirred at RT for 2 h. The mixture was adjusted to pH=8 with aq. Na₂CO₃ solution, then diluted with water (30 mL) and extracted with EtOAc (30 mL×3). The combined organic phases were dried over Na₂SO₄ and concentrated to afford the title product (1.0 g, 88%) as a white solid. LCMS (method A): 0.81 min, m/z: 110.1 [M+H]⁺.

### Step 2: 2-(4-ethynylpiperidin-1-yl)acetic acid

To a solution of 4-ethynylpiperidine (100 mg, 915 μmol) in DMF (3 mL) were added 2-bromoacetic acid (127 mg, 915 μmol) and Cs₂CO₃ (592 mg, 1.82 mmol), and the reaction was stirred at 80° C. overnight. The mixture was concentrated, and the residue was washed with THE to afford the title product (100 mg, 66%) as a yellow oil. LCMS (method A): 0.99 min, m/z: 168.1 [M+H]⁺.

### Step 3: (2S,4R)-1-[(2S)-2-[2-(4-ethynylpiperidin-1-yl)acetamido]-3,3-dimethylbutanoyl]-4-hydroxy-N-{[4-(4-methyl-1,3-thiazol-5-yl)phenyl]methyl}pyrrolidine-2-carboxamide

A mixture of 2-(4-ethynylpiperidin-1-yl)acetic acid (50 mg, 299 μmol), HATU (113 mg, 299 μmol), Et₃N (90.7 mg, 897 μmol), and (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide (257 mg, 598 μmol) in DMF (2 mL) was stirred at 100° C. under N₂ overnight. The mixture was concentrated, and the residue was poured into water (10 mL) and extracted with EtOAc (20 mL×3). The combined organic phases were dried over Na₂SO₄ and concentrated. The residue was purified by Prep-TLC (DCM:MeOH=10:1) to afford the title product (50 mg, 29%) as a yellow solid. LCMS (method A): 2.60 min. m/z: 580.2 [M+H]⁺.

### Intermediate G6: (2S,4R)-1-[(2S)-2-[3-(4-ethynylpiperidin-1-yl)propanamido]-3,3-dimethylbutanoyl]-4-hydroxy-N-{[4-(4-methyl-1,3-thiazol-5-yl)phenyl]methyl}pyrrolidine-2-carboxamide

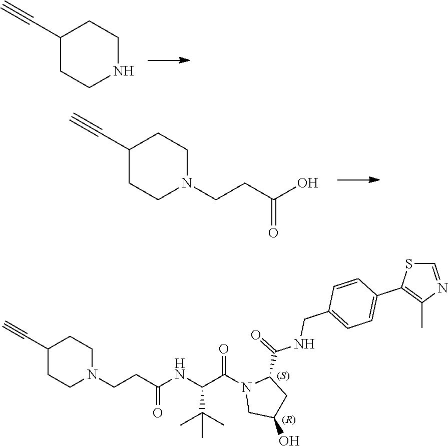

### Step 1: 3-(4-ethynylpiperidin-1-yl)propanoic acid

A mixture of 4-ethynylpiperidine (200 mg, 1.83 mmol), 3-bromopropanoic acid (293 mg, 1.83 mmol) and Cs₂CO₃ (1.87 g, 5.49 mmol) in DMF (3 mL was stirred at 100° C. under N₂ overnight. The mixture was concentrated, and the residue was washed with MeOH to afford the title product (300 mg, 91%) as a white solid. LCMS (Method A): 1.01 min, m/z: 182.0 [M+H]⁺.

### Step 2: (2S,4R)-1-[(2S)-2-[3-(4-ethynylpiperidin-1-yl)propanamido]-3,3-dimethylbutanoyl]-4-hydroxy-N-{[4-(4-methyl-1,3-thiazol-5-yl)phenyl]methyl}pyrrolidine-2-carboxamide

A mixture of 3-(4-ethynylpiperidin-1-yl)propanoic acid (300 mg, 1.65 mmol), (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide (710 mg, 1.65 mmol), Et₃N (499 mg, 4.94 mmol) and HATU (1.25 g, 3.30 mmol) in DMF (5 mL) was stirred at RT overnight. The mixture was concentrated, and the residue was poured into water and extracted with EtOAc. The combined organic phases were dried over Na₂SO₄ and concentrated. The residue was purified by Prep-TLC (DCM/MeOH=20/1) to afford the title product (200 mg, 20%) as a white solid. LCMS (method A): 4.25 min, m/z: 594.0 [M+H]⁺.

### Intermediate G7: (2S,4R)-1-[(2S)-2-[5-(4-ethynylpiperidin-1-yl)pentanamido]-3,3-dimethylbutanoyl]-4-hydroxy-N-{[4-(4-methyl-1,3-thiazol-5-yl)phenyl]methyl}pyrrolidine-2-carboxamide

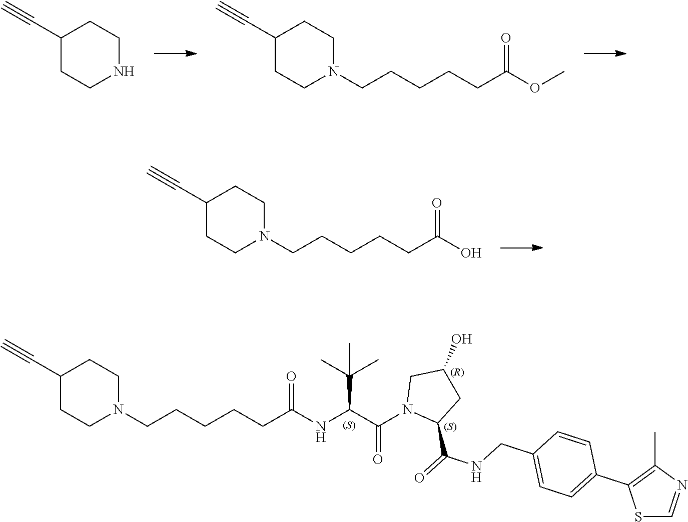

### Step 1: methyl 6-(4-ethynylpiperidin-1-yl)hexanoate

To a stirred solution of 4-ethynylpiperidine hydrochloride (380 mg, 2.60 mmol) in MeCN (25 mL) were added Cs₂CO₃ (2.54 g, 7.80 mmol) and methyl 5-bromopentanoate (760 mg, 3.90 mmol), and the mixture was stirred at 70° C. for 2 h under N₂. Once completed, the mixture was diluted with water and extracted with EA. The organics were dried over Na₂SO₄ and concentrated. The residue was purified by column chromatography (25-50% EtOAc:PE) to afford the title compound (720 mg, >100%).

### Step 2: 6-(4-ethynylpiperidin-1-yl)hexanoic acid

To a solution of methyl 6-(4-ethynylpiperidin-1-yl)hexanoate (620 mg, 2.61 mmol) in MeOH (10 mL) was added 1M NaOH (10 mL), and the mixture was stirred at 60° C. for 2 h. Once completed, the mixture was concentrated and acidified to pH=5-6 with 1 M HCl. The mixture was concentrated to afford the title compound (1 g, >100%).

### Step 3: (2S,4R)-1-[(2S)-2-[5-(4-ethynylpiperidin-1-yl)pentanamido]-3,3-dimethylbutanoyl]-4-hydroxy-N-{[4-(4-methyl-1,3-thiazol-5-yl)phenyl]methyl}pyrrolidine-2-carboxamide

To a solution of (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide hydrochloride (200 mg, 428 μmol) in DMF (6 mL) were added 6-(4-ethynylpiperidin-1-yl)hexanoic acid (191 mg, 856 μmol), DIPEA (165 mg, 1.28 mmol) and HATU (211 mg, 556 μmol), and the mixture was srtirred at RT for 2 h. Once completed, the mixture was diluted with water and extracted with MeOH/DCM=1/10. The organics were washed with brine, dried over Na₂SO₄ and concentrated. The residue was purified by column chromatography (10-30% EtOAc:PE) to afford the title compound (370 mg, >100%) as white solid. LCMS (Method A): 2.88 min, m/z: 622.7 [M+H]⁺.

### Compound 1025: (2S,4R)-1-[(2S)-2-(5-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}pent-4-ynamido)-3,3-dimethylbutanoyl]-4-hydroxy-N-{[4-(4-methyl-1,3-thiazol-5-yl)phenyl]methyl}pyrrolidine-2-carboxamide

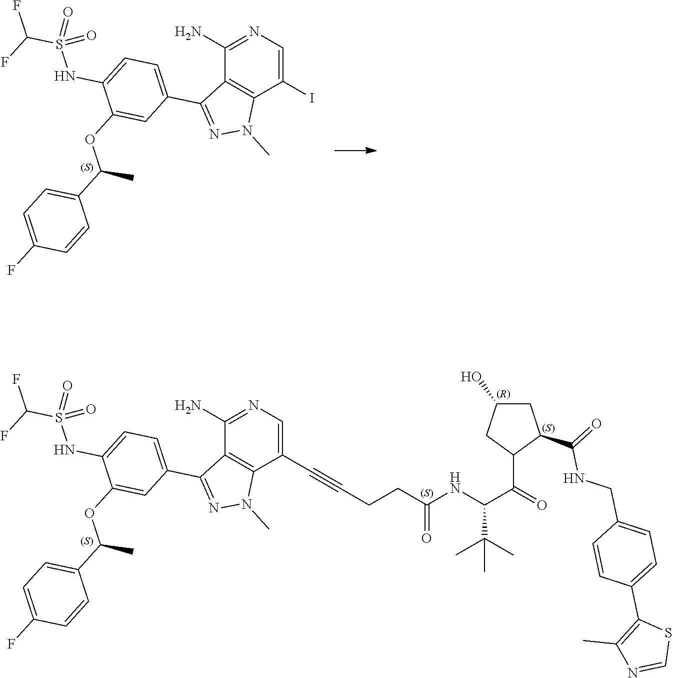

### Step 1: (2S,4R)-1-[(2S)-2-(5-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}pent-4-ynamido)-3,3-dimethylbutanoyl]-4-hydroxy-N-{[4-(4-methyl-1,3-thiazol-5-yl)phenyl]methyl}pyrrolidine-2-carboxamide

A mixture of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (162 mg, 264 μmol), (2S,4R)-1-[(2S)-3,3-dimethyl-2-(pent-4-ynamido)butanoyl]-4-hydroxy-N-{[4-(4-methyl-1,3-thiazol-5-yl)phenyl]methyl}pyrrolidine-2-carboxamide (150 mg, 264 μmol), Cul (50.2 mg, 264 μmol) and Pd(PPh₃)₂Cl₂ (185 mg, 264 μmol) in DMF (5 mL) was heated overnight at 110° C. under N₂. The mixture was concentrated and the residue was purified by column chromatography to afford the title product (40.0 mg, 15%) as a brown solid. LCMS (Method A): 1.26 min, 1000.1 [M+H]⁺. ¹H NMR (400 MHz, DMSO-d₆): 10.59 (s, 1H), 8.97 (d, J=6.2 Hz, 1H), 8.56 (d, J=6.9 Hz, 1H), 8.08 (d, J=9.3 Hz, 1H), 7.80 (s, 1H), 7.57 (dd, J=8.3, 5.5 Hz, 2H), 7.40 (q, J=8.0 Hz, 4H), 7.14 (dt, J=11.1, 7.7 Hz, 4H), 7.01 (s, 1H), 6.89 (d, J=5.8 Hz, 1H), 5.97 (d, J=14.5 Hz, 2H), 5.66 (d, J=6.4 Hz, 1H), 4.59 (d, J=9.3 Hz, 1H), 4.48-4.30 (m, 3H), 4.20 (s, 3H), 4.09 (s, 1H), 3.75-3.59 (m, 2H), 3.17 (d, J=3.2 Hz, 2H), 2.93 (s, 2H), 2.76-2.59 (m, 2H), 2.44 (s, 3H), 2.03 (t, J=10.3 Hz, 1H), 1.91 (s, 1H), 1.57 (d, J=6.3 Hz, 2H), 1.23 (d, J=3.1 Hz, 3H), 1.16 (t, J=7.3 Hz, 3H), 0.93 (s, 5H).

The following examples were similarly prepared from Intermediate A1B1 and the appropriate Intermediate G according to the procedure for the synthesis of Compound 1025.

TABLE 14
Compounds 1026-1028, 1023, 1024 and 1063

| Example | | |
|---|---|---|
| No | Structure | Compound Name and Analytical Data |
| 1026 | 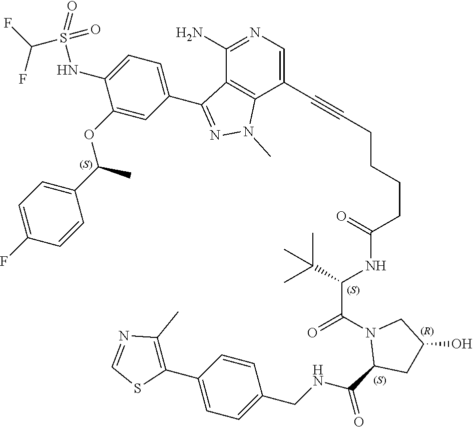 | (2S,4R)-1-[(2S)-2-(7-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}hept-6-ynamido)-3,3- dimethylbutanoyl]-4-hydroxy-N-{[4-(4-methyl-1,3-thiazol-5-yl)phenyl]methyl}pyrrolidine-2-carboxamide From Intermediate G2. LCMS (method A): 3.21 min, m/z: 1028.2 [M + H]⁺. ¹H NMR (400 MHz, MeOD-d₄): 8.88 (s, 1H), 7.76 (s, 1H), 7.57-7.36 (m, 8H), 7.15 (d, J = 8.2 Hz, 1H), 7.11-7.04 (m, 3H), 6.72 (t, J = 53.3 Hz, 1H), 5.57 (q, J = 6.6 Hz, 1H), 4.29 (s, 3H), 3.96 (d, J = 11.1 Hz, 1H), 3.84 (dd, J = 11.0, 3.9 Hz, 1H), 2.55 (t, J = 6.9 Hz, 2H), 2.48 (s, 3H), 2.38 (dd, J = 10.4, 6.3 Hz, 2H), 2.29-2.00 (m, 3H), 1.82 (q, J = 7.6 Hz, 2H), 1.71 (d, J = 6.2 Hz, 3H), 1.68 (d, J = 6.7 Hz, 2H), 1.44-1.26 (m, 5H), 1.06 (s, 9H). |
| 1027 | 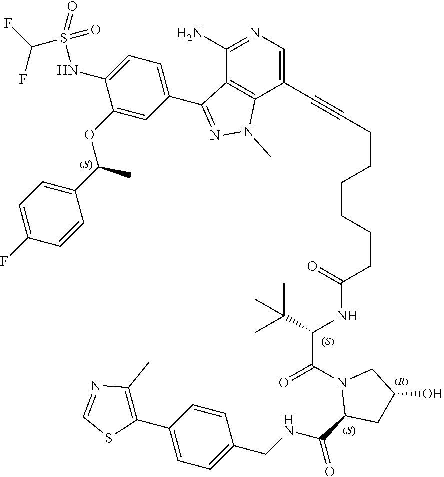 | (2S,4R)-1-[(2S)-2-(9-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}non-8-ynamido)-3,3- dimethylbutanoyl]-4-hydroxy-N-{[4-(4-methyl-1,3-thiazol-5-yl)phenyl]methyl}pyrrolidine-2-carboxamide From Intermediate G3. LCMS (method A): 3.32 min, m/z: 1056.1 [M + H]⁺. ¹H NMR (400 MHz, MeOD-d₄): 8.85 (s, 1H), 7.74 (s, 1H), 7.54-7.35 (m, 7H), 7.13 (d, J = 8.2 Hz, 1H), 7.09-7.02 (m, 3H), 6.69 (t, J = 53.3 Hz, 1H), 5.55 (q, J = 6.5 Hz, 1H), 4.27 (s, 3H), 3.91 (d, J = 11.1 Hz, 1H), 3.80 (dd, J = 11.1, 3.8 Hz, 1H), 2.50 (t, J = 6.9 Hz, 2H), 2.45 (s, 3H), 2.35-2.00 (m, 5H), 1.68 (d, J = 6.5 Hz, 3H), 1.65-1.13 (m, 13H), 1.02 (s, 9H). |
| 1028 | 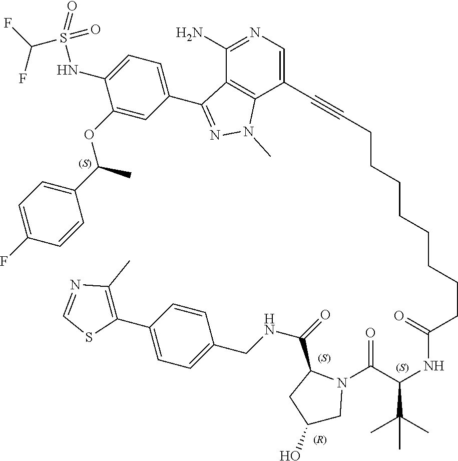 | (2S,4R)-1-[(2S)-2-(11-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}undec-10-ynamido)-3,3- dimethylbutanoyl]-4-hydroxy-N-{[4-(4-methyl-1,3-thiazol-5-yl)phenyl]methyl}pyrrolidine-2-carboxamide From Intermediate G4. LCMS (Method A): 2.91 min, m/z: 542.7 [M + 2H]²⁺. ¹H NMR (400 MHz, DMSO-d₆): 8.98 (d, J = 2.6 Hz, 1H), 8.57 (t, J = 6.2 Hz, 1H), 7.91-7.76 (m, 2H), 7.57 (dd, J = 8.4, 5.4 Hz,2H), 7.40 (q, J = 8.1 Hz, 5H), 7.15 (dd, J = 17.2, 8.0 Hz, 3H), 6.03 (s, 1H), 5.65 (d, J = 6.6 Hz, 1H), 5.13 (d, J = 3.4 Hz, 1H), 4.54 (d, J = 9.3 Hz, 1H), 4.42 (t, J = 8.1 Hz, 2H), 4.35 (s, 1H), 4.21 (d, J = 9.3 Hz, 3H), 3.65 (s, 2H), 3.17 (s, 1H), 2.53-2.47 (m, 3H), 2.44 (d, J = 2.4 Hz, 3H), 2.25 (q, J = 6.9 Hz, 2H), 2.16-1.98 (m, 3H), 1.95-1.84 (m, 1H), 1.58 (t, J = 5.7 Hz, 4H), 0.93 (d, J = 4.6 Hz, 9H). |
| 1023 | 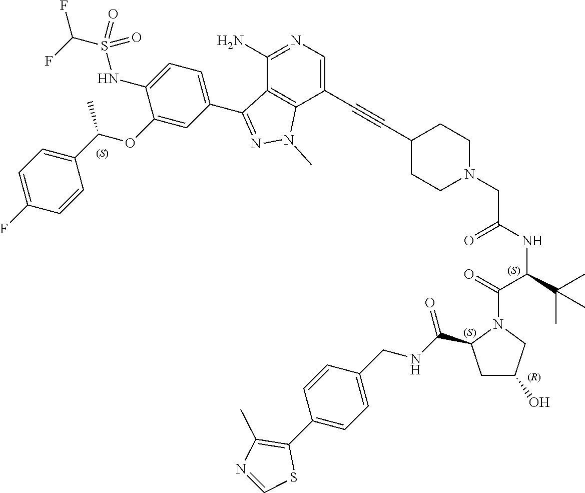 | (2S,4R)-1-[(2S)-2-{2-[4-(2-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}ethynyl)piperidin-1-yl] acetamido}-3,3-dimethylbutanoyl]-4-hydroxy-N-{[4-(4-methyl-1,3-thiazol-5-yl)phenyl]methyl} pyrrolidine-2-carboxamide From Intermediate G5. LCMS (method A): 2.93 min, m/z: 535.2 [M + 2H]²⁺. ¹H NMR (400 MHz, DMSO-d₆): 8.88 (s, 1H), 8.64-8.60 (m, 1H), 7.80 (s, 1H), 7.58-7.53 (m, 2H), 7.41-7.37 (m, 4H), 7.15 (t, J = 8.6 Hz, 2H), 7.09 (d, J = 10.2 Hz, 2H), 5.99 (s, 2H), 5.65 (d, J = 6.6 Hz, 1H), 5.18 (d, J = 3.1 Hz, 1H), 4.51 (d, J = 9.8 Hz, 1H), 4.45 (d, J = 7.6 Hz, 1H), 4.36 (d, J = 5.5 Hz, 2H), 4.29 (d, J = 7.4 Hz, 1H), 4.22 (s, 1H), 4.20 (s, 2H), 3.73-3.53 (m, 4H), 2.79 (s, 2H), 2.42 (s, 3H), 2.06-1.99 (m, 2H), 1.93 (d, J = 11.3 Hz, 2H), 1.77-1.70 (m, 2H), 1.56 (d, J = 5.9 Hz, 3H), 1.34 (d, J = 6.1 Hz, 2H), 0.95 (s, 9H). |
| 1024 | 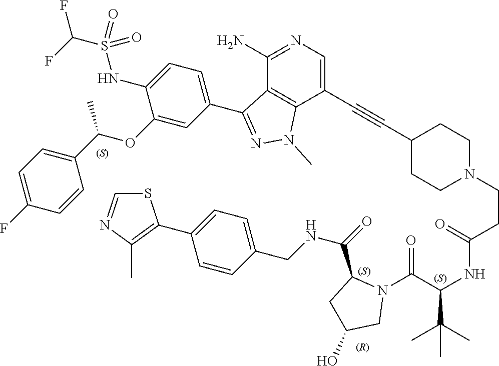 | (2S,4R)-1-[(2S)-2-{3-[4-(2-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}ethynyl) piperidin-1-yl]propanamido}-3,3-dimethylbutanoyl]-4-hydroxy-N-{[4-(4-methyl-1,3-thiazol-5-yl) phenyl]methyl}pyrrolidine-2-carboxamide From Intermediate G6. LCMS (method A): 4.0 min, m/z: 542.0 [M + 2H]²⁺. ¹H NMR (400 MHz, DMSO-d₆): 10.60 (s, 1H), 8.94 (s, 1H), 8.61 (t, J = 5.9 Hz, 2H), 7.79 (s, 1H), 7.58-7.55 (m, 2H), 7.41-7.38 (m, 4H), 7.19- 7.14 (m, 2H), 7.11-7.06 (m, 2H), 6.87 (s, 1H), 6.00 (s, 2H), 5.67 (q, J = 6.3 Hz, 1H), 5.19 (d, J = 3.4 Hz, 1H), 4.58 (d, J = 9.4 Hz, 1H), 4.51-4.36 (m, 4H), 4.21 (s, 3H), 3.75-3.57 (m, 3H), 3.19 (s, 1H), 3.03 (s, 2H), 2.85 (s, 2H), 2.58 (s, 3H), 2.44 (s, 3H), 2.05 (t, J = 7.4 Hz, 4H), 1.97-1.76 (m, 4H), 1.58 (d, J = 6.2 Hz, 4H), 1.36 (d, J = 6.2 Hz, 1H), 0.98 (s, 9H). |
| 1063 | 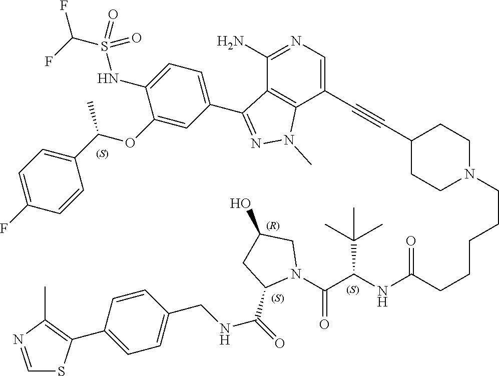 | (2S,4R)-1-[(2S)-2-{5-[4-(2-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}ethynyl) piperidin-1-yl]pentanamido}-3,3-dimethylbutanoyl]-4-hydroxy-N-{[4-(4-methyl-1,3-thiazol-5- yl)phenyl]methyl}pyrrolidine-2-carboxamide From Intermediate G7. LCMS (Method A): 2.55 min, m/z: 556.3 [M + 2H]²⁺. ¹H NMR (400 MHz, DMSO-d₆): 8.98 (s, 1H), 8.56 (t, J = 6.0 Hz, 2H), 8.36 (s, 1H), 7.92 (d, J = 9.3 Hz, 2H), 7.84 (s, 1H), 7.54 (dd, J = 8.5, 5.6 Hz, 2H), 7.40 (q, J = 8.2 Hz, 6H), 7.16 (d, J = 8.9 Hz, 1H), 7.07 (d, J = 39.8 Hz, 2H), 6.01 (s, 1H), 5.75 (s, 1H), 5.65 (q, J = 6.4 Hz, 1H), 5.14 (d, J = 3.5 Hz, 1H), 4.54 (d, J = 9.3 Hz, 2H), 4.35 (s, 1H), 4.26-4.16 (m, 3H), 4.16-3.96 (m, 2H), 3.65 (s, 2H), 3.17 (d, J = 5.1 Hz, 2H), 2.44 (s, 3H), 2.29 (d, J = 7.9 Hz, 2H), 2.19 (dd, J = 13.5, 6.2 Hz, 2H), 2.07 (s, 2H), 2.03-1.92 (m, 2H), 1.91- 1.83 (m, 2H), 1.55 (d, J = 6.3 Hz, 2H), 1.30 (s, 2H), 1.26 (s, 2H), 1.21 (s, 3H), 0.94 (s, 9H). |

### Compound 1052: (2S,4R)-1-[(2S)-2-(9-{4-amino-3-[4-(difluoromethanesulfonamido)-3-fluorophenyl]-1-methyl-1H-pyrazolo[4,3-c]pyridin-7-yl}non-8-ynamido)-3,3-dimethylbutanoyl]-4-hydroxy-N-{[4-(4-methyl-1,3-thiazol-5-yl)phenyl]methyl}pyrrolidine-2-carboxamide

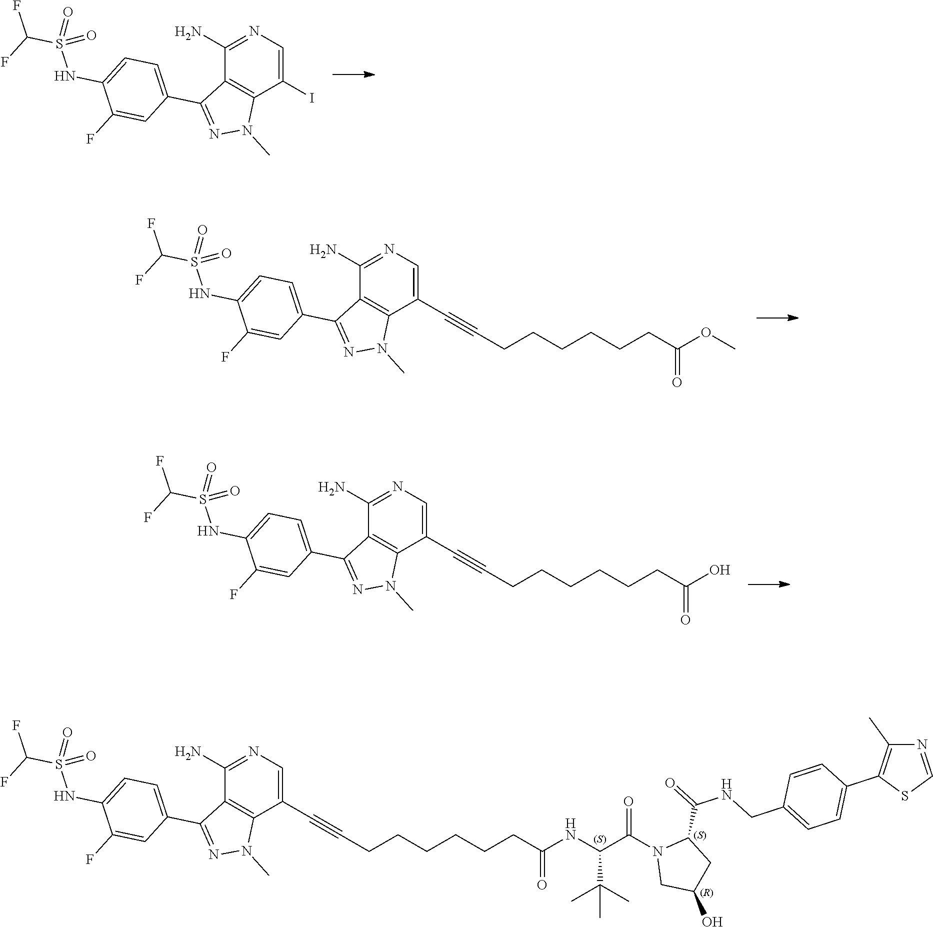

### Step 1: methyl 9-{4-amino-3-[4-(difluoromethanesulfonamido)-3-fluorophenyl]-1-methyl-1H-pyrazolo[4,3-c]pyridin-7-yl}non-8-ynoate

A mixture of N-(4-{4-amino-7-iodo-1-methyl-1H-pyrazolo[4,3-c]pyridin-3-yl}-2-fluorophenyl)-1,1-difluoromethanesulfonamide (200 mg, 402 μmol), methyl non-8-ynoate (67.6 mg, 402 μmol) Pd(PPh₃)₂Cl₂ (28.2 mg, 40.2 μmol) Cul (11.4 mg, 60.2 μmol) and Et₃N (121 mg, 1.20 mmol) in DMF (3 mL) was stirred at 90° C. under N₂ overnight. The mixture was concentrated, and the residue was poured into water and extracted with EtOAc. The combined organic phases were dried over Na₂SO₄ and concentrated. The residue was purified by Prep-TLC (DCM/MeOH=15/1) to afford the title compound (210 mg, 97%) as a yellow solid. LCMS (Method A): 4.25 min; m/z: 538.0 [M+H]⁺.

### Step 2: 9-{4-amino-3-[4-(difluoromethanesulfonamido)-3-fluorophenyl]-1-methyl-1H-pyrazolo [4,3-c]pyridin-7-yl}non-8-ynoic acid

A mixture of methyl 9-[4-amino-3-(4-ethanesulfonamido-3-fluorophenyl)-1-methyl-1H-pyrazolo[4,3-c]pyridin-7-yl]non-8-ynoate (210 mg, 407 μmol) and 1M NaOH (2 mL) in MeOH (2 mL) was stirred at RT for 2 h. The mixture was concentrated, and the residue was poured into water and extracted with EtOAc to afford the title compound (170 mg, 83%) as a yellow oil.

### Step 3: (2S,4R)-1-[(2S)-2-(9-{4-amino-3-[4-(difluoromethanesulfonamido)-3-fluorophenyl]-1-methyl-1H-pyrazolo[4,3-c]pyridin-7-yl}non-8-ynamido)-3,3-dimethylbutanoyl]-4-hydroxy-N-{[4-(4-methyl-1,3-thiazol-5-yl)phenyl]methyl}pyrrolidine-2-carboxamide

A mixture of 9-{4-amino-3-[4-(difluoromethanesulfonamido)-3-fluorophenyl]-1-methyl-1H-pyrazolo[4,3-c]pyridin-7-yl}non-8-ynoic acid (170 mg, 324 μmol), HATU (147 mg, 388 μmol), Et₃N (98.2 mg, 971 μmol) and (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide hydrochloride (172 mg, 324 μmol) in DMF (3 mL) was stirred at 100° C. under N₂ overnight. The mixture was concentrated, and the residue was poured into water and extracted with EtOAc. The combined organic phases were dried over Na₂SO₄ and concentrated. The residue was purified by Prep-TLC (DCM/MeOH=10/1) to afford the title compound (23 mg, 8%) as a yellow solid. LCMS (Method A): 4.25 min, m/z: 937.8 [M+H]⁺. ¹H NMR (400 MHz, DMSO-d₆): 8.98 (s, 1H), 8.56 (t, J=6.1 Hz, 1H), 7.87 (d, J=9.3 Hz, 1H), 7.80 (s, 1H), 7.48-7.43 (m, 2H), 7.40 (d, J=7.2 Hz, 2H), 7.17-7.07 (m, 2H), 6.31 (t, J=54.7 Hz, 2H), 6.04 (s, 2H), 5.13 (d, J=3.6 Hz, 1H), 4.55 (d, J=9.4 Hz, 1H), 4.43 (dd, J=9.9, 6.5 Hz, 2H), 4.37-4.33 (m, 1H), 4.23 (s, 3H), 4.20 (d, J=5.1 Hz, 1H), 3.67 (d, J=4.6 Hz, 2H), 2.55 (s, 3H), 2.45 (s, 3H), 2.29 (dd, J=14.3, 7.4 Hz, 1H), 2.19-2.12 (m, 1H), 2.02 (d, J=7.7 Hz, 1H), 1.91 (td, J=8.5, 4.4 Hz, 1H), 1.60-1.55 (m, 2H), 1.47 (t, J=5.0 Hz, 2H), 1.31 (d, J=7.5 Hz, 2H), 1.24 (d, J=3.6 Hz, 2H), 0.94 (s, 9H).

### Compound 1046: (2S,4R)-1-[(2S)-2-(9-{4-amino-3-[4-(difluoromethanesulfonamido)-3-fluorophenyl]-1-methyl-1H-pyrazolo[4,3-c]pyridin-7-yl}non-8-ynamido)-3,3-dimethylbutanoyl]-4-hydroxy-N-{[4-(4-methyl-1,3-thiazol-5-yl)phenyl]methyl}pyrrolidine-2-carboxamide

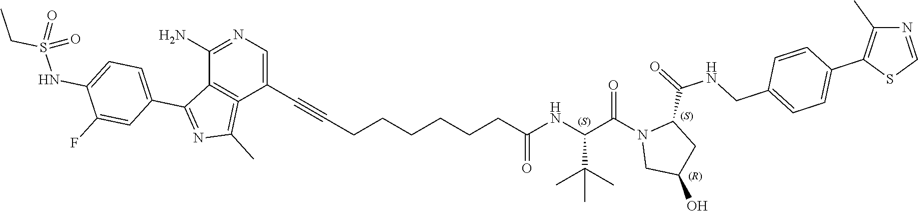

The title compound was prepared according to the procedure for the synthesis of Compound 1052, using Intermediate A1B20 in step 1. LCMS (Method A): 4.25 min, m/z: 938.0 [M+H]⁺. ¹H NMR (400 MHz, DMSO-d₆): 9.94 (s, 1H), 8.99 (s, 1H), 8.56 (t, J=6.1 Hz, 1H), 8.04 (s, 1H), 7.96 (s, 1H), 7.86 (d, J=9.3 Hz, 1H), 7.61 (d, J=8.3 Hz, 1H), 7.52 (dd, J=11.0, 2.0 Hz, 1H), 7.40 (q, J=8.3, 7.4 Hz, 5H), 4.55 (d, J=9.4 Hz, 2H), 4.44 (d, J=8.5 Hz, 3H), 4.35 (s, 3H), 3.70-3.62 (m, 2H), 3.25-3.17 (m, 2H), 2.55 (t, J=3.5 Hz, 2H), 2.45 (s, 3H), 2.34-2.25 (m, 1H), 2.14 (dt, J=14.2, 7.2 Hz, 1H), 2.03 (ddd, J=15.4, 7.7, 3.8 Hz, 1H), 1.91 (ddd, J=12.9, 8.6, 4.6 Hz, 1H), 1.60 (t, J=7.4 Hz, 2H), 1.52 (dt, J=10.6, 7.0 Hz, 2H), 1.44 (dd, J=10.6, 5.0 Hz, 2H), 1.32 (d, J=3.8 Hz, 2H), 1.29 (d, J=7.4 Hz, 3H), 0.94 (s, 9H).

### Compound 1075: (2S,4R)-1-[(2S)-2-(2-{2-[(3-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}prop-2-yn-1-yl)oxy]ethoxy}acetamido)-3,3-dimethylbutanoyl]-4-hydroxy-N-{[4-(4-methyl-1,3-thiazol-5-yl)phenyl]methyl}pyrrolidine-2-carboxamide

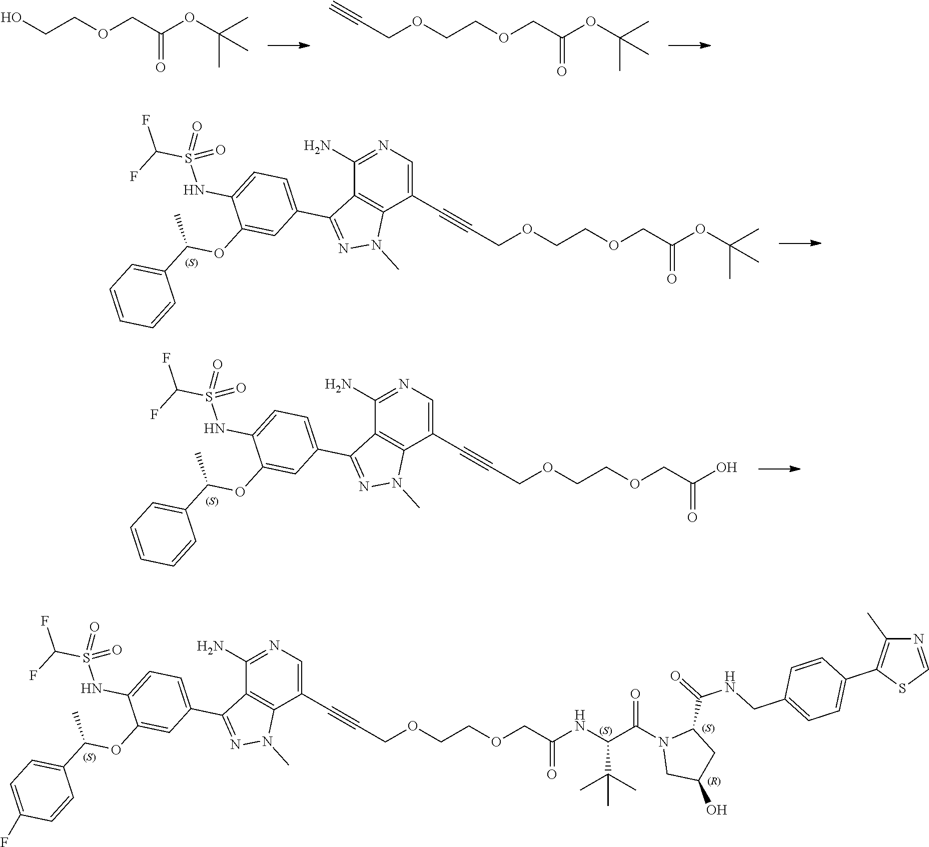

### Step 1: tert-butyl 2-[2-(prop-2-yn-1-yloxy)ethoxy]acetate

A mixture of tert-butyl 2-(2-hydroxyethoxy)acetate (100 mg, 0.57 mmol) and NaH (49.5 mg, 1.24 mmol) in THE (3 mL) was stirred at 0° C. for 0.5 h. 3-Bromoprop-1-yne (74.1 mg, 0.62 mmol) was then added slowly, and the reaction stirred overnight. The mixture was concentrated under reduced pressure and water was added. The mixture was filtered, and the filtrate was concentrated to afford the title compound (110 mg, 91%) as a yellow oil. ¹H NMR (400 MHz, CDCl₃): 4.22 (d, J=2.3 Hz, 2H), 4.03 (s, 2H), 3.74 (d, J=3.9 Hz, 4H), 2.43 (q, J=2.6 Hz, 1H), 1.48 (s, 9H).

### Step 2: tert-butyl 2-{2-[(3-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}prop-2-yn-1-yl)oxy]ethoxy}acetate

A mixture of tert-butyl 2-[2-(prop-2-yn-1-yloxy)ethoxy]acetate (100 mg, 466 μmol), N-(4-{4-amino-7-iodo-1-methyl-1H-pyrazolo[4,3-c]pyridin-3-yl}-2-[(1 S)-1-(4-fluorophenyl)ethoxy]phenyl)-1,1-difluoromethanesulfonamide (287 mg, 466 μmol), Pd(PPh₃)₂Cl₂ (32.7 mg, 46.6 μmol), Cul (13.2 mg, 69.8 μmol) and Et₃N (140 mg, 1.39 mmol) in DMF (3 mL) was stirred at 130° C. under N₂ overnight. The mixture was concentrated, and the residue was poured into water and extracted with EtOAc. The combined organic phases were dried over Na₂SO₄ and concentrated. The residue was purified by Prep-TLC (DCM/MeOH=20/1) to afford the title compound (220 mg, 67%) as a yellow solid. LCMS (Method A): 4.25 min, m/z: 704.2 [M+H]⁺.

### Step 3: 2-{2-[(3-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}prop-2-yn-1-yl)oxy]ethoxy}acetic acid

A solution of tert-butyl 2-{2-[(3-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}prop-2-yn-1-yl)oxy]ethoxy}acetate (220 mg, 320 μmol) in HCO₂H (2 mL) was stirred at RT for 2 h. The mixture was concentrated, and the residue was poured into water and extracted with EtOAc. The combined organic phases were dried over Na₂SO₄ and concentrated to afford the title compound (120 mg, 59%). LCMS (Method A): 4.25 min. m/z: 648.0 [M+H]⁺.

### Step 4: (2S,4R)-1-[(2S)-2-(2-{2-[(3-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}prop-2-yn-1-yl)oxy]ethoxy}acetamido)-3,3-dimethylbutanoyl]-4-hydroxy-N-{[4-(4-methyl-1,3-thiazol-5-yl)phenyl]methyl}pyrrolidine-2-carboxamide

A mixture of 2-{2-[(3-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}prop-2-yn-1-yl)oxy]ethoxy}acetic acid (150 mg, 231 μmol), (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide hydrochloride (107 mg, 231 μmol), HATU (105 mg, 277 μmol) and Et₃N (116 mg, 1.15 mmol) in MeCN (3 mL) was stirred at 0° C. under N₂ for 3 h. The mixture was concentrated, and the residue was poured into water and extracted with EtOAc. The combined organic phases were dried over Na₂SO₄ and concentrated. The residue was purified by Prep-TLC (DCM/MeOH=20/1) to afford the title compound (15 mg, 6%) as a yellow solid. LCMS (Method A): 4.25 min, m/z: 530.8 [M+2H]²⁺. ¹H NMR (400 MHz, DMSO-d₆): 10.54 (s, 1H), 8.88 (s, 1H), 8.60 (t, J=6.0 Hz, 1H), 8.13 (s, 1H), 7.92 (s, 1H), 7.59-7.53 (m, 3H), 7.41-7.38 (m, 4H), 7.18 (s, 1H), 7.17-7.12 (m, 4H), 7.03 (s, 1H), 6.68 (s, 2H), 5.64 (d, J=6.3 Hz, 1H), 5.16 (s, 1H), 4.54 (s, 3H), 4.47-4.41 (m, 2H), 4.38-4.34 (m, 1H), 4.26-4.23 (m, 1H), 4.21 (s, 3H), 4.00 (s, 2H), 3.69 (ddd, J=13.6, 10.4, 5.3 Hz, 6H), 2.40 (s, 3H), 2.06 (dd, J=13.0, 7.7 Hz, 1H), 1.90 (td, J=8.8, 4.5 Hz, 1H), 1.58 (d, J=6.3 Hz, 3H), 1.23 (d, J=3.4 Hz, 1H), 0.95 (s, 9H).

### Intermediate H: (2S,4R)-1-[(2S)-2-(5-bromopentanamido)-3,3-dimethylbutanoyl]-4-hydroxy-N-{[4-(4-methyl-1,3-thiazol-5-yl)phenyl]methyl}pyrrolidine-2-carboxamide

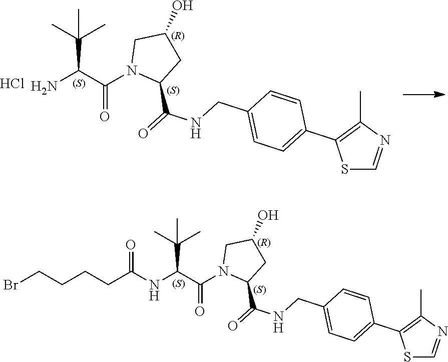

### Step 1: (2S,4R)-1-[(2S)-2-(5-bromopentanamido)-3,3-dimethylbutanoyl]-4-hydroxy-N-{[4-(4-methyl-1,3-thiazol-5-yl)phenyl]methyl}pyrrolidine-2-carboxamide

A mixture of (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide (100 mg, 232 μmol), 5-bromopentanoic acid (46.1 mg, 255 μmol), Et₃N (70.4 mg, 696 μmol) and HATU (105 mg, 278 μmol) was stirred at 25° C. under N₂ for 16 h. The mixture was filtered through Celite, and the filter cake was washed with MeOH. The filtrate was concentrated under reduced pressure and the crude residue was purified by column chromatography (PE:EtOAc, 3:1) to afford the title compound (140 mg, 102%) as a yellow solid. LCMS (method A): 3.81 min; m/z: 593.58 [M+H]⁺.

### 1064: 4-{[(2S)-1-[(2S,4R)-4-hydroxy-2-({[4-(4-methyl-1,3-thiazol-5-yl)phenyl]methyl}carbamoyl)pyrrolidin-1-yl]-3,3-dimethyl-1-oxobutan-2-yl]carbamoyl}butyl 4-{4-amino-3-[4-(difluoromethanesulfonamido)-3-[(1 S)-1-(4-fluorophenyl)ethoxy]phenyl]-1-methyl-1H-pyrazolo[4,3-c]pyridin-7-yl}benzoate

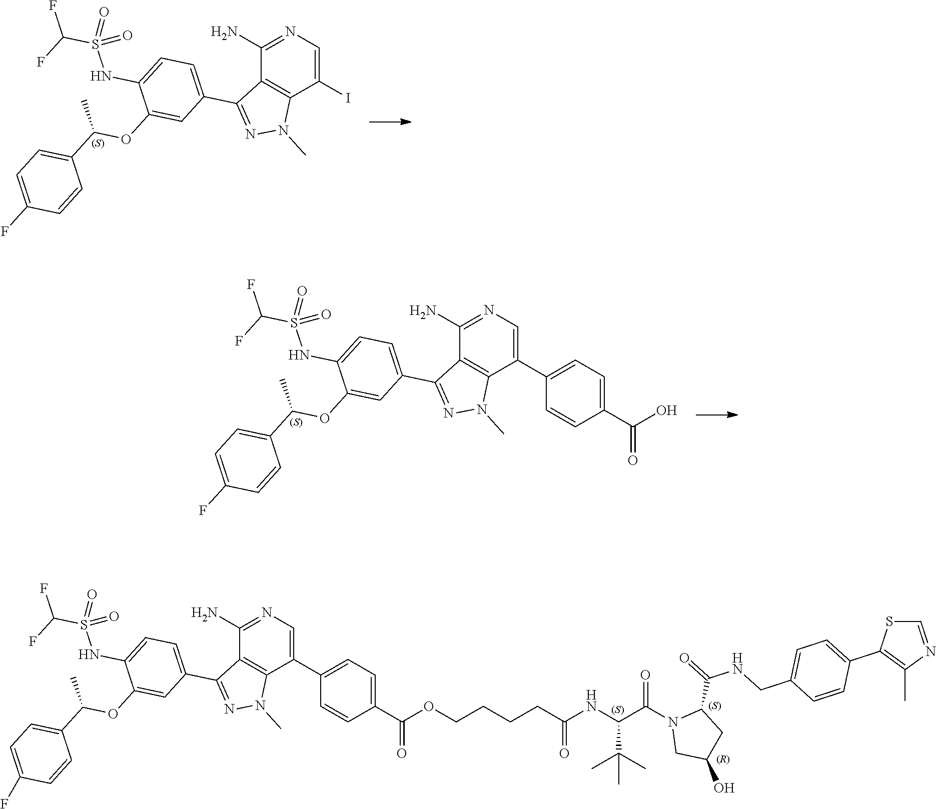

### Step 1: 4-{4-amino-3-[4-(difluoromethanesulfonamido)-3-[(1 S)-1-(4-fluorophenyl)ethoxy]phenyl]-1-methyl-1H-pyrazolo[4,3-c]pyridin-7-yl}benzoic acid

A mixture of N-(4-{4-amino-7-iodo-1-methyl-1H-pyrazolo[4,3-c]pyridin-3-yl}-2-[(1S)-1-(4-10 fluorophenyl)ethoxy]phenyl)-1,1-difluoromethanesulfonamide (100 mg, 161 μmol), 4-(dihydroxyboranyl)benzoic acid (39.9 mg, 241 μmol), Pd(PPh₃)₄ (18.4 mg, 16.0 μmol) and Na₂CO₃ (51.0 mg, 482 μmol) in 75% aq. 1,4-Dioxane (2 mL) was stirred at 100° C. for 2 h under N₂. Once completed, the mixture was diluted with water and extracted with MeOH/DCM=1/10. The organics were dried over Na₂SO₄ and concentrated. The residue was purified by 15 Prep-TLC (MeOH/DCM=1/15) to afford the title compound (40.0 mg, 41%). LCMS (Method A): 3.47 min, m/z: 612.2 [M+H]⁺.

### Step 2: 4-{[(2S)-1-[(2S,4R)-4-hydroxy-2-({[4-(4-methyl-1,3-thiazol-5-yl)phenyl]methyl}carbamoyl)pyrrolidin-1-yl]-3,3-dimethyl-1-oxobutan-2-yl]carbamoyl}butyl 4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}benzoate

A mixture of 4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}benzoic acid (30 mg, 49.0 μmol), (2S,4R)-1-[(2S)-2-(5-bromopentanamido)-3,3-dimethylbutanoyl]-4-hydroxy-N-{[4-(4-methyl-1,3-thiazol-5-yl)phenyl]methyl}pyrrolidine-2-carboxamide (34.9 mg, 58.8 μmol) and Cs₂CO₃ (47.9 mg, 147 μmol) in DMF (2 mL) was stirred at 80° C. for 2 h under N₂. Once completed, the mixture was diluted with water and extracted with MeOH/DCM=1/10. The organics were dried over Na₂SO₄ and concentrated. The residue was purified by Prep-TLC (MeOH/DCM=1/15) to afford the title compound (22.0 mg, 40%) as yellow solid. LCMS (Method A): 2.83 min, m/z: 1124.3 [M+H]⁺. ¹HNMR (400 MHz, DMSO-d₆): 10.62 (s, 1H), 8.97 (s, 1H), 8.55 (d, J=6.3 Hz, 1H), 8.06 (s, 1H), 7.93 (d, J=9.3 Hz, 1H), 7.64-7.14 (m, 14H), 7.02 (s, 1H), 6.89 (s, 1H), 5.91 (s, 2H), 5.66 (d, J=7.3 Hz, 1H), 5.13 (s, 1H), 4.56 (d, J=9.2 Hz, 1H), 4.48-4.38 (m, 2H), 4.34-4.30 (m, 2H), 4.26-4.19 (m, 1H), 3.66 (s, 1H), 3.56 (s, 3H), 2.44 (s, 3H), 2.37-2.21 (m, 2H), 2.02 (d, J=9.2 Hz, 2H), 1.90 (s, 2H), 1.58 (d, J=6.2 Hz, 2H), 1.43-1.36 (m, 2H), 1.25 (s, 3H), 0.94 (s, 9H).

### Compound 1065: 4-{[(2S)-1-[(2S,4R)-4-hydroxy-2-({[4-(4-methyl-1,3-thiazol-5-yl)phenyl]methyl}carbamoyl)pyrrolidin-1-yl]-3,3-dimethyl-1-oxobutan-2-yl]carbamoyl}butyl 5-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}pyridine-2-carboxylate

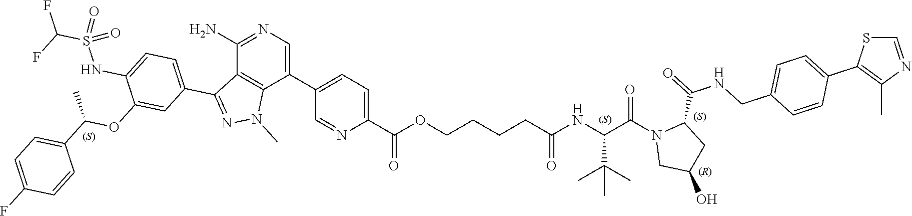

The title compound was prepared according to the procedure for the synthesis of Compound 1064, utilising 5-(4,4,5,5-tetramethyl-1,3,2-dioxaborolan-2-yl)pyridine-2-carboxlic acid in Step 1. LCMS (Method A): 2.08 min, 1125.3 [M+H]⁺. ¹HNMR (400 MHz, DMSO-d₆): 10.56 (s, 1H), 8.98 (d, J=4.2 Hz, 1H), 8.84 (s, 1H), 8.56 (t, J=6.1 Hz, 1H), 8.12 (q, J=8.2 Hz, 2H), 7.94 (d, J=9.4 Hz, 2H), 7.67 (s, 2H), 7.57 (dd, J=8.4, 5.4 Hz, 2H), 7.40 (dd, J=10.4, 7.3 Hz, 6H), 7.16 (d, J=7.5 Hz, 2H), 7.12 (s, 1H), 7.01-6.89 (m, 1H), 5.94 (s, 1H), 5.66 (q, J=6.5 Hz, 1H), 5.13 (d, J=3.6 Hz, 1H), 4.59-4.44 (m, 2H), 4.35 (d, J=5.4 Hz, 2H), 4.23 (d, J=5.4 Hz, 1H), 3.66 (d, J=5.0 Hz, 1H), 3.59 (s, 3H), 2.23 (dd, J=14.1, 7.9 Hz, 2H), 2.03 (t, J=10.4 Hz, 2H), 1.96-1.76 (m, 2H), 1.58 (d, J=6.2 Hz, 2H), 1.23 (s, 3H), 0.94 (s, 9H).

### Compound 1104: (2S,4R)—N-(2-{2-[4-(4-{4-amino-3-[4-(difluoromethanesulfonamido)-3-[(1 S)-1-(4-fluorophenyl)ethoxy]phenyl]-1-methyl-1H-pyrazolo[4,3-c]pyridin-7-yl}-1H-pyrazol-1-yl)piperidin-1-yl]-2-oxoethoxy}-1-[4-(4-methyl-1,3-thiazol-5-yl)phenyl]ethyl)-1-[(2S)-2-[(1-fluorocyclopropyl)formamido]-3,3-dimethylbutanoyl]-4-hydroxypyrrolidine-2-carboxamide

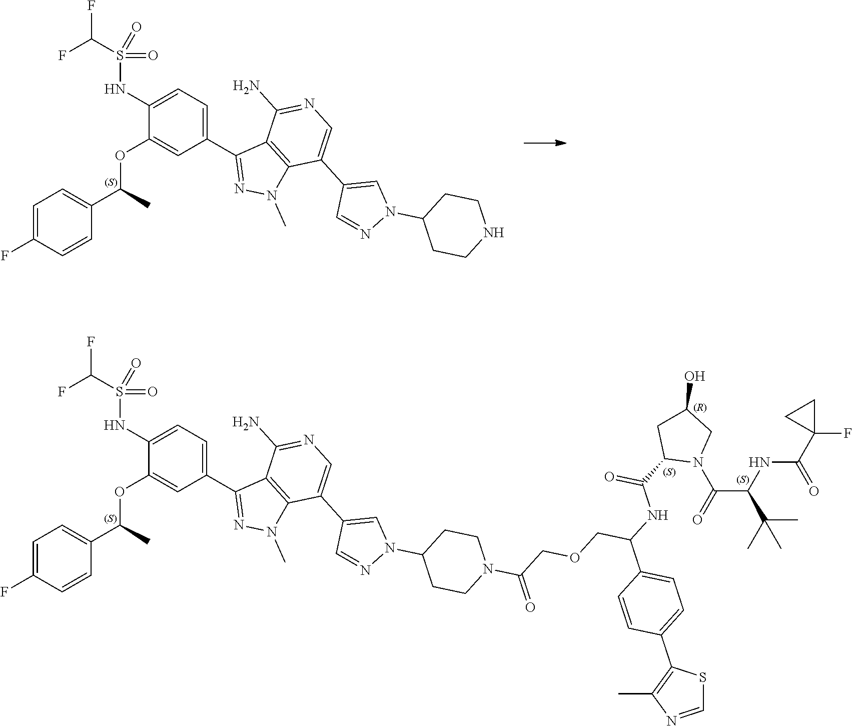

To a solution of 2-(2-{[(2S,4R)-1-[(2S)-2-[(1-fluorocyclopropyl)formamido]-3,3-dimethyl butanoyl]-4-hydroxypyrrolidin-2-yl]formamido}-2-[4-(4-methyl-1,3-thiazol-5-yl)phenyl]ethoxy) acetic acid (100 mg, 165.3 μmol) in DMF (10 mL) were added N-(4-{4-amino-1-methyl-7-[1-(piperidin-4-yl)-1H-pyrazol-4-yl]-1H-pyrazolo[4,3-c]pyridin-3-yl}-2-[(1 S)-1-(4-fluorophenyl) ethoxy]phenyl)-1,1-difluoromethanesulfonamide (127 mg, 198.3 μmol), HATU (100 mg, 264.4 μmol) and DIPEA (142 μL, 826.5 μmol). The reaction was stirred at RT overnight, then concentrated in vacuo. The residue was diluted with brine (15 mL) and extracted with EtOAc (3×15 mL). The combined organics were dried over MgSO₄ and concentrated. The residue was purified by flash chromatography (0-15% MeOH:DCM) and Prep-HPLC to afford the title compound (14.1 mg, 7%) as an off-white solid. LCMS (Method H): 2.07 min, m/z: 1229.2 [M+H]⁺. ¹H NMR (300 MHz, DMSO-d₆): 12.58 (bs, 1H), 10.60 (bs, 1H), 8.97 (s, 1H), 8.08 (s, 1H), 7.72 (d, J=1.9 Hz, 1H), 7.66-7.52 (m, 4H), 7.43 (m, 4H), 7.29 (d, J=7.3 Hz, 2H), 7.25-7.11 (m, 4H), 7.05 (t, J=52.5 Hz, 1H), 5.65 (q, J=6.2 Hz, 1H), 5.19 (bs, 1H), 4.99 (bs, 1H), 4.54 (dd, J=24.4, 8.7 Hz, 4H), 4.37-4.28 (m, 3H), 3.84 (d, J=12.2 Hz, 1H), 3.79-3.68 (m, 4H), 3.62 (s, 2H), 2.83 (t, J=12.0 Hz, 1H), 2.55 (s, 2H), 2.45 (d, J=3.5 Hz, 3H), 2.13-2.04 (m, 3H), 1.95-1.78 (m, 3H), 1.60 (d, J=6.2 Hz, 3H), 1.36 (dd, J=18.5, 4.3 Hz, 2H), 1.23 (dd, J=7.0, 3.8 Hz, 2H), 0.98 (s, 4.5H), 0.91 (s, 4.5H).

### Compound 1138: (2S,4R)—N-[(2-{[9-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl) nonyl]oxy}-4-(4-methyl-1,3-thiazol-5-yl)phenyl)methyl]-1-[(2S)-2-[(1-fluorocyclopropyl) formamido]-3,3-dimethylbutanoyl]-4-hydroxypyrrolidine-2-carboxamide

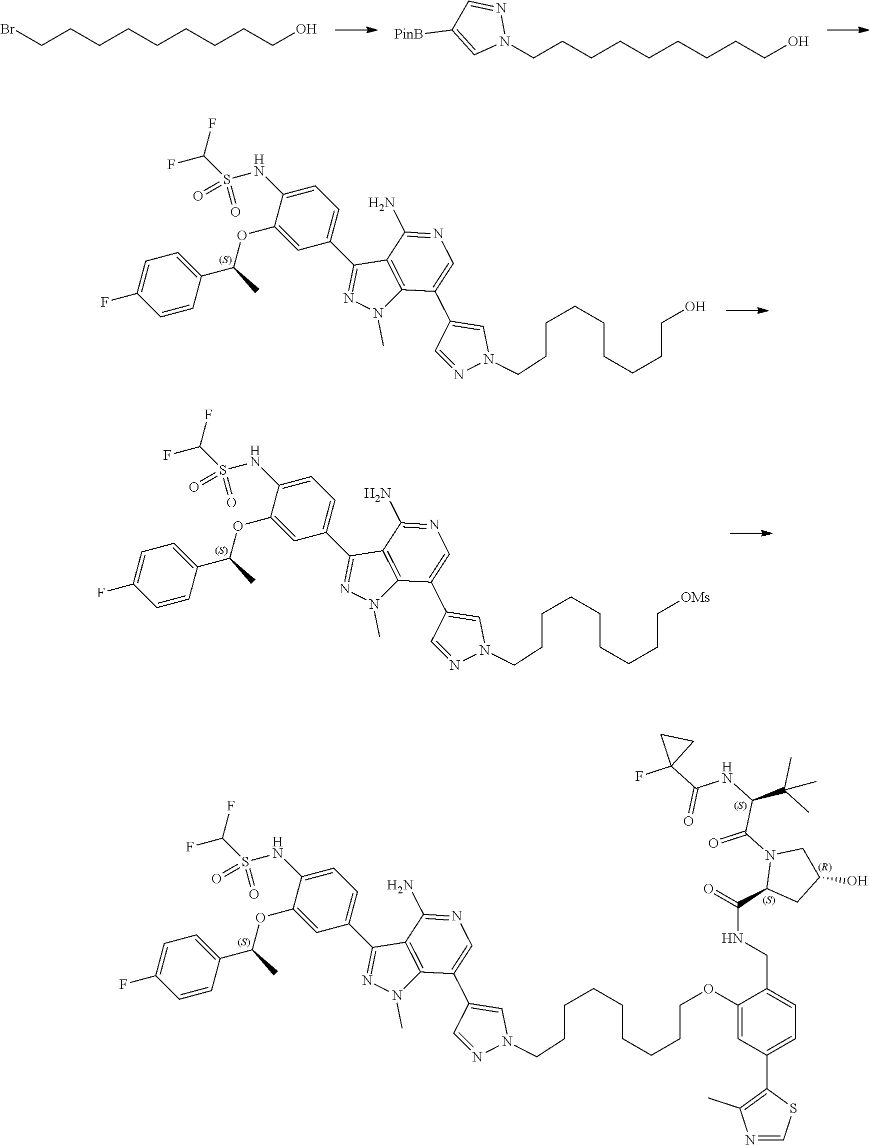

### Step 1: 9-[4-(4,4,5,5-tetramethyl-1,3,2-dioxaborolan-2-yl)-1H-pyrazol-1-yl]nonan-1-ol

A mixture of 9-bromononan-1-ol (2 g, 8.96 mmol), 4-(4,4,5,5-tetramethyl-1,3,2-dioxaborolan-2-yl)-1H-pyrazole (1.04 g, 5.37 mmol) and Cs₂CO₃ (5.83 g, 17.9 mmol) in MeCN (30 mL) was heated at 80° C. under N₂ for 16 h. The mixture was diluted with H₂O (100 mL) and extracted with EtOAc (3×100 mL). The combined organic layers were dried (Na₂SO₄) and then concentrated. The crude residue was purified by column chromatography (DCM:MeOH, 250:1) to afford the title product (1.1 g, 73%) as a yellow solid. LCMS (method A): 3.56 min; m/z: 337.2 [M+H]⁺.

### Step 2: N-(4-{4-amino-7-[1-(9-hydroxynonyl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

A mixture of 9-[4-(4,4,5,5-tetramethyl-1,3,2-dioxaborolan-2-yl)-1H-pyrazol-1-yl]nonan-1-ol (1 g, 2.37 mmol), N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (1.46 g, 2.37 mmol), Na₂CO₃ (502 mg, 4.74 mmol) and Pd(dppf)Cl₂ (96.3 mg, 118 μmol) in 80% aq. 1,4-dioxane (20 mL) was heated at 100° C. overnight. The mixture was diluted with H₂O (50 mL) and extracted with EtOAc (3×40 mL). The combined organic layers were washed (brine), dried (Na₂SO₄) and concentrated. The residue was purified by column chromatography (DCM:MeOH, 200:1) to afford the title product (891 mg, 54%) as a yellow oil. LCMS (method A): 2.95 min; m/z: 700.2 [M+H]⁺.

### Step 3: 9-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl-}1H-pyrazol-1-yl)nonyl methanesulfonate

A mixture of N-(4-{4-amino-7-[1-(9-hydroxynonyl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide (400 mg, 571 μmol), Et₃N (115 mg, 1.14 mmol) and MsCl (78.4 mg, 685 μmol) in DCM (15 mL) was stirred at rt overnight. The mixture was concentrated and the residue was purified by prep-TLC (DCM:MeOH, 20:1) to afford the title product (100 mg, 22%) as a yellow solid. LCMS (method B): 4.01 min; m/z: 777.9 [M+H]+.

### Step 4: (2S,4R)—N-[(2-{[9-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)nonyl]oxy}-4-(4-methyl-1,3-thiazol-5-yl)phenyl)methyl]-1-[(2S)-2-[(1-fluorocyclopropyl)formamido]-3,3-dimethylbutanoyl]-4-hydroxypyrrolidine-2-carboxamide

A mixture of 9-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)nonyl methanesulfonate (100 mg, 128 μmol), (2S,4R)-1-[(2S)-2-[(1-fluorocyclopropyl)formamido]-3,3-dimethylbutanoyl]-4-hydroxy-N-{[2-hydroxy-4-(4-methyl-1,3-thiazol-5-yl)phenyl]methyl}pyrrolidine-2-carboxamide (68.1 mg, 128 μmol) and Cs₂CO₃ (83.4 mg, 256 μmol) in NMP (3 mL) was stirred at 105° C. under N₂ for 16 h. The mixture was diluted with H₂O (30 mL) and then extracted with EtOAc (3×20 mL). The combined organic layers were washed (brine), dried (Na₂SO₄) and concentrated under reduced pressure. The crude residue was purified by prep-TLC (DCM:MeOH, 20:1) to afford the title product (8 mg, 5%) as a yellow solid. LCMS (method A): 3.19 min; 1215.4 [M+H]⁺. ¹H NMR (400 MHz, DMSO-d₆): 8.97 (s, 1H), 8.57 (s, 1H), 7.96 (s, 1H), 7.59 (s, 1H), 7.56 (s, 2H), 7.40 (d, J=6.2 Hz, 2H), 7.28 (d, J=8.1 Hz, 1H), 7.14 (m, 4H), 6.96 (m, 3H), 6.88 (t, J=85.2 Hz, 1H), 6.26 (br s, 2H), 5.65 (q, J=5.6 Hz, 1H), 5.27 (s, 1H), 4.58 (d, J=9.1 Hz, 1H), 4.52 (t, J=8.3 Hz, 1H), 4.34 (s, 1H), 4.31-4.19 (m, 2H), 4.09 (dt, J=44.9, 6.7 Hz, 4H), 3.70 (s, 3H), 3.60 (t, J=10.3 Hz, 2H), 2.44 (s, 3H), 1.97 (ddd, J=44.8, 24.2, 9.9 Hz, 4H), 1.77 (dt, J=28.7, 6.9 Hz, 4H), 1.57 (d, J=6.2 Hz, 3H), 1.49-1.28 (m, 12H), 0.94 (s, 9H).

The following examples were similarly prepared from the appropriate halo-alcohol according to the procedure for the synthesis of Compound 1138.

TABLE 15
Compounds 1045, 1057, 1061, 1068 and 1041

| Example | Structure | Compound Name and Analytical Data |
|---|---|---|
| 1045 | 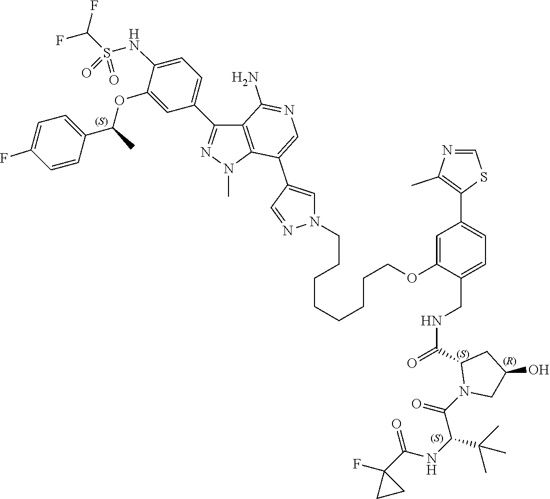 | (2S,4R)-N-[(2-{[8-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1- yl)octyl]oxy}-4-(4-methyl-1,3-thiazol-5-yl)phenyl)methyl]-1-[(2S)-2-[(1- fluorocyclopropyl)formamido]-3,3-dimethylbutanoyl]-4-hydroxypyrrolidine-2-carboxamide From 8-bromooctan-1-ol. LCMS (method A): 3.17 min, m/z: 600.9 [M + 2H]²⁺. ¹H NMR (400 MHz, DMSO-d₆): 8.97 (s, 1H), 8.49 (t, J = 6.0 Hz, 1H), 8.14 (s, 1H), 7.91 (s, 1H), 7.57-7.52 (m, 3H), 7.51 (s, 1H), 7.39 (dd, J = 7.9, 6.0 Hz, 2H), 7.28 (dd, J = 9.3, 2.8 Hz, 1H), 7.15 (t, J = 8.9 Hz, 2H), 7.08-7.01 (m, 2H), 6.99 (d, J = 1.7 Hz, 1H), 6.94 (dd, J = 7.8, 1.6 Hz, 1H), 6.79 (s, 1H), 6.65 (s, 1H), 5.65 (d, J = 5.9 Hz, 3H), 5.16 (s, 1H), 4.59 (d, J = 9.3 Hz, 1H), 4.51 (t, J = 8.2 Hz, 1H), 4.34 (s, 1H), 4.27 (d, J = 6.1 Hz, 1H), 4.22 (d, J = 5.6 Hz, 1H), 4.15 (t, J = 7.1 Hz, 2H), 4.04 (t, J = 6.3 Hz, 2H), 3.68 (s, 3H), 3.64-3.56 (m, 2H), 2.45 (s, 3H), 2.08 (t, J = 10.2 Hz, 1H), 1.95-1.89 (m, 1H), 1.83 (t, J = 7.1 Hz, 2H), 1.75 (t, J = 7.2 Hz, 2H), 1.55 (d, J = 6.3 Hz, 3H), 1.45 (s, 2H), 1.34 (dd, J = 6.5, 3.5 Hz, 6H), 1.28-1.19 (m, 6H), 0.95 (s, 9H). |
| 1057 | 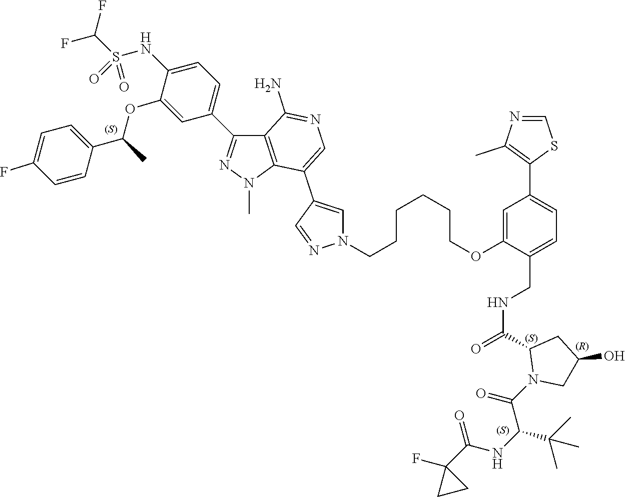 | (2S,4R)-N-[(2-{[6-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1- yl)hexyl]oxy}-4-(4-methyl-1,3-thiazol-5-yl)phenyl)methyl]-1-[(2S)-2-[(1- fluorocyclopropyl)formamido]-3,3-dimethylbutanoyl]-4-hydroxypyrrolidine-2-carboxamide From 6-bromohexan-1-ol. LCMS (method A): 3.01 min, m/z 1172.0 [M + H]⁺. ¹H NMR (400 MHz, DMSO-d₆): 8.72, 7.74 (d, J = 0.8 Hz), 7.54-7.49 (m), 7.47-7.32 (m), 7.07 (dd, J = 8.1, 1.9 Hz), 7.01 (d, J = 1.8 Hz), 6.86 (dq, J = 4.2, 1.8 Hz), 6.60, 5.48 (q, J = 6.3 Hz), 4.63 (d, J = 1.3 Hz), 4.52 (t, J = 8.3 Hz), 4.42-4.34 (m), 4.17 (t, J = 6.9 Hz), 2.14-2.07 (m), 2.01 (dd, J = 9.0, 4.4 Hz), 1.90-1.83 (m), 1.76 (q, J = 7.0 Hz), 1.60 (d, J = 6.4 Hz), 1.34 (d, J = 3.5 Hz), 1.32- 1.29 (m), 1.29-1.25 (m), 1.25-1.21 (m), 1.13 (d, J = 7.2 Hz), 0.91. |
| 1061 | 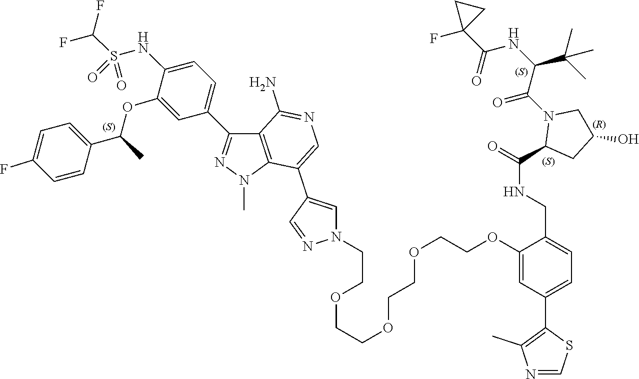 | (2S,4R)-N-({2-[2-(2-{2-[2-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1- yl)ethoxy]ethoxy}ethoxy)ethoxy]-4-(4-methyl-1,3-thiazol-5-yl)phenyl}methyl)-1-[(2S)-2- [(1-fluorocyclopropyl)formamido]-3,3-dimethylbutanoyl]-4-hydroxypyrrolidine-2- carboxamide From 2-{2-[2-(2-chloroethoxy)ethoxy]ethoxy}ethan-1-ol. LCMS (method A): 4.25 min, m/z 1249 [M + H]⁺. ¹H NMR (400 MHz, DMSO-d₆): 8.96 (s, 1H), 8.49 (t, J = 6.0 Hz, 1H), 8.14 (s, 1H), 7.90 (s, 1H), 7.59 (s, 1H), 7.56-7.53 (m, 2H), 7.39 (dd, J = 7.9, 5.6 Hz, 2H), 7.29 (dd, J = 9.3, 2.9 Hz, 1H), 7.18-7.12 (m, 2H), 7.11- 7.06 (m, 2H), 7.01 (d, J = 1.6 Hz, 1H), 6.95 (dd, J = 7.7, 1.6 Hz, 1H), 5.68 (s, 1H), 5.64 (d, J = 6.4 Hz, 1H), 4.59 (d, J = 9.1 Hz, 1H), 4.51 (t, J = 8.2 Hz, 1H), 4.34 (s, 1H), 4.29 (d, J = 4.9 Hz, 2H), 4.18-4.12 (m, 2H), 3.80 (t, J = 5.4 Hz, 2H), 3.72 (dd, J = 5.8, 3.3 Hz, 2H), 3.69 (s, 3H), 3.65-3.60 (m, 2H), 3.57 (dd, J = 6.2, 3.5 Hz, 2H), 3.52 (d, J = 9.9 Hz, 8H), 2.44 (s, 3H), 2.08 (dd, J = 12.8, 7.8 Hz, 1H), 1.91 (ddd, J = 13.0, 8.9, 4.3 Hz, 1H), 1.57 (d, J = 6.3 Hz, 3H), 1.40-1.32 (m, 2H), 1.24-1.21 (m, 2H), 0.95 (s, 9H). |
| 1068 | 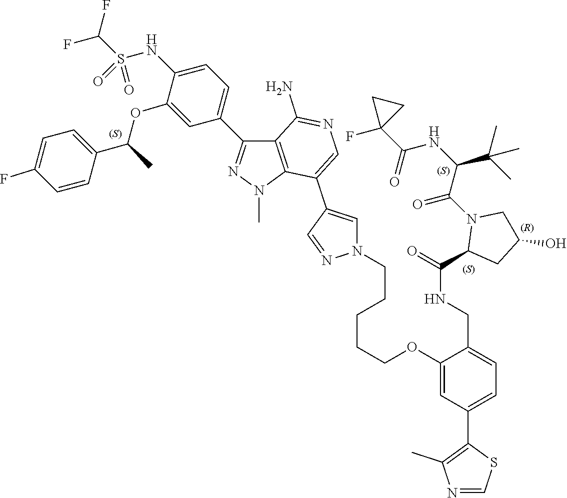 | (2S,4R)-N-[(2-{[5-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1- yl)pentyl]oxy}-4-(4-methyl-1,3-thiazol-5-yl)phenyl)methyl]-1-[(2S)-2-[(1- fluorocyclopropyl)formamido]-3,3-dimethylbutanoyl]-4-hydroxypyrrolidine-2-carboxamide From 5-bromopentan-1-ol LCMS (Method A): 4.25 min, m/z: 1249 [M + H]⁺. ¹H NMR (400 MHz, DMSO-d₆): 8.96 (s, 1H), 8.49 (t, J = 6.0 Hz, 1H), 8.14 (s, 1H), 7.90 (s, 1H), 7.59 (s, 1H), 7.56-7.53 (m, 2H), 7.39 (dd, J = 7.9, 5.6 Hz, 2H), 7.29 (dd, J = 9.3, 2.9 Hz, 1H), 7.18-7.12 (m, 2H), 7.11- 7.06 (m, 2H), 7.01 (d, J = 1.6 Hz, 1H), 6.95 (dd, J = 7.7, 1.6 Hz, 1H), 5.68 (s, 1H), 5.64 (d, J = 6.4 Hz, 1H), 4.59 (d, J = 9.1 Hz, 1H), 4.51 (t, J = 8.2 Hz, 1H), 4.34 (s, 1H), 4.29 (d, J = 4.9 Hz, 2H), 4.18-4.12 (m, 2H), 3.80 (t, J = 5.4 Hz, 2H), 3.72 (dd, J = 5.8, 3.3 Hz, 2H), 3.69 (s, 3H), 3.65-3.60 (m, 2H), 3.57 (dd, J = 6.2, 3.5 Hz, 2H), 3.52 (d, J = 9.9 Hz, 8H), 2.44 (s, 3H), 2.08 (dd, J = 12.8, 7.8 Hz, 1H), 1.91 (ddd, J = 13.0, 8.9, 4.3 Hz, 1H), 1.57 (d, J = 6.3 Hz, 3H), 1.40-1.32 (m, 2H), 1.24-1.21 (m, 2H), 0.95 (s, 9H). |
| 1041 | 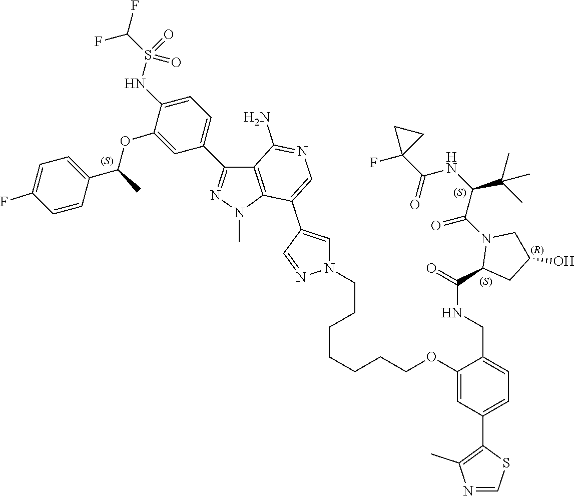 | (2S,4R)-N-[(2-{[9-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}-1H-pyrazol-1- yl)nonyl]oxy}-4-(4-methyl-1,3-thiazol-5-yl)phenyl)methyl]-1-[(2S)-2-[(1- fluorocyclopropyl)formamido]-3,3-dimethylbutanoyl]-4-hydroxypyrrolidine-2-carboxamide From 9-bromononan-1-ol LCMS (Method A1) 3.19 min, m/z: 1215.4 [M + H]+. 1H NMR (400 MHz, CDCI3): 8.66 (s, 1H), 7.68-7.61 (m, 3H), 7.55 (d, J = 3.4 Hz, 2H), 7.48 (s, 1H), 7.39-7.27 (m, 7H), 7.18 (dd, J = 8.2, 1.8 Hz, 1H), 7.04 (d, J = 1.8 Hz, 1H), 6.98 (t, J = 8.4 Hz, 2H), 6.81 (d, J = 8.4 Hz, 2H), 6.73 (d, J = 8.8 Hz, 1H), 6.34 (t, J = 53.7 Hz, 1H), 5.43 (q, J = 6.5 Hz, 1H), 5.01 (s, 1H), 4.71 (t, J = 9.0 Hz, 2H), 4.54 (dd, J = 14.9, 5.6 Hz, 4H), 4.43 (t, J = 4.9 Hz, 2H), 4.32 (dd, J = 14.9, 5.2 Hz, 1H), 4.13 (d, J = 11.4 Hz, 1H), 3.65 (s, 3H), 3.47 (s, 2H), 2.49 (s, 4H), 1.64 (d, J = 6.3 Hz, 3H), 0.99 (s, 9H). |

### Compound 1030: (2S,4R)—N-[(2-{[7-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)heptyl]oxy}-4-(4-methyl-1,3-thiazol-5-yl)phenyl)methyl]-1-[(2R)-2-[(1-fluorocyclopropyl)formamido]-3,3-dimethylbutanoyl]-4-hydroxypyrrolidine-2-carboxamide

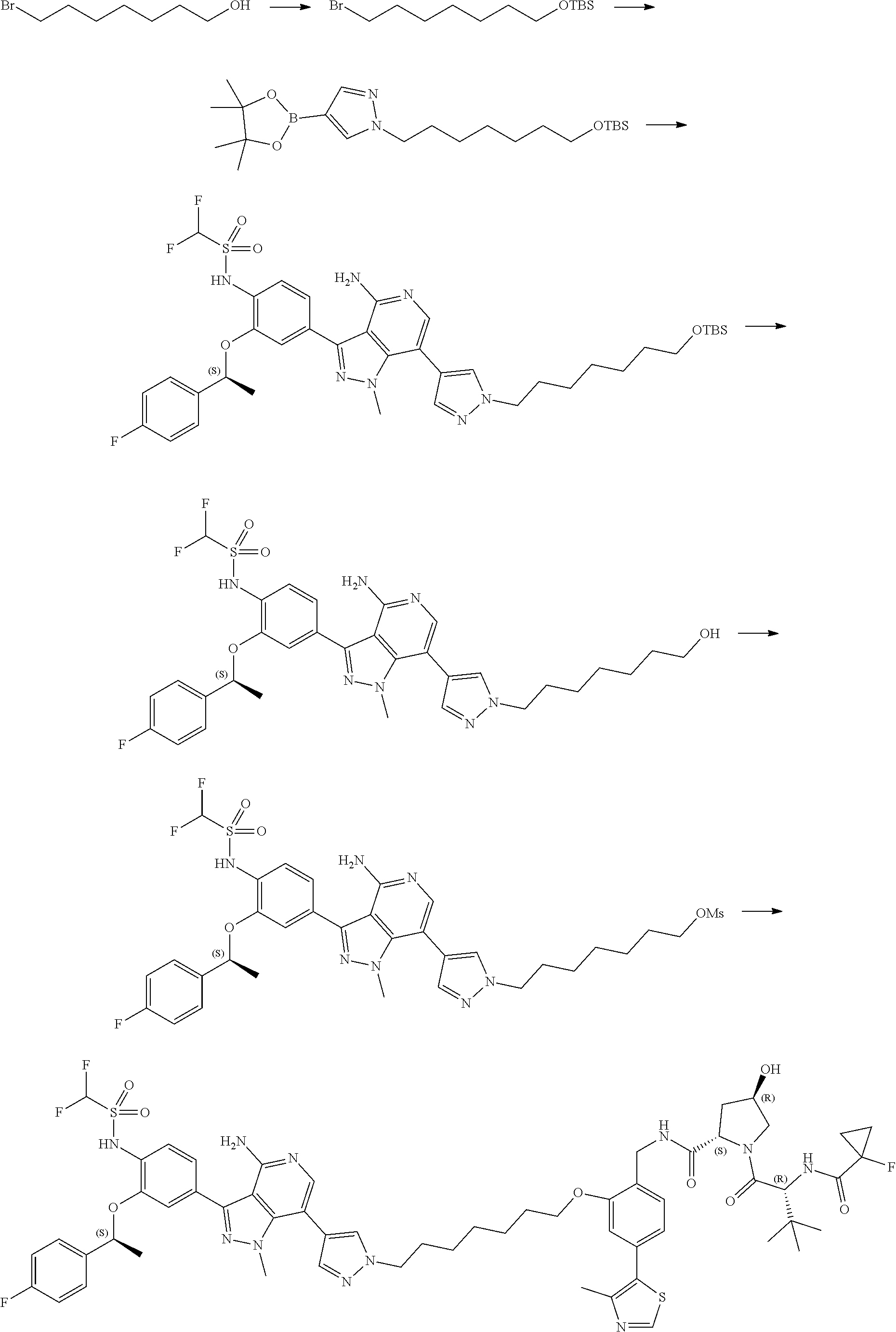

### Step 1: [(7-bromoheptyl)oxy](tert-butyl)dimethylsilane

To a 0° C. solution of 7-bromoheptan-1-ol (1.95 g, 10.0 mmol) in DCM (30 mL) were added TBSCI (1.65 g, 11.0 mmol), Et₃N (1.73 mL, 12.5 mmol) and DMAP (122 mg, 1 mmol). The mixture was allowed to warm to RT and stirred overnight. The mixture was concentrated, and the residue was partitioned between Et₂O (25 ml) and water (25 mL). The layers were separated, and the aqueous phase was extracted with Et₂O (3×25 mL). The combined organics were dried (MgSO₄) and concentrated. The residue was purified by flash chromatography (0-10% DCM:heptane) to afford the title product (2.45 g, 79%) as a colourless oil. ¹H NMR (300 MHz, CDCl₃): 3.60 (t, J=6.5 Hz, 2H), 3.40 (t, J=6.8 Hz, 2H), 1.86 (p, J=6.8 Hz, 2H), 1.55-1.48 (m, 4H), 1.46-1.29 (m, 4H), 0.89 (s, 9H).

### Step 2: 1-{7-[(tert-butyldimethylsilyl)oxy]heptyl}-4-(4,4,5,5-tetramethyl-1,3,2-dioxaborolan-2-yl)-1H-pyrazole

A mixture of 4-(4,4,5,5-tetramethyl-1,3,2-dioxaborolan-2-yl)-1H-pyrazole (500 mg, 2.57 mmol), [(7-bromoheptyl)oxy](tert-butyl)dimethylsilane (1.59 g, 5.14 mmol) and Cs₂CO₃ (3.32 g, 10.2 mmol) in MeCN (12.8 ml) was heated at 80° C. overnight. Once cooled, water was added and the mixture was extracted with EtOAc. The organic phase was washed twice with water and once with brine, dried (MgSO₄) and concentrated. The residue was purified by flash chromatography (0-20% EtOAc:heptane) to afford the title product (656 mg, 61%) as a colourless oil. LCMS (method F): 3.64 min, m/z: 423.0 [M+H]⁺.

### Step 3: N-{4-[4-amino-7-(1-{7-[(tert-butyldimethylsilyl)oxy]heptyl}-1H-pyrazol-4-yl)-1-methyl-1H-pyrazolo[4,3-c]pyridin-3-yl]-2-[(1 S)-1-(4-fluorophenyl)ethoxy]phenyl}-1,1-difluoromethanesulfonamide

To a mixture of N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (200 mg, 0.3239 mmol), 1-{7-[(tert-butyldimethylsilyl)oxy]heptyl}-4-(4,4,5,5-tetramethyl-1,3,2-dioxaborolan-2-yl)-1H-pyrazole (150 mg, 356 μmol) and Na₂CO₃ (68.5 mg, 647 μmol) in DME (5 mL) and water (1 mL) was added Pd(PPh₃)₄ (37.3 mg, 32.3 μmol), and the reaction was heated at 80° C. overnight. Once cooled, the mixture was concentrated and the residue was partitioned between EtOAc and water. The layers were separated, and the aqueous phase was extracted twice with EtOAc. The combined organics were dried (MgSO₄) and concentrated. The residue was purified by flash chromatography (0-5% MeOH:DCM) to afford the title product (126 mg, 50%) as a brown gum. LCMS (method F): 2.85 min, m/z: 786.8 [M+H]⁺.

### Step 4: N-(4-{4-amino-7-[1-(7-hydroxyheptyl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

To a 0° C. solution of N-{4-[4-amino-7-(1-{7-[(tert-butyldimethylsilyl)oxy]heptyl}-1H-pyrazol-4-yl)-1-methyl-1H-pyrazolo[4,3-c]pyridin-3-yl]-2-[(1S)-1-(4-fluorophenyl)ethoxy]phenyl}-1,1-difluoromethanesulfonamide (125 mg, 159 μmol) in THE (5 mL) was added TBAF (190 μL, 190 μmol), and the reaction was stirred at RT overnight. The mixture was concentrated and the residue was purified by reverse-phase chromatography (0-50% H₂O:MeCN, with 0.1% TFA) to afford the title product (67.1 mg, 63%) as a white solid. LCMS (method C): 1.82 min, m/z: 670.0 [M−H]⁻.

### Step 5: 7-(4-{4-amino-3-[4-(difluoromethanesulfonamido)-3-[(1 S)-1-(4-fluorophenyl)ethoxy]phenyl]-1-methyl-1H-pyrazolo[4,3-c]pyridin-7-yl}-1H-pyrazol-1-yl)heptyl methanesulfonate

To a 0° C. solution of N-(4-{4-amino-7-[1-(7-hydroxyheptyl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide (160 mg, 238 μmol) in DCM (5 mL) were added Et₃N (64.8 μL, 476 μmol) and MsCl (18.3 μL, 238 μmol), and the reaction was stirred at RT overnight. The mixture was concentrated and the residue was purified by flash chromatography (0-5% MeOH:DCM) to afford the title product (54.1 mg, 30%) as a brown oil. LCMS (method C): 2.02 min, m/z: 750.2 [M+H]⁺.

### Step 6: (2S,4R)—N-[(2-{[7-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)heptyl]oxy}-4-(4-methyl-1,3-thiazol-5-yl)phenyl)methyl]-1-[(2R)-2-[(1-fluorocyclopropyl)formamido]-3,3-dimethylbutanoyl]-4-hydroxypyrrolidine-2-carboxamide

A mixture of 7-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)heptyl methanesulfonate (45 mg, 60.0 μmol), (2S,4R)-1-[(2R)-2-[(1-fluorocyclopropyl)formamido]-3,3-dimethylbutanoyl]-4-hydroxy-N-{[2-hydroxy-4-(4-methyl-1,3-thiazol-5-yl)phenyl]methyl}pyrrolidine-2-carboxamide (21.3 mg, 40.0 μmol) and K₂CO₃ (11.0 mg, 80.0 μmol) in DMF (1 mL) was heated at 70° C. overnight. Once cooled, the mixture was concentrated and the residue was purified by prep-HPLC to afford the formate salt of the title compound (9.90 mg, 21%) as an off-white solid. LCMS (method G): 2.28 min, m/z 1187.2 [M+H]⁺. ¹H NMR (300 MHz, DMSO-d₆): 8.97 (s, 1H), 8.49 (t, J=5.9 Hz, 1H), 7.92 (d, J=0.8 Hz, 1H), 7.62-7.48 (m, 4H), 7.40 (d, J=7.9 Hz, 2H), 7.33-7.23 (m, 1H), 7.22-7.12 (m, 4H), 7.10 (d, J=1.9 Hz, 1H), 7.00 (d, J=1.7 Hz, 1H), 6.94 (dd, J=7.8, 1.5 Hz, 1H), 5.80 (s, 1H), 5.71 (s, 1H), 5.65 (q, J=6.3 Hz, 1H), 5.16 (s, 1H), 4.59 (d, J=9.2 Hz, 1H), 4.51 (t, J=8.1 Hz, 1H), 4.34 (s, 1H), 4.25 (dd, J=15.6, 5.7 Hz, 2H), 4.16 (t, J=6.7 Hz, 2H), 4.09-4.01 (m, 2H), 3.69 (s, 3H), 3.67 (t, J=6.3 Hz, 1H), 3.61 (s, 2H), 2.45 (s, 3H), 2.06 (d, J=8.5 Hz, 1H), 1.89-1.79 (m, 2H), 1.78-1.72 (m, 2H), 1.58 (d, J=6.3 Hz, 4H), 1.43-1.38 (m, 4H), 1.35-1.27 (m, 4H), 1.26-1.17 (m, 2H), 0.94 (s, 9H).

### Compound 1031: N-(4-{4-amino-7-[1-(7-{[2-(2,6-dioxopiperidin-3-yl)-1,3-dioxo-2,3-dihydro-1H-isoindol-4-yl]oxy}heptyl)-1H-pyrazol-4-yl]-1-methyl-1H-pyrazolo[4,3-c]pyridin-3-yl}-2-[(1 S)-1-(4-fluorophenyl)ethoxy]phenyl)-1,1-difluoromethanesulfonamide

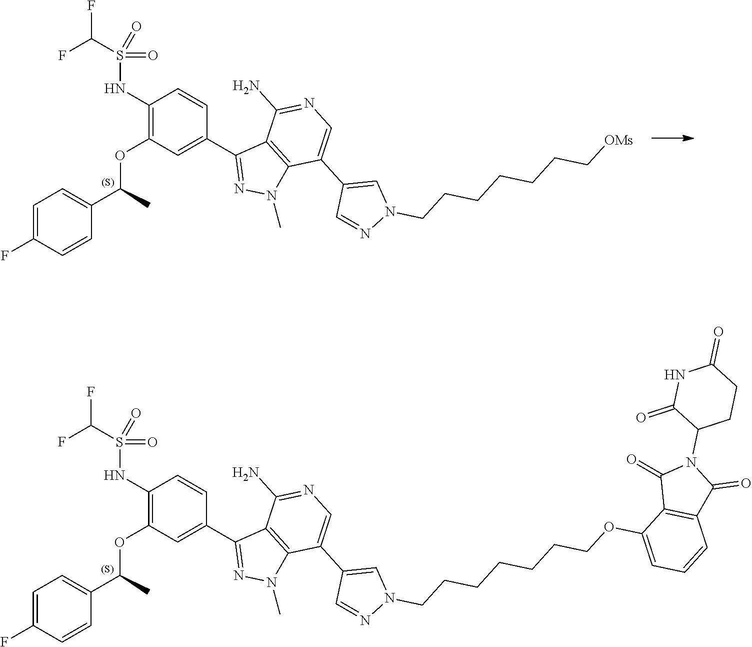

### Step 1: N-(4-{4-amino-7-[1-(7-{[2-(2,6-dioxopiperidin-3-yl)-1,3-dioxo-2,3-dihydro-1H-isoindol-4-yl]oxy}heptyl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

A mixture of 7-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)heptyl methanesulfonate (45 mg, 60.0 μmol), 2-(2,6-dioxopiperidin-3-yl)-4-hydroxy-2,3-dihydro-1H-isoindole-1,3-dione (10.9 mg, 40.0 μmol) and K₂CO₃ (11.0 mg, 80.0 μmol) in DMF (1 mL) was heated at 70° C. overnight. Once cooled, the mixture was concentrated and the residue was purified by prep-HPLC to afford the title compound (3.90 mg, 11%) as a yellow solid. LCMS (method F): 2.16 min, m/z: 926.1 [M−H]⁻. ¹H NMR (300 MHz, DMSO-d₆): 11.12 (s, 1H), 7.92 (s, 1H), 7.81-7.76 (m, 1H), 7.57-7.48 (m, 5H), 7.44 (dd, J=7.2 Hz, 2H), 7.19-7.09 (m, 4H), 6.96 (t, J=52.9 Hz, 1H), 5.70 (bs, 2H), 5.65 (q, J=6.3 Hz, 1H), 5.10-5.05 (m, 1H), 4.20-4.15 (m, 4H), 3.69 (d, J=6.3 Hz, 3H), 2.01-1.98 (m, 1H), 1.83-1.78 (m, 4H), 1.59-1.55 (m, 4H), 1.41-1.23 (m, 5H).

### Compound 1100: N-(4-{4-amino-1-methyl-7-[1-(7-{[2-(1-methyl-2,6-dioxopiperidin-3-yl)-1,3-dioxo-2,3-dihydro-1H-isoindol-4-yl]oxy}heptyl)-1H-pyrazol-4-yl]-1H-pyrazolo[4,3-c]pyridin-3-yl}-2-[(1S)-1-(4-fluorophenyl)ethoxy]phenyl)-1,1-difluoromethanesulfonamide

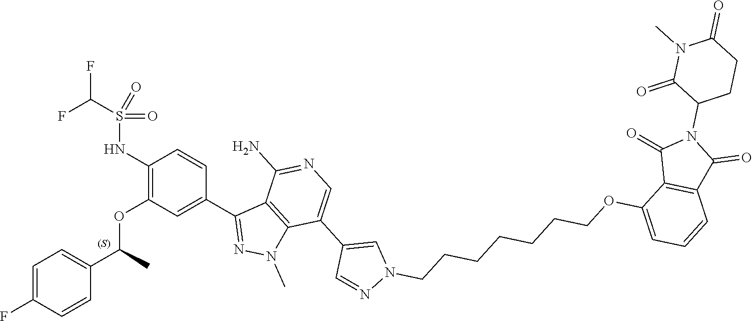

The title compound was prepared according to the procedure for Compound 1031, utilising Intermediate H. LCMS (Method A): 3.12 min, m/z: 942.3 [M+H]⁺. ¹H NMR (400 MHz, DMSO-d₆): 10.62 (s, 1H), 7.92 (s, 1H), 7.80 (dd, J=8.5, 7.3 Hz, 1H), 7.61-7.49 (m, 5H), 7.42 (dd, J=10.1, 7.6 Hz, 2H), 7.22-7.09 (m, 4H), 6.93 (d, J=52.7 Hz, 1H), 5.75 (d, J=7.8 Hz, 2H), 5.65 (q, J=6.3 Hz, 1H), 5.13 (dd, J=13.0, 5.4 Hz, 1H), 4.18 (dt, J=17.1, 6.6 Hz, 5H), 3.69 (s, 3H), 3.00 (s, 3H), 2.91 (ddd, J=13.8, 9.7, 6.8 Hz, 2H), 2.77-2.66 (m, 2H), 2.01 (td, J=8.3, 4.1 Hz, 2H), 1.80 (dp, J=35.5, 6.9 Hz, 5H), 1.58 (d, J=6.2 Hz, 3H), 1.43 (dp, J=22.0, 7.4 Hz, 6H).

### Compound 1060: N-(4-{4-amino-7-[1-(7-{[2-(2,6-dioxopiperidin-3-yl)-1,3-dioxo-2,3-dihydro-1H-isoindol-4-yl]oxy}heptyl)-1H-pyrazol-4-yl]-1-methyl-1H-pyrazolo[4,3-c]pyridin-3-yl}-2-fluorophenyl)-1,1-difluoromethanesulfonamide

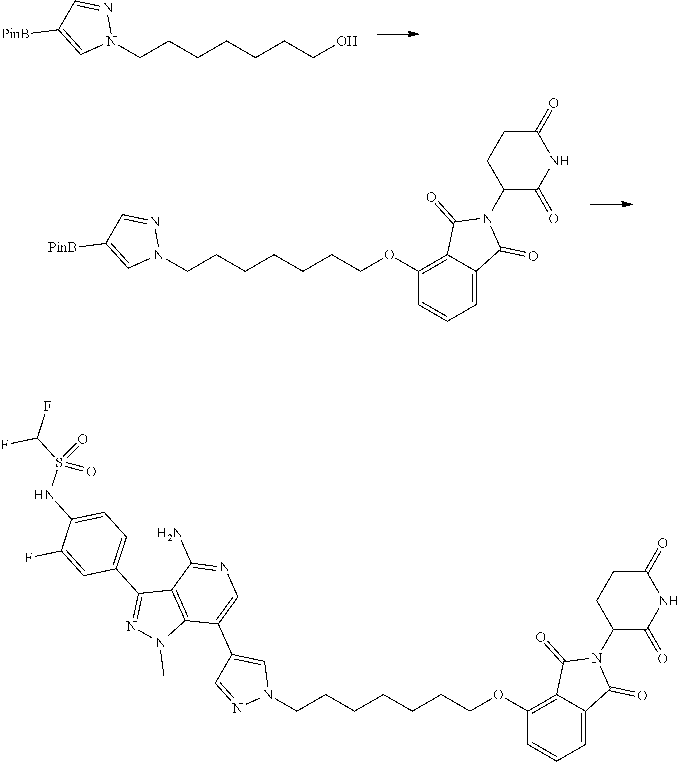

### Step 1: 2-(2,6-dioxopiperidin-3-yl)-4-({7-[4-(4,4,5,5-tetramethyl-1,3,2-dioxaborolan-2-yl)-1H-pyrazol-1-yl]heptyl}oxy)-2,3-dihydro-1H-isoindole-1,3-dione

A mixture of 2-(2,6-dioxopiperidin-3-yl)-4-hydroxy-2,3-dihydro-1H-isoindole-1,3-dione (100 mg, 364 μmol), 7-[4-(4,4,5,5-tetramethyl-1,3,2-dioxaborolan-2-yl)-1H-pyrazol-1-yl]heptan-1-ol (112 mg, 364 μmol), PPh₃ (285 mg, 1.09 mmol) and DIAD (14.7 mg, 72.8 μmol) in THE (3 mL) was stirred at 0° C. under N₂ for 3 h. The mixture was concentrated, and the residue was poured into water and extracted with EtOAc. The combined organic phases were dried over Na₂SO₄ and concentrated. The residue was purified by Prep-TLC (DCM/MeOH=20/1) to afford the title compound (100 mg, 49%) as a yellow solid. LCMS (Method A): 4.25 min, m/z: 565.2 [M+H]⁺.

### Step 2: N-(4-{4-amino-7-[1-(7-{[2-(2,6-dioxopiperidin-3-yl)-1,3-dioxo-2,3-dihydro-1H-isoindol-4-yl]oxy}heptyl)-1H-pyrazol-4-yl]-1-methyl-1H-pyrazolo[4,3-c]pyridin-3-yl}-2-fluorophenyl)-1,1-difluoromethanesulfonamide

A mixture of 2-(2,6-dioxopiperidin-3-yl)-4-({7-[4-(4,4,5,5-tetramethyl-1,3,2-dioxaborolan-2-yl)-1H-pyrazol-1-yl]heptyl}oxy)-2,3-dihydro-1H-isoindole-1,3-dione (100 mg, 177 μmol), N-(4-{4-amino-7-iodo-1-methyl-1H-pyrazolo[4,3-c]pyridin-3-yl}-2-fluorophenyl)-1,1-difluoromethanesulfonamide (88.0 mg, 177 μmol), Pd(dppf)Cl₂ (12.9 mg, 17.7 μmol) and Na₂CO₃ (56.2 mg, 531 μmol) in 80% aq. dioxane (3 mL) was stirred at 100° C. under N₂ overnight. The mixture was concentrated, and the residue was poured into water and extracted with EtOAc. The combined organic phases were dried over Na₂SO₄ and concentrated. The residue was purified by Prep-HPLC to afford the title compound (10 mg, 7%) as a yellow solid. LCMS (acidic 5 min): 4.25 min, m/z: 808.7 [M+H]⁺. ¹H NMR (400 MHz, DMSO-d₆): 13.26 (s, 1H), 11.23 (s, 1H), 11.09 (s, 1H), 8.09 (s, 1H), 7.96 (s, 2H), 7.80 (dd, J=8.5, 7.3 Hz, 1H), 7.71 (s, 1H), 7.66 (s, 1H), 7.62-7.58 (m, 1H), 7.56 (d, J=8.1 Hz, 1H), 7.50 (d, J=2.2 Hz, 1H), 7.44 (s, 1H), 5.07 (dd, J=12.9, 5.3 Hz, 1H), 4.20 (td, J=6.7, 2.8 Hz, 4H), 3.79 (s, 3H), 2.54 (s, 2H), 2.00 (dt, J=14.7, 4.9 Hz, 2H), 1.84 (q, J=7.2 Hz, 2H), 1.74 (d, J=7.5 Hz, 2H), 1.49-1.37 (m, 4H), 1.29 (q, J=8.9, 8.4 Hz, 4H).

### Compound 1059: N-(4-{4-amino-7-[1-(7-{[2-(2,6-dioxopiperidin-3-yl)-1,3-dioxo-2,3-dihydro-1H-isoindol-4-yl]oxy}heptyl)-1H-pyrazol-4-yl]-1-methyl-1H-pyrazolo[4,3-c]pyridin-3-yl}-2-fluorophenyl)ethane-1-sulfonamide

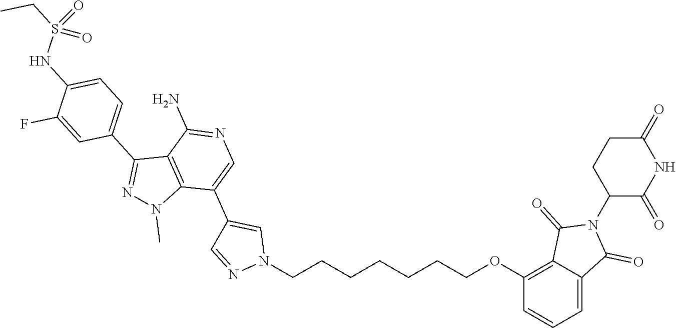 [SMILES: CCS(=O)(=O)Nc1ccc(-c2nn(C)c3c(-c4cnn(CCCCCCCOc5cccc6c5C(=O)N(C5CCC(=O)NC5=O)C6=O)c4)cnc(N)c23)cc1F]

The title compound was prepared according to the procedure for the synthesis of Compound 1060, using Intermediate A1B20 in Step 2. LCMS (Method A): 3.42 min, m/z: 786.7 [M+H]⁺. ¹H NMR (400 MHz, DMSO-d₆): 11.13 (s, 1H), 9.83 (s, 1H), 7.93 (s, 1H), 7.79 (dd, J=8.5, 7.3 Hz, 1H), 7.60-7.40 (m, 7H), 5.86 (s, 2H), 5.06 (dd, J=12.9, 5.4 Hz, 1H), 4.18 (dt, J=13.6, 6.6 Hz, 4H), 3.73 (s, 3H), 3.18 (q, J=7.3 Hz, 2H), 2.92-2.78 (m, 1H), 2.60-2.53 (m, 2H), 2.04-1.94 (m, 1H), 1.84 (p, J=7.0 Hz, 2H), 1.74 (q, J=6.8 Hz, 2H), 1.49-1.41 (m, 2H), 1.39 (d, J=7.4 Hz, 2H), 1.30 (d, J=7.3 Hz, 3H), 1.24 (d, J=8.8 Hz, 2H), 0.92-0.77 (m, 1H).

### Intermediate H: 4-hydroxy-2-(1-methyl-2,6-dioxopiperidin-3-yl)-2,3-dihydro-1H-isoindole-1,3-dione

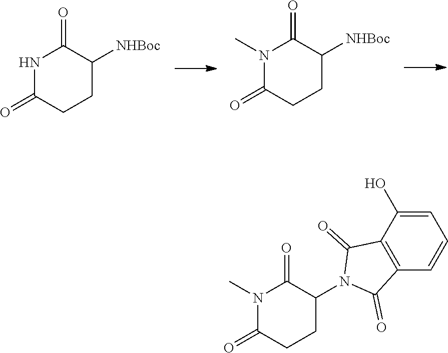 [SMILES: CC(C)(C)OC(=O)NC1CCC(=O)NC1=O ; CN1C(=O)CCC(NC(=O)OC(C)(C)C)C1=O ; CN1C(=O)CCC(N2C(=O)c3cccc(O)c3C2=O)C1=O]

### Step 1: tert-butyl N-(1-methyl-2,6-dioxopiperidin-3-yl)carbamate

To a solution of tert-butyl N-(2,6-dioxopiperidin-3-yl)carbamate (4.0 g, 17.5 mmol) in DMF (40 mL) were added Mel (2.0 g, 14.0 mmol) and K₂CO₃ (7.25 g, 52.5 mmol), and the mixture was stirred at 50° C. under N₂ for 8 h. The mixture was filtered, and the filtrate was concentrated under reduced pressure. The residue was purified by column chromatography (PE:EtOAc, 5:1) to afford the title compound (401 mg, 9%) as a white solid. LCMS (Method A): 2.51 min, m/z: 265.1 [M+H]⁺.

### Step 2: 3-amino-1-methylpiperidine-2,6-dione

A solution of tert-butyl N-(1-methyl-2,6-dioxopiperidin-3-yl)carbamate (840 mg, 3.46 mmol) in 4M HCl/1,4-dioxane (5 mL) was stirred at RT for 2 h. The mixture was concentrated to afford the title compound (660 mg, >100%) as a yellow solid. LCMS (Method A): 0.30 min, m/z: 143.2 [M+H]⁺.

### Step 3: 4-hydroxy-2-(1-methyl-2,6-dioxopiperidin-3-yl)-2,3-dihydro-1H-isoindole-1,3-dione

A mixture of 3-amino-1-methylpiperidine-2,6-dione (300 mg, 2.11 mmol), 3-hydroxybenzene-1,2-dicarboxylic acid (385 mg, 2.11 mmol) and AcONa (520 mg, 6.34 mmol) in AcOH (4 mL) was stirred at 120° C. under N₂ for 12 h. The mixture was concentrated and the residue was purified by prep-TLC (PE:EtOAc, 1:1) to afford the title compound (41.0 mg, 7%) as a yellow solid. LCMS (Method A): 2.44 min, m/z: 289.1 [M+H]⁺.

### Compound 1062: 7-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)-N-[2-(2,6-dioxopiperidin-3-yl)-1-oxo-2,3-dihydro-1H-isoindol-4-yl]heptanamide

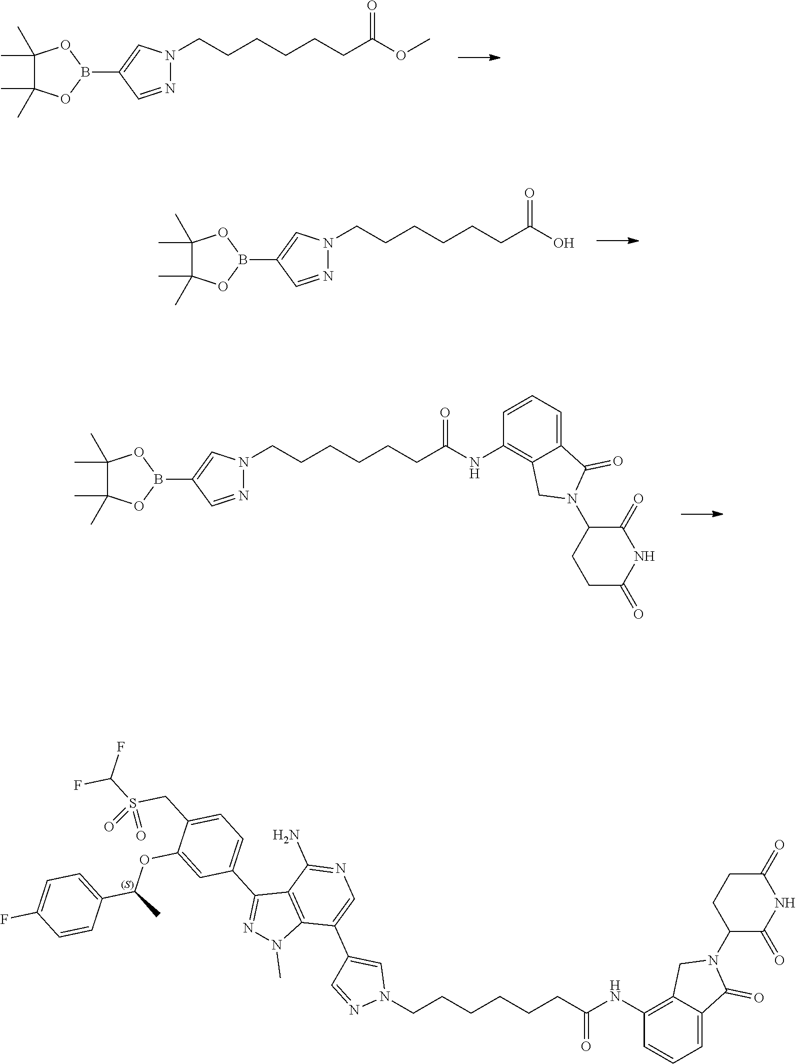

### Step 1: 7-[4-(4,4,5,5-tetramethyl-1,3,2-dioxaborolan-2-yl)-1H-pyrazol-1-yl]heptanoic acid

A mixture of methyl 7-[4-(4,4,5,5-tetramethyl-1,3,2-dioxaborolan-2-yl)-1H-pyrazol-1-yl]heptanoate (1.5 g, 4.46 mmol) and NaOH (891 mg, 22.3 mmol) in 80% aq. MeOH (12 mL) was stirred at 60° C. for 4 h. The mixture was filtered through Celite and the filter cake was washed with MeOH. The filtrate was concentrated under reduced pressure and the crude residue was purified by column chromatography (PE:EtOAc, 3:1) to afford the title product (600 mg, 42%) as a yellow oil. LCMS (Method A): 3.64 min; m/z: 323.2 [M+H]⁺.

### Step 2: N-[2-(2,6-dioxopiperidin-3-yl)-1-oxo-2,3-dihydro-1H-isoindol-4-yl]-7-[4-(4,4,5,5-tetramethyl-1,3,2-dioxaborolan-2-yl)-1H-pyrazol-1-yl]heptanamide

A mixture of 7-[4-(4,4,5,5-tetramethyl-1,3,2-dioxaborolan-2-yl)-1H-pyrazol-1-yl]heptanoic acid (600 mg, 1.86 mmol), 3-(4-amino-1-oxo-2,3-dihydro-1H-isoindol-2-yl)piperidine-2,6-dione (528 mg, 2.04 mmol), EDCI (427 mg, 2.23 mmol) and DMAP (272 mg, 2.23 mmol) in DMF (6 mL) was stirred under N₂ at RT for 4 h. The mixture was filtered through Celite and the filter cake was washed with MeOH. The filtrate was concentrated under reduced pressure and the residue was purified by column chromatography (PE:EtOAc, 3:1) to afford the title product (472 mg, 45%) as a yellow oil. LCMS (Method A): 3.70 min; m/z: 564.5 [M+H]⁺.

### Step 3: 7-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)-N-[2-(2,6-dioxopiperidin-3-yl)-1-oxo-2,3-dihydro-1H-isoindol-4-yl]heptanamide

A mixture of N-[2-(2,6-dioxopiperidin-3-yl)-1-oxo-2,3-dihydro-1H-isoindol-4-yl]-7-[4-(4,4,5,5-tetramethyl-1,3,2-dioxaborolan-2-yl)-1H-pyrazol-1-yl]heptanamide (350 mg, 621 μmol), N-(4-{4-amino-7-iodo-1-methyl-1H-pyrazolo[4,3-c]pyridin-3-yl}-2-[(1 S)-1-(4-fluorophenyl)ethoxy]phenyl)-1,1-difluoromethanesulfonamide (344 mg, 558 μmol), Pd(dppfCl₂ (50.3 mg, 62.1 μmol) and K₃PO₄ (658 mg, 3.10 mmol) in DMSO (6 mL) was stirred under N₂ at 90° C. for 16 h. Once cooled, the mixture was filtered over Celite and the filter cake was washed with MeOH. The filtrate was concentrated under reduced pressure, and the residue was purified by Prep-HPLC to afford the title product (200 mg, 35%) as a yellow solid. LCMS (Method A): 2.78 min; m/z: 927 [M+H]⁺. ¹H NMR (400 MHz, DMSO-d₆): 11.02 (s, 1H), 10.60 (s, 1H), 9.79 (s, 1H), 8.06 (s, 1H), 7.81 (d, J=7.1 Hz, 1H), 7.69 (s, 1H), 7.64-7.53 (m, 4H), 7.52-7.42 (m, 3H), 7.22-7.11 (m, 4H), 6.97 (d, J=52.6 Hz, 1H), 5.65 (d, J=6.4 Hz, 1H), 5.14 (dd, J=13.3, 5.0 Hz, 1H), 4.44-4.29 (m, 2H), 4.19 (t, J=6.9 Hz, 2H), 3.76 (s, 3H), 2.91 (ddd, J=17.8, 13.6, 5.3 Hz, 1H), 2.57 (s, 1H), 2.36 (t, J=7.4 Hz, 2H), 2.11-1.93 (m, 2H), 1.85 (t, J=7.1 Hz, 2H), 1.62 (s, 1H), 1.59 (d, J=6.5 Hz, 3H), 1.42-1.33 (m, 2H), 1.30 (d, J=6.9 Hz, 2H), 1.22-1.11 (m, 1H), 0.89-0.81 (m, 1H).

### Intermediate I: tert-butyl N-[(1S)-1-{[(2S)-1-[(2S,4S)-4-amino-2-[(2,6-difluorophenyl) carbamoyl]pyrrolidin-1-yl]-3,3-dimethyl-1-oxobutan-2-yl]carbamoyl}ethyl]-N-methylcarbamate

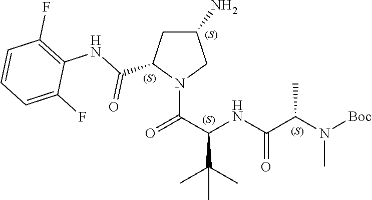

Intermediate I was prepared according to the procedures reported in Mares, A., Miah, A. H., Smith, I. E. D. et al. Commun. Biol. 3,140 (2020). LCMS (Method G): 1.55 min, m/z: 540.2 [M+H]⁺.

### Compound 1089: (2S)-4-{2-[2-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)ethoxy]acetamido}-N-(2,6-difluorophenyl)-1-[(2S)-3,3-dimethyl-2-[(2S)-2-(methylamino)propanamido]butanoyl]pyrrolidine-2-carboxamide

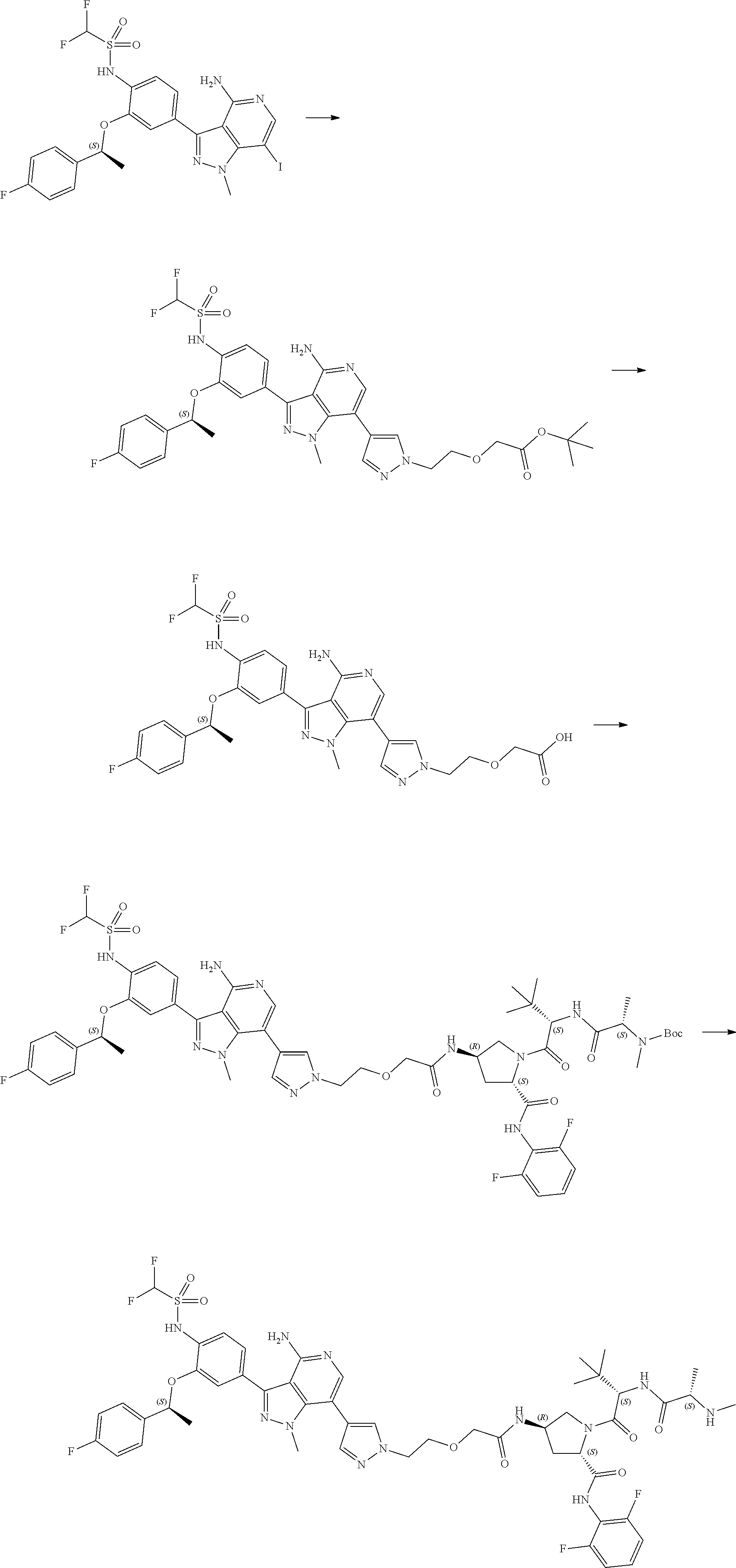

### Step 2: 2-[2-(4-{4-amino-3-[4-(difluoromethanesulfonamido)-3-[(1S)-1-(4-fluorophenyl)ethoxy]phenyl]-1-methyl-1H-pyrazolo[43-c]pyridin-7-yl}-1H-pyrazol-1-yl}ethoxy]acetic acid

A solution of tert-butyl 2-[2-(4-{4-amino-3-[4-(difluoromethanesulfonamido)-3-[(1S)-1-(4-fluoro phenyl)ethoxy]phenyl]-1-methyl-1H-pyrazolo[4,3-c]pyridin-7-yl}-1H-pyrazol-1-yl)ethoxy]acetate (500 mg, 698 μmol) in HCOOH (5 mL) was stirred at RT for 1 h. The mixture was concentrated, and the residue was purified by column chromatography (DCM/MeOH=30/1) to afford the title compound (300 mg, 65%) as a yellow solid. LCMS (Method A): 2.59 min, m/z: 600.2 [M+H]⁺.

### Step 3: tert-butyl N-[(1S)-1-{[(2S)-1-[(2S)-4-{2-[2-(4-{4-amino-3-[4-(difluoromethanesulfonamido)-3-[(1 S)-1-(4-fluorophenyl)ethoxy]phenyl]-1-methyl-1H-pyrazolo[4,3-c]pyridin-7-yl}-1H-pyrazol-1-yl)ethoxy]acetamido}-2-[(2,6-difluorophenyl)carbamoyl]pyrrolidin-1-yl]-3,3-dimethyl-1-oxobutan-2-yl]carbamoyl}ethyl]-N-methylcarbamate

To a mixture of 2-[2-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)ethoxy]acetic acid (300 mg, 454 μmol), DIPEA (293 mg, 2.27 mmol) and HATU (172 mg, 454 μmol) in DMF (10 mL) was added tert-butyl N-[(1S)-1-{[(2S)-1-[(2S)-4-amino-2-[(2,6-difluorophenyl)carbamoyl]pyrrolidin-1-yl]-3,3-dimethyl-1-oxobutan-2-yl]carbamoyl}ethyl]-N-methylcarbamate (244 mg, 454 μmol), and the mixture was stirred at RT for 2 h. The mixture was concentrated, and the residue was purified by Prep-TLC (DCM/MeOH=20/1) to afford the title compound (90.0 mg, 17%) as a yellow solid. LCMS (Method A): 3.04 min, m/z: 1181.3 [M+H]⁺.

### Step 4: (2S)-4-{2-[2-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)ethoxy]acetamido}-N-(2,6-difluorophenyl)-1-[(2S)-3,3-dimethyl-2-[(2S)-2-(methylamino)propanamido]butanoyl]pyrrolidine-2-carboxamide

A solution of tert-butyl N-[(1S)-1-{[(2S)-1-[(2S)-4-{2-[2-(4-{4-amino-3-[4-(difluoromethane sulfonamido)-3-[(1S)-1-(4-fluorophenyl)ethoxy]phenyl]-1-methyl-1H-pyrazolo [4,3-c]pyridin-7-yl}-1H-pyrazol-1-yl)ethoxy]acetamido}-2-[(2,6-difluorophenyl)carbamoyl]pyrrolidin-1-yl]-3,3-dimethyl-1-oxobutan-2-yl]carbamoyl}ethyl]-N-methylcarbamate (90 mg, 76.1 μmol) in HCO₂H (2 mL) was stirred at rt for 1 h. The mixture was concentrated and neutralized with sat. aq. Na₂CO₃. The residue was purified by Prep-TLC (DCM/MeOH=20/1) to afford the title compound (10.0 mg, 12%) as a yellow solid. LCMS (Method A): 2.42 min, 1081.3 [M+H]⁺. ¹HNMR (400 MHz, DMSO-d₆): 10.10 (s, 1H), 8.68 (s, 1H), 8.35 (d, J=8.8 Hz, 1H), 7.84 (s, 1H), 7.59 (s, 1H), 7.53-7.49 (m, 3H), 7.35 (d, J=8 Hz, 1H), 7.25-7.21 (m, 1H), 7.14-6.89 (m, 6H), 6.46 (t, J=54 Hz, 1H), 5.64 (q, J=6.4 Hz, 3H), 4.62-4.50 (m, 3H), 4.28 (t, J=5.2 Hz, 2H), 4.09-4.05 (m, 1H), 3.88-3.81 (m, 4H), 3.72 (s, 1H), 3.64 (s, 3H), 3.57-3.53 (m, 1H), 2.54-2.52 (s, 3H), 2.29 (s, 2H), 2.03-1.84 (s, 2H), 1.51 (d, J=6.4 Hz, 3H), 1.30 (d, J=6.8 Hz, 3H), 0.95 (s, 9H).

The following examples were prepared according to the procedure for the synthesis of Compound 1089 utilising the indicated Intermediate F in Step 1.

TABLE 16
Compounds 1080 and 1101

| Example | | |
|---|---|---|
| No. | Structure | Compound Name and Analytical Data |
| 1080 | 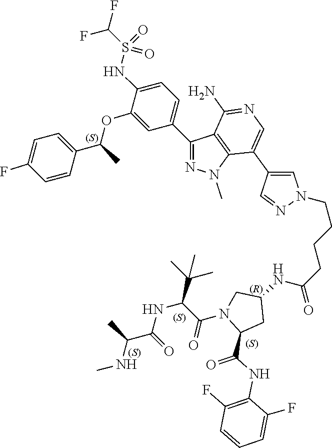 | (2S)-4-[5-(4-{4-amino-3-[4-(difluoromethanesulfonamido)-3-[(1S)-1-(4- fluorophenyl)ethoxy]phenyl]-1-methyl-1H-pyrazolo[4,3-c]pyridin-7-yl}- 1H-pyrazol-1-yl)pentanamido]-N-(2,6-difluorophenyl)-1-[(2S)-3,3- dimethyl-2-[(2S)-2-(methylamino)propanamido]butanoyl]pyrrolidine- 2-carboxamide From Intermediate F2. LCMS (method A): 2.48 min, m/z: 540.4 [M + 2H]²⁺. ¹H NMR (400 MHz, DMSO-d₆): 8.44 (s, 1H), 8.16 (d, J = 7.5 Hz, 1H), 7.92 (s, 1H), 7.57 (s, 1H), 7.50 (td, J = 5.6, 2.6 Hz, 3H), 7.31 (dd, J = 10.4, 8.1 Hz, 2H), 7.23 (t, J = 7.5 Hz, 1H), 7.16-7.08 (m, 3H), 6.92-6.86 (m, 1H), 6.82 (d, J = 2.1 Hz, 1H), 5.65 (q, J = 5.8, 5.2 Hz, 2H), 4.59-4.52 (m, 1H), 4.36-4.26 (m, 1H), 4.16 (t, J = 6.7 Hz, 2H), 3.97 (s, 1H), 3.67 (s, 3H), 2.18 (s, 1H), 2.12 (d, J = 4.8 Hz, 1H), 2.04-1.98 (m, 1H), 1.81 (dd, J = 14.8, 7.5 Hz, 2H), 1.49 (d, J = 6.4 Hz, 3H), 1.35 (s, 1H), 1.24 (s, 9H), 1.08 (d, J = 6.8 Hz, 2H), 0.95 (s, 3H). |
| 1101 | 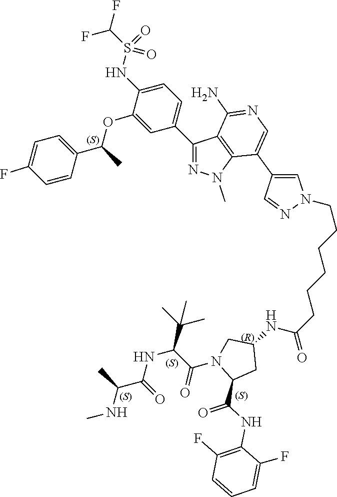 | (2S)-4-[7-(4-{4-amino-3-[4-(difluoromethanesulfonamido)-3-[(1S)-1- (4-fluorophenyl)ethoxy]phenyl]-1-methyl-1H-pyrazolo[4,3-c]pyridin-7- yl}-1H-pyrazol-1-yl)heptanamido]-N-(2,6-difluorophenyl)-1-[(2S)-3,3- dimethyl-2-[(2S)-2-(methylamino)propanamido]butanoyl]pyrrolidine-2- carboxamide From Intermediate F3. LCMS (Method A): 3.35 min, m/z: 554.2 [M + 2H]²⁺. ¹H NMR (400 MHz, DMSO-d₆): 10.04 (s, 1H), 8.88 (d, J = 9.5 Hz, 2H), 8.10 (s, 1H), 7.75 (s, 1H), 7.67 (s, 3H), 7.51 (d, J = 8.0 Hz, 1H), 7.40 (d, J = 7.5 Hz, 1H), 7.22 (d, J = 20.9 Hz, 7H), 6.97 (s, 1H), 5.71 (d, J = 6.4 Hz, 1H), 4.75 (d, J = 9.5 Hz, 1H), 4.64 (d, J = 8.0 Hz, 1H), 4.40 (d, J = 8.0 Hz, 1H), 4.24 (s, 3H), 3.94 (d, J = 6.9 Hz, 1H), 3.81 (s, 3H), 2.61 (d, J = 2.2 Hz, 2H), 2.50 (s, 3H), 2.13 (t, J = 7.3 Hz, 2H), 1.88 (d, J = 7.5 Hz, 2H), 1.66 (d, J = 6.2 Hz, 3H), 1.55 (d, J = 7.7 Hz, 2H), 1.43 (d, J = 6.8 Hz, 3H), 1.31 (s, 7H), 1.01 (s, 9H). |

### Compound 1090: (2S)-4-[9-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)nonanamido]-N-(2,6-difluorophenyl)-1-[(2S)-3,3-dimethyl-2-[(2S)-2-(methylamino)propanamido]butanoyl]pyrrolidine-2-carboxamide

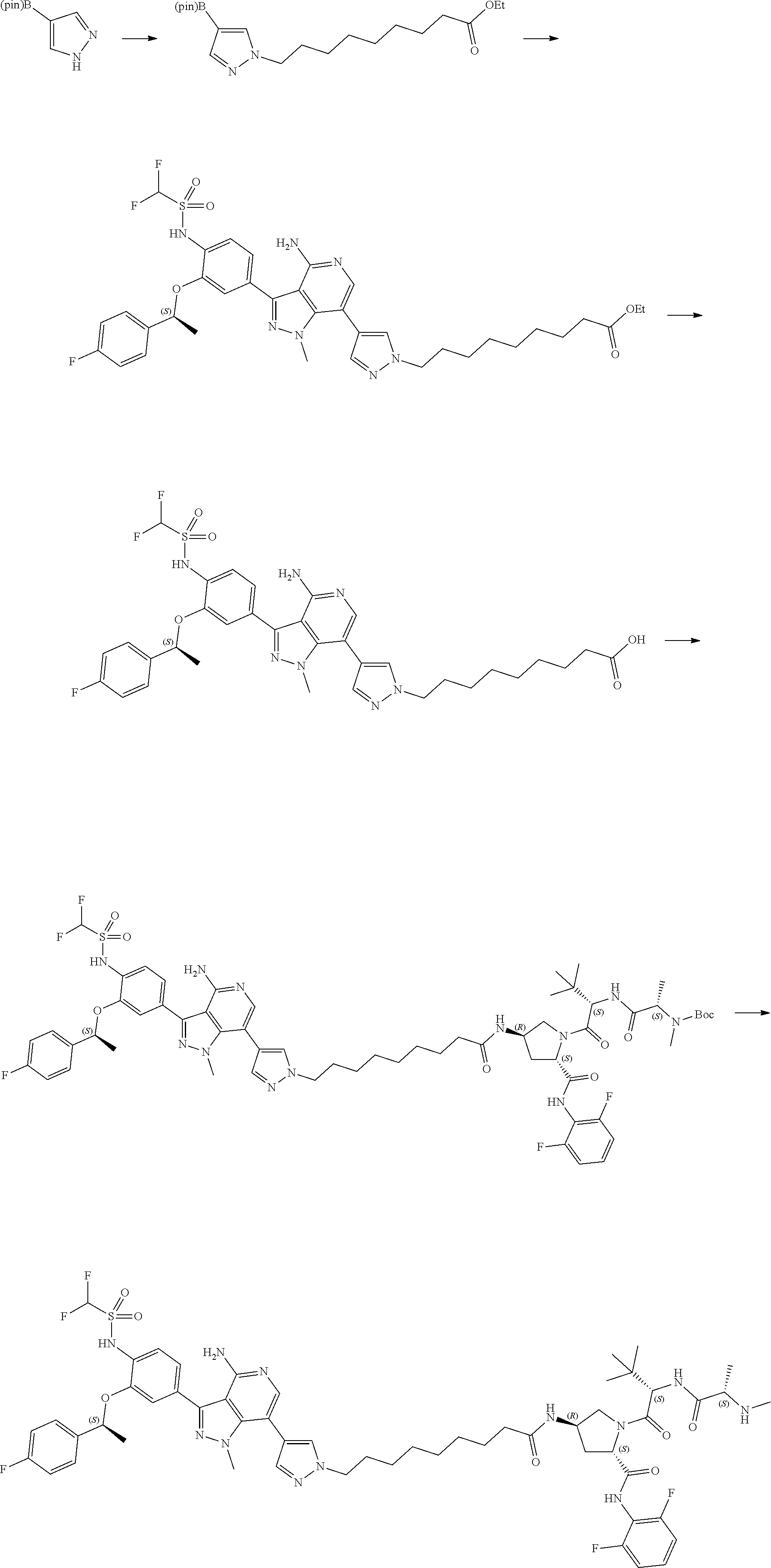

### Step 1: ethyl 9-[4-(4,4,5,5-tetramethyl-1,3,2-dioxaborolan-2-yl)-1H-pyrazol-1-yl]nonanoate

A mixture of ethyl 9-bromononanoate (10.5 g, 5.65 mmol) 4-(4,4,5,5-tetramethyl-1,3,2-dioxaborolan-2-yl)-1H-pyrazole (995 mg, 5.13 mmol) and Cs₂CO₃ (5.01 g, 15.4 mmol) in MeCN (25 mL) was stirred at 80° C. for 16 h. The mixture was concentrated and the residue was purified by column chromatography (DCM/MeOH=200/1) to afford the title product (1.5 g, 90%) as a yellow solid. LCMS (method A): 4.09 min, m/z 379.3 [M+H]⁺.

### Step 2: ethyl 9-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)nonanoate

A mixture of ethyl 9-[4-(4,4,5,5-tetramethyl-1,3,2-dioxaborolan-2-yl)-1H-pyrazol-1-yl]nonanoate (500 mg, 1.32 mmol), N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (570 mg, 924 μmol), Pd(dppf)Cl₂ (48.2 mg, 66.0 μmol) and Na₂CO₃ (419 mg, 3.96 mmol) in Dioxane:water (5:1, 18 mL) was stirred at 80° C. for 16 h. The mixture was concentrated, and the residue was purified by Prep-TLC (DCM/MeOH=30/1) to afford the title compound (400 mg, 90%) as a yellow soild. LCMS (Method A): 3.19 min, m/z: 742.3 [M+H]⁺.

### Step 3: 9-(4-{4-amino-3-[4-(difluoromethanesulfonamido)-3-[(1 S)-1-(4-fluorophenyl)ethoxy]phenyl]-1-methyl-1H-pyrazolo[4,3-c]pyridin-7-yl-}1H-pyrazol-1-yl)nonanoic acid

To a solution of ethyl 9-(4-{4-amino-3-[4-(difluoromethanesulfonamido)-3-[(1 S)-1-(4-fluorophenyl) ethoxy]phenyl]-1-methyl-1H-pyrazolo[4,3-c]pyridin-7-yl}-1H-pyrazol-1-yl)nonanoate (200 mg, 269 μmol) in MeCN (20 mL) was added NaOH (32.2 mg, 807 μmol), and the resulting mixture as stirred at 80° C. for 16 h. The mixture was concentrated, and the residue was purified by Prep-TLC (DCM/MeOH=15/1) to afford the title compound (150 mg, 78%) as a yellow solid. LCMS (Method A): 2.97 min, m/z: 714.3 [M+H]⁺.

### Step 4: tert-butyl N-[(1S)-1-{[(2S)-1-[(2S)-4-[9-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)nonanamido]-2-[(2,6-difluorophenyl)carbamoyl]pyrrolidin-1-yl]-3,3-dimethyl-1-oxobutan-2-yl]carbamoyl}ethyl]-N-methylcarbamate

A mixture of 9-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)nonanoic acid (50 mg, 70.0 μmol), Et₃N (21.2 mg, 210 μmol), HATU (26.7 mg, 70.0 μmol) and tert-butyl N-[(1S)-1-{[(2S)-1-[(2S)-4-amino-2-[(2,6-difluorophenyl)carbamoyl]pyrrolidin-1-yl]-3,3-dimethyl-1-oxobutan-2-yl]carbamoyl}ethyl]-N-methylcarbamate (41.5 mg, 77.0 μmol) in MeCN (5 mL) was stirred at RT for 2 h. The mixture was concentrated, and the residue was purified by Prep-TLC (DCM/MeOH=15/1) to afford the title compound (30 mg, 35%). LCMS (Method A): 2.75 min, m/z: 568.3 [M+2H]²⁺.

### Step 5: (2S)-4-[9-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)nonanamido]-N-(2,6-difluorophenyl)-1-[(2S)-3,3-dimethyl-2-[(2S)-2-(methylamino)propanamido]butanoyl]pyrrolidine-2-carboxamide

A solution of tert-butyl N-[(1S)-1-{[(2S)-1-[(2S)-4-[9-(4-{4-amino-3-[4-(difluoromethane sulfonamido)-3-[(1S)-1-(4-fluorophenyl)ethoxy]phenyl]-1-methyl-1H-pyrazolo[4,3-c]pyridin-7-yl}-1H-pyrazol-1-yl)nonanamido]-2-[(2,6-difluorophenyl)carbamoyl]pyrrolidin-1-yl]-3,3-dimethyl-1-oxobutan-2-yl]carbamoyl}ethyl]-N-methylcarbamate (25 mg, 20.2 μmol) in HCO₂H (3 mL) was stirred at RT for 2 h. The mixture was concentrated and neutralized with sat. aq. Na₂CO. The residue was purified by Prep-HPLC to afford the title compound (6.0 mg, 26%) as a yellow solid. LCMS: (method A): 2.69 min, m/z: 568.4 [M+2H]²⁺. ¹H NMR (400 MHz, MeOD-d₄): 7.81 (s, 1H), 7.61 (s, 1H), 7.50 (d, J=5.4 Hz, 3H), 7.28 (s, 1H), 7.17 (s, 1H), 7.11-6.95 (m, 5H), 6.66 (s, 1H), 5.56 (s, 1H), 5.34 (s, 1H), 4.69-4.63 (m, 2H), 4.22 (d, J=6.8 Hz, 2H), 3.73 (s, 3H), 3.13 (d, J=1.6 Hz, 1H), 3.04 (d, J=7.3 Hz, 1H), 2.67 (d, J=11.5 Hz, 1H), 2.49 (s, 3H), 2.22-2.12 (m, 4H), 2.06-2.00 (m, 2H), 1.90 (s, 2H), 1.69 (d, J=6.2 Hz, 3H), 1.59 (s, 5H), 1.41 (d, J=6.3 Hz, 3H), 1.32 (s, 6H), 1.05 (s, 9H), 0.90 (t, J=6.6 Hz, 3H).

### Compound 1139: (2S)-4-(11-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}undec-10-ynamido)-N-(2,6-difluorophenyl)-1-[(2S)-3,3-dimethyl-2-[(2S)-2-(methylamino)propanamido]butanoyl]pyrrolidine-2-carboxamide

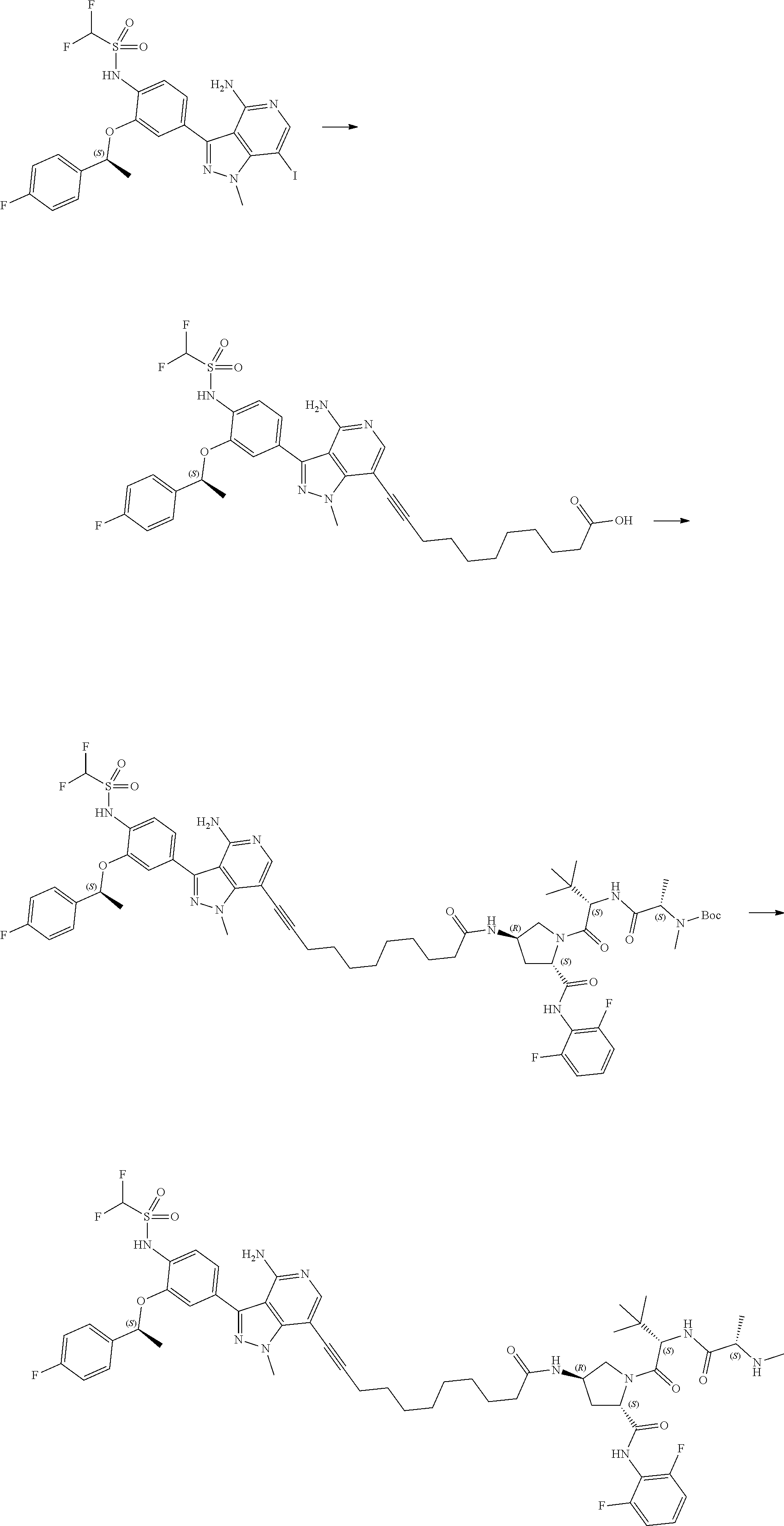

### Step 1: 11-{4-amino-3-[4-(difluoromethanesulfonamido)-3-[(1 S)-1-(4-fluorophenyl)ethoxy]phenyl]-1-methyl-1H-pyrazolo[4,3-c]pyridin-7-yl}undec-10-ynoic acid

A mixture of N-(4-{4-amino-7-iodo-1-methyl-1H-pyrazolo[4,3-c]pyridin-3-yl}-2-[(1S)-1-(4-fluoro phenyl)ethoxy]phenyl)-1,1-difluoromethanesulfonamide (500 mg, 809 μmol), undec-10-ynoic acid (177 mg, 971 μmol), Cul (154 mg, 808 μmol), Pd(PPh₃)₂Cl₂ (171 mg, 242 μmol) and Et₃N (820 mg, 8.10 mmol) in DMF (10 mL) was stirred at 130° C. under N₂ for 2 h. The mixture was concentrated under reduced pressure and the residue was diluted with water. The aqueous mixture was extracted with DCM (3×150 mL), and the combine organic phases were dried over Na₂SO₄ and concentrated. The crude residue was purified by Prep-HPLC to give the title product (104 mg, 19%) as a yellow solid. LCMS (method B): 2.16 min, m/z 672.3 [M+H]⁺.

### Step 2: tert-butyl N-[(1S)-1-{[(2S)-1-[(2S)-4-(11-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}undec-10-ynamido)-2-[(2,6-difluorophenyl)carbamoyl]pyrrolidin-1-yl]-3,3-dimethyl-1-oxobutan-2-yl]carbamoyl)ethyl]-N-methylcarbamate

A mixture of 11-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}undec-10-ynoic acid (29.87 mg, 44.3 μmol), tert-butyl N-[(1S)-1-{[(2S)-1-[(2S,4S)-4-amino-2-[(2,6-difluorophenyl)carbamoyl]pyrrolidin-1-yl]-3,3-dimethyl-1-oxobutan-2-yl]carbamoyl}ethyl]-N-methylcarbamate (20 mg, 37.0 μmol), HATU (16.92 mg, 44.4 μmol), and Et₃N (11.26 mg, 110 μmol) in MeCN (1.5 mL) was stirred at RT for 16 h. The mixture was concentrated under reduced pressure and the residue was diluted with water. The aqueous mixture was extracted with DCM (3×50 mL), and the combine organic phases were dried over Na₂SO₄ and concentrated. The crude residue was purified by Prep-TLC (DCM:MeOH=20:1) to give the title product (13.5 mg, 35%) as a yellow solid.

### Step 3: (2S)-4-(11-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}undec-10-ynamido)-N-(2,6-difluoro phenyl)-1-[(2S)-3,3-dimethyl-2-[(2S)-2-(methylamino)propanamido]butanoyl]pyrrolidine-2-carboxamide

A solution of tert-butyl N-[(1S)-1-{[(2S)-1-[(2S)-4-(11-{4-amino-3-[4-(difluoromethane sulfonamido)-3-[(1S)-1-(4-fluorophenyl)ethoxy]phenyl]-1-methyl-1H-pyrazolo[4,3-c]pyridin-7-yl}undec-10-ynamido)-2-[(2,6-difluorophenyl)carbamoyl]pyrrolidin-1-yl]-3,3-dimethyl-1-oxobutan-2-yl]carbamoyl}ethyl]-N-methylcarbamate (22.2 mg, 18.6 μmol) in HCO₂H (1 mL) was stirred at RT for 2 h. The mixture was concentrated under reduced pressure and the residue was diluted with water. The aqueous mixture was extracted with DCM (3×50 mL), and the combine organic phases were dried over Na₂SO₄ and concentrated. The crude residue was purified by Prep-TLC (DCM:MeOH=10:1) to give the title product (8.4 mg, 41%) as a grey solid. LCMS (method B): 1.89 min, m/z 547.4 [M+2H]²⁺. ¹H NMR (400 MHz, DMSO-d₆): 9.99 (s, 1H), 8.61 (d, J=7.9 Hz, 1H), 8.12 (d, J=7.5 Hz, 1H), 7.81 (s, 1H), 7.56 (s, 2H), 7.48 (s, 1H), 7.39 (d, J=8.1 Hz, 1H), 7.34 (s, 2H), 7.23 (s, 1H), 7.14 (d, J=9.4 Hz, 4H), 7.10 (t, J=52 Hz, 1H), 6.00 (s, 1H), 5.65 (d, J=6.5 Hz, 1H), 4.56 (d, J=6.0 Hz, 2H), 4.43 (d, J=7.9 Hz, 1H), 4.30 (s, 1H), 4.21 (s, 3H), 4.15 (s, 1H), 3.95 (d, J=7.0 Hz, 1H), 2.44 (s, 3H), 2.11 (s, 2H), 2.06 (t, J=7.6 Hz, 2H), 1.80 (d, J=11.0 Hz, 1H), 1.57 (d, J=6.4 Hz, 3H), 1.49 (s, 4H), 1.35 (d, J=6.7 Hz, 3H), 1.26 (dd, J=19.4, 6.1 Hz, 9H), 0.98 (s, 9H).

### Compound 1140: (2S)-4-(9-{4-amino-3-[4-(difluoromethanesulfonamido)-3-[(1 S)-1-(4-fluorophenyl)ethoxy]phenyl]-1-methyl-1H-pyrazolo[4,3-c]pyridin-7-yl}non-8-ynamido)-N-(2,6-difluorophenyl)-1-[(2S)-3,3-dimethyl-2-[(2S)-2-(methylamino)propanamido]butanoyl]pyrrolidine-2-carboxamide

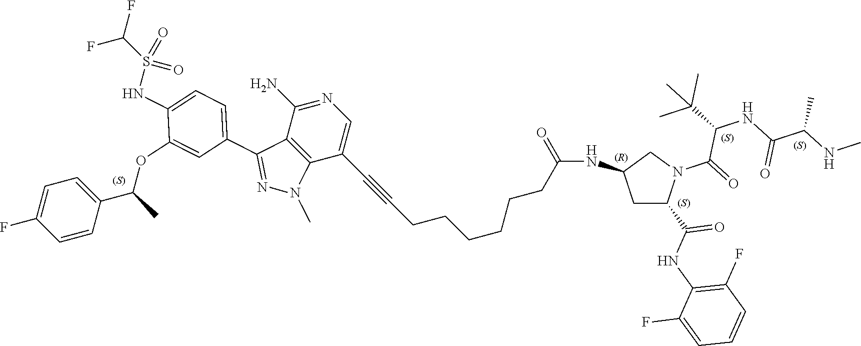

The title compound was prepared according to the procedure for the synthesis of Compound 1138 using non-8-ynoic acid in Step 1. The compound was obtained as a white solid (6.2 mg, 35%). LCMS (Method B): 1.82 min, m/z: 533.4 [M+2H]²⁺. ¹H NMR (400 MHz, DMSO-d₆): 9.98 (s, 1H), 8.61 (d, J=7.9 Hz, 1H), 8.13 (d, J=7.4 Hz, 1H), 7.82 (s, 1H), 7.56 (dd, J=8.5, 5.5 Hz, 2H), 7.46 (s, 2H), 7.39 (d, J=8.5 Hz, 2H), 7.34 (s, 2H), 7.22 (s, 1H), 7.16-7.13 (m, 3H), 7.11 (s, 1H), 6.95 (d, J=52.6 Hz, 1H), 6.05 (s, 1H), 5.65 (d, J=6.4 Hz, 1H), 4.57 (s, 1H), 4.43 (d, J=8.0 Hz, 1H), 4.34-4.27 (m, 1H), 4.22 (s, 3H), 4.15 (s, 1H), 3.95 (d, J=7.0 Hz, 1H), 3.25 (s, 2H), 2.48 (s, 2H), 2.44 (s, 2H), 2.11 (s, 3H), 2.08 (t, J=7.9 Hz, 2H), 1.81 (d, J=10.4 Hz, 1H), 1.57 (d, J=6.4 Hz, 4H), 1.42 (s, 2H), 1.35 (d, J=6.9 Hz, 3H), 1.23 (s, 4H), 0.97 (s, 9H).

### Intermediate J1: 2-{[2-(2,6-dioxopiperidin-3-yl)-1,3-dioxo-2,3-dihydro-1H-isoindol-4-yl]oxy}acetic acid

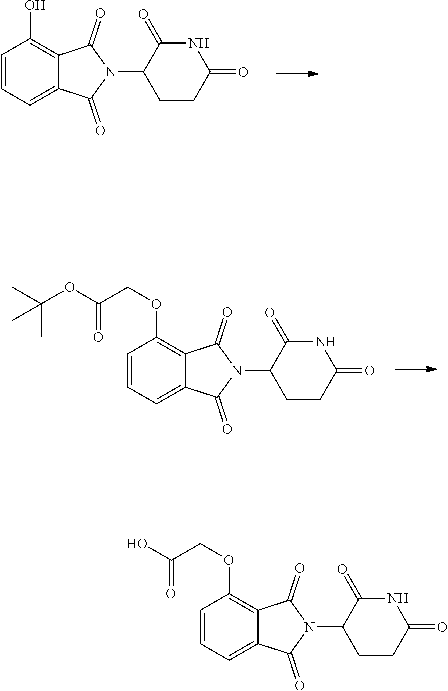 [SMILES: O=C1CCC(N2C(=O)c3cccc(O)c3C2=O)C(=O)N1 ; CC(C)(C)OC(=O)COc1cccc2c1C(=O)N(C1CCC(=O)NC1=O)C2=O ; O=C(O)COc1cccc2c1C(=O)N(C1CCC(=O)NC1=O)C2=O]

### Step 1: tert-butyl 2-{[2-(2,6-dioxopiperidin-3-yl)-1,3-dioxo-2,3-dihydro-1H-isoindol-4-yl]oxy}acetate

To a 0° C. solution of 2-(2,6-dioxopiperidin-3-yl)-4-hydroxy-2,3-dihydro-1H-isoindole-1,3-dione (2.0 g, 7.29 mmol) in THE (10 mL) were added tert-butyl 2-hydroxyacetate (963 mg, 7.29 mmol) and PPh₃ (2.48 g, 9.47 mmol). The mixture was stirred at 0° C. for 5 min before DIAD (1.91 g, 9.47 mmol) was added. The mixture was warmed to RT and stirred for 2 h. The mixture was filtered, and the filtrate was concentrated. The residue was purified by Reverse-Phase Chromatography to afford the title compound (1.30 g, 46%) as a white solid. LCMS (Method A): 3.09 min, m/z: 389.1 [M+H]⁺.

### Step 2: 2-{[2-(2,6-dioxopiperidin-3-yl)-1,3-dioxo-2,3-dihydro-1H-isoindol-4-yl]oxy}acetic acid

A solution of tert-butyl 2-{[2-(2,6-dioxopiperidin-3-yl)-1,3-dioxo-2,3-dihydro-1H-isoindol-4-yl]oxy}acetate (2.6 g, 6.69 mmol) in 4M HCl/Dioxane (10 mL) was stirred at RT for 1 h. The mixture was concentrated, and the residue was purified by reverse-phase chromatography to afford the title compound (400 mg, 18%) as a yellow solid. LCMS (Method A): 1.64 min, m/z: 333.1 [M+H]⁺.

### Intermediate J2: 2-{[2-(2,6-dioxopiperidin-3-yl)-1,3-dioxo-2,3-dihydro-1H-isoindol-5-yl]oxy}acetic acid

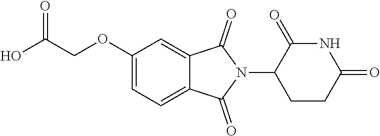 [SMILES: O=C(O)COc1ccc2c(c1)C(=O)N(C1CCC(=O)NC1=O)C2=O]

The title compound was prepared according to the procedure for Intermediate J1, utilising 2-(2,6-dioxopiperidin-3-yl)-5-hydroxy-2,3-dihydro-1H-isoindole-1,3-dione in Step 1. LCMS (Method A): 1.73 min, m/z: 333.1 [M+H]+.

### Compound 1097: N-(8-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}oct-7-yn-1-yl)-2-{[2-(2,6-dioxopiperidin-3-yl)-1,3-dioxo-2,3-dihydro-1H-isoindol-4-yl]oxy}acetamide

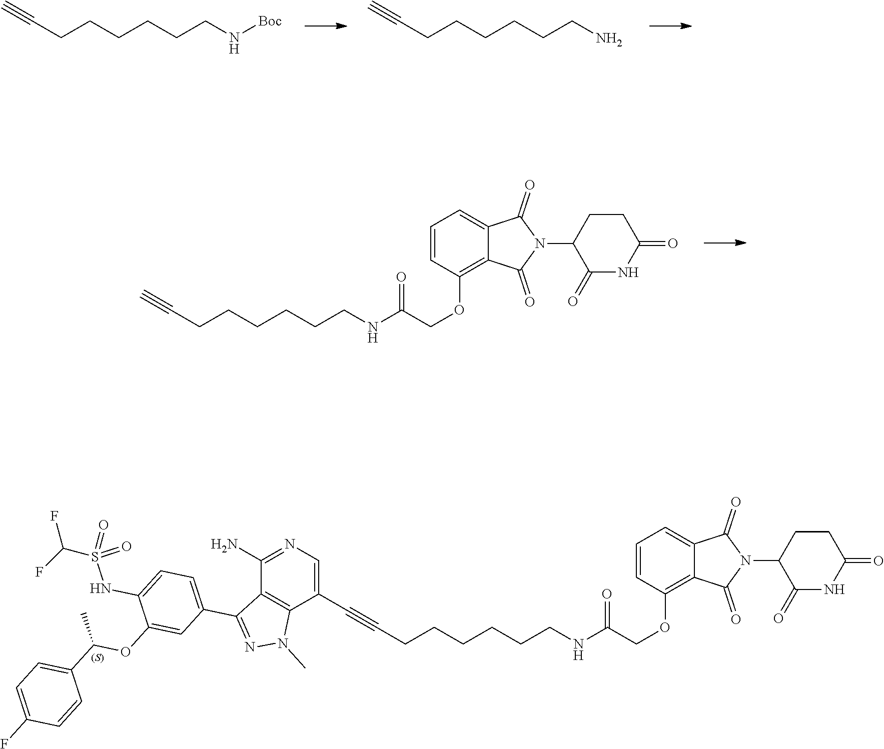

### Step 1: oct-7-yn-1-amine

A solution of tert-butyl N-(oct-7-yn-1-yl)carbamate (100 mg, 443 μmol) in 4M HCl/Dioxane (3 mL) was stirred at RT for 1 h. The mixture was concentrated, and the residue was poured into water and extracted with EtOAc. The combined organic phases were dried over Na₂SO₄ and concentrated to afford the title compound (55 mg, 99%) as a white solid.

### Step 2: 2-{[2-(2,6-dioxopiperidin-3-yl)-1,3-dioxo-2,3-dihydro-1H-isoindol-4-yl]oxy}-N-(oct-7-yn-1-yl)acetamide

A mixture of 2-{[2-(2,6-dioxopiperidin-3-yl)-1,3-dioxo-2,3-dihydro-1H-isoindol-4-yl]oxy}acetic acid (130 mg, 391 μmol), oct-7-yn-1-amine (48.9 mg, 391 μmol), HATU (178 mg, 469 μmol) and Et₃N (197 mg, 1.95 mmol) in MeCN (3 mL) was stirred at 0° C. under N₂ for 3 h. The reaction was concentrated, and the residue was poured into water and extracted with EtOAc. The combined organic phases were dried over Na₂SO₄ and concentrated. The residue was purified by Prep-TLC (DCM/MeOH=20/1) to afford the title compound (120 mg, 70%) as a yellow solid. LCMS (Method A): 4.25 min, m/z: 440.3 [M+H]⁺.

### Step 3: N-(8-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}oct-7-yn-1-yl)-2-{[2-(2,6-dioxopiperidin-3-yl)-1,3-dioxo-2,3-dihydro-1H-isoindol-4-yl]oxy}acetamide

A mixture of 2-{[2-(2,6-dioxopiperidin-3-yl)-1,3-dioxo-2,3-dihydro-1H-isoindol-4-yl]oxy}-N-(oct-7-yn-1-yl)acetamide (100 mg, 227 μmol), N-(4-{4-amino-7-iodo-1-methyl-1H-pyrazolo[4,3-c]pyridin-3-yl}-2-[(1S)-1-(4-fluorophenyl)ethoxy]phenyl)-1,1-difluoromethanesulfonamide (140 mg, 227 μmol), Cul (7.2 mg, 38.0 μmol) and Et₃N (77.0 mg, 762 μmol) in DMF (3 mL) was heated at 130° C. under N₂ overnight. The mixture was concentrated and the residue was purified by Prep-TLC (DCM/MeOH=20/1) to afford the title compound (56 mg, 33%) as a yellow solid. LCMS (Method A): 2.04 min, m/z: 929.2 [M+H]⁺. ¹H NMR (400 MHz, DMSO-d₆): 11.12 (s, 1H), 10.56 (s, 1H), 8.13 (s, 1H), 7.94 (t, J=5.7 Hz, 1H), 7.85-7.77 (m, 2H), 7.60-7.56 (m, 2H), 7.48 (d, J=7.3 Hz, 1H), 7.41-7.37 (m, 2H), 7.19-7.16 (m, 2H), 7.14 (d, J=2.7 Hz, 1H), 7.12 (d, J=2.1 Hz, 1H), 6.19 (s, 2H), 5.65 (q, J=6.3 Hz, 1H), 5.11 (dd, J=12.9, 5.4 Hz, 1H), 4.77 (s, 2H), 4.22 (s, 3H), 3.15 (dd, J=12.8, 6.2 Hz, 2H), 2.91-2.83 (m, 2H), 2.63-2.53 (m, 2H), 2.08-1.93 (m, 2H), 1.59 (d, J=3.6 Hz, 3H), 1.44 (qt, J=11.2, 5.9 Hz, 5H), 1.32 (qt, J=11.7, 7.0 Hz, 3H), 1.26-1.22 (m, 3H).

### Compound 1099: N-(4-{4-amino-7-[1-(1-{3-[4-(2-{[2-(2,6-dioxopiperidin-3-yl)-1,3-dioxo-2,3-dihydro-1H-isoindol-4-yl]oxy}acetyl)piperazin-1-yl]propyl}piperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

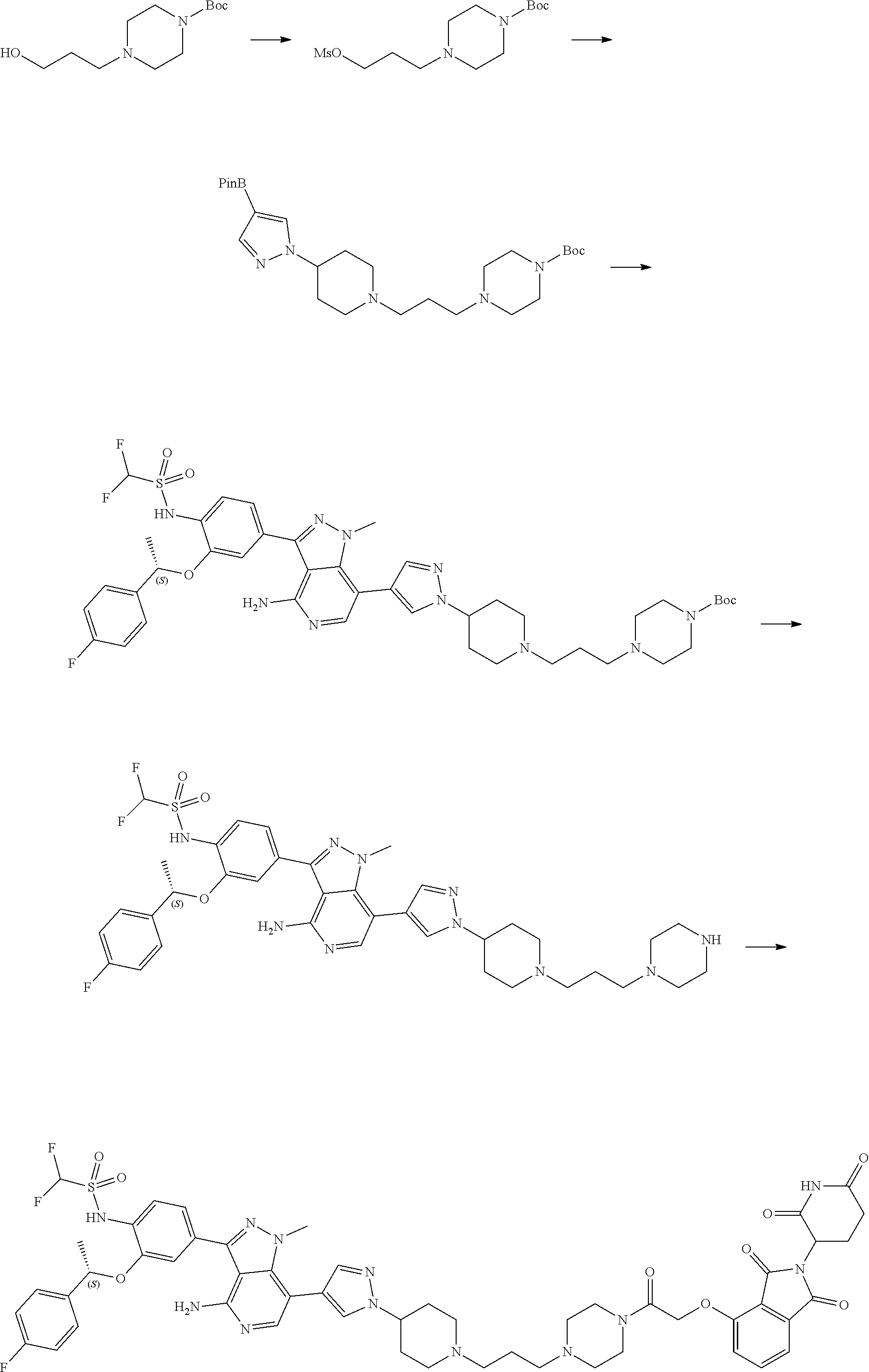

### Step 1: tert-butyl 4-[3-(methanesulfonyloxy)propyl]piperazine-1-carboxylate

To a mixture of tert-butyl 4-(3-hydroxypropyl)piperazine-1-carboxylate (3.0 g, 12.2 mmol) and Et₃N (3.69 g, 36.5 mmol) in DCM (30 mL) was added MsCl (2.07 g, 18.2 mmol), and the reaction was stirred at RT for 1 h. The mixture was partitioned between DCM (100 mL) and H₂O (100 mL), and the organic phase was separated, dried over Na₂SO₄ and concentrated to afford the title compound (4.00 g, 100%) as a yellow oil. LCMS (Method A): 1.35 min, m/z: 323.1 [M+H]⁺.

### Step 2: tert-butyl 4-(3-{4-[4-(4,4,5,5-tetramethyl-1,3,2-dioxaborolan-2-yl)-1H-pyrazol-1-yl]piperidin-1-yl}propyl)piperazine-1-carboxylate

A mixture of 4-[4-(4,4,5,5-tetramethyl-1,3,2-dioxaborolan-2-yl)-1H-pyrazol-1-yl]piperidine (3.0 g, 10.8 mmol), tert-butyl 4-[3-(methanesulfonyloxy)propyl]piperazine-1-carboxylate (3.48 g, 10.8 mmol) and Cs₂CO₃ (10.5 g, 32.4 mmol) in MeCN (50 mL) was stirred at 80° C. overnight. Once cooled, the mixture was diluted with EtOAc (100 mL) and washed with water (100 mL). The organic phase was dried over Na2SO4 and concentrated to afford the title compound (5.0 g, 92%) as a yellow solid. LCMS (Method A): 1.97 min, m/z: 504.4 [M+H]⁺.

### Step 3: tert-butyl 4-{3-[4-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)piperidin-1-yl]propyl}piperazine-1-carboxylate

A mixture of N-(4-{4-amino-7-iodo-1-methyl-1H-pyrazolo[4,3-c]pyridin-3-yl}-2-[(1S)-1-(4-fluoro phenyl)ethoxy]phenyl)-1,1-difluoromethanesulfonamide (1.2 g, 1.94 mmol), tert-butyl 4-(3-{4-[4-(4,4,5,5-tetramethyl-1,3,2-dioxaborolan-2-yl)-1H-pyrazol-1-yl]piperidin-1-yl}propyl)piperazine-1-carboxylate (1.26 g, 2.52 mmol), K₂CO₃ (801 mg, 5.81 mmol) and Pd(dppfCl₂ (160 mg, 0.19 mmol) in 80% aq. 1,4-dioxane (5 mL) was stirred at 100° C. for 16 h. The mixture was concentrated, and the residue was purified by column chromatography (DCM:MeOH, 20:1) to afford the title compound (700 mg, 42%) as a brown solid. LCMS (Method A): 2.17 min, m/z: 867.3 [M+H]⁺.

### Step 4: N-{4-[4-amino-1-methyl-7-(1-{1-[3-(piperazin-1-yl)propyl]piperidin-4-yl}-1H-pyrazol-4-yl)-1H-pyrazolo[4,3-c]pyridin-3-yl]-2-[(1 S)-1-(4-fluorophenyl)ethoxy]phenyl}-1,1-difluoromethanesulfonamide

To a solution of tert-butyl 4-{3-[4-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)piperidin-1-yl]propyl}piperazine-1-carboxylate (900 mg, 1.03 mmol) in DCM (40 mL) was added 4M HCl in dioxane (2 mL), and the reaction was stirred at RT for 5 min. The mixture was concentrated under reduced pressure, and the residue was neutralized to pH 7-8 with sat. aq. Na₂CO₃. The mixture was purified by RP-column to afford the title compound (180 mg, 23%) as a gray solid. LCMS (Method A): 1.07 min. m/z 768.0 [M+H]⁺.

### Step 5: N-(4-{4-amino-7-[1-(1-{3-[4-(2-{[2-(2,6-dioxopiperidin-3-yl)-1,3-dioxo-2,3-dihydro-1H-isoindol-4-yl]oxy}acetyl)piperazin-1-yl]propyl}piperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

A mixture of 2-{[2-(2,6-dioxopiperidin-3-yl)-1,3-dioxo-2,3-dihydro-1H-isoindol-4-yl]oxy}acetic acid (57.1 mg, 0.1720 mmol), Et₃N (57.9 mg, 0.5736 mmol) and HATU (76.2 mg, 0.2007 mmol) in MeCN (4 mL) was stirred at RT for 5 min. Then, N-{4-[4-amino-1-methyl-7-(1-{1-[3-(piperazin-1-yl)propyl]piperidin-4-yl}-1H-pyrazol-4-yl)-1H-pyrazolo[4,3-c]pyridin-3-yl]-2-[(1 S)-1-(4-fluorophenyl)ethoxy]phenyl}-1,1-difluoromethanesulfonamide (110 mg, 0.1434 mmol) was added, and the reaction was stirred at RT for 3 h. Water was added, then the aqueous mixture was extracted with EtOAc (3×30 mL). The combined organic layers were washed with brine, dried (Na₂SO₄) and concentrated. The residue was purified by prep-TLC and prep-HPLC to afford the title compound (6 mg, 2%) as a white solid. LCMS (Method A): 2.46 min, m/z: 541.4 [M+2H]²⁺¹H NMR (400 MHz, DMSO-d₆): 11.12 (s, 1H), 8.17 (s, 1H), 8.01 (s, 1H), 7.79 (t, J=8.0 Hz, 1H), 7.61 (s, 1H), 7.55 (dd, J=8.5, 5.0 Hz, 3H), 7.47 (d, J=7.2 Hz, 1H), 7.37 (dd, J=15.1, 8.3 Hz, 2H), 7.16 (t, J=8.7 Hz, 2H), 7.02 (d, J=8.2 Hz, 1H), 6.97 (s, 1H), 6.58 (d, J=44.6 Hz, 1H), 5.67 (d, J=7.3 Hz, 3H), 5.20 (s, 2H), 5.12 (dd, J=12.9, 5.4 Hz, 1H), 4.26 (s, 1H), 3.70 (s, 4H), 3.12-3.05 (m, 3H), 2.92 (t, J=15.4 Hz, 1H), 2.66 (d, J=22.3 Hz, 2H), 2.38 (s, 4H), 2.26 (s, 2H), 2.05 (dd, J=22.9, 12.1 Hz, 5H), 1.68 (s, 2H), 1.56 (d, J=6.3 Hz, 3H).

### Compound 1091: N-(4-{4-amino-7-[1-(1-{3-[4-(2-{[2-(2,6-dioxopiperidin-3-yl)-1,3-dioxo-2,3-dihydro-1H-isoindol-5-yl]oxy}acetyl)piperazin-1-yl]propyl}piperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

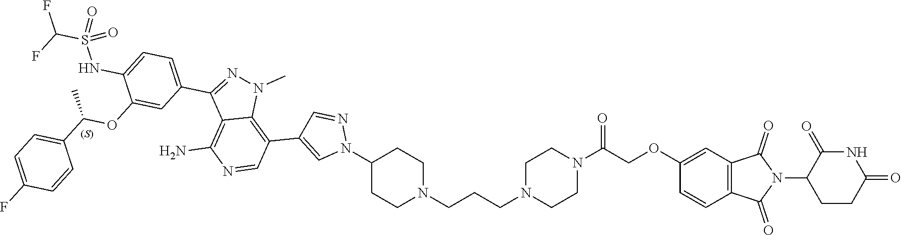

The title compound was prepared according to the procedure for Compound 1099, utilising Intermediate J2 in Step 5. LCMS (Method A): 2.42 min, m/z: 1082.3 [M+H]⁺. ¹H NMR (400 MHz, DMSO-d₆): 11.12 (s, 1H), 8.03 (s, 1H), 7.86 (d, J=8.3 Hz, 1H), 7.65 (s, 1H), 7.57 (q, J=5.1, 4.6 Hz, 3H), 7.45 (d, J=2.3 Hz, 1H), 7.42-7.34 (m, 2H), 7.18 (t, J=8.8 Hz, 2H), 7.12-7.04 (m, 2H), 6.86 (d, J=107.6 Hz, 1H), 5.71 (d, J=7.4 Hz, 2H), 5.67 (s, 1H), 5.14 (d, J=11.4 Hz, 3H), 4.42 (s, 1H), 3.71 (s, 3H), 3.51 (s, 5H), 3.00-2.86 (m, 2H), 2.81 (s, 3H), 2.70-2.58 (m, 3H), 2.44 (s, 5H), 2.25 (s, 4H), 2.13-2.02 (m, 1H), 1.86 (d, J=8.4 Hz, 2H), 1.58 (d, J=6.2 Hz, 3H).

### Compound 1082: N-[4-(4-amino-7-{1-[1-(2-{[2-(2,6-dioxopiperidin-3-yl)-1,3-dioxo-2,3-dihydro-1H-isoindol-4-yl]oxy}acetyl)piperidin-4-yl]-1H-pyrazol-4-yl}-1-methyl-1H-pyrazolo[4,3-c]pyridin-3-yl)-²-[(1S)-1-(4-fluorophenyl)ethoxy]phenyl]-1,1-difluoromethanesulfonamide

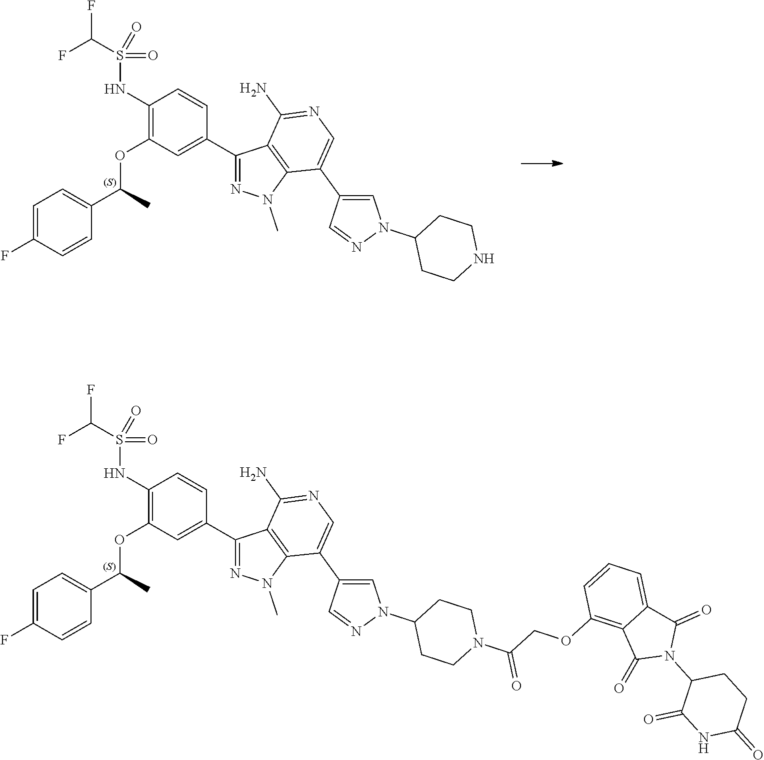

A mixture of N-(4-{4-amino-1-methyl-7-[1-(piperidin-4-yl)-1H-pyrazol-4-yl]-1H-pyrazolo[4,3-c]pyridin-3-yl}-2-[(1S)-1-(4-fluorophenyl)ethoxy]phenyl)-1,1-difluoromethanesulfonamide (50 mg, 78.0 μmol), 2-{[2-(2,6-dioxopiperidin-3-yl)-1,3-dioxo-2,3-dihydro-1H-isoindol-4-yl]oxy}acetic acid (93 mg, 279 μmol), HATU (75 mg, 197 μmol) and Et₃N (0.5 mL, 4.94 μmol) in MeCN (2 mL) was stirred at RT for 2 h. The mixture was concentrated, and the residue was purified by Reverse-Phase chromatography to afford the title compound (12.0 mg, 16%) as a yellow solid. LCMS (Method A): 3.68 min, m/z: 956.1 [M+H]⁺. ¹H NMR (400 MHz, DMSO-d₆): 11.11 (s, 1H), 10.59 (s, 1H), 8.14 (s, 1H), 7.74 (s, 2H), 7.66 (s, 1H), 7.62-7.56 (m, 2H), 7.49-7.43 (m, 2H), 7.38 (d, J=8.6 Hz, 1H), 7.29 (s, 1H), 7.24-7.13 (m, 5H), 7.04 (d, J=6.3 Hz, 1H), 6.66 (s, 1H), 5.66 (q, J=6.1 Hz, 1H), 5.27 (s, 2H), 5.11 (dd, J=12.9, 5.3 Hz, 1H), 4.58 (s, 1H), 4.42 (d, J=12.8 Hz, 1H), 3.95 (d, J=13.7 Hz, 1H), 3.76 (s, 3H), 2.89 (t, J=13.0 Hz, 3H), 2.18 (d, J=30.1 Hz, 4H), 2.01 (d, J=7.7 Hz, 3H), 1.87 (d, J=12.0 Hz, 2H), 1.60 (d, J=6.2 Hz, 3H), 1.47 (d, J=7.8 Hz, 2H).

### Compound 1085: N-[4-(4-amino-7-{1-[1-(2-{[2-(2,6-dioxopiperidin-3-yl)-1,3-dioxo-2,3-dihydro-1H-isoindol-5-yl]oxy}acetyl)piperidin-4-yl]-1H-pyrazol-4-yl}-1-methyl-1H-pyrazolo[4,3-c]pyridin-3-yl)-2-[(1 S)-1-(4-fluorophenyl)ethoxy]phenyl]-1,1-difluoromethanesulfonamide

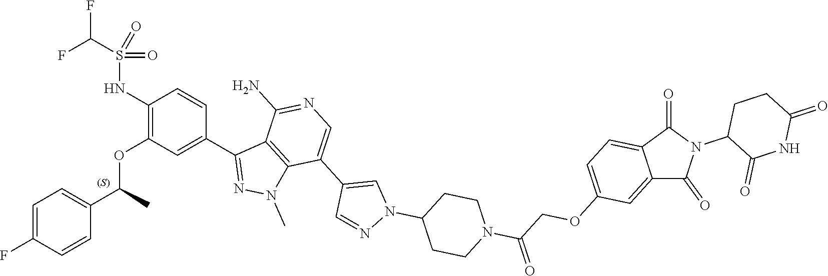

The title compound was prepared according to the procedure for Compound 1082, utilising Intermediate J2 in Step 1. LCMS: (Method A) 2.71 min, m/z: 955.2 [M+H]⁺. ¹ HNMR (400 MHz, DMSO-d₆): 11.11 (s, 1H), 8.03 (s, 1H), 7.84 (d, J=8.4 Hz, 1H), 7.63 (s, 1H), 7.58-7.54 (m, 3H), 7.46 (d, J=2.4 Hz, 1H), 7.41-7.35 (m, 2H), 7.18-7.09 (m, 4H), 6.97 (t, J=52.8 Hz, 1H), 5.90 (s, 2H), 5.65 (q, J=6.4 Hz, 1H), 5.19 (d, J=4.8 Hz, 2H), 5.11 (dd, J=5.2 Hz, 12.8 Hz, 1H), 4.56-4.40 (m, 2H), 3.95 (d, J=13.6 Hz, 1H), 3.70 (s, 3H), 3.27-3.22 (m, 1H), 2.90-2.84 (m, 2H), 2.67-2.55 (m, 2H), 2.14-2.03 (m, 4H), 1.91-1.84 (m, 1H), 1.57 (d, J=6.4 Hz, 3H).

### Intermediate K1: 4-[(2,6-dioxopiperidin-3-yl)carbamoyl]benzoic acid

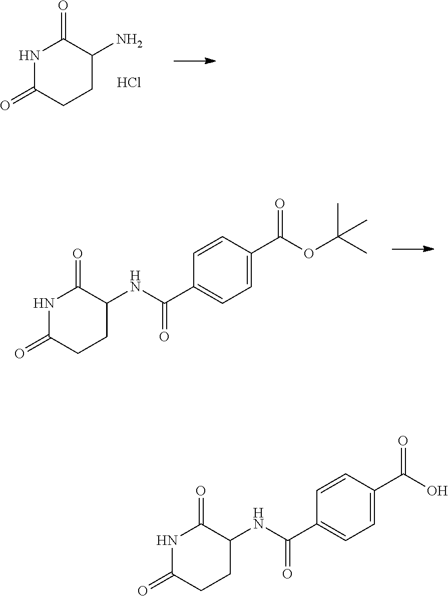 [SMILES: NC1CCC(=O)NC1=O ; Cl ; CC(C)(C)OC(=O)c1ccc(C(=O)NC2CCC(=O)NC2=O)cc1 ; O=C1CCC(NC(=O)c2ccc(C(=O)O)cc2)C(=O)N1]

### Step 1: tert-butyl 4-[(2,6-dioxopiperidin-3-yl)carbamoyl]benzoate

To a solution of 4-[(tert-butoxy)carbonyl]benzoic acid (900 mg, 4.04 mmol) in MeCN (10 mL) were added 3-aminopiperidine-2,6-dione (450 mg, 3.51 mmol), Et₃N (1.22 g, 12.1 mmol) and HATU (1.69 g, 4.44 mmol), and the resulting mixture was stirred at RT for 2 h. The mixture was concentrated, and the residue was purified by column chromatography (DCM: MeOH, 50:1) to afford the title compound (1.10 g, 82%). LCMS (Method A2): 2.99 min, m/z: 333.3 [M+H]⁺.

### Step 2: 4-[(2,6-dioxopiperidin-3-yl)carbamoyl]benzoic acid

A solution of tert-butyl 4-[(2,6-dioxopiperidin-3-yl)carbamoyl]benzoate (500 mg, 1.50 mmol) in HCO₂H (8 mL) was stirred at RT for 2 h. The mixture was concentrated to afford the title compound (400 mg, 97%). LCMS (Method A2): 3.41 min, m/z: 277.5 [M+H]⁺.

Intermediate K2: 3-[(2,6-dioxopiperidin-3-yl)carbamoyl]benzoic acid

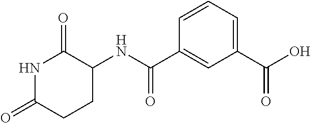 [SMILES: O=C1CCC(NC(=O)c2cccc(C(=O)O)c2)C(=O)N1]

The title compound was prepared from 3-[(tert-butoxy)carbonyl]benzoic acid according to the procedure for the synthesis of Intermediate K1. LCMS (method A): 1.15 min, m/z 277.1 [M+H]⁺

### Intermediate K3: 2-(2,6-dioxopiperidin-3-yl)-1-oxo-2,3-dihydro-1H-isoindole-5-carboxylic acid

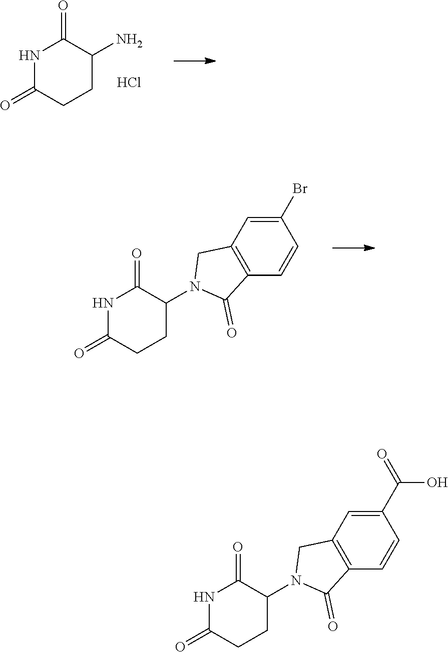 [SMILES: NC1CCC(=O)NC1=O ; Cl ; O=C1CCC(N2Cc3cc(Br)ccc3C2=O)C(=O)N1 ; O=C1CCC(N2Cc3cc(C(=O)O)ccc3C2=O)C(=O)N1]

### Step 1: 3-(5-bromo-1-oxo-2,3-dihydro-1H-isoindol-2-yl)piperidine-2,6-dione

A mixture of methyl 4-bromo-2-(bromomethyl)benzoate (3.0 g, 9.74 mmol), 3-aminopiperidine-2,6-dione (1.60 g, 12.4 mmol) and Et₃N (4.0 g, 39.5 mmol) in DMF (40 mL) was stirred at 120° C. overnight. Water was added, and the mixture was extracted with DCM:MeOH (10:1). The organics were concentrated, and the residue was purified by column chromatography to afford the title compound (1.23 g, 39%) as a yellow solid. LCMS (Method A2): 2.58 min, m/z: 323.0 [M+H]⁺.

### Step 2: 2-(2,6-dioxopiperidin-3-yl)-1-oxo-2,3-dihydro-1H-isoindole-5-carboxylic acid

A mixture of 3-(5-bromo-1-oxo-2,3-dihydro-1H-isoindol-2-yl)piperidine-2,6-dione (500 mg, 1.54 mmol), HCO₂H (160 mg, 3.47 mmol), Pd(OAc)₂ (34.5 mg, 154 μmol), Et₃N (623 mg, 6.16 mmol), Xantphos (178 mg, 308 μmol) and DCC (317 mg, 1.54 mmol) in DMF (15 mL) was stirred at 100° C. under N₂ overnight. The mixture was concentrated and filtered to afford the title compound (409 mg, 92%) as a green solid. LCMS (Method A2): 1.14 min, m/z: 289.0 [M+H]⁺.

### Intermediate K4: 4-methoxy-3-(2-oxo-1,3-diazinan-1-yl)benzoic acid

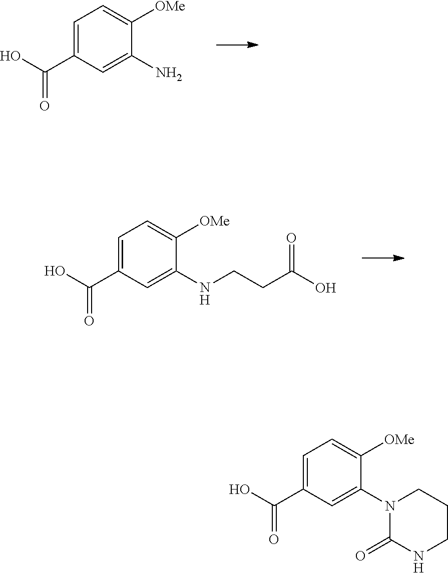 [SMILES: COc1ccc(C(=O)O)cc1N ; COc1ccc(C(=O)O)cc1NCCC(=O)O ; COc1ccc(C(=O)O)cc1N1CCCNC1=O]

### Step 1: 3-[(2-carboxyethyl)amino]-4-methoxybenzoic acid

A mixture of 3-amino-4-methoxybenzoic acid (200 mg, 1.19 mmol) and Acrylic acid (2 mL) was stirred at 100° C. overnight. The mixture was concentrated, and the residue was purified by column chromatography to afford the title compound (4.19 g, 98%) as a yellow solid. LCMS (Method A2): 1.92 min, m/z: 240.1 [M+H]⁺.

### Step 2: 4-methoxy-3-(2-oxo-1,3-diazinan-1-yl)benzoic acid

A mixture of 3-[(2-carboxyethyl)amino]-4-methoxybenzoic acid (1.0 g, 4.18 mmol) and urea (0.50 g, 8.37 mmol) in AcOH (15 mL) was stirred at 120° C. overnight. The mixture was concentrated, and the residue was purified by column chromatography to afford the title compound (30 mg, 3%) as a yellow solid. LCMS (Method A2): 1.38 min, m/z: 265.1 [M+H]⁺.

Intermediate K5: 3-(2,4-dioxo-1,3-diazinan-1-yl)-1-benzofuran-6-carboxylic acid

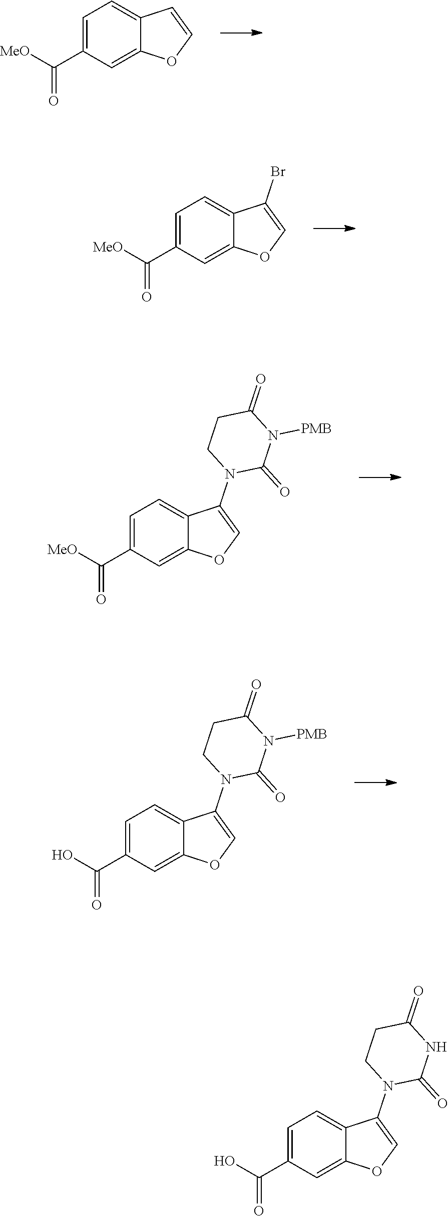

### Step 1: methyl 3-bromo-1-benzofuran-6-carboxylate

A mixture of methyl 1-benzofuran-6-carboxylate (200 mg, 1.13 mmol) and Br₂ (540 mg, 3.38 mmol) in DCM (5 mL) was stirred at RT under N₂ for 12 h. The reaction was quenched with aq. Na₂S₂O₃ then extracted with DCM. The organic phases were combined, dried over Na₂SO₄, and concentrated. The residue was dissolved in THE (10 mL) and an aq. solution of Cs₂CO₃ (736 mg, 2.26 mmol) was added. The resultant mixture was stirred at room temperature for 30 min, then diluted with H₂O and extracted with EtOAc (3×10 mL). The combined organic phases were dried over Na₂SO₄ and concentrated. The crude residue was purified by prep-TLC (PE:EA=50:1) to give the title compound (200 mg, 69%) as white solid.

### Step 2: methyl 3-{3-[(4-methoxyphenyl)methyl]-2,4-dioxo-1,3-diazinan-1-yl}-1-benzofuran-6-carboxylate

A mixture of methyl 3-bromo-1-benzofuran-6-carboxylate (200 mg, 784 μmol) 3-[(4-methoxyphenyl)methyl]-1,3-diazinane-2,4-dione (183 mg, 784 μmol), K₃PO₄ (498 mg, 2.35 mmol), Cul (149 mg, 784 μmol) and trans-1,2-diaminocyclohexane (89.5 mg, 784 μmol) in dioxane (10 mL) was stirred at 120° C. overnight. The mixture was diluted with H₂O (30 mL) and extracted with EtOAc (3×20 mL). The combined organic layers were dried (Na₂SO₄) and concentrated. The residue was purified by prep-TLC (DCM:MeOH, 20:1) to afford the title product (200 mg, 62%) as a yellow solid. LCMS (method C): 4.32 min, m/z 409.5 [M+H]⁺.

### Step 3: 3-{3-[(4-methoxyphenyl)methyl]-2,4-dioxo-1,3-diazinan-1-yl}-1-benzofuran-6-carboxylic acid

A solution of methyl 3-{3-[(4-methoxyphenyl)methyl]-2,4-dioxo-1,3-diazinan-1-yl}-1-benzofuran-6-carboxylate (300 mg, 734 μmol) in 4M HCl in 1,4-dioxane (5 mL) was stirred at RT for 2 h. The mixture was diluted with H₂O (30 mL) and extracted with EtOAc (3×20 mL). The combined organic layers were dried (Na₂SO₄) and concentrated. The residue was purified by prep-TLC (DCM:MeOH, 20:1) to afford the title product (130 mg, 45%) as a yellow solid. LCMS (method C): 3.80 min, m/z 395.6 [M+H]⁺.

### Step 4: 3-(2,4-dioxo-1,3-diazinan-1-yl)-1-benzofuran-6-carboxylic acid

A mixture of 3-{3-[(4-methoxyphenyl)methyl]-2,4-dioxo-1,3-diazinan-1-yl}-1-benzofuran-6-carboxylic acid (120 mg, 304 μmol), in TFA (3 mL) and CF₃SO₃H (2 mL) was stirred at 0° C. for 2 h. The mixture was diluted with H₂O (30 mL) and extracted with EtOAc (3×20 mL). The combined organic layers were dried (Na₂SO₄) and concentrated. The residue was purified by column chromatography to afford the title product (30.0 mg, 36%) as a brown solid. LCMS (method C): 3.25 min, m/z 275.5 [M+H]⁺.

Intermediate K5′: 3-[(4-methoxyphenyl)methyl]-1,3-diazinane-2,4-dione

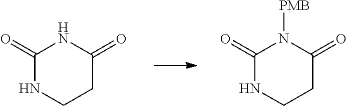

### Step 1: 3-[(4-methoxyphenyl)methyl]-1,3-diazinane-2,4-dione

A mixture of 1,3-diazinane-2,4-dione (150 mg, 1.31 mmol) Cs₂CO₃ (1.28 g, 3.93 mmol) and 4-methoxybenzyl chloride (245 mg, 1.57 mmol) in DMF (5 mL) was stirred at 80° C. overnight. The mixture was diluted with H₂O (30 mL) and extracted with EtOAc (3×20 mL). The combined organic layers were dried (Na₂SO₄) and concentrated under reduced pressure. The crude residue was purified by prep-TLC (DCM:MeOH, 20:1) to afford the title product (120 mg, 39%) as white solid. LCMS (method A2): 2.48 min, m/z 235.1 [M+H]⁺.

### Compound 1086: 4-[4-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)piperidine-1-carbonyl]-N-(2,6-dioxopiperidin-3-yl)benzamide

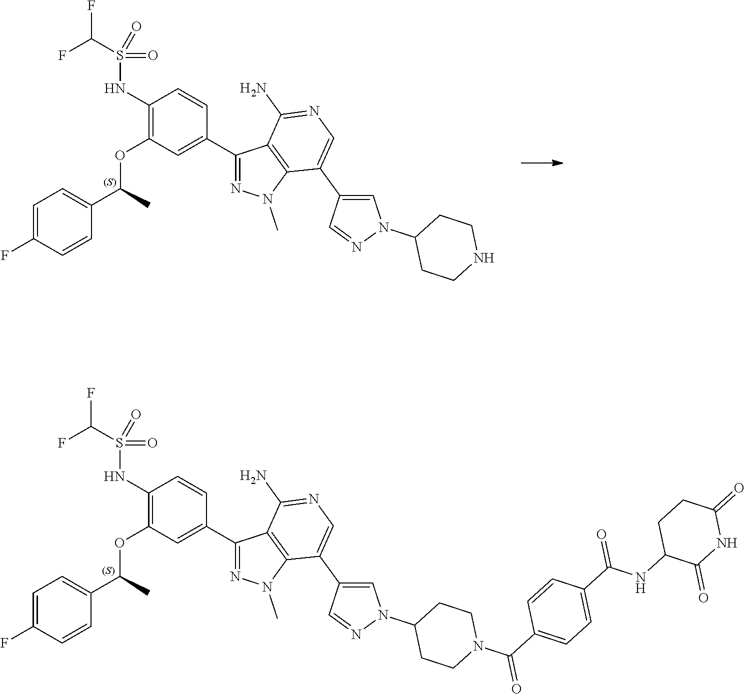

### Step 1: 4-[4-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)piperidine-1-carbonyl]-N-(2,6-dioxopiperidin-3-yl)benzamide

A mixture of 4-[(2,6-dioxopiperidin-3-yl)carbamoyl]benzoic acid (25 mg, 81.4 μmol), N-(4-{4-amino-1-methyl-7-[1-(piperidin-4-yl)-1H-pyrazol-4-yl]-1H-pyrazolo[4,3-c]pyridin-3-yl}-2-[(1 S)-1-(4-fluorophenyl)ethoxy]phenyl)-1,1-difluoromethanesulfonamide (52.1 mg, 81.4 μmol), HATU (30.9 g, 81.4 μmol) and Et₃N (24.6 mg, 244 μmol) in MeCN (10 mL) was stirred at RT for 2 h. The mixture was concentrate, and the residue was purified by prep-TLC (PE:EtOAc, 1:1) to afford the title compound (15 mg, 21%). LCMS (Method A2): 2.58 min, m/z: 899.2 [M+H]⁺. ¹H NMR (400 MHz, MeOD-d₄): 7.98 (d, J=8.4 Hz, 2H), 7.94 (d, J=0.7 Hz, 1H), 7.65 (d, J=0.7 Hz, 1H), 7.59 (d, J=8.3 Hz, 2H), 7.52 (s, 4H), 7.17 (dd, J=8.1, 1.8 Hz, 1H), 7.10 (d, J=1.8 Hz, 1H), 7.05 (t, J=8.8 Hz, 2H), 6.68 (t, J=53.3 Hz, 1H), 5.57 (d, J=6.4 Hz, 1H), 4.66-4.52 (m, 1H), 3.73 (s, 3H), 2.85-2.78 (m, 1H), 2.74 (dd, J=4.5, 3.0 Hz, 1H), 2.26-2.18 (m, 3H), 2.12 (s, 2H), 1.69 (d, J=6.4 Hz, 3H), 1.40 (s, 3H), 1.28 (d, J=1.9 Hz, 8H).

The following compounds were similarly prepared from Compound 59 and the indicated Intermediate K according to the procedure for the synthesis of Compound 1086.

TABLE 17
Compounds 1084, 1096, 1092 and 1117

| Example | | |
|---|---|---|
| No. | Structure | Compound Name and Analytical Data |
| 1084 | 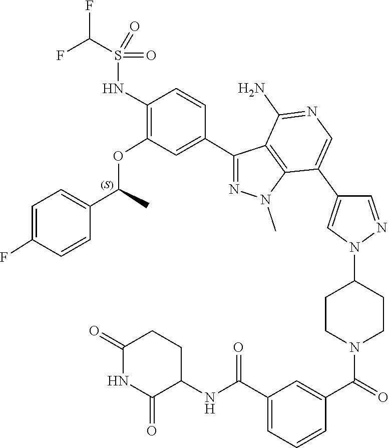 | 3-[4-(4-{4-amino-3-[4-(difluoromethanesulfonamido)-3-[(1S)- 1-(4-fluorophenyl)ethoxy]phenyl]-1-methyl-1H-pyrazolo [4,3-c]pyridin-7-yl}-1H-pyrazol-1-yl)piperidine-1-carbonyl]- N-(2,6-dioxopiperidin-3-yl)benzamide From Intermediate K2. LCMS (Method A2): 2.68 min, m/z: 899.2 [M + H]⁺. ¹H NMR (400 MHz, DMSO-d₆): 10.88 (s, 1H), 9.04 (d, J = 8.3 Hz, 1H), 8.09 (s, 1H), 8.02-7.94 (m, 2H), 7.67-7.52 (m, 6H), 7.40 (d, J = 8.0 Hz, 1H), 7.21-7.06 (m, 4H), 6.91 (d, J = 52.8 Hz, 1H), 5.94 (s, 1H), 5.66 (d, J = 6.4 Hz, 1H), 4.80 (ddd, J = 13.0, 8.2, 5.3 Hz, 1H), 4.69-4.48 (m, 2H), 3.11 (d, J = 41.2 Hz, 2H), 2.81 (ddd, J = 17.9, 13.2, 5.4 Hz, 1H), 2.57 (d, J = 3.9 Hz, 1H), 2.18 (dd, J = 12.8, 4.7 Hz, 3H), 2.08-1.91 (m, 4H), 1.58 (d, J = 6.2 Hz, 3H). |
| 1096 | 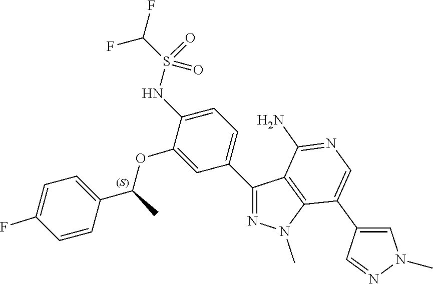 | N-{4-[4-amino-7-(1-{1-[2-(2,6-dioxopiperidin-3-yl)-1-oxo- 2,3-dihydro-1H-isoindole-5-carbonyl]piperidin-4-yl}-1H- pyrazol-4-yl)-1-methyl-1H-pyrazolo[4,3-c]pyridin-3-yl]-2- [(1S)-1-(4-fluorophenyl)ethoxy]phenyl}-1,1- difluoromethanesulfonamide From Intermediate K3. LCMS (Method A2): 2.74 min, m/z: 911.2 [M + H]⁺. ¹H NMR (400 MHz, DMSO-d₆): 10.90 (s, 1H), 8.04 (s, 1H), 7.92 (s, 1H), 7.69 (d, J = 7.8 Hz, 1H), 7.58 (s, 1H), 7.51 (s, 1H), 7.48-7.38 (m, 4H), 7.26 (d, J = 8.1 Hz, 1H), 7.08-7.00 (m, 2H), 6.96-6.87 (m, 2H), 5.54 (dd, J = 12.4, 5.9 Hz, 3H), 5.03 (dd, J = 13.3, 5.1 Hz, 1H), 4.45-4.36 (m, 2H), 4.28 (d, J = 17.6 Hz, 1H), 3.57 (s, 3H), 2.83-2.75 (m, 1H), 2.54-2.45 (m, 1H), 2.43 (s, 2H), 2.30 (dd, J = 13.1, 4.6 Hz, 1H), 2.26-2.01 (m, 2H), 1.99-1.79 (m, 4H), 1.44 (d, J = 6.3 Hz, 3H). |
| 1092 | 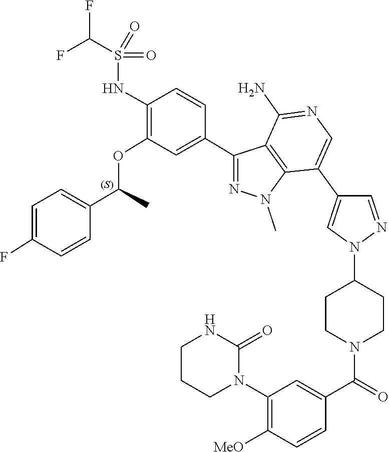 | N-{4-[4-amino-7-(1-{1-[3-(2,4-dioxo-1,3-diazinan-1-yl)- 4-methoxybenzoyl]piperidin-4-yl}-1H-pyrazol-4-yl)-1- methyl-1H-pyrazolo[4,3-c]pyridin-3-yl]-2-[(1S)-1-(4- fluorophenyl)ethoxy]phenyl}-1,1- difluoromethanesulfonamide From Intermediate K4. LCMS (Method A2): 2.72 min, m/z: 887.2 [M + H]⁺. ¹H NMR (400 MHz, DMSO-d₆): 10.35 (s, 1H), 8.03 (s, 1H), 7.62 (s, 1H), 7.58-7.54 (m, 3H), 7.46-7.39 (m, 3H), 7.19-7.09 (m, 6H), 6.93 (d, J = 52.7 Hz, 1H), 5.78-5.72 (m, 2H), 5.65 (q, J = 6.3 Hz, 1H), 4.53 (ddt, J = 11.2, 8.1, 4.1 Hz, 2H), 3.85 (s, 3H), 3.69 (s, 3H), 3.61 (t, J = 6.7 Hz, 3H), 3.17 (s, 1H), 2.69 (t, J = 6.7 Hz, 2H), 1.58 (d, J = 6.2 Hz, 3H), 1.24 (d, J = 4.9 Hz, 4H). |
| 1117 | 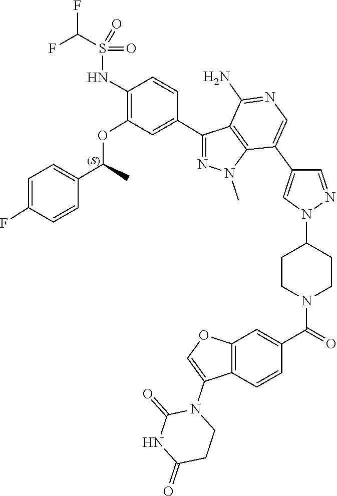 | N-{4-[4-amino-7-(1-{1-[3-(2,4-dioxo-1,3-diazinan-1-yl)-1- benzofuran-6-carbonyl]piperidin-4-yl}-1H-pyrazol-4-yl)-1- methyl-1H-pyrazolo[4,3-c]pyridin-3-yl]-2-[(1S)-1- (4-fluorophenyl)ethoxy]phenyl}-1,1- difluoromethanesulfonamide From Intermediate K5. LCMS (Method A2): 2.88 min, m/z: 897.3 [M + H]⁺. ¹H NMR (400 MHz, DMSO-d₆): 8.24 (s, 1H), 8.03 (s, 1H), 7.69 (d, J = 16.4 Hz, 3H), 7.61 (s, 1H), 7.56-7.24 (m, 6H), 7.12 (t, J = 8.9 Hz, 2H), 6.95-6.79 (m, 2H), 4.54 (s, 1H), 3.87 (d, J = 6.6 Hz, 3H), 3.67 (s, 3H), 2.79 (d, J = 7.6 Hz, 2H), 2.67 (s, 2H), 2.33 (s, 2H), 2.00 (s, 4H), 1.49 (d, J = 6.4 Hz, 3H). |

### Synthesis of Intermediates L

### Intermediate L1: (trans)-3-{[2-(2,6-dioxopiperidin-3-yl)-1,3-dioxo-2,3-dihydro-1H-isoindol-4-yl]amino}cyclobutane-1-carboxylic acid

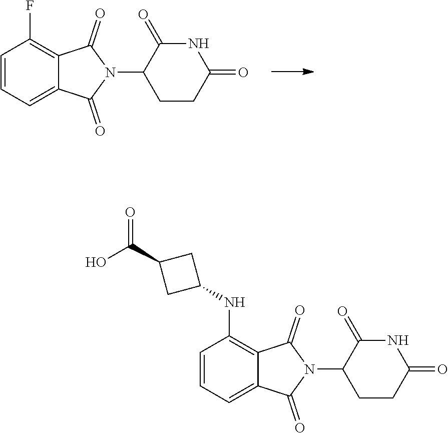 [SMILES: O=C1CCC(N2C(=O)c3cccc(F)c3C2=O)C(=O)N1 ; O=C1CCC(N2C(=O)c3cccc(N[C@H]4C[C@H](C(=O)O)C4)c3C2=O)C(=O)N1]

### Step 1: (trans)-3-{[2-(2,6-dioxopiperidin-3-yl)-1,3-dioxo-2,3-dihydro-1H-isoindol-4-yl]amino}cyclobutane-1-carboxylic acid

A mixture of (trans)-3-aminocyclobutane-1-carboxylic acid (300 mg, 2.60 mmol), 2-(2,6-dioxopiperidin-3-yl)-4-fluoro-2,3-dihydro-1H-isoindole-1,3-dione (598 mg, 2.17 mmol) and DIPEA (1.0 g, 7.80 mmol) in DMSO (15 mL) was stirred at 100° C. under N₂ for 2 h. The mixture was filtered, and the filtrate was diluted with ethyl acetate and water. The layers was separated, and the organic layer was washed with water, dried over Na₂SO₄ and concentrated. The residue was purified by prep-TLC (DCM/MeOH=20/1) to afford the title compound (423 mg, 44%) as a yellow solid. LCMS (Method A2): 2.88 min, m/z:372.4 [M+H]⁺.

The following examples were prepared according to the procedure for the synthesis of Intermediate L1 utilising the indicated amine in Step 1.

TABLE 18
Intermediates L2 - L18

| Intermediate | Name | Structure | LCMS data | SM |
|---|---|---|---|---|
| L2 | 1-[2-(2,6-dioxopiperidin-3- yl)-1,3-dioxo-2,3-dihydro- 1H-isoindol-4-yl]piperidine- 4-carboxylic acid | 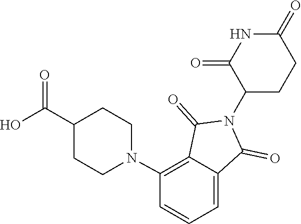 [SMILES: O=C1CCC(N2C(=O)c3cccc(N4CCC(C(=O)O)CC4)c3C2=O)C(=O)N1] | LCMS (Method A2): 2.50 min, m/z: 386 [M + H]⁺ | piperidine-4- carboxylic acid |
| L3 | 2-{1-[2-(2,6-dioxopiperidin- 3-yl)-1,3-dioxo-2,3-dihydro- 1H-isoindol-4-yl]piperidin-4- yl}acetic acid | 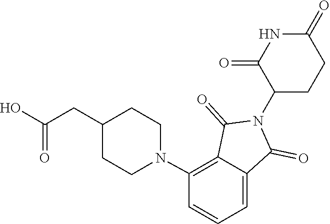 [SMILES: O=C(O)CC1CCN(c2cccc3c2C(=O)N(C2CCC(=O)NC2=O)C3=O)CC1] | LCMS (Method A): 2.70 min, m/z: 400.2 [M + H]⁺ | 2-(piperidin-4- yl)acetic acid |
| L4 | 3-{1-[2-(2,6-dioxopiperidin- 3-yl)-1,3-dioxo-2,3-dihydro- 1H-isoindol-4-yl]piperidin-4- yl}propanoic acid | 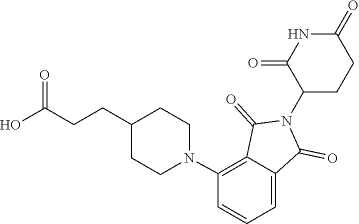 [SMILES: O=C(O)CCC1CCN(c2cccc3c2C(=O)N(C2CCC(=O)NC2=O)C3=O)CC1] | LCMS (Method A2): 3.83 min, m/z: 414.4 [M + H]⁺ | 3-(piperidin-4- yl)propanoic acid |
| L5 | 1-[2-(2,6-dioxopiperidin-3- yl)-1,3-dioxo-2,3-dihydro- 1H-isoindol-4-yl]pyrrolidine- 3-carboxylic acid | 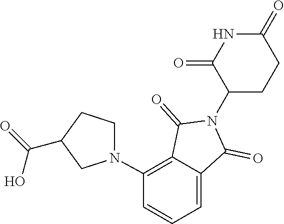 [SMILES: O=C1CCC(N2C(=O)c3cccc(N4CCC(C(=O)O)C4)c3C2=O)C(=O)N1] | LCMS (Method A): 2.58 min, m/z: 372.1 [M + H]⁺. | pyrrolidine-3- carboxylic acid |
| L6 | 2-{1-[2-(2,6-dioxopiperidin- 3-yl)-1,3-dioxo-2,3-dihydro- 1H-isoindol-4-yl]pyrrolidin- 3-yl}acetic acid | 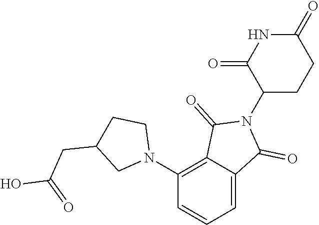 [SMILES: O=C(O)CC1CCN(c2cccc3c2C(=O)N(C2CCC(=O)NC2=O)C3=O)C1] | LCMS (Method A): 2.68 min, m/z: 386.2 [M + H]⁺. | 2-(pyrrolidin-3- yl)acetic acid |
| L7 | (cis)-4-{[2-(2,6- dioxopiperidin-3-yl)-1,3- dioxo-2,3-dihydro-1H- isoindol-4- yl]amino}cyclohexane-1- carboxylic acid | 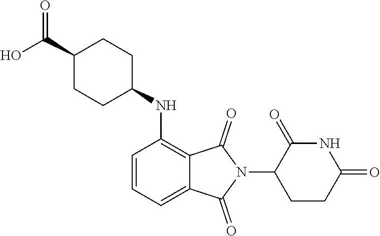 [SMILES: O=C1CCC(N2C(=O)c3cccc(N[C@H]4CC[C@@H](C(=O)O)CC4)c3C2=O)C(=O)N1] | LCMS (Method A): 2.81 min, m/z: 400.1 [M + H]⁺. | (cis)-4- aminocyclohexane- 1-carboxylic acid |
| L8 | (trans)-4-{[2-(2,6- dioxopiperidin-3-yl)-1,3- dioxo-2,3-dihydro-1H- isoindol-4- yl]amino}cyclohexane-1- carboxylic acid | 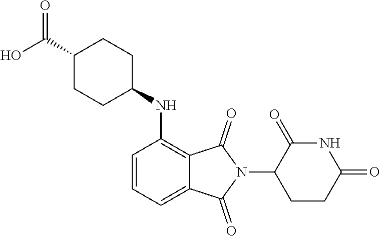 [SMILES: O=C1CCC(N2C(=O)c3cccc(N[C@H]4CC[C@H](C(=O)O)CC4)c3C2=O)C(=O)N1] | LCMS (Method A): 2.77 min, m/z: 400.1 [M + H]⁺. | (trans)-4- aminocyclohexane- 1-carboxylic acid |
| L9 | (cis)-3-{[2-(2,6- dioxopiperidin-3-yl)-1,3- dioxo-2,3-dihydro-1H- isoindol-4- yl]amino}cyclobutane-1- carboxylic acid | 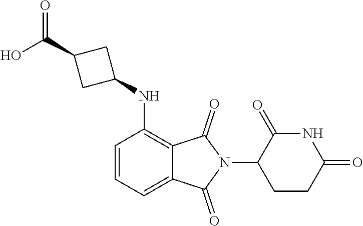 [SMILES: O=C1CCC(N2C(=O)c3cccc(N[C@H]4C[C@@H](C(=O)O)C4)c3C2=O)C(=O)N1] | LCMS (Method A): 2.94 min, m/z: 372.4 [M + H]⁺. | (cis)-3- aminocyclobutane- 1-carboxylic acid |
| L10 | 1-[2-(2,6-dioxopiperidin-3- yl)-1,3-dioxo-2,3-dihydro- 1H-isoindol-4-yl]azetidine-3- carboxylic acid | 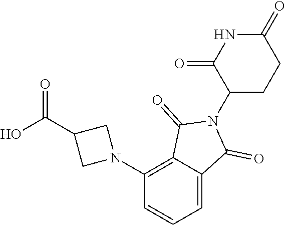 [SMILES: O=C1CCC(N2C(=O)c3cccc(N4CC(C(=O)O)C4)c3C2=O)C(=O)N1] | LCMS (Method A2): 2.46 min, m/z: 358.2 [M + H]⁺. | azetidine-3- carboxylic acid |
| L11 | 1-[2-(2,6-dioxopiperidin-3- yl)-1,3-dioxo-2,3-dihydro- 1H-isoindol-4-yl]piperidine- 3-carboxylic acid | 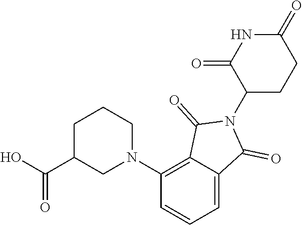 [SMILES: O=C1CCC(N2C(=O)c3cccc(N4CCCC(C(=O)O)C4)c3C2=O)C(=O)N1] | LCMS (Method A): 2.59 min, m/z: 386.1 [M + H]⁺. | piperidine-3- carboxylic acid |
| L12 | 2-{1-[2-(2,6-dioxopiperidin- 3-yl)-1,3-dioxo-2,3-dihydro- 1H-isoindol-4-yl]piperidin-3- yl}acetic acid | 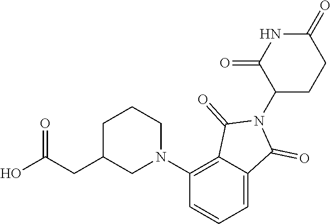 [SMILES: O=C(O)CC1CCCN(c2cccc3c2C(=O)N(C2CCC(=O)NC2=O)C3=O)C1] | LCMS (Method A): 2.78 min, m/z: 400.1 [M + H]⁺. | 2-(piperidin-3- yl)acetic acid |
| L13 | 2-({1-[2-(2,6-dioxopiperidin- 3-yl)-1,3-dioxo-2,3-dihydro- 1H-isoindol-4-yl]piperidin-4- yl}oxy)acetic acid | 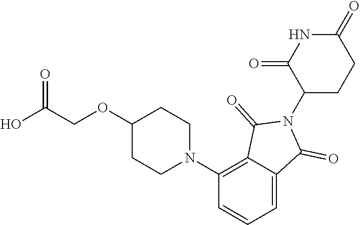 [SMILES: O=C(O)COC1CCN(c2cccc3c2C(=O)N(C2CCC(=O)NC2=O)C3=O)CC1] | LCMS (Method A): 2.41 min, m/z: 416.1 [M + H]⁺. | 2-(piperidin-4- yloxy)acetic acid |
| L14 | 4-[2-(2,6-dioxopiperidin-3- yl)-1,3-dioxo-2,3-dihydro- 1H-isoindol-4- yl]morpholine-2-carboxylic acid | 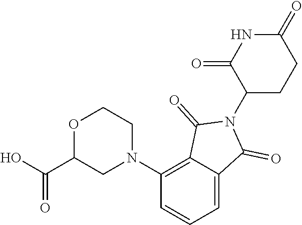 [SMILES: O=C1CCC(N2C(=O)c3cccc(N4CCOC(C(=O)O)C4)c3C2=O)C(=O)N1] | LCMS (Method C): 2.32 min, m/z: 388.5 [M + H]⁺. | morpholine-2- carboxylic acid |
| L15 | 2-{4-[2-(2,6-dioxopiperidin- 3-yl)-1,3-dioxo-2,3-dihydro- 1H-isoindol-4-yl]morpholin- 2-yl}acetic acid | 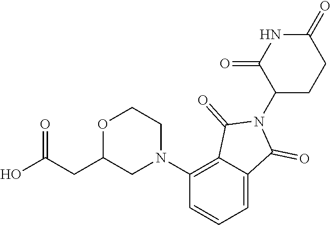 [SMILES: O=C(O)CC1CN(c2cccc3c2C(=O)N(C2CCC(=O)NC2=O)C3=O)CCO1] | LCMS (Method C): 2.64 min, m/z 402.8 [M + H]⁺. | 2-(morpholin-2- yl)acetic acid |
| L16 | 2-[2-(2,6-dioxopiperidin-3- yl)-1,3-dioxo-2,3-dihydro- 1H-isoindol-4-yl]-2- azaspiro[3.3]heptane-6- carboxylic acid | 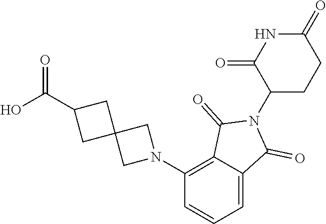 [SMILES: O=C1CCC(N2C(=O)c3cccc(N4CC5(CC(C(=O)O)C5)C4)c3C2=O)C(=O)N1] | LCMS (Method A): 2.16 min, m/z: 397.4 [M + H]⁺. | 2-azaspiro[3.3] heptane-6- carboxylic acid |
| L17 | 4-({[2-(2,6-dioxopiperidin-3- yl)-1,3-dioxo-2,3-dihydro- 1H-isoindol-4- yl]amino}methyl)benzoic acid | 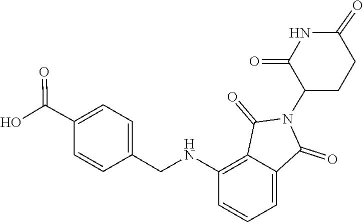 [SMILES: O=C1CCC(N2C(=O)c3cccc(NCc4ccc(C(=O)O)cc4)c3C2=O)C(=O)N1] | LCMS (Method A): 2.66 min, m/z: 408.1 [M + H]⁺. | 4-(aminomethyl) benzoic acid |
| L18 | 2-{1-[2-(2,6-dioxopiperidin- 3-yl)-1,3-dioxo-2,3-dihydro- 1H-isoindol-4-yl]azetidin-3- yl}acetic acid | 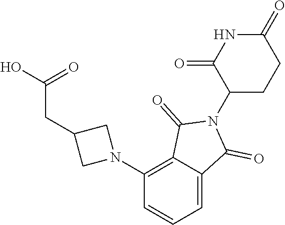 [SMILES: O=C(O)CC1CN(c2cccc3c2C(=O)N(C2CCC(=O)NC2=O)C3=O)C1] | LCMS (method A): 2.56 min, m/z: 372.1 [M + H]⁺ | 2-(azetidin-3- yl)acetic acid |

### Intermediate L19: 7-[2-(2,6-dioxopiperidin-3-yl)-1,3-dioxo-2,3-dihydro-1H-isoindol-4-yl]-7-azaspiro[3.5]nonane-2-carboxylic acid

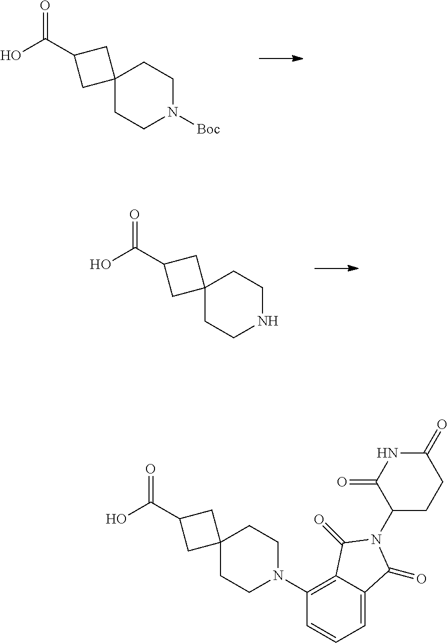 [SMILES: CC(C)(C)OC(=O)N1CCC2(CC1)CC(C(=O)O)C2 ; O=C(O)C1CC2(CCNCC2)C1 ; O=C1CCC(N2C(=O)c3cccc(N4CCC5(CC4)CC(C(=O)O)C5)c3C2=O)C(=O)N1]

### Step 1: 7-azaspiro[3.5]nonane-2-carboxylic acid

To a solution of 7-[(tert-butoxy)carbonyl]-7-azaspiro[3.5]nonane-2-carboxylic acid (1.0 g, 3.71 mmol) in 1,4-dioxane (9 mL) was added 4M HCl/1,4-dioxane (5 mL). The reaction was stirred at RT for 30 min, then concentrated to afford the title compound (600 mg, 96%) as a yellow solid. LCMS (Method A2): 0.44 min, m/z: 170.312, [M+H]⁺.

### Step 2: 7-[2-(2,6-dioxopiperidin-3-yl)-1,3-dioxo-2,3-dihydro-1H-isoindol-4-yl]-7-azaspiro[3.5]nonane-2-carboxylic acid

A mixture of 7-azaspiro[3.5]nonane-2-carboxylic acid (650 mg, 3.84 mmol), 2-(2,6-dioxopiperidin-3-yl)-4-fluoro-2,3-dihydro-1H-isoindole-1,3-dione (1.27 g, 4.60 mmol) and DIPEA (1.48 g, 11.5 mmol) in DMSO (6 mL) was stirred at 100° C. for 2 h. The mixture was purified by Reverse-phase chromatography to afford the title compound (70 mg, 28%) as a yellow solid. LCMS (Method A2): 3.86 min, m/z: 426.4 [M+H]⁺.

### Intermediate L20: 3-{1-[2-(2,6-dioxopiperidin-3-yl)-6-fluoro-1,3-dioxo-2,3-dihydro-1H-isoindol-5-yl]piperidin-4-yl}propanoic acid

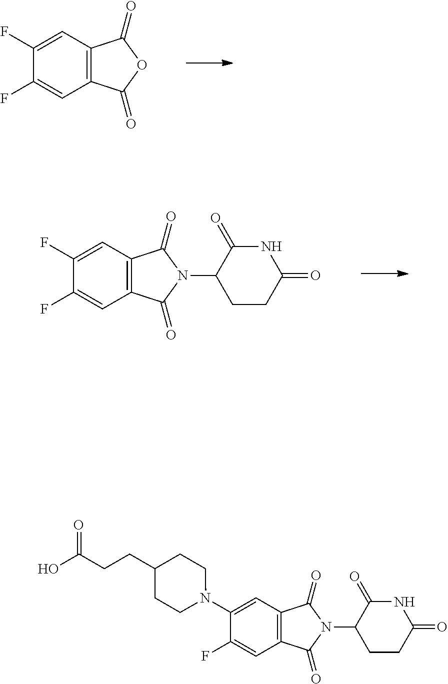 [SMILES: O=C1OC(=O)c2cc(F)c(F)cc21 ; O=C1CCC(N2C(=O)c3cc(F)c(F)cc3C2=O)C(=O)N1 ; O=C(O)CCC1CCN(c2cc3c(cc2F)C(=O)N(C2CCC(=O)NC2=O)C3=O)CC1]

### Step 1: 2-(2,6-dioxopiperidin-3-yl)-5,6-difluoro-2,3-dihydro-1H-isoindole-1,3-dione

A mixture of 5,6-difluoro-1,3-dihydro-2-benzofuran-1,3-dione (1.0 g, 5.43 mmol), 3-aminopiperidine-2,6-dione (894.1 mg, 6.97 mmol) and AcOK (689.8 mg, 7.03 mmol) in AcOH (50 mL) was stirred at 120° C. for 16 h. The mixture was adjusted to pH=8-9 with sat. aq. NaHCO₃, then diluted with water and extracted with EtOAc. The combined organic phases were dried over Na₂SO₄ and concentrated. The residue was purified by column chromatography (PE/EtOAc=2/1) to afford the title compound (353.9 mg, 22%) as a white solid. LCMS (Method A2): 2.73 min., m/z: 295.1 [M+H]⁺.

### Step 2: 3-{1-[2-(2,6-dioxopiperidin-3-yl)-6-fluoro-1,3-dioxo-2,3-dihydro-1H-isoindol-5-yl]piperidin-4-yl}propanoic acid

A mixture of 2-(2,6-dioxopiperidin-3-yl)-5,6-difluoro-2,3-dihydro-1H-isoindole-1,3-dione (200 mg, 679 μmol), 3-(piperidin-4-yl)propanoic acid (118.5 mg, 750 μmol) and DIPEA (351.43 mg, 2.71 mmol) in DMSO (10 mL) was stirred at 100° C. for 16 h. Once cooled, the mixture was diluted with brine and extracted with EtOAc. The combined organics were dried over Na₂SO₄ and concentrated. The residue was purified by prep-TLC (DCM:MeOH, 10:1) to afford the title compound (71.5 mg, 24%) as a yellow solid. LCMS (Method A2): 2.85 min; 432.1 [M+H]⁺.

### Intermediate L21: 3-{4-[2-(2,6-dioxopiperidin-3-yl)-1,3-dioxo-2,3-dihydro-1H-isoindol-4-yl]piperazin-1-yl}-3-oxopropanoic acid

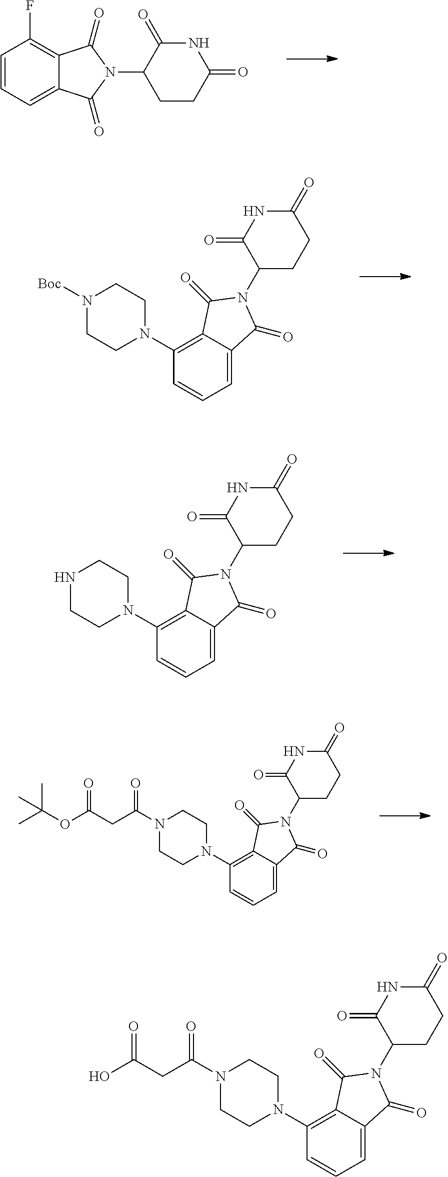 [SMILES: O=C1CCC(N2C(=O)c3cccc(F)c3C2=O)C(=O)N1 ; CC(C)(C)OC(=O)N1CCN(c2cccc3c2C(=O)N(C2CCC(=O)NC2=O)C3=O)CC1 ; O=C1CCC(N2C(=O)c3cccc(N4CCNCC4)c3C2=O)C(=O)N1 ; CC(C)(C)OC(=O)CC(=O)N1CCN(c2cccc3c2C(=O)N(C2CCC(=O)NC2=O)C3=O)CC1 ; O=C(O)CC(=O)N1CCN(c2cccc3c2C(=O)N(C2CCC(=O)NC2=O)C3=O)CC1]

### Step 1: tert-butyl 4-[2-(2,6-dioxopiperidin-3-yl)-1,3-dioxo-2,3-dihydro-1H-isoindol-4-yl]piperazine-1-carboxylate

A mixture of 2-(2,6-dioxopiperidin-3-yl)-4-fluoro-2,3-dihydro-1H-isoindole-1,3-dione (400 mg, 1.44 mmol), tert-butyl piperazine-1-carboxylate (294 mg, 1.58 mmol), and DIPEA (557 mg, 4.31 mmol) in DMSO (10 mL) was stirred at 100° C. under N₂ overnight. The mixture was diluted with H₂O (30 mL) and extracted with EtOAc (3×20 mL). The combined organic layers were dried (Na₂SO₄) and concentrated. The residue was purified by prep-TLC (DCM:MeOH, 30:1) to afford the title compound (305 mg, 48%) as a yellow solid. LCMS (Method A2): 3.36 min, m/z: 343.1 [M-Boc]*.

### Step 2: 2-(2,6-dioxopiperidin-3-yl)-4-(piperazin-1-yl)-2,3-dihydro-1H-isoindole-1,3-dione

A solution of tert-butyl 4-[2-(2,6-dioxopiperidin-3-yl)-1,3-dioxo-2,3-dihydro-1H-isoindol-4-yl]piperazine-1-carboxylate (305 mg, 689 μmol) in 4M HCl/1,4-Dioxane (2 mL) was stireed at RT under N₂ for 1 h. The mixture was diluted with H₂O (100 mL) and extracted with EtOAc (3×100 mL). The combined organic layers were dried (Na₂SO₄) and concentrated. The residue was purified by prep-TLC (DCM:MeOH, 20:1) to afford the title compound (207 mg, 88%) as a yellow solid. LCMS (Method A2): 0.91 min, m/z: 343.2 [M+H]⁺.

### Step 3: tert-butyl 3-{4-[2-(2,6-dioxopiperidin-3-yl)-1,3-dioxo-2,3-dihydro-1H-isoindol-4-yl]piperazin-1-yl}-3-oxopropanoate

A mixture of 2-(2,6-dioxopiperidin-3-yl)-4-(piperazin-1-yl)-2,3-dihydro-1H-isoindole-1,3-dione (130 mg, 379 μmol), 3-(tert-butoxy)-3-oxopropanoic acid (66.6 mg, 416 μmol) HATU (158 mg, 416 μmol) and DIPEA (146 mg, 1.13 mmol) in DMF (5 mL) was stirred at RT under N₂ for 12 h. The mixture was diluted with H₂O (30 mL) and extracted with EtOAc (3×20 mL). The combined organic layers were dried (Na₂SO₄) and concentrated. The residue was purified by prep-TLC (DCM:MeOH, 25:1) to afford the title compound (30.0 mg, 16%) as a yellow solid. LCMS (Method A2): 2.97 min, m/z: 429.1 [M+H]⁺.

### Step 4: 3-{4-[2-(2,6-dioxopiperidin-3-yl)-1,3-dioxo-2,3-dihydro-1H-isoindol-4-yl]piperazin-1-yl}-3-oxopropanoic acid

A solution of tert-butyl 3-(4-{1-[(3Z)-1-(2,6-dioxopiperidin-3-yl)-4-methylidene-2,5-dioxopyrrolidin-3-ylidene]ethyl}piperazin-1-yl)-3-oxopropanoate (30 mg, 63.2 μmol) in 4M HCl/1,4-Dioxane (7 mL) was stirred at RT under N₂ for 1 h. The mixture was diluted with H₂O (30 mL) and extracted with EtOAc (3×20 mL). The combined organic layers were dried (Na₂SO₄) and concentrated. The residue was purified by prep-TLC (DCM:MeOH, 25:1) to afford the title compound (27.0 mg, 47%) as a yellow solid. LCMS (Method A2): 2.65 min, m/z: 429.1 [M+H]⁺.

### Intermediate L22: 3-{1-[2-(1-methyl-2,6-dioxopiperidin-3-yl)-1,3-dioxo-2,3-dihydro-1H-isoindol-4-yl]piperidin-4-yl}propanoic acid

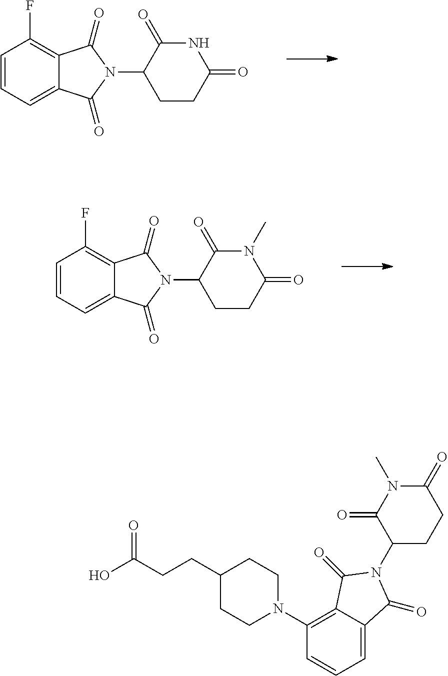 [SMILES: O=C1CCC(N2C(=O)c3cccc(F)c3C2=O)C(=O)N1 ; CN1C(=O)CCC(N2C(=O)c3cccc(F)c3C2=O)C1=O ; CN1C(=O)CCC(N2C(=O)c3cccc(N4CCC(CCC(=O)O)CC4)c3C2=O)C1=O]

### Step 1: 4-fluoro-2-(1-methyl-2,6-dioxopiperidin-3-yl)-2,3-dihydro-1H-isoindole-1,3-dione

A mixture of 2-(2,6-dioxopiperidin-3-yl)-4-fluoro-2,3-dihydro-1H-isoindole-1,3-dione (300 mg, 1.08 mmol), K₂CO₃ (445 mg, 3.23 mmol) and Mel (228 mg, 1.61 mmol) in DMF (3 mL) was stirred at 60° C. for 2 h. The mixture was filtered through Celite and the filter cake was washed with MeOH. The filtrate was concentrated, and the residue was purified by column chromatography (PE:EtOAc, 1:1) to afford the title compound (300 mg, 95%) as a yellow solid. LCMS (Method A): 2.87 min, m/z: 291.3 [M+H]⁺.

### Step 2: 3-{1-[2-(1-methyl-2,6-dioxopiperidin-3-yl)-1,3-dioxo-2,3-dihydro-1H-isoindol-4-yl]piperidin-4-yl}propanoic acid

A mixture of 3-(piperidin-4-yl)propanoic acid (161 mg, 1.02 mmol), 4-fluoro-2-(1-methyl-2,6-dioxopiperidin-3-yl)-2,3-dihydro-1H-isoindole-1,3-dione (266 mg, 918 μmol) and DIPEA (263 mg, 2.04 mmol) in DMSO (4 mL) was stirred at 100° C. for 16 h. The mixture was diluted with water (500 mL) and extracted with EtOAc (3×500 mL). The combined organic phases were washed with water and brine, dried (Na₂SO₄) and concentrated. The residue was purified by column chromatography (PE:EtOAc, 1:1) to afford the title compound (100 mg, 23%) as a yellow solid. LCMS (Method A2): 3.05 min, m/z: 427.5 [M+H]⁺.

### Intermediate L23: 3-{[2-(2,6-dioxopiperidin-3-yl)-1,3-dioxo-2,3-dihydro-1H-isoindol-4-yl]amino}bicyclo[1.1.1]pentane-1-carboxylic acid

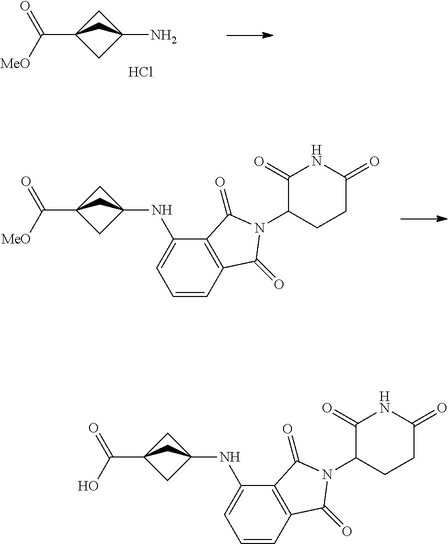 [SMILES: COC(=O)[C@]12C[C@](N)(C1)C2 ; Cl ; COC(=O)[C@]12C[C@](Nc3cccc4c3C(=O)N(C3CCC(=O)NC3=O)C4=O)(C1)C2 ; O=C1CCC(N2C(=O)c3cccc(N[C@]45C[C@](C(=O)O)(C4)C5)c3C2=O)C(=O)N1]

### Step 1: methyl 3-{[2-(2,6-dioxopiperidin-3-yl)-1,3-dioxo-2,3-dihydro-1H-isoindol-4-yl]amino}bicyclo[1.1.1]pentane-1-carboxylate

A mixture of 2-(2,6-dioxopiperidin-3-yl)-4-fluoro-2,3-dihydro-1H-isoindole-1,3-dione (300 mg, 1.08 mmol), methyl 3-aminobicyclo[1.1.1]pentane-1-carboxylate (182 mg, 1.29 mmol) and DIPEA (417 mg, 3.23 mmol) in DMSO (5 mL) was stirred at 100° C. overnight. The reaction mixture was concentrated under reduced pressure, and the residue was diluted with EtOAc and water. The organic layer was separated, washed with water, dried over Na₂SO₄ and concentrated. The residue was purified by prep-TLC (PE/EtOAc=20/1) to affor the title product (356 mg, 83%) as a yellow solid. LCMS (method A): 2.98 min, 398.1 [M+H]⁺.

### Step 2: 3-{[2-(2,6-dioxopiperidin-3-yl)-1,3-dioxo-2,3-dihydro-1H-isoindol-4-yl]amino}bicyclo [1.1.1]pentane-1-carboxylic acid

To a solution of methyl 3-{[2-(2,6-dioxopiperidin-3-yl)-1,3-dioxo-2,3-dihydro-1H-isoindol-4-yl]amino}bicyclo[1.1.1]pentane-1-carboxylate (300 mg, 754 μmol) in 1,4-dioxane (2 mL) aq. HCl (2 mL), and the reaction was stirred at RT overnight. The mixture was concentrated under reduced pressure and the residue was adjusted to pH=6 with sat. aq. Na₂CO₃. The precipitate was collected by filtration, then purified by Prep-TLC (DCM/MeOH=10/1) to afford the title product (266 mg, 92%) as a yellow solid. LCMS (method A): 2.50 min, m/z: 384.1 [M+H]⁺.

### Intermediates L24: 3-{[2-(2,6-dioxopiperidin-3-yl)-1,3-dioxo-2,3-dihydro-1H-isoindol-4-yl]amino}-1-fluorocyclobutane-1-carboxylic acid

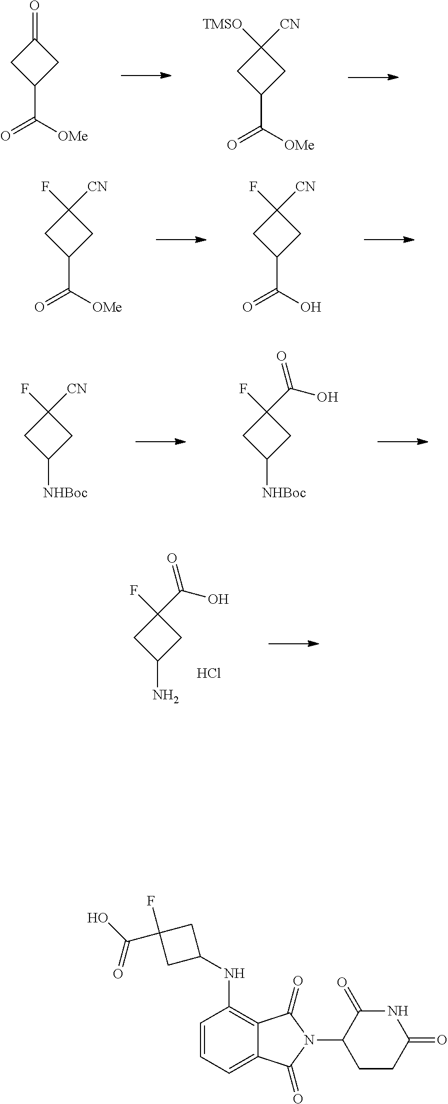

### Step 1: methyl 3-cyano-3-[(trimethylsilyl)oxy]cyclobutane-1-carboxylate

A suspension of methyl 3-oxocyclobutane-1-carboxylate (2.2 g, 17.1 mmol) and Znl₂ (60.0 mg, 188 μmol) was stirred at RT for 10 min, then TMSCN (3.39 g, 34.2 mmol) was added dropwise to maintain the internal temperature below 50° C. The mixture was stirred at RT overnight, then concentrated under reduced pressure to afford the title compound (3.8 g 98%) as a ca. 2:1 mixture of diastereomers. ¹H NMR (400 MHz, CDCl₃): 3.65 (s, 3H), 2.80 (t, 2H), 2.54-2.51 (m, 3H), 0.17 (s, 9H).

### Step 2: methyl 3-cyano-3-fluorocyclobutane-1-carboxylate

To a 0° C. solution of methyl 3-cyano-3-[(trimethylsilyl)oxy]cyclobutane-1-carboxylate (1 g, 4.39 mmol) in DCM was added morph-DAST (1.53 g, 8.78 mmol), and the mixture was stirred at 0° C. for 20 min before conc. HCl (470 mg, 1.65 mmol) was added. The resulting solution was warmed to RT and stirred overnight, then poured into sat. aq. NaHCO₃ (20 mL) and extracted with DCM (70 mL). The organic layer was separated, dried over Na₂SO₄ and concentrated. The residue was purified by column chromatography (PE:EA=10/1) to give the title product (440 mg, 64%) as a brown oil. ¹H NMR (400 MHz, CDCl₃): 3.79 (s, 3H), 3.40 (t, 1H), 3.12-2.98 (m, 2H), 2.90-2.78 (m, 2H).

### Step 3: 3-cyano-3-fluorocyclobutane-1-carboxylic acid

To a solution of methyl 3-cyano-3-fluorocyclobutane-1-carboxylate (9 g, 57.2 mmol) in THE (50 mL) was added a solution of LiOH—H₂O (2.40 g, 57.2 mmol) in H₂O (150 mL) dropwise. The reaction was stirred at RT for 16 h, then diluted with EtOAc (100 mL). The aqueous layer was collected and acidified to pH=2 with 10% aq. NaHSO₄, then extracted with EtOAc (3×150 mL). The combined extracts were dried over Na₂SO₄ and concentrated to give the title product (5 g, 61%) as a yellow oil. ¹H NMR (400 MHz, CDCl₃): 3.47 (t, 1H), 3.12-3.05 (m, 2H), 2.99-2.91 (m, 2H).

### Step 4: tert-butyl N-[3-cyano-3-fluorocyclobutyl]carbamate

To a mixture of 3-cyano-3-fluorocyclobutane-1-carboxylic acid (4 g, 27.9 mmol) and Et₃N (4.22 g, 41.8 mmol) in t-BuOH (35 mL) and MePh (28 mL) was added DPPA (9.95 g, 36.2 mmol) in portions. The reaction was slowly heated to reflux, the stirred overnight. Once cooled, the mixture was poured into sat. aq. NaHCO₃ (200 mL) and extracted with EtOAc (2×30 mL). The combined organic layers were dried over Na₂SO₄ and concentrated. The residue was purified by column chromatography (PE:EtOAc=4:1) to give the title product (1.6 g 27%) as a white solid. ¹H NMR (400 MHz, CDCl₃): 4.85 (bs, 1H), 4.46 (s, 1H), 3.22-3.18 (m, 1H), 3.12-3.01 (m, 1H), 2.78-2.61 (m, 2H), 1.61 (s, 9H).

### Step 5: 3-{[(tert-butoxy)carbonyl]amino}-1-fluorocyclobutane-1-carboxylic acid

A mixture of tert-butyl N-[3-cyano-3-fluorocyclobutyl]carbamate (500 mg, 2.33 mmol) in 1 M NaOH (6 mL) and MeOH (3 mL) was stirred at RT for 48 h. The mixture was concentrated, and the aqueous residue was washed with DCM (20 mL), acidified to pH=2 with 10% aq. NaHSO₄, and extracted with EtOAc (3×20 mL). The combined organic layers were dried over Na₂SO₄ and concentrated to give the title product (290 mg, 53%) as a brown solid. LCMS (Method A): 2.65 min; m/z: 256.1 [M+Na]⁺.

### Step 6: 3-amino-1-fluorocyclobutane-1-carboxylic acid

To a solution of 3-{[(tert-butoxy)carbonyl]amino}-1-fluorocyclobutane-1-carboxylic acid (110 mg, 0.47 mmol) in dioxane (3 mL) was added 4 M HCl in dioxane (3 mL), and the reaction was stirred at RT for 4 h. The mixture was concentracted to give the HCl salt of the title product (60 mg 95%) as a yellow solid.

### Step 7: 3-{[2-(2,6-dioxopiperidin-3-yl)-1,3-dioxo-2,3-dihydro-1H-isoindol-4-yl]amino}-1-fluoro cyclobutane-1-carboxylic acid

To a solution of 3-amino-1-fluorocyclobutane-1-carboxylic acid (150 mg, 1.12 mmol) in DMSO (2 mL) were added DIPEA (602 mg, 4.66 mmol) and 2-(2,6-dioxopiperidin-3-yl)-4-fluoro-2,3-dihydro-1H-isoindole-1,3-dione (257 mg, 0.93 mmol), and the reaction was stirred at 100° C. under N₂ for 16 h. The reaction was concentrated, and the residue was purified by prep-TLC (DCM:MeOH=5/1) to afford the title product (100 mg, 28%) as a yellow solid (ca. 2:1 mixture of diastereomers). LCMS (Method A): 1.72 min; m/z: 390.1 [M+H]⁺.

### Intermediate L25: 2-{1-[2-(2,6-dioxopiperidin-3-yl)-1,3-dioxo-2,3-dihydro-1H-isoindol-4-yl]piperidin-4-yl}cyclopropane-1-carboxylic acid

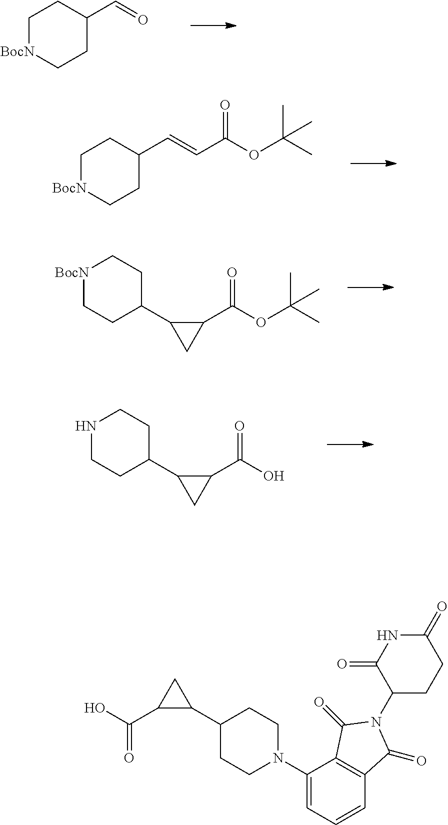 [SMILES: CC(C)(C)OC(=O)N1CCC(C=O)CC1 ; CC(C)(C)OC(=O)/C=C/C1CCN(C(=O)OC(C)(C)C)CC1 ; CC(C)(C)OC(=O)C1CC1C1CCN(C(=O)OC(C)(C)C)CC1 ; O=C(O)C1CC1C1CCNCC1 ; O=C1CCC(N2C(=O)c3cccc(N4CCC(C5CC5C(=O)O)CC4)c3C2=O)C(=O)N1]

### Step 1: tert-butyl 4-[(1E)-3-(tert-butoxy)-3-oxoprop-1-en-1-yl]piperidine-1-carboxylate

To a 0° C. solution of tert-butyl 2-(diethoxyphosphoryl)acetate (5 g, 19.4 mmol) In THE (30 mL) was added MeMgBr (3.53 g, 29.1 mmol) dropwise. The reaction was stirred at 0° C. under N₂ for 30 min before tert-butyl 4-formylpiperidine-1-carboxylate (4.21 g, 19.4 mmol) was added. The mixture was warmed to RT and stirred for 5 h. The mixture was concentrated, and the residue was poured into water (50 mL) and extracted with EtOAc (3×150 mL). The combined organic phases were dried over Na₂SO₄ and concentrated. The residue was purified by column chromatography (PE/EA=200:1-50:1) to give the title product (4.40 g, 73%) as white solid. ¹H NMR (400 MHz, CDCl₃): 6.79 (dd, 1H), 5.72 (dd, 1H), 4.12 (dd, 2H), 2.76 (dt, 2H), 2.28-2.22 (m, 1H), 1.73-1.69 (m, 2H), 1.65 (s, 9H), 1.50 (s, 9H), 1.50-1.32 (m, 2H).

### Step 2: tert-butyl 4-{2-[(tert-butoxy)carbonyl]cyclopropyl}piperidine-1-carboxylate

A mixture of Me₃S(I)O (704 mg, 3.20 mmol) and t-BuOK (538 mg, 4.80 mmol) in DMSO (10 mL) was stirred at RT for 30 min before tert-butyl 4-[(1E)-3-(tert-butoxy)-3-oxoprop-1-en-1-yl]piperidine-1-carboxylate (500 mg, 1.60 mmol) was added. The reaction was stirred at RT under N₂ for 5 h. The mixture was concentrated, then poured into water and extracted with EtOAc. The combined organic phases were dried over Na₂SO₄ and concentrated. The residue was purified by column chromatography (PE:EtOAc=50:1-2:1) to give the title compound (420 mg, 81%) as a yellow oil. LCMS (method A): 2.39 min, m/z: 348.2 [M+Na]⁺.

### Step 3: 2-(piperidin-4-yl)cyclopropane-1-carboxylic acid

To a solution of tert-butyl 4-{2-[(tert-butoxy)carbonyl]cyclopropyl}piperidine-1-carboxylate (500 mg, 1.53 mmol) in dioxane (4 mL) was added 4 M HCl in dioxane (4 mL), and the reaction was stirred at RT for 2 h. The mixture was concentrated under reduced pressure to afford the title product (415 mg, >100%) as a white solid. LCMS (method A): 0.71 min, m/z: 170.1 [M+H]⁺.

### Step 4: 2-{1-[2-(2,6-dioxopiperidin-3-yl)-1,3-dioxo-2,3-dihydro-1H-isoindol-4-yl]piperidin-4-yl}cyclopropane-1-carboxylic acid

To a solution of 2-(piperidin-4-yl)cyclopropane-1-carboxylic acid (415 mg, 2.45 mmol) in dioxane (40 mL) were added DIPEA (1.57 g, 12.2 mmol) and 2-(2,6-dioxopiperidin-3-yl)-4-fluoro-2,3-dihydro-1H-isoindole-1,3-dione (257 mg, 0.93 mmol), and the reaction was stirred at 100° C. under N₂ for 16 h. The reaction was concentrated under reduced pressure and the residue was purified by prep-TLC (DCM:MeOH=5/1) to afford the title product (150 mg, 14%) as a yellow solid. LCMS (Method A): 1.90 min; m/z: 426.10 [M+H]⁺.

### Intermediate L26: (trans)-3-{[2-(2,6-dioxopiperidin-3-yl)-1,3-dioxo-2,3-dihydro-1H-isoindol-4-yl](methyl)amino}cyclobutane-1-carboxylic acid

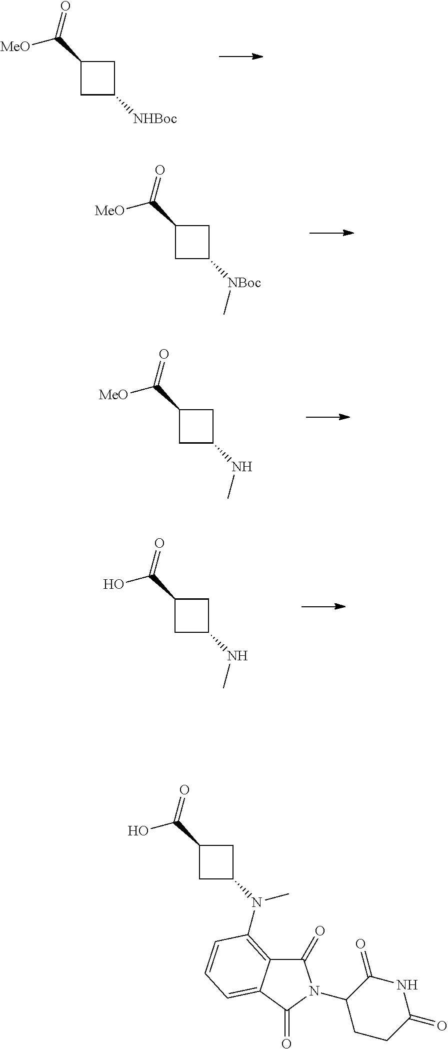

### Step 1: methyl (trans)-3-{[(tert-butoxy)carbonyl](methyl)amino}cyclobutane-1-carboxylate

To a mixture of methyl (trans)-3-{[(tert-butoxy)carbonyl]amino}cyclobutane-1-carboxylate (500 mg, 2.18 mmol) in THE (10 mL) was added NaH (261 mg, 10.9 mmol) in portions. The reaction was stirred at RT until completion (monitored by TLC), then diluted with water (250 mL) and extracted with EtOAc (3×150 mL). The combined organic fractions were washed (brine), dried (MgSO₄) and concentrated to give the title compound (420 mg, 79%) as a white oil.

### Step 2: methyl (trans)-3-(methylamino)cyclobutane-1-carboxylate

A solution of methyl (trans)-3-{[(tert-butoxy)carbonyl](methyl)amino}cyclobutane-1-carboxylate (420 mg, 1.72 mmol) in 4 M HCl/dioxane (20 mL) was stirred at RT for 20 min. The mixture was concentrated to give the title product (210 mg, 86%) as a white solid.

### Step 3: (trans)-3-(methylamino)cyclobutane-1-carboxylic acid

A mixture of methyl (trans)-3-(methylamino)cyclobutane-1-carboxylate (210 mg, 1.46 mmol) and NaOH (116 mg, 2.92 mmol) in 50% aq. MeOH (10 mL) was stirred at 60° C. under N₂ for 2 h. The mixture was concentrated, then the residue was diluted with water and extracted with EtOAc. The combined organic phases were dried over Na₂SO₄ and concentrated to give the title product (120 mg, 64%) as a yellow solid. LCMS (method B): 0.33 min, m/z 129.9 [M+H]⁺.

### Step 4: (trans)-3-{[2-(2,6-dioxopiperidin-3-yl)-1,3-dioxo-2,3-dihydro-1H-isoindol-4-yl](methyl) amino}cyclobutane-1-carboxylic acid

A mixture of (trans)-3-(methylamino)cyclobutane-1-carboxylic acid (100 mg, 774 μmol), DIPEA (299 mg, 2.32 mmol) and 2-(2,6-dioxopiperidin-3-yl)-4-fluoro-2,3-dihydro-1H-isoindole-1,3-dione (257 mg, 0.93 mmol) in DMSO (5 mL) was stirred at 100° C. under N₂ overnight. The mixture was concentrated, then the residue was poured into water and extracted with EtOAc. The combined organic phases were dried over Na₂SO₄ and concentrated. The residue was purified by Prep-TLC (DCM/MeOH=20/1) to give the title product (50.0 mg, 22%) as a yellow solid. LCMS (method B): 1.65 min, m/z 386.1 [M+H]⁺.

### Intermediate L27: (trans)-3-{[2-(2,6-dioxopiperidin-3-yl)-1,3-dioxo-2,3-dihydro-1H-isoindol-5-yl]amino}cyclobutane-1-carboxylic acid

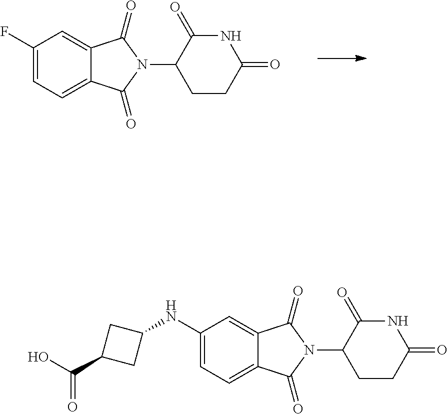 [SMILES: O=C1CCC(N2C(=O)c3ccc(F)cc3C2=O)C(=O)N1 ; O=C1CCC(N2C(=O)c3ccc(N[C@H]4C[C@H](C(=O)O)C4)cc3C2=O)C(=O)N1]

### Step 1: (trans)-3-{[2-(2,6-dioxopiperidin-3-yl)-1,3-dioxo-2,3-dihydro-1H-isoindol-5-yl]amino}cyclobutane-1-carboxylic acid

A mixture of (trans)-3-aminocyclobutane-1-carboxylic acid (100 mg, 868 μmol) 2-(2,6-dioxopiperidin-3-yl)-5-fluoro-2,3-dihydro-1H-isoindole-1,3-dione (239 mg, 868 μmol) and DIPEA (336 mg, 2.60 mmol) in DMSO (5 mL) was stirred at 100° C. under N₂ for 2 h. The mixture was filtered, and the filtrate was diluted with ethyl acetate and water. The layers were separated, and the organic layer was washed with water, dried over Na₂SO₄ and concentrated. The residue was purified by prep-TLC (DCM/MeOH=20/1) to afford the title compound (70.0 mg, 22%) as a yellow solid. LCMS (Method C): 2.75 min. m/z: 372.7 [M+H]⁺.

### Intermediate L28: (trans)-3-{[2-(2,6-dioxopiperidin-3-yl)-1,3-dioxo-2,3-dihydro-1H-isoindol-5-yl](methyl)amino}cyclobutane-1-carboxylic acid

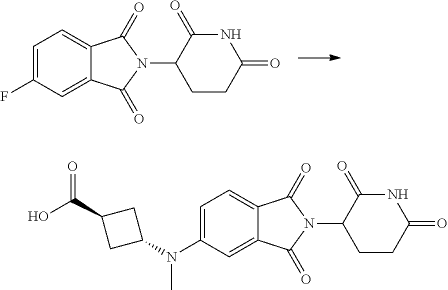 [SMILES: O=C1CCC(N2C(=O)c3ccc(F)cc3C2=O)C(=O)N1 ; CN(c1ccc2c(c1)C(=O)N(C1CCC(=O)NC1=O)C2=O)[C@H]1C[C@H](C(=O)O)C1]

### Step 1: (trans)-3-{[2-(2,6-dioxopiperidin-3-yl)-1,3-dioxo-2,3-dihydro-1H-isoindol-5-yl](methyl) amino}cyclobutane-1-carboxylic acid

The title compound was prepared analogously to Intermediate L27 using (trans)-3-(methylamino) cyclobutane-1-carboxylic acid. The compound was obtained as a yellow solid (100 mg, 22%). LCMS (method B): 1.60 min, m/z 386.1 [M+H]⁺.

### Intermediate L29: 4-[2-(2,6-dioxopiperidin-3-yl)-1,3-dioxo-2,3-dihydro-1H-isoindol-4-yl]cyclohexane-1-carboxylic acid

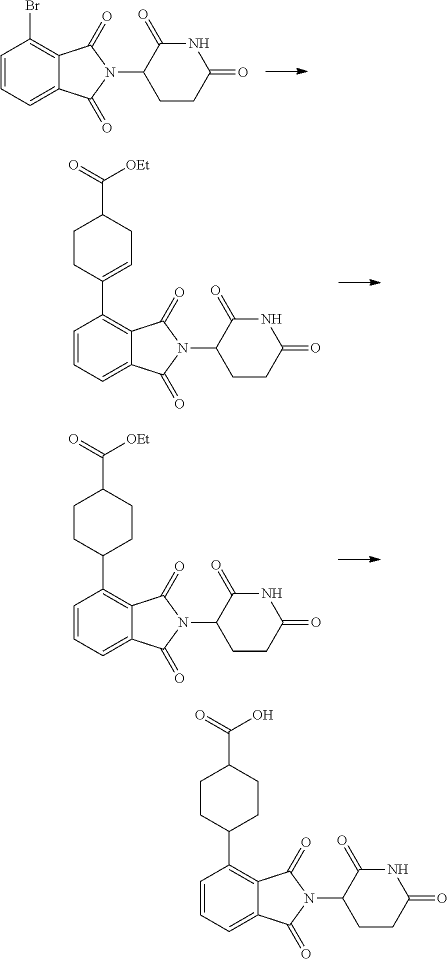

### Step 1: ethyl 4-[2-(2,6-dioxopiperidin-3-yl)-1,3-dioxo-2,3-dihydro-1H-isoindol-4-yl]cyclohex-3-ene-1-carboxylate

A mixture of 4-bromo-2-(2,6-dioxopiperidin-3-yl)-2,3-dihydro-1H-isoindole-1,3-dione (500 mg, 1.48 mmol), ethyl 4-(4,4,5,5-tetramethyl-1,3,2-dioxaborolan-2-yl)cyclohex-3-ene-1-carboxylate (495 mg, 1.77 mmol), Pd(dppf)Cl₂ (108 mg, 148 μmol) and Na₂CO₃ (469 mg, 4.43 mmol) in 1,4-Dioxane:H₂O (5:1, 20 mL) was stirred at 100° C. for 3 h. The mixture was concentrated and the residue was adjusted to pH=7 with dilute HCl, then diluted with H₂O (80 mL) and extracted with EtOAc (3×80 mL). The combined organics were concentrated under reduced pressure and the residue was purified by Prep-TLC (DCM/MeOH=30/1) to give the title product (324 mg, 53%) as a yellow solid. LCMS (method B): 2.27 min, m/z 411.2 [M+H]⁺.

### Step 2: ethyl 4-[2-(2,6-dioxopiperidin-3-yl)-1,3-dioxo-2,3-dihydro-1H-isoindol-4-yl]cyclohexane-1-carboxylate

A mixture of ethyl 4-[2-(2,6-dioxopiperidin-3-yl)-1,3-dioxo-2,3-dihydro-1H-isoindol-4-yl]cyclohex-3-ene-1-carboxylate (324 mg, 789 μmol) and Raney-Ni (463 mg, 7.89 mmol) in EtOAc (15 mL) was stirred at RT under a H₂ atmosphere (5 MPa) overnight. The mixture was concentrated and the residue was purified by Prep-TLC (DCM/MeOH=30/1) to afford the title product (169 mg, 52%) as a yellow solid. LCMS (method B): 2.44 min, m/z 413.2 [M+H]⁺.

### Step 3: 4-[2-(2,6-dioxopiperidin-3-yl)-1,3-dioxo-2,3-dihydro-1H-isoindol-4-yl]cyclohexane-1-carboxylic acid

A solution of ethyl 4-[2-(2,6-dioxopiperidin-3-yl)-1,3-dioxo-2,3-dihydro-1H-isoindol-4-yl]cyclohexane-1-carboxylate (169 mg, 409 μmol) in 4M HCl/Dioxane (10 mL) was stirred at 50° C. for 2 h. The mixture was concentrated and the residue was purified by Prep-TLC (DCM/MeOH=20/1) to give the title product (20 mg, 13%) as a white solid. LCMS (method B): 1.75 min, m/z 407.1 [M+Na]⁺.

### Intermediate L30: 4-[2-(2,6-dioxopiperidin-3-yl)-1,3-dioxo-2,3-dihydro-1H-isoindol-5-yl]cyclohexane-1-carboxylic acid

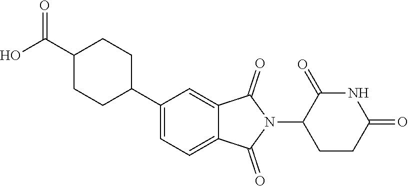 [SMILES: O=C1CCC(N2C(=O)c3ccc(C4CCC(C(=O)O)CC4)cc3C2=O)C(=O)N1]

The title compound was prepared from 2-(2,6-dioxopiperidin-3-yl)-5-bromo-2,3-dihydro-1H-isoindole-1,3-dione according to the procedure for the synthesis of Intermediate L29. The title compound was obtained as a white solid (120 mg, 86%). LCMS (Method B): 1.79 min, m/z: 385.2 [M+H]⁺.

### Intermediate L31: N-(4-{4-amino-7-[1-(1-{2-[2-(2,6-dioxopiperidin-3-yl)-1,3-dioxo-2,3-dihydro-1H-isoindol-4-yl]acetyl}piperidin-4-yl)-1H-pyrazol-4-yl]-1-methyl-1H-pyrazolo[4,3-c]pyridin-3-yl}-2-[(1 S)-1-(4-fluorophenyl)ethoxy]phenyl)-1,1-difluoromethanesulfonamide

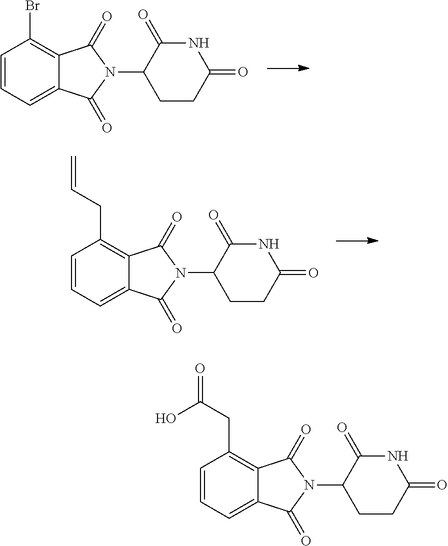

### Step 1: 2-(2,6-dioxopiperidin-3-yl)-4-(prop-2-en-1-yl)-2,3-dihydro-1H-isoindole-1,3-dione

To a solution of 4-bromo-2-(2,6-dioxopiperidin-3-yl)-2,3-dihydro-1H-isoindole-1,3-dione (400 mg, 1.18 mmol) in 1,4-Dioxane/H₂O (4/1, 10 mL) were added 4,4,5,5-tetramethyl-2-(prop-2-en-1-yl)-1,3,2-dioxaborolane (594 mg, 3.54 mmol), Pd(dppf)Cl₂ (87 mg, 118 μmol) and K₂CO₃ (490 mg, 3.54 mmol), and the reaction was stirred at 100° C. under N₂ overnight. The mixture was diluted with water and extracted with EtOAc. The organic layer was collected, washed with brine and concentrated. The residue was purified by column chromatography (DCM:MeOH=30:1) to afford the title product (200 mg, 57%) as a brown solid. LCMS (method B): 2.09 min, m/z: 299 [M+H]⁺.

### Step 2: 2-[2-(2,6-dioxopiperidin-3-yl)-1,3-dioxo-2,3-dihydro-1H-isoindol-4-yl]acetic acid

To a solution of 2-(2,6-dioxopiperidin-3-yl)-4-(prop-2-en-1-yl)-2,3-dihydro-1H-isoindole-1,3-dione (100 mg, 335 μmol) in a mixture of CCl₄ (0.5 mL), H₂O (0.5 mL) and MeCN (1 mL) were added RuCl₃ (7 mg, 33.7 μmol) and NaIO₄ (359 mg, 1.67 mmol), and the mixture was stirred at 40° C. under N₂ overnight. The mixture was filtered through Celite and the filter cake was washed with EtOAc. The filtrate was concentrated and the residue was purified by column chromatography (DCM:MeOH=40:1) to afford the title product (80.0 mg, 76%) as a brown solid. LCMS (method B): 1.21 min; m/z: 317.1 [M+H]⁺.

### Intermediate L32: 2-[2-(2,6-dioxopiperidin-3-yl)-1,3-dioxo-2,3-dihydro-1H-isoindol-5-yl]acetic acid

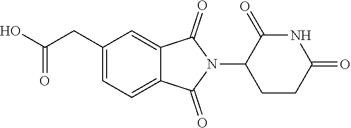 [SMILES: O=C(O)Cc1ccc2c(c1)C(=O)N(C1CCC(=O)NC1=O)C2=O]

The title compound was prepared from 2-(2,6-dioxopiperidin-3-yl)-5-bromo-2,3-dihydro-1H-isoindole-1,3-dione according to the procedure for the synthesis of Intermediate L31. The title compound was obtained as a yellow oil (130 mg, 73%). LCMS (Method B): 1.11 min, m/z: 317.1 [M+H]⁺.

### Compound 1047: N-{4-[4-amino-1-methyl-7-(1-{1-[(trans)-3-{[2-(2,6-dioxopiperidin-3-yl)-1,3-dioxo-2,3-dihydro-1H-isoindol-4-yl]amino}cyclobutanecarbonyl]piperidin-4-yl}-1H-pyrazol-4-yl)-1H-pyrazolo[4,3-c]pyridin-3-yl]-2-[(1 S)-1-(4-fluorophenyl)ethoxy]phenyl}-1,1-difluoromethanesulfonamide

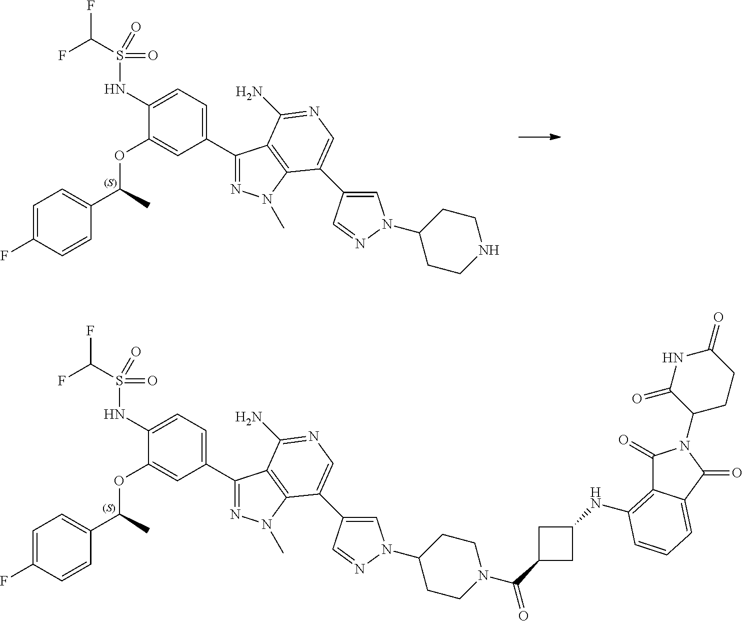

### Step 1: N-{4-[4-amino-1-methyl-7-(1-{1-[(trans)-3-{[2-(2,6-dioxopiperidin-3-yl}-1,3-dioxo-2,3-dihydro-1H-isoindol-4-yl]amino}cyclobutanecarbonyl]piperidin-4-yl-1H-pyrazol-4-yl)-1H-pyrazolo[4,3-c]pyridin-3-yl]-2-[(1 S)-1-(4-fluorophenyl)ethoxy]phenyl}-1,1-difluoromethanesulfonamide

A mixture of (trans)-3-{[2-(2,6-dioxopiperidin-3-yl)-1,3-dioxo-2,3-dihydro-1H-isoindol-4-yl]amino}cyclobutane-1-carboxylic acid (400 mg, 1.07 mmol), N-(4-{4-amino-1-methyl-7-[1-(piperidin-4-yl)-1H-pyrazol-4-yl]-1H-pyrazolo[4,3-c]pyridin-3-yl}-2-[(1S)-1-(4-fluorophenyl)ethoxy]phenyl)-1,1-difluoromethanesulfonamide (685 mg, 1.07 mmol), HATU (407 mg, 1.07 mmol) and DIPEA (138 mg, 1.07 mmol) in DMF (10 mL) was stirred at RT for 2 h. The mixture was filtered, then the filtrate was diluted with EtOAc and water. The layers were separated, and the organic layer was washed with water, dried over Na₂SO₄ and concentrated. The residue was purified by prep-TLC (DCM/MeOH=20/1) to afford the title compound (85.0 mg, 8%) as a yellow solid. LCMS (Method A2): 3.17 min, m/z: 497.8 [M+2H]²⁺. ¹H NMR (400 MHz, DMSO-d₆): 11.11 (s, 1H), 8.01 (s, 1H), 7.60 (d, J=4.9 Hz, 2H), 7.58-7.50 (m, 3H), 7.38 (d, J=8.1 Hz, 1H), 7.15 (t, J=8.8 Hz, 2H), 7.11-6.97 (m, 3H), 6.92 (d, J=8.6 Hz, 1H), 6.59 (d, J=6.2 Hz, 1H), 5.65 (d, J=6.4 Hz, 3H), 5.07 (dd, J=12.9, 5.4 Hz, 1H), 4.50 (q, J=11.5, 10.5 Hz, 2H), 4.09 (q, J=6.8 Hz, 1H), 3.80 (d, J=13.2 Hz, 1H), 3.68 (s, 3H), 3.48 (t, J=5.1 Hz, 1H), 3.16 (t, J=12.8 Hz, 1H), 2.96-2.77 (m, 2H), 2.68 (s, 2H), 2.25 (s, 2H), 2.11 (d, J=12.4 Hz, 2H), 1.94-1.76 (m, 2H), 1.55 (d, J=6.3 Hz, 3H), 1.43 (s, 1H), 1.24 (s, 3H).

The following examples were prepared according to the procedure for the synthesis of Compound 1047 utilising the indicated Intermediate in Step 1.

TABLE 19
Compounds 1044, 1048, 1043, 1054, 1053, 1071, 1058, 1055, 1051, 1077, 1073, 1072, 1070, 1081, 1074, 1093,
1112, 1069, 1087, 1098, 1141, 1118, 1110, 1111, 1129, 1130, 1119, 1094, 1106, 1125, 1126, 1137 and 1142

| Exam- | | |
|---|---|---|
| ple | Structure | Compound Name and Analytical Data |
| 1044 | 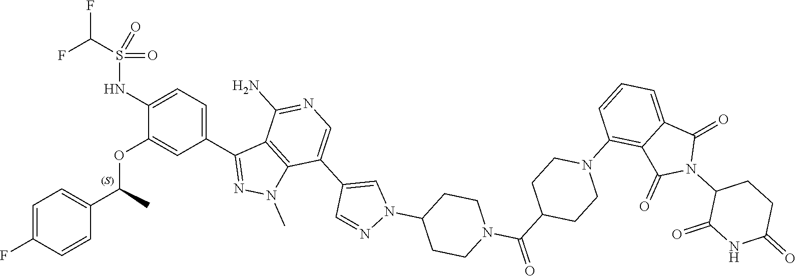 | N-(4-{4-amino-7-[1-(1-{1-[2-(2,6-dioxopiperidin-3-yl)- 1,3-dioxo-2,3-dihydro-1H-isoindol-4-yl]piperidine-4-carbonyl}piperidin-4-yl)-1H-pyrazol-4-yl]-1-methyl-1H-pyrazolo [4,3-c]pyridin-3-yl}-2-[(1S)-1-(4-fluorophenyl)ethoxy]phenyl)-1, 1-difluoromethanesulfonamide From Intermediate L2. LCMS (Method A2): 5.42 min, m/z: 1100.5 [M + H]⁺. ¹H NMR (400 MHZ, DMSO-d₆): 11.08 (s, 1H), 10.59 (s, 1H), 8.16 (s, 1H), 7.73 (s, 1H), 7.69 (dd, J = 8.5, 7.1 Hz, 1H), 7.63 (s, 1H), 7.60-7.56 (m, 2H), 7.44 (d, J = 8.1 Hz, 1H), 7.34 (dd, J = 7.8, 5.8 Hz, 2H), 7.22-7.15 (m, 4H), 7.13 (d, J = 1.8 Hz, 1H), 6.97 (d, J = 52.6 Hz, 1H), 5.65 (d, J = 6.4 Hz, 1H), 5.09 (dd, J = 12.9, 5.4 Hz, 1H), 4.54 (d, J = 12.2 Hz, 2H), 4.17 (d, J = 13.5 Hz, 1H), 3.75 (s, 5H), 3.06-2.74 (m, 6H), 2.65-2.53 (m, 2H), 2.27-2.07 (m, 4H), 2.02 (dq, J = 14.2, 6.4 Hz, 3H), 1.78 (s, 5H), 1.59 (d, J = 6.3 Hz, 3H). |
| 1048 | 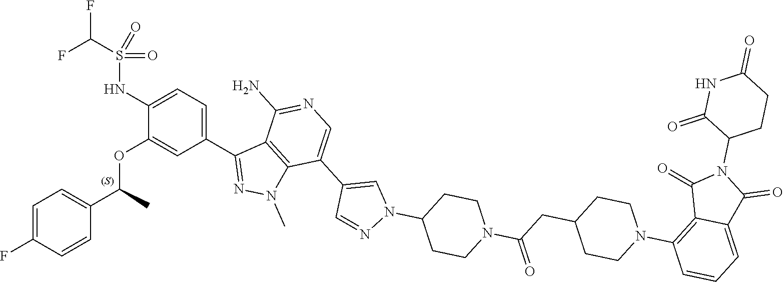 | N-[4-(4-amino-7-{1-[1-(2-{1-[2-(2,6-dioxopiperidin-3-yl)- 1,3-dioxo-2,3-dihydro-1H-isoindol-4-yl]piperidin-4- yl}acetyl)piperidin-4-yl]-1H-pyrazol-4-yl}-1-methyl-1H- pyrazolo[4,3-c]pyridin-3-yl)-2-[(1S)-1- (4-fluorophenyl)ethoxy]phenyl]- 1,1-difluoromethanesulfonamide From Intermediate L3. LCMS (Method A1): 2.93 min, m/z: 511.8 [M + 2H]²⁺. ¹H NMR (400 MHZ, CDCl₃): 11.08 (s, 1H), 10.58 (s, 1H), 8.14 (s, 1H), 7.73 (s, 1H), 7.68 (t, J = 8 Hz, 1H), 7.63 (s, 1H), 7.60-7.56 (m, 2H), 7.49 (s, 1H), 7.44 (d, J = 8 Hz, 1H), 7.33 (t, J = 8 Hz, 2H), 7.20-7.13 (m, 4H), 7.04 (t, J = 52 Hz, 1H), 5.64 (q, J = 8 Hz, 1H), 5.08 (dd, J = 8 Hz, 12 Hz, 1H), 4.54 (s, 2H), 4.07 (d, J = 12 Hz, 1H), 3.75 (s, 3H), 3.68 (d, J = 12 Hz, 3H), 2.92-2.76 (m, 5H), 2.61-2.57 (m, 2H), 2.39-2.33 (m, 2H), 2.25-1.91 (m, 9H), 1.81 (d, J = 12 Hz, 3H), 1.59 (d, J = 8 Hz, 3H), 1.47-1.41 (m, 2H). |
| 1043 | 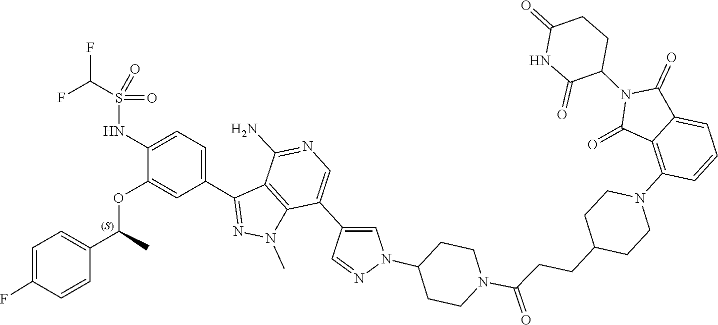 | N-[4-(4-amino-7-{1-[1-(3-{1-[2-(2,6-dioxopiperidin-3-yl)- 1,3-dioxo-2,3-dihydro-1H-isoindol-4-yl]piperidin-4- yl}propanoyl)piperidin-4-yl]-1H-pyrazol-4-yl}-1-methyl-1H- pyrazolo[4,3-c]pyridin-3-yl)-2-[(1S)-1-(4-fluorophenyl)ethoxy] phenyl]-1,1-difluoromethanesulfonamide From Intermediate L4. LCMS (Method A2): 2.94 min, m/z: 1036.1 [M + H]⁺. ¹H NMR (400 MHZ, DMSO-d₆): 11.09 (s, 1H), 8.03 (s, 1H), 7.68 (dd, J = 8.5, 7.1 Hz, 1H), 7.63 (s, 1H), 7.57 (tt, J = 6.2, 2.6 Hz, 3H), 7.41 (d, J = 8.0 Hz, 1H), 7.33 (t, J = 7.9 Hz, 2H), 7.21-7.09 (m, 4H), 6.94 (d, J = 52.6 Hz, 1H), 5.66 (q, J = 6.3 Hz, 1H), 5.09 (dd, J = 12.8, 5.4 Hz, 1H), 4.51 (d, J = 12.6 Hz, 2H), 4.04 (d, J = 7.3 Hz, 1H), 3.70 (s, 4H), 3.23 (t, J = 13.0 Hz, 2H), 2.94-2.67 (m, 4H), 2.65-2.54 (m, 1H), 2.47-2.31 (m, 2H), 2.24-1.89 (m, 4H), 1.82 (d, J = 12.1 Hz, 3H), 1.59 (d, J = 6.2 Hz, 3H), 1.52 (d, J = 6.8 Hz, 2H), 1.36 (d, J = 11.4 Hz, 2H), 0.87 (t, J = 6.7 Hz, 1H). |
| 1054 | 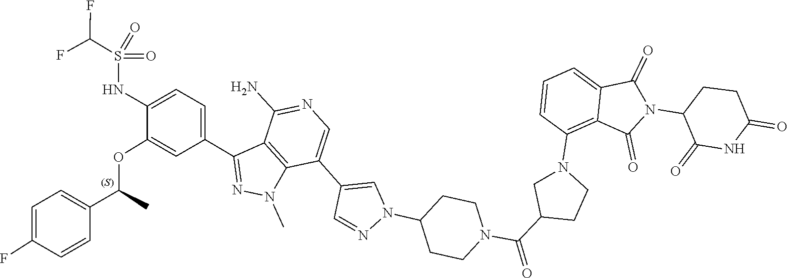 | N-(4-{4-amino-7-[1-(1-{1-[2-(2,6-dioxopiperidin-3-yl)- 1,3-dioxo-2,3-dihydro-1H-isoindol-4-yl]pyrrolidine-3-carbonyl}piperidin-4-yl)-1H-pyrazol-4-yl]-1-methyl-1H-pyrazolo [4,3-c]pyridin-3-yl}-2-[(1S)-1-(4-fluorophenyl)ethoxy]phenyl)- 1,1-difluoromethanesulfonamide From Intermediate L5. LCMS (Method A1): 2.81 min, m/z: 497.8 [M + 2H]²⁺. ¹H NMR (400 MHZ, DMSO-d₆): 11.06 (s, 1H), 8.03 (s, 1H), 7.56-7.40 (m, 6H), 7.17-7.02 (m, 7H), 5.93 (s, 1H), 6.65 (d, J = 6.8 Hz, 1H), 5.06 (d, J = 8.8 Hz, 1H), 4.51 (q, J = 13.2 Hz, 2H), 4.16 (d, J = 13.6 Hz, 1H), 3.76-3.57 (m, 9H), 2.86-2.82 (m, 2H), 2.22-1.84 (m, 8H), 1.58 (d, J = 6.4 Hz, 3H). |
| 1053 | 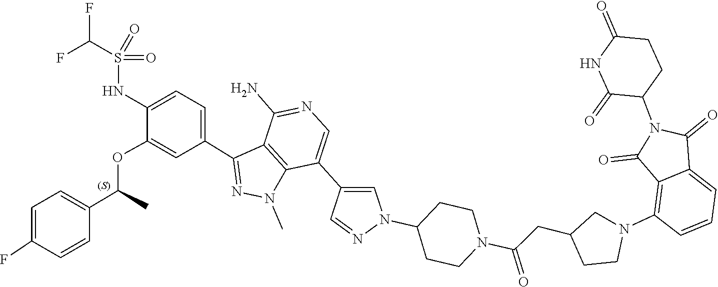 | N-[4-(4-amino-7-{1-[1-(2-{1-[2-(2,6-dioxopiperidin-3-yl)- 1,3-dioxo-2,3-dihydro-1H-isoindol-4-yl]pyrrolidin-3-yl}acetyl)piperidin-4-yl]-1H-pyrazol-4-yl}-1-methyl-1H-pyrazolo [4,3-c]pyridin-3-yl)-2-[(1S)-1-(4-fluorophenyl)ethoxy]phenyl]-1,1- difluoromethanesulfonamide From Intermediate L6. LCMS (Method A1): 2.96 min, m/z: 504.8 [M + 2H]²⁺. ¹H NMR (400 MHZ, DMSO-d₆): 11.05 (s, 1H), 8.14 (s, 1H), 8.00 (s, 1H), 7.61 (s, 1H), 7.55 (dd, J = 8.3, 5.4 Hz, 4H), 7.39 (d, J = 8.0 Hz, 1H), 7.16 (t, J = 8.8 Hz, 2H), 7.13-7.05 (m, 4H), 7.03-6.74 (m, 1H), 5.65 (dd, J = 12.4, 6.0 Hz, 3H), 5.06 (dd, J = 12.8, 5.4 Hz, 1H), 4.49 (t, J = 12.9 Hz, 2H), 4.02 (d, J = 12.0 Hz, 1H), 3.68 (s, 3H), 3.61-3.41 (m, 3H), 2.95-2.72 (m, 2H), 2.58 (s, 2H), 2.19-2.05 (m, 3H), 2.02-1.78 (m, 3H), 1.68 (s, 1H), 1.57 (d, J = 6.3 Hz, 3H), 1.35-1.19 (m, 2H). |
| 1071 | 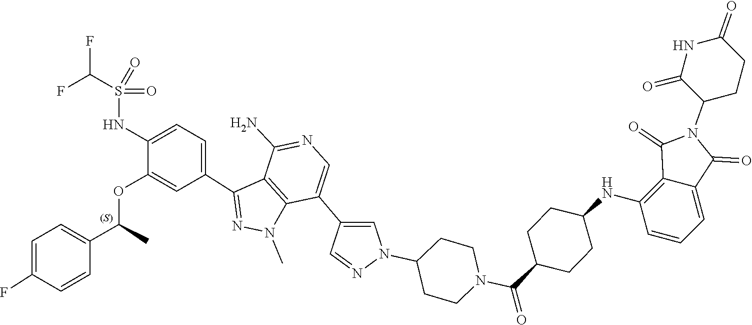 | N-{4-[4-amino-1-methyl-7-(1-{1-[(cis)-4-{[2-(2,6-dioxopiperidin- 3-yl)-1,3-dioxo-2,3-dihydro-1H-isoindol-4-yl]amino}cyclohexane-carbonyl]piperidin-4-yl}-1H- pyrazol-4-yl)-cyclohexanecarbonyl]piperidin-4-yl}-1H- pyrazol-4-yl)-1H-pyrazolo[4,3-c]pyridin-3-yl]-2- [(1S)-1-(4-fluorophenyl)ethoxy]phenyl}-1,1- difluoromethanesulfonamide From Intermediate L7. LCMS: (Method A): 2.96 min, m/z: 1022.3 [M + H]⁺. ¹H NMR (400 MHZ, DMSO-d₆): 12.65 (s, 2H), 11.10 (s, 1H), 10.55 (s, 1H), 8.13 (d, J = 1.4 Hz, 1H), 7.71 (s, 1H), 7.60 (d, J = 14.1 Hz, 3H), 7.44 (d, J = 8.1 Hz, 1H), 7.24-7.11 (m, 5H), 7.04 (d, J = 1.8 Hz, 2H), 6.53 (s, 1H), 5.64 (d, J = 6.4 Hz, 1H), 5.06 (s, 1H), 4.53 (s, 2H), 4.11 (d, J = 12.3 Hz, 1H), 3.74 (s, 2H), 2.83 (s, 3H), 2.14 (s, 2H), 1.99 (s, 2H), 1.80 (s, 4H), 1.59 (d, J = 6.3 Hz, 3H). |
| 1058 | 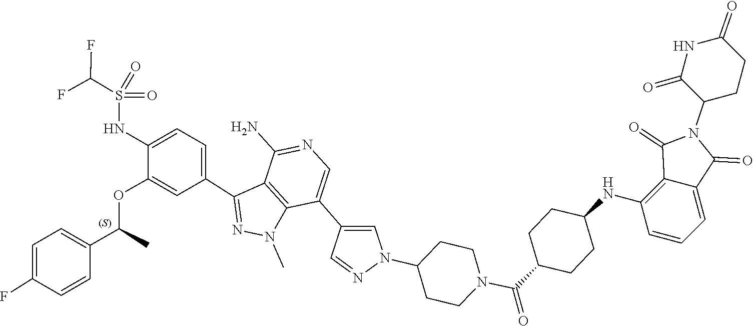 | N-{4-[4-amino-1-methyl-7-(1-{1-[(trans)-4-{[2-(2,6- dioxopiperidin-3-yl)-1,3-dioxo-2,3-dihydro-1H-isoindol-4-yl]amino}cyclohexane-carbonyl]piperidin-4-yl}- 1H-pyrazol-4-yl)-1H-pyrazolo[4,3-c]pyridin-3- yl]-2-[(1S)-1-(4-fluorophenyl)ethoxy]phenyl}-1,1- difluoromethanesulfonamide From Intermediate L8. LCMS: (Method A): 2.87 min, m/z: 1022.3 [M + H]⁺. ¹H NMR (400 MHZ, DMSO-d₆): 11.09 (s, 1H), 8.04 (s, 1H), 7.63 (s, 1H), 7.57 (d, J = 2.6 Hz, 2H), 7.41 (d, J = 8.0 Hz, 1H), 7.37-7.07 (m, 7H), 7.05-7.02 (m, 1H), 6.16 (d, J = 8.3 Hz, 1H), 5.65 (q, J = 6.3 Hz, 1H), 5.05 (dd, J = 12.8, 5.4 Hz, 1H), 4.67-4.16 (m, 3H), 3.70 (s, 3H), 2.93-2.86 (m, 1H), 2.81-2.54 (m, 4H), 2.19-1.97 (m, 6H), 1.75 (dd, J = 13.1, 6.9 Hz, 3H), 1.58 (d, J = 6.3 Hz, 4H), 1.56-1.18 (m, 8H), 0.87 (t, J = 7.4 Hz, 1H). |
| 1055 | 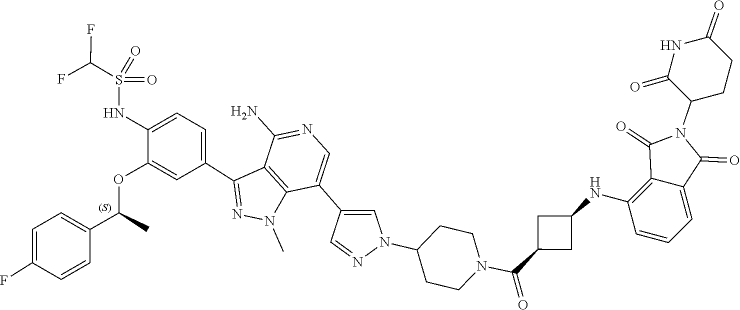 | N-{4-[4-amino-1-methyl-7-(1-{1-[(cis)-3-{[2-(2,6- dioxopiperidin-3-yl)-1,3-dioxo-2,3-dihydro-1H- isoindol-4-yl]amino}cyclobutane-carbonyl]piperidin- 4-yl}-1H-pyrazol-4-yl)-1H-pyrazolo[4,3-c]pyridin- 3-yl]-2-[(1S)-1-(4-fluorophenyl)ethoxy]phenyl}- 1,1-difluoromethanesulfonamide From Intermediate L9. LCMS (Method A1): 2.84 min, m/z: 497.8 [M + 2H]²⁺. ¹H NMR (400 MHZ, DMSO-d₆): 11.10 (s, 1H), 8.07 (s, 1H), 7.62-7.55 (m, 5H), 7.41 (d, J = 8.0 Hz, 1H), 7.19-7.02 (m, 7H), 6.45 (d, J = 8.0 Hz, 1H), 5.95 (s, 2H), 5.65 (q, J = 8.0 Hz, 1H), 5.08-5.04 (m, 1H), 4.49-4.48 (m, 2H), 4.09 (q, J = 8.0 Hz, 1H), 3.11-3.08 (m, 1H), 3.69 (s, 3H), 3.23-3.16 (m, 3H), 2.80-2.57 (m, 5H), 2.54 (s, 3H), 2.14-2.19 (m, 8H), 1.58(d, J = 4.0 Hz, 3H). |
| 1051 | 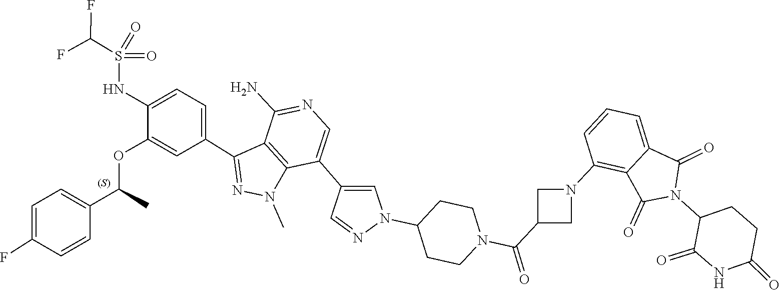 | N-(4-{4-amino-7-[1-(1-{1-[2-(2,6-dioxopiperidin-3-yl)- 1,3-dioxo-2,3-dihydro-1H-isoindol-4-yl]azetidine-3-carbonyl} piperidin-4-yl)-1H-pyrazol-4-yl]-1-methyl- 1H-pyrazolo[4,3-c]pyridin-3-yl}-2-[(1S)-1-(4-fluorophenyl)ethoxy]phenyl)-1,1-difluoromethane- sulfonamide From Intermediate L10. LCMS (Method A2): 2.90 min, m/z: 980.3 [M + H]⁺. ¹H NMR (400 MHZ, DMSO-d₆): 11.07 (s, 1H), 8.22 (s, 2H), 8.00 (s, 1H), 7.67-7.46 (m, 6H), 7.33 (d, J = 8.1 Hz, 1H), 7.12 (t, J = 9.0 Hz, 2H), 6.91 (d, J = 8.3 Hz, 1H), 6.86 (d, J = 9.3 Hz, 1H), 6.33 (t, J = 54.6 Hz, 1H), 5.64 (d, J = 6.2 Hz, 2H), 5.12-5.01 (m, 1H), 4.55-4.39 (m, 3H), 4.29 (dt, J = 16.9, 7.2 Hz, 2H), 3.91 (q, J = 7.7, 7.2 Hz, 2H), 3.67 (s, 3H), 2.97-2.58 (m, 5H), 1.99 (ddt, J = 62.0, 37.0, 13.1 Hz, 5H), 1.50 (d, J = 6.4 Hz, 3H). |
| 1077 | 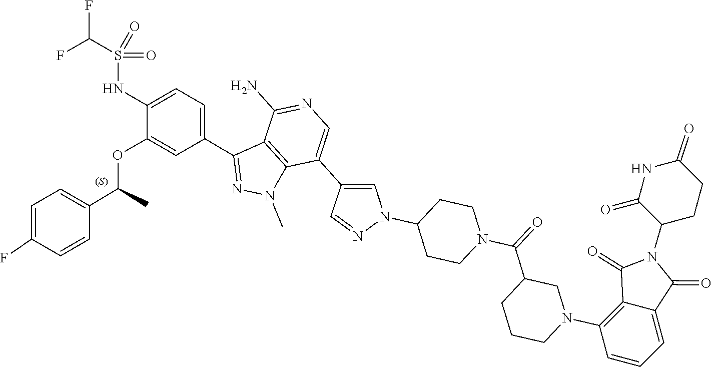 | N-(4-{4-amino-7-[1-(1-{1-[2-(2,6-dioxopiperidin-3-yl)- 1,3-dioxo-2,3-dihydro-1H-isoindol-4-yl]piperidine-3-carbonyl}piperidin-4-yl)-1H-pyrazol-4-yl]-1- methyl-1H-pyrazolo[4,3-c]pyridin-3-yl}-2-[(1S)-1- (4-fluorophenyl)ethoxy]phenyl)-1,1-difluoromethane- sulfonamide From Intermediate L11. LCMS (Method A): 2.89 min; m/z: 1008.2 [M + H]⁺. ¹H NMR (400 MHz, DMSO-d₆): 11.14-11.01 (m, 1H), 8.05-7.96 (m, 1H), 7.70 (dd, J = 8.4, 7.1 Hz, 1H), 7.60 (s, 1H), 7.50 (td, J = 5.7, 2.3 Hz, 3H), 7.40-7.30 (m, 3H), 7.12 (t, J = 8.9 Hz, 2H), 6.90 (s, 1H), 6.82 (d, J = 2.3 Hz, 1H), 6.19 (d, J = 55.0 Hz, 2H), 5.65 (d, J = 6.6 Hz, 1H), 5.63 (s, 2H), 5.17-5.06 (m, 1H), 4.50 (s, 2H), 4.36-4.16 (m, 1H), 3.77 (d, J = 30.1 Hz, 1H), 3.66 (s, 3H), 3.08 (s, 1H), 2.87 (s, 3H), 2.67 (p, J = 1.8 Hz, 1H), 2.33 (p, J = 1.9 Hz, 1H), 2.11 (d, J = 12.9 Hz, 3H), 1.99 (s, 3H), 1.79 (s, 3H), 1.49 (d, J = 6.4 Hz, 3H). |
| 1073 | 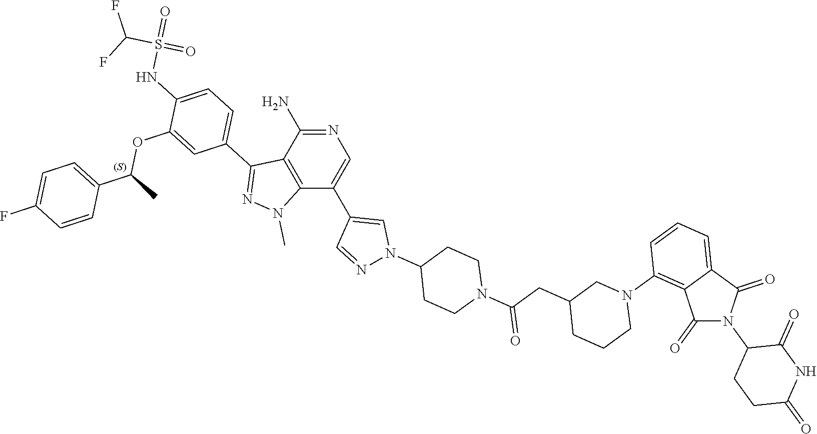 | N-[4-(4-amino-7-{1-[1-(2-{1-[1,3-dioxo-2-(2-oxo- piperidin-3-2,3-yl)-dihydro-1H-isoindol-4-yl]piperidin-3-yl}acetyl)piperidin-4-yl]-1H-pyrazol-4-yl}-1-methyl-1H- pyrazolo[4,3-c]pyridin-3-yl)-2-[(1S)-1-(4-fluorophenyl) ethoxy]phenyl]-1,1-difluoromethanesulfonamide From Intermediate L12. LCMS (Method A): 2.89 min, m/z 1022.1 [M + H]⁺. ¹H NMR (400 MHZ, DMSO-d₆): 11.10 (s, 1H), 8.09 (d, J = 7.7 Hz, 1H), 7.96 (s, 1H), 7.69 (dd, J = 8.0, 5.8 Hz, 2H), 7.57 (s, 3H), 7.48-7.36 (m, 2H), 7.31 (dd, J= 7.3, 4.0 Hz, 1H), 7.25-7.10 (m, 4H), 7.04 (s, 2H), 5.65 (d, J = 6.4 Hz, 1H), 5.17-5.04 (m, 1H), 4.52 (s, 2H), 4.06 (s, 1H), 3.74 (s, 3H), 3.67 (s, 2H), 2.94 (s, 1H), 2.84 (s, 1H), 2.69 (d, J = 5.1 Hz, 1H), 2.61 (s, 1H), 2.40-2.33 (m, 2H), 2.11 (s, 4H), 2.01 (d, J = 7.1 Hz, 2H), 1.80 (d, J = 44.0 Hz, 4H), 1.60 (d, J = 6.2 Hz, 3H). |
| 1072 | 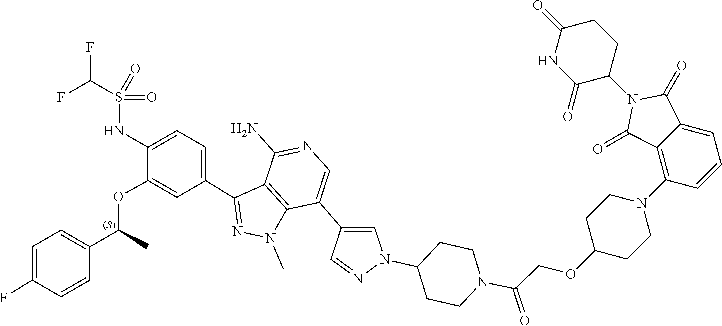 | N-{4-[4-amino-7-(1-{1-[2-({1-[2-(2,6-dioxopiperidin- 3-yl)-1,3-dioxo-2,3-dihydro-1H-isoindol-4-yl]piperidin- 4-yl}oxy)acetyl]piperidin-4-yl}-1H-pyrazol-4-yl)-1- methyl-1H-pyrazolo[4,3-c]pyridin-3-yl]-2-[(1S)-1-(4- fluorophenyl)ethoxy]phenyl}-1,1-difluoromethane- sulfonamide From Intermediate L13. LCMS (Method A): 2. 08 min, m/z: 1038.2 [M + H]⁺. ¹H NMR (400 MHZ, DMSO-d₆): 12.68 (s, 1H), 11.08 (s, 1H), 10.60 (s, 1H), 8.12 (d, J = 9.1 Hz, 1H), 7.73-7.64 (m, 2H), 7.63-7.54 (m, 3H), 7.44 (d, J = 8.1 Hz, 1H), 7.34 (t, J = 8.4 Hz, 2H), 7.22-7.10 (m, 5H), 6.97 (d, J = 52.6 Hz, 2H), 5.64 (t, J = 6.3 Hz, 1H), 5.12-5.06 (m, 1H), 4.52 (d, J = 11.6 Hz, 1H), 4.45 (d, J = 15.2 Hz, 2H), 4.26 (d, J = 6.0 Hz, 2H), 4.02 (s, 1H), 3.73 (s, 3H), 3.62 (s, 1H), 3.54 (d, J = 12.1 Hz, 2H), 3.09 (s, 3H), 2.85 (d, J = 16.5 Hz, 3H), 2.61 (s, 1H), 2.10 (s, 2H), 2.03 (s, 3H), 1.70 (s, 3H), 1.59 (d, J = 6.3 Hz, 3H). |
| 1070 | 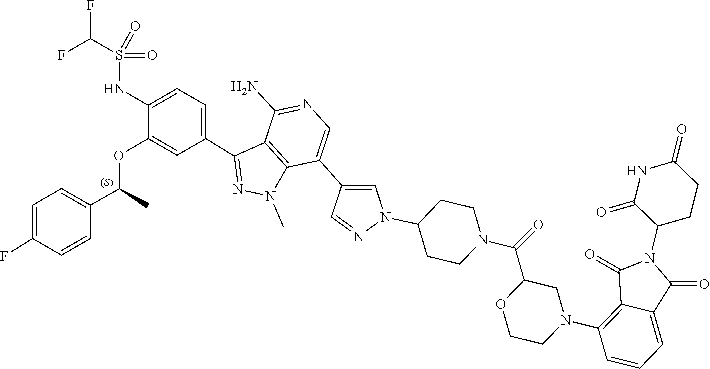 | N-(4-{4-amino-7-[1-(1-{4-[2-(2,6-dioxopiperidin- 3-yl)-1,3-dioxo-2,3-dihydro-1H-isoindol-4- yl]morpholine-2-carbonyl}piperidin-4-yl)-1H- pyrazol-4-yl]-1-methyl-1H-pyrazolo[4,3-c]pyridin- 3-yl}-2-[(1S)-1-(4-fluorophenyl)ethoxy]phenyl)- 1,1-difluoromethanesulfonamide From Intermediate L14. LCMS (Method C): 3.51 min, m/z: 505.2 [M + 2H]²⁺. ¹H NMR (400 MHZ, DMSO-d₆): 11.09 (d, J = 8.2 Hz, 1H), 8.03 (d, J = 5.4 Hz, 1H), 7.75 (t, J = 7.8 Hz, 1H), 7.63 (s, 1H), 7.61-7.54 (m, 3H), 7.41 (dd, J = 8.0, 4.2 Hz, 3H), 7.22-7.09 (m, 5H), 6.96 (d, J = 52.7 Hz, 1H), 5.84 (s, 2H), 5.66 (d, J = 6.5 Hz, 1H), 5.11 (dd, J = 12.7, 5.5 Hz, 1H), 4.49 (d, J = 32.5 Hz, 4H), 4.21 (t, J = 16.3 Hz, 2H), 4.02 (d, J = 11.3 Hz, 1H), 3.91-3.75 (m, 3H), 3.70 (s, 3H), 3.17 (d, J = 4.8 Hz, 2H), 3.08 (s, 3H), 2.86 (s, 3H), 2.14 (d, J = 13.6 Hz, 3H), 2.06-1.95 (m, 3H), 1.85 (d, J = 12.0 Hz, 1H), 1.59 (d, J = 6.3 Hz, 3H). |
| 1081 | 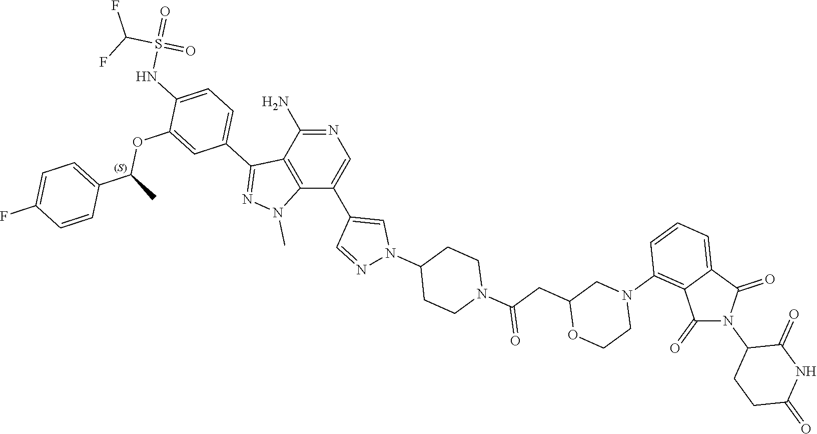 | N-[4-(4-amino-7-{1-[1-(2-{4-[2-(2,6-dioxopiperidin- 3-yl)-1,3-dioxo-2,3-dihydro-1H-isoindol- 4-yl]morpholin-2-yl}acetyl)piperidin-4-yl]-1H- pyrazol-4-yl}-1-methyl-1H-pyrazolo[4,3-c]pyridin- 3-yl)-2-[(1S)-1-(4-fluorophenyl)ethoxy]phenyl]- 1,1-difluoromethanesulfonamide From Intermediate L15. LCMS (Method C): 3.68 min, m/z: 1025.1 [M + H]⁺. ¹H NMR (400 MHZ, DMSO-d₆): 11.10 (s, 1H), 8.10 (d, J = 28.7 Hz, 3H), 7.72 (d, J = 7.6 Hz, 1H), 7.66 (s, 1H), 7.60-7.52 (m, 3H), 7.45-7.35 (m, 3H), 7.21-7.11 (m, 5H), 7.04 (s, 1H), 6.91 (s, 1H), 6.65 (s, 1H), 5.65 (d, J = 6.4 Hz, 1H), 5.32 (t, J = 4.8 Hz, 1H), 5.11 (d, J = 12.8 Hz, 2H), 4.51 (s, 3H), 4.04 (s, 3H), 3.92 (d, J = 11.1 Hz, 2H), 3.72 (s, 5H), 3.64-3.55 (m, 2H), 3.00 (d, J = 2.6 Hz, 2H), 2.93-2.72 (m, 6H), 2.67 (d, J = 2.0 Hz, 2H), 2.54 (s, 2H), 2.33 (s, 1H), 2.11 (s, 3H), 2.00 (q, J = 6.9, 6.3 Hz, 5H), 1.84 (s,2H), 1.59 (d, J = 6.2 Hz, 3H), 1.46 (s, 2H), 1.24 (s, 12H), 0.85 (t, J = 6.6 Hz, 2H). |
| 1074 | 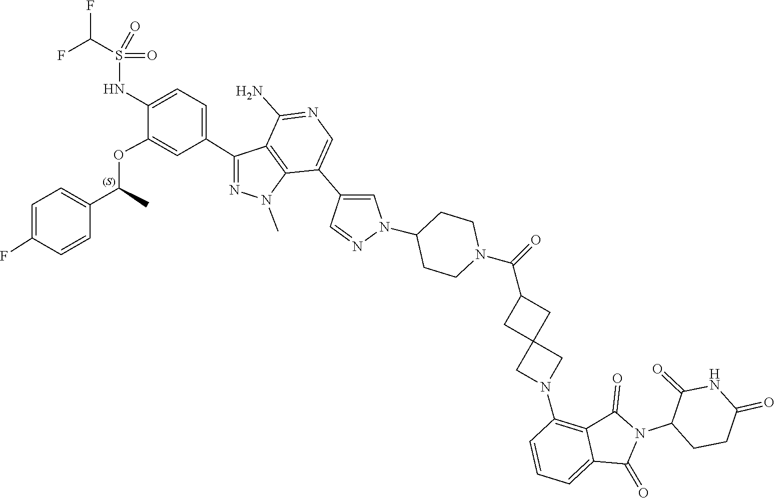 | N-(4-{4-amino-7-[1-(1-{2-[2-(2,6-dioxopiperidin- 3-yl)-1,3-dioxo-2,3-dihydro-1H-isoindol- 4-yl]-2-azaspiro[3.3]heptane-6-carbonyl}piperidin- 4-yl)-1H-pyrazol-4-yl]-1-methyl-1H-pyrazolo[4,3- c]pyridin-3-yl}-2-[(1S)-1-(4-fluorophenyl)ethoxy] phenyl)-1,1-difluoromethanesulfonamide From Intermediate L16. LCMS (Method A): 2.62 min, m/z: 1020.1 [M + H]⁺. ¹H NMR (400 MHZ, DMSO-d₆): 11.07 (s, 1H), 8.14 (s, 1H), 8.05 (s, 1H), 7.64 (s, 1H), 7.57 (d, J = 6.7 Hz, 3H), 7.42 (d, J = 8.0 Hz, 1H), 7.22-7.10 m, 4H), 6.77 (d, J = 8.5 Hz, 1H), 6.15 (s, 1H), 5.65 (d, J = 6.4 Hz, 1H), 5.05 (dd, J = 12.7, 5.5 Hz, 1H), 4.59-4.44 (m, 2H), 4.27 (s, 2H), 4.09 (s, 2H), 3.86 (d, J = 13.6 Hz, 2H), 3.71 (s, 4H), 3.21-3.12 (m, 3H), 2.75 (d, J = 7.9 Hz, 3H), 2.62-2.55 (m, 1H), 2.42 (dq, J = 11.7, 5.7, 4.0 Hz, 3H), 1.88-1.69 (m, 2H), 1.59 (d, J = 6.3 Hz, 3H). |
| 1093 | 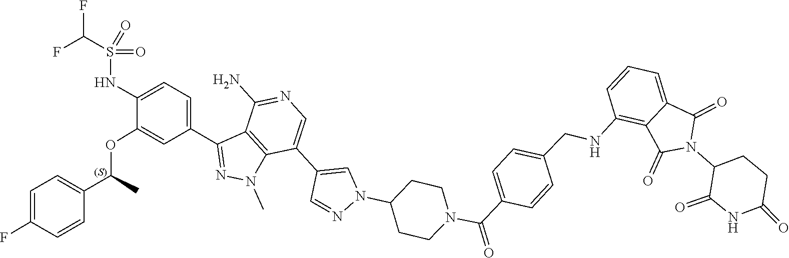 | N-{4-[4-amino-7-(1-{1-[4-({[2-(2,6-dioxopiperidin- 3-yl)-1,3-dioxo-2,3-dihydro-1H-isoindol-4- yl]amino}methyl)benzoyl]piperidin- 4-yl}-1H-pyrazol-4-yl)-1-methyl-1H-pyrazolo[4,3- c]pyridin-3-yl]-2-[(1S)-1-(4-fluorophenyl)ethoxy] phenyl}-1,1-difluoromethanesulfonamide From Intermediate L17. LCMS (Method A): 2.88 min, m/z: 1030.2 [M + H]⁺. ¹H NMR (400 MHZ, DMSO-d₆): 11.11 (s, 1H), 8.02 (s, 1H), 7.61 (s, 1H), 7.58-7.49 (m, 4H), 7.40 (dd, J = 20.8, 6.4 Hz, 5H), 7.31 (t, J = 6.4 Hz, 1H), 7.15 (t, J = 8.7 Hz, 2H), 7.07-6.94 (m, 4H), 6.76 (t, J = 53.5 Hz, 1H), 5.65 (d, J = 6.3 Hz, 3H), 5.07 (dd, J = 12.9, 5.4 Hz, 1H), 4.62 (d, J = 6.3 Hz, 2H), 4.53 (td, J = 11.0, 5.4 Hz, 2H), 3.67 (s, 3H), 3.02 (s, 1H), 2.96-2.82 (m, 1H), 2.65-2.52 (m, 3H), 2.23-1.85 (m, 6H), 1.55 (d, J = 6.3 Hz, 3H). |
| 1112 | 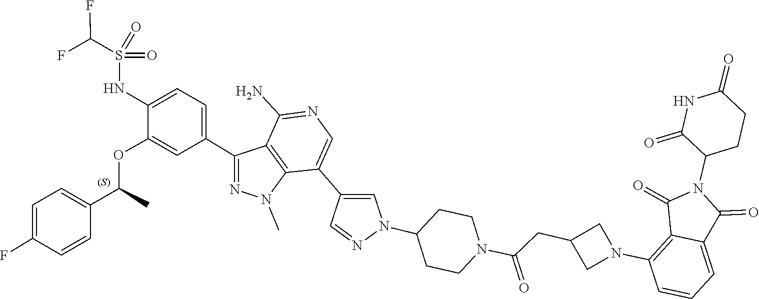 | N-[4-(4-amino-7-{1-[1-(2-{1-[2-(2,6-dioxopiperidin- 3-yl)-1,3-dioxo-2,3-dihydro-1H-isoindol-4-yl]azetidin- 3-yl}acetyl)piperidin-4-yl]-1H-pyrazol-4-yl}-1-methyl- 1H-pyrazolo[4,3-c]pyridin-3-yl)-2-[(1S)-1-(4- fluorophenyl)ethoxy]phenyl]- 1,1-difluoromethanesulfonamide From Intermediate L18. LCMS (method A): 2.95 min, m/z: 994.3 [M + H]⁺. ¹H NMR (400 MHZ, DMSO-d₆): 10.99 (s, 1H), 8.08 (s, 1H), 7.92 (s, 2H), 7.53 (s, 1H), 7.44 (d, J = 3.7 Hz, 3H), 7.28 (s, 1H), 7.14 (s, 2H), 7.11-7.00 (m, 3H), 6.90 (s, 1H), 6.85 (s, 1H), 6.72 (s, 1H), 6.59 (s, 2H), 5.57 (s, 3H), 5.26 (s, 2H), 4.98 (s, 1H), 4.41 (s, 3H), 4.29 (s, 3H), 3.90 (s, 2H), 3.78 (s, 3H), 3.60 (s, 3H), 2.76 (d, J = 7.2 Hz, 3H), 2.60 (s, 2H), 2.26 (s, 1H), 2.03 (s, 3H), 1.46 (d, J = 6.3 Hz, 3H), 1.38 (d, J = 6.9 Hz, 4H). |
| 1069 | 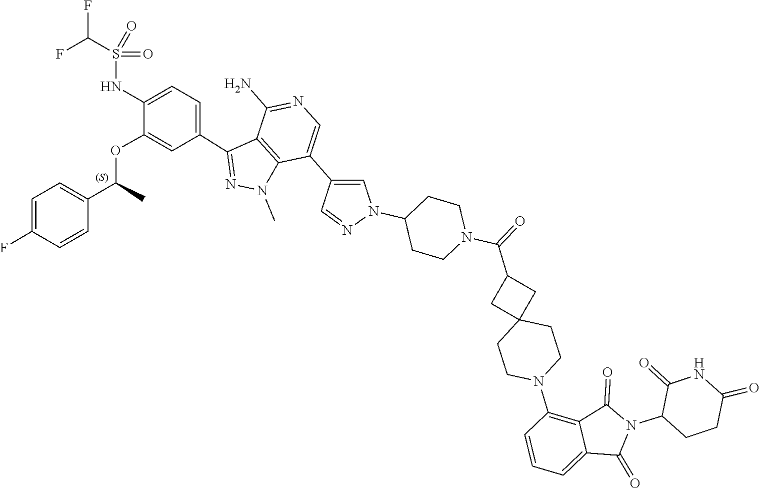 | N-(4-{4-amino-7-[1-(1-{7-[2-(2,6-dioxopiperidin- 3-yl)-1,3-dioxo-2,3-dihydro-1H-isoindol-4-yl]- 7-azaspiro[3.5]nonane-2-carbonyl}piperidin- 4-yl)-1H-pyrazol-4-yl]-1-methyl-1H-pyrazolo[4,3- c]pyridin-3-yl}-2-[(1S)-1-(4-fluorophenyl)ethoxy] phenyl)-1,1-difluoromethanesulfonamide From Intermediate L19. LCMS (Method A2): 3.01 min, m/z: 524.8 [M + 2H]²⁺. ¹H NMR (400 MHZ, DMSO-d₆): 11.08 (s, 1H), 8.03 (s, 1H), 7.70-7.64 (m, 1H), 7.63 (s, 1H), 7.58 (d, J = 5.5 Hz, 1H), 7.56 (d, J = 5.1 Hz, 2H), 7.41 (d, J = 8.0 Hz, 1H), 7.31 (dt, J = 15.4, 8.1 Hz, 2H), 7.17 (t, J = 7.5 Hz, 2H), 7.15-7.11 (m, 2H), 6.96 (d, J = 52.6 Hz, 1H), 5.99 (s, 2H), 5.65 (d, J = 6.4 Hz, 1H), 5.09 (dd, J = 12.9, 5.4 Hz, 1H), 4.57-4.44 (m, 2H), 3.86 (d, J = 13.1 Hz, 1H), 3.70 (s, 3H), 3.40 (t, J = 8.8 Hz, 1H), 3.24 (d, J = 5.5 Hz, 2H), 3.15 (d, J = 8.4 Hz, 2H), 2.93-2.73 (m, 2H), 2.68-2.53 (m, 2H), 2.43-2.16 (m, 2H), 2.15-1.96 (m, 7H), 1.92-1.74 (m, 4H), 1.64 (d, J = 5.5 Hz, 2H), 1.59 (d, J = 6.2 Hz, 3H). |
| 1087 | 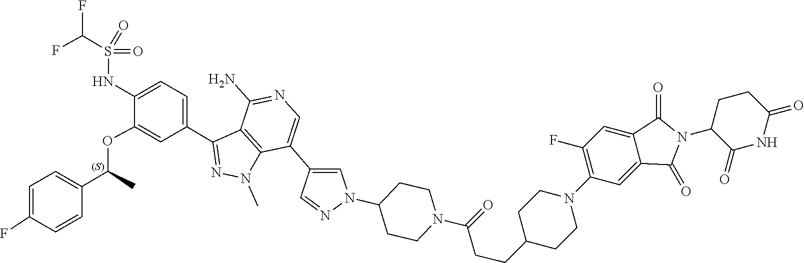 | N-[4-(4-amino-7-{1-[1-(3-{1-[2-(2,6-dioxopiperidin- 3-yl)-6-fluoro-1,3-dioxo-2,3-dihydro-1H-isoindol- 5-yl]piperidin-4-yl}propanoyl)piperidin-4-yl]-1H- pyrazol-4-yl}-1-methyl-1H-pyrazolo[4,3-c]pyridin- 3-yl)-2-[(1S)-1-(4-fluorophenyl)ethoxy]phenyl]- 1,1-difluoromethanesulfonamide From Intermediate L20. LCMS (Method A2): 2.95 min, m/z: 1054.4 [M + H]⁺. ¹H NMR (400 MHZ, DMSO-d₆): 11.10 (s, 1H), 8.03 (s, 1H), 7.69 (d, J = 11.4 Hz, 1H), 7.62 (s, 1H), 7.59-7.53 (m, 3H), 7.42 (dd, J = 15.1, 7.7 Hz, 2H), 7.21-7.07 (m, 5H), 6.99 (s, 1H), 5.95 (s, 2H), 5.65 (q, J = 6.3 Hz, 1H), 5.09 (dd, J = 12.8, 5.4 Hz, 1H), 4.49 (dt, J = 11.2, 6.1 Hz, 2H), 4.02 (d, J = 13.3 Hz, 1H), 3.69 (s, 3H), 3.60 (d, J = 12.1 Hz, 2H), 3.27-3.14 (m, 2H), 2.90-2.84 (m, 2H), 2.83- 2.69 (m, 2H), 2.67-2.55 (m, 2H), 2.42 (t, J = 7.4 Hz, 2H), 2.10 (s, 2H), 1.82 (d, J = 12.4 Hz, 3H), 1.57 (d, J = 6.3 Hz, 3H), 1.50 (q, J = 9.7, 8.3 Hz, 3H), 1.38 (d, J = 3.1 Hz, 1H), 1.34 (s, 2H). |
| 1098 | 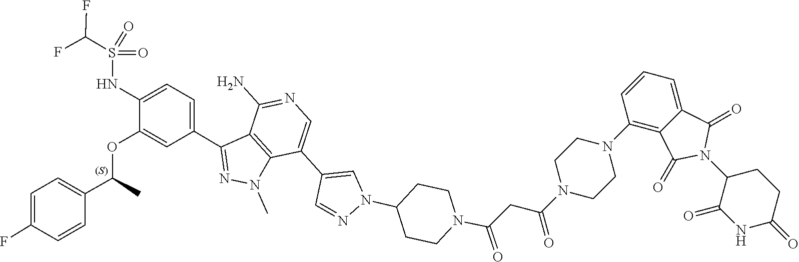 | N-[4-(4-amino-7-{1-[1-(3-{4-[2-(2,6-dioxopiperidin- 3-yl)-1,3-dioxo-2,3-dihydro-1H-isoindol-4- yl]piperazin-1-yl}-3-oxopropanoyl)piperidin- 4-yl]-1H-pyrazol-4-yl}-1-methyl-1H-pyrazolo[4,3- c]pyridin-3-yl)-2-[(1S)-1-(4-fluorophenyl)ethoxy] phenyl]-1,1-difluoromethanesulfonamide From Intermediate L21. LCMS (Method A2): 2.73 min, m/z: 526.3 [M + 2H]²⁺. ¹H NMR (400 MHZ, DMSO-d₆): 11.09 (d, J = 2.1 Hz, 1H), 10.57 (s, 1H), 8.00 (s, 1H), 7.72 (dd, J = 8.4, 7.2 Hz, 1H), 7.61 (s, 1H), 7.59-7.51 (m, 3H), 7.40 (d, J = 3.0 Hz, 1H), 7.39-7.32 (m, 2H), 7.17 (d, J = 8.9 Hz, 2H), 7.16-7.12 (m, 1H), 7.12-7.04 (m, 2H), 6.89 (d, J = 52.9 Hz, 1H), 5.82-5.61 (m, 3H), 5.11 (dd, J = 12.9, 5.4 Hz, 1H), 4.56-4.43 (m, 2H), 4.03 (q, J = 7.1 Hz, 1H), 3.93 (d, J = 13.4 Hz, 1H), 3.69 (s, 3H), 3.67-3.59 (m, 4H), 3.28-3.15 (m, 3H), 2.94- 2.77 (m, 2H), 2.65-2.54 (m, 2H), 2.11 (d, J = 12.0 Hz, 2H), 2.00 (d, J = 8.8 Hz, 3H), 1.90-1.78 (m, 1H), 1.58 (d, J = 6.2 Hz, 3H). |
| 1141 | 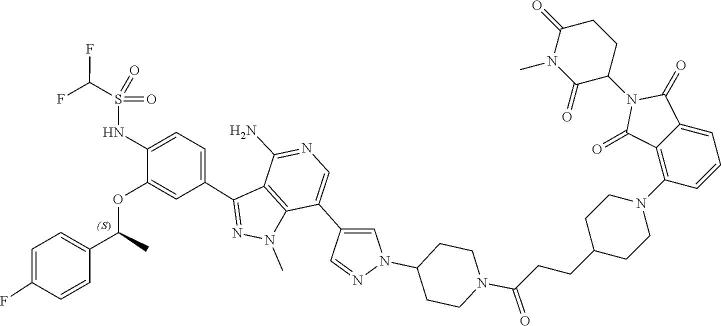 | N-[4-(4-amino-1-methyl-7-{1-[1-(3-{1-[2-(1- methyl-2,6-dioxopiperidin-3-yl)-1,3-dioxo-2,3- dihydro-1H-isoindol-4-yl]piperidin-4-yl}propanoyl) piperidin-4-yl]-1H-pyrazol-4-yl}-1H-pyrazolo[4,3- c]pyridin-3-yl)-2-[(1S)-1-(4-fluorophenyl)ethoxy] phenyl]-1,1-difluoromethanesulfonamide From Intermediate L22. LCMS (Method A2): 3.02 min, m/z: 1050.3 [M + H]⁺. ¹H NMR (400 MHz, DMSO-d₆): 8.01 (s, 1H), 7.70- 7.64 (m, 1H), 7.61 (s, 1H), 7.56 (d, J = 5.7 Hz, 1H), 7.54 (t, J = 2.8 Hz, 2H), 7.39 (d, J = 8.0 Hz, 1H), 7.32 (dd, J = 9.8, 7.8 Hz, 2H), 7.15 (t, J = 8.8 Hz, 2H), 7.11-7.06 (m, 2H), 6.89 (t, J = 53.2 Hz, 1H), 5.76 (d, J = 4.1 Hz, 2H), 5.65 (d, J = 6.4 Hz, 1H), 5.16 (dd, J = 13.0, 5.4 Hz, 1H), 4.50 (d, J = 11.8 Hz, 2H), 4.02 (d, J = 13.4 Hz, 1H), 3.71 (s, 1H), 3.68 (s, 3H), 3.01 (s, 3H), 2.95-2.71 (m, 6H), 2.58-2.52 (m, 2H), 2.42 (t, J = 6.7 Hz, 2H), 2.08 (d, J = 17.8 Hz, 3H), 1.91 (d, J = 11.4 Hz, 1H), 1.82 (s, 1H), 1.79 (s, 1H), 1.57 (d, J = 6.3 Hz, 4H), 1.55-1.49 (m, 2H), 1.35 (d, J = 11.6 Hz, 1H), 0.94 (d, J = 5.8 Hz, 1H), 0.84 (td, J = 9.3, 8.1, 5.7 Hz, 1H). |
| 1118 | 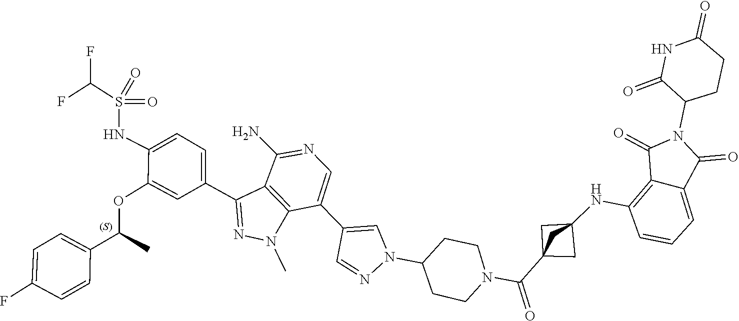 | N-[4-(4-amino-7-{1-[1-(3-{[2-(2,6-dioxopiperidin- 3-yl)-1,3-dioxo-2,3-dihydro-1H-isoindol-4- yl]amino}bicyclo[1.1.1]pentane-1-carbonyl)piperidin- 4-yl]-1H-pyrazol-4-yl}-1-methyl-1H-pyrazolo[4,3- c]pyridin-3-yl)-2-[(1S)-1-(4-fluorophenyl)ethoxy] phenyl]-1,1-difluoromethanesulfonamide From Intermediate L23. LCMS (Method A): 2.95 min, m/z: 1006.4 [M + H]⁺. ¹H NMR (400 MHZ, DMSO-d₆): 11.09 (s, 1H), 8.59 (s, 3H), 8.14 (s, 1H), 7.73 (s, 1H), 7.66 (s, 1H), 7.59 (dd, J = 8.4, 5.5 Hz, 2H), 7.46 (dd, J = 16.8, 8.4 Hz, 3H), 7.18 (t, J = 8.5 Hz, 3H), 7.14 (s, 1H), 7.04 (s, 1H), 7.02 (d, J = 2.5 Hz, 1H), 6.99 (s, 1H), 6.53 (s, 2H), 5.66 (q, J = 6.2 Hz, 1H), 5.38 (s, 1H), 5.27 (s, 1H), 5.05 (dd, J = 12.9, 5.4 Hz, 2H), 4.57 (dq, J = 11.4, 6.5, 5.5 Hz, 1H), 3.75 (s, 3H), 2.88 (ddd, J = 17.4, 14.1, 5.4 Hz, 2H), 2.62 (d, J = 3.5 Hz, 3H), 2.12 (s, 4H), 2.06-1.96 (m, 3H), 1.60 (d, J = 6.2 Hz, 3H) |
| 1110 | 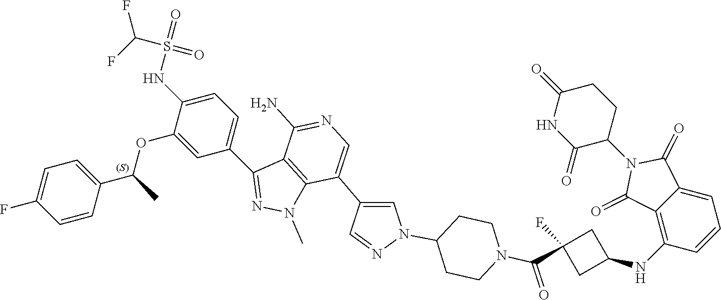 | N-{4-[4-amino-1-methyl-7-(1-{1-[(trans)-3- {[2-(2,6-dioxopiperidin-3-yl)-1,3-dioxo-2,3- dihydro-1H-isoindol-4-yl]amino}-1-fluoro- cyclobutane carbonyl]piperidin-4-yl}-1H-pyrazol- 4-yl)-1H-pyrazolo[4,3-c]pyridin-3-yl]-2-[(1S)-1- (4-fluorophenyl)ethoxy]phenyl}-1,1- difluoromethanesulfonamide From Intermediate L24. Diastereomers separated by prep-HPLC. LCMS (Method A): 2.01 min; m/z: 1012.9 [M + H]⁺. ¹H NMR (400 MHZ, DMSO-d₆): 11.10 (d, J = 3.5 Hz, 1H), 8.14 (s, 1H), 8.02 (d, J = 8.6 Hz, 1H), 7.61 (dd, J = 7.8, 5.9 Hz, 2H), 7.58-7.51 (m, 3H), 7.39 (d, J = 8.0 Hz, 1H), 7.20-7.01 (m, 6H), 6.68 (dd, J = 37.0, 6.8 Hz, 2H), 5.65 (dd, J = 12.8, 6.4 Hz, 3H), 5.07 (dt, J = 12.9, 4.9 Hz, 1H), 4.54 (d, J = 10.4 Hz, 2H), 4.48-4.37 (m, 2H), 3.93 (d, J = 13.5 Hz, 1H), 3.68 (d, J = 5.5 Hz, 3H), 2.92 (dd, J = 19.8, 9.5 Hz, 3H), 2.59 (d, J = 14.7 Hz, 2H), 2.15 (d, J = 12.0 Hz, 2H), 2.08-2.01 (m, 1H), 1.92 (d, J = 9.0 Hz, 2H), 1.56 (d, J = 6.2 Hz, 3H). |
| 1111 | 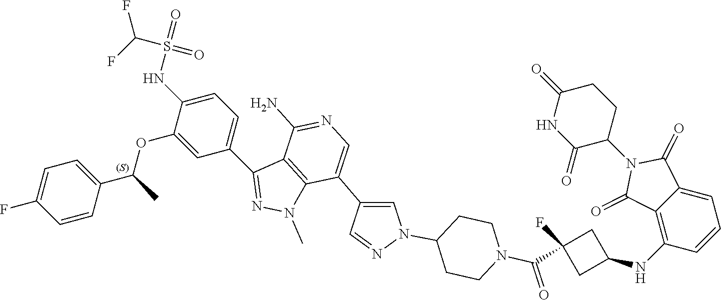 | N-{4-[4-amino-1-methyl-7-(1-{1-[(cis)-3-{[2-(2,6- dioxopiperidin-3-yl)-1,3-dioxo-2,3-dihydro-1H- isoindol-4-yl]amino}-1-fluorocyclobutane- carbonyl]piperidin-4-yl}-1H-pyrazol-4-yl)- 1H-pyrazolo[4,3-c]pyridin-3-yl]-2-[(1S)-1-(4- fluorophenyl)ethoxy]phenyl}-1,1-difluoro- methanesulfonamide From Intermediate L24. Diastereomers separated by prep-HPLC. LCMS (Method A): 2.01 min; m/z: 1012.9 [M + H]⁺. ¹H NMR (400 MHZ, DMSO-d₆): 11.10 (d, J = 3.5 Hz, 1H), 8.14 (s, 1H), 8.02 (d, J = 8.6 Hz, 1H), 7.61 (dd, J = 7.8, 5.9 Hz, 2H), 7.58-7.51 (m, 3H), 7.39 (d, J = 8.0 Hz, 1H), 7.20-7.01 (m, 6H), 6.68 (dd, J = 37.0, 6.8 Hz, 2H), 5.65 (dd, J = 12.8, 6.4 Hz, 3H), 5.07 (dt, J = 12.9, 4.9 Hz, 1H), 4.54 (d, J = 10.4 Hz, 2H), 4.48-4.37 (m, 2H), 3.93 (d, J = 13.5 Hz, 1H), 3.68 (d, J = 5.5 Hz, 3H), 2.92 (dd, J = 19.8, 9.5 Hz, 3H), 2.59 (d, J = 14.7 Hz, 2H), 2.15 (d, J = 12.0 Hz, 2H), 2.08-2.01 (m, 1H), 1.92 (d, J = 9.0 Hz, 2H), 1.56 (d, J = 6.2 Hz, 3H). |
| 1129 | 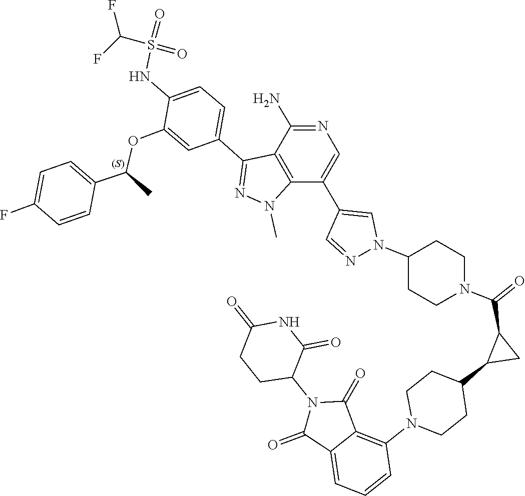 | N-[4-(4-amino-7-{1-[1-(2-{1-[2-(2,6-dioxopiperidin- 3-yl)-1,3-dioxo-2,3-dihydro-1H-isoindol-4- yl]piperidin-4-yl}-(cis)-cyclopropanecarbonyl) piperidin-4-yl]-1H-pyrazol-4-yl}-1-methyl-1H- pyrazolo[4,3-c]pyridin-3-yl)-2-[(1S)-1-(4- fluorophenyl)ethoxy]phenyl]-1,1-difluoromethane- sulfonamide From Intermediate L25. Diastereomers separated by prep-TLC (DCM:MeOH = 30:1). LCMS (Method A): 2.15 min, m/z: 1048.4 [M + H]⁺. ¹H NMR (400 MHZ, CDCl₃): 7.66 (d, J = 8.3 Hz, 1H), 7.60-7.50 (m, 3H), 7.37 (d, J = 7.1 Hz, 1H), 7.34-7.29 (m, 2H), 7.20 (d, J = 8.0 Hz, 1H), 7.17 (d, J = 8.4 Hz, 1H), 7.07-6.99 (m, 2H), 6.35 (t, J = 53.5 Hz, 1H), 5.84 (s, 1H), 5.46 (t, J = 6.8 Hz, 1H), 4.95 (dd, J = 11.9, 5.6 Hz, 1H), 4.81 (s, 1H), 4.51 (s, 1H), 4.39 (d, J = 13.4 Hz, 1H), 3.78 (s, 4H), 3.63 (s, 1H), 3.34 (s, 1H), 2.95-2.69 (m, 6H), 2.41 (s, 1H), 2.36-2.21 (m, 2H), 2.06 (s, 1H), 2.02-1.97 (m, 1H), 1.93 (d, J = 12.0 Hz, 1H), 1.78 (s, 2H), 1.69 (dd, J = 6.4, 1.9 Hz, 3H), 1.31 (s, 2H), 0.78-0.72 (m, 1H). |
| 1130 | 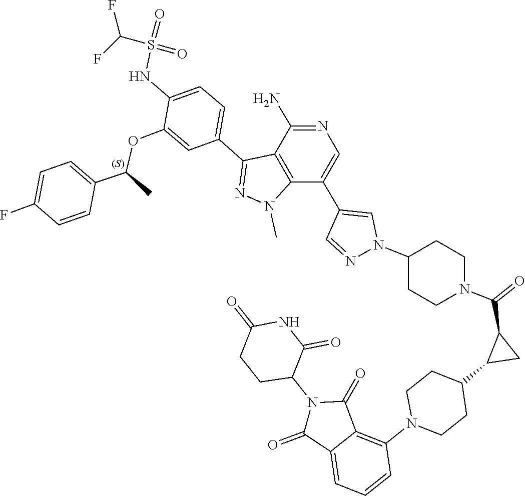 | N-[4-(4-amino-7-{1-[1-(2-{1-[2-(2,6-dioxopiperidin- 3-yl)-1,3-dioxo-2,3-dihydro-1H-isoindol-4- yl]piperidin-4-yl}-(trans)-cyclopropanecarbonyl) piperidin-4-yl]-1H-pyrazol-4-yl}-1-methyl-1H- pyrazolo[4,3-c]pyridin-3-yl)-2-[(1S)-1-(4- fluorophenyl)ethoxy]phenyl]-1,1-difluoromethane- sulfonamide From Intermediate L25. Diastereomers separated by prep-TLC (DCM:MeOH = 30:1). LCMS (Method A): 2.15 min, m/z: 1048.4 [M + H]⁺. ¹H NMR (400 MHZ, CDCl₃): 7.66 (d, J = 8.2 Hz, 1H), 7.61-7.51 (m, 3H), 7.39-7.28 (m, 2H), 7.20- 7.13 (m, 1H), 7.06-6.98 (m, 2H), 6.36 (t, J = 53.5 Hz, 1H), 6.01 (s, 1H), 5.46 (d, J = 6.4 Hz, 1H), 4.95 (dd, J = 11.9, 5.5 Hz, 1H), 4.75 (s, 1H), 4.53-4.35 (m, 1H), 3.77 (s, 3H), 3.34 (s, 1H), 3.09- 2.70 (m, 7H), 2.31 (s, 1H), 2.08 (t, J = 10.8 Hz, 1H), 2.02-1.78 (m, 3H), 1.68 (d, J = 6.2 Hz, 3H), 1.30 (s, 2H), 1.04 (d, J = 10.2 Hz, 1H), 0.86 (s, 1H), 0.76 (s, 1H). |
| 1119 | 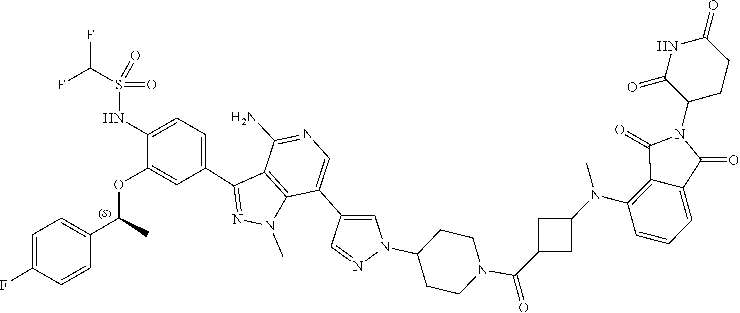 | N-{4-[4-amino-1-methyl-7-(1-{1-[(trans)-3-{[2-(2,6- dioxopiperidin-3-yl)-1,3-dioxo-2,3-dihydro-1H-isoindol- 4-yl](methyl)amino}cyclobutanecarbonyl]piperidin-4- yl}-1H-pyrazol-4-yl)-1H-pyrazolo[4,3-c]pyridin-3-yl]- 2-[(1S)-1-(4-fluorophenyl)ethoxy]phenyl}-1,1- difluoromethanesulfonamide From Intermediate L26. LCMS (Method B): 2.02 min, m/z: 504.8 [M + 2H]²⁺. ¹H NMR (400 MHZ, DMSO-d₆): 11.08 (d, J = 8.7 Hz, 1H), 8.12 (s, 1H), 7.71 (d, J = 3.2 Hz, 1H), 7.66-7.55 (m, 4H), 7.44 (d, J = 8.0 Hz, 1H), 7.29 (t, J = 6.1 Hz, 1H), 7.22 (s, 2H), 7.18 (t, J = 8.1 Hz, 3H), 7.11 (d, J = 11.2 Hz, 2H), 6.97 (d, J = 52.6 Hz, 1H), 5.64 (d, J = 6.4 Hz, 1H), 5.09 (dd, J = 13.4, 5.5 Hz, 1H), 4.52 (s, 1H), 4.20-4.06 (m, 1H), 3.91 (d, J = 12.6 Hz, 1H), 3.73 (s, 3H), 3.09 (s, 2H), 2.96 (d, J = 17.6 Hz, 3H), 2.85 (dt, J = 22.7, 8.4 Hz, 2H), 2.64 (d, J = 22.5 Hz, 4H), 2.43-2.23 (m, 2H), 2.09 (s, 2H), 2.07 (s, 2H), 1.83 (d, J = 13.2 Hz, 2H), 1.59 (d, J = 6.2 Hz, 3H). |
| 1094 | 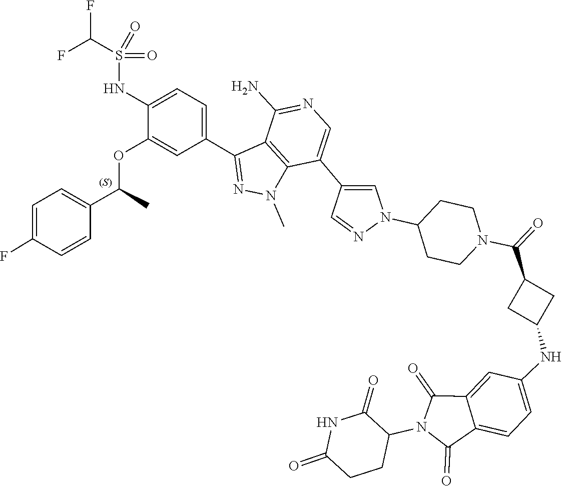 | N-{4-[4-amino-1-methyl-7-(1-{1-[(trans)-3-{[2- (2,6-dioxopiperidin-3-yl)-1,3-dioxo-2,3-dihydro- 1H-isoindol-5-yl]amino}cyclobutanecarbonyl] piperidin-4-yl}-1H-pyrazol-4-yl)-1H-pyrazolo[4,3- c]pyridin-3-yl]-2-[(1S)-1-(4-fluorophenyl)ethoxy] phenyl}-1,1-difluoromethanesulfonamide From Intermediate L27. LCMS (Method B): 3.68 min, m/z 995.2 [M + H]⁺. ¹H NMR (400 MHZ, DMSO-d₆): 11.05 (s, 1H), 8.00 (s, 1H), 7.58 (d, J = 8.2 Hz, 2H), 7.52-7.45 (m, 4H), 7.33 (d, J = 8.1 Hz, 1H), 7.12 (t, J = 8.9 Hz, 2H), 6.88 (dd, J = 8.2, 2.1 Hz, 1H), 6.81 (d, J = 2.1 Hz, 1H), 6.79-6.73 (m, 1H), 6.25 (t, J = 55.0 Hz, 1H), 5.68-5.61 (m, 2H), 5.02 (dd, J = 12.9, 5.4 Hz, 1H), 4.50 (dd, J = 14.9, 8.6 Hz, 2H), 3.97 (d, J = 6.9 Hz, 1H), 3.78 (d, J = 13.4 Hz, 1H), 3.66 (s, 3H), 3.49-3.42 (m, 1H), 3.21-3.14 (m, 1H), 2.84 (q, J = 14.6, 12.3 Hz, 2H), 2.73-2.58 (m, 3H), 2.33 (t, J = 1.9 Hz, 1H), 2.22-2.06 (m, 4H), 2.00 (q, J = 6.9 Hz, 2H), 1.86 (dd, J = 20.9, 1 0.4 Hz, 2H), 1.49 (d, J = 6.4 Hz, 3H). |
| 1106 | 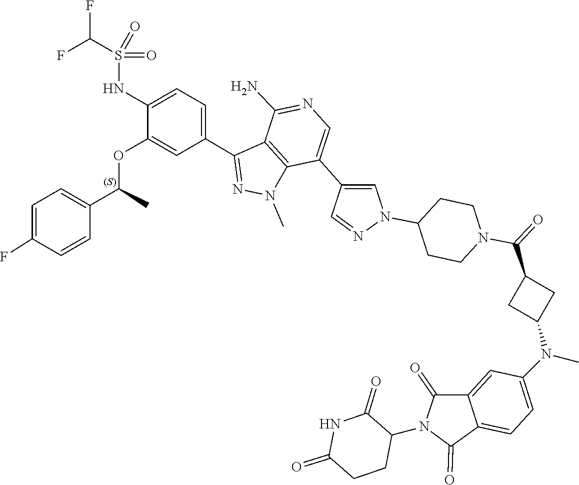 | N-{4-[4-amino-1-methyl-7-(1-{1-[(trans)-3-{[2-(2,6- dioxopiperidin-3-yl)-1,3-dioxo-2,3-dihydro-1H-isoindol- 5-yl](methyl)amino}cyclobutanecarbonyl]piperidin-4- yl}-1H-pyrazol-4-yl)-1H-pyrazolo[4,3-c]pyridin-3-yl]- 2-[(1S)-1-(4-fluorophenyl)ethoxy]phenyl}-1,1- difluoromethanesulfonamide From Intermediate L28. LCMS (Method B): 1.92 min, m/z 1009.4 [M + H]⁺. ¹H NMR (400 MHZ, DMSO-d₆): 11.08 (s, 1H), 8.12 (d, J = 1.8 Hz, 1H), 7.71 (d, J = 3.1 Hz, 1H), 7.68-7.55 (m, 4H), 7.44 (d, J = 8.0 Hz, 1H), 7.31- 7.09 (m, 8H), 6.97 (d, J = 52.6 Hz, 1H), 5.64 (q, J = 6.3 Hz, 1H), 5.09 (dd, J = 12.9, 5.4 Hz, 1H), 4.52 (t, J = 12.7 Hz, 1H), 4.14 (d, J = 7.9 Hz, 1H), 3.73 (d, J = 2.5 Hz, 3H), 3.24-3.06 (m, 2H), 2.96 (d, J = 17.6 Hz, 3H), 2.90-2.75 (m, 2H), 2.68-2.54 (m, 4H), 2.37-2.24 (m, 2H), 2.19-1.98 (m, 4H), 1.83 (d, J = 13.2 Hz, 2H), 1.59 (d, J = 6.2 Hz, 3H). |
| 1125 | 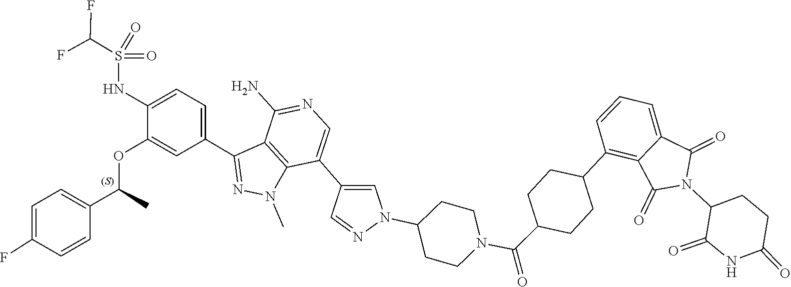 | N-(4-{4-amino-7-[1-(1-{4-[2-(2,6-dioxopiperidin- 3-yl)-1,3-dioxo-2,3-dihydro-1H-isoindol-4- yl]cyclohexane-carbonyl}piperidin-4-yl)-1H-pyrazol- 4-yl]-1-methyl-1H-pyrazolo[4,3-c]pyridin-3-yl}-2- [(1S)-1-(4-fluorophenyl)ethoxy] phenyl)-1,1-difluoromethanesulfonamide From Intermediate L29. LCMS (Method B): 2.03 min, m/z: 1008 [M + H]⁺. ¹H NMR (400 MHZ, DMSO-d₆): 11.11 (s, 1H), 8.04 (s, 1H), 7.95 (s, 1H), 7.82 (t, J = 7.6 Hz, 1H), 7.76-7.71 (m, 2H), 7.62 (s, 1H), 7.59-7.54 (m, 3H), 7.40 (d, J = 8.0 Hz, 1H), 7.19-7.07 (m, 4H), 5.65 (q, J = 6.3 Hz, 1H), 5.12 (dd, J = 12.8, 5.4 Hz, 1H), 4.03 (q, J = 7.1 Hz, 2H), 3.69 (s, 3H), 3.06 (d, J = 5.4 Hz, 1H), 2.89 (s, 5H), 2.73 (s, 4H), 2.17 (d, J = 7.5 Hz, 1H), 2.08-1.96 (m, 6H), 1.58 (d, J = 6.3 Hz, 3H), 1.17 (t, J = 7.1 Hz, 2H), 0.85 (t, J = 6.5 Hz, 2H). |
| 1126 | 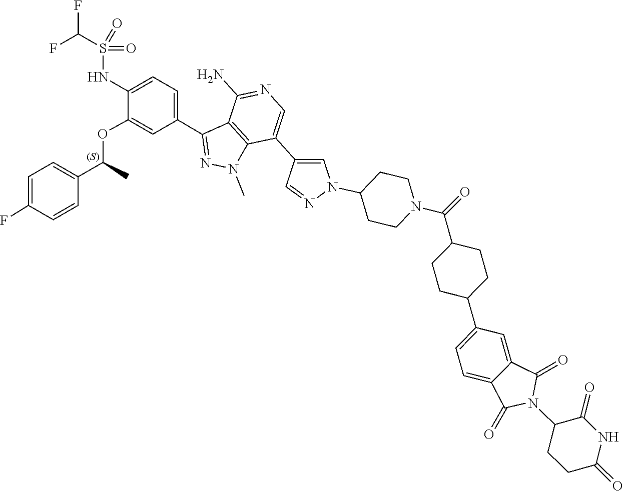 | N-(4-{4-amino-7-[1-(1-{4-[2-(2,6-dioxopiperidin- 3-yl)-1,3-dioxo-2,3-dihydro-1H-isoindol-5-yl]cyclohexanecarbonyl}piperidin-4-yl)-1H- pyrazol-4-yl]-1-methyl-1H-pyrazolo[4,3- c]pyridin-3-yl}-2-[(1S)-1-(4-fluorophenyl)ethoxy] phenyl)-1,1-difluoromethanesulfonamide From Intermediate L30. LCMS (Method B): 1.96 min, m/z: 504.3 [M + 2H]²⁺. ¹H NMR (400 MHZ, DMSO-d₆): 11.11 (s, 1H), 8.00 (s, 1H), 7.86 (d, J = 7.6 Hz, 1H), 7.75 (d, J = 9.0 Hz, 2H), 7.59 (s, 1H), 7.49 (q, J = 5.5 Hz, 3H), 7.33 (d, J = 8.2 Hz, 1H), 7.12 (t, J = 8.7 Hz, 2H), 6.92-6.85 (m, 1H), 6.81 (d, J = 2.1 Hz, 1H), 6.24 (t, J = 55.0 Hz, 1H), 5.66-5.61 (m, 2H), 5.12 (dd, J = 12.9, 5.4 Hz, 1H), 4.50 (d, J = 14.8 Hz, 2H), 4.05 (s, 1H), 3.66 (s, 3H), 3.17 (s, 1H), 3.03 (s, 1H), 2.93-2.83 (m, 2H), 2.75 (s, 1H), 2.59 (d, J = 16.4 Hz, 2H), 2.07 (t, J = 18.3 Hz, 5H), 1.83 (s, 4H), 1.67 (dd, J = 37.1, 20.3 Hz, 5H), 1.49 (d, J = 6.4 Hz, 3H). |
| 1137 | 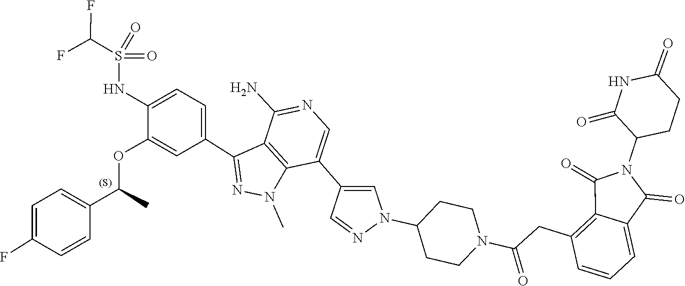 | N-(4-{4-amino-7-[1-(1-{2-[2-(2,6-dioxopiperidin- 3-yl)-1,3-dioxo-2,3-dihydro-1H-isoindol-4- yl]acetyl}piperidin-4-yl)-1H-pyrazol-4-yl]-1- methyl-1H-pyrazolo[4,3-c]pyridin-3-yl}-2- [(1S)-1-(fluorophenyl)ethoxy]phenyl)-1,1- difluoromethanesulfonamide From Intermediate L31. LCMS (Method B): 1.94 min, m/z: 939.3 [M + H]⁺. ¹H NMR (400 MHZ, CDCl₃): 7.66 (d, J = 7.6 Hz, 2H), 7.52 (s, 2H), 7.04 (d, J = 32.8 Hz, 2H), 5.41 (d, J = 57.1 Hz, 2H), 5.30 (s, 5H), 4.95 (s, 1H), 4.30 (s, 1H), 4.14 (d, J = 7.5 Hz, 1H), 3.87 (d, J = 7.4 Hz, 2H), 3.49 (s, 3H, 2.83 (d, J = 36.9 Hz, 2H), 2.62 (s, 1H), 2.34 (s, 2H), 2.26-2.10 (m, 3H), 2.04 (s, 3H), 1.73 (s, 3H), 0.87 (d, J = 7.4 Hz, 8H), 0.22 (s, 1H), −0.08 (s, 1H). |
| 1142 | 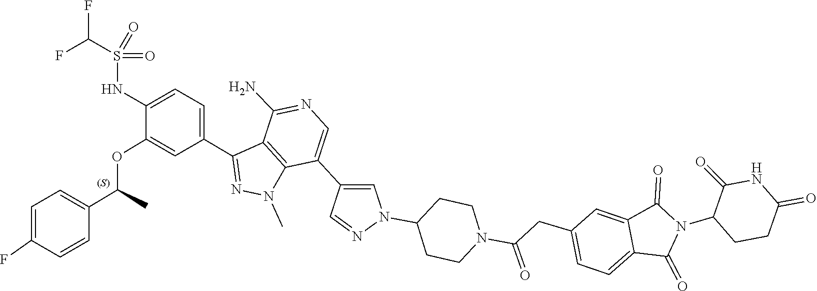 | N-(4-{4-amino-7-[1-(1-{2-[2-(2,6-dioxopiperidin-3- yl)-1,3-dioxo-2,3-dihydro-1H-isoindol-5- yl]acetyl}piperidin-4-yl)-1H-pyrazol-4-yl]-1- methyl-1H-pyrazolo[4,3-c]pyridin-3-yl}-2- [(1S)-1-(4-fluorophenyl)ethoxy]phenyl)-1,1- difluoromethanesulfonamide From Intermediate L32. LCMS (Method B): 1.88 min, m/z: 939.3 [M + H]⁺. ¹H NMR (400 MHZ, DMSO-d₆): 11.15 (s, 1H), 8.09 (d, J = 14.9 Hz, 1H), 8.04-7.79 (m, 3H), 7.68 (s, 1H), 7.65-7.50 (m, 3H), 7.43 (d, J= 8.1 Hz, 1H), 7.16 (q, J = 10.3 Hz, 4H), 6.97 (d, J = 52.6 Hz, 1H), 6.64 (s, 2H), 5.65 (d, J = 6.9 Hz, 1H), 5.18 (d, J = 12.6 Hz, 1H), 4.58 (s, 2H), 4.09 (d, J = 38.6 Hz, 1H), 3.72 (s, 3H), 3.60 (d, J = 14.6 Hz, 1H), 3.08 (s, 1H), 2.89 (s, 1H), 2.61 (d, J = 17.8 Hz, 3H), 2.14 (d, J = 65.7 Hz, 6H), 1.59 (d, J = 6.2 Hz, 3H). |

### Compound 1088: N-[4-(4-amino-7-{1-[1-(3-{1-[2-(2,6-dioxopiperidin-3-yl)-1,3-dioxo-2,3-dihydro-1H-isoindol-4-yl]piperidin-4-yl}propanoyl)piperidin-4-yl]-1H-pyrazol-4-yl}-1-methyl-1H-pyrazolo[4,3-c]pyridin-3-yl}-2-fluorophenyl]ethane-1-sulfonamide

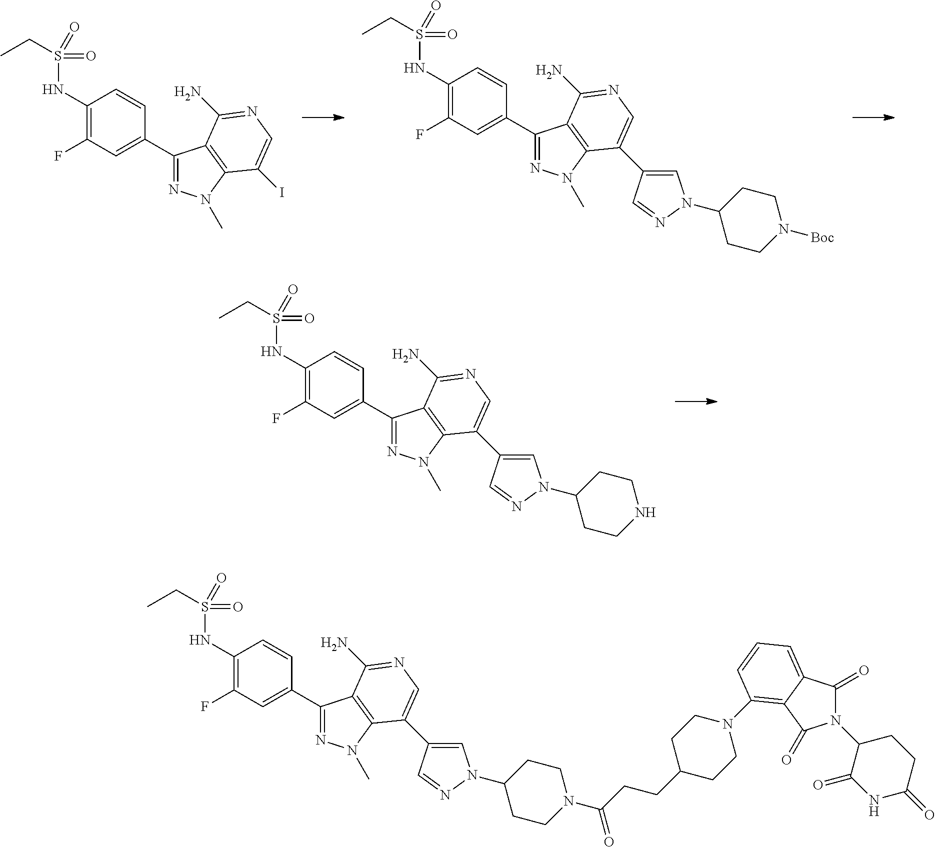 [SMILES: CCS(=O)(=O)Nc1ccc(-c2nn(C)c3c(I)cnc(N)c23)cc1F ; CCS(=O)(=O)Nc1ccc(-c2nn(C)c3c(-c4cnn(C5CCN(C(=O)OC(C)(C)C)CC5)c4)cnc(N)c23)cc1F ; CCS(=O)(=O)Nc1ccc(-c2nn(C)c3c(-c4cnn(C5CCNCC5)c4)cnc(N)c23)cc1F ; CCS(=O)(=O)Nc1ccc(-c2nn(C)c3c(-c4cnn(C5CCN(C(=O)CCC6CCN(c7cccc8c7C(=O)N(C7CCC(=O)NC7=O)C8=O)CC6)CC5)c4)cnc(N)c23)cc1F]

### Step 1: tert-butyl 4-{4-[4-amino-3-(4-ethanesulfonamido-3-fluorophenyl)-1-methyl-1H-pyrazolo [4,3-c]pyridin-7-yl]-1H-pyrazol-1-yl}piperidine-1-carboxylate

A mixture of N-(4-{4-amino-7-iodo-1-methyl-1H-pyrazolo[4,3-c]pyridin-3-yl}-2-fluorophenyl) ethane-1-sulfonamide (466 mg, 980 μmol), tert-butyl 4-[4-(4,4,5,5-tetramethyl-1,3,2-dioxaborolan-2-yl)-1H-pyrazol-1-yl]piperidine-1-carboxylate (443.8 mg, 1.17 mmol), Na₂CO₃ (311.9 mg, 2.93 mmol) and Pd(dppf)Cl₂ (71.2 mg, 98.1 μmol) in 80% aq. 1,4-dioxane (7.5 mL) was stirred at 100° C. for 2 h. The mixture was concentrated, and the residue was purified by column chromatography (DCM/MeOH=80/1) and reverse-phase chromatography to afford the title compound (490 mg, 23%) as a brown solid. LCMS (Method C): 2.63 min, m/z: 599.3 [M+H]⁺.

### Step 2: N-(4-{4-amino-1-methyl-7-[1-(piperidin-4-yl)-1H-pyrazol-4-yl]-1H-pyrazolo[4,3-c]pyridin-3-yl}-2-fluorophenyl)ethane-1-sulfonamide

To a solution of tert-butyl 4-{4-[4-amino-3-(4-ethanesulfonamido-3-fluorophenyl)-1-methyl-1H-pyrazolo[4,3-c]pyridin-7-yl]-1H-pyrazol-1-yl}piperidine-1-carboxylate (0.36 g, 601 μmol) in dioxane (10 mL) was added 4M HCl in 1,4-Dioxane (2.5 mL), and the reaction was stirred at RT overnight. The mixture was concentrated to afford the title compound (0.4 g, >100%) as a brown solid. LCMS (Method C): 0.46 min, m/z: 499.8 [M+H]⁺.

### Step 3: N-[4-(4-amino-7-{1-[1-(3-{1-[2-(2,6-dioxopiperidin-3-yl)-1,3-dioxo-2,3-dihydro-1H-isoindol-4-yl]piperidin-4-yl}propanoyl)piperidin-4-yl]-1H-pyrazol-4-yl}-1-methyl-1H-pyrazolo[4,3-c]pyridin-3-yl}-2-fluorophenyl]ethane-1-sulfonamide

A mixture of N-(4-{4-amino-1-methyl-7-[1-(piperidin-4-yl)-1H-pyrazol-4-yl]-1H-pyrazolo[4,3-c]pyridin-3-yl}-2-fluorophenyl)ethane-1-sulfonamide (162 mg, 326 μmol), 3-{1-[2-(2,6-dioxo piperidin-3-yl)-1,3-dioxo-2,3-dihydro-1H-isoindol-4-yl}piperidin-4-yl)propanoic acid (135 mg, 326 μmol), HATU (185 mg, 489 μmol) and Et₃N (131 mg, 1.30 mmol) in DMF (3 mL) and MeCN (4 mL) was stirred at RT overnight. The mixture was concentrated, and the residue was poured into water (250 mL) and extracted with DCM (200 mL×2). The combined organic layers were washed with sat. aq. NaHCO₃, dried (Na₂SO₄) and concentrated. The residue was purified by Prep-TLC (DCM/MeOH=20/1) to afford the title compound (66.1 mg, 17%) as a yellow solid. LCMS (Method C): 2.59 min, m/z: 894.3 [M+H]⁺. ¹H NMR (400 MHz, DMSO-d₆): 11.14 (s, 1H), 9.90 (s, 1H), 8.19 (s, 1H), 8.10 (s, 1H), 7.73 (dd, J=8.5, 7.1 Hz, 1H), 7.70 (s, 1H), 7.63 (s, 2H), 7.57 (dd, J=11.1, 1.9 Hz, 1H), 7.52 (dd, J=8.3, 1.9 Hz, 1H), 7.39 (s, 2H), 6.16 (s, 2H), 5.18-5.12 (m, 1H), 4.57 (d, J=12.4 Hz, 2H), 4.09 (d, J=13.3 Hz, 1H), 3.80 (s, 3H), 3.76 (d, J=12.1 Hz, 2H), 3.27-3.21 (m, 3H), 2.90 (t, J=11.4 Hz, 3H), 2.81 (d, J=12.3 Hz, 1H), 2.71-2.60 (m, 2H), 2.49 (t, J=7.6 Hz, 2H), 2.18 (t, J=15.8 Hz, 2H), 2.12-1.95 (m, 3H), 1.87 (d, J=12.0 Hz, 3H), 1.58 (t, J=7.2 Hz, 2H), 1.51 (s, 1H), 1.42 (d, J=11.5 Hz, 2H), 1.35 (t, J=7.3 Hz, 3H).

### Compound 1102: N-{4-[4-amino-1-methyl-7-(1-{1-[(trans)-3-{[2-(2,6-dioxopiperidin-3-yl)-1,3-dioxo-2,3-dihydro-1H-isoindol-4-yl]amino}cyclobutanecarbonyl]piperidin-4-yl}-1H-pyrazol-4-yl)-1H-pyrazolo[4,3-c]pyridin-3-yl]-2-fluorophenyl}ethane-1-sulfonamide

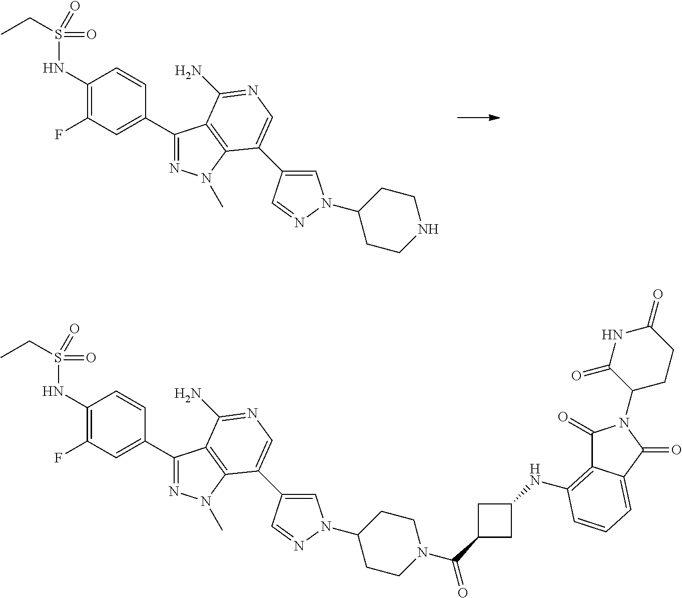 [SMILES: CCS(=O)(=O)Nc1ccc(-c2nn(C)c3c(-c4cnn(C5CCNCC5)c4)cnc(N)c23)cc1F ; CCS(=O)(=O)Nc1ccc(-c2nn(C)c3c(-c4cnn(C5CCN(C(=O)[C@H]6C[C@H](Nc7cccc8c7C(=O)N(C7CCC(=O)NC7=O)C8=O)C6)CC5)c4)cnc(N)c23)cc1F]

A mixture of (trans)-3-{[2-(2,6-dioxopiperidin-3-yl)-1,3-dioxo-2,3-dihydro-1H-isoindol-4-yl]amino}cyclobutane-1-carboxylic acid (53 mg, 142 μmol), N-(4-{4-amino-1-methyl-7-[1-(piperidin-4-yl)-1H-pyrazol-4-yl]-1H-pyrazolo[4,3-c]pyridin-3-yl}-2-fluorophenyl)ethane-1-sulfonamide (70.7 mg, 142 μmol), HATU (80.6 mg, 212 μmol) and Et₃N (57.4 mg, 568 μmol) in DMF (2 mL) was stirred at RT under N₂ overnight. The mixture was concentrated, and the residue was poured into water (250 mL) and extracted with DCM (200 mL×2). The combined organic layers were washed with sat. aq. NaHCO₃, dried (Na₂SO₄) and concentrated. The residue was purified by Prep-TLC (DCM/MeOH=15/1) to afford the title compound (40 mg, 33%) as a yellow solid. LCMS (Method C): 2.44 min, m/z: 852.3 [M+H]⁺. ¹H NMR (400 MHz, DMSO-d₆): 11.10 (s, 1H), 9.74 (s, 2H), 8.03 (s, 1H), 7.64-7.53 (m, 4H), 7.51-7.42 (m, 2H), 7.08 (d, J=7.1 Hz, 1H), 6.92 (d, J=8.5 Hz, 1H), 6.59 (d, J=6.2 Hz, 1H), 5.87 (s, 2H), 5.07 (dd, J=12.8, 5.4 Hz, 1H), 4.51 (t, J=15.6 Hz, 2H), 4.09 (q, J=6.8 Hz, 1H), 3.80 (d, J=13.4 Hz, 1H), 3.52-3.43 (m, 1H), 3.17 (d, J=7.3 Hz, 3H), 2.95-2.76 (m, 3H), 2.75-2.60 (m, 3H), 2.25 (s, 3H), 2.11 (d, J=12.2 Hz, 2H), 2.06-1.96 (m, 3H), 1.93-1.78 (m, 3H), 1.29 (t, J=7.3 Hz, 4H).

### Compound 1113: N-[4-(4-amino-7-{1-[1-(3-{1-[2-(2,6-dioxopiperidin-3-yl)-1,3-dioxo-2,3-dihydro-1H-isoindol-4-yl]piperidin-4-yl}propanoyl)piperidin-4-yl]-1H-pyrazol-4-yl}-1-methyl-1H-pyrazolo[4,3-c]pyridin-3-yl}-2-fluorophenyl]-1,1-difluoromethanesulfonamide

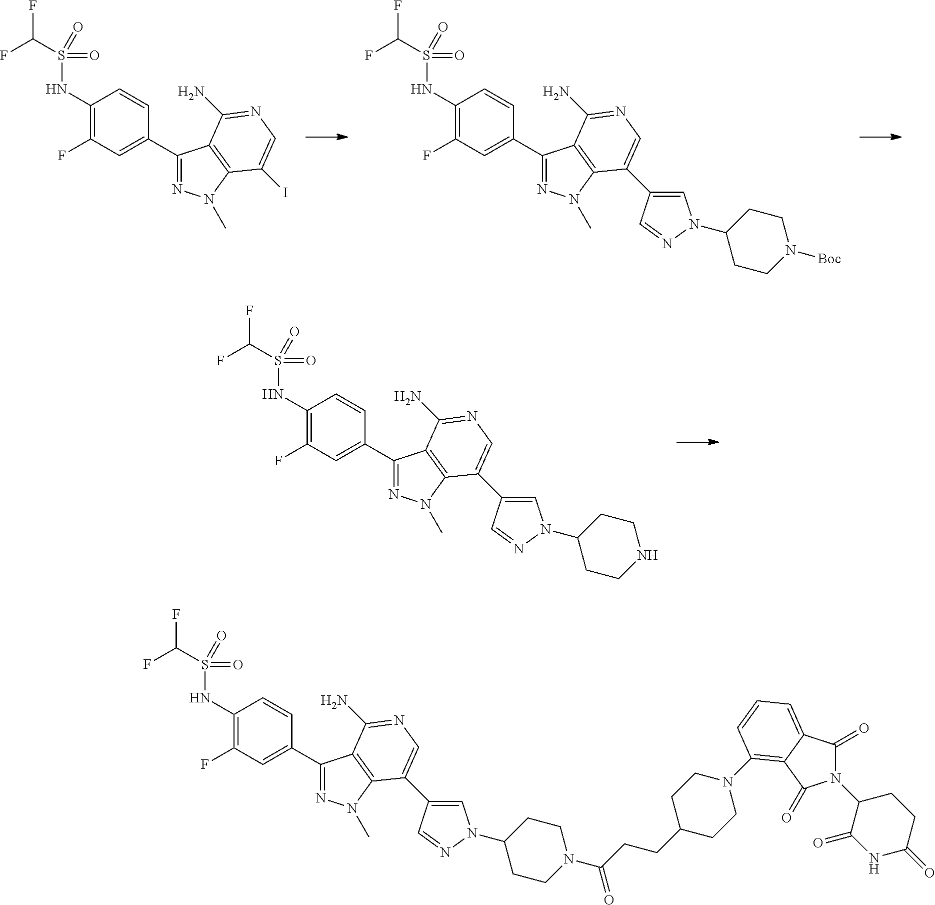 [SMILES: Cn1nc(-c2ccc(NS(=O)(=O)C(F)F)c(F)c2)c2c(N)ncc(I)c21 ; Cn1nc(-c2ccc(NS(=O)(=O)C(F)F)c(F)c2)c2c(N)ncc(-c3cnn(C4CCN(C(=O)OC(C)(C)C)CC4)c3)c21 ; Cn1nc(-c2ccc(NS(=O)(=O)C(F)F)c(F)c2)c2c(N)ncc(-c3cnn(C4CCNCC4)c3)c21 ; Cn1nc(-c2ccc(NS(=O)(=O)C(F)F)c(F)c2)c2c(N)ncc(-c3cnn(C4CCN(C(=O)CCC5CCN(c6cccc7c6C(=O)N(C6CCC(=O)NC6=O)C7=O)CC5)CC4)c3)c21]

### Step 1: tert-butyl 4-(4-{4-amino-3-[4-(difluoromethanesulfonamido)-3-fluorophenyl]-1-methyl-1H-pyrazolo[4,3-c]pyridin-7-yl}-1H-pyrazol-1-yl)piperidine-1-carboxylate

A mixture of N-(4-{4-amino-7-iodo-1-methyl-1H-pyrazolo[4,3-c]pyridin-3-yl}-2-fluorophenyl)-1,1-difluoromethanesulfonamide (1.0 g, 2.01 mmol), tert-butyl 4-[4-(4,4,5,5-tetramethyl-1,3,2-dioxa borolan-2-yl)-1H-pyrazol-1-yl]piperidine-1-carboxylate (0.91 g, 2.41 mmol), Na₂CO₃ (639.58 mg, 6.02 mmol) and Pd(dppf)Cl₂ (1.64 g, 2.01 mmol) in dioxane:H₂O (5:1, 12 mL) was stirred at 100° C. overnight. The mixture was concentrated, and the residue was poured into water and extracted with EtOAc. The combined organic phases were dried over Na₂SO₄ and concentrated. The residue was purified by prep-TLC (DCM:MeOH=20:1) to give the title product (357.1 mg, 29%) as a yellow solid. LCMS (Method A): 1.64 min, m/z: 621.3 [M+H]⁺.

### Step 2: N-(4-{4-amino-1-methyl-7-[1-(piperidin-4-yl)-1H-pyrazol-4-yl]-1H-pyrazolo[4,3-c]pyridin-3-yl}-2-fluorophenyl)-1,1-difluoromethanesulfonamide

A solution of tert-butyl 4-(4-{4-amino-3-[4-(difluoromethanesulfonamido)-3-fluorophenyl]-1-methyl-1H-pyrazolo[4,3-c]pyridin-7-yl}-1H-pyrazol-1-yl)piperidine-1-carboxylate (150 mg, 241 μmol) in 4 M HCl/dioxane (2 mL) was stirred at RT for 2 h. The mixture was concentrated to give the title product (162.3 mg, >100%) as a yellow solid. LCMS (method A): 0.67 min, m/z: 521.2 [M+H]⁺.

### Step 3: N-[4-(4-amino-7-{1-[1-(3-{1-[2-(2,6-dioxopiperidin-3-yl)-1,3-dioxo-2,3-dihydro-1H-isoindol-4-yl]piperidin-4-yl}propanoyl)piperidin-4-yl]-1H-pyrazol-4-yl}-1-methyl-1H-pyrazolo[4,3-c]pyridin-3-yl)-2-fluorophenyl]-1,1-difluoromethanesulfonamide

A mixture of N-(4-{4-amino-1-methyl-7-[1-(piperidin-4-yl)-1H-pyrazol-4-yl]-1H-pyrazolo[4,3-c]pyridin-3-yl}-2-fluorophenyl)-1,1-difluoromethanesulfonamide (50 mg, 96.0 μmol), 3-{1-[2-(2,6-dioxopiperidin-3-yl)-1,3-dioxo-2,3-dihydro-1H-isoindol-4-yl]piperidin-4-yl}propanoic acid (47.69 mg, 115 μmol), HATU (73.09 mg, 191 μmol) and DIPEA (99.38 mg, 768 μmol) in DMF (3 mL) was stirred at RT for 12 h. The mixture was concentrated, then the residue was poured into water and extracted with DCM:MeOH (10:1, 50 mL×3). The combined organic phases were dried over Na₂SO₄ and concentrated. The residue was purified by prep-TLC (DCM:MeOH=10:1) to give the title product (16.2 mg, 18%) as a yellow solid. LCMS (method A): 2.68 min, m/z: 458.8 [M+2H]²⁺. ¹H NMR (400 MHz, DMSO-d₆): 11.08 (s, 1H), 8.09 (s, 1H), 7.70-7.64 (m, 2H), 7.60 (s, 1H), 7.51 (t, J=8.5 Hz, 1H), 7.32 (t, J=7.8 Hz, 4H), 6.71 (d, J=29.7 Hz, 3H), 5.08 (dd, J=12.8, 5.4 Hz, 1H), 4.56-4.45 (m, 2H), 4.03 (d, J=13.5 Hz, 1H), 3.74 (s, 3H), 3.69 (d, J=12.0 Hz, 2H), 3.20 (dd, J=19.8, 7.6 Hz, 2H), 2.89-2.79 (m, 4H), 2.60 (d, J=3.3 Hz, 1H), 2.57 (d, J=3.7 Hz, 1H), 2.53 (d, J=2.1 Hz, 2H), 2.43 (t, J=7.5 Hz, 1H), 2.11 (t, J=16.0 Hz, 2H), 2.06-1.98 (m, 1H), 1.86-1.75 (m, 3H), 1.53 (q, J=6.8 Hz, 3H), 1.42-1.31 (m, 2H).

### Compound 1114: N-{4-[4-amino-1-methyl-7-(1-{1-[(trans)-3-{[2-(2,6-dioxopiperidin-3-yl)-1,3-dioxo-2,3-dihydro-1H-isoindol-4-yl]amino}cyclobutanecarbonyl]piperidin-4-yl}-1H-pyrazol-4-yl)-1H-pyrazolo[4,3-c]pyridin-3-yl]-2-fluorophenyl}-1,1-difluoromethanesulfonamide

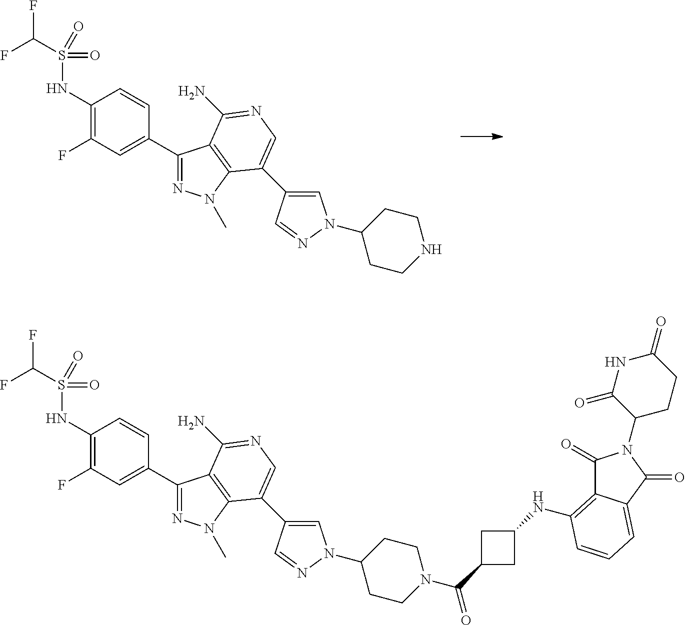 [SMILES: Cn1nc(-c2ccc(NS(=O)(=O)C(F)F)c(F)c2)c2c(N)ncc(-c3cnn(C4CCNCC4)c3)c21 ; Cn1nc(-c2ccc(NS(=O)(=O)C(F)F)c(F)c2)c2c(N)ncc(-c3cnn(C4CCN(C(=O)[C@H]5C[C@H](Nc6cccc7c6C(=O)N(C6CCC(=O)NC6=O)C7=O)C5)CC4)c3)c21]

### Step 1: N-{4-[4-amino-1-methyl-7-(1-{1-[(1r,3r)-3-{[2-(2,6-dioxopiperidin-3-yl)-1,3-dioxo-2,3-dihydro-1H-isoindol-4-yl]amino}cyclobutanecarbonyl]piperidin-4-yl}-1H-pyrazol-4-yl)-1H-pyrazolo [4,3-c]pyridin-3-yl]-2-fluorophenyl}-1,1-difluoromethanesulfonamide

A mixture of N-(4-{4-amino-1-methyl-7-[1-(piperidin-4-yl)-1H-pyrazol-4-yl]-1H-pyrazolo[4,3-c]pyridin-3-yl}-2-fluorophenyl)-1,1-difluoromethanesulfonamide (50 mg, 96.0 μmol), (trans)-3-{[2-(2,6-dioxopiperidin-3-yl)-1,3-dioxo-2,3-dihydro-1H-isoindol-4-yl]amino}cyclobutane-1-carboxylic acid (53.5 mg, 144 μmol), HATU (43.86 mg, 115 μmol) and DIPEA (99.38 mg, 768 μmol) in DMF (3 mL) was stirred at RT for 12 h. The mixture was concentrated, then the residue was diluted with water and extracted with DCM:MeOH (10:1, 50 mL×3). The combine organic phases were dried over Na₂SO₄ and concentrated. The residue was purified by prep-TLC (DCM:MeOH=10:1) to give the title product (8.3 mg, 10%) as a yellow solid. LCMS (method A): 1.68 min, 874.4 [M+H]⁺. ¹H NMR (400 MHz, DMSO-d₆): 7.98 (s, 1H), 7.66-7.33 (m, 4H), 7.07 (d, J=25.3 Hz, 3H), 6.87 (d, J=9.5 Hz, 1H), 6.54 (d, J=7.6 Hz, 1H), 6.23 (t, J=54.9 Hz, 1H), 5.71 (s, 2H), 4.98 (s, 1H), 4.47 (s, 2H), 4.05 (s, 2H), 3.76 (d, J=13.2 Hz, 1H), 3.66 (s, 3H), 3.44 (s, 2H), 3.12 (s, 2H), 2.82 (d, J=7.9 Hz, 2H), 2.21 (s, 3H), 2.06 (s, 2H), 1.82 (s, 2H).

### Compound 1107: N-{4-[4-amino-1-methyl-7-(1-{4-[(1r,3r)-3-{[2-(2,6-dioxopiperidin-3-yl)-1,3-dioxo-2,3-dihydro-1H-isoindol-4-yl]amino}cyclobutanecarbonyl]-4-azaspiro[2.5]octan-7-yl}-1H-pyrazol-4-yl)-1H-pyrazolo[4,3-c]pyridin-3-yl]-2-[(1S)-1-(4-fluorophenyl)ethoxy]phenyl}-1,1-difluoromethanesulfonamide

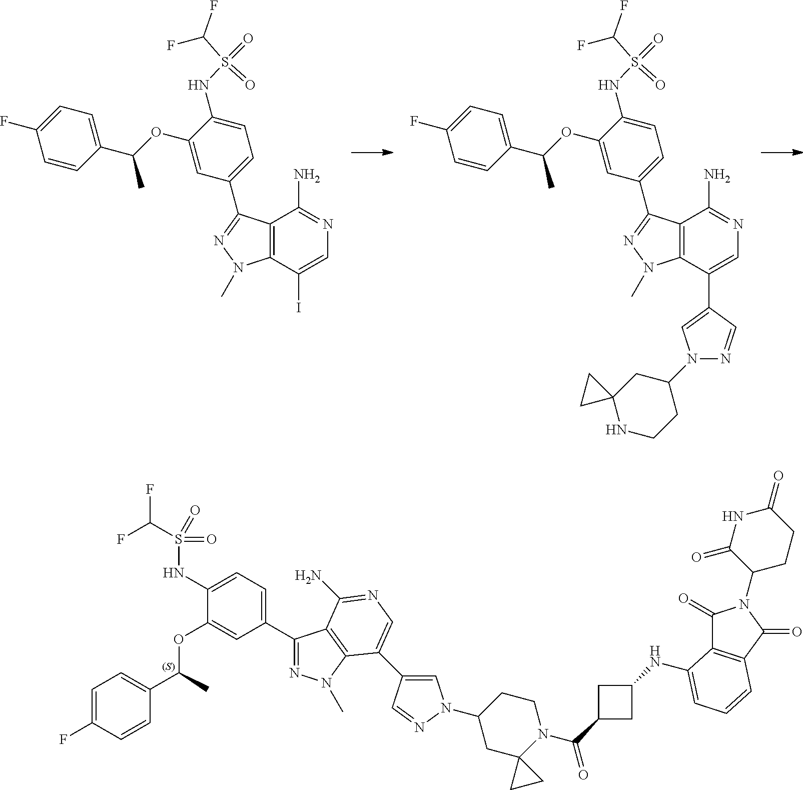

### Step 1: N-{4-[4-amino-7-(1-{4-azaspiro[2.5]octan-7-yl}-1H-pyrazol-4-yl)-1-methyl-1H-pyrazolo [4,3-c]pyridin-3-yl]-2-[(1 S)-1-(4-fluorophenyl)ethoxy]phenyl}-1,1-difluoromethanesulfonamide

The title compound was prepared from Intermediate A1B1 and Intermediate D2, Step 4, following General Procedure C1. The compound was obtained as a yellow solid (200 mg, 60%). LCMS (method A): 1.63 min, m/z: 667.3 [M+H]⁺.

### Step 2: N-{4-[4-amino-1-methyl-7-(1-{4-[(1r,3r)-3-{[2-(2,6-dioxopiperidin-3-yl)-1,3-dioxo-2,3-dihydro-1H-isoindol-4-yl]amino}cyclobutanecarbonyl]-4-azaspiro[2.5]octan-7-yl}-1H-pyrazol-4-yl)-1H-pyrazolo[4,3-c]pyridin-3-yl]-2-[(1 S)-1-(4-fluorophenyl)ethoxy]phenyl}-1,1-difluoromethanesulfonamide

To a mixture of (trans)-3-{[2-(2,6-dioxopiperidin-3-yl)-1,3-dioxo-2,3-dihydro-1H-isoindol-4-yl]amino}cyclobutane-1-carboxylic acid (55.3 mg, 149 μmol), HATU (56.6 mg, 149 μmol) and DIPEA (57.7 mg, 447 μmol) in DMF (5 mL) was added N-{4-[4-amino-7-(1-{4-azaspiro[2.5]octan-7-yl}-1H-pyrazol-4-yl)-1-methyl-1H-pyrazolo[4,3-c]pyridin-3-yl]-2-[(1S)-1-(4-fluorophenyl)ethoxy]phenyl}-1,1-difluoromethanesulfonamide (100 mg, 149 μmol), and the mixture was stirred at RT for 2 h. The mixture was concentrated, and the residue was purified by Prep-TLC (DCM/MeOH=20/1) to give the title compound (30.0 mg, 20%) as a yellow solid. LCMS (method A): 1.91 min, m/z: 1020.4 [M+H]⁺. ¹HNMR (400 MHz, DMSO-d₆): 11.11 (s, 1H), 8.01 (s, 1H), 7.59-7.50 (m, 5H), 7.36 (d, J=8 Hz, 1H), 7.14 (t, J=8.8 Hz, 2H), 7.08 (d, J=7.2 Hz, 1H), 7.01 (d, J=8.4 Hz, 1H), 6.96 (s, 1H), 6.91 (d, J=8.8 Hz, 1H), 6.66 (t, J=54.4 Hz, 1H), 6.58 (d, J=6 Hz, 1H), 5.66-5.63 (m, 3H), 5.06 (dd, J=4.2 Hz, 12.8 Hz, 1H), 4.66-4.49 (m, 2H), 4.24-4.21 (m, 1H), 3.75 (s, 1H), 3.66 (s, 3H), 2.90-2.85 (m, 2H), 2.61-2.57 (m, 2H), 2.35-1.82 (m, 8H), 1.53 (d, J=6.4 Hz, 3H), 0.98-0.64 (m, 4H)

### Compound 1116: N-{4-[4-amino-7-(1-{2,2-dimethyl-1-[(trans)-3-{[2-(2,6-dioxopiperidin-3-yl)-1,3-dioxo-2,3-dihydro-1H-isoindol-4-yl]amino}cyclobutanecarbonyl]piperidin-4-yl}-1H-pyrazol-4-yl)-1-methyl-1H-pyrazolo[4,3-c]pyridin-3-yl]-2-[(1S)-1-(4-fluorophenyl)ethoxy]phenyl}-1,1-difluoromethanesulfonamide

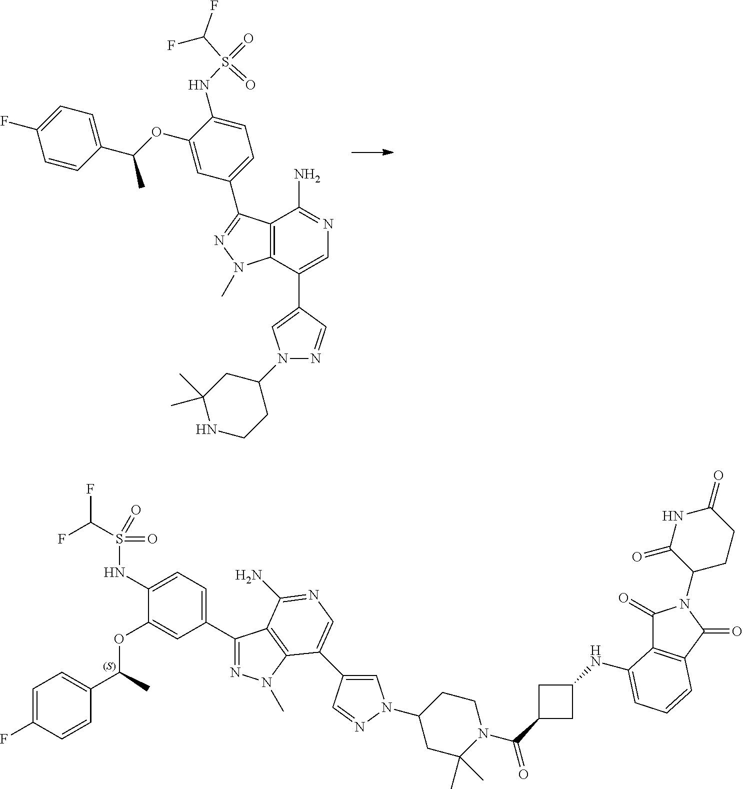

### Step 1: N-{4-[4-amino-7-(1-{2,2-dimethyl-1-[(trans)-3-{[2-(2,6-dioxopiperidin-3-yl)-1,3-dioxo-2,3-dihydro-1H-isoindol-4-yl]amino}cyclobutanecarbonyl]piperidin-4-yl}-1H-pyrazol-4-yl)-1-methyl-1H-pyrazolo[4,3-c]pyridin-3-yl]-2-[(1 S)-1-(4-fluorophenyl)ethoxy]phenyl}-1,1-difluoromethanesulfonamide

To a solution of N-(4-{4-amino-7-[1-(2,2-dimethylpiperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide (37 mg, 55.3 μmol), (trans)-3-{[2-(2,6-dioxopiperidin-3-yl)-1,3-dioxo-2,3-dihydro-1H-isoindol-4-yl]amino}cyclobutane-1-carboxylic acid (20.5 mg, 55.3 μmol) and NMI (15.8 mg, 0.19 mmol) in MeCN (2 mL) was stirred at RT for 20 min before TCFH (23.2 mg, 83.0 μmol) was added. The reaction was stirred at RT overnight, then diluted with water and extracted with EtOAc (3×30 mL). The combined organic layers were washed with brine, dried (Na₂SO₄) and concentrated. The residue was purified by prep-TLC (DCM:MeOH=30:1) to afford the title product (11 mg, 19%) as a yellow solid. LCMS (Method A): 1.84 min; m/z: 511.8 [M+2H]²⁺. ¹H NMR (400 MHz, DMSO-d₆): 11.12 (s, 1H), 10.07 (s, 1H), 8.16 (s, 1H), 7.98 (s, 1H), 7.80 (s, 1H), 7.71 (ddd, J=25.5, 12.4, 6.1 Hz, 2H), 7.54 (dt, J=9.9, 5.0 Hz, 2H), 7.32 (d, J=8.1 Hz, 1H), 7.11 (t, J=8.5 Hz, 2H), 7.00-6.81 (m, 3H), 6.47 (d, J=6.2 Hz, 1H), 6.25 (t, J=55.0 Hz, 1H), 5.64 (d, J=7.1 Hz, 1H), 5.10 (dd, J=12.7, 5.4 Hz, 1H), 4.69 (s, 1H), 4.26 (t, J=6.6 Hz, 1H), 4.01 (d, J=7.2 Hz, 1H), 3.81 (s, 2H), 3.18 (s, 3H), 2.95 (d, J=16.8 Hz, 1H), 2.63 (d, J=17.0 Hz, 2H), 2.31 (s, 2H), 2.18-1.95 (m, 7H), 1.73-1.64 (m, 2H), 1.51 (d, J=6.5 Hz, 3H), 1.46-1.40 (m, 2H), 1.29 (d, J=11.5 Hz, 7H), 0.95 (t, J=7.4 Hz, 2H).

### Compound 1134: N-{4-[4-amino-1-methyl-7-(1-{1-[(trans)-3-{[2-(2,6-dioxopiperidin-3-yl)-1,3-dioxo-2,3-dihydro-1H-isoindol-4-yl]amino}cyclobutanecarbonyl]piperidin-4-yl}-1H-pyrazol-4-yl)-1H-pyrazolo[4,3-c]pyridin-3-yl]-2-fluorophenyl}-2,2,2-trifluoroethane-1-sulfonamide

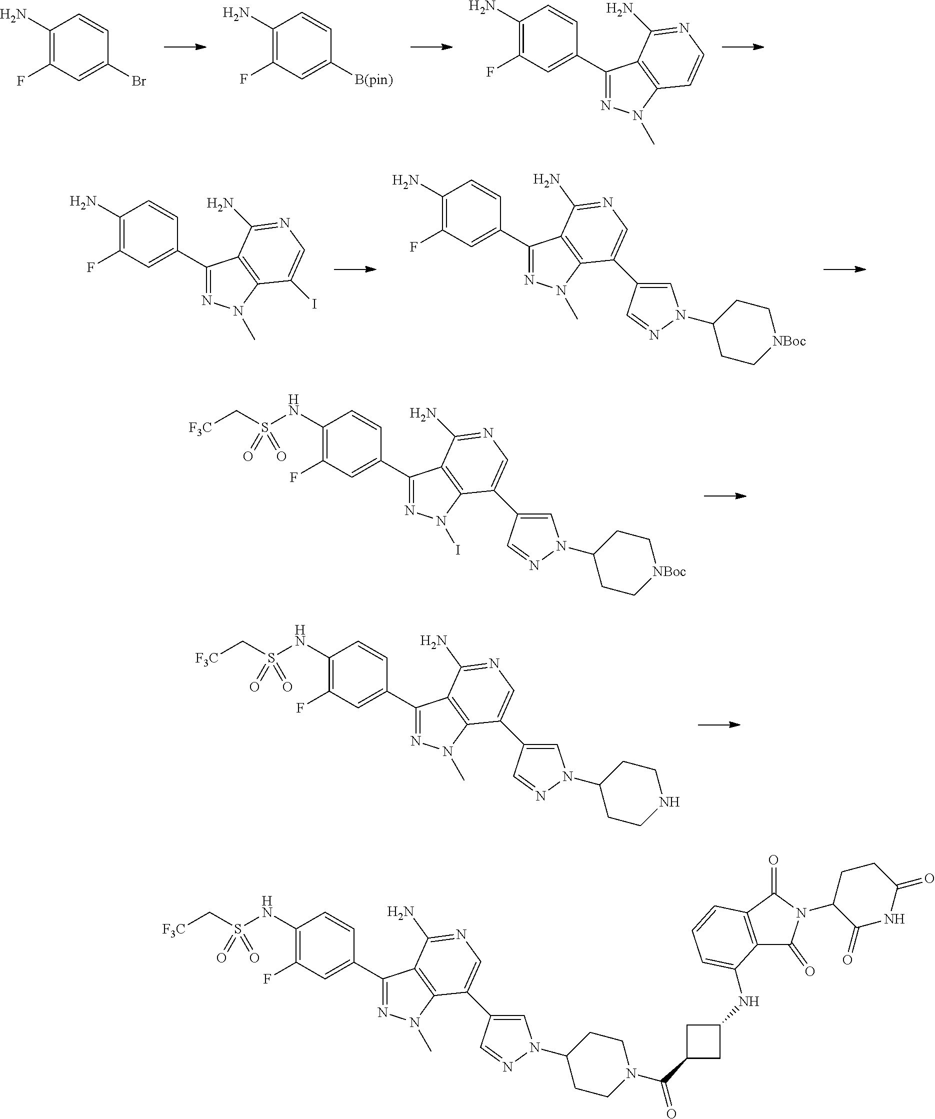

### Step 1: 2-fluoro-4-(4,4,5,5-tetramethyl-1,3,2-dioxaborolan-2-yl)aniline

To a solution of 4-bromo-2-fluoroaniline (2 g, 10.5 mmol) in dioxane (5 mL) were added B₂(pin)₂ (2.66 g, 10.5 mmol), Pd(dppf)Cl₂-DCM (857 mg, 1.05 mmol) and AcOK (2.06 g, 21.0 mmol), and the mixture was stirred at 100° C. overnight. The mixture was concentrated, then the residue was poured into water and extracted with EtOAc. The combined organic phases were dried over Na₂SO₄ and concentrated. The residue was purified by column chromatography (PE:EtOAc=10/1) to give the title product (1.70 g, 69%) as a yellow solid. LCMS (Method A): 2.50 min, m/z: 238.2 [M+H]⁺.

### Step 2: 3-(4-amino-3-fluorophenyl)-1-methyl-1H-pyrazolo[4,3-c]pyridin-4-amine

To a solution of 2-fluoro-4-(4,4,5,5-tetramethyl-1,3,2-dioxaborolan-2-yl)aniline (1.7 g, 7.17 mmol) in dioxane (50 mL) was added 3-iodo-1-methyl-1H-pyrazolo[4,3-c]pyridin-4-amine (1.96 g, 7.17 mmol), Pd(dppf)Cl₂-DCM (585 mg, 717 μmol) and AcOK (1.40 g, 14.3 mmol), and the mixture was stirred at 100° C. overnight. The mixture was concentrated, then the residue was poured into water and extracted with EtOAc. The combined organic phases were dried over Na₂SO₄ and concentrated. The residue was purified by column chromatography (PE:EtOAc=10/1) to give the title product (1.30 g, 71%) as a yellow solid. LCMS (Method A): 1.89 min, m/z 258.1 [M+H]⁺.

### Step 3: 3-(4-amino-3-fluorophenyl)-7-iodo-1-methyl-1H-pyrazolo[4,3-c]pyridin-4-amine

A mixture of 3-(4-amino-3-fluorophenyl)-1-methyl-1H-pyrazolo[4,3-c]pyridin-4-amine (1.4 g, 5.44 mmol) and NIS (1.22 g, 5.44 mmol) in MeCN (5 mL) was stirred at 50° C. overnight. The mixture was concentrated, and the residue was purified by Prep-TLC (DCM/MeOH=10/1) to give the title product (1.20 g, 57%) as a brown solid. LCMS (Method A): 1.31 min. m/z 384.0 [M+H]⁺.

### Step 4: tert-butyl 4-{4-[4-amino-3-(4-amino-3-fluorophenyl)-1-methyl-1H-pyrazolo[4,3-c]pyridin-7-yl]-1H-pyrazol-1-yl}piperidine-1-carboxylate

To a solution of 3-(4-amino-3-fluorophenyl)-7-iodo-1-methyl-1H-pyrazolo[4,3-c]pyridin-4-amine (1.2 g, 3.13 mmol) in dioxane (5 mL) and water (1 mL) were added tert-butyl 4-[4-(4,4,5,5-tetramethyl-1,3,2-dioxaborolan-2-yl)-1H-pyrazol-1-yl]piperidine-1-carboxylate (1.18 g, 3.13 mmol), Pd(dppf)Cl₂-DCM (255 mg, 313 μmol) and Na₂CO₃ (994 mg, 9.38 mmol), and the mixture was stirred at 100° C. overnight. The mixture was concentrated, then the residue was poured into water and extracted with EtOAc. The combined organic phases were dried over Na₂SO₄ and concentrated. The residue was purified by column chromatography (DCM:MeOH=10/1) to give the title product (500 mg, 32%) as a brown solid. LCMS (Method A): 1.74 min, m/z 507.3 [M+H]⁺.

### Step 5: tert-butyl 4-(4-{4-amino-3-[3-fluoro-4-(2,2,2-trifluoroethanesulfonamido)phenyl]-1-methyl-1H-pyrazolo[4,3-c]pyridin-7-yl}-1H-pyrazol-1-yl)piperidine-1-carboxylate

A mixture of 2,2,2-trifluoroethane-1-sulfonyl chloride (179 mg, 985 μmol), tert-butyl 4-{4-[4-amino-3-(4-amino-3-fluorophenyl)-1-methyl-1H-pyrazolo[4,3-c]pyridin-7-yl]-1H-pyrazol-1-yl}piperidine-1-carboxylate (500 mg, 986 μmol) and pyridine (389 mg, 4.93 mmol) in DCM (30 mL) was stirred at RT overnight. The mixture was concentrated, then the residue was poured into water and extracted with EtOAc. The combined organic phases were dried over Na₂SO₄ and concentrated. The residue was purified by column chromatography (PE:EtOAc=10/1) to give the title product (250 mg, 39%) as a brown solid. LCMS (Method A): 2.02 min, m/z 653.3 [M+H]⁺.

### Step 6: N-(4-{4-amino-1-methyl-7-[1-(piperidin-4-yl)-1H-pyrazol-4-yl]-1H-pyrazolo[4,3-c]pyridin-3-yl}-2-fluorophenyl)-2,2,2-trifluoroethane-1-sulfonamide

A solution of tert-butyl 4-(4-{4-amino-3-[3-fluoro-4-(2,2,2-trifluoroethanesulfonamido)phenyl]-1-methyl-1H-pyrazolo[4,3-c]pyridin-7-yl}-1H-pyrazol-1-yl)piperidine-1-carboxylate (250 mg, 383 μmol) in 4 M HCl/dioxane (5 mL) was stirred at RT for 2 h. The mixture was diluted with H₂O (30 mL) and extracted with EtOAc (3×20 mL). The combined organic layers were dried (Na₂SO₄) and concentrated, then the residue was purified by prep-TLC (DCM:MeOH=20:1) to afford the title product (150 mg, 71%) as a brown solid. LCMS (method A): 0.94 min, m/z 553.1 [M+H]⁺.

### Step 7: N-{4-[4-amino-1-methyl-7-(1-{1-[(trans)-3-{[2-(2,6-dioxopiperidin-3-yl)-1,3-dioxo-2,3-dihydro-1H-isoindol-4-yl]amino}cyclobutanecarbonyl]piperidin-4-yl}-1H-pyrazol-4-yl)-1H-pyrazolo[4,3-c]pyridin-3-yl]-2-fluorophenyl}-2,2,2-trifluoroethane-1-sulfonamide

A mixture of N-(4-{4-amino-1-methyl-7-[1-(piperidin-4-yl)-1H-pyrazol-4-yl]-1H-pyrazolo[4,3-c]pyridin-3-yl}-2-fluorophenyl)-2,2,2-trifluoroethane-1-sulfonamide (50 mg, 90.4 μmol), (trans)-3-{[2-(2,6-dioxopiperidin-3-yl)-1,3-dioxo-2,3-dihydro-1H-isoindol-5-yl]amino}cyclobutane-1-carboxylic acid (50.1 mg, 135 μmol), HATU (41.0 mg, 108 μmol) and DIPEA (11.6 mg, 90.4 μmol) in DMF (2 mL) was stirred at RT for 1 h. The mixture was diluted with H₂O (100 mL) and extracted with EtOAc (3×200 mL). The combined organic layers were dried (Na₂SO₄) and concentrated, then the residue was purified by Prep-TLC (DCM:MeOH=10:1) to afford the title product (10.0 mg, 12%) as a yellow solid. LCMS (method A): 1.76 min, m/z 906.3 [M+H]⁺. ¹H NMR (400 MHz, DMSO-d₆): 11.09 (s, 1H), 8.01 (s, 1H), 7.61 (s, 1H), 7.52 (s, 1H), 7.41 (t, J=8.9 Hz, 1H), 7.16-7.05 (m, 3H), 6.92 (d, J=8.6 Hz, 1H), 6.58 (d, J=6.3 Hz, 1H), 5.76 (s, 2H), 5.06 (dd, J=12.8, 5.3 Hz, 1H), 4.52 (d, J=15.8 Hz, 3H), 4.09 (s, 3H), 3.80 (d, J=15.1 Hz, 1H), 3.67 (d, J=19.4 Hz, 4H), 3.48 (s, 2H), 2.93-2.79 (m, 2H), 2.66 (d, J=23.5 Hz, 4H), 2.27 (d, J=12.4 Hz, 3H), 2.12 (s, 4H), 2.05 (s, 1H), 1.87 (s, 2H).

### Synthesis of Intermediates M

### Intermediate M1: 1-[2-(2,6-dioxopiperidin-3-yl)-1,3-dioxo-2,3-dihydro-1H-isoindol-4-yl]pyrrolidine-3-carbaldehyde

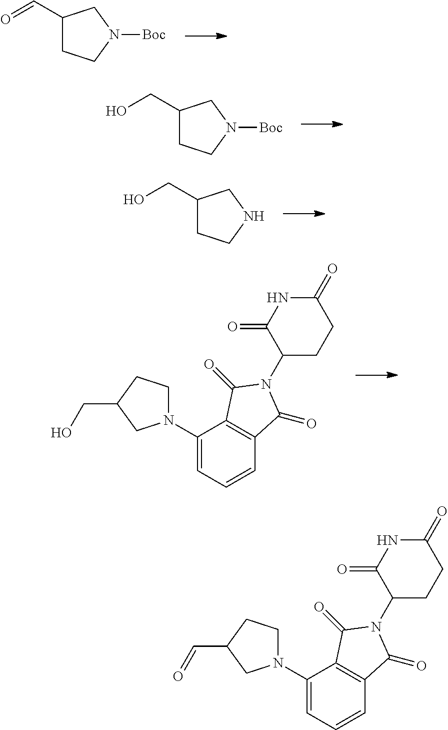

### Step 1: tert-butyl 3-(hydroxymethyl)pyrrolidine-1-carboxylate

To a 0° C. solution of methyl tert-butyl 3-formylpyrrolidine-1-carboxylate (100 mg, 501 μmol) in MeOH (5 mL) was added NaBH₄ (56.7 mg, 1.50 mmol) in portions. The reaction was stirred at 0° C. for 4 h, then adjusted to pH=3-4 with 2M HCl. The precipitate was collected via filtration, washed with water, and dried to afford the title compound (95 mg, 95%) as a white solid. LCMS (Method A): 2.61 min, m/z 224.1 [M+Na]⁺.

### Step 2: (pyrrolidin-3-yl)methanol

A solution of tert-butyl 3-(hydroxymethyl)pyrrolidine-1-carboxylate (100 mg, 688 μmol) in 4M HCl/1,4-dioxane (3 mL) was stirred at RT for 10 mins. The mixture was adjusted to pH=8 with sat. aq. Na₂CO₃ and extracted with EtOAc (50 mL×2). The combined organic phases were washed with water and brine, dried over Na₂SO₄ and concentrated. The residue was purified by column chromatography (DCM/MeOH=30/1) to afford the title compound (43.6 mg, 63%) as a yellow solid. LCMS (Method A): 2.50 min, m/z 224.1 [2M+Na]⁺.

### Step 3: 2-(2,6-dioxopiperidin-3-yl)-4-[3-(hydroxymethyl)pyrrolidin-1-yl]-2,3-dihydro-1H-isoindole-1,3-dione

A mixture of 2-(2,6-dioxopiperidin-3-yl)-4-fluoro-2,3-dihydro-1H-isoindole-1,3-dione (453 mg, 1.64 mmol), (pyrrolidin-3-yl)methanol (200 mg, 1.97 mmol) and DIPEA (635 mg, 4.92 mmol) in DMSO (5 mL) was stirred at 100° C. overnight. Once cooled, the mixture was diluted with MeOH, and adjusted to pH=8 with sat. aq. Na₂CO₃. The mixture was extracted with EtOAc (50 mL×2), and the combined organic phases were washed with water and brine, dried over Na₂SO₄ and concentrated. The residue was purified by prep-TLC (DCM/MeOH=30/1) to afford the title compound (113 mg, 19%) as a yellow solid. LCMS (Method A): 2.46 min, m/z: 358.1 [M+H]⁺.

### Step 4: 1-[2-(2,6-dioxopiperidin-3-yl)-1,3-dioxo-2,3-dihydro-1H-isoindol-4-yl]pyrrolidine-3-carbaldehyde

A mixture of 2-(2,6-dioxopiperidin-3-yl)-4-[3-(hydroxymethyl)pyrrolidin-1-yl]-2,3-dihydro-1H-isoindole-1,3-dione (156 mg, 436 μmol) and DMP (277 mg, 654 μmol) in DCM (5 mL) was stirred at 0° C. for 2 h. The reaction was quenched with MeOH, then the mixture was adjusted to pH=7 with sat. aq. Na₂CO₃ and extracted with EtOAc (50 mL×2). The combined organic phases were washed with water and brine, dried over Na₂SO₄ and concentrated. The residue was purified by prep-TLC (DCM/MeOH=30/1) to afford the title compound (20 mg, 13%) as a yellow solid. LCMS (Method A): 2.43 min, m/z: 356.1 [M+H]⁺. The following examples were prepared according to the procedure for the synthesis of Intermediate M1 utilising the indicated amine in Step 1.

| Inter- | | | LCMS | |
|---|---|---|---|---|
| mediate | Name | Structure | data | SM |
| M2 | 1-[2-(2,6-dioxopiperidin-3-yl)-1,3- dioxo-2,3-dihydro-1H-isoindol-4- yl]piperidine-4-carbaldehyde | 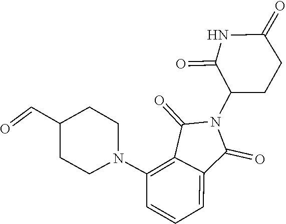 [SMILES: O=CC1CCN(c2cccc3c2C(=O)N(C2CCC(=O)NC2=O)C3=O)CC1] | LCMS (Method A): 2.77 min, m/z: 370.1 [M + H]⁺. | tert-butyl 4- formylpiperidine-1- carboxylate |
| M3 | 2-{1-[2-(2,6-dioxopiperidin-3-yl)- 1,3-dioxo-2,3-dihydro-1H- isoindol-4-yl]piperidin-4-yl}acetaldehyde | 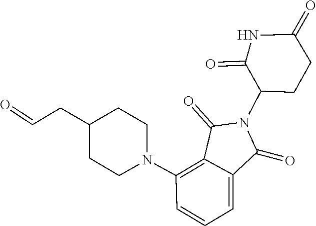 [SMILES: O=CCC1CCN(c2cccc3c2C(=O)N(C2CCC(=O)NC2=O)C3=O)CC1] | LCMS (Method C): 3.31 min, m/z: 384.7 [M + H]⁺. | tert-butyl 4-(2- oxoethyl)piperidine-1- carboxylate |

### Intermediate M4: 3-{1-[2-(2,6-dioxopiperidin-3-yl)-1,3-dioxo-2,3-dihydro-1H-isoindol-4-yl]piperidin-4-yl}propanal

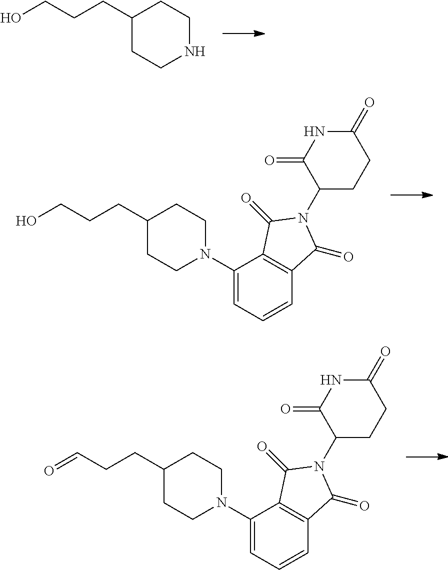 [SMILES: OCCCC1CCNCC1 ; O=C1CCC(N2C(=O)c3cccc(N4CCC(CCCO)CC4)c3C2=O)C(=O)N1 ; O=CCCC1CCN(c2cccc3c2C(=O)N(C2CCC(=O)NC2=O)C3=O)CC1]

### Step 1: 2-(2,6-dioxopiperidin-3-yl)-4-[4-(3-hydroxypropyl)piperidin-1-yl]-2,3-dihydro-1H-isoindole-1,3-dione

A mixture of 3-(piperidin-4-yl)propan-1-ol (100 mg, 698 μmol), 2-(2,6-dioxopiperidin-3-yl)-4-fluoro-2,3-dihydro-1H-isoindole-1,3-dione (174 mg, 629 μmol) and DIPEA (271.2 mg, 2.09 mmol) in DMSO (4 mL) was stirred at 100° C. overnight. Once cooled, the mixture was diluted with brine and extracted with EtOAc. The combined organics were washed with H₂O, dried over Na₂SO₄ and concentrated. The residue was purified by column chromatography to afford the title compound (210 mg, 76%). LCMS (Method C): 3.29 min, m/z: 400.8 [M+H]⁺.

### Step 2: 3-{1-[2-(2,6-dioxopiperidin-3-yl)-1,3-dioxo-2,3-dihydro-1H-isoindol-4-yl]piperidin-4-yl}propanal

A mixture of 2-(2,6-dioxopiperidin-3-yl)-4-[4-(3-hydroxypropyl)piperidin-1-yl]-2,3-dihydro-1H-isoindole-1,3-dione (210 mg, 525 μmol) and DMP (444 mg, 1.04 mmol) in DCM (5 mL) was stirred at RT for 5 h. The mixture was diluted with water and extracted with DCM. The organics were washed with H₂O, dried over Na₂SO₄ and concentrated. The residue was purified by prep-TLC to afford the title compound (200 mg, 96%). LCMS (Method C): 3.07 min, m/z: 398.2 [M+H]⁺.

The following examples were prepared according to the procedure for the synthesis of Intermediate M4 utilising the indicated amine in Step 1.

| Intermediate | Name | Structure | LCMS data | SM |
|---|---|---|---|---|
| M5 | 3-({[2-(2,6-dioxopiperidin-3-yl)- 1,3-dioxo-2,3-dihydro-1H- isoindol-4- yl]amino}methyl)benzaldehyde | 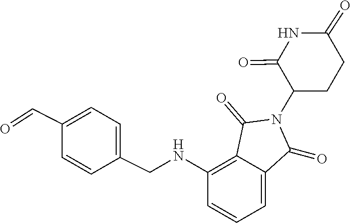 [SMILES: O=Cc1ccc(CNc2cccc3c2C(=O)N(C2CCC(=O)NC2=O)C3=O)cc1] | LCMS (Method B): 3.09 min, m/z: 414.1 [M + Na]⁺. | [3-(aminomethyl) phenyl]methanol |
| M6 | (trans)-3-{[2-(2,6- dioxopiperidin-3-yl)-1,3-dioxo- 2,3-dihydro-1H-isoindol-4- yl]amino}cyclobutane-1- carbaldehyde | 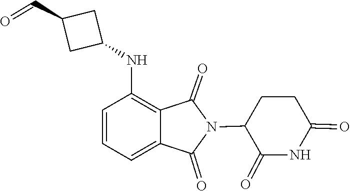 [SMILES: O=C[C@H]1C[C@H](Nc2cccc3c2C(=O)N(C2CCC(=O)NC2=O)C3=O)C1] | LCMS (Method A): 3.20 min, m/z: 356.6 [M + H]⁺ | [(trans)-3- aminocyclobutyl] methanol |

### Intermediate M7: 2-[2-(2,6-dioxopiperidin-3-yl)-1,3-dioxo-2,3-dihydro-1H-isoindol-4-yl]acetaldehyde

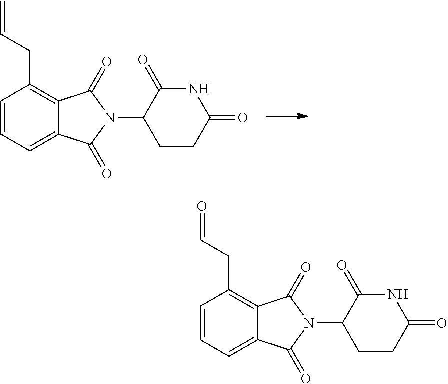 [SMILES: C=CCc1cccc2c1C(=O)N(C1CCC(=O)NC1=O)C2=O ; O=CCc1cccc2c1C(=O)N(C1CCC(=O)NC1=O)C2=O]

### Step 1: 2-[2-(2,6-dioxopiperidin-3-yl)-1,3-dioxo-2,3-dihydro-1H-isoindol-4-yl]acetaldehyde

To a solution of 2-(2,6-dioxopiperidin-3-yl)-4-(prop-2-en-1-yl)-2,3-dihydro-1H-isoindole-1,3-dione (270 mg, 905 μmol) in DCM (2 mL) was added O₃ (10 mL) over 30 min, and the mixture was stirred at −70° C. under O₃ for 1 h. The reaction was quenched with SMe₂ (28.0 mg, 452 μmol), then concentrated to afford the title product (100 mg, 37%) as a brown solid. LCMS (Method B): 1.82 min, m/z: 301.1 [M+H]⁺.

### Intermediate M8: 2-[2-(2,6-dioxopiperidin-3-yl)-1,3-dioxo-2,3-dihydro-1H-isoindol-5-yl]acetaldehyde

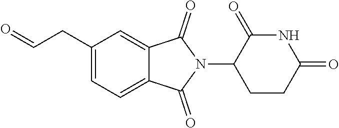 [SMILES: O=CCc1ccc2c(c1)C(=O)N(C1CCC(=O)NC1=O)C2=O]

Intermediate M8 was prepared from 2-(2,6-dioxopiperidin-3-yl)-5-(prop-2-en-1-yl)-2,3-dihydro-1H-isoindole-1,3-dione according to the procedure for the synthesis of Intermediate M7. The title compound was obtained as white solid (84.0 mg, 70%). LCMS (Method A): 1.23 min, m/z 301.1 [M+H]⁺.

### Synthesis of Examples M

### Compound 1083: N-[4-(4-amino-7-{1-[1-({1-[2-(2,6-dioxopiperidin-3-yl)-1,3-dioxo-2,3-dihydro-1H-isoindol-4-yl]pyrrolidin-3-yl}methyl)piperidin-4-yl]-1H-pyrazol-4-yl}-1-methyl-1H-pyrazolo[4,3-c]pyridin-3-yl}-2-[(1S)-1-(4-fluorophenyl)ethoxy]phenyl]-1,1-difluoromethanesulfonamide

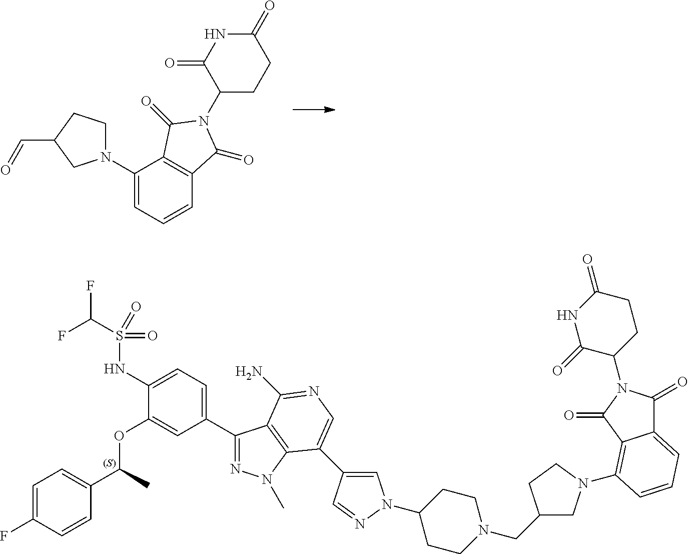

To a mixture of 1-[2-(2,6-dioxopiperidin-3-yl)-1,3-dioxo-2,3-dihydro-1H-isoindol-4-yl]pyrrolidine-3-carbaldehyde (20 mg, 56.2 μmol) and N-(4-{4-amino-1-methyl-7-[1-(piperidin-4-yl)-1H-pyrazol-4-yl]-1H-pyrazolo[4,3-c]pyridin-3-yl}-2-[(1S)-1-(4-fluorophenyl)ethoxy]phenyl)-1,1-difluoromethane sulfonamide (36.0 mg, 56.2 μmol) and AcOH (10.0 mg, 168 μmol), in MeOH (3 mL) was added Na(CN)BH₃ (5.64 mg, 89.9 μmol), and the reaction was stirred at RT for 4 h. Water was added, and the precipitate was collected via filtration, washed with water and dried to afford the title compound (5 mg, 9%) as a yellow solid. LCMS (Method A): 2.37 min, m/z: 490.8 [M+2H]²⁺. ¹H NMR (400 MHz, DMSO-d₆): 11.08 (s, 1H), 8.00 (s, 1H), 7.61 (s, 1H), 7.59-7.52 (m, 4H), 7.40 (d, J=8.0 Hz, 1H), 7.20-7.06 (m, 7H), 6.95-6.80 (m, 1H), 5.71 (s, 2H), 5.65 (d, J=6.4 Hz, 1H), 5.07 (dd, J=12.8, 5.4 Hz, 1H), 4.32-4.20 (m, 1H), 3.67-3.56 (m, 4H), 3.44-3.35 (m, 4H), 3.09 (d, J=14.5 Hz, 3H), 2.94-2.82 (m, 2H), 2.64-2.53 (m, 4H), 2.35-2.22 (m, 2H), 2.16-1.95 (m, 7H), 1.71 (s, 1H), 1.58 (d, J=6.2 Hz, 3H).

The following examples were prepared according to the procedure for the synthesis of Compound 1083 utilising the indicated Intermediate M in Step 1.

TABLE 20
Compounds 1079, 1076, 1078, 1095, 1108, 1136 and 1123

| Example | | |
|---|---|---|
| No. | Structure | Compound Name and Analytical Data |
| 1079 | 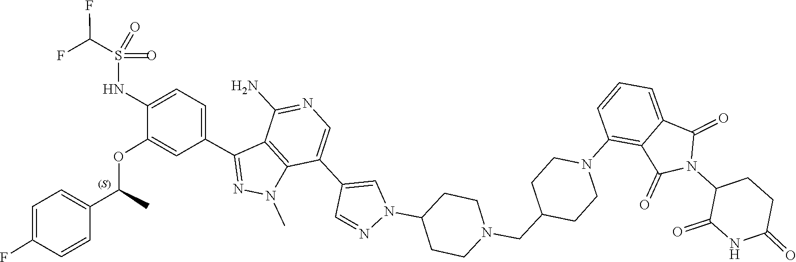 | N-[4-(4-amino-7-{1-[1-({1-[2-(2,6-dioxopiperidin-3- yl)-1,3-dioxo-2,3-dihydro-1H-isoindol-4-yl]piperidin-4- yl}methyl)piperidin-4-yl]-1H-pyrazol-4-yl}-1-methyl -1H-pyrazolo[4,3-c]pyridin-3-yl)-2-[(1S)-1- (4-fluorophenyl)ethoxy]phenyl]-1,1-difluoromethane- sulfonamide From Intermediate M2. LCMS (Method A2): 2.52 min, m/z: 497.8 [M + 2H]²⁺. ¹H NMR (400 MHZ, DMSO-d₆): 11.10 (s, 1H), 8.02 (s, 1H), 7.69 (dd, J = 8.5, 7.1 Hz, 1H), 7.62 (s, 1H), 7.60-7.52 (m, 3H), 7.42-7.30 (m, 3H), 7.15 (t, J = 8.9 Hz, 2H), 7.09-6.97 (m, 2H), 6.75 (t, J = 53.5 Hz, 1H), 5.79-5.62 (m, 3H), 5.10 (dd, J = 12.9, 5.4 Hz, 1H), 4.32 (s, 1H), 3.72 (s, 1H), 3.69 (s, 3H), 2.96- 2.84 (m, 3H), 2.16 (s, 4H), 2.07-2.00 (m, 1H), 1.92 (s, 5H), 1.55 (d, J = 6.3 Hz, 3H), 1.43- 1.34 (m, 2H), 1.24 (d, J = 3.6 Hz, 2H). |
| 1076 | 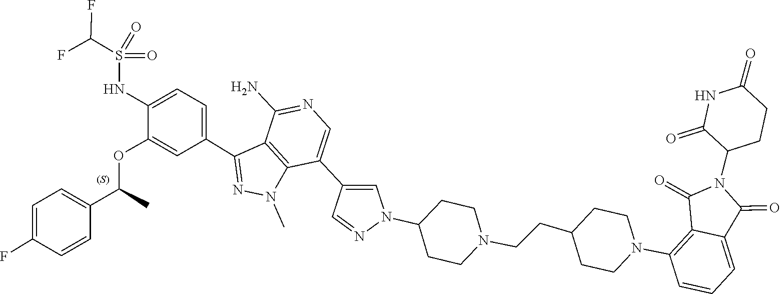 | N-[4-(4-amino-7-{1-[1-(2-{1-[2-(2,6-dioxopiperidin- 3-yl)-1,3-dioxo-2,3-dihydro-1H-isoindol-4- yl]piperidin-4-yl}ethyl)piperidin-4-yl]-1H- pyrazol-4-yl}-1-methyl-1H-pyrazolo[4,3- c]pyridin-3-yl)-2-[(1S)-1-(4-fluorophenyl)ethoxy] phenyl]-1,1-difluoromethane-sulfonamide From Intermediate M3. LCMS (Method A2): 2.73 min, m/z: 1009.2 [M + H]⁺. ¹H NMR (400 MHZ, DMSO-d₆): 11.09 (s, 1H), 8.00 (s, 1H), 7.71-7.65 (m, 1H), 7.61 (s, 1H), 7.56- 7.50 (m, 3H), 7.40-7.29 (m, 3H), 7.15 (t, J = 8.9 Hz, 2H), 7.07-6.98 (m, 2H), 6.82 (d, J = 55.5 Hz, 1H), 5.65 (d, J = 6.1 Hz, 3H), 5.09 (dd, J = 12.8, 5.4 Hz, 1H), 4.29 (s, 1H), 3.68 (s, 5H), 3.17 (s, 3H), 2.86 (t, J = 11.0 Hz, 3H), 2.70-2.54 (m, 4H), 2.15-1.96 (m, 6H), 1.80 (d, J = 12.1 Hz, 2H), 1.58-1.35 (m, 10H). |
| 1078 | 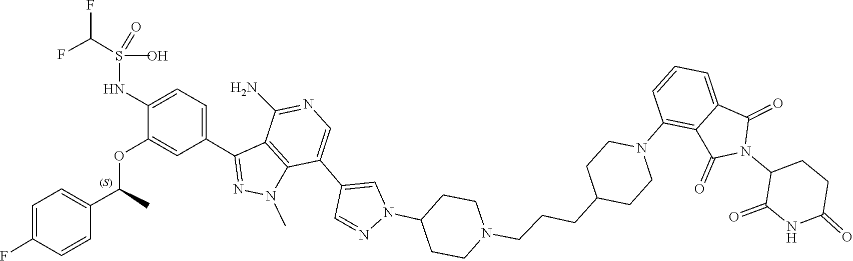 | N-[4-(4-amino-7-{1-[1-(3-{1-[2-(2,6-dioxopiperidin- 3-yl)-1,3-dioxo-2,3-dihydro-1H-isoindol-4- yl]piperidin-4-yl}propyl)piperidin-4-yl]-1H- pyrazol-4-yl}-1-methyl-1H-pyrazolo[4,3- c]pyridin-3-yl)-2-[(1S)-1-(4-fluorophenyl)ethoxy] phenyl]-1,1-difluoromethane-sulfonamide From Intermediate M4. LCMS (Method C): 3.31 min, m/z: 1023.1 [M + H]⁺. ¹H NMR (400 MHZ, DMSO-d₆): 11.09 (s, 1H), 8.00 (s, 1H), 7.68 (dd, J = 8.5, 7.0 Hz, 1H), 7.61 (s, 1H), 7.58-7.48 (m, 3H), 7.38 (d, J = 8.1 Hz, 1H), 7.33 (s, 2H), 7.17 (d, J = 2.1 Hz, 2H), 7.05 (dd, J = 8.1, 1.9 Hz, 1H), 7.01 (d, J = 1.9 Hz, 1H), 6.76 (s, 2H), 5.66 (d, J = 3.7 Hz, 1H), 5.64 (d, J = 6.4 Hz, 1H), 5.09 (dd, J = 12.8, 5.4 Hz, 1H), 4.30 (s, 1H), 3.71 (s, 1H), 3.69 (d, J = 2.5 Hz, 4H), 3.19 (d, J = 12.6 Hz, 4H), 2.90-2.83 (m, 2H), 2.63-2.56 (m, 2H), 2.11 (d, J = 13.6 Hz, 3H), 2.07- 1.93 (m, 2H), 1.79 (d, J = 11.9 Hz, 2H), 1.59 (s, 1H), 1.55 (d, J = 6.3 Hz, 3H), 1.46 (s, 1H), 1.32 (dt, J = 16.1, 4.3 Hz, 3H), 1.24 (d, J = 3.5 Hz, 3H) |
| 1095 | 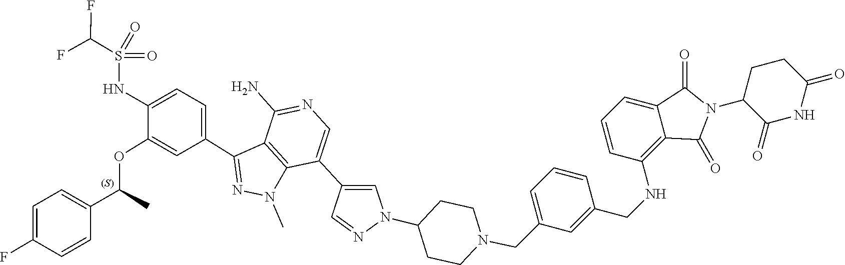 | N-(4-{4-amino-7-[1-(1-{[3-{{[2-(2,6-dioxopiperidin- 3-yl)-1,3-dioxo-2,3-dihydro-1H-isoindol-4- yl]amino}methyl)phenyl]methyl}piperidin-4-yl)-1H- pyrazol-4-yl]-1-methyl-1H-pyrazolo[4,3-c]pyridin-3- yl}-2-[(1S)-1-(4-fluorophenyl)ethoxy]phenyl)-1,1- difluoromethane-sulfonamide From Intermediate M5. LCMS (Method B): 2.67 min, m/z: 508.8 [M + 2H]²⁺. ¹H NMR (400 MHZ, DMSO-d₆): 11.11 (s, 1H), 7.96 (s, 1H), 7.58(s,1H), 7.55 (d, J = 2 Hz, 1H), 7.58 (s, 1H), 7.49 (t, J = 8 Hz, 1H), 7.39 (d, J = 8 Hz, 1H), 7.35 (s, 1H), 7.31 (d, J = 4 Hz), 7.28- 7.26 (m, 1H), 7.24-7.20 (m, 2H), 7.16 (t, J = 10 Hz, 2H), 7.08 (d, J = 8 Hz, 1H), 7.05 (d, J = 4 Hz, 1H), 7.02 (d, J = 4 Hz, 1H), 6.99-8.72 (m, 1H), 6.96 (d, J = 12 Hz), 5.67-5.66 (m, 3H), 5.06 (q, J = 5.3Hz, 1H), 4.57 (d, J = 8 Hz, 2H), 4.22-4.15 (m, 1H), 3.68 (s, 3H), 3.54 (s, 2H), 2.92-2.87 (m, 3H), 2.54 (s, 2H), 2.18-2.13 (m, 2H), 2.07-2.00 (m, 5H), 1.56 (d, J = 8 Hz, 3H). |
| 1108 | 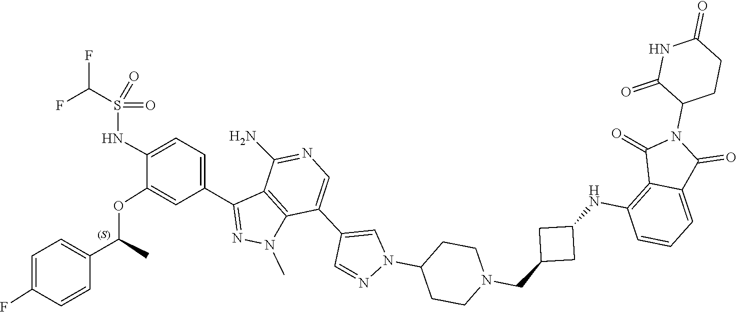 | N-(4-{4-amino-1-methyl-7-[1-(1-{[(trans)-3-{[2- (2,6-dioxopiperidin-3-yl)-1,3-dioxo-2,3-dihydro- 1H-isoindol-4-yl]amino}cyclobutyl]methyl}piperidin- 4-yl)-1H-pyrazol-4-yl]-1H-pyrazolo[4,3-c]pyridin- 3-yl}-2-[(1S)-1-(4-fluorophenyl)ethoxy]phenyl)-1,1- difluoromethane-sulfonamide From Intermediate M6. LCMS (Method B): 3.09 min, m/z: 980.9 [M + H]⁺. ¹H NMR (400 MHZ, DMSO-d₆): 11.11 (s, 1H), 7.99 (s, 1H), 7.60 (s, 2H), 7.57 - 7.45 (m, 3H), 7.38 (d, J = 8.2 Hz, 1H), 7.15 (t, J =8.6 Hz, 2H), 7.10 - 6.60 (m, 5H), 6.48 (dd, J = 21.9, 6.6 Hz, 1H), 5.66 (s, 2H), 5.06 (dd, J = 12.9, 5.5 Hz, 1H), 4.24 (d, J = 14.0 Hz, 1H), 4.02 (q, J = 7.7 Hz, 1H), 3.68 (s, 3H), 3.06 (d, J = 10.5 Hz, 2H), 2.88 (s, 2H), 2.64 (d, J = 19.7 Hz, 5H), 2.35 (d, J = 15.7 Hz, 2H), 2.24 (s, 1H), 2.17 - 1.98 (m, 5H), 1.69 (q, J = 10.3, 9.7 Hz, 2H), 1.55 (d, J = 6.2 Hz, 3H), 1.24 (d, J = 7.4 Hz, 2H). |
| 1136 | 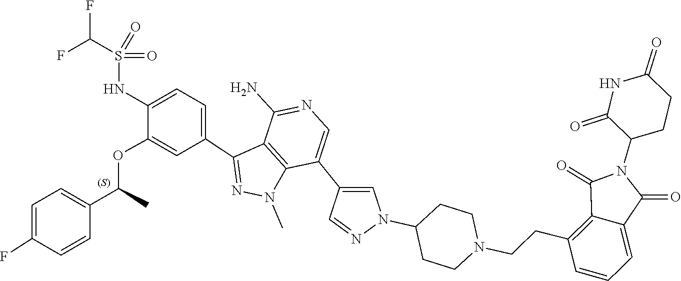 | N-(4-{4-amino-7-[1-(1-{2-[2-(2,6-dioxopiperidin- 3-yl)-1,3-dioxo-2,3-dihydro-1H-isoindol-4- yl]ethyl}piperidin-4-yl)-1H-pyrazol-4-yl]-1-methyl- 1H-pyrazolo[4,3-c]pyridin-3-yl}-2-[(1S)-1-(4-fluorophenyl)ethoxy]phenyl)-1,1- difluoromethanesulfonamide From Intermediate M7. LCMS (Method B): 1.69 min, m/z: 463.3 [M + 2H]²⁺. ¹H NMR (400 MHZ, CDCl₃): 7.78 (s, 1H), 7.68 (s, 3H), 7.63 (s, 1H), 7.57 (s, 2H), 7.53 (s, 1H), 7.32 (s, 3H), 7.19 (s, 1H), 7.04 (s, 3H), 6.35 (t, J = 53.5 Hz, 1H), 5.48 (d, J = 6.6 Hz, 1H), 5.01- 4.94 (m, 1H), 4.34 (s, 1H), 3.78 (s, 3H), 3.42 (s, 2H), 3.18 (s, 2H), 2.95 (d, J = 25.2 Hz, 2H), 2.83 (s, 5H), 2.74 (s, 2H), 2.35 (s, 3H), 2.17 (d, J = 7.8 Hz, 6H), 2.05-1.97 (m, 7H), 1.71 (d, J = 6.4 Hz, 5H), 1.64 (d, J = 6.8 Hz, 2H), 1.50 (s, 2H), 0.88 (t, J = 6.4 Hz, 6H). |
| 1123 | 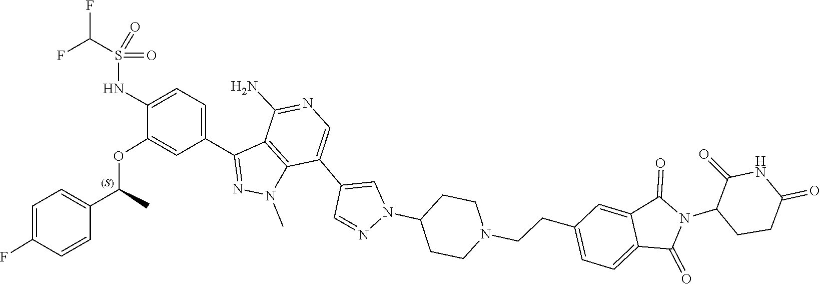 | N-(4-{4-amino-7-[1-(1-{2-[2-(2,6-dioxopiperidin- 3-yl)-1,3-dioxo-2,3-dihydro-1H-isoindol-5- yl]ethyl}piperidin-4-yl)-1H-pyrazol-4-yl]-1- methyl-1H-pyrazolo[4,3-c]pyridin-3-yl}-2-[(1S)- 1-(4-fluorophenyl)ethoxy]phenyl)-1,1- difluoromethanesulfonamide From Intermediate M8. LCMS (Method B): 1.87 min, m/z 463.3 [M + 2H]²⁺. ¹H NMR (400 MHZ, DMSO-d₆): 11.12 (s, 1H), 8.14 (s, 1H), 7.99 (s, 1H), 7.87-7.77 (m, 2H), 7.59 (s, 1H), 7.57-7.48 (m, 3H), 7.38 (d, J = 8.2 Hz, 1H), 7.15 (t, J = 8.6 Hz, 2H), 7.04 (d, J = 11.3 Hz, 2H), 6.74 (t, J = 53.1 Hz, 1H), 5.65 (s, 2H), 5.20-5.10 (m, 1H), 4.22 (s, 1H), 3.68 (s, 3H), 3.09 (d, J = 10.5 Hz, 2H), 3.00 (s, 2H), 2.88 (d, J = 13.2 Hz, 2H), 2.76-2.60 (m, 4H), 2.23 ( s, 2H), 2.03 (d, J = 27.6 Hz, 5H), 1.55 (d, J = 6.1 Hz, 3H). |

### Compound 1115: N-(4-{4-amino-7-[1-(1-{[4-({[2-(2,6-dioxopiperidin-3-yl)-1,3-dioxo-2,3-dihydro-1H-isoindol-4-yl]amino}methyl)phenyl]methyl}piperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

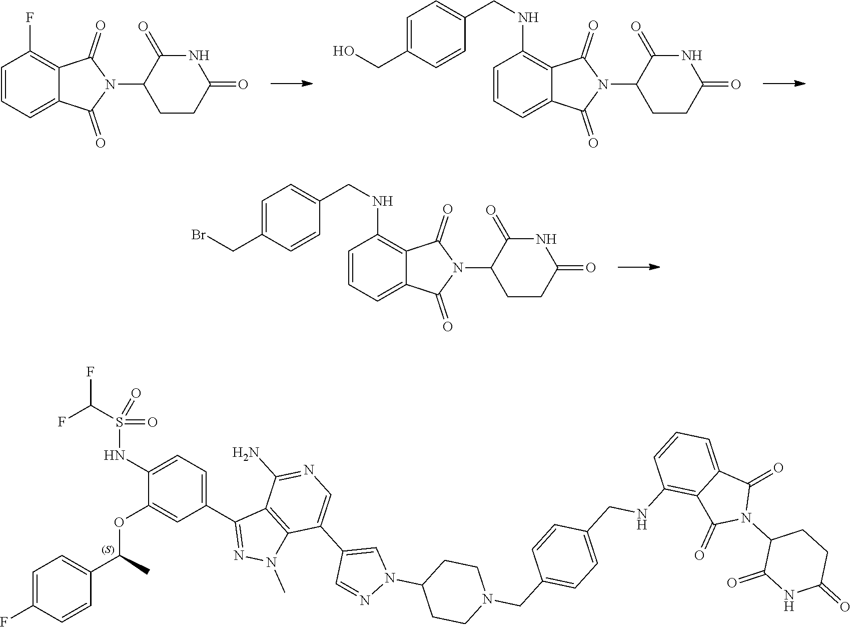

### Step 1: 2-(2,6-dioxopiperidin-3-yl)-4-({[4-(hydroxymethyl)phenyl]methyl}amino)-2,3-dihydro-1H-isoindole-1,3-dione

A mixture of [4-(aminomethyl)phenyl]methanol (300 mg), DIPEA (1 mL) and 2-(2,6-dioxopiperidin-3-yl)-4-fluoro-2,3-dihydro-1H-isoindole-1,3-dione (500 mg) in DMSO (10 mL) was stirred at 70° C. under N₂ for 16 h. The mixture was concentrated and the residue was purified by reverse-phase chromatography to afford the title product (136 mg, 16%) as a yellow-green solid. LCMS (Method A): 1.86 min, m/z: 416.1 [M+Na]⁺.

### Step 2: 4-({[4-(bromomethyl)phenyl]methyl}amino)-2-(2,6-dioxopiperidin-3-yl)-2,3-dihydro-1H-isoindole-1,3-dione

A solution of 2-(2,6-dioxopiperidin-3-yl)-4-({[4-(hydroxymethyl)phenyl]methyl}amino)-2,3-dihydro-1H-isoindole-1,3-dione (40 mg, 102 μmol) in BBr₃ (10 mL) was stirred at RT for 8 h. The mixture was concentrated and the residue was purified by Prep-TLC (DCM:MeOH=20:1) to afford the title product (44.0 mg, 95%) as a yellow-green solid. LCMS (Method A): 2.26 min, m/z: 456.1, 458.1 [M+H]⁺.

### Step 3: N-(4-{4-amino-7-[1-(1-{[4-({[2-(2,6-dioxopiperidin-3-yl)-1,3-dioxo-2,3-dihydro-1H-isoindol-4-yl]amino}methyl)phenyl]methyl}piperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

To a solution of 4-({[4-(bromomethyl)phenyl]methyl}amino)-2-(2,6-dioxopiperidin-3-yl)-2,3-dihydro-1H-isoindole-1,3-dione (44 mg, 96.4 μmol) in MeCN (4 mL) were added N-(4-{4-amino-1-methyl-7-[1-(piperidin-4-yl)-1H-pyrazol-4-yl]-1H-pyrazolo[4,3-c]pyridin-3-yl}-2-[(1 S)-1-(4-fluoro phenyl)ethoxy]phenyl)-1,1-difluoromethanesulfonamide (62 mg, 96.8 μmol) and Et₃N (0.5 mL, 4.94 mmol), and the mixture was stirred at 70° C. under N₂ for 8 h. The mixture was concentrated and the crude residue was purified by Prep-TLC (DCM:MeOH=20:1) to afford the title product (7.0 mg, 7%) as a yellow-green solid. LCMS (Method A): 3.48 min, m/z: 1016.8 [M+H]⁺. ¹H NMR (400 MHz, CDCl₃): 8.49 (s, 1H), 7.67 (dd, J=12.4, 8.0 Hz, 2H), 7.59-7.51 (m, 3H), 7.46 (t, J=7.8 Hz, 2H), 7.41-7.30 (m, 6H), 7.20 (dd, J=8.2, 1.8 Hz, 1H), 7.13 (d, J=7.1 Hz, 1H), 7.09-7.02 (m, 3H), 6.87 (d, J=8.5 Hz, 1H), 6.67 (t, J=5.8 Hz, 1H), 6.35 (t, J=53.6 Hz, 2H), 5.92-5.72 (m, 2H), 5.47 (q, J=6.4 Hz, 1H), 4.98-4.89 (m, 1H), 4.50 (d, J=5.7 Hz, 2H), 4.26 (s, 1H), 3.75 (s, 2H), 3.63 (s, 1H), 3.33-3.24 (m, 1H), 3.09 (d, J=11.4 Hz, 2H), 2.94-2.68 (m, 4H), 2.37-2.21 (m, 8H), 1.70 (d, J=6.3 Hz, 3H).

### Compound 1122: N-(4-{4-amino-7-[1-(1-{[3-({[2-(2,6-dioxopiperidin-3-yl)-1,3-dioxo-2,3-dihydro-1H-isoindol-4-yl]amino}methyl)phenyl]methyl}piperidin-4-yl)-1H-pyrazol-4-yl]-1-methyl-1H-pyrazolo[4,3-c]pyridin-3-yl}-2-fluorophenyl)-1,1-difluoromethanesulfonamide

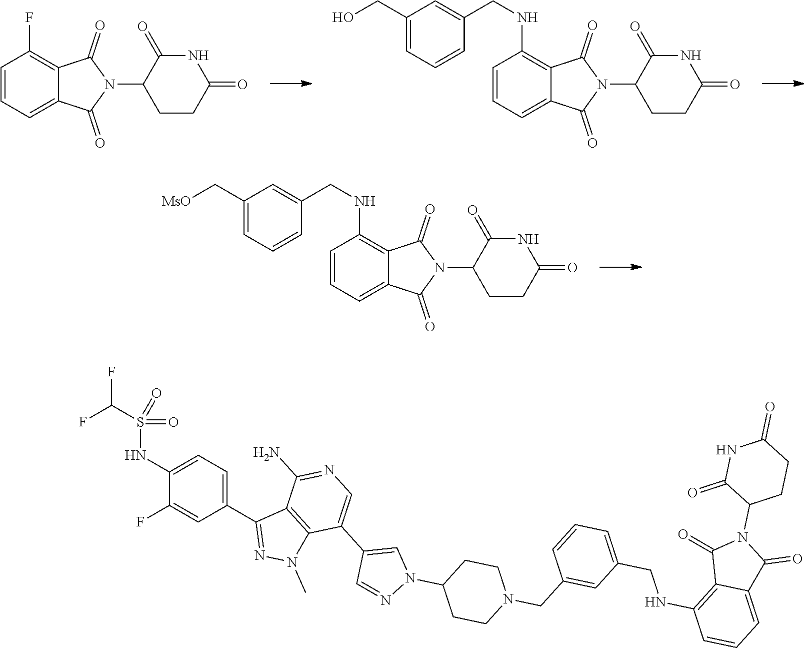 [SMILES: O=C1CCC(N2C(=O)c3cccc(F)c3C2=O)C(=O)N1 ; O=C1CCC(N2C(=O)c3cccc(NCc4cccc(CO)c4)c3C2=O)C(=O)N1 ; CS(=O)(=O)OCc1cccc(CNc2cccc3c2C(=O)N(C2CCC(=O)NC2=O)C3=O)c1 ; Cn1nc(-c2ccc(NS(=O)(=O)C(F)F)c(F)c2)c2c(N)ncc(-c3cnn(C4CCN(Cc5cccc(CNc6cccc7c6C(=O)N(C6CCC(=O)NC6=O)C7=O)c5)CC4)c3)c21]

### Step 1: 2-(2,6-dioxopiperidin-3-yl)-4-({[3-(hydroxymethyl)phenyl]methyl}amino)-2,3-dihydro-1H-isoindole-1,3-dione

A mixture of [3-(aminomethyl)phenyl]methanol (100 mg, 728 μmol), 2-(2,6-dioxopiperidin-3-yl)-4-fluoro-2,3-dihydro-1H-isoindole-1,3-dione (201 mg, 728 μmol) and DIPEA (376 mg, 2.91 mmol) in DMSO (3 mL) was stirred at 70° C. under N₂ overnight. The mixture was concentrated, then the residue was diluted with water and extracted with EtOAc. The combined organic phases were dried over Na₂SO₄ and concentrated. The residue was purified by Prep-TLC (DCM/MeOH=30/1) to give the title product (110 mg, 38%) as a yellow solid. LCMS (Method B): 2.77 min, m/z: 416.1 [M+Na]⁺.

### Step 2: [3-({[2-(2,6-dioxopiperidin-3-yl)-1,3-dioxo-2,3-dihydro-1H-isoindol-4-yl]amino}methyl) phenyl]methyl methanesulfonate

A mixture of 2-(2,6-dioxopiperidin-3-yl)-4-({[3-(hydroxymethyl)phenyl]methyl}amino)-2,3-dihydro-1H-isoindole-1,3-dione (100 mg, 254 μmol), MsCl (58.6 mg, 510 μmol) and Et₃N (103 mg, 1.01 mmol) in DCM (2 mL) was stirred at RT for 2 h. The mixture was concentrated, then the residue was diluted with H₂O (50 mL) and extracted with DCM (3×50 mL). The combine organic phases were dried over Na₂SO₄ and concentrated. The residue was purified by Prep-TLC (DCM:MeOH=20:1) to give the title product (100 mg, 84%) as a yellow solid. LCMS (Method B): 2.02 min, m/z: 494.1 [M+Na]⁺.

### Step 3: N-(4-{4-amino-7-[1-(1-{[3-({[2-(2,6-dioxopiperidin-3-yl)-1,3-dioxo-2,3-dihydro-1H-isoindol-4-yl]amino}methyl)phenyl]methyl}piperidin-4-yl)-1H-pyrazol-4-yl]-1-methyl-1H-pyrazolo[4,3-c]pyridin-3-yl}-2-fluorophenyl)-1,1-difluoromethanesulfonamide

A mixture of [3-({[2-(2,6-dioxopiperidin-3-yl)-1,3-dioxo-2,3-dihydro-1H-isoindol-4-yl]amino}methyl)phenyl]methyl methanesulfonate (52.9 mg, 112 μmol), N-(4-{4-amino-1-methyl-7-[1-(piperidin-4-yl)-1H-pyrazol-4-yl]-1H-pyrazolo[4,3-c]pyridin-3-yl}-2-fluorophenyl)-1,1-difluoro methanesulfonamide (52.9 mg, 135 μmol), and Et₃N (34.1 mg, 337 μmol) in DMF (2 mL) was stirred at 60° C. under N₂ for 16 h. The mixture was concentrated, then the residue was diluted with H₂O (50 mL) and extracted with DCM:MeOH (10:1, 3×50 mL). The combine organic phases were dried over Na₂SO₄ and concentrated. The residue was purified by Prep-TLC (DCM:MeOH=20:1) to give the title product (6.6 mg, 7%) as a yellow solid. LCMS (Method B): 1.48 min, m/z: 448.8 [M+2H]²⁺. ¹H NMR (400 MHz, DMSO-d₆): 11.10 (s, 1H), 8.03 (s, 1H), 7.66 (s, 1H), 7.56 (s, 1H), 7.54-7.48 (m, 2H), 7.46 (d, J=5.1 Hz, 1H), 7.37 (s, 3H), 7.30-7.19 (m, 3H), 7.03 (d, J=7.1 Hz, 1H), 6.95 (d, J=8.6 Hz, 1H), 6.32 (s, 1H), 5.06 (dd, J=12.9, 5.4 Hz, 1H), 4.59 (d, J=6.2 Hz, 2H), 4.36 (s, 1H), 3.90 (s, 2H), 3.72 (s, 3H), 3.16 (s, 2H), 2.89 (s, 1H), 2.61 (s, 1H), 2.56 (d, J=8.5 Hz, 2H), 2.15 (s, 4H), 2.01 (dt, J=15.8, 9.0 Hz, 2H).

### Compound 1103: N-(4-{4-amino-7-[1-(1-{4-[2-(2,6-dioxopiperidin-3-yl)-1,3-dioxo-2,3-dihydro-1H-isoindol-4-yl]piperazine-1-carbonyl}piperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

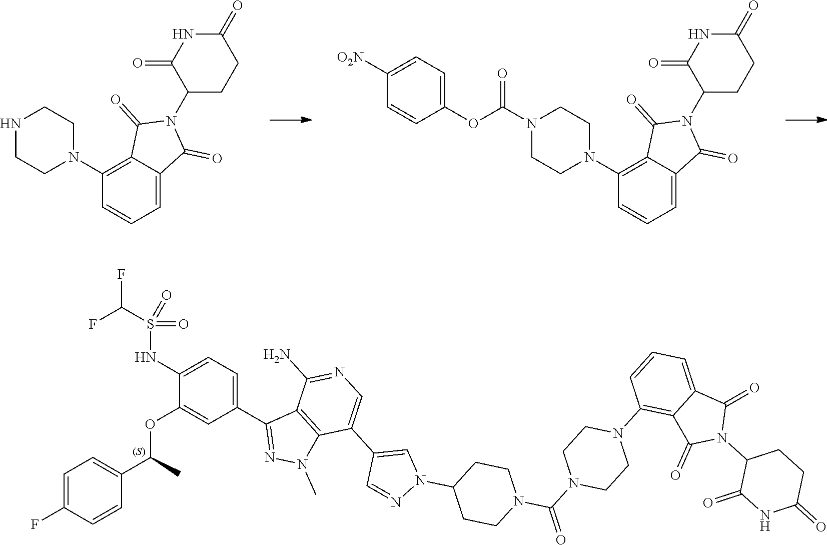

### Step 1: 4-nitrophenyl 4-[2-(2,6-dioxopiperidin-3-yl)-1,3-dioxo-2,3-dihydro-1H-isoindol-4-yl]piperazine-1-carboxylate

A mixture of 2-(2,6-dioxopiperidin-3-yl)-4-(piperazin-1-yl)-2,3-dihydro-1H-isoindole-1,3-dione (50 mg, 146 μmol), 4-nitrophenyl chloroformate (56.2 mg, 279 μmol) and Et₃N (235 mg, 2.33 mmol) in THE (5 mL) was stirred at RT under N₂ for 1 h. The mixture was diluted with H₂O (30 mL) and extracted with EtOAc (3×20 mL). The combined organic layers were dried (Na₂SO₄) and concentrated. The residue was purified by prep-TLC (DCM:MeOH, 25:1) to afford the title compound (38.0 mg, 51%) as a yellow solid. LCMS (Method A2): 3.26 min, m/z: 508.2 [M+H]⁺.

### Step 2: N-(4-{4-amino-7-[1-(1-{4-[2-(2,6-dioxopiperidin-3-yl)-1,3-dioxo-2,3-dihydro-1H-isoindol-4-yl]piperazine-1-carbonyl}piperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

A mixture of 4-nitrophenyl 4-[2-(2,6-dioxopiperidin-3-yl)-1,3-dioxo-2,3-dihydro-1H-isoindol-4-yl]piperazine-1-carboxylate (38 mg, 74.8 μmol), Et₃N (22.6 mg, 224 μmol) and N-(4-{4-amino-1-methyl-7-[1-(piperidin-4-yl)-1H-pyrazol-4-yl]-1H-pyrazolo[4,3-c]pyridin-3-yl}-2-[(1S)-1-(4-fluoro phenyl)ethoxy]phenyl)-1,1-difluoromethanesulfonamide (47.9 mg, 74.8 μmol) in DMF (5 mL) was stirred at 120° C. under N₂ overnight. Once cooled, the mixture was diluted with H₂O (30 mL) and extracted with EtOAc (3×20 mL). The combined organic layers were dried (Na₂SO₄) and then concentrated. The residue was purified by prep-TLC (DCM:MeOH, 25:1) to afford the title compound (19.0 mg, 25%) as a yellow solid. LCMS (Method A2): 2.91 min, m/z 505.2 [M+2H]²⁺. ¹H NMR (400 MHz, DMSO-d₆): 11.08 (s, 1H), 8.13 (s, 1H), 8.01 (s, 1H), 7.73 (t, J=7.8 Hz, 1H), 7.65-7.51 (m, 4H), 7.46-7.32 (m, 3H), 7.20-7.09 (m, 4H), 6.91 (d, J=52.9 Hz, 1H), 5.11 (dd, J=12.8, 5.4 Hz, 1H), 4.43 (tt, J=11.1, 4.2 Hz, 1H), 3.76 (d, J=12.8 Hz, 2H), 3.69 (s, 3H), 3.44-3.37 (m, 8H), 2.98 (t, J=12.4 Hz, 2H), 2.92-2.81 (m, 1H), 2.70-2.54 (m, 2H), 2.28-2.19 (m, 1H), 2.15-2.05 (m, 2H), 1.98 (dq, J=11.7, 5.7, 3.6 Hz, 3H), 1.58 (d, J=6.2 Hz, 3H).

### Compound 1131: N-(4-{4-amino-7-[1-(1-{4-[2-(2,6-dioxopiperidin-3-yl)-1,3-dioxo-2,3-dihydro-1H-isoindol-4-yl]piperazine-1-carbonyl}piperidin-4-yl)-1H-pyrazol-4-yl]-1-methyl-1H-pyrazolo[4,3-c]pyridin-3-yl}-2-fluorophenyl)-1,1-difluoromethanesulfonamide

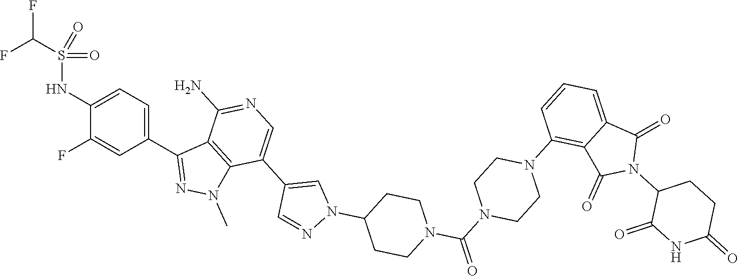 [SMILES: Cn1nc(-c2ccc(NS(=O)(=O)C(F)F)c(F)c2)c2c(N)ncc(-c3cnn(C4CCN(C(=O)N5CCN(c6cccc7c6C(=O)N(C6CCC(=O)NC6=O)C7=O)CC5)CC4)c3)c21]

Compound 1131 was prepared following the procedure for the synthesis of Compound 1103, using N-(4-{4-amino-1-methyl-7-[1-(piperidin-4-yl)-1H-pyrazol-4-yl]-1H-pyrazolo[4,3-c]pyridin-3-yl}-2-fluorophenyl)-1,1-difluoro methanesulfonamide in Step 3. The title compound was obtained as a yellow solid (15 mg, 17%). LCMS (Method B): 1.72 min, m/z: 889.3 [M+H]⁺. ¹H NMR (400 MHz, DMSO-d₆): 11.08 (s, 1H), 8.05 (s, 1H), 7.72 (t, J=7.8 Hz, 1H), 7.64 (s, 1H), 7.56 (s, 1H), 7.48 (t, J=8.6 Hz, 1H), 7.37 (t, J=8.8 Hz, 2H), 7.21 (dd, J=20.3, 10.0 Hz, 2H), 6.46 (t, J=54.2 Hz, 1H), 6.11 (s, 2H), 5.10 (dd, J=13.0, 5.5 Hz, 1H), 4.43 (s, 1H), 3.72 (s, 3H), 3.39 (s, 6H), 3.08 (d, J=7.3 Hz, 4H), 2.98 (t, J=12.4 Hz, 2H), 2.86 (d, J=14.3 Hz, 1H), 2.60 (d, J=16.6 Hz, 2H), 2.10 (d, J=12.2 Hz, 2H), 2.04-1.88 (m, 3H).

### Compound 1105: {1-[2-(2,6-dioxopiperidin-3-yl)-1,3-dioxo-2,3-dihydro-1H-isoindol-4-yl]piperidin-4-yl}methyl4-(4-{4-amino-3-[4-(difluoromethanesulfonamido)-3-[(1 S)-1-(4-fluorophenyl)ethoxy]phenyl]-1-methyl-1H-pyrazolo[4,3-c]pyridin-7-yl}-1H-pyrazol-1-yl)piperidine-1-carboxylate

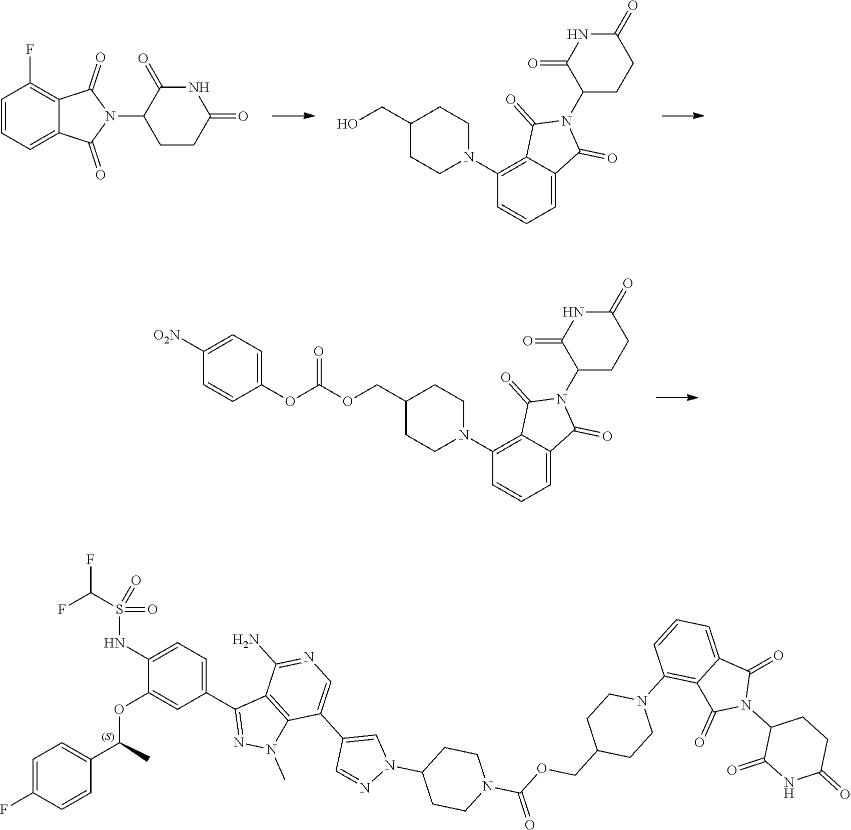

### Step 1: 2-(2,6-dioxopiperidin-3-yl)-4-[4-(hydroxymethyl)piperidin-1-yl]-2,3-dihydro-1H-isoindole-1,3-dione

A mixture of 2-(2,6-dioxopiperidin-3-yl)-4-fluoro-2,3-dihydro-1H-isoindole-1,3-dione (789 mg, 2.86 mmol), (piperidin-4-yl)methanol (300 mg, 2.60 mmol) and DIPEA (135 mg, 10.4 mmol) in DMSO (10 mL) was stirred at 100 ° C. for 4 h. The mixture was concentrated, and the residue was poured into water and extracted with EtOAc. The combine organic phases were dried over Na₂SO₄ and concentrated. The residue was purified by prep-TLC (DCM:MeOH=20:i) to afford the title compound (476.1 mg, 45%) as a yellow solid. LCMS (Method A): 1.44 min m/z: 372.2 [M+H]⁺.

### Step 2: {i-[2-(2,6-dioxopiperidin-3-yl)-1,3-dioxo-2,3-dihydro-1H-isoindol-4-yl]piperidin-4-yl}methyl 4-nitrophenyl carbonate

To a 0° C. solution of 2-(2,6-dioxopiperidin-3-yl)-4-[4-(hydroxymethyl)piperidin-1-yl]-2,3-dihydro-1H-isoindole-1,3-dione (150 mg, 403 μmol) and Et₃N (40.7 mg, 403 μmol) in anhydrous THE (10 mL) was added a solution of 4-nitrophenyl chloroformate (244.38 mg, 1.21 mmol) in DCM (5 mL), dropwise. The reaction was stirred at 0° C. for 12 h, then concentrated and poured into water. The aqueous mixture was extracted with EtOAc, and the combined organics were dried over Na₂SO₄ and concentrated. The residue was purified by prep-TLC (DCM:MeOH=30:i) to afford the title compound (67.5 mg, 68%) as a yellow solid. LCMS (Method A): 3.50 min m/z: 537.2 [M+H]⁺.

### Step 3: {i-[2-(2,6-dioxopiperidin-3-yl)-1,3-dioxo-2,3-dihydro-1H-isoindol-4-yl]piperidin-4-yl}methyl 4-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)piperidine-1-carboxylate

A mixture of {1-[2-(2,6-dioxopiperidin-3-yl)-1,3-dioxo-2,3-dihydro-1H-isoindol-4-yl]piperidin-4-yl}methyl 4-nitrophenyl carbonate (58.1 mg, 108 μmol), N-(4-{4-amino-1-methyl-7-[1-(piperidin-4-yl)-1H-pyrazol-4-yl]-1H-pyrazolo[4,3-c]pyridin-3-yl}-2-[(1 S)-1-(4-fluorophenyl)ethoxy]phenyl)-1,1-difluoromethanesulfonamide (83.25 mg, 129 μmol) and Et₃N (32.90 mg, 325 μmol) in DMF (3 mL) was stirred at RT overnight. The mixture was concentrated, and the residue was poured into water and extracted with EtOAc. The combine organic phases were dried over Na₂SO₄ and concentrated. The residue was purified by prep-TLC (DCM:MeOH=20:1) to afford the title compound (16.5 mg, 15%) as a yellow solid. LCMS (Method A): 1.89 min, m/z: 1039.3 [M+H]⁺. ¹H NMR (400 MHz, DMSO-d₆): 11.08 (s, 1H), 8.02 (s, 1H), 7.68 (dd, J=8.5, 7.1 Hz, 1H), 7.61 (s, 1H), 7.59-7.54 (m, 3H), 7.40 (d, J=8.0 Hz, 1H), 7.33 (t, J=7.9 Hz, 2H), 7.20-7.09 (m, 4H), 7.00 (s, 1H), 5.78 (d, J=21.0 Hz, 2H), 5.65 (q, J=6.3 Hz, 1H), 5.09 (dd, J=12.9, 5.4 Hz, 1H), 4.46 (dq, J=11.4, 7.0, 5.6 Hz, 1H), 4.12 (d, J=13.0 Hz, 2H), 3.97 (d, J=6.1 Hz, 2H), 3.74 (s, 1H), 3.70 (s, 3H), 3.04 (s, 2H), 2.88 (q, J=10.3, 8.4 Hz, 3H), 2.60 (d, J=3.2 Hz, 1H), 2.15-1.98 (m, 3H), 1.95-1.75 (m, 5H), 1.58 (d, J=6.3 Hz, 3H), 1.46 (d, J=12.0 Hz, 2H), 1.24 (d, J=5.7 Hz, 1H).

### Compound 1124: N-[4-(4-amino-7-{1-[1-(3-{[2-(2,6-dioxopiperidin-3-yl)-1,3-dioxo-2,3-dihydro-1H-isoindol-4-yl]amino}azetidine-1-carbonyl)piperidin-4-yl]-1H-pyrazol-4-yl}-1-methyl-1H-pyrazolo[4,3-c]pyridin-3-yl}-2-[(1S)-1-(4-fluorophenyl)ethoxy]phenyl]-1,1-difluoromethanesulfonamide

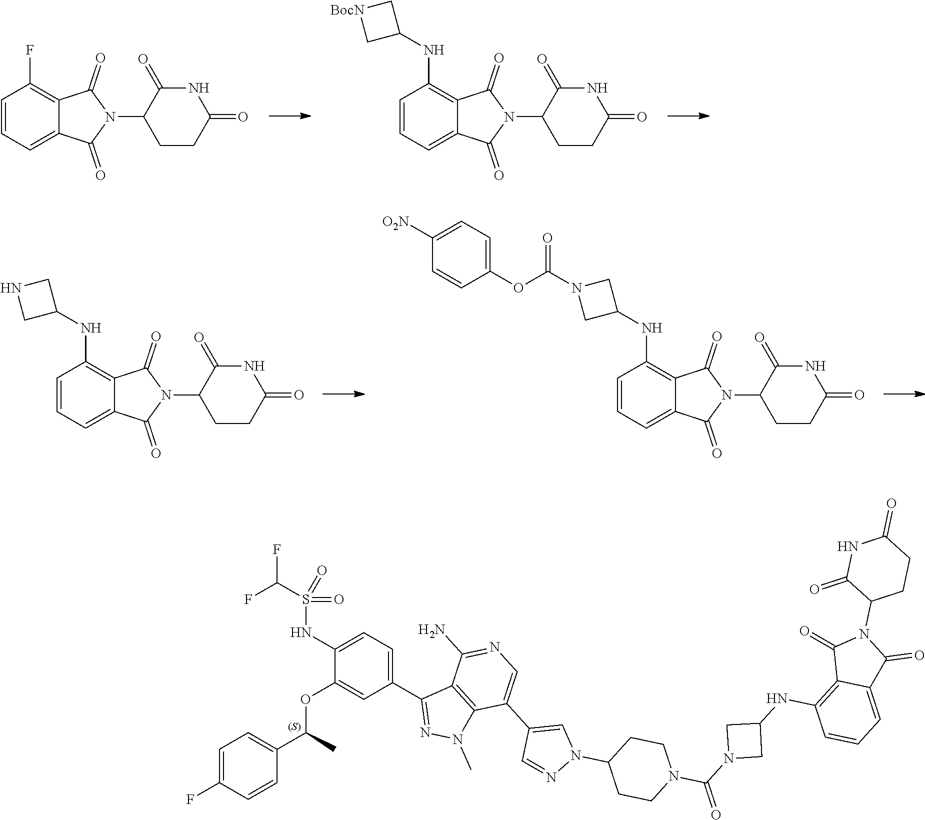

### Step 1: tert-butyl 3-{[2-(2,6-dioxopiperidin-3-yl)-1,3-dioxo-2,3-dihydro-1H-isoindol-4-yl]amino}azetidine-1-carboxylate

A mixture of tert-butyl 3-aminoazetidine-1-carboxylate (172 mg, 998 μmol), DIPEA (515 mg, 3.99 mmol) and 2-(2,6-dioxopiperidin-3-yl)-4-fluoro-2,3-dihydro-1H-isoindole-1,3-dione (328 mg, 1.19 mmol) in DMSO (10 mL) was stirred at 100° C. overnight. The reaction mixture was lyophilized with water (70 mL), then the solid was taken into water (50 mL) and extracted with DCM (3×50 mL). The combined organic phases were dried over Na₂SO₄ and concentrated to give the title product (107 mg, 25%) as a green solid. LCMS (Method B): 3.12 min, m/z: 451.2 [M+Na]⁺.

### Step 2: 4-[(azetidin-3-yl)amino]-2-(2,6-dioxopiperidin-3-yl)-2,3-dihydro-1H-isoindole-1,3-dione

A solution of tert-butyl 3-{[2-(2,6-dioxopiperidin-3-yl)-1,3-dioxo-2,3-dihydro-1H-isoindol-4-yl]amino}azetidine-1-carboxylate (96 mg, 224 μmol) in 4 M HCl/dioxane (10 mL) was stirred at RT for 10 min. The reaction was concentrated to afford the title product (81 mg, 88%) as a yellow solid. LCMS (Method B): 0.89 min, m/z: 329.2 [M+H]⁺.

### Step 3: 4-nitrophenyl 3-{[2-(2,6-dioxopiperidin-3-yl)-1,3-dioxo-2,3-dihydro-1H-isoindol-4-yl]amino}azetidine-1-carboxylate

A mixture of 4-[(azetidin-3-yl)amino]-2-(2,6-dioxopiperidin-3-yl)-2,3-dihydro-1H-isoindole-1,3-dione (76 mg, 231 μmol), 4-nitrophenyl chloroformate (55.8 mg, 277 μmol) and Et₃N (232 mg, 2.30 mmol) in THE (10 mL) was stirred at RT for 1 h. The mixture was concentrated, then diluted with H₂O (40 mL) and extracted with EtOAc (3×50 mL). The combined organics were concentrated, and the residue was purified by Prep-TLC (DCM/MeOH=30:1) to give the title product (40 mg, 35%) as a yellow solid. LCMS (Method B): 2.05 min, m/z: 494.2 [M+H]⁺.

### Step 4: N-[4-(4-amino-7-{1-[1-(3-{[2-(2,6-dioxopiperidin-3-yl)-1,3-dioxo-2,3-dihydro-1H-isoindol-4-yl]amino}azetidine-1-carbonyl)piperidin-4-yl]-1H-pyrazol-4-yl}-1-methyl-1H-pyrazolo[4,3-c]pyridin-3-yl}-2-[(1S)-1-(4-fluorophenyl)ethoxy]phenyl]-1,1-difluoromethanesulfonamide

A mixture of 4-nitrophenyl 3-{[2-(2,6-dioxopiperidin-3-yl)-1,3-dioxo-2,3-dihydro-1H-isoindol-4-yl]amino}azetidine-1-carboxylate (35 mg, 70.9 μmol), N-(4-{4-amino-1-methyl-7-[1-(piperidin-4-yl)-1H-pyrazol-4-yl]-1H-pyrazolo[4,3-c]pyridin-3-yl}-2-[(1 S)-1-(4-fluorophenyl)ethoxy]phenyl)-1,1-difluoromethanesulfonamide (45.4 mg, 70.9 μmol) and Et₃N (21.4 mg, 212 μmol) in DMF (5 mL) was stirred at 120° C. overnight. The mixture was concentrated, then diluted with H2O (40 mL) and extracted with EtOAc (3×50 mL). The combined organics were concentrated, and the residue was purified by Prep-TLC (DCM/MeOH=30:1) to give the title product (3 mg, 4%) as a green solid. LCMS (Method B): 1.93 min, m/z: 498.2 [M+2H]²⁺. ¹H NMR (400 MHz, DMSO-d₆): 11.03 (s, 1H), 7.97 (s, 1H), 7.64 (s, 1H), 7.55 (dd, J=26.0, 8.1 Hz, 5H), 7.37 (d, J=8.0 Hz, 1H), 7.17-7.05 (m, 5H), 6.87 (s, 1H), 6.80 (d, J=8.6 Hz, 1H), 5.72-5.58 (m, 3H), 5.29 (d, J=5.0 Hz, 1H), 5.01 (dd, J=12.9, 5.5 Hz, 1H), 4.44-4.28 (m, 5H), 3.65 (s, 4H), 2.10-1.91 (m, 7H), 1.83 (d, J=11.3 Hz, 2H), 1.55 (d, J=6.3 Hz, 3H), 1.43 (s, 1H), 1.21 (s, 17H), 0.82 (t, J=6.5 Hz, 2H).

### Compound 1133: N-[4-(4-amino-7-{1-[4-(3-{[2-(2,6-dioxopiperidin-3-yl)-1,3-dioxo-2,3-dihydro-1H-isoindol-4-yl]amino}azetidine-1-carbonyl)cyclohexyl]-1H-pyrazol-4-yl}-1-methyl-1H-pyrazolo[4,3-c]pyridin-3-yl}-2-[(1S)-1-(4-fluorophenyl)ethoxy]phenyl]-1,1-difluoromethanesulfonamide

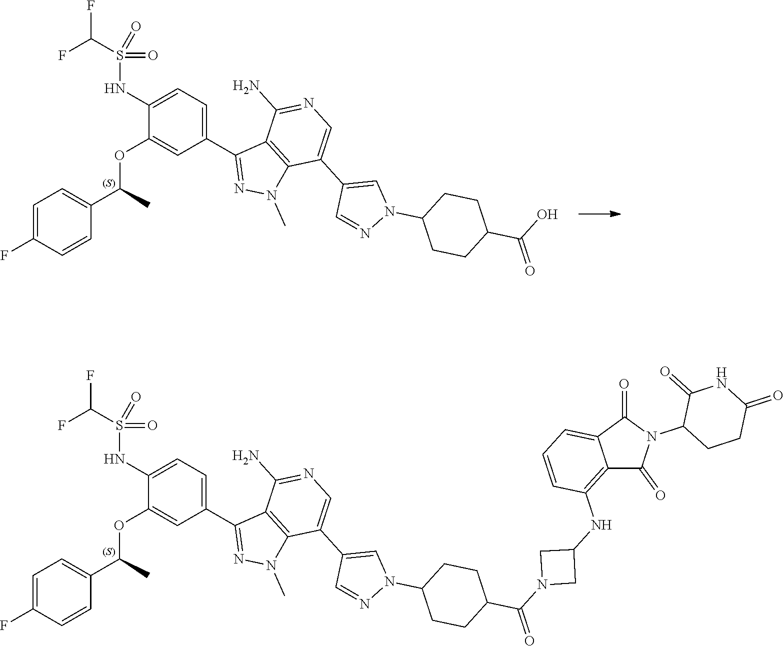

### Step 1: N-[4-(4-amino-7-{1-[4-(3-{[2-(2,6-dioxopiperidin-3-yl)-1,3-dioxo-2,3-dihydro-1H-isoindol-4-yl]amino}azetidine-1-carbonyl)cyclohexyl]-1H-pyrazol-4-yl}-1-methyl-1H-pyrazolo[4,3-c]pyridin-3-yl}-2-[(1S)-1-(4-fluorophenyl)ethoxy]phenyl]-1,1-difluoromethanesulfonamide

A mixture of 4-[(azetidin-3-yl)amino]-2-(2,6-dioxopiperidin-3-yl)-2,3-dihydro-1H-isoindole-1,3-dione (11.1 mg, 34.1 μmol), 4-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)cyclohexane-1-carboxylic acid (18 mg, 26.3 μmol) add NMI (7.55 mg, 92.0 μmol) in MeCN (5 mL) was stirred at RT for 20 min before TCFH (11.0 mg, 39.4 μmol) was added. The reaction was stirred at RT overnight, then concentrated. The residue was diluted with H₂O (50 mL) and extracted with EtOAc (3×50 mL). The combined organics were concentrated, and the residue was purified by Prep-TLC (DCM/MeOH=30/1) and Prep-HPLC to give the title product (10 mg, 17%) as a yellow solid. LCMS (Method B): 1.98 min, m/z: 497.8 [M+2H]²⁺. ¹H NMR (400 MHz, DMSO-d₆): 11.09 (s, 1H), 8.15 (s, 1H), 7.96 (s, 1H), 7.66-7.58 (m, 2H), 7.57-7.51 (m, 3H), 7.38 (d, J=8.1 Hz, 1H), 7.15 (t, J=8.3 Hz, 3H), 7.09-6.96 (m, 3H), 6.93 (d, J=6.2 Hz, 1H), 6.72 (t, J=53.4 Hz, 1H), 5.65 (d, J=6.8 Hz, 2H), 5.07 (dd, J=12.8, 5.4 Hz, 1H), 4.55 (dt, J=15.6, 7.2 Hz, 2H), 4.35-4.20 (m, 2H), 4.17 (d, J=6.1 Hz, 1H), 3.68 (s, 3H), 2.89 (ddd, J=18.6, 14.2, 5.4 Hz, 1H), 2.54 (s, 3H), 2.34 (d, J=9.4 Hz, 2H), 2.03 (dd, J=11.6, 6.0 Hz, 1H), 1.91 (s, 2H), 1.77 (d, J=8.8 Hz, 2H), 1.61 (s, 2H), 1.55 (d, J=6.3 Hz, 3H), 1.24 (s, 1H).

### Compound 1127: N-(4-{4-amino-7-[1-(4-{4-[2-(2,6-dioxopiperidin-3-yl)-1,3-dioxo-2,3-dihydro-1H-isoindol-4-yl}piperidine-1-carbonyl}cyclohexyl)-1H-pyrazol-4-yl]-1-methyl-1H-pyrazolo[4,3-c]pyridin-3-yl-2-[(1S)-1-(4-fluorophenyl)ethoxy]phenyl)-11-difluoromethanesulfonamide

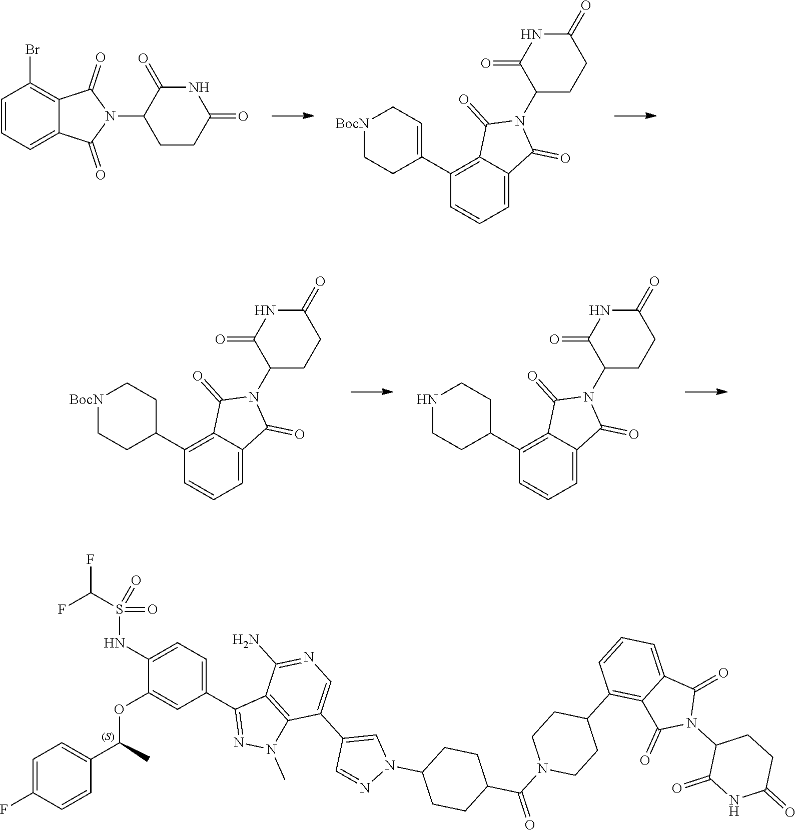

### Step 1: tert-butyl 4-[2-(2,6-dioxopiperidin-3-yl)-1,3-dioxo-2,3-dihydro-1H-isoindol-4-yl]-1,2,3,6-tetrahydropyridine-1-carboxylate

A mixture of 4-bromo-2-(2,6-dioxopiperidin-3-yl)-2,3-dihydro-1H-isoindole-1,3-dione (300 mg, 889 μmol), tert-butyl 4-(4,4,5,5-tetramethyl-1,3,2-dioxaborolan-2-yl)-1,2,3,6-tetrahydropyridine-1-carboxylate (274 mg, 889 μmol), Na₂CO₃ (281 mg, 2.66 mmol) and Pd(dppfCl₂ (64.9 mg, 88.8 μmol) in 1,4-Dioxane (20 mL) was stirred at 100° C. under N₂ for 3 h. The mixture was concentrated, then diluted with water (40 mL) and extracted with EtOAc (40 mL×2). The combined organic phases were concentrated, and the residue was purified by Prep-TLC (DCM/MeOH=30/1) to give the title product (148 mg, 38%) as a yellow solid. LCMS (Method B): 2.36 min, m/z: 462.2 [M+Na]⁺.

### Step 2: tert-butyl 4-[2-(2,6-dioxopiperidin-3-yl)-1,3-dioxo-2,3-dihydro-1H-isoindol-4-yl]piperidine-1-carboxylate

A mixture of tert-butyl 4-[2-(2,6-dioxopiperidin-3-yl)-1,3-dioxo-2,3-dihydro-1H-isoindol-4-yl]-1,2,3,6-tetrahydropyridine-1-carboxylate (143 mg, 325 μmol) and 10% Pd/C (20 mg, 18.7 μmol) in MeOH (12 mL) was stirred at RT under H₂ overnight. The reaction was filtered over Celite, and the filtrate was concentrated to give the title product (143 mg, 100%) as a yellow solid. LCMS (Method B): 2.18 min, m/z: 462.2 [M+Na]⁺.

### Step 3: 2-(2,6-dioxopiperidin-3-yl)-4-(piperidin-4-yl)-2,3-dihydro-1H-isoindole-1,3-dione

A solution of tert-butyl 4-[2-(2,6-dioxopiperidin-3-yl)-1,3-dioxo-2,3-dihydro-1H-isoindol-4-yl]piperidine-1-carboxylate (143 mg, 325 μmol) in 4 M HCl/dioxane (10 mL) was stirred at RT for 20 min. The mixture was concentrated to afford the title product (121 mg, >100%) as a yellow solid. (LCMS (Method B): 1.70, m/z: 342.2 [M+H]⁺.

### Step 4: N-(4-{4-amino-7-[1-(4-{4-[2-(2,6-dioxopiperidin-3-yl)-1,3-dioxo-2,3-dihydro-1H-isoindol-4-yl]piperidine-1-carbonyl}cyclohexyl)-1H-pyrazol-4-yl]-1-methyl-1H-pyrazolo[4,3-c]pyridin-3-yl}-2-[(1 S)-1-(4-fluorophenyl)ethoxy]phenyl)-1,1-difluoromethanesulfonamide

A mixture of 4-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)cyclohexane-1-carboxylic acid (92 mg, 134 μmol), HATU (76.4 mg, 201 μmol) and DIPEA (71.6 mg, 402 μmol) in DMF (5 mL) was stirred at RT for 10 min before 2-(2,6-dioxopiperidin-3-yl)-4-(piperidin-4-yl)-2,3-dihydro-1H-isoindole-1,3-dione (59.7 mg, 175 μmol) was added. The reaction was stirred at RT for 2 h, then concentrated. The mixture was diluted with water (40 mL) and extracted with EtOAc (40 mL×2). The combined organic phases were dried over Na₂SO₄ and concentrated. The residue was purified by prep-TLC (DCM:MeOH=50:1) to afford the title product (3 mg, 2%) as a white solid. LCMS (Method B): 2.14 min, m/z: 1007.3 [M+H]⁺. ¹H NMR (400 MHz, DMSO-d₆): 11.08 (d, J=4.7 Hz, 1H), 8.01 (d, J=3.4 Hz, 1H), 7.84-7.80 (m, 1H), 7.80-7.76 (m, 1H), 7.74 (d, J=5.0 Hz, 1H), 7.60 (s, 1H), 7.57-7.52 (m, 3H), 7.38 (d, J=8.0 Hz, 1H), 7.19-7.06 (m, 5H), 5.62 (d, J=6.4 Hz, 1H), 5.10 (dd, J=12.8, 5.4 Hz, 1H), 4.33 (s, 1H), 4.07 (s, 1H), 3.68 (s, 3H), 2.86 (s, 2H), 2.57 (d, J=14.8 Hz, 2H), 2.38 (s, 2H), 1.97 (dd, J=22.6, 14.9 Hz, 4H), 1.74 (d, J=11.6 Hz, 4H), 1.62 (s, 2H), 1.55 (d, J=6.2 Hz, 3H), 1.43 (d, J=7.6 Hz, 2H), 1.30 (s, 1H), 1.26 (s, 2H).

### Compound 1143: N-(4-{4-amino-7-[1-(4-{4-[2-(2,6-dioxopiperidin-3-yl)-1,3-dioxo-2,3-dihydro-1H-isoindol-5-yl]piperidine-1-carbonyl}cyclohexyl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

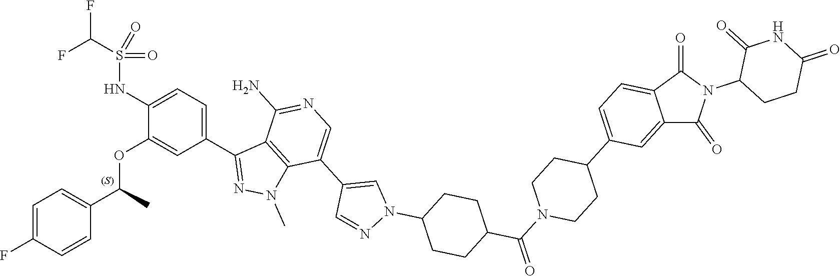

Compound 1143 was prepared from 2-(2,6-dioxopiperidin-3-yl)-5-bromo-2,3-dihydro-1H-isoindole-1,3-dione according to the procedure for the synthesis of Compound 1127. The title compound was obtained as a white solid (12.3 mg, 23%). LCMS (Method B): 1.92 min, m/z: 1007.3 [M+H]⁺. ¹H NMR (400 MHz, DMSO-d₆): 11.11 (s, 1H), 8.04 (s, 1H), 7.85 (d, J=8.9 Hz, 2H), 7.78 (d, J=7.4 Hz, 1H), 7.63 (s, 1H), 7.60-7.55 (m, 3H), 7.41 (d, J=8.0 Hz, 1H), 7.20-7.12 (m, 4H), 7.12-6.87 (m, 2H), 6.09 (s, 2H), 5.66 (d, J=6.4 Hz, 1H), 5.13 (s, 1H), 4.58 (d, J=14.7 Hz, 1H), 4.36 (s, 1H), 4.11 (d, J=13.1 Hz, 1H), 3.71 (s, 3H), 3.16 (s, 1H), 3.09-3.00 (m, 1H), 2.90 (q, J=6.0, 3.4 Hz, 2H), 2.62 (d, J=3.4 Hz, 1H), 2.57 (d, J=4.7 Hz, 1H), 2.41 (s, 2H), 2.10-2.01 (m, 2H), 2.00-1.91 (m, 3H), 1.90-1.72 (m, 5H), 1.64 (s, 2H), 1.58 (d, J=6.3 Hz, 3H), 1.38 (d, J=1.6 Hz, 3H).

### Compound 1144: 4-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)-N-[(1r,3r)-3-{[2-(2,6-dioxopiperidin-3-yl)-1,3-dioxo-2,3-dihydro-1H-isoindol-4-yl]amino}cyclobutyl]cyclohexane-1-carboxamide

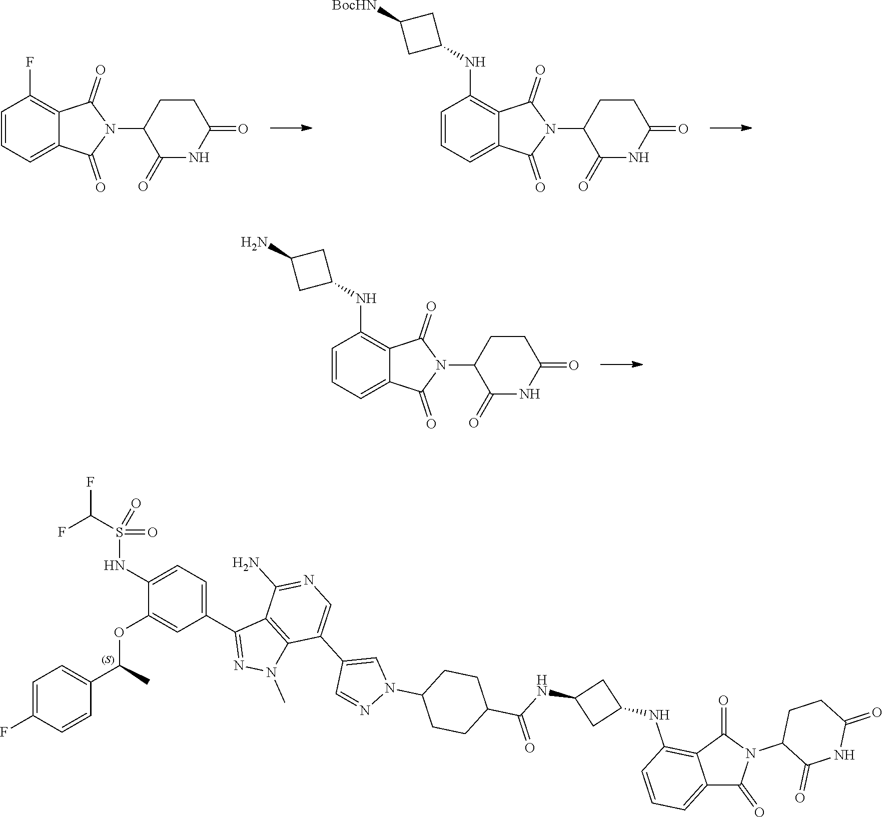

### Step 1: tert-butyl N-[(trans)-3-{[2-(2,6-dioxopiperidin-3-yl)-1,3-dioxo-2,3-dihydro-1H-isoindol-4-yl]amino}cyclobutyl]carbamate

A mixture of 2-(2,6-dioxopiperidin-3-yl)-4-fluoro-2,3-dihydro-1H-isoindole-1,3-dione (397 mg, 1.44 mmol), tert-butyl N-[(trans)-3-aminocyclobutyl]carbamate (400 mg, 2.14 mmol) and DIPEA (744 mg, 5.76 mmol) in DMSO (15 mL) was stirred at 120° C. overnight. Once cooled, the mixture was diluted with H₂O (60 mL) and extracted with EtOAc (40 mL×2). The combined organic phases were concentrated to give the title product (488 mg, 77%) as a yellow solid. LCMS (Method B): 2.24 min, m/z: 465.2 [M+Na]⁺.

### Step 2: 2-(2,6-dioxopiperidin-3-yl)-4-{[(1r,3r)-3-aminocyclobutyl]amino}-2,3-dihydro-1H-isoindole-1,3-dione

A solution of tert-butyl N-[(trans)-3-{[2-(2,6-dioxopiperidin-3-yl)-1,3-dioxo-2,3-dihydro-1H-isoindol-4-yl]amino}cyclobutyl]carbamate (488 mg, 1.10 mmol) in 4 M HCl/dioxane (12 mL) was stirred at RT for 10 min. The mixture was concentrated to afford the title product (394 mg, >100%) as a yellow solid. LCMS (Method B): 1.81 min, m/z: 343.2 [M+H]⁺.

### Step 3: 4-(4-{4-amino-3-[4-(difluoromethanesulfonamido)-3-[(1 S)-1-(4-fluorophenyl)ethoxy]phenyl]-1-methyl-1H-pyrazolo[4,3-c]pyridin-7-yl}-1H-pyrazol-1-yl)-N-[(1r,3r)-3-{[2-(2,6-dioxopiperidin-3-yl)-1,3-dioxo-2,3-dihydro-1H-isoindol-4-yl]amino}cyclobutyl]cyclohexane-1-carboxamide

A mixture of 4-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl-}1H-pyrazol-1-yl)cyclohexane-1-carboxylic acid (25 mg, 36.5 μmol) and 2-(2,6-dioxopiperidin-3-yl)-4-{[(1r,3r)-3-aminocyclobutyl]amino}-2,3-dihydro-1H-isoindole-1,3-dione (16.2 mg, 47.4 μmol) and NMI (10.4 mg, 127 μmol) in MeCN (5 mL) was stirred at RT for 20 min before TCFH (15.3 mg, 54.7 μmol) was added. The reaction was stirred at RT for 2 h, then concentrated. The residue was purified by Prep-HPLC to afford the title product (15 mg, 41%) as a yellow solid. LCMS (Agilent, Method B): 2.16 min; 1008.4 [M+H]⁺. ¹H NMR (400 MHz, DMSO-d₆): 11.09 (s, 1H), 8.18-8.12 (m, 1H), 8.07 (s, 1H), 7.67 (s, 1H), 7.63-7.54 (m, 4H), 7.43 (d, J=8.0 Hz, 1H), 7.22-7.07 (m, 6H), 6.93-6.87 (m, 1H), 6.54 (d, J=5.9 Hz, 1H), 5.65 (d, J=6.4 Hz, 1H), 5.06 (dd, J=12.8, 5.4 Hz, 1H), 4.36-4.25 (m, 2H), 4.24-4.15 (m, 1H), 3.73 (s, 3H), 2.89 (td, J=13.3, 12.6, 6.8 Hz, 1H), 2.64-2.53 (m, 2H), 2.37 (dd, J=11.5, 6.0 Hz, 3H), 2.28 (t, J=7.3 Hz, 3H), 2.08-1.96 (m, 2H), 1.90 (d, J=10.5 Hz, 4H), 1.64 (s, 1H), 1.59 (d, J=6.3 Hz, 3H).

### Compound 1109: 3-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-N-[(trans)-3-{[2-(2,6-dioxopiperidin-3-yl)-1,3-dioxo-2,3-dihydro-1H-isoindol-4-yl]amino}cyclobutyl]prop-2-ynamide

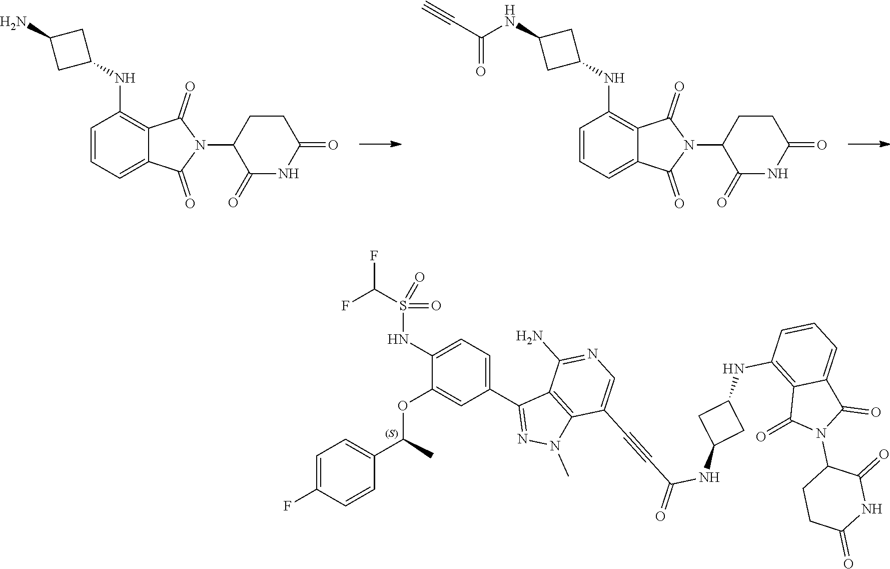

### Step 1: N-[(trans)-3-{[2-(2,6-dioxopiperidin-3-yl)-1,3-dioxo-2,3-dihydro-1H-isoindol-4-yl]amino}cyclobutyl]prop-2-ynamide

A mixture of 2-(2,6-dioxopiperidin-3-yl)-4-{[(trans)-3-aminocyclobutyl]amino}-2,3-dihydro-1H-isoindole-1,3-dione (130 mg, 379 μmol), prop-2-ynoic acid (79.1 mg, 1.13 mmol) DIPEA (244 mg, 1.89 mmol) and HATU (187 mg, 492 μmol) in DMF (3 mL) was stirred at RT for 3 h. The mixture was concentrated, then the residue was diluted with water (40 mL) and extracted with EtOAc (40 mL×2). The combined organic phases were dried over Na₂SO₄ and concentrated. The residue was purified by Prep-TLC (DCM/MeOH=15/1) to give the title product (128 mg, 70%) as a yellow solid. LCMS (Method C): 3.18 min, m/z: 395.5 [M+H]⁺.

### Step 2: 3-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-N-[(trans)-3-{[2-(2,6-dioxopiperidin-3-yl)-1,3-dioxo-2,3-dihydro-1H-isoindol-4-yl]amino}cyclobutyl]prop-2-ynamide

A mixture of N-[(trans)-3-{[2-(2,6-dioxopiperidin-3-yl)-1,3-dioxo-2,3-dihydro-1H-isoindol-4-yl]amino}cyclobutyl]prop-2-ynamide (90 mg, 228 μmol), Cul (4.34 mg, 22.8 μmol), Et₃N (230 mg, 2.28 mmol), N-(4-{4-amino-7-iodo-1-methyl-1H-pyrazolo[4,3-c]pyridin-3-yl}-2-[(1S)-1-(4-fluoro phenyl)ethoxy]phenyl)-1,1-difluoromethanesulfonamide (140 mg, 228 μmol) and Pd(PPh₃)₂Cl₂ (16.0 mg, 22.8 μmol) in DMSO (3 mL) was stirred in a sealed tube at 70° C. under N₂ (50 psi) for 2 h. The mixture was concentrated, then the residue was diluted with water (40 mL) and extracted with EtOAc (40 mL×2). The combined organic phases were dried over Na₂SO₄ and concentrated. The residue was purified by Prep-TLC (DCM/MeOH=15/1) to give the title product (22 mg, 11%) as a yellow solid. LCMS (Method B): 1.95 min, m/z: 884.3 [M+H]⁺. ¹H NMR (400 MHz, DMSO-d₆): 11.11 (s, 1H), 10.50 (s, 1H), 9.07 (d, J=7.0 Hz, 1H), 8.06 (s, 1H), 7.65-7.61 (m, 1H), 7.60-7.55 (m, 2H), 7.41 (d, J=8.5 Hz, 1H), 7.19-7.08 (m, 5H), 6.93 (d, J=8.5 Hz, 1H), 6.60 (d, J=6.0 Hz, 1H), 6.39 (s, 1H), 5.67 (q, J=6.3 Hz, 1H), 5.07 (dd, J=12.8, 5.4 Hz, 1H), 4.38 (q, J=7.2 Hz, 1H), 4.24 (s, 3H), 2.90 (ddd, J=17.3, 14.0, 5.5 Hz, 1H), 2.60 (d, J=17.4 Hz, 1H), 2.54 (s, 3H), 2.41-2.31 (m, 3H), 2.04 (dt, J=7.6, 2.9 Hz, 1H), 1.58 (d, J=6.2 Hz, 3H).

### Compound 1145: 5-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)-1-methyl-N-[(trans)-3-{[2-(2,6-dioxopiperidin-3-yl)-1,3-dioxo-2,3-dihydro-1H-isoindol-4-yl]amino}cyclobutyl]piperidine-2-carboxamide

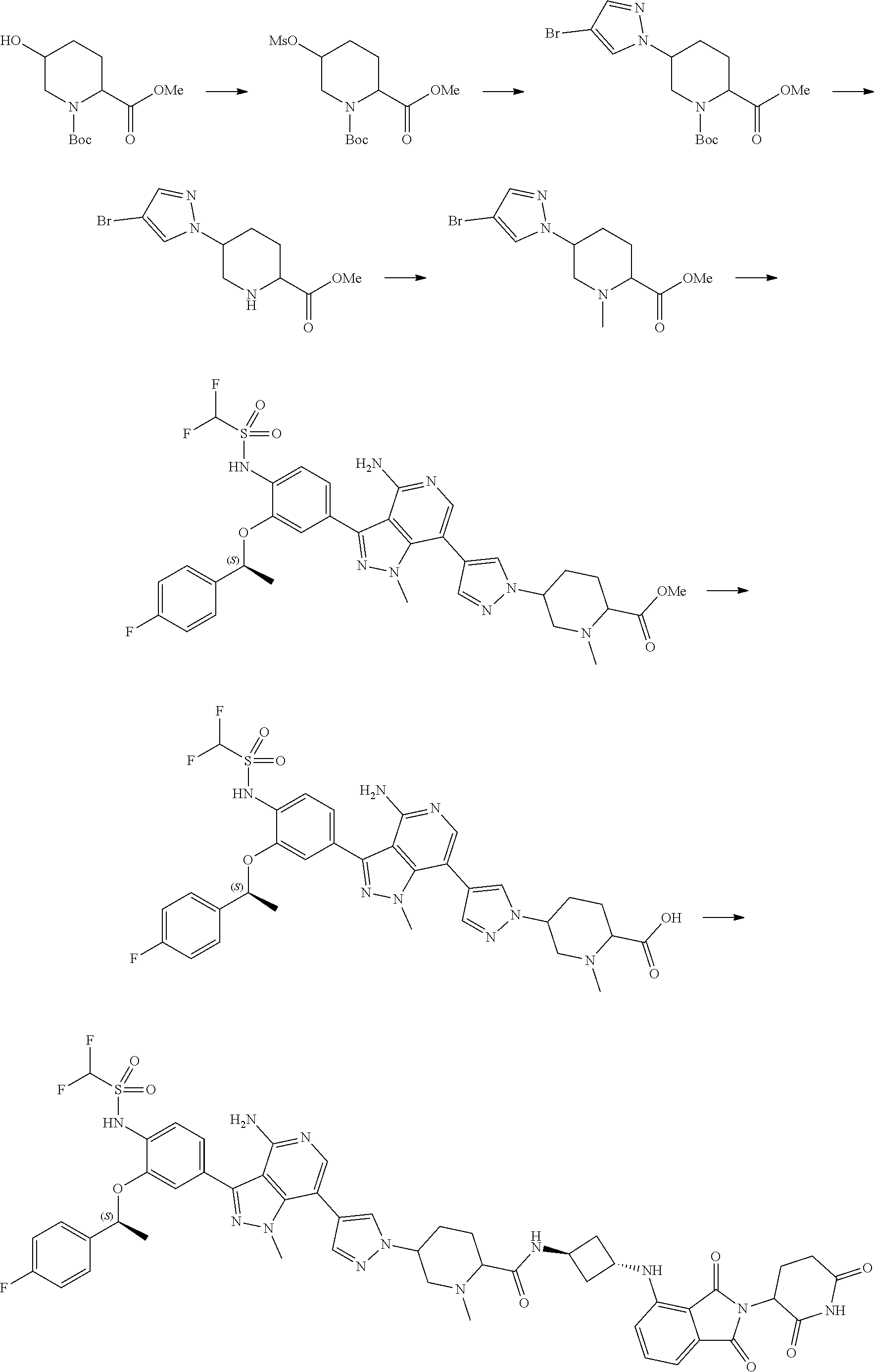

### Step 1: 1-tert-butyl 2-methyl 5-(methanesulfonyloxy)piperidine-1,2-dicarboxylate

To a mixture of 1-tert-butyl 2-methyl 5-hydroxypiperidine-1,2-dicarboxylate (1 g, 3.85 mmol) and Et₃N (1.16 g, 11.5 mmol) in DCM (20 mL) was added MsCl (572 mg, 5.00 mmol), and the reaction was stirred at RT for 2 h. The mixture was washed with water (30 mL), dried over Na₂SO₄ and concentrated to give the title product (1.30 g, 100%) as a yellow oil. LCMS (Method B): 2.21 min, 360.1 [M+Na]⁺.

### Step 2: 1-tert-butyl 2-methyl 5-(4-bromo-1H-pyrazol-1-yl)piperidine-1,2-dicarboxylate

A mixture of 1-tert-butyl 2-methyl 5-(methanesulfonyloxy)piperidine-1,2-dicarboxylate (1.3 g, 3.85 mmol), 4-bromo-1H-pyrazole (848 mg, 5.77 mmol) and Cs₂CO₃ (3.74 g, 11.5 mmol) in MeCN (20 mL) was stirred at 80° C. for 4 h. The mixture was concentrated, and the residue was purified by column chromatography (PE/EtOAc=10/1) to give the title product (600 mg, 40%) as a colourless oil. LCMS (Method B): 2.63 min, 410.1, 412.1 [M+Na]⁺.

### Step 3: methyl 5-(4-bromo-1H-pyrazol-1-yl)piperidine-2-carboxylate

A solution of 1-tert-butyl 2-methyl 5-(4-bromo-1H-pyrazol-1-yl)piperidine-1,2-dicarboxylate (700 mg, 1.80 mmol) in 4 M HCl/dioxane (3 mL) was stirred at RT for 1 h. The mixture was concentrated to give the title product (600 mg, >100%) as a white solid. LCMS (Method B): 0.93 min, m/z: 288.0, 290.0 [M+H]⁺.

### Step 4: methyl 5-(4-bromo-1H-pyrazol-1-yl)-1-methylpiperidine-2-carboxylate

To a mixture of methyl 5-(4-bromo-1H-pyrazol-1-yl)piperidine-2-carboxylate (0.6 g, 2.08 mmol) and CH₂O (314 mg, 10.4 mmol) in DCM (20 mL) was added NaBH(OAc)₃ (881 mg, 4.16 mmol), and the reaction was stirred at RT for 1 h. The mixture was washed with H₂O (20 mL), dried over Na₂SO₄ and concentrated to give the title product (420 mg, 67%) as a white solid. LCMS (Method B): 1.22 min, 302.1, 304.1 [M+H]⁺.

### Step 5: methyl 5-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)-1-methylpiperidine-2-carboxylate

A mixture of methyl 5-(4-bromo-1H-pyrazol-1-yl)-1-methylpiperidine-2-carboxylate (420 mg, 1.38 mmol), N-(4-{4-amino-7-iodo-1-methyl-1H-pyrazolo[4,3-c]pyridin-3-yl}-2-[(1S)-1-(4-fluorophenyl) ethoxy]phenyl)-1,1-difluoromethanesulfonamide (851 mg, 1.38 mmol), bis(adamantan-1-yl)(butyl)phosphane (494 mg, 1.38 mmol), K₂CO₃ (191 mg, 1.38 mmol) B₂(pin)₂ (700 mg, 2.76 mmol) and Pd(OAc)₂ (156 mg, 690 μmol) in 1,4-dioxane/H₂O (4/1, 100 mL) was stirred at 80° C. under N₂ overnight. The mixture was concentrated, and the residue was purified by column chromatography (DCM/MeOH=20/1) to give the title product (500 mg, 51%) as a yellow solid. LCMS (Method B): 1.70 min, m/z: 713.3 [M+H]⁺.

### Step 6: 5-(4-{4-amino-3-[4-(difluoromethanesulfonamido)-3-[(1 S)-1-(4-fluorophenyl)ethoxy]phenyl]-1-methyl-1H-pyrazolo[4,3-c]pyridin-7-yl}-1H-pyrazol-1-yl)-1-methylpiperidine-2-carboxylic acid

A solution of methyl 5-(4-{4-amino-3-[4-(difluoromethanesulfonamido)-3-[(1S)-1-(4-fluorophenyl) ethoxy]phenyl]-1-methyl-1H-pyrazolo[4,3-c]pyridin-7-yl}-1H-pyrazol-1-yl)-1-methylpiperidine-2-carboxylate (0.5 g, 701 μmol) in MeOH (10 mL) and 2 M NaOH (10 mL) was stirred at 60° C. for 1 h. The mixture was acidified to pH=3 with 1 M HCl, then concentrated and extracted with DCM. The combined organics were dried over Na₂SO₄ and concentrated to give the title product (400 mg, 82%) as a yellow solid. LCMS (Method B): 1.64 min, m/z: 700.3 [M+H]⁺.

### Step 7: 5-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)-1-methyl-N-[(trans)-3-{[2-(2,6-dioxopiperidin-3-yl)-1,3-dioxo-2,3-dihydro-1H-isoindol-4-yl]amino}cyclobutyl]piperidine-2-carboxamide

A mixture of 5-(4-{4-amino-3-[4-(difluoromethanesulfonamido)-3-[(1S)-1-(4-fluorophenyl)ethoxy]phenyl]-1-methyl-1H-pyrazolo[4,3-c]pyridin-7-yl}-1H-pyrazol-1-yl)-1-methylpiperidine-2-carboxylic acid (100 mg, 143 μmol), 2-(2,6-dioxopiperidin-3-yl)-4-{[(trans)-3-aminocyclobutyl]amino}-2,3-dihydro-1H-isoindole-1,3-dione (48.9 mg, 143 μmol) HATU (65.0 mg, 171 μmol) and DIPEA (55.4 mg, 429 μmol) in DMF (3 mL) was stirred at RT for 3 h. The mixture was concentrated, then the residue was diluted with water (40 mL) and extracted with EtOAc (40 mL×2). The combined organic phases were dried over Na₂SO₄ and concentrated. The residue was purified by Prep-HPLC to give the title product (8 mg, 5%) as a yellow solid. LCMS (Method B): 1.64 min, m/z: 512.3 [M+2H]²⁺. ¹H NMR (400 MHz, DMSO-d₆): 11.11 (s, 1H), 10.50 (s, 1H), 9.07 (d, J=7.0 Hz, 1H), 8.06 (s, 1H), 7.65-7.61 (m, 1H), 7.60-7.55 (m, 2H), 7.41 (d, J=8.5 Hz, 1H), 7.19-7.08 (m, 5H), 6.93 (d, J=8.5 Hz, 1H), 6.60 (d, J=6.0 Hz, 1H), 6.39 (s, 1H), 5.67 (q, J=6.3 Hz, 1H), 5.07 (dd, J=12.8, 5.4 Hz, 1H), 4.38 (q, J=7.2 Hz, 1H), 4.24 (s, 3H), 2.90 (ddd, J=17.3, 14.0, 5.5 Hz, 1H), 2.60 (d, J=17.4 Hz, 1H), 2.54 (s, 3H), 2.41-2.31 (m, 3H), 2.04 (dt, J=7.6, 2.9 Hz, 1H), 1.58 (d, J=6.2 Hz, 3H).

### Compound 1121: N-(4-{4-amino-7-[1-(6-{4-[2-(2,6-dioxopiperidin-3-yl)-1,3-dioxo-2,3-dihydro-1H-isoindol-4-yl]piperazine-1-carbonyl}-1-methylpiperidin-3-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

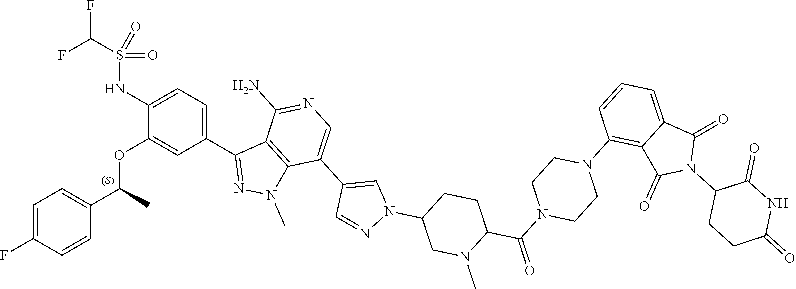

Compound 1121 was prepared according to the procedure for the synthesis of Compound 1145, using 2-(2,6-dioxopiperidin-3-yl)-4-(piperazin-1-yl)-2,3-dihydro-1H-isoindole-1,3-dione in Step 7. The title compound was obtained as a yellow solid (70 mg, 24%). 1 HNMR (400 MHz, DMSO-d₆): 11.11 (s, 1H), 8.03 (s, 1H), 7.84 (d, J=8.4 Hz, 1H), 7.63 (s, 1H), 7.58-7.54 (m, 3H), 7.46 (d, J=2.4 Hz, 1H), 7.41-7.35 (m, 2H), 7.18-7.09 (m, 4H), 6.97 (t, J=52.8 Hz, 1H), 5.90 (s, 2H), 5.65 (q, J=6.4 Hz, 1H), 5.19 (d, J=4.8 Hz, 2H), 5.11 (dd, J=5.2 Hz, 12.8 Hz, 1H), 4.56-4.40 (m, 2H), 3.95 (d, J=13.6 Hz, 1H), 3.70 (s, 3H), 3.27-3.22 (m, 1H), 2.90-2.84 (m, 2H), 2.67-2.55 (m, 2H), 2.14-2.03 (m, 4H), 1.91-1.84 (m, 1H), 1.57 (d, J=6.4 Hz, 3H).

### Intermediate N1: 2-[2-({[2-(2,6-dioxopiperidin-3-yl)-1,3-dioxo-2,3-dihydro-1H-isoindol-4-yl]oxy}methyl)pyrrolidin-1-yl]ethyl methanesulfonate

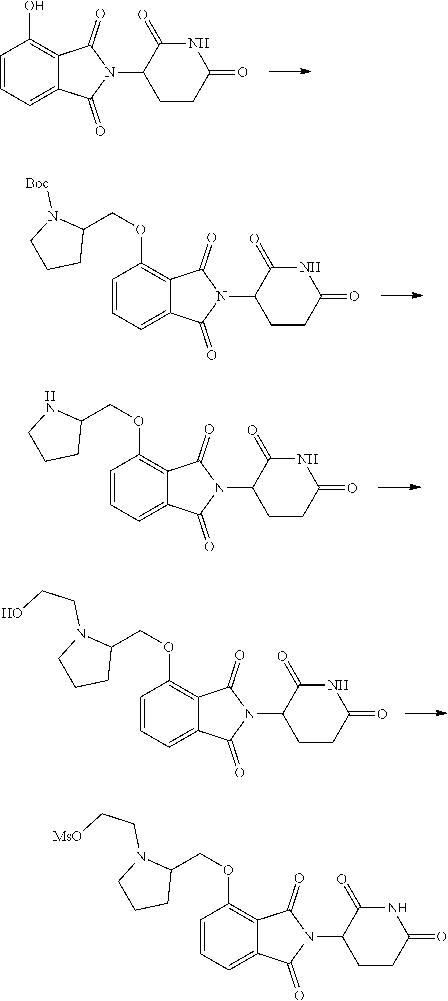 [SMILES: O=C1CCC(N2C(=O)c3cccc(O)c3C2=O)C(=O)N1 ; CC(C)(C)OC(=O)N1CCCC1COc1cccc2c1C(=O)N(C1CCC(=O)NC1=O)C2=O ; O=C1CCC(N2C(=O)c3cccc(OCC4CCCN4)c3C2=O)C(=O)N1 ; O=C1CCC(N2C(=O)c3cccc(OCC4CCCN4CCO)c3C2=O)C(=O)N1 ; CS(=O)(=O)OCCN1CCCC1COc1cccc2c1C(=O)N(C1CCC(=O)NC1=O)C2=O]

### Step 1: tert-butyl 2-({[2-(2,6-dioxopiperidin-3-yl)-1,3-dioxo-2,3-dihydro-1H-isoindol-4-yl]oxy}methyl)pyrrolidine-1-carboxylate

A mixture of 2-(2,6-dioxopiperidin-3-yl)-4-hydroxy-2,3-dihydro-1H-isoindole-1,3-dione (500 mg, 1.82 mmol), tert-butyl 2-(hydroxymethyl)pyrrolidine-1-carboxylate (440.36 mg, 2.18 mmol), PPh₃ (478.53 mg, 1.82 mmol) and DIAD (368.92 mg, 1.81 mmol) in THE (10 mL) was stirred at RT for 12 h. The mixture was concentrated, then the residue was diluted with H₂O (50 mL) and extracted with EtOAc (3×50 mL). The combine organic phases were dried over Na₂SO₄ and concentrated. The residue was purified by column chromatography (PE:EtOAc=2:1) to give the title product (967.2 mg, >100%) as a yellow solid. LCMS (Method B): 2.10 min, m/z: 480.2 [M+Na]⁺.

### Step 2: 2-(2,6-dioxopiperidin-3-yl)-4-[(pyrrolidin-2-yl)methoxy]-2,3-dihydro-1H-isoindole-1,3-dione

A solution of tert-butyl 2-({[2-(2,6-dioxopiperidin-3-yl)-1,3-dioxo-2,3-dihydro-1H-isoindol-4-yl]oxy}methyl)pyrrolidine-1-carboxylate (967.2 mg, 2.11 mmol) in 4 M HCl/dioxane (2 mL) was stirred at RT for 2 h. The mixture was concentrated to give the title product (699.3 mg, 93%) as a yellow solid. LCMS (Method B): 0.84 min, m/z: 358.2 [M+H]⁺.

### Step 3: 2-(2,6-dioxopiperidin-3-yl)-4-{[1-(2-hydroxyethyl)pyrrolidin-2-yl]methoxy}-2,3-dihydro-1H-isoindole-1,3-dione

A mixture of 2-(2,6-dioxopiperidin-3-yl)-4-[(pyrrolidin-2-yl)methoxy]-2,3-dihydro-1H-isoindole-1,3-dione (699.3 mg, 1.95 mmol), 2-bromoethan-1-ol (489.37 mg, 3.91 mmol) and Et₃N (197 mg, 1.95 mmol) in MeCN (10 mL) was stirred at 60° C. for 3 h. The mixture was concentrated, then the residue was diluted with H₂O (50 mL) and extracted with EtOAc (3×50 mL). The combine organic phases were dried over Na₂SO₄ and concentrated. The residue was purified by Prep-TLC (DCM:MeOH=10:1) to give the title product (308.5 mg, 39%) as a yellow solid. LCMS (Method B): 0.72 min, m/z: 402.2 [M+H]⁺.

### Step 4: 2-[2-({[2-(2,6-dioxopiperidin-3-yl)-1,3-dioxo-2,3-dihydro-1H-isoindol-4-yl]oxy}methyl) pyrrolidin-1-yl]ethyl methanesulfonate

A mixture of 2-(2,6-dioxopiperidin-3-yl)-4-{[1-(2-hydroxyethyl)pyrrolidin-2-yl]methoxy}-2,3-dihydro-1H-isoindole-1,3-dione (200 mg, 498 μmol), MsCl (171.3 mg, 1.49 mmol) and Et₃N (504.5 mg, 4.98 mmol) in DCM (4 mL) was stirred at RT for 2 h. The mixture was concentrated, then the residue was diluted with H₂O (50 mL) and extracted with EtOAc (3×50 mL). The combine organic phases were dried over Na₂SO₄ and concentrated to give the title product (124.6 mg, 52%) as a yellow solid. LCMS (Method B): 0.74 min, m/z: 480.2 [M+H]⁺.

### Intermediate N2: 2-[3-({[2-(2,6-dioxopiperidin-3-yl)-1,3-dioxo-2,3-dihydro-1H-isoindol-4-yl]oxy}methyl)piperidin-1-yl]ethyl methanesulfonate

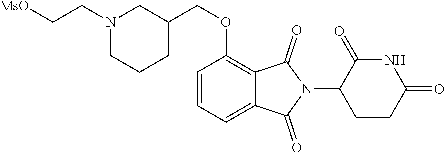 [SMILES: CS(=O)(=O)OCCN1CCCC(COc2cccc3c2C(=O)N(C2CCC(=O)NC2=O)C3=O)C1]

Intermediate N2 was prepared according to the procedure for the synthesis of Intermediate N1 using tert-butyl 3-(hydroxymethyl)piperidine-1-carboxylate in Step 1. The title compound was obtained as a yellow solid (112 mg, 47%). LCMS (Method B): 1.64 min, m/z: 494.1 [M+H]⁺.

### Compound 1120: N-(4-{4-amino-7-[1-(1-{2-[2-({[2-(2,6-dioxopiperidin-3-yl)-1,3-dioxo-2,3-dihydro-1H-isoindol-4-yl]oxy}methyl)pyrrolidin-1-yl]ethyl}piperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

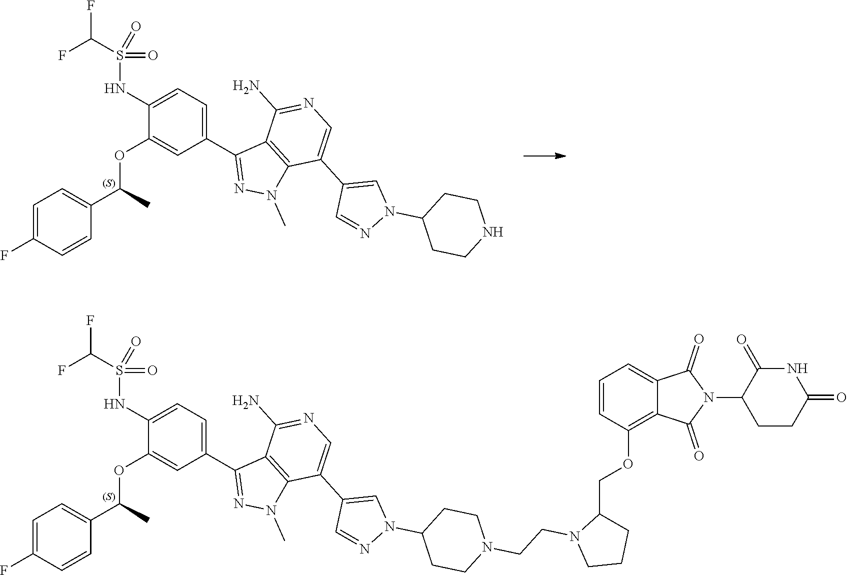

### Step 1: N-(4-{4-amino-7-[1-(1-{2-[2-({[2-(2,6-dioxopiperidin-3-yl)-1,3-dioxo-2,3-dihydro-1H-isoindol-4-yl]oxy}methyl)pyrrolidin-1-yl]ethyl}piperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

A mixture of 2-[2-({[2-(2,6-dioxopiperidin-3-yl)-1,3-dioxo-2,3-dihydro-1H-isoindol-4-yl]oxy}methyl) pyrrolidin-1-yl]ethyl methanesulfonate (124.6 mg, 258 μmol), N-(4-{4-amino-1-methyl-7-[1-(piperidin-4-yl)-1H-pyrazol-4-yl]-1H-pyrazolo[4,3-c]pyridin-3-yl}-2-[(1S)-1-(4-fluorophenyl)ethoxy]phenyl)-1,1-difluoromethanesulfonamide (148.4 mg, 231 μmol) and Et₃N (78.94 mg, 779 μmol) in DMF (4 mL) was stirred at 60° C. under N₂ for 16 h. The mixture was concentrated, then the residue was diluted with H₂O (50 mL) and extracted with EtOAc (3×50 mL). The combine organic phases were dried over Na₂SO₄ and concentrated. The residue was purified by Prep-TLC (DCM:MeOH=10:1) to give the title product (5.9 mg, 2%) as a yellow solid. LCMS (Method B): 1.61 min, m/z: 512.8 [M+2H]²⁺. ¹H NMR (400 MHz, DMSO-d₆): 11.10 (s, 1H), 7.94 (d, J=2.2 Hz, 1H), 7.83 (t, J=7.9 Hz, 1H), 7.58 (d, J=1.7 Hz, 1H), 7.56 (d, J=3.0 Hz, 1H), 7.55-7.49 (m, 3H), 7.46 (d, J=7.3 Hz, 1H), 7.38 (d, J=8.1 Hz, 1H), 7.15 (t, J=8.8 Hz, 2H), 7.03 (dd, J=8.1, 1.9 Hz, 1H), 6.99 (d, J=1.8 Hz, 1H), 5.65 (d, J=5.6 Hz, 2H), 5.13-5.04 (m, 1H), 4.22 (d, J=5.6 Hz, 2H), 3.66 (s, 3H), 3.25 (s, 2H), 3.18 (d, J=6.3 Hz, 2H), 2.89-2.81 (m, 1H), 2.72 (d, J=11.6 Hz, 2H), 2.61-2.52 (m, 2H), 2.37 (d, J=10.8 Hz, 1H), 2.07-1.98 (m, 5H), 1.82 (d, J=7.8 Hz, 1H), 1.74-1.66 (m, 1H), 1.56 (s, 2H).

### Compound 1132: N-(4-{4-amino-7-[1-(1-{2-[3-({[2-(2,6-dioxopiperidin-3-yl)-1,3-dioxo-2,3-dihydro-1H-isoindol-4-yl]oxy}methyl)piperidin-1-yl]ethyl}piperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

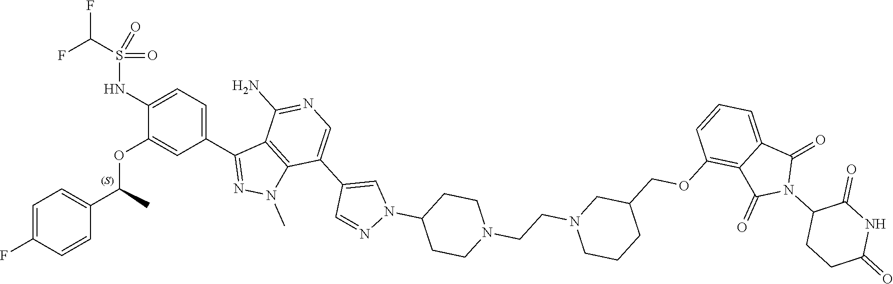

Compound 1132 was prepared according to the procedure for the synthesis of Compound 1120 using Intermediate N2 in Step 1. The title compound was obtained as a yellow solid (23 mg, 11%). LCMS (Method B): 1.65 min, m/z: 520.2 [M+2H]²⁺. ¹H NMR (400 MHz, DMSO-d₆): 9.31 (s, 1H), 8.03 (s, 1H), 7.88-7.74 (m, 1H), 7.59-7.50 (m, 3H), 7.38 (d, J=8.1 Hz, 1H), 7.20-7.10 (m, 2H), 7.08-6.98 (m, 2H), 6.91-6.58 (m, 1H), 5.67 (d, J=5.1 Hz, 2H), 5.04 (d, J=13.1 Hz, 1H), 4.66 (tt, J=11.1, 4.0 Hz, 1H), 4.11 (d, J=13.4 Hz, 1H), 3.69 (s, 3H), 3.20 (dd, J=12.8, 3.0 Hz, 3H), 3.03 (qd, J=7.3, 5.5 Hz, 2H), 2.54 (s, 1H), 2.24 (dd, J=13.1, 4.2 Hz, 2H), 2.12 (s, 1H), 2.08-1.85 (m, 3H), 1.77 (s, 2H), 1.55 (d, J=6.3 Hz, 3H), 1.14 (s, 1H), 0.99 (t, J=7.2 Hz, 2H).

### Compound 1128: N-(4-{4-amino-7-[1-(1-{2-[4-(2-{[2-(2,6-dioxopiperidin-3-yl)-1,3-dioxo-2,3-dihydro-1H-isoindol-4-yl]amino}ethyl)-1H-1,2,3-triazol-1-yl]ethyl}piperidin-4-yl)-1H-pyrazol-4-yl]-1-methyl-1H-pyrazolo[4,3-c]pyridin-3-yl}-2-[(1 S)-1-(4-fluorophenyl)ethoxy]phenyl)-1,1-difluoromethanesulfonamide

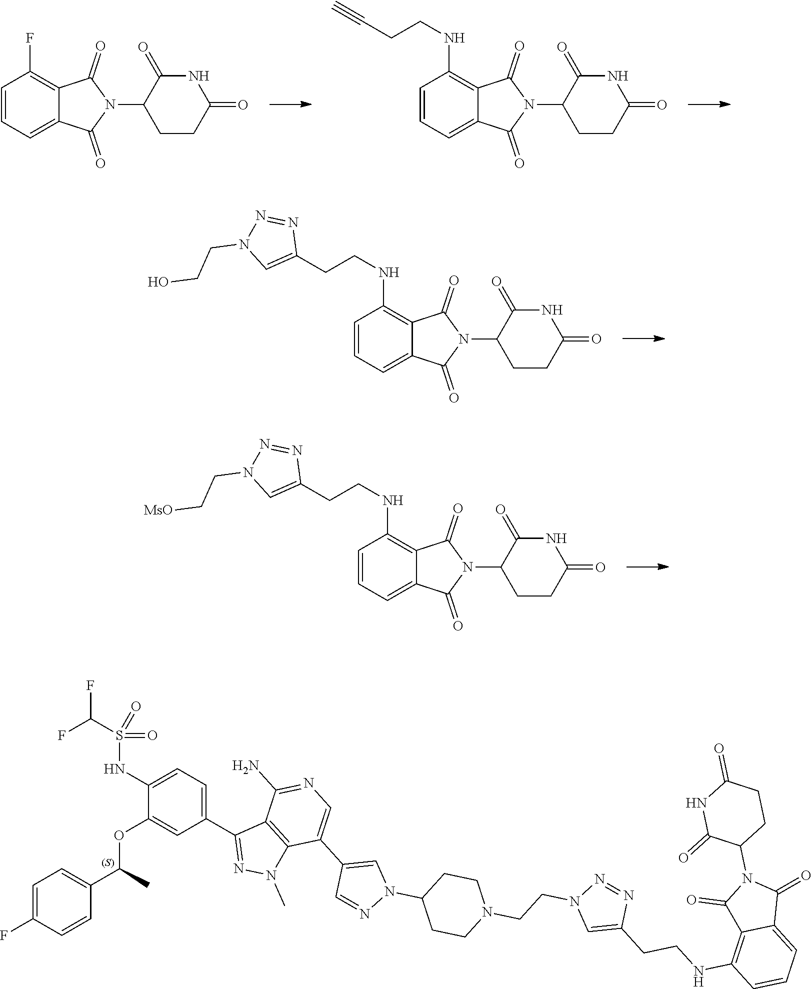

### Step 1: 4-[(but-3-yn-1-yl)amino]-2-(2,6-dioxopiperidin-3-yl)-2,3-dihydro-1H-isoindole-1,3-dione

A mixture of 2-(2,6-dioxopiperidin-3-yl)-4-fluoro-2,3-dihydro-1H-isoindole-1,3-dione (2 g, 7.24 mmol), DIPEA (2.80 g, 21.7 mmol) and but-3-yn-1-amine (500 mg, 7.24 mmol) in dioxane (40 mL) was stirred at 100° C. under N₂ for 16 h. The reaction was concentrated, and the crude residue was purified by column chromatography (PE:EtOAc, 2:1) to afford the title product (1.6 g, 68%) as a yellow solid. LCMS (Method A): 1.96 min, m/z: 326.1 [M+H]⁺.

### Step 2: 2-(2,6-dioxopiperidin-3-yl)-4-({2-[1-(2-hydroxyethyl)-1H-1,2,3-triazol-4-yl]ethyl}amino)-2,3-dihydro-1H-isoindole-1,3-dione

A mixture of 4-[(but-3-yn-1-yl)amino]-2-(2,6-dioxopiperidin-3-yl)-2,3-dihydro-1H-isoindole-1,3-dione (250 mg, 768 μmol), 2-azidoethan-1-ol (66.9 mg, 768 μmol) sodium ascorbate (455 mg, 2.30 mmol) and CuSO₄ (122 mg, 768 μmol) in 50% aq. THE (10 mL) was stirred at RT for 2 h. The mixture was filtered and the filtrate was concentrated, then the residue was purified by prep-TLC (DCM/MeOH=20/1) to give the title product (130 mg 41%) as a yellow solid. LCMS (Method B): 1.33 min, m/z: 413.2 [M+H]⁺.

### Step 3: 2-[4-(2-{[2-(2,6-dioxopiperidin-3-yl)-1,3-dioxo-2,3-dihydro-1H-isoindol-4-yl]amino}ethyl)-1H-1,2,3-triazol-1-yl]ethyl methanesulfonate

To a mixture of 2-(2,6-dioxopiperidin-3-yl)-4-({2-[1-(2-hydroxyethyl)-1H-1,2,3-triazol-4-yl]ethyl}amino)-2,3-dihydro-1H-isoindole-1,3-dione (100 mg, 0.24 mmol) and Et₃N (57.5 mg, 0.73 mmol) in DCM (5 mL) was added MsCl (36.0 mg, 0.32 mmol), and the reaction was stirred at RT overnight. The mixture was poured into water (50 mL) and extracted with EtOAc (3×25 mL). The combined organic layers were washed with brine (25 mL) and concentrated to give the title product (51.0 mg, 43%) as a yellow solid. LCMS (Method A): 1.62 min, m/z: 491.2 [M+H]⁺.

### Step 4: N-(4-{4-amino-7-[1-(1-{2-[4-(2-{[2-(2,6-dioxopiperidin-3-yl)-1,3-dioxo-2,3-dihydro-1H-isoindol-4-yl]amino}ethyl)-1H-1,2,3-triazol-1-yl]ethyl}piperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethane sulfonamide

A mixture of N-(4-{4-amino-1-methyl-7-[1-(piperidin-4-yl)-1H-pyrazol-4-yl]-1H-pyrazolo[4,3-c]pyridin-3-yl}-2-[(1S)-1-(4-fluorophenyl)ethoxy]phenyl)-1,1-difluoromethanesulfonamide 78.3 mg, 0.12 mmol), 2-[4-(2-{[2-(2,6-dioxopiperidin-3-yl)-1,3-dioxo-2,3-dihydro-1H-isoindol-4-yl]amino}ethyl)-1H-1,2,3-triazol-1-yl]ethyl methanesulfonate (60 mg, 0.12 mmol) and Et₃N (37.0 mg, 0.37 mmol) in DMF (3 mL) was stirred at 50° C. under N₂ for 16 h. The reaction was concentrated, and the crude residue was purified by prep-TLC (DCM:MeOH=20/1) to afford the title product (2.5 mg, 2%) as a yellow solid. LCMS (Method A): 1.76 min; m/z: 518.3 [M+2H]²⁺. ¹H NMR (400 MHz, CDCl₃): 11.24 (s, 1H), 10.01 (s, 1H), 7.70 (d, J=8.2 Hz, 1H), 7.57 (d, J=3.4 Hz, 1H), 7.53 (d, J=7.1 Hz, 1H), 7.50-7.48 (m, 1H), 7.32 (dd, J=8.5, 5.3 Hz, 2H), 7.19 (dd, J=8.3, 1.7 Hz, 1H), 7.11-7.04 (m, 2H), 7.01 (d, J=11.5 Hz, 1H), 6.93 (d, J=8.6 Hz, 1H), 6.36 (s, 1H), 6.33 (t, J=6.4 Hz, 1H), 5.47 (d, J=6.4 Hz, 1H), 5.38-5.31 (m, 1H), 4.90 (dd, J=12.0, 5.5 Hz, 1H), 4.52 (dt, J=13.5, 6.7 Hz, 1H), 4.40 (dt, J=13.5, 6.3 Hz, 1H), 4.29-4.16 (m, 1H), 3.78 (s, 2H), 3.69-3.58 (m, 2H), 3.10 (d, J=7.3 Hz, 6H), 2.74 (dd, J=15.7, 4.8 Hz, 2H), 2.34 (t, J=11.5 Hz, 2H), 2.25-2.18 (m, 3H), 2.17-2.13 (m, 2H), 2.03-1.98 (m, 3H), 1.71 (d, J=6.4 Hz, 3H), 1.46 (s, 7H).

### Compound 1135: N-(4-{4-amino-7-[1-(1-{2-[1-(2-{[2-(2,6-dioxopiperidin-3-yl)-1,3-dioxo-2,3-dihydro-1H-isoindol-4-yl]amino}ethyl)-1H-1,2,3-triazol-4-yl]ethyl}piperidin-4-yl)-1H-pyrazol-4-yl]-1-methyl-1H-pyrazolo[4,3-c]pyridin-3-yl}-2-[(1 S)-1-(4-fluorophenyl)ethoxy]phenyl)-1,1-difluoromethanesulfonamide

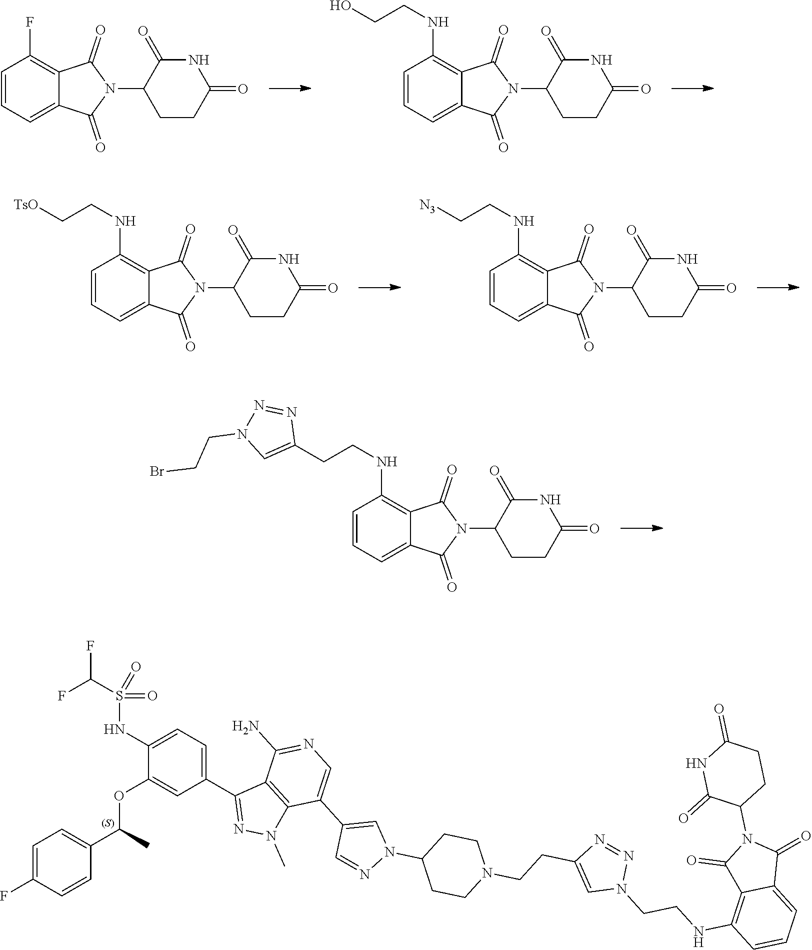

### Step 1: 2-(2,6-dioxopiperidin-3-yl)-4-[(2-hydroxyethyl)amino]-2,3-dihydro-1H-isoindole-1,3-dione

To a solution of 2-(2,6-dioxopiperidin-3-yl)-4-fluoro-2,3-dihydro-1H-isoindole-1,3-dione (500 mg, 1.81 mmol) in DMSO (8 mL) were added 2-aminoethan-1-ol (150 mg, 2.46 mmol) and DIPEA (703 mg, 5.43 mmol), and the mixture was stirred at 90° C. under N₂ for 8 h. The mixture was concentrated, and the residue was purified by reverse-phase chromatography and Prep-TLC (DCM:MeOH=15:1) to afford the title product (193 mg, 34%) as a yellow-green solid. LCMS (Method A): 1.19 min, m/z: 318.1, 340.1 [M+H]⁺.

### Step 2: 2-{[2-(2,6-dioxopiperidin-3-yl)-1,3-dioxo-2,3-dihydro-1H-isoindol-4-yl]amino}ethyl 4-methylbenzene-1-sulfonate

To a mixture of 2-(2,6-dioxopiperidin-3-yl)-4-[(2-hydroxyethyl)amino]-2,3-dihydro-1H-isoindole-1,3-dione (193 mg, 0.61 mmol) and Et₃N (2 mL, 1.98 mmol) in DCM (4 mL) was added TsCl (120 mg, 0.63 mmol, and the reaction was stirred at RT under N₂ for 16 h. The mixture was concentrated, and the residue was purified by column chromatography (DCM:MeOH=15:1) to afford the title product (152 mg, 53%) as a yellow solid. LCMS (Method A): 2.04 min, m/z: 472.2 [M+H]⁺.

### Step 3: 4-[(2-azidoethyl)amino]-2-(2,6-dioxopiperidin-3-yl)-2,3-dihydro-1H-isoindole-1,3-dione

To a solution of 2-{[2-(2,6-dioxopiperidin-3-yl)-1,3-dioxo-2,3-dihydro-1H-isoindol-4-yl]amino}ethyl 4-methylbenzene-1-sulfonate (152 mg, 0.32 mmol) in DMF (4 mL) was added NaN₃ (24 mg, 0.37 mmol), and the reaction was stirred at 80° C. under N₂ for 16 h. The mixture was concentrated, and the residue was purified by column chromatography (DCM:MeOH=20:1) to afford the title product (103 mg, 94%) as a yellow solid. LCMS (Method A): 1.80 min, m/z: 343.2 [M+H]⁺.

### Step 4: 4-({2-[4-(2-bromoethyl)-1H-1,2,3-triazol-1-yl]ethyl}amino)-2-(2,6-dioxopiperidin-3-yl)-2,3-dihydro-1H-isoindole-1,3-dione

A mixture of 4-[(2-azidoethyl)amino]-2-(2,6-dioxopiperidin-3-yl)-2,3-dihydro-1H-isoindole-1,3-dione (103 mg, 0.30 mmol), 4-bromobut-1-yne (40.0 mg, 0.30 mmol), CuSO₄ 5H₂O (200 mg, 0.80 mmol) and sodium ascorbate (200 mg, 1.01 mmol) in 50% aq. THE (4 mL) was stirred at RT for 12 h. The mixture was concentrated, then diluted with water and extracted with DCM. The combined extracts were dried over Na₂SO₄ and concentrated to afford the title product (240 mg, >100%) as a green solid.

### Step 5: N-(4-{4-amino-7-[1-(1-{2-[1-(2-{[2-(2,6-dioxopiperidin-3-yl)-1,3-dioxo-2,3-dihydro-1H-isoindol-4-yl]amino}ethyl)-1H-1,2,3-triazol-4-yl]ethyl}piperidin-4-yl)-1H-pyrazol-4-yl]-1-methyl-1H-pyrazolo[4,3-c]pyridin-3-yl}-2-[(1S)-1-(4-fluorophenyl)ethoxy]phenyl)-1,1-difluoromethanesulfonamide

A mixture of 4-({2-[4-(2-bromoethyl)-1H-1,2,3-triazol-1-yl]ethyl}amino)-2-(2,6-dioxopiperidin-3-yl)-2,3-dihydro-1H-isoindole-1,3-dione (111 mg, 0.23 mmol), N-(4-{4-amino-1-methyl-7-[1-(piperidin-4-yl)-1H-pyrazol-4-yl]-1H-pyrazolo[4,3-c]pyridin-3-yl}-2-[(1 S)-1-(4-fluorophenyl)ethoxy]phenyl)-1,1-difluoromethanesulfonamide (50 mg, 78.0 μmol) and Et₃N (0.5 mL, 4.94 mmol) in MeCN (4 mL) was stirred at 80° C. under N₂ for 12 h. The mixture was concentrated, and the residue was purified by Prep-TLC (DCM:MeOH=10:1) to afford the title product (2.50 mg, 1%) as a green solid. LCMS (Method A): 1.48 min, m/z: 518.3 [M+H]⁺. ¹H NMR (400 MHz, MeOD-d₄): 7.88-7.83 (m, 1H), 7.81-7.75 (m, 1H), 7.60 (s, 1H), 7.53-7.46 (m, 3H), 7.33 (d, J=8.0 Hz, 1H), 7.05-6.97 (m, 3H), 6.90-6.86 (m, 1H), 6.31 (t, J=55.1 Hz, 1H), 5.34 (t, J=4.8 Hz, 1H), 4.62 (q, J=5.3 Hz, 2H), 3.86 (s, 2H), 3.69 (d, J=1.7 Hz, 2H), 3.64 (s, 1H), 3.48 (p, J=1.6 Hz, 1H), 2.87 (dd, J=14.3, 6.2 Hz, 2H), 2.58 (t, J=7.6 Hz, 1H), 2.48 (d, J=7.5 Hz, 1H), 2.38-2.31 (m, 1H), 2.26 (d, J=11.0 Hz, 1H), 2.03 (d, J=6.1 Hz, 2H), 1.65 (d, J=6.4 Hz, 3H), 1.62-1.54 (m, 7H).

### Biology

### In Vitro Assay

Binding affinity of the test compounds for human MLKL (full length), mouse MLKL (full length), human RIPK1 and human RIPK3 was determined using the KINOMEscan™ technology developed by DiscoverX (USA; http://www.discoverx.com). The assay was conducted according to manufacturer instructions.

### Protocol Description

Kinase assays. For most assays, kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 μm) to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions to screen test compounds for kinase binding activity were assembled by combining kinases, liganded affinity beads, and test compounds in 1× binding buffer (20% SeaBlock, 0.17×PBS, 0.05% Tween 20, 6 mM DTT). All reactions were performed in polypropylene 384-well plates in a final volume of 20 μL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

### Compound Handling

An 11-point 3-fold serial dilution of each test compound was prepared in 100% DMSO at 100× final test concentration and subsequently diluted to 1× in the assay (final DMSO concentration=1%). Most Kos were determined using a compound top concentration=30,000 nM. If the initial K_{D} determined was <0.5 nM (the lowest concentration tested in the initial serial dilution), the measurement was repeated with a further 11 point 3-fold serial dilution starting at 3,000 nM.

### Binding Constants (K_{D}s)

K_{D} for each test compound was calculated with a standard dose-response curve using the Hill equation (equation (1)):

$$Response = Background + \frac{Signal - Background}{1 + ( {K}_{D}^{HillSlope} / {Dose}^{HillSlope}} \qquad ( 1 )$$

The Hill Slope was set to −1.

Curves were fitted using a non-linear least square fit with the Levenberg-Marquardt algorithm.

### REFS

Fabian, M. A. et al. A small molecule-kinase interaction map for clinical kinase inhibitors. Nat. Biotechnol. 23, 329-336 (2005).
Karaman, M. W. et al. A quantitative analysis of kinase inhibitor selectivity. Nat. Biotechnol. 26, 127-132 (2008).
Hill, A. V. The possible effects of the aggregation of the molecules of hemoglobin on its dissociation curves. J. Physiol. (Lond.) 40, iv-vii (1910).
Levenberg, K. A method for the solution of certain non-linear problems in least squares. Q. Appl. Math. 2, 164-168 (1944).

The results of the in vitro screening of the compounds described above are shown below in Tables 21 and 22.

TABLE 21
Results of binding assay for compounds of the
invention against human MLKL, human
RIPK1 and human RIPK3. Binding affinity is
measured by the equilibrium dissociation constant
(K_{D}). The smaller the K_{D} value, the greater the binding
affinity of the ligand for its target. Activity is provided as follows:

| Compound | Human MLKL | Human | Human |
|---|---|---|---|
| ID | (full-length) (K_{D}) | RIPK1 (K_{D}) | RIPK3 (K_{D}) |
| 1 | +++ | + | + |
| 2 | +++ | + | + |
| 3 | ++++ | + | + |
| 4 | +++ | + | + |
| 5 | ++++ | + | + |
| 6 | ++++ | + | + |
| 7 | +++ | + | + |
| 8 | ++++ | + | + |
| 9 | ++++ | + | + |
| 10 | +++ | + | + |
| 11 | +++ | + | + |
| 12 | ++++ | + | ++ |
| 13 | ++++ | + | + |
| 14 | ++++ | + | + |
| 15 | ++++ | + | + |
| 16 | ++++ | + | + |
| 17 | ++++ | + | + |
| 18 | ++++ | + | + |
| 19 | ++++ | + | + |
| 20 | ++++ | + | + |
| 21 | ++++ | + | + |
| 22 | ++++ | + | + |
| 23 | +++ | + | + |
| 24 | ++++ | + | + |
| 25 | ++++ | + | + |
| 26 | ++++ | + | + |
| 27 | +++ | + | + |
| 28 | ++++ | + | + |
| 29 | ++++ | + | + |
| 30 | +++ | + | ++ |
| 31 | ++++ | + | + |
| 32 | +++ | ++ | + |
| 33 | +++ | + | + |
| 34 | +++ | + | + |
| 35 | ++++ | + | + |
| 36 | ++++ | + | + |
| 37 | ++++ | + | + |
| 38 | ++++ | + | + |
| 39 | ++++ | + | + |
| 40 | ++++ | + | + |
| 41 | ++++ | + | + |
| 42 | ++ | + | + |
| 43 | +++ | + | + |
| 44 | +++ | + | + |
| 45 | +++ | + | ++ |
| 46 | +++ | + | + |
| 47 | +++ | + | ++ |
| 48 | +++ | + | + |
| 49 | +++ | + | + |
| 50 | ++++ | + | + |
| 51 | +++ | + | + |
| 52 | +++ | + | + |
| 53 | ++++ | ++ | + |
| 54 | +++ | + | + |
| 55 | ++++ | + | + |
| 56 | ++++ | + | + |
| 57 | +++ | + | ++ |
| 58 | ++++ | + | + |
| 59 | ++++ | + | + |
| 60 | +++ | + | + |
| 61 | +++ | + | + |
| 62 | ++ | ++ | + |
| 63 | ++ | + | + |
| 64 | ++ | + | + |
| 65 | +++ | + | + |
| 66 | ++++ | + | + |
| 67 | ++++ | + | + |
| 68 | ++ | + | + |
| 69 | ++++ | + | + |
| 70 | ++++ | + | + |
| 71 | +++ | ++ | + |
| 72 | ++++ | + | + |
| 73 | ++++ | + | + |
| 74 | ++++ | + | + |
| 75 | ++++ | + | + |
| 76 | ++++ | + | + |
| 77 | ++++ | + | + |
| 78 | ++++ | + | + |
| 79 | ++++ | + | + |
| 80 | ++++ | + | + |
| 81 | ++++ | + | + |
| 82 | ++++ | + | + |
| 83 | +++ | + | + |
| 84 | +++ | + | + |
| 85 | +++ | + | + |
| 86 | +++ | + | + |
| 87 | +++ | + | + |
| 88 | ++++ | + | + |
| 89 | ++++ | + | + |
| 90 | +++ | + | + |
| 91 | ++ | + | + |
| 92 | ++++ | + | + |
| 93 | ++++ | + | + |
| 94 | ++++ | + | + |
| 95 | +++ | + | + |
| 96 | ++++ | + | + |
| 97 | ++++ | + | + |
| 98 | ++++ | + | + |
| 99 | ++++ | + | + |
| 100 | ++++ | + | + |
| 101 | ++++ | + | + |
| 102 | ++++ | + | + |
| 103 | ++++ | + | + |
| 104 | ++++ | + | + |
| 105 | ++++ | + | + |
| 106 | +++ | + | + |
| 107 | ++++ | + | + |
| 108 | ++++ | + | + |
| 109 | ++++ | + | + |
| 110 | ++++ | + | + |
| 111 | +++ | + | + |
| 112 | +++ | + | + |
| 113 | ++++ | + | + |
| 114 | +++ | + | + |
| 115 | +++ | + | + |
| 116 | +++ | + | + |
| 117 | +++ | + | + |
| 118 | +++ | + | + |
| 119 | +++ | + | + |
| 120 | +++ | + | + |
| 121 | ++++ | + | + |
| 122 | ++++ | + | + |
| 123 | ++++ | + | + |
| 124 | ++ | + | + |
| 125 | +++ | + | + |
| 126 | +++ | + | + |
| 127 | +++ | + | + |
| 128 | +++ | + | + |
| 129 | ++++ | + | + |
| 130 | ++++ | + | + |
| 131 | +++ | + | + |
| 132 | ++++ | + | + |
| 133 | ++++ | + | + |
| 134 | +++ | + | + |
| 135 | ++++ | + | + |
| 136 | nd | nd | nd |
| 137 | nd | nd | nd |
| 138 | +++ | + | + |
| 139 | +++ | + | + |
| 140 | +++ | + | + |
| 141 | +++ | + | + |
| 142 | ++++ | + | + |
| 143 | ++++ | + | + |
| 144 | ++++ | + | + |
| 145 | +++ | + | + |
| 146 | +++ | + | + |
| 147 | +++ | + | + |
| 148 | +++ | + | + |
| 149 | nd | nd | nd |
| 150 | ++++ | + | + |
| 151 | ++++ | + | + |
| 152 | nd | nd | nd |
| 153 | +++ | + | + |
| 154 | ++++ | + | + |
| 155 | +++ | + | + |
| 156 | +++ | + | + |
| 157 | +++ | + | + |
| 158 | +++ | + | + |
| 159 | ++++ | + | + |
| 160 | ++++ | + | + |
| 161 | ++++ | + | + |
| 162 | ++++ | + | + |
| 163 | ++++ | + | + |
| 164 | ++++ | + | + |
| 165 | +++ | + | + |
| 166 | +++ | + | + |
| 167 | +++ | + | + |
| 168 | ++++ | + | + |
| 169 | nd | nd | nd |
| 170 | +++ | + | + |
| 171 | +++ | + | + |
| 172 | +++ | ++ | + |

++++ = K_{D} < 1 nM
+++ = 1 nM < K_{D} < 100 nM
++ = 100 nM < K_{D} < 10,000 nM
+ = K_{D} > 10,000 nM

TABLE 22
Results of binding assay for compounds of the invention
against mouse MLKL. Binding affinity is measured by the
equilibrium dissociation constant (K_{D}). The smaller the K_{D}
value, the greater the binding affinity of the ligand for its target.

| Compound ID | Mouse MLKL(full-length) (K_{D}) |
|---|---|
| Compound 5 | ++++ |
| Compound 7 | ++++ |
| Compound 8 | ++++ |
| Compound 9 | +++ |
| Compound 11 | +++ |
| Compound 12 | ++++ |
| Compound 13 | ++++ |
| Compound 15 | ++++ |
| Compound 18 | +++ |
| Compound 19 | ++++ |
| Compound 20 | ++++ |
| Compound 60 | ++++ |
| Compound 61 | +++ |
| Compound 66 | +++ |
| Compound 69 | +++ |
| Compound 70 | +++ |
| Compound 72 | +++ |

Activity is provided as follows:
++++ = K_{D} < 1 nM
+++ = 1 nM < K_{D} < 100 nM
++ = 100 nM < K_{D} < 10,000 nM
+ = K_{D} > 10,000 nM

### Cellular Assay: Screening Compounds for Inhibition of TSQ Induced Necroptosis, 384 Well Plate Format.

Cell Line ID: U937 human histiocytic leukemia cell line.
Cell Concentration (cells/well): Final cell density is 20000 cells per well.
Cell growth medium: HT-RPMI medium+7.4% Fetal Bovine Serum (FBS). Cells are cultured in Corning 150 cm² tissue culture flasks with vented caps at 37° C./5% CO₂.
Incubation: Plates were incubated at 37° C./5% CO₂ in a humidified incubator for 48 hours following addition of compounds and death stimuli (TSQ cocktail).
Compound concentration: 36 μM starting concentration, 1:3 dilution, 10 point
DMSO final concentration (% v/v): 0.3%.
   Compounds in TSQ Cocktail (T: TNF; S: Smac Mimetic; Q: Q-VD-OPh) and their Final Concentrations:
hTNF-Fc (100 ng/ml)—produced by standard procedures as shown in Bossen et al., J. Biol. Chem., 2006, 281(20), 13964-13971.
Compound A (500 nM)—Smac mimetic, Tetralogic and SYNthesis med chem
Q-VD-OPh (10 μM)—MP Biomedicals

### Assay Experimental Outline

The cellular assay was carried out according to the following steps:
1. Each well was prepared by sequential addition of:
   a. DMSO (control; columns 1-2 and 23-24) or compound in DMSO—addition was performed using acoustic transfer of nL volumes of stock compound to give final test concentrations of 36, 12, 4, 1.3, 0.44, 0.148, 0.049, 0.016, 0.005 and 0.002 μM. All wells were backfilled with DMSO to a final total volume in the well of 100 nL.
   b. Following compound/DMSO addition in step (a), 40 μL of cell suspension (5×10⁵ cells/mL) was added to provide a final cell concentration of 20,000 cells per well, and
   c. Following cell addition in step (b), 10 μL of 5×TSQ cocktail (except to positive controls; columns 1 and 23) was added to each well.
2. After 48 hours, plate was removed from the 37° C. incubator and equilibrated to room temperature for 45 minutes.
3.15 μL of room temperature CellTitre-Glo2™ (Promega™) was added to each well.
4. Shook plates for 2 minutes (˜600 rpm) and incubated at room temperature for 15 minutes to allow signal to stabilise.
5. Read luminescence readout on a plate reader.

### Analysis:

Percent viability was calculated for each compound according to equation (2):

$$\% viability = 100 \times ( ( RawData - NSA ) / ( TA - NSA ) \qquad ( 2 )$$

wherein
RawData is the readout of any cell containing a compound of the invention.
TA is the total activity provided by the luminescence readout from DMSO only wells (columns 2 and 24)=100% viability.
NSA is the non-specific activity provided by DMSO+TSQ wells (columns 1 and 23)=0% viability
Curve fitting: 10-point titration curves are fitted with the 4-parameter logistic nonlinear regression model and the IC₅₀ reported is the inflection point of the curve
Analysis: Data was loaded into Dotmatics™ and visualised using the Tibco@Spotfire™ software point titration curves were fitted with the 4 parameter logistic nonlinear regression model and the IC₅₀ reported reflect the inflection point of the curve for curve fitting.

### Interpretation of Results:

Assay involving the TSQ cocktail (T: TNF; S: Smac mimetic; Q: Q-VD-OPh): TSQ treatment ensures that cells specifically undergo necroptotic cell death. TNF activates the TNF receptor, Smac mimetic directs the signal away from proinflammatory signaling and toward the RIPK1/RIPK3-mediated cell death pathways, and Q-VD-OPh ensures that the apoptotic response is blocked leaving only the programmed necrosis response. The compounds' activity (solution in DMSO) tested in this TSQ-induced assay was evaluated by determining the number of viable cells in culture by measuring the amount of ATP present as measured by CelltiterGlo.
Counter screen: In parallel, all compounds were tested for their ability to affect cell viability. The same U937 cells were treated with compound in DMSO without the TSQ cocktail. This counter screen enabled evaluation of off-target effects. In this case, cell viability was measured by CelltiterGlo.

The results of the cellular screening of the compounds described above are shown below in Table 23.

TABLE 23
Table showing the results of cell-based assays performed,
analysed as the half maximal inhibitory concentration
(IC₅₀), being the concentration of the test compound needed
to inhibit TSQ induced necroptosis by 50%. The lower
the IC₅₀, the more potent the compound.

| | Inhibition of TSQ- | Off-target effect |
|---|---|---|
| | induced necroptosis | (IC₅₀ |
| Compound No | (IC₅₀ cell-based assay) | cell-based assay) |
| Compound 1 | ++ | + |
| Compound 7 | +++ | ++ |
| Compound 9 | +++ | ++ |
| Compound 12 | ++ | + |
| Compound 13 | +++ | + |
| Compound 14 | ++ | + |
| Compound 15 | +++ | + |
| Compound 16 | ++ | + |
| Compound 18 | +++ | + |
| Compound 19 | ++++ | + |
| Compound 20 | +++ | + |
| Compound 21 | ++ | + |
| Compound 23 | ++ | + |
| Compound 24 | +++ | + |
| Compound 25 | +++ | + |
| Compound 26 | +++ | + |
| Compound 27 | +++ | + |
| Compound 28 | +++ | + |
| Compound 29 | ++++ | + |
| Compound 30 | ++++ | + |
| Compound 31 | ++++ | + |
| Compound 32 | ++ | + |
| Compound 35 | +++ | + |
| Compound 36 | ++++ | + |
| Compound 37 | +++ | + |
| Compound 38 | ++++ | + |
| Compound 39 | +++ | + |
| Compound 40 | ++++ | + |
| Compound 41 | ++++ | + |
| Compound 42 | ++ | + |
| Compound 43 | +++ | + |
| Compound 44 | +++ | + |
| Compound 45 | +++ | + |
| Compound 46 | +++ | + |
| Compound 48 | ++ | ++ |
| Compound 50 | +++ | + |
| Compound 51 | +++ | + |
| Compound 52 | +++ | + |
| Compound 55 | ++ | + |
| Compound 56 | +++ | + |
| Compound 57 | +++ | + |
| Compound 58 | +++ | + |
| Compound 59 | +++ | + |
| Compound 60 | +++ | + |
| Compound 61 | +++ | + |
| Compound 62 | ++ | + |
| Compound 72 | +++ | + |
| Compound 73 | +++ | + |
| Compound 74 | +++ | + |
| Compound 75 | ++++ | + |
| Compound 76 | ++++ | + |
| Compound 77 | ++++ | + |
| Compound 78 | +++ | + |
| Compound 79 | +++ | + |
| Compound 80 | +++ | + |
| Compound 81 | ++++ | + |
| Compound 82 | +++ | + |
| Compound 83 | +++ | + |
| Compound 84 | ++ | + |
| Compound 85 | ++ | + |
| Compound 86 | +++ | + |
| Compound 87 | ++ | + |
| Compound 88 | +++ | + |
| Compound 89 | +++ | + |
| Compound 92 | +++ | + |
| Compound 93 | +++ | + |
| Compound 94 | +++ | + |
| Compound 95 | +++ | + |
| Compound 96 | +++ | + |
| Compound 97 | +++ | + |
| Compound 98 | +++ | + |
| Compound 99 | +++ | + |
| Compound 100 | ++++ | + |
| Compound 101 | ++++ | + |
| Compound 102 | +++ | + |
| Compound 103 | ++++ | + |
| Compound 104 | ++++ | + |
| Compound 105 | +++ | + |
| Compound 106 | +++ | + |
| Compound 107 | +++ | + |
| Compound 108 | +++ | + |
| Compound 109 | ++++ | + |
| Compound 111 | +++ | + |
| Compound 113 | ++++ | + |
| Compound 115 | +++ | + |
| Compound 116 | ++++ | + |
| Compound 117 | +++ | + |
| Compound 119 | +++ | + |
| Compound 120 | ++++ | + |
| Compound 121 | +++ | + |
| Compound 122 | +++ | + |
| Compound 123 | ++++ | + |
| Compound 126 | +++ | + |
| Compound 128 | +++ | + |
| Compound 129 | +++ | + |
| Compound 131 | +++ | + |
| Compound 132 | +++ | + |
| Compound 133 | +++ | + |
| Compound 134 | +++ | + |
| Compound 135 | +++ | + |
| Compound 136 | +++ | + |
| Compound 137 | ++++ | + |
| Compound 138 | +++ | + |
| Compound 139 | +++ | + |
| Compound 140 | +++ | + |
| Compound 141 | +++ | + |
| Compound 142 | +++ | + |
| Compound 145 | ++++ | + |
| Compound 146 | ++++ | + |
| Compound 147 | +++ | + |
| Compound 148 | ++++ | + |
| Compound 149 | ++++ | + |
| Compound 152 | ++++ | + |
| Compound 153 | +++ | + |
| Compound 154 | ++++ | + |
| Compound 155 | ++++ | + |
| Compound 156 | +++ | + |
| Compound 157 | +++ | + |
| Compound 158 | ++++ | + |
| Compound 159 | ++++ | + |
| Compound 160 | +++ | + |
| Compound 161 | ++ | + |
| Compound 162 | ++++ | + |
| Compound 163 | +++ | + |
| Compound 164 | ++++ | + |
| Compound 165 | +++ | + |
| Compound 166 | +++ | + |
| Compound 167 | +++ | ++ |
| Compound 168 | +++ | + |
| Compound 170 | +++ | + |
| Compound 171 | +++ | + |
| Compound 172 | +++ | + |
| Compound 173 | ++ | + |

Activity is provided as follows:
++++ = IC₅₀ < 10 nM
+++ = 10 nM < IC₅₀ < 1,000 nM
++ = 1,000 nM < IC₅₀ < 10,000 nM
+ = IC₅₀ > 10,000 nM

### Cellular Assay: Screening Compounds for Inhibition of TSI Induced Necroptosis in, 384 Well Plate Format.

Cell Line ID: Immortalised Mouse Dermal Fibroblasts (MDFs).
Cell Concentration (cells/well): Final cell density is 2200 cells per well.
Cell growth medium: DMEM medium+7.4% Fetal Bovine Serum (FBS). Cells are cultured in Corning 150 cm² tissue culture flasks with vented caps at 37° C./10% CO₂.
Incubation: Plates were incubated for 30 minutes following addition of compounds and then 3 hours incubation with death stimuli (TSI cocktail) at 37° C./10% CO₂ in a humidified incubator.
Compound concentration: 36 μM starting concentration, 1:3 dilution, 10 point
DMSO final concentration (% v/v): 0.3%.
   Compounds in TSI Cocktail (T: TNF; S: Smac Mimetic; I: IDN-6556) and their Final Concentrations:
rhTNF (100 ng/ml)
Compound a (500 nM)—Smac Mimetic, Tetralogic and SYNthesis Med Chem
IDN-6556 (10 μM)—Selleckchem (S7775)

### Assay Experimental Outline

The cellular assay was carried out according to the following steps:
1. Each well was prepared by sequential addition of:
a. 40 μL of cell suspension (5.5×10⁴ cells/mL) was added to each well to provide a final cell concentration of 2,200 cells per well.
b. After 24 hours, DMSO (control; columns 1-2 and 23-24) or compound in DMSO—addition was performed using acoustic transfer of nL volumes of stock compound to give final test concentrations of 36, 12, 4, 1.3, 0.44, 0.148, 0.049, 0.016, 0.005 and 0.002 μM. All wells were backfilled with DMSO to a final total volume in the well of 100 nL.
c. Following compound/DMSO addition in step (b), Plates were incubated for 30 minutes at 37° C./10% CO₂ in a humidified incubator and then 10 μL of 5×TSI cocktail (except to positive controls; columns 2 and 24) was added to each well.
2. After 3 hours, plate was removed from the 37° C. incubator and equilibrated to room temperature for 15 minutes.
3. 15 μL of room temperatured CellTitre-Glo2™ (Promega™) was added to each well.
4. Shook plates for 2 minutes (˜600 rpm) and incubated at room temperature for 15 minutes to allow signal to stabilise.
5. Read luminescence readout on a plate reader.

### Analysis:

Percent viability was calculated for each compound according to equation (2):

$$\% viability = 100 \times ( ( RawData - NSA ) / ( TANSA ) \qquad ( 2 )$$

Wherein RawData is the readout of any cell containing a compound of the invention.

TA is the total activity provided by the luminescence readout from DMSO only wells (columns 2 and 24)=100% viability.

NSA is the non-specific activity provided by DMSO+TSI wells (columns 1 and 23)=0% viability
Curve fitting: 10-point titration curves are fitted with the 4-parameter logistic nonlinear regression model and the IC₅₀ reported is the inflection point of the curve
Analysis: Data was loaded into Dotmatics™ and visualised using the Tibco@
Spotfire™ software. 10 point titration curves were fitted with the 4 parameter logistic nonlinear regression model and the IC₅₀ reported reflect the inflection point of the curve for curve fitting.

### Interpretation of Results:

Assay involving the TSI cocktail (T: TNF; S: Smac mimetic; I: IDN-6556): TSI treatment ensures that cells specifically undergo necroptotic cell death. TNF activates the TNF receptor, Smac mimetic directs the signal away from proinflammatory signaling and toward the RIPK1/RIPK3-mediated cell death pathways, and IDN-6556 (Caspase inhibitor) ensures that the apoptotic response is blocked leaving only the programmed necrosis response. The compounds' activity (solution in DMSO) tested in this TSI-induced assay was evaluated by determining the number of viable cells in culture by measuring the amount of ATP present as measured by CelltiterGlo.

The results of the cellular screening of the compounds described above are shown below in Table 24.

TABLE 24
Table showing the results of cell-based assays
performed, analysed as the half maximal
inhibitory concentration (IC₅₀), being the
concentration of the test compound needed to
inhibit TSI induced necroptosis by 50%. The lower
the IC₅₀, the more potent the compound.

| | Inhibition of TSI-induced necroptosis |
|---|---|
| Compound No | (IC₅₀ cell-based assay) in MDF |
| Compound 3 | ++ |
| Compound 11 | ++ |
| Compound 13 | ++ |
| Compound 19 | +++ |
| Compound 29 | ++ |
| Compound 30 | ++ |
| Compound 46 | +++ |
| Compound 79 | ++ |
| Compound 80 | +++ |
| Compound 81 | ++ |
| Compound 97 | ++ |
| Compound 98 | ++ |
| Compound 99 | ++ |
| Compound 103 | ++ |
| Compound 104 | +++ |
| Compound 109 | +++ |
| Compound 113 | +++ |
| Compound 116 | ++ |
| Compound 120 | ++ |
| Compound 121 | ++ |
| Compound 122 | ++ |
| Compound 131 | ++ |
| Compound 132 | ++ |
| Compound 137 | ++ |
| Compound 139 | ++ |
| Compound 142 | ++ |
| Compound 145 | ++++ |
| Compound 146 | ++ |
| Compound 147 | +++ |
| Compound 148 | +++ |
| Compound 149 | +++ |
| Compound 150 | ++ |
| Compound 152 | +++ |
| Compound 153 | +++ |
| Compound 154 | ++++ |
| Compound 155 | +++ |
| Compound 157 | ++ |
| Compound 158 | ++++ |
| Compound 159 | ++++ |
| Compound 162 | ++++ |
| Compound 163 | ++ |
| Compound 164 | ++++ |
| Compound 166 | ++ |
| Compound 167 | ++++ |
| Compound 168 | +++ |
| Compound 170 | ++++ |
| Compound 171 | ++++ |
| Compound 172 | ++++ |

Activity is provided as follows:
+++ = IC₅₀ < 1,000 nM
++ = 1,000 nM < IC₅₀ < 10,000 nM

TABLE 25
Results of binding assay for compounds of the
invention against hMLKL. Binding affinity
is measured by the equilibrium dissociation constant
(K_{D}). The smaller the K_{D} value, the greater
the binding affinity of the ligand for its target.

| Compound No | MLKL (K_{D}) |
|---|---|
| 1001 | ++++ |
| 1002 | ++++ |
| 1003 | ++++ |
| 1004 | ++++ |
| 1005 | ++++ |
| 1006 | ++++ |
| 1008 | ++++ |
| 1009 | ++++ |
| 1007 | +++ |
| 1010 | ++++ |
| 1011 | nt |
| 1012 | ++++ |
| 1016 | nt |
| 1017 | ++++ |
| 1018 | ++++ |
| 1014 | nt |
| 1015 | ++++ |
| 1020 | +++ |
| 1019 | +++ |
| 1013 | ++++ |
| 1021 | ++++ |
| 1022 | ++++ |
| 1030 | +++ |
| 1029 | nt |
| 1023 | nt |
| 1031 | +++ |
| 1027 | nt |
| 1028 | nt |
| 1025 | nt |
| 1026 | +++ |
| 1024 | ++++ |
| 1037 | +++ |
| 1039 | +++ |
| 1040 | ++++ |
| 1041 | +++ |
| 1042 | +++ |
| 1043 | +++ |
| 1044 | ++++ |
| 1045 | +++ |
| 1046 | nt |
| 1047 | ++++ |
| 1048 | ++++ |
| 1049 | +++ |
| 1050 | +++ |
| 1051 | +++ |
| 1052 | +++ |
| 1053 | +++ |
| 1054 | +++ |
| 1055 | +++ |
| 1056 | +++ |
| 1057 | +++ |
| 1058 | +++ |
| 1059 | ++++ |
| 1060 | ++++ |
| 1061 | ++++ |
| 1062 | ++++ |
| 1063 | ++++ |
| 1064 | +++ |
| 1065 | ++++ |
| 1066 | ++++ |
| 1067 | ++++ |
| 1068 | +++ |
| 1069 | +++ |
| 1070 | +++ |
| 1071 | ++++ |
| 1072 | ++++ |
| 1073 | +++ |
| 1074 | +++ |
| 1075 | ++++ |
| 1076 | ++++ |
| 1077 | nt |
| 1078 | ++++ |
| 1079 | ++++ |
| 1080 | ++++ |
| 1081 | ++++ |
| 1082 | ++++ |
| 1083 | ++++ |
| 1084 | ++++ |
| 1085 | ++++ |
| 1086 | ++++ |
| 1087 | +++ |
| 1088 | ++++ |
| 1089 | ++++ |
| 1090 | ++++ |
| 1091 | ++++ |
| 1092 | ++++ |
| 1093 | +++ |
| 1094 | ++++ |
| 1095 | +++ |
| 1096 | ++++ |
| 1097 | ++++ |
| 1098 | ++++ |
| 1099 | ++++ |
| 1100 | nt |
| 1101 | ++++ |
| 1102 | ++++ |
| 1103 | ++++ |
| 1104 | nt |
| 1105 | +++ |
| 1106 | ++++ |
| 1107 | +++ |
| 1108 | ++++ |
| 1109 | ++++ |
| 1110 | ++++ |
| 1111 | +++ |
| 1112 | ++++ |
| 1113 | ++++ |
| 1114 | ++++ |
| 1115 | +++ |
| 1116 | ++++ |
| 1117 | ++++ |
| 1118 | nt |
| 1119 | ++++ |
| 1120 | ++++ |
| 1121 | ++++ |
| 1122 | ++++ |
| 1123 | ++++ |
| 1124 | nt |
| 1125 | +++ |
| 1126 | ++++ |
| 1127 | +++ |
| 1128 | nt |
| 1129 | +++ |
| 1130 | +++ |
| 1131 | ++++ |
| 1132 | +++ |
| 1133 | +++ |
| 1134 | ++++ |
| 1135 | nt |
| 1136 | ++++ |
| 1137 | nt |
| 1138 | +++ |
| 1139 | nt |
| 1140 | nt |
| 1141 | ++++ |
| 1142 | nt |
| 1143 | nt |
| 1144 | nt |
| 1145 | nt |

Activity is provided as follows:
++++ = K_{D} < 10 nM
+++ = 10 nM < K_{D} < 1,000 nM
++ = 1,000 nM < K_{D} < 30,000 nM
+ = K_{D} > 30,000 nM

TABLE 26
Table showing the results of cell-based assays performed,
analysed as the half maximal inhibitory concentration
(IC₅₀), being the concentration of the test compound
needed to inhibit TSQ induced necroptosis by 50%.
The lower the IC₅₀, the more potent the compound.

| | Inhibition of | Off-target |
|---|---|---|
| | TSQ-induced | effect |
| Compound | necroptosis (IC₅₀ | (IC₅₀ cell- |
| No | cell-based assay) | based assay) |
| 1001 | * | * |
| 1002 | * | * |
| 1003 | * | * |
| 1004 | * | * |
| 1005 | ** | * |
| 1006 | *** | ** |
| 1008 | *** | ** |
| 1009 | *** | ** |
| 1007 | *** | * |
| 1010 | ** | * |
| 1011 | ** | * |
| 1012 | ** | * |
| 1016 | *** | * |
| 1017 | ** | * |
| 1018 | *** | * |
| 1014 | *** | * |
| 1015 | *** | * |
| 1020 | *** | * |
| 1019 | ** | * |
| 1013 | ** | * |
| 1021 | ** | * |
| 1022 | *** | * |
| 1030 | *** | * |
| 1029 | *** | * |
| 1023 | *** | * |
| 1031 | *** | |
| 1027 | +++ | +++ |
| 1028 | +++ | +++ |
| 1025 | ++ | + |
| 1026 | +++ | ++ |
| 1024 | +++ | ++ |
| 1037 | ++ | ++ |
| 1039 | ++ | + |
| 1040 | ++ | + |
| 1041 | ++ | ++ |
| 1042 | +++ | ++ |
| 1043 | +++ | + |
| 1044 | +++ | + |
| 1045 | +++ | + |
| 1046 | +++ | ++ |
| 1047 | +++ | ++ |
| 1048 | +++ | + |
| 1049 | +++ | ++ |
| 1050 | +++ | + |
| 1051 | +++ | + |
| 1052 | ++ | + |
| 1053 | +++ | + |
| 1054 | +++ | + |
| 1055 | +++ | + |
| 1056 | +++ | ++ |
| 1057 | +++ | ++ |
| 1058 | +++ | + |
| 1059 | +++ | + |
| 1060 | ++ | + |
| 1061 | +++ | ++ |
| 1062 | +++ | + |
| 1063 | +++ | + |
| 1064 | +++ | + |
| 1065 | + | + |
| 1066 | ++ | + |
| 1067 | +++ | ++ |
| 1068 | +++ | ++ |
| 1069 | +++ | ++ |
| 1070 | +++ | + |
| 1071 | +++ | + |
| 1072 | +++ | + |
| 1073 | +++ | + |
| 1074 | +++ | + |
| 1075 | +++ | ++ |
| 1076 | +++ | + |
| 1077 | +++ | + |
| 1078 | +++ | + |
| 1079 | +++ | + |
| 1080 | ++ | + |
| 1081 | +++ | + |
| 1082 | ++ | + |
| 1083 | +++ | + |
| 1084 | ++ | + |
| 1085 | +++ | + |
| 1086 | +++ | + |
| 1087 | ++ | + |
| 1088 | +++ | + |
| 1089 | + | + |
| 1090 | ++ | + |
| 1091 | +++ | + |
| 1092 | +++ | + |
| 1093 | +++ | + |
| 1094 | +++ | + |
| 1095 | +++ | + |
| 1096 | +++ | + |
| 1097 | +++ | + |
| 1098 | +++ | + |
| 1099 | +++ | + |
| 1100 | +++ | + |
| 1101 | ++ | + |
| 1102 | +++ | + |
| 1103 | +++ | + |
| 1104 | ++ | ++ |
| 1105 | +++ | + |
| 1106 | +++ | + |
| 1107 | +++ | + |
| 1108 | +++ | + |
| 1109 | +++ | + |
| 1110 | +++ | + |
| 1111 | +++ | + |
| 1112 | +++ | + |
| 1113 | ++ | + |
| 1114 | ++ | + |
| 1115 | +++ | + |
| 1116 | +++ | + |
| 1117 | +++ | + |
| 1118 | + | + |
| 1119 | +++ | + |
| 1120 | +++ | + |
| 1121 | +++ | + |
| 1122 | ++ | + |
| 1123 | +++ | + |
| 1124 | +++ | + |
| 1125 | +++ | + |
| 1126 | +++ | + |
| 1127 | +++ | + |
| 1128 | +++ | + |
| 1129 | +++ | + |
| 1130 | +++ | + |
| 1131 | ++ | + |
| 1132 | +++ | + |
| 1133 | +++ | + |
| 1134 | +++ | + |
| 1135 | +++ | + |
| 1136 | +++ | + |
| 1137 | +++ | + |
| 1138 | +++ | + |
| 1141 | +++ | + |

Activity is provided as follows:
*** = IC₅₀ < 1 uM
** = 1 uM < IC₅₀ < 10 uM
* = IC₅₀ > 10 uM

TABLE 27
Table showing the results of cell-based assays performed,
analysed as the half maximal inhibitory concentration (IC₅₀),
being the concentration of the test compound needed to
inhibit TSI induced necroptosis by 50%. The lower the
IC₅₀, the more potent the compound.

| Compound | Inhibition of TSI-induced necroptosis |
|---|---|
| No | (IC₅₀ cell-based assay) in MDF |
| 1006 | ++ |
| 1008 | +++ |
| 1016 | +++ |
| 1018 | ++ |
| 1014 | ++ |
| 1015 | ++ |
| 1031 | +++ |
| 1026 | ++ |
| 1042 | ++ |
| 1043 | +++ |
| 1044 | +++ |
| 1047 | +++ |
| 1048 | +++ |
| 1049 | ++ |
| 1051 | ++ |
| 1053 | +++ |
| 1054 | +++ |
| 1055 | +++ |
| 1057 | ++ |
| 1058 | +++ |
| 1061 | ++ |
| 1062 | ++ |
| 1064 | ++ |
| 1066 | ++ |
| 1067 | +++ |
| 1068 | ++ |
| 1069 | +++ |
| 1070 | +++ |
| 1071 | +++ |
| 1072 | ++ |
| 1073 | +++ |
| 1074 | +++ |
| 1076 | +++ |
| 1077 | ++ |
| 1078 | +++ |
| 1079 | +++ |
| 1081 | +++ |
| 1082 | ++ |
| 1083 | +++ |
| 1084 | ++ |
| 1085 | +++ |
| 1086 | ++ |
| 1087 | +++ |
| 1088 | +++ |
| 1090 | ++ |
| 1091 | +++ |
| 1093 | +++ |
| 1094 | +++ |
| 1095 | +++ |
| 1096 | ++ |
| 1098 | ++ |
| 1099 | ++ |
| 1102 | +++ |
| 1103 | +++ |
| 1104 | ++ |
| 1105 | +++ |
| 1106 | +++ |
| 1107 | +++ |
| 1108 | +++ |
| 1109 | ++ |
| 1110 | +++ |
| 1111 | +++ |
| 1112 | ++ |
| 1113 | ++ |
| 1115 | +++ |
| 1116 | ++ |
| 1117 | ++ |
| 1119 | +++ |
| 1121 | +++ |
| 1122 | ++ |
| 1123 | +++ |
| 1124 | +++ |
| 1125 | +++ |
| 1126 | +++ |
| 1127 | +++ |
| 1128 | +++ |
| 1129 | +++ |
| 1130 | +++ |
| 1131 | ++ |
| 1132 | +++ |
| 1133 | ++ |
| 1134 | +++ |
| 1136 | ++ |
| 1137 | ++ |

Activity is provided as follows:
+++ = IC₅₀ < 1,000 nM
++ = 1,000 nM < IC₅₀ < 10,000 nM

### Degradation Assay: Screening Compounds Procedure to Evaluate the Activity of Compounds in Degrading Endogenous MLKL in Human and Mouse Cell

The mouse dermal fibroblast (MDF) cell line was seeded the day before the experiment in a 24-well plate at a cell density of 5*10∝cells per well in a volume of 0.5 mL per well and left to attach overnight. The human monocyte cell line (U937) was seeded on the day of the experiment in a 24-well plate at a cell density of 5*10≡cells per well in a volume of 0.5 mL per well.

The compound was diluted in DMSO with a starting concentration of 10 mM and titrated stepwise with a dilution of 1:5. 0.5 μL of the compound dilution was added to the appropriate well to achieve a final concentration of 10; 2; 0.4; 0.08 and 0.016 μM. A DMSO only well was included as control.

The cells were incubated for 24 h in the presence of the compound at 37° C. and 10% CO₂.

After the 24 h incubation period, the medium was removed and cells washed with 0.5 mL PBS. The cells were then lysed directly in 60 μL of 1×SDS gel loading buffer.

The cell lysate was heat-denatured at 1000 for 10 min. 10 μL of lysate per well were run on 18-well 4-15% Criterion TGX stain free gels (BIO-RAD). The proteins were then transferred onto nitrocellulose membrane with a wet transfer protocol in the Criterion Blotter (BIO-RAD).

Membranes were blocked with 5% skim milk in TBST. Primary antibody against MLKL (clone 3H1, MABC604 Merck) was used at 1:2000 dilution in TBST with 1% BSA and left shaking over night at 4° C. After washes with TBST, the membranes were incubated with goat anti-rat IgG HRP-conjugated antibody (Southern Biotech) in 1% skim milk TBST at 1:10000 dilution for 1 h at room temperature. After TBST washes the bands were imaged on the ChemiDoc MP Imaging System (BIO-RAD) with Immobilion Western Chemiluminescent HRP Substrate Forte (Millipore). The loading control GAPDH was probed for with rabbit anti-GAPDH (CST 14C10) at 1:2000 dilution in 1% BSA TBST over night at 4° C. TBST washes were followed by incubation with goat anti-rabbit IgG HRP-conjugated antibody (Southern Biotech) in 1% skim milk TBST at 1:10000 dilution for 1 h at room temperature followed by TBST washes. The signal was detected as described above.

The band intensity was measured in the ImageLab Software (BIO-RAD) and the MLKL values adjusted for loading with GAPDH. The percentage of degradation was calculated by normalizing the compound treated values to the DMSO only control.

TABLE 28
Table showing degradation of endogenous
MLKL in U937 (DC₅₀ cell-based assay)

| Compound | Degradation of endogenous MLKL in |
|---|---|
| No | U937 (DC₅₀ cell-based assay) |
| 1003 | ** |
| 1005 | ** |
| 1006 | *** |
| 1008 | *** |
| 1009 | ** |
| 1010 | ** |
| 1011 | ** |
| 1012 | ** |
| 1017 | ** |
| 1014 | *** |
| 1015 | ** |
| 1022 | *** |
| 1040 | ** |
| 1041 | ** |
| 1042 | *** |
| 1044 | *** |
| 1046 | *** |
| 1047 | *** |
| 1048 | ** |
| 1050 | *** |
| 1051 | ** |
| 1052 | ** |
| 1054 | *** |
| 1056 | *** |
| 1057 | *** |
| 1058 | ** |
| 1059 | *** |
| 1068 | ** |
| 1069 | *** |
| 1072 | *** |
| 1073 | *** |
| 1074 | *** |
| 1075 | *** |
| 1076 | *** |
| 1077 | ** |
| 1078 | ** |
| 1079 | *** |
| 1081 | *** |
| 1082 | *** |
| 1083 | *** |
| 1085 | *** |
| 1088 | *** |
| 1091 | *** |
| 1093 | *** |
| 1095 | *** |
| 1097 | *** |
| 1098 | *** |
| 1099 | *** |
| 1101 | *** |
| 1104 | *** |
| 1108 | ** |
| 1109 | *** |
| 1113 | *** |
| 1114 | *** |
| 1115 | *** |
| 1120 | |
| 1121 | *** |
| 1122 | *** |
| 1123 | *** |
| 1124 | *** |
| 1125 | *** |
| 1126 | *** |
| 1127 | *** |
| 1129 | *** |
| 1130 | *** |
| 1131 | *** |
| 1134 | *** |

*** = DC₅₀ < 1 μM
** = 1 uM < DC₅₀ < 10 μM
* = DC₅₀ > 10 μM

TABLE 29
Table showing degradation of endogenous
MLKL in MDF (DC₅₀ cell-based assay)

| Compound | Degradation of endogenous MLKL in |
|---|---|
| No | MDF (DC₅₀ cell-based assay) |
| 1022 | *** |
| 1023 | ** |
| 1031 | *** |
| 1027 | ** |
| 1028 | ** |
| 1039 | ** |
| 1041 | *** |
| 1042 | *** |
| 1043 | *** |
| 1044 | *** |
| 1045 | *** |
| 1047 | *** |
| 1048 | *** |
| 1049 | *** |
| 1050 | ** |
| 1051 | *** |
| 1053 | *** |
| 1054 | *** |
| 1055 | *** |
| 1056 | ** |
| 1057 | ** |
| 1058 | *** |
| 1059 | *** |
| 1060 | ** |
| 1068 | *** |
| 1069 | *** |
| 1070 | *** |
| 1071 | *** |
| 1072 | *** |
| 1073 | *** |
| 1074 | *** |
| 1076 | ** |
| 1078 | ** |
| 1079 | *** |
| 1081 | *** |
| 1082 | ** |
| 1083 | *** |
| 1085 | ** |
| 1087 | *** |
| 1088 | *** |
| 1090 | ** |
| 1091 | *** |
| 1093 | *** |
| 1094 | *** |
| 1095 | *** |
| 1096 | *** |
| 1098 | *** |
| 1099 | ** |
| 1100 | *** |
| 1102 | *** |
| 1103 | *** |
| 1104 | *** |
| 1105 | *** |
| 1106 | *** |
| 1107 | *** |
| 1108 | *** |
| 1109 | *** |
| 1110 | *** |
| 1111 | *** |
| 1113 | *** |
| 1114 | *** |
| 1115 | *** |
| 1116 | *** |
| 1118 | *** |
| 1119 | *** |
| 1121 | *** |
| 1122 | ** |
| 1123 | *** |
| 1124 | *** |
| 1125 | *** |
| 1126 | *** |
| 1127 | *** |
| 1128 | *** |
| 1129 | *** |
| 1130 | *** |
| 1131 | *** |
| 1132 | *** |
| 1133 | *** |
| 1134 | *** |
| 1135 | ** |
| 1137 | *** |

*** = DC₅₀ < 1 μM
** = 1 uM < DC₅₀ < 10 μM

It will be appreciated by persons skilled in the art that numerous variations and/or modifications may be made to the above-described embodiments, without departing from the broad general spirit and scope of the present disclosure. The present embodiments are, therefore, to be considered in all respects as illustrative and not restrictive.

1. A compound of formula (X):
MLKLi-L-E3L (X)
wherein
E3L is an E3 ligase binding moiety;
L is a linker covalently linking MLKLi to E3L; and
MLKLi is a radical of formula (I): 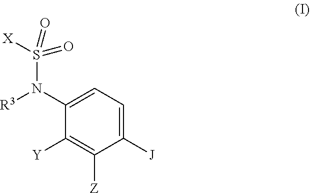
wherein
J is selected from the following moieties J1 and J2: 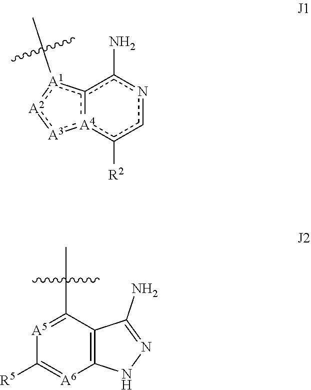
A¹ and A⁴ are independently selected from N and C;
A² and A³ are independently selected from N, NR¹, CH, O and S;
wherein at least one of A¹, A², A³ and A⁴ is selected from N, NR¹, O and S;
A⁵ is CH or N;
A⁶ is N or CR²;
R¹ and R³ are independently selected from H and an optionally substituted C₁₋₆-alkyl;
R² is selected from:
(i) H,
(ii) optionally substituted C₁₋₄alkylamido,
(iii) optionally substituted C₁₋₄alkylaryl,
(iv) optionally substituted C₂₋₄alkynyl,
(v) optionally substituted aryl,
(vi) optionally substituted 5- or 6-membered heterocyclyl,
(vii) cyano;
X is selected from optionally substituted C₁₋₆alkyl, optionally substituted haloC₁₋₆alkyl, optionally substituted C₂₋₆alkynyl, optionally substituted cycloalkyl, optionally substituted halocycloalkyl, optionally substituted aryl, optionally substituted alkylaryl, optionally substituted C₁₋₆alkylcycloalkyl and optionally substituted amino;
Y and Z are independently selected from H, R⁴, —OR⁴, —NR⁴R^{4′}, and halo,
wherein at least one of Y and Z is H;
R⁴ is selected from optionally substituted C₁₋₆alkyl, optionally substituted aryl, optionally substituted C₁₋₆alkylaryl, optionally substituted heterocyclyl, optionally substituted C₁₋₆alkylheterocyclyl, optionally substituted cycloalkyl, optionally substituted C₁₋₆alkylC₃₋₁₀cycloalkyl, optionally substituted C₃₋₁₀cycloalkylaryl, optionally substituted C₃₋₁₀cycloalkylheterocyclyl, optionally substituted C₃₋₁₀cycloalkylC₃₋₁₀cycloalkyl, optionally substituted 3-6 membered non-aromatic heterocyclyl-aryl, optionally substituted 3-6 membered non-aromatic heterocyclylC₃₋₁₀cycloalkyl and optionally substituted 3-6 membered non-aromatic heterocyclyl-3-10 membered heterocyclyl;
R^{4′} is H or optionally substituted C₁₋₆alkyl, and
R⁵ is selected from H, optionally substituted C₁₋₆alkyl, optionally substituted C₃₋₁₀cycloalkyl and optionally substituted aryl;
or a pharmaceutically acceptable salt, solvate, tautomer, N-oxide, stereoisomer and/or prodrug thereof.

2. The compound of claim 1, or a pharmaceutically acceptable salt, solvate, tautomer, N-oxide, stereoisomer and/or prodrug thereof, wherein J is J1.

3. The compound of claim 1 or 2, or a pharmaceutically acceptable salt, solvate, tautomer, N-oxide, stereoisomer and/or prodrug thereof, wherein the compound of formula (X) is provided as a compound of formula (XX):

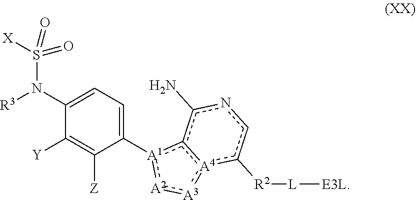

4. The compound of claim 3, or a pharmaceutically acceptable salt, solvate, tautomer, N-oxide, stereoisomer and/or prodrug thereof, wherein R² is selected from:
(vi) C₁₋₄alkylamido,
(vii) C₁₋₄alkylaryl,
(viii) C₂₋₄alkynyl,
(ix) aryl,
(x) 5- or 6-membered heterocyclyl
wherein each of groups (i)-(v) is optionally substituted with 1-3 groups independently selected from: halo, an optionally substituted C₁₋₆alkyl, an optionally substituted C₃₋₈cycloalkyl, an optionally substituted aryl, an optionally substituted heterocyclyl, an optionally substituted C₁₋₄alkoxy, an optionally substituted C₁₋₄alkyl-OH, an optionally substituted C₁₋₄alkylhalo, an optionally substituted C₁₋₄alkylheterocyclyl, an optionally substituted C₁₋₄alkylC₃₋₈cycloalkyl, an optionally substituted C₁₋₄alkylaryl.

5. The compound of any one of claims 1-4, or a pharmaceutically acceptable salt, solvate, tautomer, N-oxide, stereoisomer and/or prodrug thereof, wherein the E3 ligase binding moiety is selected from 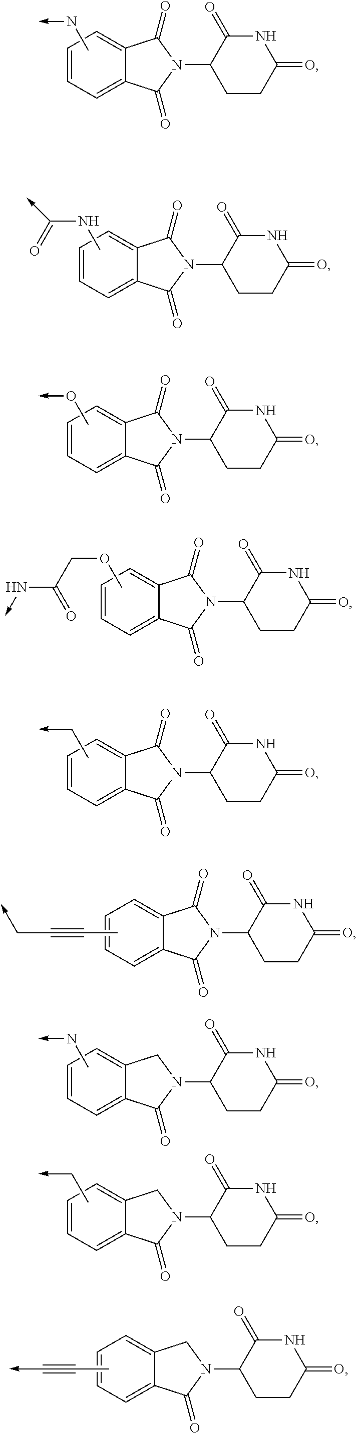 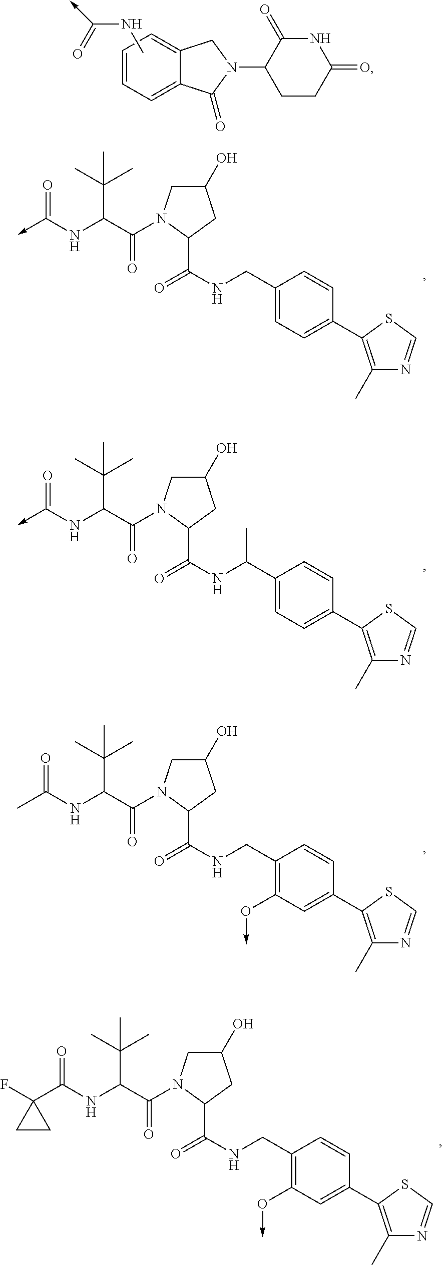 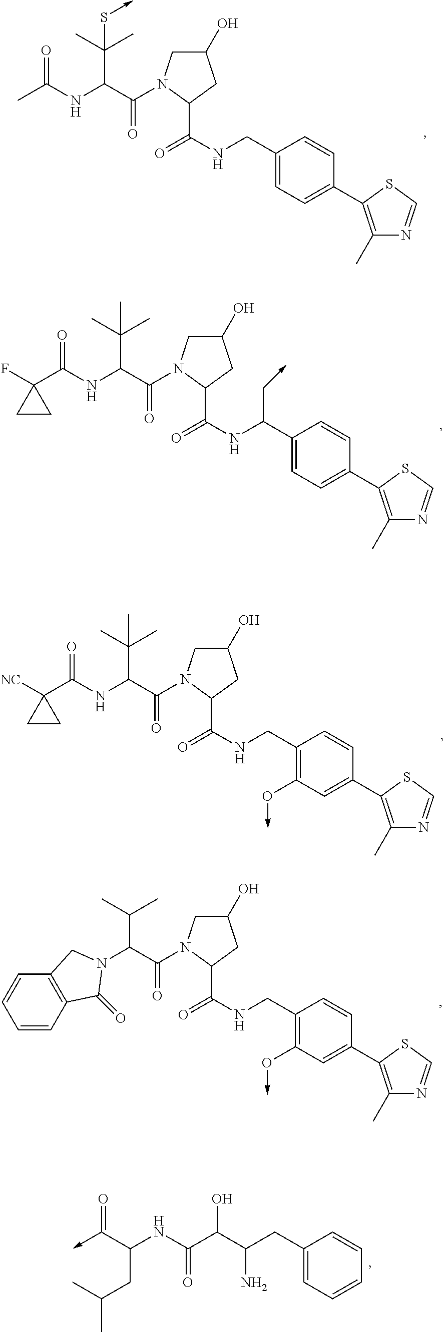 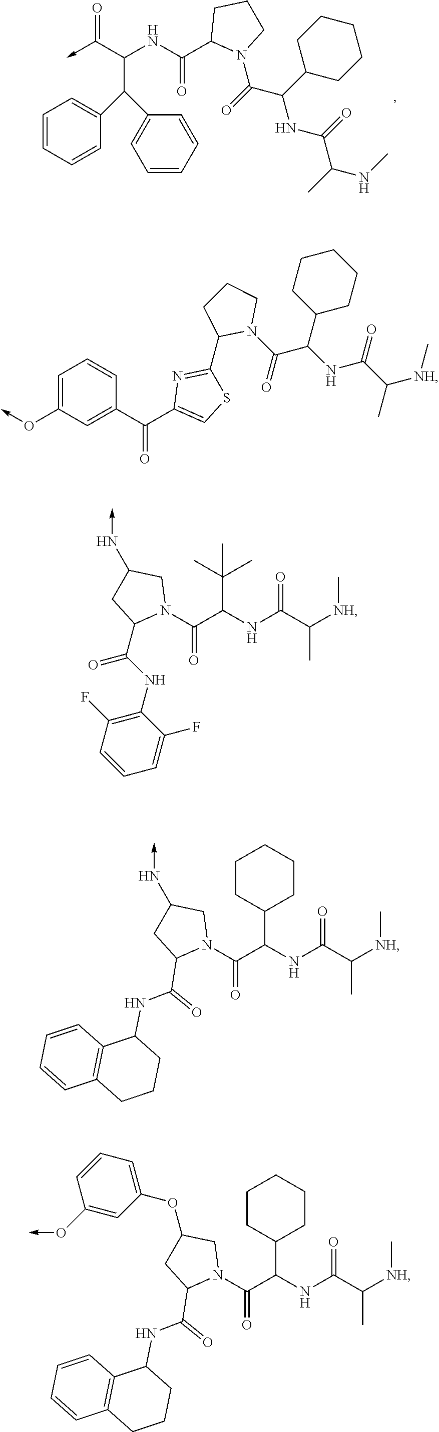 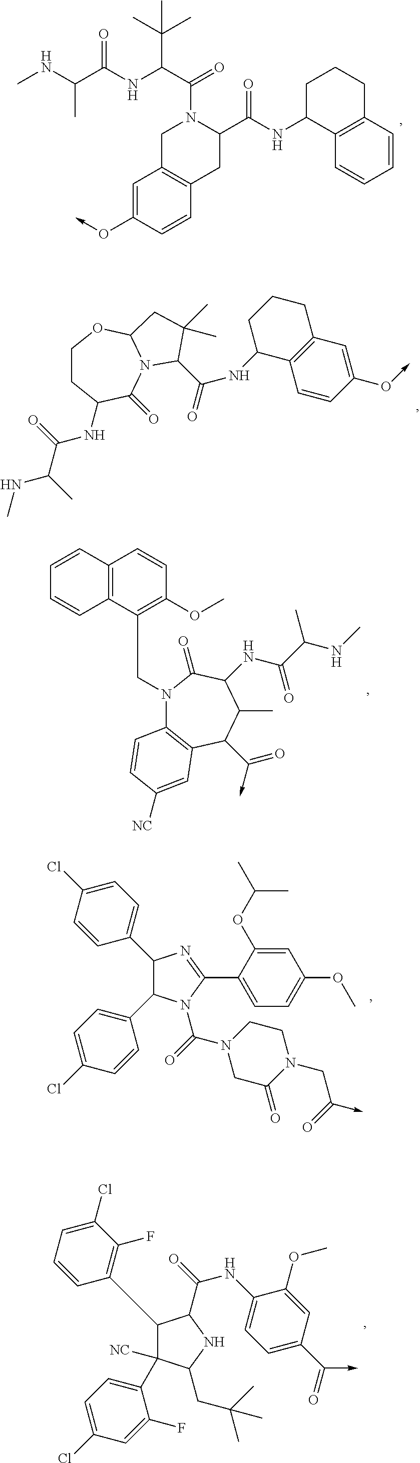 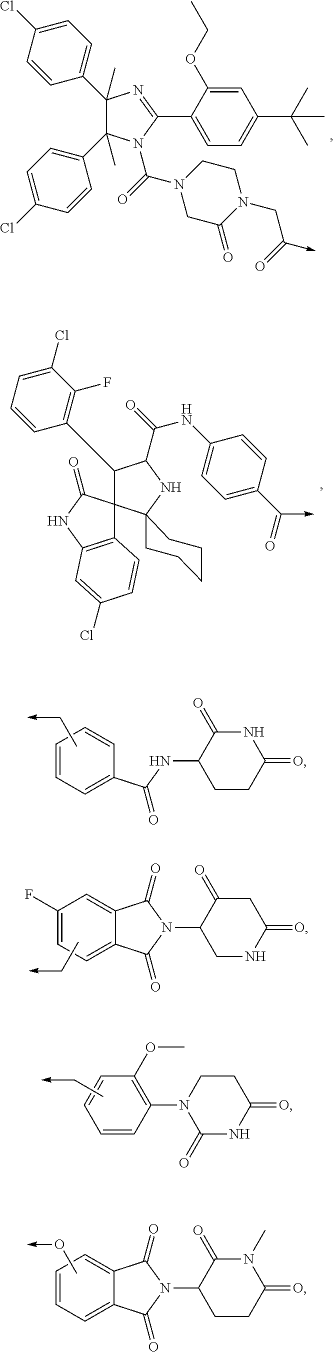 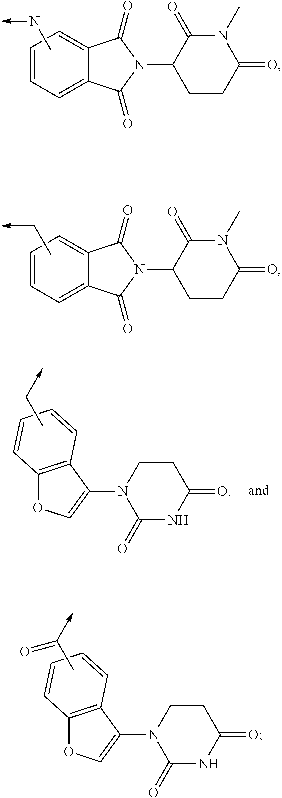
wherein the arrow denotes the covalent bond to L,
the portion of L not depicted in the structure, or
an E3 ligase binding derivative thereof.

6. The compound of any one of claims 1-5, or a pharmaceutically acceptable salt, solvate, tautomer, N-oxide, stereoisomer and/or prodrug thereof, wherein the E3 ligase binding moiety is selected from 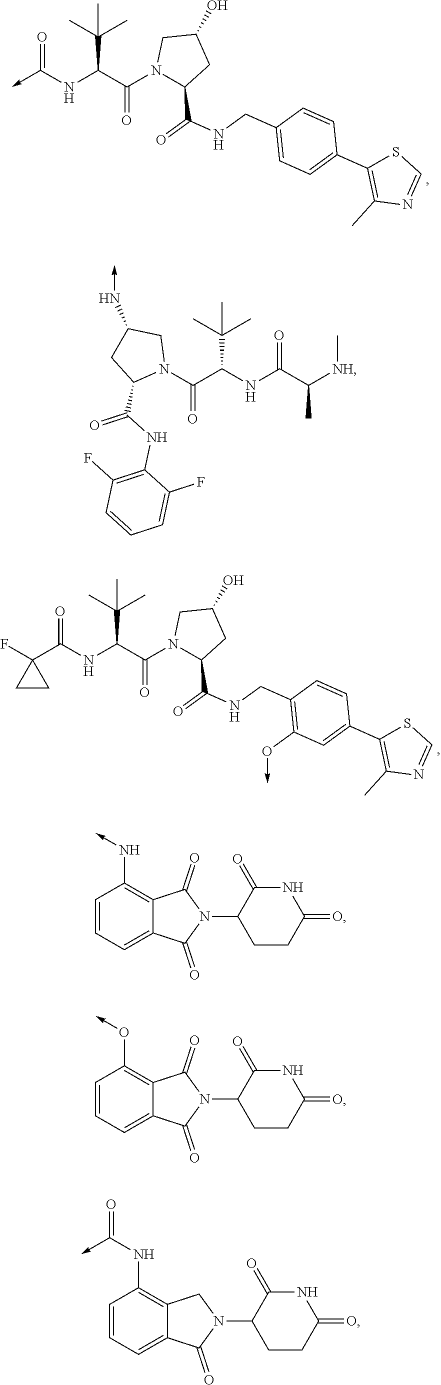 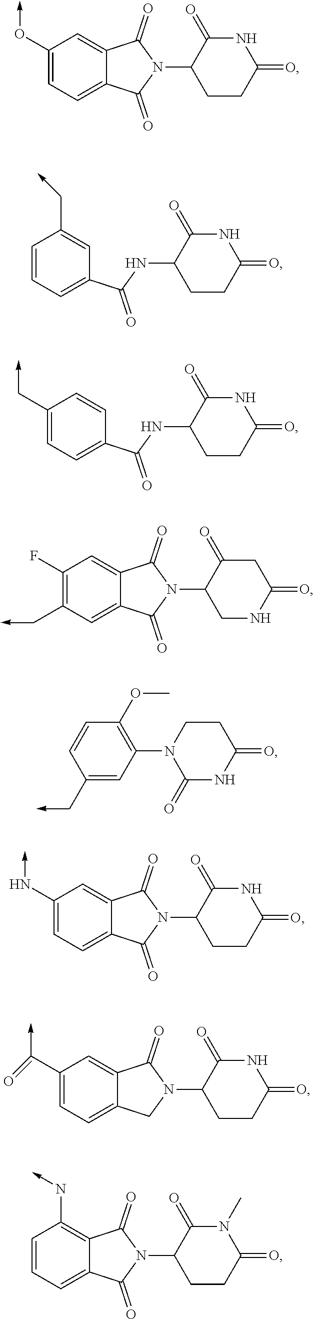 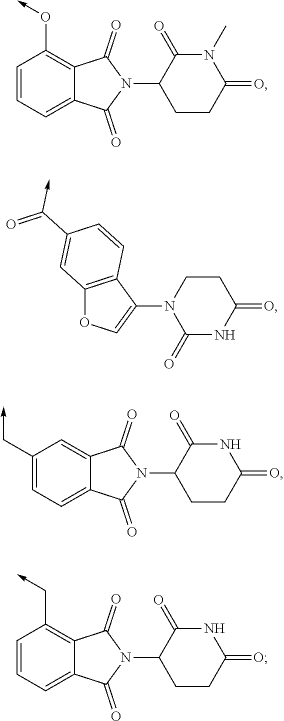
wherein the arrow denotes the covalent bond to L,
the portion of L not depicted in the structure, or
an E3 ligase binding derivative thereof.

7. The compound of any one of claims 1-6, or a pharmaceutically acceptable salt, solvate, tautomer, N-oxide, stereoisomer and/or prodrug thereof, wherein the linker has a shortest linear chain length of 1 to 50 atoms.

8. The compound of claim 7, or a pharmaceutically acceptable salt, solvate, tautomer, N-oxide, stereoisomer and/or prodrug thereof, wherein the linker has a shortest linear chain length of 1 to 10 atoms.

9. The compound of any one of claims 1-8, or a pharmaceutically acceptable salt, solvate, tautomer, N-oxide, stereoisomer and/or prodrug thereof, wherein the linker is a C₁₋₅₀alkyl optionally interrupted by one or more groups selected from:
a. —O—,
b. —NR^{z}—,
c. C₃₋₈cycloalkyl,
d. aryl,
e. C₁₋₄alkaryl,
f. heteroaryl,
g. (C₁₋₄alkoxy)₁₋₄aryl,
h. haloaryl,
i. 4-8-membered non-aromatic heterocyclyl,
j. —C(O)NR^{z}—, wherein each R^{Z} is independently selected from H and C₁₋₄alkyl,
k. alkenyl,
l. alkynyl,
and wherein each of the one or more groups a-I may be further optionally substituted with a group selected from: C₃₋₆cycloalkyl, halo, —OH, —CN, —NR^{z2}, C₁₋₄alkyl, C₁₋₄alkoxy, oxo, C₁₋₄ alkylketone, —COOH, —C(O)N(R^{z})₂, and —NR^{z}C(O)R^{z}.

10. The compound of claim 9, or a pharmaceutically acceptable salt, solvate, tautomer, N-oxide, stereoisomer and/or prodrug thereof, wherein the C₁₋₅₀alkyl is optionally interrupted by 1-20 of the one or more groups.

11. The compound of claim 9 or 10, or a pharmaceutically acceptable salt, solvate, tautomer, N-oxide, stereoisomer and/or prodrug thereof, wherein the linker comprises the moiety —(OCH₂CH₂)ᵥ—, wherein v is an integer from 1 to 15.

12. The compound of any one of claims 1-11, or a pharmaceutically acceptable salt, solvate, tautomer, N-oxide, stereoisomer and/or prodrug thereof, wherein the linker comprises at least one coupling moiety selected from: —C(O)O—, —C(O)NR^{z}—, —OC(O)O—, —NR^{z}C(O)NR^{z}—, —OC(O)NR^{z}—, triazolyl, aryl, α,μ-unsubstituted ketone, p-hydroxy-ketone, 4-8-membered heteroaryl, unsaturated C₆-cycloalkyl and optionally substituted C₂alkenyl, wherein each R^{z} is independently selected from H and C₁₋₄alkyl.

13. The compound of any one of claims 1-12 selected from any of the following compounds: 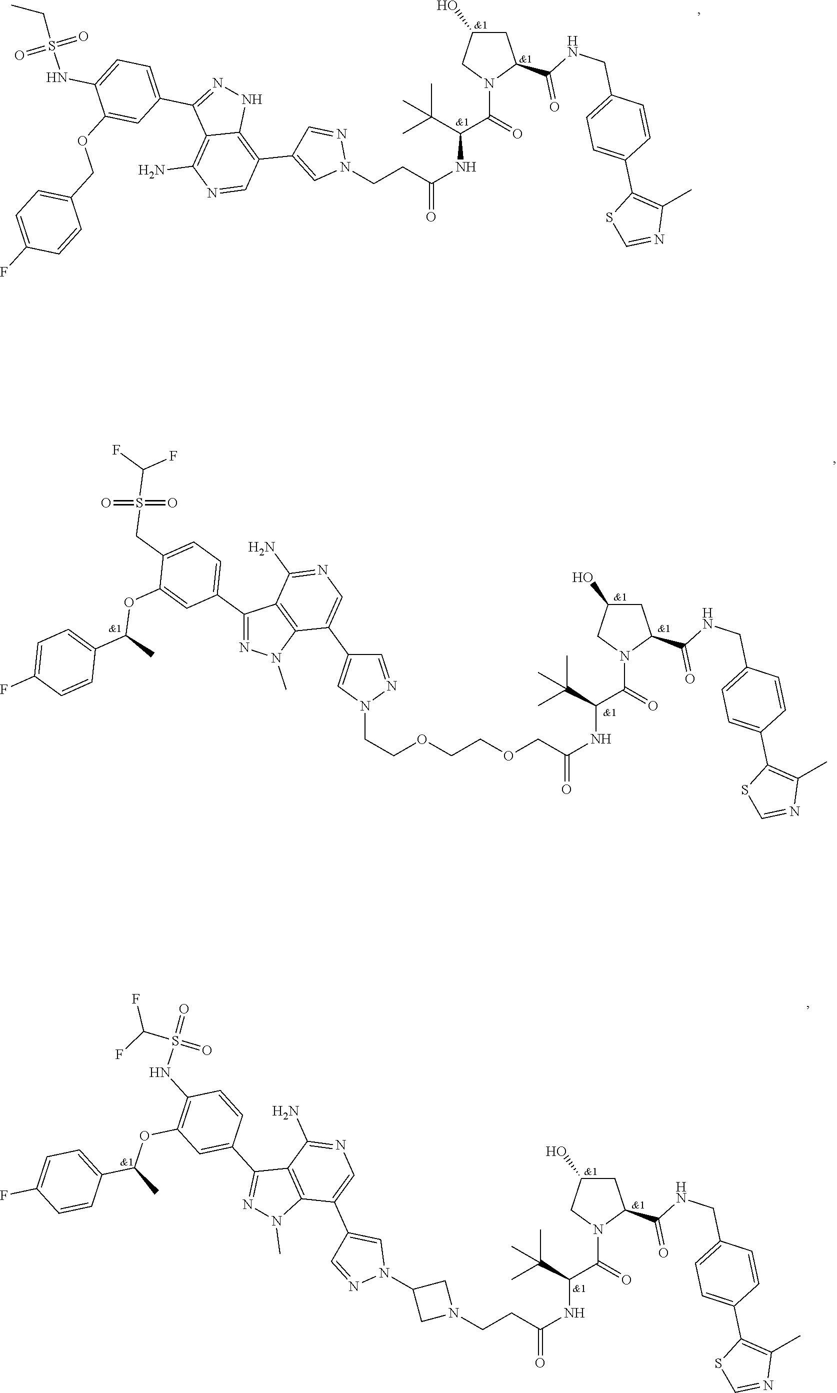 [SMILES: CCS(=O)(=O)Nc1ccc(-c2n[nH]c3c(-c4cnn(CCC(=O)N[C@H](C(=O)N5C[C@H](O)C[C@H]5C(=O)NCc5ccc(-c6scnc6C)cc5)C(C)(C)C)c4)cnc(N)c23)cc1OCc1ccc(F)cc1 ; Cc1ncsc1-c1ccc(CNC(=O)[C@@H]2C[C@H](O)CN2C(=O)[C@@H](NC(=O)COCCOCCn2cc(-c3cnc(N)c4c(-c5ccc(CS(=O)(=O)C(F)F)c(O[C@@H](C)c6ccc(F)cc6)c5)nn(C)c34)cn2)C(C)(C)C)cc1 ; Cc1ncsc1-c1ccc(CNC(=O)[C@@H]2C[C@@H](O)CN2C(=O)[C@@H](NC(=O)CCN2CC(n3cc(-c4cnc(N)c5c(-c6ccc(NS(=O)(=O)C(F)F)c(O[C@@H](C)c7ccc(F)cc7)c6)nn(C)c45)cn3)C2)C(C)(C)C)cc1] 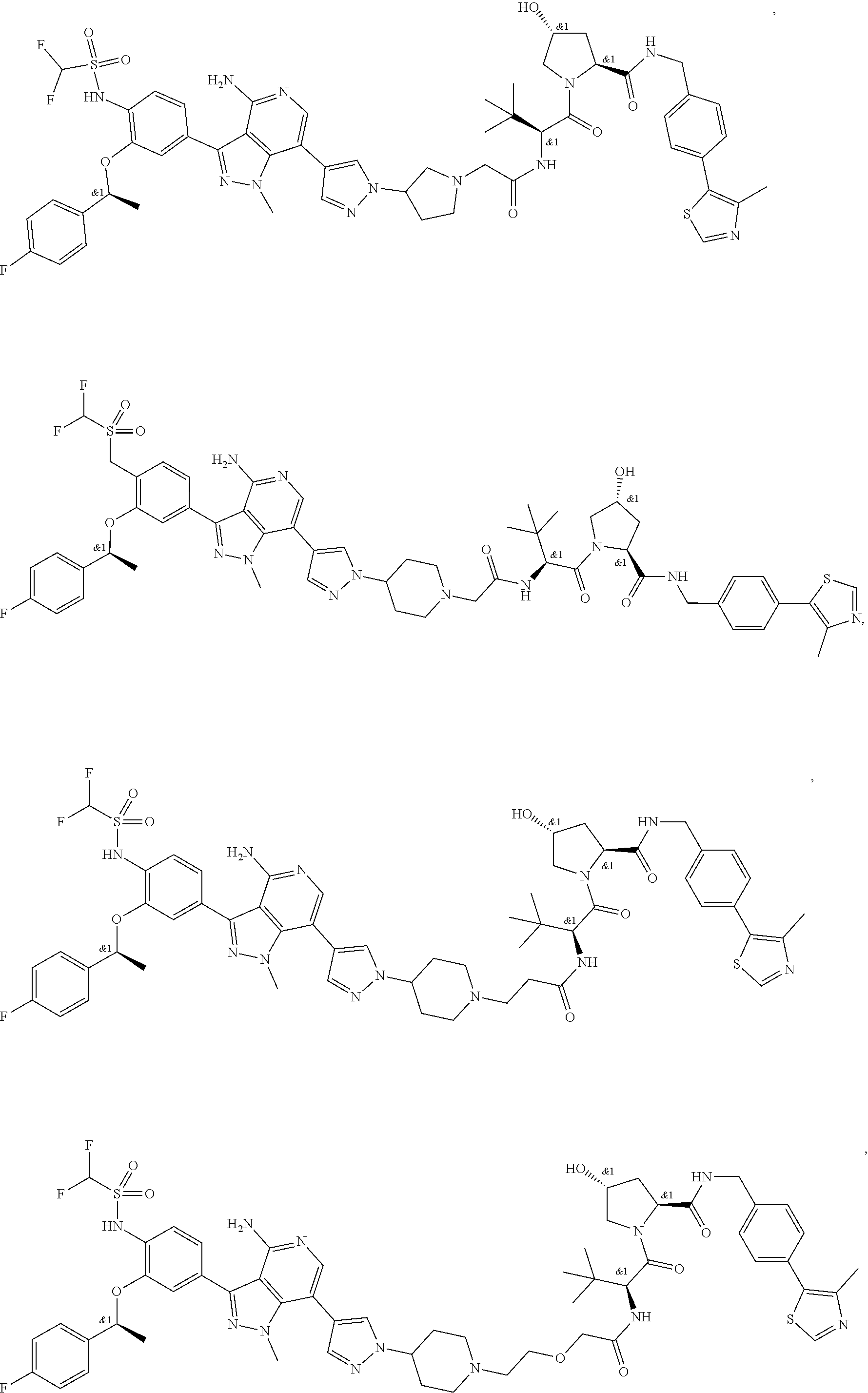 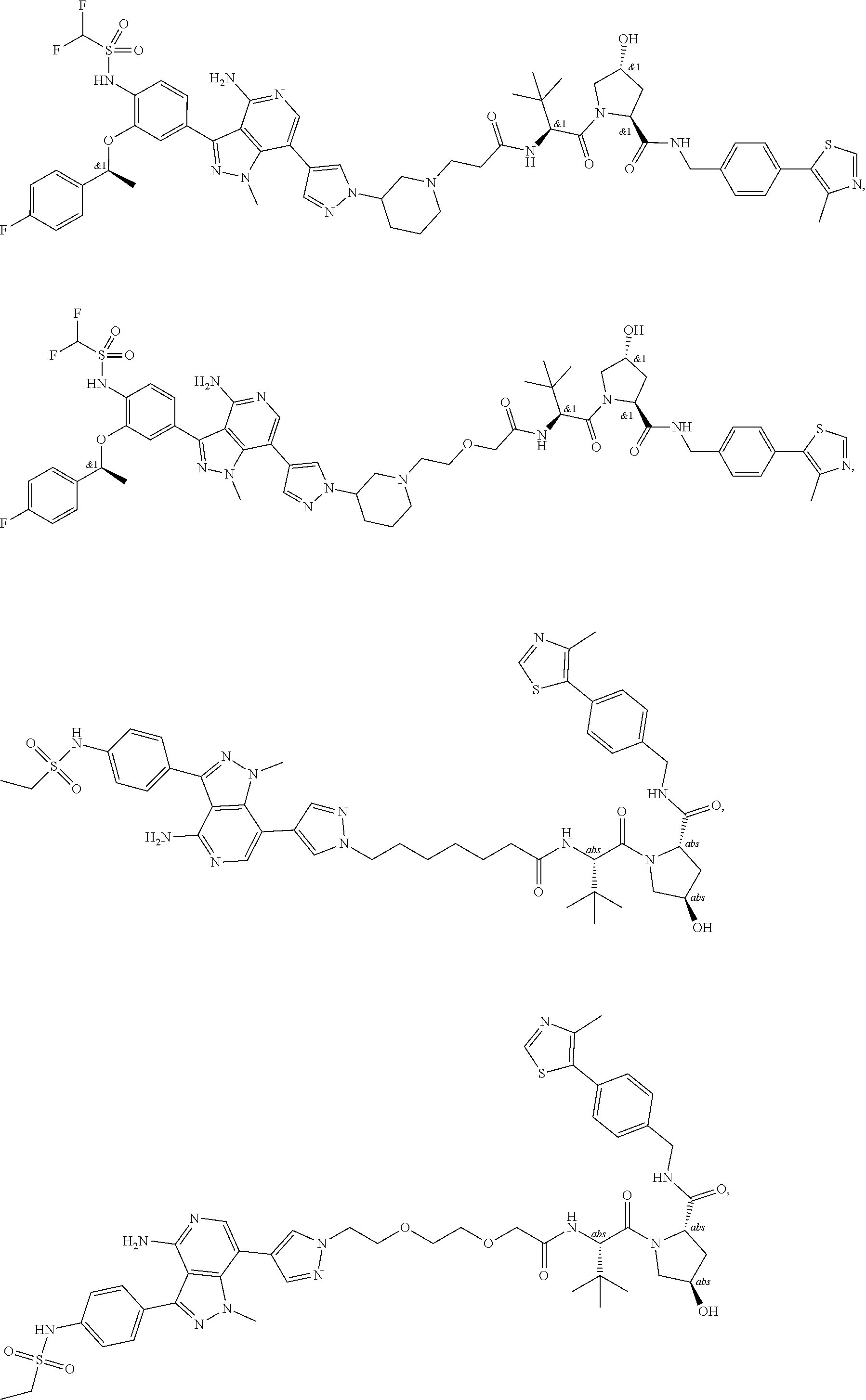 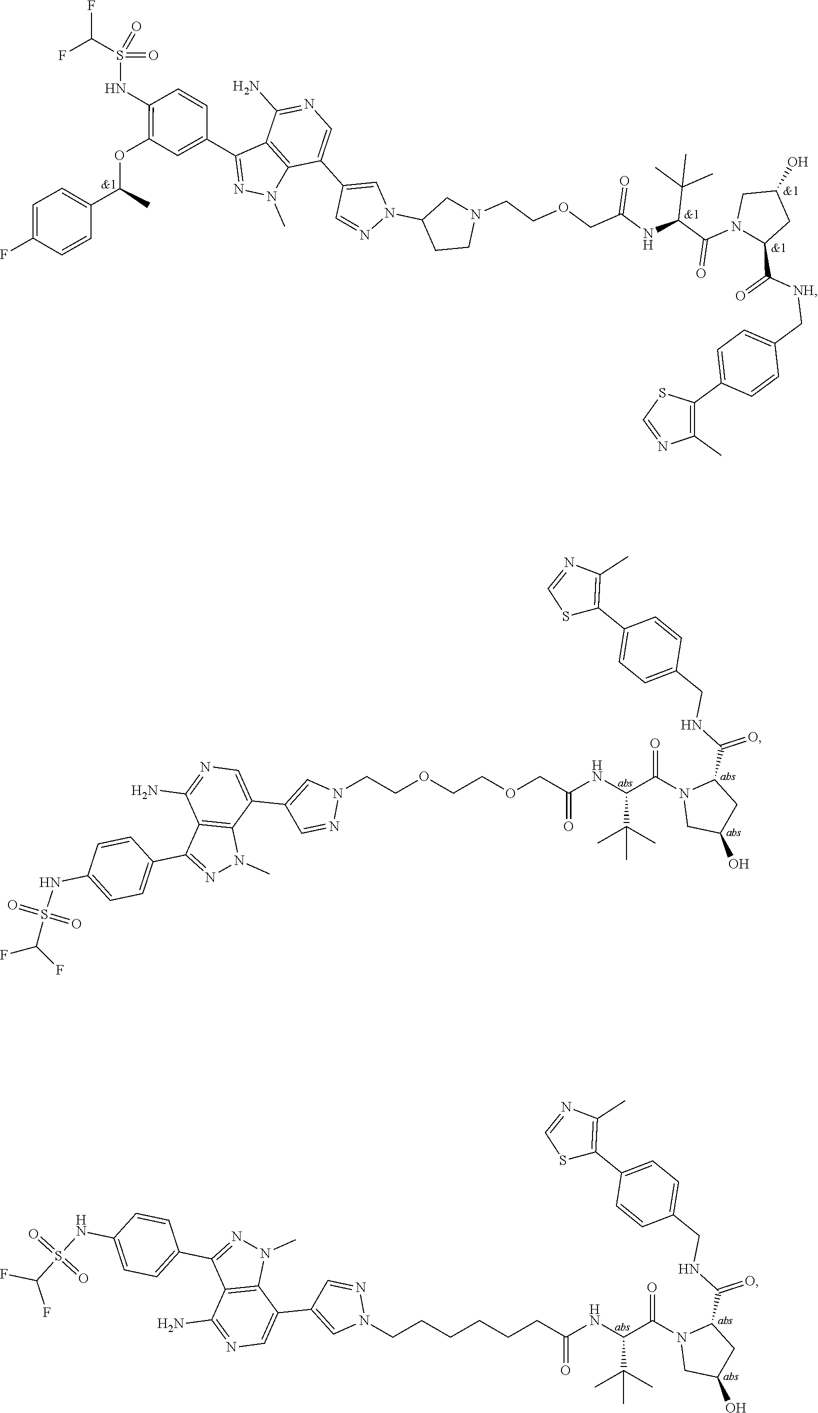 [SMILES: Cc1ncsc1-c1ccc(CNC(=O)[C@@H]2C[C@@H](O)CN2C(=O)[C@@H](NC(=O)COCCN2CCC(n3cc(-c4cnc(N)c5c(-c6ccc(NS(=O)(=O)C(F)F)c(O[C@@H](C)c7ccc(F)cc7)c6)nn(C)c45)cn3)C2)C(C)(C)C)cc1 ; Cc1ncsc1-c1ccc(CNC(=O)[C@@H]2C[C@@H](O)CN2C(=O)[C@@H](NC(=O)COCCOCCn2cc(-c3cnc(N)c4c(-c5ccc(NS(=O)(=O)C(F)F)cc5)nn(C)c34)cn2)C(C)(C)C)cc1 ; Cc1ncsc1-c1ccc(CNC(=O)[C@@H]2C[C@@H](O)CN2C(=O)[C@@H](NC(=O)CCCCCCn2cc(-c3cnc(N)c4c(-c5ccc(NS(=O)(=O)C(F)F)cc5)nn(C)c34)cn2)C(C)(C)C)cc1] 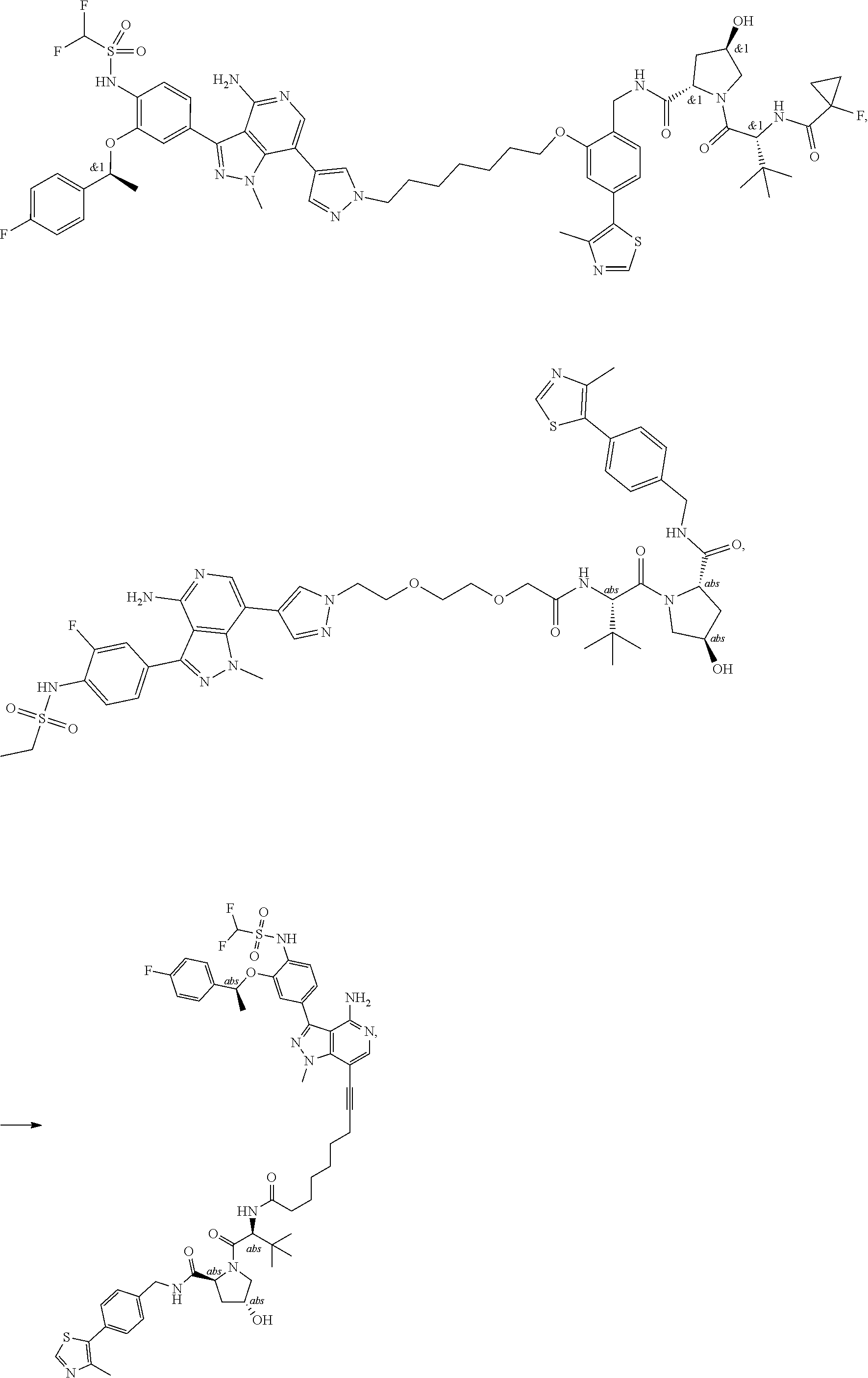 [SMILES: Cc1ncsc1-c1ccc(CNC(=O)[C@@H]2C[C@@H](O)CN2C(=O)[C@H](NC(=O)C2(F)CC2)C(C)(C)C)c(OCCCCCCCn2cc(-c3cnc(N)c4c(-c5ccc(NS(=O)(=O)C(F)F)c(O[C@@H](C)c6ccc(F)cc6)c5)nn(C)c34)cn2)c1 ; CCS(=O)(=O)Nc1ccc(-c2nn(C)c3c(-c4cnn(CCOCCOCC(=O)N[C@H](C(=O)N5C[C@H](O)C[C@H]5C(=O)NCc5ccc(-c6scnc6C)cc5)C(C)(C)C)c4)cnc(N)c23)cc1F ; Cc1ncsc1-c1ccc(CNC(=O)[C@@H]2C[C@@H](O)CN2C(=O)[C@@H](NC(=O)CCCCCCC#Cc2cnc(N)c3c(-c4ccc(NS(=O)(=O)C(F)F)c(O[C@@H](C)c5ccc(F)cc5)c4)nn(C)c23)C(C)(C)C)cc1] 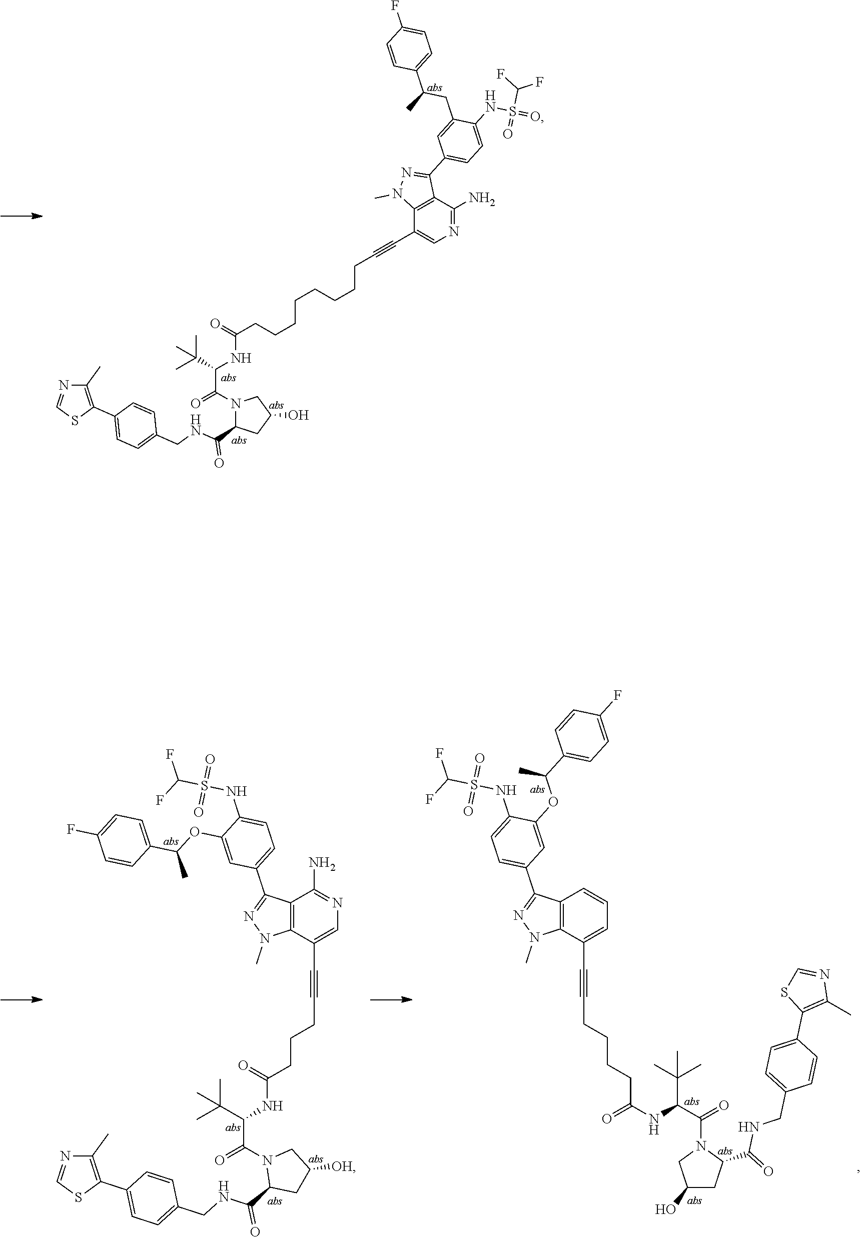 [SMILES: Cc1ncsc1-c1ccc(CNC(=O)[C@@H]2C[C@@H](O)CN2C(=O)[C@@H](NC(=O)CCCCCCCCC#Cc2cnc(N)c3c(-c4ccc(NS(=O)(=O)C(F)F)c(C[C@@H](C)c5ccc(F)cc5)c4)nn(C)c23)C(C)(C)C)cc1 ; Cc1ncsc1-c1ccc(CNC(=O)[C@@H]2C[C@@H](O)CN2C(=O)[C@@H](NC(=O)CCCC#Cc2cnc(N)c3c(-c4ccc(NS(=O)(=O)C(F)F)c(O[C@@H](C)c5ccc(F)cc5)c4)nn(C)c23)C(C)(C)C)cc1 ; Cc1ncsc1-c1ccc(CNC(=O)[C@@H]2C[C@@H](O)CN2C(=O)[C@@H](NC(=O)CCCCC#Cc2cccc3c(-c4ccc(NS(=O)(=O)C(F)F)c(O[C@@H](C)c5ccc(F)cc5)c4)nn(C)c23)C(C)(C)C)cc1] 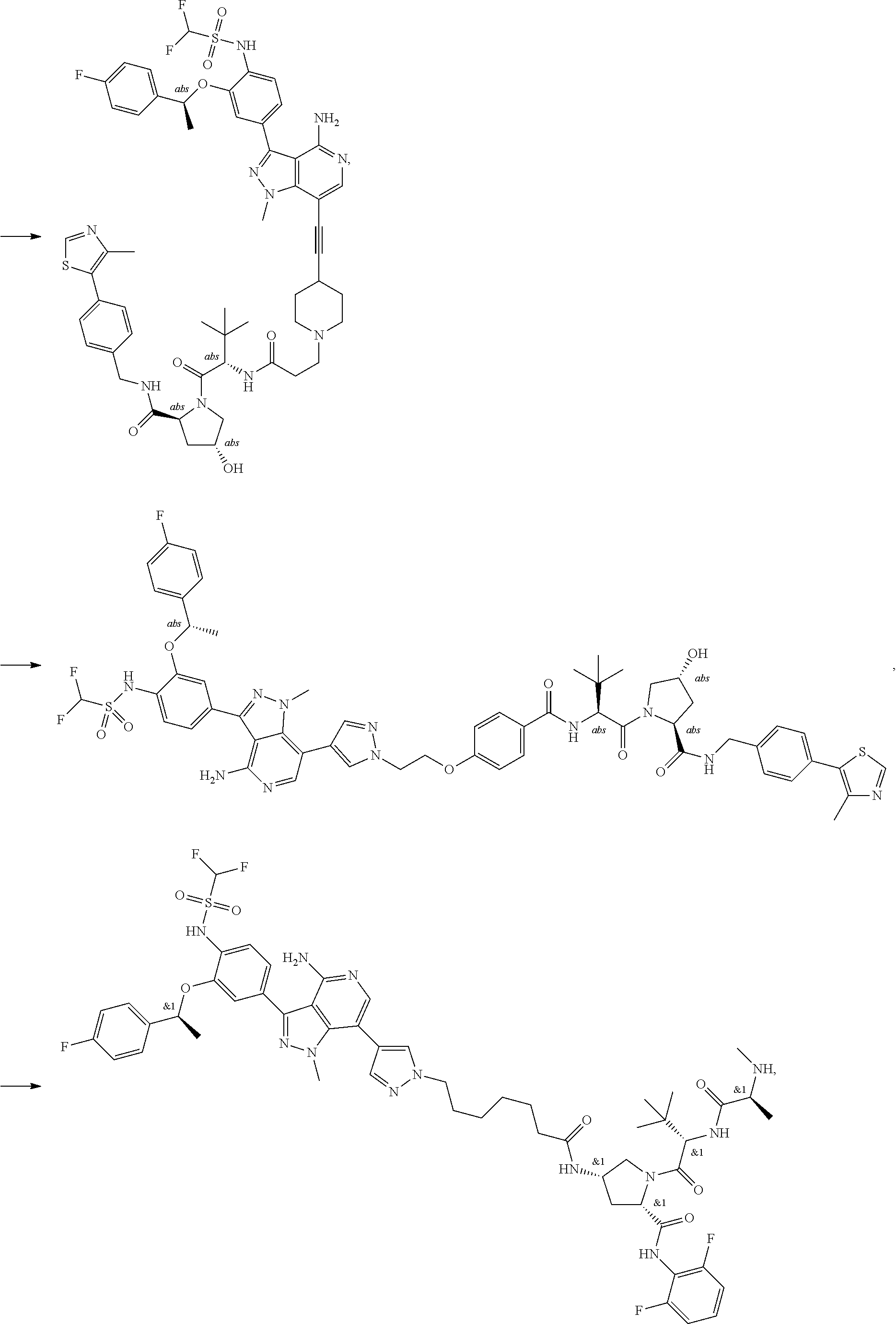 [SMILES: Cc1ncsc1-c1ccc(CNC(=O)[C@@H]2C[C@@H](O)CN2C(=O)[C@@H](NC(=O)CCN2CCC(C#Cc3cnc(N)c4c(-c5ccc(NS(=O)(=O)C(F)F)c(O[C@@H](C)c6ccc(F)cc6)c5)nn(C)c34)CC2)C(C)(C)C)cc1 ; Cc1ncsc1-c1ccc(CNC(=O)[C@@H]2C[C@@H](O)CN2C(=O)[C@@H](NC(=O)c2ccc(OCCn3cc(-c4cnc(N)c5c(-c6ccc(NS(=O)(=O)C(F)F)c(O[C@@H](C)c7ccc(F)cc7)c6)nn(C)c45)cn3)cc2)C(C)(C)C)cc1 ; CN[C@@H](C)C(=O)N[C@H](C(=O)N1C[C@@H](NC(=O)CCCCCCn2cc(-c3cnc(N)c4c(-c5ccc(NS(=O)(=O)C(F)F)c(O[C@@H](C)c6ccc(F)cc6)c5)nn(C)c34)cn2)C[C@H]1C(=O)Nc1c(F)cccc1F)C(C)(C)C] 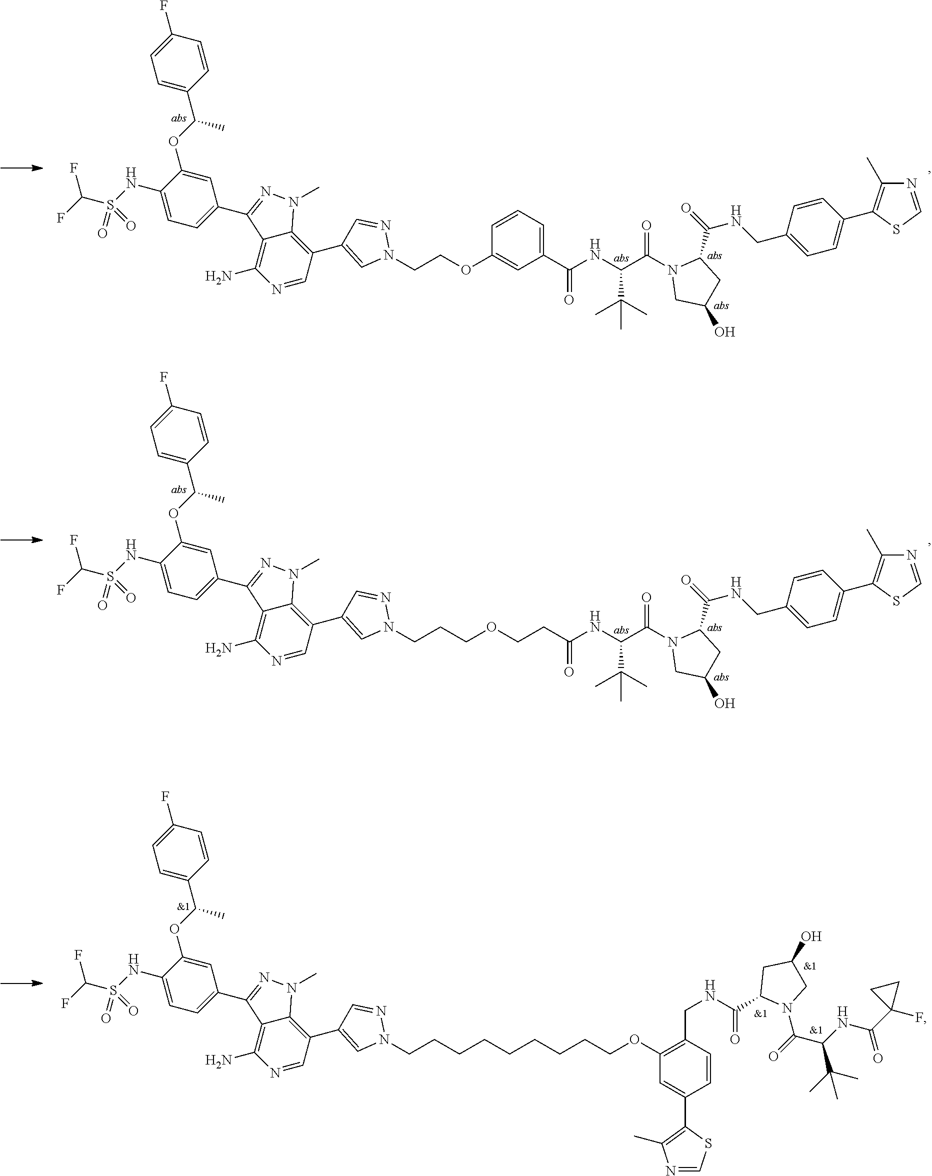 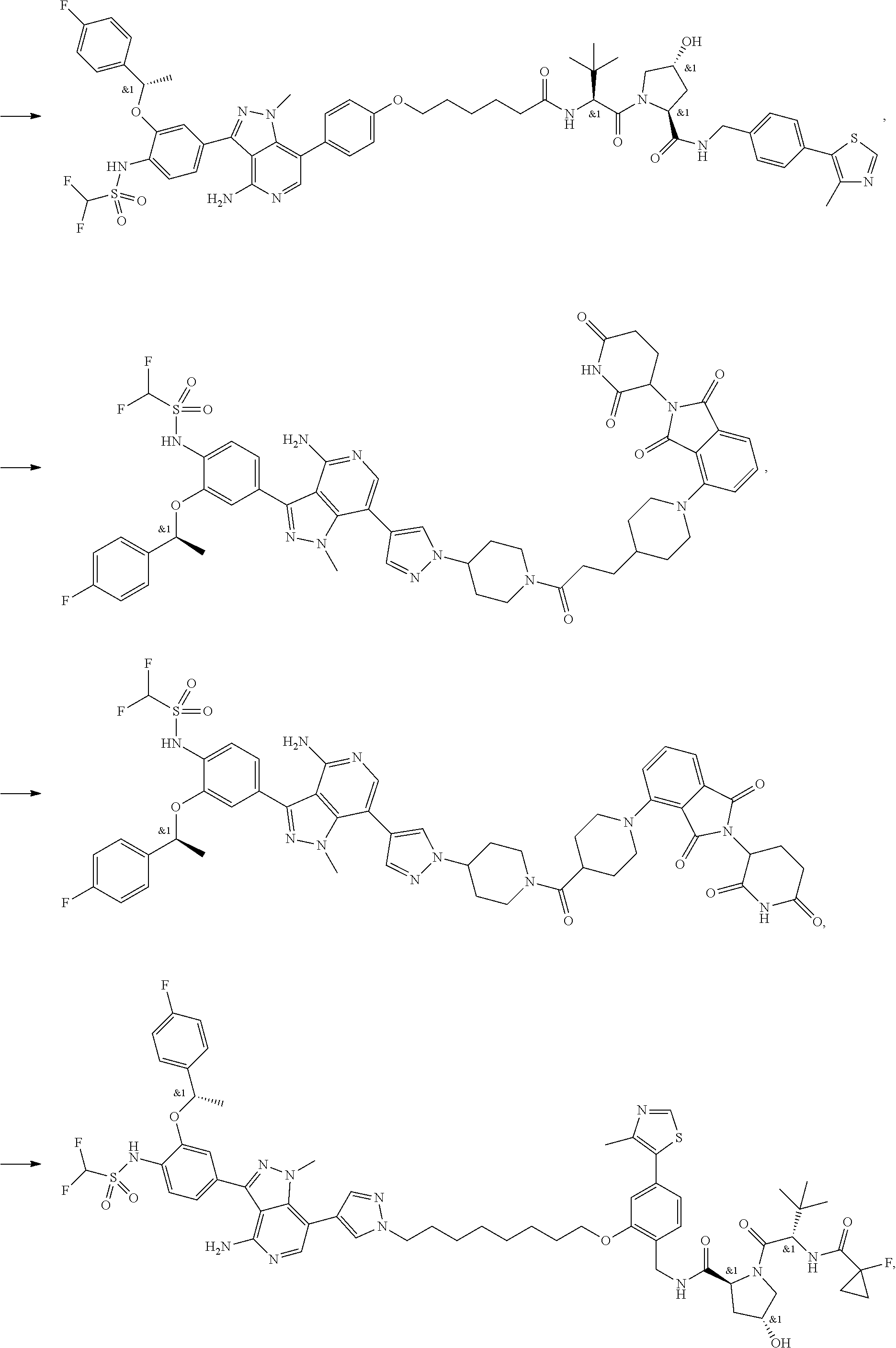 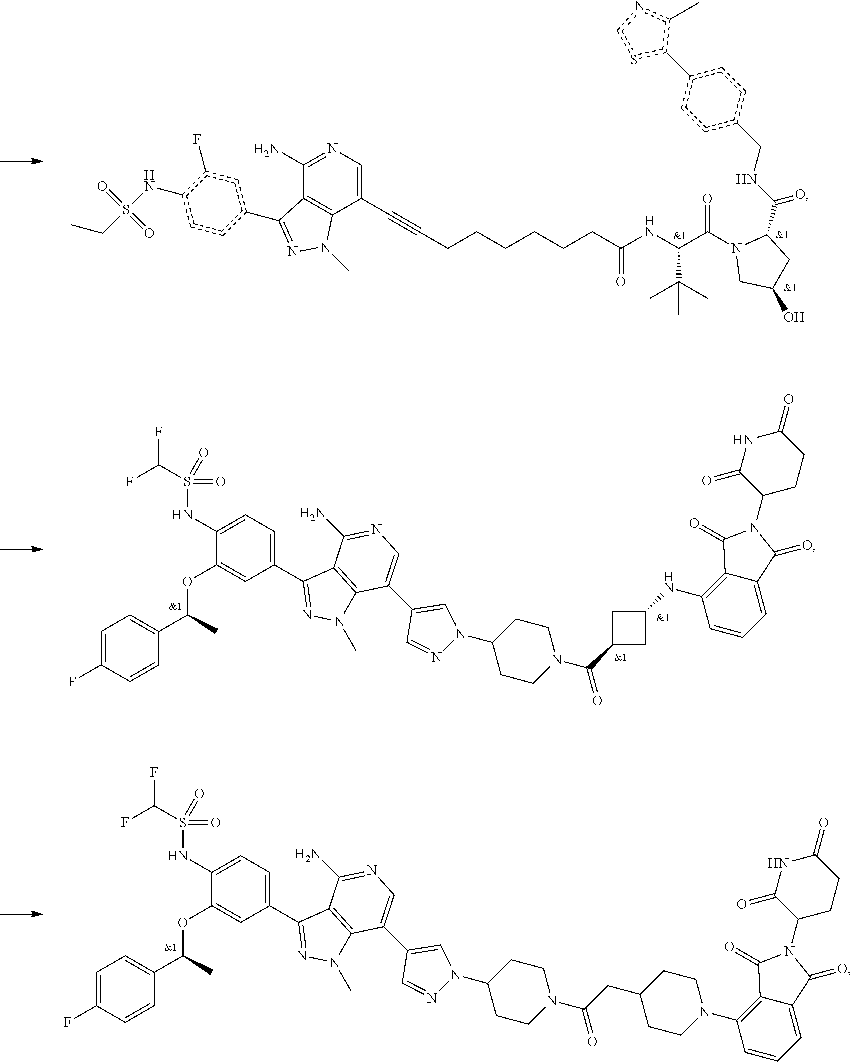 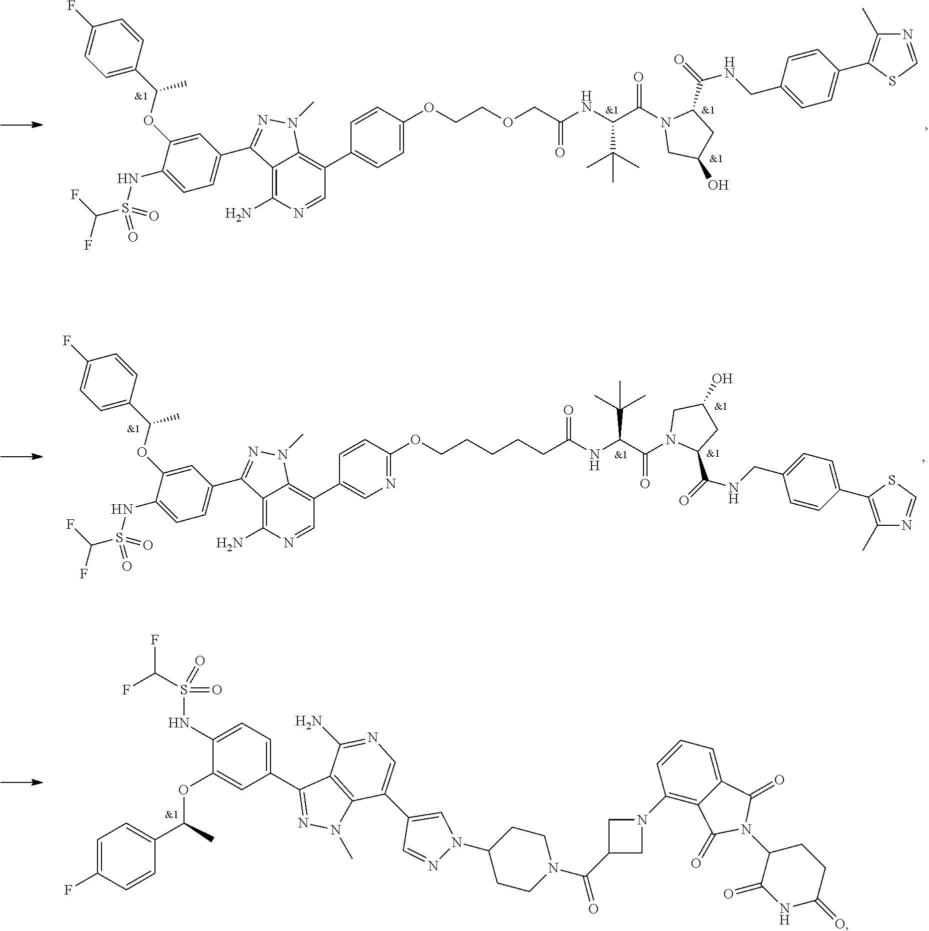 [SMILES: Cc1ncsc1-c1ccc(CNC(=O)[C@@H]2C[C@@H](O)CN2C(=O)[C@@H](NC(=O)COCCOc2ccc(-c3cnc(N)c4c(-c5ccc(NS(=O)(=O)C(F)F)c(O[C@@H](C)c6ccc(F)cc6)c5)nn(C)c34)cc2)C(C)(C)C)cc1 ; Cc1ncsc1-c1ccc(CNC(=O)[C@@H]2C[C@@H](O)CN2C(=O)[C@@H](NC(=O)CCCCCOc2ccc(-c3cnc(N)c4c(-c5ccc(NS(=O)(=O)C(F)F)c(O[C@@H](C)c6ccc(F)cc6)c5)nn(C)c34)cn2)C(C)(C)C)cc1 ; C[C@H](Oc1cc(-c2nn(C)c3c(-c4cnn(C5CCN(C(=O)C6CN(c7cccc8c7C(=O)N(C7CCC(=O)NC7=O)C8=O)C6)CC5)c4)cnc(N)c23)ccc1NS(=O)(=O)C(F)F)c1ccc(F)cc1] 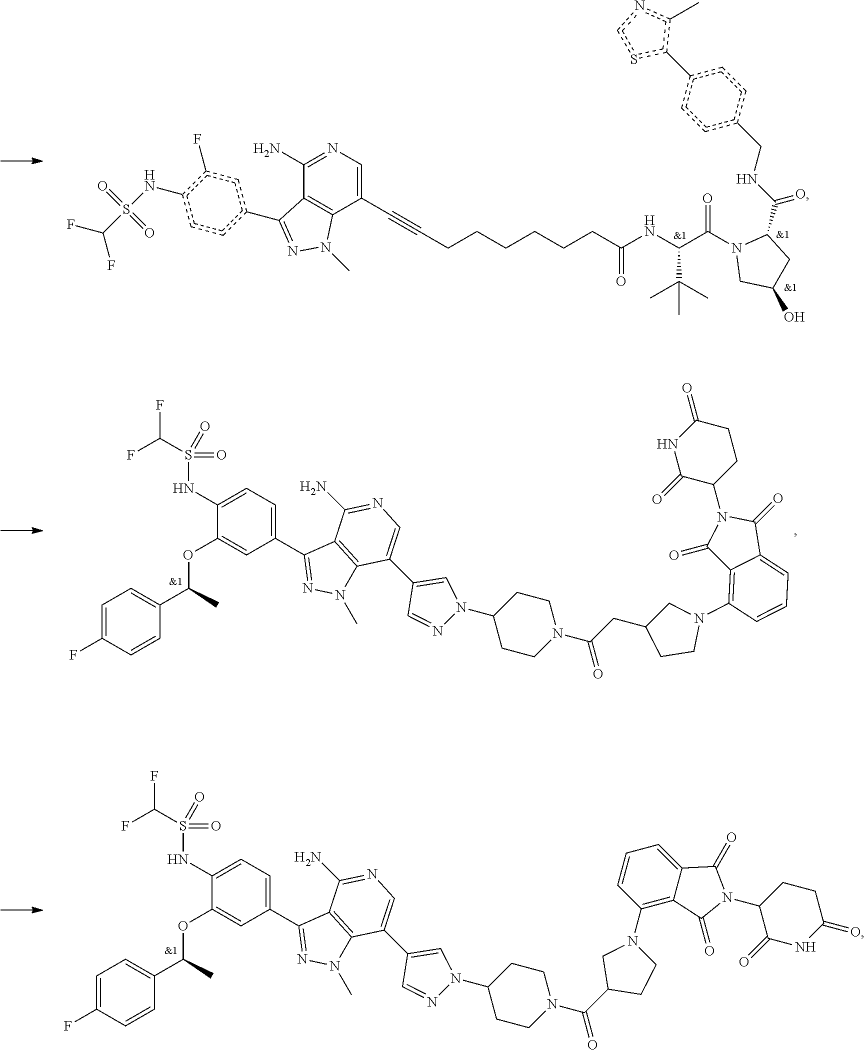 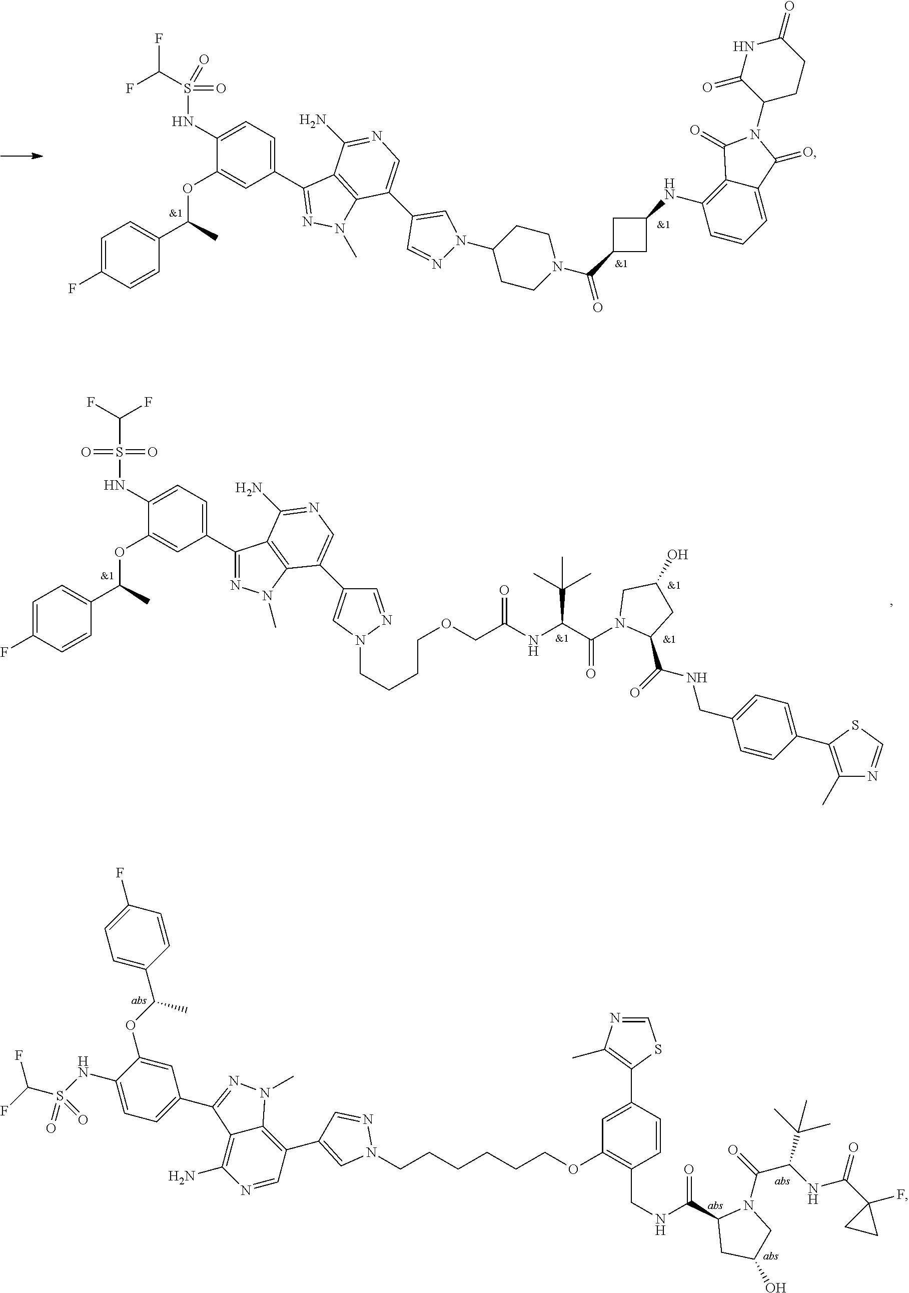 [SMILES: C[C@H](Oc1cc(-c2nn(C)c3c(-c4cnn(C5CCN(C(=O)[C@H]6C[C@@H](Nc7cccc8c7C(=O)N(C7CCC(=O)NC7=O)C8=O)C6)CC5)c4)cnc(N)c23)ccc1NS(=O)(=O)C(F)F)c1ccc(F)cc1 ; Cc1ncsc1-c1ccc(CNC(=O)[C@@H]2C[C@@H](O)CN2C(=O)[C@@H](NC(=O)COCCCCn2cc(-c3cnc(N)c4c(-c5ccc(NS(=O)(=O)C(F)F)c(O[C@@H](C)c6ccc(F)cc6)c5)nn(C)c34)cn2)C(C)(C)C)cc1 ; Cc1ncsc1-c1ccc(CNC(=O)[C@@H]2C[C@@H](O)CN2C(=O)[C@@H](NC(=O)C2(F)CC2)C(C)(C)C)c(OCCCCCCn2cc(-c3cnc(N)c4c(-c5ccc(NS(=O)(=O)C(F)F)c(O[C@@H](C)c6ccc(F)cc6)c5)nn(C)c34)cn2)c1] 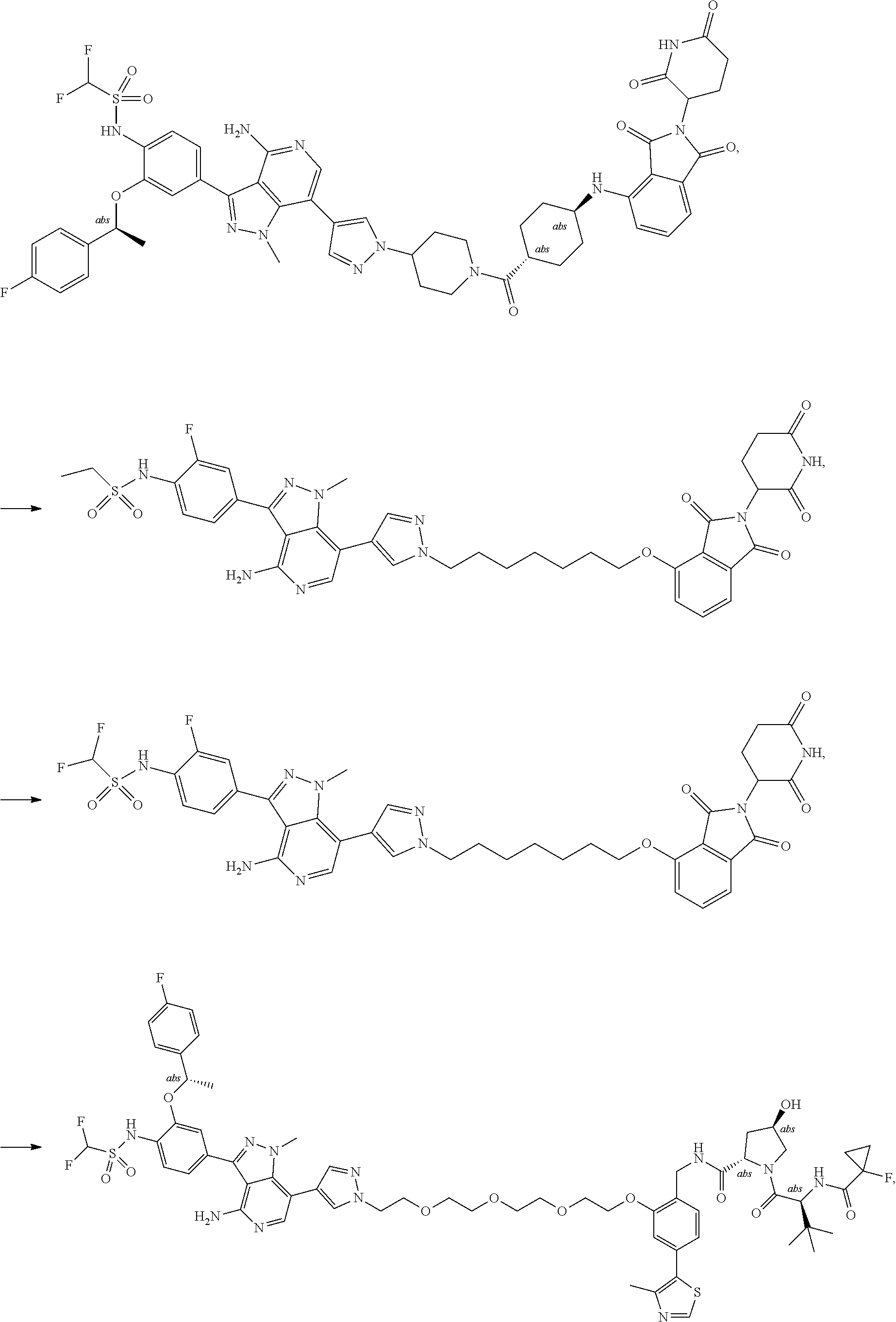 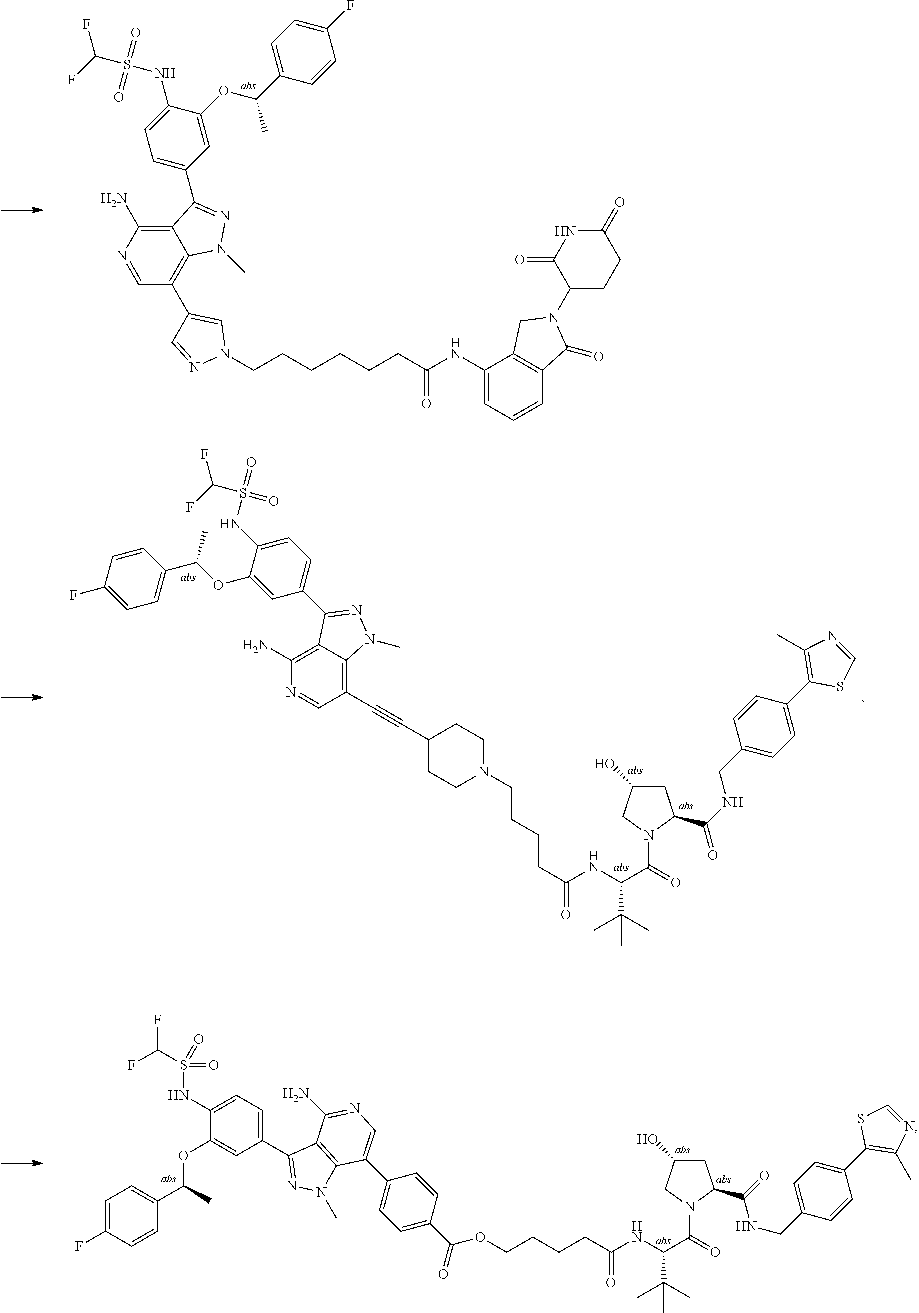 [SMILES: C[C@H](Oc1cc(-c2nn(C)c3c(-c4cnn(CCCCCCC(=O)Nc5cccc6c5CN(C5CCC(=O)NC5=O)C6=O)c4)cnc(N)c23)ccc1NS(=O)(=O)C(F)F)c1ccc(F)cc1 ; Cc1ncsc1-c1ccc(CNC(=O)[C@@H]2C[C@@H](O)CN2C(=O)[C@@H](NC(=O)CCCCN2CCC(C#Cc3cnc(N)c4c(-c5ccc(NS(=O)(=O)C(F)F)c(O[C@@H](C)c6ccc(F)cc6)c5)nn(C)c34)CC2)C(C)(C)C)cc1 ; Cc1ncsc1-c1ccc(CNC(=O)[C@@H]2C[C@@H](O)CN2C(=O)[C@@H](NC(=O)CCCCOC(=O)c2ccc(-c3cnc(N)c4c(-c5ccc(NS(=O)(=O)C(F)F)c(O[C@@H](C)c6ccc(F)cc6)c5)nn(C)c34)cc2)C(C)(C)C)cc1] 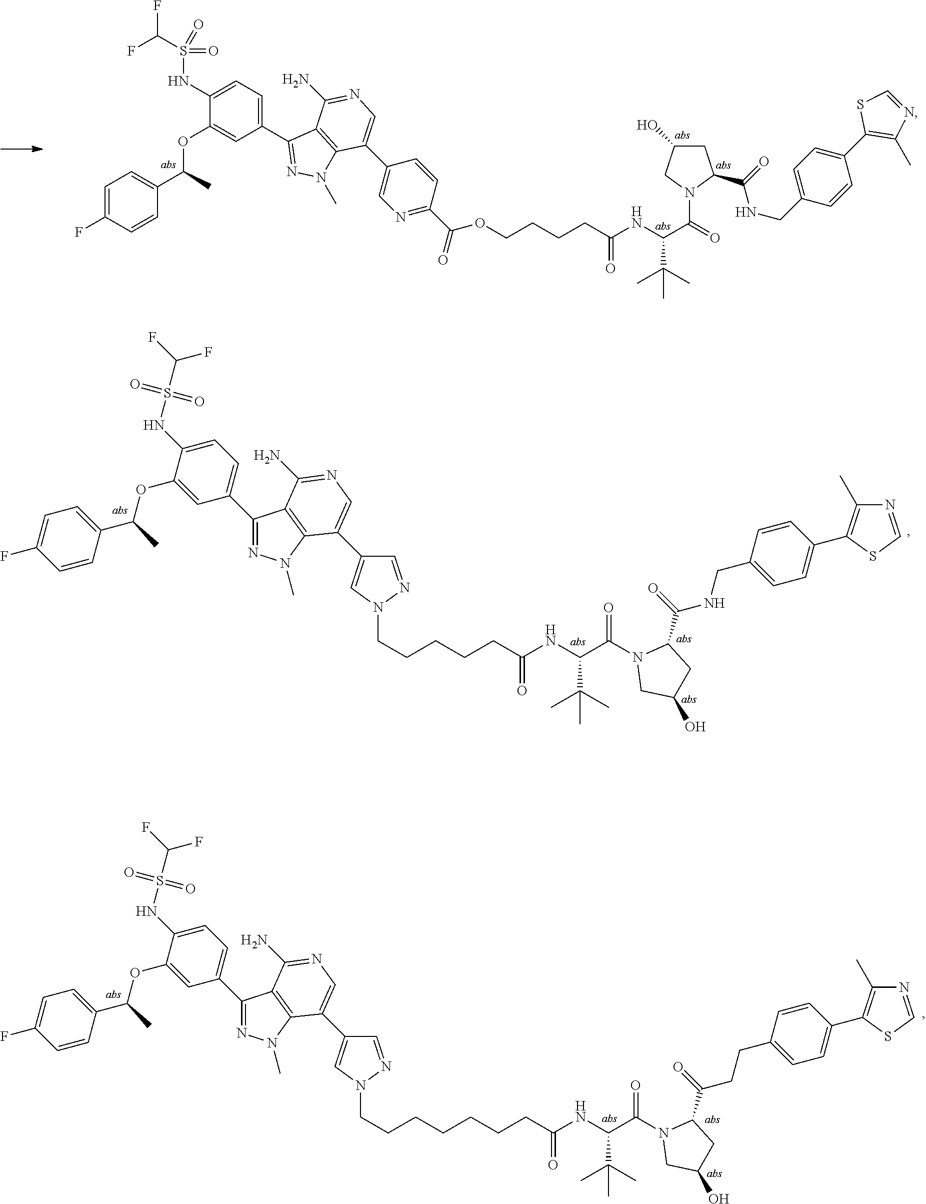 [SMILES: Cc1ncsc1-c1ccc(CNC(=O)[C@@H]2C[C@@H](O)CN2C(=O)[C@@H](NC(=O)CCCCOC(=O)c2ccc(-c3cnc(N)c4c(-c5ccc(NS(=O)(=O)C(F)F)c(O[C@@H](C)c6ccc(F)cc6)c5)nn(C)c34)cn2)C(C)(C)C)cc1 ; Cc1ncsc1-c1ccc(CNC(=O)[C@@H]2C[C@@H](O)CN2C(=O)[C@@H](NC(=O)CCCCCn2cc(-c3cnc(N)c4c(-c5ccc(NS(=O)(=O)C(F)F)c(O[C@@H](C)c6ccc(F)cc6)c5)nn(C)c34)cn2)C(C)(C)C)cc1 ; Cc1ncsc1-c1ccc(CCC(=O)[C@@H]2C[C@@H](O)CN2C(=O)[C@@H](NC(=O)CCCCCCCn2cc(-c3cnc(N)c4c(-c5ccc(NS(=O)(=O)C(F)F)c(O[C@@H](C)c6ccc(F)cc6)c5)nn(C)c34)cn2)C(C)(C)C)cc1] 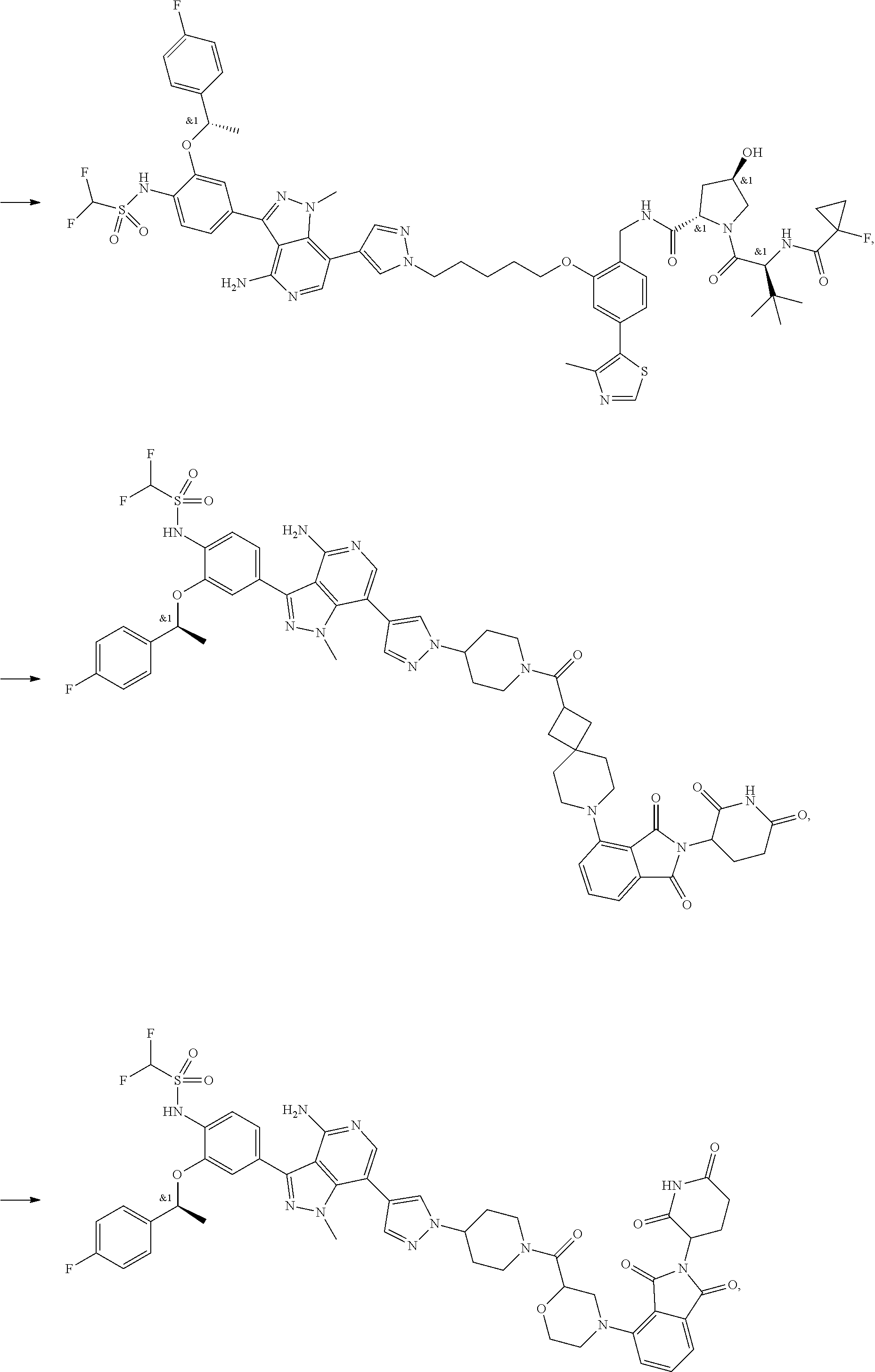 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCn2cc(-c3cnc(N)c4c(-c5ccc(NS(=O)(=O)C(F)F)c(O[C@@H](C)c6ccc(F)cc6)c5)nn(C)c34)cn2)c1 ; C[C@H](Oc1cc(-c2nn(C)c3c(-c4cnn(C5CCN(C(=O)C6CC7(CCN(c8cccc9c8C(=O)N(C8CCC(=O)NC8=O)C9=O)CC7)C6)CC5)c4)cnc(N)c23)ccc1NS(=O)(=O)C(F)F)c1ccc(F)cc1 ; C[C@H](Oc1cc(-c2nn(C)c3c(-c4cnn(C5CCN(C(=O)C6CN(c7cccc8c7C(=O)N(C7CCC(=O)NC7=O)C8=O)CCO6)CC5)c4)cnc(N)c23)ccc1NS(=O)(=O)C(F)F)c1ccc(F)cc1] 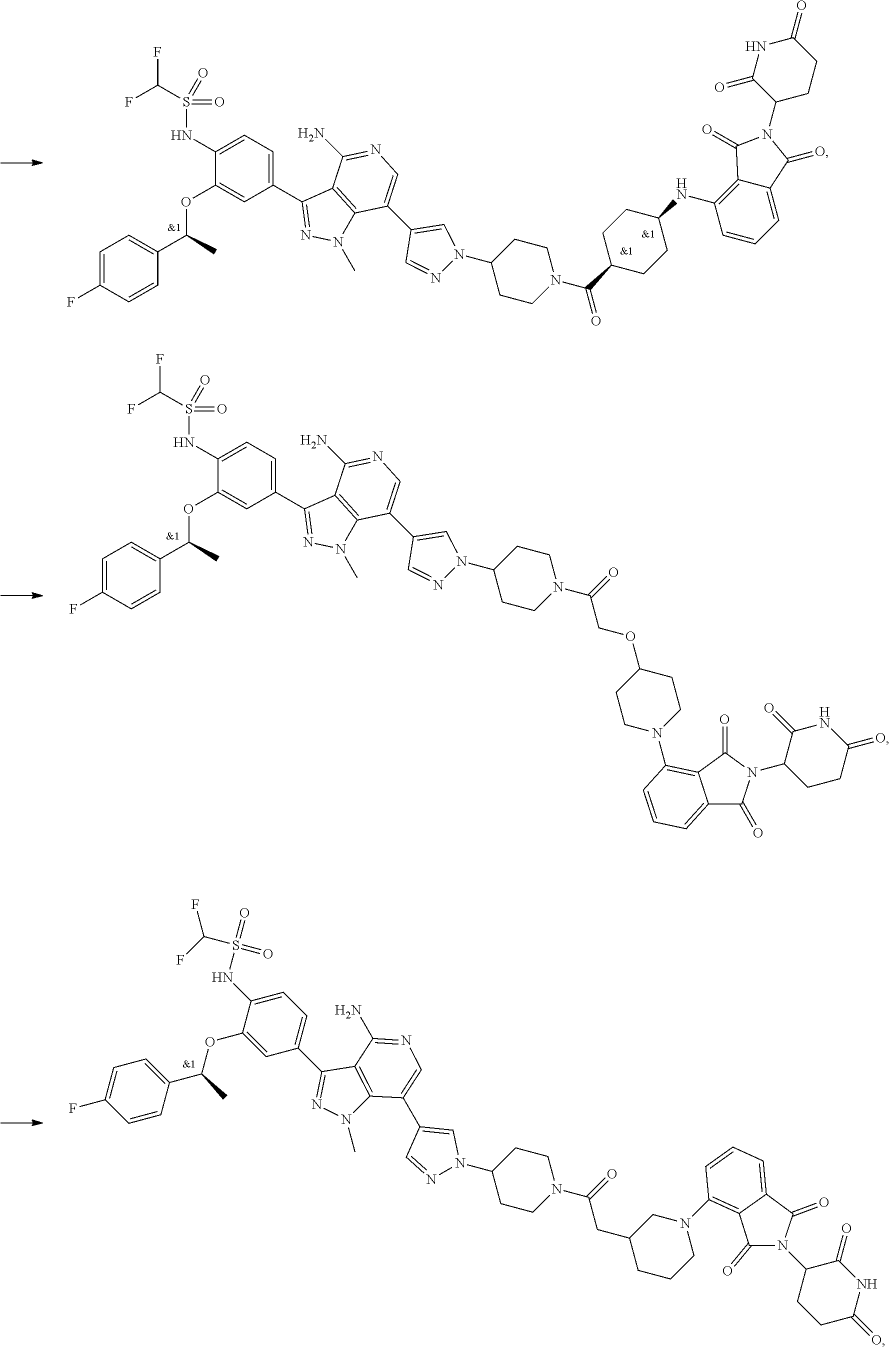 [SMILES: C[C@H](Oc1cc(-c2nn(C)c3c(-c4cnn(C5CCN(C(=O)[C@H]6CC[C@@H](Nc7cccc8c7C(=O)N(C7CCC(=O)NC7=O)C8=O)CC6)CC5)c4)cnc(N)c23)ccc1NS(=O)(=O)C(F)F)c1ccc(F)cc1 ; C[C@H](Oc1cc(-c2nn(C)c3c(-c4cnn(C5CCN(C(=O)COC6CCN(c7cccc8c7C(=O)N(C7CCC(=O)NC7=O)C8=O)CC6)CC5)c4)cnc(N)c23)ccc1NS(=O)(=O)C(F)F)c1ccc(F)cc1 ; C[C@H](Oc1cc(-c2nn(C)c3c(-c4cnn(C5CCN(C(=O)CC6CCCN(c7cccc8c7C(=O)N(C7CCC(=O)NC7=O)C8=O)C6)CC5)c4)cnc(N)c23)ccc1NS(=O)(=O)C(F)F)c1ccc(F)cc1] 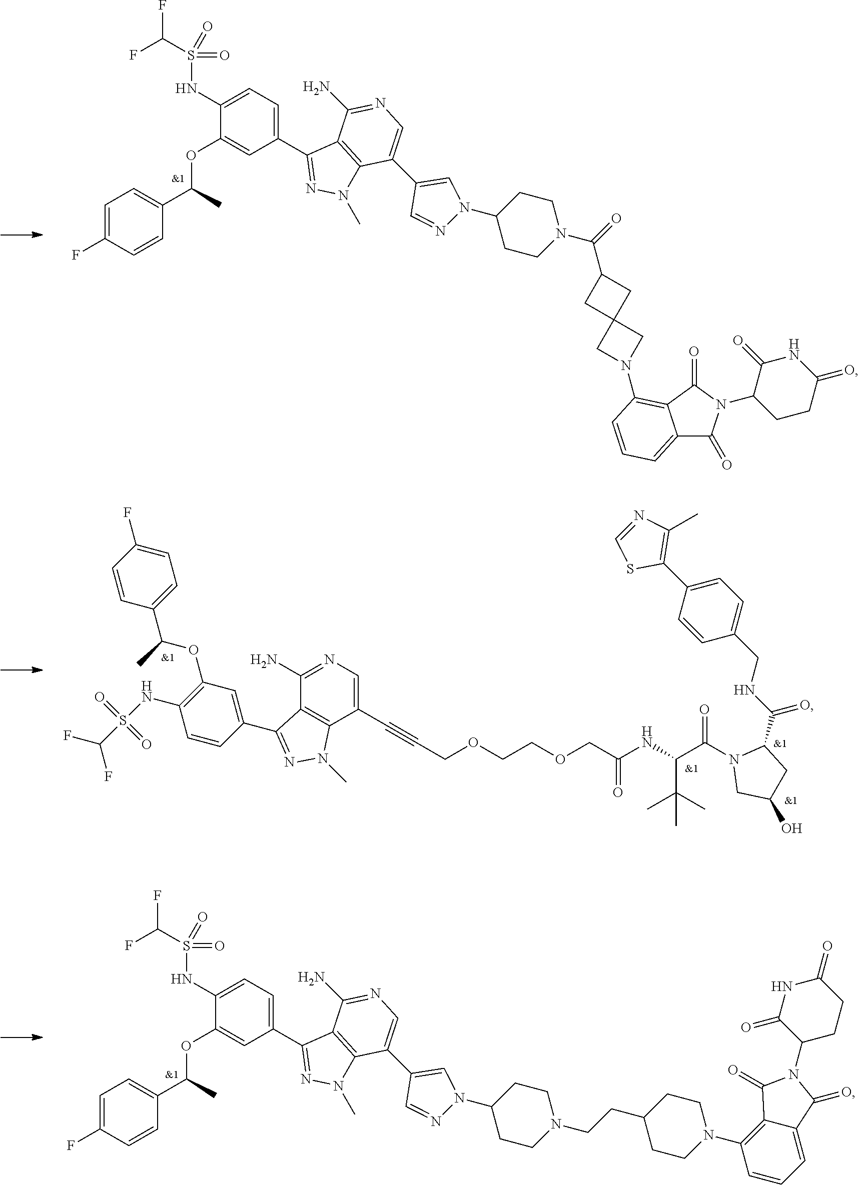 [SMILES: C[C@H](Oc1cc(-c2nn(C)c3c(-c4cnn(C5CCN(C(=O)C6CC7(C6)CN(c6cccc8c6C(=O)N(C6CCC(=O)NC6=O)C8=O)C7)CC5)c4)cnc(N)c23)ccc1NS(=O)(=O)C(F)F)c1ccc(F)cc1 ; Cc1ncsc1-c1ccc(CNC(=O)[C@@H]2C[C@@H](O)CN2C(=O)[C@@H](NC(=O)COCCOCC#Cc2cnc(N)c3c(-c4ccc(NS(=O)(=O)C(F)F)c(O[C@@H](C)c5ccc(F)cc5)c4)nn(C)c23)C(C)(C)C)cc1 ; C[C@H](Oc1cc(-c2nn(C)c3c(-c4cnn(C5CCN(CCC6CCN(c7cccc8c7C(=O)N(C7CCC(=O)NC7=O)C8=O)CC6)CC5)c4)cnc(N)c23)ccc1NS(=O)(=O)C(F)F)c1ccc(F)cc1] 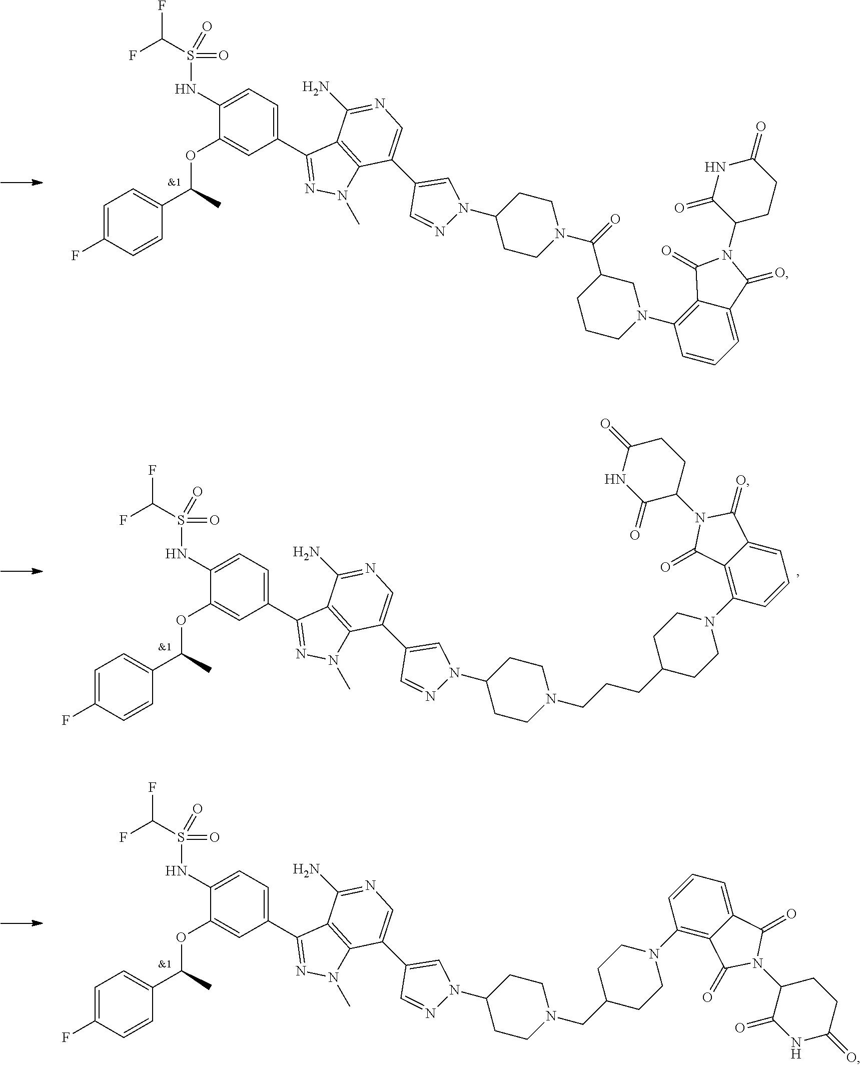 [SMILES: C[C@H](Oc1cc(-c2nn(C)c3c(-c4cnn(C5CCN(C(=O)C6CCCN(c7cccc8c7C(=O)N(C7CCC(=O)NC7=O)C8=O)C6)CC5)c4)cnc(N)c23)ccc1NS(=O)(=O)C(F)F)c1ccc(F)cc1 ; C[C@H](Oc1cc(-c2nn(C)c3c(-c4cnn(C5CCN(CCCC6CCN(c7cccc8c7C(=O)N(C7CCC(=O)NC7=O)C8=O)CC6)CC5)c4)cnc(N)c23)ccc1NS(=O)(=O)C(F)F)c1ccc(F)cc1 ; C[C@H](Oc1cc(-c2nn(C)c3c(-c4cnn(C5CCN(CC6CCN(c7cccc8c7C(=O)N(C7CCC(=O)NC7=O)C8=O)CC6)CC5)c4)cnc(N)c23)ccc1NS(=O)(=O)C(F)F)c1ccc(F)cc1] 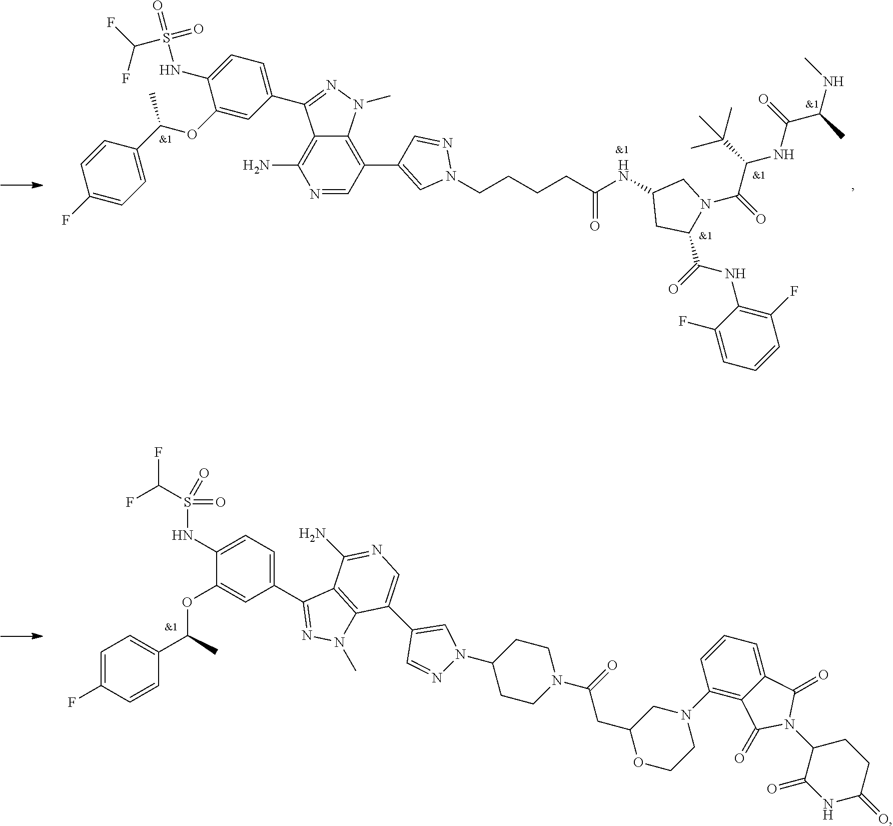 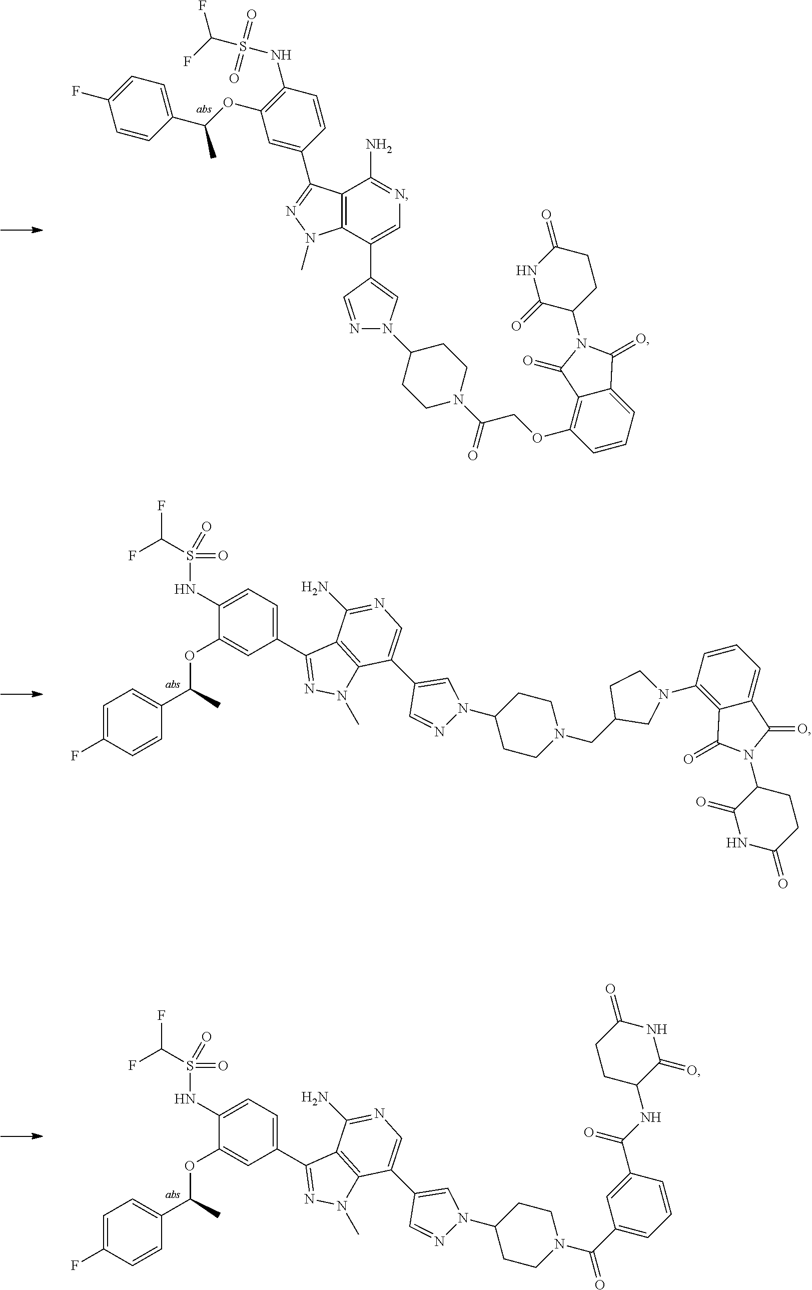 [SMILES: C[C@H](Oc1cc(-c2nn(C)c3c(-c4cnn(C5CCN(C(=O)COc6cccc7c6C(=O)N(C6CCC(=O)NC6=O)C7=O)CC5)c4)cnc(N)c23)ccc1NS(=O)(=O)C(F)F)c1ccc(F)cc1 ; C[C@H](Oc1cc(-c2nn(C)c3c(-c4cnn(C5CCN(CC6CCN(c7cccc8c7C(=O)N(C7CCC(=O)NC7=O)C8=O)C6)CC5)c4)cnc(N)c23)ccc1NS(=O)(=O)C(F)F)c1ccc(F)cc1 ; C[C@H](Oc1cc(-c2nn(C)c3c(-c4cnn(C5CCN(C(=O)c6cccc(C(=O)NC7CCC(=O)NC7=O)c6)CC5)c4)cnc(N)c23)ccc1NS(=O)(=O)C(F)F)c1ccc(F)cc1] 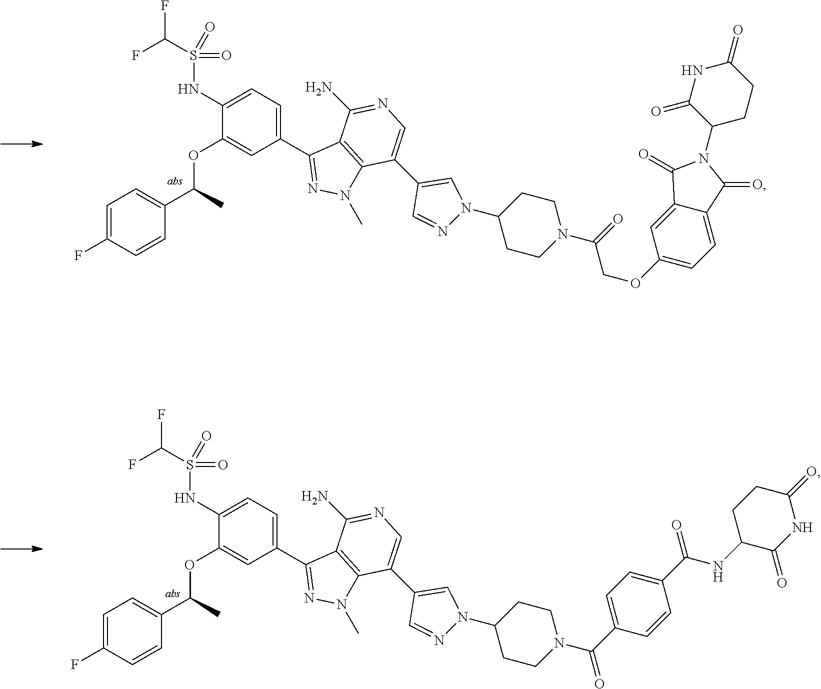 [SMILES: C[C@H](Oc1cc(-c2nn(C)c3c(-c4cnn(C5CCN(C(=O)COc6ccc7c(c6)C(=O)N(C6CCC(=O)NC6=O)C7=O)CC5)c4)cnc(N)c23)ccc1NS(=O)(=O)C(F)F)c1ccc(F)cc1 ; C[C@H](Oc1cc(-c2nn(C)c3c(-c4cnn(C5CCN(C(=O)c6ccc(C(=O)NC7CCC(=O)NC7=O)cc6)CC5)c4)cnc(N)c23)ccc1NS(=O)(=O)C(F)F)c1ccc(F)cc1] 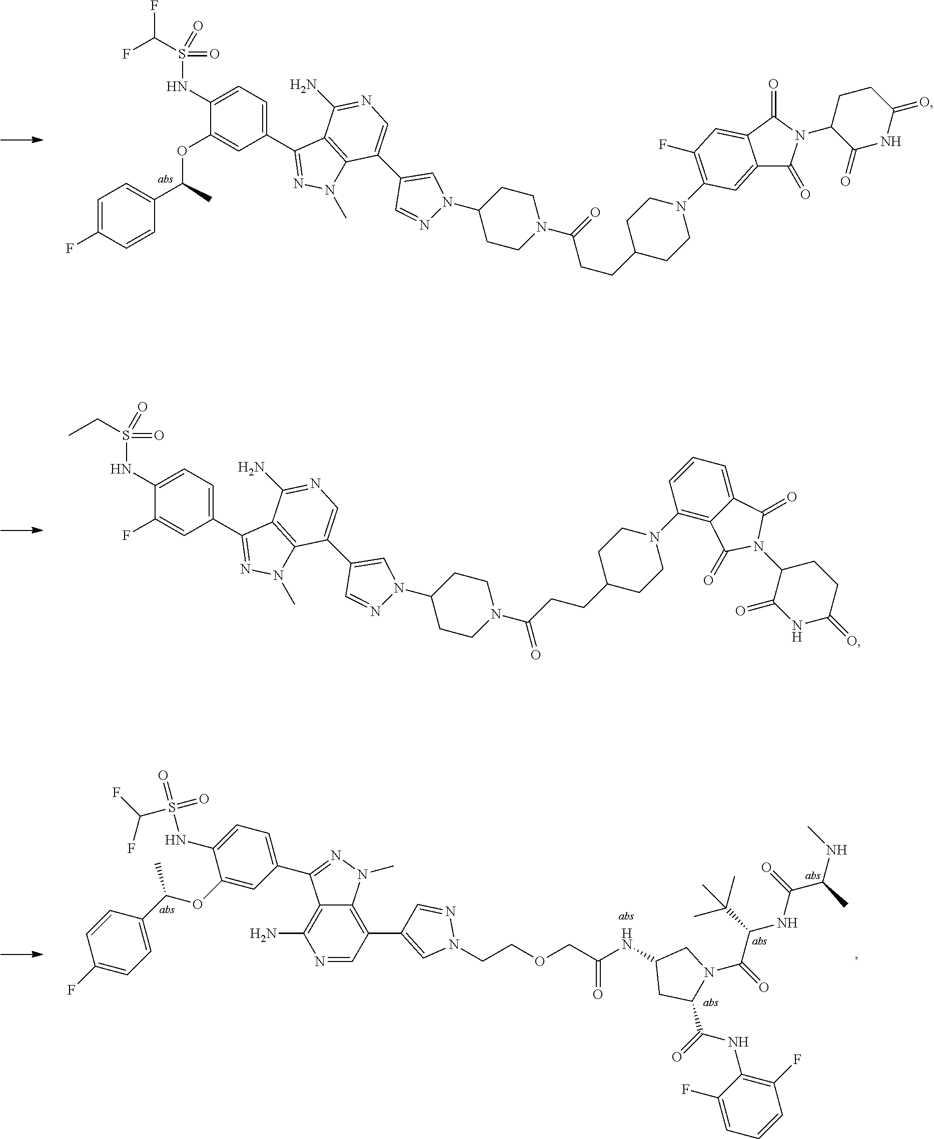 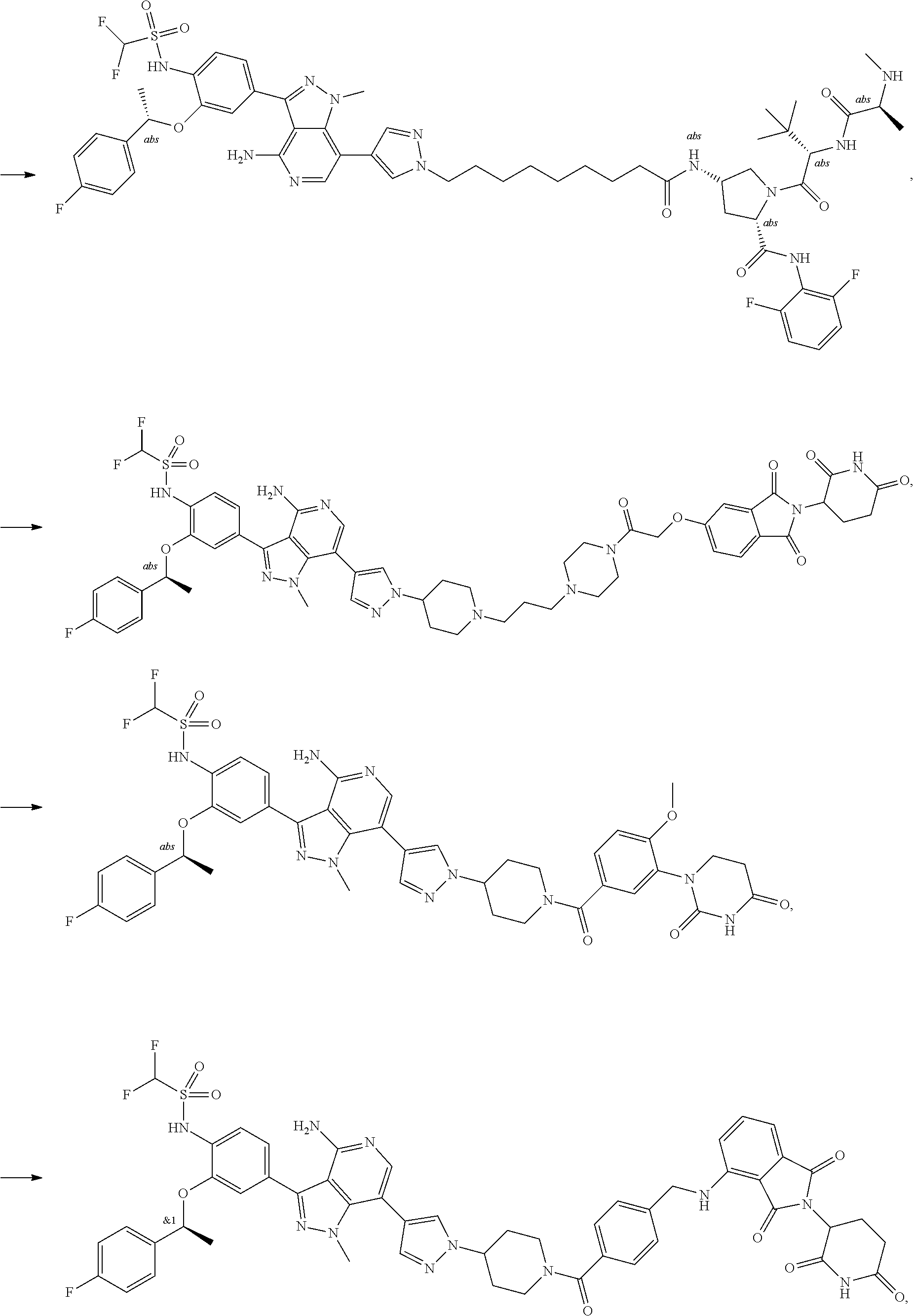 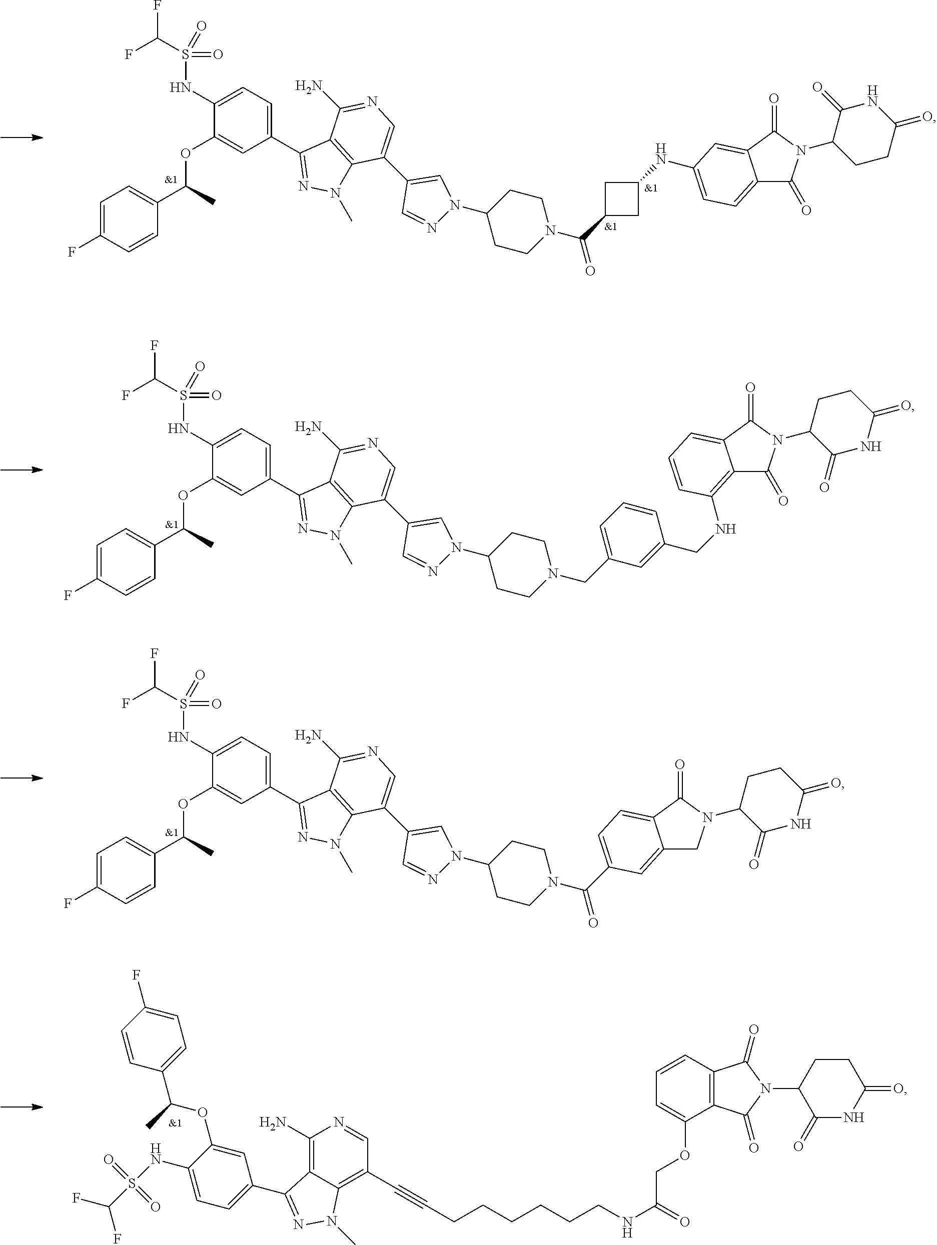 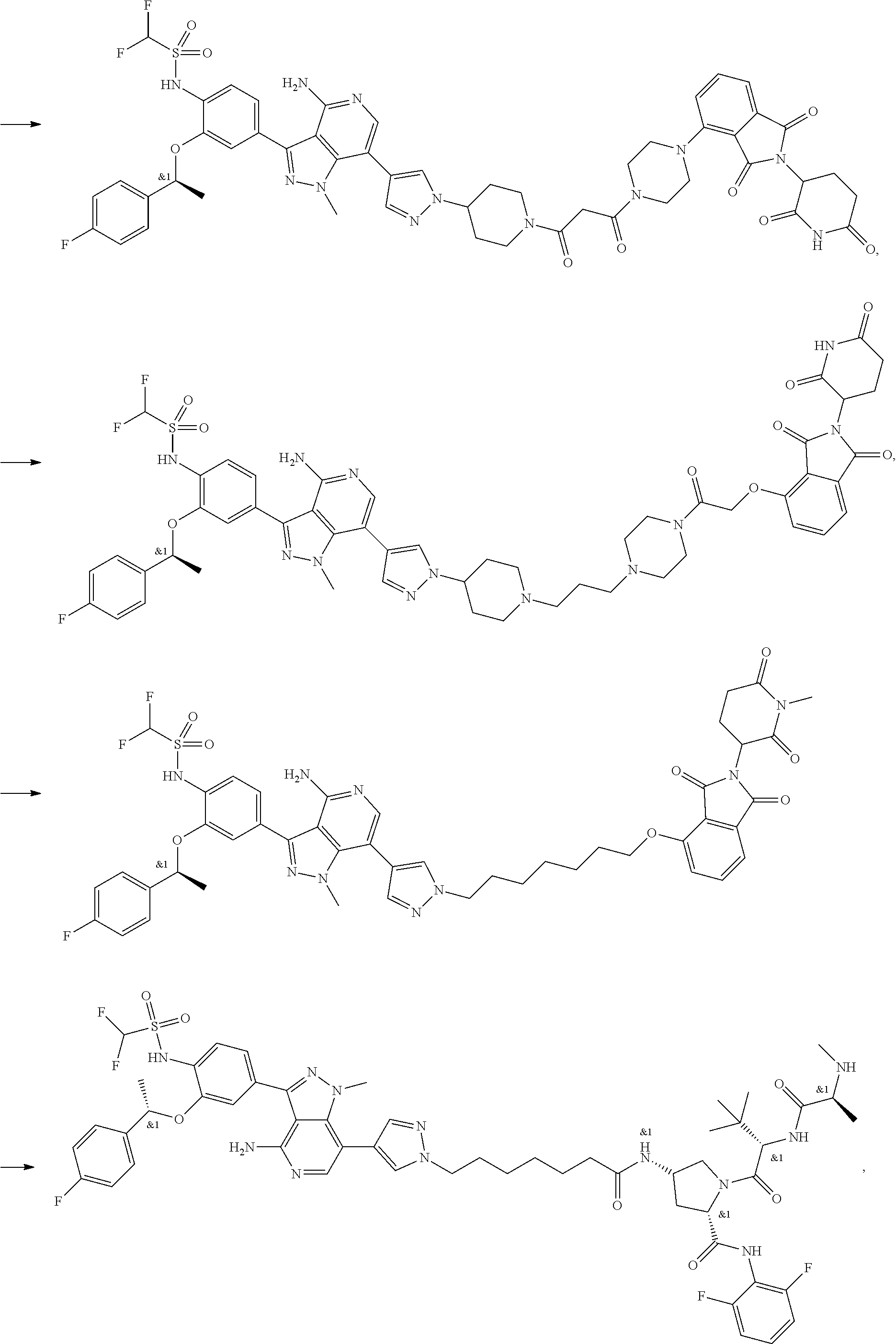 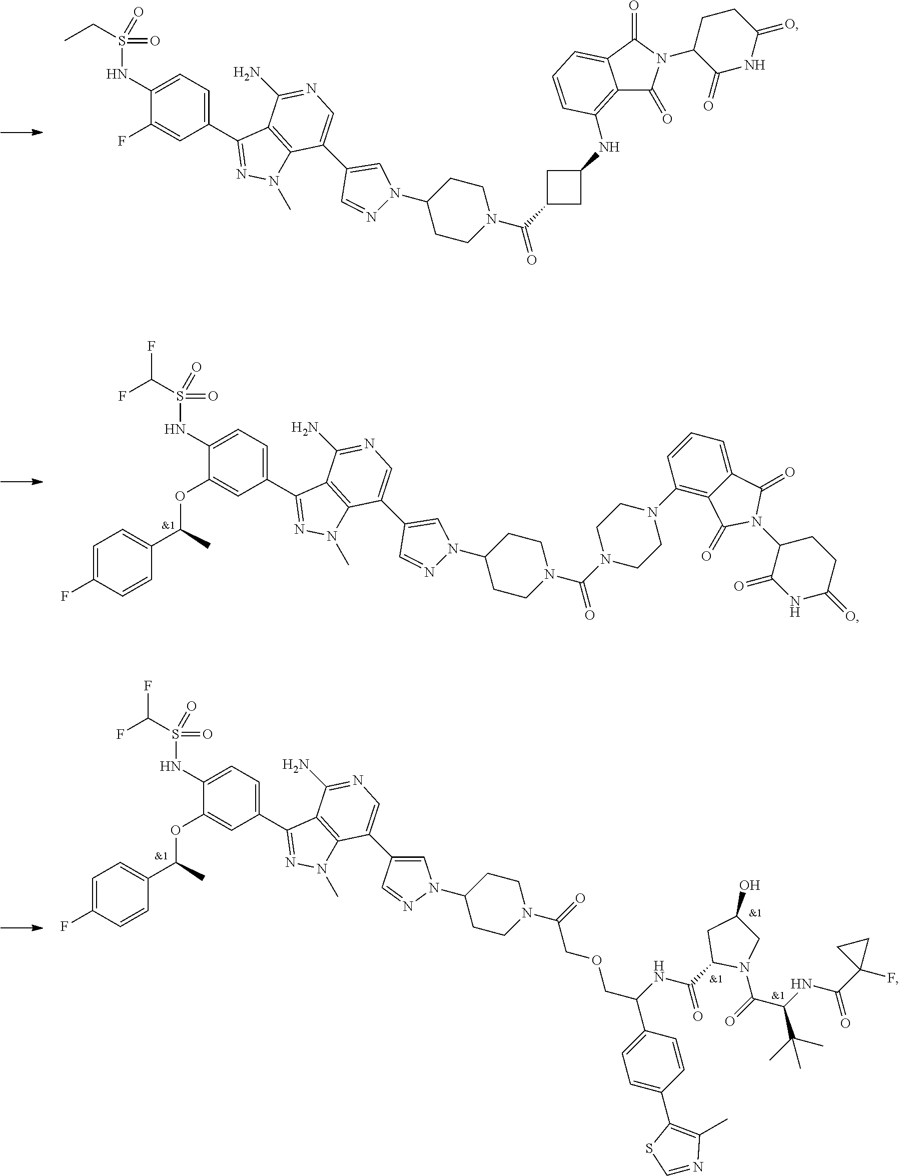 [SMILES: CCS(=O)(=O)Nc1ccc(-c2nn(C)c3c(-c4cnn(C5CCN(C(=O)[C@H]6C[C@H](Nc7cccc8c7C(=O)N(C7CCC(=O)NC7=O)C8=O)C6)CC5)c4)cnc(N)c23)cc1F ; C[C@H](Oc1cc(-c2nn(C)c3c(-c4cnn(C5CCN(C(=O)N6CCN(c7cccc8c7C(=O)N(C7CCC(=O)NC7=O)C8=O)CC6)CC5)c4)cnc(N)c23)ccc1NS(=O)(=O)C(F)F)c1ccc(F)cc1 ; Cc1ncsc1-c1ccc(C(COCC(=O)N2CCC(n3cc(-c4cnc(N)c5c(-c6ccc(NS(=O)(=O)C(F)F)c(O[C@@H](C)c7ccc(F)cc7)c6)nn(C)c45)cn3)CC2)NC(=O)[C@@H]2C[C@@H](O)CN2C(=O)[C@@H](NC(=O)C2(F)CC2)C(C)(C)C)cc1] 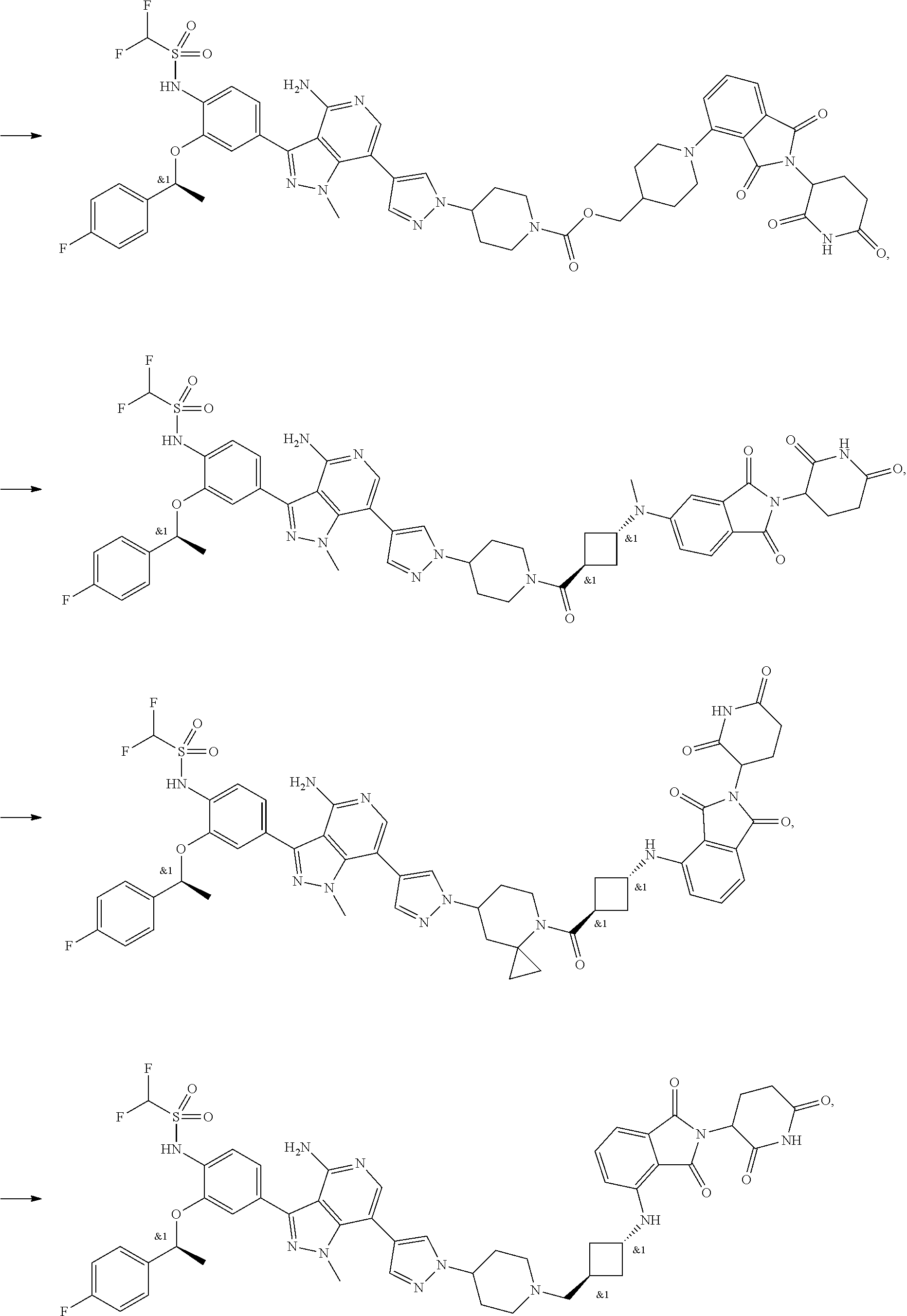 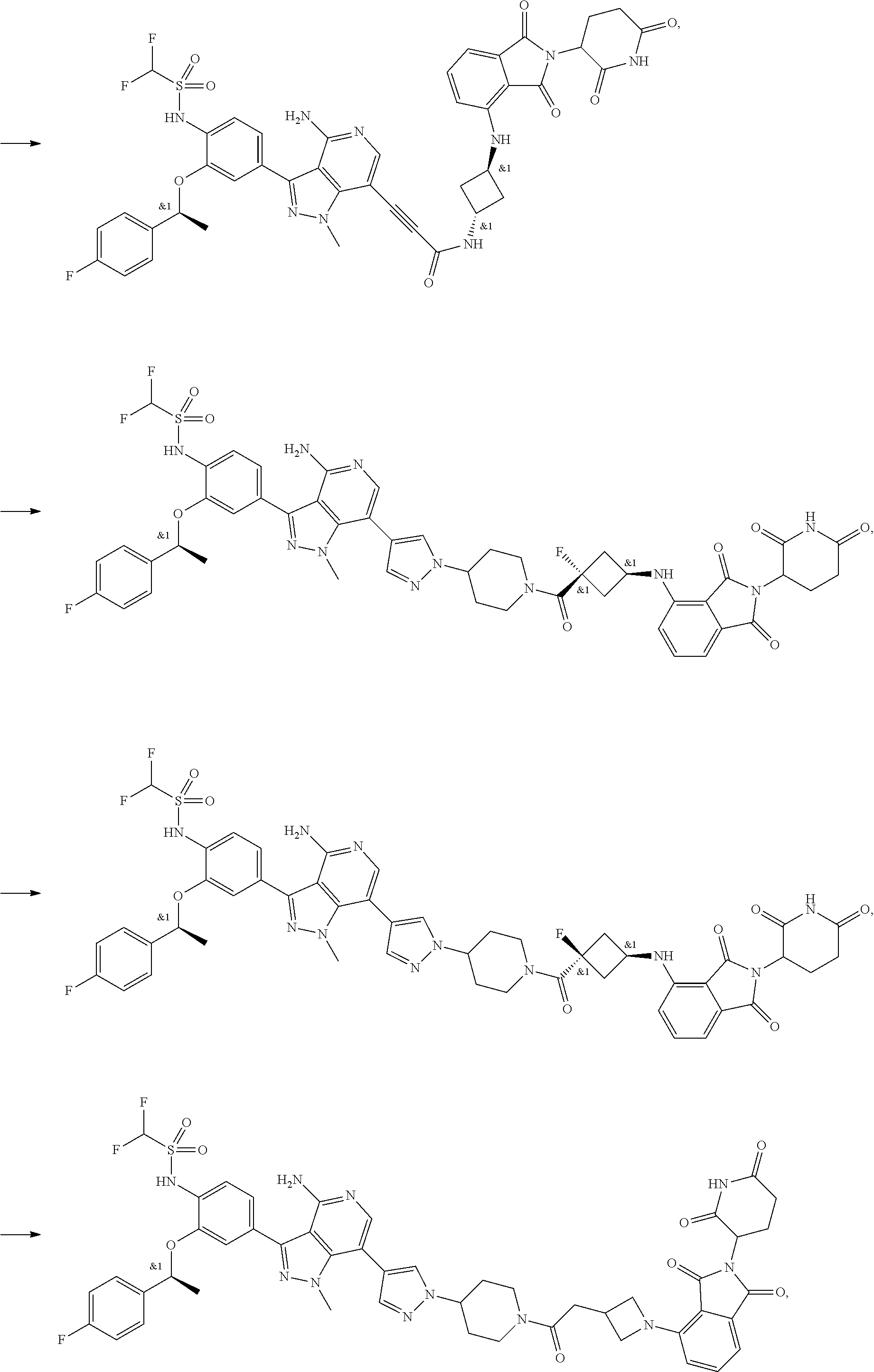 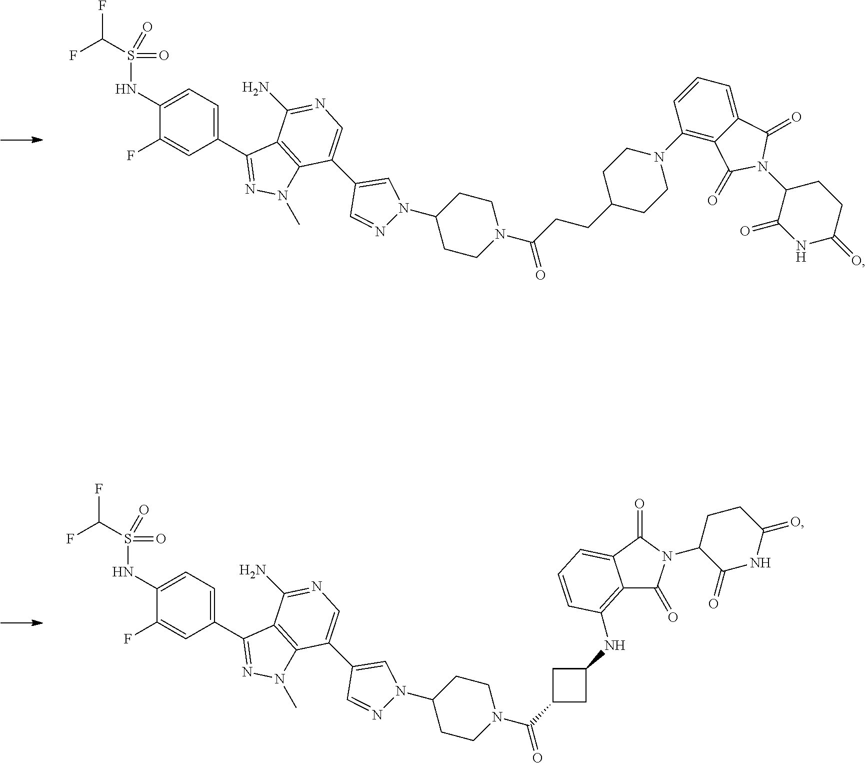 [SMILES: Cn1nc(-c2ccc(NS(=O)(=O)C(F)F)c(F)c2)c2c(N)ncc(-c3cnn(C4CCN(C(=O)CCC5CCN(c6cccc7c6C(=O)N(C6CCC(=O)NC6=O)C7=O)CC5)CC4)c3)c21 ; Cn1nc(-c2ccc(NS(=O)(=O)C(F)F)c(F)c2)c2c(N)ncc(-c3cnn(C4CCN(C(=O)[C@H]5C[C@H](Nc6cccc7c6C(=O)N(C6CCC(=O)NC6=O)C7=O)C5)CC4)c3)c21] 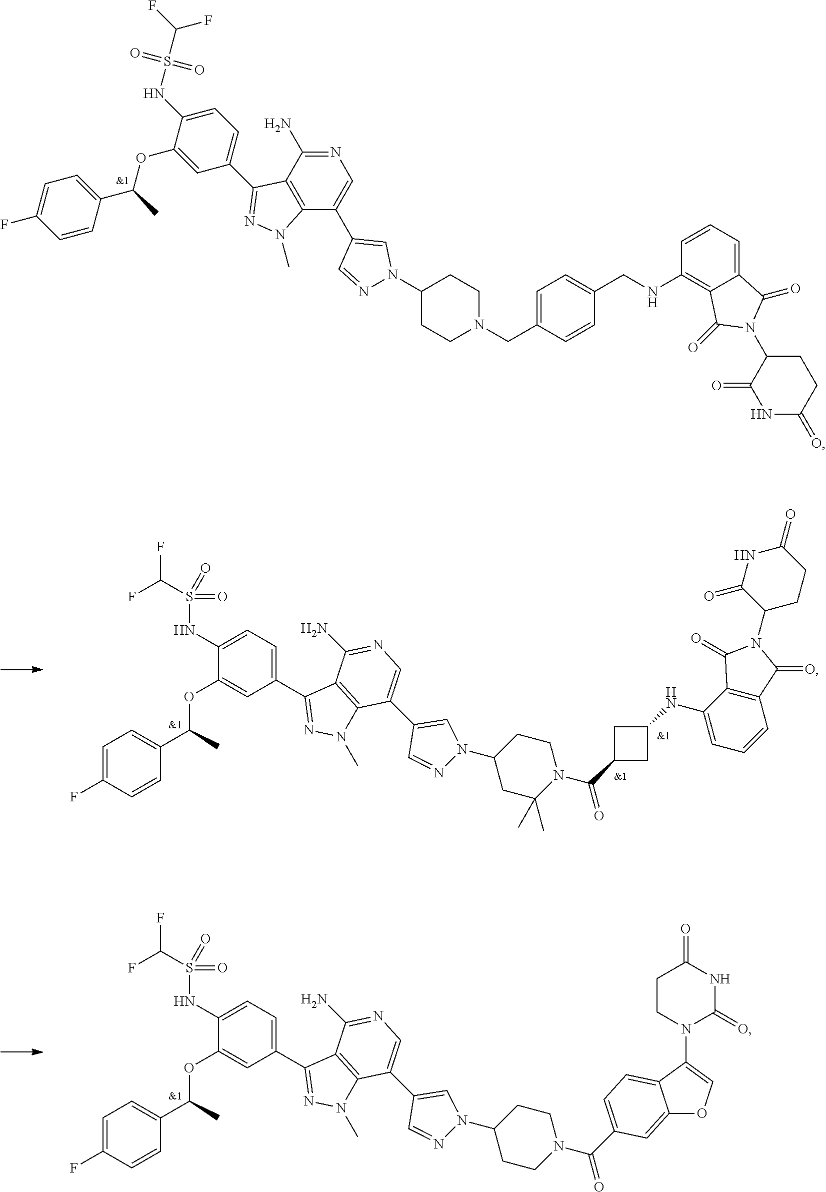 [SMILES: C[C@H](Oc1cc(-c2nn(C)c3c(-c4cnn(C5CCN(Cc6ccc(CNc7cccc8c7C(=O)N(C7CCC(=O)NC7=O)C8=O)cc6)CC5)c4)cnc(N)c23)ccc1NS(=O)(=O)C(F)F)c1ccc(F)cc1 ; C[C@H](Oc1cc(-c2nn(C)c3c(-c4cnn(C5CCN(C(=O)[C@H]6C[C@H](Nc7cccc8c7C(=O)N(C7CCC(=O)NC7=O)C8=O)C6)C(C)(C)C5)c4)cnc(N)c23)ccc1NS(=O)(=O)C(F)F)c1ccc(F)cc1 ; C[C@H](Oc1cc(-c2nn(C)c3c(-c4cnn(C5CCN(C(=O)c6ccc7c(N8CCC(=O)NC8=O)coc7c6)CC5)c4)cnc(N)c23)ccc1NS(=O)(=O)C(F)F)c1ccc(F)cc1] 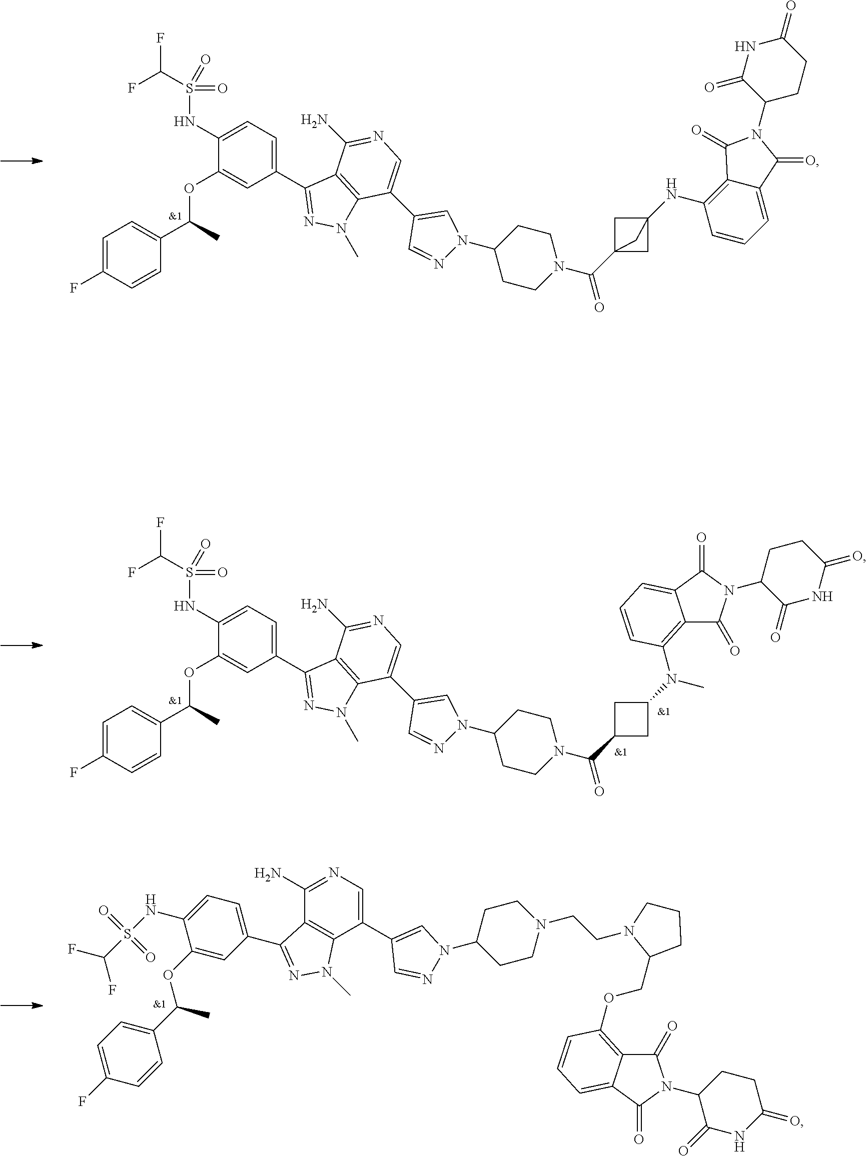 [SMILES: C[C@H](Oc1cc(-c2nn(C)c3c(-c4cnn(C5CCN(C(=O)C67CC(Nc8cccc9c8C(=O)N(C8CCC(=O)NC8=O)C9=O)(C6)C7)CC5)c4)cnc(N)c23)ccc1NS(=O)(=O)C(F)F)c1ccc(F)cc1 ; C[C@H](Oc1cc(-c2nn(C)c3c(-c4cnn(C5CCN(C(=O)[C@H]6C[C@H](N(C)c7cccc8c7C(=O)N(C7CCC(=O)NC7=O)C8=O)C6)CC5)c4)cnc(N)c23)ccc1NS(=O)(=O)C(F)F)c1ccc(F)cc1 ; C[C@H](Oc1cc(-c2nn(C)c3c(-c4cnn(C5CCN(CCN6CCCC6COc6cccc7c6C(=O)N(C6CCC(=O)NC6=O)C7=O)CC5)c4)cnc(N)c23)ccc1NS(=O)(=O)C(F)F)c1ccc(F)cc1] 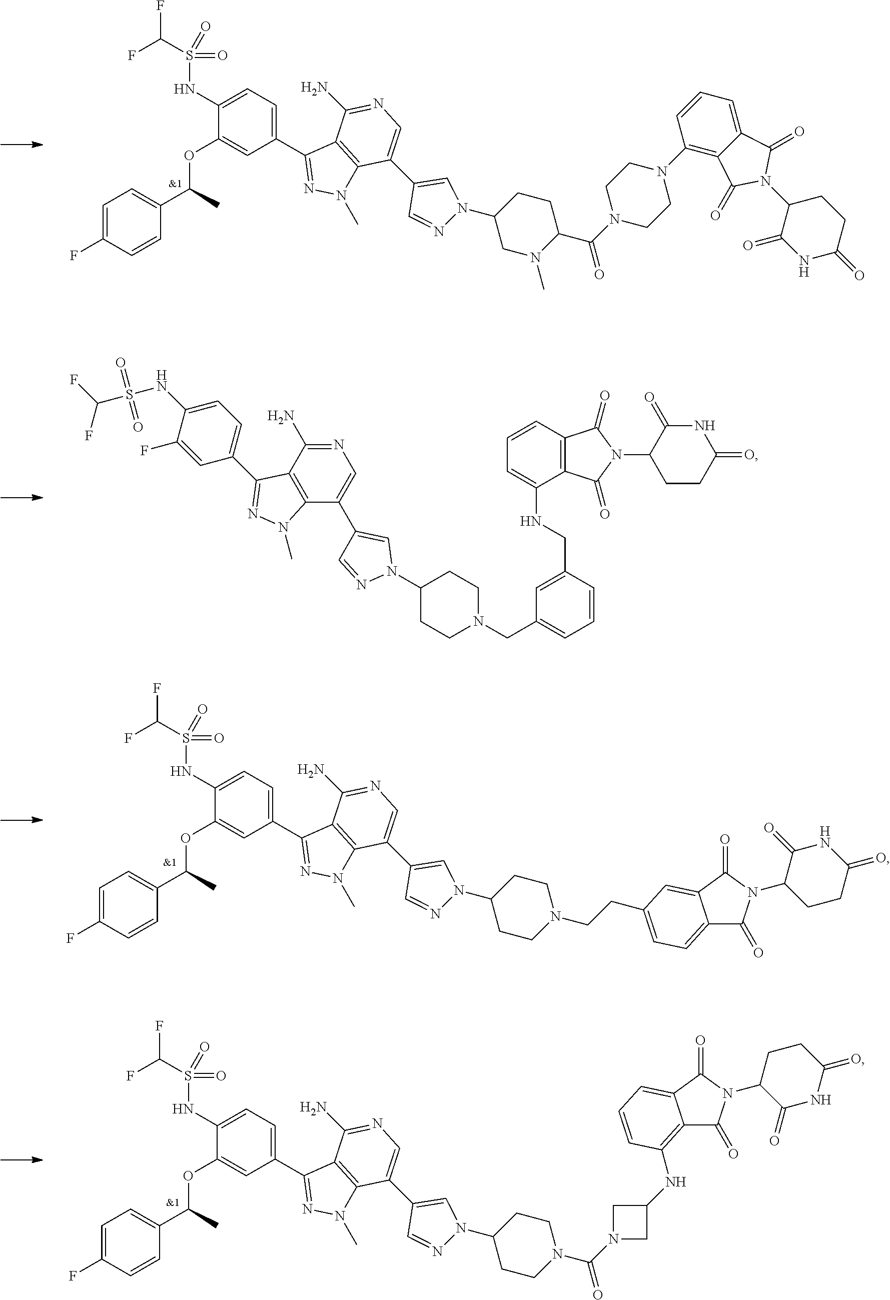 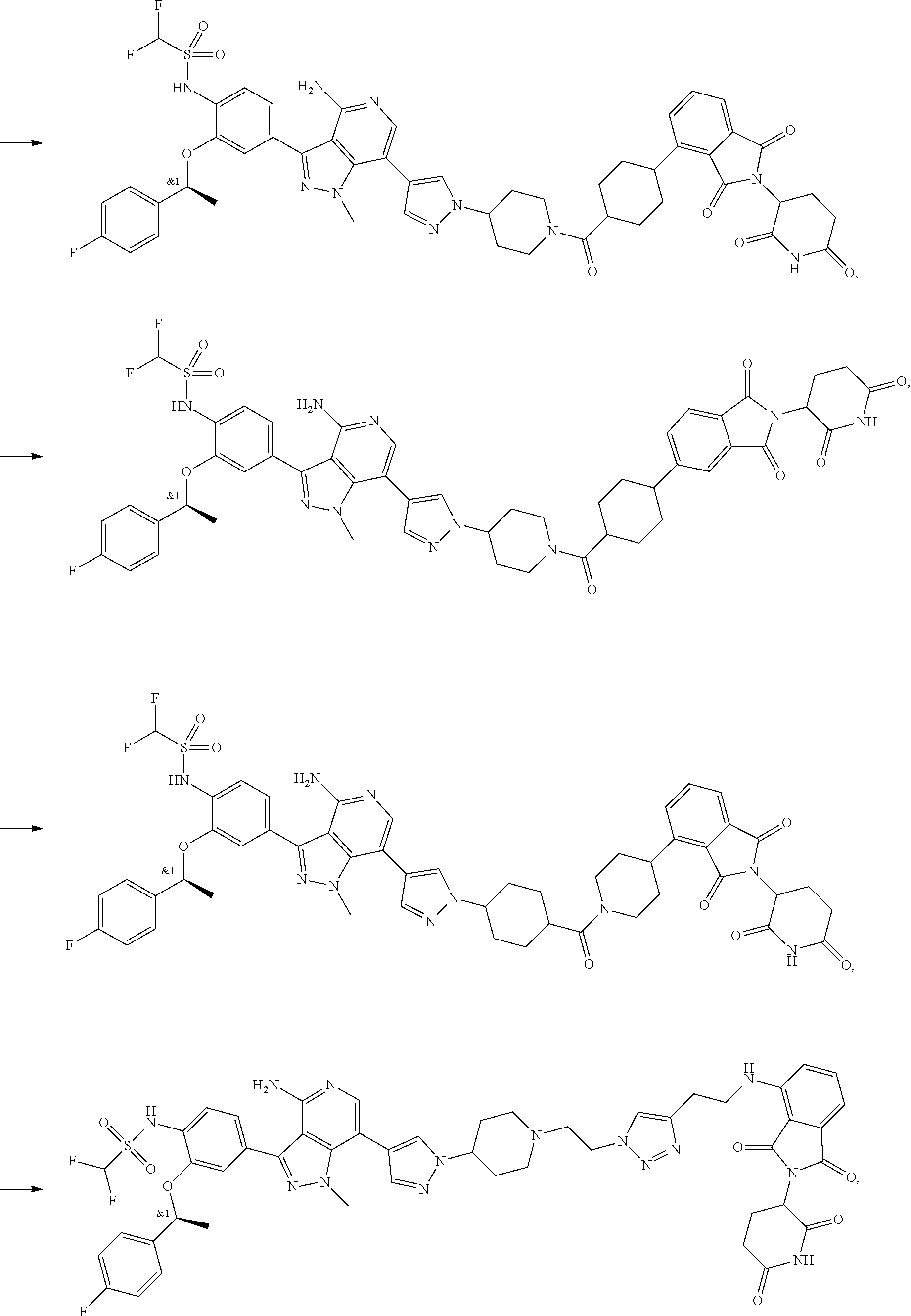 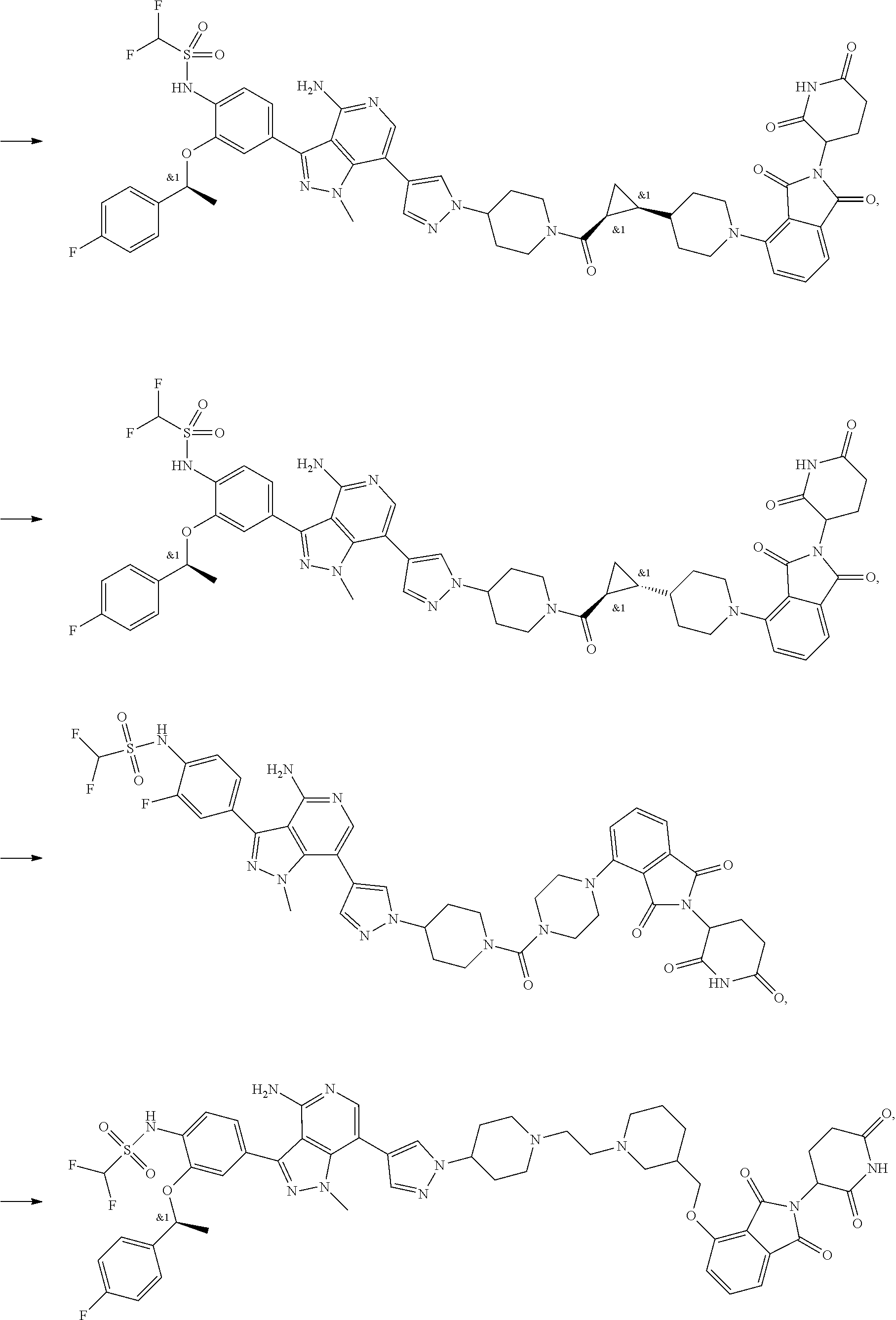 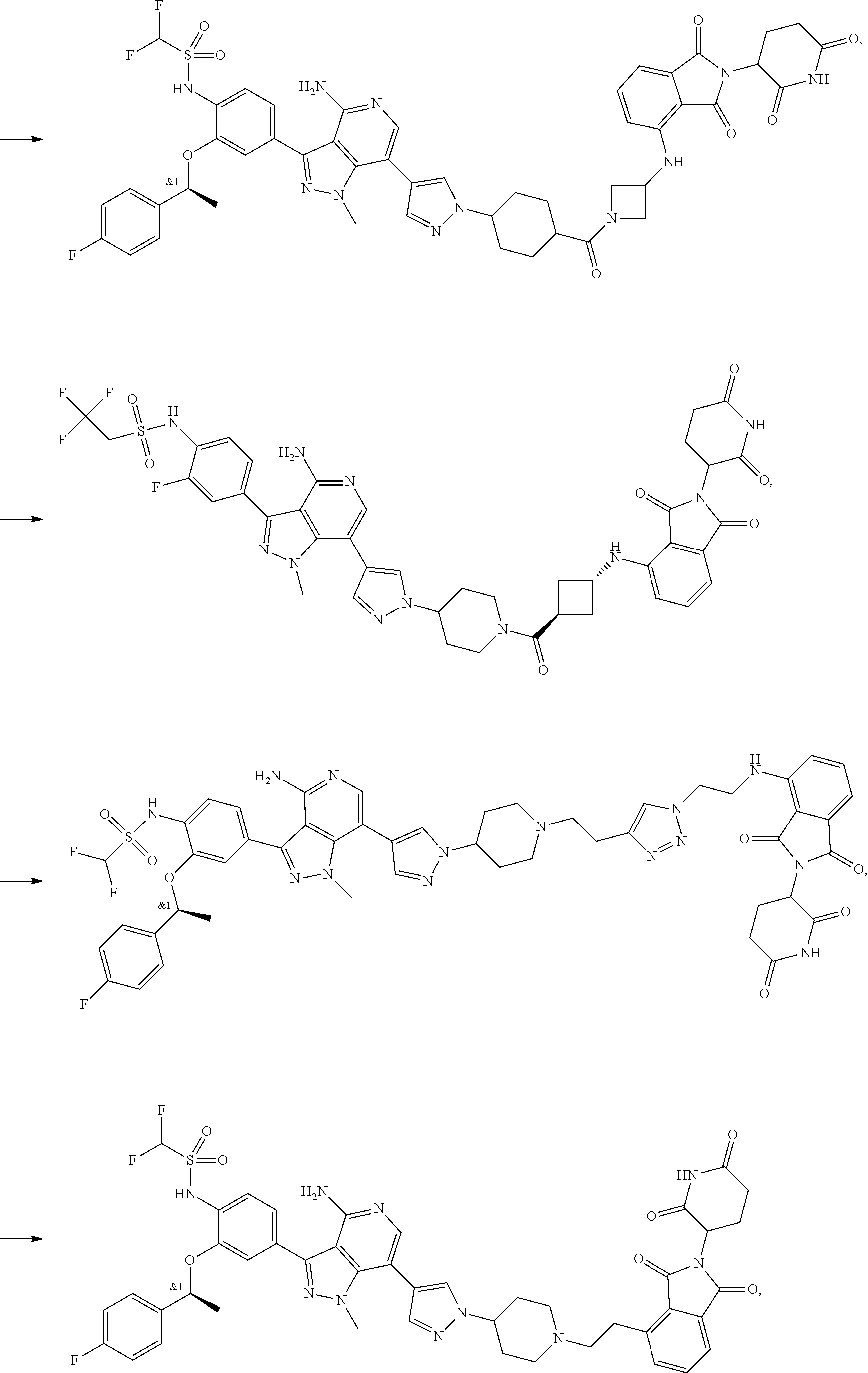 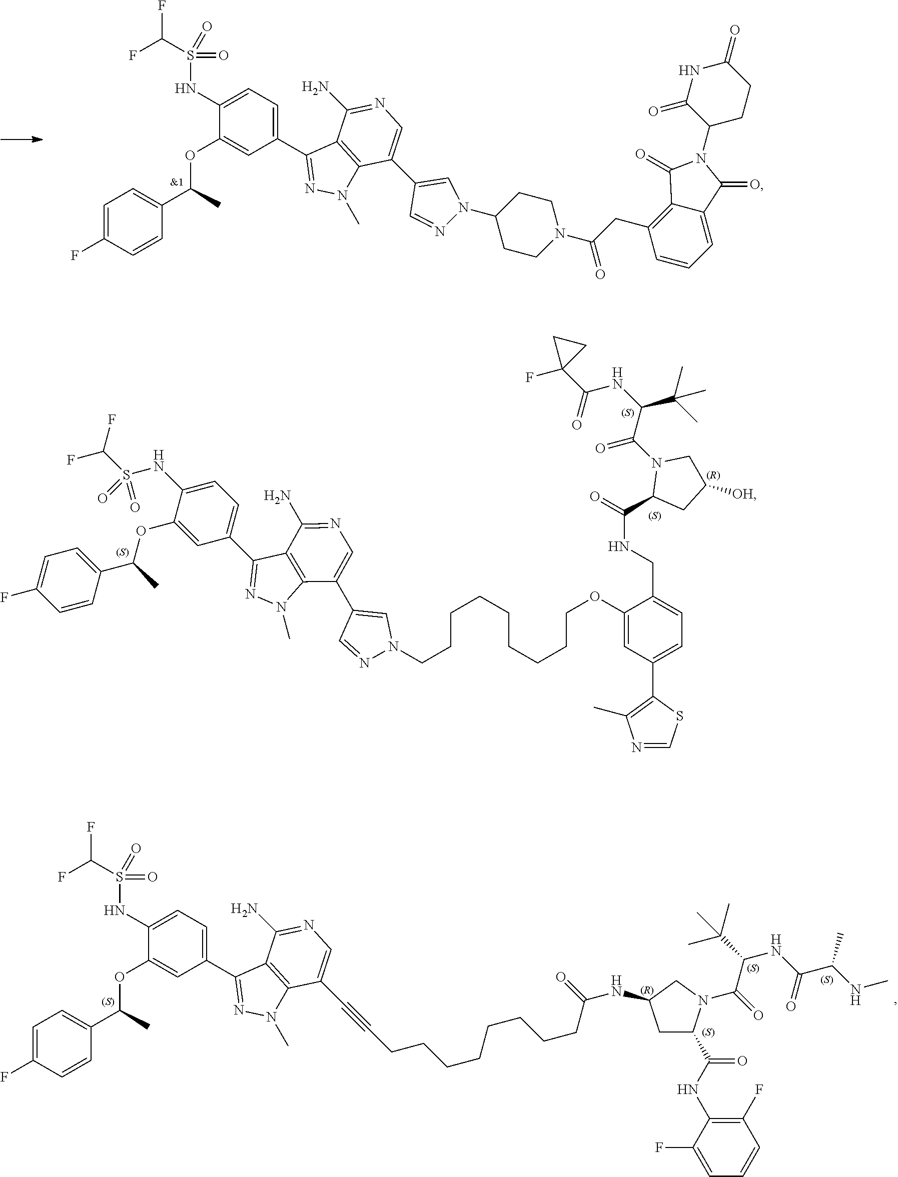 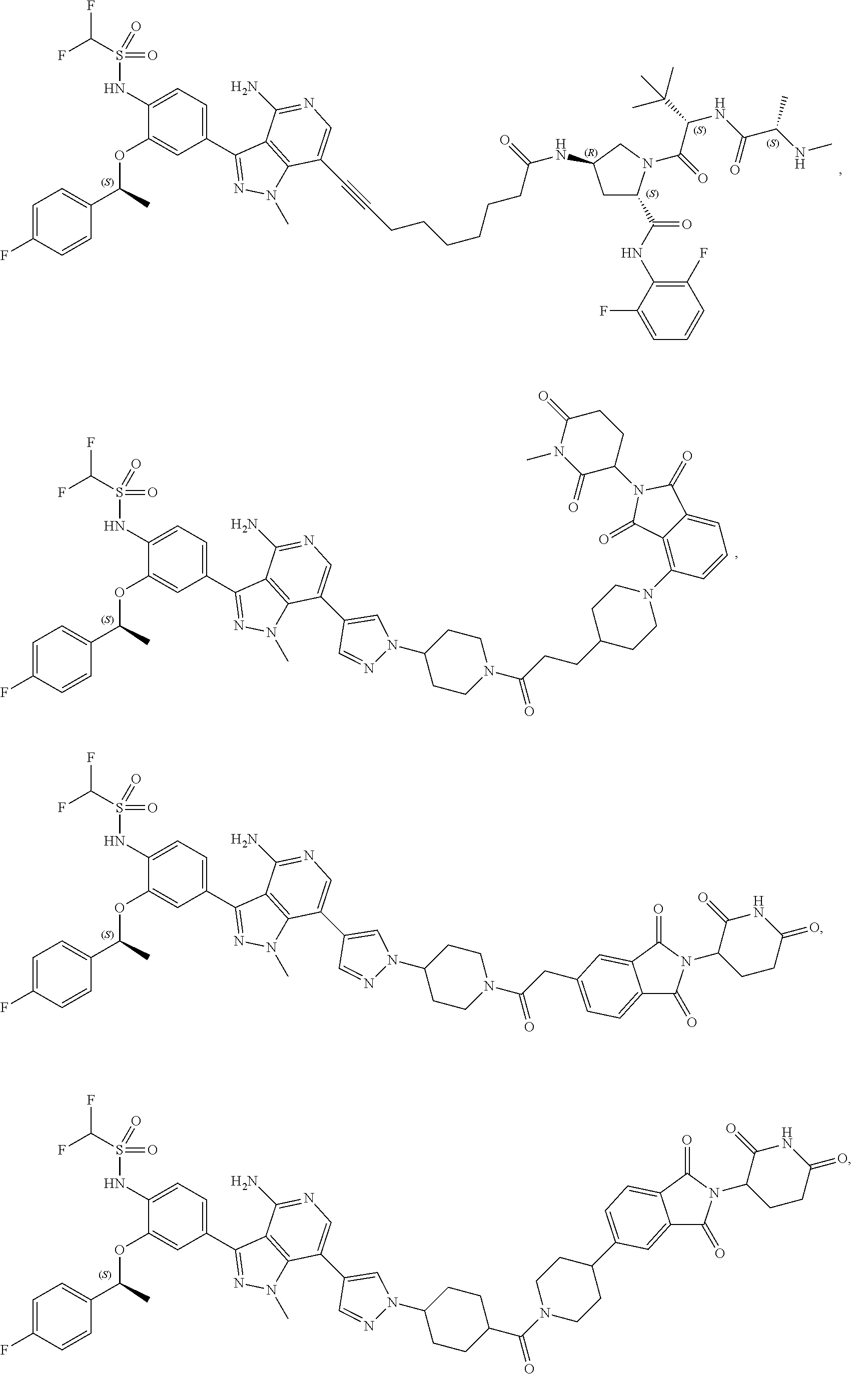 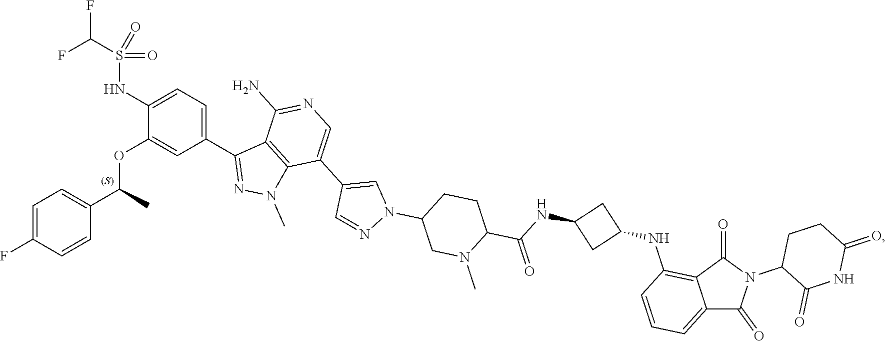
or a pharmaceutically acceptable salt, solvate, tautomer, N-oxide, stereoisomer and/or prodrug thereof.

14. A medicament comprising a compound of any one of claims 1-13, or a pharmaceutically acceptable salt, solvate, tautomer, N-oxide, stereoisomer and/or prodrug thereof.

15. A pharmaceutical composition comprising a compound of any one of claims 1-13, or a pharmaceutically acceptable salt, solvate, tautomer, N-oxide, stereoisomer and/or prodrug thereof, and a pharmaceutically acceptable excipient.

16. A method of treating necroptosis, comprising administering to a subject in need thereof an effective amount of a compound of any one of claims 1-13, or a pharmaceutically acceptable salt, solvate, tautomer, N-oxide, stereoisomer and/or prodrug thereof, the medicament of claim 14, or the pharmaceutical composition of claim 15.

17. The method of claim 16, wherein the subject has a disease selected from the group consisting of diseases of the bones, joints, connective tissue and cartilage, muscular diseases, skin diseases, cardiovascular diseases, circulatory diseases, hematological and vascular diseases, diseases of the lung, diseases of the gastro-intestinal tract, diseases of the liver, diseases of the pancreas, metabolic diseases, diseases of the kidneys, viral and bacterial infections, severe intoxications, degenerative diseases associated with the Acquired Immune Deficiency Syndrome (AIDS), disorders associated with aging, inflammatory diseases, auto-immune diseases, dental disorders, ophthalmic diseases or disorders, diseases of the audition tracts, diseases associated with mitochondria, neuronal loss, ischemic reperfusion injury, diseases of the central nervous system, cancer and metastatic cancer.

18. Use of a compound of any one of claims 1-13, or a pharmaceutically acceptable salt, solvate, tautomer, N-oxide, stereoisomer and/or prodrug thereof, in the manufacture of a medicament for treating necroptosis.

19. A compound of any one of claims 1-13, or a pharmaceutically acceptable salt, solvate, tautomer, N-oxide, stereoisomer and/or prodrug thereof, for use in the treatment of necroptosis.

20. A method of inhibiting and/or degrading mixed lineage kinase domain-like protein (MLKL), comprising contacting a cell with a compound of any one of claims 1-13, or a pharmaceutically acceptable salt, solvate, tautomer, N-oxide, stereoisomer and/or prodrug thereof.

21. A MLKL degrading agent comprising a compound of any one of claims 1-13, or a pharmaceutically acceptable salt, solvate, tautomer, N-oxide, stereoisomer and/or prodrug thereof.
